(19) United States
(12) Patent Application Publication
(10) Pub. No.: US 2023/0192686 A1
(43) Pub. Date: Jun. 22, 2023

(54) 1H-PYRROLO[2,3-B]PYRIDINES AND PREPARATION AND USES THEREOF

(71) Applicant: BioSplice Therapeutics, Inc., San Diego, CA (US)

(72) Inventors: Gopi Kumar Mittapalli, San Diego, CA (US); Sunil Kumar KC, San Diego, CA (US); Chi Ching Mak, San Diego, CA (US); Brian Joseph Hofilena, San Diego, CA (US); Chandramouli Chiruta, San Diego, CA (US); Venkataiah Bollu, San Diego, CA (US); Md Sharif Al Asad, New Haven, CT (US); Brian Walter Eastman, San Diego, CA (US)

(21) Appl. No.: 17/964,423

(22) Filed: Oct. 12, 2022

Related U.S. Application Data
(60) Provisional application No. 63/254,713, filed on Oct. 12, 2021.

(51) Int. Cl. C07D 471/04; C07D 519/00

(52) U.S. Cl. CPC C07D 471/04; C07D 519/00

(57) ABSTRACT

Pyrrolo[2,3-b]pyridine compound for treating various diseases and pathologies are disclosed. More particularly, the present disclosure concerns the use of pyrrolo[2,3-b]pyridine compound or analogs thereof, in the treatment of disorders characterized by overexpression of DYRK1A (e.g., cancer, Down syndrome, Alzheimer's disease, diabetes, viral infections, and osteoarthritis).

### RELATED APPLICATIONS

This application claims the benefit of U.S. Provisional Application No. 63/254,713, filed Oct. 12, 2021, which is incorporated herein by reference in its entirety.

### BACKGROUND

### Technical Field

This disclosure relates to inhibitors of dual-specificity tyrosine phosphorylation-regulated 1A kinase, and compositions comprising the same. More particularly, it concerns the use of a pyrrolo[2,3-b]pyridine compound or salts or analogs thereof, in the treatment of disorders characterized by the abnormal expression and/or activity of DYRK1A (e.g., cancer, Down syndrome, Alzheimer's disease, diabetes, viral infections, and osteoarthritis).

### Background

Dual-specificity tyrosine phosphorylation-regulated kinases (DYRK1A, 1B, 2-4) comprise a family of protein kinases within the CMGC group of the eukaryotic kinome. These protein kinases are involved in multiple cellular functions, including intracellular signaling, mRNA splicing, chromatin transcription, DNA damage repair, cell survival, cell cycle control, differentiation, homocysteine/methionine/folate regulation, body temperature regulation, endocytosis, neuronal development, synaptic plasticity, etc. Abnormal expression and/or activity of some of these kinases, DYRK1A in particular, is seen in many human nervous system diseases, such as cognitive deficits associated with Down syndrome, Alzheimer's disease, and related diseases, tauopathies, dementia, Pick's disease, Parkinson's disease, and other neurodegenerative diseases, Phelan-McDermid syndrome, autism, and CDKL5 deficiency disorder. DYRKs are also involved in diabetes, abnormal folate/methionine metabolism, osteoarthritis, several solid cancers (glioblastoma, breast, and pancreatic cancers) and leukemias (acute lymphoblastic leukemia, acute megakaryoblastic leukemia), viral infections (influenza, HIV-1, HCMV, HCV, CMV, HPV), as well as infections caused by unicellular parasites (Leishmania, Trypanosoma, Plasmodium) (International Journal of Molecular Sciences (2021), 22(11), 6047). DYRK1A has also been identified as a critical stabilizer of EGFR (Cell Death & Disease (2019), 10, 282) which is a crucial factor contributing to the keratinization, cell hyperproliferation, abnormal differentiation and inflammatory infiltration during the progress of psoriasis.

### SUMMARY

The present disclosure provides methods and reagents, involving contacting a cell with an agent, such as an pyrrolo[2,3-b]pyridine compound, in a sufficient amount to antagonize DYRK1A activity, e.g., reduced the proliferation of head and neck squamous cell carcinoma, luminal/HER2 breast cancer (Cell (2016), 164(1-2), 293-309) or pancreatic adenocarcinoma, as well as impaired the self-renewal capacity of glioblastoma and compromised ovarian cancer spheroid cell viability (Molecular Cancer Research (2017), 15(4), 371-381).

The present disclosure also provides methods and reagents, involving contacting a cell with an agent, such as a pyrrolo[2,3-b]pyridine compound, in a sufficient amount to antagonize DYRK1A activity, e.g., i) to normalize prenatal and early postnatal brain development; ii) to improve cognitive function in youth and adulthood; and/or iii) to attenuate Alzheimer's-type neurodegeneration.

Some embodiments disclosed herein include DYRK1A inhibitors containing a pyrrolo[2,3-b]pyridine core. Other embodiments disclosed herein include pharmaceutical compositions and methods of treatment using these compounds.

One embodiment disclosed herein includes a compound having the structure of Formula I:

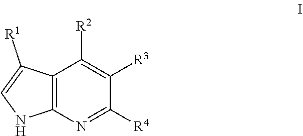

or a pharmaceutically acceptable salt thereof,

wherein:

R¹ is selected from the group consisting of 6-membered heteroaryl optionally substituted with 1-4 R⁵ and 7-14-membered heteroaryl optionally substituted with 1-10 R⁶;

wherein a carbon atom on an aromatic ring of the heteroaryl R¹ group forms the bond with

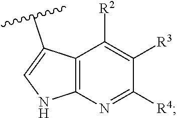

R² is selected from the group consisting of H, —OR⁷, and 5-membered heteroaryl optionally substituted with 1-3 R⁸, and —NHR⁹;

R³ is selected from the group consisting of H, halide, CN, —CO₂H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚheterocyclyl optionally substituted with 1-10 R¹¹, -(5-9 membered heteroaryl) optionally substituted with 1-10 R¹²; phenyl optionally substituted with 1-5 R¹³, —NHheteroaryl optionally substituted with 1-10 R¹⁴; —C(═O)NHR¹⁵, —NH(C═O)R¹⁶, and —C(═O)R¹⁷;

R⁴ is selected from the group consisting of H, halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, and —C(═O)R²⁵;

with the proviso that at least two of R², R³, and R⁴ are H;

each R⁵ is independently selected from the group consisting of —OR²⁶, -heterocyclyl optionally substituted with 1-10 R²⁷, and —NHheterocyclyl optionally substituted with 1-10 R²⁸;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is —CH₂(6-membered heteroaryl) optionally substituted with 1-4 R³⁹;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R¹³ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R¹⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

R¹⁵ is selected from the group consisting of -heterocyclyl optionally substituted with 1-10 R³⁷ and -heteroaryl optionally substituted with 1-10 R³⁹;

R¹⁶ is selected from the group consisting of -heterocyclyl optionally substituted with 1-10 R³⁷ and -heteroaryl optionally substituted with 1-10 R³⁹;

R¹⁷ is selected from the group consisting of -heterocyclyl optionally substituted with 1-10 R³⁷ and -heteroaryl optionally substituted with 1-10 R³⁹;

R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;

each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R²³ is selected from the group consisting of -heterocyclyl optionally substituted with 1-10 R³⁷ and -heteroaryl optionally substituted with 1-10 R³⁹;

R²⁴ is selected from the group consisting of -heterocyclyl optionally substituted with 1-10 R³⁷ and -heteroaryl optionally substituted with 1-10 R³⁹;

R²⁵ is selected from the group consisting of -heterocyclyl optionally substituted with 1-10 R³⁷ and -heteroaryl optionally substituted with 1-10 R³⁹;

each R²⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each m is independently 1 to 6; and

each p is independently 0 or 1.

Some embodiments include stereoisomers and pharmaceutically acceptable salts of a compound of Formula I. Some embodiments include pharmaceutically acceptable salts of a compound of Formula I.

Some embodiments include pro-drugs of a compound of Formula I.

Some embodiments of the present disclosure include pharmaceutical compositions comprising a compound of Formula I and a pharmaceutically acceptable carrier, diluent, or excipient.

Other embodiments disclosed herein include methods of inhibiting DYRK1A by administering to a patient affected by a disorder or disease in which DYRK1A overexpression is implicated, such as Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor and Stroke.

Inhibitors of DYRK1A can also be used to treat tauopathies. Tauopathies are neurodegenerative disorders characterized by the deposition of abnormal tau protein in the brain. The spectrum of tau pathologies expands beyond the traditionally discussed disease forms like Pick's disease, progressive supranuclear palsy, corticobasal degeneration, and argyrophilic grain disease. Emerging entities and pathologies include globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia (Handbook of Clinical Neurology (2018), 145, 355-368 and Aging Cell (2019), 18(5), e13000).

Inhibitors of DYRK1A can also be used to treat disorders associated with abnormal folate/methionine metabolism.

Non-limiting examples of diseases which can be treated with the compounds and compositions provided herein include a variety of cancers, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

Some embodiments of the present disclosure include methods to prepare compounds of Formula I.

It is to be understood that both the foregoing general description and the following detailed description are exemplary and explanatory only and are not restrictive of the disclosure, as claimed.

### DETAILED DESCRIPTION

Provided herein are compositions and methods for inhibiting DYRK1A.

Some embodiments provided herein relate to a method for treating a disease including, but not limited to, neurological diseases or disorders, cancers, cognitive deficits, knee osteoarthritis, tendinopathy, viral infections, unicellular parasite infections, and motor deficits.

In some embodiments, non-limiting examples of a neurological disease or disorder which can be treated with the compounds and compositions provided herein include, but are not limited to, Alzheimer's disease, amyotrophic lateral sclerosis, Down Syndrome, frontotemporal dementia with Parkinsonism-17 (FTDP-17), Lewy body dementia, Parkinson's disease, Pick's disease tauopathies, and additional diseases with pronounced neurodegeneration such as autism, dementia, epilepsy, Huntington's disease, multiple sclerosis; diseases and disorders associated with acquired brain injury such as Chronic Traumatic Encephalopathy, Traumatic Brain Injury, Tumor, and Stroke.

In some embodiments, non-limiting examples of cancers which can be treated with the compounds and compositions provided herein include solid cancers (e.g., glioblastoma, ovarian, breast, and pancreatic cancers) and leukemias (e.g., acute lymphoblastic leukemia, acute megakaryoblastic leukemia, and chronic myeloid leukemia).

In some embodiments, pharmaceutical compositions are provided that are effective for treatment of a disease of an animal, e.g., a mammal, caused by DYRK1A overexpression. The composition includes a pharmaceutically acceptable carrier and a compound as described herein.

Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-O— group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “haloalkoxy” means a haloalkyl-O— group in which the haloalkyl group is as described herein. Exemplary haloalkoxy groups include fluoromethoxy, difluoromethoxy, trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylalkylene” means an aryl-alkylene-group in which the aryl and alkylene moieties are as previously described. In some embodiments, arylalkylene groups contain a C₁₋₄alkylene moiety. Exemplary arylalkylene groups include benzyl and 2-phenethyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine , isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched, alkyl, alkenyl or alkynyl substituted with one or more chloro, bromo, fluoro, and/or iodo atom(s). In some embodiments, a haloalkyl is a fluoroalkyls, wherein one or more of the hydrogen atoms have been substituted by fluoro. In some embodiments, haloalkyls are of 1 to 3 carbons in length (e.g., 1 to 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings such as bicyclic and spirocyclic heterocyclyls. Heterocyclyls may be substituted or unsubstituted with one or more substituents. In some embodiments, heterocycles have 3-11 members. In six membered monocyclic heterocycles, the heteroatom(s) are selected from one to three of O, N and S, and wherein when the heterocycle is five membered, it can have one or two heteroatoms selected from O, N, and S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 3-7 members. In six membered monocyclic heterocycles, the heteroatom(s) are selected from one to three of O, N and S, and wherein when the heterocycle is five membered, it can have one or two heteroatoms selected from O, N, and S. Examples of monocyclic heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents. In some embodiments, bicyclic heterocycles have 4-11 members with the heteroatom(s) being selected from one to five of O, N and S. Examples of bicyclic heterocyclyls include 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, and the like.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N and S. Examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with 1-3 R″′; 5-10 membered heteroaryl having from 1-4 heteroatoms independently selected from N, O, and S and optionally substituted with 1-3 R″′; C₃₋₇ carbocyclyl optionally substituted with 1-3 R″′; and 3-8 membered heterocyclyl having from 1-4 heteroatoms independently selected from N, O, and S and optionally substituted with 1-3 R′″; wherein each R″′ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formula I wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), isotopes of carbon, such as ¹¹C, ¹³C and ¹⁴C, isotopes of chlorine, such as ³⁶Cl, isotopes of fluorine, such as ¹⁸F, isotopes of iodine, such as ¹²³I and ¹²⁵I, isotopes of nitrogen, such as ¹³N and ¹⁵N, isotopes of oxygen, such as ¹⁵O, ¹⁷O and ¹⁸O, isotopes of phosphorus, such as ³²P, and isotopes of sulfur, such as ³⁵S.

The term “administration” or “administering” refers to a method of providing a dosage of a compound or pharmaceutical composition to a vertebrate or invertebrate, including a mammal, a bird, a fish, or an amphibian, where method of administration is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” and “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formula I in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age, and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing, or preventing the further development of a disorder, and/or reducing the severity of symptoms that will or are expected to develop.

“Drug-eluting” and/or controlled release as used herein refers to any and all mechanisms, e.g., diffusion, migration, permeation, and/or desorption by which the drug(s) incorporated in the drug-eluting material pass therefrom over time into the surrounding body tissue.

“Drug-eluting material” and/or controlled release material as used herein refers to any natural, synthetic, or semi-synthetic material capable of acquiring and retaining a desired shape or configuration and into which one or more drugs can be incorporated and from which incorporated drug(s) are capable of eluting over time.

“Elutable drug” as used herein refers to any drug or combination of drugs having the ability to pass over time from the drug-eluting material in which it is incorporated into the surrounding areas of the body.

Compounds

The compounds and compositions described herein can be used to inhibit DYRK1A for treating a disorder or disease in which DYRK1A overexpression is implicated, such as in neurological diseases or disorders, cancers, cognitive deficits, knee osteoarthritis, tendinopathy, viral infections, unicellular parasite infections, and motor deficits.

Some embodiments of the present disclosure include compounds of Formula I:

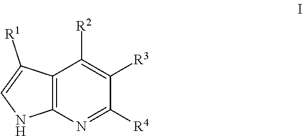

or salts, pharmaceutically acceptable salts, or prodrugs thereof,

wherein:

R¹ is pyridinyl substituted with 1-4 R⁵;

R² is selected from the group consisting of H, —OR⁷, 5-membered heteroaryl optionally substituted with 1-3 R⁸, and —NHR⁹.

R³ is selected from the group consisting of H, CN, —CO₂H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚheterocyclyl optionally substituted with 1-10 R¹¹, -(5-9 membered heteroaryl) optionally substituted with 1-10 R¹²; phenyl optionally substituted with 1-5 R¹³, —NHheteroaryl optionally substituted with 1-10 R¹⁴; —C(═O)NHR¹⁵, —NH(C═O)R¹⁶, and —C(═O)R¹⁷;

R⁴ is selected from the group consisting of H, halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, and —C(═O)R²⁵;

with the proviso that at least two of R², R³, and R⁴ are H;

each R⁵ is independently selected from the group consisting of —OR²⁶, -heterocyclyl optionally substituted with 1-10 R²⁷, and —NHheterocyclyl optionally substituted with 1-10 R²⁸;

R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is —CH₂(6-membered heteroaryl) optionally substituted with 1-4 R³⁹;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R¹³ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R¹⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

R¹⁵ is selected from the group consisting of -heterocyclyl optionally substituted with 1-10 R³⁷ and -heteroaryl optionally substituted with 1-10 R³⁹;

R¹⁶ is selected from the group consisting of -heterocyclyl optionally substituted with 1-10 R³⁷ and -heteroaryl optionally substituted with 1-10 R³⁹;

R¹⁷ is selected from the group consisting of -heterocyclyl optionally substituted with 1-10 R³⁷ and -heteroaryl optionally substituted with 1-10 R³⁹;

R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;

each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R²³ is selected from the group consisting of -heterocyclyl optionally substituted with 1-10 R³⁷ and -heteroaryl optionally substituted with 1-10 R³⁹;

R²⁴ is selected from the group consisting of -heterocyclyl optionally substituted with 1-10 R³⁷ and -heteroaryl optionally substituted with 1-10 R³⁹;

R²⁵ is selected from the group consisting of -heterocyclyl optionally substituted with 1-10 R³⁷ and -heteroaryl optionally substituted with 1-10 R³⁹;

each R²⁶ is unsubstituted —(C₁₋₉ haloalkyl);

each R²⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each p is independently 0 or 1; and

with the proviso that Formula I is not a structure selected from the group consisting of:

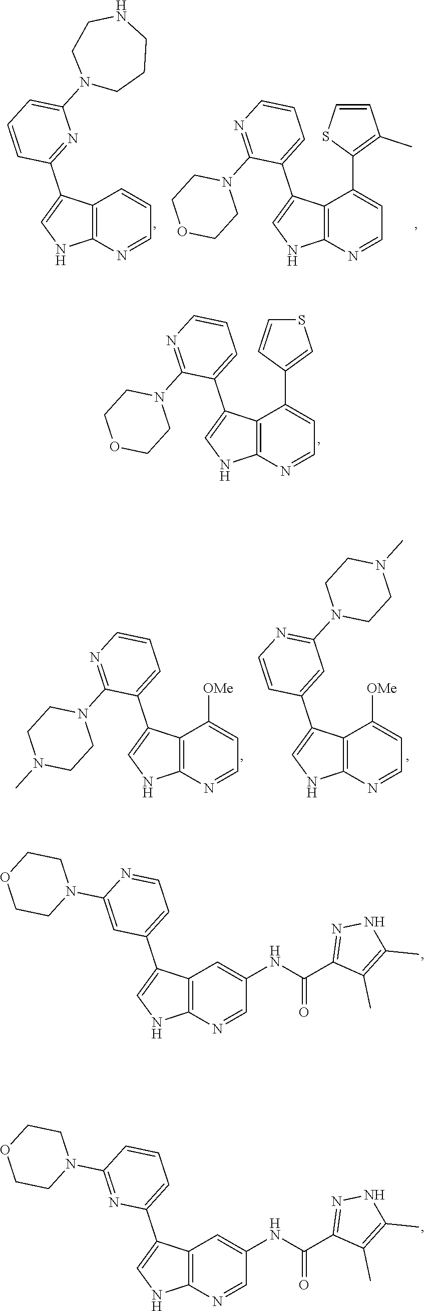

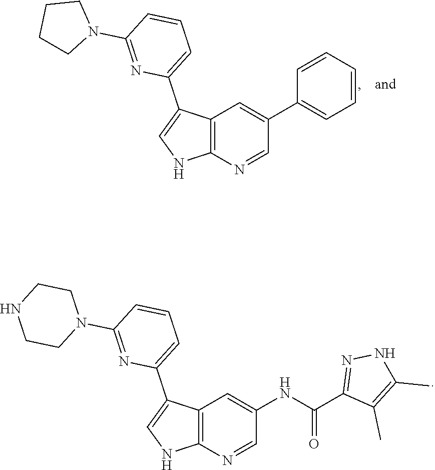 [SMILES: c1ccc(-c2cnc3[nH]cc(-c4cccc(N5CCCC5)n4)c3c2)cc1 ; Cc1[nH]nc(C(=O)Nc2cnc3[nH]cc(-c4cccc(N5CCNCC5)n4)c3c2)c1C]

Some embodiments of the present disclosure include compounds of Formula Ia:

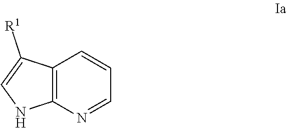

or salts, pharmaceutically acceptable salts, or prodrugs thereof,

wherein:

R¹ is selected from the heteroaryl group consisting of:

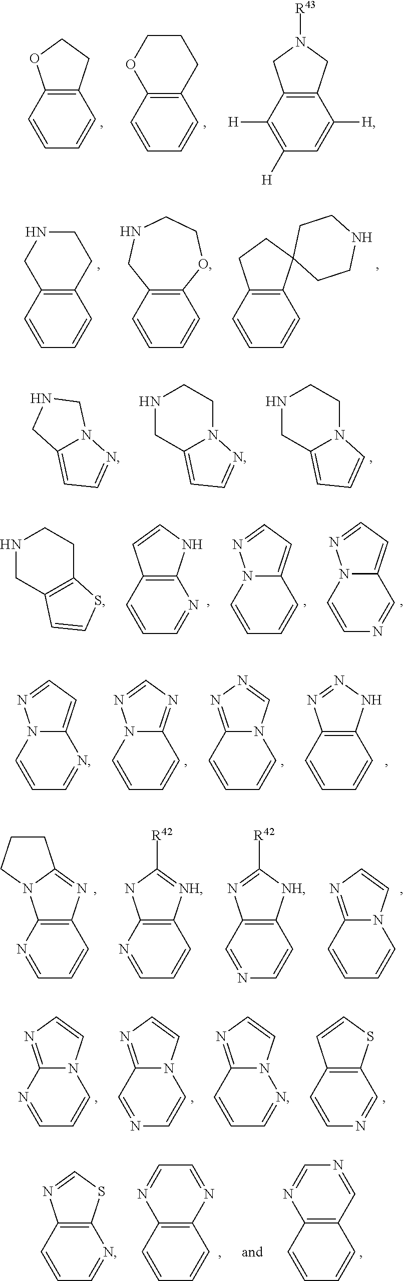

and optionally substituted with 1-10 R⁶; and

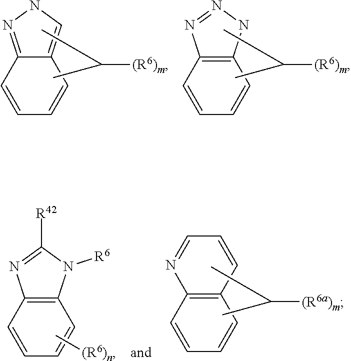

wherein a carbon atom on an aromatic ring of the heteroaryl form the bond with

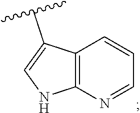 [SMILES: *c1c[nH]c2ncccc12]

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

each R^{6a} is independently selected from the group consisting of —OR^{29a}, —CH₂OH, -heterocyclyl optionally substituted with 1-10 R³⁰, —CH₂N(R³⁴)₂, —C(═O)N(R³⁵)₂, and —C(═O)R³⁶;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R^{29a} is -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each n is independently 0 to 3;

each m is independently 1 to 6;

each p is independently 0 or 1; and

with the proviso that Formula Ia is not a structure selected from the group consisting of:

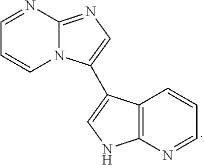 [SMILES: c1cnc2[nH]cc(-c3cnc4ncccn34)c2c1]

Some embodiments of the present disclosure include compounds of Formula Ib:

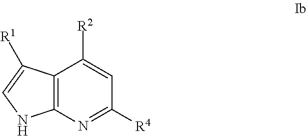

or salts, pharmaceutically acceptable salts, or prodrugs thereof,

wherein:

R¹ is 7-14-membered heteroaryl optionally substituted with 1-10 R⁶;

with the proviso that when the heteroaryl is selected from the group consisting of

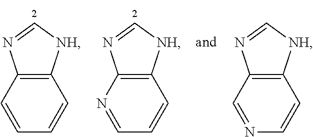

that the 2-position is substituted with R⁴²;

wherein a carbon atom on an aromatic ring of the heteroaryl form the bond with

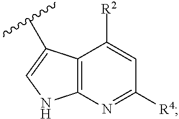

R² is selected from the group consisting of H, —OR⁷, 5-membered heteroaryl optionally substituted with 1-3 R⁸, and —NHR⁹;

R⁴ is selected from the group consisting of H, halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, and —C(═O)R²⁵;

with the proviso that one of R² and R⁴ is H and the other of R² and R⁴ is not H;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is —CH₂(6-membered heteroaryl) optionally substituted with 1-4 R³⁹;

R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;

each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R²³ is selected from the group consisting of -heterocyclyl optionally substituted with 1-10 R³⁷ and -heteroaryl optionally substituted with 1-10 R³⁹;

R²⁴ is selected from the group consisting of -heterocyclyl optionally substituted with 1-10 R³⁷ and -heteroaryl optionally substituted with 1-10 R³⁹;

R²⁵ is selected from the group consisting of -heterocyclyl optionally substituted with 1-10 R³⁷ and -heteroaryl optionally substituted with 1-10 R³⁹;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each m is independently 1 to 6;

each p is independently 0 or 1; and

with the proviso that Formula Ib is not a structure selected from the group consisting of:

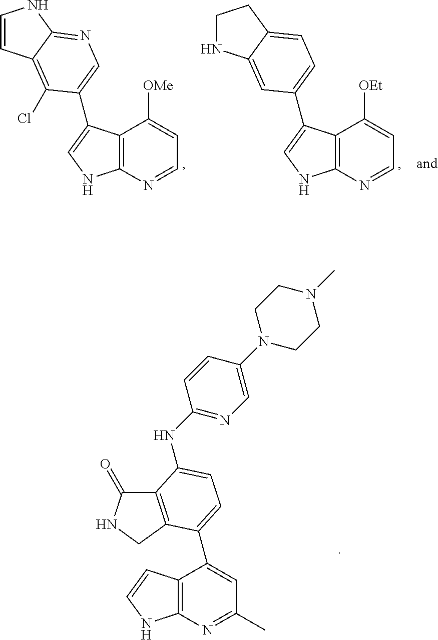 [SMILES: COc1ccnc2[nH]cc(-c3cnc4[nH]ccc4c3Cl)c12 ; CCOc1ccnc2[nH]cc(-c3ccc4c(c3)NCC4)c12 ; Cc1cc(-c2ccc(Nc3ccc(N4CCN(C)CC4)cn3)c3c2CNC3=O)c2cc[nH]c2n1]

Some embodiments of the present disclosure include compounds of Formula Ic:

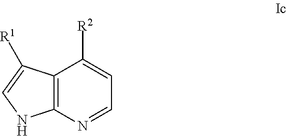

or salts, pharmaceutically acceptable salts, or prodrugs thereof,

wherein:

R¹ is 7-14-membered heteroaryl optionally substituted with 1-10 R⁶;

wherein a carbon atom on an aromatic ring of the heteroaryl form the bond with

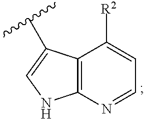

R² is selected from the group consisting of H, —OR⁷, 5-membered heteroaryl optionally substituted with 1-3 R⁸, and —NHR⁹,

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is —CH₂(6-membered heteroaryl) optionally substituted with 1-4 R³⁹;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉haloalkyl), and -heterocyclyl optionally substituted with 1-10 R³⁷;

each m is independently 1 to 6;

each p is independently 0 or 1; and

with the proviso that Formula Ic is not a structure selected from the group consisting of:

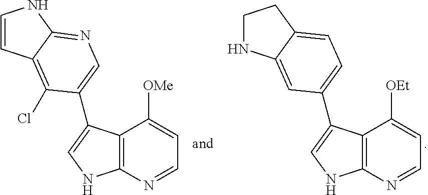 [SMILES: COc1ccnc2[nH]cc(-c3cnc4[nH]ccc4c3Cl)c12 ; CCOc1ccnc2[nH]cc(-c3ccc4c(c3)NCC4)c12]

Some embodiments of the present disclosure include compounds of Formula Ic:

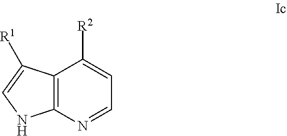

or salts, pharmaceutically acceptable salts, or prodrugs thereof,

wherein:

R¹ is selected from the heteroaryl group consisting of:

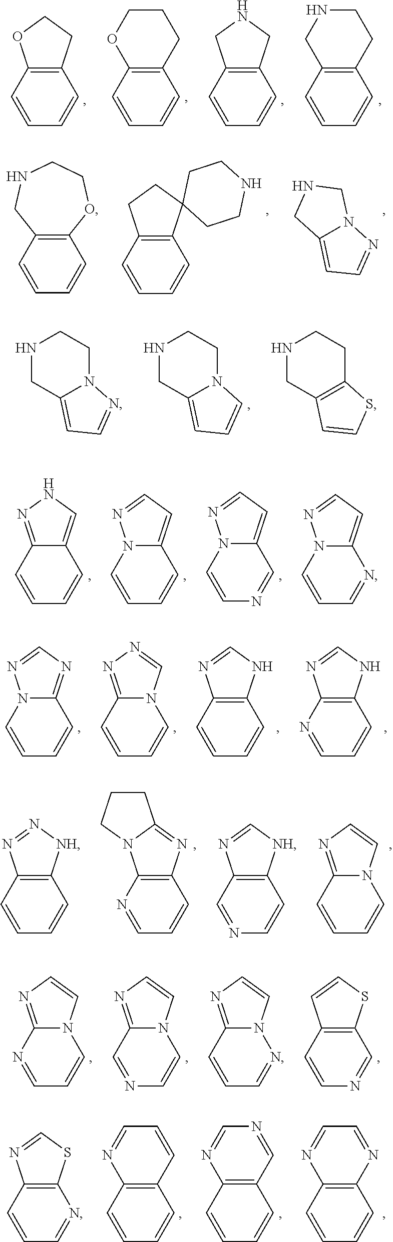

and optionally substituted with 1-10 R⁶; and

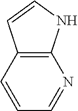

is optionally substituted with 1-4 R^{6a};

wherein a carbon atom on an aromatic ring of the heteroaryl form the bond with

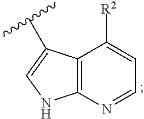

R² is selected from the group consisting of H, —OR⁷, 5-membered heteroaryl optionally substituted with 1-3 R⁸, and —NHR⁹,

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

each R^{6a} is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R⁹ is —CH₂(6-membered heteroaryl) optionally substituted with 1-4 R³⁹;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each m is independently 1 to 6; and

each p is independently 0 or 1.

Some embodiments of the present disclosure include compounds of Formula Id:

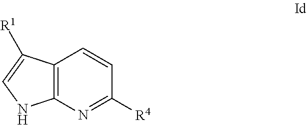

or salts, pharmaceutically acceptable salts, or prodrugs thereof,

wherein:

R¹ is 7-14-membered heteroaryl optionally substituted with 1-10 R⁶;

with the proviso that when the heteroaryl is selected from the group consisting of

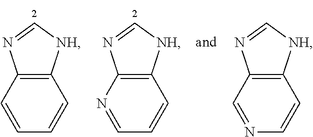

that the 2-position is substituted with R⁴²;

wherein a carbon atom on an aromatic ring of the heteroaryl form the bond with

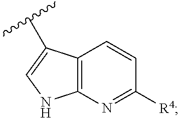

R⁴ is selected from the group consisting of halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, and —C(═O)R²⁵;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;

each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R²³ is selected from the group consisting of -heterocyclyl optionally substituted with 1-10 R³⁷ and -heteroaryl optionally substituted with 1-10 R³⁹;

R²⁴ is selected from the group consisting of -heterocyclyl optionally substituted with 1-10 R³⁷ and -heteroaryl optionally substituted with 1-10 R³⁹;

R²⁵ is selected from the group consisting of -heterocyclyl optionally substituted with 1-10 R³⁷ and -heteroaryl optionally substituted with 1-10 R³⁹;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each m is independently 1 to 6;

each p is independently 0 or 1; and

with the proviso that Formula Id is not a structure selected from the group consisting of:

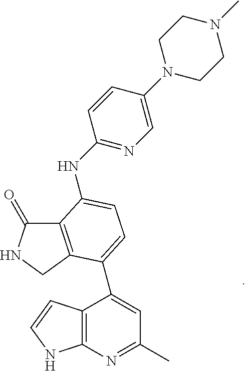 [SMILES: Cc1cc(-c2ccc(Nc3ccc(N4CCN(C)CC4)cn3)c3c2CNC3=O)c2cc[nH]c2n1]

Some embodiments of the present disclosure include compounds of Formula Id:

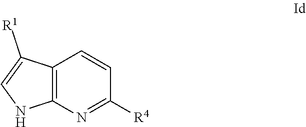

or salts, pharmaceutically acceptable salts, or prodrugs thereof,

wherein:

R¹ is selected from the heteroaryl group consisting of:

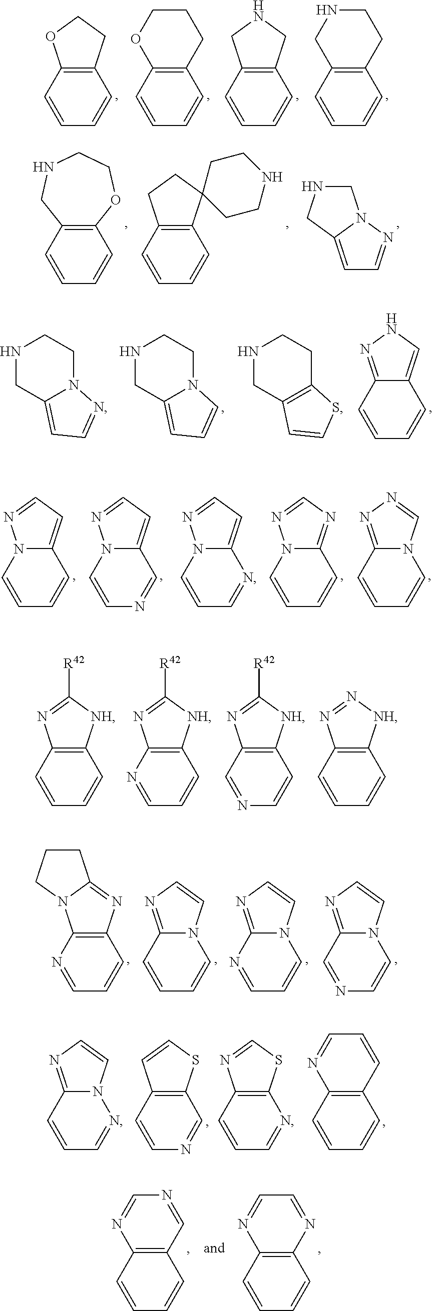

and optionally substituted with 1-10 R⁶;

wherein a carbon atom on an aromatic ring of the heteroaryl form the bond with

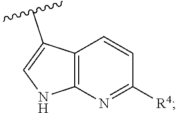

R⁴ is selected from the group consisting of halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, —C(═O)R²⁵;

each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, —C(═O)R³⁶;

alternatively, two R⁶ attached to the same carbon atom to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;

each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

R²³ is selected from the group consisting of -heterocyclyl optionally substituted with 1-10 R³⁷ and -heteroaryl optionally substituted with 1-10 R³⁹;

R²⁴ is selected from the group consisting of -heterocyclyl optionally substituted with 1-10 R³⁷ and -heteroaryl optionally substituted with 1-10 R³⁹;

R²⁵ is selected from the group consisting of -heterocyclyl optionally substituted with 1-10 R³⁷ and -heteroaryl optionally substituted with 1-10 R³⁹;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each m is independently 1 to 6;

each p is independently 0 or 1; and

with the proviso that Formula Id is not a structure selected from the group consisting of:

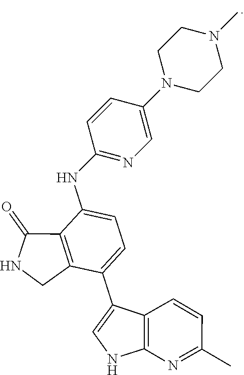 [SMILES: Cc1ccc2c(-c3ccc(Nc4ccc(N5CCN(C)CC5)cn4)c4c3CNC4=O)c[nH]c2n1]

Some embodiments of the present disclosure include compounds of Formula Ie:

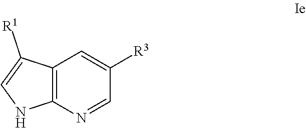

or salts, pharmaceutically acceptable salts, or prodrugs thereof,

wherein:

R¹ is selected from the heteroaryl group consisting of:

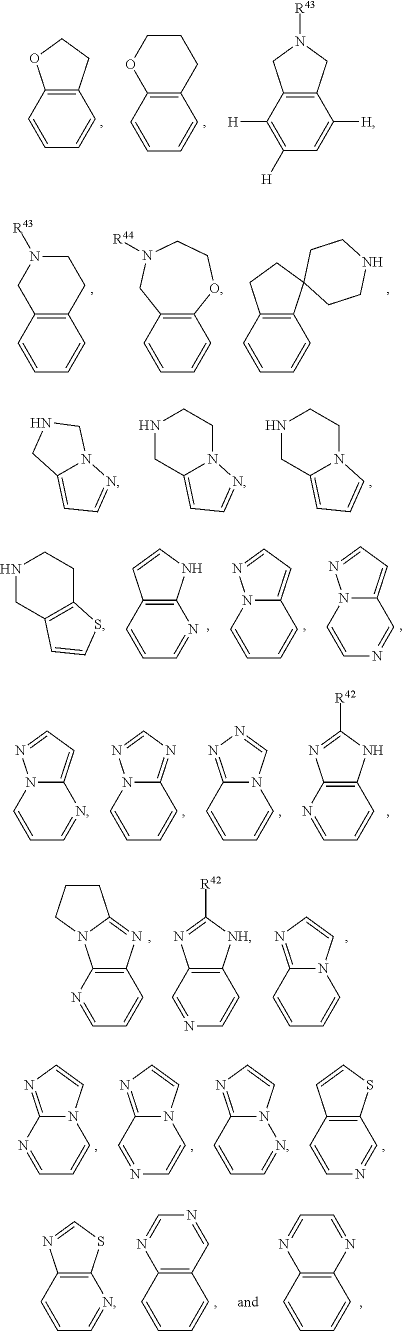

and optionally substituted with 1-10 R⁶; and

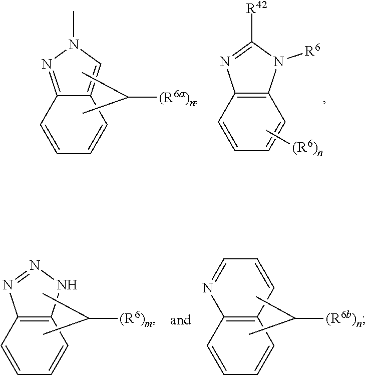

with the proviso that when R¹ is

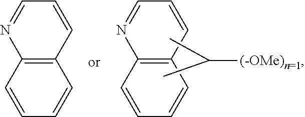

R³ is not halide or methyl;

wherein a carbon atom on an aromatic ring of the heteroaryl form the bond with

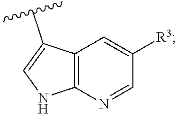

R³ is selected from the group consisting of halide, CN, —CO₂H, —OR¹⁰, unsubstituted —(C₁₋₉ alkyl), unsubstituted —(C₂₋₉ alkenyl), unsubstituted —(C₂₋₉ alkenyl), unsubstituted —(C₁₋₉haloalkyl), —(CH₂)ₚheterocyclyl optionally substituted with 1-10 R¹¹, -(5-9 membered heteroaryl) optionally substituted with 1-10 R¹²; phenyl optionally substituted with 1-5 R¹³, —NHheteroaryl optionally substituted with 1-10 R¹⁴; —C(═O)NHR¹⁵, —NH(C═O)R¹⁶, and —C(═O)R¹⁷;

each R⁶ is independently selected from the group consisting of halide, unsubstituted —(C₁₋₉ alkyl), unsubstituted —(C₂₋₉ alkenyl), unsubstituted —(C₂₋₉alkynyl), unsubstituted —(C₁₋₉haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

alternatively, two R⁶ attached to the same carbon atom are taken together to form a carbonyl group;

alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;

each R^{6a} is independently selected from the group consisting of halide, unsubstituted —(C₂₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;

each R^{6b} is independently selected from the group consisting of —OR²⁹, —CH₂OH, -heterocyclyl optionally substituted with 1-10 R³⁰, —CH₂N(R³⁴)₂, —C(═O)N(R³⁵)₂, and —C(═O)R³⁶;

R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R¹³ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R¹⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

R¹⁵ is selected from the group consisting of -heterocyclyl optionally substituted with 1-10 R³⁷ and -heteroaryl optionally substituted with 1-10 R³⁹;

R¹⁶ is selected from the group consisting of -heterocyclyl optionally substituted with 1-10 R³⁷ and -heteroaryl optionally substituted with 1-10 R³⁹;

R¹⁷ is selected from the group consisting of -heterocyclyl optionally substituted with 1-10 R³⁷ and -heteroaryl optionally substituted with 1-10 R³⁹;

each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;

each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;

each R³⁶ is -heterocyclyl optionally substituted with 1-10 R⁴¹;

each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;

each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;

each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;

alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;

each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R⁴³ is H;

each R⁴⁴ is independently selected from the group consisting of H, unsubstituted —(C₂₋₉ alkyl), unsubstituted —(C₂₋₉ alkenyl), unsubstituted —(C₂₋₉ alkynyl), and unsubstituted —(C₁₋₉ haloalkyl);

each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;

each n is independently 0 to 6;

each m is independently 1 to 6;

each p is independently 0 or 1; and

with the proviso that Formula Ie is not a structure selected from the group consisting of:

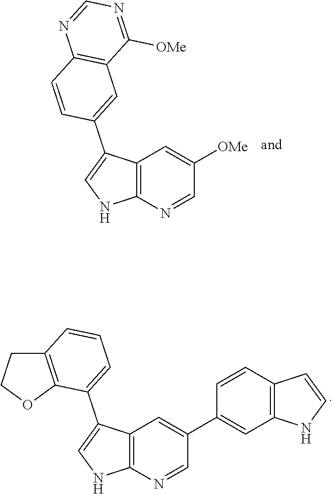 [SMILES: COc1cnc2[nH]cc(-c3ccc4ncnc(OC)c4c3)c2c1 ; c1cc2c(c(-c3c[nH]c4ncc(-c5ccc6cc[nH]c6c5)cc34)c1)OCC2]

Some embodiments of the present disclosure include compounds of Formula I:

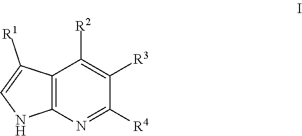

or salts, pharmaceutically acceptable salts, or prodrugs thereof.

Some embodiments of the present disclosure include compounds of Formula Ia:

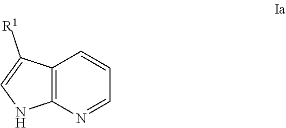

or salts, pharmaceutically acceptable salts, or prodrugs thereof.

Some embodiments of the present disclosure include compounds of Formula Ib:

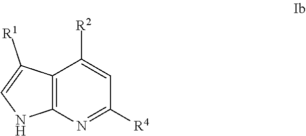

or salts, pharmaceutically acceptable salts, or prodrugs thereof.

Some embodiments of the present disclosure include compounds of Formula Ic:

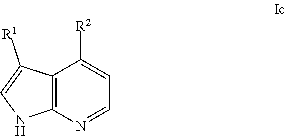

or salts, pharmaceutically acceptable salts, or prodrugs thereof.

Some embodiments of the present disclosure include compounds of Formula Id:

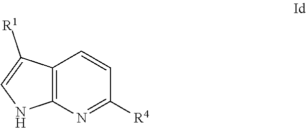

or salts, pharmaceutically acceptable salts, or prodrugs thereof.

Some embodiments of the present disclosure include compounds of Formula Ie:

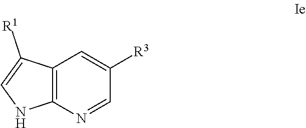

or salts, pharmaceutically acceptable salts, or prodrugs thereof.

In some embodiments of Formula I, R¹ is selected from the group consisting of 6-membered heteroaryl optionally substituted with 1-4 (e.g., 1-3, 1-2, 1) R⁵ and 7-14-membered heteroaryl optionally substituted with 1-10 (e.g., 1-9, 1-8, 1-7, 1-6, 1-5, 1-4, 1-3, 1-2, 1) R⁶.

In some embodiments of Formula I, R¹ is pyridinyl substituted with 1-4 (e.g., 1-3, 1-2, 1) R⁵.

In some embodiments of Formula I, R¹ is 4-pyridine substituted with 1 R⁵.

In some embodiments of Formula Ia, R¹ is selected from the heteroaryl group consisting of:

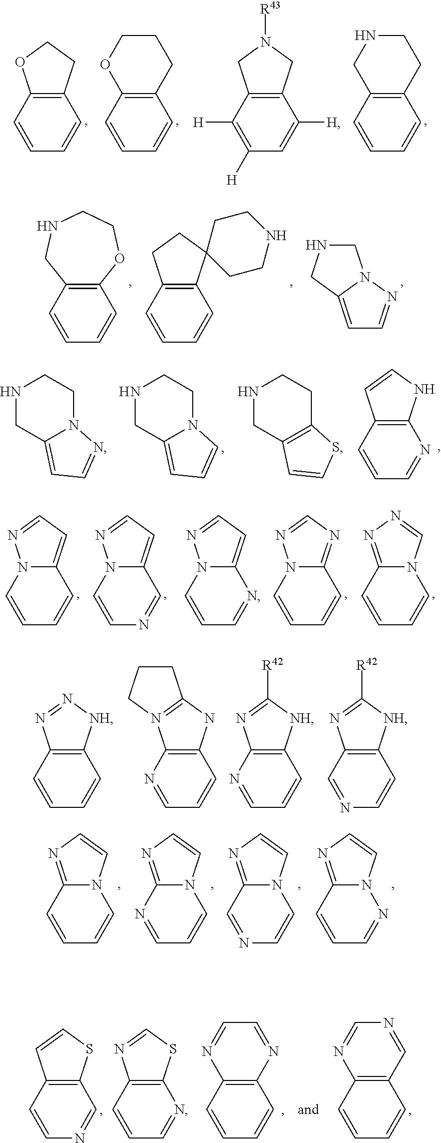

and optionally substituted with 1-10 (e.g., 1-9, 1-8, 1-7, 1-6, 1-5, 1-4, 1-3, 1-2, 1) R⁶; and

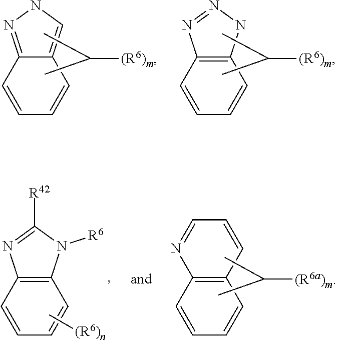

In some embodiments of Formulas Ib, Ic, and Id, R¹ is 7-14-membered heteroaryl optionally substituted with 1-10 (e.g., 1-9, 1-8, 1-7, 1-6, 1-5, 1-4, 1-3, 1-2, 1) R⁶.

In some embodiments of Formulas Ib and Id, there is the proviso that when the heteroaryl is selected from the group consisting of

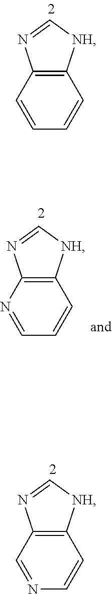 [SMILES: c1ccc2[nH]cnc2c1 ; c1cnc2nc[nH]c2c1 ; c1cc2[nH]cnc2cn1]

that the 2-position is substituted with R⁴².

In some embodiments of Formula Ic, R¹ is selected from the heteroaryl group consisting of:

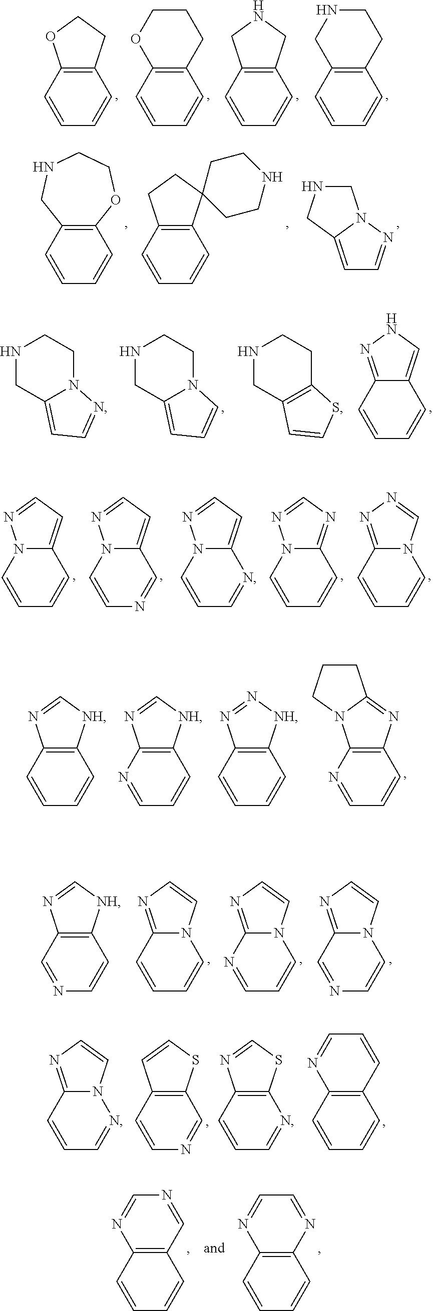

and optionally substituted with 1-10 (e.g., 1-9, 1-8, 1-7, 1-6, 1-5, 1-4, 1-3, 1-2, 1) R⁶; and

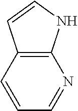

is optionally substituted with 1-4 (e.g., 1-3, 1-2, 1) R^{6a}.

In some embodiments of Formula Id, R¹ is selected from the heteroaryl group consisting of:

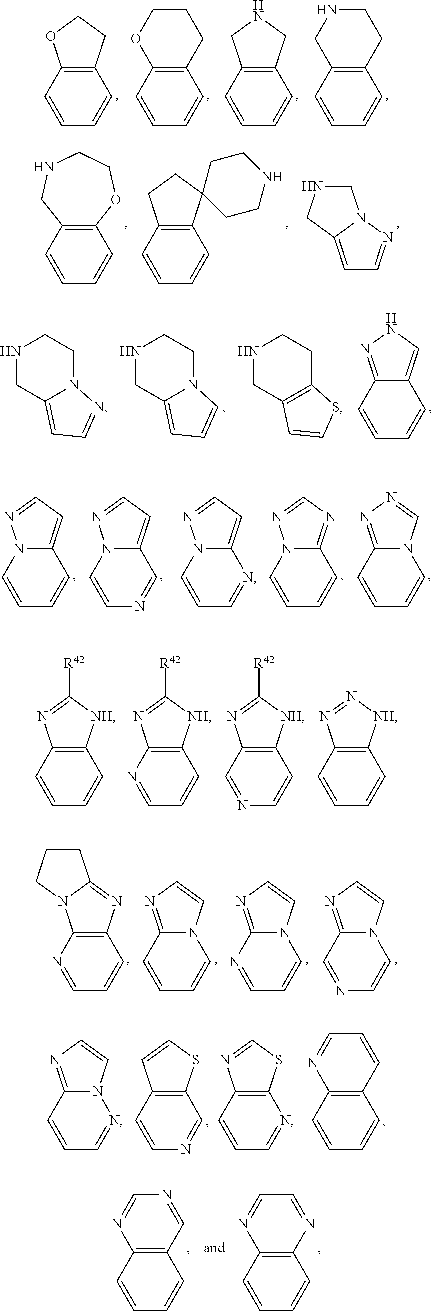

and optionally substituted with 1-10 (e.g., 1-9, 1-8, 1-7, 1-6, 1-5, 1-4, 1-3, 1-2, 1) R⁶.

In some embodiments of Formula Ie, R¹ is selected from the heteroaryl group consisting of:

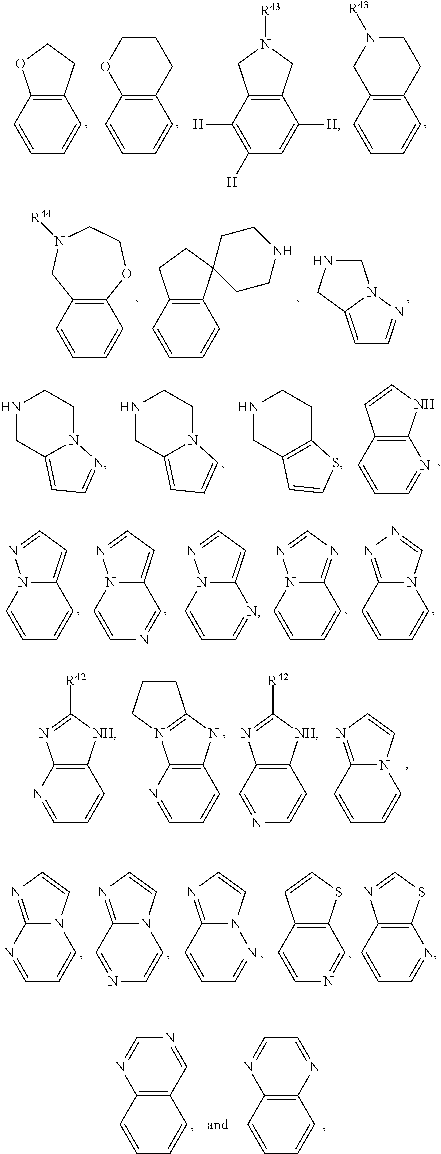

and optionally substituted with 1-10 (e.g., 1-9, 1-8, 1-7, 1-6, 1-5, 1-4, 1-3, 1-2, 1) R⁶; and

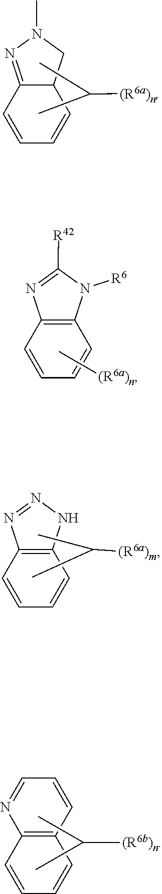

and

In some embodiments of Formula Ie, there is the proviso that when R¹ is

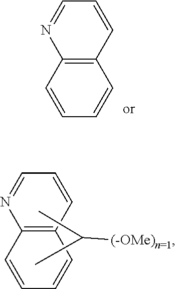

R³ is not halide or methyl.

In some embodiments of Formulas Ia, Ib, and Ie, R¹ is selected from the group consisting of:

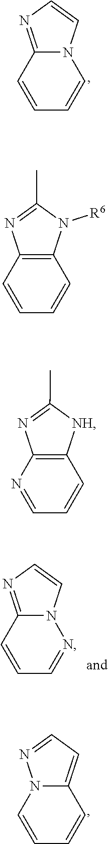

optionally substituted with 1-2 R⁶.

In some embodiments of Formulas Ia, Ib, and Ie, R¹ is selected from the group consisting of:

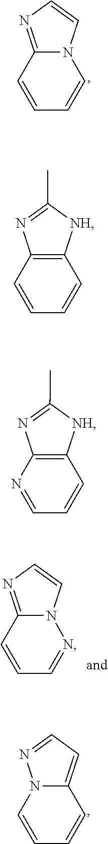

optionally substituted with 1-2 R⁶.

In some embodiments of Formula I, a carbon atom on an aromatic ring of the heteroaryl R¹ group form the bond with

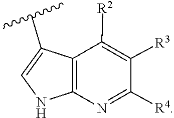

In some embodiments of Formula Ia, a carbon atom on an aromatic ring of the heteroaryl R¹ group form the bond with

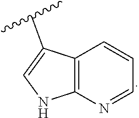 [SMILES: *c1c[nH]c2ncccc12]

In some embodiments of Formula Ib, a carbon atom on an aromatic ring of the heteroaryl R¹ group form the bond with

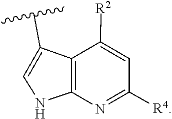

In some embodiments of Formula Ic, a carbon atom on an aromatic ring of the heteroaryl R¹ group form the bond with

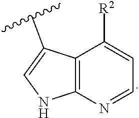

In some embodiments of Formula Id, a carbon atom on an aromatic ring of the heteroaryl R¹ group form the bond with

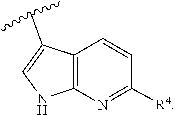

In some embodiments of Formula Ie, a carbon atom on an aromatic ring of the heteroaryl R¹ group form the bond with

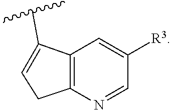

In some embodiments of Formulas I, Ib, and Ic, R² is selected from the group consisting of H, —OR⁷, and 5-membered heteroaryl optionally substituted with 1-3 R⁸, and —NHR⁹.

In some embodiments of Formulas I and Ie, R³ is selected from the group consisting of H, halide (e.g., F, Cl, Br, I), CN, —CO₂H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚheterocyclyl optionally substituted with 1-10 (e.g., 1-9, 1-8, 1-7, 1-6, 1-5, 1-4, 1-3, 1-2, 1) R¹¹, -(5-9 membered heteroaryl) optionally substituted with 1-10 (e.g., 1-9, 1-8, 1-7, 1-6, 1-5, 1-4, 1-3, 1-2, 1) R¹²; phenyl optionally substituted with 1-5 (e.g., 1-4, 1-3, 1-2, 1) R¹³, —NHheteroaryl optionally substituted with 1-10 (e.g., 1-9, 1-8, 1-7, 1-6, 1-5, 1-4, 1-3, 1-2, 1) R¹⁴; —C(═O)NHR¹⁵, —NH(C═O)R¹⁶, and —C(═O)R¹⁷.

In some embodiments of Formulas I, Ib, and Ie, R³ and R⁴ are H and R² is selected from the group consisting of —OMe and a 5-membered heteroaryl optionally substituted with 1 R⁸.

In some embodiments of Formulas I, Ib, and Ie, R⁴ is H and R² is selected from the group consisting of —OMe and a 5-membered heteroaryl optionally substituted with 1 R⁸.

In some embodiments of Formulas I and Ib, R² and R⁴ are H and R³ is selected from the group consisting of -heterocyclyl optionally substituted with 1 R¹¹, -(5-6 membered heteroaryl) optionally substituted with 1-2 R¹²; —C(═O)NHR¹⁵, —NH(C═O)R¹⁶, and —C(═O)R¹⁷.

In some embodiments of Formula Ie, R³ is selected from the group consisting of -heterocyclyl optionally substituted with 1 R¹¹, -(5-6 membered heteroaryl) optionally substituted with 1-2 R¹²; —C(═O)NHR¹⁵, —NH(C═O)R¹⁶, and —C(═O)R¹⁷.

In some embodiments of Formula I, R³ is selected from the group consisting of H, CN, —CO₂H, —OR¹⁰, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚheterocyclyl optionally substituted with 1-10 (e.g., 1-9, 1-8, 1-7, 1-6, 1-5, 1-4, 1-3, 1-2, 1) R¹¹, -(5-9 membered heteroaryl) optionally substituted with 1-10 (e.g., 1-9, 1-8, 1-7, 1-6, 1-5, 1-4, 1-3, 1-2, 1) R¹²; phenyl optionally substituted with 1-5 R¹³, —NHheteroaryl optionally substituted with 1-10 (e.g., 1-9, 1-8, 1-7, 1-6, 1-5, 1-4, 1-3, 1-2, 1) R¹⁴; —C(═O)NHR¹⁵, —NH(C═O)R¹⁶, and —C(═O)R¹⁷.

In some embodiments of Formulas I, Ib, and Id, R⁴ is selected from the group consisting of H, halide (e.g., F, Cl, Br, I), CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 (e.g., 1-9, 1-8, 1-7, 1-6, 1-5, 1-4, 1-3, 1-2, 1) R¹⁹; phenyl optionally substituted with 1-5 (e.g., 1-4, 1-3, 1-2, 1) R²⁰, —NHphenyl optionally substituted with 1-5 (e.g., 1-4, 1-3, 1-2, 1) R²¹, —(CH₂)ₚcarbocyclyl optionally substituted with 1-12 (e.g., 1-11, 1-10, 1-9, 1-8, 1-7, 1-6, 1-5, 1-4, 1-3, 1-2, 1) R²², —C(═O)NHR²³, —NH(C═O)R²⁴, and —C(═O)R²⁵.

In some embodiments of Formulas I, Ib, and Ie, R² and R³ are H and R⁴ is selected from the group consisting of -carbocyclyl, —C(═O)NHR²³, and —NH(C═O)R²⁴.

In some embodiments of Formulas I, Ib, and Ie, R² is H and R⁴ is selected from the group consisting of -carbocyclyl, —C(═O)NHR²³, and —NH(C═O)R²⁴.

In some embodiments of Formula I, there is the proviso that at least two of R², R³, and R⁴ are H.

In some embodiments of Formula Ib, there is the proviso that one of R² and R⁴ is H and the other of R² and R⁴ is not H.

In some embodiments of Formula I, each R⁵ is independently selected from the group consisting of —OR²⁶, -heterocyclyl optionally substituted with 1-10 R²⁷, and —NHheterocyclyl optionally substituted with 1-10 (e.g., 1-9, 1-8, 1-7, 1-6, 1-5, 1-4, 1-3, 1-2, 1) R²⁸.

In some embodiments of Formula I, each R⁵ is independently selected from the group consisting of —OMe and -heterocyclyl optionally substituted with 1 R²⁷.

In some embodiments of Formulas I, Ia, Ib, Ic, Id, and Ie, each R⁶ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 (e.g., 1-9, 1-8, 1-7, 1-6, 1-5, 1-4, 1-3, 1-2, 1) R³⁰, —NHheterocyclyl optionally substituted with 1-10 (e.g., 1-9, 1-8, 1-7, 1-6, 1-5, 1-4, 1-3, 1-2, 1) R³¹, -heteroaryl optionally substituted with 1-10 (e.g., 1-9, 1-8, 1-7, 1-6, 1-5, 1-4, 1-3, 1-2, 1) R³²; —NHheteroaryl optionally substituted with 1-10 R³³; —NHCH₂heteroaryl optionally substituted with 1-10 (e.g., 1-9, 1-8, 1-7, 1-6, 1-5, 1-4, 1-3, 1-2, 1) R³³; ⁻CH₂N(R³⁴)₂, —C(═O)N(R³⁵)₂, and —C(═O)R³⁶.

In some embodiments of Formulas I, Ia, Ib, Ic, Id, and Ie, two R⁶ attached to the same carbon atom are taken together to form a carbonyl group.

In some embodiments of Formulas I, Ia, Ib, Ic, Id, and Ie, two R⁶ attached to the same carbon atom are taken together to form a ring which is selected from the group consisting of -heterocyclyl optionally substituted with 1-10 (e.g., 1-9, 1-8, 1-7, 1-6, 1-5, 1-4, 1-3, 1-2, 1) R³⁷ and -carbocyclyl optionally substituted with 1-12 (e.g., 1-11, 1-10, 1-9, 1-8, 1-7, 1-6, 1-5, 1-4, 1-3, 1-2, 1) R³⁸.

In some embodiments of Formulas I, Ia, Ib, Ic, Id, and Ie, each R⁶ is independently selected from the group consisting of unsubstituted —(C₁₋₃ alkyl), -heterocyclyl optionally substituted with 1 R³⁰, -(5-6 membered heteroaryl) optionally substituted with 1-2 R³², and —C(═O)NHR³⁵.

In some embodiments of Formula Ia, each R^{6a} is independently selected from the group consisting of —OR^{29a}, —CH₂OH, -heterocyclyl optionally substituted with 1-10 (e.g., 1-9, 1-8, 1-7, 1-6, 1-5, 1-4, 1-3, 1-2, 1) R³⁰, —CH₂N(R³⁴)₂, —C(═O)N(R³⁵)₂, and —C(═O)R³⁶.

In some embodiments of Formula Ic, each R^{6a} is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 (e.g., 1-9, 1-8, 1-7, 1-6, 1-5, 1-4, 1-3, 1-2, 1) R³⁰, —NHheterocyclyl optionally substituted with 1-10 (e.g., 1-9, 1-8, 1-7, 1-6, 1-5, 1-4, 1-3, 1-2, 1) R³¹, -heteroaryl optionally substituted with 1-10 R³²; —NHheteroaryl optionally substituted with 1-10 (e.g., 1-9, 1-8, 1-7, 1-6, 1-5, 1-4, 1-3, 1-2, 1) R³³; —NHCH₂heteroaryl optionally substituted with 1-10 (e.g., 1-9, 1-8, 1-7, 1-6, 1-5, 1-4, 1-3, 1-2, 1) R³³; —CH₂N(R³⁴)₂, —C(═O)N(R³⁵)₂, and —C(═O)R³⁶.

In some embodiments of Formula Ie, each R^{6a} is independently selected from the group consisting of halide (e.g., F, Cl, Br, I), unsubstituted —(C₂₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 (e.g., 1-9, 1-8, 1-7, 1-6, 1-5, 1-4, 1-3, 1-2, 1) R³⁰, —NHheterocyclyl optionally substituted with 1-10 (e.g., 1-9, 1-8, 1-7, 1-6, 1-5, 1-4, 1-3, 1-2, 1) R³¹, -heteroaryl optionally substituted with 1-10 (e.g., 1-9, 1-8, 1-7, 1-6, 1-5, 1-4, 1-3, 1-2, 1) R³²; —NHheteroaryl optionally substituted with 1-10 (e.g., 1-9, 1-8, 1-7, 1-6, 1-5, 1-4, 1-3, 1-2, 1) R³³; —NHCH₂heteroaryl optionally substituted with 1-10 (e.g., 1-9, 1-8, 1-7, 1-6, 1-5, 1-4, 1-3, 1-2, 1) R³³; —CH₂N(R³⁴)₂, —C(═O)N(R³⁵)₂, and —C(═O)R³⁶.

In some embodiments of Formula Ie, each R^{6b} is independently selected from the group consisting of —OR²⁹, —CH₂OH, -heterocyclyl optionally substituted with 1-10 (e.g., 1-9, 1-8, 1-7, 1-6, 1-5, 1-4, 1-3, 1-2, 1) R³⁰, —CH₂N(R³⁴)₂, —C(═O)N(R³⁵)₂, and —C(═O)R³⁶.

In some embodiments of Formulas I, Ib, and Ic, R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ib, and Ic, each R⁸ is independently selected from the group consisting of H, halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ib, and Ic, R⁹ is —CH₂(6-membered heteroaryl) optionally substituted with 1-4 R³⁹.

In some embodiments of Formulas I and Ie, R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ie, each R¹¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I and Ie, each R¹² is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formulas I and Ie, each R¹³ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formulas I and Ie, each R¹⁴ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formulas I and Ie, R¹⁵ is selected from the group consisting of -heterocyclyl optionally substituted with 1-10 (e.g., 1-9, 1-8, 1-7, 1-6, 1-5, 1-4, 1-3, 1-2, 1) R³⁷ and -heteroaryl optionally substituted with 1-10 (e.g., 1-9, 1-8, 1-7, 1-6, 1-5, 1-4, 1-3, 1-2, 1) R³⁹.

In some embodiments of Formulas I and Ie, R¹⁶ is selected from the group consisting of -heterocyclyl optionally substituted with 1-10 (e.g., 1-9, 1-8, 1-7, 1-6, 1-5, 1-4, 1-3, 1-2, 1) R³⁷ and -heteroaryl optionally substituted with 1-10 (e.g., 1-9, 1-8, 1-7, 1-6, 1-5, 1-4, 1-3, 1-2, 1) R³⁹.

In some embodiments of Formulas I and Ie, R¹⁷ is selected from the group consisting of -heterocyclyl optionally substituted with 1-10 (e.g., 1-9, 1-8, 1-7, 1-6, 1-5, 1-4, 1-3, 1-2, 1) R³⁷ and -heteroaryl optionally substituted with 1-10 (e.g., 1-9, 1-8, 1-7, 1-6, 1-5, 1-4, 1-3, 1-2, 1) R³⁹.

In some embodiments of Formulas I, Ib, and Id, R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 (e.g., 1-11, 1-10, 1-9, 1-8, 1-7, 1-6, 1-5, 1-4, 1-3, 1-2, 1) R³⁸.

In some embodiments of Formulas I, Ib, and Id, each R¹⁹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ib, and Id, each R²⁰ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ib, and Id, each R²¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formulas I, Ib, and Id, each R²² is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ib, and Id, R²³ is selected from the group consisting of -heterocyclyl optionally substituted with 1-10 (e.g., 1-9, 1-8, 1-7, 1-6, 1-5, 1-4, 1-3, 1-2, 1) R³⁷ and -heteroaryl optionally substituted with 1-10 (e.g., 1-9, 1-8, 1-7, 1-6, 1-5, 1-4, 1-3, 1-2, 1) R³⁹.

In some embodiments of Formulas I, Ib, and Id, R²⁴ is selected from the group consisting of -heterocyclyl optionally substituted with 1-10 (e.g., 1-9, 1-8, 1-7, 1-6, 1-5, 1-4, 1-3, 1-2, 1) R³⁷ and -heteroaryl optionally substituted with 1-10 (e.g., 1-9, 1-8, 1-7, 1-6, 1-5, 1-4, 1-3, 1-2, 1) R³⁹.

In some embodiments of Formulas I, Ib, and Id, R²⁵ is selected from the group consisting of -heterocyclyl optionally substituted with 1-10 (e.g., 1-9, 1-8, 1-7, 1-6, 1-5, 1-4, 1-3, 1-2, 1) R³⁷ and -heteroaryl optionally substituted with 1-10 (e.g., 1-9, 1-8, 1-7, 1-6, 1-5, 1-4, 1-3, 1-2, 1) R³⁹.

In some embodiments of Formula I, each R²⁶ is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R²⁶ is unsubstituted —(C₁₋₉ haloalkyl).

In some embodiments of Formula I, each R²⁷ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, each R²⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formula Ia, each R^{29a} is -heterocyclyl optionally substituted with 1-10 (e.g., 1-9, 1-8, 1-7, 1-6, 1-5, 1-4, 1-3, 1-2, 1) R³⁷.

In some embodiments of Formulas I, Ia, Ib, Ic, Id, and Ie, each R³⁰ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, two R³⁰ attached to the same carbon atom are taken together to form a ring which is selected from the group consisting of -heterocyclyl optionally substituted with 1-10 (e.g., 1-9, 1-8, 1-7, 1-6, 1-5, 1-4, 1-3, 1-2, 1) R³⁷ and -carbocyclyl optionally substituted with 1-12 (e.g., 1-11, 1-10, 1-9, 1-8, 1-7, 1-6, 1-5, 1-4, 1-3, 1-2, 1) R³⁸.

In some embodiments of Formulas I, Ia, Ib, Ic, Id, and Ie, each R³¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, each R³² is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, each R³³ is independently selected from the group consisting of halide (e.g., F, Cl, Br, I), —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formulas I, Ia, Ib, Ic, Id, and Ie, each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 (e.g., 1-9, 1-8, 1-7, 1-6, 1-5, 1-4, 1-3, 1-2, 1) R⁴⁰, and -heteroaryl optionally substituted with 1-10 (e.g., 1-9, 1-8, 1-7, 1-6, 1-5, 1-4, 1-3, 1-2, 1) R³⁹.

In some embodiments of Formulas I, Ia, Ib, Ic, Id, and Ie, each R³⁶ is -heterocyclyl optionally substituted with 1-10 (e.g., 1-9, 1-8, 1-7, 1-6, 1-5, 1-4, 1-3, 1-2, 1) R⁴¹.

In some embodiments of Formulas I, Ia, Ib, Ic, Id, and Ie, each R³⁷ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, each R³⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formulas I, Ia, Ib, Ic, Id, and Ie, each R³⁹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 (e.g., 1-9, 1-8, 1-7, 1-6, 1-5, 1-4, 1-3, 1-2, 1) R³⁷, and -heteroaryl optionally substituted with 1-10 (e.g., 1-9, 1-8, 1-7, 1-6, 1-5, 1-4, 1-3, 1-2, 1) R³⁸.

In some embodiments of Formulas I, Ia, Ib, Ic, Id, and Ie, each R⁴⁰ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 (e.g., 1-9, 1-8, 1-7, 1-6, 1-5, 1-4, 1-3, 1-2, 1) R³⁸, and —(CH₂)ₚheteroaryl optionally substituted with 1-10 (e.g., 1-9, 1-8, 1-7, 1-6, 1-5, 1-4, 1-3, 1-2, 1) R³⁹.

In some embodiments of Formulas I, Ia, Ib, Ic, Id, and Ie, each R⁴¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 (e.g., 1-11, 1-10, 1-9, 1-8, 1-7, 1-6, 1-5, 1-4, 1-3, 1-2, 1) R³⁸, and -heterocyclyl optionally substituted with 1-10 (e.g., 1-9, 1-8, 1-7, 1-6, 1-5, 1-4, 1-3, 1-2, 1) R³⁷.

In some embodiments of Formulas I, Ia, Ib, Ic, Id, and Ie, two R⁴¹ attached to the same carbon atom are taken together to form a ring which is selected from the group consisting of -heterocyclyl optionally substituted with 1-10 (e.g., 1-9, 1-8, 1-7, 1-6, 1-5, 1-4, 1-3, 1-2, 1) R³⁷ and -carbocyclyl optionally substituted with 1-12 (e.g., 1-11, 1-10, 1-9, 1-8, 1-7, 1-6, 1-5, 1-4, 1-3, 1-2, 1) R³⁸.

In some embodiments of Formulas Ia, Ib, Id, and Ie, each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula Ie, each R⁴³ is H.

In some embodiments of Formula Ie, each R⁴⁴ is independently selected from the group consisting of H, unsubstituted —(C₂₋₉ alkyl), unsubstituted —(C₂₋₉ alkenyl), unsubstituted —(C₂₋₉ alkynyl), and unsubstituted —(Cl-9 haloalkyl).

In some embodiments of Formulas I, Ia, Ib, Ic, Id, and Ie, each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 (e.g., 1-9, 1-8, 1-7, 1-6, 1-5, 1-4, 1-3, 1-2, 1) R³⁷.

In some embodiments of Formula Ia, each n is independently 0 or 3.

In some embodiments of Formula Ie, each n is independently 0 or 6.

In some embodiments of Formulas I, Ia, Ib, Ic, Id, and Ie, each m is independently 1 or 6.

In some embodiments of Formulas I, Ia, Ib, Ic, Id, and Ie, each p is independently 0 or 1.

In some embodiments, there is the proviso that Formula I is not a structure selected from the group consisting of:

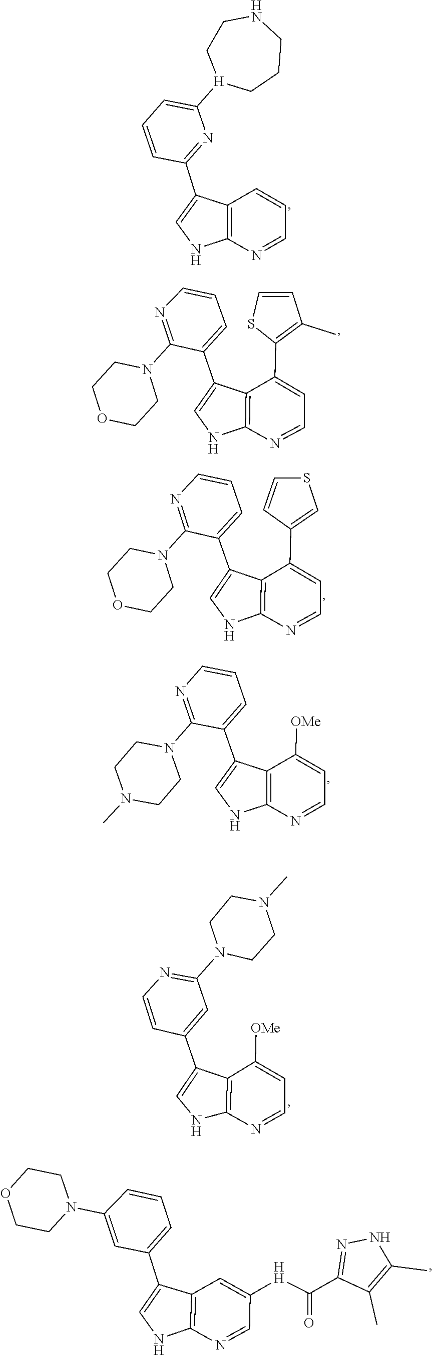

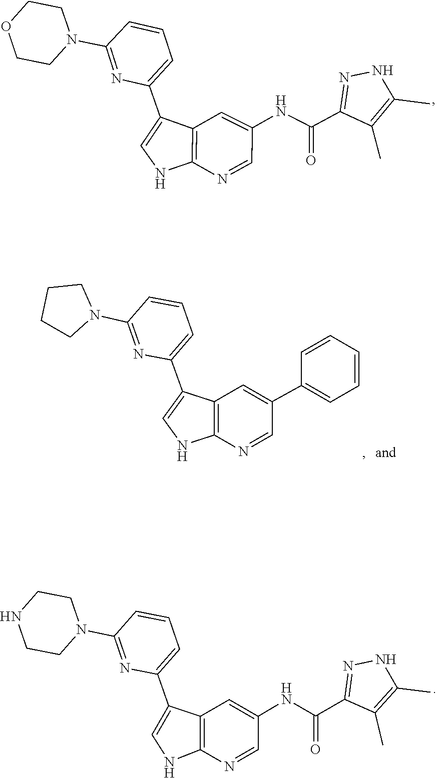 [SMILES: Cc1[nH]nc(C(=O)Nc2cnc3[nH]cc(-c4cccc(N5CCOCC5)n4)c3c2)c1C ; c1ccc(-c2cnc3[nH]cc(-c4cccc(N5CCCC5)n4)c3c2)cc1 ; Cc1[nH]nc(C(=O)Nc2cnc3[nH]cc(-c4cccc(N5CCNCC5)n4)c3c2)c1C]

In some embodiments, there is the proviso that Formula Ia is not a structure selected from the group consisting of:

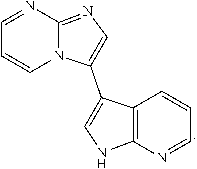 [SMILES: c1cnc2[nH]cc(-c3cnc4ncccn34)c2c1]

In some embodiments, there is the proviso that Formula Ib is not a structure selected from the group consisting of:

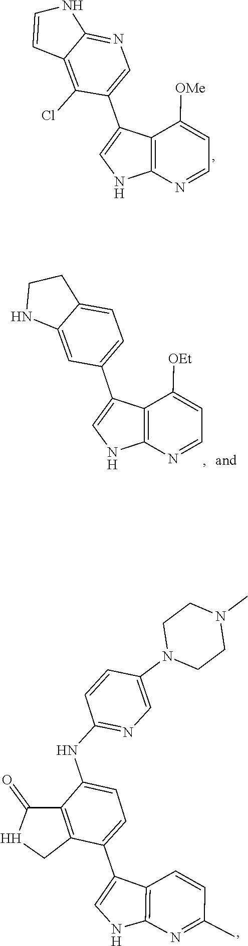

In some embodiments, there is the proviso that Formula Ic is not a structure selected from the group consisting of:

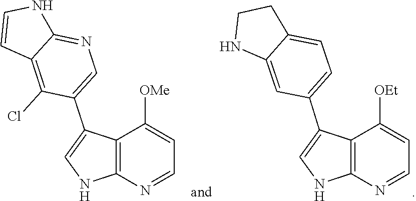 [SMILES: COc1ccnc2[nH]cc(-c3cnc4[nH]ccc4c3Cl)c12 ; CCOc1ccnc2[nH]cc(-c3ccc4c(c3)NCC4)c12]

In some embodiments, there is the proviso that Formula Id is not a structure selected from the group consisting of:

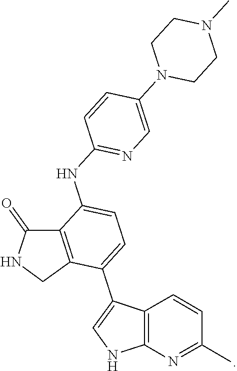 [SMILES: Cc1ccc2c(-c3ccc(Nc4ccc(N5CCN(C)CC5)cn4)c4c3CNC4=O)c[nH]c2n1]

In some embodiments, there is the proviso that Formula Ie is not a structure selected from the group consisting of:

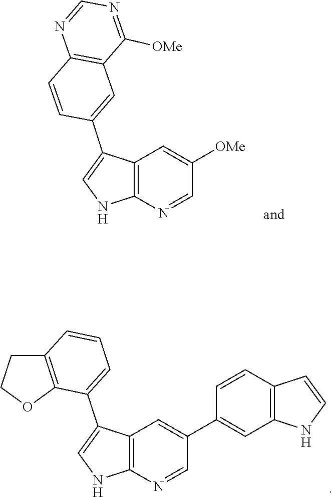 [SMILES: COc1cnc2[nH]cc(-c3ccc4ncnc(OC)c4c3)c2c1 ; c1cc2c(c(-c3c[nH]c4ncc(-c5ccc6cc[nH]c6c5)cc34)c1)OCC2]

In some embodiments, each m is independently 1 to 6 (e.g., 1-5, 1-4, 1-3, 1-2, 1).

In some embodiments, each n is independently 0 to 6 (e.g., 0-5, 0-4, 0-3, 0-2, 0-1, 0).

Illustrative compounds of Formulas I, Ia, Ib, Ic, Id, and Ie, are shown in Table 1.

TABLE 1

| | |
|---|---|
| 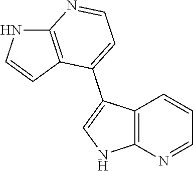 [SMILES: c1cnc2[nH]cc(-c3ccnc4[nH]ccc34)c2c1] | 2 |
| 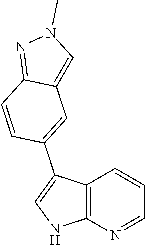 [SMILES: Cn1cc2cc(-c3c[nH]c4ncccc34)ccc2n1] | 3 |
| 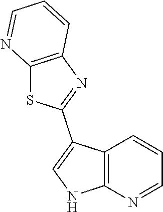 [SMILES: c1cnc2sc(-c3c[nH]c4ncccc34)nc2c1] | 4 |
| 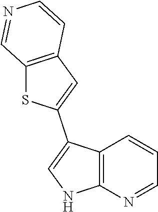 [SMILES: c1cnc2[nH]cc(-c3cc4ccncc4s3)c2c1] | 5 |
| 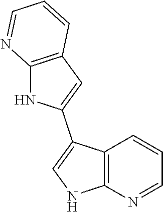 [SMILES: c1cnc2[nH]c(-c3c[nH]c4ncccc34)cc2c1] | 6 |
| 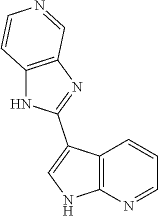 [SMILES: c1cnc2[nH]cc(-c3nc4cnccc4[nH]3)c2c1] | 7 |
| 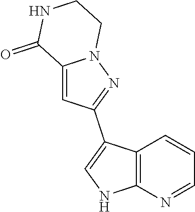 [SMILES: O=C1NCCn2nc(-c3c[nH]c4ncccc34)cc21] | 8 |
| 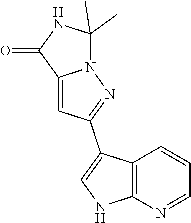 [SMILES: CC1(C)NC(=O)c2cc(-c3c[nH]c4ncccc34)nn21] | 9 |
| 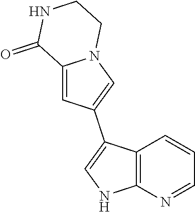 [SMILES: O=C1NCCn2cc(-c3c[nH]c4ncccc34)cc21] | 10 |
| 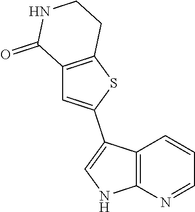 [SMILES: O=C1NCCc2sc(-c3c[nH]c4ncccc34)cc21] | 11 |
| 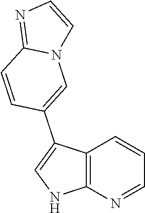 [SMILES: c1cnc2[nH]cc(-c3ccc4nccn4c3)c2c1] | 12 |
| 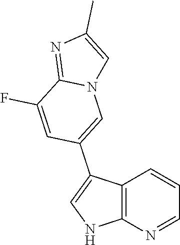 [SMILES: Cc1cn2cc(-c3c[nH]c4ncccc34)cc(F)c2n1] | 13 |
| 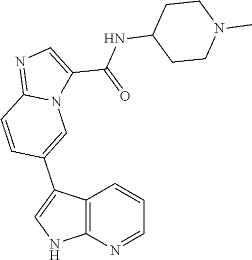 [SMILES: CN1CCC(NC(=O)c2cnc3ccc(-c4c[nH]c5ncccc45)cn23)CC1] | 14 |
| 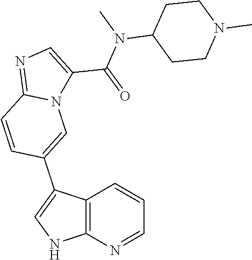 [SMILES: CN1CCC(N(C)C(=O)c2cnc3ccc(-c4c[nH]c5ncccc45)cn23)CC1] | 15 |
| 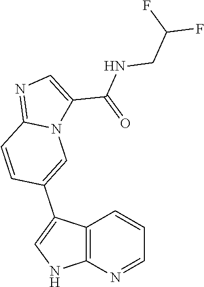 [SMILES: O=C(NCC(F)F)c1cnc2ccc(-c3c[nH]c4ncccc34)cn12] | 16 |
| 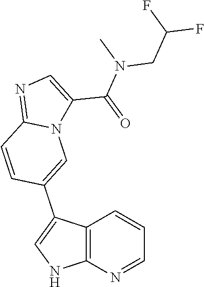 [SMILES: CN(CC(F)F)C(=O)c1cnc2ccc(-c3c[nH]c4ncccc34)cn12] | 17 |
| 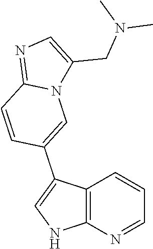 [SMILES: CN(C)Cc1cnc2ccc(-c3c[nH]c4ncccc34)cn12] | 18 |
| 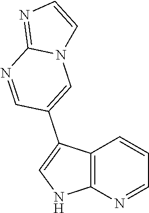 [SMILES: c1cnc2[nH]cc(-c3cnc4nccn4c3)c2c1] | 19 |
| 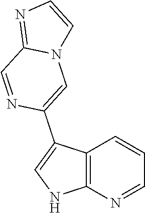 [SMILES: c1cnc2[nH]cc(-c3cn4ccnc4cn3)c2c1] | 20 |
| 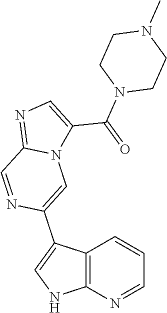 [SMILES: CN1CCN(C(=O)c2cnc3cnc(-c4c[nH]c5ncccc45)cn23)CC1] | 21 |
| 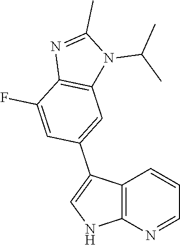 [SMILES: Cc1nc2c(F)cc(-c3c[nH]c4ncccc34)cc2n1C(C)C] | 22 |
| 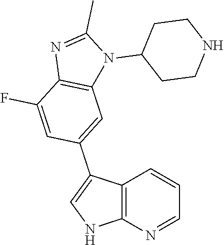 [SMILES: Cc1nc2c(F)cc(-c3c[nH]c4ncccc34)cc2n1C1CCNCC1] | 23 |
| 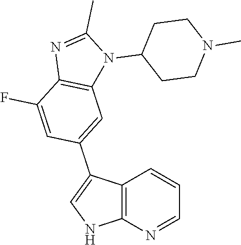 [SMILES: Cc1nc2c(F)cc(-c3c[nH]c4ncccc34)cc2n1C1CCN(C)CC1] | 24 |
| 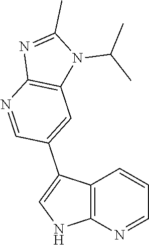 [SMILES: Cc1nc2ncc(-c3c[nH]c4ncccc34)cc2n1C(C)C] | 25 |
| 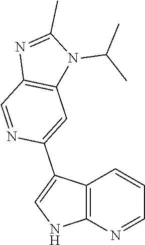 [SMILES: Cc1nc2cnc(-c3c[nH]c4ncccc34)cc2n1C(C)C] | 26 |
| 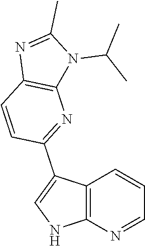 [SMILES: Cc1nc2ccc(-c3c[nH]c4ncccc34)nc2n1C(C)C] | 27 |
| 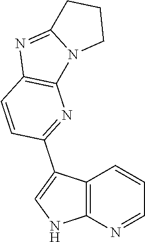 [SMILES: c1cnc2[nH]cc(-c3ccc4nc5n(c4n3)CCC5)c2c1] | 28 |
| 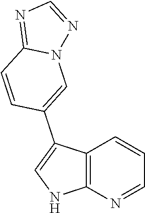 [SMILES: c1cnc2[nH]cc(-c3ccc4ncnn4c3)c2c1] | 29 |
| 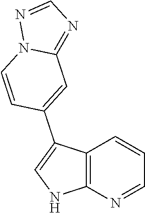 [SMILES: c1cnc2[nH]cc(-c3ccn4ncnc4c3)c2c1] | 30 |
| 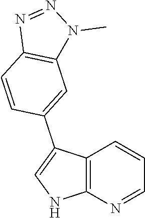 [SMILES: Cn1nnc2ccc(-c3c[nH]c4ncccc34)cc21] | 31 |
| 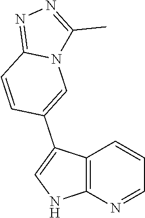 [SMILES: Cc1nnc2ccc(-c3c[nH]c4ncccc34)cn12] | 32 |
| 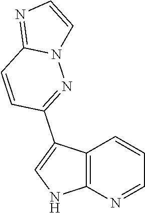 [SMILES: c1cnc2[nH]cc(-c3ccc4nccn4n3)c2c1] | 33 |
| 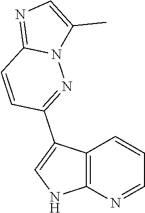 [SMILES: Cc1cnc2ccc(-c3c[nH]c4ncccc34)nn12] | 34 |
| 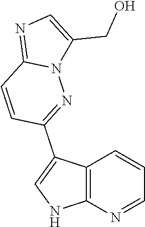 [SMILES: OCc1cnc2ccc(-c3c[nH]c4ncccc34)nn12] | 35 |
| 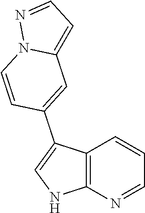 [SMILES: c1cnc2[nH]cc(-c3ccn4nccc4c3)c2c1] | 36 |
| 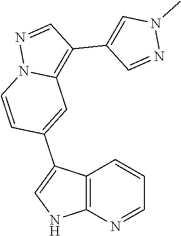 [SMILES: Cn1cc(-c2cnn3ccc(-c4c[nH]c5ncccc45)cc23)cn1] | 37 |
| 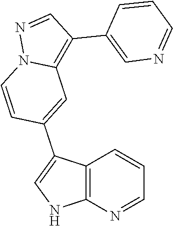 [SMILES: c1cncc(-c2cnn3ccc(-c4c[nH]c5ncccc45)cc23)c1] | 38 |
| 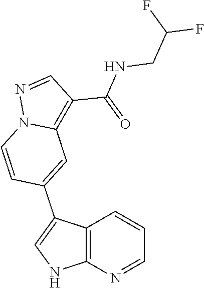 [SMILES: O=C(NCC(F)F)c1cnn2ccc(-c3c[nH]c4ncccc34)cc12] | 39 |
| 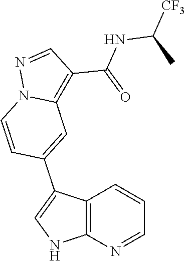 [SMILES: C[C@@H](NC(=O)c1cnn2ccc(-c3c[nH]c4ncccc34)cc12)C(F)(F)F] | 40 |
| 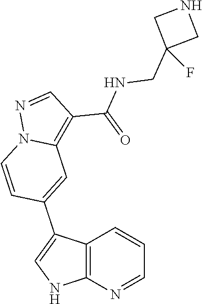 [SMILES: O=C(NCC1(F)CNC1)c1cnn2ccc(-c3c[nH]c4ncccc34)cc12] | 41 |
| 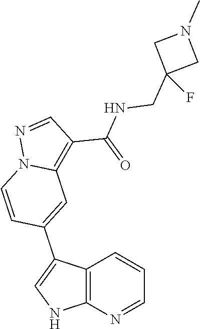 [SMILES: CN1CC(F)(CNC(=O)c2cnn3ccc(-c4c[nH]c5ncccc45)cc23)C1] | 42 |
| 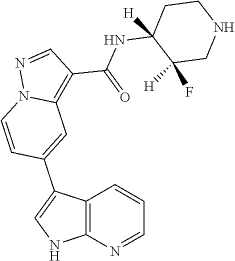 [SMILES: O=C(N[C@@H]1CCNC[C@H]1F)c1cnn2ccc(-c3c[nH]c4ncccc34)cc12] | 43 |
| 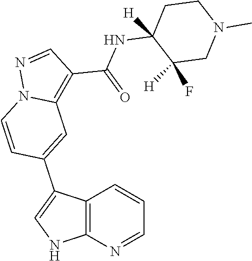 [SMILES: CN1CC[C@@H](NC(=O)c2cnn3ccc(-c4c[nH]c5ncccc45)cc23)[C@H](F)C1] | 44 |
| 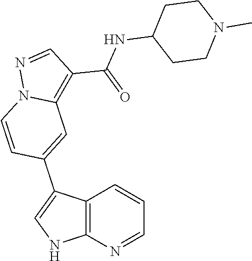 [SMILES: CN1CCC(NC(=O)c2cnn3ccc(-c4c[nH]c5ncccc45)cc23)CC1] | 45 |
| 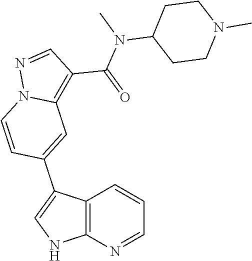 [SMILES: CN1CCC(N(C)C(=O)c2cnn3ccc(-c4c[nH]c5ncccc45)cc23)CC1] | 46 |
| 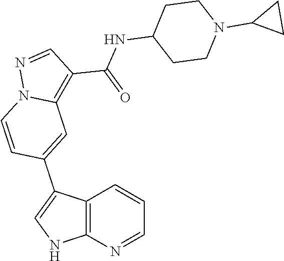 [SMILES: O=C(NC1CCN(C2CC2)CC1)c1cnn2ccc(-c3c[nH]c4ncccc34)cc12] | 47 |
| 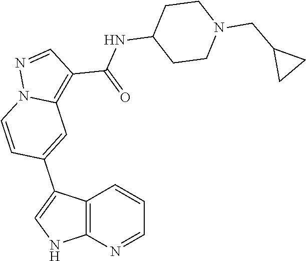 [SMILES: O=C(NC1CCN(CC2CC2)CC1)c1cnn2ccc(-c3c[nH]c4ncccc34)cc12] | 48 |
| 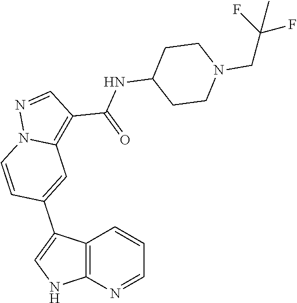 [SMILES: CC(F)(F)CN1CCC(NC(=O)c2cnn3ccc(-c4c[nH]c5ncccc45)cc23)CC1] | 49 |
| 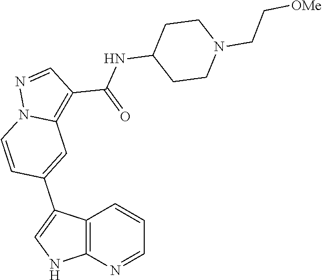 [SMILES: COCCN1CCC(NC(=O)c2cnn3ccc(-c4c[nH]c5ncccc45)cc23)CC1] | 50 |
| 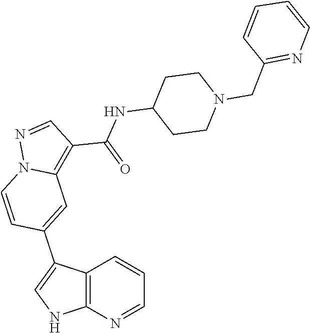 [SMILES: O=C(NC1CCN(Cc2ccccn2)CC1)c1cnn2ccc(-c3c[nH]c4ncccc34)cc12] | 51 |
| 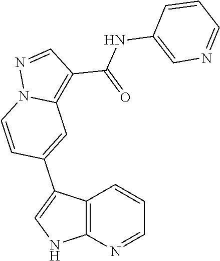 [SMILES: O=C(Nc1cccnc1)c1cnn2ccc(-c3c[nH]c4ncccc34)cc12] | 52 |
| 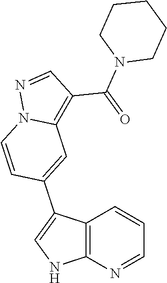 [SMILES: O=C(c1cnn2ccc(-c3c[nH]c4ncccc34)cc12)N1CCCCC1] | 53 |
| 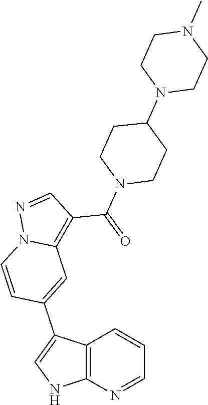 [SMILES: CN1CCN(C2CCN(C(=O)c3cnn4ccc(-c5c[nH]c6ncccc56)cc34)CC2)CC1] | 54 |
| 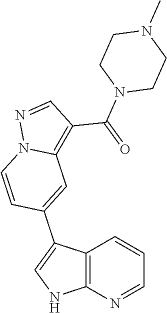 [SMILES: CN1CCN(C(=O)c2cnn3ccc(-c4c[nH]c5ncccc45)cc23)CC1] | 55 |
| 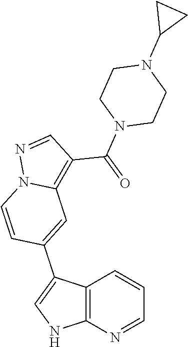 [SMILES: O=C(c1cnn2ccc(-c3c[nH]c4ncccc34)cc12)N1CCN(C2CC2)CC1] | 56 |
| 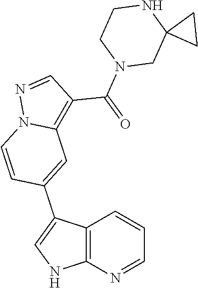 [SMILES: O=C(c1cnn2ccc(-c3c[nH]c4ncccc34)cc12)N1CCNC2(CC2)C1] | 57 |
| 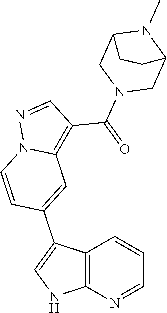 [SMILES: CN1C2CCC1CN(C(=O)c1cnn3ccc(-c4c[nH]c5ncccc45)cc13)C2] | 58 |
| 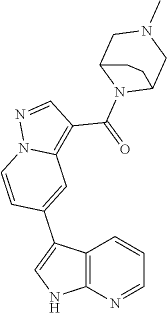 [SMILES: CN1CC2CCC(C1)N2C(=O)c1cnn2ccc(-c3c[nH]c4ncccc34)cc12] | 59 |
| 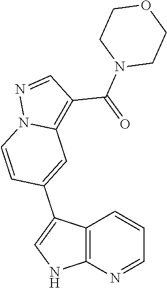 [SMILES: O=C(c1cnn2ccc(-c3c[nH]c4ncccc34)cc12)N1CCOCC1] | 60 |
| 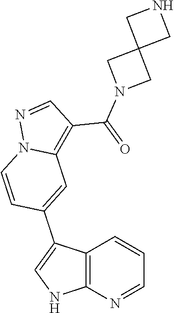 [SMILES: O=C(c1cnn2ccc(-c3c[nH]c4ncccc34)cc12)N1CC2(CNC2)C1] | 61 |
| 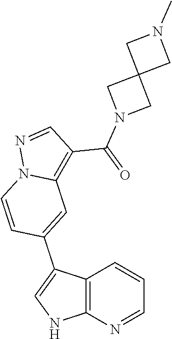 [SMILES: CN1CC2(C1)CN(C(=O)c1cnn3ccc(-c4c[nH]c5ncccc45)cc13)C2] | 62 |
| 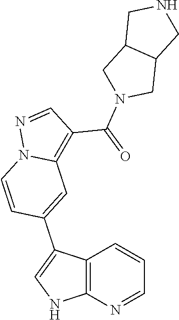 [SMILES: O=C(c1cnn2ccc(-c3c[nH]c4ncccc34)cc12)N1CC2CNCC2C1] | 63 |
| 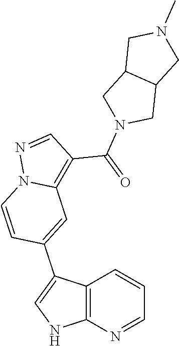 [SMILES: CN1CC2CN(C(=O)c3cnn4ccc(-c5c[nH]c6ncccc56)cc34)CC2C1] | 64 |
| 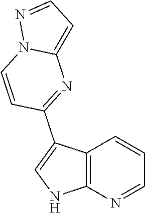 [SMILES: c1cnc2[nH]cc(-c3ccn4nccc4n3)c2c1] | 65 |
| 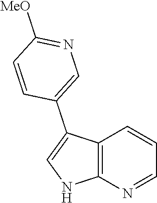 [SMILES: COc1ccc(-c2c[nH]c3ncccc23)cn1] | 67 |
| 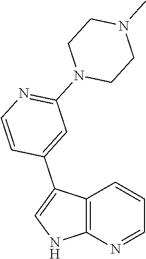 [SMILES: CN1CCN(c2cc(-c3c[nH]c4ncccc34)ccn2)CC1] | 68 |
| 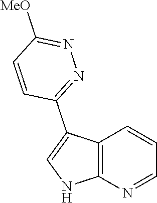 [SMILES: COc1ccc(-c2c[nH]c3ncccc23)nn1] | 69 |
| 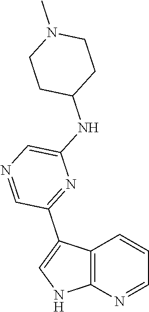 [SMILES: CN1CCC(Nc2cncc(-c3c[nH]c4ncccc34)n2)CC1] | 70 |
| 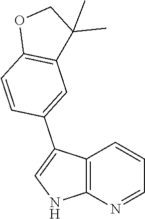 [SMILES: CC1(C)COc2ccc(-c3c[nH]c4ncccc34)cc21] | 72 |
| 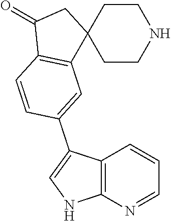 [SMILES: O=C1CC2(CCNCC2)c2cc(-c3c[nH]c4ncccc34)ccc21] | 73 |
| 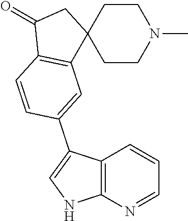 [SMILES: CN1CCC2(CC1)CC(=O)c1ccc(-c3c[nH]c4ncccc34)cc12] | 74 |
| 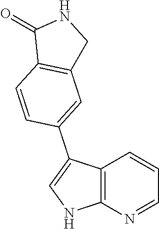 [SMILES: O=C1NCc2cc(-c3c[nH]c4ncccc34)ccc21] | 75 |
| 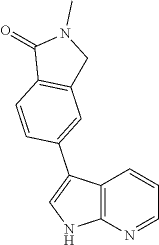 [SMILES: CN1Cc2cc(-c3c[nH]c4ncccc34)ccc2C1=O] | 76 |
| 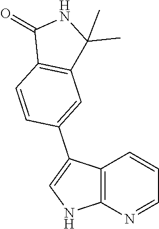 [SMILES: CC1(C)NC(=O)c2ccc(-c3c[nH]c4ncccc34)cc21] | 77 |
| 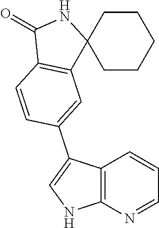 [SMILES: O=C1NC2(CCCCC2)c2cc(-c3c[nH]c4ncccc34)ccc21] | 78 |
| 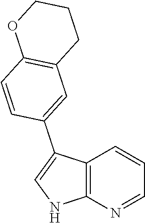 [SMILES: c1cnc2[nH]cc(-c3ccc4c(c3)CCCO4)c2c1] | 79 |
| 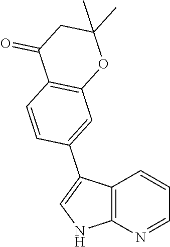 [SMILES: CC1(C)CC(=O)c2ccc(-c3c[nH]c4ncccc34)cc2O1] | 80 |
| 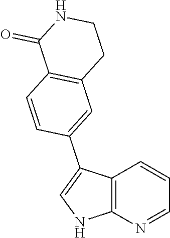 [SMILES: O=C1NCCc2cc(-c3c[nH]c4ncccc34)ccc21] | 81 |
| 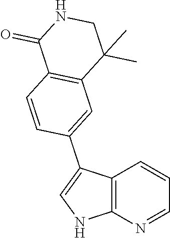 [SMILES: CC1(C)CNC(=O)c2ccc(-c3c[nH]c4ncccc34)cc21] | 82 |
| 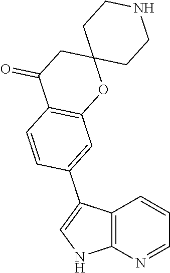 [SMILES: O=C1CC2(CCNCC2)Oc2cc(-c3c[nH]c4ncccc34)ccc21] | 83 |
| 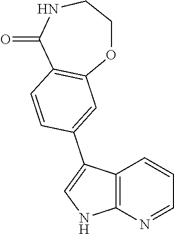 [SMILES: O=C1NCCOc2cc(-c3c[nH]c4ncccc34)ccc21] | 84 |
| 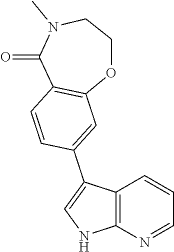 [SMILES: CN1CCOc2cc(-c3c[nH]c4ncccc34)ccc2C1=O] | 85 |
| 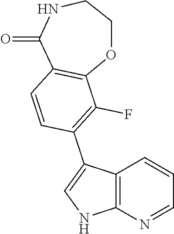 [SMILES: O=C1NCCOc2c1ccc(-c1c[nH]c3ncccc13)c2F] | 86 |
| 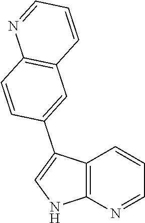 [SMILES: c1cnc2ccc(-c3c[nH]c4ncccc34)cc2c1] | 87 |
| 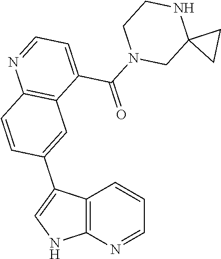 [SMILES: O=C(c1ccnc2ccc(-c3c[nH]c4ncccc34)cc12)N1CCNC2(CC2)C1] | 88 |
| 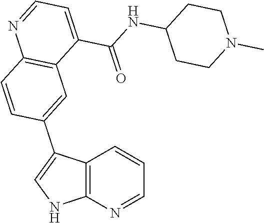 [SMILES: CN1CCC(NC(=O)c2ccnc3ccc(-c4c[nH]c5ncccc45)cc23)CC1] | 89 |
| 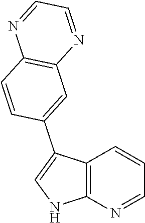 [SMILES: c1cnc2[nH]cc(-c3ccc4nccnc4c3)c2c1] | 90 |
| 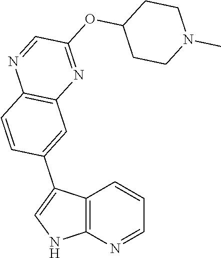 [SMILES: CN1CCC(Oc2cnc3ccc(-c4c[nH]c5ncccc45)cc3n2)CC1] | 91 |
| 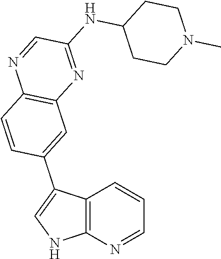 [SMILES: CN1CCC(Nc2cnc3ccc(-c4c[nH]c5ncccc45)cc3n2)CC1] | 92 |
| 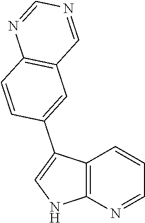 [SMILES: c1cnc2[nH]cc(-c3ccc4ncncc4c3)c2c1] | 93 |
| 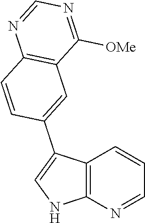 [SMILES: COc1ncnc2ccc(-c3c[nH]c4ncccc34)cc12] | 94 |
| 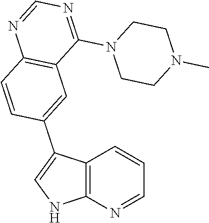 [SMILES: CN1CCN(c2ncnc3ccc(-c4c[nH]c5ncccc45)cc23)CC1] | 95 |
| 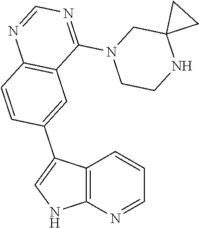 [SMILES: c1cnc2[nH]cc(-c3ccc4ncnc(N5CCNC6(CC6)C5)c4c3)c2c1] | 96 |
| 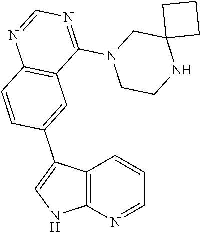 [SMILES: c1cnc2[nH]cc(-c3ccc4ncnc(N5CCNC6(CCC6)C5)c4c3)c2c1] | 97 |
| 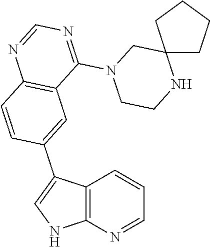 [SMILES: c1cnc2[nH]cc(-c3ccc4ncnc(N5CCNC6(CCCC6)C5)c4c3)c2c1] | 98 |
| 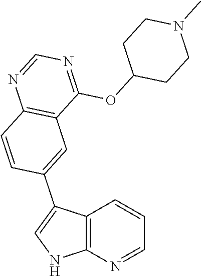 [SMILES: CN1CCC(Oc2ncnc3ccc(-c4c[nH]c5ncccc45)cc23)CC1] | 99 |
| 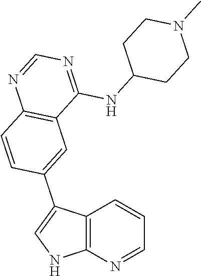 [SMILES: CN1CCC(Nc2ncnc3ccc(-c4c[nH]c5ncccc45)cc23)CC1] | 100 |
| 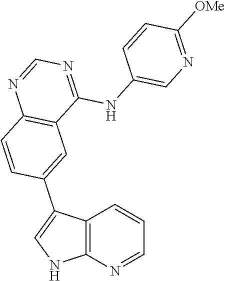 [SMILES: COc1ccc(Nc2ncnc3ccc(-c4c[nH]c5ncccc45)cc23)cn1] | 101 |
| 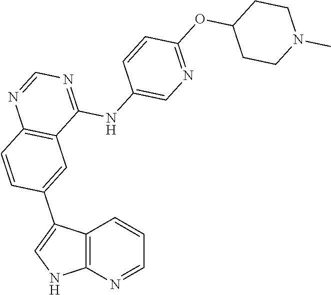 [SMILES: CN1CCC(Oc2ccc(Nc3ncnc4ccc(-c5c[nH]c6ncccc56)cc34)cn2)CC1] | 102 |
| 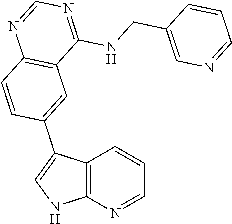 [SMILES: c1cncc(CNc2ncnc3ccc(-c4c[nH]c5ncccc45)cc23)c1] | 103 |
| 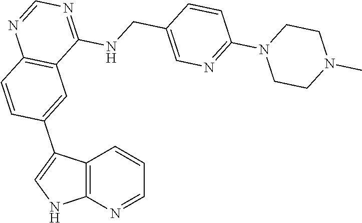 [SMILES: CN1CCN(c2ccc(CNc3ncnc4ccc(-c5c[nH]c6ncccc56)cc34)cn2)CC1] | 104 |
| 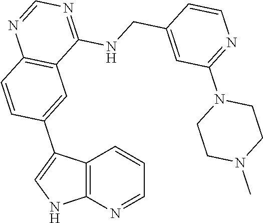 [SMILES: CN1CCN(c2cc(CNc3ncnc4ccc(-c5c[nH]c6ncccc56)cc34)ccn2)CC1] | 105 |
| 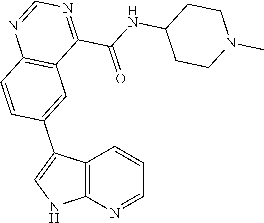 [SMILES: CN1CCC(NC(=O)c2ncnc3ccc(-c4c[nH]c5ncccc45)cc23)CC1] | 106 |
| 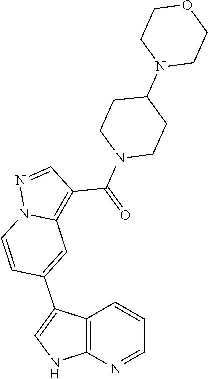 [SMILES: O=C(c1cnn2ccc(-c3c[nH]c4ncccc34)cc12)N1CCC(N2CCOCC2)CC1] | 107 |
| 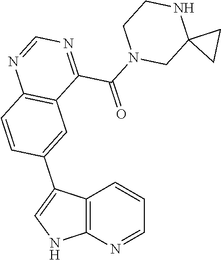 [SMILES: O=C(c1ncnc2ccc(-c3c[nH]c4ncccc34)cc12)N1CCNC2(CC2)C1] | 108 |
| 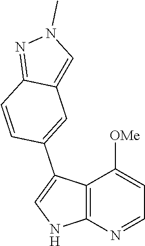 [SMILES: COc1ccnc2[nH]cc(-c3ccc4nn(C)cc4c3)c12] | 109 |
| 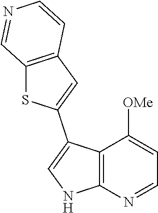 [SMILES: COc1ccnc2[nH]cc(-c3cc4ccncc4s3)c12] | 110 |
| 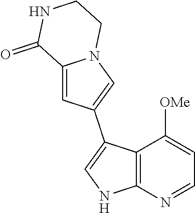 [SMILES: COc1ccnc2[nH]cc(-c3cc4n(c3)CCNC4=O)c12] | 111 |
| 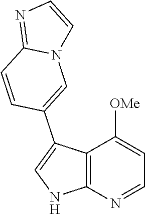 [SMILES: COc1ccnc2[nH]cc(-c3ccc4nccn4c3)c12] | 112 |
| 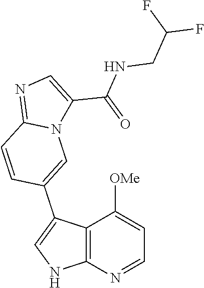 [SMILES: COc1ccnc2[nH]cc(-c3ccc4ncc(C(=O)NCC(F)F)n4c3)c12] | 113 |
| 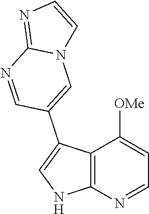 [SMILES: COc1ccnc2[nH]cc(-c3cnc4nccn4c3)c12] | 114 |
| 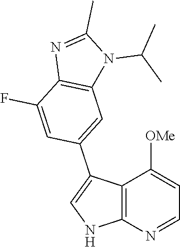 [SMILES: COc1ccnc2[nH]cc(-c3cc(F)c4nc(C)n(C(C)C)c4c3)c12] | 115 |
| 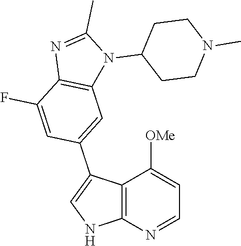 [SMILES: COc1ccnc2[nH]cc(-c3cc(F)c4nc(C)n(C5CCN(C)CC5)c4c3)c12] | 116 |
| 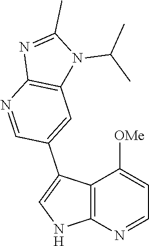 [SMILES: COc1ccnc2[nH]cc(-c3cnc4nc(C)n(C(C)C)c4c3)c12] | 117 |
| 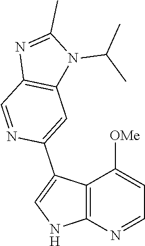 [SMILES: COc1ccnc2[nH]cc(-c3cc4c(cn3)nc(C)n4C(C)C)c12] | 118 |
| 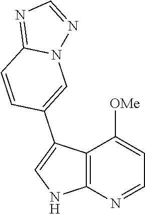 [SMILES: COc1ccnc2[nH]cc(-c3ccc4ncnn4c3)c12] | 119 |
| 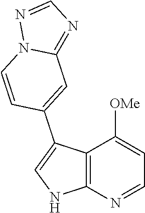 [SMILES: COc1ccnc2[nH]cc(-c3ccn4ncnc4c3)c12] | 120 |
| 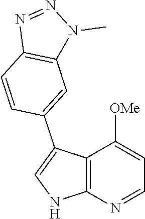 [SMILES: COc1ccnc2[nH]cc(-c3ccc4nnn(C)c4c3)c12] | 121 |
| 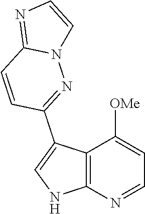 [SMILES: COc1ccnc2[nH]cc(-c3ccc4nccn4n3)c12] | 122 |
| 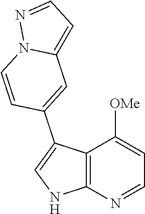 [SMILES: COc1ccnc2[nH]cc(-c3ccn4nccc4c3)c12] | 123 |
| 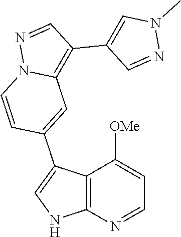 [SMILES: COc1ccnc2[nH]cc(-c3ccn4ncc(-c5cnn(C)c5)c4c3)c12] | 124 |
| 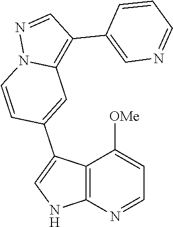 [SMILES: COc1ccnc2[nH]cc(-c3ccn4ncc(-c5cccnc5)c4c3)c12] | 125 |
| 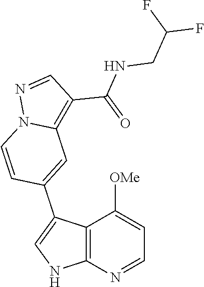 [SMILES: COc1ccnc2[nH]cc(-c3ccn4ncc(C(=O)NCC(F)F)c4c3)c12] | 126 |
| 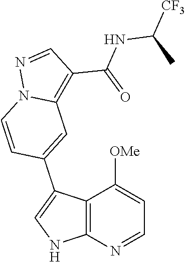 [SMILES: COc1ccnc2[nH]cc(-c3ccn4ncc(C(=O)N[C@H](C)C(F)(F)F)c4c3)c12] | 127 |
| 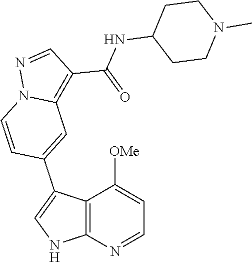 [SMILES: COc1ccnc2[nH]cc(-c3ccn4ncc(C(=O)NC5CCN(C)CC5)c4c3)c12] | 128 |
| 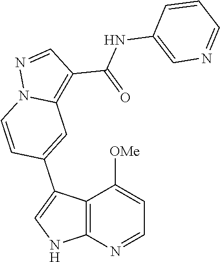 [SMILES: COc1ccnc2[nH]cc(-c3ccn4ncc(C(=O)Nc5cccnc5)c4c3)c12] | 129 |
| 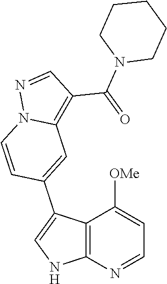 [SMILES: COc1ccnc2[nH]cc(-c3ccn4ncc(C(=O)N5CCCCC5)c4c3)c12] | 130 |
| 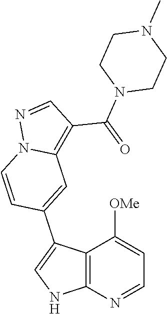 [SMILES: COc1ccnc2[nH]cc(-c3ccn4ncc(C(=O)N5CCN(C)CC5)c4c3)c12] | 131 |
| 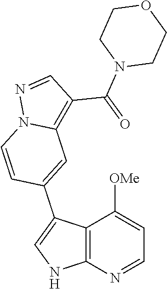 [SMILES: COc1ccnc2[nH]cc(-c3ccn4ncc(C(=O)N5CCOCC5)c4c3)c12] | 132 |
| 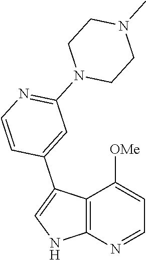 [SMILES: COc1ccnc2[nH]cc(-c3ccnc(N4CCN(C)CC4)c3)c12] | 133 |
| 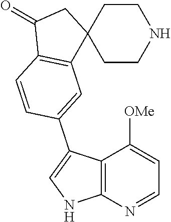 [SMILES: COc1ccnc2[nH]cc(-c3ccc4c(c3)C3(CCNCC3)CC4=O)c12] | 134 |
| 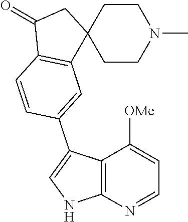 [SMILES: COc1ccnc2[nH]cc(-c3ccc4c(c3)C3(CCN(C)CC3)CC4=O)c12] | 135 |
| 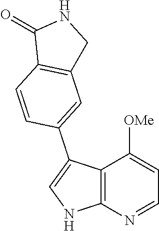 [SMILES: COc1ccnc2[nH]cc(-c3ccc4c(c3)CNC4=O)c12] | 136 |
| 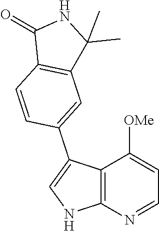 [SMILES: COc1ccnc2[nH]cc(-c3ccc4c(c3)C(C)(C)NC4=O)c12] | 137 |
| 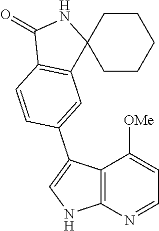 [SMILES: COc1ccnc2[nH]cc(-c3ccc4c(c3)C3(CCCCC3)NC4=O)c12] | 138 |
| 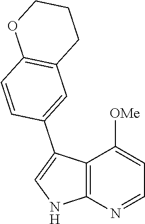 [SMILES: COc1ccnc2[nH]cc(-c3ccc4c(c3)CCCO4)c12] | 139 |
| 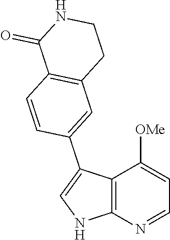 [SMILES: COc1ccnc2[nH]cc(-c3ccc4c(c3)CCNC4=O)c12] | 140 |
| 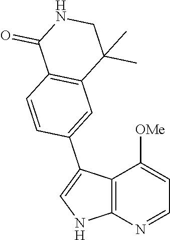 [SMILES: COc1ccnc2[nH]cc(-c3ccc4c(c3)C(C)(C)CNC4=O)c12] | 141 |
| 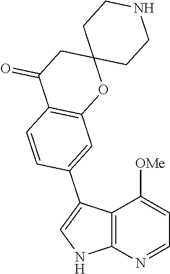 [SMILES: COc1ccnc2[nH]cc(-c3ccc4c(c3)OC3(CCNCC3)CC4=O)c12] | 142 |
| 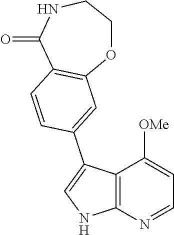 [SMILES: COc1ccnc2[nH]cc(-c3ccc4c(c3)OCCNC4=O)c12] | 143 |
| 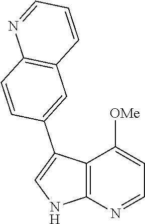 [SMILES: COc1ccnc2[nH]cc(-c3ccc4ncccc4c3)c12] | 144 |
| 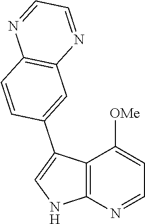 [SMILES: COc1ccnc2[nH]cc(-c3ccc4nccnc4c3)c12] | 145 |
| 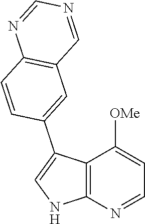 [SMILES: COc1ccnc2[nH]cc(-c3ccc4ncncc4c3)c12] | 146 |
| 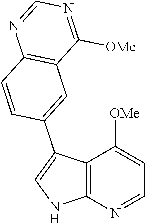 [SMILES: COc1ncnc2ccc(-c3c[nH]c4nccc(OC)c34)cc12] | 147 |
| 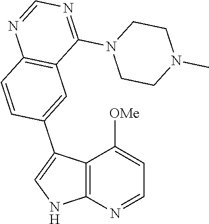 [SMILES: COc1ccnc2[nH]cc(-c3ccc4ncnc(N5CCN(C)CC5)c4c3)c12] | 148 |
| 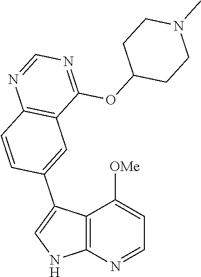 [SMILES: COc1ccnc2[nH]cc(-c3ccc4ncnc(OC5CCN(C)CC5)c4c3)c12] | 149 |
| 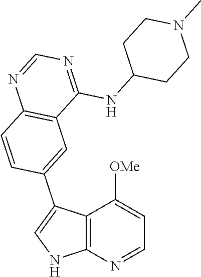 [SMILES: COc1ccnc2[nH]cc(-c3ccc4ncnc(NC5CCN(C)CC5)c4c3)c12] | 150 |
| 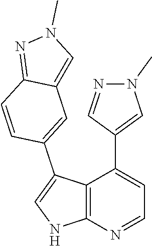 [SMILES: Cn1cc(-c2ccnc3[nH]cc(-c4ccc5nn(C)cc5c4)c23)cn1] | 151 |
| 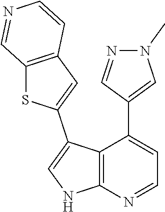 [SMILES: Cn1cc(-c2ccnc3[nH]cc(-c4cc5ccncc5s4)c23)cn1] | 152 |
| 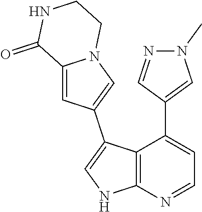 [SMILES: Cn1cc(-c2ccnc3[nH]cc(-c4cc5n(c4)CCNC5=O)c23)cn1] | 153 |
| 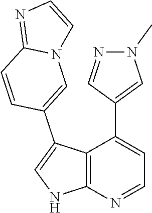 [SMILES: Cn1cc(-c2ccnc3[nH]cc(-c4ccc5nccn5c4)c23)cn1] | 154 |
| 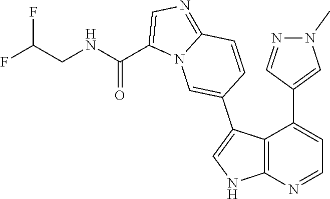 [SMILES: Cn1cc(-c2ccnc3[nH]cc(-c4ccc5ncc(C(=O)NCC(F)F)n5c4)c23)cn1] | 155 |
| 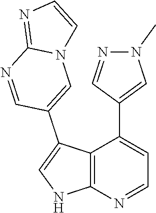 [SMILES: Cn1cc(-c2ccnc3[nH]cc(-c4cnc5nccn5c4)c23)cn1] | 156 |
| 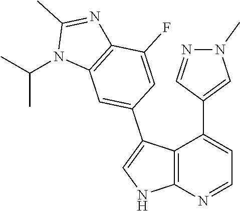 [SMILES: Cc1nc2c(F)cc(-c3c[nH]c4nccc(-c5cnn(C)c5)c34)cc2n1C(C)C] | 157 |
| 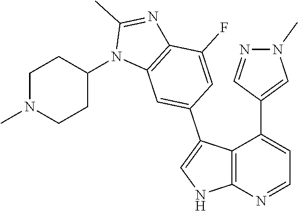 | 158 |
| 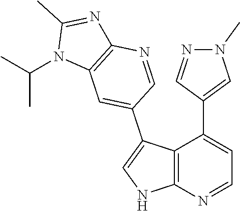 [SMILES: Cc1nc2ncc(-c3c[nH]c4nccc(-c5cnn(C)c5)c34)cc2n1C(C)C] | 159 |
| 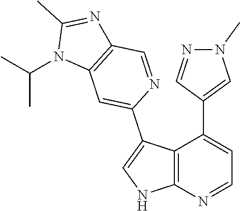 [SMILES: Cc1nc2cnc(-c3c[nH]c4nccc(-c5cnn(C)c5)c34)cc2n1C(C)C] | 160 |
| 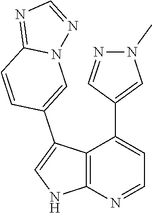 [SMILES: Cn1cc(-c2ccnc3[nH]cc(-c4ccc5ncnn5c4)c23)cn1] | 161 |
| 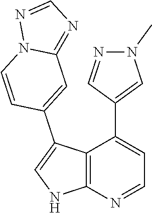 [SMILES: Cn1cc(-c2ccnc3[nH]cc(-c4ccn5ncnc5c4)c23)cn1] | 162 |
| 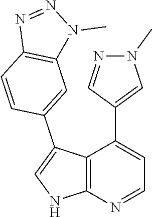 [SMILES: Cn1cc(-c2ccnc3[nH]cc(-c4ccc5nnn(C)c5c4)c23)cn1] | 163 |
| 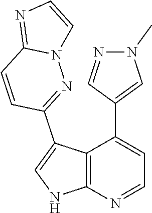 [SMILES: Cn1cc(-c2ccnc3[nH]cc(-c4ccc5nccn5n4)c23)cn1] | 164 |
| 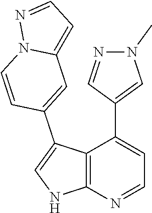 [SMILES: Cn1cc(-c2ccnc3[nH]cc(-c4ccn5nccc5c4)c23)cn1] | 165 |
| 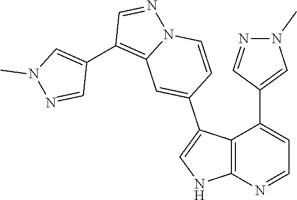 | 166 |
| 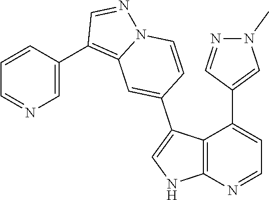 [SMILES: Cn1cc(-c2ccnc3[nH]cc(-c4ccn5ncc(-c6cccnc6)c5c4)c23)cn1] | 167 |
| 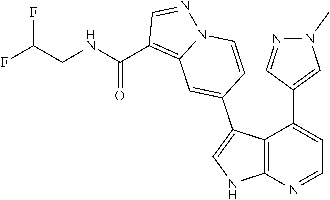 [SMILES: Cn1cc(-c2ccnc3[nH]cc(-c4ccn5ncc(C(=O)NCC(F)F)c5c4)c23)cn1] | 168 |
| 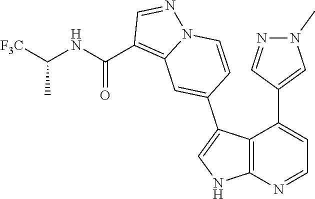 [SMILES: C[C@@H](NC(=O)c1cnn2ccc(-c3c[nH]c4nccc(-c5cnn(C)c5)c34)cc12)C(F)(F)F] | 169 |
| 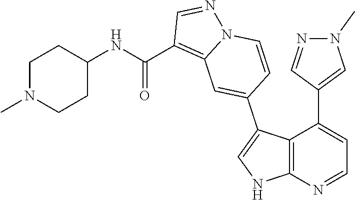 | 170 |
| 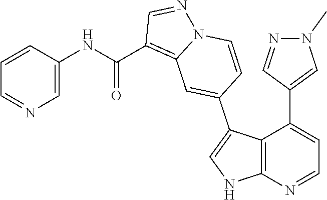 [SMILES: Cn1cc(-c2ccnc3[nH]cc(-c4ccn5ncc(C(=O)Nc6cccnc6)c5c4)c23)cn1] | 171 |
| 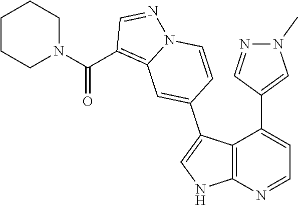 [SMILES: Cn1cc(-c2ccnc3[nH]cc(-c4ccn5ncc(C(=O)N6CCCCC6)c5c4)c23)cn1] | 172 |
| 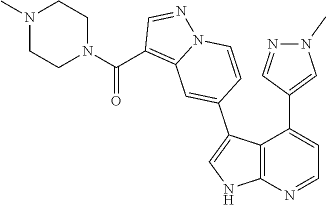 | 173 |
| 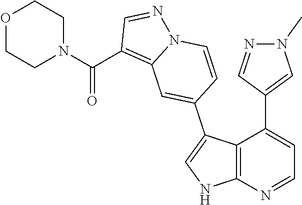 [SMILES: Cn1cc(-c2ccnc3[nH]cc(-c4ccn5ncc(C(=O)N6CCOCC6)c5c4)c23)cn1] | 174 |
| 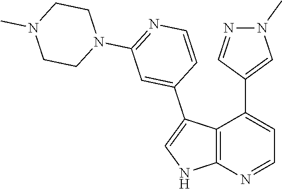 | 175 |
| 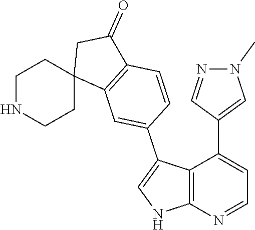 [SMILES: Cn1cc(-c2ccnc3[nH]cc(-c4ccc5c(c4)C4(CCNCC4)CC5=O)c23)cn1] | 176 |
| 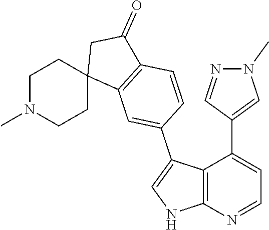 | 177 |
| 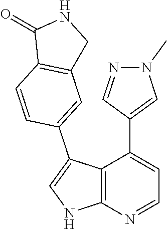 [SMILES: Cn1cc(-c2ccnc3[nH]cc(-c4ccc5c(c4)CNC5=O)c23)cn1] | 178 |
| 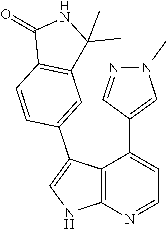 [SMILES: Cn1cc(-c2ccnc3[nH]cc(-c4ccc5c(c4)C(C)(C)NC5=O)c23)cn1] | 179 |
| 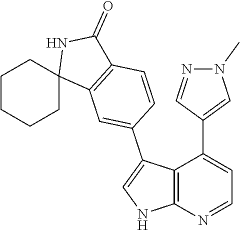 [SMILES: Cn1cc(-c2ccnc3[nH]cc(-c4ccc5c(c4)C4(CCCCC4)NC5=O)c23)cn1] | 180 |
| 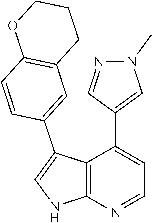 [SMILES: Cn1cc(-c2ccnc3[nH]cc(-c4ccc5c(c4)CCCO5)c23)cn1] | 181 |
| 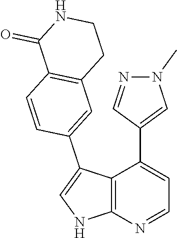 [SMILES: Cn1cc(-c2ccnc3[nH]cc(-c4ccc5c(c4)CCNC5=O)c23)cn1] | 182 |
| 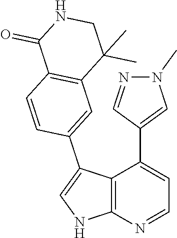 [SMILES: Cn1cc(-c2ccnc3[nH]cc(-c4ccc5c(c4)C(C)(C)CNC5=O)c23)cn1] | 183 |
| 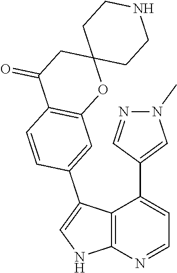 [SMILES: Cn1cc(-c2ccnc3[nH]cc(-c4ccc5c(c4)OC4(CCNCC4)CC5=O)c23)cn1] | 184 |
| 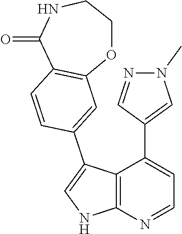 [SMILES: Cn1cc(-c2ccnc3[nH]cc(-c4ccc5c(c4)OCCNC5=O)c23)cn1] | 185 |
| 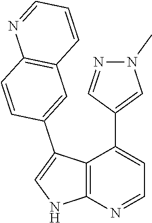 [SMILES: Cn1cc(-c2ccnc3[nH]cc(-c4ccc5ncccc5c4)c23)cn1] | 186 |
| 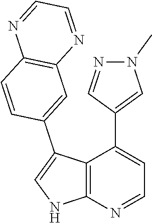 [SMILES: Cn1cc(-c2ccnc3[nH]cc(-c4ccc5nccnc5c4)c23)cn1] | 187 |
| 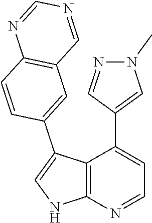 [SMILES: Cn1cc(-c2ccnc3[nH]cc(-c4ccc5ncncc5c4)c23)cn1] | 188 |
| 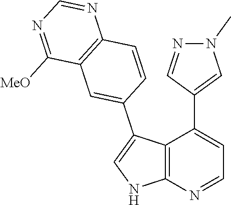 [SMILES: COc1ncnc2ccc(-c3c[nH]c4nccc(-c5cnn(C)c5)c34)cc12] | 189 |
| 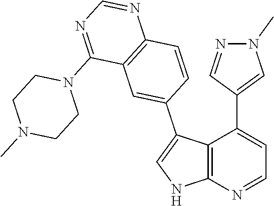 | 190 |
| 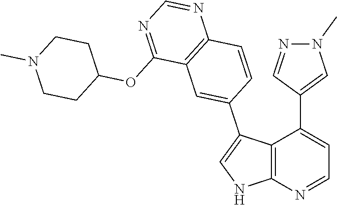 | 191 |
| 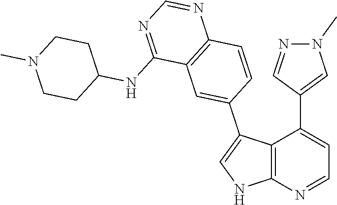 | 192 |
| 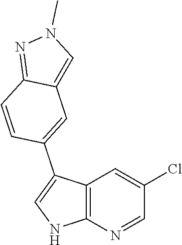 [SMILES: Cn1cc2cc(-c3c[nH]c4ncc(Cl)cc34)ccc2n1] | 193 |
| 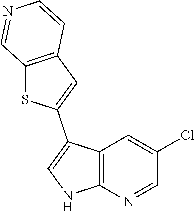 [SMILES: Clc1cnc2[nH]cc(-c3cc4ccncc4s3)c2c1] | 194 |
| 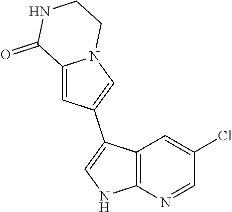 [SMILES: O=C1NCCn2cc(-c3c[nH]c4ncc(Cl)cc34)cc21] | 195 |
| 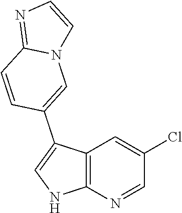 [SMILES: Clc1cnc2[nH]cc(-c3ccc4nccn4c3)c2c1] | 196 |
| 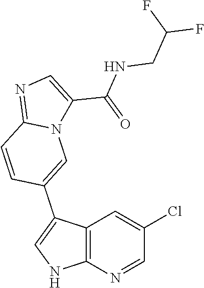 [SMILES: O=C(NCC(F)F)c1cnc2ccc(-c3c[nH]c4ncc(Cl)cc34)cn12] | 197 |
| 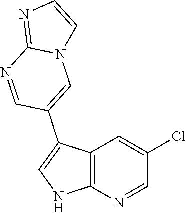 [SMILES: Clc1cnc2[nH]cc(-c3cnc4nccn4c3)c2c1] | 198 |
| 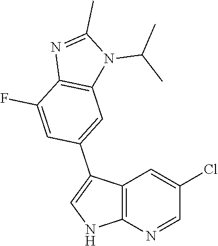 [SMILES: Cc1nc2c(F)cc(-c3c[nH]c4ncc(Cl)cc34)cc2n1C(C)C] | 199 |
| 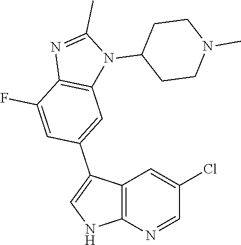 [SMILES: Cc1nc2c(F)cc(-c3c[nH]c4ncc(Cl)cc34)cc2n1C1CCN(C)CC1] | 200 |
| 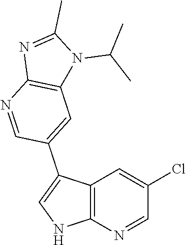 [SMILES: Cc1nc2ncc(-c3c[nH]c4ncc(Cl)cc34)cc2n1C(C)C] | 201 |
| 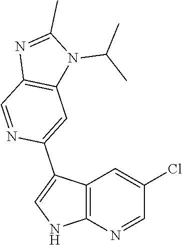 [SMILES: Cc1nc2cnc(-c3c[nH]c4ncc(Cl)cc34)cc2n1C(C)C] | 202 |
| 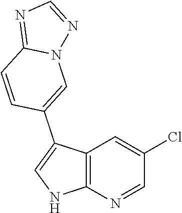 [SMILES: Clc1cnc2[nH]cc(-c3ccc4ncnn4c3)c2c1] | 203 |
| 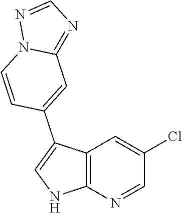 [SMILES: Clc1cnc2[nH]cc(-c3ccn4ncnc4c3)c2c1] | 204 |
| 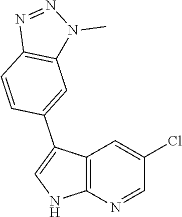 [SMILES: Cn1nnc2ccc(-c3c[nH]c4ncc(Cl)cc34)cc21] | 205 |
| 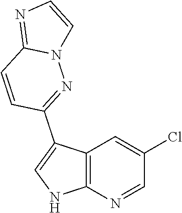 [SMILES: Clc1cnc2[nH]cc(-c3ccc4nccn4n3)c2c1] | 206 |
| 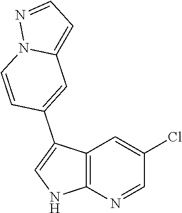 [SMILES: Clc1cnc2[nH]cc(-c3ccn4nccc4c3)c2c1] | 207 |
| 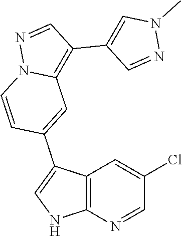 [SMILES: Cn1cc(-c2cnn3ccc(-c4c[nH]c5ncc(Cl)cc45)cc23)cn1] | 208 |
| 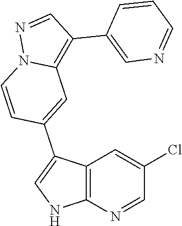 [SMILES: Clc1cnc2[nH]cc(-c3ccn4ncc(-c5cccnc5)c4c3)c2c1] | 209 |
| 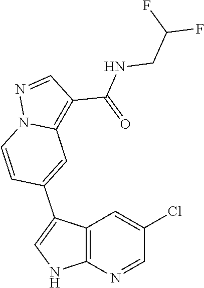 [SMILES: O=C(NCC(F)F)c1cnn2ccc(-c3c[nH]c4ncc(Cl)cc34)cc12] | 210 |
| 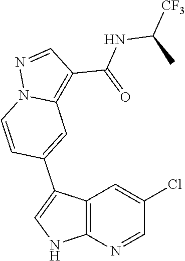 [SMILES: C[C@@H](NC(=O)c1cnn2ccc(-c3c[nH]c4ncc(Cl)cc34)cc12)C(F)(F)F] | 211 |
| 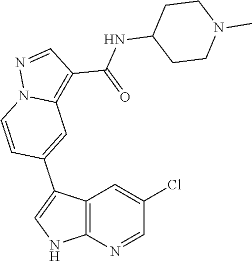 [SMILES: CN1CCC(NC(=O)c2cnn3ccc(-c4c[nH]c5ncc(Cl)cc45)cc23)CC1] | 212 |
| 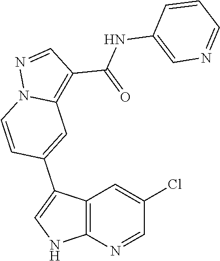 [SMILES: O=C(Nc1cccnc1)c1cnn2ccc(-c3c[nH]c4ncc(Cl)cc34)cc12] | 213 |
| 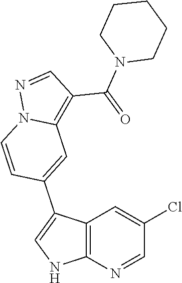 [SMILES: O=C(c1cnn2ccc(-c3c[nH]c4ncc(Cl)cc34)cc12)N1CCCCC1] | 214 |
| 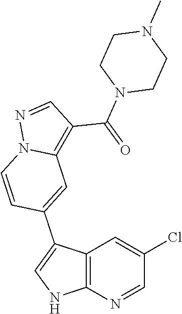 [SMILES: CN1CCN(C(=O)c2cnn3ccc(-c4c[nH]c5ncc(Cl)cc45)cc23)CC1] | 215 |
| 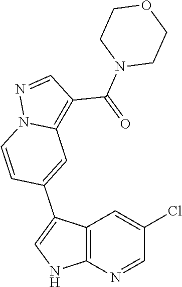 [SMILES: O=C(c1cnn2ccc(-c3c[nH]c4ncc(Cl)cc34)cc12)N1CCOCC1] | 216 |
| 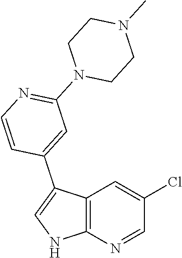 [SMILES: CN1CCN(c2cc(-c3c[nH]c4ncc(Cl)cc34)ccn2)CC1] | 217 |
| 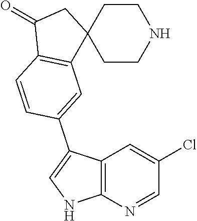 [SMILES: O=C1CC2(CCNCC2)c2cc(-c3c[nH]c4ncc(Cl)cc34)ccc21] | 218 |
| 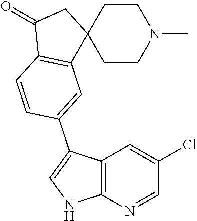 [SMILES: CN1CCC2(CC1)CC(=O)c1ccc(-c3c[nH]c4ncc(Cl)cc34)cc12] | 219 |
| 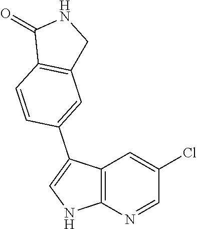 [SMILES: O=C1NCc2cc(-c3c[nH]c4ncc(Cl)cc34)ccc21] | 220 |
| 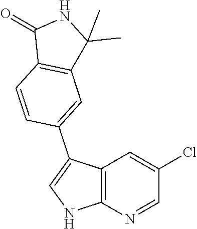 [SMILES: CC1(C)NC(=O)c2ccc(-c3c[nH]c4ncc(Cl)cc34)cc21] | 221 |
| 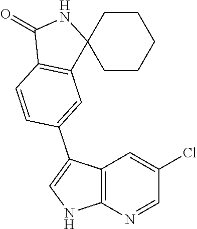 [SMILES: O=C1NC2(CCCCC2)c2cc(-c3c[nH]c4ncc(Cl)cc34)ccc21] | 222 |
| 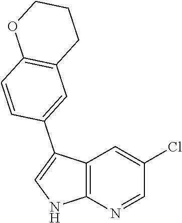 [SMILES: Clc1cnc2[nH]cc(-c3ccc4c(c3)CCCO4)c2c1] | 223 |
| 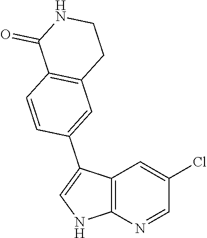 [SMILES: O=C1NCCc2cc(-c3c[nH]c4ncc(Cl)cc34)ccc21] | 224 |
| 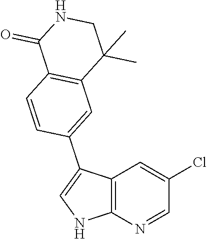 [SMILES: CC1(C)CNC(=O)c2ccc(-c3c[nH]c4ncc(Cl)cc34)cc21] | 225 |
| 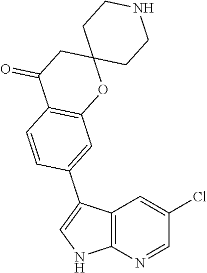 [SMILES: O=C1CC2(CCNCC2)Oc2cc(-c3c[nH]c4ncc(Cl)cc34)ccc21] | 226 |
| 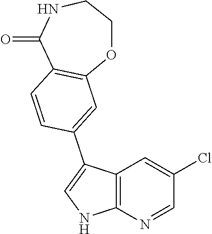 [SMILES: O=C1NCCOc2cc(-c3c[nH]c4ncc(Cl)cc34)ccc21] | 227 |
| 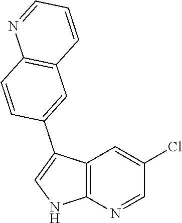 [SMILES: Clc1cnc2[nH]cc(-c3ccc4ncccc4c3)c2c1] | 228 |
| 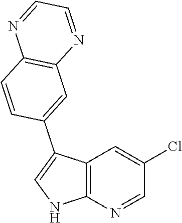 [SMILES: Clc1cnc2[nH]cc(-c3ccc4nccnc4c3)c2c1] | 229 |
| 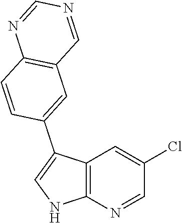 [SMILES: Clc1cnc2[nH]cc(-c3ccc4ncncc4c3)c2c1] | 230 |
| 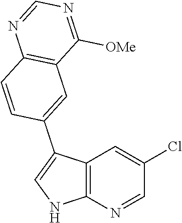 [SMILES: COc1ncnc2ccc(-c3c[nH]c4ncc(Cl)cc34)cc12] | 231 |
| 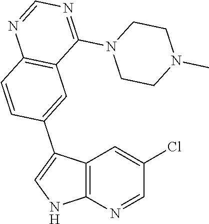 [SMILES: CN1CCN(c2ncnc3ccc(-c4c[nH]c5ncc(Cl)cc45)cc23)CC1] | 232 |
| 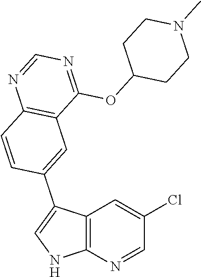 [SMILES: CN1CCC(Oc2ncnc3ccc(-c4c[nH]c5ncc(Cl)cc45)cc23)CC1] | 233 |
| 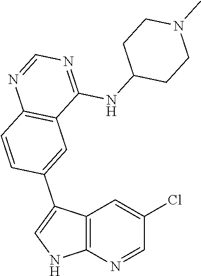 [SMILES: CN1CCC(Nc2ncnc3ccc(-c4c[nH]c5ncc(Cl)cc45)cc23)CC1] | 234 |
| 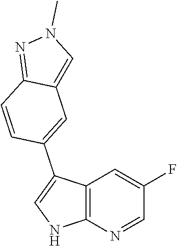 [SMILES: Cn1cc2cc(-c3c[nH]c4ncc(F)cc34)ccc2n1] | 235 |
| 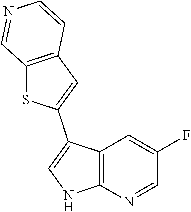 [SMILES: Fc1cnc2[nH]cc(-c3cc4ccncc4s3)c2c1] | 236 |
| 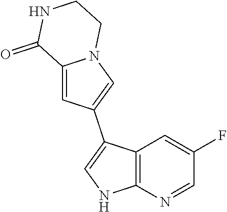 [SMILES: O=C1NCCn2cc(-c3c[nH]c4ncc(F)cc34)cc21] | 237 |
| 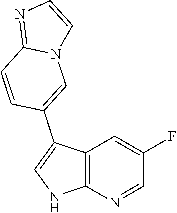 [SMILES: Fc1cnc2[nH]cc(-c3ccc4nccn4c3)c2c1] | 238 |
| 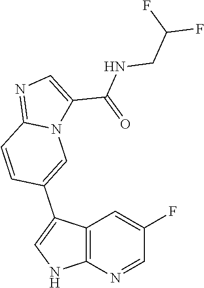 [SMILES: O=C(NCC(F)F)c1cnc2ccc(-c3c[nH]c4ncc(F)cc34)cn12] | 239 |
| 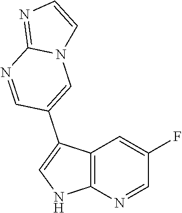 [SMILES: Fc1cnc2[nH]cc(-c3cnc4nccn4c3)c2c1] | 240 |
| 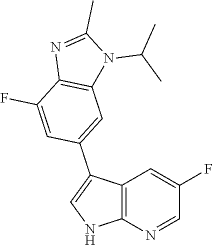 [SMILES: Cc1nc2c(F)cc(-c3c[nH]c4ncc(F)cc34)cc2n1C(C)C] | 241 |
| 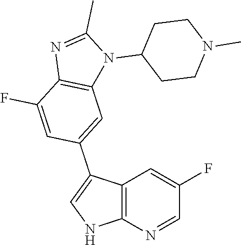 [SMILES: Cc1nc2c(F)cc(-c3c[nH]c4ncc(F)cc34)cc2n1C1CCN(C)CC1] | 242 |
| 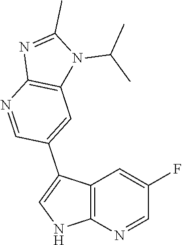 [SMILES: Cc1nc2ncc(-c3c[nH]c4ncc(F)cc34)cc2n1C(C)C] | 243 |
| 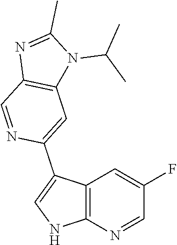 [SMILES: Cc1nc2cnc(-c3c[nH]c4ncc(F)cc34)cc2n1C(C)C] | 244 |
| 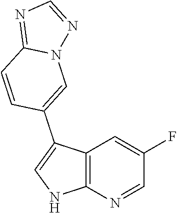 [SMILES: Fc1cnc2[nH]cc(-c3ccc4ncnn4c3)c2c1] | 245 |
| 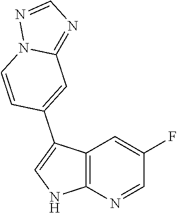 [SMILES: Fc1cnc2[nH]cc(-c3ccn4ncnc4c3)c2c1] | 246 |
| 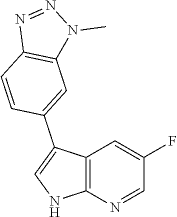 [SMILES: Cn1nnc2ccc(-c3c[nH]c4ncc(F)cc34)cc21] | 247 |
| 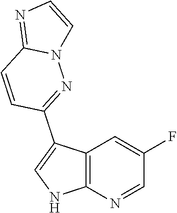 [SMILES: Fc1cnc2[nH]cc(-c3ccc4nccn4n3)c2c1] | 248 |
| 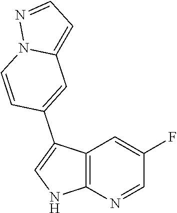 [SMILES: Fc1cnc2[nH]cc(-c3ccn4nccc4c3)c2c1] | 249 |
| 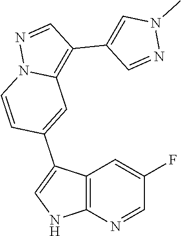 [SMILES: Cn1cc(-c2cnn3ccc(-c4c[nH]c5ncc(F)cc45)cc23)cn1] | 250 |
| 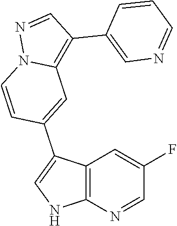 [SMILES: Fc1cnc2[nH]cc(-c3ccn4ncc(-c5cccnc5)c4c3)c2c1] | 251 |
| 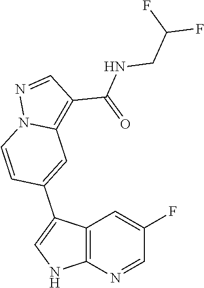 [SMILES: O=C(NCC(F)F)c1cnn2ccc(-c3c[nH]c4ncc(F)cc34)cc12] | 252 |
| 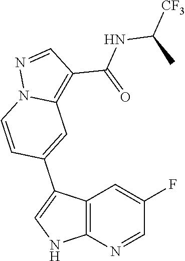 [SMILES: C[C@@H](NC(=O)c1cnn2ccc(-c3c[nH]c4ncc(F)cc34)cc12)C(F)(F)F] | 253 |
| 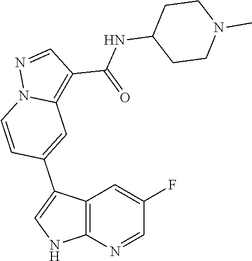 [SMILES: CN1CCC(NC(=O)c2cnn3ccc(-c4c[nH]c5ncc(F)cc45)cc23)CC1] | 254 |
| 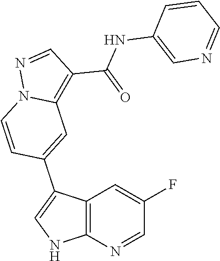 [SMILES: O=C(Nc1cccnc1)c1cnn2ccc(-c3c[nH]c4ncc(F)cc34)cc12] | 255 |
| 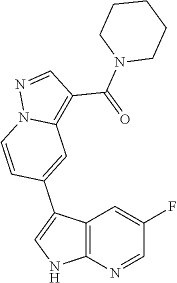 [SMILES: O=C(c1cnn2ccc(-c3c[nH]c4ncc(F)cc34)cc12)N1CCCCC1] | 256 |
| 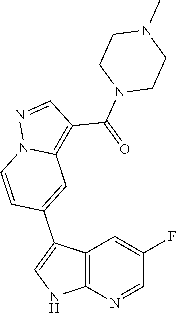 [SMILES: CN1CCN(C(=O)c2cnn3ccc(-c4c[nH]c5ncc(F)cc45)cc23)CC1] | 257 |
| 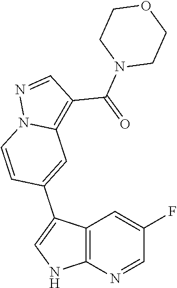 [SMILES: O=C(c1cnn2ccc(-c3c[nH]c4ncc(F)cc34)cc12)N1CCOCC1] | 258 |
| 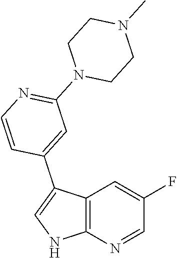 [SMILES: CN1CCN(c2cc(-c3c[nH]c4ncc(F)cc34)ccn2)CC1] | 259 |
| 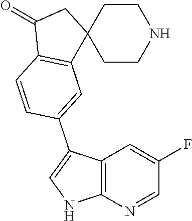 [SMILES: O=C1CC2(CCNCC2)c2cc(-c3c[nH]c4ncc(F)cc34)ccc21] | 260 |
| 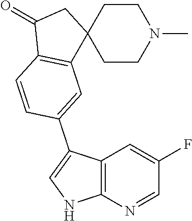 [SMILES: CN1CCC2(CC1)CC(=O)c1ccc(-c3c[nH]c4ncc(F)cc34)cc12] | 261 |
| 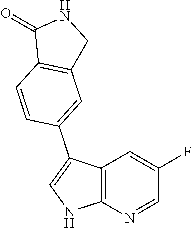 [SMILES: O=C1NCc2cc(-c3c[nH]c4ncc(F)cc34)ccc21] | 262 |
| 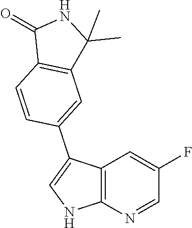 [SMILES: CC1(C)NC(=O)c2ccc(-c3c[nH]c4ncc(F)cc34)cc21] | 263 |
| 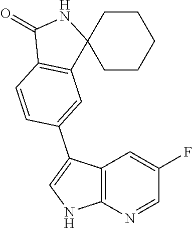 [SMILES: O=C1NC2(CCCCC2)c2cc(-c3c[nH]c4ncc(F)cc34)ccc21] | 264 |
| 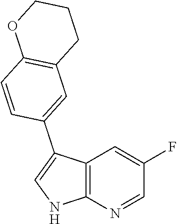 [SMILES: Fc1cnc2[nH]cc(-c3ccc4c(c3)CCCO4)c2c1] | 265 |
| 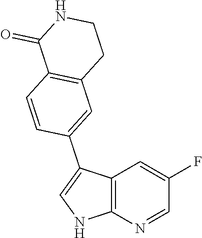 [SMILES: O=C1NCCc2cc(-c3c[nH]c4ncc(F)cc34)ccc21] | 266 |
| 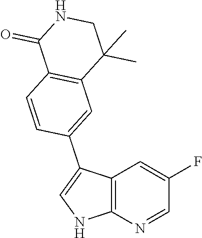 [SMILES: CC1(C)CNC(=O)c2ccc(-c3c[nH]c4ncc(F)cc34)cc21] | 267 |
| 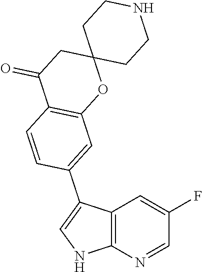 [SMILES: O=C1CC2(CCNCC2)Oc2cc(-c3c[nH]c4ncc(F)cc34)ccc21] | 268 |
| 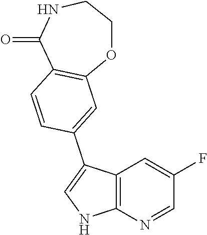 [SMILES: O=C1NCCOc2cc(-c3c[nH]c4ncc(F)cc34)ccc21] | 269 |
| 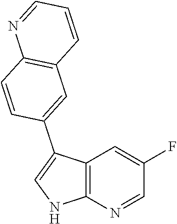 [SMILES: Fc1cnc2[nH]cc(-c3ccc4ncccc4c3)c2c1] | 270 |
| 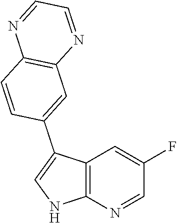 [SMILES: Fc1cnc2[nH]cc(-c3ccc4nccnc4c3)c2c1] | 271 |
| 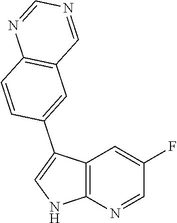 [SMILES: Fc1cnc2[nH]cc(-c3ccc4ncncc4c3)c2c1] | 272 |
| 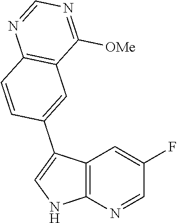 [SMILES: COc1ncnc2ccc(-c3c[nH]c4ncc(F)cc34)cc12] | 273 |
| 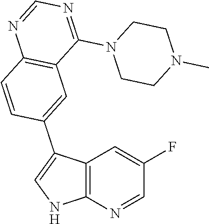 [SMILES: CN1CCN(c2ncnc3ccc(-c4c[nH]c5ncc(F)cc45)cc23)CC1] | 274 |
| 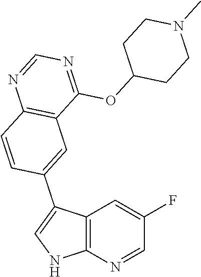 [SMILES: CN1CCC(Oc2ncnc3ccc(-c4c[nH]c5ncc(F)cc45)cc23)CC1] | 275 |
| 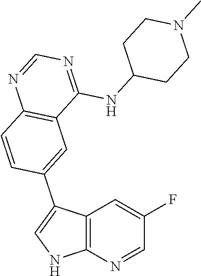 [SMILES: CN1CCC(Nc2ncnc3ccc(-c4c[nH]c5ncc(F)cc45)cc23)CC1] | 276 |
| 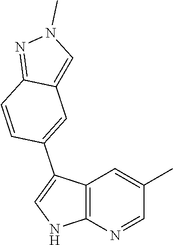 [SMILES: Cc1cnc2[nH]cc(-c3ccc4nn(C)cc4c3)c2c1] | 277 |
| 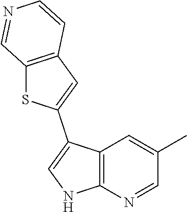 [SMILES: Cc1cnc2[nH]cc(-c3cc4ccncc4s3)c2c1] | 278 |
| 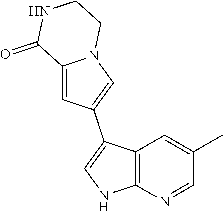 [SMILES: Cc1cnc2[nH]cc(-c3cc4n(c3)CCNC4=O)c2c1] | 279 |
| 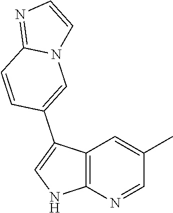 [SMILES: Cc1cnc2[nH]cc(-c3ccc4nccn4c3)c2c1] | 280 |
| 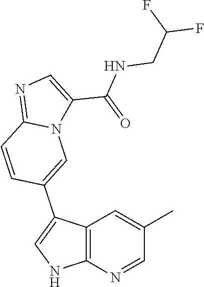 [SMILES: Cc1cnc2[nH]cc(-c3ccc4ncc(C(=O)NCC(F)F)n4c3)c2c1] | 281 |
| 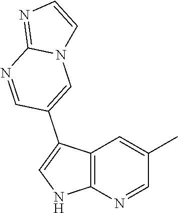 [SMILES: Cc1cnc2[nH]cc(-c3cnc4nccn4c3)c2c1] | 282 |
| 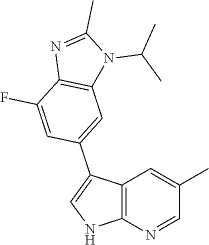 [SMILES: Cc1cnc2[nH]cc(-c3cc(F)c4nc(C)n(C(C)C)c4c3)c2c1] | 283 |
| 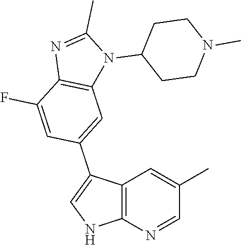 [SMILES: Cc1cnc2[nH]cc(-c3cc(F)c4nc(C)n(C5CCN(C)CC5)c4c3)c2c1] | 284 |
| 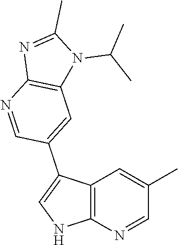 [SMILES: Cc1cnc2[nH]cc(-c3cnc4nc(C)n(C(C)C)c4c3)c2c1] | 285 |
| 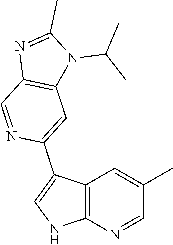 [SMILES: Cc1cnc2[nH]cc(-c3cc4c(cn3)nc(C)n4C(C)C)c2c1] | 286 |
| 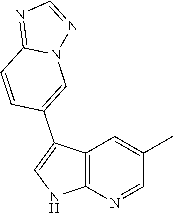 [SMILES: Cc1cnc2[nH]cc(-c3ccc4ncnn4c3)c2c1] | 287 |
| 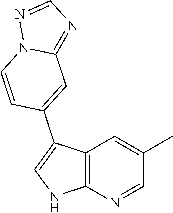 [SMILES: Cc1cnc2[nH]cc(-c3ccn4ncnc4c3)c2c1] | 288 |
| 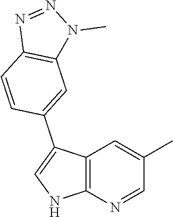 [SMILES: Cc1cnc2[nH]cc(-c3ccc4nnn(C)c4c3)c2c1] | 289 |
| 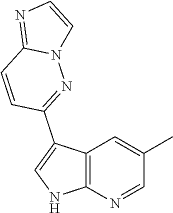 [SMILES: Cc1cnc2[nH]cc(-c3ccc4nccn4n3)c2c1] | 290 |
| 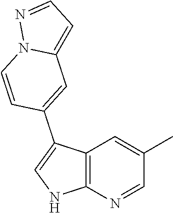 [SMILES: Cc1cnc2[nH]cc(-c3ccn4nccc4c3)c2c1] | 291 |
| 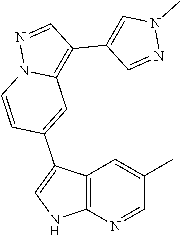 [SMILES: Cc1cnc2[nH]cc(-c3ccn4ncc(-c5cnn(C)c5)c4c3)c2c1] | 292 |
| 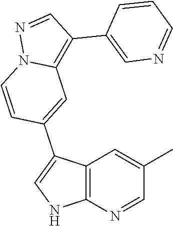 [SMILES: Cc1cnc2[nH]cc(-c3ccn4ncc(-c5cccnc5)c4c3)c2c1] | 293 |
| 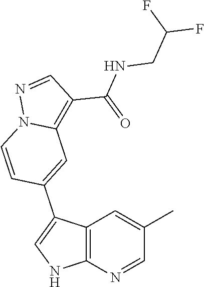 [SMILES: Cc1cnc2[nH]cc(-c3ccn4ncc(C(=O)NCC(F)F)c4c3)c2c1] | 294 |
| 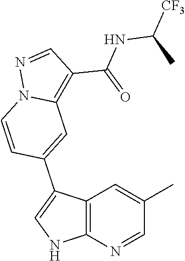 [SMILES: Cc1cnc2[nH]cc(-c3ccn4ncc(C(=O)N[C@H](C)C(F)(F)F)c4c3)c2c1] | 295 |
| 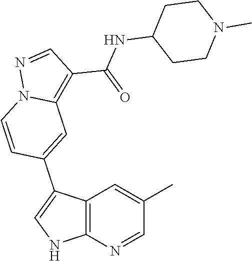 [SMILES: Cc1cnc2[nH]cc(-c3ccn4ncc(C(=O)NC5CCN(C)CC5)c4c3)c2c1] | 296 |
| 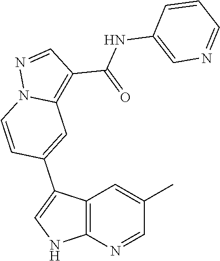 [SMILES: Cc1cnc2[nH]cc(-c3ccn4ncc(C(=O)Nc5cccnc5)c4c3)c2c1] | 297 |
| 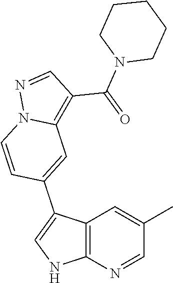 [SMILES: Cc1cnc2[nH]cc(-c3ccn4ncc(C(=O)N5CCCCC5)c4c3)c2c1] | 298 |
| 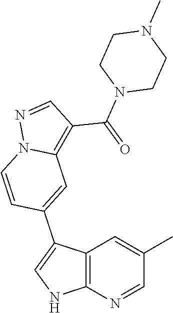 [SMILES: Cc1cnc2[nH]cc(-c3ccn4ncc(C(=O)N5CCN(C)CC5)c4c3)c2c1] | 299 |
| 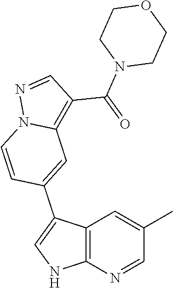 [SMILES: Cc1cnc2[nH]cc(-c3ccn4ncc(C(=O)N5CCOCC5)c4c3)c2c1] | 300 |
| 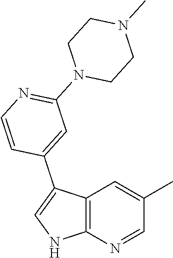 [SMILES: Cc1cnc2[nH]cc(-c3ccnc(N4CCN(C)CC4)c3)c2c1] | 301 |
| 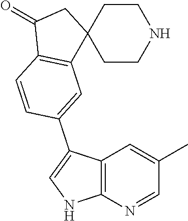 [SMILES: Cc1cnc2[nH]cc(-c3ccc4c(c3)C3(CCNCC3)CC4=O)c2c1] | 302 |
| 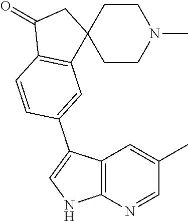 [SMILES: Cc1cnc2[nH]cc(-c3ccc4c(c3)C3(CCN(C)CC3)CC4=O)c2c1] | 303 |
| 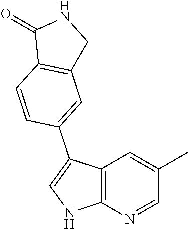 [SMILES: Cc1cnc2[nH]cc(-c3ccc4c(c3)CNC4=O)c2c1] | 304 |
| 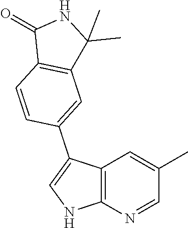 [SMILES: Cc1cnc2[nH]cc(-c3ccc4c(c3)C(C)(C)NC4=O)c2c1] | 305 |
| 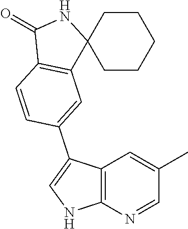 [SMILES: Cc1cnc2[nH]cc(-c3ccc4c(c3)C3(CCCCC3)NC4=O)c2c1] | 306 |
| 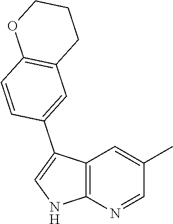 [SMILES: Cc1cnc2[nH]cc(-c3ccc4c(c3)CCCO4)c2c1] | 307 |
| 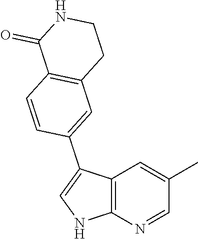 [SMILES: Cc1cnc2[nH]cc(-c3ccc4c(c3)CCNC4=O)c2c1] | 308 |
| 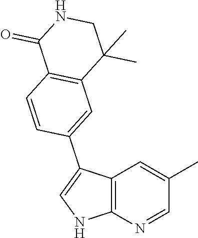 [SMILES: Cc1cnc2[nH]cc(-c3ccc4c(c3)C(C)(C)CNC4=O)c2c1] | 309 |
| 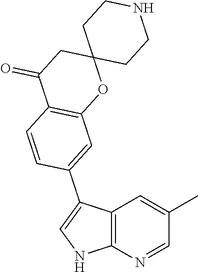 [SMILES: Cc1cnc2[nH]cc(-c3ccc4c(c3)OC3(CCNCC3)CC4=O)c2c1] | 310 |
| 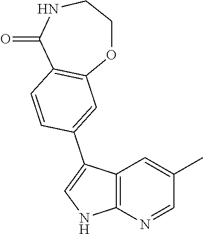 [SMILES: Cc1cnc2[nH]cc(-c3ccc4c(c3)OCCNC4=O)c2c1] | 311 |
| 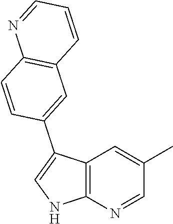 [SMILES: Cc1cnc2[nH]cc(-c3ccc4ncccc4c3)c2c1] | 312 |
| 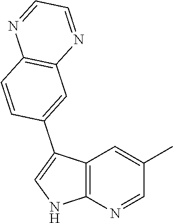 [SMILES: Cc1cnc2[nH]cc(-c3ccc4nccnc4c3)c2c1] | 313 |
| 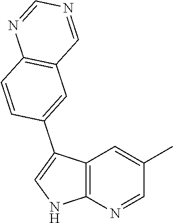 [SMILES: Cc1cnc2[nH]cc(-c3ccc4ncncc4c3)c2c1] | 314 |
| 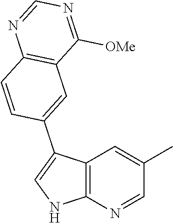 [SMILES: COc1ncnc2ccc(-c3c[nH]c4ncc(C)cc34)cc12] | 315 |
| 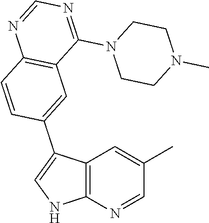 [SMILES: Cc1cnc2[nH]cc(-c3ccc4ncnc(N5CCN(C)CC5)c4c3)c2c1] | 316 |
| 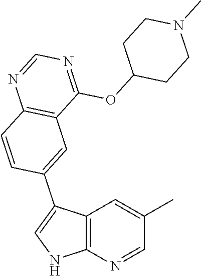 [SMILES: Cc1cnc2[nH]cc(-c3ccc4ncnc(OC5CCN(C)CC5)c4c3)c2c1] | 317 |
| 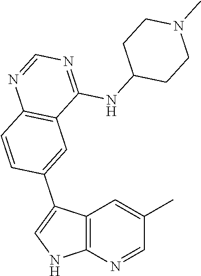 [SMILES: Cc1cnc2[nH]cc(-c3ccc4ncnc(NC5CCN(C)CC5)c4c3)c2c1] | 318 |
| 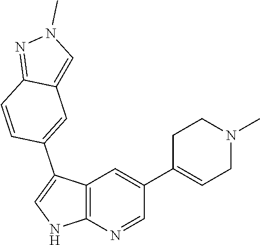 [SMILES: CN1CC=C(c2cnc3[nH]cc(-c4ccc5nn(C)cc5c4)c3c2)CC1] | 319 |
| 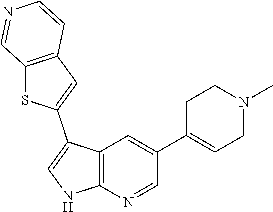 [SMILES: CN1CC=C(c2cnc3[nH]cc(-c4cc5ccncc5s4)c3c2)CC1] | 320 |
| 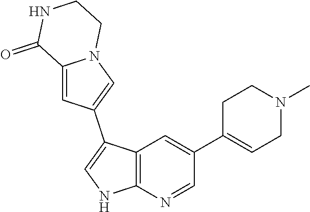 [SMILES: CN1CC=C(c2cnc3[nH]cc(-c4cc5n(c4)CCNC5=O)c3c2)CC1] | 321 |
| 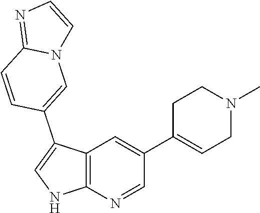 [SMILES: CN1CC=C(c2cnc3[nH]cc(-c4ccc5nccn5c4)c3c2)CC1] | 322 |
| 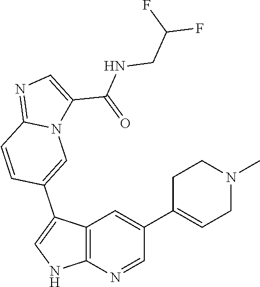 [SMILES: CN1CC=C(c2cnc3[nH]cc(-c4ccc5ncc(C(=O)NCC(F)F)n5c4)c3c2)CC1] | 323 |
| 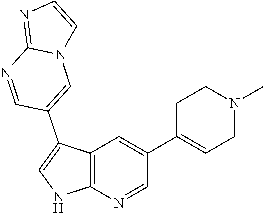 [SMILES: CN1CC=C(c2cnc3[nH]cc(-c4cnc5nccn5c4)c3c2)CC1] | 324 |
| 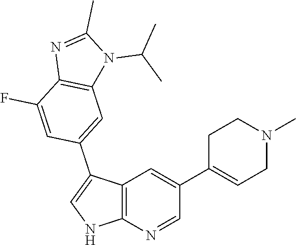 [SMILES: Cc1nc2c(F)cc(-c3c[nH]c4ncc(C5=CCN(C)CC5)cc34)cc2n1C(C)C] | 325 |
| 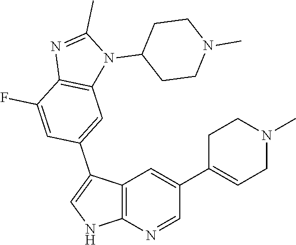 [SMILES: Cc1nc2c(F)cc(-c3c[nH]c4ncc(C5=CCN(C)CC5)cc34)cc2n1C1CCN(C)CC1] | 326 |
| 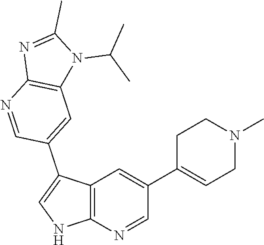 [SMILES: Cc1nc2ncc(-c3c[nH]c4ncc(C5=CCN(C)CC5)cc34)cc2n1C(C)C] | 327 |
| 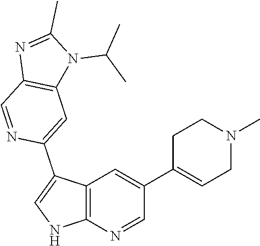 [SMILES: Cc1nc2cnc(-c3c[nH]c4ncc(C5=CCN(C)CC5)cc34)cc2n1C(C)C] | 328 |
| 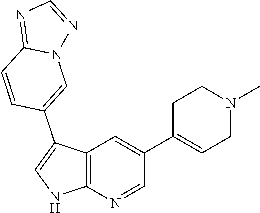 [SMILES: CN1CC=C(c2cnc3[nH]cc(-c4ccc5ncnn5c4)c3c2)CC1] | 329 |
| 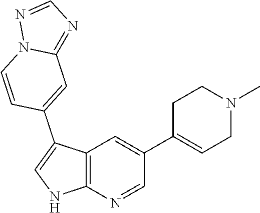 [SMILES: CN1CC=C(c2cnc3[nH]cc(-c4ccn5ncnc5c4)c3c2)CC1] | 330 |
| 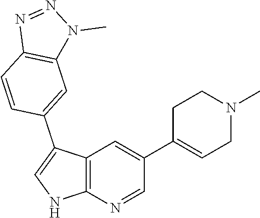 [SMILES: CN1CC=C(c2cnc3[nH]cc(-c4ccc5nnn(C)c5c4)c3c2)CC1] | 331 |
| 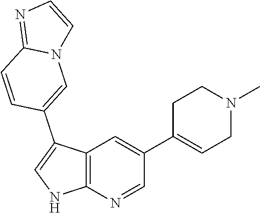 [SMILES: CN1CC=C(c2cnc3[nH]cc(-c4ccc5nccn5c4)c3c2)CC1] | 332 |
| 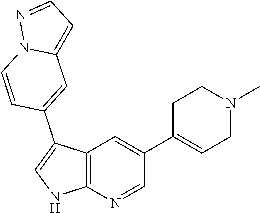 [SMILES: CN1CC=C(c2cnc3[nH]cc(-c4ccn5nccc5c4)c3c2)CC1] | 333 |
| 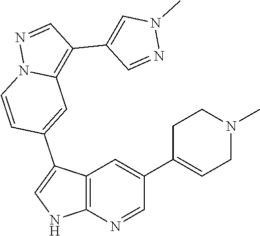 [SMILES: CN1CC=C(c2cnc3[nH]cc(-c4ccn5ncc(-c6cnn(C)c6)c5c4)c3c2)CC1] | 334 |
| 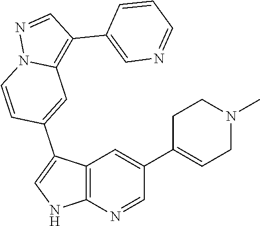 [SMILES: CN1CC=C(c2cnc3[nH]cc(-c4ccn5ncc(-c6cccnc6)c5c4)c3c2)CC1] | 335 |
| 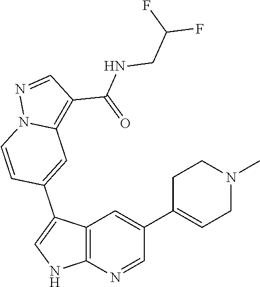 [SMILES: CN1CC=C(c2cnc3[nH]cc(-c4ccn5ncc(C(=O)NCC(F)F)c5c4)c3c2)CC1] | 336 |
| 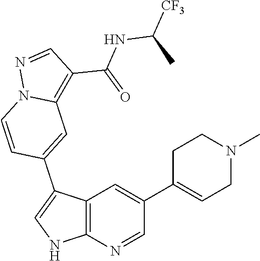 [SMILES: C[C@@H](NC(=O)c1cnn2ccc(-c3c[nH]c4ncc(C5=CCN(C)CC5)cc34)cc12)C(F)(F)F] | 337 |
| 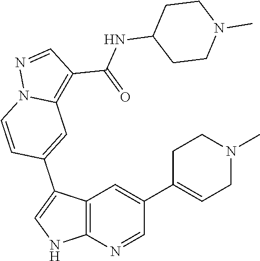 [SMILES: CN1CC=C(c2cnc3[nH]cc(-c4ccn5ncc(C(=O)NC6CCN(C)CC6)c5c4)c3c2)CC1] | 338 |
| 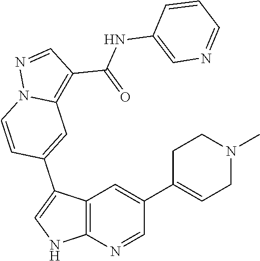 [SMILES: CN1CC=C(c2cnc3[nH]cc(-c4ccn5ncc(C(=O)Nc6cccnc6)c5c4)c3c2)CC1] | 339 |
| 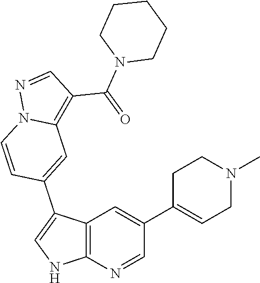 [SMILES: CN1CC=C(c2cnc3[nH]cc(-c4ccn5ncc(C(=O)N6CCCCC6)c5c4)c3c2)CC1] | 340 |
| 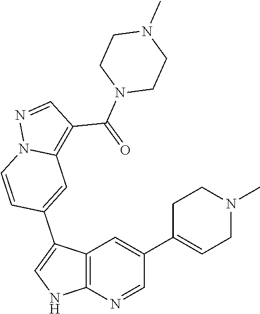 [SMILES: CN1CC=C(c2cnc3[nH]cc(-c4ccn5ncc(C(=O)N6CCN(C)CC6)c5c4)c3c2)CC1] | 341 |
| 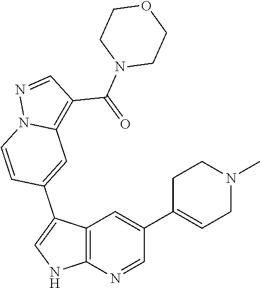 [SMILES: CN1CC=C(c2cnc3[nH]cc(-c4ccn5ncc(C(=O)N6CCOCC6)c5c4)c3c2)CC1] | 342 |
| 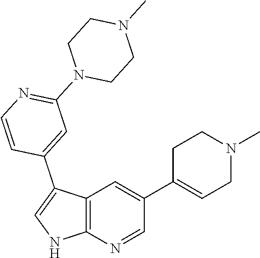 [SMILES: CN1CC=C(c2cnc3[nH]cc(-c4ccnc(N5CCN(C)CC5)c4)c3c2)CC1] | 343 |
| 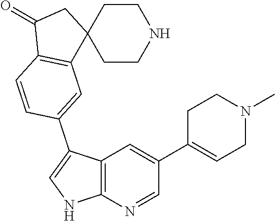 [SMILES: CN1CC=C(c2cnc3[nH]cc(-c4ccc5c(c4)C4(CCNCC4)CC5=O)c3c2)CC1] | 344 |
| 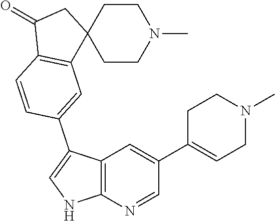 [SMILES: CN1CC=C(c2cnc3[nH]cc(-c4ccc5c(c4)C4(CCN(C)CC4)CC5=O)c3c2)CC1] | 345 |
| 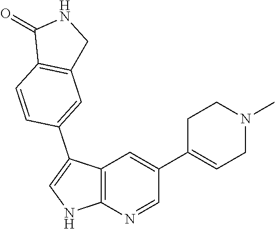 [SMILES: CN1CC=C(c2cnc3[nH]cc(-c4ccc5c(c4)CNC5=O)c3c2)CC1] | 346 |
| 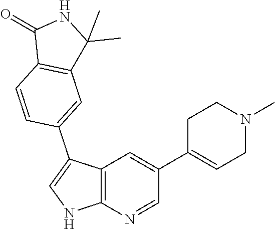 [SMILES: CN1CC=C(c2cnc3[nH]cc(-c4ccc5c(c4)C(C)(C)NC5=O)c3c2)CC1] | 347 |
| 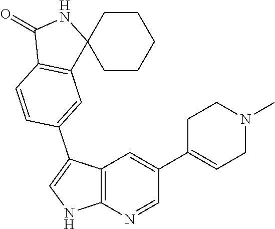 [SMILES: CN1CC=C(c2cnc3[nH]cc(-c4ccc5c(c4)C4(CCCCC4)NC5=O)c3c2)CC1] | 348 |
| 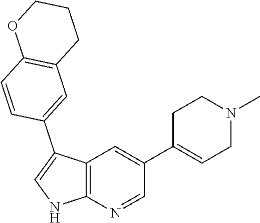 [SMILES: CN1CC=C(c2cnc3[nH]cc(-c4ccc5c(c4)CCCO5)c3c2)CC1] | 349 |
| 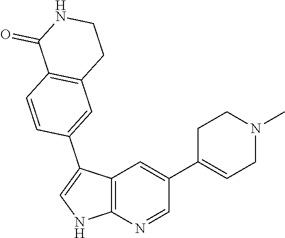 [SMILES: CN1CC=C(c2cnc3[nH]cc(-c4ccc5c(c4)CCNC5=O)c3c2)CC1] | 350 |
| 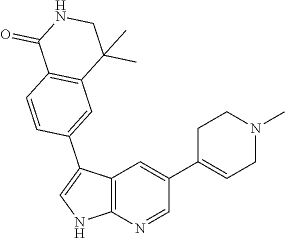 [SMILES: CN1CC=C(c2cnc3[nH]cc(-c4ccc5c(c4)C(C)(C)CNC5=O)c3c2)CC1] | 351 |
| 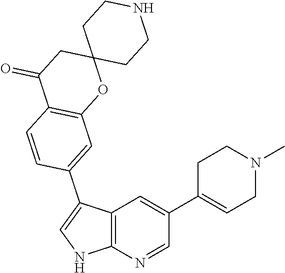 [SMILES: CN1CC=C(c2cnc3[nH]cc(-c4ccc5c(c4)OC4(CCNCC4)CC5=O)c3c2)CC1] | 352 |
| 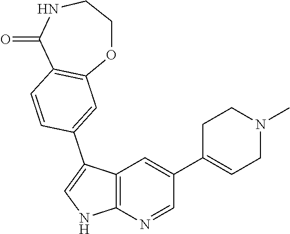 [SMILES: CN1CC=C(c2cnc3[nH]cc(-c4ccc5c(c4)OCCNC5=O)c3c2)CC1] | 353 |
| 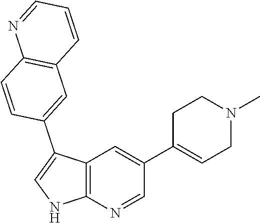 [SMILES: CN1CC=C(c2cnc3[nH]cc(-c4ccc5ncccc5c4)c3c2)CC1] | 354 |
| 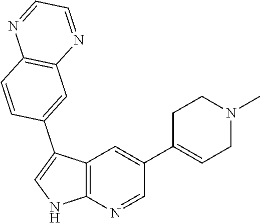 [SMILES: CN1CC=C(c2cnc3[nH]cc(-c4ccc5nccnc5c4)c3c2)CC1] | 355 |
| 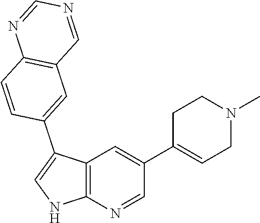 [SMILES: CN1CC=C(c2cnc3[nH]cc(-c4ccc5ncncc5c4)c3c2)CC1] | 356 |
| 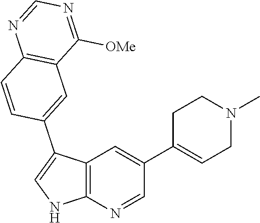 [SMILES: COc1ncnc2ccc(-c3c[nH]c4ncc(C5=CCN(C)CC5)cc34)cc12] | 357 |
| 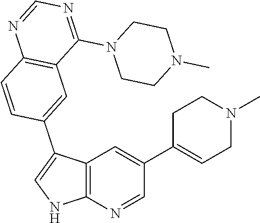 [SMILES: CN1CC=C(c2cnc3[nH]cc(-c4ccc5ncnc(N6CCN(C)CC6)c5c4)c3c2)CC1] | 358 |
| 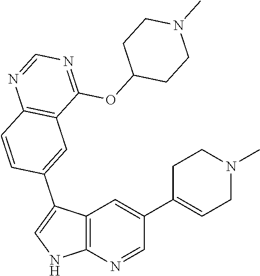 [SMILES: CN1CC=C(c2cnc3[nH]cc(-c4ccc5ncnc(OC6CCN(C)CC6)c5c4)c3c2)CC1] | 359 |
| 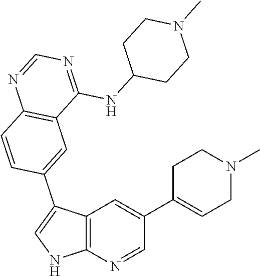 [SMILES: CN1CC=C(c2cnc3[nH]cc(-c4ccc5ncnc(NC6CCN(C)CC6)c5c4)c3c2)CC1] | 360 |
| 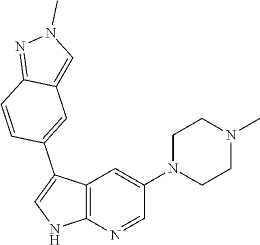 [SMILES: CN1CCN(c2cnc3[nH]cc(-c4ccc5nn(C)cc5c4)c3c2)CC1] | 361 |
| 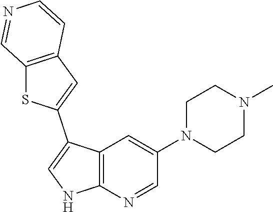 [SMILES: CN1CCN(c2cnc3[nH]cc(-c4cc5ccncc5s4)c3c2)CC1] | 362 |
| 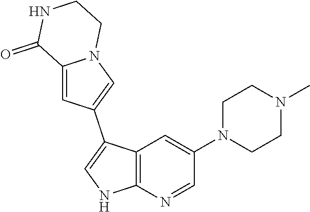 [SMILES: CN1CCN(c2cnc3[nH]cc(-c4cc5n(c4)CCNC5=O)c3c2)CC1] | 363 |
| 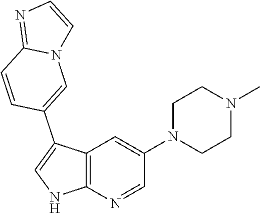 [SMILES: CN1CCN(c2cnc3[nH]cc(-c4ccc5nccn5c4)c3c2)CC1] | 364 |
| 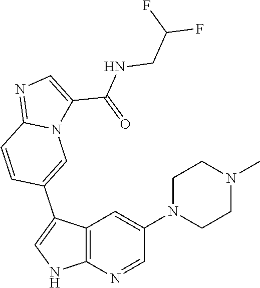 [SMILES: CN1CCN(c2cnc3[nH]cc(-c4ccc5ncc(C(=O)NCC(F)F)n5c4)c3c2)CC1] | 365 |
| 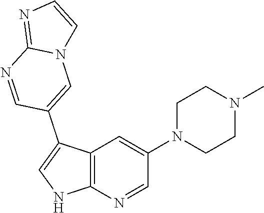 [SMILES: CN1CCN(c2cnc3[nH]cc(-c4cnc5nccn5c4)c3c2)CC1] | 366 |
| 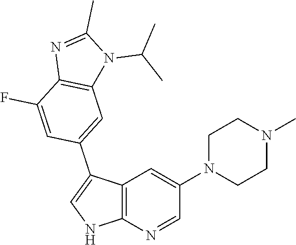 [SMILES: Cc1nc2c(F)cc(-c3c[nH]c4ncc(N5CCN(C)CC5)cc34)cc2n1C(C)C] | 367 |
| 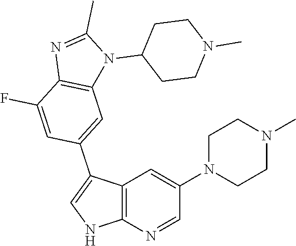 [SMILES: Cc1nc2c(F)cc(-c3c[nH]c4ncc(N5CCN(C)CC5)cc34)cc2n1C1CCN(C)CC1] | 368 |
| 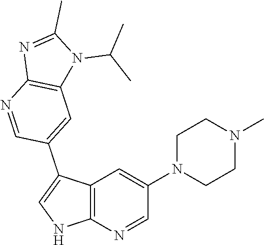 [SMILES: Cc1nc2ncc(-c3c[nH]c4ncc(N5CCN(C)CC5)cc34)cc2n1C(C)C] | 369 |
| 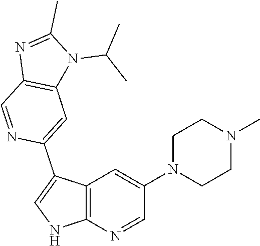 [SMILES: Cc1nc2cnc(-c3c[nH]c4ncc(N5CCN(C)CC5)cc34)cc2n1C(C)C] | 370 |
| 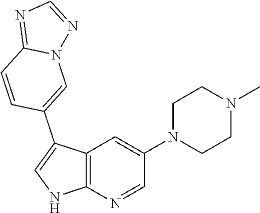 [SMILES: CN1CCN(c2cnc3[nH]cc(-c4ccc5ncnn5c4)c3c2)CC1] | 371 |
| 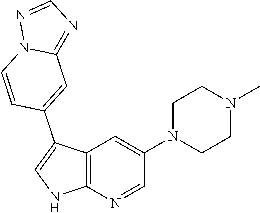 [SMILES: CN1CCN(c2cnc3[nH]cc(-c4ccn5ncnc5c4)c3c2)CC1] | 372 |
| 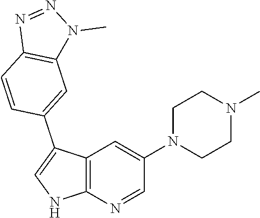 [SMILES: CN1CCN(c2cnc3[nH]cc(-c4ccc5nnn(C)c5c4)c3c2)CC1] | 373 |
| 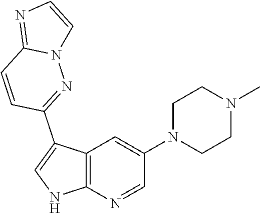 [SMILES: CN1CCN(c2cnc3[nH]cc(-c4ccc5nccn5n4)c3c2)CC1] | 374 |
| 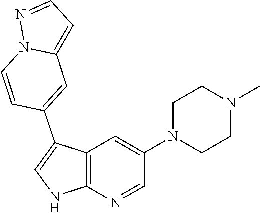 [SMILES: CN1CCN(c2cnc3[nH]cc(-c4ccn5nccc5c4)c3c2)CC1] | 375 |
| 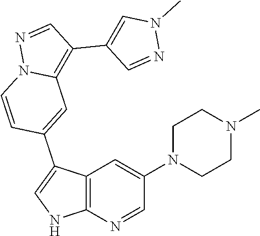 [SMILES: CN1CCN(c2cnc3[nH]cc(-c4ccn5ncc(-c6cnn(C)c6)c5c4)c3c2)CC1] | 376 |
| 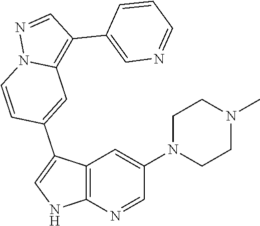 [SMILES: CN1CCN(c2cnc3[nH]cc(-c4ccn5ncc(-c6cccnc6)c5c4)c3c2)CC1] | 377 |
| 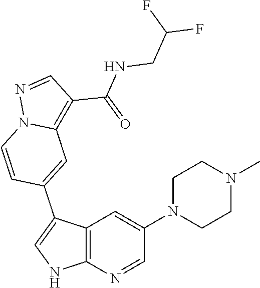 [SMILES: CN1CCN(c2cnc3[nH]cc(-c4ccn5ncc(C(=O)NCC(F)F)c5c4)c3c2)CC1] | 378 |
| 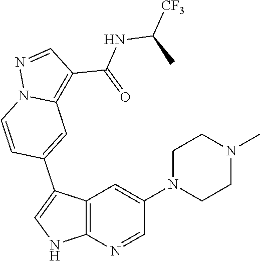 [SMILES: C[C@@H](NC(=O)c1cnn2ccc(-c3c[nH]c4ncc(N5CCN(C)CC5)cc34)cc12)C(F)(F)F] | 379 |
| 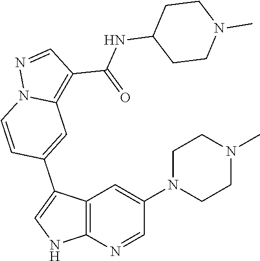 [SMILES: CN1CCC(NC(=O)c2cnn3ccc(-c4c[nH]c5ncc(N6CCN(C)CC6)cc45)cc23)CC1] | 380 |
| 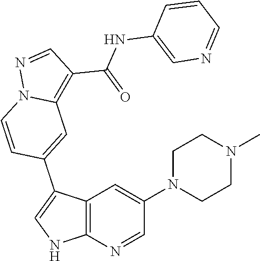 [SMILES: CN1CCN(c2cnc3[nH]cc(-c4ccn5ncc(C(=O)Nc6cccnc6)c5c4)c3c2)CC1] | 381 |
| 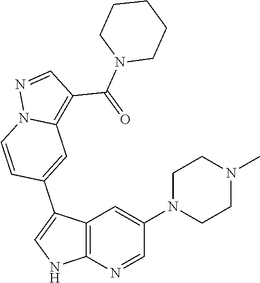 [SMILES: CN1CCN(c2cnc3[nH]cc(-c4ccn5ncc(C(=O)N6CCCCC6)c5c4)c3c2)CC1] | 382 |
| 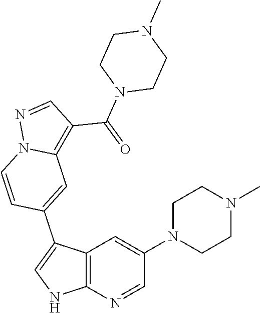 [SMILES: CN1CCN(C(=O)c2cnn3ccc(-c4c[nH]c5ncc(N6CCN(C)CC6)cc45)cc23)CC1] | 383 |
| 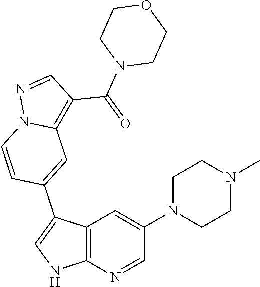 [SMILES: CN1CCN(c2cnc3[nH]cc(-c4ccn5ncc(C(=O)N6CCOCC6)c5c4)c3c2)CC1] | 384 |
| 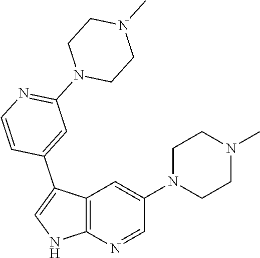 [SMILES: CN1CCN(c2cnc3[nH]cc(-c4ccnc(N5CCN(C)CC5)c4)c3c2)CC1] | 385 |
| 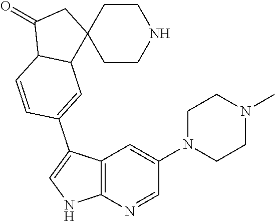 [SMILES: CN1CCN(c2cnc3[nH]cc(C4=CC5C(C=C4)C(=O)CC54CCNCC4)c3c2)CC1] | 386 |
| 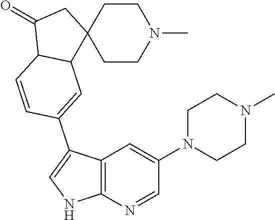 [SMILES: CN1CCN(c2cnc3[nH]cc(C4=CC5C(C=C4)C(=O)CC54CCN(C)CC4)c3c2)CC1] | 387 |
| 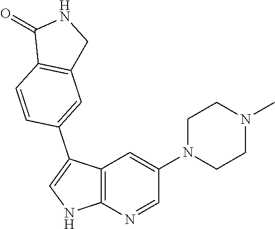 [SMILES: CN1CCN(c2cnc3[nH]cc(-c4ccc5c(c4)CNC5=O)c3c2)CC1] | 388 |
| 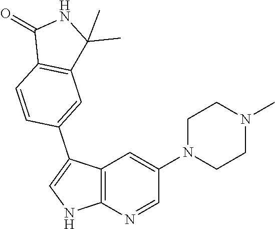 [SMILES: CN1CCN(c2cnc3[nH]cc(-c4ccc5c(c4)C(C)(C)NC5=O)c3c2)CC1] | 389 |
| 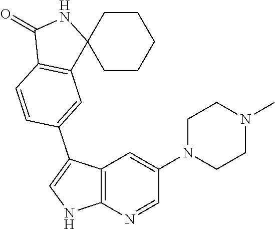 [SMILES: CN1CCN(c2cnc3[nH]cc(-c4ccc5c(c4)C4(CCCCC4)NC5=O)c3c2)CC1] | 390 |
| 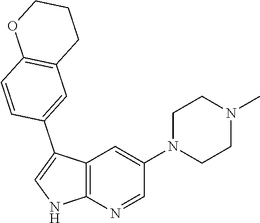 [SMILES: CN1CCN(c2cnc3[nH]cc(-c4ccc5c(c4)CCCO5)c3c2)CC1] | 391 |
| 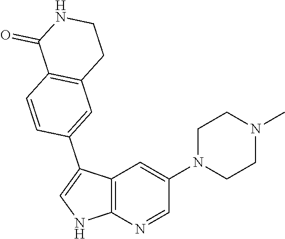 [SMILES: CN1CCN(c2cnc3[nH]cc(-c4ccc5c(c4)CCNC5=O)c3c2)CC1] | 392 |
| 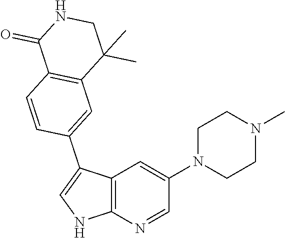 [SMILES: CN1CCN(c2cnc3[nH]cc(-c4ccc5c(c4)C(C)(C)CNC5=O)c3c2)CC1] | 393 |
| 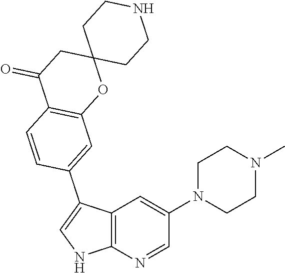 [SMILES: CN1CCN(c2cnc3[nH]cc(-c4ccc5c(c4)OC4(CCNCC4)CC5=O)c3c2)CC1] | 394 |
| 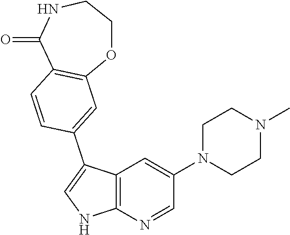 [SMILES: CN1CCN(c2cnc3[nH]cc(-c4ccc5c(c4)OCCNC5=O)c3c2)CC1] | 395 |
| 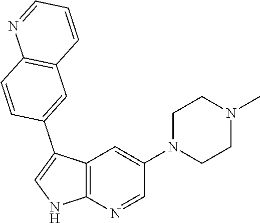 [SMILES: CN1CCN(c2cnc3[nH]cc(-c4ccc5ncccc5c4)c3c2)CC1] | 396 |
| 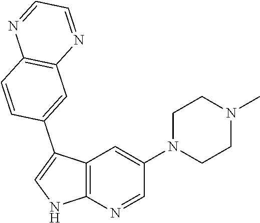 [SMILES: CN1CCN(c2cnc3[nH]cc(-c4ccc5nccnc5c4)c3c2)CC1] | 397 |
| 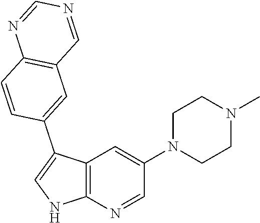 [SMILES: CN1CCN(c2cnc3[nH]cc(-c4ccc5ncncc5c4)c3c2)CC1] | 398 |
| 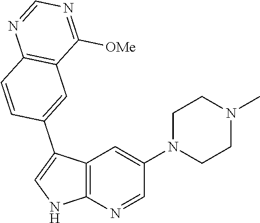 [SMILES: COc1ncnc2ccc(-c3c[nH]c4ncc(N5CCN(C)CC5)cc34)cc12] | 399 |
| 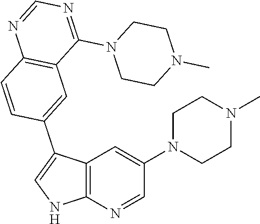 [SMILES: CN1CCN(c2cnc3[nH]cc(-c4ccc5ncnc(N6CCN(C)CC6)c5c4)c3c2)CC1] | 400 |
| 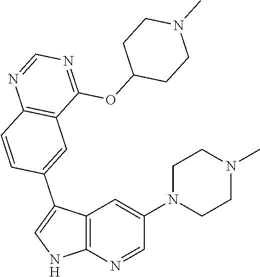 [SMILES: CN1CCC(Oc2ncnc3ccc(-c4c[nH]c5ncc(N6CCN(C)CC6)cc45)cc23)CC1] | 401 |
| 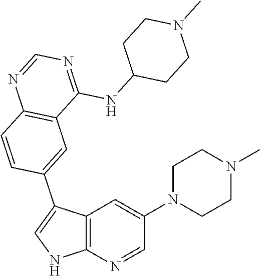 [SMILES: CN1CCC(Nc2ncnc3ccc(-c4c[nH]c5ncc(N6CCN(C)CC6)cc45)cc23)CC1] | 402 |
| 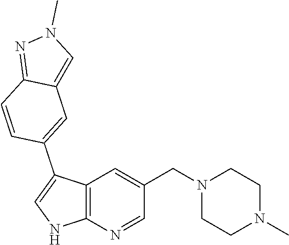 [SMILES: CN1CCN(Cc2cnc3[nH]cc(-c4ccc5nn(C)cc5c4)c3c2)CC1] | 403 |
| 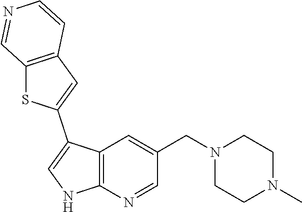 [SMILES: CN1CCN(Cc2cnc3[nH]cc(-c4cc5ccncc5s4)c3c2)CC1] | 404 |
| 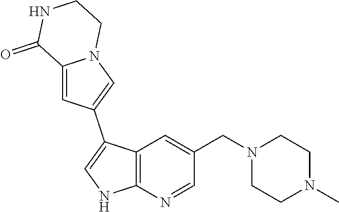 [SMILES: CN1CCN(Cc2cnc3[nH]cc(-c4cc5n(c4)CCNC5=O)c3c2)CC1] | 405 |
| 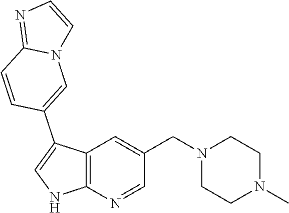 [SMILES: CN1CCN(Cc2cnc3[nH]cc(-c4ccc5nccn5c4)c3c2)CC1] | 406 |
| 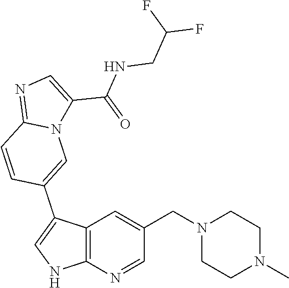 [SMILES: CN1CCN(Cc2cnc3[nH]cc(-c4ccc5ncc(C(=O)NCC(F)F)n5c4)c3c2)CC1] | 407 |
| 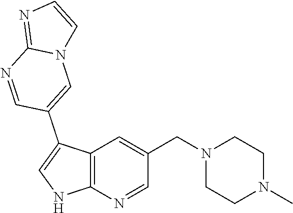 [SMILES: CN1CCN(Cc2cnc3[nH]cc(-c4cnc5nccn5c4)c3c2)CC1] | 408 |
| 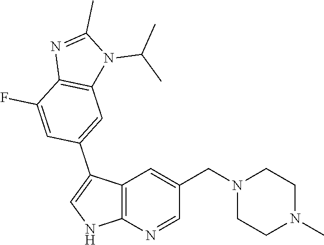 [SMILES: Cc1nc2c(F)cc(-c3c[nH]c4ncc(CN5CCN(C)CC5)cc34)cc2n1C(C)C] | 409 |
| 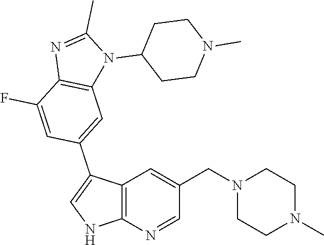 [SMILES: Cc1nc2c(F)cc(-c3c[nH]c4ncc(CN5CCN(C)CC5)cc34)cc2n1C1CCN(C)CC1] | 410 |
| 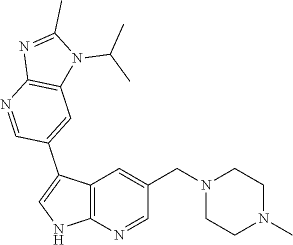 [SMILES: Cc1nc2ncc(-c3c[nH]c4ncc(CN5CCN(C)CC5)cc34)cc2n1C(C)C] | 411 |
| 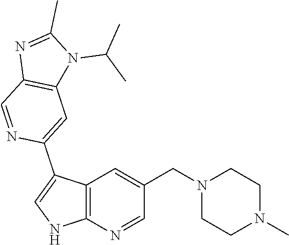 [SMILES: Cc1nc2cnc(-c3c[nH]c4ncc(CN5CCN(C)CC5)cc34)cc2n1C(C)C] | 412 |
| 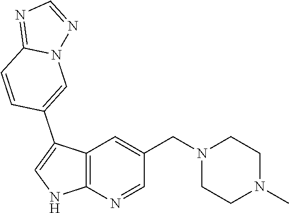 [SMILES: CN1CCN(Cc2cnc3[nH]cc(-c4ccc5ncnn5c4)c3c2)CC1] | 413 |
| 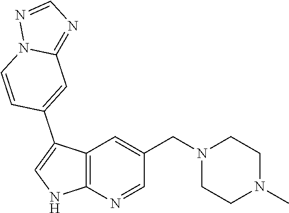 [SMILES: CN1CCN(Cc2cnc3[nH]cc(-c4ccn5ncnc5c4)c3c2)CC1] | 414 |
| 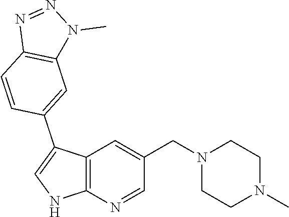 [SMILES: CN1CCN(Cc2cnc3[nH]cc(-c4ccc5nnn(C)c5c4)c3c2)CC1] | 415 |
| 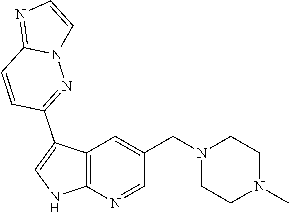 [SMILES: CN1CCN(Cc2cnc3[nH]cc(-c4ccc5nccn5n4)c3c2)CC1] | 416 |
| 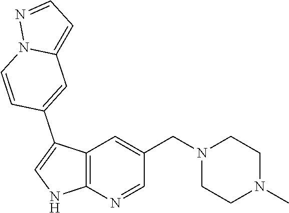 [SMILES: CN1CCN(Cc2cnc3[nH]cc(-c4ccn5nccc5c4)c3c2)CC1] | 417 |
| 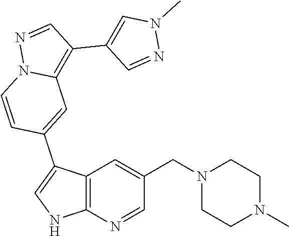 [SMILES: CN1CCN(Cc2cnc3[nH]cc(-c4ccn5ncc(-c6cnn(C)c6)c5c4)c3c2)CC1] | 418 |
| 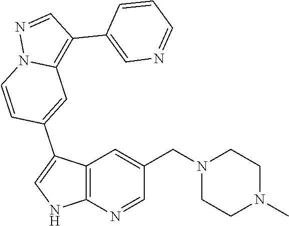 [SMILES: CN1CCN(Cc2cnc3[nH]cc(-c4ccn5ncc(-c6cccnc6)c5c4)c3c2)CC1] | 419 |
| 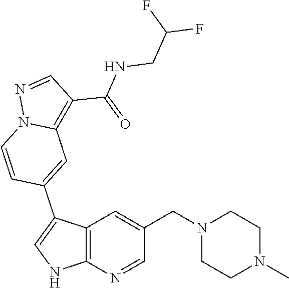 [SMILES: CN1CCN(Cc2cnc3[nH]cc(-c4ccn5ncc(C(=O)NCC(F)F)c5c4)c3c2)CC1] | 420 |
| 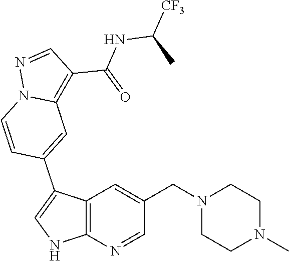 [SMILES: C[C@@H](NC(=O)c1cnn2ccc(-c3c[nH]c4ncc(CN5CCN(C)CC5)cc34)cc12)C(F)(F)F] | 421 |
| 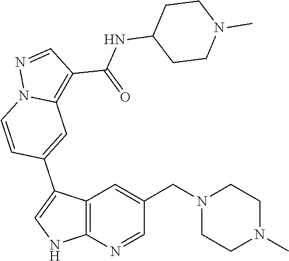 [SMILES: CN1CCC(NC(=O)c2cnn3ccc(-c4c[nH]c5ncc(CN6CCN(C)CC6)cc45)cc23)CC1] | 422 |
| 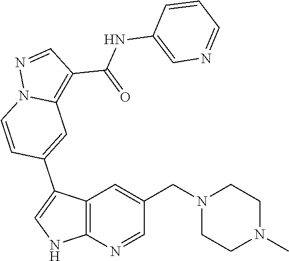 [SMILES: CN1CCN(Cc2cnc3[nH]cc(-c4ccn5ncc(C(=O)Nc6cccnc6)c5c4)c3c2)CC1] | 423 |
| 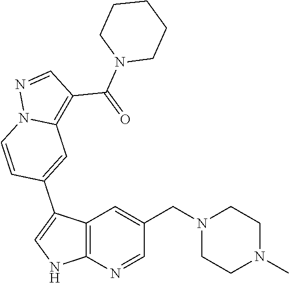 [SMILES: CN1CCN(Cc2cnc3[nH]cc(-c4ccn5ncc(C(=O)N6CCCCC6)c5c4)c3c2)CC1] | 424 |
| 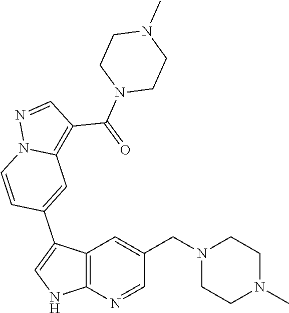 [SMILES: CN1CCN(Cc2cnc3[nH]cc(-c4ccn5ncc(C(=O)N6CCN(C)CC6)c5c4)c3c2)CC1] | 425 |
| 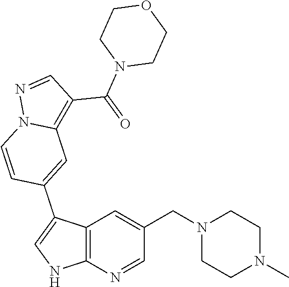 [SMILES: CN1CCN(Cc2cnc3[nH]cc(-c4ccn5ncc(C(=O)N6CCOCC6)c5c4)c3c2)CC1] | 426 |
| 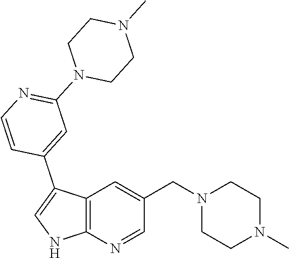 [SMILES: CN1CCN(Cc2cnc3[nH]cc(-c4ccnc(N5CCN(C)CC5)c4)c3c2)CC1] | 427 |
| 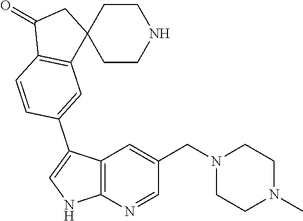 [SMILES: CN1CCN(Cc2cnc3[nH]cc(-c4ccc5c(c4)C4(CCNCC4)CC5=O)c3c2)CC1] | 428 |
| 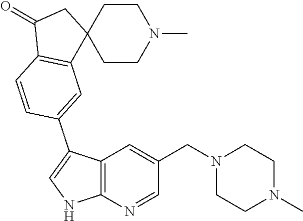 [SMILES: CN1CCN(Cc2cnc3[nH]cc(-c4ccc5c(c4)C4(CCN(C)CC4)CC5=O)c3c2)CC1] | 429 |
| 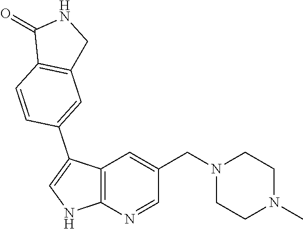 [SMILES: CN1CCN(Cc2cnc3[nH]cc(-c4ccc5c(c4)CNC5=O)c3c2)CC1] | 430 |
| 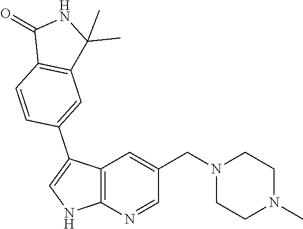 [SMILES: CN1CCN(Cc2cnc3[nH]cc(-c4ccc5c(c4)C(C)(C)NC5=O)c3c2)CC1] | 431 |
| 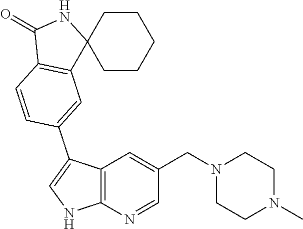 [SMILES: CN1CCN(Cc2cnc3[nH]cc(-c4ccc5c(c4)C4(CCCCC4)NC5=O)c3c2)CC1] | 432 |
| 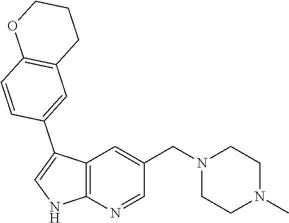 [SMILES: CN1CCN(Cc2cnc3[nH]cc(-c4ccc5c(c4)CCCO5)c3c2)CC1] | 433 |
| 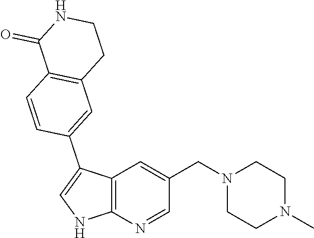 [SMILES: CN1CCN(Cc2cnc3[nH]cc(-c4ccc5c(c4)CCNC5=O)c3c2)CC1] | 434 |
| 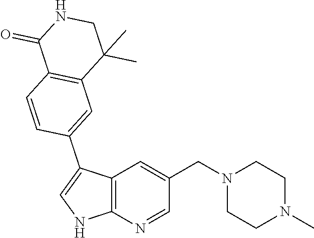 [SMILES: CN1CCN(Cc2cnc3[nH]cc(-c4ccc5c(c4)C(C)(C)CNC5=O)c3c2)CC1] | 435 |
| 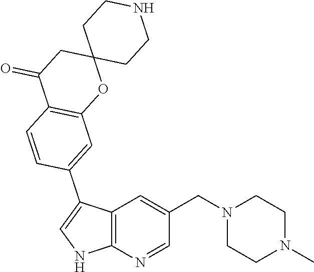 [SMILES: CN1CCN(Cc2cnc3[nH]cc(-c4ccc5c(c4)OC4(CCNCC4)CC5=O)c3c2)CC1] | 436 |
| 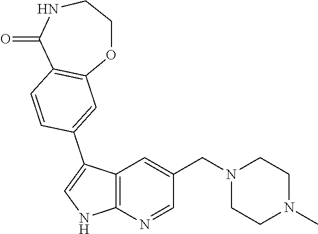 [SMILES: CN1CCN(Cc2cnc3[nH]cc(-c4ccc5c(c4)OCCNC5=O)c3c2)CC1] | 437 |
| 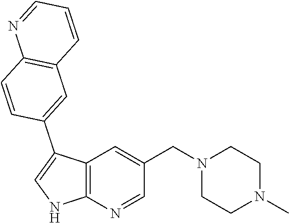 [SMILES: CN1CCN(Cc2cnc3[nH]cc(-c4ccc5ncccc5c4)c3c2)CC1] | 438 |
| 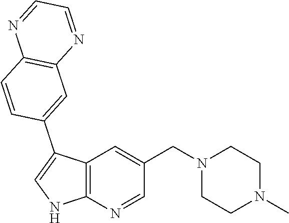 [SMILES: CN1CCN(Cc2cnc3[nH]cc(-c4ccc5nccnc5c4)c3c2)CC1] | 439 |
| 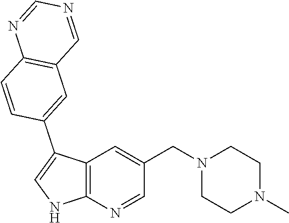 [SMILES: CN1CCN(Cc2cnc3[nH]cc(-c4ccc5ncncc5c4)c3c2)CC1] | 440 |
| 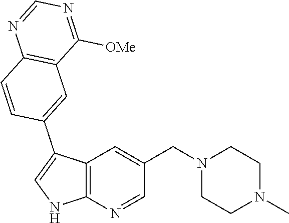 [SMILES: COc1ncnc2ccc(-c3c[nH]c4ncc(CN5CCN(C)CC5)cc34)cc12] | 441 |
| 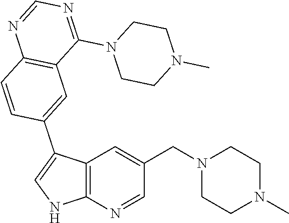 [SMILES: CN1CCN(Cc2cnc3[nH]cc(-c4ccc5ncnc(N6CCN(C)CC6)c5c4)c3c2)CC1] | 442 |
| 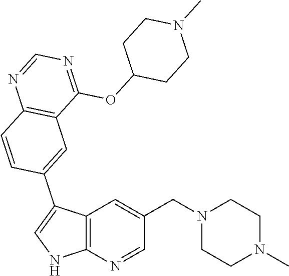 [SMILES: CN1CCC(Oc2ncnc3ccc(-c4c[nH]c5ncc(CN6CCN(C)CC6)cc45)cc23)CC1] | 443 |
| 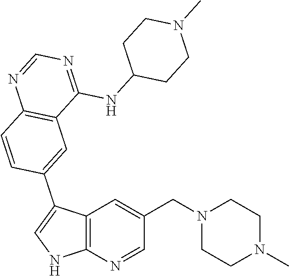 [SMILES: CN1CCC(Nc2ncnc3ccc(-c4c[nH]c5ncc(CN6CCN(C)CC6)cc45)cc23)CC1] | 444 |
| 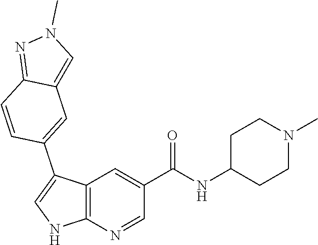 [SMILES: CN1CCC(NC(=O)c2cnc3[nH]cc(-c4ccc5nn(C)cc5c4)c3c2)CC1] | 445 |
| 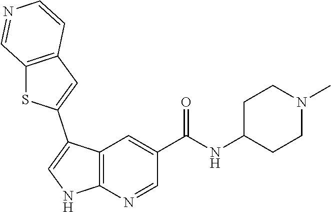 [SMILES: CN1CCC(NC(=O)c2cnc3[nH]cc(-c4cc5ccncc5s4)c3c2)CC1] | 446 |
| 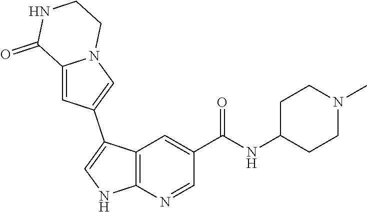 [SMILES: CN1CCC(NC(=O)c2cnc3[nH]cc(-c4cc5n(c4)CCNC5=O)c3c2)CC1] | 447 |
| 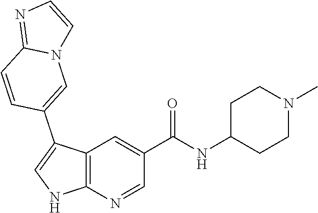 [SMILES: CN1CCC(NC(=O)c2cnc3[nH]cc(-c4ccc5nccn5c4)c3c2)CC1] | 448 |
| 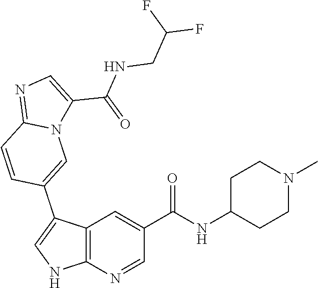 [SMILES: CN1CCC(NC(=O)c2cnc3[nH]cc(-c4ccc5ncc(C(=O)NCC(F)F)n5c4)c3c2)CC1] | 449 |
| 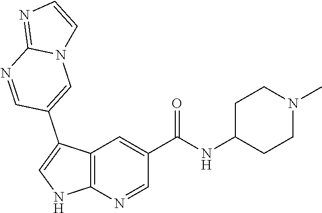 [SMILES: CN1CCC(NC(=O)c2cnc3[nH]cc(-c4cnc5nccn5c4)c3c2)CC1] | 450 |
| 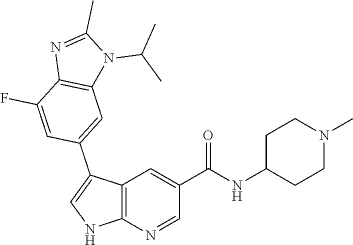 [SMILES: Cc1nc2c(F)cc(-c3c[nH]c4ncc(C(=O)NC5CCN(C)CC5)cc34)cc2n1C(C)C] | 451 |
| 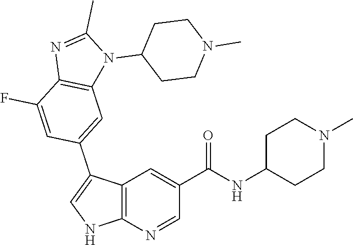 [SMILES: Cc1nc2c(F)cc(-c3c[nH]c4ncc(C(=O)NC5CCN(C)CC5)cc34)cc2n1C1CCN(C)CC1] | 452 |
| 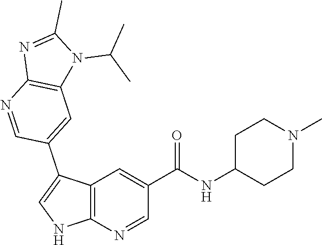 [SMILES: Cc1nc2ncc(-c3c[nH]c4ncc(C(=O)NC5CCN(C)CC5)cc34)cc2n1C(C)C] | 453 |
| 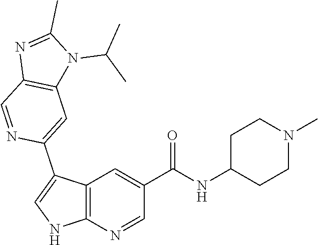 [SMILES: Cc1nc2cnc(-c3c[nH]c4ncc(C(=O)NC5CCN(C)CC5)cc34)cc2n1C(C)C] | 454 |
| 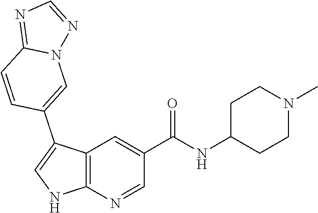 [SMILES: CN1CCC(NC(=O)c2cnc3[nH]cc(-c4ccc5ncnn5c4)c3c2)CC1] | 455 |
| 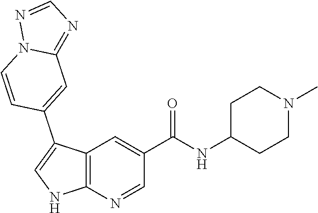 [SMILES: CN1CCC(NC(=O)c2cnc3[nH]cc(-c4ccn5ncnc5c4)c3c2)CC1] | 456 |
| 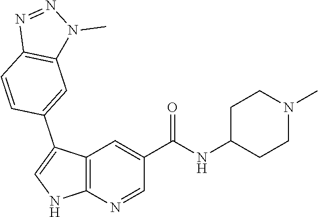 [SMILES: CN1CCC(NC(=O)c2cnc3[nH]cc(-c4ccc5nnn(C)c5c4)c3c2)CC1] | 457 |
| 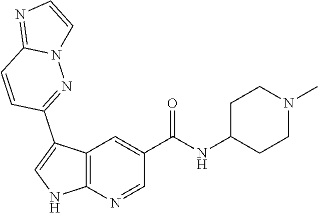 [SMILES: CN1CCC(NC(=O)c2cnc3[nH]cc(-c4ccc5nccn5n4)c3c2)CC1] | 458 |
| 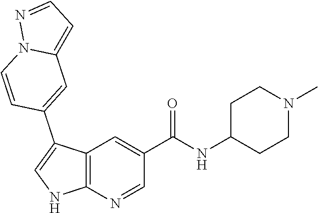 [SMILES: CN1CCC(NC(=O)c2cnc3[nH]cc(-c4ccn5nccc5c4)c3c2)CC1] | 459 |
| 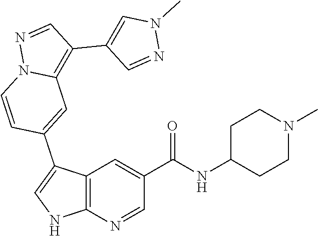 [SMILES: CN1CCC(NC(=O)c2cnc3[nH]cc(-c4ccn5ncc(-c6cnn(C)c6)c5c4)c3c2)CC1] | 460 |
| 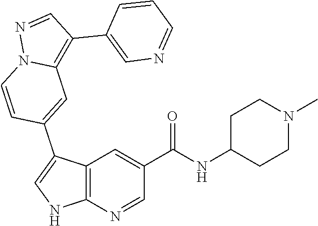 [SMILES: CN1CCC(NC(=O)c2cnc3[nH]cc(-c4ccn5ncc(-c6cccnc6)c5c4)c3c2)CC1] | 461 |
| 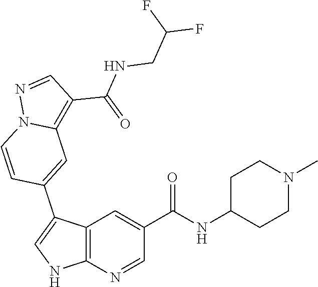 [SMILES: CN1CCC(NC(=O)c2cnc3[nH]cc(-c4ccn5ncc(C(=O)NCC(F)F)c5c4)c3c2)CC1] | 462 |
| 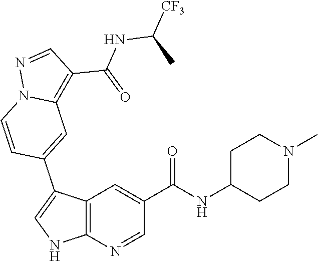 [SMILES: C[C@@H](NC(=O)c1cnn2ccc(-c3c[nH]c4ncc(C(=O)NC5CCN(C)CC5)cc34)cc12)C(F)(F)F] | 463 |
| 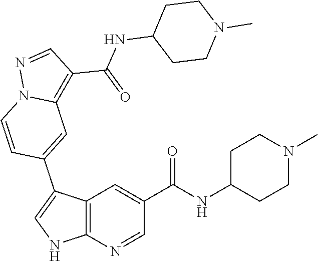 [SMILES: CN1CCC(NC(=O)c2cnc3[nH]cc(-c4ccn5ncc(C(=O)NC6CCN(C)CC6)c5c4)c3c2)CC1] | 464 |
| 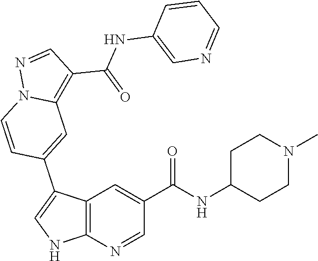 [SMILES: CN1CCC(NC(=O)c2cnc3[nH]cc(-c4ccn5ncc(C(=O)Nc6cccnc6)c5c4)c3c2)CC1] | 465 |
| 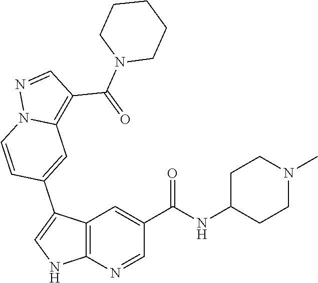 [SMILES: CN1CCC(NC(=O)c2cnc3[nH]cc(-c4ccn5ncc(C(=O)N6CCCCC6)c5c4)c3c2)CC1] | 466 |
| 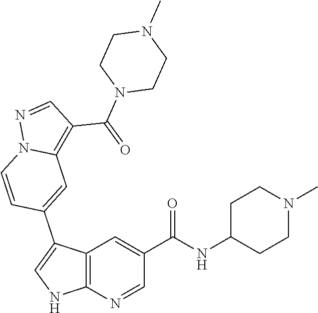 [SMILES: CN1CCC(NC(=O)c2cnc3[nH]cc(-c4ccn5ncc(C(=O)N6CCN(C)CC6)c5c4)c3c2)CC1] | 467 |
| 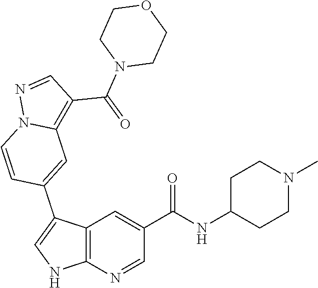 [SMILES: CN1CCC(NC(=O)c2cnc3[nH]cc(-c4ccn5ncc(C(=O)N6CCOCC6)c5c4)c3c2)CC1] | 468 |
| 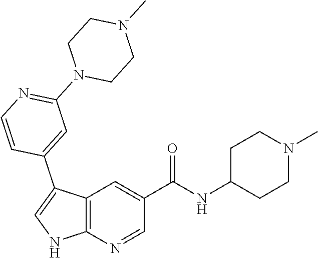 [SMILES: CN1CCC(NC(=O)c2cnc3[nH]cc(-c4ccnc(N5CCN(C)CC5)c4)c3c2)CC1] | 469 |
| 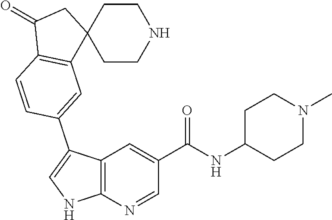 [SMILES: CN1CCC(NC(=O)c2cnc3[nH]cc(-c4ccc5c(c4)C4(CCNCC4)CC5=O)c3c2)CC1] | 470 |
| 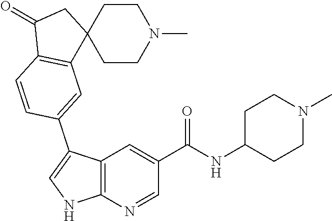 [SMILES: CN1CCC(NC(=O)c2cnc3[nH]cc(-c4ccc5c(c4)C4(CCN(C)CC4)CC5=O)c3c2)CC1] | 471 |
| 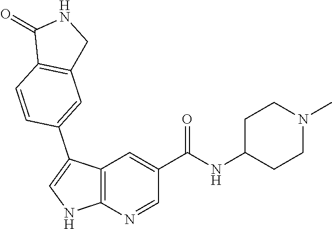 [SMILES: CN1CCC(NC(=O)c2cnc3[nH]cc(-c4ccc5c(c4)CNC5=O)c3c2)CC1] | 472 |
| 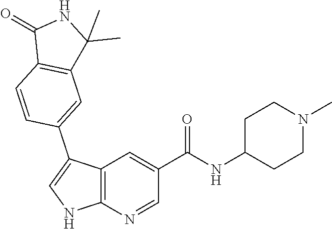 [SMILES: CN1CCC(NC(=O)c2cnc3[nH]cc(-c4ccc5c(c4)C(C)(C)NC5=O)c3c2)CC1] | 473 |
| 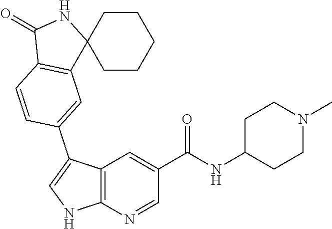 [SMILES: CN1CCC(NC(=O)c2cnc3[nH]cc(-c4ccc5c(c4)C4(CCCCC4)NC5=O)c3c2)CC1] | 474 |
| 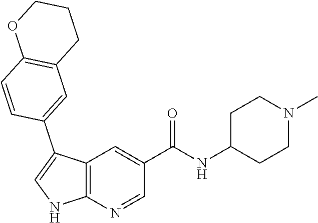 [SMILES: CN1CCC(NC(=O)c2cnc3[nH]cc(-c4ccc5c(c4)CCCO5)c3c2)CC1] | 475 |
| 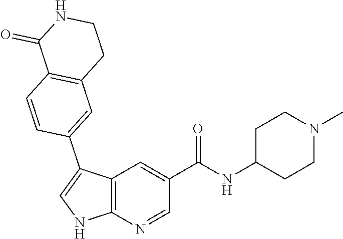 [SMILES: CN1CCC(NC(=O)c2cnc3[nH]cc(-c4ccc5c(c4)CCNC5=O)c3c2)CC1] | 476 |
| 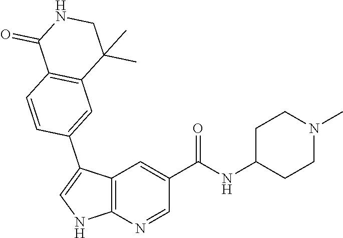 [SMILES: CN1CCC(NC(=O)c2cnc3[nH]cc(-c4ccc5c(c4)C(C)(C)CNC5=O)c3c2)CC1] | 477 |
| 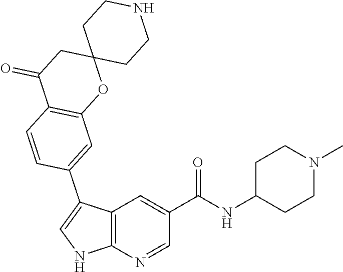 [SMILES: CN1CCC(NC(=O)c2cnc3[nH]cc(-c4ccc5c(c4)OC4(CCNCC4)CC5=O)c3c2)CC1] | 478 |
| 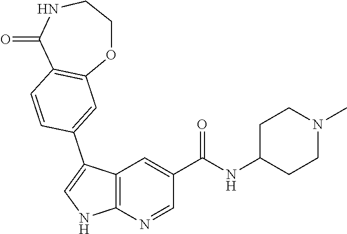 [SMILES: CN1CCC(NC(=O)c2cnc3[nH]cc(-c4ccc5c(c4)OCCNC5=O)c3c2)CC1] | 479 |
| 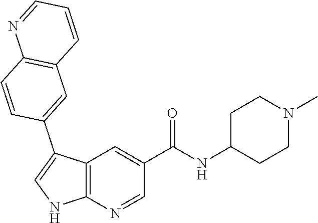 [SMILES: CN1CCC(NC(=O)c2cnc3[nH]cc(-c4ccc5ncccc5c4)c3c2)CC1] | 480 |
| 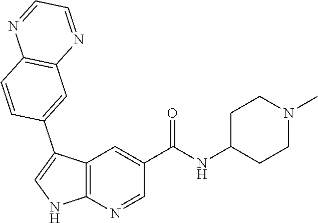 [SMILES: CN1CCC(NC(=O)c2cnc3[nH]cc(-c4ccc5nccnc5c4)c3c2)CC1] | 481 |
| 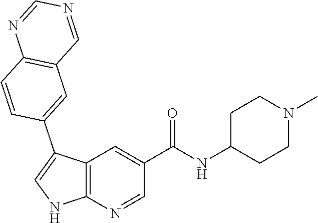 [SMILES: CN1CCC(NC(=O)c2cnc3[nH]cc(-c4ccc5ncncc5c4)c3c2)CC1] | 482 |
| 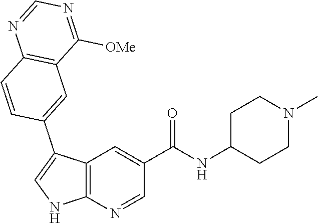 [SMILES: COc1ncnc2ccc(-c3c[nH]c4ncc(C(=O)NC5CCN(C)CC5)cc34)cc12] | 483 |
| 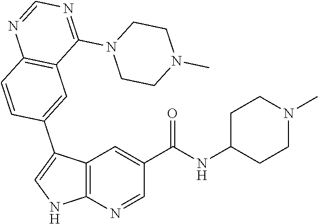 [SMILES: CN1CCC(NC(=O)c2cnc3[nH]cc(-c4ccc5ncnc(N6CCN(C)CC6)c5c4)c3c2)CC1] | 484 |
| 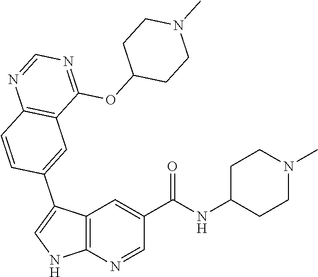 [SMILES: CN1CCC(NC(=O)c2cnc3[nH]cc(-c4ccc5ncnc(OC6CCN(C)CC6)c5c4)c3c2)CC1] | 485 |
| 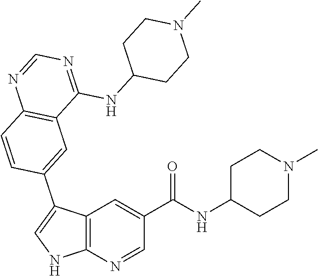 [SMILES: CN1CCC(NC(=O)c2cnc3[nH]cc(-c4ccc5ncnc(NC6CCN(C)CC6)c5c4)c3c2)CC1] | 486 |
| 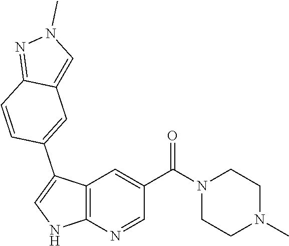 [SMILES: CN1CCN(C(=O)c2cnc3[nH]cc(-c4ccc5nn(C)cc5c4)c3c2)CC1] | 487 |
| 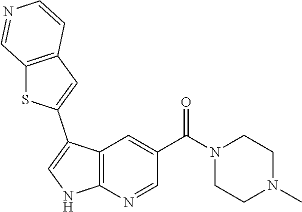 [SMILES: CN1CCN(C(=O)c2cnc3[nH]cc(-c4cc5ccncc5s4)c3c2)CC1] | 488 |
| 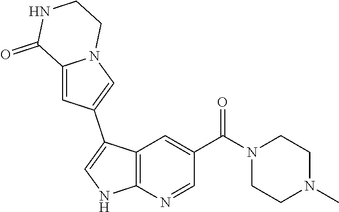 [SMILES: CN1CCN(C(=O)c2cnc3[nH]cc(-c4cc5n(c4)CCNC5=O)c3c2)CC1] | 489 |
| 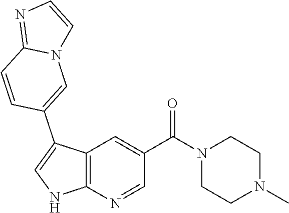 [SMILES: CN1CCN(C(=O)c2cnc3[nH]cc(-c4ccc5nccn5c4)c3c2)CC1] | 490 |
| 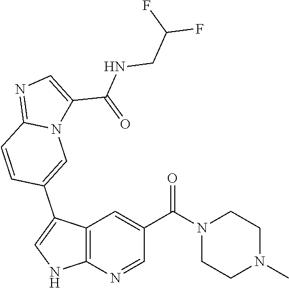 [SMILES: CN1CCN(C(=O)c2cnc3[nH]cc(-c4ccc5ncc(C(=O)NCC(F)F)n5c4)c3c2)CC1] | 491 |
| 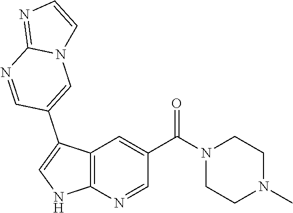 [SMILES: CN1CCN(C(=O)c2cnc3[nH]cc(-c4cnc5nccn5c4)c3c2)CC1] | 492 |
| 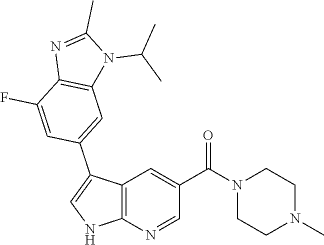 [SMILES: Cc1nc2c(F)cc(-c3c[nH]c4ncc(C(=O)N5CCN(C)CC5)cc34)cc2n1C(C)C] | 493 |
| 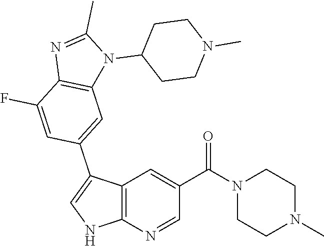 [SMILES: Cc1nc2c(F)cc(-c3c[nH]c4ncc(C(=O)N5CCN(C)CC5)cc34)cc2n1C1CCN(C)CC1] | 494 |
| 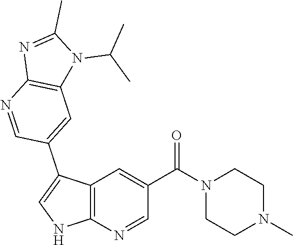 [SMILES: Cc1nc2ncc(-c3c[nH]c4ncc(C(=O)N5CCN(C)CC5)cc34)cc2n1C(C)C] | 495 |
| 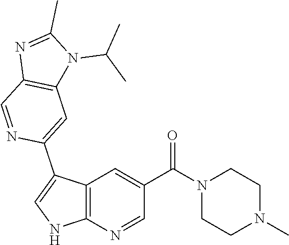 [SMILES: Cc1nc2cnc(-c3c[nH]c4ncc(C(=O)N5CCN(C)CC5)cc34)cc2n1C(C)C] | 496 |
| 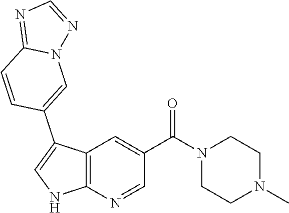 [SMILES: CN1CCN(C(=O)c2cnc3[nH]cc(-c4ccc5ncnn5c4)c3c2)CC1] | 497 |
| 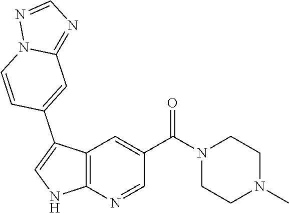 [SMILES: CN1CCN(C(=O)c2cnc3[nH]cc(-c4ccn5ncnc5c4)c3c2)CC1] | 498 |
| 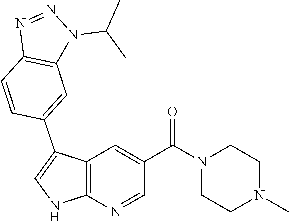 [SMILES: CC(C)n1nnc2ccc(-c3c[nH]c4ncc(C(=O)N5CCN(C)CC5)cc34)cc21] | 499 |
| 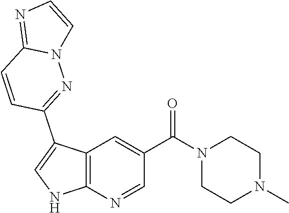 [SMILES: CN1CCN(C(=O)c2cnc3[nH]cc(-c4ccc5nccn5n4)c3c2)CC1] | 500 |
| 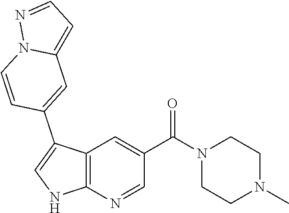 [SMILES: CN1CCN(C(=O)c2cnc3[nH]cc(-c4ccn5nccc5c4)c3c2)CC1] | 501 |
| 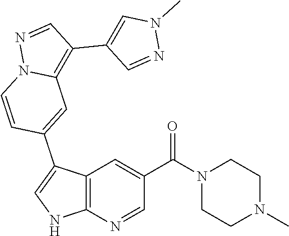 [SMILES: CN1CCN(C(=O)c2cnc3[nH]cc(-c4ccn5ncc(-c6cnn(C)c6)c5c4)c3c2)CC1] | 502 |
| 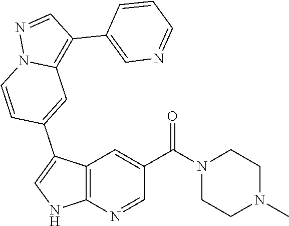 [SMILES: CN1CCN(C(=O)c2cnc3[nH]cc(-c4ccn5ncc(-c6cccnc6)c5c4)c3c2)CC1] | 503 |
| 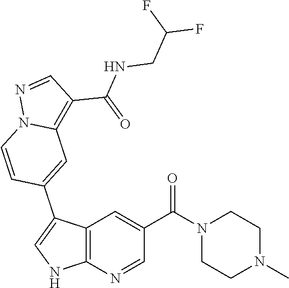 [SMILES: CN1CCN(C(=O)c2cnc3[nH]cc(-c4ccn5ncc(C(=O)NCC(F)F)c5c4)c3c2)CC1] | 504 |
| 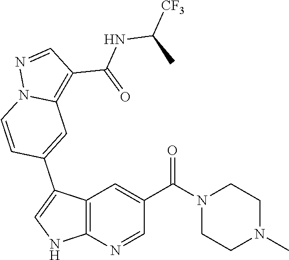 [SMILES: C[C@@H](NC(=O)c1cnn2ccc(-c3c[nH]c4ncc(C(=O)N5CCN(C)CC5)cc34)cc12)C(F)(F)F] | 505 |
| 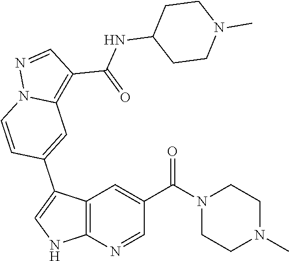 [SMILES: CN1CCC(NC(=O)c2cnn3ccc(-c4c[nH]c5ncc(C(=O)N6CCN(C)CC6)cc45)cc23)CC1] | 506 |
| 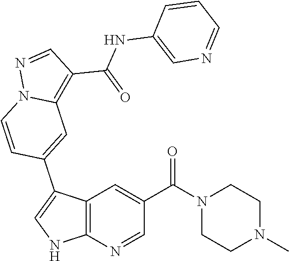 [SMILES: CN1CCN(C(=O)c2cnc3[nH]cc(-c4ccn5ncc(C(=O)Nc6cccnc6)c5c4)c3c2)CC1] | 507 |
| 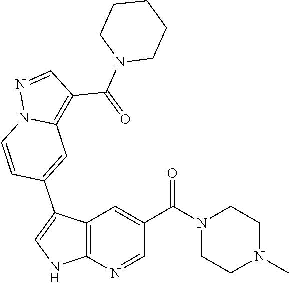 [SMILES: CN1CCN(C(=O)c2cnc3[nH]cc(-c4ccn5ncc(C(=O)N6CCCCC6)c5c4)c3c2)CC1] | 508 |
| 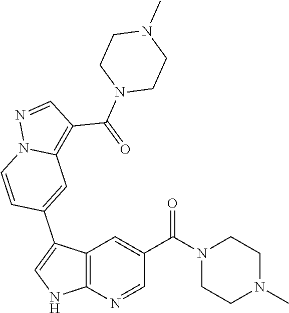 [SMILES: CN1CCN(C(=O)c2cnc3[nH]cc(-c4ccn5ncc(C(=O)N6CCN(C)CC6)c5c4)c3c2)CC1] | 509 |
| 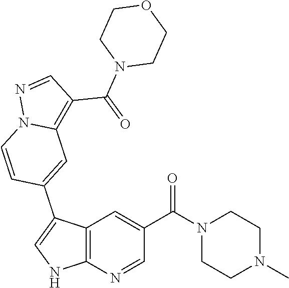 [SMILES: CN1CCN(C(=O)c2cnc3[nH]cc(-c4ccn5ncc(C(=O)N6CCOCC6)c5c4)c3c2)CC1] | 510 |
| 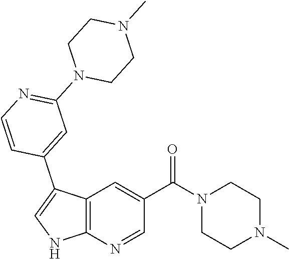 [SMILES: CN1CCN(C(=O)c2cnc3[nH]cc(-c4ccnc(N5CCN(C)CC5)c4)c3c2)CC1] | 511 |
| 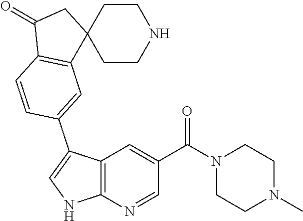 [SMILES: CN1CCN(C(=O)c2cnc3[nH]cc(-c4ccc5c(c4)C4(CCNCC4)CC5=O)c3c2)CC1] | 512 |
| 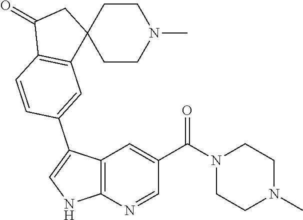 [SMILES: CN1CCN(C(=O)c2cnc3[nH]cc(-c4ccc5c(c4)C4(CCN(C)CC4)CC5=O)c3c2)CC1] | 513 |
| 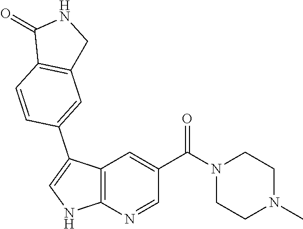 [SMILES: CN1CCN(C(=O)c2cnc3[nH]cc(-c4ccc5c(c4)CNC5=O)c3c2)CC1] | 514 |
| 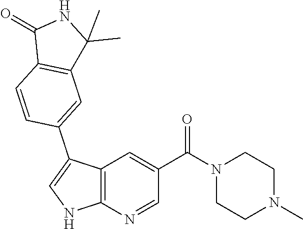 [SMILES: CN1CCN(C(=O)c2cnc3[nH]cc(-c4ccc5c(c4)C(C)(C)NC5=O)c3c2)CC1] | 515 |
| 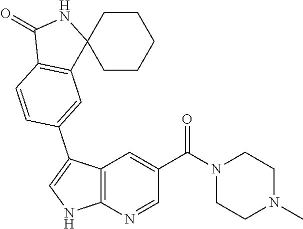 [SMILES: CN1CCN(C(=O)c2cnc3[nH]cc(-c4ccc5c(c4)C4(CCCCC4)NC5=O)c3c2)CC1] | 516 |
| 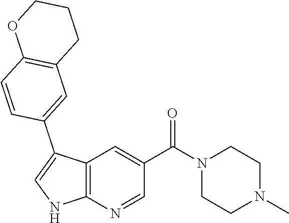 [SMILES: CN1CCN(C(=O)c2cnc3[nH]cc(-c4ccc5c(c4)CCCO5)c3c2)CC1] | 517 |
| 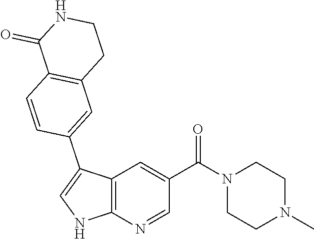 [SMILES: CN1CCN(C(=O)c2cnc3[nH]cc(-c4ccc5c(c4)CCNC5=O)c3c2)CC1] | 518 |
| 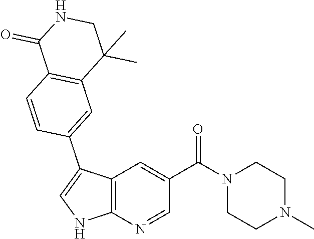 [SMILES: CN1CCN(C(=O)c2cnc3[nH]cc(-c4ccc5c(c4)C(C)(C)CNC5=O)c3c2)CC1] | 519 |
| 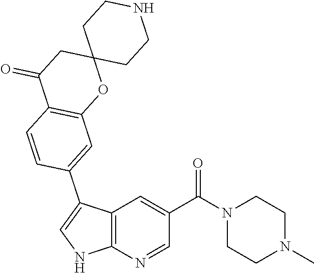 [SMILES: CN1CCN(C(=O)c2cnc3[nH]cc(-c4ccc5c(c4)OC4(CCNCC4)CC5=O)c3c2)CC1] | 520 |
| 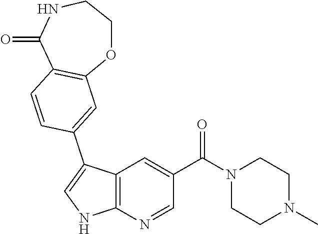 [SMILES: CN1CCN(C(=O)c2cnc3[nH]cc(-c4ccc5c(c4)OCCNC5=O)c3c2)CC1] | 521 |
| 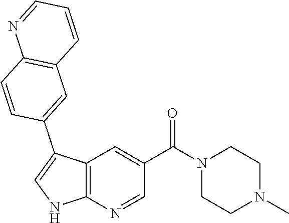 [SMILES: CN1CCN(C(=O)c2cnc3[nH]cc(-c4ccc5ncccc5c4)c3c2)CC1] | 522 |
| 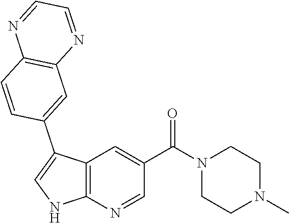 [SMILES: CN1CCN(C(=O)c2cnc3[nH]cc(-c4ccc5nccnc5c4)c3c2)CC1] | 523 |
| 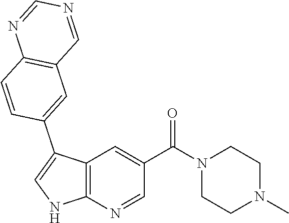 [SMILES: CN1CCN(C(=O)c2cnc3[nH]cc(-c4ccc5ncncc5c4)c3c2)CC1] | 524 |
| 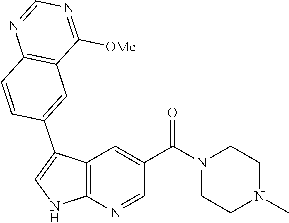 [SMILES: COc1ncnc2ccc(-c3c[nH]c4ncc(C(=O)N5CCN(C)CC5)cc34)cc12] | 525 |
| 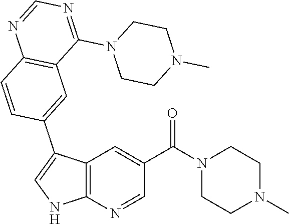 [SMILES: CN1CCN(C(=O)c2cnc3[nH]cc(-c4ccc5ncnc(N6CCN(C)CC6)c5c4)c3c2)CC1] | 526 |
| 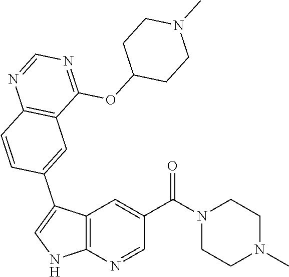 [SMILES: CN1CCC(Oc2ncnc3ccc(-c4c[nH]c5ncc(C(=O)N6CCN(C)CC6)cc45)cc23)CC1] | 527 |
| 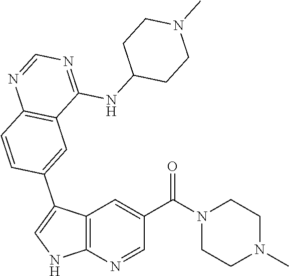 [SMILES: CN1CCC(Nc2ncnc3ccc(-c4c[nH]c5ncc(C(=O)N6CCN(C)CC6)cc45)cc23)CC1] | 528 |
| 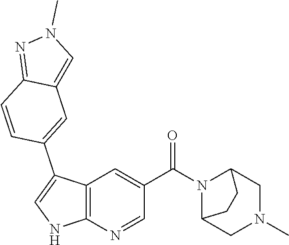 [SMILES: CN1CC2CCC(C1)N2C(=O)c1cnc2[nH]cc(-c3ccc4nn(C)cc4c3)c2c1] | 529 |
| 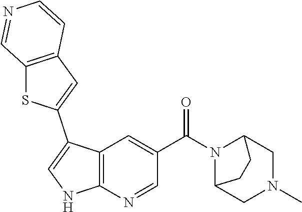 [SMILES: CN1CC2CCC(C1)N2C(=O)c1cnc2[nH]cc(-c3cc4ccncc4s3)c2c1] | 530 |
| 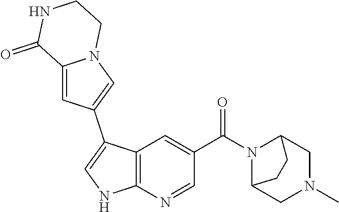 [SMILES: CN1CC2CCC(C1)N2C(=O)c1cnc2[nH]cc(-c3cc4n(c3)CCNC4=O)c2c1] | 531 |
| 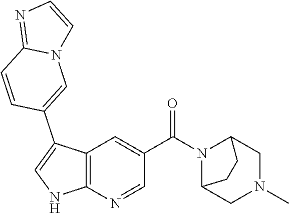 [SMILES: CN1CC2CCC(C1)N2C(=O)c1cnc2[nH]cc(-c3ccc4nccn4c3)c2c1] | 532 |
| 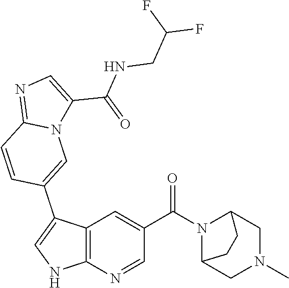 [SMILES: CN1CC2CCC(C1)N2C(=O)c1cnc2[nH]cc(-c3ccc4ncc(C(=O)NCC(F)F)n4c3)c2c1] | 533 |
| 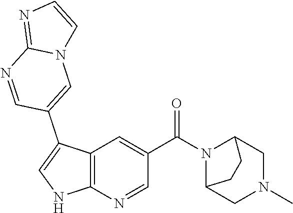 [SMILES: CN1CC2CCC(C1)N2C(=O)c1cnc2[nH]cc(-c3cnc4nccn4c3)c2c1] | 534 |
| 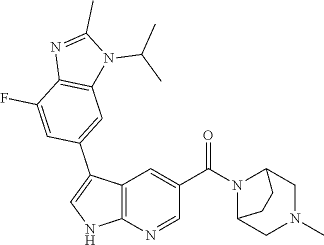 [SMILES: Cc1nc2c(F)cc(-c3c[nH]c4ncc(C(=O)N5C6CCC5CN(C)C6)cc34)cc2n1C(C)C] | 535 |
| 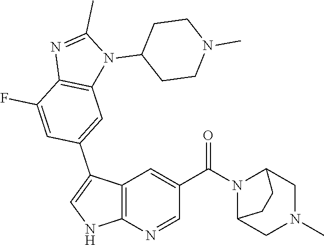 [SMILES: Cc1nc2c(F)cc(-c3c[nH]c4ncc(C(=O)N5C6CCC5CN(C)C6)cc34)cc2n1C1CCN(C)CC1] | 536 |
| 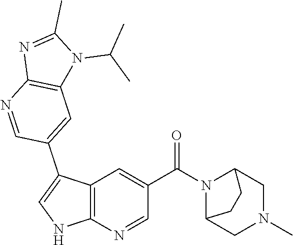 [SMILES: Cc1nc2ncc(-c3c[nH]c4ncc(C(=O)N5C6CCC5CN(C)C6)cc34)cc2n1C(C)C] | 537 |
| 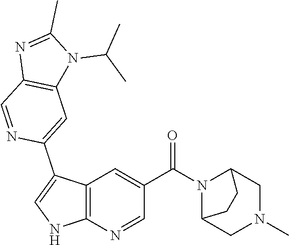 [SMILES: Cc1nc2cnc(-c3c[nH]c4ncc(C(=O)N5C6CCC5CN(C)C6)cc34)cc2n1C(C)C] | 538 |
| 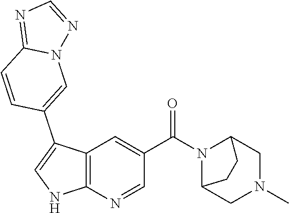 [SMILES: CN1CC2CCC(C1)N2C(=O)c1cnc2[nH]cc(-c3ccc4ncnn4c3)c2c1] | 539 |
| 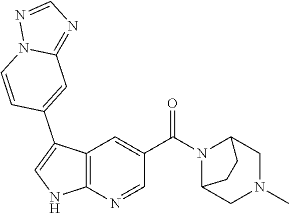 [SMILES: CN1CC2CCC(C1)N2C(=O)c1cnc2[nH]cc(-c3ccn4ncnc4c3)c2c1] | 540 |
| 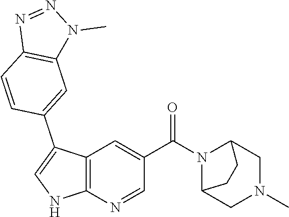 [SMILES: CN1CC2CCC(C1)N2C(=O)c1cnc2[nH]cc(-c3ccc4nnn(C)c4c3)c2c1] | 541 |
| 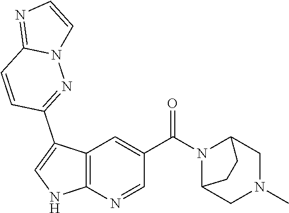 [SMILES: CN1CC2CCC(C1)N2C(=O)c1cnc2[nH]cc(-c3ccc4nccn4n3)c2c1] | 542 |
| 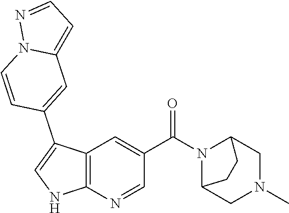 [SMILES: CN1CC2CCC(C1)N2C(=O)c1cnc2[nH]cc(-c3ccn4nccc4c3)c2c1] | 543 |
| 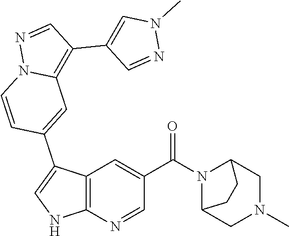 [SMILES: CN1CC2CCC(C1)N2C(=O)c1cnc2[nH]cc(-c3ccn4ncc(-c5cnn(C)c5)c4c3)c2c1] | 544 |
| 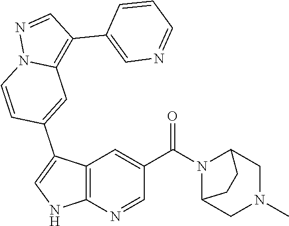 [SMILES: CN1CC2CCC(C1)N2C(=O)c1cnc2[nH]cc(-c3ccn4ncc(-c5cccnc5)c4c3)c2c1] | 545 |
| 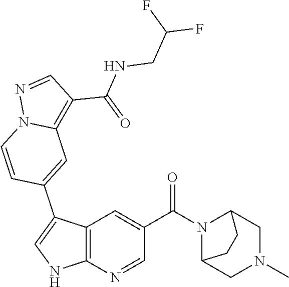 [SMILES: CN1CC2CCC(C1)N2C(=O)c1cnc2[nH]cc(-c3ccn4ncc(C(=O)NCC(F)F)c4c3)c2c1] | 546 |
| 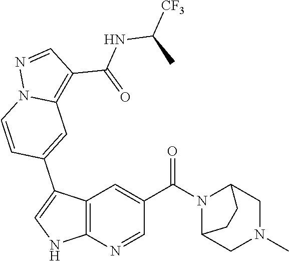 [SMILES: C[C@@H](NC(=O)c1cnn2ccc(-c3c[nH]c4ncc(C(=O)N5C6CCC5CN(C)C6)cc34)cc12)C(F)(F)F] | 547 |
| 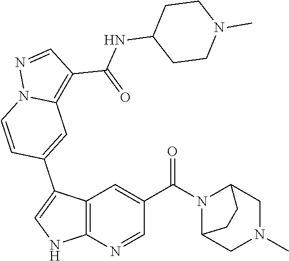 [SMILES: CN1CCC(NC(=O)c2cnn3ccc(-c4c[nH]c5ncc(C(=O)N6C7CCC6CN(C)C7)cc45)cc23)CC1] | 548 |
| 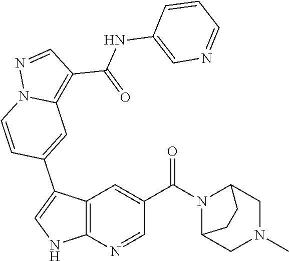 [SMILES: CN1CC2CCC(C1)N2C(=O)c1cnc2[nH]cc(-c3ccn4ncc(C(=O)Nc5cccnc5)c4c3)c2c1] | 549 |
| 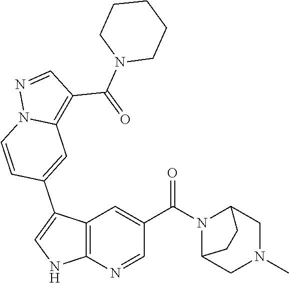 [SMILES: CN1CC2CCC(C1)N2C(=O)c1cnc2[nH]cc(-c3ccn4ncc(C(=O)N5CCCCC5)c4c3)c2c1] | 550 |
| 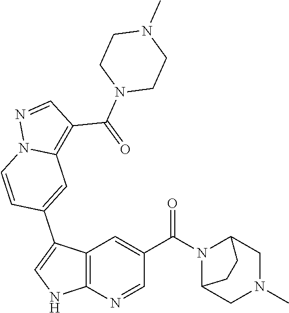 [SMILES: CN1CCN(C(=O)c2cnn3ccc(-c4c[nH]c5ncc(C(=O)N6C7CCC6CN(C)C7)cc45)cc23)CC1] | 551 |
| 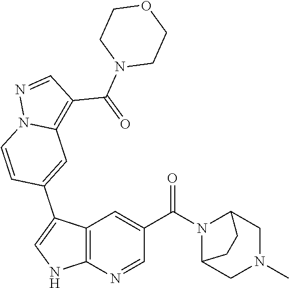 [SMILES: CN1CC2CCC(C1)N2C(=O)c1cnc2[nH]cc(-c3ccn4ncc(C(=O)N5CCOCC5)c4c3)c2c1] | 552 |
| 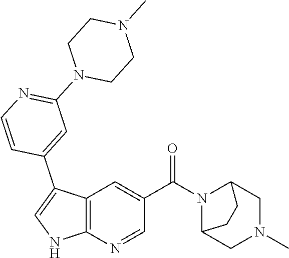 [SMILES: CN1CCN(c2cc(-c3c[nH]c4ncc(C(=O)N5C6CCC5CN(C)C6)cc34)ccn2)CC1] | 553 |
| 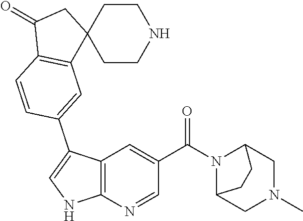 [SMILES: CN1CC2CCC(C1)N2C(=O)c1cnc2[nH]cc(-c3ccc4c(c3)C3(CCNCC3)CC4=O)c2c1] | 554 |
| 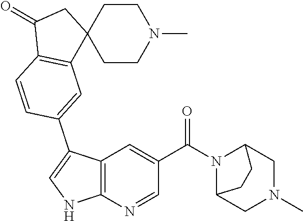 [SMILES: CN1CCC2(CC1)CC(=O)c1ccc(-c3c[nH]c4ncc(C(=O)N5C6CCC5CN(C)C6)cc34)cc12] | 555 |
| 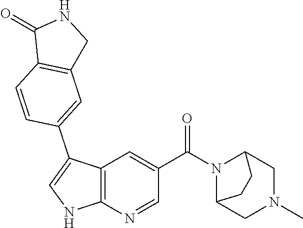 [SMILES: CN1CC2CCC(C1)N2C(=O)c1cnc2[nH]cc(-c3ccc4c(c3)CNC4=O)c2c1] | 556 |
| 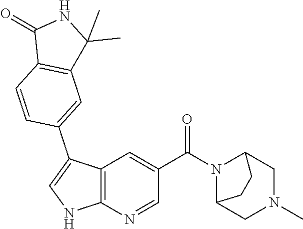 [SMILES: CN1CC2CCC(C1)N2C(=O)c1cnc2[nH]cc(-c3ccc4c(c3)C(C)(C)NC4=O)c2c1] | 557 |
| 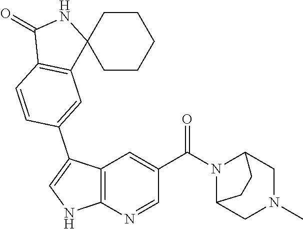 [SMILES: CN1CC2CCC(C1)N2C(=O)c1cnc2[nH]cc(-c3ccc4c(c3)C3(CCCCC3)NC4=O)c2c1] | 558 |
| 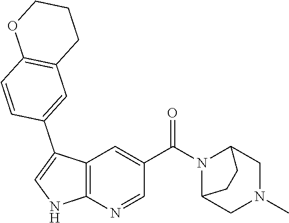 [SMILES: CN1CC2CCC(C1)N2C(=O)c1cnc2[nH]cc(-c3ccc4c(c3)CCCO4)c2c1] | 559 |
| 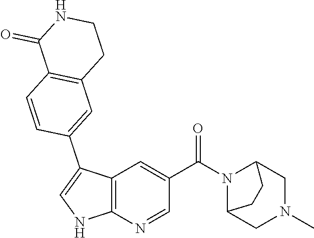 [SMILES: CN1CC2CCC(C1)N2C(=O)c1cnc2[nH]cc(-c3ccc4c(c3)CCNC4=O)c2c1] | 560 |
| 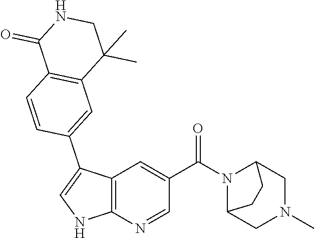 [SMILES: CN1CC2CCC(C1)N2C(=O)c1cnc2[nH]cc(-c3ccc4c(c3)C(C)(C)CNC4=O)c2c1] | 561 |
| 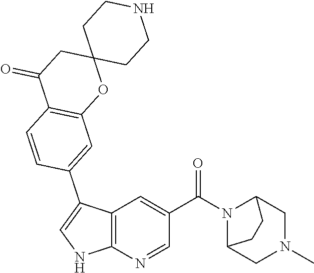 [SMILES: CN1CC2CCC(C1)N2C(=O)c1cnc2[nH]cc(-c3ccc4c(c3)OC3(CCNCC3)CC4=O)c2c1] | 562 |
| 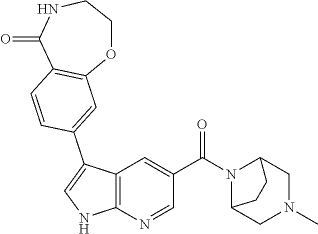 [SMILES: CN1CC2CCC(C1)N2C(=O)c1cnc2[nH]cc(-c3ccc4c(c3)OCCNC4=O)c2c1] | 563 |
| 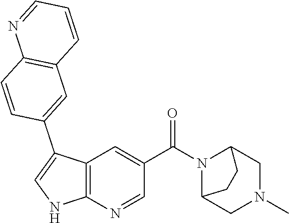 [SMILES: CN1CC2CCC(C1)N2C(=O)c1cnc2[nH]cc(-c3ccc4ncccc4c3)c2c1] | 564 |
| 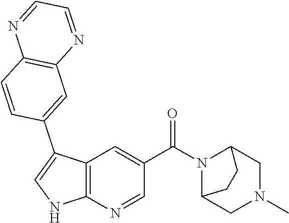 [SMILES: CN1CC2CCC(C1)N2C(=O)c1cnc2[nH]cc(-c3ccc4nccnc4c3)c2c1] | 565 |
| 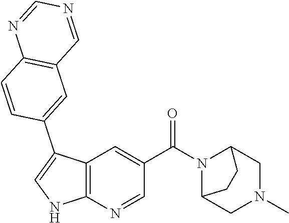 [SMILES: CN1CC2CCC(C1)N2C(=O)c1cnc2[nH]cc(-c3ccc4ncncc4c3)c2c1] | 566 |
| 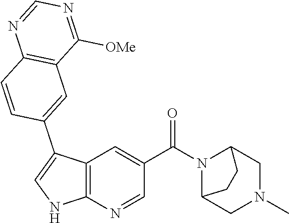 [SMILES: COc1ncnc2ccc(-c3c[nH]c4ncc(C(=O)N5C6CCC5CN(C)C6)cc34)cc12] | 567 |
| 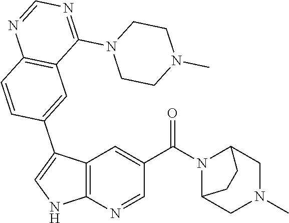 [SMILES: CN1CCN(c2ncnc3ccc(-c4c[nH]c5ncc(C(=O)N6C7CCC6CN(C)C7)cc45)cc23)CC1] | 568 |
| 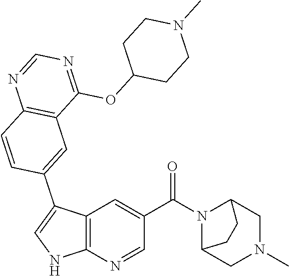 [SMILES: CN1CCC(Oc2ncnc3ccc(-c4c[nH]c5ncc(C(=O)N6C7CCC6CN(C)C7)cc45)cc23)CC1] | 569 |
| 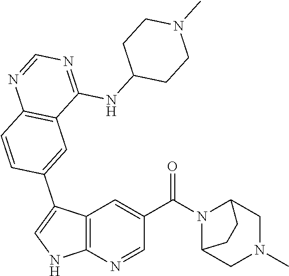 [SMILES: CN1CCC(Nc2ncnc3ccc(-c4c[nH]c5ncc(C(=O)N6C7CCC6CN(C)C7)cc45)cc23)CC1] | 570 |
| 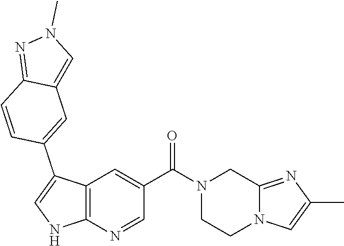 [SMILES: Cc1cn2c(n1)CN(C(=O)c1cnc3[nH]cc(-c4ccc5nn(C)cc5c4)c3c1)CC2] | 571 |
| 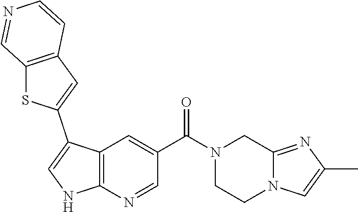 [SMILES: Cc1cn2c(n1)CN(C(=O)c1cnc3[nH]cc(-c4cc5ccncc5s4)c3c1)CC2] | 572 |
| 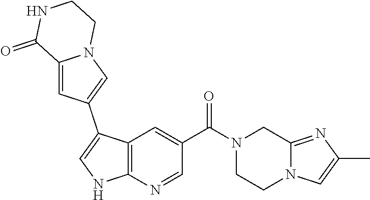 [SMILES: Cc1cn2c(n1)CN(C(=O)c1cnc3[nH]cc(-c4cc5n(c4)CCNC5=O)c3c1)CC2] | 573 |
| 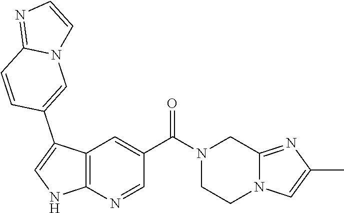 [SMILES: Cc1cn2c(n1)CN(C(=O)c1cnc3[nH]cc(-c4ccc5nccn5c4)c3c1)CC2] | 574 |
| 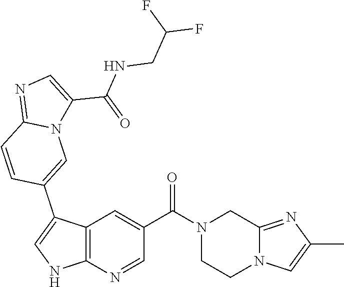 [SMILES: Cc1cn2c(n1)CN(C(=O)c1cnc3[nH]cc(-c4ccc5ncc(C(=O)NCC(F)F)n5c4)c3c1)CC2] | 575 |
| 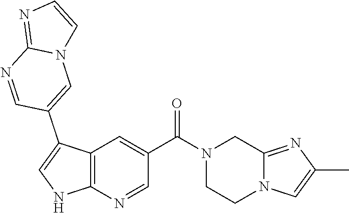 [SMILES: Cc1cn2c(n1)CN(C(=O)c1cnc3[nH]cc(-c4cnc5nccn5c4)c3c1)CC2] | 576 |
| 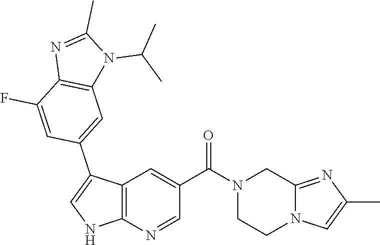 [SMILES: Cc1cn2c(n1)CN(C(=O)c1cnc3[nH]cc(-c4cc(F)c5nc(C)n(C(C)C)c5c4)c3c1)CC2] | 577 |
| 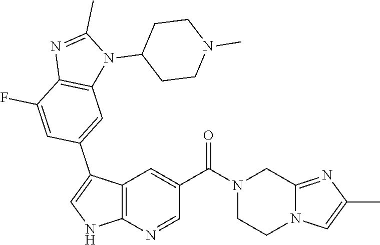 [SMILES: Cc1cn2c(n1)CN(C(=O)c1cnc3[nH]cc(-c4cc(F)c5nc(C)n(C6CCN(C)CC6)c5c4)c3c1)CC2] | 578 |
| 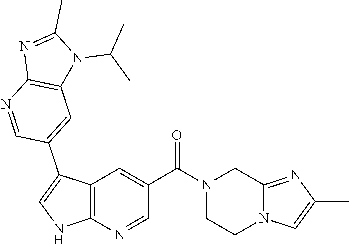 [SMILES: Cc1cn2c(n1)CN(C(=O)c1cnc3[nH]cc(-c4cnc5nc(C)n(C(C)C)c5c4)c3c1)CC2] | 579 |
| 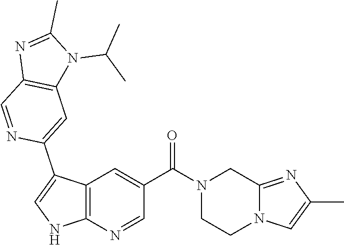 [SMILES: Cc1cn2c(n1)CN(C(=O)c1cnc3[nH]cc(-c4cc5c(cn4)nc(C)n5C(C)C)c3c1)CC2] | 580 |
| 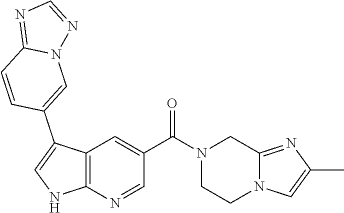 [SMILES: Cc1cn2c(n1)CN(C(=O)c1cnc3[nH]cc(-c4ccc5ncnn5c4)c3c1)CC2] | 581 |
| 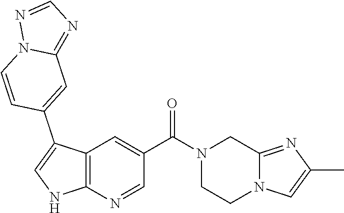 [SMILES: Cc1cn2c(n1)CN(C(=O)c1cnc3[nH]cc(-c4ccn5ncnc5c4)c3c1)CC2] | 582 |
| 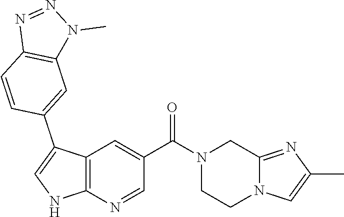 [SMILES: Cc1cn2c(n1)CN(C(=O)c1cnc3[nH]cc(-c4ccc5nnn(C)c5c4)c3c1)CC2] | 583 |
| 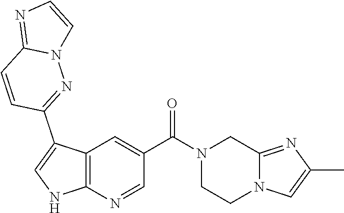 [SMILES: Cc1cn2c(n1)CN(C(=O)c1cnc3[nH]cc(-c4ccc5nccn5n4)c3c1)CC2] | 584 |
| 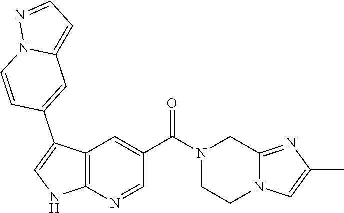 [SMILES: Cc1cn2c(n1)CN(C(=O)c1cnc3[nH]cc(-c4ccn5nccc5c4)c3c1)CC2] | 585 |
| 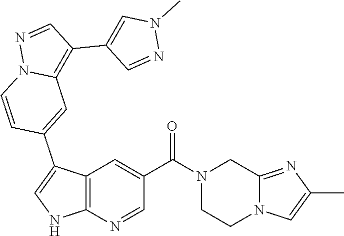 [SMILES: Cc1cn2c(n1)CN(C(=O)c1cnc3[nH]cc(-c4ccn5ncc(-c6cnn(C)c6)c5c4)c3c1)CC2] | 586 |
| 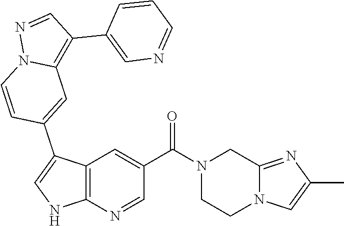 [SMILES: Cc1cn2c(n1)CN(C(=O)c1cnc3[nH]cc(-c4ccn5ncc(-c6cccnc6)c5c4)c3c1)CC2] | 587 |
| 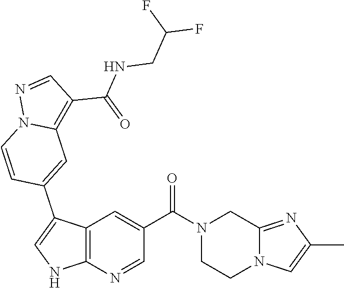 [SMILES: Cc1cn2c(n1)CN(C(=O)c1cnc3[nH]cc(-c4ccn5ncc(C(=O)NCC(F)F)c5c4)c3c1)CC2] | 588 |
| 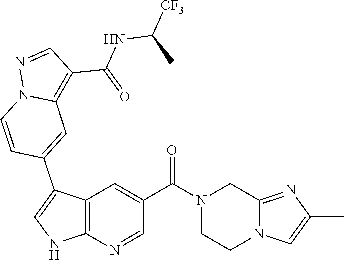 [SMILES: Cc1cn2c(n1)CN(C(=O)c1cnc3[nH]cc(-c4ccn5ncc(C(=O)N[C@H](C)C(F)(F)F)c5c4)c3c1)CC2] | 589 |
| 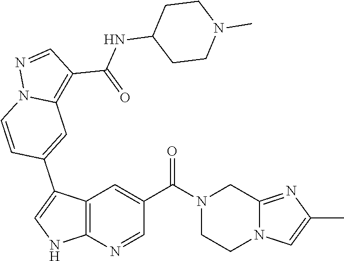 [SMILES: Cc1cn2c(n1)CN(C(=O)c1cnc3[nH]cc(-c4ccn5ncc(C(=O)NC6CCN(C)CC6)c5c4)c3c1)CC2] | 590 |
| 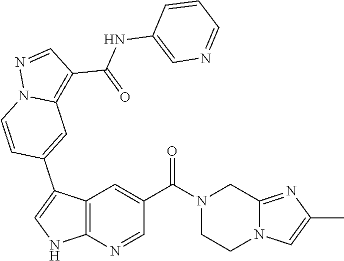 [SMILES: Cc1cn2c(n1)CN(C(=O)c1cnc3[nH]cc(-c4ccn5ncc(C(=O)Nc6cccnc6)c5c4)c3c1)CC2] | 591 |
| 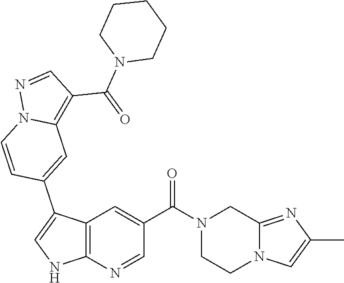 [SMILES: Cc1cn2c(n1)CN(C(=O)c1cnc3[nH]cc(-c4ccn5ncc(C(=O)N6CCCCC6)c5c4)c3c1)CC2] | 592 |
| 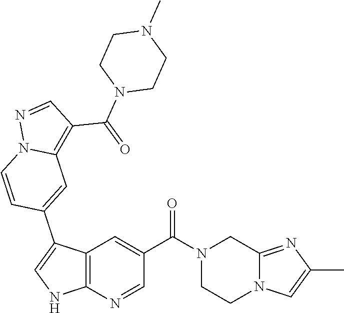 [SMILES: Cc1cn2c(n1)CN(C(=O)c1cnc3[nH]cc(-c4ccn5ncc(C(=O)N6CCN(C)CC6)c5c4)c3c1)CC2] | 593 |
| 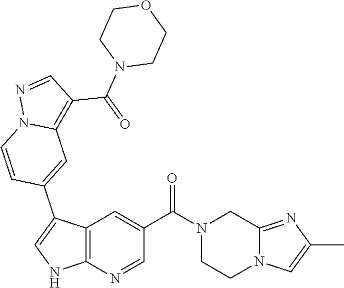 [SMILES: Cc1cn2c(n1)CN(C(=O)c1cnc3[nH]cc(-c4ccn5ncc(C(=O)N6CCOCC6)c5c4)c3c1)CC2] | 594 |
| 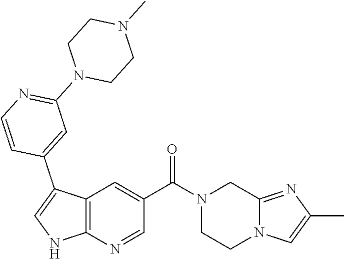 [SMILES: Cc1cn2c(n1)CN(C(=O)c1cnc3[nH]cc(-c4ccnc(N5CCN(C)CC5)c4)c3c1)CC2] | 595 |
| 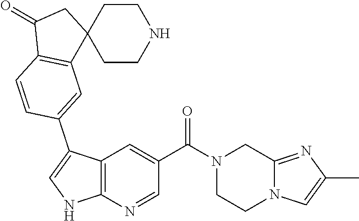 [SMILES: Cc1cn2c(n1)CN(C(=O)c1cnc3[nH]cc(-c4ccc5c(c4)C4(CCNCC4)CC5=O)c3c1)CC2] | 596 |
| 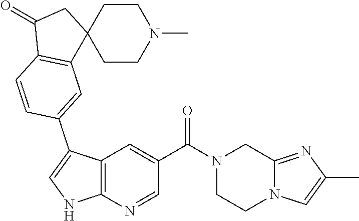 [SMILES: Cc1cn2c(n1)CN(C(=O)c1cnc3[nH]cc(-c4ccc5c(c4)C4(CCN(C)CC4)CC5=O)c3c1)CC2] | 597 |
| 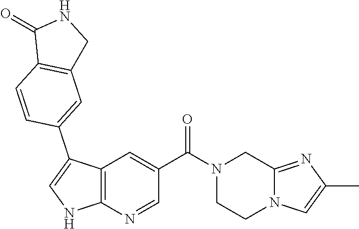 [SMILES: Cc1cn2c(n1)CN(C(=O)c1cnc3[nH]cc(-c4ccc5c(c4)CNC5=O)c3c1)CC2] | 598 |
| 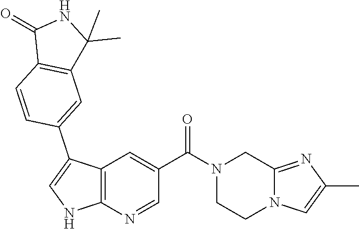 [SMILES: Cc1cn2c(n1)CN(C(=O)c1cnc3[nH]cc(-c4ccc5c(c4)C(C)(C)NC5=O)c3c1)CC2] | 599 |
| 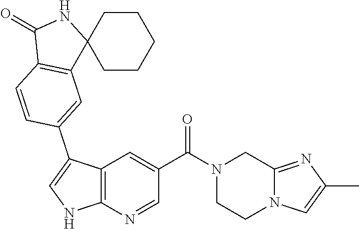 [SMILES: Cc1cn2c(n1)CN(C(=O)c1cnc3[nH]cc(-c4ccc5c(c4)C4(CCCCC4)NC5=O)c3c1)CC2] | 600 |
| 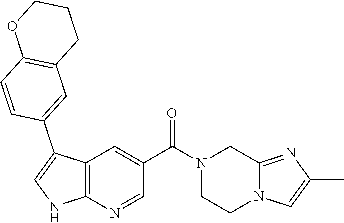 [SMILES: Cc1cn2c(n1)CN(C(=O)c1cnc3[nH]cc(-c4ccc5c(c4)CCCO5)c3c1)CC2] | 601 |
| 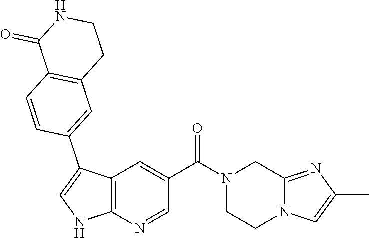 [SMILES: Cc1cn2c(n1)CN(C(=O)c1cnc3[nH]cc(-c4ccc5c(c4)CCNC5=O)c3c1)CC2] | 602 |
| 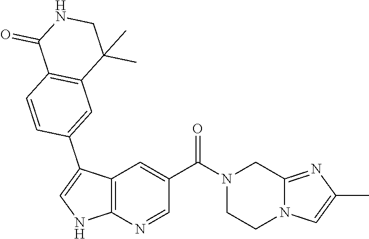 [SMILES: Cc1cn2c(n1)CN(C(=O)c1cnc3[nH]cc(-c4ccc5c(c4)C(C)(C)CNC5=O)c3c1)CC2] | 603 |
| 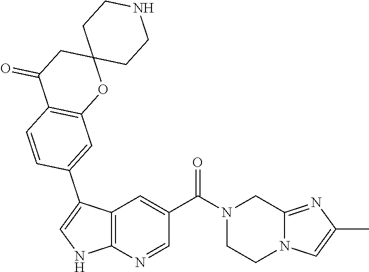 [SMILES: Cc1cn2c(n1)CN(C(=O)c1cnc3[nH]cc(-c4ccc5c(c4)OC4(CCNCC4)CC5=O)c3c1)CC2] | 604 |
| 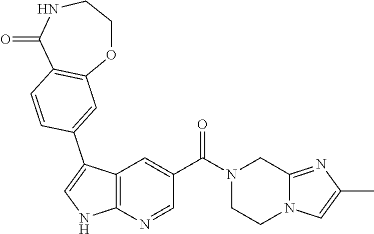 [SMILES: Cc1cn2c(n1)CN(C(=O)c1cnc3[nH]cc(-c4ccc5c(c4)OCCNC5=O)c3c1)CC2] | 605 |
| 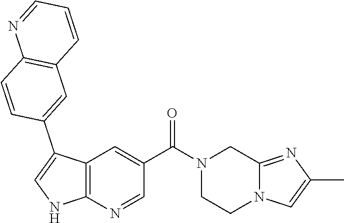 [SMILES: Cc1cn2c(n1)CN(C(=O)c1cnc3[nH]cc(-c4ccc5ncccc5c4)c3c1)CC2] | 606 |
| 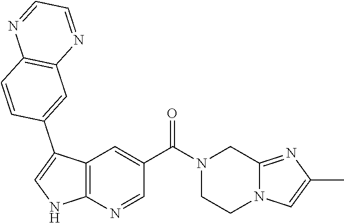 [SMILES: Cc1cn2c(n1)CN(C(=O)c1cnc3[nH]cc(-c4ccc5nccnc5c4)c3c1)CC2] | 607 |
| 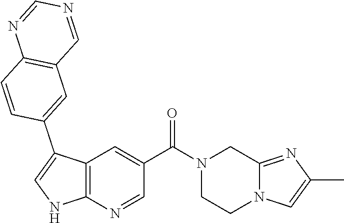 [SMILES: Cc1cn2c(n1)CN(C(=O)c1cnc3[nH]cc(-c4ccc5ncncc5c4)c3c1)CC2] | 608 |
| 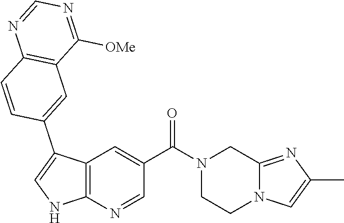 [SMILES: COc1ncnc2ccc(-c3c[nH]c4ncc(C(=O)N5CCn6cc(C)nc6C5)cc34)cc12] | 609 |
| 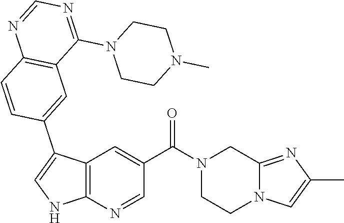 [SMILES: Cc1cn2c(n1)CN(C(=O)c1cnc3[nH]cc(-c4ccc5ncnc(N6CCN(C)CC6)c5c4)c3c1)CC2] | 610 |
| 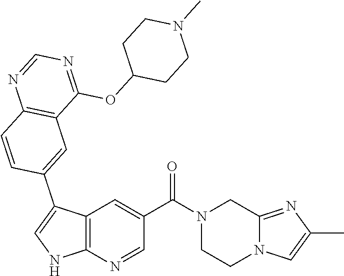 [SMILES: Cc1cn2c(n1)CN(C(=O)c1cnc3[nH]cc(-c4ccc5ncnc(OC6CCN(C)CC6)c5c4)c3c1)CC2] | 611 |
| 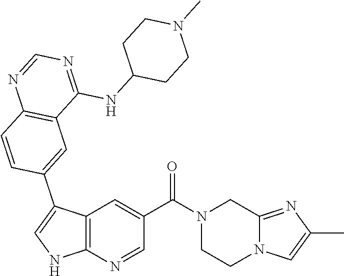 [SMILES: Cc1cn2c(n1)CN(C(=O)c1cnc3[nH]cc(-c4ccc5ncnc(NC6CCN(C)CC6)c5c4)c3c1)CC2] | 612 |
| 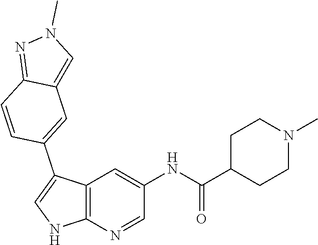 [SMILES: CN1CCC(C(=O)Nc2cnc3[nH]cc(-c4ccc5nn(C)cc5c4)c3c2)CC1] | 613 |
| 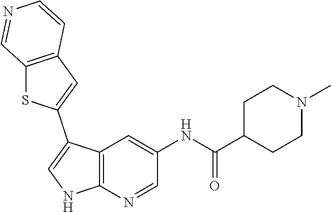 [SMILES: CN1CCC(C(=O)Nc2cnc3[nH]cc(-c4cc5ccncc5s4)c3c2)CC1] | 614 |
| 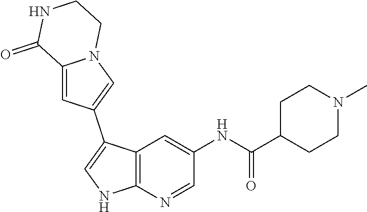 [SMILES: CN1CCC(C(=O)Nc2cnc3[nH]cc(-c4cc5n(c4)CCNC5=O)c3c2)CC1] | 615 |
| 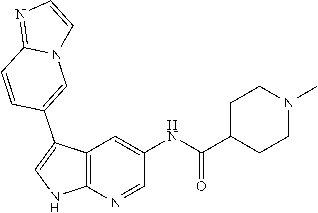 [SMILES: CN1CCC(C(=O)Nc2cnc3[nH]cc(-c4ccc5nccn5c4)c3c2)CC1] | 616 |
| 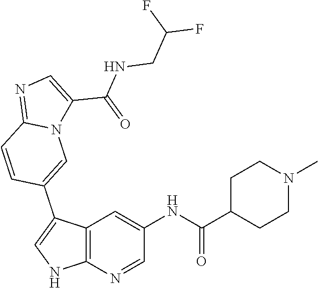 [SMILES: CN1CCC(C(=O)Nc2cnc3[nH]cc(-c4ccc5ncc(C(=O)NCC(F)F)n5c4)c3c2)CC1] | 617 |
| 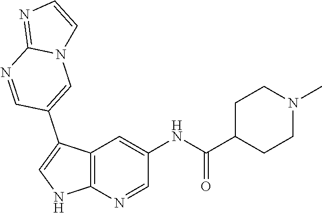 [SMILES: CN1CCC(C(=O)Nc2cnc3[nH]cc(-c4cnc5nccn5c4)c3c2)CC1] | 618 |
| 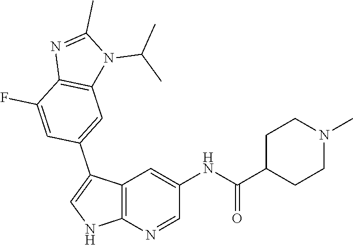 [SMILES: Cc1nc2c(F)cc(-c3c[nH]c4ncc(NC(=O)C5CCN(C)CC5)cc34)cc2n1C(C)C] | 619 |
| 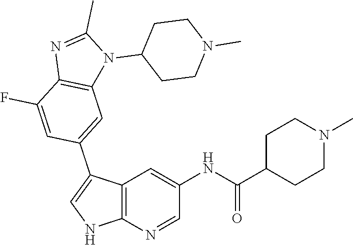 [SMILES: Cc1nc2c(F)cc(-c3c[nH]c4ncc(NC(=O)C5CCN(C)CC5)cc34)cc2n1C1CCN(C)CC1] | 620 |
| 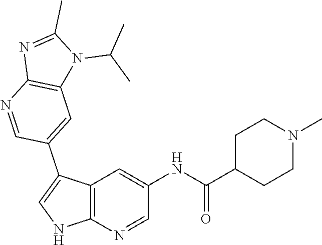 [SMILES: Cc1nc2ncc(-c3c[nH]c4ncc(NC(=O)C5CCN(C)CC5)cc34)cc2n1C(C)C] | 621 |
| 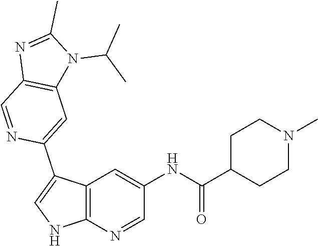 [SMILES: Cc1nc2cnc(-c3c[nH]c4ncc(NC(=O)C5CCN(C)CC5)cc34)cc2n1C(C)C] | 622 |
| 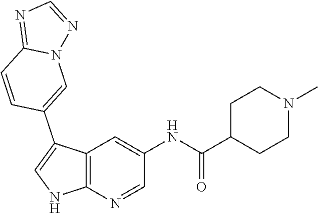 [SMILES: CN1CCC(C(=O)Nc2cnc3[nH]cc(-c4ccc5ncnn5c4)c3c2)CC1] | 623 |
| 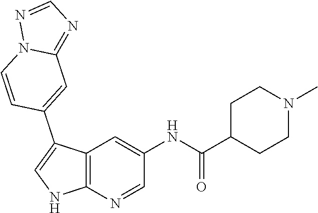 [SMILES: CN1CCC(C(=O)Nc2cnc3[nH]cc(-c4ccn5ncnc5c4)c3c2)CC1] | 624 |
| 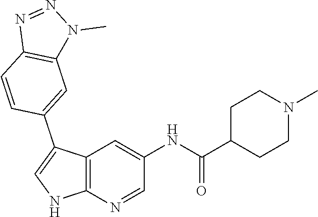 [SMILES: CN1CCC(C(=O)Nc2cnc3[nH]cc(-c4ccc5nnn(C)c5c4)c3c2)CC1] | 625 |
| 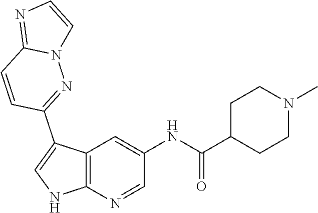 [SMILES: CN1CCC(C(=O)Nc2cnc3[nH]cc(-c4ccc5nccn5n4)c3c2)CC1] | 626 |
| 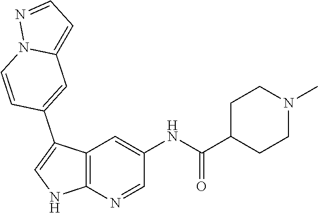 [SMILES: CN1CCC(C(=O)Nc2cnc3[nH]cc(-c4ccn5nccc5c4)c3c2)CC1] | 627 |
| 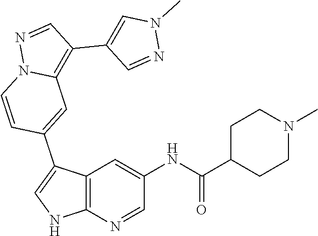 [SMILES: CN1CCC(C(=O)Nc2cnc3[nH]cc(-c4ccn5ncc(-c6cnn(C)c6)c5c4)c3c2)CC1] | 628 |
| 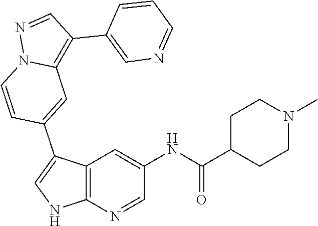 [SMILES: CN1CCC(C(=O)Nc2cnc3[nH]cc(-c4ccn5ncc(-c6cccnc6)c5c4)c3c2)CC1] | 629 |
| 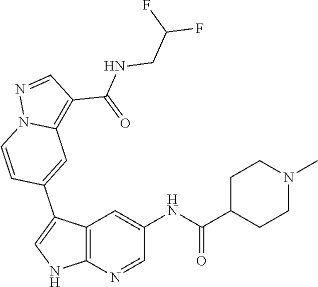 [SMILES: CN1CCC(C(=O)Nc2cnc3[nH]cc(-c4ccn5ncc(C(=O)NCC(F)F)c5c4)c3c2)CC1] | 630 |
| 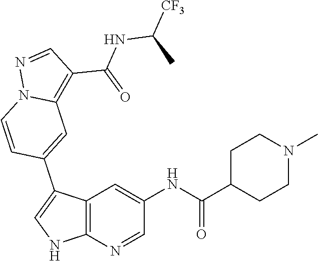 [SMILES: C[C@@H](NC(=O)c1cnn2ccc(-c3c[nH]c4ncc(NC(=O)C5CCN(C)CC5)cc34)cc12)C(F)(F)F] | 631 |
| 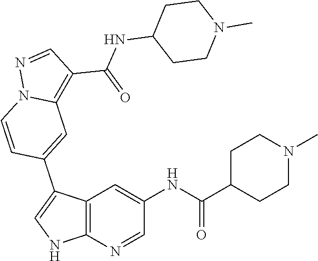 [SMILES: CN1CCC(NC(=O)c2cnn3ccc(-c4c[nH]c5ncc(NC(=O)C6CCN(C)CC6)cc45)cc23)CC1] | 632 |
| 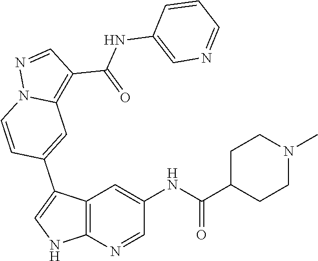 [SMILES: CN1CCC(C(=O)Nc2cnc3[nH]cc(-c4ccn5ncc(C(=O)Nc6cccnc6)c5c4)c3c2)CC1] | 633 |
| 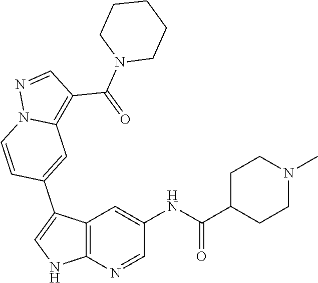 [SMILES: CN1CCC(C(=O)Nc2cnc3[nH]cc(-c4ccn5ncc(C(=O)N6CCCCC6)c5c4)c3c2)CC1] | 634 |
| 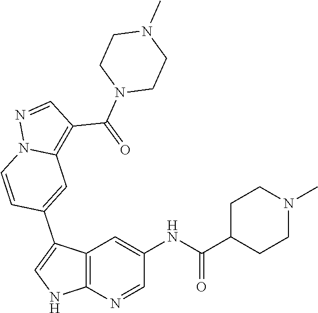 [SMILES: CN1CCC(C(=O)Nc2cnc3[nH]cc(-c4ccn5ncc(C(=O)N6CCN(C)CC6)c5c4)c3c2)CC1] | 635 |
| 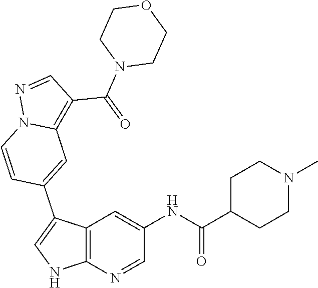 [SMILES: CN1CCC(C(=O)Nc2cnc3[nH]cc(-c4ccn5ncc(C(=O)N6CCOCC6)c5c4)c3c2)CC1] | 636 |
| 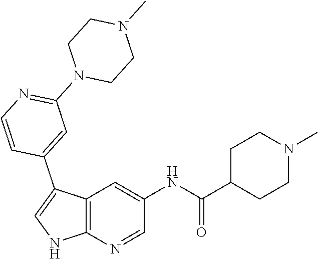 [SMILES: CN1CCC(C(=O)Nc2cnc3[nH]cc(-c4ccnc(N5CCN(C)CC5)c4)c3c2)CC1] | 637 |
| 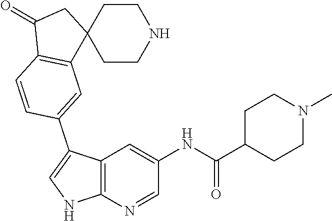 [SMILES: CN1CCC(C(=O)Nc2cnc3[nH]cc(-c4ccc5c(c4)C4(CCNCC4)CC5=O)c3c2)CC1] | 638 |
| 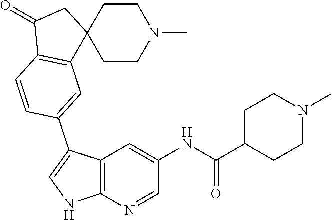 [SMILES: CN1CCC(C(=O)Nc2cnc3[nH]cc(-c4ccc5c(c4)C4(CCN(C)CC4)CC5=O)c3c2)CC1] | 639 |
| 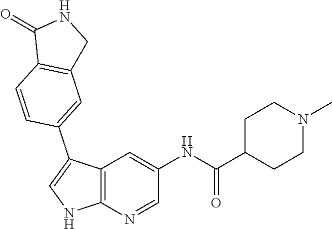 [SMILES: CN1CCC(C(=O)Nc2cnc3[nH]cc(-c4ccc5c(c4)CNC5=O)c3c2)CC1] | 640 |
| 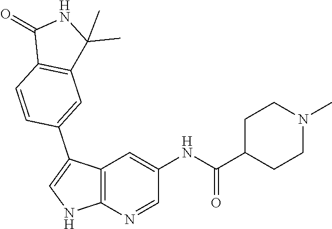 [SMILES: CN1CCC(C(=O)Nc2cnc3[nH]cc(-c4ccc5c(c4)C(C)(C)NC5=O)c3c2)CC1] | 641 |
| 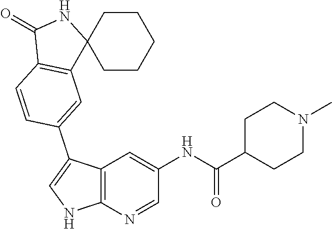 [SMILES: CN1CCC(C(=O)Nc2cnc3[nH]cc(-c4ccc5c(c4)C4(CCCCC4)NC5=O)c3c2)CC1] | 642 |
| 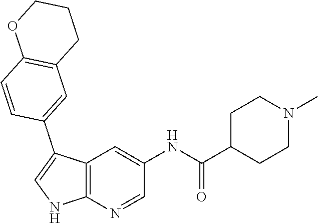 [SMILES: CN1CCC(C(=O)Nc2cnc3[nH]cc(-c4ccc5c(c4)CCCO5)c3c2)CC1] | 643 |
| 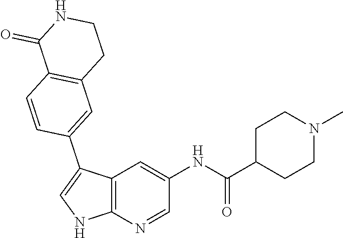 [SMILES: CN1CCC(C(=O)Nc2cnc3[nH]cc(-c4ccc5c(c4)CCNC5=O)c3c2)CC1] | 644 |
| 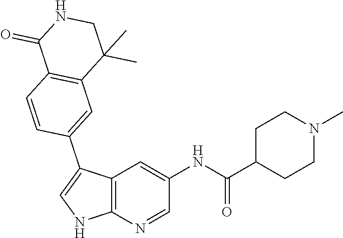 [SMILES: CN1CCC(C(=O)Nc2cnc3[nH]cc(-c4ccc5c(c4)C(C)(C)CNC5=O)c3c2)CC1] | 645 |
| 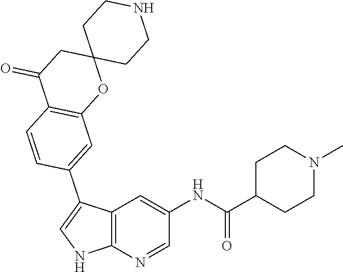 [SMILES: CN1CCC(C(=O)Nc2cnc3[nH]cc(-c4ccc5c(c4)OC4(CCNCC4)CC5=O)c3c2)CC1] | 646 |
| 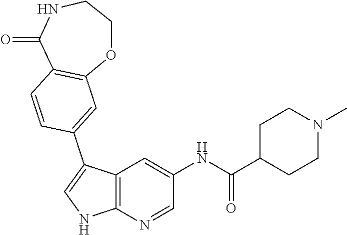 [SMILES: CN1CCC(C(=O)Nc2cnc3[nH]cc(-c4ccc5c(c4)OCCNC5=O)c3c2)CC1] | 647 |
| 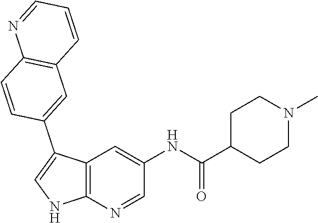 [SMILES: CN1CCC(C(=O)Nc2cnc3[nH]cc(-c4ccc5ncccc5c4)c3c2)CC1] | 648 |
| 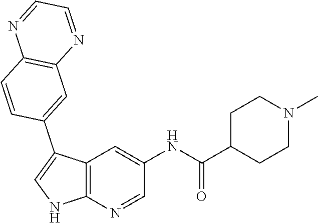 [SMILES: CN1CCC(C(=O)Nc2cnc3[nH]cc(-c4ccc5nccnc5c4)c3c2)CC1] | 649 |
| 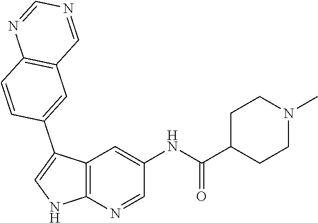 [SMILES: CN1CCC(C(=O)Nc2cnc3[nH]cc(-c4ccc5ncncc5c4)c3c2)CC1] | 650 |
| 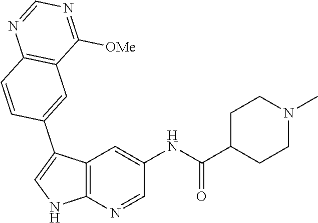 [SMILES: COc1ncnc2ccc(-c3c[nH]c4ncc(NC(=O)C5CCN(C)CC5)cc34)cc12] | 651 |
| 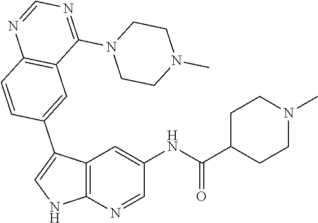 [SMILES: CN1CCC(C(=O)Nc2cnc3[nH]cc(-c4ccc5ncnc(N6CCN(C)CC6)c5c4)c3c2)CC1] | 652 |
| 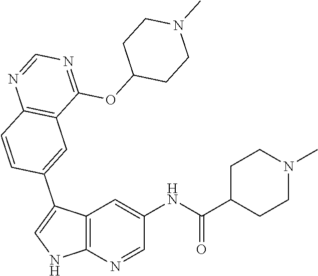 [SMILES: CN1CCC(Oc2ncnc3ccc(-c4c[nH]c5ncc(NC(=O)C6CCN(C)CC6)cc45)cc23)CC1] | 653 |
| 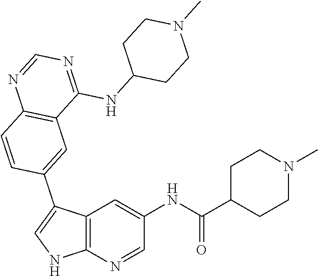 [SMILES: CN1CCC(Nc2ncnc3ccc(-c4c[nH]c5ncc(NC(=O)C6CCN(C)CC6)cc45)cc23)CC1] | 654 |
| 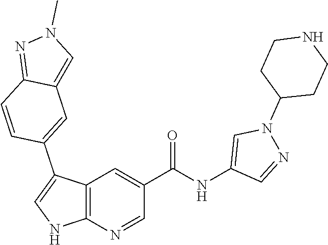 [SMILES: Cn1cc2cc(-c3c[nH]c4ncc(C(=O)Nc5cnn(C6CCNCC6)c5)cc34)ccc2n1] | 655 |
| 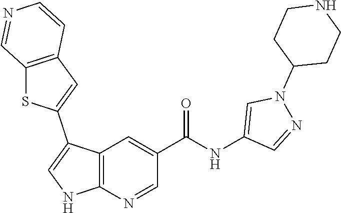 [SMILES: O=C(Nc1cnn(C2CCNCC2)c1)c1cnc2[nH]cc(-c3cc4ccncc4s3)c2c1] | 656 |
| 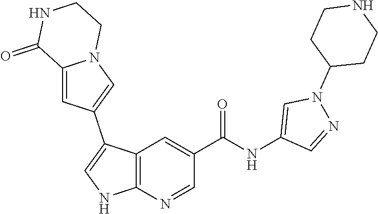 [SMILES: O=C(Nc1cnn(C2CCNCC2)c1)c1cnc2[nH]cc(-c3cc4n(c3)CCNC4=O)c2c1] | 657 |
| 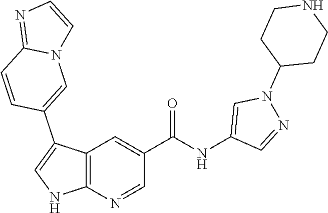 [SMILES: O=C(Nc1cnn(C2CCNCC2)c1)c1cnc2[nH]cc(-c3ccc4nccn4c3)c2c1] | 658 |
| 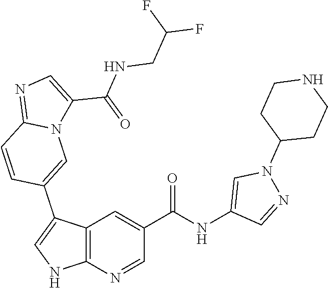 [SMILES: O=C(Nc1cnn(C2CCNCC2)c1)c1cnc2[nH]cc(-c3ccc4ncc(C(=O)NCC(F)F)n4c3)c2c1] | 659 |
| 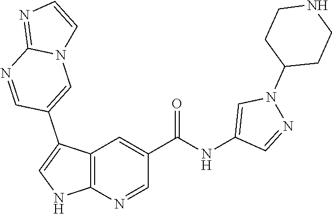 [SMILES: O=C(Nc1cnn(C2CCNCC2)c1)c1cnc2[nH]cc(-c3cnc4nccn4c3)c2c1] | 660 |
| 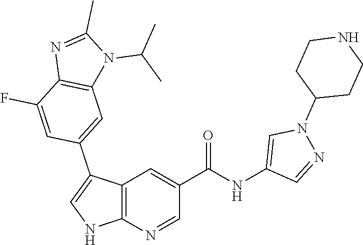 [SMILES: Cc1nc2c(F)cc(-c3c[nH]c4ncc(C(=O)Nc5cnn(C6CCNCC6)c5)cc34)cc2n1C(C)C] | 661 |
| 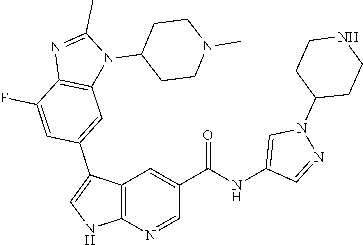 [SMILES: Cc1nc2c(F)cc(-c3c[nH]c4ncc(C(=O)Nc5cnn(C6CCNCC6)c5)cc34)cc2n1C1CCN(C)CC1] | 662 |
| 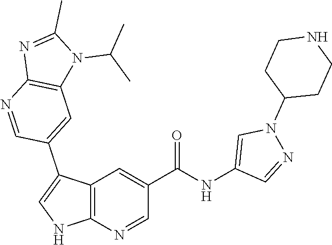 [SMILES: Cc1nc2ncc(-c3c[nH]c4ncc(C(=O)Nc5cnn(C6CCNCC6)c5)cc34)cc2n1C(C)C] | 663 |
| 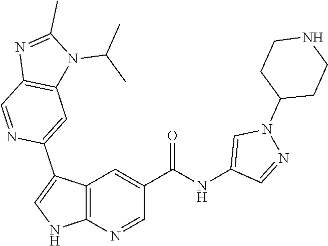 [SMILES: Cc1nc2cnc(-c3c[nH]c4ncc(C(=O)Nc5cnn(C6CCNCC6)c5)cc34)cc2n1C(C)C] | 664 |
| 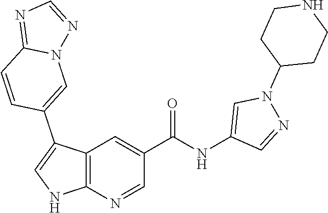 [SMILES: O=C(Nc1cnn(C2CCNCC2)c1)c1cnc2[nH]cc(-c3ccc4ncnn4c3)c2c1] | 665 |
| 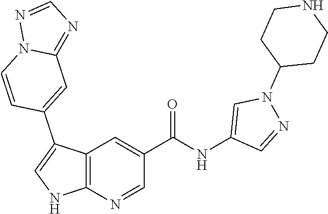 [SMILES: O=C(Nc1cnn(C2CCNCC2)c1)c1cnc2[nH]cc(-c3ccn4ncnc4c3)c2c1] | 666 |
| 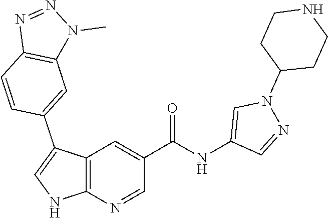 [SMILES: Cn1nnc2ccc(-c3c[nH]c4ncc(C(=O)Nc5cnn(C6CCNCC6)c5)cc34)cc21] | 667 |
| 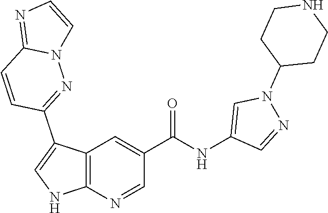 [SMILES: O=C(Nc1cnn(C2CCNCC2)c1)c1cnc2[nH]cc(-c3ccc4nccn4n3)c2c1] | 668 |
| 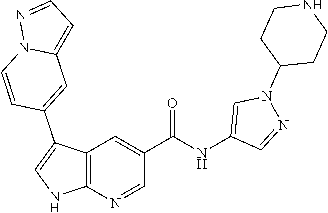 [SMILES: O=C(Nc1cnn(C2CCNCC2)c1)c1cnc2[nH]cc(-c3ccn4nccc4c3)c2c1] | 669 |
| 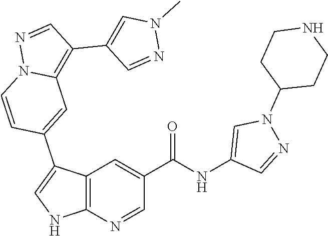 [SMILES: Cn1cc(-c2cnn3ccc(-c4c[nH]c5ncc(C(=O)Nc6cnn(C7CCNCC7)c6)cc45)cc23)cn1] | 670 |
| 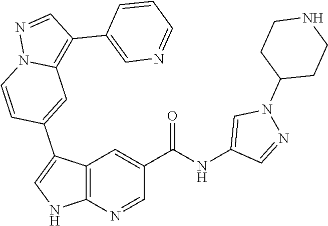 [SMILES: O=C(Nc1cnn(C2CCNCC2)c1)c1cnc2[nH]cc(-c3ccn4ncc(-c5cccnc5)c4c3)c2c1] | 671 |
| 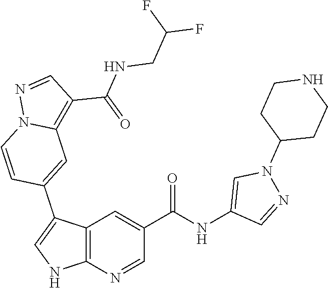 [SMILES: O=C(Nc1cnn(C2CCNCC2)c1)c1cnc2[nH]cc(-c3ccn4ncc(C(=O)NCC(F)F)c4c3)c2c1] | 672 |
| 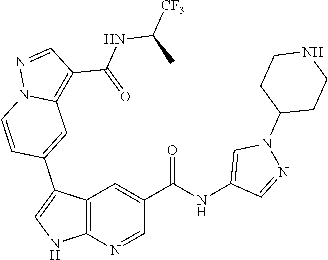 [SMILES: C[C@@H](NC(=O)c1cnn2ccc(-c3c[nH]c4ncc(C(=O)Nc5cnn(C6CCNCC6)c5)cc34)cc12)C(F)(F)F] | 673 |
| 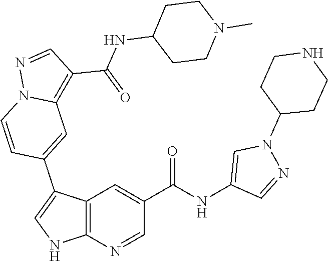 [SMILES: CN1CCC(NC(=O)c2cnn3ccc(-c4c[nH]c5ncc(C(=O)Nc6cnn(C7CCNCC7)c6)cc45)cc23)CC1] | 674 |
| 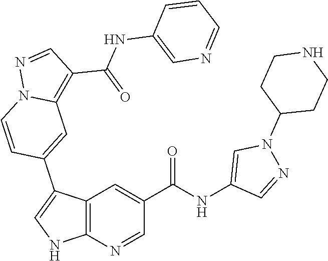 [SMILES: O=C(Nc1cnn(C2CCNCC2)c1)c1cnc2[nH]cc(-c3ccn4ncc(C(=O)Nc5cccnc5)c4c3)c2c1] | 675 |
| 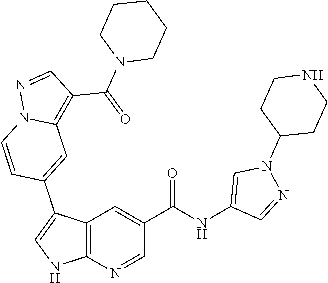 [SMILES: O=C(Nc1cnn(C2CCNCC2)c1)c1cnc2[nH]cc(-c3ccn4ncc(C(=O)N5CCCCC5)c4c3)c2c1] | 676 |
| 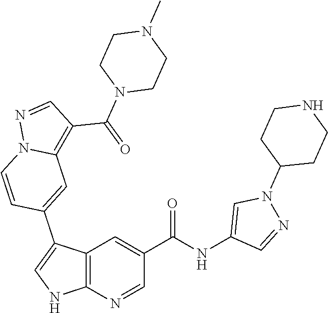 [SMILES: CN1CCN(C(=O)c2cnn3ccc(-c4c[nH]c5ncc(C(=O)Nc6cnn(C7CCNCC7)c6)cc45)cc23)CC1] | 677 |
| 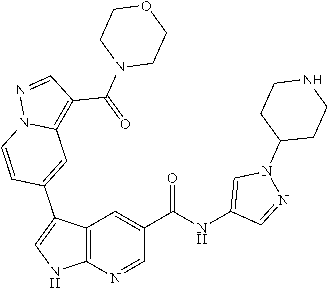 [SMILES: O=C(Nc1cnn(C2CCNCC2)c1)c1cnc2[nH]cc(-c3ccn4ncc(C(=O)N5CCOCC5)c4c3)c2c1] | 678 |
| 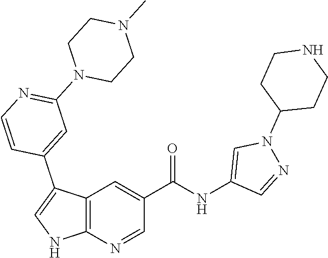 [SMILES: CN1CCN(c2cc(-c3c[nH]c4ncc(C(=O)Nc5cnn(C6CCNCC6)c5)cc34)ccn2)CC1] | 679 |
| 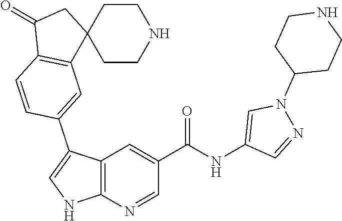 [SMILES: O=C(Nc1cnn(C2CCNCC2)c1)c1cnc2[nH]cc(-c3ccc4c(c3)C3(CCNCC3)CC4=O)c2c1] | 680 |
| 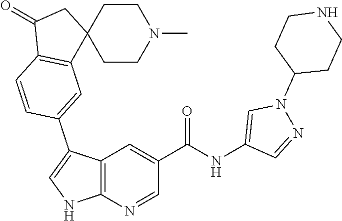 [SMILES: CN1CCC2(CC1)CC(=O)c1ccc(-c3c[nH]c4ncc(C(=O)Nc5cnn(C6CCNCC6)c5)cc34)cc12] | 681 |
| 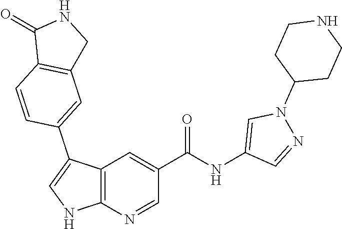 [SMILES: O=C(Nc1cnn(C2CCNCC2)c1)c1cnc2[nH]cc(-c3ccc4c(c3)CNC4=O)c2c1] | 682 |
| 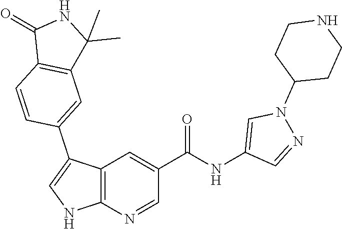 [SMILES: CC1(C)NC(=O)c2ccc(-c3c[nH]c4ncc(C(=O)Nc5cnn(C6CCNCC6)c5)cc34)cc21] | 683 |
| 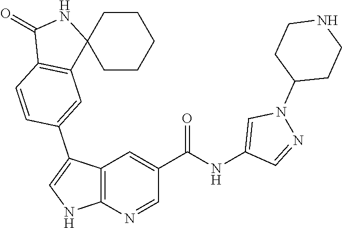 [SMILES: O=C(Nc1cnn(C2CCNCC2)c1)c1cnc2[nH]cc(-c3ccc4c(c3)C3(CCCCC3)NC4=O)c2c1] | 684 |
| 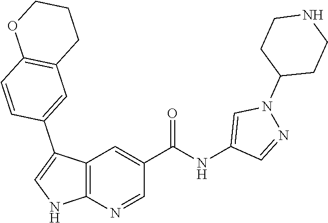 [SMILES: O=C(Nc1cnn(C2CCNCC2)c1)c1cnc2[nH]cc(-c3ccc4c(c3)CCCO4)c2c1] | 685 |
| 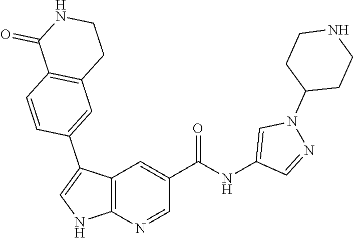 [SMILES: O=C(Nc1cnn(C2CCNCC2)c1)c1cnc2[nH]cc(-c3ccc4c(c3)CCNC4=O)c2c1] | 686 |
| 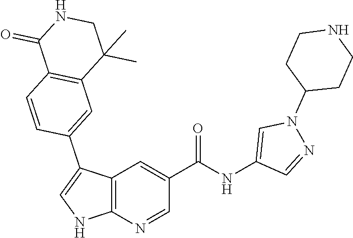 [SMILES: CC1(C)CNC(=O)c2ccc(-c3c[nH]c4ncc(C(=O)Nc5cnn(C6CCNCC6)c5)cc34)cc21] | 687 |
| 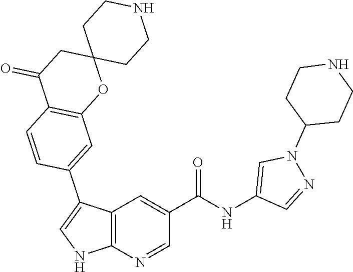 [SMILES: O=C(Nc1cnn(C2CCNCC2)c1)c1cnc2[nH]cc(-c3ccc4c(c3)OC3(CCNCC3)CC4=O)c2c1] | 688 |
| 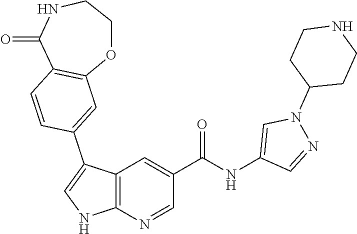 [SMILES: O=C(Nc1cnn(C2CCNCC2)c1)c1cnc2[nH]cc(-c3ccc4c(c3)OCCNC4=O)c2c1] | 689 |
| 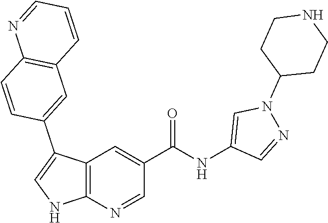 [SMILES: O=C(Nc1cnn(C2CCNCC2)c1)c1cnc2[nH]cc(-c3ccc4ncccc4c3)c2c1] | 690 |
| 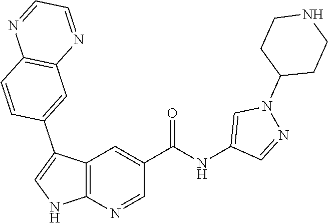 [SMILES: O=C(Nc1cnn(C2CCNCC2)c1)c1cnc2[nH]cc(-c3ccc4nccnc4c3)c2c1] | 691 |
| 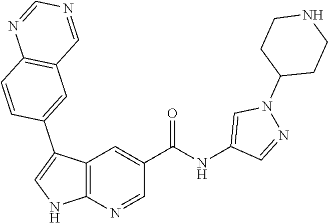 [SMILES: O=C(Nc1cnn(C2CCNCC2)c1)c1cnc2[nH]cc(-c3ccc4ncncc4c3)c2c1] | 692 |
| 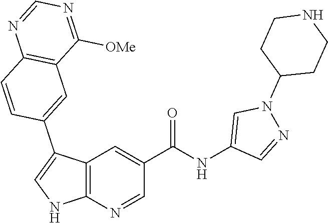 [SMILES: COc1ncnc2ccc(-c3c[nH]c4ncc(C(=O)Nc5cnn(C6CCNCC6)c5)cc34)cc12] | 693 |
| 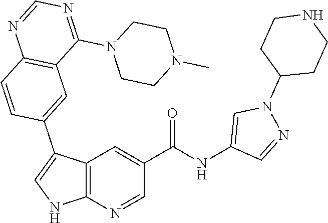 [SMILES: CN1CCN(c2ncnc3ccc(-c4c[nH]c5ncc(C(=O)Nc6cnn(C7CCNCC7)c6)cc45)cc23)CC1] | 694 |
| 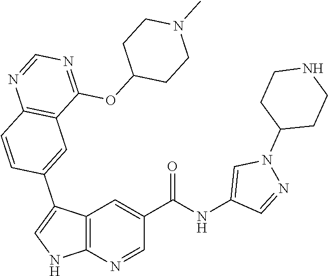 [SMILES: CN1CCC(Oc2ncnc3ccc(-c4c[nH]c5ncc(C(=O)Nc6cnn(C7CCNCC7)c6)cc45)cc23)CC1] | 695 |
| 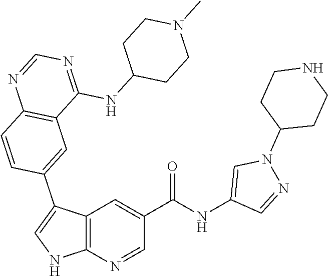 [SMILES: CN1CCC(Nc2ncnc3ccc(-c4c[nH]c5ncc(C(=O)Nc6cnn(C7CCNCC7)c6)cc45)cc23)CC1] | 696 |
| 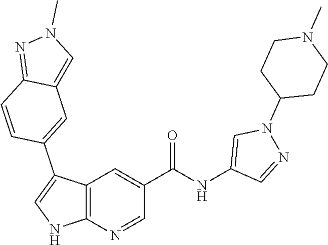 [SMILES: CN1CCC(n2cc(NC(=O)c3cnc4[nH]cc(-c5ccc6nn(C)cc6c5)c4c3)cn2)CC1] | 697 |
| 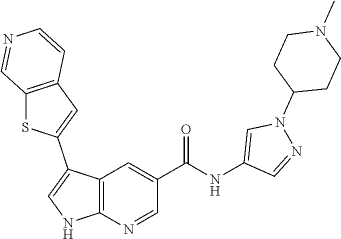 [SMILES: CN1CCC(n2cc(NC(=O)c3cnc4[nH]cc(-c5cc6ccncc6s5)c4c3)cn2)CC1] | 698 |
| 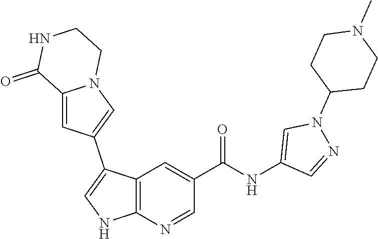 [SMILES: CN1CCC(n2cc(NC(=O)c3cnc4[nH]cc(-c5cc6n(c5)CCNC6=O)c4c3)cn2)CC1] | 699 |
| 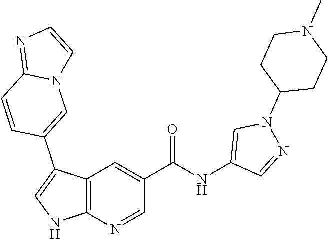 [SMILES: CN1CCC(n2cc(NC(=O)c3cnc4[nH]cc(-c5ccc6nccn6c5)c4c3)cn2)CC1] | 700 |
| 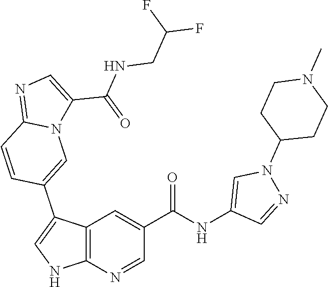 [SMILES: CN1CCC(n2cc(NC(=O)c3cnc4[nH]cc(-c5ccc6ncc(C(=O)NCC(F)F)n6c5)c4c3)cn2)CC1] | 701 |
| 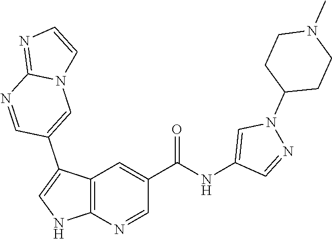 [SMILES: CN1CCC(n2cc(NC(=O)c3cnc4[nH]cc(-c5cnc6nccn6c5)c4c3)cn2)CC1] | 702 |
| 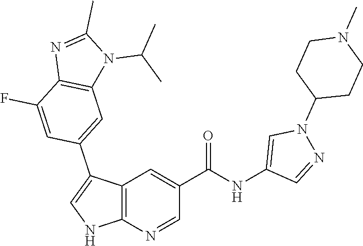 [SMILES: Cc1nc2c(F)cc(-c3c[nH]c4ncc(C(=O)Nc5cnn(C6CCN(C)CC6)c5)cc34)cc2n1C(C)C] | 703 |
| 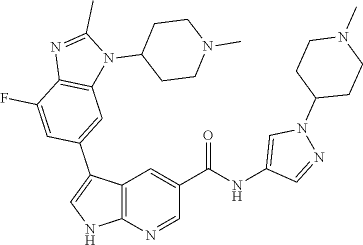 [SMILES: Cc1nc2c(F)cc(-c3c[nH]c4ncc(C(=O)Nc5cnn(C6CCN(C)CC6)c5)cc34)cc2n1C1CCN(C)CC1] | 704 |
| 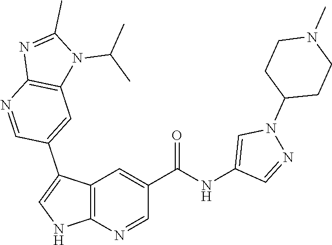 [SMILES: Cc1nc2ncc(-c3c[nH]c4ncc(C(=O)Nc5cnn(C6CCN(C)CC6)c5)cc34)cc2n1C(C)C] | 705 |
| 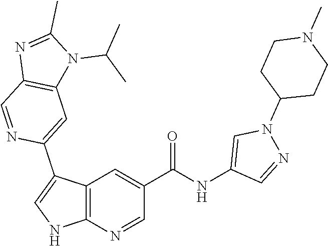 [SMILES: Cc1nc2cnc(-c3c[nH]c4ncc(C(=O)Nc5cnn(C6CCN(C)CC6)c5)cc34)cc2n1C(C)C] | 706 |
| 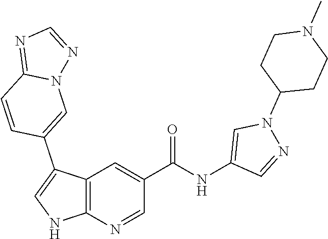 [SMILES: CN1CCC(n2cc(NC(=O)c3cnc4[nH]cc(-c5ccc6ncnn6c5)c4c3)cn2)CC1] | 707 |
| 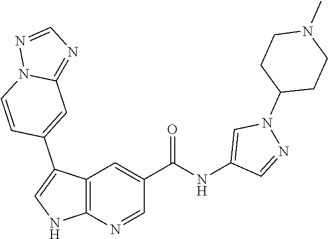 [SMILES: CN1CCC(n2cc(NC(=O)c3cnc4[nH]cc(-c5ccn6ncnc6c5)c4c3)cn2)CC1] | 708 |
| 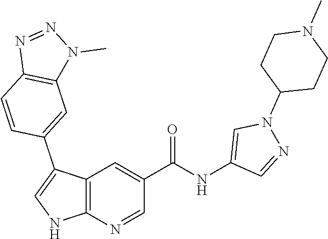 [SMILES: CN1CCC(n2cc(NC(=O)c3cnc4[nH]cc(-c5ccc6nnn(C)c6c5)c4c3)cn2)CC1] | 709 |
| 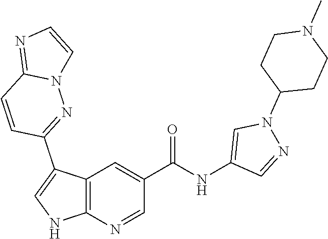 [SMILES: CN1CCC(n2cc(NC(=O)c3cnc4[nH]cc(-c5ccc6nccn6n5)c4c3)cn2)CC1] | 710 |
| 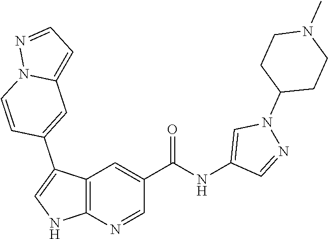 [SMILES: CN1CCC(n2cc(NC(=O)c3cnc4[nH]cc(-c5ccn6nccc6c5)c4c3)cn2)CC1] | 711 |
| 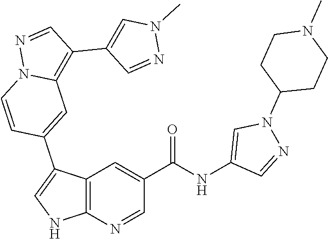 [SMILES: CN1CCC(n2cc(NC(=O)c3cnc4[nH]cc(-c5ccn6ncc(-c7cnn(C)c7)c6c5)c4c3)cn2)CC1] | 712 |
| 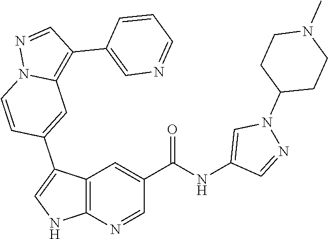 [SMILES: CN1CCC(n2cc(NC(=O)c3cnc4[nH]cc(-c5ccn6ncc(-c7cccnc7)c6c5)c4c3)cn2)CC1] | 713 |
| 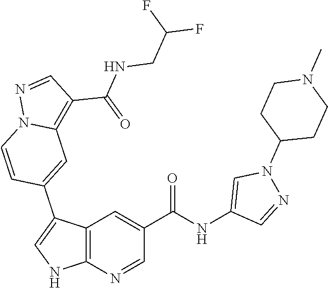 [SMILES: CN1CCC(n2cc(NC(=O)c3cnc4[nH]cc(-c5ccn6ncc(C(=O)NCC(F)F)c6c5)c4c3)cn2)CC1] | 714 |
| 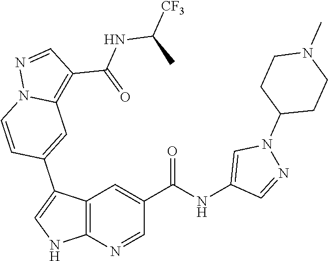 [SMILES: C[C@@H](NC(=O)c1cnn2ccc(-c3c[nH]c4ncc(C(=O)Nc5cnn(C6CCN(C)CC6)c5)cc34)cc12)C(F)(F)F] | 715 |
| 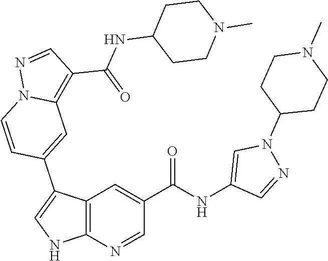 [SMILES: CN1CCC(NC(=O)c2cnn3ccc(-c4c[nH]c5ncc(C(=O)Nc6cnn(C7CCN(C)CC7)c6)cc45)cc23)CC1] | 716 |
| 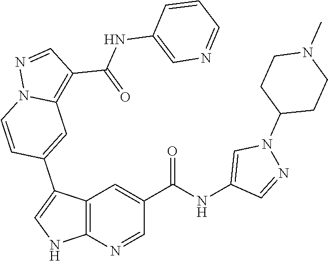 [SMILES: CN1CCC(n2cc(NC(=O)c3cnc4[nH]cc(-c5ccn6ncc(C(=O)Nc7cccnc7)c6c5)c4c3)cn2)CC1] | 717 |
| 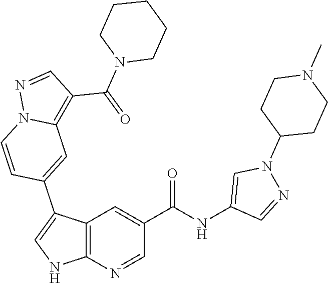 [SMILES: CN1CCC(n2cc(NC(=O)c3cnc4[nH]cc(-c5ccn6ncc(C(=O)N7CCCCC7)c6c5)c4c3)cn2)CC1] | 718 |
| 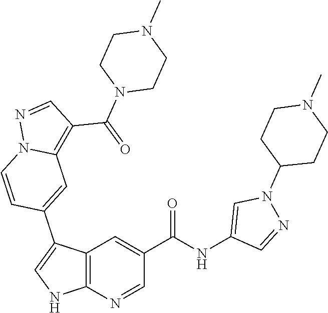 [SMILES: CN1CCC(n2cc(NC(=O)c3cnc4[nH]cc(-c5ccn6ncc(C(=O)N7CCN(C)CC7)c6c5)c4c3)cn2)CC1] | 719 |
| 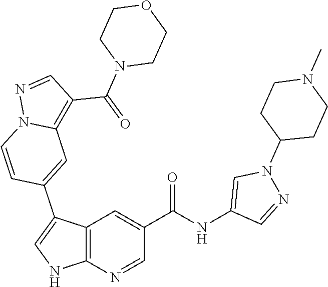 [SMILES: CN1CCC(n2cc(NC(=O)c3cnc4[nH]cc(-c5ccn6ncc(C(=O)N7CCOCC7)c6c5)c4c3)cn2)CC1] | 720 |
| 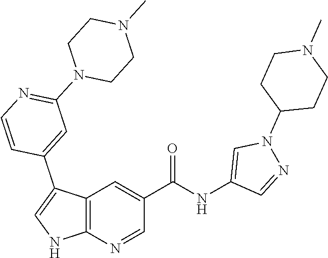 [SMILES: CN1CCC(n2cc(NC(=O)c3cnc4[nH]cc(-c5ccnc(N6CCN(C)CC6)c5)c4c3)cn2)CC1] | 721 |
| 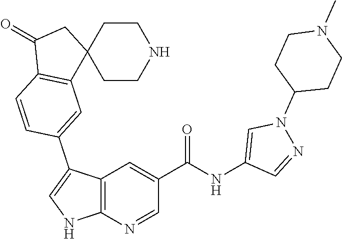 [SMILES: CN1CCC(n2cc(NC(=O)c3cnc4[nH]cc(-c5ccc6c(c5)C5(CCNCC5)CC6=O)c4c3)cn2)CC1] | 722 |
| 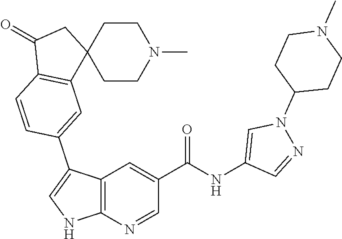 [SMILES: CN1CCC(n2cc(NC(=O)c3cnc4[nH]cc(-c5ccc6c(c5)C5(CCN(C)CC5)CC6=O)c4c3)cn2)CC1] | 723 |
| 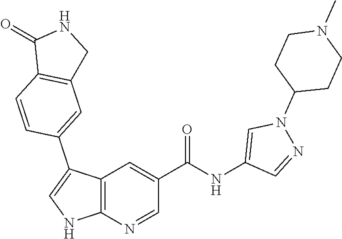 [SMILES: CN1CCC(n2cc(NC(=O)c3cnc4[nH]cc(-c5ccc6c(c5)CNC6=O)c4c3)cn2)CC1] | 724 |
| 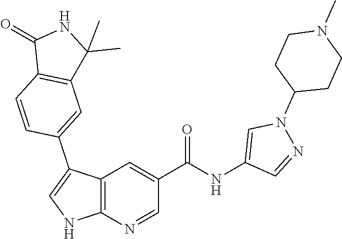 [SMILES: CN1CCC(n2cc(NC(=O)c3cnc4[nH]cc(-c5ccc6c(c5)C(C)(C)NC6=O)c4c3)cn2)CC1] | 725 |
| 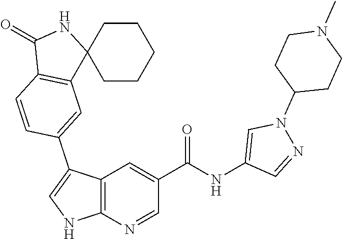 [SMILES: CN1CCC(n2cc(NC(=O)c3cnc4[nH]cc(-c5ccc6c(c5)C5(CCCCC5)NC6=O)c4c3)cn2)CC1] | 726 |
| 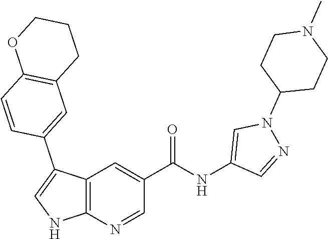 [SMILES: CN1CCC(n2cc(NC(=O)c3cnc4[nH]cc(-c5ccc6c(c5)CCCO6)c4c3)cn2)CC1] | 727 |
| 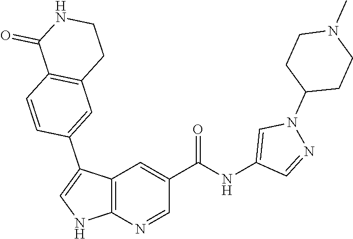 [SMILES: CN1CCC(n2cc(NC(=O)c3cnc4[nH]cc(-c5ccc6c(c5)CCNC6=O)c4c3)cn2)CC1] | 728 |
| 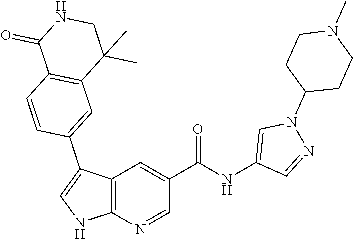 [SMILES: CN1CCC(n2cc(NC(=O)c3cnc4[nH]cc(-c5ccc6c(c5)C(C)(C)CNC6=O)c4c3)cn2)CC1] | 729 |
| 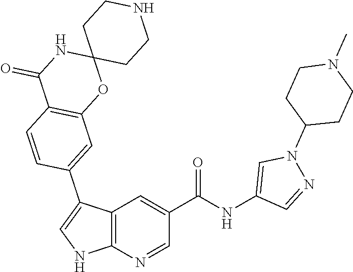 [SMILES: CN1CCC(n2cc(NC(=O)c3cnc4[nH]cc(-c5ccc6c(c5)OC5(CCNCC5)NC6=O)c4c3)cn2)CC1] | 730 |
| 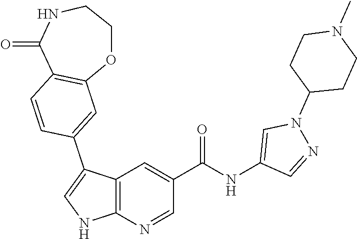 [SMILES: CN1CCC(n2cc(NC(=O)c3cnc4[nH]cc(-c5ccc6c(c5)OCCNC6=O)c4c3)cn2)CC1] | 731 |
| 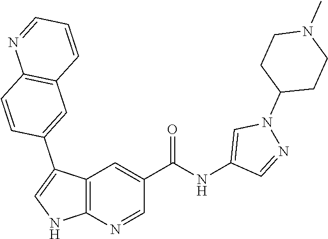 [SMILES: CN1CCC(n2cc(NC(=O)c3cnc4[nH]cc(-c5ccc6ncccc6c5)c4c3)cn2)CC1] | 732 |
| 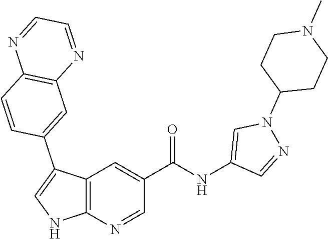 [SMILES: CN1CCC(n2cc(NC(=O)c3cnc4[nH]cc(-c5ccc6nccnc6c5)c4c3)cn2)CC1] | 733 |
| 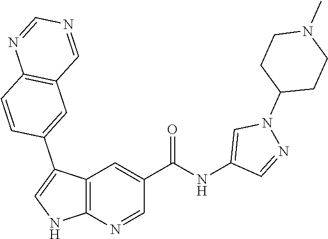 [SMILES: CN1CCC(n2cc(NC(=O)c3cnc4[nH]cc(-c5ccc6ncncc6c5)c4c3)cn2)CC1] | 734 |
| 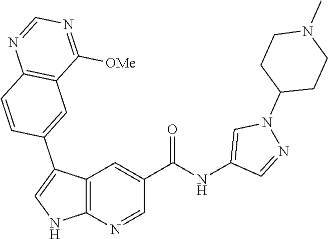 [SMILES: COc1ncnc2ccc(-c3c[nH]c4ncc(C(=O)Nc5cnn(C6CCN(C)CC6)c5)cc34)cc12] | 735 |
| 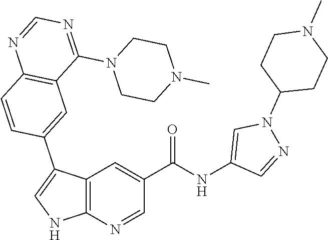 [SMILES: CN1CCC(n2cc(NC(=O)c3cnc4[nH]cc(-c5ccc6ncnc(N7CCN(C)CC7)c6c5)c4c3)cn2)CC1] | 736 |
| 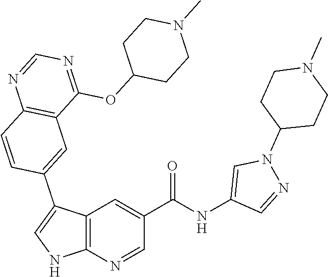 [SMILES: CN1CCC(Oc2ncnc3ccc(-c4c[nH]c5ncc(C(=O)Nc6cnn(C7CCN(C)CC7)c6)cc45)cc23)CC1] | 737 |
| 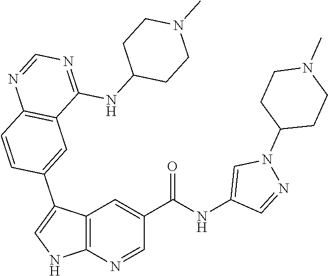 [SMILES: CN1CCC(Nc2ncnc3ccc(-c4c[nH]c5ncc(C(=O)Nc6cnn(C7CCN(C)CC7)c6)cc45)cc23)CC1] | 738 |
| 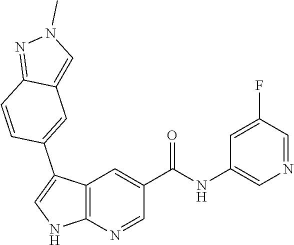 [SMILES: Cn1cc2cc(-c3c[nH]c4ncc(C(=O)Nc5cncc(F)c5)cc34)ccc2n1] | 739 |
| 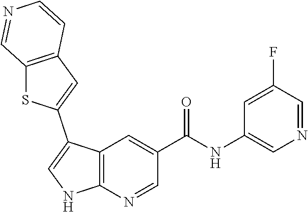 [SMILES: O=C(Nc1cncc(F)c1)c1cnc2[nH]cc(-c3cc4ccncc4s3)c2c1] | 740 |
| 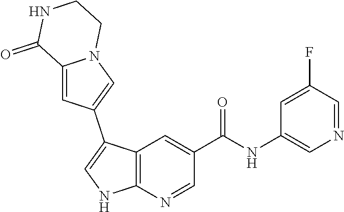 [SMILES: O=C(Nc1cncc(F)c1)c1cnc2[nH]cc(-c3cc4n(c3)CCNC4=O)c2c1] | 741 |
| 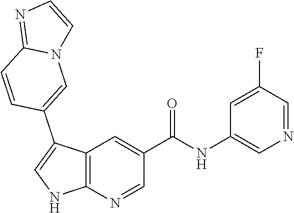 [SMILES: O=C(Nc1cncc(F)c1)c1cnc2[nH]cc(-c3ccc4nccn4c3)c2c1] | 742 |
| 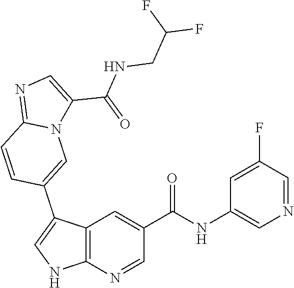 [SMILES: O=C(Nc1cncc(F)c1)c1cnc2[nH]cc(-c3ccc4ncc(C(=O)NCC(F)F)n4c3)c2c1] | 743 |
| 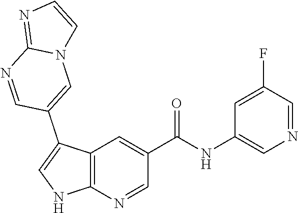 [SMILES: O=C(Nc1cncc(F)c1)c1cnc2[nH]cc(-c3cnc4nccn4c3)c2c1] | 744 |
| 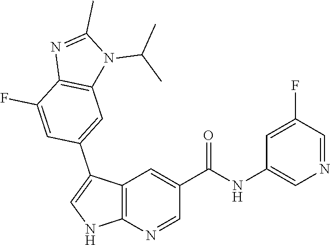 [SMILES: Cc1nc2c(F)cc(-c3c[nH]c4ncc(C(=O)Nc5cncc(F)c5)cc34)cc2n1C(C)C] | 745 |
| 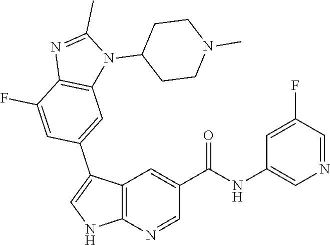 [SMILES: Cc1nc2c(F)cc(-c3c[nH]c4ncc(C(=O)Nc5cncc(F)c5)cc34)cc2n1C1CCN(C)CC1] | 746 |
| 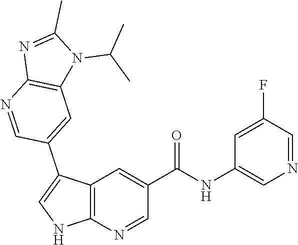 [SMILES: Cc1nc2ncc(-c3c[nH]c4ncc(C(=O)Nc5cncc(F)c5)cc34)cc2n1C(C)C] | 747 |
| 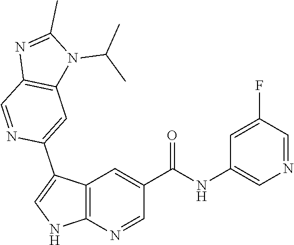 [SMILES: Cc1nc2cnc(-c3c[nH]c4ncc(C(=O)Nc5cncc(F)c5)cc34)cc2n1C(C)C] | 748 |
| 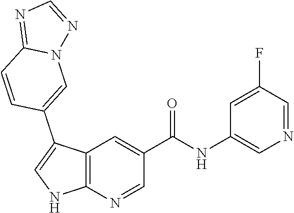 [SMILES: O=C(Nc1cncc(F)c1)c1cnc2[nH]cc(-c3ccc4ncnn4c3)c2c1] | 749 |
| 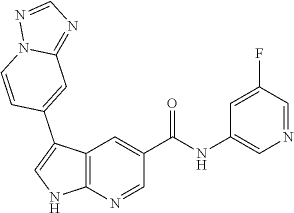 [SMILES: O=C(Nc1cncc(F)c1)c1cnc2[nH]cc(-c3ccn4ncnc4c3)c2c1] | 750 |
| 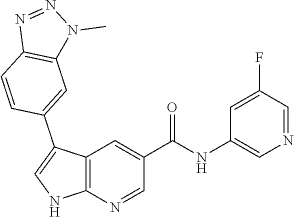 [SMILES: Cn1nnc2ccc(-c3c[nH]c4ncc(C(=O)Nc5cncc(F)c5)cc34)cc21] | 751 |
| 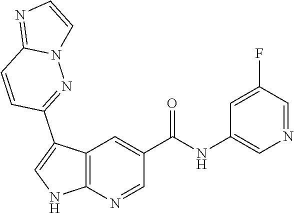 [SMILES: O=C(Nc1cncc(F)c1)c1cnc2[nH]cc(-c3ccc4nccn4n3)c2c1] | 752 |
| 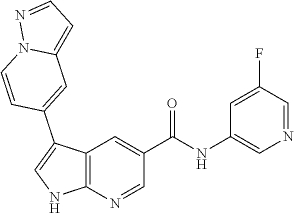 [SMILES: O=C(Nc1cncc(F)c1)c1cnc2[nH]cc(-c3ccn4nccc4c3)c2c1] | 753 |
| 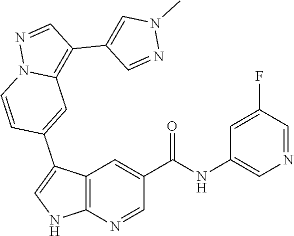 [SMILES: Cn1cc(-c2cnn3ccc(-c4c[nH]c5ncc(C(=O)Nc6cncc(F)c6)cc45)cc23)cn1] | 754 |
| 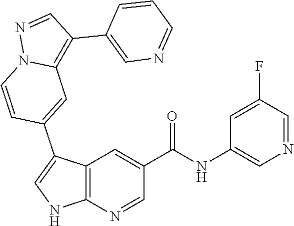 [SMILES: O=C(Nc1cncc(F)c1)c1cnc2[nH]cc(-c3ccn4ncc(-c5cccnc5)c4c3)c2c1] | 755 |
| 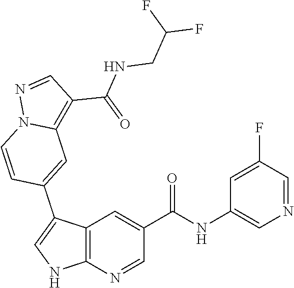 [SMILES: O=C(Nc1cncc(F)c1)c1cnc2[nH]cc(-c3ccn4ncc(C(=O)NCC(F)F)c4c3)c2c1] | 756 |
| 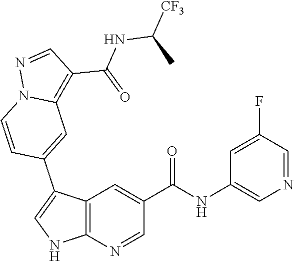 [SMILES: C[C@@H](NC(=O)c1cnn2ccc(-c3c[nH]c4ncc(C(=O)Nc5cncc(F)c5)cc34)cc12)C(F)(F)F] | 757 |
| 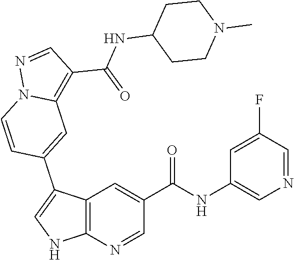 [SMILES: CN1CCC(NC(=O)c2cnn3ccc(-c4c[nH]c5ncc(C(=O)Nc6cncc(F)c6)cc45)cc23)CC1] | 758 |
| 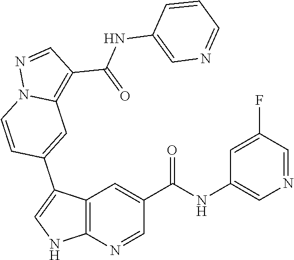 [SMILES: O=C(Nc1cncc(F)c1)c1cnc2[nH]cc(-c3ccn4ncc(C(=O)Nc5cccnc5)c4c3)c2c1] | 759 |
| 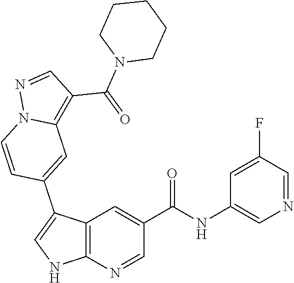 [SMILES: O=C(Nc1cncc(F)c1)c1cnc2[nH]cc(-c3ccn4ncc(C(=O)N5CCCCC5)c4c3)c2c1] | 760 |
| 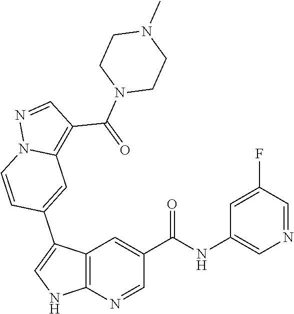 [SMILES: CN1CCN(C(=O)c2cnn3ccc(-c4c[nH]c5ncc(C(=O)Nc6cncc(F)c6)cc45)cc23)CC1] | 761 |
| 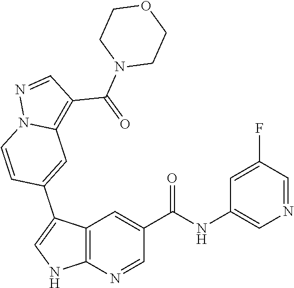 [SMILES: O=C(Nc1cncc(F)c1)c1cnc2[nH]cc(-c3ccn4ncc(C(=O)N5CCOCC5)c4c3)c2c1] | 762 |
| 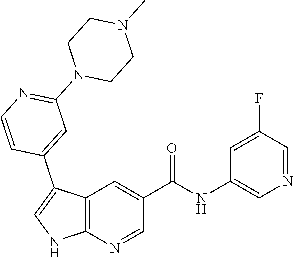 [SMILES: CN1CCN(c2cc(-c3c[nH]c4ncc(C(=O)Nc5cncc(F)c5)cc34)ccn2)CC1] | 763 |
| 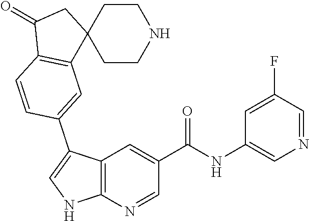 [SMILES: O=C(Nc1cncc(F)c1)c1cnc2[nH]cc(-c3ccc4c(c3)C3(CCNCC3)CC4=O)c2c1] | 764 |
| 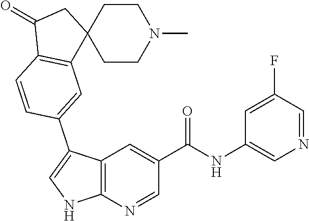 [SMILES: CN1CCC2(CC1)CC(=O)c1ccc(-c3c[nH]c4ncc(C(=O)Nc5cncc(F)c5)cc34)cc12] | 765 |
| 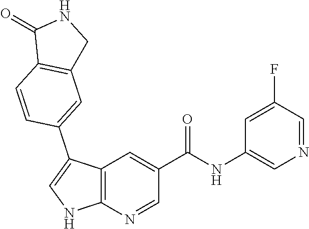 [SMILES: O=C(Nc1cncc(F)c1)c1cnc2[nH]cc(-c3ccc4c(c3)CNC4=O)c2c1] | 766 |
| 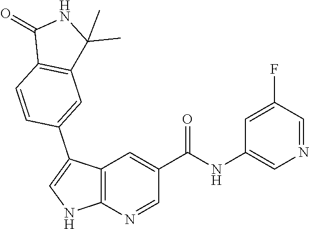 [SMILES: CC1(C)NC(=O)c2ccc(-c3c[nH]c4ncc(C(=O)Nc5cncc(F)c5)cc34)cc21] | 767 |
| 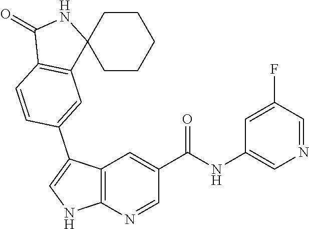 [SMILES: O=C(Nc1cncc(F)c1)c1cnc2[nH]cc(-c3ccc4c(c3)C3(CCCCC3)NC4=O)c2c1] | 768 |
| 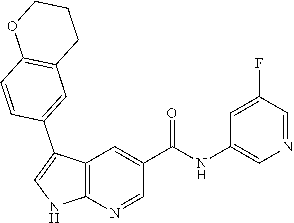 [SMILES: O=C(Nc1cncc(F)c1)c1cnc2[nH]cc(-c3ccc4c(c3)CCCO4)c2c1] | 769 |
| 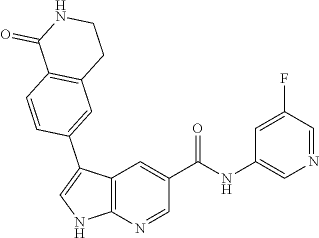 [SMILES: O=C(Nc1cncc(F)c1)c1cnc2[nH]cc(-c3ccc4c(c3)CCNC4=O)c2c1] | 770 |
| 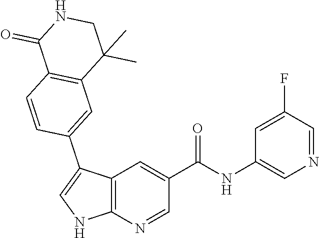 [SMILES: CC1(C)CNC(=O)c2ccc(-c3c[nH]c4ncc(C(=O)Nc5cncc(F)c5)cc34)cc21] | 771 |
| 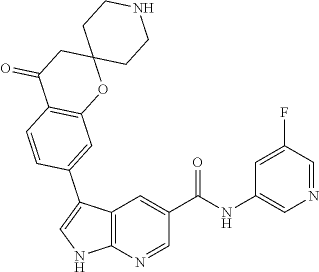 [SMILES: O=C(Nc1cncc(F)c1)c1cnc2[nH]cc(-c3ccc4c(c3)OC3(CCNCC3)CC4=O)c2c1] | 772 |
| 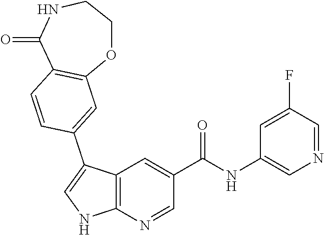 [SMILES: O=C(Nc1cncc(F)c1)c1cnc2[nH]cc(-c3ccc4c(c3)OCCNC4=O)c2c1] | 773 |
| 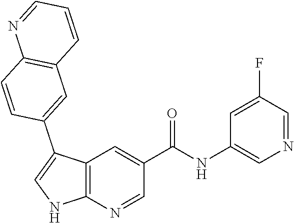 [SMILES: O=C(Nc1cncc(F)c1)c1cnc2[nH]cc(-c3ccc4ncccc4c3)c2c1] | 774 |
| 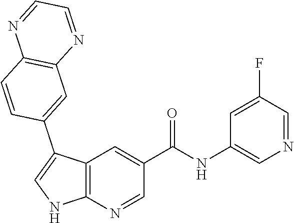 [SMILES: O=C(Nc1cncc(F)c1)c1cnc2[nH]cc(-c3ccc4nccnc4c3)c2c1] | 775 |
| 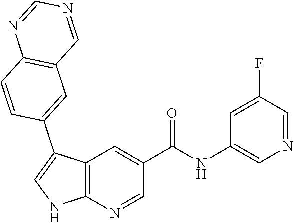 [SMILES: O=C(Nc1cncc(F)c1)c1cnc2[nH]cc(-c3ccc4ncncc4c3)c2c1] | 776 |
| 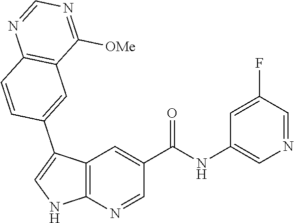 [SMILES: COc1ncnc2ccc(-c3c[nH]c4ncc(C(=O)Nc5cncc(F)c5)cc34)cc12] | 777 |
| 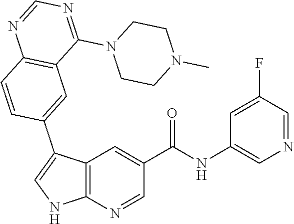 [SMILES: CN1CCN(c2ncnc3ccc(-c4c[nH]c5ncc(C(=O)Nc6cncc(F)c6)cc45)cc23)CC1] | 778 |
| 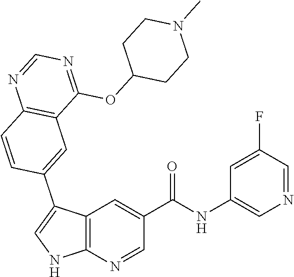 [SMILES: CN1CCC(Oc2ncnc3ccc(-c4c[nH]c5ncc(C(=O)Nc6cncc(F)c6)cc45)cc23)CC1] | 779 |
| 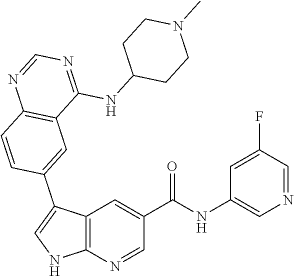 [SMILES: CN1CCC(Nc2ncnc3ccc(-c4c[nH]c5ncc(C(=O)Nc6cncc(F)c6)cc45)cc23)CC1] | 780 |
| 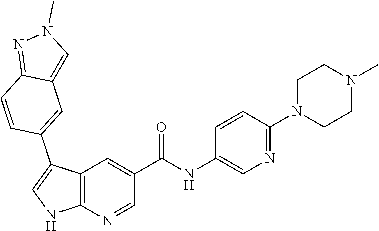 [SMILES: CN1CCN(c2ccc(NC(=O)c3cnc4[nH]cc(-c5ccc6nn(C)cc6c5)c4c3)cn2)CC1] | 781 |
| 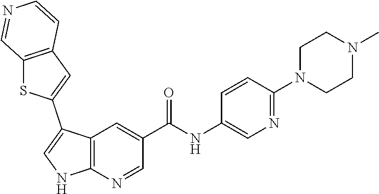 [SMILES: CN1CCN(c2ccc(NC(=O)c3cnc4[nH]cc(-c5cc6ccncc6s5)c4c3)cn2)CC1] | 782 |
| 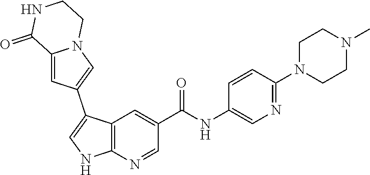 [SMILES: CN1CCN(c2ccc(NC(=O)c3cnc4[nH]cc(-c5cc6n(c5)CCNC6=O)c4c3)cn2)CC1] | 783 |
| 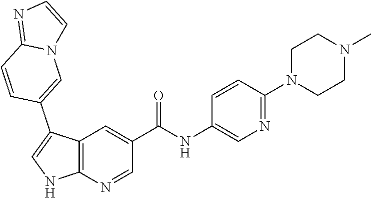 [SMILES: CN1CCN(c2ccc(NC(=O)c3cnc4[nH]cc(-c5ccc6nccn6c5)c4c3)cn2)CC1] | 784 |
| 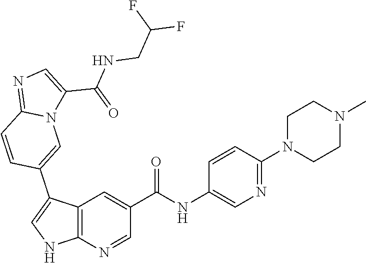 [SMILES: CN1CCN(c2ccc(NC(=O)c3cnc4[nH]cc(-c5ccc6ncc(C(=O)NCC(F)F)n6c5)c4c3)cn2)CC1] | 785 |
| 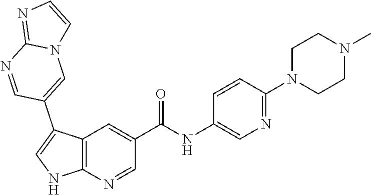 [SMILES: CN1CCN(c2ccc(NC(=O)c3cnc4[nH]cc(-c5cnc6nccn6c5)c4c3)cn2)CC1] | 786 |
| 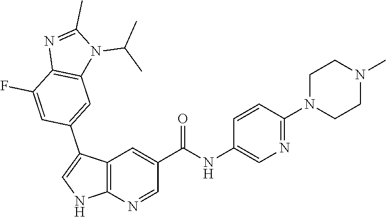 [SMILES: Cc1nc2c(F)cc(-c3c[nH]c4ncc(C(=O)Nc5ccc(N6CCN(C)CC6)nc5)cc34)cc2n1C(C)C] | 787 |
| 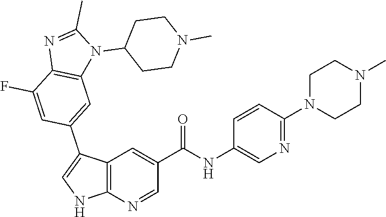 [SMILES: Cc1nc2c(F)cc(-c3c[nH]c4ncc(C(=O)Nc5ccc(N6CCN(C)CC6)nc5)cc34)cc2n1C1CCN(C)CC1] | 788 |
| 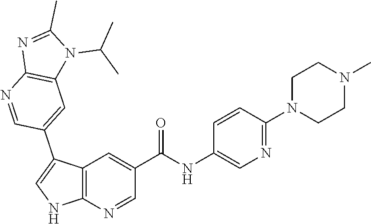 [SMILES: Cc1nc2ncc(-c3c[nH]c4ncc(C(=O)Nc5ccc(N6CCN(C)CC6)nc5)cc34)cc2n1C(C)C] | 789 |
| 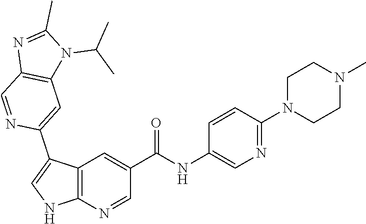 [SMILES: Cc1nc2cnc(-c3c[nH]c4ncc(C(=O)Nc5ccc(N6CCN(C)CC6)nc5)cc34)cc2n1C(C)C] | 790 |
| 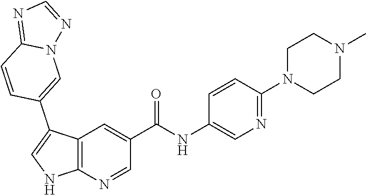 [SMILES: CN1CCN(c2ccc(NC(=O)c3cnc4[nH]cc(-c5ccc6ncnn6c5)c4c3)cn2)CC1] | 791 |
| 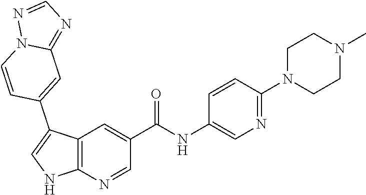 [SMILES: CN1CCN(c2ccc(NC(=O)c3cnc4[nH]cc(-c5ccn6ncnc6c5)c4c3)cn2)CC1] | 792 |
| 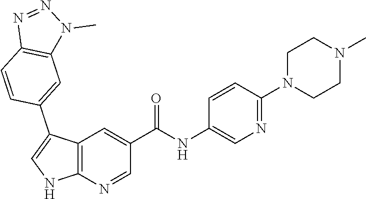 [SMILES: CN1CCN(c2ccc(NC(=O)c3cnc4[nH]cc(-c5ccc6nnn(C)c6c5)c4c3)cn2)CC1] | 793 |
| 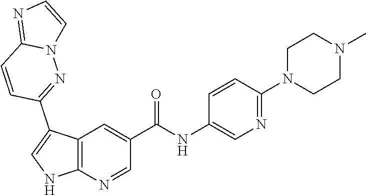 [SMILES: CN1CCN(c2ccc(NC(=O)c3cnc4[nH]cc(-c5ccc6nccn6n5)c4c3)cn2)CC1] | 794 |
| 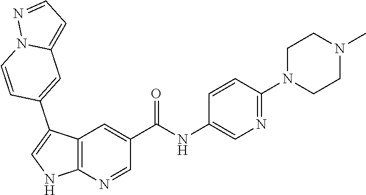 [SMILES: CN1CCN(c2ccc(NC(=O)c3cnc4[nH]cc(-c5ccn6nccc6c5)c4c3)cn2)CC1] | 795 |
| 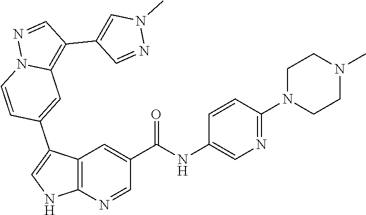 [SMILES: CN1CCN(c2ccc(NC(=O)c3cnc4[nH]cc(-c5ccn6ncc(-c7cnn(C)c7)c6c5)c4c3)cn2)CC1] | 796 |
| 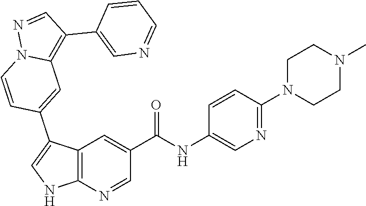 [SMILES: CN1CCN(c2ccc(NC(=O)c3cnc4[nH]cc(-c5ccn6ncc(-c7cccnc7)c6c5)c4c3)cn2)CC1] | 797 |
| 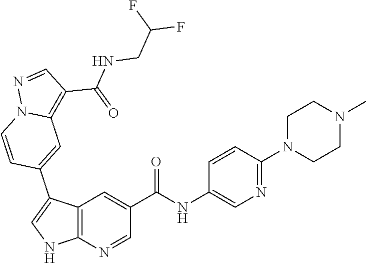 [SMILES: CN1CCN(c2ccc(NC(=O)c3cnc4[nH]cc(-c5ccn6ncc(C(=O)NCC(F)F)c6c5)c4c3)cn2)CC1] | 798 |
| 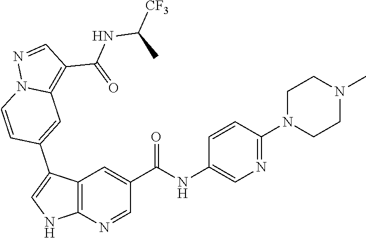 [SMILES: C[C@@H](NC(=O)c1cnn2ccc(-c3c[nH]c4ncc(C(=O)Nc5ccc(N6CCN(C)CC6)nc5)cc34)cc12)C(F)(F)F] | 799 |
| 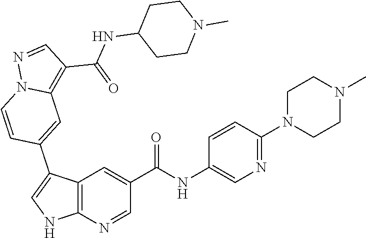 [SMILES: CN1CCC(NC(=O)c2cnn3ccc(-c4c[nH]c5ncc(C(=O)Nc6ccc(N7CCN(C)CC7)nc6)cc45)cc23)CC1] | 800 |
| 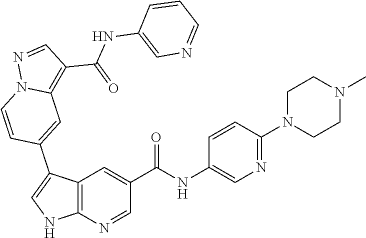 [SMILES: CN1CCN(c2ccc(NC(=O)c3cnc4[nH]cc(-c5ccn6ncc(C(=O)Nc7cccnc7)c6c5)c4c3)cn2)CC1] | 801 |
| 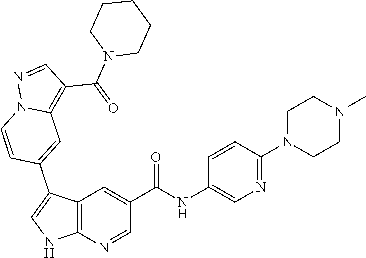 [SMILES: CN1CCN(c2ccc(NC(=O)c3cnc4[nH]cc(-c5ccn6ncc(C(=O)N7CCCCC7)c6c5)c4c3)cn2)CC1] | 802 |
| 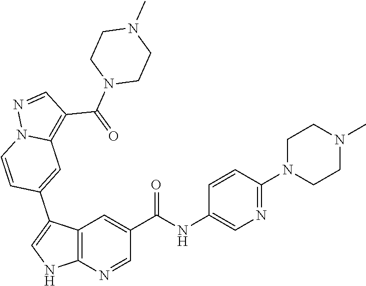 [SMILES: CN1CCN(C(=O)c2cnn3ccc(-c4c[nH]c5ncc(C(=O)Nc6ccc(N7CCN(C)CC7)nc6)cc45)cc23)CC1] | 803 |
| 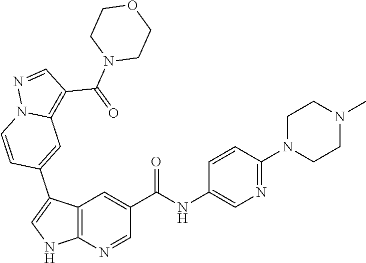 [SMILES: CN1CCN(c2ccc(NC(=O)c3cnc4[nH]cc(-c5ccn6ncc(C(=O)N7CCOCC7)c6c5)c4c3)cn2)CC1] | 804 |
| 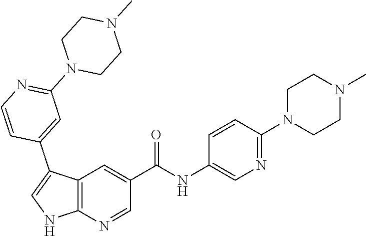 [SMILES: CN1CCN(c2ccc(NC(=O)c3cnc4[nH]cc(-c5ccnc(N6CCN(C)CC6)c5)c4c3)cn2)CC1] | 805 |
| 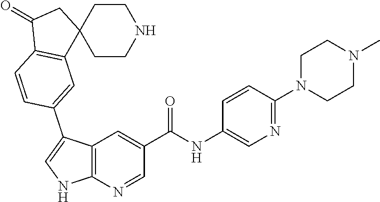 [SMILES: CN1CCN(c2ccc(NC(=O)c3cnc4[nH]cc(-c5ccc6c(c5)C5(CCNCC5)CC6=O)c4c3)cn2)CC1] | 806 |
| 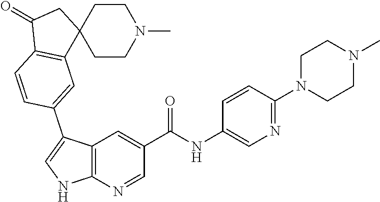 [SMILES: CN1CCN(c2ccc(NC(=O)c3cnc4[nH]cc(-c5ccc6c(c5)C5(CCN(C)CC5)CC6=O)c4c3)cn2)CC1] | 807 |
| 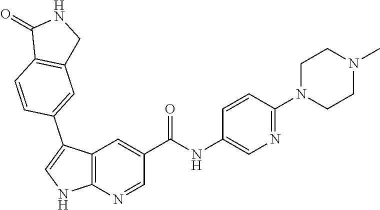 [SMILES: CN1CCN(c2ccc(NC(=O)c3cnc4[nH]cc(-c5ccc6c(c5)CNC6=O)c4c3)cn2)CC1] | 808 |
| 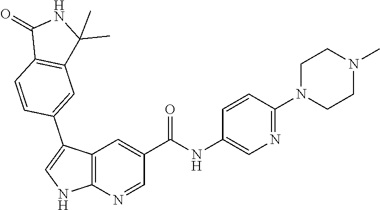 [SMILES: CN1CCN(c2ccc(NC(=O)c3cnc4[nH]cc(-c5ccc6c(c5)C(C)(C)NC6=O)c4c3)cn2)CC1] | 809 |
| 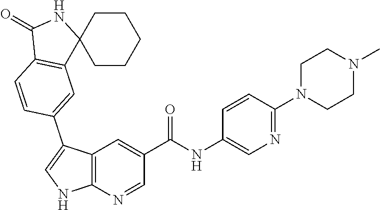 [SMILES: CN1CCN(c2ccc(NC(=O)c3cnc4[nH]cc(-c5ccc6c(c5)C5(CCCCC5)NC6=O)c4c3)cn2)CC1] | 810 |
| 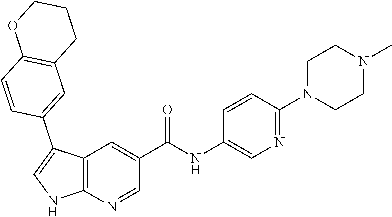 [SMILES: CN1CCN(c2ccc(NC(=O)c3cnc4[nH]cc(-c5ccc6c(c5)CCCO6)c4c3)cn2)CC1] | 811 |
| 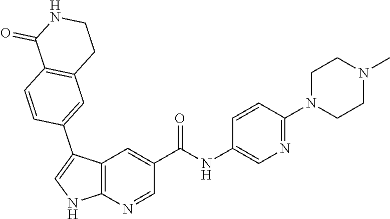 [SMILES: CN1CCN(c2ccc(NC(=O)c3cnc4[nH]cc(-c5ccc6c(c5)CCNC6=O)c4c3)cn2)CC1] | 812 |
| 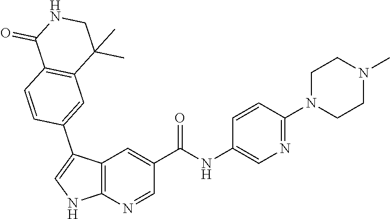 [SMILES: CN1CCN(c2ccc(NC(=O)c3cnc4[nH]cc(-c5ccc6c(c5)C(C)(C)CNC6=O)c4c3)cn2)CC1] | 813 |
| 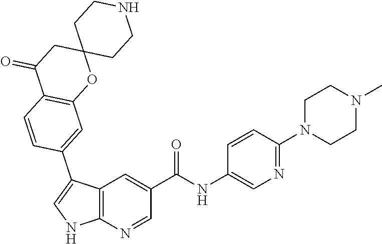 [SMILES: CN1CCN(c2ccc(NC(=O)c3cnc4[nH]cc(-c5ccc6c(c5)OC5(CCNCC5)CC6=O)c4c3)cn2)CC1] | 814 |
| 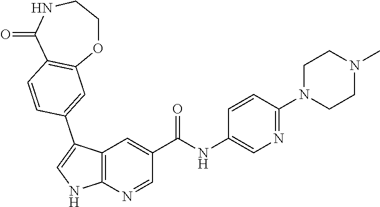 [SMILES: CN1CCN(c2ccc(NC(=O)c3cnc4[nH]cc(-c5ccc6c(c5)OCCNC6=O)c4c3)cn2)CC1] | 815 |
| 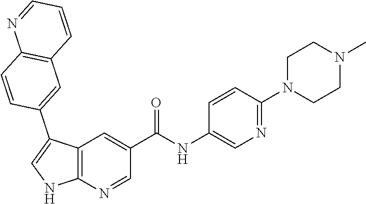 [SMILES: CN1CCN(c2ccc(NC(=O)c3cnc4[nH]cc(-c5ccc6ncccc6c5)c4c3)cn2)CC1] | 816 |
| 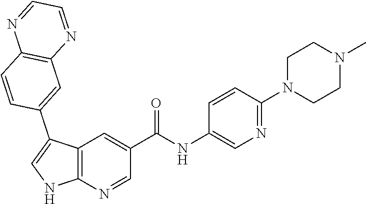 [SMILES: CN1CCN(c2ccc(NC(=O)c3cnc4[nH]cc(-c5ccc6nccnc6c5)c4c3)cn2)CC1] | 817 |
| 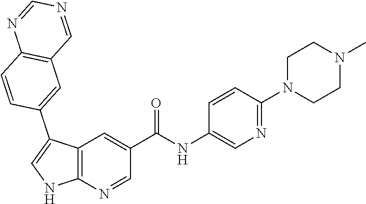 [SMILES: CN1CCN(c2ccc(NC(=O)c3cnc4[nH]cc(-c5ccc6ncncc6c5)c4c3)cn2)CC1] | 818 |
| 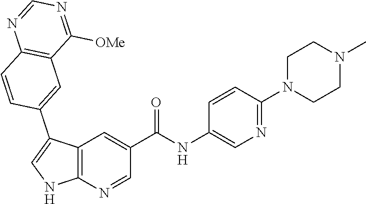 [SMILES: COc1ncnc2ccc(-c3c[nH]c4ncc(C(=O)Nc5ccc(N6CCN(C)CC6)nc5)cc34)cc12] | 819 |
| 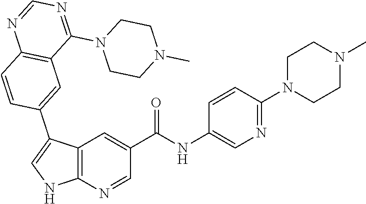 [SMILES: CN1CCN(c2ccc(NC(=O)c3cnc4[nH]cc(-c5ccc6ncnc(N7CCN(C)CC7)c6c5)c4c3)cn2)CC1] | 820 |
| 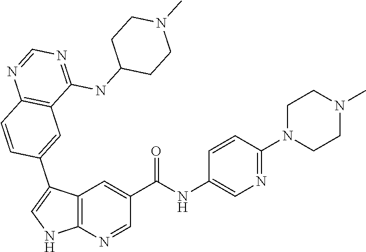 [SMILES: CN1CCC(Nc2ncnc3ccc(-c4c[nH]c5ncc(C(=O)Nc6ccc(N7CCN(C)CC7)nc6)cc45)cc23)CC1] | 821 |
| 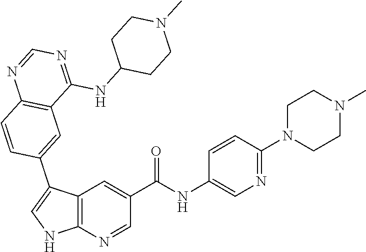 [SMILES: CN1CCC(Nc2ncnc3ccc(-c4c[nH]c5ncc(C(=O)Nc6ccc(N7CCN(C)CC7)nc6)cc45)cc23)CC1] | 822 |
| 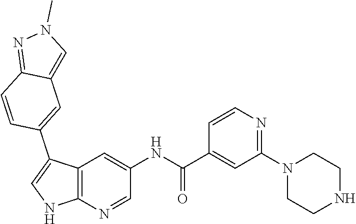 [SMILES: Cn1cc2cc(-c3c[nH]c4ncc(NC(=O)c5ccnc(N6CCNCC6)c5)cc34)ccc2n1] | 823 |
| 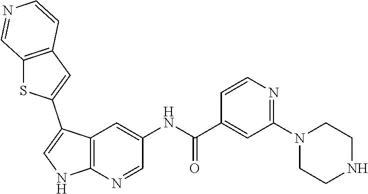 [SMILES: O=C(Nc1cnc2[nH]cc(-c3cc4ccncc4s3)c2c1)c1ccnc(N2CCNCC2)c1] | 824 |
| 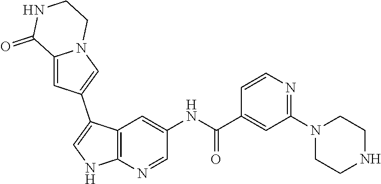 [SMILES: O=C(Nc1cnc2[nH]cc(-c3cc4n(c3)CCNC4=O)c2c1)c1ccnc(N2CCNCC2)c1] | 825 |
| 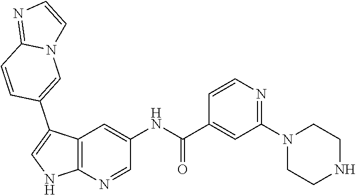 [SMILES: O=C(Nc1cnc2[nH]cc(-c3ccc4nccn4c3)c2c1)c1ccnc(N2CCNCC2)c1] | 826 |
| 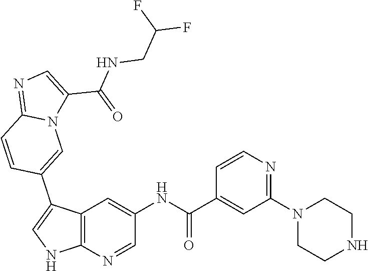 [SMILES: O=C(Nc1cnc2[nH]cc(-c3ccc4ncc(C(=O)NCC(F)F)n4c3)c2c1)c1ccnc(N2CCNCC2)c1] | 827 |
| 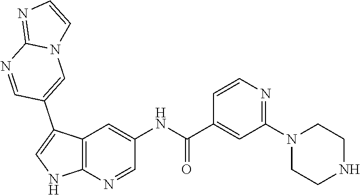 [SMILES: O=C(Nc1cnc2[nH]cc(-c3cnc4nccn4c3)c2c1)c1ccnc(N2CCNCC2)c1] | 828 |
| 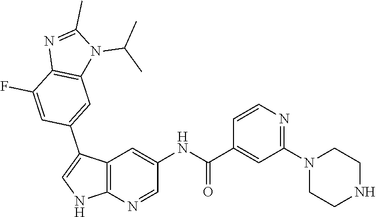 [SMILES: Cc1nc2c(F)cc(-c3c[nH]c4ncc(NC(=O)c5ccnc(N6CCNCC6)c5)cc34)cc2n1C(C)C] | 829 |
| 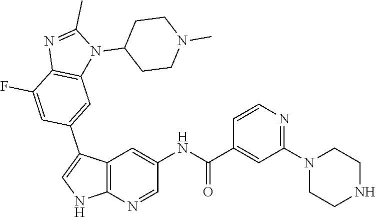 [SMILES: Cc1nc2c(F)cc(-c3c[nH]c4ncc(NC(=O)c5ccnc(N6CCNCC6)c5)cc34)cc2n1C1CCN(C)CC1] | 830 |
| 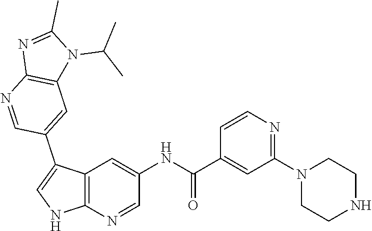 [SMILES: Cc1nc2ncc(-c3c[nH]c4ncc(NC(=O)c5ccnc(N6CCNCC6)c5)cc34)cc2n1C(C)C] | 831 |
| 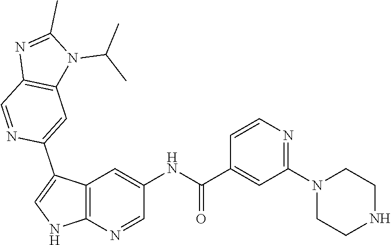 [SMILES: Cc1nc2cnc(-c3c[nH]c4ncc(NC(=O)c5ccnc(N6CCNCC6)c5)cc34)cc2n1C(C)C] | 832 |
| 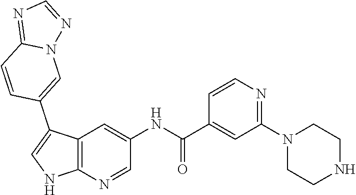 [SMILES: O=C(Nc1cnc2[nH]cc(-c3ccc4ncnn4c3)c2c1)c1ccnc(N2CCNCC2)c1] | 833 |
| 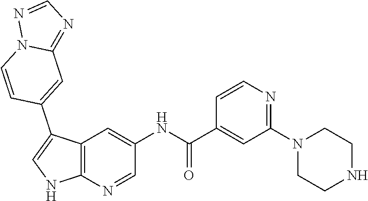 [SMILES: O=C(Nc1cnc2[nH]cc(-c3ccn4ncnc4c3)c2c1)c1ccnc(N2CCNCC2)c1] | 834 |
| 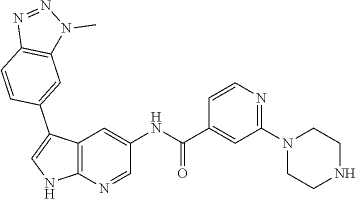 [SMILES: Cn1nnc2ccc(-c3c[nH]c4ncc(NC(=O)c5ccnc(N6CCNCC6)c5)cc34)cc21] | 835 |
| 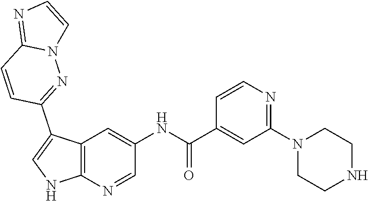 [SMILES: O=C(Nc1cnc2[nH]cc(-c3ccc4nccn4n3)c2c1)c1ccnc(N2CCNCC2)c1] | 836 |
| 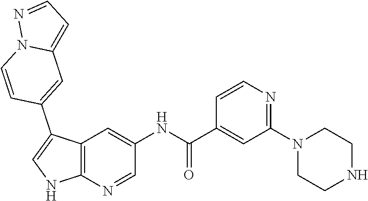 [SMILES: O=C(Nc1cnc2[nH]cc(-c3ccn4nccc4c3)c2c1)c1ccnc(N2CCNCC2)c1] | 837 |
| 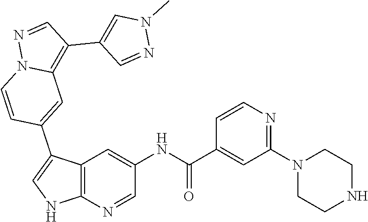 [SMILES: Cn1cc(-c2cnn3ccc(-c4c[nH]c5ncc(NC(=O)c6ccnc(N7CCNCC7)c6)cc45)cc23)cn1] | 838 |
| 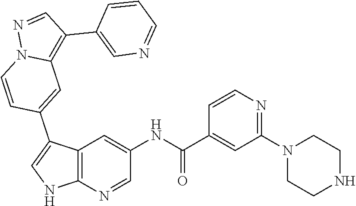 [SMILES: O=C(Nc1cnc2[nH]cc(-c3ccn4ncc(-c5cccnc5)c4c3)c2c1)c1ccnc(N2CCNCC2)c1] | 839 |
| 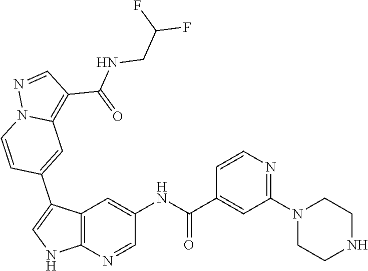 [SMILES: O=C(Nc1cnc2[nH]cc(-c3ccn4ncc(C(=O)NCC(F)F)c4c3)c2c1)c1ccnc(N2CCNCC2)c1] | 840 |
| 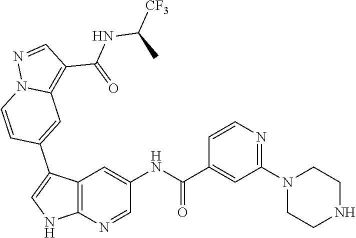 [SMILES: C[C@@H](NC(=O)c1cnn2ccc(-c3c[nH]c4ncc(NC(=O)c5ccnc(N6CCNCC6)c5)cc34)cc12)C(F)(F)F] | 841 |
| 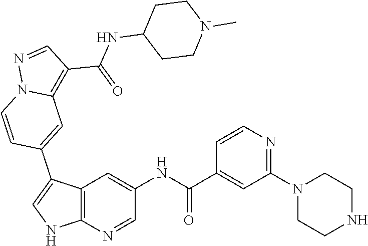 [SMILES: CN1CCC(NC(=O)c2cnn3ccc(-c4c[nH]c5ncc(NC(=O)c6ccnc(N7CCNCC7)c6)cc45)cc23)CC1] | 842 |
| 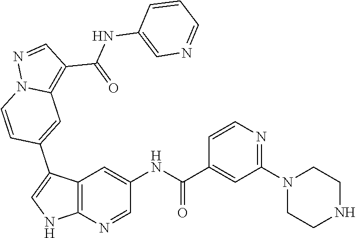 [SMILES: O=C(Nc1cnc2[nH]cc(-c3ccn4ncc(C(=O)Nc5cccnc5)c4c3)c2c1)c1ccnc(N2CCNCC2)c1] | 843 |
| 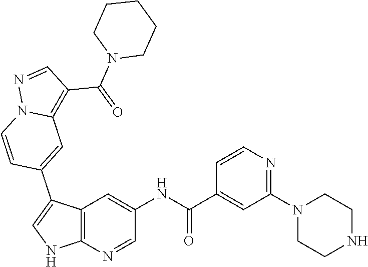 [SMILES: O=C(Nc1cnc2[nH]cc(-c3ccn4ncc(C(=O)N5CCCCC5)c4c3)c2c1)c1ccnc(N2CCNCC2)c1] | 844 |
| 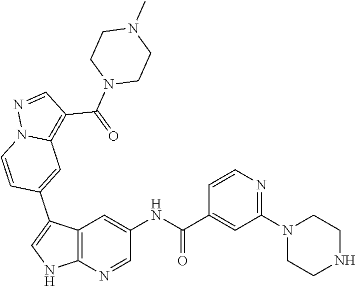 [SMILES: CN1CCN(C(=O)c2cnn3ccc(-c4c[nH]c5ncc(NC(=O)c6ccnc(N7CCNCC7)c6)cc45)cc23)CC1] | 845 |
| 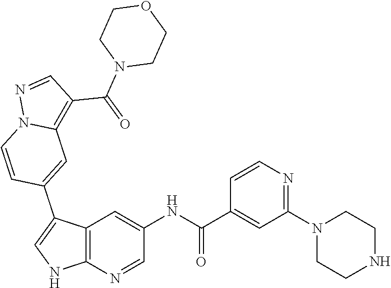 [SMILES: O=C(Nc1cnc2[nH]cc(-c3ccn4ncc(C(=O)N5CCOCC5)c4c3)c2c1)c1ccnc(N2CCNCC2)c1] | 846 |
| 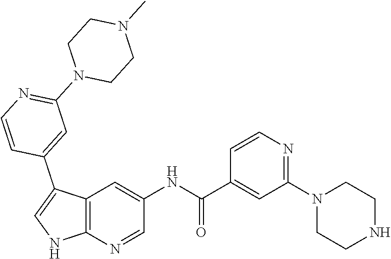 [SMILES: CN1CCN(c2cc(-c3c[nH]c4ncc(NC(=O)c5ccnc(N6CCNCC6)c5)cc34)ccn2)CC1] | 847 |
| 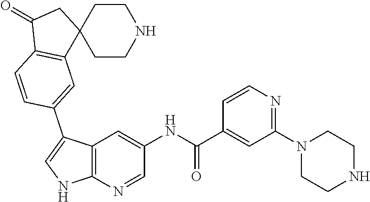 [SMILES: O=C(Nc1cnc2[nH]cc(-c3ccc4c(c3)C3(CCNCC3)CC4=O)c2c1)c1ccnc(N2CCNCC2)c1] | 848 |
| 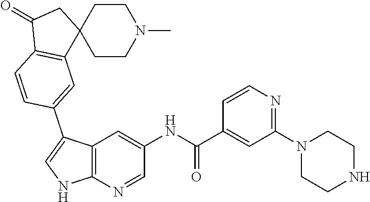 [SMILES: CN1CCC2(CC1)CC(=O)c1ccc(-c3c[nH]c4ncc(NC(=O)c5ccnc(N6CCNCC6)c5)cc34)cc12] | 849 |
| 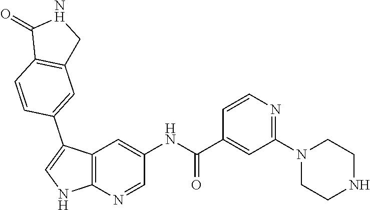 [SMILES: O=C(Nc1cnc2[nH]cc(-c3ccc4c(c3)CNC4=O)c2c1)c1ccnc(N2CCNCC2)c1] | 850 |
| 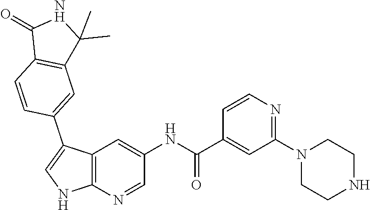 [SMILES: CC1(C)NC(=O)c2ccc(-c3c[nH]c4ncc(NC(=O)c5ccnc(N6CCNCC6)c5)cc34)cc21] | 851 |
| 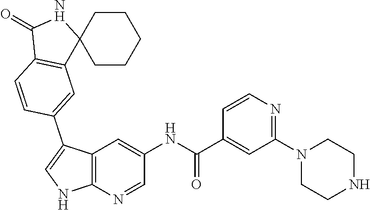 [SMILES: O=C(Nc1cnc2[nH]cc(-c3ccc4c(c3)C3(CCCCC3)NC4=O)c2c1)c1ccnc(N2CCNCC2)c1] | 852 |
| 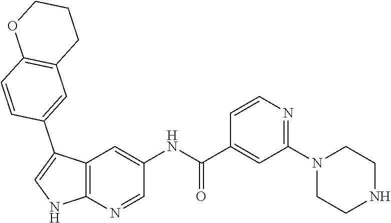 [SMILES: O=C(Nc1cnc2[nH]cc(-c3ccc4c(c3)CCCO4)c2c1)c1ccnc(N2CCNCC2)c1] | 853 |
| 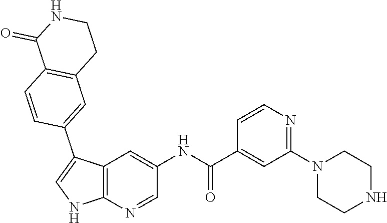 [SMILES: O=C(Nc1cnc2[nH]cc(-c3ccc4c(c3)CCNC4=O)c2c1)c1ccnc(N2CCNCC2)c1] | 854 |
| 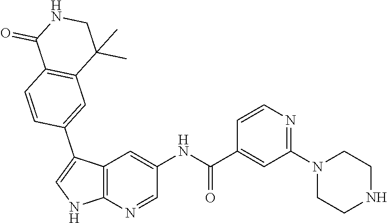 [SMILES: CC1(C)CNC(=O)c2ccc(-c3c[nH]c4ncc(NC(=O)c5ccnc(N6CCNCC6)c5)cc34)cc21] | 855 |
| 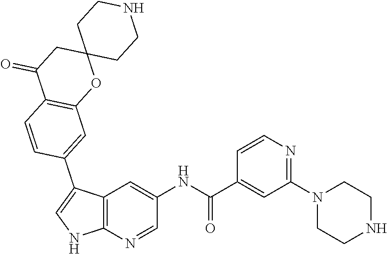 [SMILES: O=C(Nc1cnc2[nH]cc(-c3ccc4c(c3)OC3(CCNCC3)CC4=O)c2c1)c1ccnc(N2CCNCC2)c1] | 856 |
| 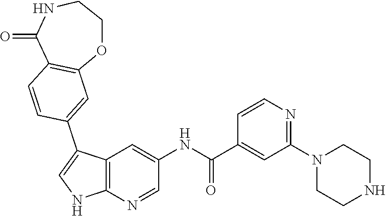 [SMILES: O=C(Nc1cnc2[nH]cc(-c3ccc4c(c3)OCCNC4=O)c2c1)c1ccnc(N2CCNCC2)c1] | 857 |
| 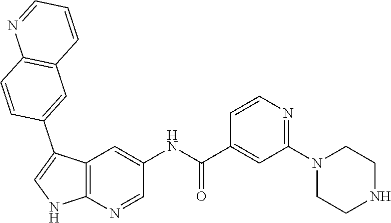 [SMILES: O=C(Nc1cnc2[nH]cc(-c3ccc4ncccc4c3)c2c1)c1ccnc(N2CCNCC2)c1] | 858 |
| 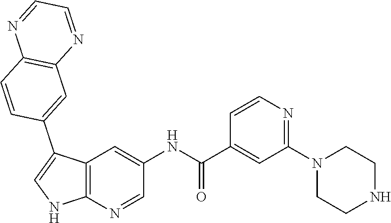 [SMILES: O=C(Nc1cnc2[nH]cc(-c3ccc4nccnc4c3)c2c1)c1ccnc(N2CCNCC2)c1] | 859 |
| 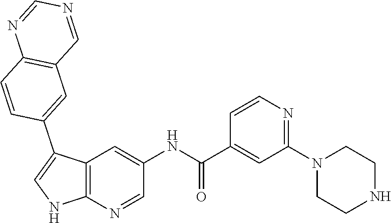 [SMILES: O=C(Nc1cnc2[nH]cc(-c3ccc4ncncc4c3)c2c1)c1ccnc(N2CCNCC2)c1] | 860 |
| 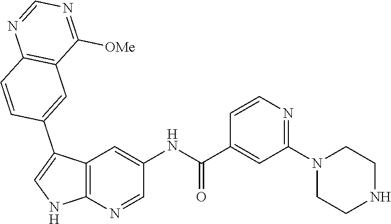 [SMILES: COc1ncnc2ccc(-c3c[nH]c4ncc(NC(=O)c5ccnc(N6CCNCC6)c5)cc34)cc12] | 861 |
| 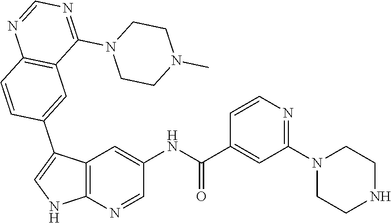 [SMILES: CN1CCN(c2ncnc3ccc(-c4c[nH]c5ncc(NC(=O)c6ccnc(N7CCNCC7)c6)cc45)cc23)CC1] | 862 |
| 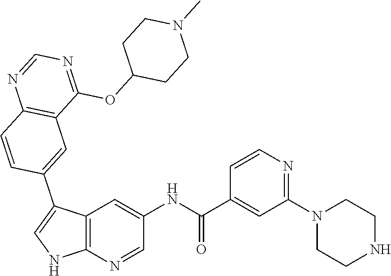 [SMILES: CN1CCC(Oc2ncnc3ccc(-c4c[nH]c5ncc(NC(=O)c6ccnc(N7CCNCC7)c6)cc45)cc23)CC1] | 863 |
| 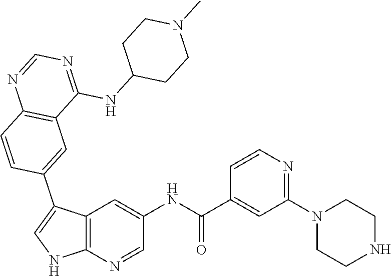 [SMILES: CN1CCC(Nc2ncnc3ccc(-c4c[nH]c5ncc(NC(=O)c6ccnc(N7CCNCC7)c6)cc45)cc23)CC1] | 864 |
| 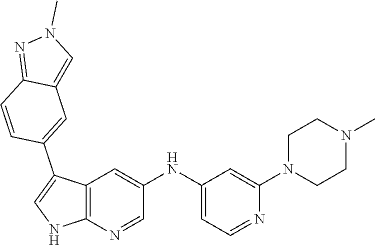 [SMILES: CN1CCN(c2cc(Nc3cnc4[nH]cc(-c5ccc6nn(C)cc6c5)c4c3)ccn2)CC1] | 865 |
| 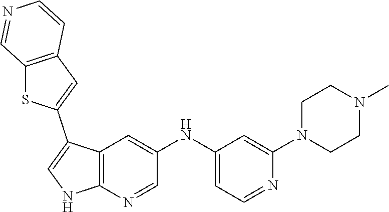 [SMILES: CN1CCN(c2cc(Nc3cnc4[nH]cc(-c5cc6ccncc6s5)c4c3)ccn2)CC1] | 866 |
| 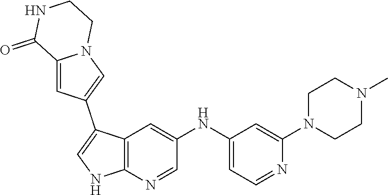 [SMILES: CN1CCN(c2cc(Nc3cnc4[nH]cc(-c5cc6n(c5)CCNC6=O)c4c3)ccn2)CC1] | 867 |
| 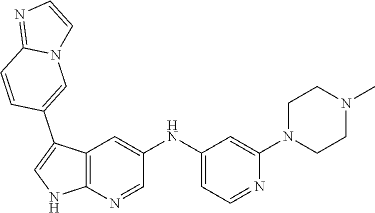 [SMILES: CN1CCN(c2cc(Nc3cnc4[nH]cc(-c5ccc6nccn6c5)c4c3)ccn2)CC1] | 868 |
| 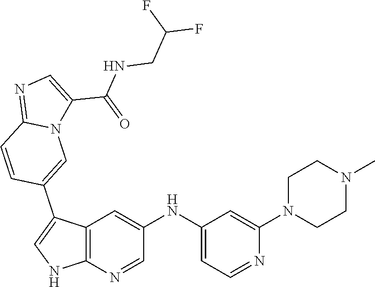 [SMILES: CN1CCN(c2cc(Nc3cnc4[nH]cc(-c5ccc6ncc(C(=O)NCC(F)F)n6c5)c4c3)ccn2)CC1] | 869 |
| 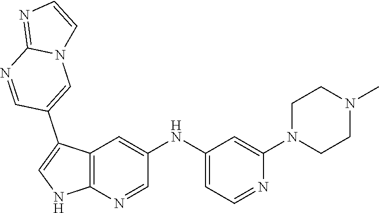 [SMILES: CN1CCN(c2cc(Nc3cnc4[nH]cc(-c5cnc6nccn6c5)c4c3)ccn2)CC1] | 870 |
| 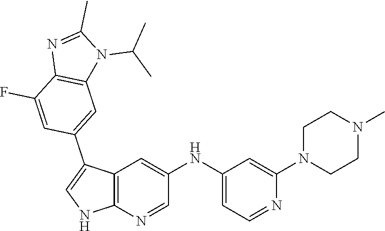 [SMILES: Cc1nc2c(F)cc(-c3c[nH]c4ncc(Nc5ccnc(N6CCN(C)CC6)c5)cc34)cc2n1C(C)C] | 871 |
| 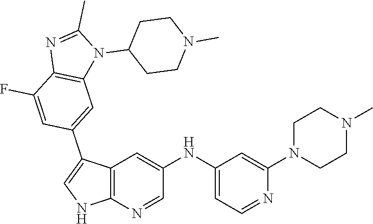 [SMILES: Cc1nc2c(F)cc(-c3c[nH]c4ncc(Nc5ccnc(N6CCN(C)CC6)c5)cc34)cc2n1C1CCN(C)CC1] | 872 |
| 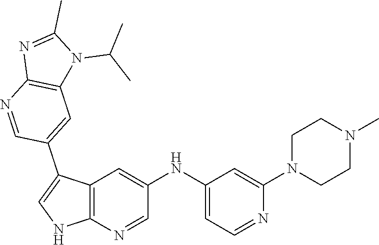 [SMILES: Cc1nc2ncc(-c3c[nH]c4ncc(Nc5ccnc(N6CCN(C)CC6)c5)cc34)cc2n1C(C)C] | 873 |
| 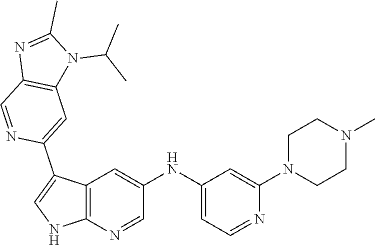 [SMILES: Cc1nc2cnc(-c3c[nH]c4ncc(Nc5ccnc(N6CCN(C)CC6)c5)cc34)cc2n1C(C)C] | 874 |
| 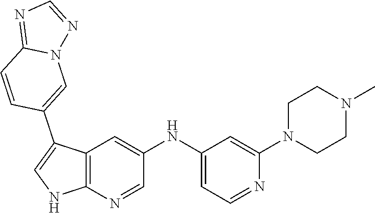 [SMILES: CN1CCN(c2cc(Nc3cnc4[nH]cc(-c5ccc6ncnn6c5)c4c3)ccn2)CC1] | 875 |
| 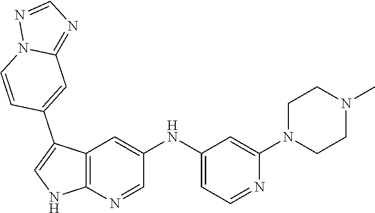 [SMILES: CN1CCN(c2cc(Nc3cnc4[nH]cc(-c5ccn6ncnc6c5)c4c3)ccn2)CC1] | 876 |
| 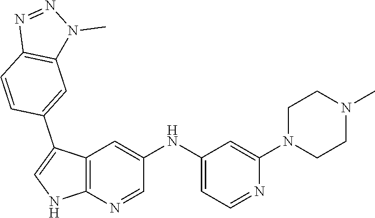 [SMILES: CN1CCN(c2cc(Nc3cnc4[nH]cc(-c5ccc6nnn(C)c6c5)c4c3)ccn2)CC1] | 877 |
| 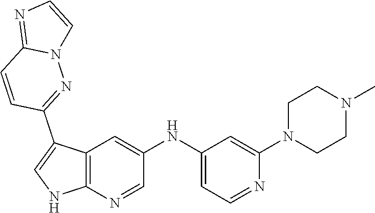 [SMILES: CN1CCN(c2cc(Nc3cnc4[nH]cc(-c5ccc6nccn6n5)c4c3)ccn2)CC1] | 878 |
| 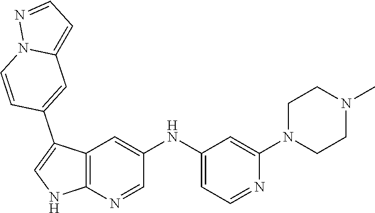 [SMILES: CN1CCN(c2cc(Nc3cnc4[nH]cc(-c5ccn6nccc6c5)c4c3)ccn2)CC1] | 879 |
| 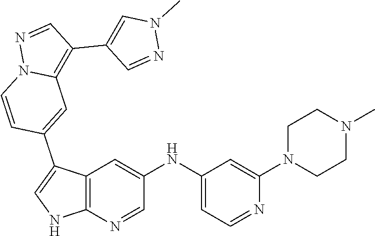 [SMILES: CN1CCN(c2cc(Nc3cnc4[nH]cc(-c5ccn6ncc(-c7cnn(C)c7)c6c5)c4c3)ccn2)CC1] | 880 |
| 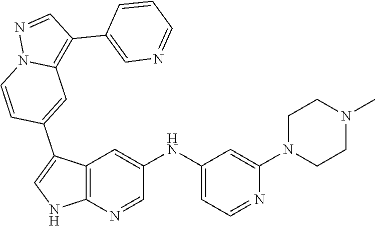 [SMILES: CN1CCN(c2cc(Nc3cnc4[nH]cc(-c5ccn6ncc(-c7cccnc7)c6c5)c4c3)ccn2)CC1] | 881 |
| 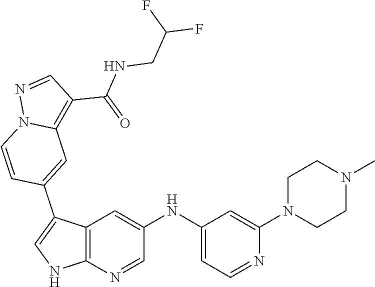 [SMILES: CN1CCN(c2cc(Nc3cnc4[nH]cc(-c5ccn6ncc(C(=O)NCC(F)F)c6c5)c4c3)ccn2)CC1] | 882 |
| 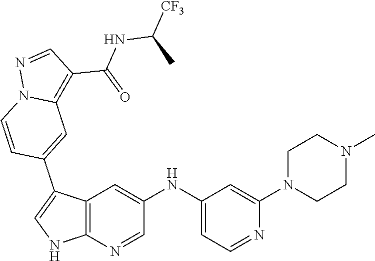 [SMILES: C[C@@H](NC(=O)c1cnn2ccc(-c3c[nH]c4ncc(Nc5ccnc(N6CCN(C)CC6)c5)cc34)cc12)C(F)(F)F] | 883 |
| 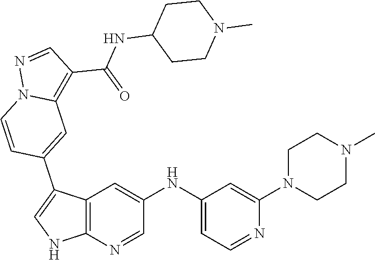 [SMILES: CN1CCC(NC(=O)c2cnn3ccc(-c4c[nH]c5ncc(Nc6ccnc(N7CCN(C)CC7)c6)cc45)cc23)CC1] | 884 |
| 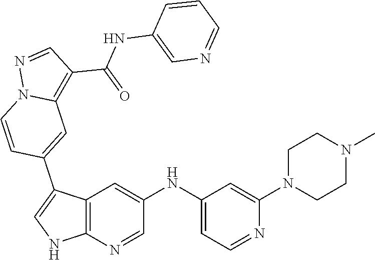 [SMILES: CN1CCN(c2cc(Nc3cnc4[nH]cc(-c5ccn6ncc(C(=O)Nc7cccnc7)c6c5)c4c3)ccn2)CC1] | 885 |
| 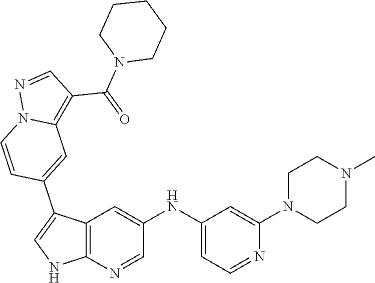 [SMILES: CN1CCN(c2cc(Nc3cnc4[nH]cc(-c5ccn6ncc(C(=O)N7CCCCC7)c6c5)c4c3)ccn2)CC1] | 886 |
| 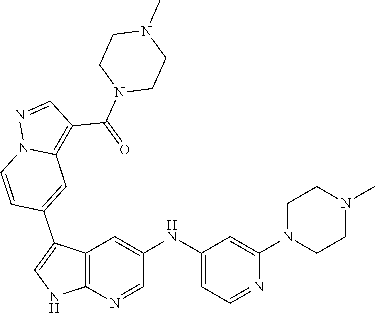 [SMILES: CN1CCN(C(=O)c2cnn3ccc(-c4c[nH]c5ncc(Nc6ccnc(N7CCN(C)CC7)c6)cc45)cc23)CC1] | 887 |
| 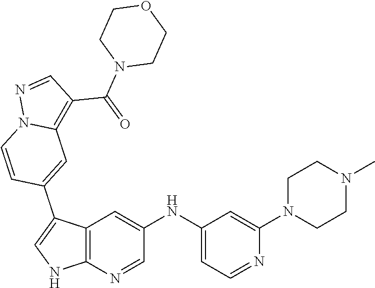 [SMILES: CN1CCN(c2cc(Nc3cnc4[nH]cc(-c5ccn6ncc(C(=O)N7CCOCC7)c6c5)c4c3)ccn2)CC1] | 888 |
| 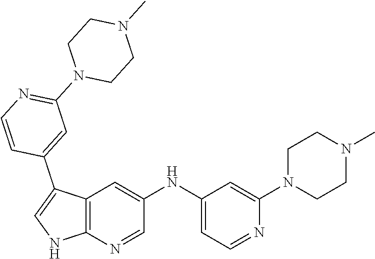 [SMILES: CN1CCN(c2cc(Nc3cnc4[nH]cc(-c5ccnc(N6CCN(C)CC6)c5)c4c3)ccn2)CC1] | 889 |
| 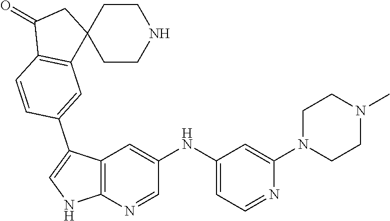 [SMILES: CN1CCN(c2cc(Nc3cnc4[nH]cc(-c5ccc6c(c5)C5(CCNCC5)CC6=O)c4c3)ccn2)CC1] | 890 |
| 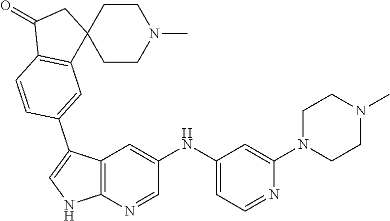 [SMILES: CN1CCN(c2cc(Nc3cnc4[nH]cc(-c5ccc6c(c5)C5(CCN(C)CC5)CC6=O)c4c3)ccn2)CC1] | 891 |
| 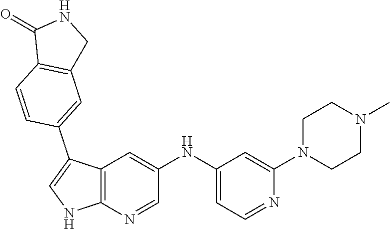 [SMILES: CN1CCN(c2cc(Nc3cnc4[nH]cc(-c5ccc6c(c5)CNC6=O)c4c3)ccn2)CC1] | 892 |
| 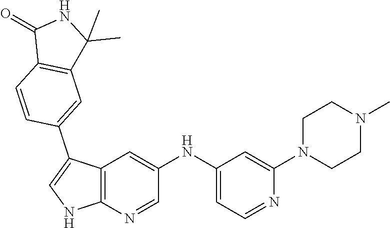 [SMILES: CN1CCN(c2cc(Nc3cnc4[nH]cc(-c5ccc6c(c5)C(C)(C)NC6=O)c4c3)ccn2)CC1] | 893 |
| 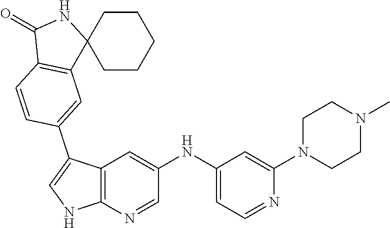 [SMILES: CN1CCN(c2cc(Nc3cnc4[nH]cc(-c5ccc6c(c5)C5(CCCCC5)NC6=O)c4c3)ccn2)CC1] | 894 |
| 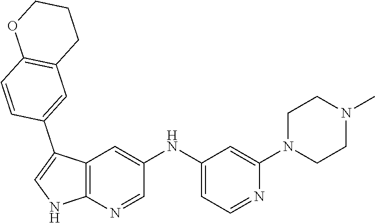 [SMILES: CN1CCN(c2cc(Nc3cnc4[nH]cc(-c5ccc6c(c5)CCCO6)c4c3)ccn2)CC1] | 895 |
| 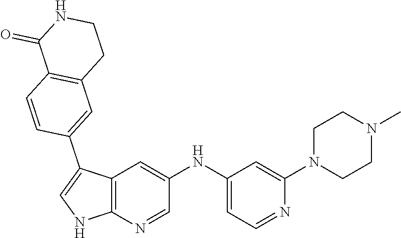 [SMILES: CN1CCN(c2cc(Nc3cnc4[nH]cc(-c5ccc6c(c5)CCNC6=O)c4c3)ccn2)CC1] | 896 |
| 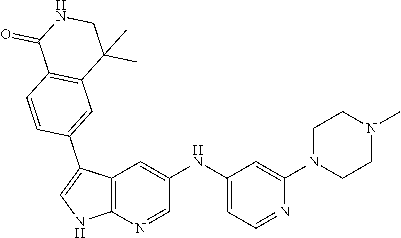 [SMILES: CN1CCN(c2cc(Nc3cnc4[nH]cc(-c5ccc6c(c5)C(C)(C)CNC6=O)c4c3)ccn2)CC1] | 897 |
| 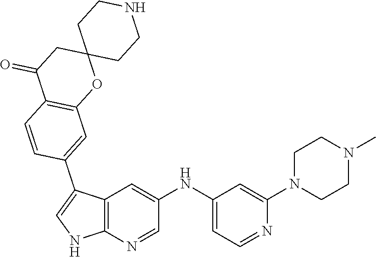 [SMILES: CN1CCN(c2cc(Nc3cnc4[nH]cc(-c5ccc6c(c5)OC5(CCNCC5)CC6=O)c4c3)ccn2)CC1] | 898 |
| 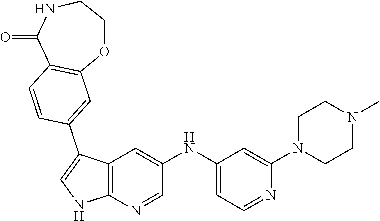 [SMILES: CN1CCN(c2cc(Nc3cnc4[nH]cc(-c5ccc6c(c5)OCCNC6=O)c4c3)ccn2)CC1] | 899 |
| 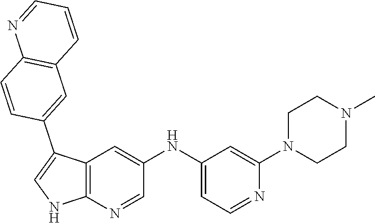 [SMILES: CN1CCN(c2cc(Nc3cnc4[nH]cc(-c5ccc6ncccc6c5)c4c3)ccn2)CC1] | 900 |
| 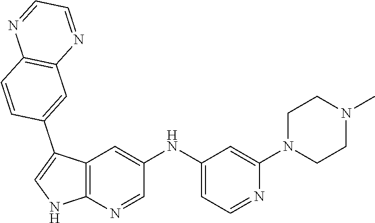 [SMILES: CN1CCN(c2cc(Nc3cnc4[nH]cc(-c5ccc6nccnc6c5)c4c3)ccn2)CC1] | 901 |
| 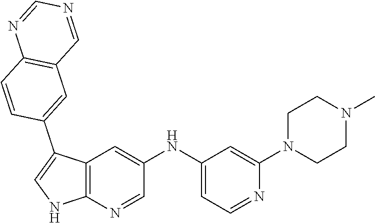 [SMILES: CN1CCN(c2cc(Nc3cnc4[nH]cc(-c5ccc6ncncc6c5)c4c3)ccn2)CC1] | 902 |
| 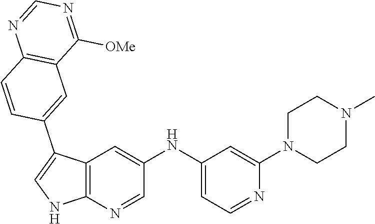 [SMILES: COc1ncnc2ccc(-c3c[nH]c4ncc(Nc5ccnc(N6CCN(C)CC6)c5)cc34)cc12] | 903 |
| 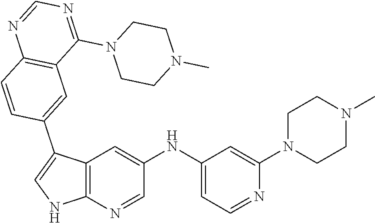 [SMILES: CN1CCN(c2cc(Nc3cnc4[nH]cc(-c5ccc6ncnc(N7CCN(C)CC7)c6c5)c4c3)ccn2)CC1] | 904 |
| 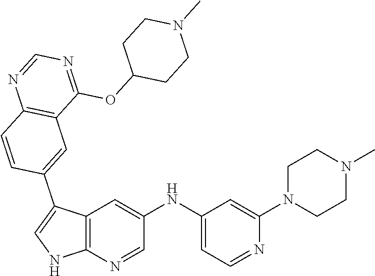 [SMILES: CN1CCC(Oc2ncnc3ccc(-c4c[nH]c5ncc(Nc6ccnc(N7CCN(C)CC7)c6)cc45)cc23)CC1] | 905 |
| 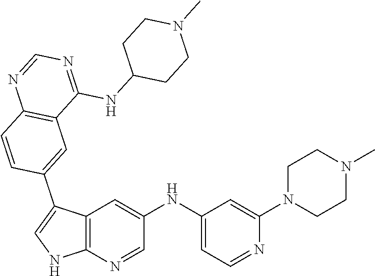 [SMILES: CN1CCC(Nc2ncnc3ccc(-c4c[nH]c5ncc(Nc6ccnc(N7CCN(C)CC7)c6)cc45)cc23)CC1] | 906 |
| 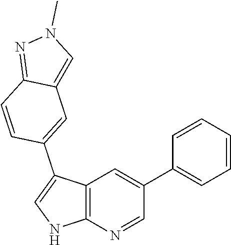 [SMILES: Cn1cc2cc(-c3c[nH]c4ncc(-c5ccccc5)cc34)ccc2n1] | 907 |
| 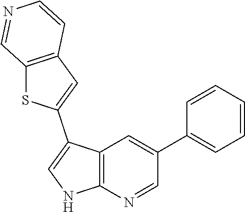 [SMILES: c1ccc(-c2cnc3[nH]cc(-c4cc5ccncc5s4)c3c2)cc1] | 908 |
| 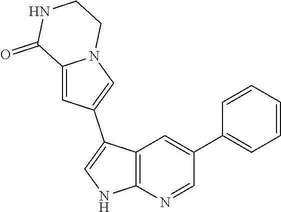 [SMILES: O=C1NCCn2cc(-c3c[nH]c4ncc(-c5ccccc5)cc34)cc21] | 909 |
| 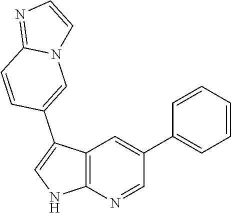 [SMILES: c1ccc(-c2cnc3[nH]cc(-c4ccc5nccn5c4)c3c2)cc1] | 910 |
| 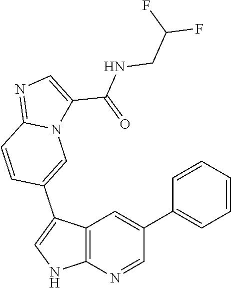 [SMILES: O=C(NCC(F)F)c1cnc2ccc(-c3c[nH]c4ncc(-c5ccccc5)cc34)cn12] | 911 |
| 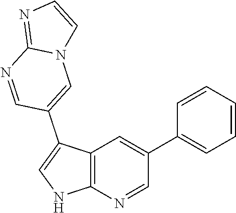 [SMILES: c1ccc(-c2cnc3[nH]cc(-c4cnc5nccn5c4)c3c2)cc1] | 912 |
| 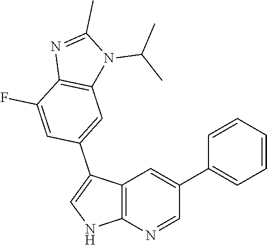 [SMILES: Cc1nc2c(F)cc(-c3c[nH]c4ncc(-c5ccccc5)cc34)cc2n1C(C)C] | 913 |
| 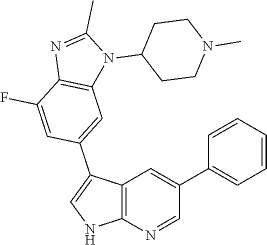 [SMILES: Cc1nc2c(F)cc(-c3c[nH]c4ncc(-c5ccccc5)cc34)cc2n1C1CCN(C)CC1] | 914 |
| 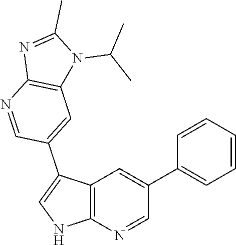 [SMILES: Cc1nc2ncc(-c3c[nH]c4ncc(-c5ccccc5)cc34)cc2n1C(C)C] | 915 |
| 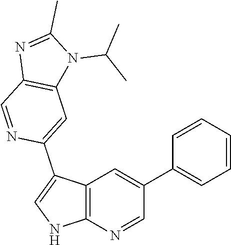 [SMILES: Cc1nc2cnc(-c3c[nH]c4ncc(-c5ccccc5)cc34)cc2n1C(C)C] | 916 |
| 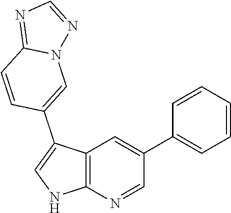 [SMILES: c1ccc(-c2cnc3[nH]cc(-c4ccc5ncnn5c4)c3c2)cc1] | 917 |
| 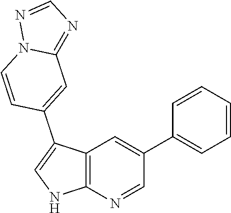 [SMILES: c1ccc(-c2cnc3[nH]cc(-c4ccn5ncnc5c4)c3c2)cc1] | 918 |
| 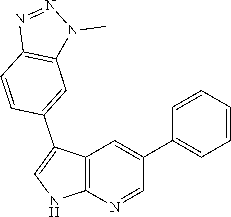 [SMILES: Cn1nnc2ccc(-c3c[nH]c4ncc(-c5ccccc5)cc34)cc21] | 919 |
| 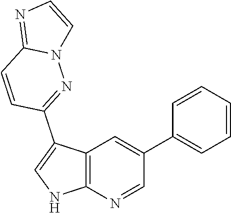 [SMILES: c1ccc(-c2cnc3[nH]cc(-c4ccc5nccn5n4)c3c2)cc1] | 920 |
| 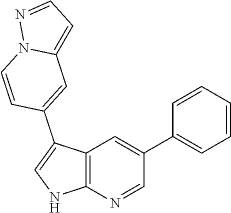 [SMILES: c1ccc(-c2cnc3[nH]cc(-c4ccn5nccc5c4)c3c2)cc1] | 921 |
| 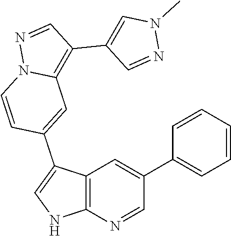 [SMILES: Cn1cc(-c2cnn3ccc(-c4c[nH]c5ncc(-c6ccccc6)cc45)cc23)cn1] | 922 |
| 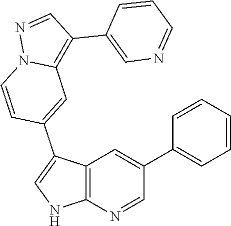 [SMILES: c1ccc(-c2cnc3[nH]cc(-c4ccn5ncc(-c6cccnc6)c5c4)c3c2)cc1] | 923 |
| 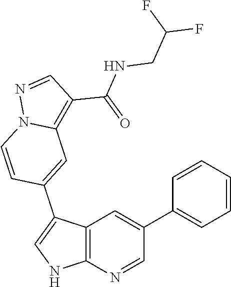 [SMILES: O=C(NCC(F)F)c1cnn2ccc(-c3c[nH]c4ncc(-c5ccccc5)cc34)cc12] | 924 |
| 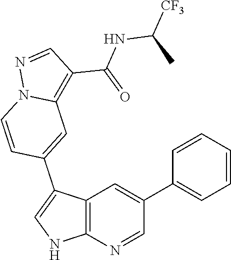 [SMILES: C[C@@H](NC(=O)c1cnn2ccc(-c3c[nH]c4ncc(-c5ccccc5)cc34)cc12)C(F)(F)F] | 925 |
| 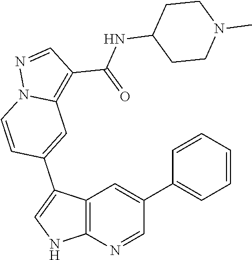 [SMILES: CN1CCC(NC(=O)c2cnn3ccc(-c4c[nH]c5ncc(-c6ccccc6)cc45)cc23)CC1] | 926 |
| 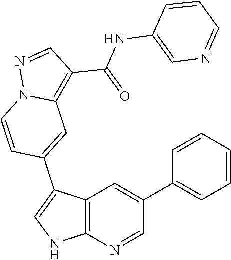 [SMILES: O=C(Nc1cccnc1)c1cnn2ccc(-c3c[nH]c4ncc(-c5ccccc5)cc34)cc12] | 927 |
| 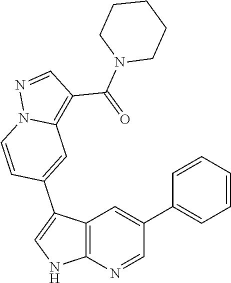 [SMILES: O=C(c1cnn2ccc(-c3c[nH]c4ncc(-c5ccccc5)cc34)cc12)N1CCCCC1] | 928 |
| 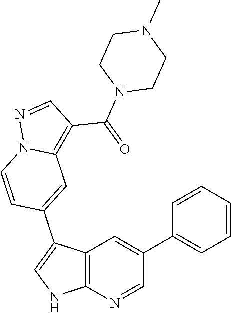 [SMILES: CN1CCN(C(=O)c2cnn3ccc(-c4c[nH]c5ncc(-c6ccccc6)cc45)cc23)CC1] | 929 |
| 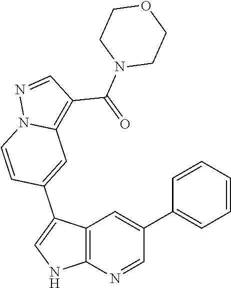 [SMILES: O=C(c1cnn2ccc(-c3c[nH]c4ncc(-c5ccccc5)cc34)cc12)N1CCOCC1] | 930 |
| 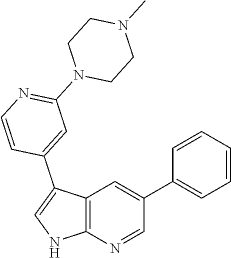 [SMILES: CN1CCN(c2cc(-c3c[nH]c4ncc(-c5ccccc5)cc34)ccn2)CC1] | 931 |
| 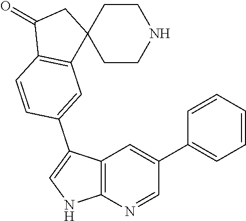 [SMILES: O=C1CC2(CCNCC2)c2cc(-c3c[nH]c4ncc(-c5ccccc5)cc34)ccc21] | 932 |
| 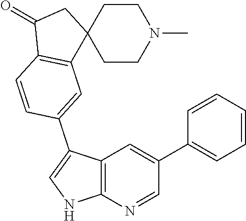 [SMILES: CN1CCC2(CC1)CC(=O)c1ccc(-c3c[nH]c4ncc(-c5ccccc5)cc34)cc12] | 933 |
| 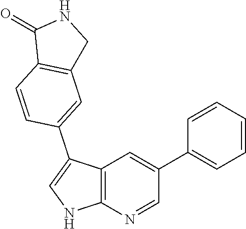 [SMILES: O=C1NCc2cc(-c3c[nH]c4ncc(-c5ccccc5)cc34)ccc21] | 934 |
| 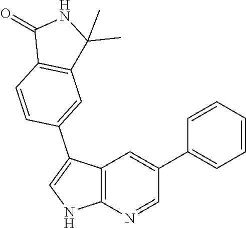 [SMILES: CC1(C)NC(=O)c2ccc(-c3c[nH]c4ncc(-c5ccccc5)cc34)cc21] | 935 |
| 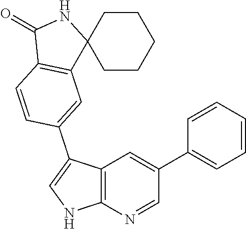 [SMILES: O=C1NC2(CCCCC2)c2cc(-c3c[nH]c4ncc(-c5ccccc5)cc34)ccc21] | 936 |
| 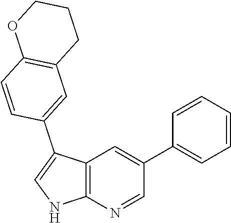 [SMILES: c1ccc(-c2cnc3[nH]cc(-c4ccc5c(c4)CCCO5)c3c2)cc1] | 937 |
| 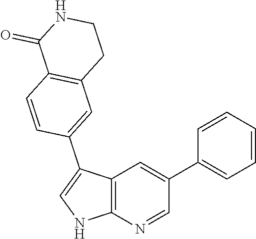 [SMILES: O=C1NCCc2cc(-c3c[nH]c4ncc(-c5ccccc5)cc34)ccc21] | 938 |
| 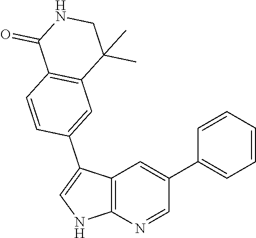 [SMILES: CC1(C)CNC(=O)c2ccc(-c3c[nH]c4ncc(-c5ccccc5)cc34)cc21] | 939 |
| 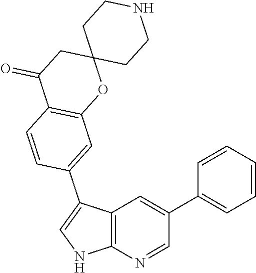 [SMILES: O=C1CC2(CCNCC2)Oc2cc(-c3c[nH]c4ncc(-c5ccccc5)cc34)ccc21] | 940 |
| 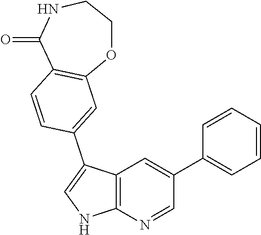 [SMILES: O=C1NCCOc2cc(-c3c[nH]c4ncc(-c5ccccc5)cc34)ccc21] | 941 |
| 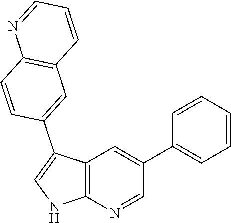 [SMILES: c1ccc(-c2cnc3[nH]cc(-c4ccc5ncccc5c4)c3c2)cc1] | 942 |
| 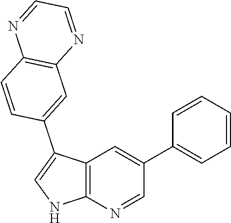 [SMILES: c1ccc(-c2cnc3[nH]cc(-c4ccc5nccnc5c4)c3c2)cc1] | 943 |
| 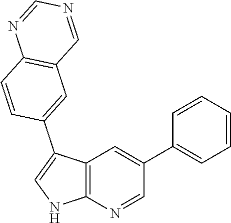 [SMILES: c1ccc(-c2cnc3[nH]cc(-c4ccc5ncncc5c4)c3c2)cc1] | 944 |
| 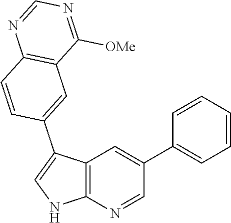 [SMILES: COc1ncnc2ccc(-c3c[nH]c4ncc(-c5ccccc5)cc34)cc12] | 945 |
| 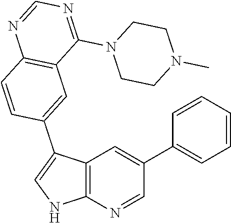 [SMILES: CN1CCN(c2ncnc3ccc(-c4c[nH]c5ncc(-c6ccccc6)cc45)cc23)CC1] | 946 |
| 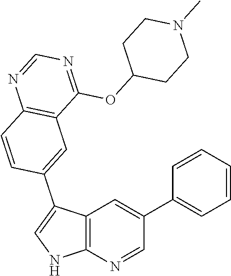 [SMILES: CN1CCC(Oc2ncnc3ccc(-c4c[nH]c5ncc(-c6ccccc6)cc45)cc23)CC1] | 947 |
| 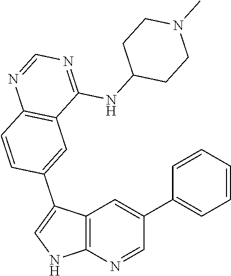 [SMILES: CN1CCC(Nc2ncnc3ccc(-c4c[nH]c5ncc(-c6ccccc6)cc45)cc23)CC1] | 948 |
| 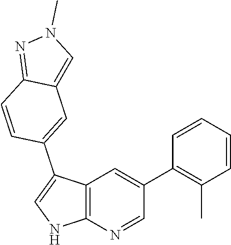 [SMILES: Cc1ccccc1-c1cnc2[nH]cc(-c3ccc4nn(C)cc4c3)c2c1] | 949 |
| 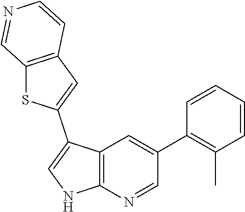 [SMILES: Cc1ccccc1-c1cnc2[nH]cc(-c3cc4ccncc4s3)c2c1] | 950 |
| 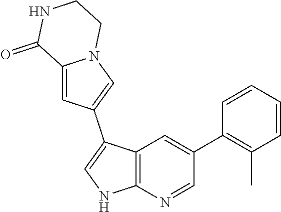 [SMILES: Cc1ccccc1-c1cnc2[nH]cc(-c3cc4n(c3)CCNC4=O)c2c1] | 951 |
| 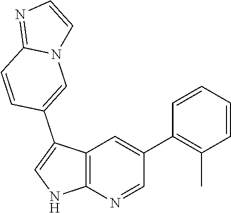 [SMILES: Cc1ccccc1-c1cnc2[nH]cc(-c3ccc4nccn4c3)c2c1] | 952 |
| 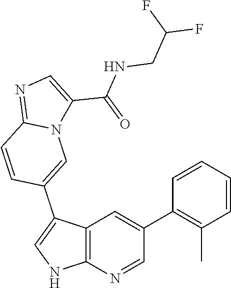 [SMILES: Cc1ccccc1-c1cnc2[nH]cc(-c3ccc4ncc(C(=O)NCC(F)F)n4c3)c2c1] | 953 |
| 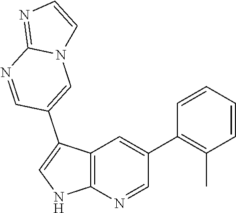 [SMILES: Cc1ccccc1-c1cnc2[nH]cc(-c3cnc4nccn4c3)c2c1] | 954 |
| 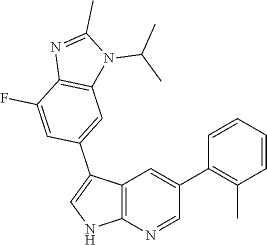 [SMILES: Cc1ccccc1-c1cnc2[nH]cc(-c3cc(F)c4nc(C)n(C(C)C)c4c3)c2c1] | 955 |
| 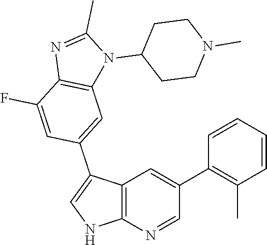 [SMILES: Cc1ccccc1-c1cnc2[nH]cc(-c3cc(F)c4nc(C)n(C5CCN(C)CC5)c4c3)c2c1] | 956 |
| 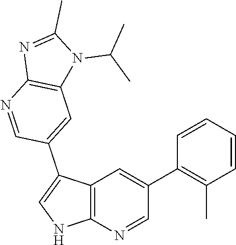 [SMILES: Cc1ccccc1-c1cnc2[nH]cc(-c3cnc4nc(C)n(C(C)C)c4c3)c2c1] | 957 |
| 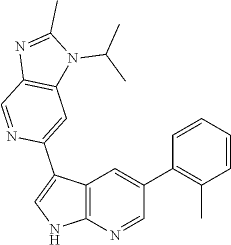 [SMILES: Cc1ccccc1-c1cnc2[nH]cc(-c3cc4c(cn3)nc(C)n4C(C)C)c2c1] | 958 |
| 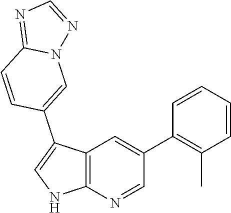 [SMILES: Cc1ccccc1-c1cnc2[nH]cc(-c3ccc4ncnn4c3)c2c1] | 959 |
| 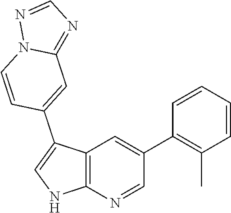 [SMILES: Cc1ccccc1-c1cnc2[nH]cc(-c3ccn4ncnc4c3)c2c1] | 960 |
| 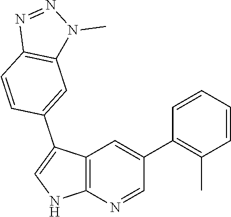 [SMILES: Cc1ccccc1-c1cnc2[nH]cc(-c3ccc4nnn(C)c4c3)c2c1] | 961 |
| 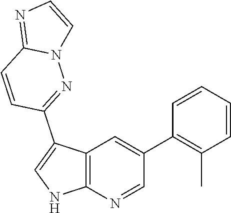 [SMILES: Cc1ccccc1-c1cnc2[nH]cc(-c3ccc4nccn4n3)c2c1] | 962 |
| 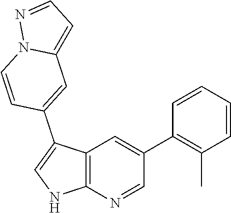 [SMILES: Cc1ccccc1-c1cnc2[nH]cc(-c3ccn4nccc4c3)c2c1] | 963 |
| 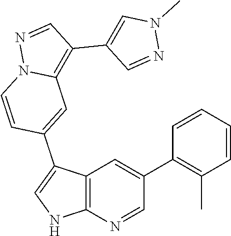 [SMILES: Cc1ccccc1-c1cnc2[nH]cc(-c3ccn4ncc(-c5cnn(C)c5)c4c3)c2c1] | 964 |
| 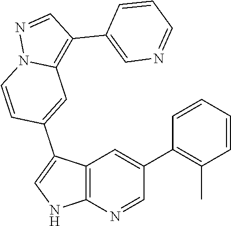 [SMILES: Cc1ccccc1-c1cnc2[nH]cc(-c3ccn4ncc(-c5cccnc5)c4c3)c2c1] | 965 |
| 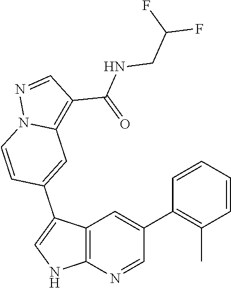 [SMILES: Cc1ccccc1-c1cnc2[nH]cc(-c3ccn4ncc(C(=O)NCC(F)F)c4c3)c2c1] | 966 |
| 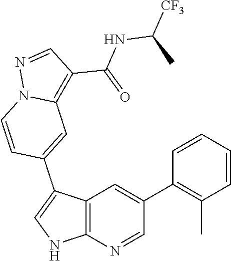 [SMILES: Cc1ccccc1-c1cnc2[nH]cc(-c3ccn4ncc(C(=O)N[C@H](C)C(F)(F)F)c4c3)c2c1] | 967 |
| 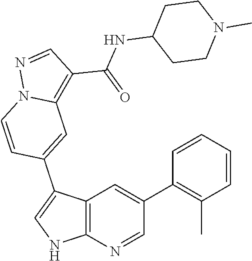 [SMILES: Cc1ccccc1-c1cnc2[nH]cc(-c3ccn4ncc(C(=O)NC5CCN(C)CC5)c4c3)c2c1] | 968 |
| 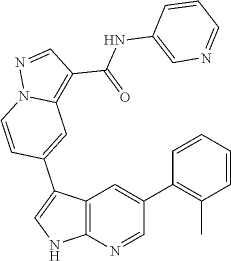 [SMILES: Cc1ccccc1-c1cnc2[nH]cc(-c3ccn4ncc(C(=O)Nc5cccnc5)c4c3)c2c1] | 969 |
| 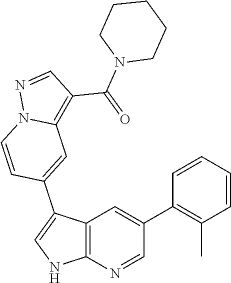 [SMILES: Cc1ccccc1-c1cnc2[nH]cc(-c3ccn4ncc(C(=O)N5CCCCC5)c4c3)c2c1] | 970 |
| 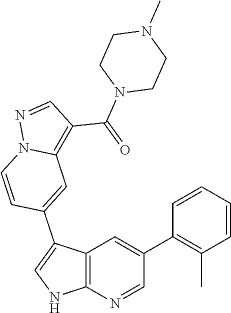 [SMILES: Cc1ccccc1-c1cnc2[nH]cc(-c3ccn4ncc(C(=O)N5CCN(C)CC5)c4c3)c2c1] | 971 |
| 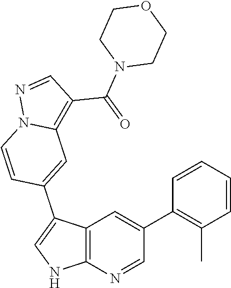 [SMILES: Cc1ccccc1-c1cnc2[nH]cc(-c3ccn4ncc(C(=O)N5CCOCC5)c4c3)c2c1] | 972 |
| 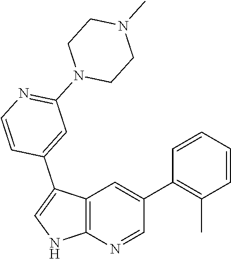 [SMILES: Cc1ccccc1-c1cnc2[nH]cc(-c3ccnc(N4CCN(C)CC4)c3)c2c1] | 973 |
| 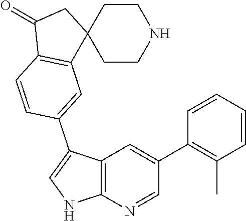 [SMILES: Cc1ccccc1-c1cnc2[nH]cc(-c3ccc4c(c3)C3(CCNCC3)CC4=O)c2c1] | 974 |
| 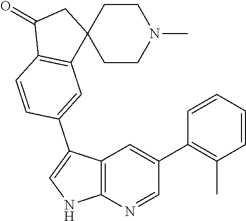 [SMILES: Cc1ccccc1-c1cnc2[nH]cc(-c3ccc4c(c3)C3(CCN(C)CC3)CC4=O)c2c1] | 975 |
| 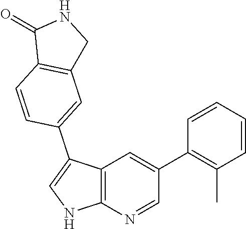 [SMILES: Cc1ccccc1-c1cnc2[nH]cc(-c3ccc4c(c3)CNC4=O)c2c1] | 976 |
| 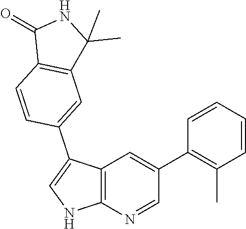 [SMILES: Cc1ccccc1-c1cnc2[nH]cc(-c3ccc4c(c3)C(C)(C)NC4=O)c2c1] | 977 |
| 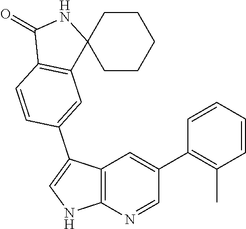 [SMILES: Cc1ccccc1-c1cnc2[nH]cc(-c3ccc4c(c3)C3(CCCCC3)NC4=O)c2c1] | 978 |
| 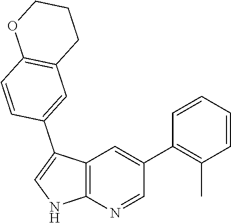 [SMILES: Cc1ccccc1-c1cnc2[nH]cc(-c3ccc4c(c3)CCCO4)c2c1] | 979 |
| 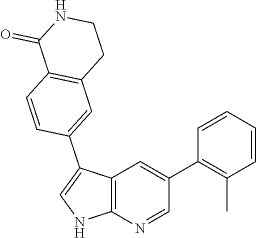 [SMILES: Cc1ccccc1-c1cnc2[nH]cc(-c3ccc4c(c3)CCNC4=O)c2c1] | 980 |
| 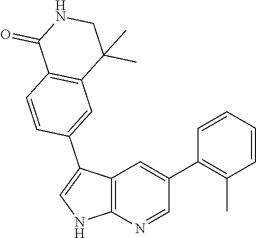 [SMILES: Cc1ccccc1-c1cnc2[nH]cc(-c3ccc4c(c3)C(C)(C)CNC4=O)c2c1] | 981 |
| 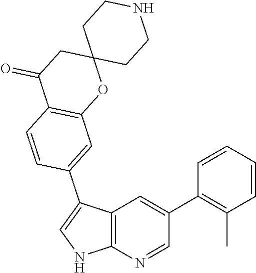 [SMILES: Cc1ccccc1-c1cnc2[nH]cc(-c3ccc4c(c3)OC3(CCNCC3)CC4=O)c2c1] | 982 |
| 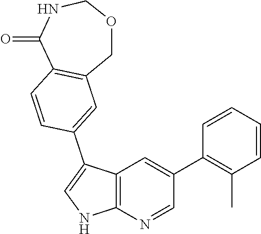 [SMILES: Cc1ccccc1-c1cnc2[nH]cc(-c3ccc4c(c3)COCNC4=O)c2c1] | 983 |
| 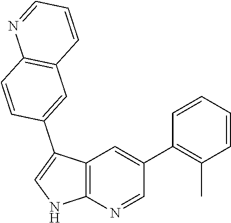 [SMILES: Cc1ccccc1-c1cnc2[nH]cc(-c3ccc4ncccc4c3)c2c1] | 984 |
| 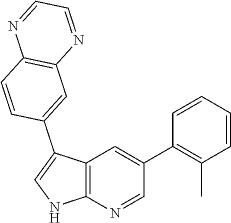 [SMILES: Cc1ccccc1-c1cnc2[nH]cc(-c3ccc4nccnc4c3)c2c1] | 985 |
| 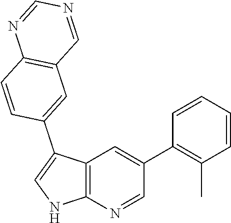 [SMILES: Cc1ccccc1-c1cnc2[nH]cc(-c3ccc4ncncc4c3)c2c1] | 986 |
| 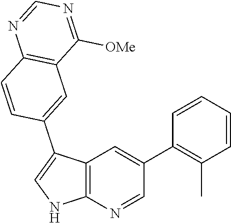 [SMILES: COc1ncnc2ccc(-c3c[nH]c4ncc(-c5ccccc5C)cc34)cc12] | 987 |
| 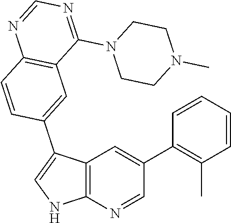 [SMILES: Cc1ccccc1-c1cnc2[nH]cc(-c3ccc4ncnc(N5CCN(C)CC5)c4c3)c2c1] | 988 |
| 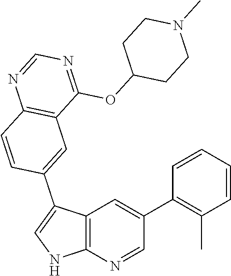 [SMILES: Cc1ccccc1-c1cnc2[nH]cc(-c3ccc4ncnc(OC5CCN(C)CC5)c4c3)c2c1] | 989 |
| 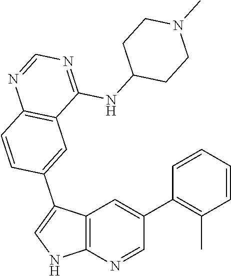 [SMILES: Cc1ccccc1-c1cnc2[nH]cc(-c3ccc4ncnc(NC5CCN(C)CC5)c4c3)c2c1] | 990 |
| 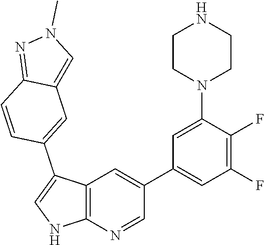 [SMILES: Cn1cc2cc(-c3c[nH]c4ncc(-c5cc(F)c(F)c(N6CCNCC6)c5)cc34)ccc2n1] | 991 |
| 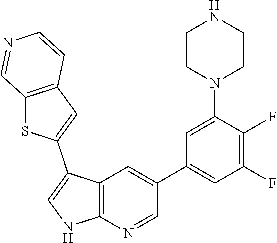 [SMILES: Fc1cc(-c2cnc3[nH]cc(-c4cc5ccncc5s4)c3c2)cc(N2CCNCC2)c1F] | 992 |
| 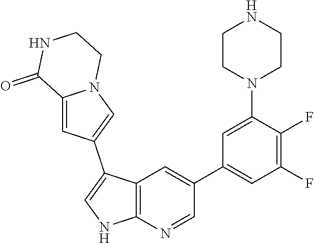 [SMILES: O=C1NCCn2cc(-c3c[nH]c4ncc(-c5cc(F)c(F)c(N6CCNCC6)c5)cc34)cc21] | 993 |
| 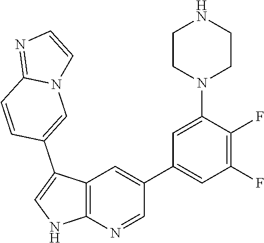 [SMILES: Fc1cc(-c2cnc3[nH]cc(-c4ccc5nccn5c4)c3c2)cc(N2CCNCC2)c1F] | 994 |
| 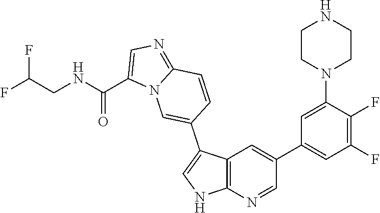 [SMILES: O=C(NCC(F)F)c1cnc2ccc(-c3c[nH]c4ncc(-c5cc(F)c(F)c(N6CCNCC6)c5)cc34)cn12] | 995 |
| 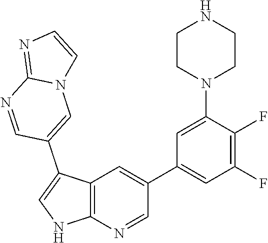 [SMILES: Fc1cc(-c2cnc3[nH]cc(-c4cnc5nccn5c4)c3c2)cc(N2CCNCC2)c1F] | 996 |
| 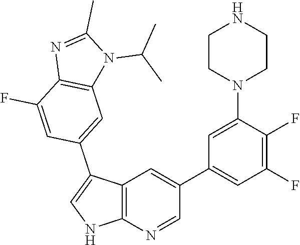 [SMILES: Cc1nc2c(F)cc(-c3c[nH]c4ncc(-c5cc(F)c(F)c(N6CCNCC6)c5)cc34)cc2n1C(C)C] | 997 |
| 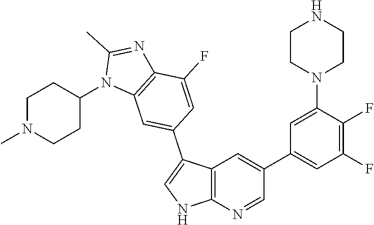 [SMILES: Cc1nc2c(F)cc(-c3c[nH]c4ncc(-c5cc(F)c(F)c(N6CCNCC6)c5)cc34)cc2n1C1CCN(C)CC1] | 998 |
| 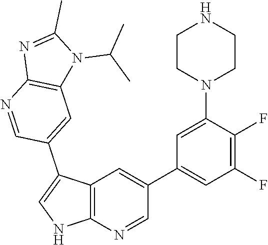 [SMILES: Cc1nc2ncc(-c3c[nH]c4ncc(-c5cc(F)c(F)c(N6CCNCC6)c5)cc34)cc2n1C(C)C] | 999 |
| 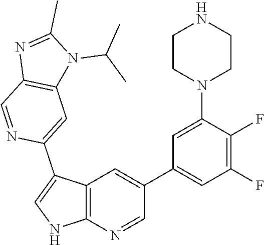 [SMILES: Cc1nc2cnc(-c3c[nH]c4ncc(-c5cc(F)c(F)c(N6CCNCC6)c5)cc34)cc2n1C(C)C] | 1000 |
| 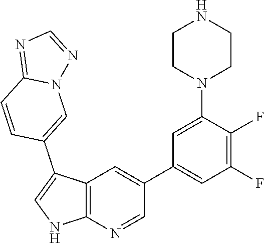 [SMILES: Fc1cc(-c2cnc3[nH]cc(-c4ccc5ncnn5c4)c3c2)cc(N2CCNCC2)c1F] | 1001 |
| 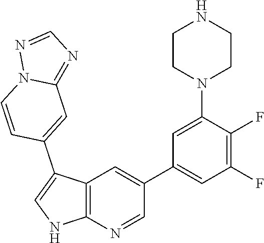 [SMILES: Fc1cc(-c2cnc3[nH]cc(-c4ccn5ncnc5c4)c3c2)cc(N2CCNCC2)c1F] | 1002 |
| 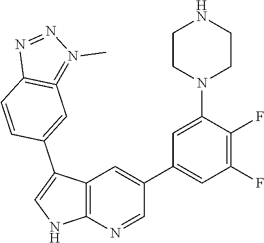 | 1003 |
| 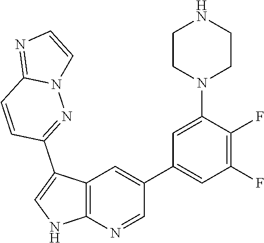 [SMILES: Fc1cc(-c2cnc3[nH]cc(-c4ccc5nccn5n4)c3c2)cc(N2CCNCC2)c1F] | 1004 |
| 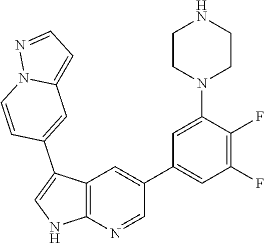 [SMILES: Fc1cc(-c2cnc3[nH]cc(-c4ccn5nccc5c4)c3c2)cc(N2CCNCC2)c1F] | 1005 |
| 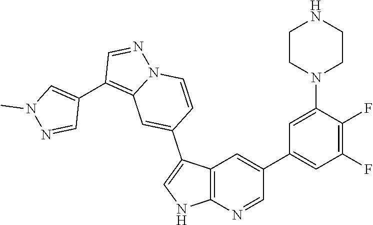 [SMILES: Cn1cc(-c2cnn3ccc(-c4c[nH]c5ncc(-c6cc(F)c(F)c(N7CCNCC7)c6)cc45)cc23)cn1] | 1006 |
| 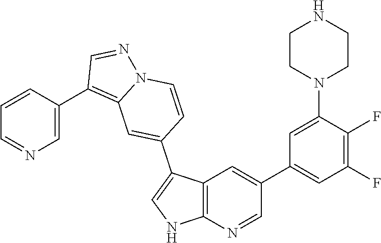 [SMILES: Fc1cc(-c2cnc3[nH]cc(-c4ccn5ncc(-c6cccnc6)c5c4)c3c2)cc(N2CCNCC2)c1F] | 1007 |
| 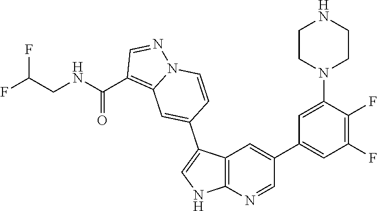 [SMILES: O=C(NCC(F)F)c1cnn2ccc(-c3c[nH]c4ncc(-c5cc(F)c(F)c(N6CCNCC6)c5)cc34)cc12] | 1008 |
| 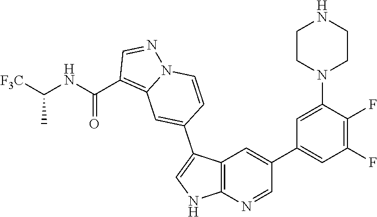 [SMILES: C[C@@H](NC(=O)c1cnn2ccc(-c3c[nH]c4ncc(-c5cc(F)c(F)c(N6CCNCC6)c5)cc34)cc12)C(F)(F)F] | 1009 |
| 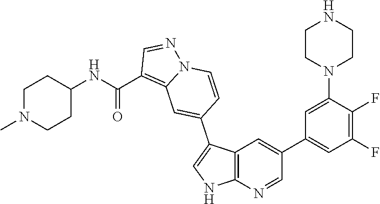 [SMILES: CN1CCC(NC(=O)c2cnn3ccc(-c4c[nH]c5ncc(-c6cc(F)c(F)c(N7CCNCC7)c6)cc45)cc23)CC1] | 1010 |
| 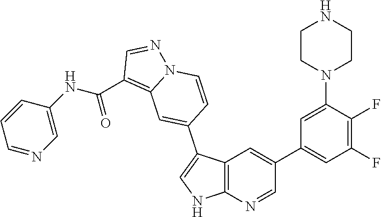 [SMILES: O=C(Nc1cccnc1)c1cnn2ccc(-c3c[nH]c4ncc(-c5cc(F)c(F)c(N6CCNCC6)c5)cc34)cc12] | 1011 |
| 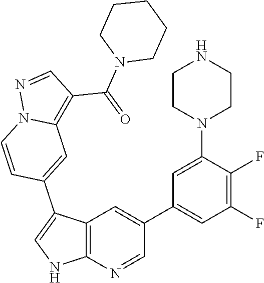 [SMILES: O=C(c1cnn2ccc(-c3c[nH]c4ncc(-c5cc(F)c(F)c(N6CCNCC6)c5)cc34)cc12)N1CCCCC1] | 1012 |
| 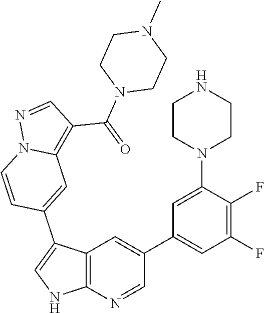 [SMILES: CN1CCN(C(=O)c2cnn3ccc(-c4c[nH]c5ncc(-c6cc(F)c(F)c(N7CCNCC7)c6)cc45)cc23)CC1] | 1013 |
| 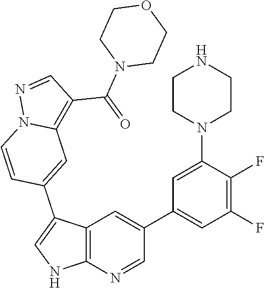 [SMILES: O=C(c1cnn2ccc(-c3c[nH]c4ncc(-c5cc(F)c(F)c(N6CCNCC6)c5)cc34)cc12)N1CCOCC1] | 1014 |
| 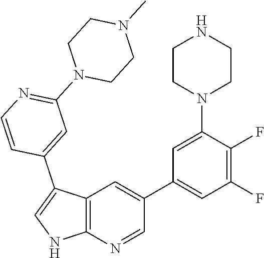 [SMILES: CN1CCN(c2cc(-c3c[nH]c4ncc(-c5cc(F)c(F)c(N6CCNCC6)c5)cc34)ccn2)CC1] | 1015 |
| 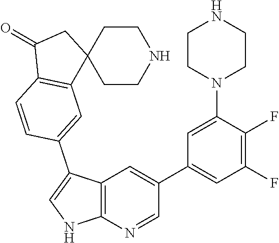 [SMILES: O=C1CC2(CCNCC2)c2cc(-c3c[nH]c4ncc(-c5cc(F)c(F)c(N6CCNCC6)c5)cc34)ccc21] | 1016 |
| 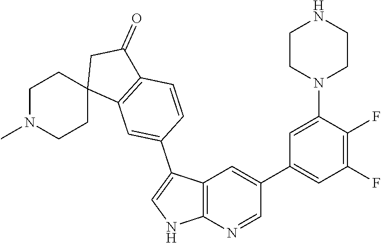 [SMILES: CN1CCC2(CC1)CC(=O)c1ccc(-c3c[nH]c4ncc(-c5cc(F)c(F)c(N6CCNCC6)c5)cc34)cc12] | 1017 |
| 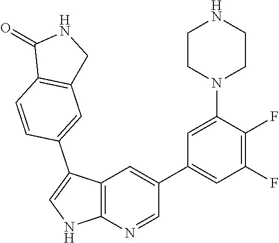 [SMILES: O=C1NCc2cc(-c3c[nH]c4ncc(-c5cc(F)c(F)c(N6CCNCC6)c5)cc34)ccc21] | 1018 |
| 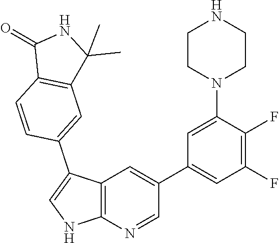 [SMILES: CC1(C)NC(=O)c2ccc(-c3c[nH]c4ncc(-c5cc(F)c(F)c(N6CCNCC6)c5)cc34)cc21] | 1019 |
| 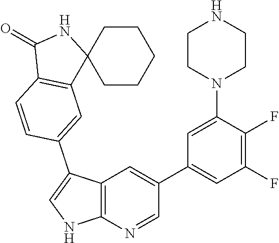 [SMILES: O=C1NC2(CCCCC2)c2cc(-c3c[nH]c4ncc(-c5cc(F)c(F)c(N6CCNCC6)c5)cc34)ccc21] | 1020 |
| 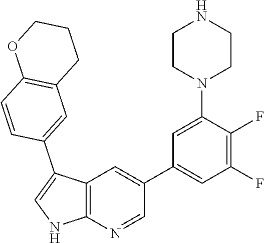 [SMILES: Fc1cc(-c2cnc3[nH]cc(-c4ccc5c(c4)CCCO5)c3c2)cc(N2CCNCC2)c1F] | 1021 |
| 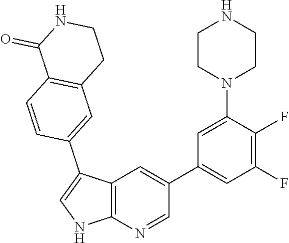 [SMILES: O=C1NCCc2cc(-c3c[nH]c4ncc(-c5cc(F)c(F)c(N6CCNCC6)c5)cc34)ccc21] | 1022 |
| 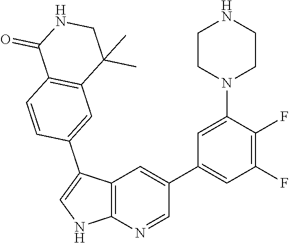 [SMILES: CC1(C)CNC(=O)c2ccc(-c3c[nH]c4ncc(-c5cc(F)c(F)c(N6CCNCC6)c5)cc34)cc21] | 1023 |
| 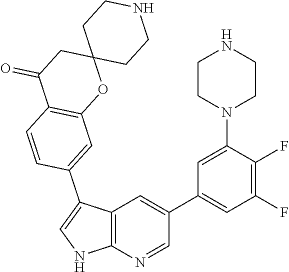 [SMILES: O=C1CC2(CCNCC2)Oc2cc(-c3c[nH]c4ncc(-c5cc(F)c(F)c(N6CCNCC6)c5)cc34)ccc21] | 1024 |
| 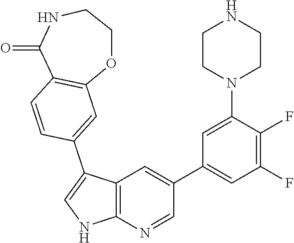 [SMILES: O=C1NCCOc2cc(-c3c[nH]c4ncc(-c5cc(F)c(F)c(N6CCNCC6)c5)cc34)ccc21] | 1025 |
| 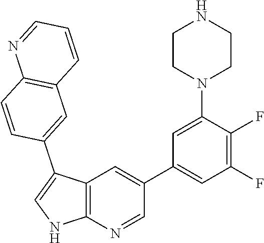 [SMILES: Fc1cc(-c2cnc3[nH]cc(-c4ccc5ncccc5c4)c3c2)cc(N2CCNCC2)c1F] | 1026 |
| 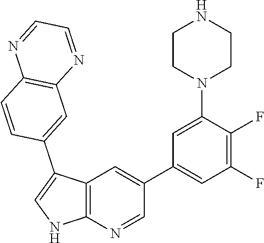 [SMILES: Fc1cc(-c2cnc3[nH]cc(-c4ccc5nccnc5c4)c3c2)cc(N2CCNCC2)c1F] | 1027 |
| 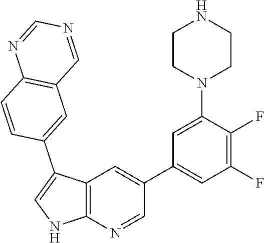 [SMILES: Fc1cc(-c2cnc3[nH]cc(-c4ccc5ncncc5c4)c3c2)cc(N2CCNCC2)c1F] | 1028 |
| 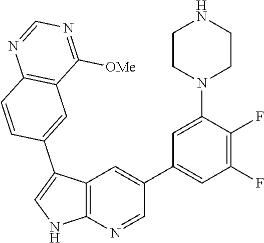 [SMILES: COc1ncnc2ccc(-c3c[nH]c4ncc(-c5cc(F)c(F)c(N6CCNCC6)c5)cc34)cc12] | 1029 |
| 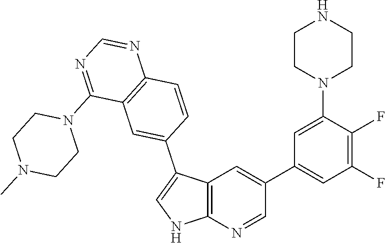 [SMILES: CN1CCN(c2ncnc3ccc(-c4c[nH]c5ncc(-c6cc(F)c(F)c(N7CCNCC7)c6)cc45)cc23)CC1] | 1030 |
| 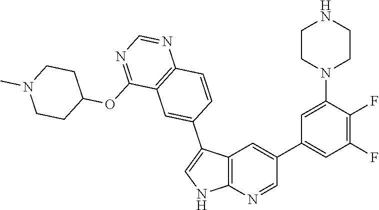 [SMILES: CN1CCC(Oc2ncnc3ccc(-c4c[nH]c5ncc(-c6cc(F)c(F)c(N7CCNCC7)c6)cc45)cc23)CC1] | 1031 |
| 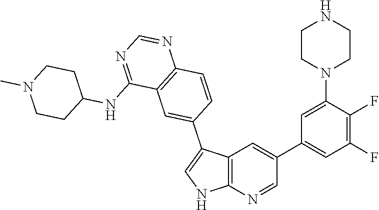 [SMILES: CN1CCC(Nc2ncnc3ccc(-c4c[nH]c5ncc(-c6cc(F)c(F)c(N7CCNCC7)c6)cc45)cc23)CC1] | 1032 |
| 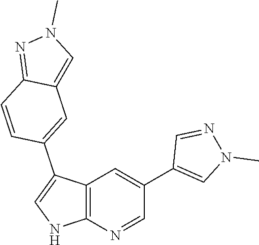 [SMILES: Cn1cc(-c2cnc3[nH]cc(-c4ccc5nn(C)cc5c4)c3c2)cn1] | 1033 |
| 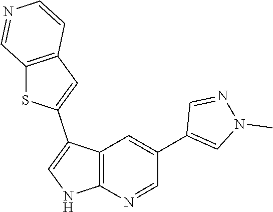 [SMILES: Cn1cc(-c2cnc3[nH]cc(-c4cc5ccncc5s4)c3c2)cn1] | 1034 |
| 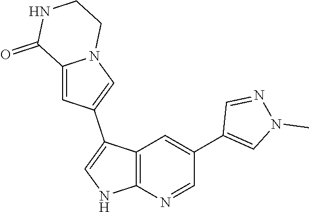 [SMILES: Cn1cc(-c2cnc3[nH]cc(-c4cc5n(c4)CCNC5=O)c3c2)cn1] | 1035 |
| 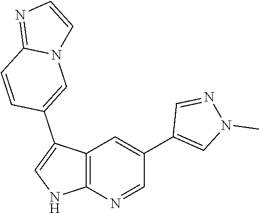 [SMILES: Cn1cc(-c2cnc3[nH]cc(-c4ccc5nccn5c4)c3c2)cn1] | 1036 |
| 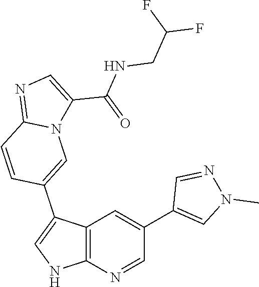 [SMILES: Cn1cc(-c2cnc3[nH]cc(-c4ccc5ncc(C(=O)NCC(F)F)n5c4)c3c2)cn1] | 1037 |
| 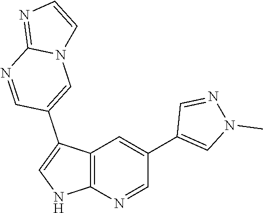 [SMILES: Cn1cc(-c2cnc3[nH]cc(-c4cnc5nccn5c4)c3c2)cn1] | 1038 |
| 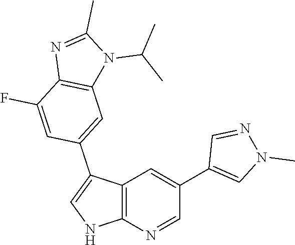 [SMILES: Cc1nc2c(F)cc(-c3c[nH]c4ncc(-c5cnn(C)c5)cc34)cc2n1C(C)C] | 1039 |
| 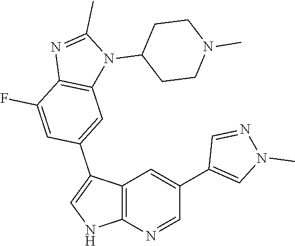 [SMILES: Cc1nc2c(F)cc(-c3c[nH]c4ncc(-c5cnn(C)c5)cc34)cc2n1C1CCN(C)CC1] | 1040 |
| 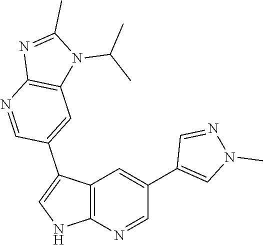 [SMILES: Cc1nc2ncc(-c3c[nH]c4ncc(-c5cnn(C)c5)cc34)cc2n1C(C)C] | 1041 |
| 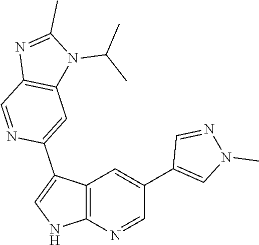 [SMILES: Cc1nc2cnc(-c3c[nH]c4ncc(-c5cnn(C)c5)cc34)cc2n1C(C)C] | 1042 |
| 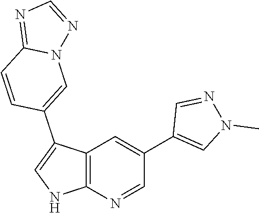 [SMILES: Cn1cc(-c2cnc3[nH]cc(-c4ccc5ncnn5c4)c3c2)cn1] | 1043 |
| 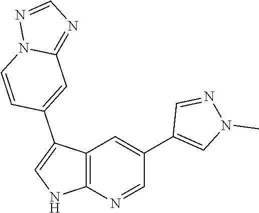 [SMILES: Cn1cc(-c2cnc3[nH]cc(-c4ccn5ncnc5c4)c3c2)cn1] | 1044 |
| 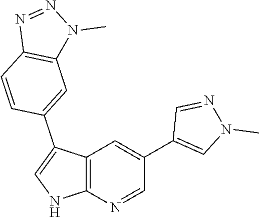 [SMILES: Cn1cc(-c2cnc3[nH]cc(-c4ccc5nnn(C)c5c4)c3c2)cn1] | 1045 |
| 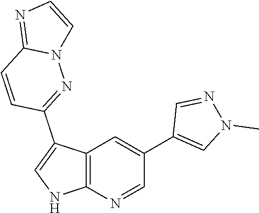 [SMILES: Cn1cc(-c2cnc3[nH]cc(-c4ccc5nccn5n4)c3c2)cn1] | 1046 |
| 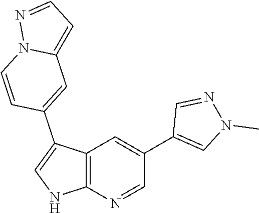 [SMILES: Cn1cc(-c2cnc3[nH]cc(-c4ccn5nccc5c4)c3c2)cn1] | 1047 |
| 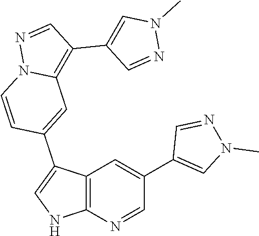 [SMILES: Cn1cc(-c2cnc3[nH]cc(-c4ccn5ncc(-c6cnn(C)c6)c5c4)c3c2)cn1] | 1048 |
| 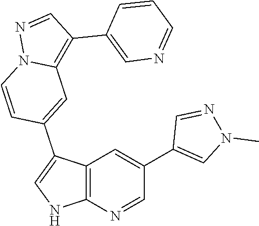 [SMILES: Cn1cc(-c2cnc3[nH]cc(-c4ccn5ncc(-c6cccnc6)c5c4)c3c2)cn1] | 1049 |
| 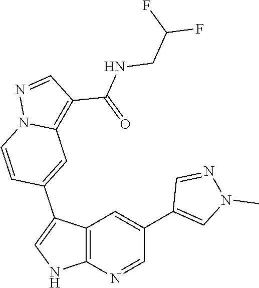 [SMILES: Cn1cc(-c2cnc3[nH]cc(-c4ccn5ncc(C(=O)NCC(F)F)c5c4)c3c2)cn1] | 1050 |
| 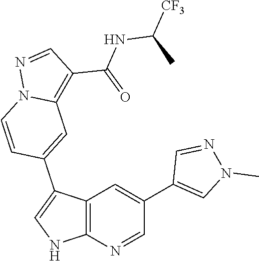 [SMILES: C[C@@H](NC(=O)c1cnn2ccc(-c3c[nH]c4ncc(-c5cnn(C)c5)cc34)cc12)C(F)(F)F] | 1051 |
| 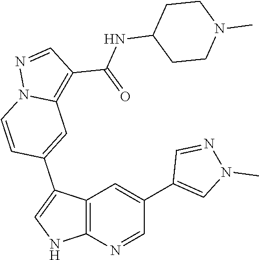 [SMILES: CN1CCC(NC(=O)c2cnn3ccc(-c4c[nH]c5ncc(-c6cnn(C)c6)cc45)cc23)CC1] | 1052 |
| 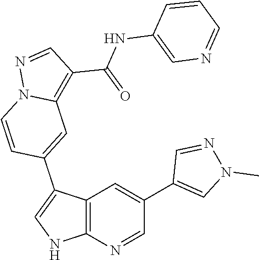 [SMILES: Cn1cc(-c2cnc3[nH]cc(-c4ccn5ncc(C(=O)Nc6cccnc6)c5c4)c3c2)cn1] | 1053 |
| 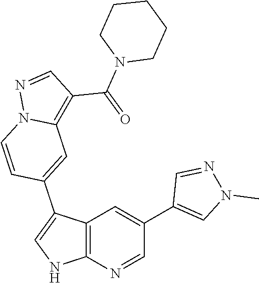 [SMILES: Cn1cc(-c2cnc3[nH]cc(-c4ccn5ncc(C(=O)N6CCCCC6)c5c4)c3c2)cn1] | 1054 |
| 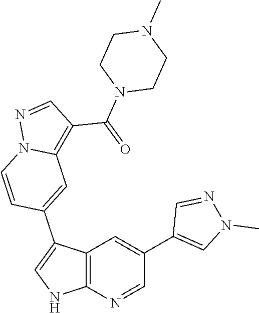 [SMILES: CN1CCN(C(=O)c2cnn3ccc(-c4c[nH]c5ncc(-c6cnn(C)c6)cc45)cc23)CC1] | 1055 |
| 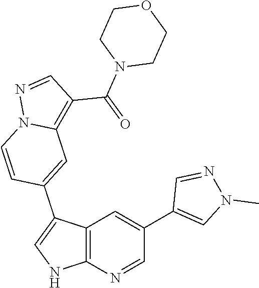 [SMILES: Cn1cc(-c2cnc3[nH]cc(-c4ccn5ncc(C(=O)N6CCOCC6)c5c4)c3c2)cn1] | 1056 |
| 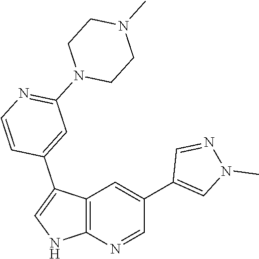 [SMILES: CN1CCN(c2cc(-c3c[nH]c4ncc(-c5cnn(C)c5)cc34)ccn2)CC1] | 1057 |
| 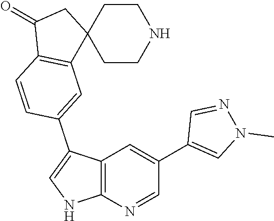 [SMILES: Cn1cc(-c2cnc3[nH]cc(-c4ccc5c(c4)C4(CCNCC4)CC5=O)c3c2)cn1] | 1058 |
| 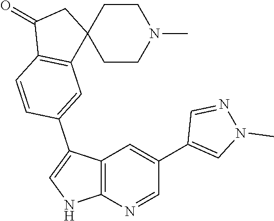 [SMILES: CN1CCC2(CC1)CC(=O)c1ccc(-c3c[nH]c4ncc(-c5cnn(C)c5)cc34)cc12] | 1059 |
| 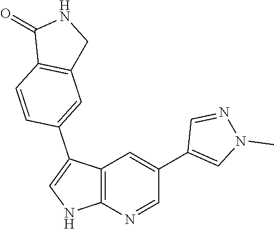 [SMILES: Cn1cc(-c2cnc3[nH]cc(-c4ccc5c(c4)CNC5=O)c3c2)cn1] | 1060 |
| 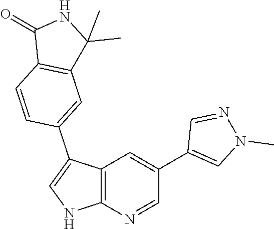 [SMILES: Cn1cc(-c2cnc3[nH]cc(-c4ccc5c(c4)C(C)(C)NC5=O)c3c2)cn1] | 1061 |
| 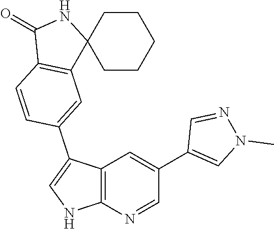 [SMILES: Cn1cc(-c2cnc3[nH]cc(-c4ccc5c(c4)C4(CCCCC4)NC5=O)c3c2)cn1] | 1062 |
| 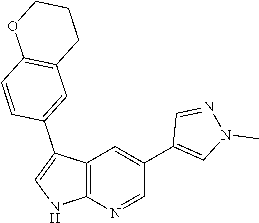 [SMILES: Cn1cc(-c2cnc3[nH]cc(-c4ccc5c(c4)CCCO5)c3c2)cn1] | 1063 |
| 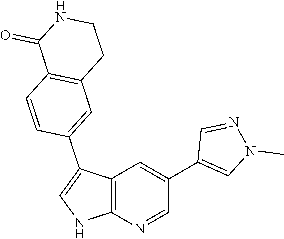 [SMILES: Cn1cc(-c2cnc3[nH]cc(-c4ccc5c(c4)CCNC5=O)c3c2)cn1] | 1064 |
| 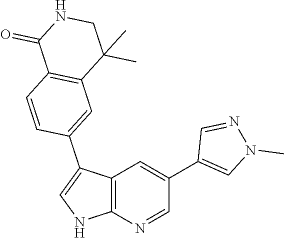 [SMILES: Cn1cc(-c2cnc3[nH]cc(-c4ccc5c(c4)C(C)(C)CNC5=O)c3c2)cn1] | 1065 |
| 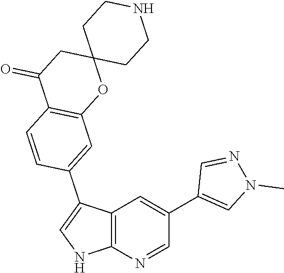 [SMILES: Cn1cc(-c2cnc3[nH]cc(-c4ccc5c(c4)OC4(CCNCC4)CC5=O)c3c2)cn1] | 1066 |
| 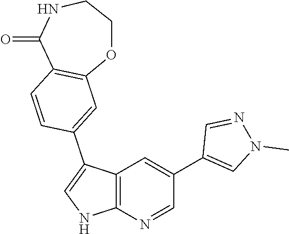 [SMILES: Cn1cc(-c2cnc3[nH]cc(-c4ccc5c(c4)OCCNC5=O)c3c2)cn1] | 1067 |
| 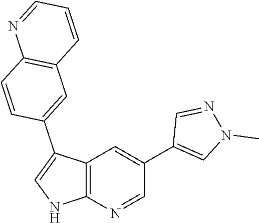 [SMILES: Cn1cc(-c2cnc3[nH]cc(-c4ccc5ncccc5c4)c3c2)cn1] | 1068 |
| 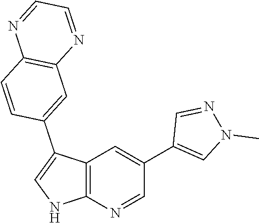 [SMILES: Cn1cc(-c2cnc3[nH]cc(-c4ccc5nccnc5c4)c3c2)cn1] | 1069 |
| 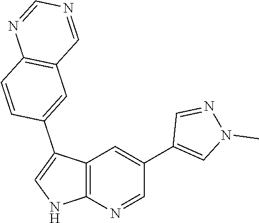 [SMILES: Cn1cc(-c2cnc3[nH]cc(-c4ccc5ncncc5c4)c3c2)cn1] | 1070 |
| 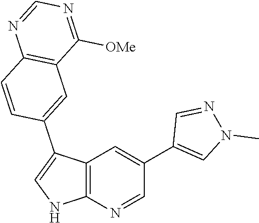 [SMILES: COc1ncnc2ccc(-c3c[nH]c4ncc(-c5cnn(C)c5)cc34)cc12] | 1071 |
| 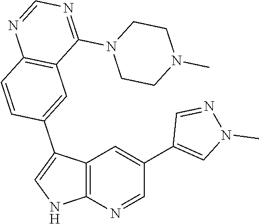 [SMILES: CN1CCN(c2ncnc3ccc(-c4c[nH]c5ncc(-c6cnn(C)c6)cc45)cc23)CC1] | 1072 |
| 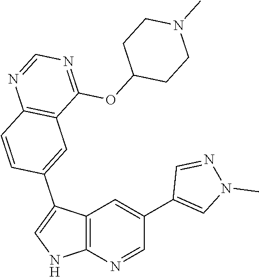 [SMILES: CN1CCC(Oc2ncnc3ccc(-c4c[nH]c5ncc(-c6cnn(C)c6)cc45)cc23)CC1] | 1073 |
| 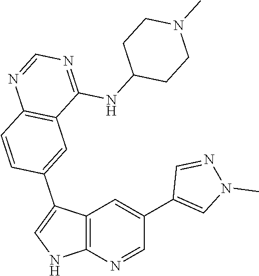 [SMILES: CN1CCC(Nc2ncnc3ccc(-c4c[nH]c5ncc(-c6cnn(C)c6)cc45)cc23)CC1] | 1074 |
| 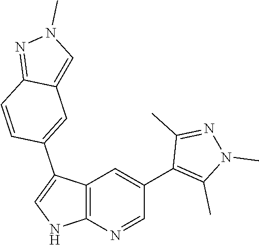 [SMILES: Cc1nn(C)c(C)c1-c1cnc2[nH]cc(-c3ccc4nn(C)cc4c3)c2c1] | 1075 |
| 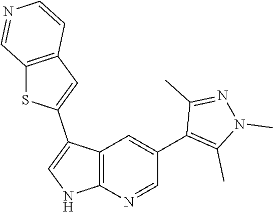 [SMILES: Cc1nn(C)c(C)c1-c1cnc2[nH]cc(-c3cc4ccncc4s3)c2c1] | 1076 |
| 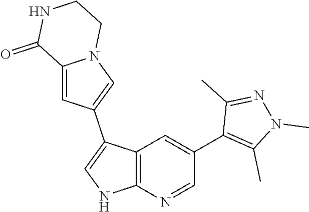 [SMILES: Cc1nn(C)c(C)c1-c1cnc2[nH]cc(-c3cc4n(c3)CCNC4=O)c2c1] | 1077 |
| 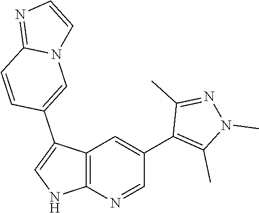 [SMILES: Cc1nn(C)c(C)c1-c1cnc2[nH]cc(-c3ccc4nccn4c3)c2c1] | 1078 |
| 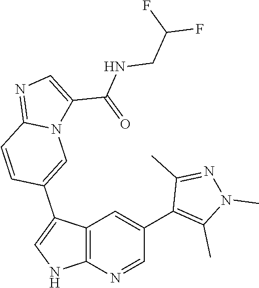 [SMILES: Cc1nn(C)c(C)c1-c1cnc2[nH]cc(-c3ccc4ncc(C(=O)NCC(F)F)n4c3)c2c1] | 1079 |
| 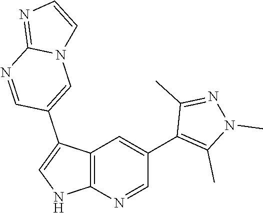 [SMILES: Cc1nn(C)c(C)c1-c1cnc2[nH]cc(-c3cnc4nccn4c3)c2c1] | 1080 |
| 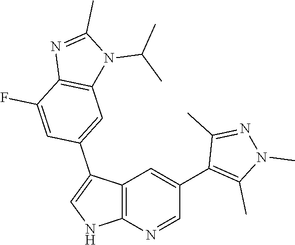 [SMILES: Cc1nn(C)c(C)c1-c1cnc2[nH]cc(-c3cc(F)c4nc(C)n(C(C)C)c4c3)c2c1] | 1081 |
| 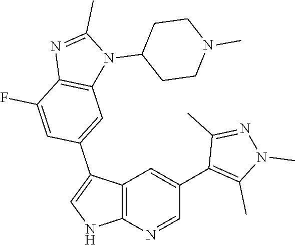 [SMILES: Cc1nn(C)c(C)c1-c1cnc2[nH]cc(-c3cc(F)c4nc(C)n(C5CCN(C)CC5)c4c3)c2c1] | 1082 |
| 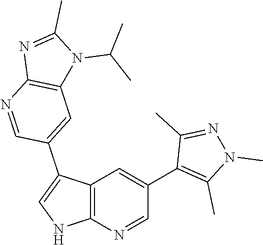 [SMILES: Cc1nn(C)c(C)c1-c1cnc2[nH]cc(-c3cnc4nc(C)n(C(C)C)c4c3)c2c1] | 1083 |
| 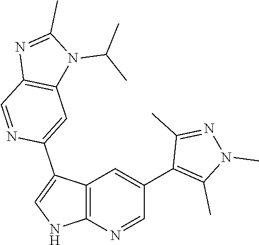 [SMILES: Cc1nn(C)c(C)c1-c1cnc2[nH]cc(-c3cc4c(cn3)nc(C)n4C(C)C)c2c1] | 1084 |
| 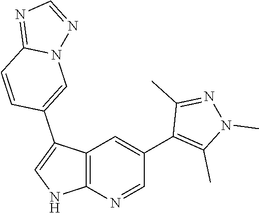 [SMILES: Cc1nn(C)c(C)c1-c1cnc2[nH]cc(-c3ccc4ncnn4c3)c2c1] | 1085 |
| 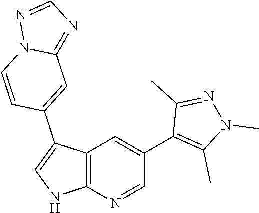 [SMILES: Cc1nn(C)c(C)c1-c1cnc2[nH]cc(-c3ccn4ncnc4c3)c2c1] | 1086 |
| 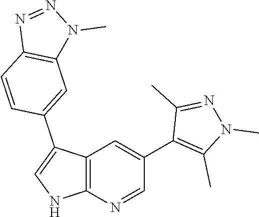 [SMILES: Cc1nn(C)c(C)c1-c1cnc2[nH]cc(-c3ccc4nnn(C)c4c3)c2c1] | 1087 |
| 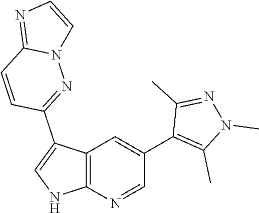 [SMILES: Cc1nn(C)c(C)c1-c1cnc2[nH]cc(-c3ccc4nccn4n3)c2c1] | 1088 |
| 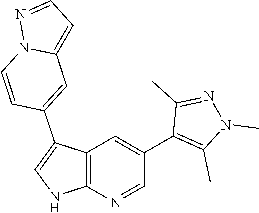 [SMILES: Cc1nn(C)c(C)c1-c1cnc2[nH]cc(-c3ccn4nccc4c3)c2c1] | 1089 |
| 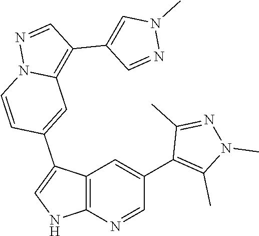 [SMILES: Cc1nn(C)c(C)c1-c1cnc2[nH]cc(-c3ccn4ncc(-c5cnn(C)c5)c4c3)c2c1] | 1090 |
| 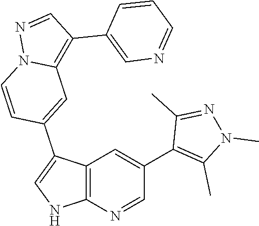 [SMILES: Cc1nn(C)c(C)c1-c1cnc2[nH]cc(-c3ccn4ncc(-c5cccnc5)c4c3)c2c1] | 1091 |
| 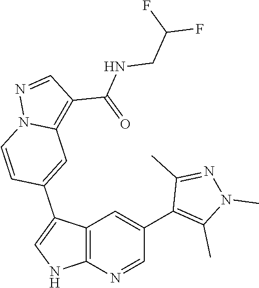 [SMILES: Cc1nn(C)c(C)c1-c1cnc2[nH]cc(-c3ccn4ncc(C(=O)NCC(F)F)c4c3)c2c1] | 1092 |
| 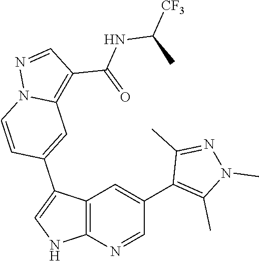 [SMILES: Cc1nn(C)c(C)c1-c1cnc2[nH]cc(-c3ccn4ncc(C(=O)N[C@H](C)C(F)(F)F)c4c3)c2c1] | 1093 |
| 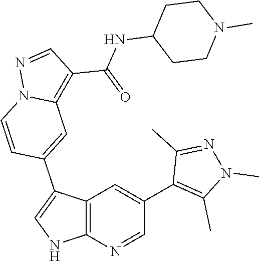 [SMILES: Cc1nn(C)c(C)c1-c1cnc2[nH]cc(-c3ccn4ncc(C(=O)NC5CCN(C)CC5)c4c3)c2c1] | 1094 |
| 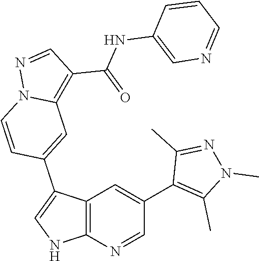 [SMILES: Cc1nn(C)c(C)c1-c1cnc2[nH]cc(-c3ccn4ncc(C(=O)Nc5cccnc5)c4c3)c2c1] | 1095 |
| 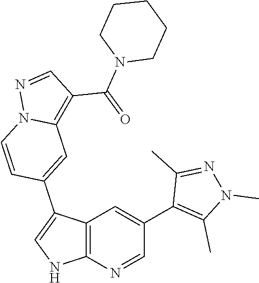 [SMILES: Cc1nn(C)c(C)c1-c1cnc2[nH]cc(-c3ccn4ncc(C(=O)N5CCCCC5)c4c3)c2c1] | 1096 |
| 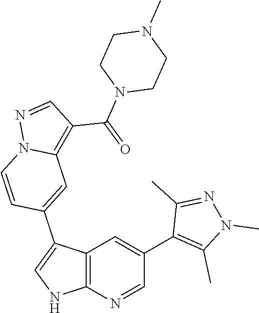 [SMILES: Cc1nn(C)c(C)c1-c1cnc2[nH]cc(-c3ccn4ncc(C(=O)N5CCN(C)CC5)c4c3)c2c1] | 1097 |
| 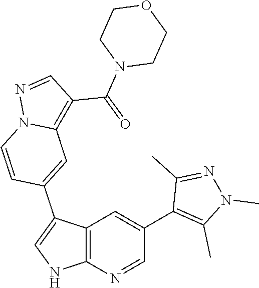 [SMILES: Cc1nn(C)c(C)c1-c1cnc2[nH]cc(-c3ccn4ncc(C(=O)N5CCOCC5)c4c3)c2c1] | 1098 |
| 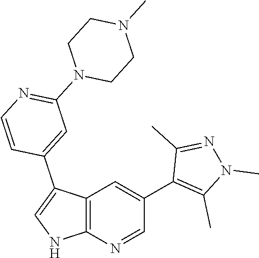 [SMILES: Cc1nn(C)c(C)c1-c1cnc2[nH]cc(-c3ccnc(N4CCN(C)CC4)c3)c2c1] | 1099 |
| 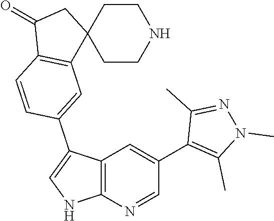 [SMILES: Cc1nn(C)c(C)c1-c1cnc2[nH]cc(-c3ccc4c(c3)C3(CCNCC3)CC4=O)c2c1] | 1100 |
| 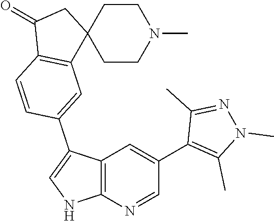 [SMILES: Cc1nn(C)c(C)c1-c1cnc2[nH]cc(-c3ccc4c(c3)C3(CCN(C)CC3)CC4=O)c2c1] | 1101 |
| 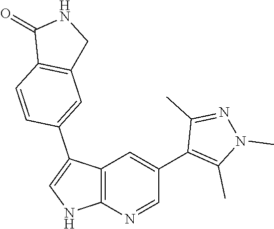 [SMILES: Cc1nn(C)c(C)c1-c1cnc2[nH]cc(-c3ccc4c(c3)CNC4=O)c2c1] | 1102 |
| 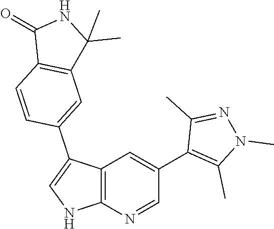 [SMILES: Cc1nn(C)c(C)c1-c1cnc2[nH]cc(-c3ccc4c(c3)C(C)(C)NC4=O)c2c1] | 1103 |
| 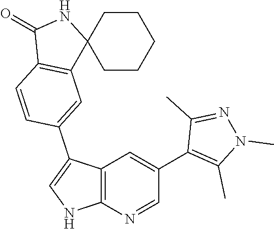 [SMILES: Cc1nn(C)c(C)c1-c1cnc2[nH]cc(-c3ccc4c(c3)C3(CCCCC3)NC4=O)c2c1] | 1104 |
| 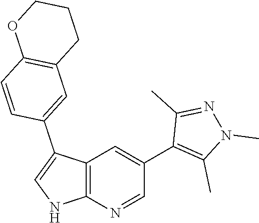 [SMILES: Cc1nn(C)c(C)c1-c1cnc2[nH]cc(-c3ccc4c(c3)CCCO4)c2c1] | 1105 |
| 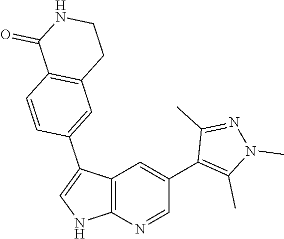 [SMILES: Cc1nn(C)c(C)c1-c1cnc2[nH]cc(-c3ccc4c(c3)CCNC4=O)c2c1] | 1106 |
| 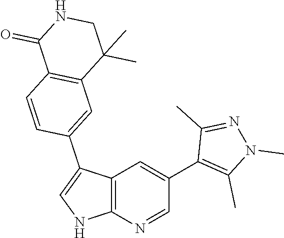 [SMILES: Cc1nn(C)c(C)c1-c1cnc2[nH]cc(-c3ccc4c(c3)C(C)(C)CNC4=O)c2c1] | 1107 |
| 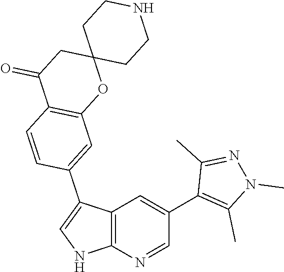 [SMILES: Cc1nn(C)c(C)c1-c1cnc2[nH]cc(-c3ccc4c(c3)OC3(CCNCC3)CC4=O)c2c1] | 1108 |
| 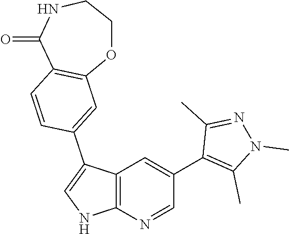 [SMILES: Cc1nn(C)c(C)c1-c1cnc2[nH]cc(-c3ccc4c(c3)OCCNC4=O)c2c1] | 1109 |
| 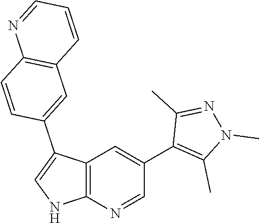 [SMILES: Cc1nn(C)c(C)c1-c1cnc2[nH]cc(-c3ccc4ncccc4c3)c2c1] | 1110 |
| 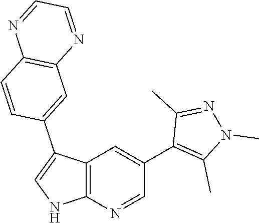 [SMILES: Cc1nn(C)c(C)c1-c1cnc2[nH]cc(-c3ccc4nccnc4c3)c2c1] | 1111 |
| 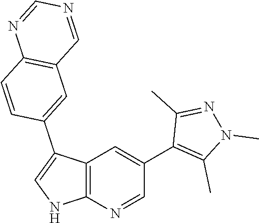 [SMILES: Cc1nn(C)c(C)c1-c1cnc2[nH]cc(-c3ccc4ncncc4c3)c2c1] | 1112 |
| 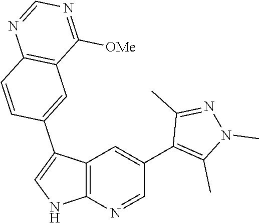 [SMILES: COc1ncnc2ccc(-c3c[nH]c4ncc(-c5c(C)nn(C)c5C)cc34)cc12] | 1113 |
| 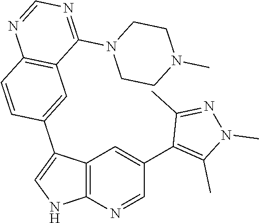 [SMILES: Cc1nn(C)c(C)c1-c1cnc2[nH]cc(-c3ccc4ncnc(N5CCN(C)CC5)c4c3)c2c1] | 1114 |
| 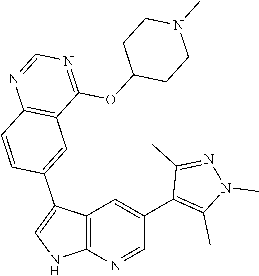 [SMILES: Cc1nn(C)c(C)c1-c1cnc2[nH]cc(-c3ccc4ncnc(OC5CCN(C)CC5)c4c3)c2c1] | 1115 |
| 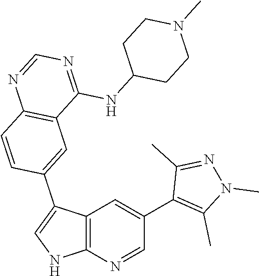 [SMILES: Cc1nn(C)c(C)c1-c1cnc2[nH]cc(-c3ccc4ncnc(NC5CCN(C)CC5)c4c3)c2c1] | 1116 |
| 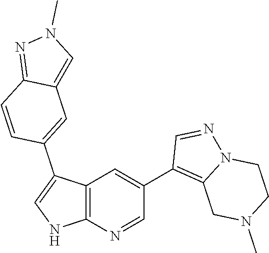 [SMILES: CN1CCn2ncc(-c3cnc4[nH]cc(-c5ccc6nn(C)cc6c5)c4c3)c2C1] | 1117 |
| 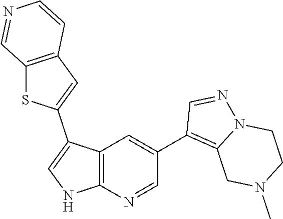 [SMILES: CN1CCn2ncc(-c3cnc4[nH]cc(-c5cc6ccncc6s5)c4c3)c2C1] | 1118 |
| 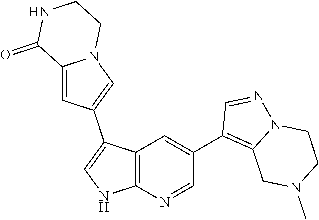 [SMILES: CN1CCn2ncc(-c3cnc4[nH]cc(-c5cc6n(c5)CCNC6=O)c4c3)c2C1] | 1119 |
| 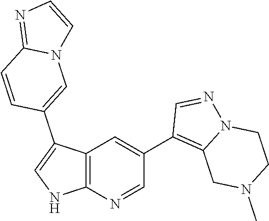 [SMILES: CN1CCn2ncc(-c3cnc4[nH]cc(-c5ccc6nccn6c5)c4c3)c2C1] | 1120 |
| 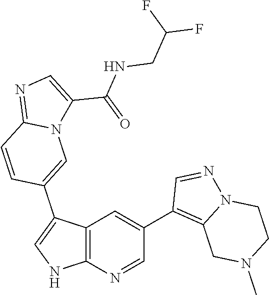 [SMILES: CN1CCn2ncc(-c3cnc4[nH]cc(-c5ccc6ncc(C(=O)NCC(F)F)n6c5)c4c3)c2C1] | 1121 |
| 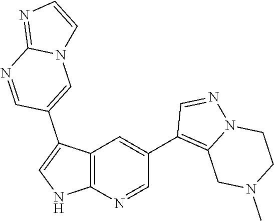 [SMILES: CN1CCn2ncc(-c3cnc4[nH]cc(-c5cnc6nccn6c5)c4c3)c2C1] | 1122 |
| 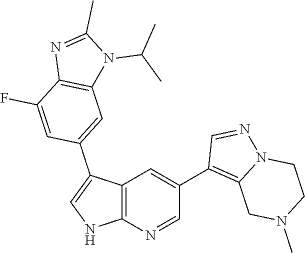 [SMILES: Cc1nc2c(F)cc(-c3c[nH]c4ncc(-c5cnn6c5CN(C)CC6)cc34)cc2n1C(C)C] | 1123 |
| 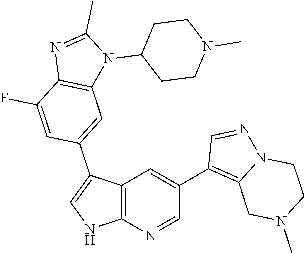 [SMILES: Cc1nc2c(F)cc(-c3c[nH]c4ncc(-c5cnn6c5CN(C)CC6)cc34)cc2n1C1CCN(C)CC1] | 1124 |
| 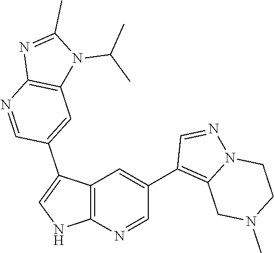 [SMILES: Cc1nc2ncc(-c3c[nH]c4ncc(-c5cnn6c5CN(C)CC6)cc34)cc2n1C(C)C] | 1125 |
| 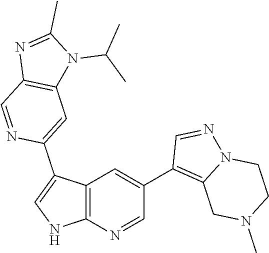 [SMILES: Cc1nc2cnc(-c3c[nH]c4ncc(-c5cnn6c5CN(C)CC6)cc34)cc2n1C(C)C] | 1126 |
| 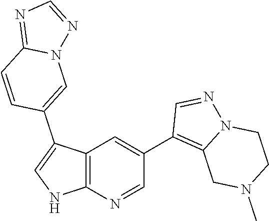 [SMILES: CN1CCn2ncc(-c3cnc4[nH]cc(-c5ccc6ncnn6c5)c4c3)c2C1] | 1127 |
| 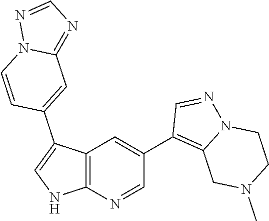 [SMILES: CN1CCn2ncc(-c3cnc4[nH]cc(-c5ccn6ncnc6c5)c4c3)c2C1] | 1128 |
| 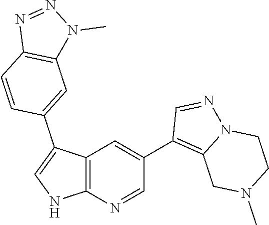 [SMILES: CN1CCn2ncc(-c3cnc4[nH]cc(-c5ccc6nnn(C)c6c5)c4c3)c2C1] | 1129 |
| 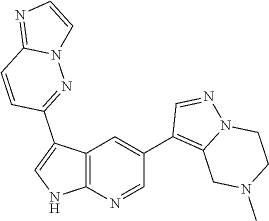 [SMILES: CN1CCn2ncc(-c3cnc4[nH]cc(-c5ccc6nccn6n5)c4c3)c2C1] | 1130 |
| 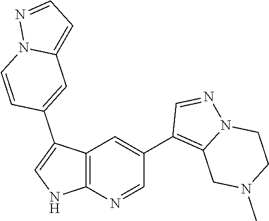 [SMILES: CN1CCn2ncc(-c3cnc4[nH]cc(-c5ccn6nccc6c5)c4c3)c2C1] | 1131 |
| 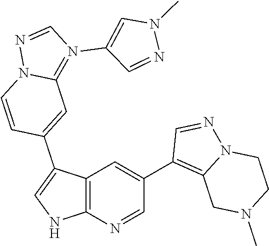 | 1132 |
| 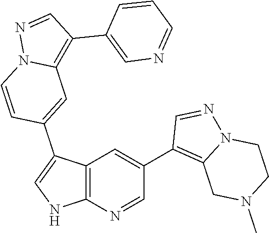 [SMILES: CN1CCn2ncc(-c3cnc4[nH]cc(-c5ccn6ncc(-c7cccnc7)c6c5)c4c3)c2C1] | 1133 |
| 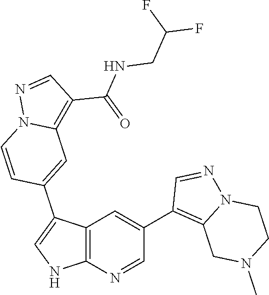 [SMILES: CN1CCn2ncc(-c3cnc4[nH]cc(-c5ccn6ncc(C(=O)NCC(F)F)c6c5)c4c3)c2C1] | 1134 |
| 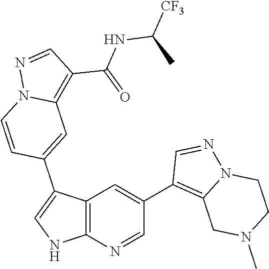 [SMILES: C[C@@H](NC(=O)c1cnn2ccc(-c3c[nH]c4ncc(-c5cnn6c5CN(C)CC6)cc34)cc12)C(F)(F)F] | 1135 |
| 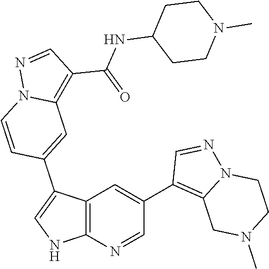 [SMILES: CN1CCC(NC(=O)c2cnn3ccc(-c4c[nH]c5ncc(-c6cnn7c6CN(C)CC7)cc45)cc23)CC1] | 1136 |
| 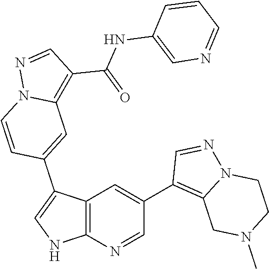 [SMILES: CN1CCn2ncc(-c3cnc4[nH]cc(-c5ccn6ncc(C(=O)Nc7cccnc7)c6c5)c4c3)c2C1] | 1137 |
| 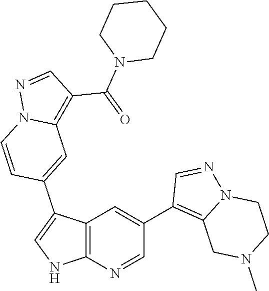 [SMILES: CN1CCn2ncc(-c3cnc4[nH]cc(-c5ccn6ncc(C(=O)N7CCCCC7)c6c5)c4c3)c2C1] | 1138 |
| 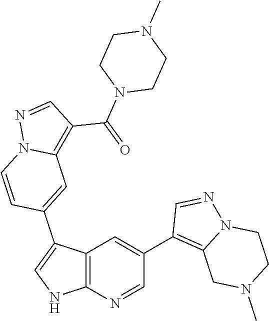 [SMILES: CN1CCN(C(=O)c2cnn3ccc(-c4c[nH]c5ncc(-c6cnn7c6CN(C)CC7)cc45)cc23)CC1] | 1139 |
| 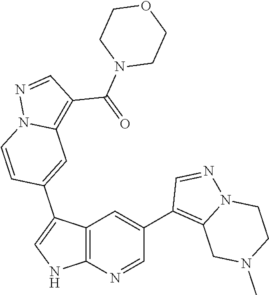 [SMILES: CN1CCn2ncc(-c3cnc4[nH]cc(-c5ccn6ncc(C(=O)N7CCOCC7)c6c5)c4c3)c2C1] | 1140 |
| 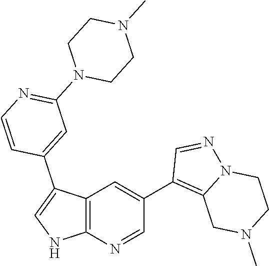 [SMILES: CN1CCN(c2cc(-c3c[nH]c4ncc(-c5cnn6c5CN(C)CC6)cc34)ccn2)CC1] | 1141 |
| 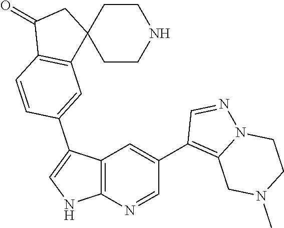 [SMILES: CN1CCn2ncc(-c3cnc4[nH]cc(-c5ccc6c(c5)C5(CCNCC5)CC6=O)c4c3)c2C1] | 1142 |
| 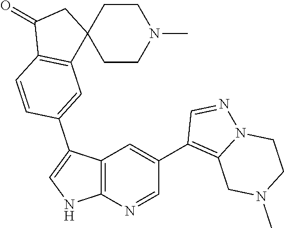 [SMILES: CN1CCC2(CC1)CC(=O)c1ccc(-c3c[nH]c4ncc(-c5cnn6c5CN(C)CC6)cc34)cc12] | 1143 |
| 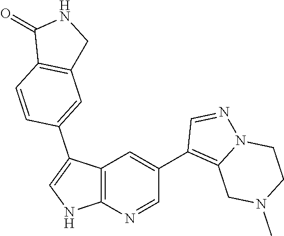 [SMILES: CN1CCn2ncc(-c3cnc4[nH]cc(-c5ccc6c(c5)CNC6=O)c4c3)c2C1] | 1144 |
| 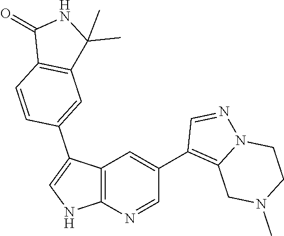 [SMILES: CN1CCn2ncc(-c3cnc4[nH]cc(-c5ccc6c(c5)C(C)(C)NC6=O)c4c3)c2C1] | 1145 |
| 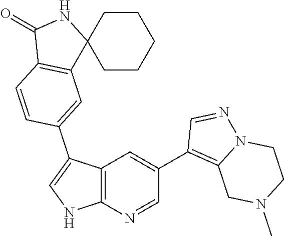 [SMILES: CN1CCn2ncc(-c3cnc4[nH]cc(-c5ccc6c(c5)C5(CCCCC5)NC6=O)c4c3)c2C1] | 1146 |
| 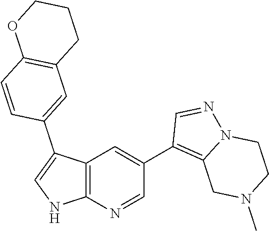 [SMILES: CN1CCn2ncc(-c3cnc4[nH]cc(-c5ccc6c(c5)CCCO6)c4c3)c2C1] | 1147 |
| 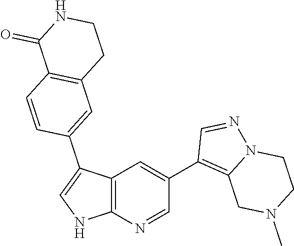 [SMILES: CN1CCn2ncc(-c3cnc4[nH]cc(-c5ccc6c(c5)CCNC6=O)c4c3)c2C1] | 1148 |
| 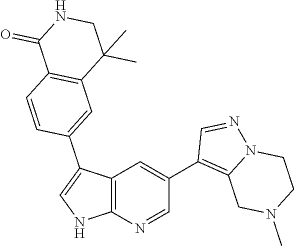 [SMILES: CN1CCn2ncc(-c3cnc4[nH]cc(-c5ccc6c(c5)C(C)(C)CNC6=O)c4c3)c2C1] | 1149 |
| 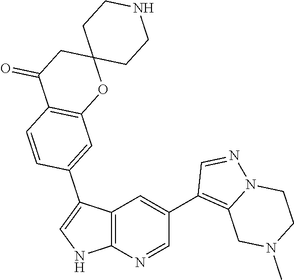 [SMILES: CN1CCn2ncc(-c3cnc4[nH]cc(-c5ccc6c(c5)OC5(CCNCC5)CC6=O)c4c3)c2C1] | 1150 |
| 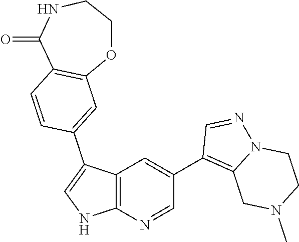 [SMILES: CN1CCn2ncc(-c3cnc4[nH]cc(-c5ccc6c(c5)OCCNC6=O)c4c3)c2C1] | 1151 |
| 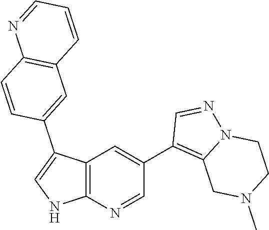 [SMILES: CN1CCn2ncc(-c3cnc4[nH]cc(-c5ccc6ncccc6c5)c4c3)c2C1] | 1152 |
| 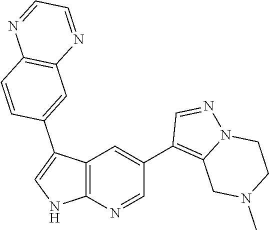 [SMILES: CN1CCn2ncc(-c3cnc4[nH]cc(-c5ccc6nccnc6c5)c4c3)c2C1] | 1153 |
| 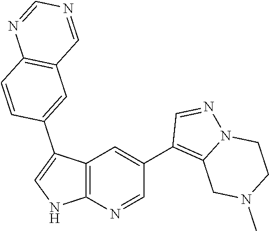 [SMILES: CN1CCn2ncc(-c3cnc4[nH]cc(-c5ccc6ncncc6c5)c4c3)c2C1] | 1154 |
| 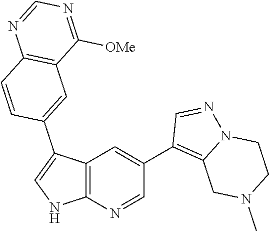 [SMILES: COc1ncnc2ccc(-c3c[nH]c4ncc(-c5cnn6c5CN(C)CC6)cc34)cc12] | 1155 |
| 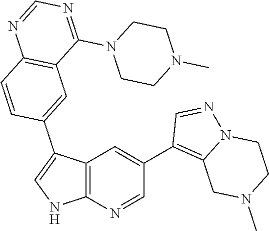 [SMILES: CN1CCN(c2ncnc3ccc(-c4c[nH]c5ncc(-c6cnn7c6CN(C)CC7)cc45)cc23)CC1] | 1156 |
| 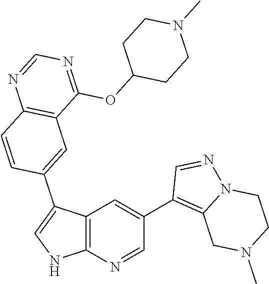 [SMILES: CN1CCC(Oc2ncnc3ccc(-c4c[nH]c5ncc(-c6cnn7c6CN(C)CC7)cc45)cc23)CC1] | 1157 |
| 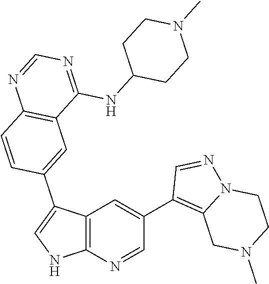 [SMILES: CN1CCC(Nc2ncnc3ccc(-c4c[nH]c5ncc(-c6cnn7c6CN(C)CC7)cc45)cc23)CC1] | 1158 |
| 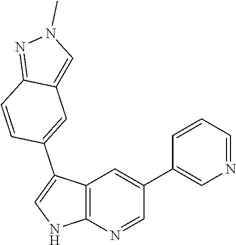 [SMILES: Cn1cc2cc(-c3c[nH]c4ncc(-c5cccnc5)cc34)ccc2n1] | 1159 |
| 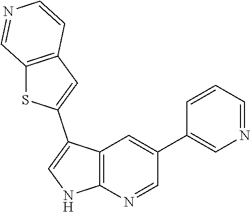 [SMILES: c1cncc(-c2cnc3[nH]cc(-c4cc5ccncc5s4)c3c2)c1] | 1160 |
| 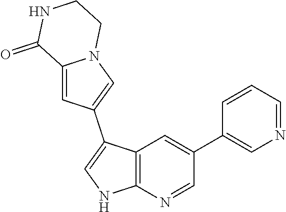 [SMILES: O=C1NCCn2cc(-c3c[nH]c4ncc(-c5cccnc5)cc34)cc21] | 1161 |
| 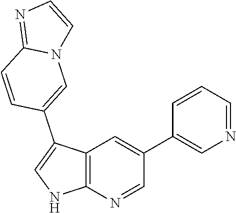 [SMILES: c1cncc(-c2cnc3[nH]cc(-c4ccc5nccn5c4)c3c2)c1] | 1162 |
| 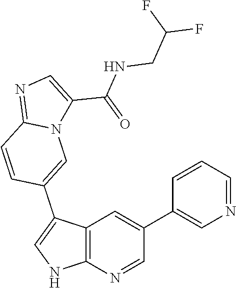 [SMILES: O=C(NCC(F)F)c1cnc2ccc(-c3c[nH]c4ncc(-c5cccnc5)cc34)cn12] | 1163 |
| 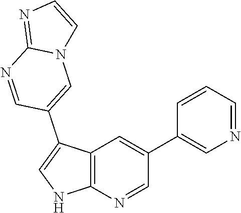 [SMILES: c1cncc(-c2cnc3[nH]cc(-c4cnc5nccn5c4)c3c2)c1] | 1164 |
| 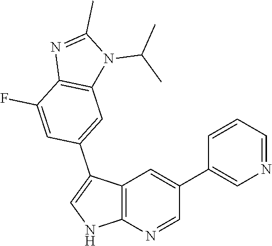 [SMILES: Cc1nc2c(F)cc(-c3c[nH]c4ncc(-c5cccnc5)cc34)cc2n1C(C)C] | 1165 |
| 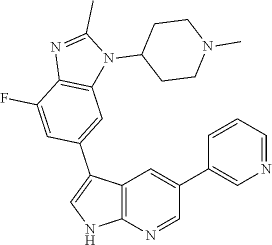 [SMILES: Cc1nc2c(F)cc(-c3c[nH]c4ncc(-c5cccnc5)cc34)cc2n1C1CCN(C)CC1] | 1166 |
| 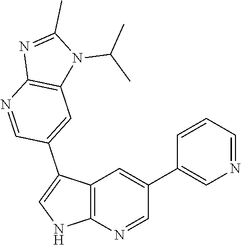 [SMILES: Cc1nc2ncc(-c3c[nH]c4ncc(-c5cccnc5)cc34)cc2n1C(C)C] | 1167 |
| 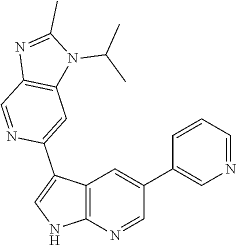 [SMILES: Cc1nc2cnc(-c3c[nH]c4ncc(-c5cccnc5)cc34)cc2n1C(C)C] | 1168 |
| 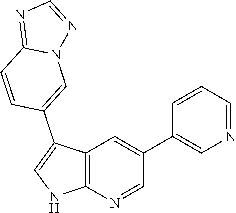 [SMILES: c1cncc(-c2cnc3[nH]cc(-c4ccc5ncnn5c4)c3c2)c1] | 1169 |
| 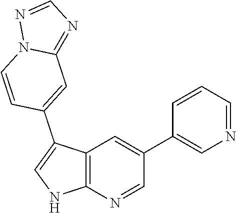 [SMILES: c1cncc(-c2cnc3[nH]cc(-c4ccn5ncnc5c4)c3c2)c1] | 1170 |
| 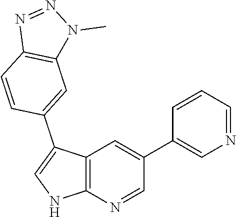 [SMILES: Cn1nnc2ccc(-c3c[nH]c4ncc(-c5cccnc5)cc34)cc21] | 1171 |
| 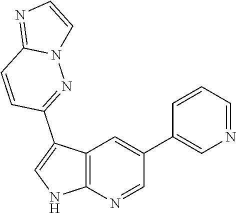 [SMILES: c1cncc(-c2cnc3[nH]cc(-c4ccc5nccn5n4)c3c2)c1] | 1172 |
| 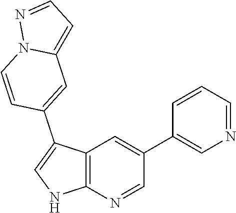 [SMILES: c1cncc(-c2cnc3[nH]cc(-c4ccn5nccc5c4)c3c2)c1] | 1173 |
| 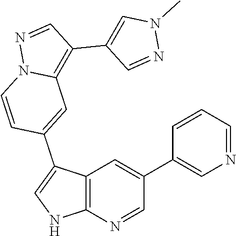 [SMILES: Cn1cc(-c2cnn3ccc(-c4c[nH]c5ncc(-c6cccnc6)cc45)cc23)cn1] | 1174 |
| 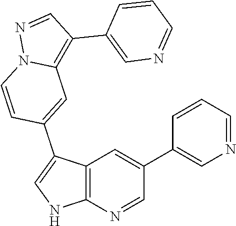 [SMILES: c1cncc(-c2cnc3[nH]cc(-c4ccn5ncc(-c6cccnc6)c5c4)c3c2)c1] | 1175 |
| 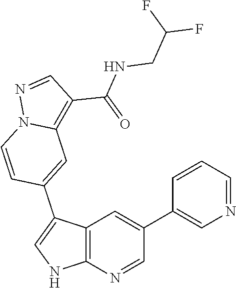 [SMILES: O=C(NCC(F)F)c1cnn2ccc(-c3c[nH]c4ncc(-c5cccnc5)cc34)cc12] | 1176 |
| 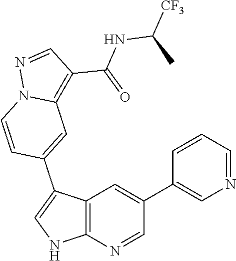 [SMILES: C[C@@H](NC(=O)c1cnn2ccc(-c3c[nH]c4ncc(-c5cccnc5)cc34)cc12)C(F)(F)F] | 1177 |
| 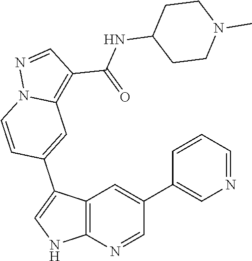 [SMILES: CN1CCC(NC(=O)c2cnn3ccc(-c4c[nH]c5ncc(-c6cccnc6)cc45)cc23)CC1] | 1178 |
| 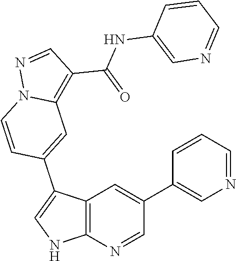 [SMILES: O=C(Nc1cccnc1)c1cnn2ccc(-c3c[nH]c4ncc(-c5cccnc5)cc34)cc12] | 1179 |
| 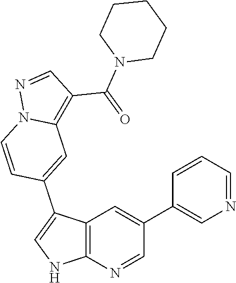 [SMILES: O=C(c1cnn2ccc(-c3c[nH]c4ncc(-c5cccnc5)cc34)cc12)N1CCCCC1] | 1180 |
| 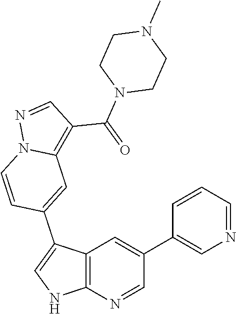 [SMILES: CN1CCN(C(=O)c2cnn3ccc(-c4c[nH]c5ncc(-c6cccnc6)cc45)cc23)CC1] | 1181 |
| 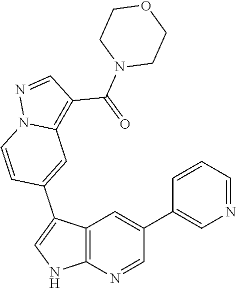 [SMILES: O=C(c1cnn2ccc(-c3c[nH]c4ncc(-c5cccnc5)cc34)cc12)N1CCOCC1] | 1182 |
| 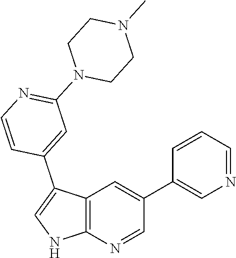 [SMILES: CN1CCN(c2cc(-c3c[nH]c4ncc(-c5cccnc5)cc34)ccn2)CC1] | 1183 |
| 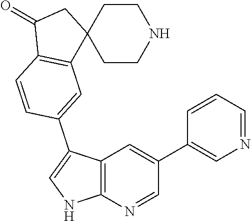 [SMILES: O=C1CC2(CCNCC2)c2cc(-c3c[nH]c4ncc(-c5cccnc5)cc34)ccc21] | 1184 |
| 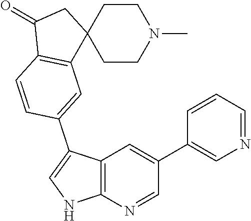 [SMILES: CN1CCC2(CC1)CC(=O)c1ccc(-c3c[nH]c4ncc(-c5cccnc5)cc34)cc12] | 1185 |
| 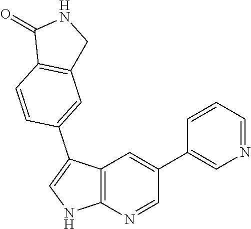 [SMILES: O=C1NCc2cc(-c3c[nH]c4ncc(-c5cccnc5)cc34)ccc21] | 1186 |
| 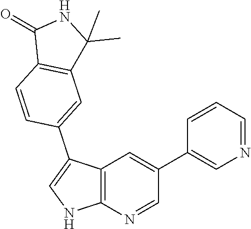 [SMILES: CC1(C)NC(=O)c2ccc(-c3c[nH]c4ncc(-c5cccnc5)cc34)cc21] | 1187 |
| 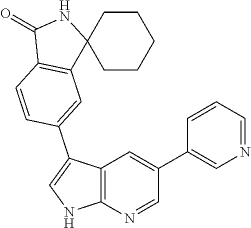 [SMILES: O=C1NC2(CCCCC2)c2cc(-c3c[nH]c4ncc(-c5cccnc5)cc34)ccc21] | 1188 |
| 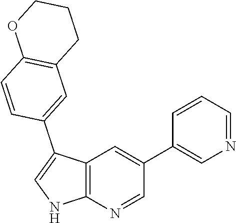 [SMILES: c1cncc(-c2cnc3[nH]cc(-c4ccc5c(c4)CCCO5)c3c2)c1] | 1189 |
| 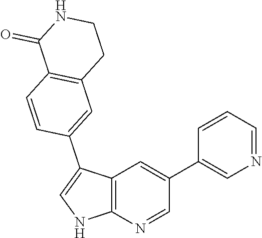 [SMILES: O=C1NCCc2cc(-c3c[nH]c4ncc(-c5cccnc5)cc34)ccc21] | 1190 |
| 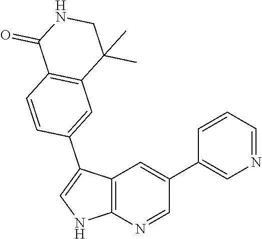 [SMILES: CC1(C)CNC(=O)c2ccc(-c3c[nH]c4ncc(-c5cccnc5)cc34)cc21] | 1191 |
| 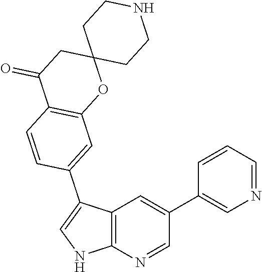 [SMILES: O=C1CC2(CCNCC2)Oc2cc(-c3c[nH]c4ncc(-c5cccnc5)cc34)ccc21] | 1192 |
| 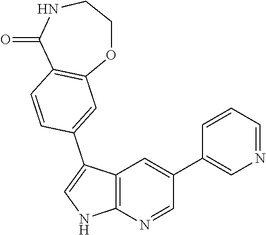 [SMILES: O=C1NCCOc2cc(-c3c[nH]c4ncc(-c5cccnc5)cc34)ccc21] | 1193 |
| 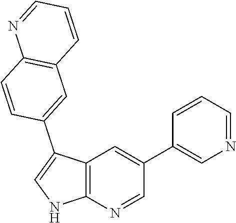 [SMILES: c1cncc(-c2cnc3[nH]cc(-c4ccc5ncccc5c4)c3c2)c1] | 1194 |
| 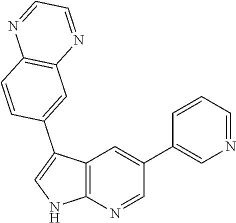 [SMILES: c1cncc(-c2cnc3[nH]cc(-c4ccc5nccnc5c4)c3c2)c1] | 1195 |
| 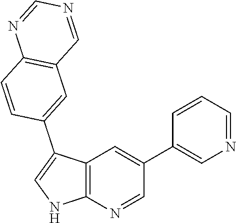 [SMILES: c1cncc(-c2cnc3[nH]cc(-c4ccc5ncncc5c4)c3c2)c1] | 1196 |
| 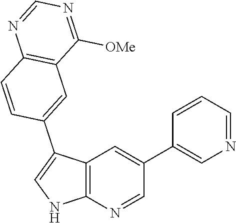 [SMILES: COc1ncnc2ccc(-c3c[nH]c4ncc(-c5cccnc5)cc34)cc12] | 1197 |
| 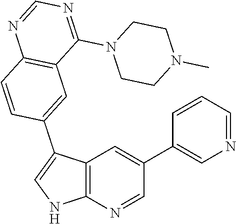 [SMILES: CN1CCN(c2ncnc3ccc(-c4c[nH]c5ncc(-c6cccnc6)cc45)cc23)CC1] | 1198 |
| 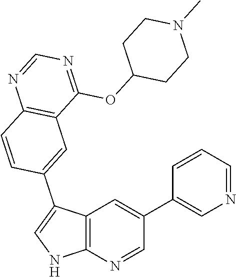 [SMILES: CN1CCC(Oc2ncnc3ccc(-c4c[nH]c5ncc(-c6cccnc6)cc45)cc23)CC1] | 1199 |
| 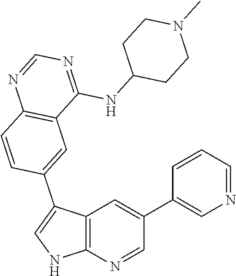 [SMILES: CN1CCC(Nc2ncnc3ccc(-c4c[nH]c5ncc(-c6cccnc6)cc45)cc23)CC1] | 1200 |
| 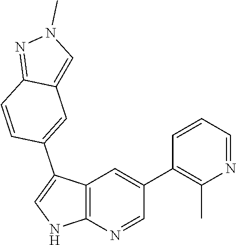 [SMILES: Cc1ncccc1-c1cnc2[nH]cc(-c3ccc4nn(C)cc4c3)c2c1] | 1201 |
| 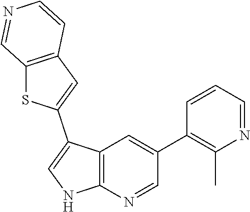 [SMILES: Cc1ncccc1-c1cnc2[nH]cc(-c3cc4ccncc4s3)c2c1] | 1202 |
| 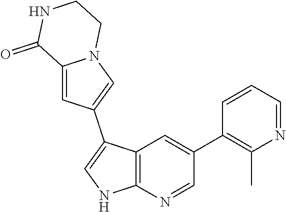 [SMILES: Cc1ncccc1-c1cnc2[nH]cc(-c3cc4n(c3)CCNC4=O)c2c1] | 1203 |
| 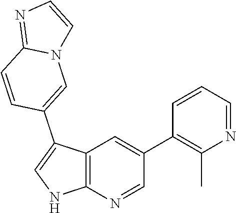 [SMILES: Cc1ncccc1-c1cnc2[nH]cc(-c3ccc4nccn4c3)c2c1] | 1204 |
| 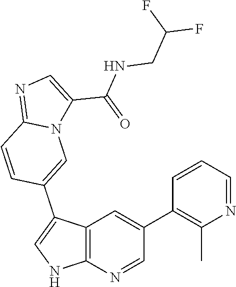 [SMILES: Cc1ncccc1-c1cnc2[nH]cc(-c3ccc4ncc(C(=O)NCC(F)F)n4c3)c2c1] | 1205 |
| 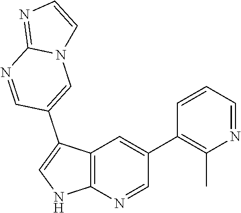 [SMILES: Cc1ncccc1-c1cnc2[nH]cc(-c3cnc4nccn4c3)c2c1] | 1206 |
| 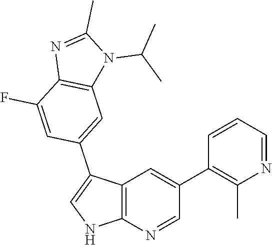 [SMILES: Cc1ncccc1-c1cnc2[nH]cc(-c3cc(F)c4nc(C)n(C(C)C)c4c3)c2c1] | 1207 |
| 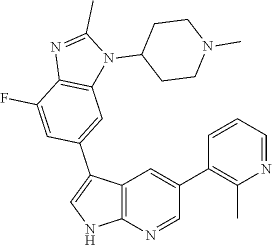 [SMILES: Cc1ncccc1-c1cnc2[nH]cc(-c3cc(F)c4nc(C)n(C5CCN(C)CC5)c4c3)c2c1] | 1208 |
| 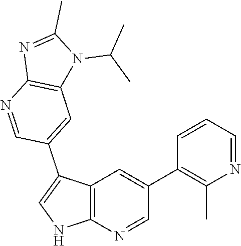 [SMILES: Cc1ncccc1-c1cnc2[nH]cc(-c3cnc4nc(C)n(C(C)C)c4c3)c2c1] | 1209 |
| 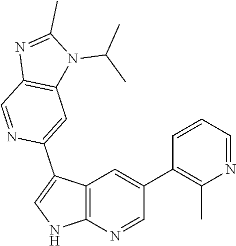 [SMILES: Cc1ncccc1-c1cnc2[nH]cc(-c3cc4c(cn3)nc(C)n4C(C)C)c2c1] | 1210 |
| 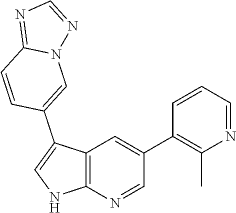 [SMILES: Cc1ncccc1-c1cnc2[nH]cc(-c3ccc4ncnn4c3)c2c1] | 1211 |
| 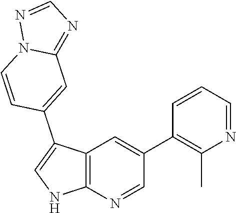 [SMILES: Cc1ncccc1-c1cnc2[nH]cc(-c3ccn4ncnc4c3)c2c1] | 1212 |
| 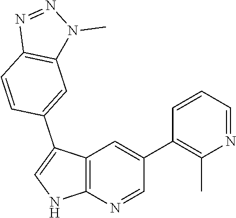 [SMILES: Cc1ncccc1-c1cnc2[nH]cc(-c3ccc4nnn(C)c4c3)c2c1] | 1213 |
| 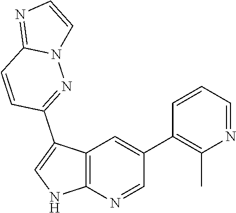 [SMILES: Cc1ncccc1-c1cnc2[nH]cc(-c3ccc4nccn4n3)c2c1] | 1214 |
| 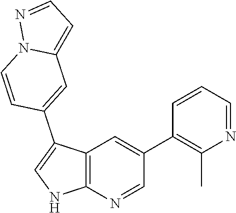 [SMILES: Cc1ncccc1-c1cnc2[nH]cc(-c3ccn4nccc4c3)c2c1] | 1215 |
| 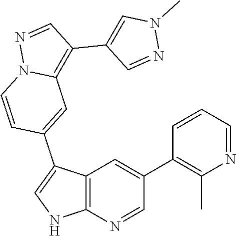 [SMILES: Cc1ncccc1-c1cnc2[nH]cc(-c3ccn4ncc(-c5cnn(C)c5)c4c3)c2c1] | 1216 |
| 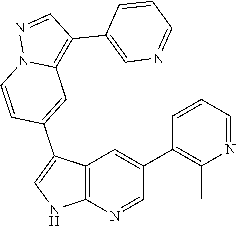 [SMILES: Cc1ncccc1-c1cnc2[nH]cc(-c3ccn4ncc(-c5cccnc5)c4c3)c2c1] | 1217 |
| 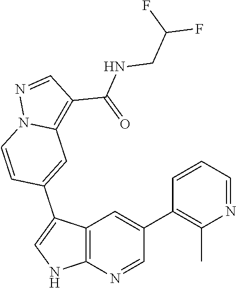 [SMILES: Cc1ncccc1-c1cnc2[nH]cc(-c3ccn4ncc(C(=O)NCC(F)F)c4c3)c2c1] | 1218 |
| 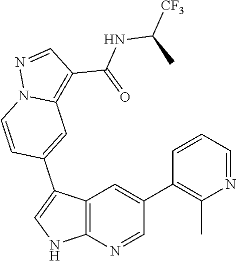 [SMILES: Cc1ncccc1-c1cnc2[nH]cc(-c3ccn4ncc(C(=O)N[C@H](C)C(F)(F)F)c4c3)c2c1] | 1219 |
| 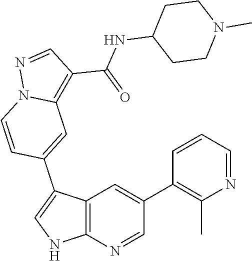 [SMILES: Cc1ncccc1-c1cnc2[nH]cc(-c3ccn4ncc(C(=O)NC5CCN(C)CC5)c4c3)c2c1] | 1220 |
| 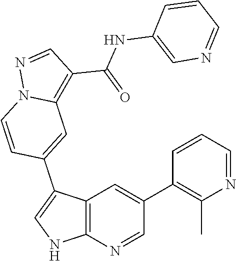 [SMILES: Cc1ncccc1-c1cnc2[nH]cc(-c3ccn4ncc(C(=O)Nc5cccnc5)c4c3)c2c1] | 1221 |
| 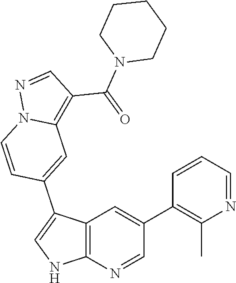 [SMILES: Cc1ncccc1-c1cnc2[nH]cc(-c3ccn4ncc(C(=O)N5CCCCC5)c4c3)c2c1] | 1222 |
| 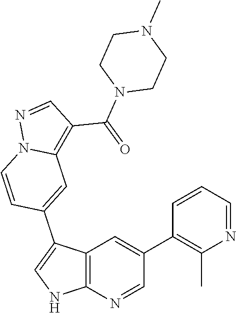 [SMILES: Cc1ncccc1-c1cnc2[nH]cc(-c3ccn4ncc(C(=O)N5CCN(C)CC5)c4c3)c2c1] | 1223 |
| 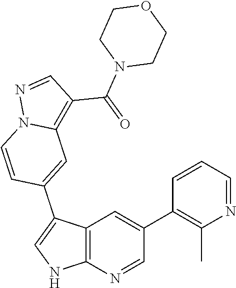 [SMILES: Cc1ncccc1-c1cnc2[nH]cc(-c3ccn4ncc(C(=O)N5CCOCC5)c4c3)c2c1] | 1224 |
| 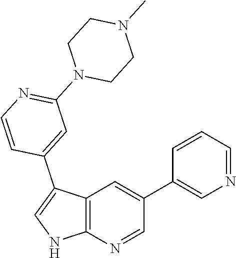 [SMILES: CN1CCN(c2cc(-c3c[nH]c4ncc(-c5cccnc5)cc34)ccn2)CC1] | 1225 |
| 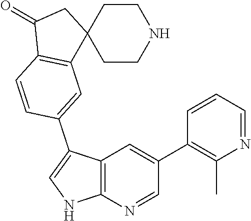 [SMILES: Cc1ncccc1-c1cnc2[nH]cc(-c3ccc4c(c3)C3(CCNCC3)CC4=O)c2c1] | 1226 |
| 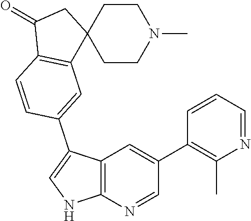 [SMILES: Cc1ncccc1-c1cnc2[nH]cc(-c3ccc4c(c3)C3(CCN(C)CC3)CC4=O)c2c1] | 1227 |
| 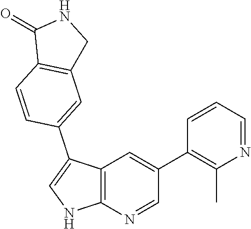 [SMILES: Cc1ncccc1-c1cnc2[nH]cc(-c3ccc4c(c3)CNC4=O)c2c1] | 1228 |
| 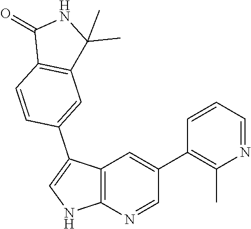 [SMILES: Cc1ncccc1-c1cnc2[nH]cc(-c3ccc4c(c3)C(C)(C)NC4=O)c2c1] | 1229 |
| 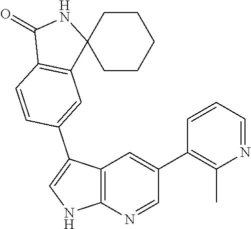 [SMILES: Cc1ncccc1-c1cnc2[nH]cc(-c3ccc4c(c3)C3(CCCCC3)NC4=O)c2c1] | 1230 |
| 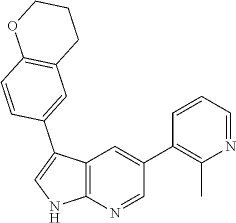 [SMILES: Cc1ncccc1-c1cnc2[nH]cc(-c3ccc4c(c3)CCCO4)c2c1] | 1231 |
| 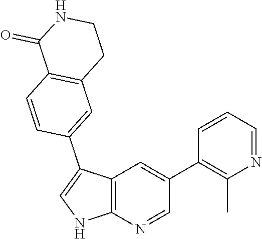 [SMILES: Cc1ncccc1-c1cnc2[nH]cc(-c3ccc4c(c3)CCNC4=O)c2c1] | 1232 |
| 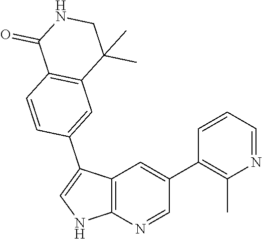 [SMILES: Cc1ncccc1-c1cnc2[nH]cc(-c3ccc4c(c3)C(C)(C)CNC4=O)c2c1] | 1233 |
| 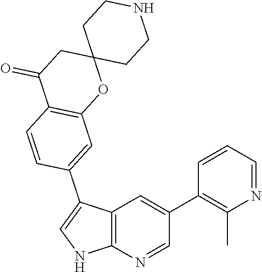 [SMILES: Cc1ncccc1-c1cnc2[nH]cc(-c3ccc4c(c3)OC3(CCNCC3)CC4=O)c2c1] | 1234 |
| 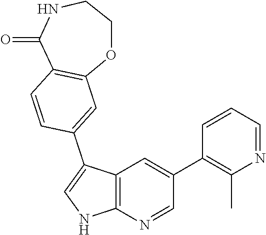 [SMILES: Cc1ncccc1-c1cnc2[nH]cc(-c3ccc4c(c3)OCCNC4=O)c2c1] | 1235 |
| 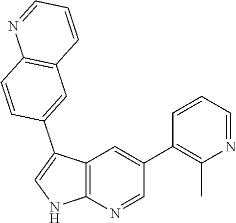 [SMILES: Cc1ncccc1-c1cnc2[nH]cc(-c3ccc4ncccc4c3)c2c1] | 1236 |
| 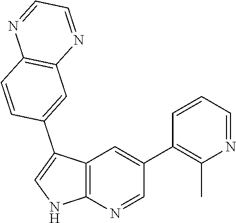 [SMILES: Cc1ncccc1-c1cnc2[nH]cc(-c3ccc4nccnc4c3)c2c1] | 1237 |
| 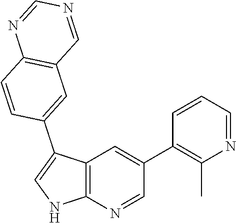 [SMILES: Cc1ncccc1-c1cnc2[nH]cc(-c3ccc4ncncc4c3)c2c1] | 1238 |
| 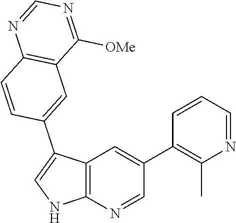 [SMILES: COc1ncnc2ccc(-c3c[nH]c4ncc(-c5cccnc5C)cc34)cc12] | 1239 |
| 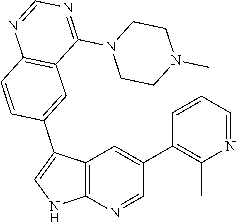 [SMILES: Cc1ncccc1-c1cnc2[nH]cc(-c3ccc4ncnc(N5CCN(C)CC5)c4c3)c2c1] | 1240 |
| 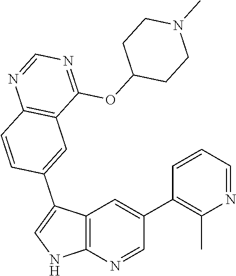 [SMILES: Cc1ncccc1-c1cnc2[nH]cc(-c3ccc4ncnc(OC5CCN(C)CC5)c4c3)c2c1] | 1241 |
| 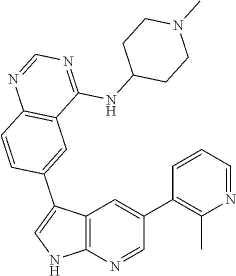 [SMILES: Cc1ncccc1-c1cnc2[nH]cc(-c3ccc4ncnc(NC5CCN(C)CC5)c4c3)c2c1] | 1242 |
| 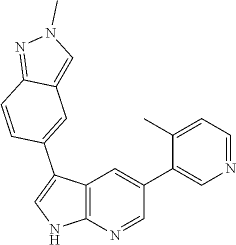 [SMILES: Cc1ccncc1-c1cnc2[nH]cc(-c3ccc4nn(C)cc4c3)c2c1] | 1243 |
| 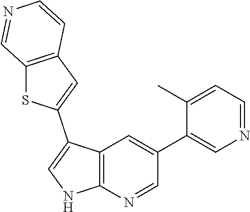 [SMILES: Cc1ccncc1-c1cnc2[nH]cc(-c3cc4ccncc4s3)c2c1] | 1244 |
| 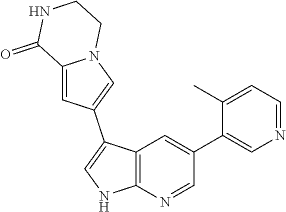 [SMILES: Cc1ccncc1-c1cnc2[nH]cc(-c3cc4n(c3)CCNC4=O)c2c1] | 1245 |
| 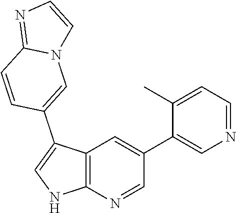 [SMILES: Cc1ccncc1-c1cnc2[nH]cc(-c3ccc4nccn4c3)c2c1] | 1246 |
| 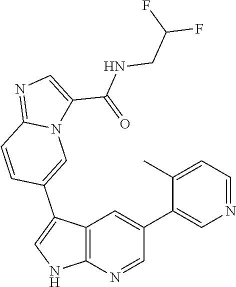 [SMILES: Cc1ccncc1-c1cnc2[nH]cc(-c3ccc4ncc(C(=O)NCC(F)F)n4c3)c2c1] | 1247 |
| 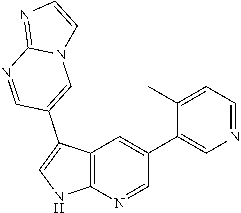 [SMILES: Cc1ccncc1-c1cnc2[nH]cc(-c3cnc4nccn4c3)c2c1] | 1248 |
| 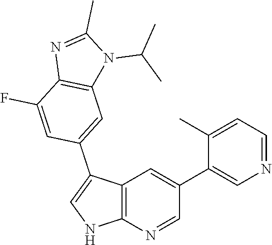 [SMILES: Cc1ccncc1-c1cnc2[nH]cc(-c3cc(F)c4nc(C)n(C(C)C)c4c3)c2c1] | 1249 |
| 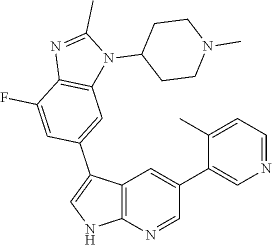 [SMILES: Cc1ccncc1-c1cnc2[nH]cc(-c3cc(F)c4nc(C)n(C5CCN(C)CC5)c4c3)c2c1] | 1250 |
| 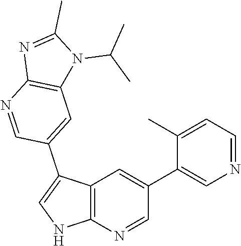 [SMILES: Cc1ccncc1-c1cnc2[nH]cc(-c3cnc4nc(C)n(C(C)C)c4c3)c2c1] | 1251 |
| 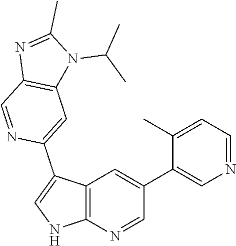 [SMILES: Cc1ccncc1-c1cnc2[nH]cc(-c3cc4c(cn3)nc(C)n4C(C)C)c2c1] | 1252 |
| 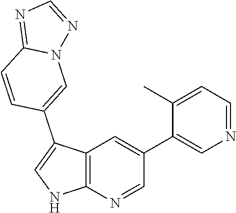 [SMILES: Cc1ccncc1-c1cnc2[nH]cc(-c3ccc4ncnn4c3)c2c1] | 1253 |
| 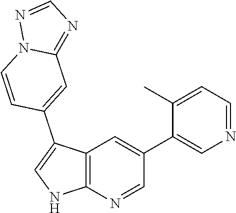 [SMILES: Cc1ccncc1-c1cnc2[nH]cc(-c3ccn4ncnc4c3)c2c1] | 1254 |
| 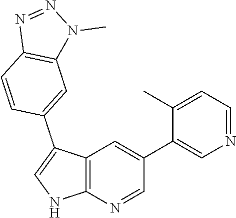 [SMILES: Cc1ccncc1-c1cnc2[nH]cc(-c3ccc4nnn(C)c4c3)c2c1] | 1255 |
| 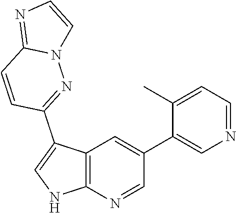 [SMILES: Cc1ccncc1-c1cnc2[nH]cc(-c3ccc4nccn4n3)c2c1] | 1256 |
| 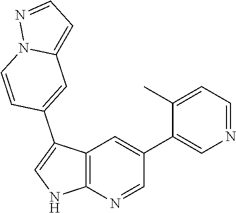 [SMILES: Cc1ccncc1-c1cnc2[nH]cc(-c3ccn4nccc4c3)c2c1] | 1257 |
| 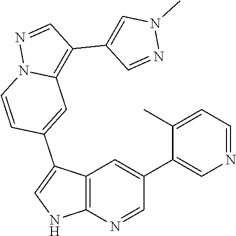 [SMILES: Cc1ccncc1-c1cnc2[nH]cc(-c3ccn4ncc(-c5cnn(C)c5)c4c3)c2c1] | 1258 |
| 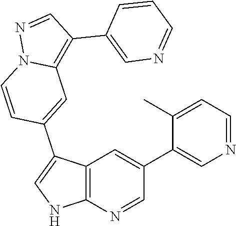 [SMILES: Cc1ccncc1-c1cnc2[nH]cc(-c3ccn4ncc(-c5cccnc5)c4c3)c2c1] | 1259 |
| 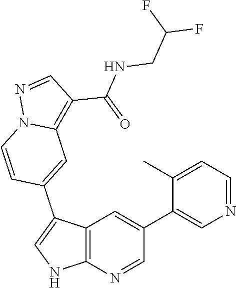 [SMILES: Cc1ccncc1-c1cnc2[nH]cc(-c3ccn4ncc(C(=O)NCC(F)F)c4c3)c2c1] | 1260 |
| 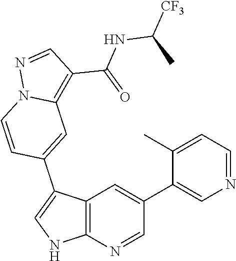 [SMILES: Cc1ccncc1-c1cnc2[nH]cc(-c3ccn4ncc(C(=O)N[C@H](C)C(F)(F)F)c4c3)c2c1] | 1261 |
| 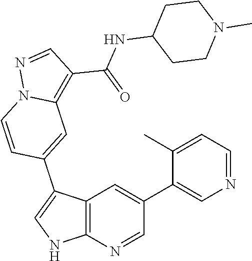 [SMILES: Cc1ccncc1-c1cnc2[nH]cc(-c3ccn4ncc(C(=O)NC5CCN(C)CC5)c4c3)c2c1] | 1262 |
| 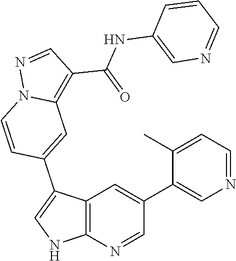 [SMILES: Cc1ccncc1-c1cnc2[nH]cc(-c3ccn4ncc(C(=O)Nc5cccnc5)c4c3)c2c1] | 1263 |
| 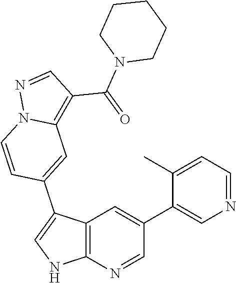 [SMILES: Cc1ccncc1-c1cnc2[nH]cc(-c3ccn4ncc(C(=O)N5CCCCC5)c4c3)c2c1] | 1264 |
| 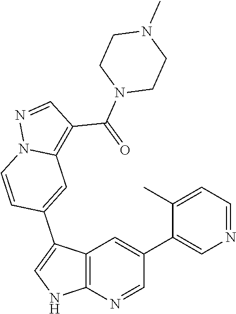 [SMILES: Cc1ccncc1-c1cnc2[nH]cc(-c3ccn4ncc(C(=O)N5CCN(C)CC5)c4c3)c2c1] | 1265 |
| 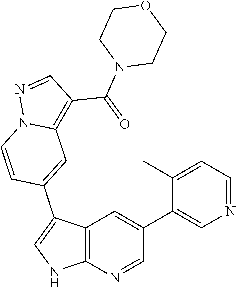 [SMILES: Cc1ccncc1-c1cnc2[nH]cc(-c3ccn4ncc(C(=O)N5CCOCC5)c4c3)c2c1] | 1266 |
| 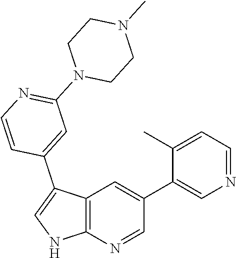 [SMILES: Cc1ccncc1-c1cnc2[nH]cc(-c3ccnc(N4CCN(C)CC4)c3)c2c1] | 1267 |
| 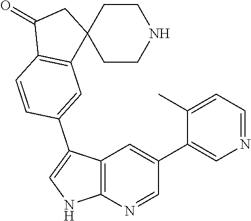 [SMILES: Cc1ccncc1-c1cnc2[nH]cc(-c3ccc4c(c3)C3(CCNCC3)CC4=O)c2c1] | 1268 |
| 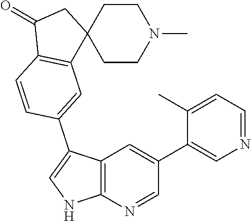 [SMILES: Cc1ccncc1-c1cnc2[nH]cc(-c3ccc4c(c3)C3(CCN(C)CC3)CC4=O)c2c1] | 1269 |
| 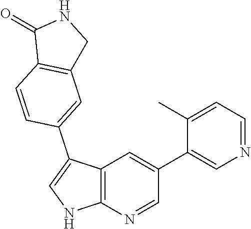 [SMILES: Cc1ccncc1-c1cnc2[nH]cc(-c3ccc4c(c3)CNC4=O)c2c1] | 1270 |
| 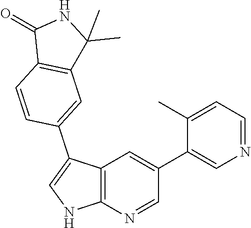 [SMILES: Cc1ccncc1-c1cnc2[nH]cc(-c3ccc4c(c3)C(C)(C)NC4=O)c2c1] | 1271 |
| 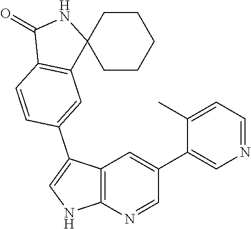 [SMILES: Cc1ccncc1-c1cnc2[nH]cc(-c3ccc4c(c3)C3(CCCCC3)NC4=O)c2c1] | 1272 |
| 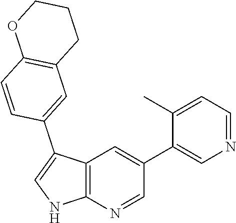 [SMILES: Cc1ccncc1-c1cnc2[nH]cc(-c3ccc4c(c3)CCCO4)c2c1] | 1273 |
| 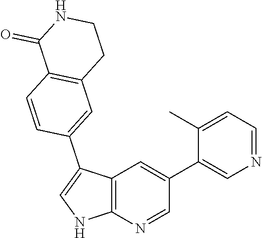 [SMILES: Cc1ccncc1-c1cnc2[nH]cc(-c3ccc4c(c3)CCNC4=O)c2c1] | 1274 |
| 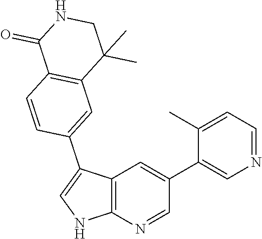 [SMILES: Cc1ccncc1-c1cnc2[nH]cc(-c3ccc4c(c3)C(C)(C)CNC4=O)c2c1] | 1275 |
| 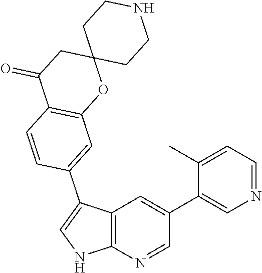 [SMILES: Cc1ccncc1-c1cnc2[nH]cc(-c3ccc4c(c3)OC3(CCNCC3)CC4=O)c2c1] | 1276 |
| 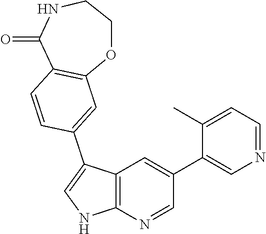 [SMILES: Cc1ccncc1-c1cnc2[nH]cc(-c3ccc4c(c3)OCCNC4=O)c2c1] | 1277 |
| 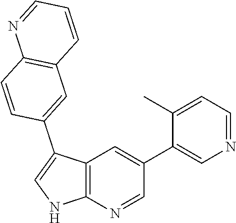 [SMILES: Cc1ccncc1-c1cnc2[nH]cc(-c3ccc4ncccc4c3)c2c1] | 1278 |
| 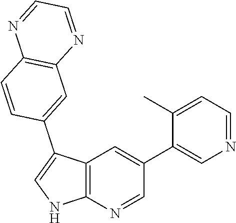 [SMILES: Cc1ccncc1-c1cnc2[nH]cc(-c3ccc4nccnc4c3)c2c1] | 1279 |
| 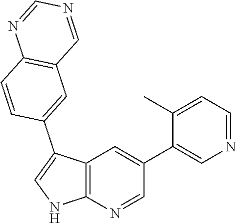 [SMILES: Cc1ccncc1-c1cnc2[nH]cc(-c3ccc4ncncc4c3)c2c1] | 1280 |
| 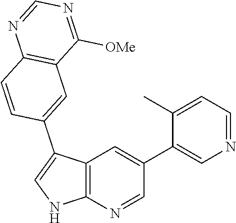 [SMILES: COc1ncnc2ccc(-c3c[nH]c4ncc(-c5cnccc5C)cc34)cc12] | 1281 |
| 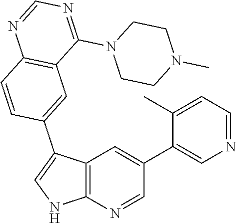 [SMILES: Cc1ccncc1-c1cnc2[nH]cc(-c3ccc4ncnc(N5CCN(C)CC5)c4c3)c2c1] | 1282 |
| 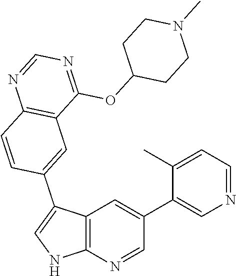 [SMILES: Cc1ccncc1-c1cnc2[nH]cc(-c3ccc4ncnc(OC5CCN(C)CC5)c4c3)c2c1] | 1283 |
| 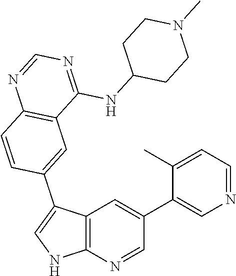 [SMILES: Cc1ccncc1-c1cnc2[nH]cc(-c3ccc4ncnc(NC5CCN(C)CC5)c4c3)c2c1] | 1284 |
| 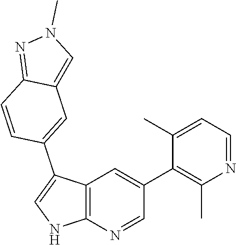 [SMILES: Cc1ccnc(C)c1-c1cnc2[nH]cc(-c3ccc4nn(C)cc4c3)c2c1] | 1285 |
| 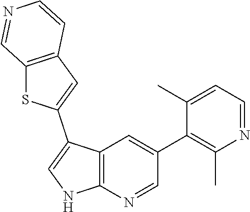 [SMILES: Cc1ccnc(C)c1-c1cnc2[nH]cc(-c3cc4ccncc4s3)c2c1] | 1286 |
| 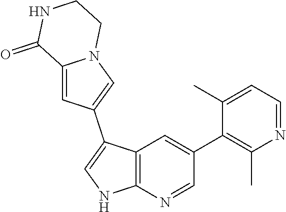 [SMILES: Cc1ccnc(C)c1-c1cnc2[nH]cc(-c3cc4n(c3)CCNC4=O)c2c1] | 1287 |
| 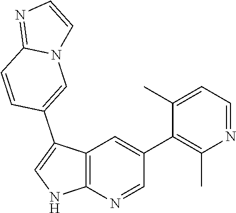 [SMILES: Cc1ccnc(C)c1-c1cnc2[nH]cc(-c3ccc4nccn4c3)c2c1] | 1288 |
| 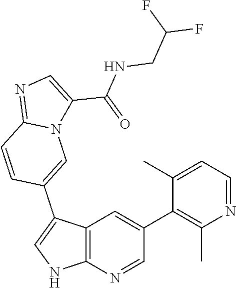 [SMILES: Cc1ccnc(C)c1-c1cnc2[nH]cc(-c3ccc4ncc(C(=O)NCC(F)F)n4c3)c2c1] | 1289 |
| 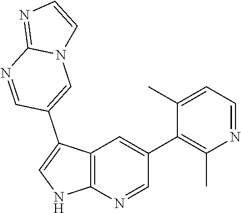 [SMILES: Cc1ccnc(C)c1-c1cnc2[nH]cc(-c3cnc4nccn4c3)c2c1] | 1290 |
| 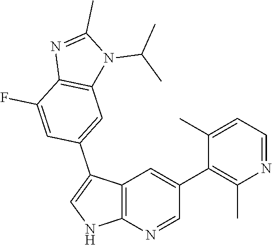 [SMILES: Cc1ccnc(C)c1-c1cnc2[nH]cc(-c3cc(F)c4nc(C)n(C(C)C)c4c3)c2c1] | 1291 |
| 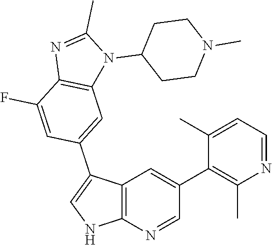 [SMILES: Cc1ccnc(C)c1-c1cnc2[nH]cc(-c3cc(F)c4nc(C)n(C5CCN(C)CC5)c4c3)c2c1] | 1292 |
| 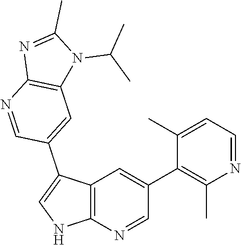 [SMILES: Cc1ccnc(C)c1-c1cnc2[nH]cc(-c3cnc4nc(C)n(C(C)C)c4c3)c2c1] | 1293 |
| 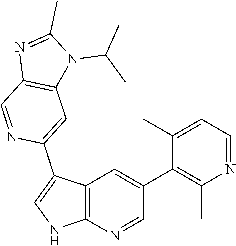 [SMILES: Cc1ccnc(C)c1-c1cnc2[nH]cc(-c3cc4c(cn3)nc(C)n4C(C)C)c2c1] | 1294 |
| 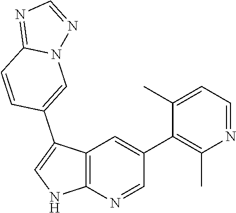 [SMILES: Cc1ccnc(C)c1-c1cnc2[nH]cc(-c3ccc4ncnn4c3)c2c1] | 1295 |
| 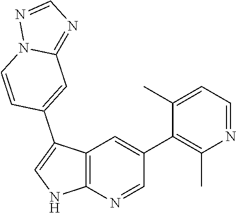 [SMILES: Cc1ccnc(C)c1-c1cnc2[nH]cc(-c3ccn4ncnc4c3)c2c1] | 1296 |
| 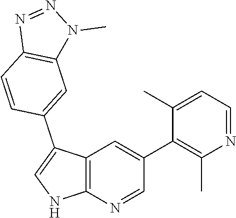 [SMILES: Cc1ccnc(C)c1-c1cnc2[nH]cc(-c3ccc4nnn(C)c4c3)c2c1] | 1297 |
| 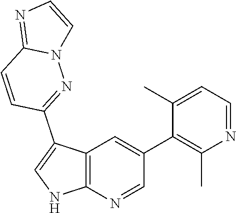 [SMILES: Cc1ccnc(C)c1-c1cnc2[nH]cc(-c3ccc4nccn4n3)c2c1] | 1298 |
| 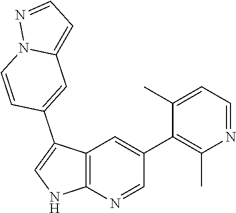 [SMILES: Cc1ccnc(C)c1-c1cnc2[nH]cc(-c3ccn4nccc4c3)c2c1] | 1299 |
| 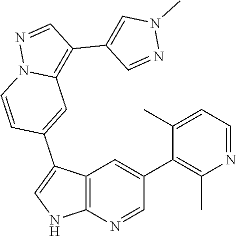 [SMILES: Cc1ccnc(C)c1-c1cnc2[nH]cc(-c3ccn4ncc(-c5cnn(C)c5)c4c3)c2c1] | 1300 |
| 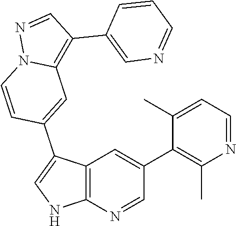 [SMILES: Cc1ccnc(C)c1-c1cnc2[nH]cc(-c3ccn4ncc(-c5cccnc5)c4c3)c2c1] | 1301 |
| 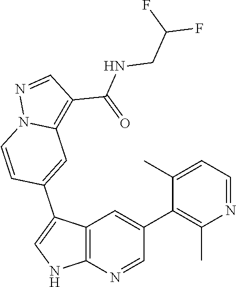 [SMILES: Cc1ccnc(C)c1-c1cnc2[nH]cc(-c3ccn4ncc(C(=O)NCC(F)F)c4c3)c2c1] | 1302 |
| 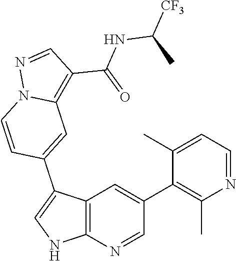 [SMILES: Cc1ccnc(C)c1-c1cnc2[nH]cc(-c3ccn4ncc(C(=O)N[C@H](C)C(F)(F)F)c4c3)c2c1] | 1303 |
| 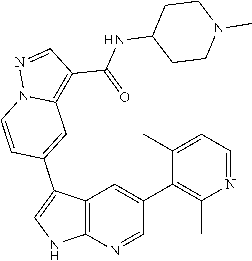 [SMILES: Cc1ccnc(C)c1-c1cnc2[nH]cc(-c3ccn4ncc(C(=O)NC5CCN(C)CC5)c4c3)c2c1] | 1304 |
| 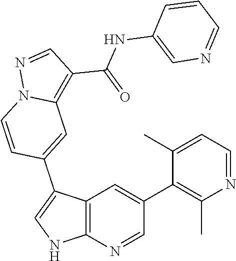 [SMILES: Cc1ccnc(C)c1-c1cnc2[nH]cc(-c3ccn4ncc(C(=O)Nc5cccnc5)c4c3)c2c1] | 1305 |
| 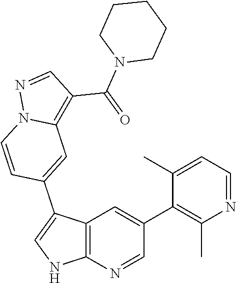 [SMILES: Cc1ccnc(C)c1-c1cnc2[nH]cc(-c3ccn4ncc(C(=O)N5CCCCC5)c4c3)c2c1] | 1306 |
| 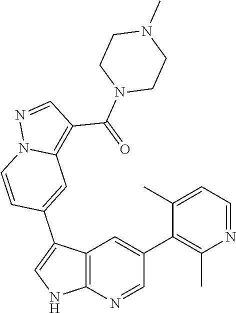 [SMILES: Cc1ccnc(C)c1-c1cnc2[nH]cc(-c3ccn4ncc(C(=O)N5CCN(C)CC5)c4c3)c2c1] | 1307 |
| 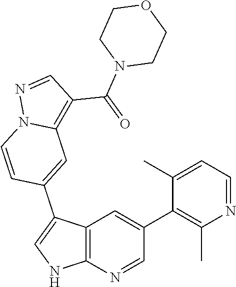 [SMILES: Cc1ccnc(C)c1-c1cnc2[nH]cc(-c3ccn4ncc(C(=O)N5CCOCC5)c4c3)c2c1] | 1308 |
| 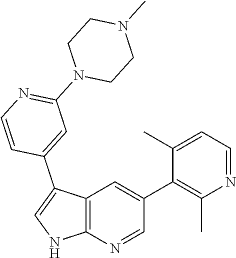 [SMILES: Cc1ccnc(C)c1-c1cnc2[nH]cc(-c3ccnc(N4CCN(C)CC4)c3)c2c1] | 1309 |
| 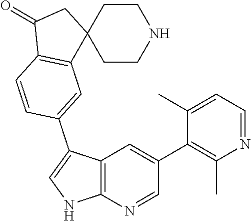 [SMILES: Cc1ccnc(C)c1-c1cnc2[nH]cc(-c3ccc4c(c3)C3(CCNCC3)CC4=O)c2c1] | 1310 |
| 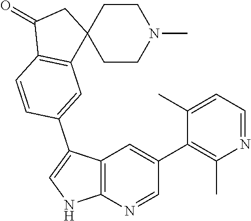 [SMILES: Cc1ccnc(C)c1-c1cnc2[nH]cc(-c3ccc4c(c3)C3(CCN(C)CC3)CC4=O)c2c1] | 1311 |
| 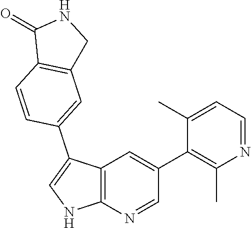 [SMILES: Cc1ccnc(C)c1-c1cnc2[nH]cc(-c3ccc4c(c3)CNC4=O)c2c1] | 1312 |
| 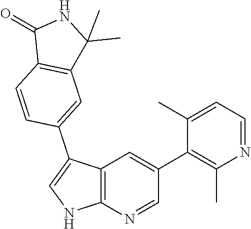 [SMILES: Cc1ccnc(C)c1-c1cnc2[nH]cc(-c3ccc4c(c3)C(C)(C)NC4=O)c2c1] | 1313 |
| 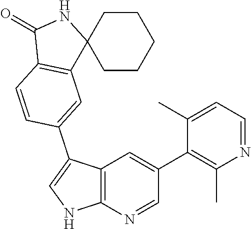 [SMILES: Cc1ccnc(C)c1-c1cnc2[nH]cc(-c3ccc4c(c3)C3(CCCCC3)NC4=O)c2c1] | 1314 |
| 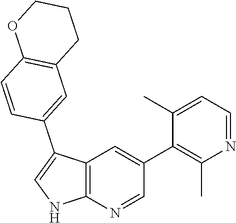 [SMILES: Cc1ccnc(C)c1-c1cnc2[nH]cc(-c3ccc4c(c3)CCCO4)c2c1] | 1315 |
| 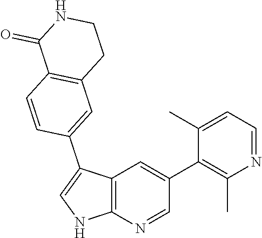 [SMILES: Cc1ccnc(C)c1-c1cnc2[nH]cc(-c3ccc4c(c3)CCNC4=O)c2c1] | 1316 |
| 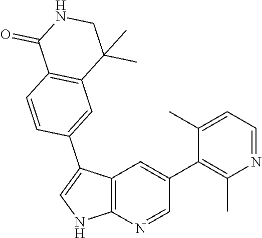 [SMILES: Cc1ccnc(C)c1-c1cnc2[nH]cc(-c3ccc4c(c3)C(C)(C)CNC4=O)c2c1] | 1317 |
| 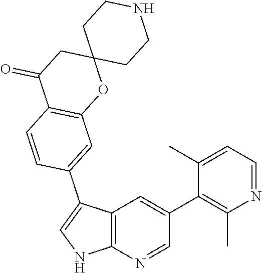 [SMILES: Cc1ccnc(C)c1-c1cnc2[nH]cc(-c3ccc4c(c3)OC3(CCNCC3)CC4=O)c2c1] | 1318 |
| 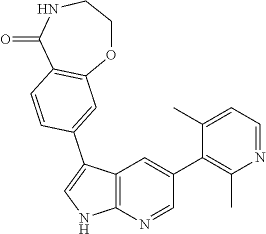 [SMILES: Cc1ccnc(C)c1-c1cnc2[nH]cc(-c3ccc4c(c3)OCCNC4=O)c2c1] | 1319 |
| 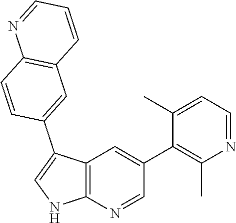 [SMILES: Cc1ccnc(C)c1-c1cnc2[nH]cc(-c3ccc4ncccc4c3)c2c1] | 1320 |
| 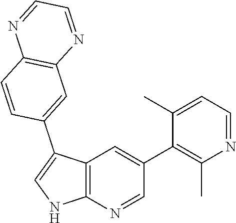 [SMILES: Cc1ccnc(C)c1-c1cnc2[nH]cc(-c3ccc4nccnc4c3)c2c1] | 1321 |
| 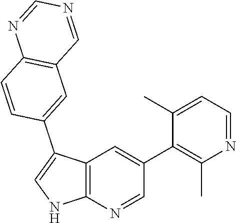 [SMILES: Cc1ccnc(C)c1-c1cnc2[nH]cc(-c3ccc4ncncc4c3)c2c1] | 1322 |
| 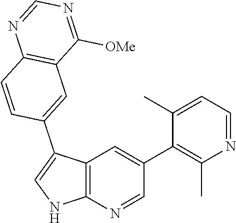 [SMILES: COc1ncnc2ccc(-c3c[nH]c4ncc(-c5c(C)ccnc5C)cc34)cc12] | 1323 |
| 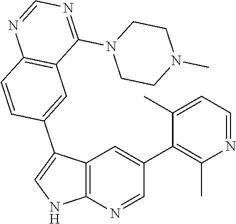 [SMILES: Cc1ccnc(C)c1-c1cnc2[nH]cc(-c3ccc4ncnc(N5CCN(C)CC5)c4c3)c2c1] | 1324 |
| 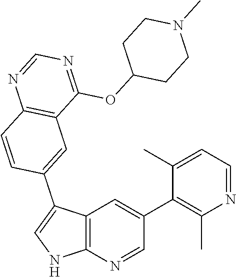 [SMILES: Cc1ccnc(C)c1-c1cnc2[nH]cc(-c3ccc4ncnc(OC5CCN(C)CC5)c4c3)c2c1] | 1325 |
| 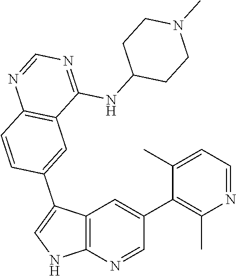 [SMILES: Cc1ccnc(C)c1-c1cnc2[nH]cc(-c3ccc4ncnc(NC5CCN(C)CC5)c4c3)c2c1] | 1326 |
| 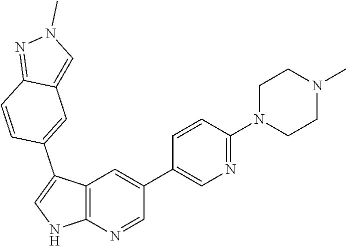 [SMILES: CN1CCN(c2ccc(-c3cnc4[nH]cc(-c5ccc6nn(C)cc6c5)c4c3)cn2)CC1] | 1327 |
| 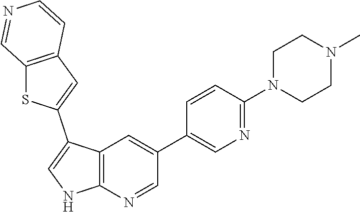 [SMILES: CN1CCN(c2ccc(-c3cnc4[nH]cc(-c5cc6ccncc6s5)c4c3)cn2)CC1] | 1328 |
| 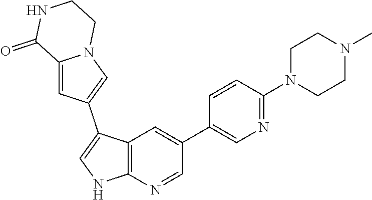 [SMILES: CN1CCN(c2ccc(-c3cnc4[nH]cc(-c5cc6n(c5)CCNC6=O)c4c3)cn2)CC1] | 1329 |
| 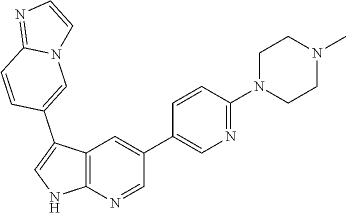 [SMILES: CN1CCN(c2ccc(-c3cnc4[nH]cc(-c5ccc6nccn6c5)c4c3)cn2)CC1] | 1330 |
| 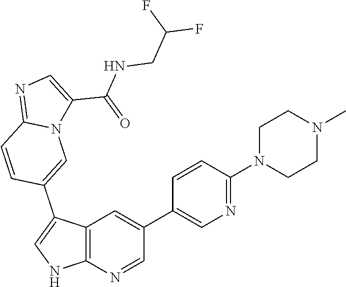 [SMILES: CN1CCN(c2ccc(-c3cnc4[nH]cc(-c5ccc6ncc(C(=O)NCC(F)F)n6c5)c4c3)cn2)CC1] | 1331 |
| 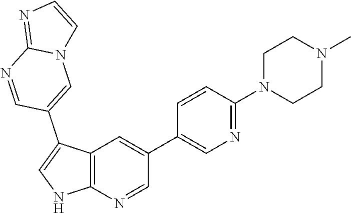 [SMILES: CN1CCN(c2ccc(-c3cnc4[nH]cc(-c5cnc6nccn6c5)c4c3)cn2)CC1] | 1332 |
| 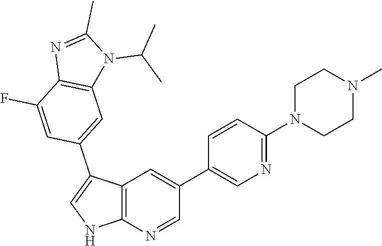 [SMILES: Cc1nc2c(F)cc(-c3c[nH]c4ncc(-c5ccc(N6CCN(C)CC6)nc5)cc34)cc2n1C(C)C] | 1333 |
| 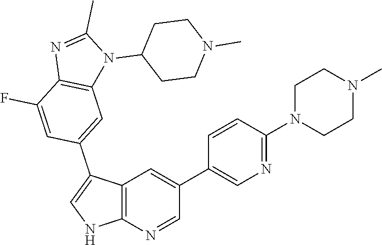 [SMILES: Cc1nc2c(F)cc(-c3c[nH]c4ncc(-c5ccc(N6CCN(C)CC6)nc5)cc34)cc2n1C1CCN(C)CC1] | 1334 |
| 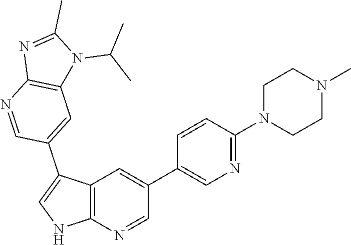 [SMILES: Cc1nc2ncc(-c3c[nH]c4ncc(-c5ccc(N6CCN(C)CC6)nc5)cc34)cc2n1C(C)C] | 1335 |
| 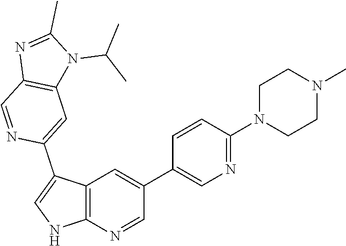 [SMILES: Cc1nc2cnc(-c3c[nH]c4ncc(-c5ccc(N6CCN(C)CC6)nc5)cc34)cc2n1C(C)C] | 1336 |
| 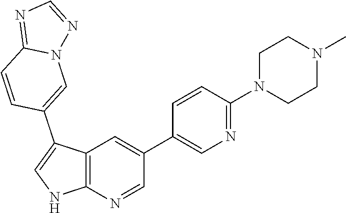 [SMILES: CN1CCN(c2ccc(-c3cnc4[nH]cc(-c5ccc6ncnn6c5)c4c3)cn2)CC1] | 1337 |
| 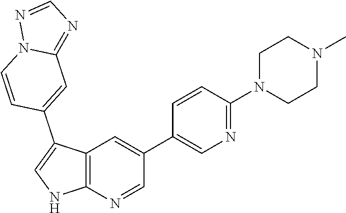 [SMILES: CN1CCN(c2ccc(-c3cnc4[nH]cc(-c5ccn6ncnc6c5)c4c3)cn2)CC1] | 1338 |
| 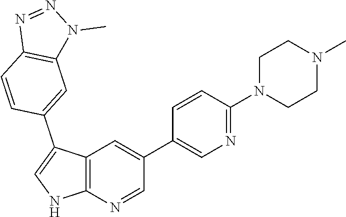 [SMILES: CN1CCN(c2ccc(-c3cnc4[nH]cc(-c5ccc6nnn(C)c6c5)c4c3)cn2)CC1] | 1339 |
| 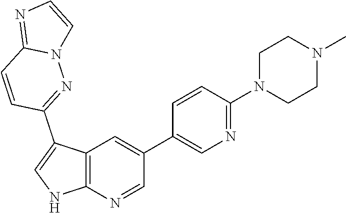 [SMILES: CN1CCN(c2ccc(-c3cnc4[nH]cc(-c5ccc6nccn6n5)c4c3)cn2)CC1] | 1340 |
| 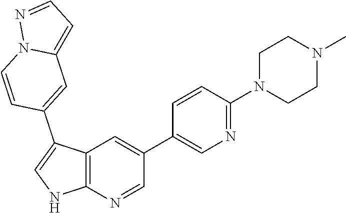 [SMILES: CN1CCN(c2ccc(-c3cnc4[nH]cc(-c5ccn6nccc6c5)c4c3)cn2)CC1] | 1341 |
| 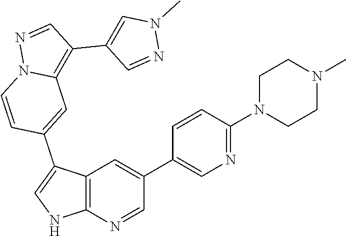 [SMILES: CN1CCN(c2ccc(-c3cnc4[nH]cc(-c5ccn6ncc(-c7cnn(C)c7)c6c5)c4c3)cn2)CC1] | 1342 |
| 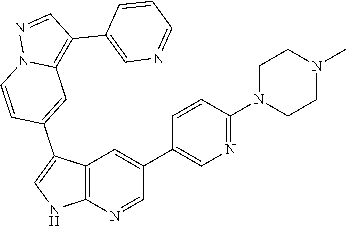 [SMILES: CN1CCN(c2ccc(-c3cnc4[nH]cc(-c5ccn6ncc(-c7cccnc7)c6c5)c4c3)cn2)CC1] | 1343 |
| 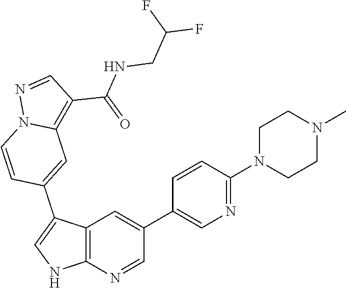 [SMILES: CN1CCN(c2ccc(-c3cnc4[nH]cc(-c5ccn6ncc(C(=O)NCC(F)F)c6c5)c4c3)cn2)CC1] | 1344 |
| 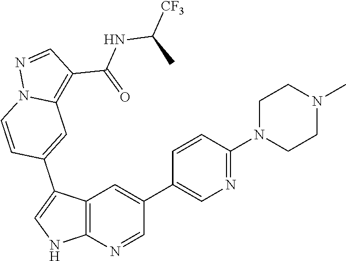 [SMILES: C[C@@H](NC(=O)c1cnn2ccc(-c3c[nH]c4ncc(-c5ccc(N6CCN(C)CC6)nc5)cc34)cc12)C(F)(F)F] | 1345 |
| 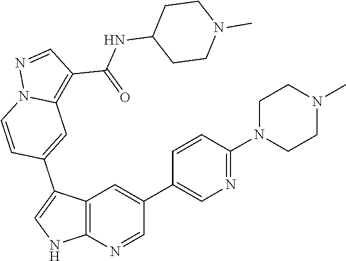 [SMILES: CN1CCC(NC(=O)c2cnn3ccc(-c4c[nH]c5ncc(-c6ccc(N7CCN(C)CC7)nc6)cc45)cc23)CC1] | 1346 |
| 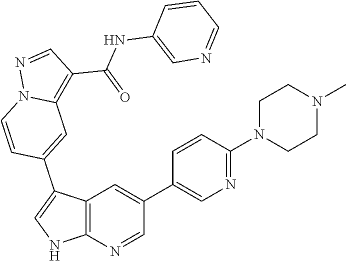 [SMILES: CN1CCN(c2ccc(-c3cnc4[nH]cc(-c5ccn6ncc(C(=O)Nc7cccnc7)c6c5)c4c3)cn2)CC1] | 1347 |
| 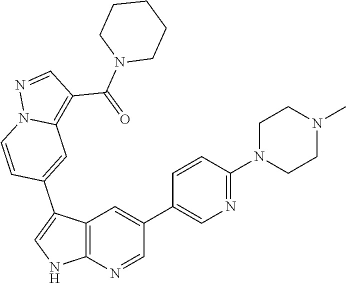 [SMILES: CN1CCN(c2ccc(-c3cnc4[nH]cc(-c5ccn6ncc(C(=O)N7CCCCC7)c6c5)c4c3)cn2)CC1] | 1348 |
| 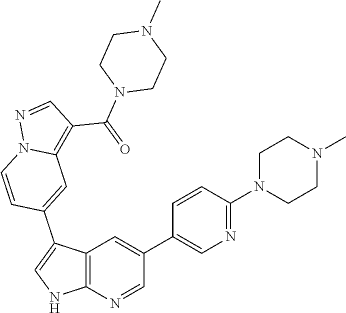 [SMILES: CN1CCN(C(=O)c2cnn3ccc(-c4c[nH]c5ncc(-c6ccc(N7CCN(C)CC7)nc6)cc45)cc23)CC1] | 1349 |
| 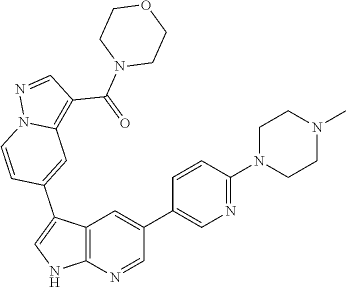 [SMILES: CN1CCN(c2ccc(-c3cnc4[nH]cc(-c5ccn6ncc(C(=O)N7CCOCC7)c6c5)c4c3)cn2)CC1] | 1350 |
| 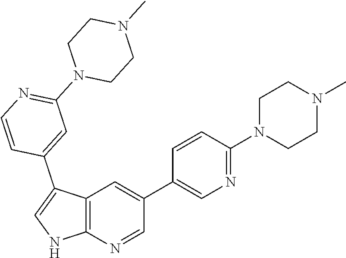 [SMILES: CN1CCN(c2ccc(-c3cnc4[nH]cc(-c5ccnc(N6CCN(C)CC6)c5)c4c3)cn2)CC1] | 1351 |
| 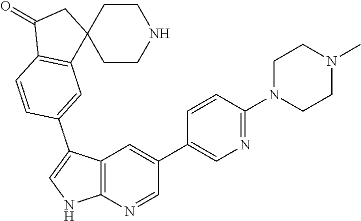 [SMILES: CN1CCN(c2ccc(-c3cnc4[nH]cc(-c5ccc6c(c5)C5(CCNCC5)CC6=O)c4c3)cn2)CC1] | 1352 |
| 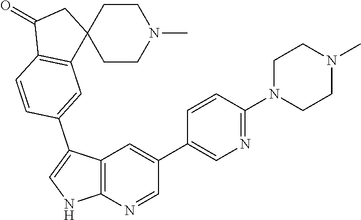 [SMILES: CN1CCN(c2ccc(-c3cnc4[nH]cc(-c5ccc6c(c5)C5(CCN(C)CC5)CC6=O)c4c3)cn2)CC1] | 1353 |
| 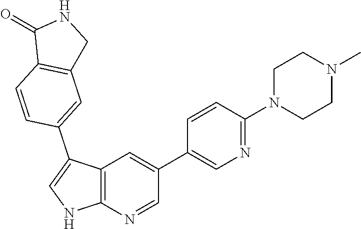 [SMILES: CN1CCN(c2ccc(-c3cnc4[nH]cc(-c5ccc6c(c5)CNC6=O)c4c3)cn2)CC1] | 1354 |
| 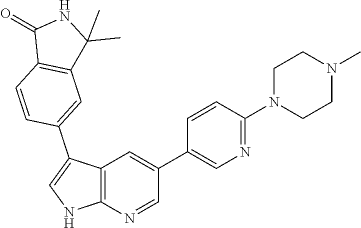 [SMILES: CN1CCN(c2ccc(-c3cnc4[nH]cc(-c5ccc6c(c5)C(C)(C)NC6=O)c4c3)cn2)CC1] | 1355 |
| 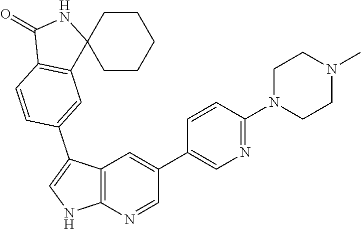 [SMILES: CN1CCN(c2ccc(-c3cnc4[nH]cc(-c5ccc6c(c5)C5(CCCCC5)NC6=O)c4c3)cn2)CC1] | 1356 |
| 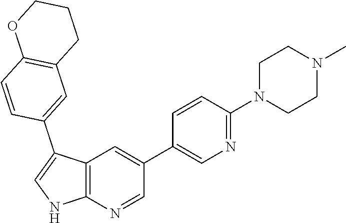 [SMILES: CN1CCN(c2ccc(-c3cnc4[nH]cc(-c5ccc6c(c5)CCCO6)c4c3)cn2)CC1] | 1357 |
| 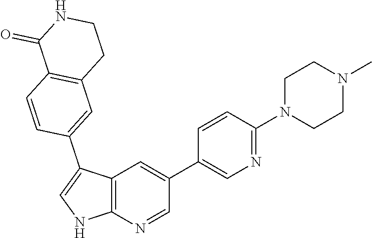 [SMILES: CN1CCN(c2ccc(-c3cnc4[nH]cc(-c5ccc6c(c5)CCNC6=O)c4c3)cn2)CC1] | 1358 |
| 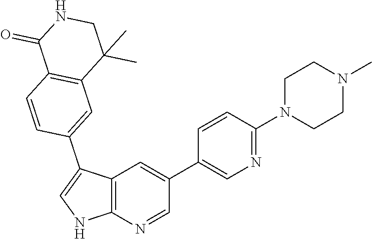 [SMILES: CN1CCN(c2ccc(-c3cnc4[nH]cc(-c5ccc6c(c5)C(C)(C)CNC6=O)c4c3)cn2)CC1] | 1359 |
| 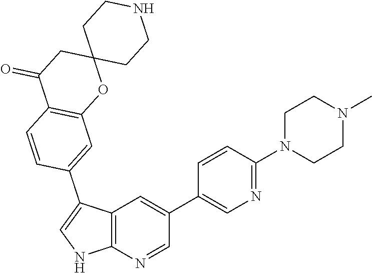 [SMILES: CN1CCN(c2ccc(-c3cnc4[nH]cc(-c5ccc6c(c5)OC5(CCNCC5)CC6=O)c4c3)cn2)CC1] | 1360 |
| 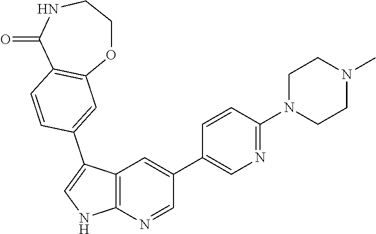 [SMILES: CN1CCN(c2ccc(-c3cnc4[nH]cc(-c5ccc6c(c5)OCCNC6=O)c4c3)cn2)CC1] | 1361 |
| 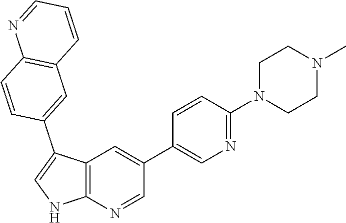 [SMILES: CN1CCN(c2ccc(-c3cnc4[nH]cc(-c5ccc6ncccc6c5)c4c3)cn2)CC1] | 1362 |
| 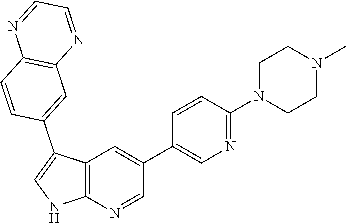 [SMILES: CN1CCN(c2ccc(-c3cnc4[nH]cc(-c5ccc6nccnc6c5)c4c3)cn2)CC1] | 1363 |
| 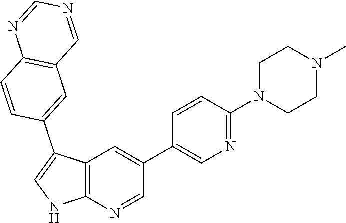 [SMILES: CN1CCN(c2ccc(-c3cnc4[nH]cc(-c5ccc6ncncc6c5)c4c3)cn2)CC1] | 1364 |
| 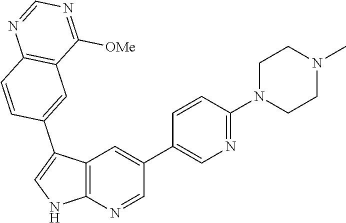 [SMILES: COc1ncnc2ccc(-c3c[nH]c4ncc(-c5ccc(N6CCN(C)CC6)nc5)cc34)cc12] | 1365 |
| 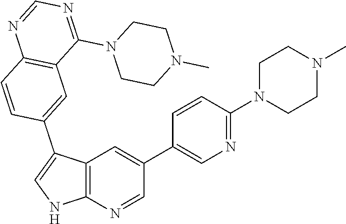 [SMILES: CN1CCN(c2ccc(-c3cnc4[nH]cc(-c5ccc6ncnc(N7CCN(C)CC7)c6c5)c4c3)cn2)CC1] | 1366 |
| 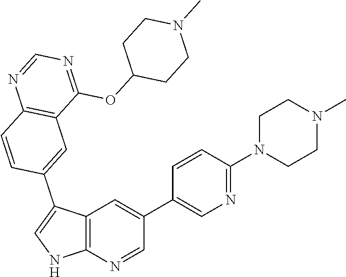 [SMILES: CN1CCC(Oc2ncnc3ccc(-c4c[nH]c5ncc(-c6ccc(N7CCN(C)CC7)nc6)cc45)cc23)CC1] | 1367 |
| 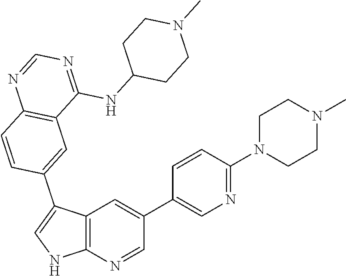 [SMILES: CN1CCC(Nc2ncnc3ccc(-c4c[nH]c5ncc(-c6ccc(N7CCN(C)CC7)nc6)cc45)cc23)CC1] | 1368 |
| 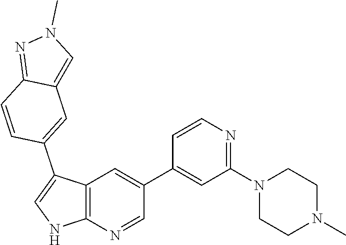 [SMILES: CN1CCN(c2cc(-c3cnc4[nH]cc(-c5ccc6nn(C)cc6c5)c4c3)ccn2)CC1] | 1369 |
| 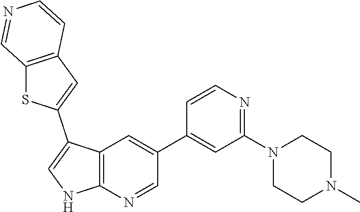 [SMILES: CN1CCN(c2cc(-c3cnc4[nH]cc(-c5cc6ccncc6s5)c4c3)ccn2)CC1] | 1370 |
| 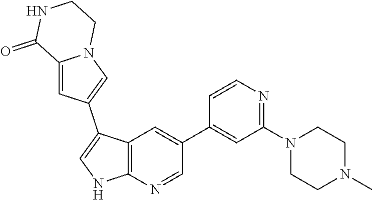 [SMILES: CN1CCN(c2cc(-c3cnc4[nH]cc(-c5cc6n(c5)CCNC6=O)c4c3)ccn2)CC1] | 1371 |
| 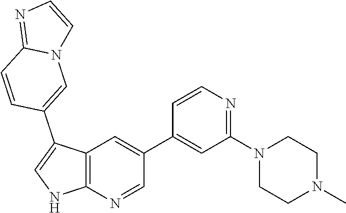 [SMILES: CN1CCN(c2cc(-c3cnc4[nH]cc(-c5ccc6nccn6c5)c4c3)ccn2)CC1] | 1372 |
| 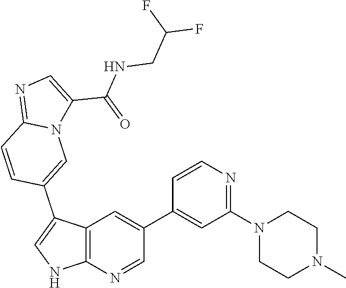 [SMILES: CN1CCN(c2cc(-c3cnc4[nH]cc(-c5ccc6ncc(C(=O)NCC(F)F)n6c5)c4c3)ccn2)CC1] | 1373 |
| 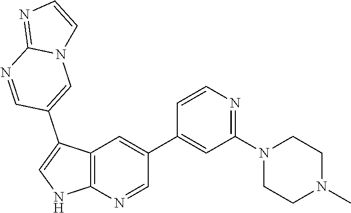 [SMILES: CN1CCN(c2cc(-c3cnc4[nH]cc(-c5cnc6nccn6c5)c4c3)ccn2)CC1] | 1374 |
| 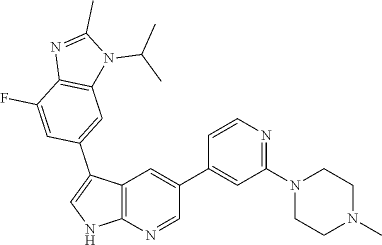 [SMILES: Cc1nc2c(F)cc(-c3c[nH]c4ncc(-c5ccnc(N6CCN(C)CC6)c5)cc34)cc2n1C(C)C] | 1375 |
| 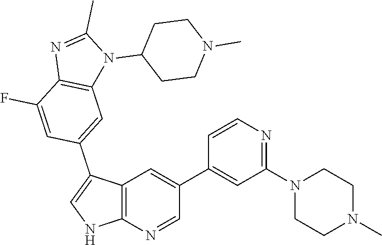 [SMILES: Cc1nc2c(F)cc(-c3c[nH]c4ncc(-c5ccnc(N6CCN(C)CC6)c5)cc34)cc2n1C1CCN(C)CC1] | 1376 |
| 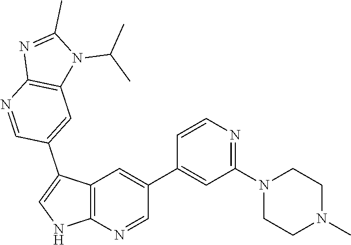 [SMILES: Cc1nc2ncc(-c3c[nH]c4ncc(-c5ccnc(N6CCN(C)CC6)c5)cc34)cc2n1C(C)C] | 1377 |
| 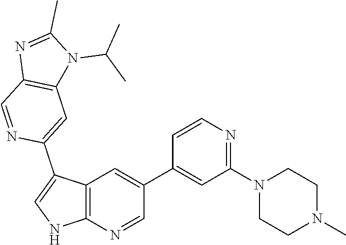 [SMILES: Cc1nc2cnc(-c3c[nH]c4ncc(-c5ccnc(N6CCN(C)CC6)c5)cc34)cc2n1C(C)C] | 1378 |
| 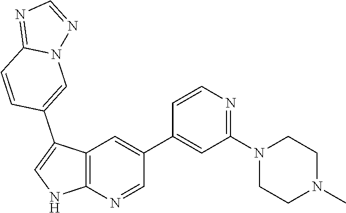 [SMILES: CN1CCN(c2cc(-c3cnc4[nH]cc(-c5ccc6ncnn6c5)c4c3)ccn2)CC1] | 1379 |
| 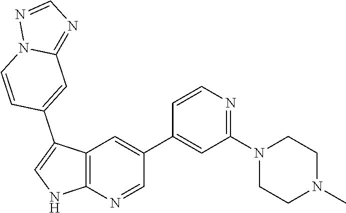 [SMILES: CN1CCN(c2cc(-c3cnc4[nH]cc(-c5ccn6ncnc6c5)c4c3)ccn2)CC1] | 1380 |
| 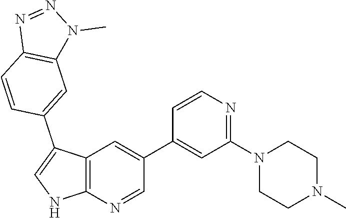 [SMILES: CN1CCN(c2cc(-c3cnc4[nH]cc(-c5ccc6nnn(C)c6c5)c4c3)ccn2)CC1] | 1381 |
| 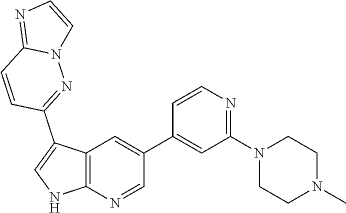 [SMILES: CN1CCN(c2cc(-c3cnc4[nH]cc(-c5ccc6nccn6n5)c4c3)ccn2)CC1] | 1382 |
| 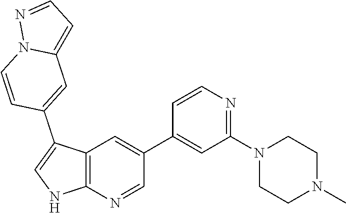 [SMILES: CN1CCN(c2cc(-c3cnc4[nH]cc(-c5ccn6nccc6c5)c4c3)ccn2)CC1] | 1383 |
| 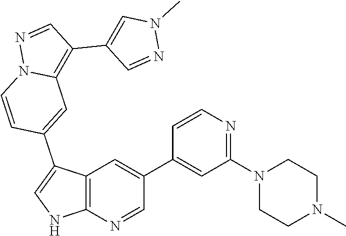 [SMILES: CN1CCN(c2cc(-c3cnc4[nH]cc(-c5ccn6ncc(-c7cnn(C)c7)c6c5)c4c3)ccn2)CC1] | 1384 |
| 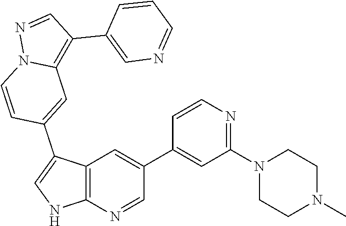 [SMILES: CN1CCN(c2cc(-c3cnc4[nH]cc(-c5ccn6ncc(-c7cccnc7)c6c5)c4c3)ccn2)CC1] | 1385 |
| 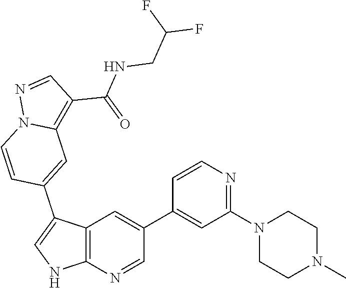 [SMILES: CN1CCN(c2cc(-c3cnc4[nH]cc(-c5ccn6ncc(C(=O)NCC(F)F)c6c5)c4c3)ccn2)CC1] | 1386 |
| 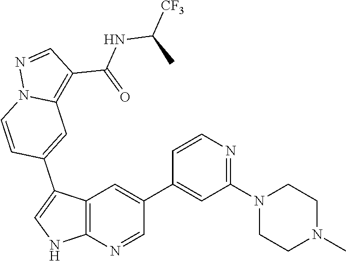 [SMILES: C[C@@H](NC(=O)c1cnn2ccc(-c3c[nH]c4ncc(-c5ccnc(N6CCN(C)CC6)c5)cc34)cc12)C(F)(F)F] | 1387 |
| 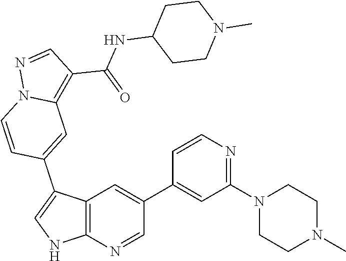 [SMILES: CN1CCC(NC(=O)c2cnn3ccc(-c4c[nH]c5ncc(-c6ccnc(N7CCN(C)CC7)c6)cc45)cc23)CC1] | 1388 |
| 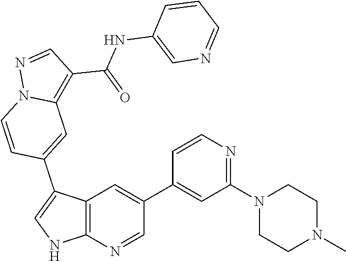 [SMILES: CN1CCN(c2cc(-c3cnc4[nH]cc(-c5ccn6ncc(C(=O)Nc7cccnc7)c6c5)c4c3)ccn2)CC1] | 1389 |
| 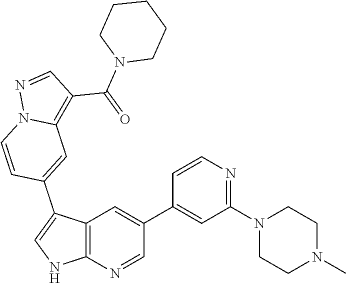 [SMILES: CN1CCN(c2cc(-c3cnc4[nH]cc(-c5ccn6ncc(C(=O)N7CCCCC7)c6c5)c4c3)ccn2)CC1] | 1390 |
| 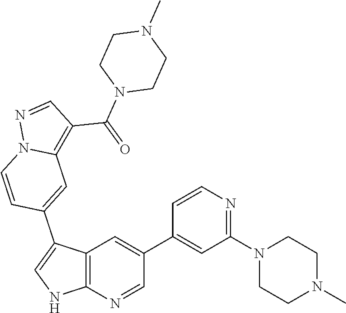 [SMILES: CN1CCN(C(=O)c2cnn3ccc(-c4c[nH]c5ncc(-c6ccnc(N7CCN(C)CC7)c6)cc45)cc23)CC1] | 1391 |
| 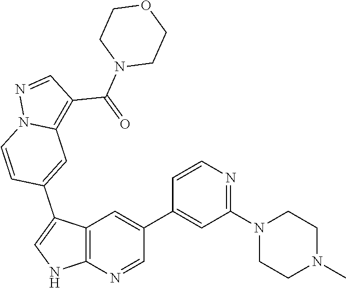 [SMILES: CN1CCN(c2cc(-c3cnc4[nH]cc(-c5ccn6ncc(C(=O)N7CCOCC7)c6c5)c4c3)ccn2)CC1] | 1392 |
| 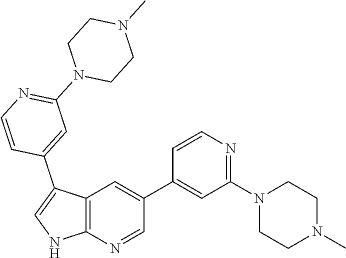 [SMILES: CN1CCN(c2cc(-c3cnc4[nH]cc(-c5ccnc(N6CCN(C)CC6)c5)c4c3)ccn2)CC1] | 1393 |
| 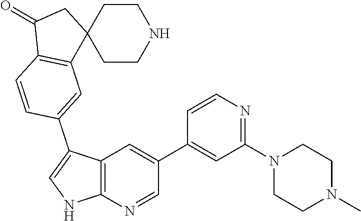 [SMILES: CN1CCN(c2cc(-c3cnc4[nH]cc(-c5ccc6c(c5)C5(CCNCC5)CC6=O)c4c3)ccn2)CC1] | 1394 |
| 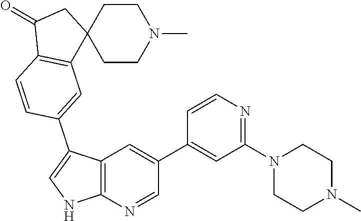 [SMILES: CN1CCN(c2cc(-c3cnc4[nH]cc(-c5ccc6c(c5)C5(CCN(C)CC5)CC6=O)c4c3)ccn2)CC1] | 1395 |
| 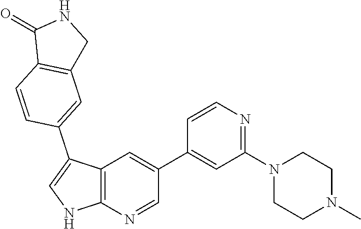 [SMILES: CN1CCN(c2cc(-c3cnc4[nH]cc(-c5ccc6c(c5)CNC6=O)c4c3)ccn2)CC1] | 1396 |
| 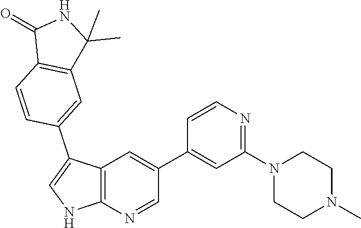 [SMILES: CN1CCN(c2cc(-c3cnc4[nH]cc(-c5ccc6c(c5)C(C)(C)NC6=O)c4c3)ccn2)CC1] | 1397 |
| 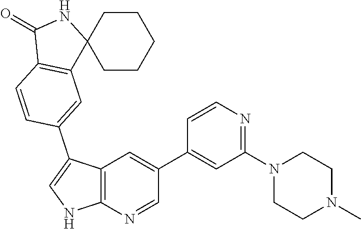 [SMILES: CN1CCN(c2cc(-c3cnc4[nH]cc(-c5ccc6c(c5)C5(CCCCC5)NC6=O)c4c3)ccn2)CC1] | 1398 |
| 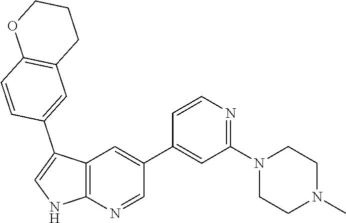 [SMILES: CN1CCN(c2cc(-c3cnc4[nH]cc(-c5ccc6c(c5)CCCO6)c4c3)ccn2)CC1] | 1399 |
| 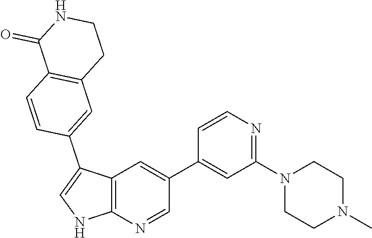 [SMILES: CN1CCN(c2cc(-c3cnc4[nH]cc(-c5ccc6c(c5)CCNC6=O)c4c3)ccn2)CC1] | 1400 |
| 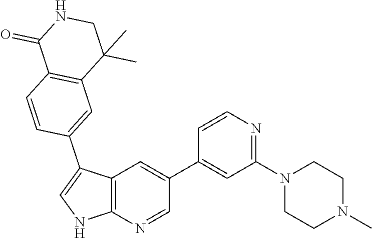 [SMILES: CN1CCN(c2cc(-c3cnc4[nH]cc(-c5ccc6c(c5)C(C)(C)CNC6=O)c4c3)ccn2)CC1] | 1401 |
| 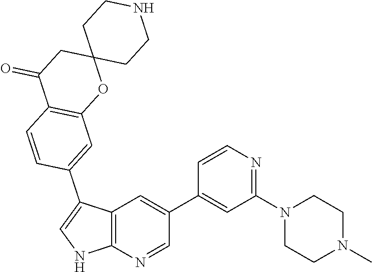 [SMILES: CN1CCN(c2cc(-c3cnc4[nH]cc(-c5ccc6c(c5)OC5(CCNCC5)CC6=O)c4c3)ccn2)CC1] | 1402 |
| 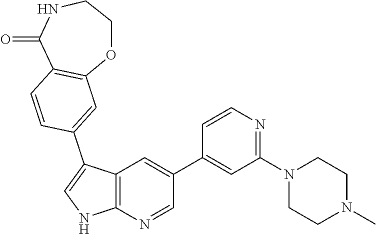 [SMILES: CN1CCN(c2cc(-c3cnc4[nH]cc(-c5ccc6c(c5)OCCNC6=O)c4c3)ccn2)CC1] | 1403 |
| 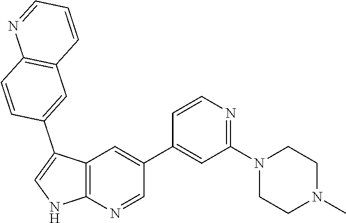 [SMILES: CN1CCN(c2cc(-c3cnc4[nH]cc(-c5ccc6ncccc6c5)c4c3)ccn2)CC1] | 1404 |
| 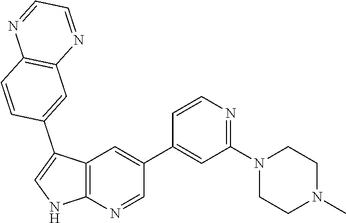 [SMILES: CN1CCN(c2cc(-c3cnc4[nH]cc(-c5ccc6nccnc6c5)c4c3)ccn2)CC1] | 1405 |
| 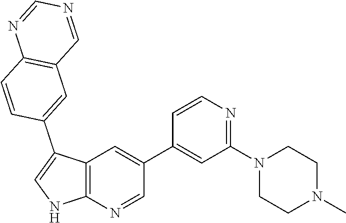 [SMILES: CN1CCN(c2cc(-c3cnc4[nH]cc(-c5ccc6ncncc6c5)c4c3)ccn2)CC1] | 1406 |
| 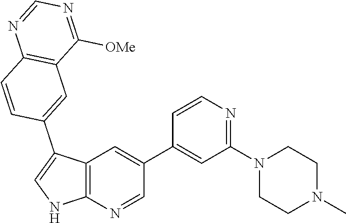 [SMILES: COc1ncnc2ccc(-c3c[nH]c4ncc(-c5ccnc(N6CCN(C)CC6)c5)cc34)cc12] | 1407 |
| 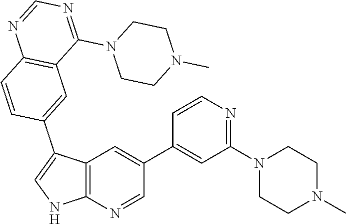 [SMILES: CN1CCN(c2cc(-c3cnc4[nH]cc(-c5ccc6ncnc(N7CCN(C)CC7)c6c5)c4c3)ccn2)CC1] | 1408 |
| 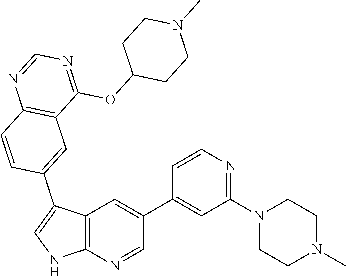 [SMILES: CN1CCC(Oc2ncnc3ccc(-c4c[nH]c5ncc(-c6ccnc(N7CCN(C)CC7)c6)cc45)cc23)CC1] | 1409 |
| 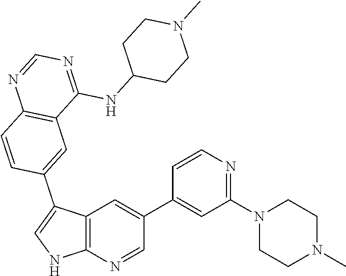 [SMILES: CN1CCC(Nc2ncnc3ccc(-c4c[nH]c5ncc(-c6ccnc(N7CCN(C)CC7)c6)cc45)cc23)CC1] | 1410 |
| 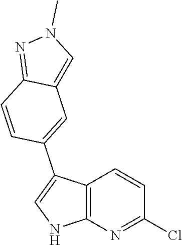 [SMILES: Cn1cc2cc(-c3c[nH]c4nc(Cl)ccc34)ccc2n1] | 1411 |
| 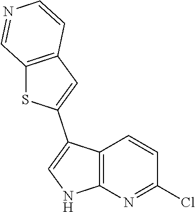 [SMILES: Clc1ccc2c(-c3cc4ccncc4s3)c[nH]c2n1] | 1412 |
| 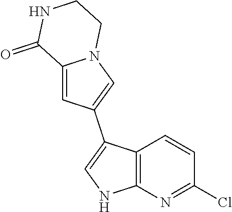 [SMILES: O=C1NCCn2cc(-c3c[nH]c4nc(Cl)ccc34)cc21] | 1413 |
| 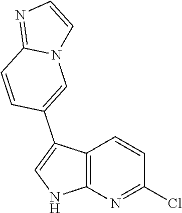 [SMILES: Clc1ccc2c(-c3ccc4nccn4c3)c[nH]c2n1] | 1414 |
| 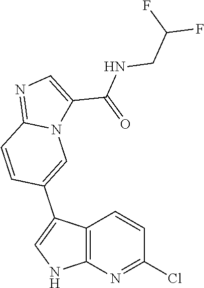 [SMILES: O=C(NCC(F)F)c1cnc2ccc(-c3c[nH]c4nc(Cl)ccc34)cn12] | 1415 |
| 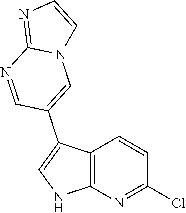 [SMILES: Clc1ccc2c(-c3cnc4nccn4c3)c[nH]c2n1] | 1416 |
| 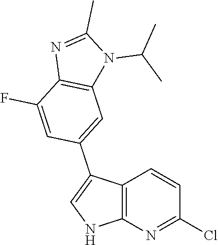 [SMILES: Cc1nc2c(F)cc(-c3c[nH]c4nc(Cl)ccc34)cc2n1C(C)C] | 1417 |
| 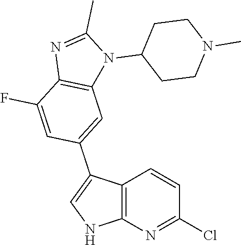 [SMILES: Cc1nc2c(F)cc(-c3c[nH]c4nc(Cl)ccc34)cc2n1C1CCN(C)CC1] | 1418 |
| 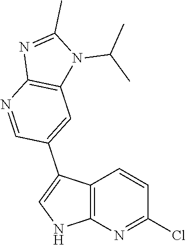 [SMILES: Cc1nc2ncc(-c3c[nH]c4nc(Cl)ccc34)cc2n1C(C)C] | 1419 |
| 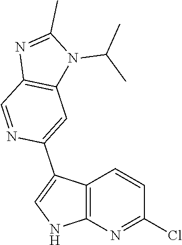 [SMILES: Cc1nc2cnc(-c3c[nH]c4nc(Cl)ccc34)cc2n1C(C)C] | 1420 |
| 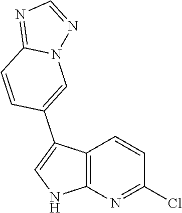 [SMILES: Clc1ccc2c(-c3ccc4ncnn4c3)c[nH]c2n1] | 1421 |
| 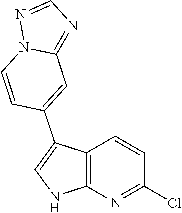 [SMILES: Clc1ccc2c(-c3ccn4ncnc4c3)c[nH]c2n1] | 1422 |
| 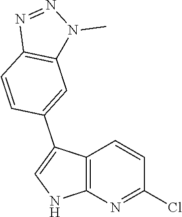 [SMILES: Cn1nnc2ccc(-c3c[nH]c4nc(Cl)ccc34)cc21] | 1423 |
| 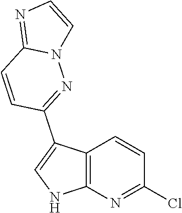 [SMILES: Clc1ccc2c(-c3ccc4nccn4n3)c[nH]c2n1] | 1424 |
| 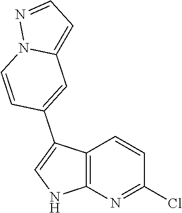 [SMILES: Clc1ccc2c(-c3ccn4nccc4c3)c[nH]c2n1] | 1425 |
| 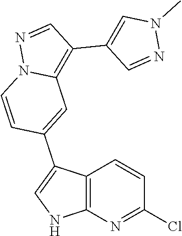 [SMILES: Cn1cc(-c2cnn3ccc(-c4c[nH]c5nc(Cl)ccc45)cc23)cn1] | 1426 |
| 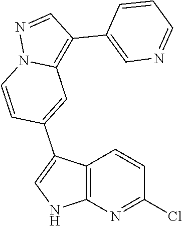 [SMILES: Clc1ccc2c(-c3ccn4ncc(-c5cccnc5)c4c3)c[nH]c2n1] | 1427 |
| 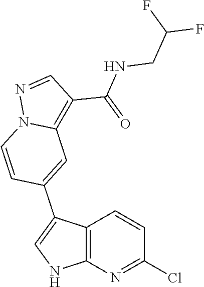 [SMILES: O=C(NCC(F)F)c1cnn2ccc(-c3c[nH]c4nc(Cl)ccc34)cc12] | 1428 |
| 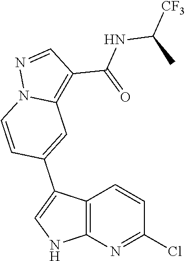 [SMILES: C[C@@H](NC(=O)c1cnn2ccc(-c3c[nH]c4nc(Cl)ccc34)cc12)C(F)(F)F] | 1429 |
| 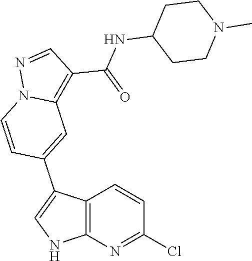 [SMILES: CN1CCC(NC(=O)c2cnn3ccc(-c4c[nH]c5nc(Cl)ccc45)cc23)CC1] | 1430 |
| 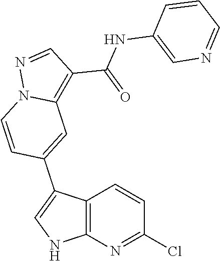 [SMILES: O=C(Nc1cccnc1)c1cnn2ccc(-c3c[nH]c4nc(Cl)ccc34)cc12] | 1431 |
| 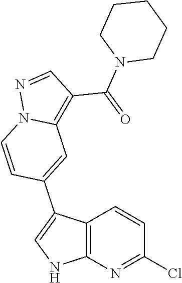 [SMILES: O=C(c1cnn2ccc(-c3c[nH]c4nc(Cl)ccc34)cc12)N1CCCCC1] | 1432 |
| 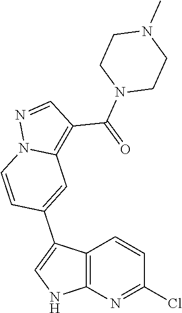 [SMILES: CN1CCN(C(=O)c2cnn3ccc(-c4c[nH]c5nc(Cl)ccc45)cc23)CC1] | 1433 |
| 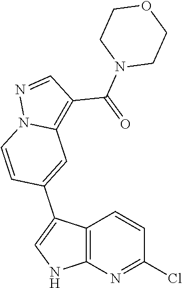 [SMILES: O=C(c1cnn2ccc(-c3c[nH]c4nc(Cl)ccc34)cc12)N1CCOCC1] | 1434 |
| 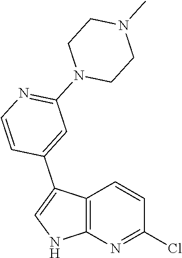 [SMILES: CN1CCN(c2cc(-c3c[nH]c4nc(Cl)ccc34)ccn2)CC1] | 1435 |
| 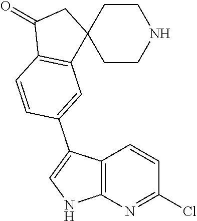 [SMILES: O=C1CC2(CCNCC2)c2cc(-c3c[nH]c4nc(Cl)ccc34)ccc21] | 1436 |
| 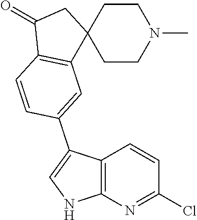 [SMILES: CN1CCC2(CC1)CC(=O)c1ccc(-c3c[nH]c4nc(Cl)ccc34)cc12] | 1437 |
| 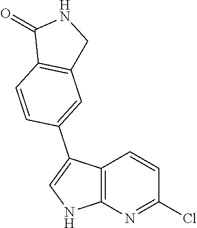 [SMILES: O=C1NCc2cc(-c3c[nH]c4nc(Cl)ccc34)ccc21] | 1438 |
| 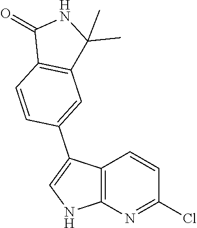 [SMILES: CC1(C)NC(=O)c2ccc(-c3c[nH]c4nc(Cl)ccc34)cc21] | 1439 |
| 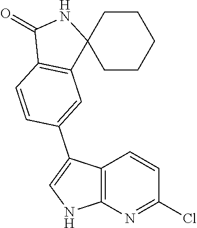 [SMILES: O=C1NC2(CCCCC2)c2cc(-c3c[nH]c4nc(Cl)ccc34)ccc21] | 1440 |
| 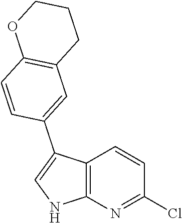 [SMILES: Clc1ccc2c(-c3ccc4c(c3)CCCO4)c[nH]c2n1] | 1441 |
| 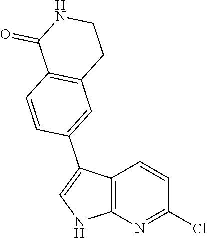 [SMILES: O=C1NCCc2cc(-c3c[nH]c4nc(Cl)ccc34)ccc21] | 1442 |
| 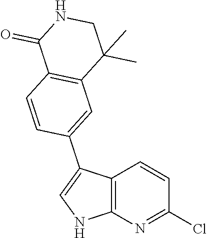 [SMILES: CC1(C)CNC(=O)c2ccc(-c3c[nH]c4nc(Cl)ccc34)cc21] | 1443 |
| 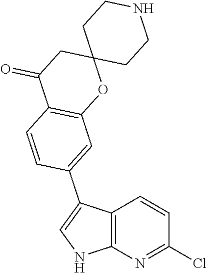 [SMILES: O=C1CC2(CCNCC2)Oc2cc(-c3c[nH]c4nc(Cl)ccc34)ccc21] | 1444 |
| 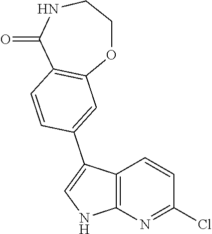 [SMILES: O=C1NCCOc2cc(-c3c[nH]c4nc(Cl)ccc34)ccc21] | 1445 |
| 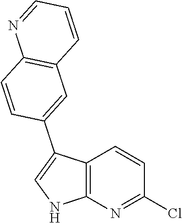 [SMILES: Clc1ccc2c(-c3ccc4ncccc4c3)c[nH]c2n1] | 1446 |
| 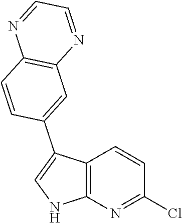 [SMILES: Clc1ccc2c(-c3ccc4nccnc4c3)c[nH]c2n1] | 1447 |
| 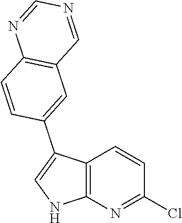 [SMILES: Clc1ccc2c(-c3ccc4ncncc4c3)c[nH]c2n1] | 1448 |
| 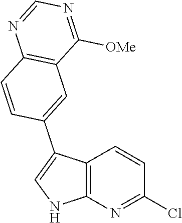 [SMILES: COc1ncnc2ccc(-c3c[nH]c4nc(Cl)ccc34)cc12] | 1449 |
| 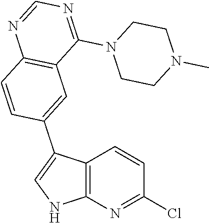 [SMILES: CN1CCN(c2ncnc3ccc(-c4c[nH]c5nc(Cl)ccc45)cc23)CC1] | 1450 |
| 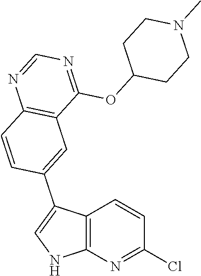 [SMILES: CN1CCC(Oc2ncnc3ccc(-c4c[nH]c5nc(Cl)ccc45)cc23)CC1] | 1451 |
| 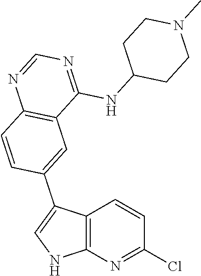 [SMILES: CN1CCC(Nc2ncnc3ccc(-c4c[nH]c5nc(Cl)ccc45)cc23)CC1] | 1452 |
| 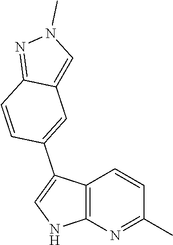 [SMILES: Cc1ccc2c(-c3ccc4nn(C)cc4c3)c[nH]c2n1] | 1453 |
| 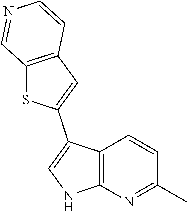 [SMILES: Cc1ccc2c(-c3cc4ccncc4s3)c[nH]c2n1] | 1454 |
| 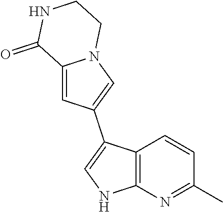 [SMILES: Cc1ccc2c(-c3cc4n(c3)CCNC4=O)c[nH]c2n1] | 1455 |
| 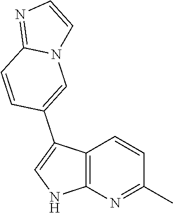 [SMILES: Cc1ccc2c(-c3ccc4nccn4c3)c[nH]c2n1] | 1456 |
| 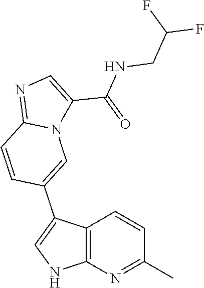 [SMILES: Cc1ccc2c(-c3ccc4ncc(C(=O)NCC(F)F)n4c3)c[nH]c2n1] | 1457 |
| 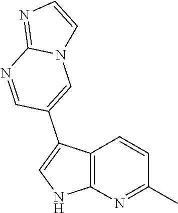 [SMILES: Cc1ccc2c(-c3cnc4nccn4c3)c[nH]c2n1] | 1458 |
| 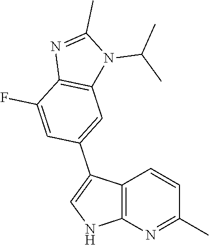 [SMILES: Cc1ccc2c(-c3cc(F)c4nc(C)n(C(C)C)c4c3)c[nH]c2n1] | 1459 |
| 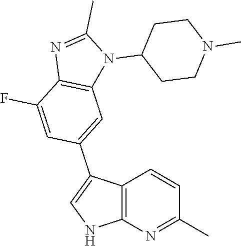 [SMILES: Cc1ccc2c(-c3cc(F)c4nc(C)n(C5CCN(C)CC5)c4c3)c[nH]c2n1] | 1460 |
| 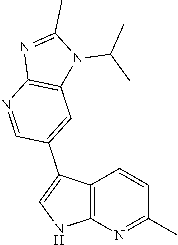 [SMILES: Cc1ccc2c(-c3cnc4nc(C)n(C(C)C)c4c3)c[nH]c2n1] | 1461 |
| 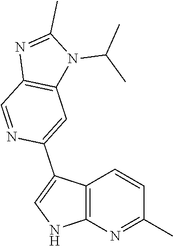 [SMILES: Cc1ccc2c(-c3cc4c(cn3)nc(C)n4C(C)C)c[nH]c2n1] | 1462 |
| 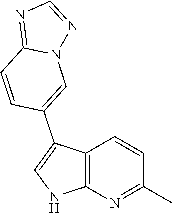 [SMILES: Cc1ccc2c(-c3ccc4ncnn4c3)c[nH]c2n1] | 1463 |
| 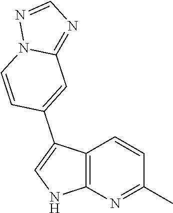 [SMILES: Cc1ccc2c(-c3ccn4ncnc4c3)c[nH]c2n1] | 1464 |
| 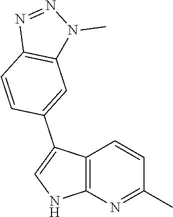 [SMILES: Cc1ccc2c(-c3ccc4nnn(C)c4c3)c[nH]c2n1] | 1465 |
| 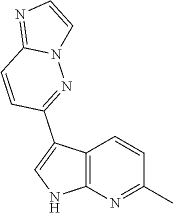 [SMILES: Cc1ccc2c(-c3ccc4nccn4n3)c[nH]c2n1] | 1466 |
| 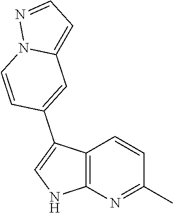 [SMILES: Cc1ccc2c(-c3ccn4nccc4c3)c[nH]c2n1] | 1467 |
| 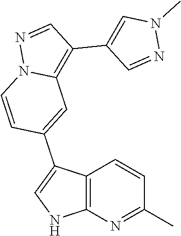 [SMILES: Cc1ccc2c(-c3ccn4ncc(-c5cnn(C)c5)c4c3)c[nH]c2n1] | 1468 |
| 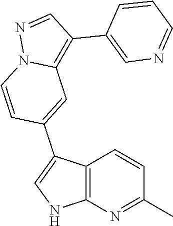 [SMILES: Cc1ccc2c(-c3ccn4ncc(-c5cccnc5)c4c3)c[nH]c2n1] | 1469 |
| 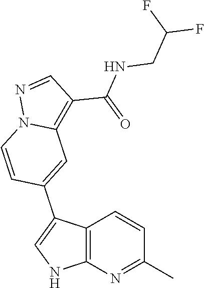 [SMILES: Cc1ccc2c(-c3ccn4ncc(C(=O)NCC(F)F)c4c3)c[nH]c2n1] | 1470 |
| 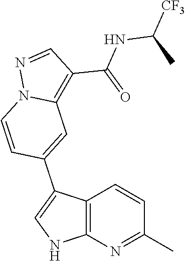 [SMILES: Cc1ccc2c(-c3ccn4ncc(C(=O)N[C@H](C)C(F)(F)F)c4c3)c[nH]c2n1] | 1471 |
| 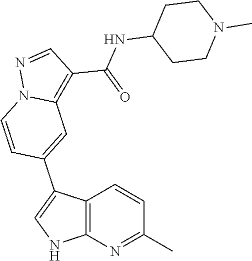 [SMILES: Cc1ccc2c(-c3ccn4ncc(C(=O)NC5CCN(C)CC5)c4c3)c[nH]c2n1] | 1472 |
| 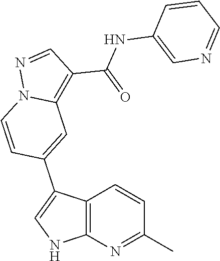 [SMILES: Cc1ccc2c(-c3ccn4ncc(C(=O)Nc5cccnc5)c4c3)c[nH]c2n1] | 1473 |
| 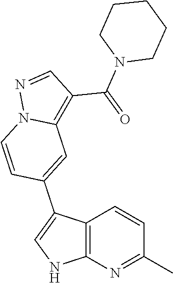 [SMILES: Cc1ccc2c(-c3ccn4ncc(C(=O)N5CCCCC5)c4c3)c[nH]c2n1] | 1474 |
| 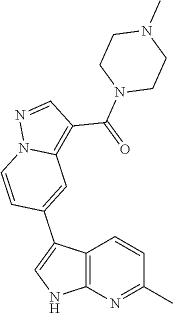 [SMILES: Cc1ccc2c(-c3ccn4ncc(C(=O)N5CCN(C)CC5)c4c3)c[nH]c2n1] | 1475 |
| 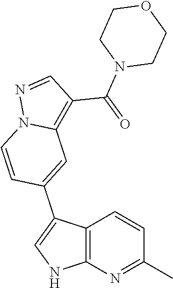 [SMILES: Cc1ccc2c(-c3ccn4ncc(C(=O)N5CCOCC5)c4c3)c[nH]c2n1] | 1476 |
| 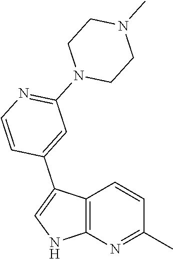 [SMILES: Cc1ccc2c(-c3ccnc(N4CCN(C)CC4)c3)c[nH]c2n1] | 1477 |
| 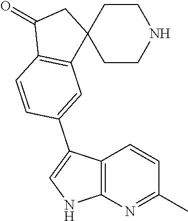 [SMILES: Cc1ccc2c(-c3ccc4c(c3)C3(CCNCC3)CC4=O)c[nH]c2n1] | 1478 |
| 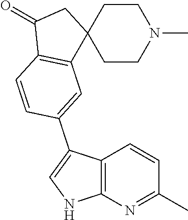 [SMILES: Cc1ccc2c(-c3ccc4c(c3)C3(CCN(C)CC3)CC4=O)c[nH]c2n1] | 1479 |
| 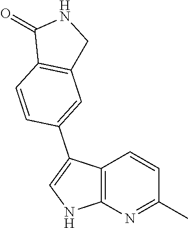 [SMILES: Cc1ccc2c(-c3ccc4c(c3)CNC4=O)c[nH]c2n1] | 1480 |
| 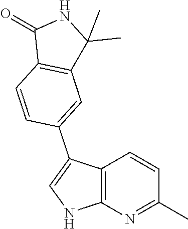 [SMILES: Cc1ccc2c(-c3ccc4c(c3)C(C)(C)NC4=O)c[nH]c2n1] | 1481 |
| 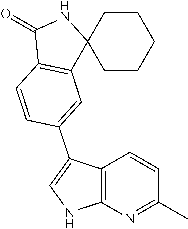 [SMILES: Cc1ccc2c(-c3ccc4c(c3)C3(CCCCC3)NC4=O)c[nH]c2n1] | 1482 |
| 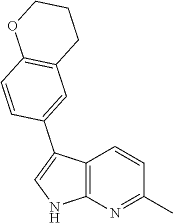 [SMILES: Cc1ccc2c(-c3ccc4c(c3)CCCO4)c[nH]c2n1] | 1483 |
| 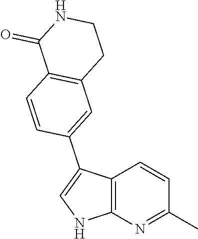 [SMILES: Cc1ccc2c(-c3ccc4c(c3)CCNC4=O)c[nH]c2n1] | 1484 |
| 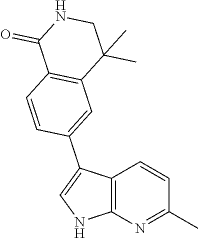 [SMILES: Cc1ccc2c(-c3ccc4c(c3)C(C)(C)CNC4=O)c[nH]c2n1] | 1485 |
| 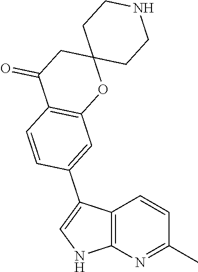 [SMILES: Cc1ccc2c(-c3ccc4c(c3)OC3(CCNCC3)CC4=O)c[nH]c2n1] | 1486 |
| 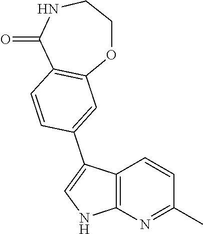 [SMILES: Cc1ccc2c(-c3ccc4c(c3)OCCNC4=O)c[nH]c2n1] | 1487 |
| 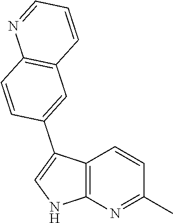 [SMILES: Cc1ccc2c(-c3ccc4ncccc4c3)c[nH]c2n1] | 1488 |
| 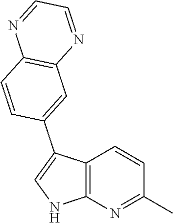 [SMILES: Cc1ccc2c(-c3ccc4nccnc4c3)c[nH]c2n1] | 1489 |
| 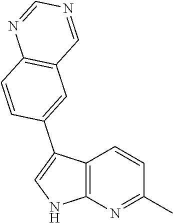 [SMILES: Cc1ccc2c(-c3ccc4ncncc4c3)c[nH]c2n1] | 1490 |
| 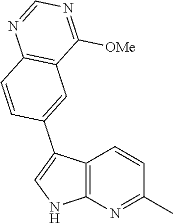 [SMILES: COc1ncnc2ccc(-c3c[nH]c4nc(C)ccc34)cc12] | 1491 |
| 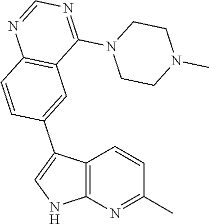 [SMILES: Cc1ccc2c(-c3ccc4ncnc(N5CCN(C)CC5)c4c3)c[nH]c2n1] | 1492 |
| 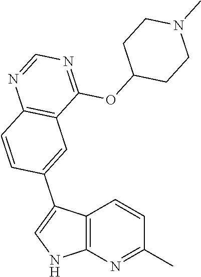 [SMILES: Cc1ccc2c(-c3ccc4ncnc(OC5CCN(C)CC5)c4c3)c[nH]c2n1] | 1493 |
| 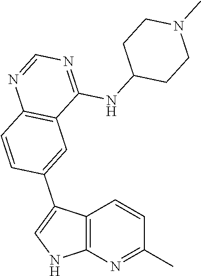 [SMILES: Cc1ccc2c(-c3ccc4ncnc(NC5CCN(C)CC5)c4c3)c[nH]c2n1] | 1494 |
| 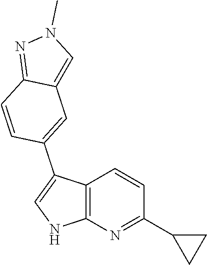 [SMILES: Cn1cc2cc(-c3c[nH]c4nc(C5CC5)ccc34)ccc2n1] | 1495 |
| 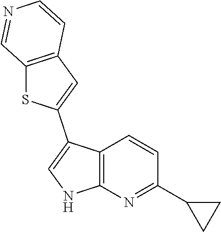 [SMILES: c1cc2cc(-c3c[nH]c4nc(C5CC5)ccc34)sc2cn1] | 1496 |
| 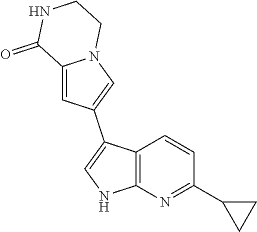 [SMILES: O=C1NCCn2cc(-c3c[nH]c4nc(C5CC5)ccc34)cc21] | 1497 |
| 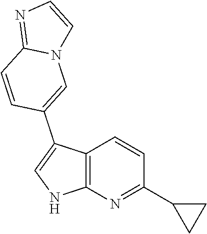 [SMILES: c1cn2cc(-c3c[nH]c4nc(C5CC5)ccc34)ccc2n1] | 1498 |
| 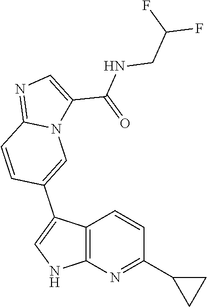 [SMILES: O=C(NCC(F)F)c1cnc2ccc(-c3c[nH]c4nc(C5CC5)ccc34)cn12] | 1499 |
| 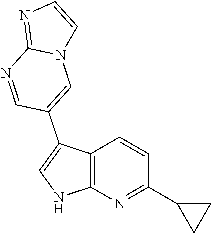 [SMILES: c1cn2cc(-c3c[nH]c4nc(C5CC5)ccc34)cnc2n1] | 1500 |
| 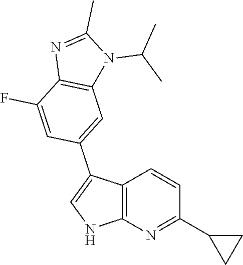 [SMILES: Cc1nc2c(F)cc(-c3c[nH]c4nc(C5CC5)ccc34)cc2n1C(C)C] | 1501 |
| 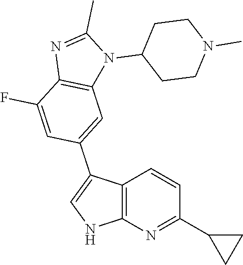 [SMILES: Cc1nc2c(F)cc(-c3c[nH]c4nc(C5CC5)ccc34)cc2n1C1CCN(C)CC1] | 1502 |
| 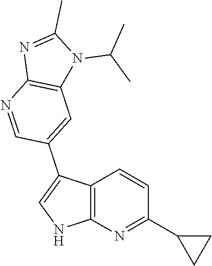 [SMILES: Cc1nc2ncc(-c3c[nH]c4nc(C5CC5)ccc34)cc2n1C(C)C] | 1503 |
| 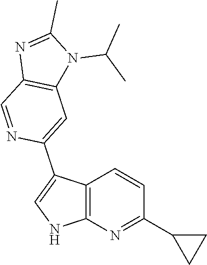 [SMILES: Cc1nc2cnc(-c3c[nH]c4nc(C5CC5)ccc34)cc2n1C(C)C] | 1504 |
| 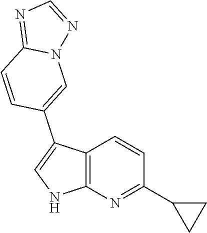 [SMILES: c1nc2ccc(-c3c[nH]c4nc(C5CC5)ccc34)cn2n1] | 1505 |
| 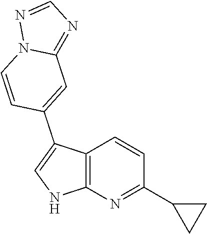 [SMILES: c1nc2cc(-c3c[nH]c4nc(C5CC5)ccc34)ccn2n1] | 1506 |
| 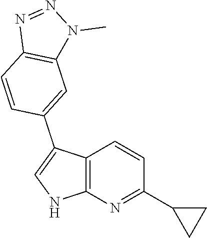 [SMILES: Cn1nnc2ccc(-c3c[nH]c4nc(C5CC5)ccc34)cc21] | 1507 |
| 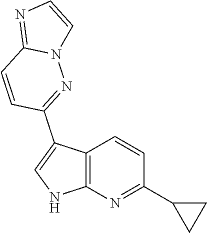 [SMILES: c1cn2nc(-c3c[nH]c4nc(C5CC5)ccc34)ccc2n1] | 1508 |
| 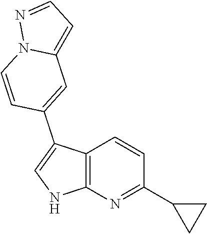 [SMILES: c1cc2cc(-c3c[nH]c4nc(C5CC5)ccc34)ccn2n1] | 1509 |
| 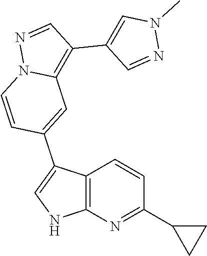 [SMILES: Cn1cc(-c2cnn3ccc(-c4c[nH]c5nc(C6CC6)ccc45)cc23)cn1] | 1510 |
| 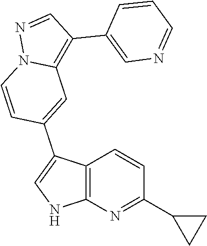 [SMILES: c1cncc(-c2cnn3ccc(-c4c[nH]c5nc(C6CC6)ccc45)cc23)c1] | 1511 |
| 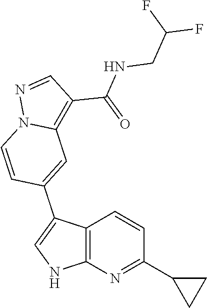 [SMILES: O=C(NCC(F)F)c1cnn2ccc(-c3c[nH]c4nc(C5CC5)ccc34)cc12] | 1512 |
| 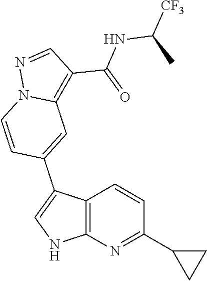 [SMILES: C[C@@H](NC(=O)c1cnn2ccc(-c3c[nH]c4nc(C5CC5)ccc34)cc12)C(F)(F)F] | 1513 |
| 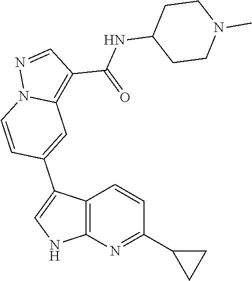 [SMILES: CN1CCC(NC(=O)c2cnn3ccc(-c4c[nH]c5nc(C6CC6)ccc45)cc23)CC1] | 1514 |
| 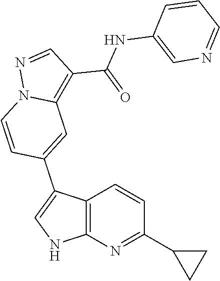 [SMILES: O=C(Nc1cccnc1)c1cnn2ccc(-c3c[nH]c4nc(C5CC5)ccc34)cc12] | 1515 |
| 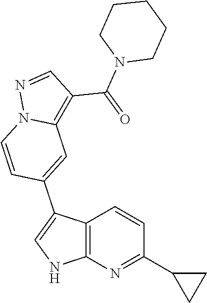 [SMILES: O=C(c1cnn2ccc(-c3c[nH]c4nc(C5CC5)ccc34)cc12)N1CCCCC1] | 1516 |
| 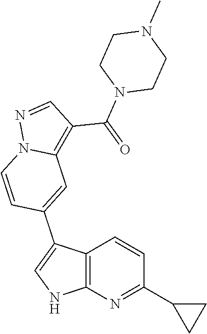 [SMILES: CN1CCN(C(=O)c2cnn3ccc(-c4c[nH]c5nc(C6CC6)ccc45)cc23)CC1] | 1517 |
| 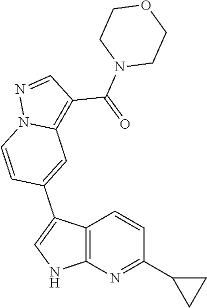 [SMILES: O=C(c1cnn2ccc(-c3c[nH]c4nc(C5CC5)ccc34)cc12)N1CCOCC1] | 1518 |
| 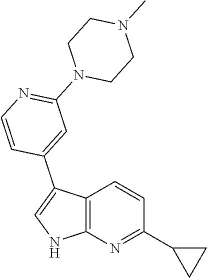 [SMILES: CN1CCN(c2cc(-c3c[nH]c4nc(C5CC5)ccc34)ccn2)CC1] | 1519 |
| 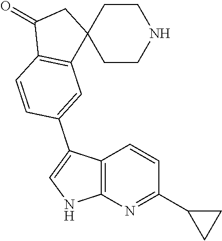 [SMILES: O=C1CC2(CCNCC2)c2cc(-c3c[nH]c4nc(C5CC5)ccc34)ccc21] | 1520 |
| 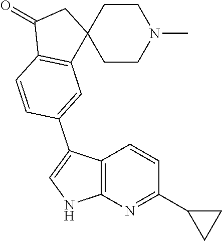 [SMILES: CN1CCC2(CC1)CC(=O)c1ccc(-c3c[nH]c4nc(C5CC5)ccc34)cc12] | 1521 |
| 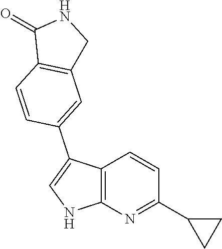 [SMILES: O=C1NCc2cc(-c3c[nH]c4nc(C5CC5)ccc34)ccc21] | 1522 |
| 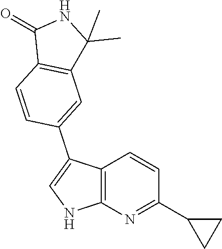 [SMILES: CC1(C)NC(=O)c2ccc(-c3c[nH]c4nc(C5CC5)ccc34)cc21] | 1523 |
| 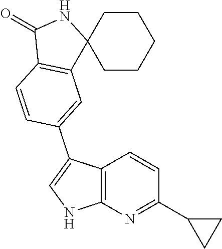 [SMILES: O=C1NC2(CCCCC2)c2cc(-c3c[nH]c4nc(C5CC5)ccc34)ccc21] | 1524 |
| 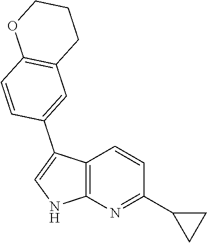 [SMILES: c1cc2c(cc1-c1c[nH]c3nc(C4CC4)ccc13)CCCO2] | 1525 |
| 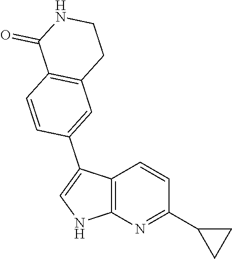 [SMILES: O=C1NCCc2cc(-c3c[nH]c4nc(C5CC5)ccc34)ccc21] | 1526 |
| 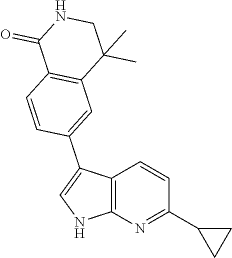 [SMILES: CC1(C)CNC(=O)c2ccc(-c3c[nH]c4nc(C5CC5)ccc34)cc21] | 1527 |
| 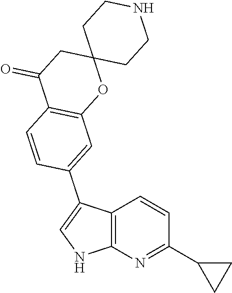 [SMILES: O=C1CC2(CCNCC2)Oc2cc(-c3c[nH]c4nc(C5CC5)ccc34)ccc21] | 1528 |
| 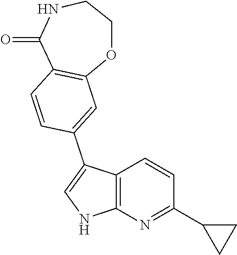 [SMILES: O=C1NCCOc2cc(-c3c[nH]c4nc(C5CC5)ccc34)ccc21] | 1529 |
| 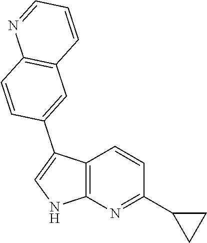 [SMILES: c1cnc2ccc(-c3c[nH]c4nc(C5CC5)ccc34)cc2c1] | 1530 |
| 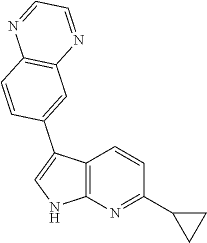 [SMILES: c1cnc2cc(-c3c[nH]c4nc(C5CC5)ccc34)ccc2n1] | 1531 |
| 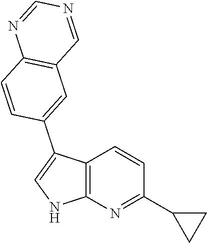 [SMILES: c1ncc2cc(-c3c[nH]c4nc(C5CC5)ccc34)ccc2n1] | 1532 |
| 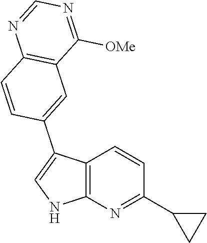 [SMILES: COc1ncnc2ccc(-c3c[nH]c4nc(C5CC5)ccc34)cc12] | 1533 |
| 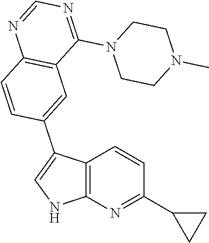 [SMILES: CN1CCN(c2ncnc3ccc(-c4c[nH]c5nc(C6CC6)ccc45)cc23)CC1] | 1534 |
| 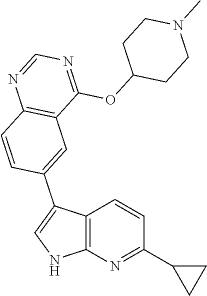 [SMILES: CN1CCC(Oc2ncnc3ccc(-c4c[nH]c5nc(C6CC6)ccc45)cc23)CC1] | 1535 |
| 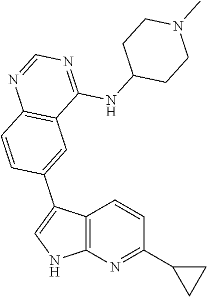 [SMILES: CN1CCC(Nc2ncnc3ccc(-c4c[nH]c5nc(C6CC6)ccc45)cc23)CC1] | 1536 |
| 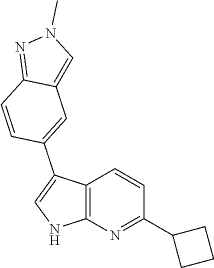 [SMILES: Cn1cc2cc(-c3c[nH]c4nc(C5CCC5)ccc34)ccc2n1] | 1537 |
| 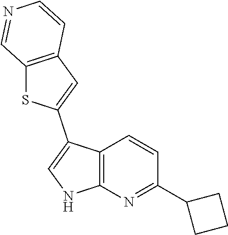 [SMILES: c1cc2cc(-c3c[nH]c4nc(C5CCC5)ccc34)sc2cn1] | 1538 |
| 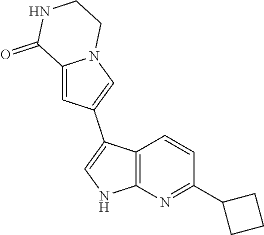 [SMILES: O=C1NCCn2cc(-c3c[nH]c4nc(C5CCC5)ccc34)cc21] | 1539 |
| 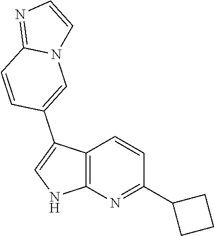 [SMILES: c1cn2cc(-c3c[nH]c4nc(C5CCC5)ccc34)ccc2n1] | 1540 |
| 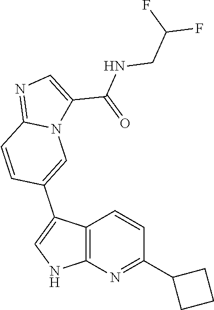 [SMILES: O=C(NCC(F)F)c1cnc2ccc(-c3c[nH]c4nc(C5CCC5)ccc34)cn12] | 1541 |
| 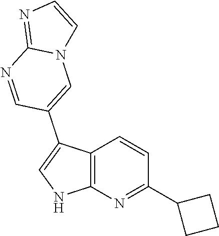 [SMILES: c1cn2cc(-c3c[nH]c4nc(C5CCC5)ccc34)cnc2n1] | 1542 |
| 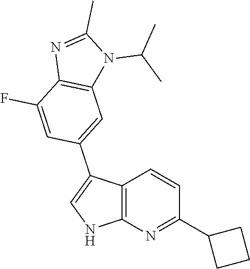 [SMILES: Cc1nc2c(F)cc(-c3c[nH]c4nc(C5CCC5)ccc34)cc2n1C(C)C] | 1543 |
| 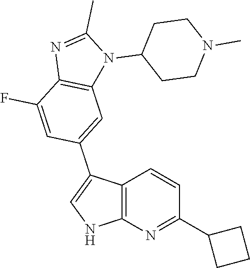 [SMILES: Cc1nc2c(F)cc(-c3c[nH]c4nc(C5CCC5)ccc34)cc2n1C1CCN(C)CC1] | 1544 |
| 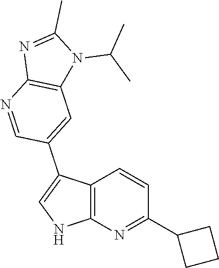 [SMILES: Cc1nc2ncc(-c3c[nH]c4nc(C5CCC5)ccc34)cc2n1C(C)C] | 1545 |
| 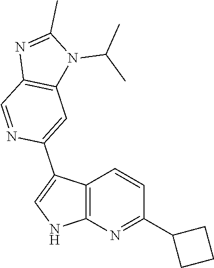 [SMILES: Cc1nc2cnc(-c3c[nH]c4nc(C5CCC5)ccc34)cc2n1C(C)C] | 1546 |
| 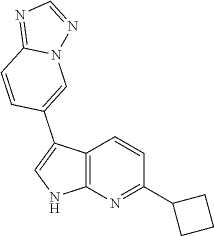 [SMILES: c1nc2ccc(-c3c[nH]c4nc(C5CCC5)ccc34)cn2n1] | 1547 |
| 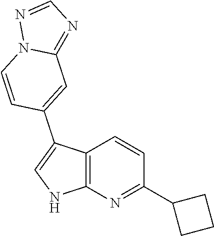 [SMILES: c1nc2cc(-c3c[nH]c4nc(C5CCC5)ccc34)ccn2n1] | 1548 |
| 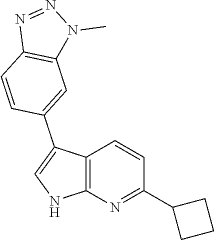 [SMILES: Cn1nnc2ccc(-c3c[nH]c4nc(C5CCC5)ccc34)cc21] | 1549 |
| 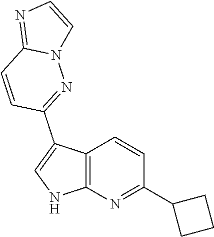 [SMILES: c1cn2nc(-c3c[nH]c4nc(C5CCC5)ccc34)ccc2n1] | 1550 |
| 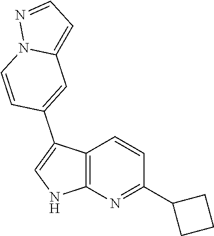 [SMILES: c1cc2cc(-c3c[nH]c4nc(C5CCC5)ccc34)ccn2n1] | 1551 |
| 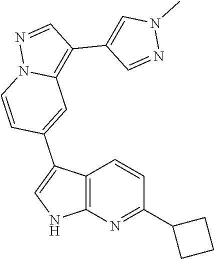 [SMILES: Cn1cc(-c2cnn3ccc(-c4c[nH]c5nc(C6CCC6)ccc45)cc23)cn1] | 1552 |
| 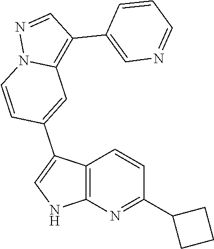 [SMILES: c1cncc(-c2cnn3ccc(-c4c[nH]c5nc(C6CCC6)ccc45)cc23)c1] | 1553 |
| 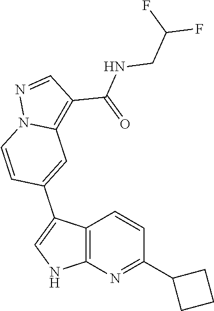 [SMILES: O=C(NCC(F)F)c1cnn2ccc(-c3c[nH]c4nc(C5CCC5)ccc34)cc12] | 1554 |
| 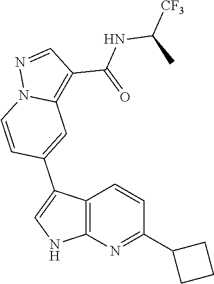 [SMILES: C[C@@H](NC(=O)c1cnn2ccc(-c3c[nH]c4nc(C5CCC5)ccc34)cc12)C(F)(F)F] | 1555 |
| 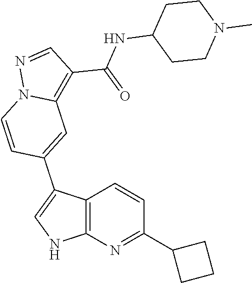 [SMILES: CN1CCC(NC(=O)c2cnn3ccc(-c4c[nH]c5nc(C6CCC6)ccc45)cc23)CC1] | 1556 |
| 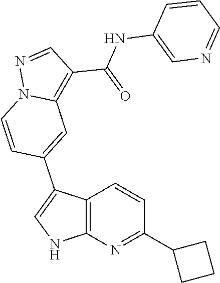 [SMILES: O=C(Nc1cccnc1)c1cnn2ccc(-c3c[nH]c4nc(C5CCC5)ccc34)cc12] | 1557 |
| 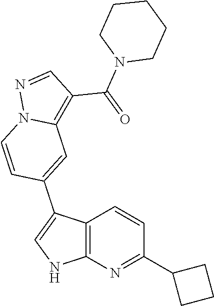 [SMILES: O=C(c1cnn2ccc(-c3c[nH]c4nc(C5CCC5)ccc34)cc12)N1CCCCC1] | 1558 |
| 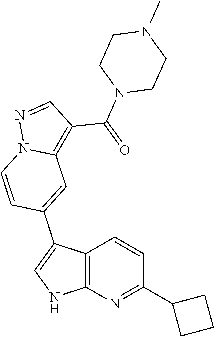 [SMILES: CN1CCN(C(=O)c2cnn3ccc(-c4c[nH]c5nc(C6CCC6)ccc45)cc23)CC1] | 1559 |
| 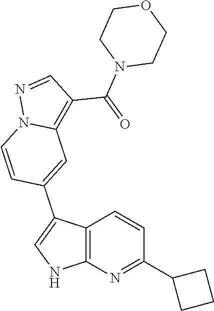 [SMILES: O=C(c1cnn2ccc(-c3c[nH]c4nc(C5CCC5)ccc34)cc12)N1CCOCC1] | 1560 |
| 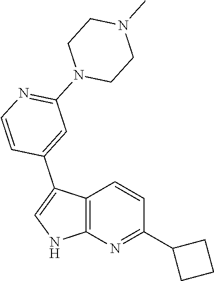 [SMILES: CN1CCN(c2cc(-c3c[nH]c4nc(C5CCC5)ccc34)ccn2)CC1] | 1561 |
| 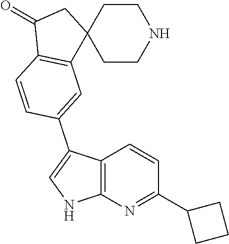 [SMILES: O=C1CC2(CCNCC2)c2cc(-c3c[nH]c4nc(C5CCC5)ccc34)ccc21] | 1562 |
| 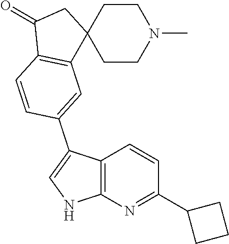 [SMILES: CN1CCC2(CC1)CC(=O)c1ccc(-c3c[nH]c4nc(C5CCC5)ccc34)cc12] | 1563 |
| 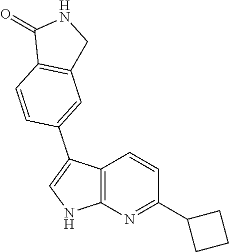 [SMILES: O=C1NCc2cc(-c3c[nH]c4nc(C5CCC5)ccc34)ccc21] | 1564 |
| 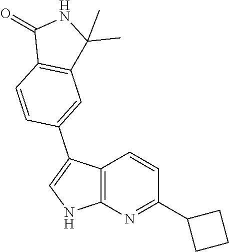 [SMILES: CC1(C)NC(=O)c2ccc(-c3c[nH]c4nc(C5CCC5)ccc34)cc21] | 1565 |
| 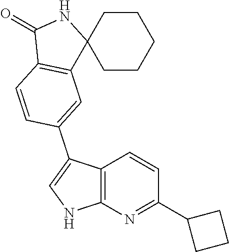 [SMILES: O=C1NC2(CCCCC2)c2cc(-c3c[nH]c4nc(C5CCC5)ccc34)ccc21] | 1566 |
| 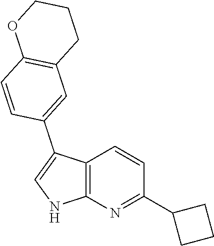 [SMILES: c1cc2c(cc1-c1c[nH]c3nc(C4CCC4)ccc13)CCCO2] | 1567 |
| 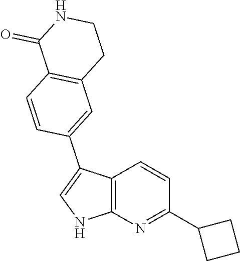 [SMILES: O=C1NCCc2cc(-c3c[nH]c4nc(C5CCC5)ccc34)ccc21] | 1568 |
| 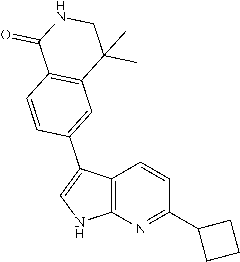 [SMILES: CC1(C)CNC(=O)c2ccc(-c3c[nH]c4nc(C5CCC5)ccc34)cc21] | 1569 |
| 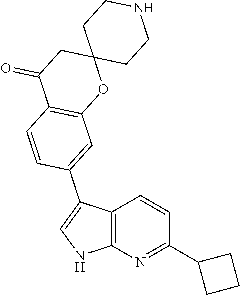 [SMILES: O=C1CC2(CCNCC2)Oc2cc(-c3c[nH]c4nc(C5CCC5)ccc34)ccc21] | 1570 |
| 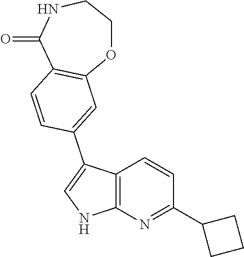 [SMILES: O=C1NCCOc2cc(-c3c[nH]c4nc(C5CCC5)ccc34)ccc21] | 1571 |
| 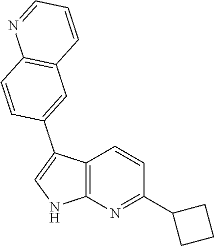 [SMILES: c1cnc2ccc(-c3c[nH]c4nc(C5CCC5)ccc34)cc2c1] | 1572 |
| 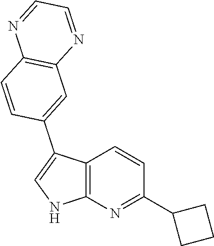 [SMILES: c1cnc2cc(-c3c[nH]c4nc(C5CCC5)ccc34)ccc2n1] | 1573 |
| 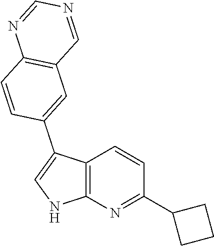 [SMILES: c1ncc2cc(-c3c[nH]c4nc(C5CCC5)ccc34)ccc2n1] | 1574 |
| 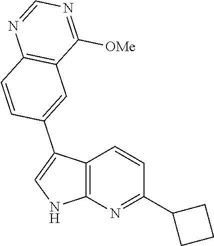 [SMILES: COc1ncnc2ccc(-c3c[nH]c4nc(C5CCC5)ccc34)cc12] | 1575 |
| 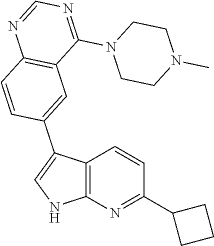 [SMILES: CN1CCN(c2ncnc3ccc(-c4c[nH]c5nc(C6CCC6)ccc45)cc23)CC1] | 1576 |
| 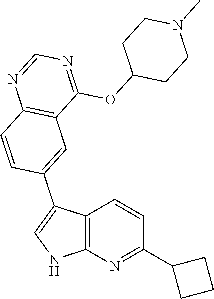 [SMILES: CN1CCC(Oc2ncnc3ccc(-c4c[nH]c5nc(C6CCC6)ccc45)cc23)CC1] | 1577 |
| 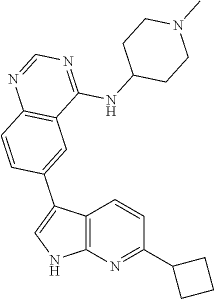 [SMILES: CN1CCC(Nc2ncnc3ccc(-c4c[nH]c5nc(C6CCC6)ccc45)cc23)CC1] | 1578 |
| 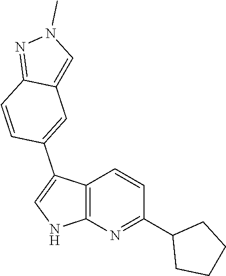 [SMILES: Cn1cc2cc(-c3c[nH]c4nc(C5CCCC5)ccc34)ccc2n1] | 1579 |
| 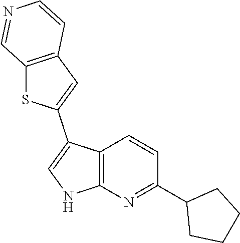 [SMILES: c1cc2cc(-c3c[nH]c4nc(C5CCCC5)ccc34)sc2cn1] | 1580 |
| 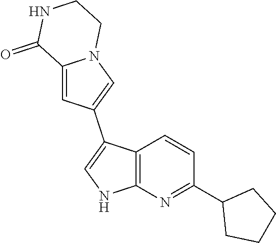 [SMILES: O=C1NCCn2cc(-c3c[nH]c4nc(C5CCCC5)ccc34)cc21] | 1581 |
| 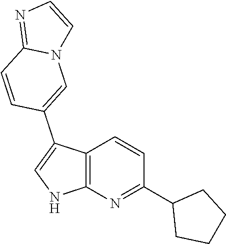 [SMILES: c1cn2cc(-c3c[nH]c4nc(C5CCCC5)ccc34)ccc2n1] | 1582 |
| 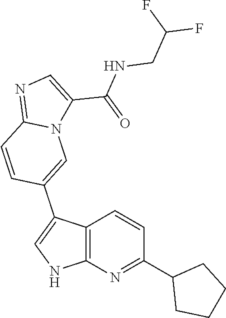 [SMILES: O=C(NCC(F)F)c1cnc2ccc(-c3c[nH]c4nc(C5CCCC5)ccc34)cn12] | 1583 |
| 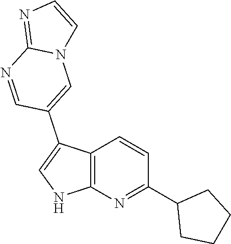 [SMILES: c1cn2cc(-c3c[nH]c4nc(C5CCCC5)ccc34)cnc2n1] | 1584 |
| 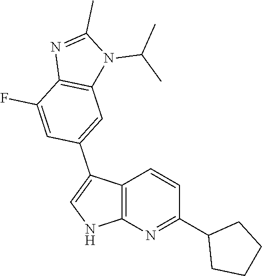 [SMILES: Cc1nc2c(F)cc(-c3c[nH]c4nc(C5CCCC5)ccc34)cc2n1C(C)C] | 1585 |
| 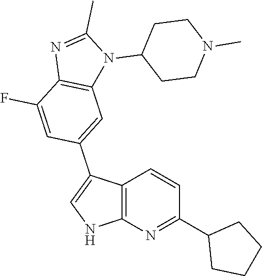 [SMILES: Cc1nc2c(F)cc(-c3c[nH]c4nc(C5CCCC5)ccc34)cc2n1C1CCN(C)CC1] | 1586 |
| 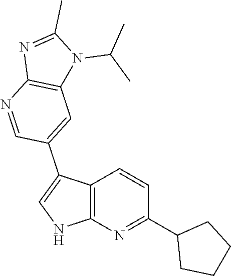 [SMILES: Cc1nc2ncc(-c3c[nH]c4nc(C5CCCC5)ccc34)cc2n1C(C)C] | 1587 |
| 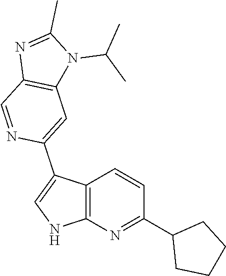 [SMILES: Cc1nc2cnc(-c3c[nH]c4nc(C5CCCC5)ccc34)cc2n1C(C)C] | 1588 |
| 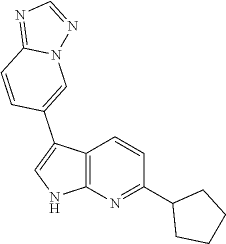 [SMILES: c1nc2ccc(-c3c[nH]c4nc(C5CCCC5)ccc34)cn2n1] | 1589 |
| 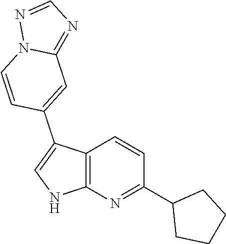 [SMILES: c1nc2cc(-c3c[nH]c4nc(C5CCCC5)ccc34)ccn2n1] | 1590 |
| 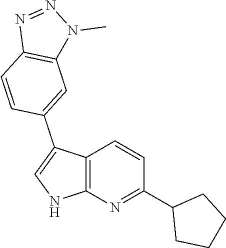 [SMILES: Cn1nnc2ccc(-c3c[nH]c4nc(C5CCCC5)ccc34)cc21] | 1591 |
| 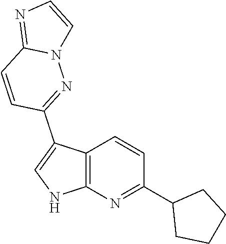 [SMILES: c1cn2nc(-c3c[nH]c4nc(C5CCCC5)ccc34)ccc2n1] | 1592 |
| 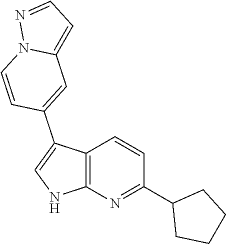 [SMILES: c1cc2cc(-c3c[nH]c4nc(C5CCCC5)ccc34)ccn2n1] | 1593 |
| 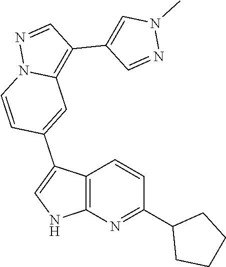 [SMILES: Cn1cc(-c2cnn3ccc(-c4c[nH]c5nc(C6CCCC6)ccc45)cc23)cn1] | 1594 |
| 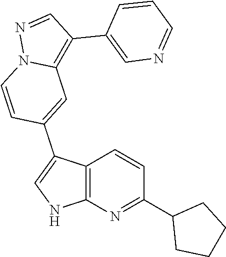 [SMILES: c1cncc(-c2cnn3ccc(-c4c[nH]c5nc(C6CCCC6)ccc45)cc23)c1] | 1595 |
| 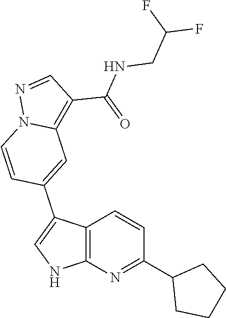 [SMILES: O=C(NCC(F)F)c1cnn2ccc(-c3c[nH]c4nc(C5CCCC5)ccc34)cc12] | 1596 |
| 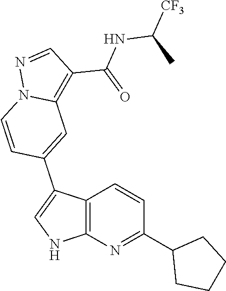 [SMILES: C[C@@H](NC(=O)c1cnn2ccc(-c3c[nH]c4nc(C5CCCC5)ccc34)cc12)C(F)(F)F] | 1597 |
| 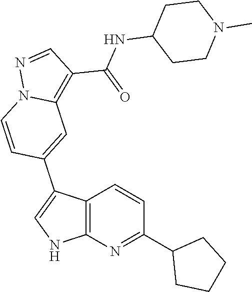 [SMILES: CN1CCC(NC(=O)c2cnn3ccc(-c4c[nH]c5nc(C6CCCC6)ccc45)cc23)CC1] | 1598 |
| 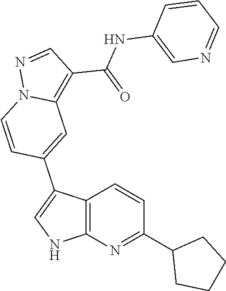 [SMILES: O=C(Nc1cccnc1)c1cnn2ccc(-c3c[nH]c4nc(C5CCCC5)ccc34)cc12] | 1599 |
| 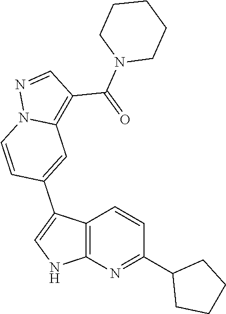 [SMILES: O=C(c1cnn2ccc(-c3c[nH]c4nc(C5CCCC5)ccc34)cc12)N1CCCCC1] | 1600 |
| 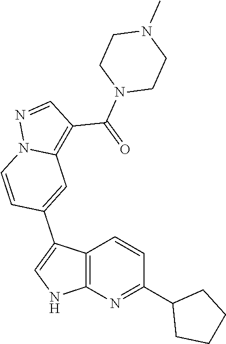 [SMILES: CN1CCN(C(=O)c2cnn3ccc(-c4c[nH]c5nc(C6CCCC6)ccc45)cc23)CC1] | 1601 |
| 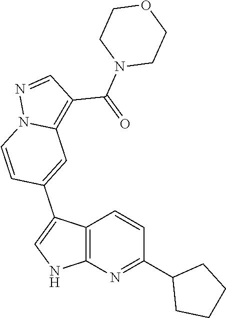 [SMILES: O=C(c1cnn2ccc(-c3c[nH]c4nc(C5CCCC5)ccc34)cc12)N1CCOCC1] | 1602 |
| 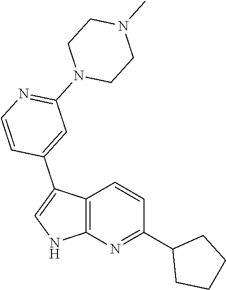 [SMILES: CN1CCN(c2cc(-c3c[nH]c4nc(C5CCCC5)ccc34)ccn2)CC1] | 1603 |
| 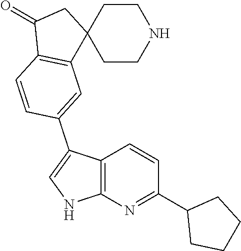 [SMILES: O=C1CC2(CCNCC2)c2cc(-c3c[nH]c4nc(C5CCCC5)ccc34)ccc21] | 1604 |
| 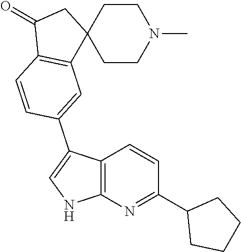 [SMILES: CN1CCC2(CC1)CC(=O)c1ccc(-c3c[nH]c4nc(C5CCCC5)ccc34)cc12] | 1605 |
| 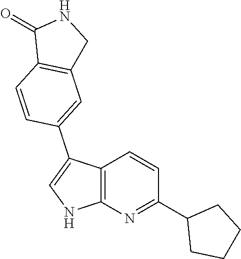 [SMILES: O=C1NCc2cc(-c3c[nH]c4nc(C5CCCC5)ccc34)ccc21] | 1606 |
| 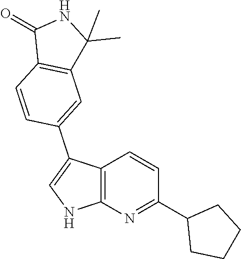 [SMILES: CC1(C)NC(=O)c2ccc(-c3c[nH]c4nc(C5CCCC5)ccc34)cc21] | 1607 |
| 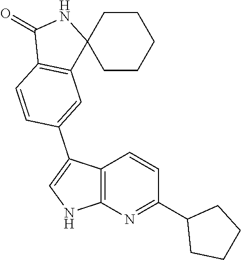 [SMILES: O=C1NC2(CCCCC2)c2cc(-c3c[nH]c4nc(C5CCCC5)ccc34)ccc21] | 1608 |
| 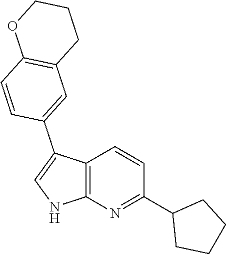 [SMILES: c1cc2c(cc1-c1c[nH]c3nc(C4CCCC4)ccc13)CCCO2] | 1609 |
| 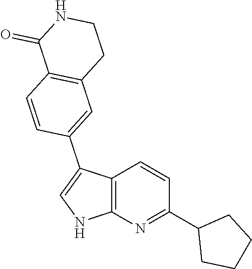 [SMILES: O=C1NCCc2cc(-c3c[nH]c4nc(C5CCCC5)ccc34)ccc21] | 1610 |
| 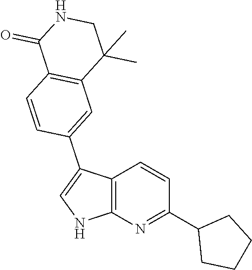 [SMILES: CC1(C)CNC(=O)c2ccc(-c3c[nH]c4nc(C5CCCC5)ccc34)cc21] | 1611 |
| 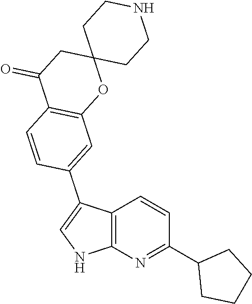 [SMILES: O=C1CC2(CCNCC2)Oc2cc(-c3c[nH]c4nc(C5CCCC5)ccc34)ccc21] | 1612 |
| 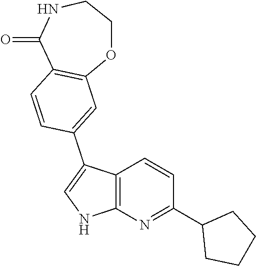 [SMILES: O=C1NCCOc2cc(-c3c[nH]c4nc(C5CCCC5)ccc34)ccc21] | 1613 |
| 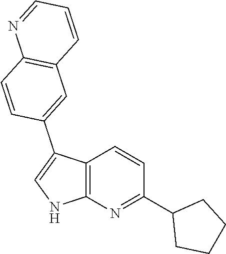 [SMILES: c1cnc2ccc(-c3c[nH]c4nc(C5CCCC5)ccc34)cc2c1] | 1614 |
| 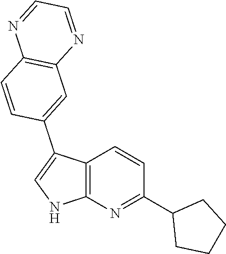 [SMILES: c1cnc2cc(-c3c[nH]c4nc(C5CCCC5)ccc34)ccc2n1] | 1615 |
| 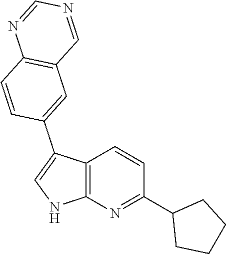 [SMILES: c1ncc2cc(-c3c[nH]c4nc(C5CCCC5)ccc34)ccc2n1] | 1616 |
| 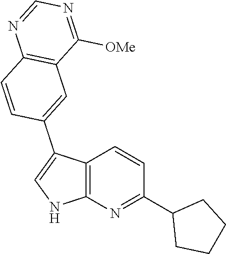 [SMILES: COc1ncnc2ccc(-c3c[nH]c4nc(C5CCCC5)ccc34)cc12] | 1617 |
| 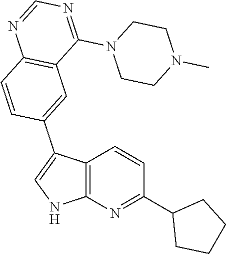 [SMILES: CN1CCN(c2ncnc3ccc(-c4c[nH]c5nc(C6CCCC6)ccc45)cc23)CC1] | 1618 |
| 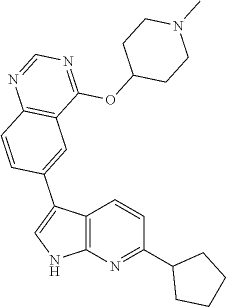 [SMILES: CN1CCC(Oc2ncnc3ccc(-c4c[nH]c5nc(C6CCCC6)ccc45)cc23)CC1] | 1619 |
| 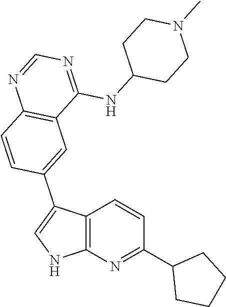 [SMILES: CN1CCC(Nc2ncnc3ccc(-c4c[nH]c5nc(C6CCCC6)ccc45)cc23)CC1] | 1620 |
| 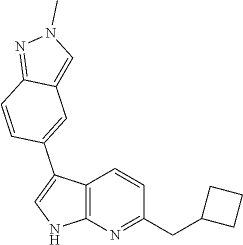 [SMILES: Cn1cc2cc(-c3c[nH]c4nc(CC5CCC5)ccc34)ccc2n1] | 1621 |
| 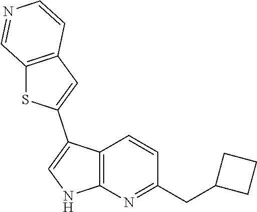 [SMILES: c1cc2cc(-c3c[nH]c4nc(CC5CCC5)ccc34)sc2cn1] | 1622 |
| 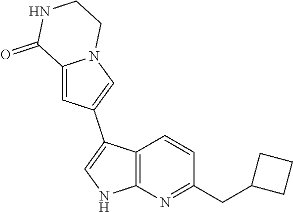 [SMILES: O=C1NCCn2cc(-c3c[nH]c4nc(CC5CCC5)ccc34)cc21] | 1623 |
| 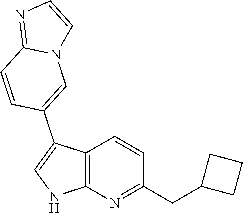 [SMILES: c1cn2cc(-c3c[nH]c4nc(CC5CCC5)ccc34)ccc2n1] | 1624 |
| 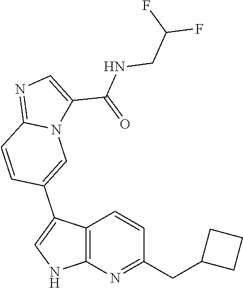 [SMILES: O=C(NCC(F)F)c1cnc2ccc(-c3c[nH]c4nc(CC5CCC5)ccc34)cn12] | 1625 |
| 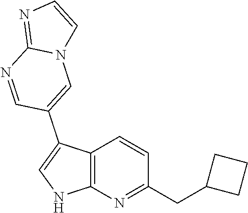 [SMILES: c1cn2cc(-c3c[nH]c4nc(CC5CCC5)ccc34)cnc2n1] | 1626 |
| 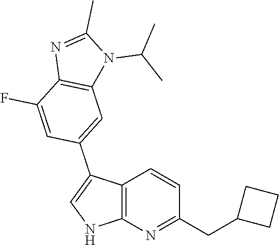 [SMILES: Cc1nc2c(F)cc(-c3c[nH]c4nc(CC5CCC5)ccc34)cc2n1C(C)C] | 1627 |
| 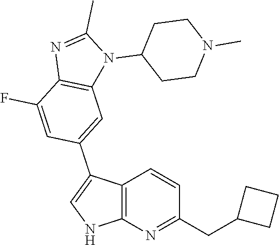 [SMILES: Cc1nc2c(F)cc(-c3c[nH]c4nc(CC5CCC5)ccc34)cc2n1C1CCN(C)CC1] | 1628 |
| 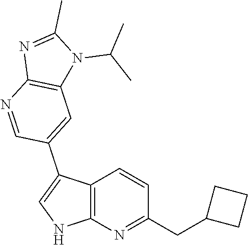 [SMILES: Cc1nc2ncc(-c3c[nH]c4nc(CC5CCC5)ccc34)cc2n1C(C)C] | 1629 |
| 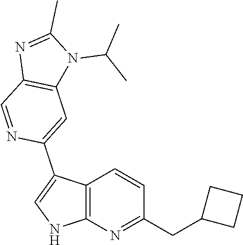 [SMILES: Cc1nc2cnc(-c3c[nH]c4nc(CC5CCC5)ccc34)cc2n1C(C)C] | 1630 |
| 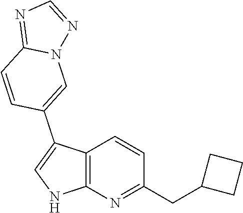 [SMILES: c1nc2ccc(-c3c[nH]c4nc(CC5CCC5)ccc34)cn2n1] | 1631 |
| 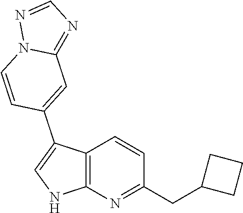 [SMILES: c1nc2cc(-c3c[nH]c4nc(CC5CCC5)ccc34)ccn2n1] | 1632 |
| 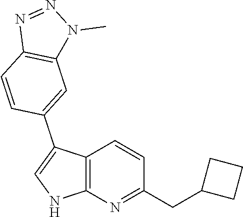 [SMILES: Cn1nnc2ccc(-c3c[nH]c4nc(CC5CCC5)ccc34)cc21] | 1633 |
| 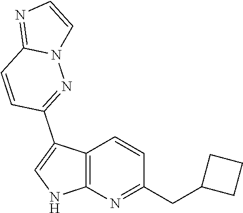 [SMILES: c1cn2nc(-c3c[nH]c4nc(CC5CCC5)ccc34)ccc2n1] | 1634 |
| 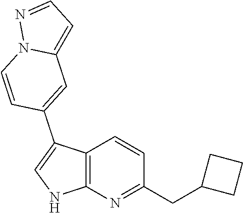 [SMILES: c1cc2cc(-c3c[nH]c4nc(CC5CCC5)ccc34)ccn2n1] | 1635 |
| 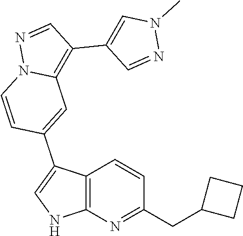 [SMILES: Cn1cc(-c2cnn3ccc(-c4c[nH]c5nc(CC6CCC6)ccc45)cc23)cn1] | 1636 |
| 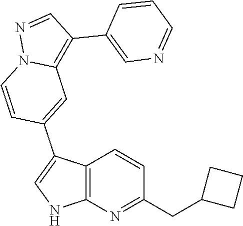 [SMILES: c1cncc(-c2cnn3ccc(-c4c[nH]c5nc(CC6CCC6)ccc45)cc23)c1] | 1637 |
| 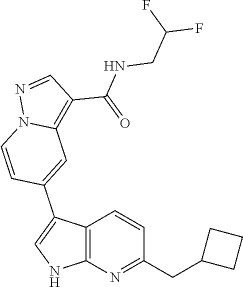 [SMILES: O=C(NCC(F)F)c1cnn2ccc(-c3c[nH]c4nc(CC5CCC5)ccc34)cc12] | 1638 |
| 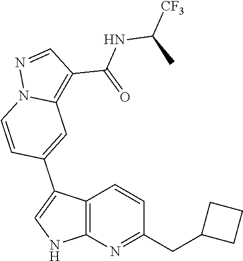 [SMILES: C[C@@H](NC(=O)c1cnn2ccc(-c3c[nH]c4nc(CC5CCC5)ccc34)cc12)C(F)(F)F] | 1639 |
| 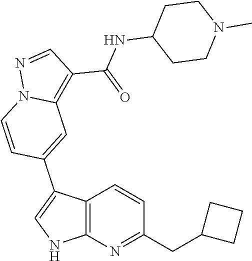 [SMILES: CN1CCC(NC(=O)c2cnn3ccc(-c4c[nH]c5nc(CC6CCC6)ccc45)cc23)CC1] | 1640 |
| 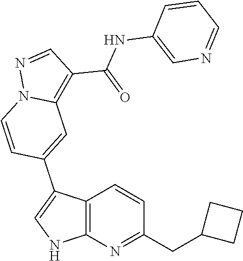 [SMILES: O=C(Nc1cccnc1)c1cnn2ccc(-c3c[nH]c4nc(CC5CCC5)ccc34)cc12] | 1641 |
| 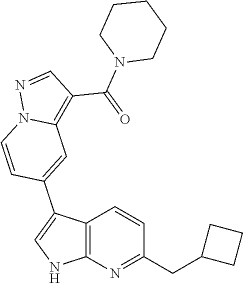 [SMILES: O=C(c1cnn2ccc(-c3c[nH]c4nc(CC5CCC5)ccc34)cc12)N1CCCCC1] | 1642 |
| 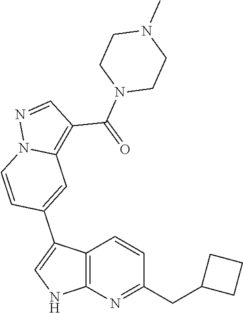 [SMILES: CN1CCN(C(=O)c2cnn3ccc(-c4c[nH]c5nc(CC6CCC6)ccc45)cc23)CC1] | 1643 |
| 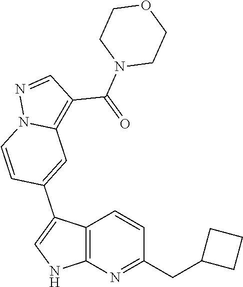 [SMILES: O=C(c1cnn2ccc(-c3c[nH]c4nc(CC5CCC5)ccc34)cc12)N1CCOCC1] | 1644 |
| 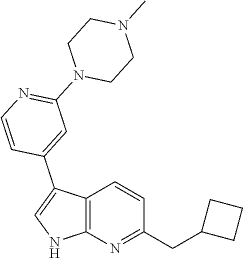 [SMILES: CN1CCN(c2cc(-c3c[nH]c4nc(CC5CCC5)ccc34)ccn2)CC1] | 1645 |
| 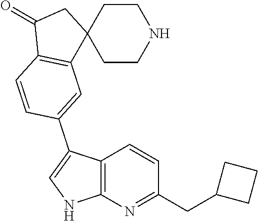 [SMILES: O=C1CC2(CCNCC2)c2cc(-c3c[nH]c4nc(CC5CCC5)ccc34)ccc21] | 1646 |
| 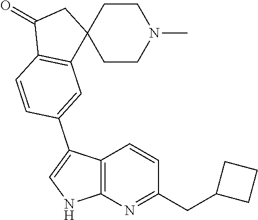 [SMILES: CN1CCC2(CC1)CC(=O)c1ccc(-c3c[nH]c4nc(CC5CCC5)ccc34)cc12] | 1647 |
| 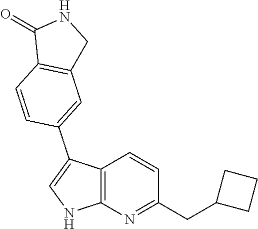 [SMILES: O=C1NCc2cc(-c3c[nH]c4nc(CC5CCC5)ccc34)ccc21] | 1648 |
| 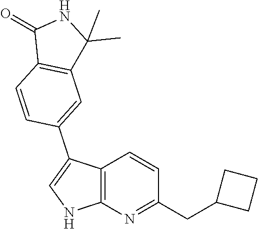 [SMILES: CC1(C)NC(=O)c2ccc(-c3c[nH]c4nc(CC5CCC5)ccc34)cc21] | 1649 |
| 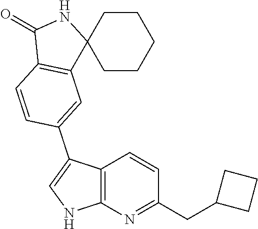 [SMILES: O=C1NC2(CCCCC2)c2cc(-c3c[nH]c4nc(CC5CCC5)ccc34)ccc21] | 1650 |
| 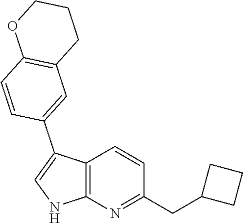 [SMILES: c1cc2c(cc1-c1c[nH]c3nc(CC4CCC4)ccc13)CCCO2] | 1651 |
| 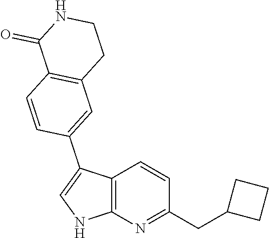 [SMILES: O=C1NCCc2cc(-c3c[nH]c4nc(CC5CCC5)ccc34)ccc21] | 1652 |
| 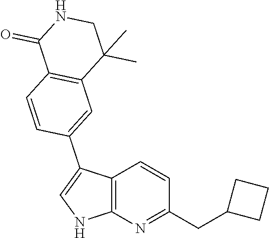 [SMILES: CC1(C)CNC(=O)c2ccc(-c3c[nH]c4nc(CC5CCC5)ccc34)cc21] | 1653 |
| 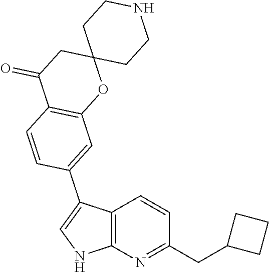 [SMILES: O=C1CC2(CCNCC2)Oc2cc(-c3c[nH]c4nc(CC5CCC5)ccc34)ccc21] | 1654 |
| 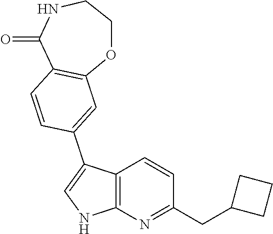 [SMILES: O=C1NCCOc2cc(-c3c[nH]c4nc(CC5CCC5)ccc34)ccc21] | 1655 |
| 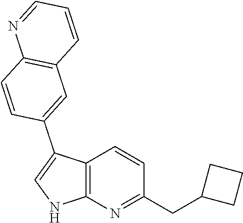 [SMILES: c1cnc2ccc(-c3c[nH]c4nc(CC5CCC5)ccc34)cc2c1] | 1656 |
| 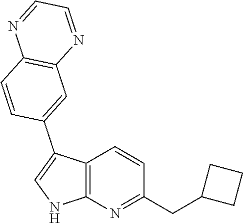 [SMILES: c1cnc2cc(-c3c[nH]c4nc(CC5CCC5)ccc34)ccc2n1] | 1657 |
| 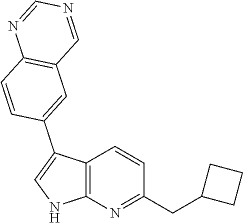 [SMILES: c1ncc2cc(-c3c[nH]c4nc(CC5CCC5)ccc34)ccc2n1] | 1658 |
| 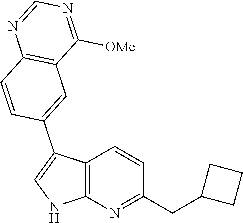 [SMILES: COc1ncnc2ccc(-c3c[nH]c4nc(CC5CCC5)ccc34)cc12] | 1659 |
| 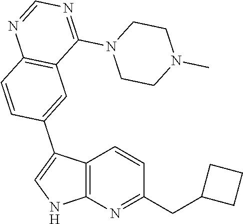 [SMILES: CN1CCN(c2ncnc3ccc(-c4c[nH]c5nc(CC6CCC6)ccc45)cc23)CC1] | 1660 |
| 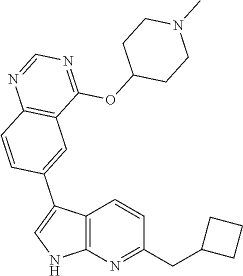 [SMILES: CN1CCC(Oc2ncnc3ccc(-c4c[nH]c5nc(CC6CCC6)ccc45)cc23)CC1] | 1661 |
| 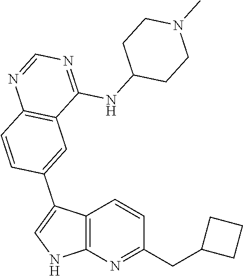 [SMILES: CN1CCC(Nc2ncnc3ccc(-c4c[nH]c5nc(CC6CCC6)ccc45)cc23)CC1] | 1662 |
| 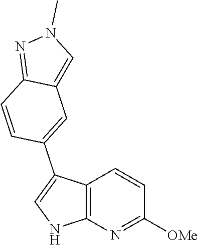 [SMILES: COc1ccc2c(-c3ccc4nn(C)cc4c3)c[nH]c2n1] | 1663 |
| 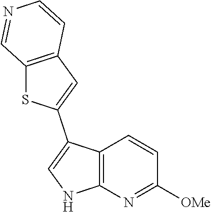 [SMILES: COc1ccc2c(-c3cc4ccncc4s3)c[nH]c2n1] | 1664 |
| 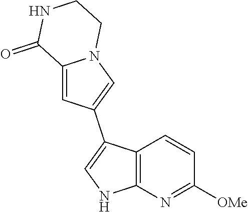 [SMILES: COc1ccc2c(-c3cc4n(c3)CCNC4=O)c[nH]c2n1] | 1665 |
| 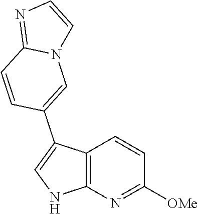 [SMILES: COc1ccc2c(-c3ccc4nccn4c3)c[nH]c2n1] | 1666 |
| 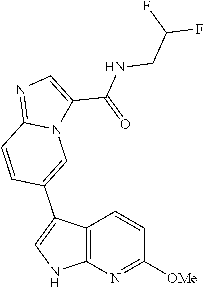 [SMILES: COc1ccc2c(-c3ccc4ncc(C(=O)NCC(F)F)n4c3)c[nH]c2n1] | 1667 |
| 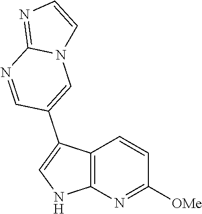 [SMILES: COc1ccc2c(-c3cnc4nccn4c3)c[nH]c2n1] | 1668 |
| 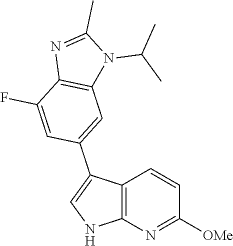 [SMILES: COc1ccc2c(-c3cc(F)c4nc(C)n(C(C)C)c4c3)c[nH]c2n1] | 1669 |
| 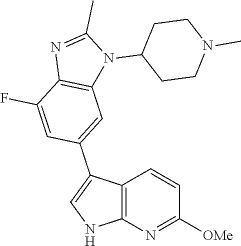 [SMILES: COc1ccc2c(-c3cc(F)c4nc(C)n(C5CCN(C)CC5)c4c3)c[nH]c2n1] | 1670 |
| 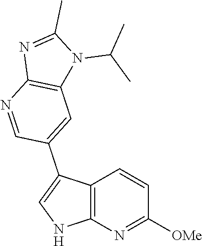 [SMILES: COc1ccc2c(-c3cnc4nc(C)n(C(C)C)c4c3)c[nH]c2n1] | 1671 |
| 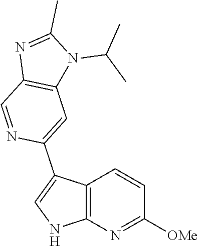 [SMILES: COc1ccc2c(-c3cc4c(cn3)nc(C)n4C(C)C)c[nH]c2n1] | 1672 |
| 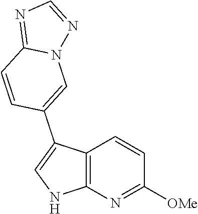 [SMILES: COc1ccc2c(-c3ccc4ncnn4c3)c[nH]c2n1] | 1673 |
| 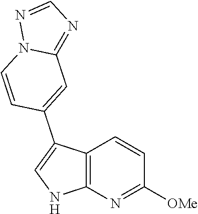 [SMILES: COc1ccc2c(-c3ccn4ncnc4c3)c[nH]c2n1] | 1674 |
| 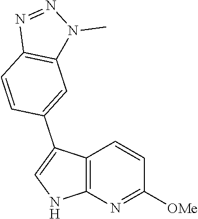 [SMILES: COc1ccc2c(-c3ccc4nnn(C)c4c3)c[nH]c2n1] | 1675 |
| 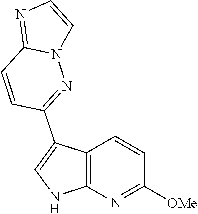 [SMILES: COc1ccc2c(-c3ccc4nccn4n3)c[nH]c2n1] | 1676 |
| 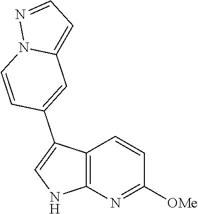 [SMILES: COc1ccc2c(-c3ccn4nccc4c3)c[nH]c2n1] | 1677 |
| 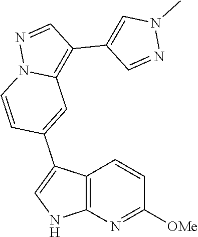 [SMILES: COc1ccc2c(-c3ccn4ncc(-c5cnn(C)c5)c4c3)c[nH]c2n1] | 1678 |
| 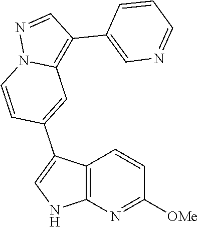 [SMILES: COc1ccc2c(-c3ccn4ncc(-c5cccnc5)c4c3)c[nH]c2n1] | 1679 |
| 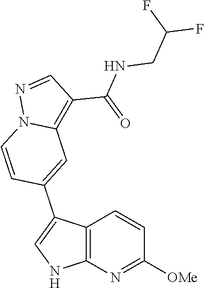 [SMILES: COc1ccc2c(-c3ccn4ncc(C(=O)NCC(F)F)c4c3)c[nH]c2n1] | 1680 |
| 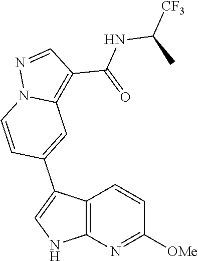 [SMILES: COc1ccc2c(-c3ccn4ncc(C(=O)N[C@H](C)C(F)(F)F)c4c3)c[nH]c2n1] | 1681 |
| 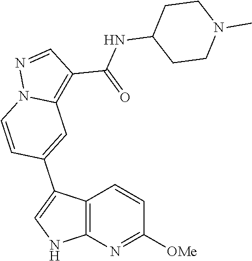 [SMILES: COc1ccc2c(-c3ccn4ncc(C(=O)NC5CCN(C)CC5)c4c3)c[nH]c2n1] | 1682 |
| 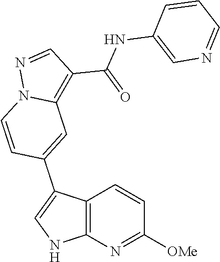 [SMILES: COc1ccc2c(-c3ccn4ncc(C(=O)Nc5cccnc5)c4c3)c[nH]c2n1] | 1683 |
| 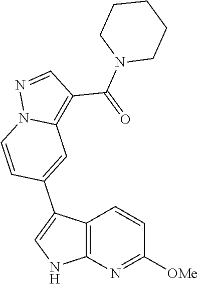 [SMILES: COc1ccc2c(-c3ccn4ncc(C(=O)N5CCCCC5)c4c3)c[nH]c2n1] | 1684 |
| 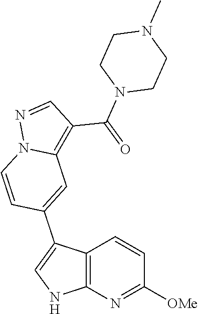 [SMILES: COc1ccc2c(-c3ccn4ncc(C(=O)N5CCN(C)CC5)c4c3)c[nH]c2n1] | 1685 |
| 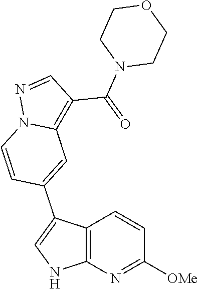 [SMILES: COc1ccc2c(-c3ccn4ncc(C(=O)N5CCOCC5)c4c3)c[nH]c2n1] | 1686 |
| 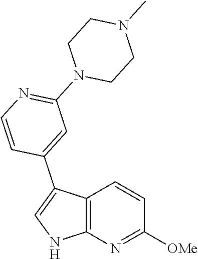 [SMILES: COc1ccc2c(-c3ccnc(N4CCN(C)CC4)c3)c[nH]c2n1] | 1687 |
| 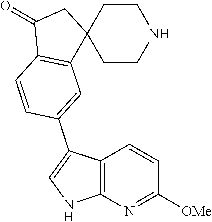 [SMILES: COc1ccc2c(-c3ccc4c(c3)C3(CCNCC3)CC4=O)c[nH]c2n1] | 1688 |
| 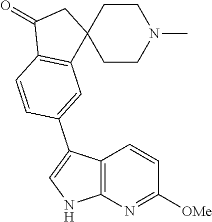 [SMILES: COc1ccc2c(-c3ccc4c(c3)C3(CCN(C)CC3)CC4=O)c[nH]c2n1] | 1689 |
| 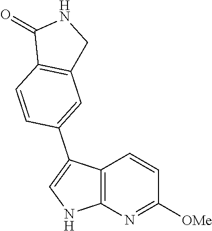 [SMILES: COc1ccc2c(-c3ccc4c(c3)CNC4=O)c[nH]c2n1] | 1690 |
| 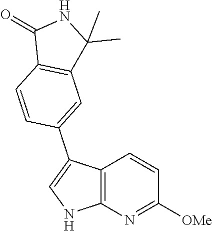 [SMILES: COc1ccc2c(-c3ccc4c(c3)C(C)(C)NC4=O)c[nH]c2n1] | 1691 |
| 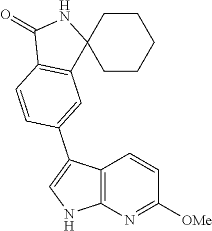 [SMILES: COc1ccc2c(-c3ccc4c(c3)C3(CCCCC3)NC4=O)c[nH]c2n1] | 1692 |
| 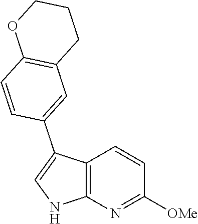 [SMILES: COc1ccc2c(-c3ccc4c(c3)CCCO4)c[nH]c2n1] | 1693 |
| 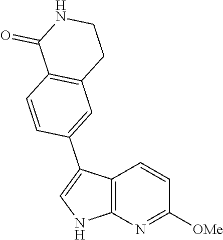 [SMILES: COc1ccc2c(-c3ccc4c(c3)CCNC4=O)c[nH]c2n1] | 1694 |
| 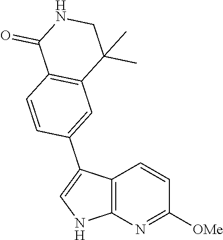 [SMILES: COc1ccc2c(-c3ccc4c(c3)C(C)(C)CNC4=O)c[nH]c2n1] | 1695 |
| 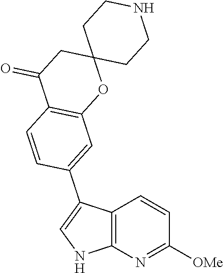 [SMILES: COc1ccc2c(-c3ccc4c(c3)OC3(CCNCC3)CC4=O)c[nH]c2n1] | 1696 |
| 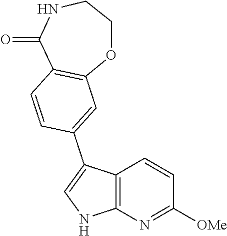 [SMILES: COc1ccc2c(-c3ccc4c(c3)OCCNC4=O)c[nH]c2n1] | 1697 |
| 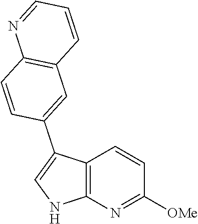 [SMILES: COc1ccc2c(-c3ccc4ncccc4c3)c[nH]c2n1] | 1698 |
| 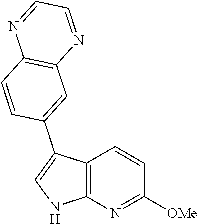 [SMILES: COc1ccc2c(-c3ccc4nccnc4c3)c[nH]c2n1] | 1699 |
| 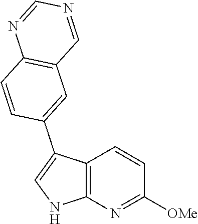 [SMILES: COc1ccc2c(-c3ccc4ncncc4c3)c[nH]c2n1] | 1700 |
| 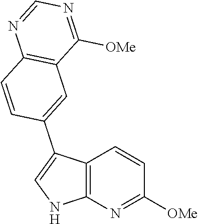 [SMILES: COc1ccc2c(-c3ccc4ncnc(OC)c4c3)c[nH]c2n1] | 1701 |
| 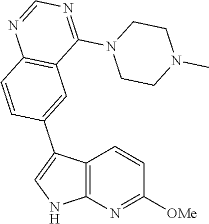 [SMILES: COc1ccc2c(-c3ccc4ncnc(N5CCN(C)CC5)c4c3)c[nH]c2n1] | 1702 |
| 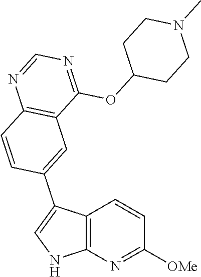 [SMILES: COc1ccc2c(-c3ccc4ncnc(OC5CCN(C)CC5)c4c3)c[nH]c2n1] | 1703 |
| 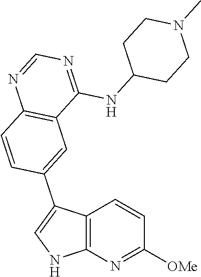 [SMILES: COc1ccc2c(-c3ccc4ncnc(NC5CCN(C)CC5)c4c3)c[nH]c2n1] | 1704 |
| 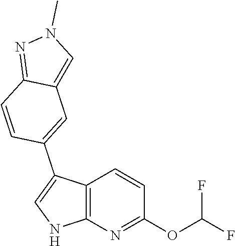 [SMILES: Cn1cc2cc(-c3c[nH]c4nc(OC(F)F)ccc34)ccc2n1] | 1705 |
| 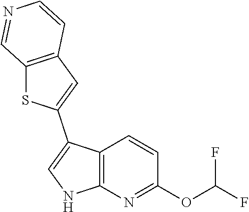 [SMILES: FC(F)Oc1ccc2c(-c3cc4ccncc4s3)c[nH]c2n1] | 1706 |
| 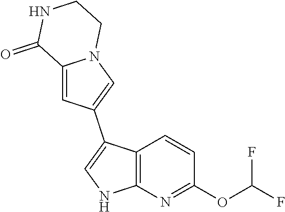 [SMILES: O=C1NCCn2cc(-c3c[nH]c4nc(OC(F)F)ccc34)cc21] | 1707 |
| 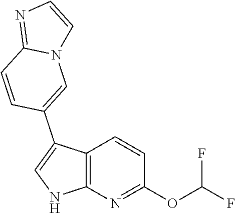 [SMILES: FC(F)Oc1ccc2c(-c3ccc4nccn4c3)c[nH]c2n1] | 1708 |
| 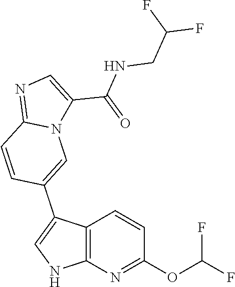 [SMILES: O=C(NCC(F)F)c1cnc2ccc(-c3c[nH]c4nc(OC(F)F)ccc34)cn12] | 1709 |
| 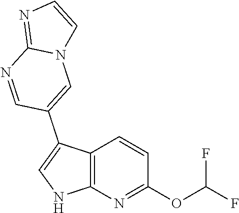 [SMILES: FC(F)Oc1ccc2c(-c3cnc4nccn4c3)c[nH]c2n1] | 1710 |
| 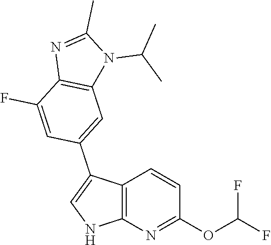 [SMILES: Cc1nc2c(F)cc(-c3c[nH]c4nc(OC(F)F)ccc34)cc2n1C(C)C] | 1711 |
| 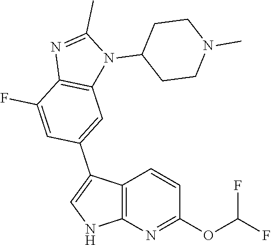 [SMILES: Cc1nc2c(F)cc(-c3c[nH]c4nc(OC(F)F)ccc34)cc2n1C1CCN(C)CC1] | 1712 |
| 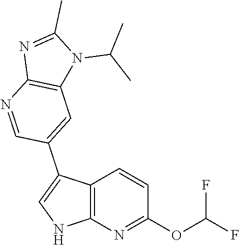 [SMILES: Cc1nc2ncc(-c3c[nH]c4nc(OC(F)F)ccc34)cc2n1C(C)C] | 1713 |
| 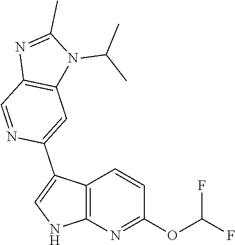 [SMILES: Cc1nc2cnc(-c3c[nH]c4nc(OC(F)F)ccc34)cc2n1C(C)C] | 1714 |
| 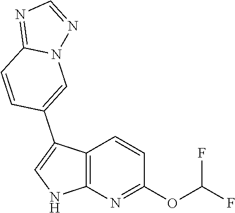 [SMILES: FC(F)Oc1ccc2c(-c3ccc4ncnn4c3)c[nH]c2n1] | 1715 |
| 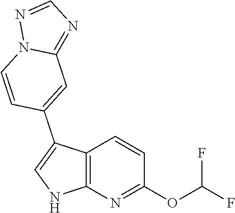 [SMILES: FC(F)Oc1ccc2c(-c3ccn4ncnc4c3)c[nH]c2n1] | 1716 |
| 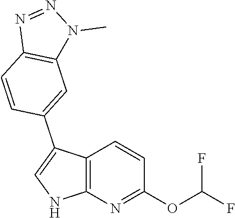 [SMILES: Cn1nnc2ccc(-c3c[nH]c4nc(OC(F)F)ccc34)cc21] | 1717 |
| 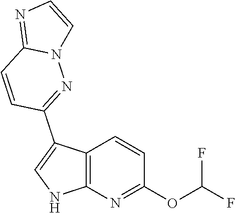 [SMILES: FC(F)Oc1ccc2c(-c3ccc4nccn4n3)c[nH]c2n1] | 1718 |
| 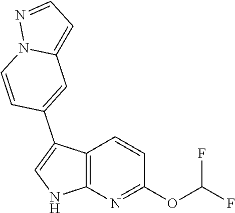 [SMILES: FC(F)Oc1ccc2c(-c3ccn4nccc4c3)c[nH]c2n1] | 1719 |
| 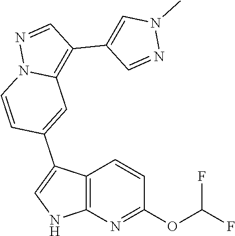 [SMILES: Cn1cc(-c2cnn3ccc(-c4c[nH]c5nc(OC(F)F)ccc45)cc23)cn1] | 1720 |
| 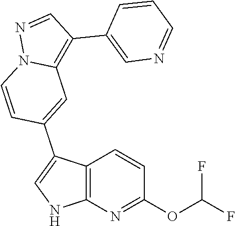 [SMILES: FC(F)Oc1ccc2c(-c3ccn4ncc(-c5cccnc5)c4c3)c[nH]c2n1] | 1721 |
| 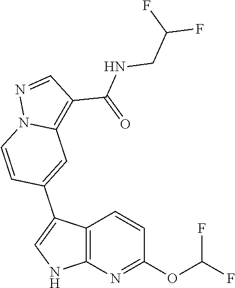 [SMILES: O=C(NCC(F)F)c1cnn2ccc(-c3c[nH]c4nc(OC(F)F)ccc34)cc12] | 1722 |
| 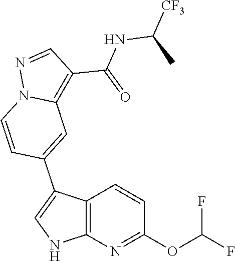 [SMILES: C[C@@H](NC(=O)c1cnn2ccc(-c3c[nH]c4nc(OC(F)F)ccc34)cc12)C(F)(F)F] | 1723 |
| 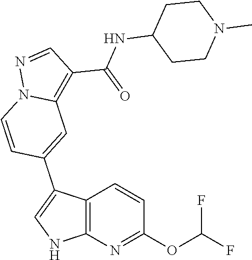 [SMILES: CN1CCC(NC(=O)c2cnn3ccc(-c4c[nH]c5nc(OC(F)F)ccc45)cc23)CC1] | 1724 |
| 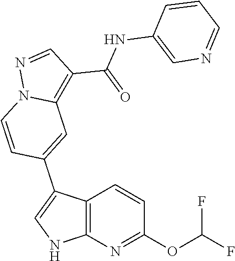 [SMILES: O=C(Nc1cccnc1)c1cnn2ccc(-c3c[nH]c4nc(OC(F)F)ccc34)cc12] | 1725 |
| 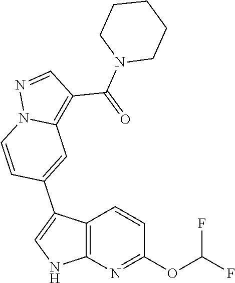 [SMILES: O=C(c1cnn2ccc(-c3c[nH]c4nc(OC(F)F)ccc34)cc12)N1CCCCC1] | 1726 |
| 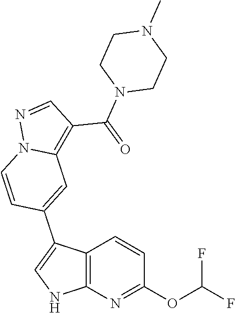 [SMILES: CN1CCN(C(=O)c2cnn3ccc(-c4c[nH]c5nc(OC(F)F)ccc45)cc23)CC1] | 1727 |
| 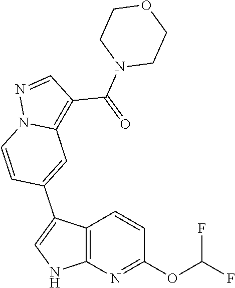 [SMILES: O=C(c1cnn2ccc(-c3c[nH]c4nc(OC(F)F)ccc34)cc12)N1CCOCC1] | 1728 |
| 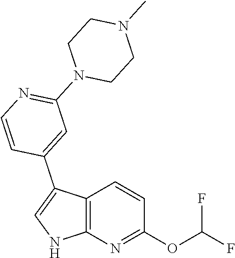 [SMILES: CN1CCN(c2cc(-c3c[nH]c4nc(OC(F)F)ccc34)ccn2)CC1] | 1729 |
| 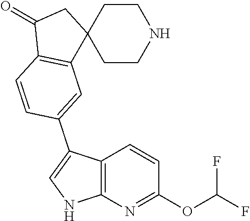 [SMILES: O=C1CC2(CCNCC2)c2cc(-c3c[nH]c4nc(OC(F)F)ccc34)ccc21] | 1730 |
| 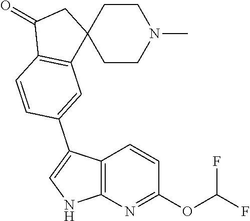 [SMILES: CN1CCC2(CC1)CC(=O)c1ccc(-c3c[nH]c4nc(OC(F)F)ccc34)cc12] | 1731 |
| 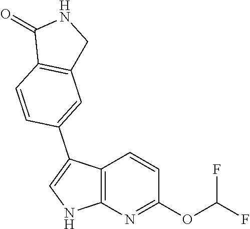 [SMILES: O=C1NCc2cc(-c3c[nH]c4nc(OC(F)F)ccc34)ccc21] | 1732 |
| 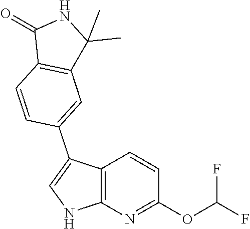 [SMILES: CC1(C)NC(=O)c2ccc(-c3c[nH]c4nc(OC(F)F)ccc34)cc21] | 1733 |
| 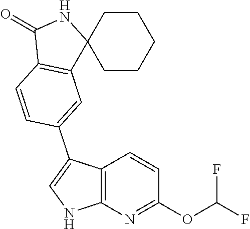 [SMILES: O=C1NC2(CCCCC2)c2cc(-c3c[nH]c4nc(OC(F)F)ccc34)ccc21] | 1734 |
| 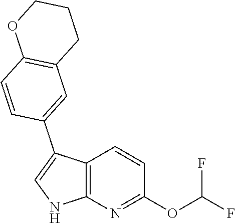 [SMILES: FC(F)Oc1ccc2c(-c3ccc4c(c3)CCCO4)c[nH]c2n1] | 1735 |
| 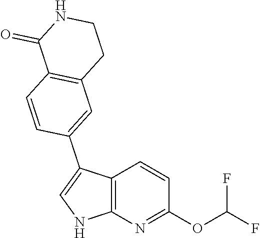 [SMILES: O=C1NCCc2cc(-c3c[nH]c4nc(OC(F)F)ccc34)ccc21] | 1736 |
| 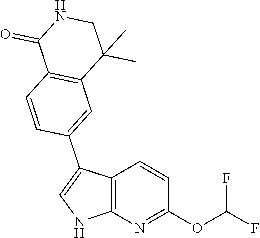 [SMILES: CC1(C)CNC(=O)c2ccc(-c3c[nH]c4nc(OC(F)F)ccc34)cc21] | 1737 |
| 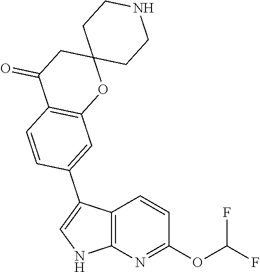 [SMILES: O=C1CC2(CCNCC2)Oc2cc(-c3c[nH]c4nc(OC(F)F)ccc34)ccc21] | 1738 |
| 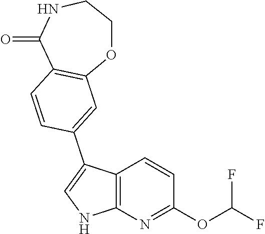 [SMILES: O=C1NCCOc2cc(-c3c[nH]c4nc(OC(F)F)ccc34)ccc21] | 1739 |
| 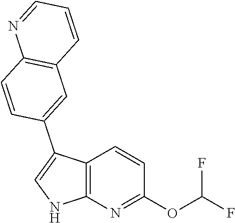 [SMILES: FC(F)Oc1ccc2c(-c3ccc4ncccc4c3)c[nH]c2n1] | 1740 |
| 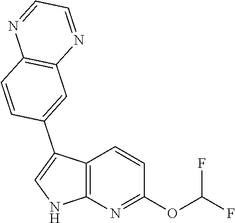 [SMILES: FC(F)Oc1ccc2c(-c3ccc4nccnc4c3)c[nH]c2n1] | 1741 |
| 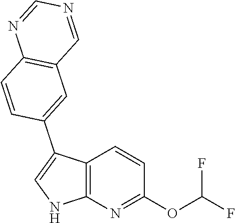 [SMILES: FC(F)Oc1ccc2c(-c3ccc4ncncc4c3)c[nH]c2n1] | 1742 |
| 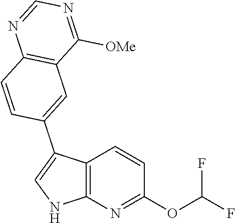 [SMILES: COc1ncnc2ccc(-c3c[nH]c4nc(OC(F)F)ccc34)cc12] | 1743 |
| 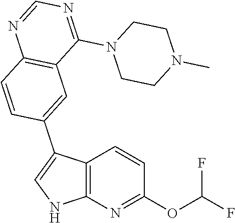 [SMILES: CN1CCN(c2ncnc3ccc(-c4c[nH]c5nc(OC(F)F)ccc45)cc23)CC1] | 1744 |
| 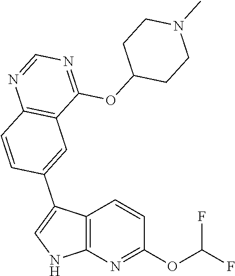 [SMILES: CN1CCC(Oc2ncnc3ccc(-c4c[nH]c5nc(OC(F)F)ccc45)cc23)CC1] | 1745 |
| 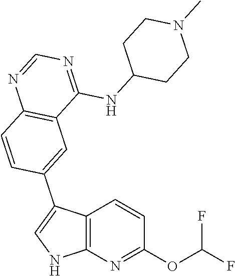 [SMILES: CN1CCC(Nc2ncnc3ccc(-c4c[nH]c5nc(OC(F)F)ccc45)cc23)CC1] | 1746 |
| 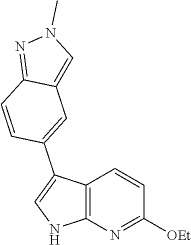 [SMILES: CCOc1ccc2c(-c3ccc4nn(C)cc4c3)c[nH]c2n1] | 1747 |
| 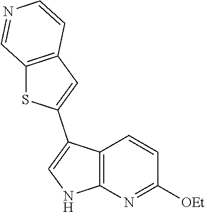 [SMILES: CCOc1ccc2c(-c3cc4ccncc4s3)c[nH]c2n1] | 1748 |
| 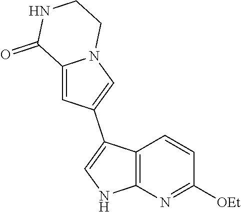 [SMILES: CCOc1ccc2c(-c3cc4n(c3)CCNC4=O)c[nH]c2n1] | 1749 |
| 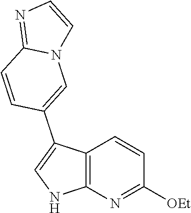 [SMILES: CCOc1ccc2c(-c3ccc4nccn4c3)c[nH]c2n1] | 1750 |
| 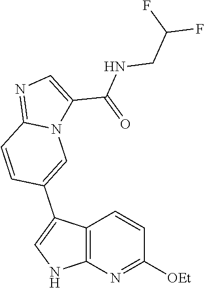 [SMILES: CCOc1ccc2c(-c3ccc4ncc(C(=O)NCC(F)F)n4c3)c[nH]c2n1] | 1751 |
| 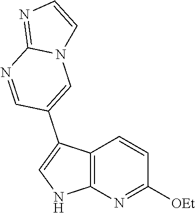 [SMILES: CCOc1ccc2c(-c3cnc4nccn4c3)c[nH]c2n1] | 1752 |
| 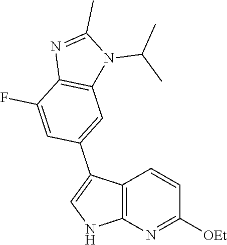 [SMILES: CCOc1ccc2c(-c3cc(F)c4nc(C)n(C(C)C)c4c3)c[nH]c2n1] | 1753 |
| 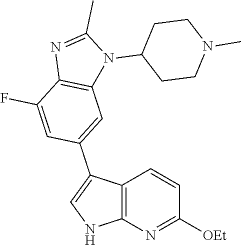 [SMILES: CCOc1ccc2c(-c3cc(F)c4nc(C)n(C5CCN(C)CC5)c4c3)c[nH]c2n1] | 1754 |
| 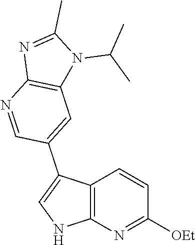 [SMILES: CCOc1ccc2c(-c3cnc4nc(C)n(C(C)C)c4c3)c[nH]c2n1] | 1755 |
| 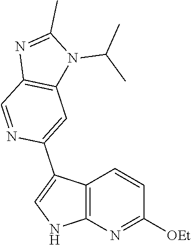 [SMILES: CCOc1ccc2c(-c3cc4c(cn3)nc(C)n4C(C)C)c[nH]c2n1] | 1756 |
| 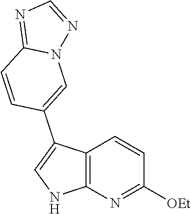 [SMILES: CCOc1ccc2c(-c3ccc4ncnn4c3)c[nH]c2n1] | 1757 |
| 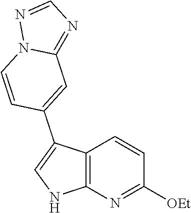 [SMILES: CCOc1ccc2c(-c3ccn4ncnc4c3)c[nH]c2n1] | 1758 |
| 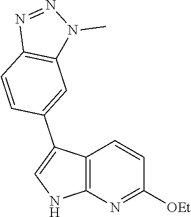 [SMILES: CCOc1ccc2c(-c3ccc4nnn(C)c4c3)c[nH]c2n1] | 1759 |
| 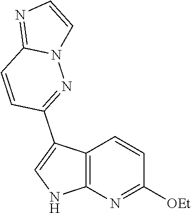 [SMILES: CCOc1ccc2c(-c3ccc4nccn4n3)c[nH]c2n1] | 1760 |
| 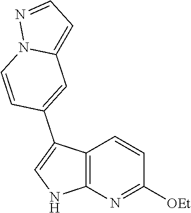 [SMILES: CCOc1ccc2c(-c3ccn4nccc4c3)c[nH]c2n1] | 1761 |
| 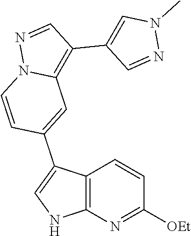 [SMILES: CCOc1ccc2c(-c3ccn4ncc(-c5cnn(C)c5)c4c3)c[nH]c2n1] | 1762 |
| 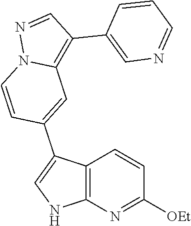 [SMILES: CCOc1ccc2c(-c3ccn4ncc(-c5cccnc5)c4c3)c[nH]c2n1] | 1763 |
| 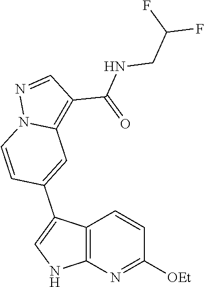 [SMILES: CCOc1ccc2c(-c3ccn4ncc(C(=O)NCC(F)F)c4c3)c[nH]c2n1] | 1764 |
| 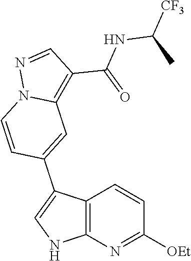 [SMILES: CCOc1ccc2c(-c3ccn4ncc(C(=O)N[C@H](C)C(F)(F)F)c4c3)c[nH]c2n1] | 1765 |
| 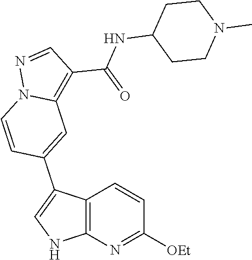 [SMILES: CCOc1ccc2c(-c3ccn4ncc(C(=O)NC5CCN(C)CC5)c4c3)c[nH]c2n1] | 1766 |
| 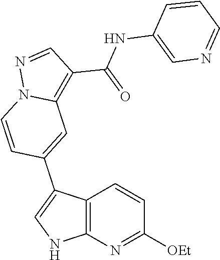 [SMILES: CCOc1ccc2c(-c3ccn4ncc(C(=O)Nc5cccnc5)c4c3)c[nH]c2n1] | 1767 |
| 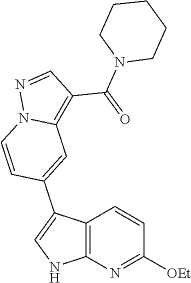 [SMILES: CCOc1ccc2c(-c3ccn4ncc(C(=O)N5CCCCC5)c4c3)c[nH]c2n1] | 1768 |
| 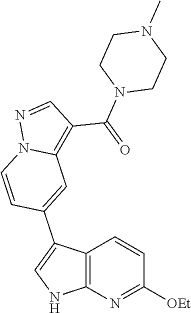 [SMILES: CCOc1ccc2c(-c3ccn4ncc(C(=O)N5CCN(C)CC5)c4c3)c[nH]c2n1] | 1769 |
| 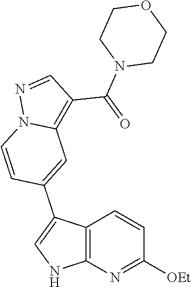 [SMILES: CCOc1ccc2c(-c3ccn4ncc(C(=O)N5CCOCC5)c4c3)c[nH]c2n1] | 1770 |
| 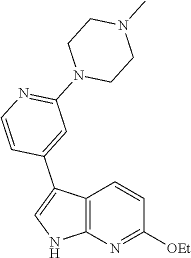 [SMILES: CCOc1ccc2c(-c3ccnc(N4CCN(C)CC4)c3)c[nH]c2n1] | 1771 |
| 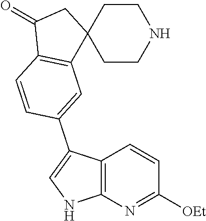 [SMILES: CCOc1ccc2c(-c3ccc4c(c3)C3(CCNCC3)CC4=O)c[nH]c2n1] | 1772 |
| 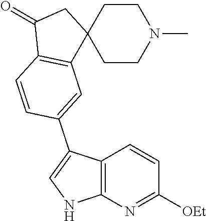 [SMILES: CCOc1ccc2c(-c3ccc4c(c3)C3(CCN(C)CC3)CC4=O)c[nH]c2n1] | 1773 |
| 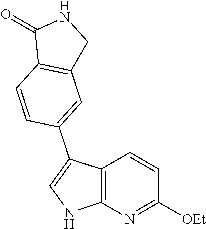 [SMILES: CCOc1ccc2c(-c3ccc4c(c3)CNC4=O)c[nH]c2n1] | 1774 |
| 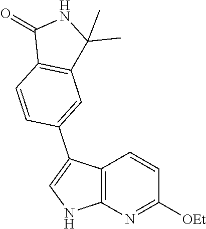 [SMILES: CCOc1ccc2c(-c3ccc4c(c3)C(C)(C)NC4=O)c[nH]c2n1] | 1775 |
| 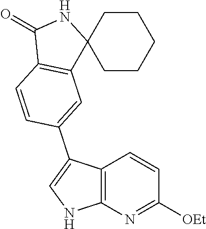 [SMILES: CCOc1ccc2c(-c3ccc4c(c3)C3(CCCCC3)NC4=O)c[nH]c2n1] | 1776 |
| 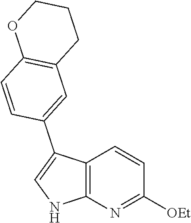 [SMILES: CCOc1ccc2c(-c3ccc4c(c3)CCCO4)c[nH]c2n1] | 1777 |
| 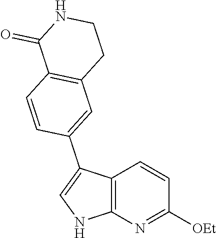 [SMILES: CCOc1ccc2c(-c3ccc4c(c3)CCNC4=O)c[nH]c2n1] | 1778 |
| 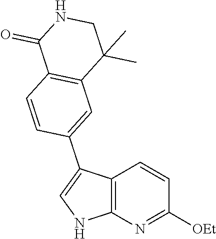 [SMILES: CCOc1ccc2c(-c3ccc4c(c3)C(C)(C)CNC4=O)c[nH]c2n1] | 1779 |
| 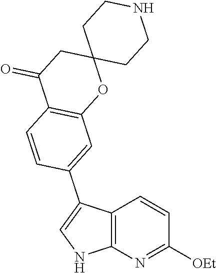 [SMILES: CCOc1ccc2c(-c3ccc4c(c3)OC3(CCNCC3)CC4=O)c[nH]c2n1] | 1780 |
| 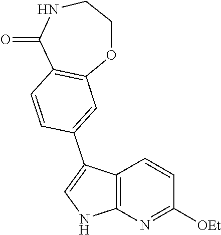 [SMILES: CCOc1ccc2c(-c3ccc4c(c3)OCCNC4=O)c[nH]c2n1] | 1781 |
| 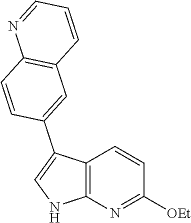 [SMILES: CCOc1ccc2c(-c3ccc4ncccc4c3)c[nH]c2n1] | 1782 |
| 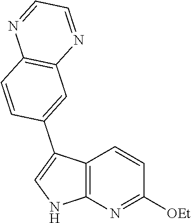 [SMILES: CCOc1ccc2c(-c3ccc4nccnc4c3)c[nH]c2n1] | 1783 |
| 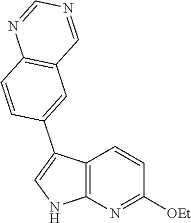 [SMILES: CCOc1ccc2c(-c3ccc4ncncc4c3)c[nH]c2n1] | 1784 |
| 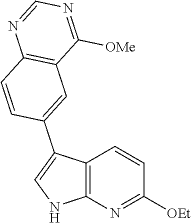 [SMILES: CCOc1ccc2c(-c3ccc4ncnc(OC)c4c3)c[nH]c2n1] | 1785 |
| 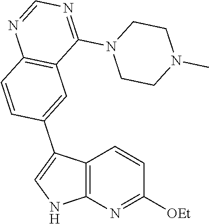 [SMILES: CCOc1ccc2c(-c3ccc4ncnc(N5CCN(C)CC5)c4c3)c[nH]c2n1] | 1786 |
| 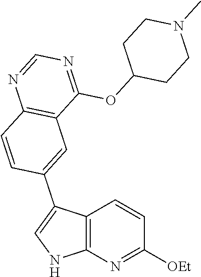 [SMILES: CCOc1ccc2c(-c3ccc4ncnc(OC5CCN(C)CC5)c4c3)c[nH]c2n1] | 1787 |
| 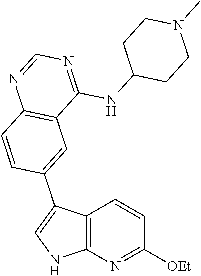 [SMILES: CCOc1ccc2c(-c3ccc4ncnc(NC5CCN(C)CC5)c4c3)c[nH]c2n1] | 1788 |
| 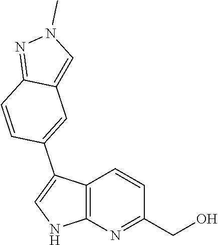 [SMILES: Cn1cc2cc(-c3c[nH]c4nc(CO)ccc34)ccc2n1] | 1789 |
| 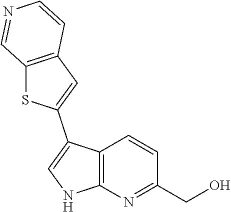 [SMILES: OCc1ccc2c(-c3cc4ccncc4s3)c[nH]c2n1] | 1790 |
| 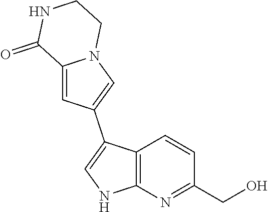 [SMILES: O=C1NCCn2cc(-c3c[nH]c4nc(CO)ccc34)cc21] | 1791 |
| 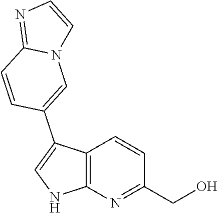 [SMILES: OCc1ccc2c(-c3ccc4nccn4c3)c[nH]c2n1] | 1792 |
| 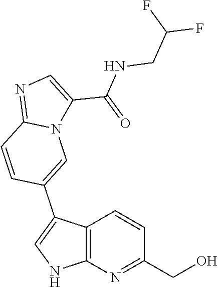 [SMILES: O=C(NCC(F)F)c1cnc2ccc(-c3c[nH]c4nc(CO)ccc34)cn12] | 1793 |
| 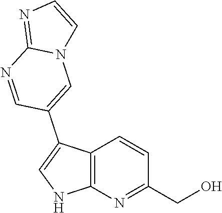 [SMILES: OCc1ccc2c(-c3cnc4nccn4c3)c[nH]c2n1] | 1794 |
| 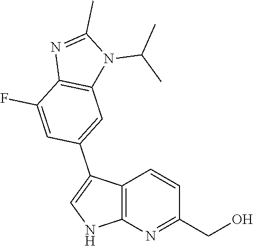 [SMILES: Cc1nc2c(F)cc(-c3c[nH]c4nc(CO)ccc34)cc2n1C(C)C] | 1795 |
| 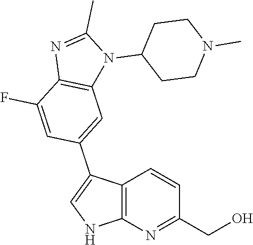 [SMILES: Cc1nc2c(F)cc(-c3c[nH]c4nc(CO)ccc34)cc2n1C1CCN(C)CC1] | 1796 |
| 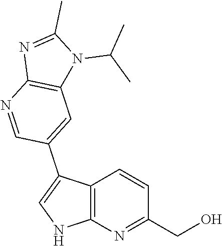 [SMILES: Cc1nc2ncc(-c3c[nH]c4nc(CO)ccc34)cc2n1C(C)C] | 1797 |
| 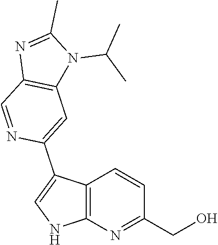 [SMILES: Cc1nc2cnc(-c3c[nH]c4nc(CO)ccc34)cc2n1C(C)C] | 1798 |
| 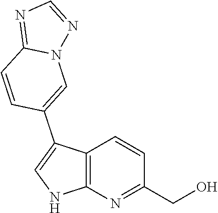 [SMILES: OCc1ccc2c(-c3ccc4ncnn4c3)c[nH]c2n1] | 1799 |
| 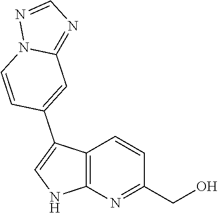 [SMILES: OCc1ccc2c(-c3ccn4ncnc4c3)c[nH]c2n1] | 1800 |
| 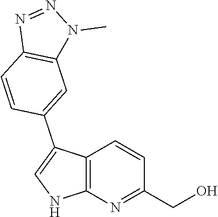 [SMILES: Cn1nnc2ccc(-c3c[nH]c4nc(CO)ccc34)cc21] | 1801 |
| 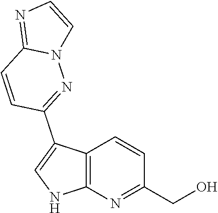 [SMILES: OCc1ccc2c(-c3ccc4nccn4n3)c[nH]c2n1] | 1802 |
| 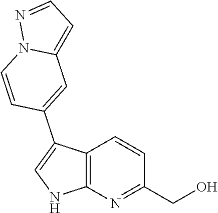 [SMILES: OCc1ccc2c(-c3ccn4nccc4c3)c[nH]c2n1] | 1803 |
| 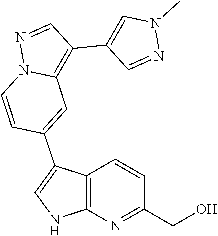 [SMILES: Cn1cc(-c2cnn3ccc(-c4c[nH]c5nc(CO)ccc45)cc23)cn1] | 1804 |
| 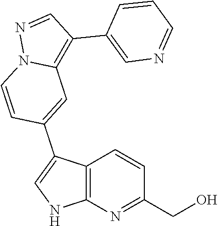 [SMILES: OCc1ccc2c(-c3ccn4ncc(-c5cccnc5)c4c3)c[nH]c2n1] | 1805 |
| 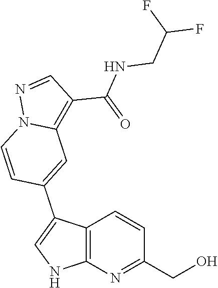 [SMILES: O=C(NCC(F)F)c1cnn2ccc(-c3c[nH]c4nc(CO)ccc34)cc12] | 1806 |
| 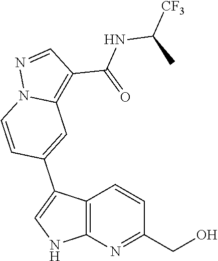 [SMILES: C[C@@H](NC(=O)c1cnn2ccc(-c3c[nH]c4nc(CO)ccc34)cc12)C(F)(F)F] | 1807 |
| 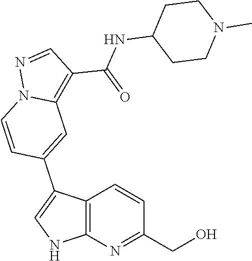 [SMILES: CN1CCC(NC(=O)c2cnn3ccc(-c4c[nH]c5nc(CO)ccc45)cc23)CC1] | 1808 |
| 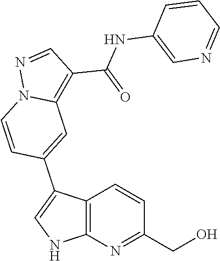 [SMILES: O=C(Nc1cccnc1)c1cnn2ccc(-c3c[nH]c4nc(CO)ccc34)cc12] | 1809 |
| 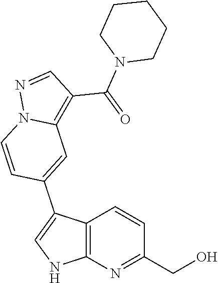 [SMILES: O=C(c1cnn2ccc(-c3c[nH]c4nc(CO)ccc34)cc12)N1CCCCC1] | 1810 |
| 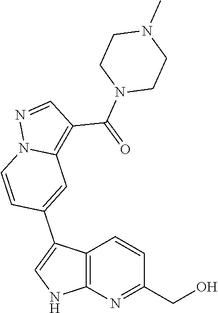 [SMILES: CN1CCN(C(=O)c2cnn3ccc(-c4c[nH]c5nc(CO)ccc45)cc23)CC1] | 1811 |
| 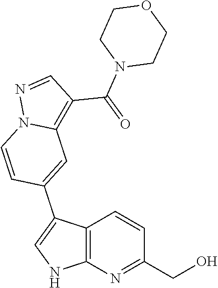 [SMILES: O=C(c1cnn2ccc(-c3c[nH]c4nc(CO)ccc34)cc12)N1CCOCC1] | 1812 |
| 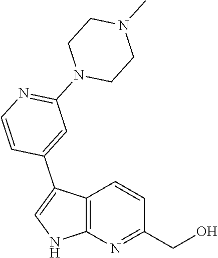 [SMILES: CN1CCN(c2cc(-c3c[nH]c4nc(CO)ccc34)ccn2)CC1] | 1813 |
| 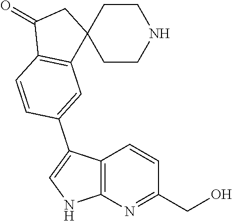 [SMILES: O=C1CC2(CCNCC2)c2cc(-c3c[nH]c4nc(CO)ccc34)ccc21] | 1814 |
| 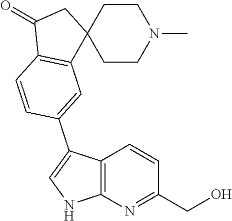 [SMILES: CN1CCC2(CC1)CC(=O)c1ccc(-c3c[nH]c4nc(CO)ccc34)cc12] | 1815 |
| 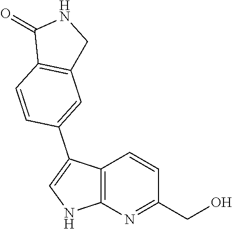 [SMILES: O=C1NCc2cc(-c3c[nH]c4nc(CO)ccc34)ccc21] | 1816 |
| 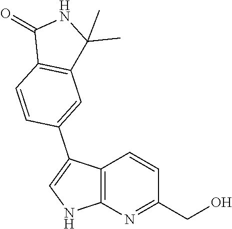 [SMILES: CC1(C)NC(=O)c2ccc(-c3c[nH]c4nc(CO)ccc34)cc21] | 1817 |
| 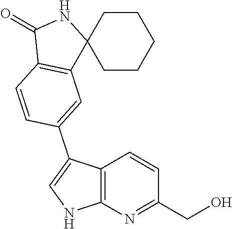 [SMILES: O=C1NC2(CCCCC2)c2cc(-c3c[nH]c4nc(CO)ccc34)ccc21] | 1818 |
| 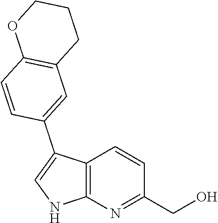 [SMILES: OCc1ccc2c(-c3ccc4c(c3)CCCO4)c[nH]c2n1] | 1819 |
| 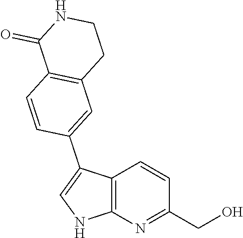 [SMILES: O=C1NCCc2cc(-c3c[nH]c4nc(CO)ccc34)ccc21] | 1820 |
| 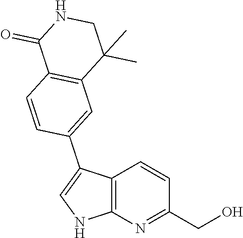 [SMILES: CC1(C)CNC(=O)c2ccc(-c3c[nH]c4nc(CO)ccc34)cc21] | 1821 |
| 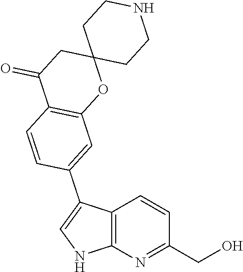 [SMILES: O=C1CC2(CCNCC2)Oc2cc(-c3c[nH]c4nc(CO)ccc34)ccc21] | 1822 |
| 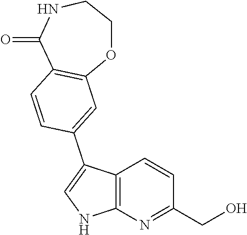 [SMILES: O=C1NCCOc2cc(-c3c[nH]c4nc(CO)ccc34)ccc21] | 1823 |
| 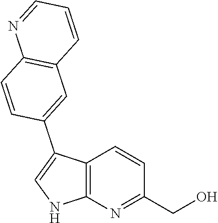 [SMILES: OCc1ccc2c(-c3ccc4ncccc4c3)c[nH]c2n1] | 1824 |
| 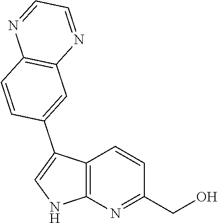 [SMILES: OCc1ccc2c(-c3ccc4nccnc4c3)c[nH]c2n1] | 1825 |
| 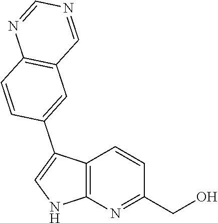 [SMILES: OCc1ccc2c(-c3ccc4ncncc4c3)c[nH]c2n1] | 1826 |
| 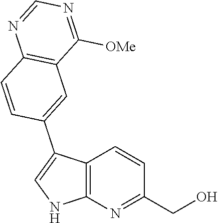 [SMILES: COc1ncnc2ccc(-c3c[nH]c4nc(CO)ccc34)cc12] | 1827 |
| 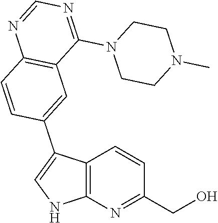 [SMILES: CN1CCN(c2ncnc3ccc(-c4c[nH]c5nc(CO)ccc45)cc23)CC1] | 1828 |
| 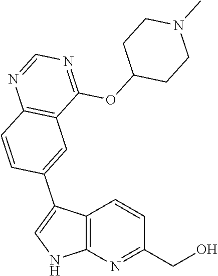 [SMILES: CN1CCC(Oc2ncnc3ccc(-c4c[nH]c5nc(CO)ccc45)cc23)CC1] | 1829 |
| 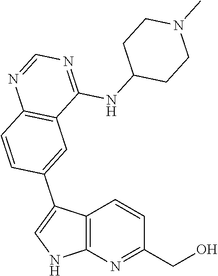 [SMILES: CN1CCC(Nc2ncnc3ccc(-c4c[nH]c5nc(CO)ccc45)cc23)CC1] | 1830 |
| 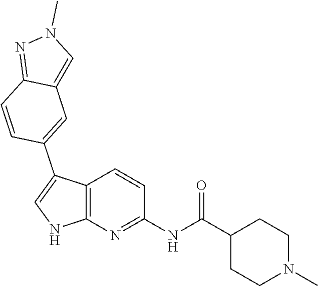 [SMILES: CN1CCC(C(=O)Nc2ccc3c(-c4ccc5nn(C)cc5c4)c[nH]c3n2)CC1] | 1831 |
| 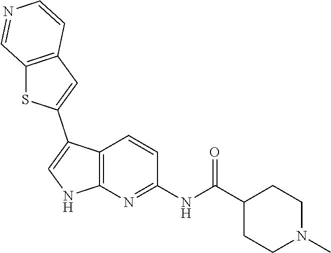 [SMILES: CN1CCC(C(=O)Nc2ccc3c(-c4cc5ccncc5s4)c[nH]c3n2)CC1] | 1832 |
| 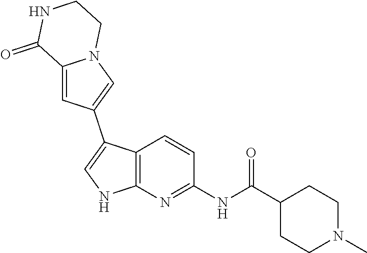 [SMILES: CN1CCC(C(=O)Nc2ccc3c(-c4cc5n(c4)CCNC5=O)c[nH]c3n2)CC1] | 1833 |
| 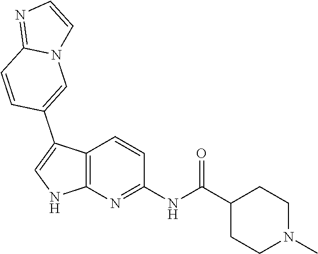 [SMILES: CN1CCC(C(=O)Nc2ccc3c(-c4ccc5nccn5c4)c[nH]c3n2)CC1] | 1834 |
| 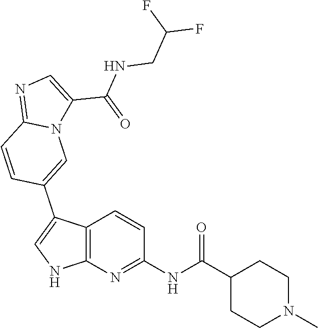 [SMILES: CN1CCC(C(=O)Nc2ccc3c(-c4ccc5ncc(C(=O)NCC(F)F)n5c4)c[nH]c3n2)CC1] | 1835 |
| 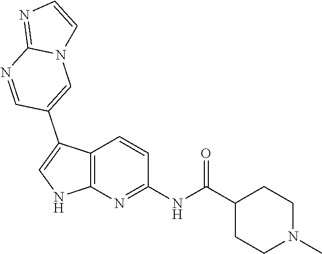 [SMILES: CN1CCC(C(=O)Nc2ccc3c(-c4cnc5nccn5c4)c[nH]c3n2)CC1] | 1836 |
| 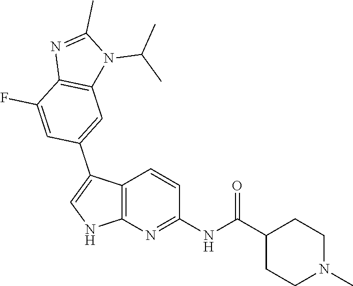 [SMILES: Cc1nc2c(F)cc(-c3c[nH]c4nc(NC(=O)C5CCN(C)CC5)ccc34)cc2n1C(C)C] | 1837 |
| 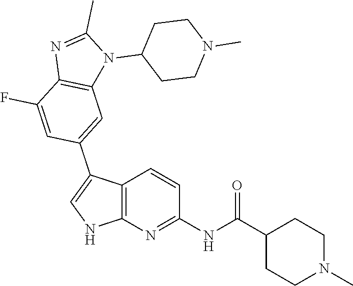 [SMILES: Cc1nc2c(F)cc(-c3c[nH]c4nc(NC(=O)C5CCN(C)CC5)ccc34)cc2n1C1CCN(C)CC1] | 1838 |
| 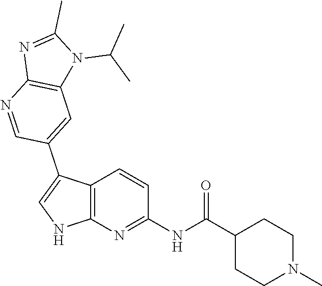 [SMILES: Cc1nc2ncc(-c3c[nH]c4nc(NC(=O)C5CCN(C)CC5)ccc34)cc2n1C(C)C] | 1839 |
| 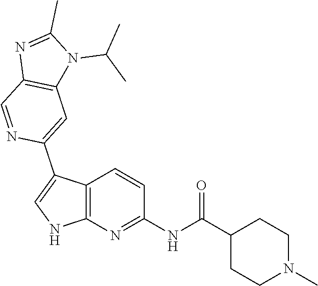 [SMILES: Cc1nc2cnc(-c3c[nH]c4nc(NC(=O)C5CCN(C)CC5)ccc34)cc2n1C(C)C] | 1840 |
| 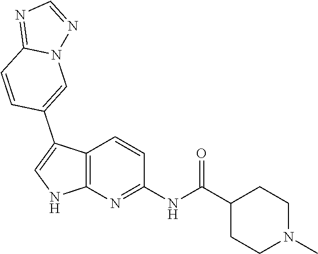 [SMILES: CN1CCC(C(=O)Nc2ccc3c(-c4ccc5ncnn5c4)c[nH]c3n2)CC1] | 1841 |
| 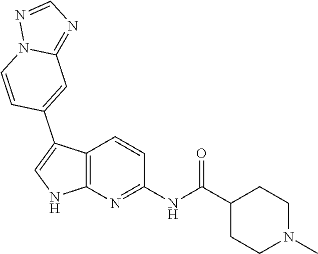 [SMILES: CN1CCC(C(=O)Nc2ccc3c(-c4ccn5ncnc5c4)c[nH]c3n2)CC1] | 1842 |
| 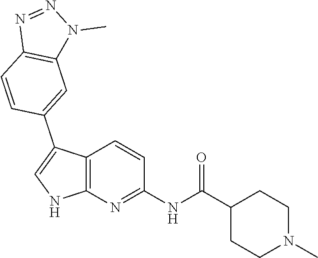 [SMILES: CN1CCC(C(=O)Nc2ccc3c(-c4ccc5nnn(C)c5c4)c[nH]c3n2)CC1] | 1843 |
| 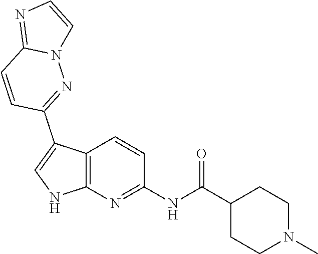 [SMILES: CN1CCC(C(=O)Nc2ccc3c(-c4ccc5nccn5n4)c[nH]c3n2)CC1] | 1844 |
| 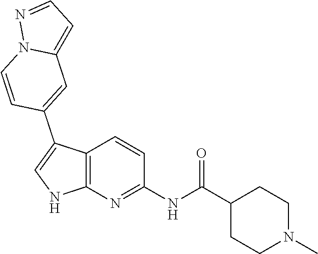 [SMILES: CN1CCC(C(=O)Nc2ccc3c(-c4ccn5nccc5c4)c[nH]c3n2)CC1] | 1845 |
| 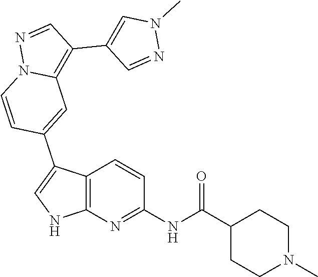 [SMILES: CN1CCC(C(=O)Nc2ccc3c(-c4ccn5ncc(-c6cnn(C)c6)c5c4)c[nH]c3n2)CC1] | 1846 |
| 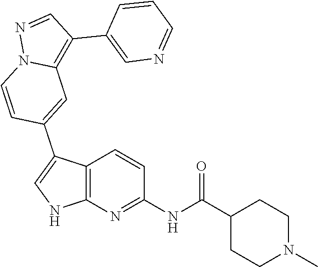 [SMILES: CN1CCC(C(=O)Nc2ccc3c(-c4ccn5ncc(-c6cccnc6)c5c4)c[nH]c3n2)CC1] | 1847 |
| 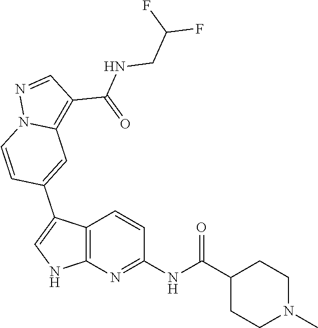 [SMILES: CN1CCC(C(=O)Nc2ccc3c(-c4ccn5ncc(C(=O)NCC(F)F)c5c4)c[nH]c3n2)CC1] | 1848 |
| 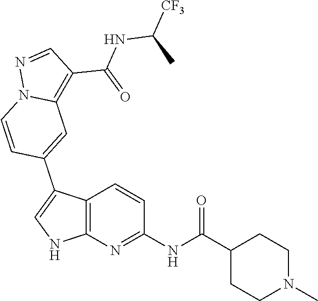 [SMILES: C[C@@H](NC(=O)c1cnn2ccc(-c3c[nH]c4nc(NC(=O)C5CCN(C)CC5)ccc34)cc12)C(F)(F)F] | 1849 |
| 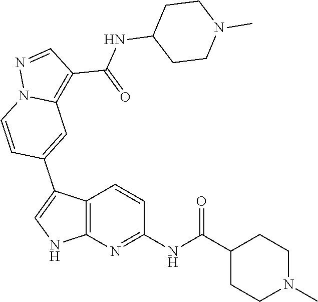 [SMILES: CN1CCC(NC(=O)c2cnn3ccc(-c4c[nH]c5nc(NC(=O)C6CCN(C)CC6)ccc45)cc23)CC1] | 1850 |
| 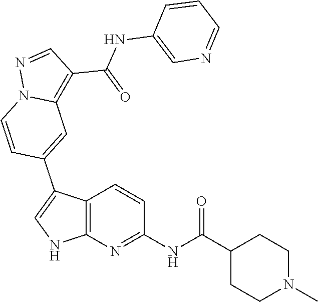 [SMILES: CN1CCC(C(=O)Nc2ccc3c(-c4ccn5ncc(C(=O)Nc6cccnc6)c5c4)c[nH]c3n2)CC1] | 1851 |
| 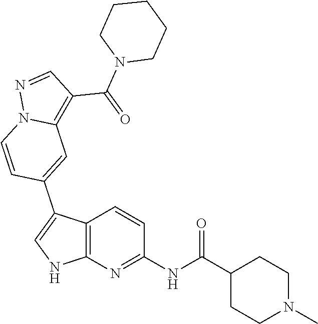 [SMILES: CN1CCC(C(=O)Nc2ccc3c(-c4ccn5ncc(C(=O)N6CCCCC6)c5c4)c[nH]c3n2)CC1] | 1852 |
| 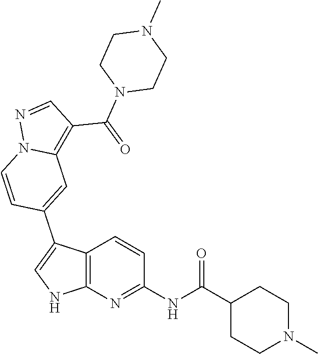 [SMILES: CN1CCC(C(=O)Nc2ccc3c(-c4ccn5ncc(C(=O)N6CCN(C)CC6)c5c4)c[nH]c3n2)CC1] | 1853 |
| 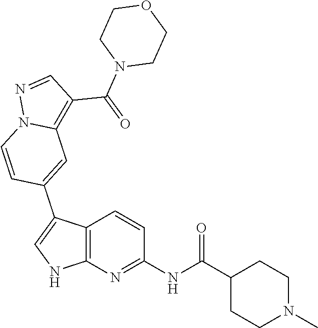 [SMILES: CN1CCC(C(=O)Nc2ccc3c(-c4ccn5ncc(C(=O)N6CCOCC6)c5c4)c[nH]c3n2)CC1] | 1854 |
| 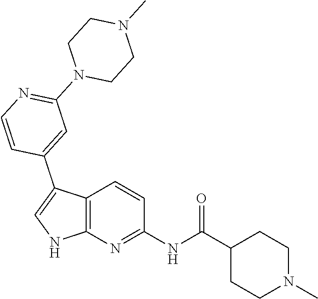 [SMILES: CN1CCC(C(=O)Nc2ccc3c(-c4ccnc(N5CCN(C)CC5)c4)c[nH]c3n2)CC1] | 1855 |
| 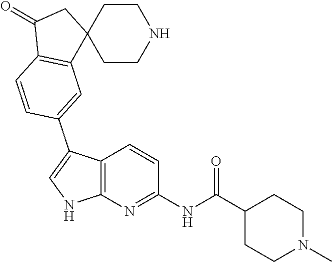 [SMILES: CN1CCC(C(=O)Nc2ccc3c(-c4ccc5c(c4)C4(CCNCC4)CC5=O)c[nH]c3n2)CC1] | 1856 |
| 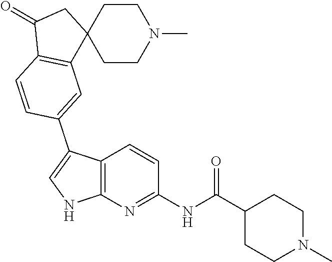 [SMILES: CN1CCC(C(=O)Nc2ccc3c(-c4ccc5c(c4)C4(CCN(C)CC4)CC5=O)c[nH]c3n2)CC1] | 1857 |
| 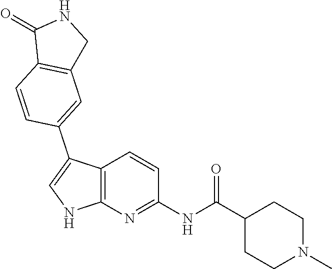 [SMILES: CN1CCC(C(=O)Nc2ccc3c(-c4ccc5c(c4)CNC5=O)c[nH]c3n2)CC1] | 1858 |
| 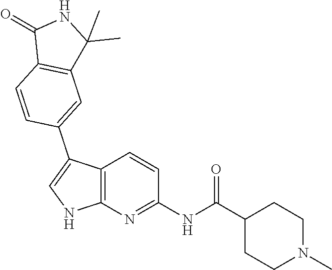 [SMILES: CN1CCC(C(=O)Nc2ccc3c(-c4ccc5c(c4)C(C)(C)NC5=O)c[nH]c3n2)CC1] | 1859 |
| 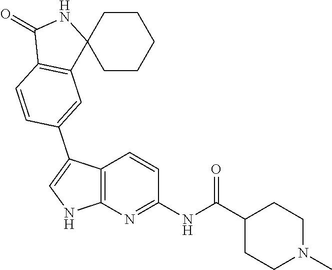 [SMILES: CN1CCC(C(=O)Nc2ccc3c(-c4ccc5c(c4)C4(CCCCC4)NC5=O)c[nH]c3n2)CC1] | 1860 |
| 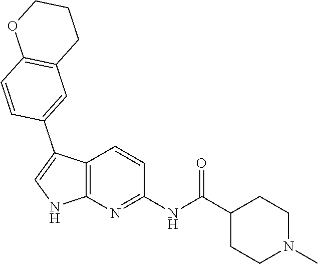 [SMILES: CN1CCC(C(=O)Nc2ccc3c(-c4ccc5c(c4)CCCO5)c[nH]c3n2)CC1] | 1861 |
| 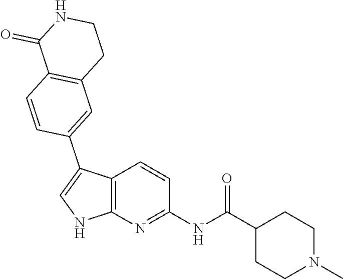 [SMILES: CN1CCC(C(=O)Nc2ccc3c(-c4ccc5c(c4)CCNC5=O)c[nH]c3n2)CC1] | 1862 |
| 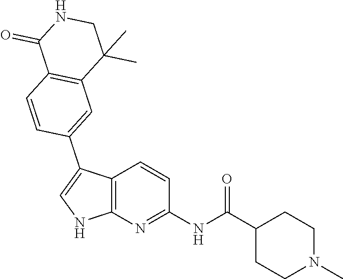 [SMILES: CN1CCC(C(=O)Nc2ccc3c(-c4ccc5c(c4)C(C)(C)CNC5=O)c[nH]c3n2)CC1] | 1863 |
| 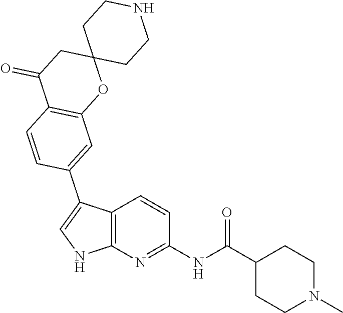 [SMILES: CN1CCC(C(=O)Nc2ccc3c(-c4ccc5c(c4)OC4(CCNCC4)CC5=O)c[nH]c3n2)CC1] | 1864 |
| 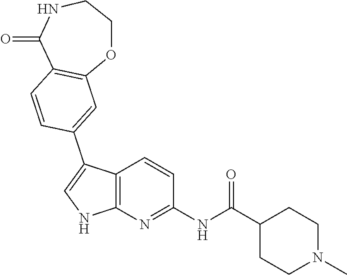 [SMILES: CN1CCC(C(=O)Nc2ccc3c(-c4ccc5c(c4)OCCNC5=O)c[nH]c3n2)CC1] | 1865 |
| 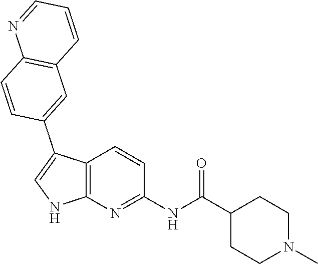 [SMILES: CN1CCC(C(=O)Nc2ccc3c(-c4ccc5ncccc5c4)c[nH]c3n2)CC1] | 1866 |
| 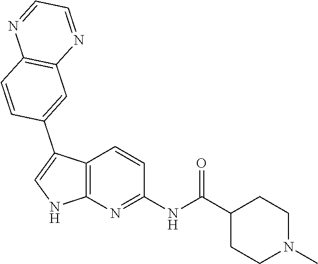 [SMILES: CN1CCC(C(=O)Nc2ccc3c(-c4ccc5nccnc5c4)c[nH]c3n2)CC1] | 1867 |
| 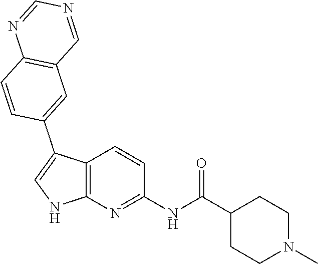 [SMILES: CN1CCC(C(=O)Nc2ccc3c(-c4ccc5ncncc5c4)c[nH]c3n2)CC1] | 1868 |
| 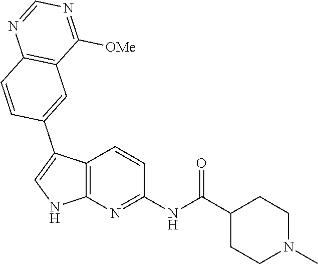 [SMILES: COc1ncnc2ccc(-c3c[nH]c4nc(NC(=O)C5CCN(C)CC5)ccc34)cc12] | 1869 |
| 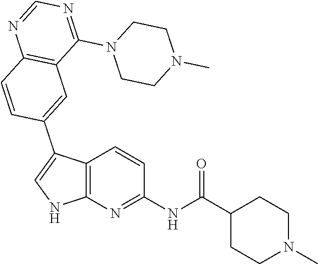 [SMILES: CN1CCC(C(=O)Nc2ccc3c(-c4ccc5ncnc(N6CCN(C)CC6)c5c4)c[nH]c3n2)CC1] | 1870 |
| 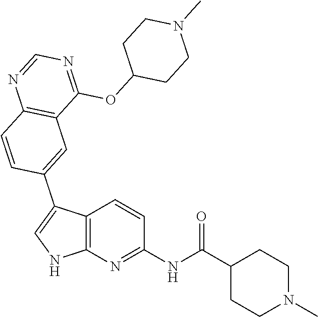 [SMILES: CN1CCC(Oc2ncnc3ccc(-c4c[nH]c5nc(NC(=O)C6CCN(C)CC6)ccc45)cc23)CC1] | 1871 |
| 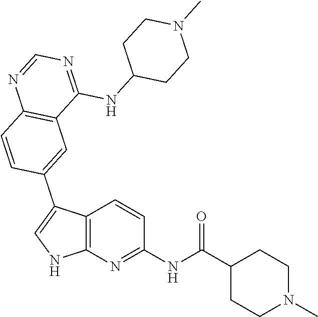 [SMILES: CN1CCC(Nc2ncnc3ccc(-c4c[nH]c5nc(NC(=O)C6CCN(C)CC6)ccc45)cc23)CC1] | 1872 |
| 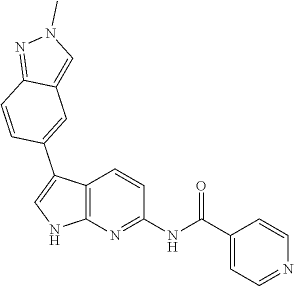 [SMILES: Cn1cc2cc(-c3c[nH]c4nc(NC(=O)c5ccncc5)ccc34)ccc2n1] | 1873 |
| 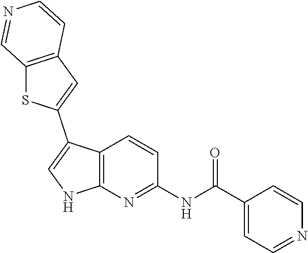 [SMILES: O=C(Nc1ccc2c(-c3cc4ccncc4s3)c[nH]c2n1)c1ccncc1] | 1874 |
| 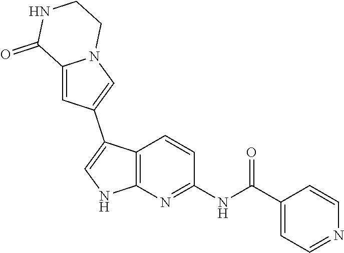 [SMILES: O=C(Nc1ccc2c(-c3cc4n(c3)CCNC4=O)c[nH]c2n1)c1ccncc1] | 1875 |
| 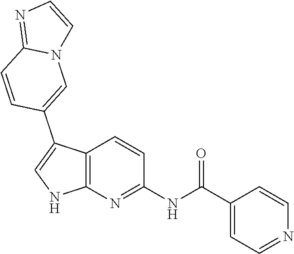 [SMILES: O=C(Nc1ccc2c(-c3ccc4nccn4c3)c[nH]c2n1)c1ccncc1] | 1876 |
| 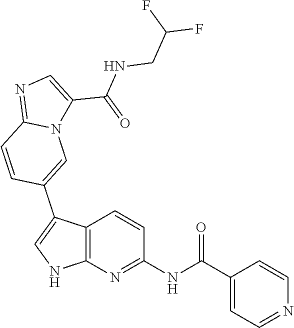 [SMILES: O=C(Nc1ccc2c(-c3ccc4ncc(C(=O)NCC(F)F)n4c3)c[nH]c2n1)c1ccncc1] | 1877 |
| 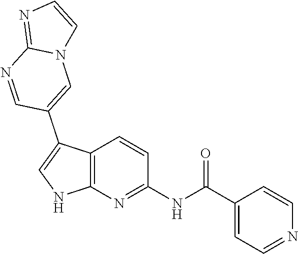 [SMILES: O=C(Nc1ccc2c(-c3cnc4nccn4c3)c[nH]c2n1)c1ccncc1] | 1878 |
| 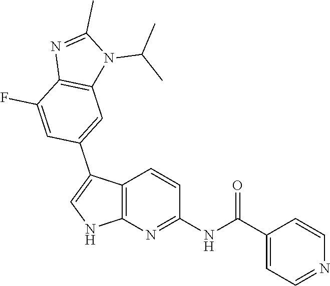 [SMILES: Cc1nc2c(F)cc(-c3c[nH]c4nc(NC(=O)c5ccncc5)ccc34)cc2n1C(C)C] | 1879 |
| 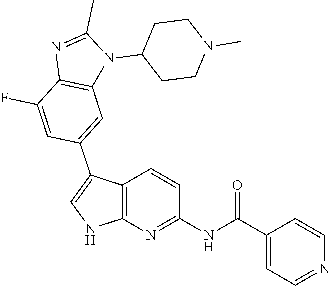 [SMILES: Cc1nc2c(F)cc(-c3c[nH]c4nc(NC(=O)c5ccncc5)ccc34)cc2n1C1CCN(C)CC1] | 1880 |
| 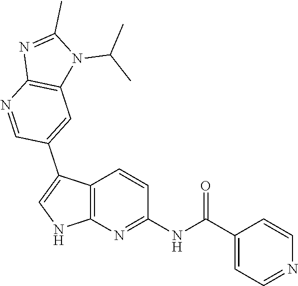 [SMILES: Cc1nc2ncc(-c3c[nH]c4nc(NC(=O)c5ccncc5)ccc34)cc2n1C(C)C] | 1881 |
| 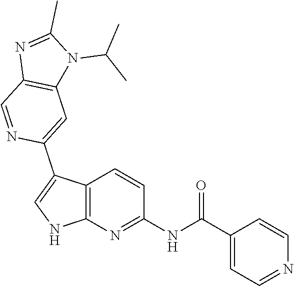 [SMILES: Cc1nc2cnc(-c3c[nH]c4nc(NC(=O)c5ccncc5)ccc34)cc2n1C(C)C] | 1882 |
| 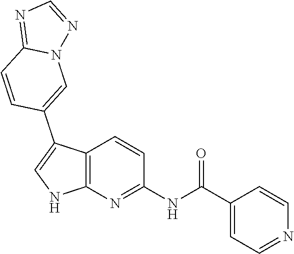 [SMILES: O=C(Nc1ccc2c(-c3ccc4ncnn4c3)c[nH]c2n1)c1ccncc1] | 1883 |
| 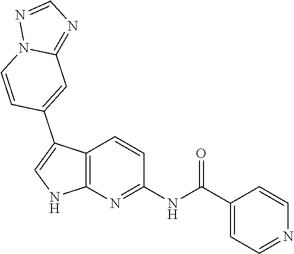 [SMILES: O=C(Nc1ccc2c(-c3ccn4ncnc4c3)c[nH]c2n1)c1ccncc1] | 1884 |
| 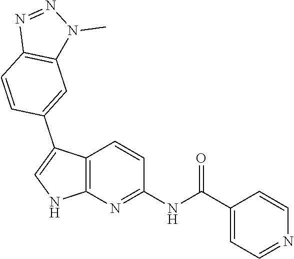 [SMILES: Cn1nnc2ccc(-c3c[nH]c4nc(NC(=O)c5ccncc5)ccc34)cc21] | 1885 |
| 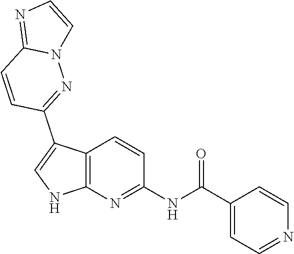 [SMILES: O=C(Nc1ccc2c(-c3ccc4nccn4n3)c[nH]c2n1)c1ccncc1] | 1886 |
| 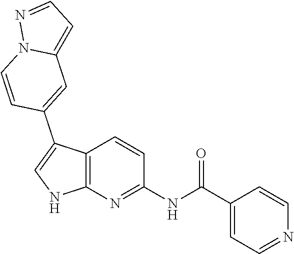 [SMILES: O=C(Nc1ccc2c(-c3ccn4nccc4c3)c[nH]c2n1)c1ccncc1] | 1887 |
| 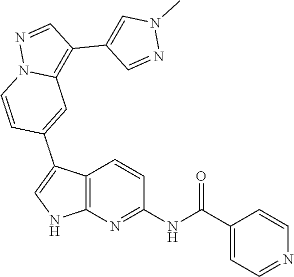 [SMILES: Cn1cc(-c2cnn3ccc(-c4c[nH]c5nc(NC(=O)c6ccncc6)ccc45)cc23)cn1] | 1888 |
| 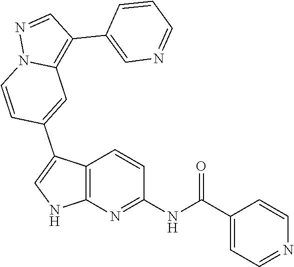 [SMILES: O=C(Nc1ccc2c(-c3ccn4ncc(-c5cccnc5)c4c3)c[nH]c2n1)c1ccncc1] | 1889 |
| 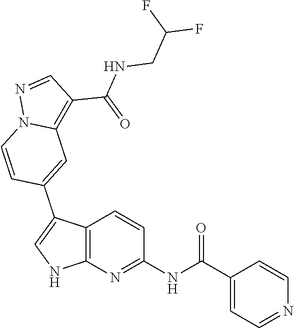 [SMILES: O=C(Nc1ccc2c(-c3ccn4ncc(C(=O)NCC(F)F)c4c3)c[nH]c2n1)c1ccncc1] | 1890 |
| 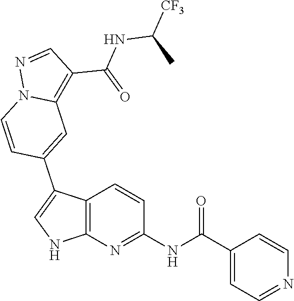 [SMILES: C[C@@H](NC(=O)c1cnn2ccc(-c3c[nH]c4nc(NC(=O)c5ccncc5)ccc34)cc12)C(F)(F)F] | 1891 |
| 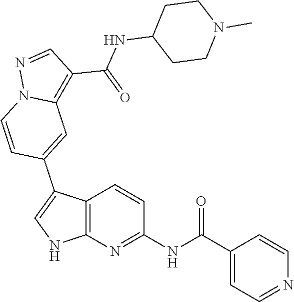 [SMILES: CN1CCC(NC(=O)c2cnn3ccc(-c4c[nH]c5nc(NC(=O)c6ccncc6)ccc45)cc23)CC1] | 1892 |
| 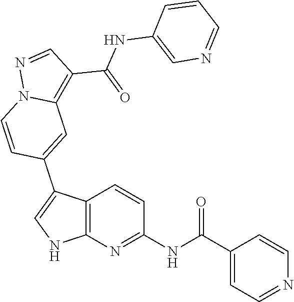 [SMILES: O=C(Nc1ccc2c(-c3ccn4ncc(C(=O)Nc5cccnc5)c4c3)c[nH]c2n1)c1ccncc1] | 1893 |
| 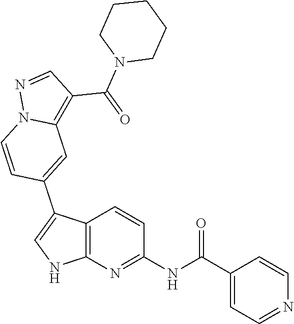 [SMILES: O=C(Nc1ccc2c(-c3ccn4ncc(C(=O)N5CCCCC5)c4c3)c[nH]c2n1)c1ccncc1] | 1894 |
| 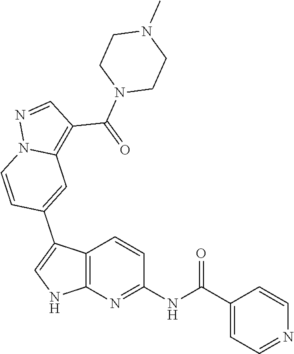 [SMILES: CN1CCN(C(=O)c2cnn3ccc(-c4c[nH]c5nc(NC(=O)c6ccncc6)ccc45)cc23)CC1] | 1895 |
| 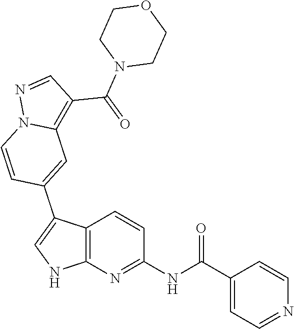 [SMILES: O=C(Nc1ccc2c(-c3ccn4ncc(C(=O)N5CCOCC5)c4c3)c[nH]c2n1)c1ccncc1] | 1896 |
| 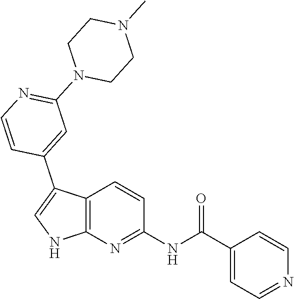 [SMILES: CN1CCN(c2cc(-c3c[nH]c4nc(NC(=O)c5ccncc5)ccc34)ccn2)CC1] | 1897 |
| 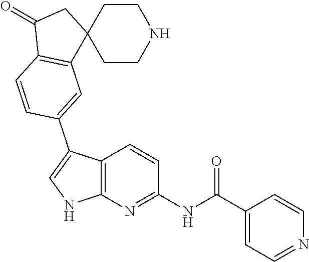 [SMILES: O=C(Nc1ccc2c(-c3ccc4c(c3)C3(CCNCC3)CC4=O)c[nH]c2n1)c1ccncc1] | 1898 |
| 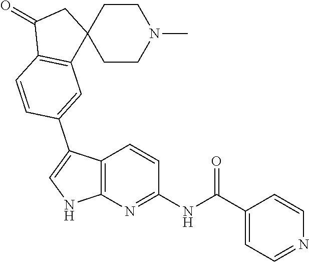 [SMILES: CN1CCC2(CC1)CC(=O)c1ccc(-c3c[nH]c4nc(NC(=O)c5ccncc5)ccc34)cc12] | 1899 |
| 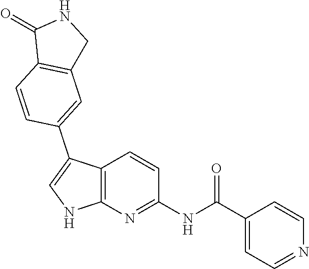 [SMILES: O=C(Nc1ccc2c(-c3ccc4c(c3)CNC4=O)c[nH]c2n1)c1ccncc1] | 1900 |
| 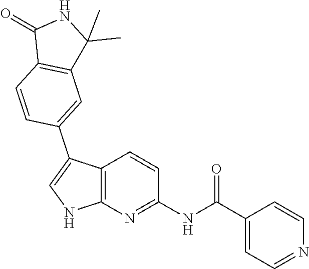 [SMILES: CC1(C)NC(=O)c2ccc(-c3c[nH]c4nc(NC(=O)c5ccncc5)ccc34)cc21] | 1901 |
| 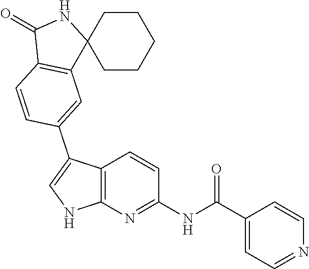 [SMILES: O=C(Nc1ccc2c(-c3ccc4c(c3)C3(CCCCC3)NC4=O)c[nH]c2n1)c1ccncc1] | 1902 |
| 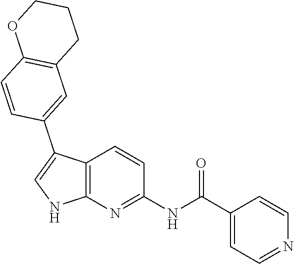 [SMILES: O=C(Nc1ccc2c(-c3ccc4c(c3)CCCO4)c[nH]c2n1)c1ccncc1] | 1903 |
| 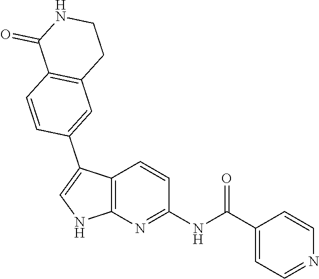 [SMILES: O=C(Nc1ccc2c(-c3ccc4c(c3)CCNC4=O)c[nH]c2n1)c1ccncc1] | 1904 |
| 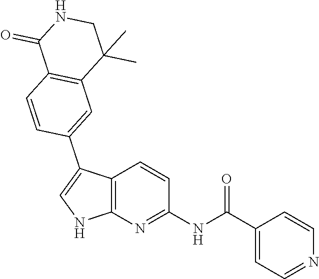 [SMILES: CC1(C)CNC(=O)c2ccc(-c3c[nH]c4nc(NC(=O)c5ccncc5)ccc34)cc21] | 1905 |
| 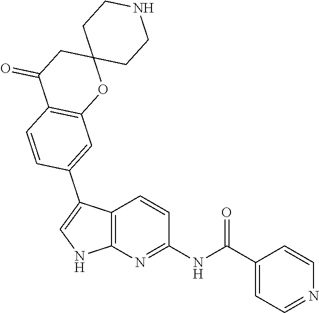 [SMILES: O=C(Nc1ccc2c(-c3ccc4c(c3)OC3(CCNCC3)CC4=O)c[nH]c2n1)c1ccncc1] | 1906 |
| 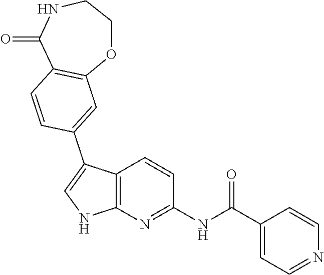 [SMILES: O=C(Nc1ccc2c(-c3ccc4c(c3)OCCNC4=O)c[nH]c2n1)c1ccncc1] | 1907 |
| 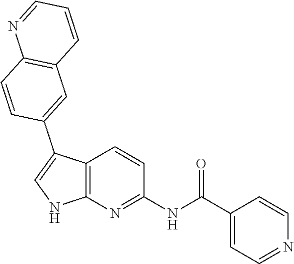 [SMILES: O=C(Nc1ccc2c(-c3ccc4ncccc4c3)c[nH]c2n1)c1ccncc1] | 1908 |
| 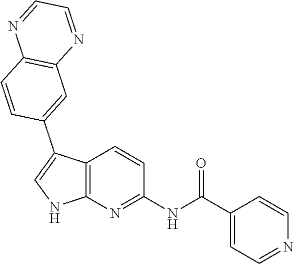 [SMILES: O=C(Nc1ccc2c(-c3ccc4nccnc4c3)c[nH]c2n1)c1ccncc1] | 1909 |
| 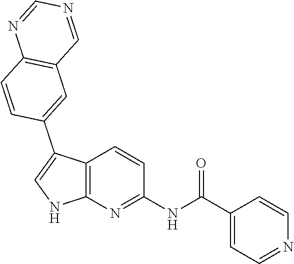 [SMILES: O=C(Nc1ccc2c(-c3ccc4ncncc4c3)c[nH]c2n1)c1ccncc1] | 1910 |
| 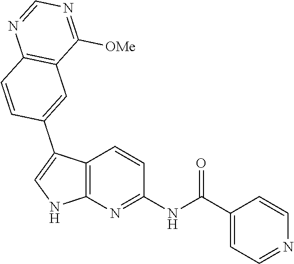 [SMILES: COc1ncnc2ccc(-c3c[nH]c4nc(NC(=O)c5ccncc5)ccc34)cc12] | 1911 |
| 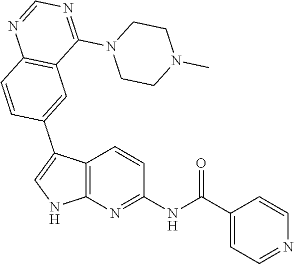 [SMILES: CN1CCN(c2ncnc3ccc(-c4c[nH]c5nc(NC(=O)c6ccncc6)ccc45)cc23)CC1] | 1912 |
| 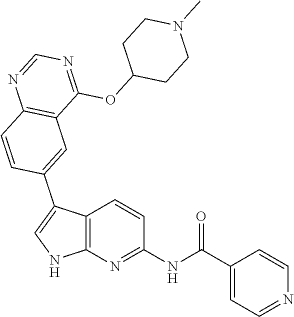 [SMILES: CN1CCC(Oc2ncnc3ccc(-c4c[nH]c5nc(NC(=O)c6ccncc6)ccc45)cc23)CC1] | 1913 |
| 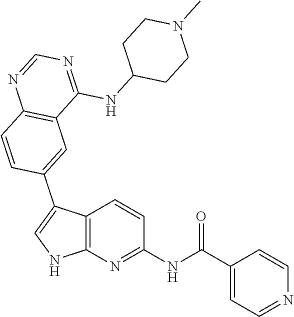 [SMILES: CN1CCC(Nc2ncnc3ccc(-c4c[nH]c5nc(NC(=O)c6ccncc6)ccc45)cc23)CC1] | 1914 |
| 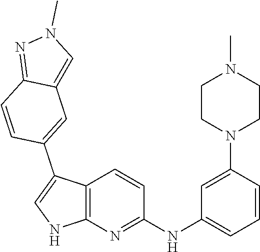 [SMILES: CN1CCN(c2cccc(Nc3ccc4c(-c5ccc6nn(C)cc6c5)c[nH]c4n3)c2)CC1] | 1915 |
| 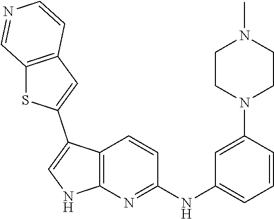 [SMILES: CN1CCN(c2cccc(Nc3ccc4c(-c5cc6ccncc6s5)c[nH]c4n3)c2)CC1] | 1916 |
| 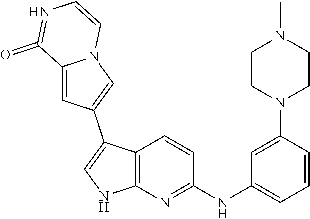 | 1917 |
| 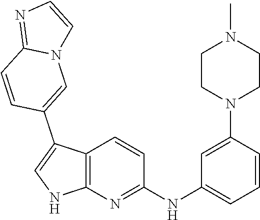 [SMILES: CN1CCN(c2cccc(Nc3ccc4c(-c5ccc6nccn6c5)c[nH]c4n3)c2)CC1] | 1918 |
| 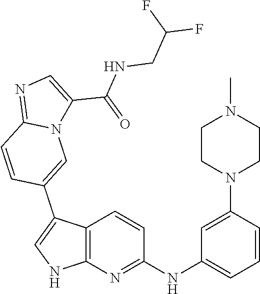 [SMILES: CN1CCN(c2cccc(Nc3ccc4c(-c5ccc6ncc(C(=O)NCC(F)F)n6c5)c[nH]c4n3)c2)CC1] | 1919 |
| 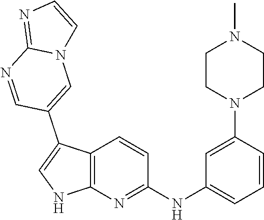 [SMILES: CN1CCN(c2cccc(Nc3ccc4c(-c5cnc6nccn6c5)c[nH]c4n3)c2)CC1] | 1920 |
| 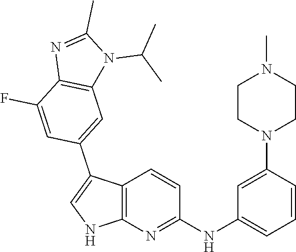 [SMILES: Cc1nc2c(F)cc(-c3c[nH]c4nc(Nc5cccc(N6CCN(C)CC6)c5)ccc34)cc2n1C(C)C] | 1921 |
| 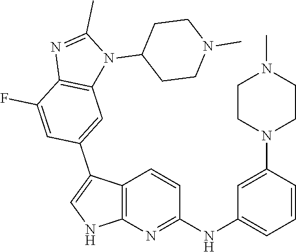 [SMILES: Cc1nc2c(F)cc(-c3c[nH]c4nc(Nc5cccc(N6CCN(C)CC6)c5)ccc34)cc2n1C1CCN(C)CC1] | 1922 |
| 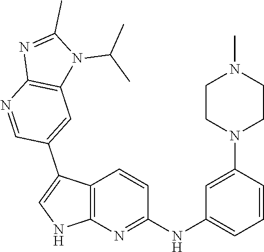 [SMILES: Cc1nc2ncc(-c3c[nH]c4nc(Nc5cccc(N6CCN(C)CC6)c5)ccc34)cc2n1C(C)C] | 1923 |
| 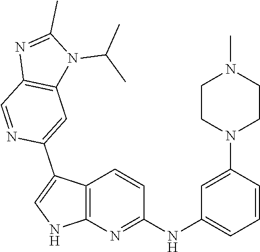 [SMILES: Cc1nc2cnc(-c3c[nH]c4nc(Nc5cccc(N6CCN(C)CC6)c5)ccc34)cc2n1C(C)C] | 1924 |
| 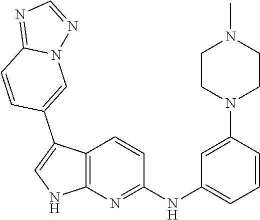 [SMILES: CN1CCN(c2cccc(Nc3ccc4c(-c5ccc6ncnn6c5)c[nH]c4n3)c2)CC1] | 1925 |
| 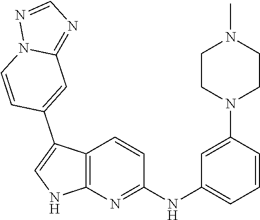 [SMILES: CN1CCN(c2cccc(Nc3ccc4c(-c5ccn6ncnc6c5)c[nH]c4n3)c2)CC1] | 1926 |
| 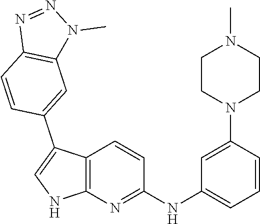 [SMILES: CN1CCN(c2cccc(Nc3ccc4c(-c5ccc6nnn(C)c6c5)c[nH]c4n3)c2)CC1] | 1927 |
| 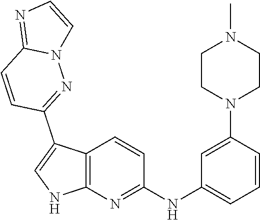 [SMILES: CN1CCN(c2cccc(Nc3ccc4c(-c5ccc6nccn6n5)c[nH]c4n3)c2)CC1] | 1928 |
| 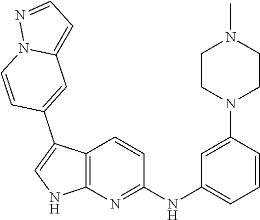 [SMILES: CN1CCN(c2cccc(Nc3ccc4c(-c5ccn6nccc6c5)c[nH]c4n3)c2)CC1] | 1929 |
| 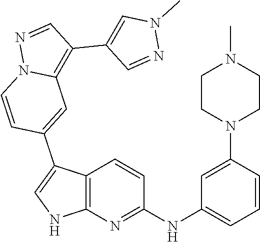 [SMILES: CN1CCN(c2cccc(Nc3ccc4c(-c5ccn6ncc(-c7cnn(C)c7)c6c5)c[nH]c4n3)c2)CC1] | 1930 |
| 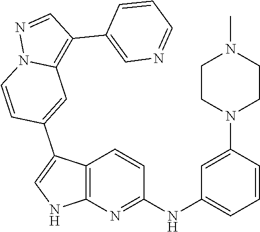 [SMILES: CN1CCN(c2cccc(Nc3ccc4c(-c5ccn6ncc(-c7cccnc7)c6c5)c[nH]c4n3)c2)CC1] | 1931 |
| 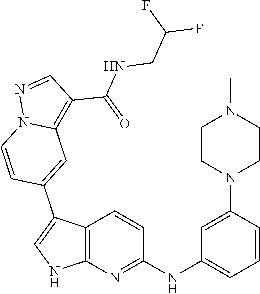 [SMILES: CN1CCN(c2cccc(Nc3ccc4c(-c5ccn6ncc(C(=O)NCC(F)F)c6c5)c[nH]c4n3)c2)CC1] | 1932 |
| 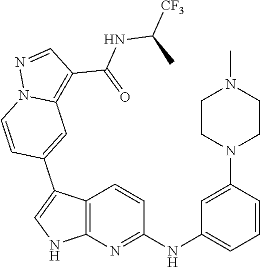 [SMILES: C[C@@H](NC(=O)c1cnn2ccc(-c3c[nH]c4nc(Nc5cccc(N6CCN(C)CC6)c5)ccc34)cc12)C(F)(F)F] | 1933 |
| 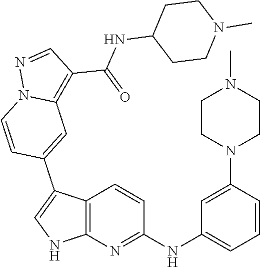 [SMILES: CN1CCC(NC(=O)c2cnn3ccc(-c4c[nH]c5nc(Nc6cccc(N7CCN(C)CC7)c6)ccc45)cc23)CC1] | 1934 |
| 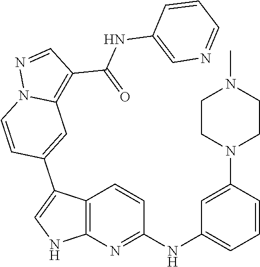 [SMILES: CN1CCN(c2cccc(Nc3ccc4c(-c5ccn6ncc(C(=O)Nc7cccnc7)c6c5)c[nH]c4n3)c2)CC1] | 1935 |
| 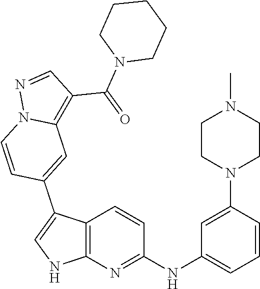 [SMILES: CN1CCN(c2cccc(Nc3ccc4c(-c5ccn6ncc(C(=O)N7CCCCC7)c6c5)c[nH]c4n3)c2)CC1] | 1936 |
| 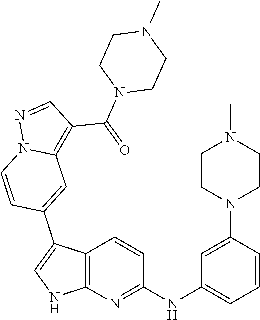 [SMILES: CN1CCN(C(=O)c2cnn3ccc(-c4c[nH]c5nc(Nc6cccc(N7CCN(C)CC7)c6)ccc45)cc23)CC1] | 1937 |
| 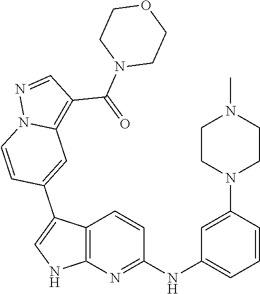 [SMILES: CN1CCN(c2cccc(Nc3ccc4c(-c5ccn6ncc(C(=O)N7CCOCC7)c6c5)c[nH]c4n3)c2)CC1] | 1938 |
| 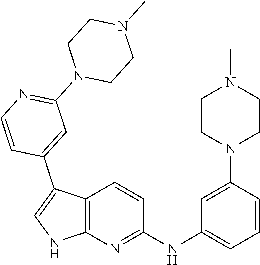 [SMILES: CN1CCN(c2cccc(Nc3ccc4c(-c5ccnc(N6CCN(C)CC6)c5)c[nH]c4n3)c2)CC1] | 1939 |
| 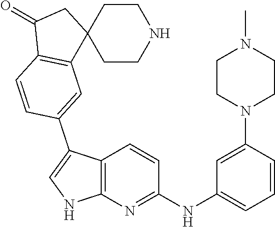 [SMILES: CN1CCN(c2cccc(Nc3ccc4c(-c5ccc6c(c5)C5(CCNCC5)CC6=O)c[nH]c4n3)c2)CC1] | 1940 |
| 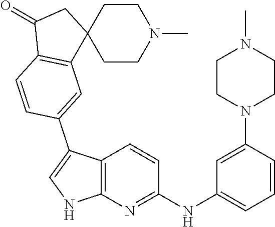 [SMILES: CN1CCN(c2cccc(Nc3ccc4c(-c5ccc6c(c5)C5(CCN(C)CC5)CC6=O)c[nH]c4n3)c2)CC1] | 1941 |
| 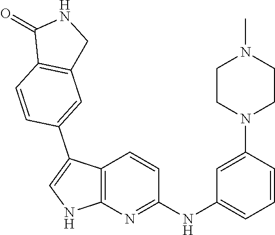 [SMILES: CN1CCN(c2cccc(Nc3ccc4c(-c5ccc6c(c5)CNC6=O)c[nH]c4n3)c2)CC1] | 1942 |
| 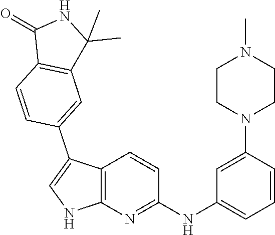 [SMILES: CN1CCN(c2cccc(Nc3ccc4c(-c5ccc6c(c5)C(C)(C)NC6=O)c[nH]c4n3)c2)CC1] | 1943 |
| 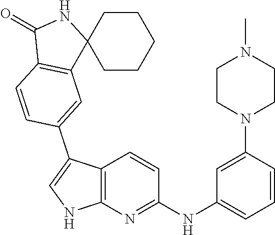 [SMILES: CN1CCN(c2cccc(Nc3ccc4c(-c5ccc6c(c5)C5(CCCCC5)NC6=O)c[nH]c4n3)c2)CC1] | 1944 |
| 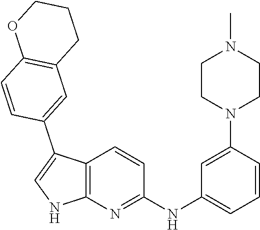 [SMILES: CN1CCN(c2cccc(Nc3ccc4c(-c5ccc6c(c5)CCCO6)c[nH]c4n3)c2)CC1] | 1945 |
| 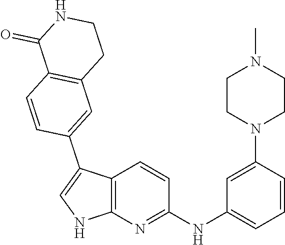 [SMILES: CN1CCN(c2cccc(Nc3ccc4c(-c5ccc6c(c5)CCNC6=O)c[nH]c4n3)c2)CC1] | 1946 |
| 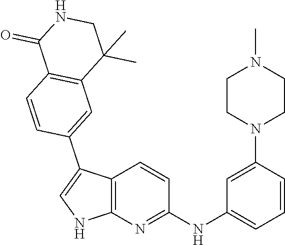 [SMILES: CN1CCN(c2cccc(Nc3ccc4c(-c5ccc6c(c5)C(C)(C)CNC6=O)c[nH]c4n3)c2)CC1] | 1947 |
| 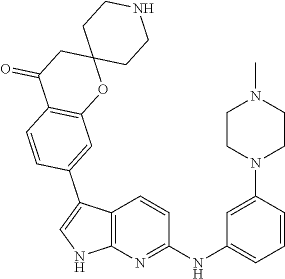 [SMILES: CN1CCN(c2cccc(Nc3ccc4c(-c5ccc6c(c5)OC5(CCNCC5)CC6=O)c[nH]c4n3)c2)CC1] | 1948 |
| 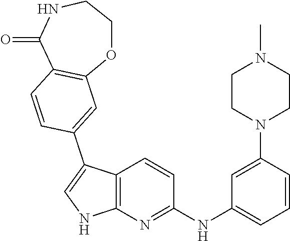 [SMILES: CN1CCN(c2cccc(Nc3ccc4c(-c5ccc6c(c5)OCCNC6=O)c[nH]c4n3)c2)CC1] | 1949 |
| 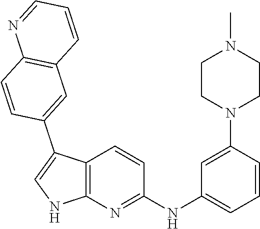 [SMILES: CN1CCN(c2cccc(Nc3ccc4c(-c5ccc6ncccc6c5)c[nH]c4n3)c2)CC1] | 1950 |
| 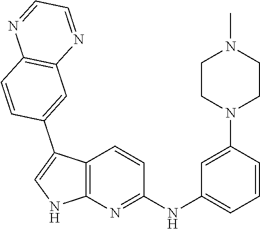 [SMILES: CN1CCN(c2cccc(Nc3ccc4c(-c5ccc6nccnc6c5)c[nH]c4n3)c2)CC1] | 1951 |
| 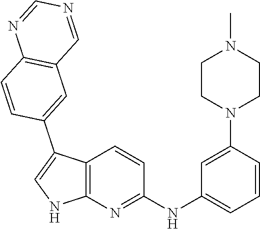 [SMILES: CN1CCN(c2cccc(Nc3ccc4c(-c5ccc6ncncc6c5)c[nH]c4n3)c2)CC1] | 1952 |
| 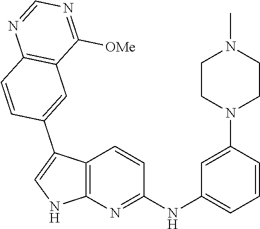 [SMILES: COc1ncnc2ccc(-c3c[nH]c4nc(Nc5cccc(N6CCN(C)CC6)c5)ccc34)cc12] | 1953 |
| 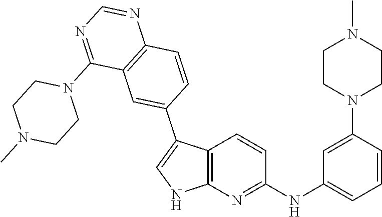 [SMILES: CN1CCN(c2cccc(Nc3ccc4c(-c5ccc6ncnc(N7CCN(C)CC7)c6c5)c[nH]c4n3)c2)CC1] | 1954 |
| 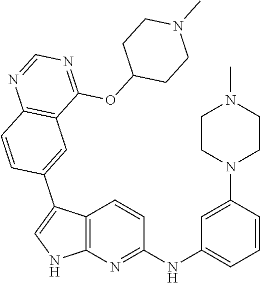 [SMILES: CN1CCC(Oc2ncnc3ccc(-c4c[nH]c5nc(Nc6cccc(N7CCN(C)CC7)c6)ccc45)cc23)CC1] | 1955 |
| 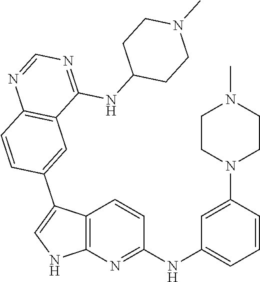 [SMILES: CN1CCC(Nc2ncnc3ccc(-c4c[nH]c5nc(Nc6cccc(N7CCN(C)CC7)c6)ccc45)cc23)CC1] | 1956 |
| 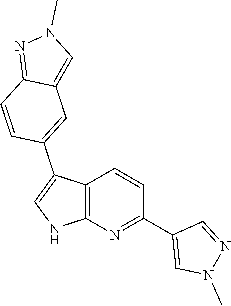 [SMILES: Cn1cc(-c2ccc3c(-c4ccc5nn(C)cc5c4)c[nH]c3n2)cn1] | 1957 |
| 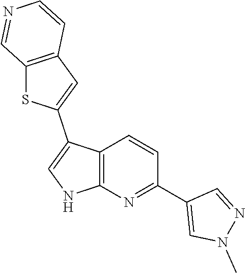 [SMILES: Cn1cc(-c2ccc3c(-c4cc5ccncc5s4)c[nH]c3n2)cn1] | 1958 |
| 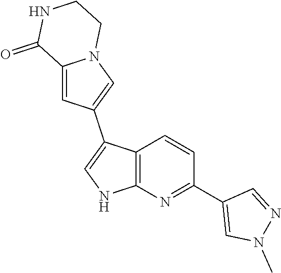 [SMILES: Cn1cc(-c2ccc3c(-c4cc5n(c4)CCNC5=O)c[nH]c3n2)cn1] | 1959 |
| 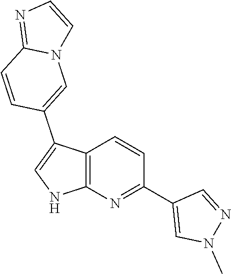 [SMILES: Cn1cc(-c2ccc3c(-c4ccc5nccn5c4)c[nH]c3n2)cn1] | 1960 |
| 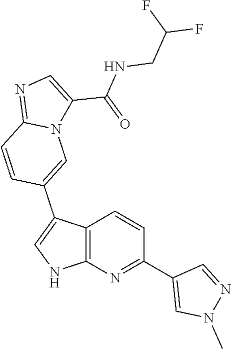 [SMILES: Cn1cc(-c2ccc3c(-c4ccc5ncc(C(=O)NCC(F)F)n5c4)c[nH]c3n2)cn1] | 1961 |
| 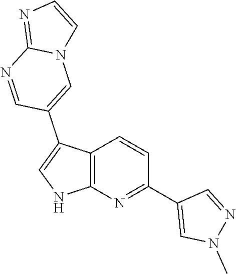 [SMILES: Cn1cc(-c2ccc3c(-c4cnc5nccn5c4)c[nH]c3n2)cn1] | 1962 |
| 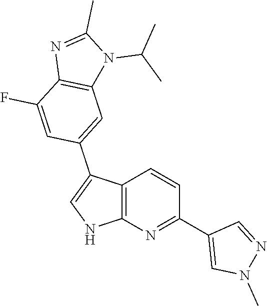 [SMILES: Cc1nc2c(F)cc(-c3c[nH]c4nc(-c5cnn(C)c5)ccc34)cc2n1C(C)C] | 1963 |
| 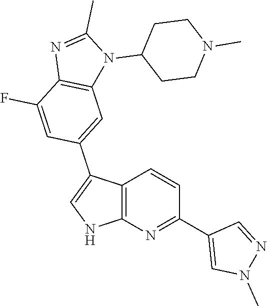 [SMILES: Cc1nc2c(F)cc(-c3c[nH]c4nc(-c5cnn(C)c5)ccc34)cc2n1C1CCN(C)CC1] | 1964 |
| 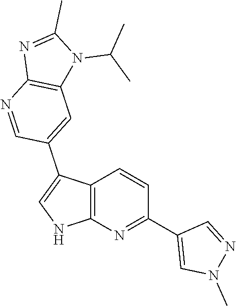 [SMILES: Cc1nc2ncc(-c3c[nH]c4nc(-c5cnn(C)c5)ccc34)cc2n1C(C)C] | 1965 |
| 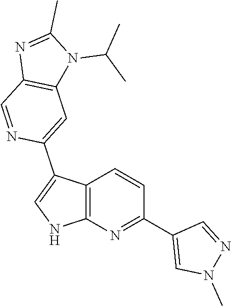 [SMILES: Cc1nc2cnc(-c3c[nH]c4nc(-c5cnn(C)c5)ccc34)cc2n1C(C)C] | 1966 |
| 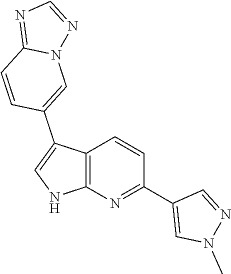 [SMILES: Cn1cc(-c2ccc3c(-c4ccc5ncnn5c4)c[nH]c3n2)cn1] | 1967 |
| 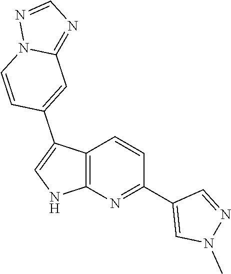 [SMILES: Cn1cc(-c2ccc3c(-c4ccn5ncnc5c4)c[nH]c3n2)cn1] | 1968 |
| 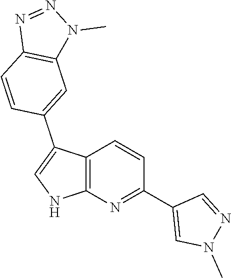 [SMILES: Cn1cc(-c2ccc3c(-c4ccc5nnn(C)c5c4)c[nH]c3n2)cn1] | 1969 |
| 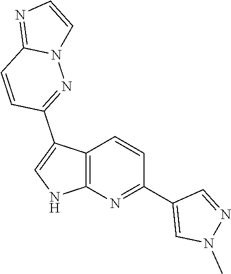 [SMILES: Cn1cc(-c2ccc3c(-c4ccc5nccn5n4)c[nH]c3n2)cn1] | 1970 |
| 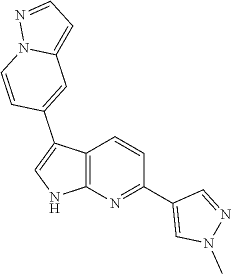 [SMILES: Cn1cc(-c2ccc3c(-c4ccn5nccc5c4)c[nH]c3n2)cn1] | 1971 |
| 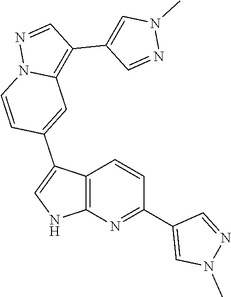 [SMILES: Cn1cc(-c2ccc3c(-c4ccn5ncc(-c6cnn(C)c6)c5c4)c[nH]c3n2)cn1] | 1972 |
| 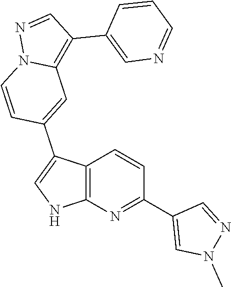 [SMILES: Cn1cc(-c2ccc3c(-c4ccn5ncc(-c6cccnc6)c5c4)c[nH]c3n2)cn1] | 1973 |
| 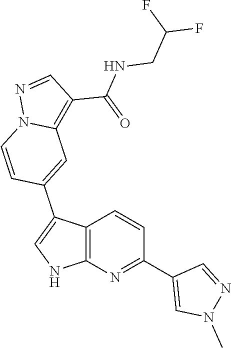 [SMILES: Cn1cc(-c2ccc3c(-c4ccn5ncc(C(=O)NCC(F)F)c5c4)c[nH]c3n2)cn1] | 1974 |
| 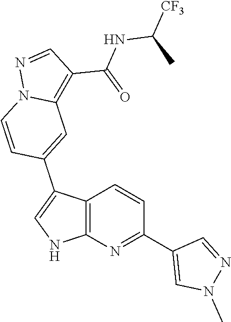 [SMILES: C[C@@H](NC(=O)c1cnn2ccc(-c3c[nH]c4nc(-c5cnn(C)c5)ccc34)cc12)C(F)(F)F] | 1975 |
| 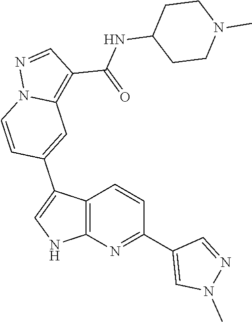 [SMILES: CN1CCC(NC(=O)c2cnn3ccc(-c4c[nH]c5nc(-c6cnn(C)c6)ccc45)cc23)CC1] | 1976 |
| 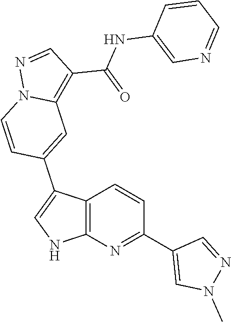 [SMILES: Cn1cc(-c2ccc3c(-c4ccn5ncc(C(=O)Nc6cccnc6)c5c4)c[nH]c3n2)cn1] | 1977 |
| 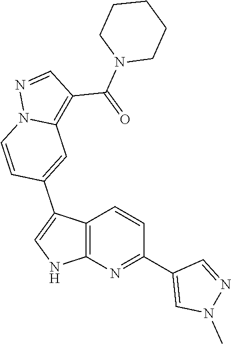 [SMILES: Cn1cc(-c2ccc3c(-c4ccn5ncc(C(=O)N6CCCCC6)c5c4)c[nH]c3n2)cn1] | 1978 |
| 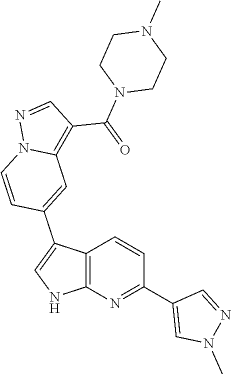 [SMILES: CN1CCN(C(=O)c2cnn3ccc(-c4c[nH]c5nc(-c6cnn(C)c6)ccc45)cc23)CC1] | 1979 |
| 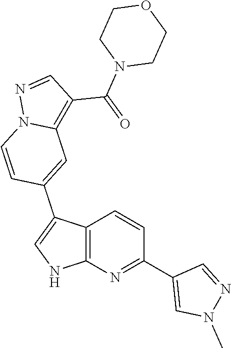 [SMILES: Cn1cc(-c2ccc3c(-c4ccn5ncc(C(=O)N6CCOCC6)c5c4)c[nH]c3n2)cn1] | 1980 |
| 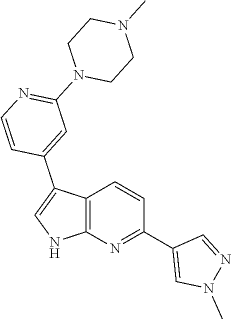 [SMILES: CN1CCN(c2cc(-c3c[nH]c4nc(-c5cnn(C)c5)ccc34)ccn2)CC1] | 1981 |
| 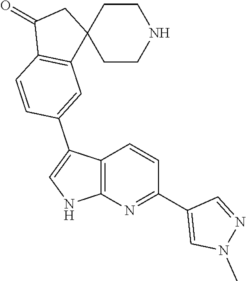 [SMILES: Cn1cc(-c2ccc3c(-c4ccc5c(c4)C4(CCNCC4)CC5=O)c[nH]c3n2)cn1] | 1982 |
| 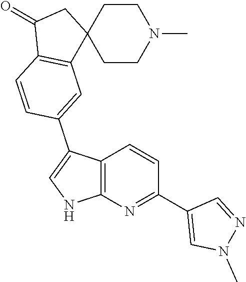 [SMILES: CN1CCC2(CC1)CC(=O)c1ccc(-c3c[nH]c4nc(-c5cnn(C)c5)ccc34)cc12] | 1983 |
| 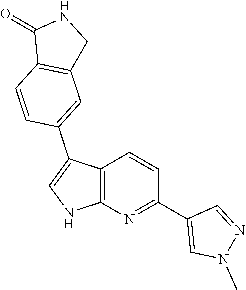 [SMILES: Cn1cc(-c2ccc3c(-c4ccc5c(c4)CNC5=O)c[nH]c3n2)cn1] | 1984 |
| 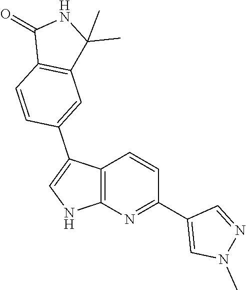 [SMILES: Cn1cc(-c2ccc3c(-c4ccc5c(c4)C(C)(C)NC5=O)c[nH]c3n2)cn1] | 1985 |
| 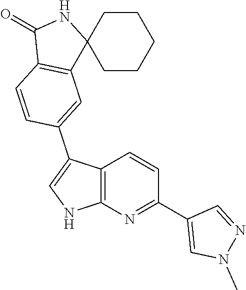 [SMILES: Cn1cc(-c2ccc3c(-c4ccc5c(c4)C4(CCCCC4)NC5=O)c[nH]c3n2)cn1] | 1986 |
| 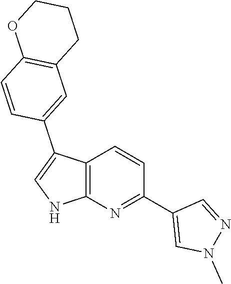 [SMILES: Cn1cc(-c2ccc3c(-c4ccc5c(c4)CCCO5)c[nH]c3n2)cn1] | 1987 |
| 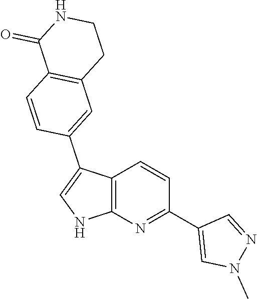 [SMILES: Cn1cc(-c2ccc3c(-c4ccc5c(c4)CCNC5=O)c[nH]c3n2)cn1] | 1988 |
| 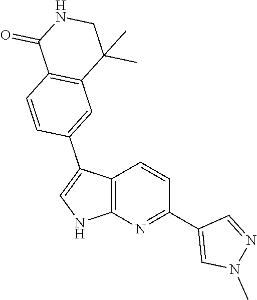 [SMILES: Cn1cc(-c2ccc3c(-c4ccc5c(c4)C(C)(C)CNC5=O)c[nH]c3n2)cn1] | 1989 |
| 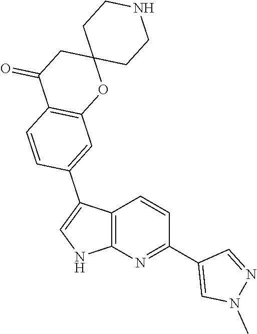 [SMILES: Cn1cc(-c2ccc3c(-c4ccc5c(c4)OC4(CCNCC4)CC5=O)c[nH]c3n2)cn1] | 1990 |
| 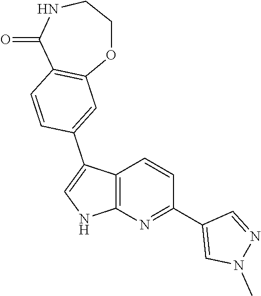 [SMILES: Cn1cc(-c2ccc3c(-c4ccc5c(c4)OCCNC5=O)c[nH]c3n2)cn1] | 1991 |
| 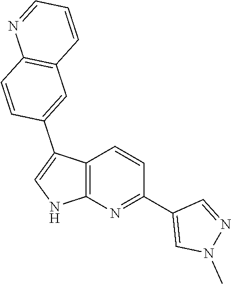 [SMILES: Cn1cc(-c2ccc3c(-c4ccc5ncccc5c4)c[nH]c3n2)cn1] | 1992 |
| 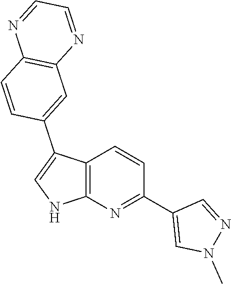 [SMILES: Cn1cc(-c2ccc3c(-c4ccc5nccnc5c4)c[nH]c3n2)cn1] | 1993 |
| 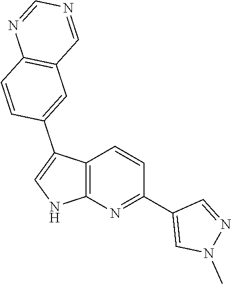 [SMILES: Cn1cc(-c2ccc3c(-c4ccc5ncncc5c4)c[nH]c3n2)cn1] | 1994 |
| 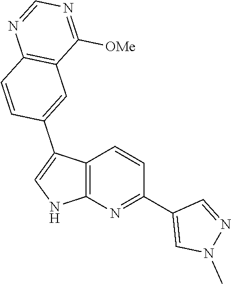 [SMILES: COc1ncnc2ccc(-c3c[nH]c4nc(-c5cnn(C)c5)ccc34)cc12] | 1995 |
| 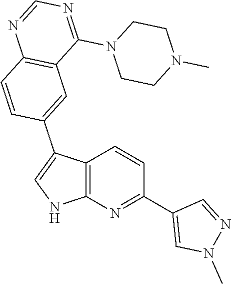 [SMILES: CN1CCN(c2ncnc3ccc(-c4c[nH]c5nc(-c6cnn(C)c6)ccc45)cc23)CC1] | 1996 |
| 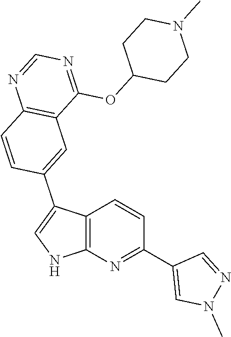 [SMILES: CN1CCC(Oc2ncnc3ccc(-c4c[nH]c5nc(-c6cnn(C)c6)ccc45)cc23)CC1] | 1997 |
| 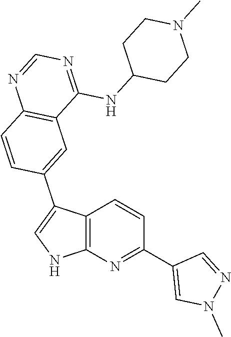 [SMILES: CN1CCC(Nc2ncnc3ccc(-c4c[nH]c5nc(-c6cnn(C)c6)ccc45)cc23)CC1] | 1998 |
| 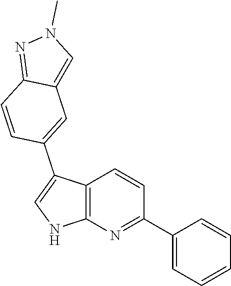 [SMILES: Cn1cc2cc(-c3c[nH]c4nc(-c5ccccc5)ccc34)ccc2n1] | 1999 |
| 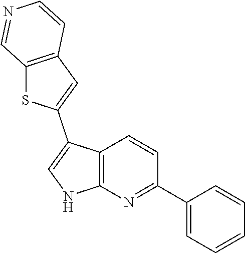 [SMILES: c1ccc(-c2ccc3c(-c4cc5ccncc5s4)c[nH]c3n2)cc1] | 2000 |
| 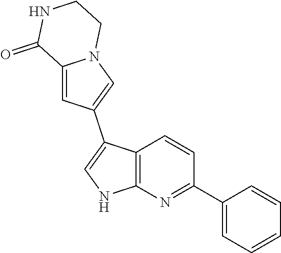 [SMILES: O=C1NCCn2cc(-c3c[nH]c4nc(-c5ccccc5)ccc34)cc21] | 2001 |
| 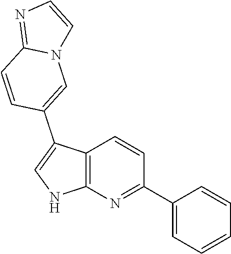 [SMILES: c1ccc(-c2ccc3c(-c4ccc5nccn5c4)c[nH]c3n2)cc1] | 2002 |
| 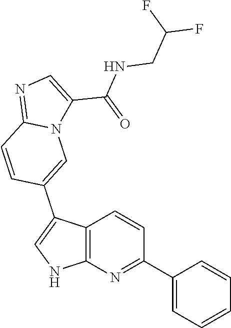 [SMILES: O=C(NCC(F)F)c1cnc2ccc(-c3c[nH]c4nc(-c5ccccc5)ccc34)cn12] | 2003 |
| 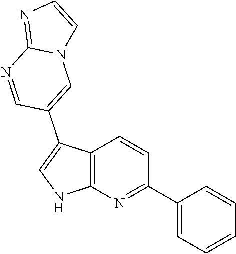 [SMILES: c1ccc(-c2ccc3c(-c4cnc5nccn5c4)c[nH]c3n2)cc1] | 2004 |
| 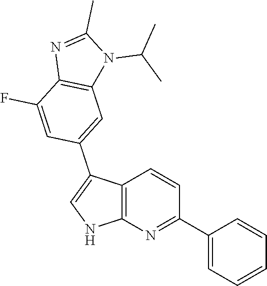 [SMILES: Cc1nc2c(F)cc(-c3c[nH]c4nc(-c5ccccc5)ccc34)cc2n1C(C)C] | 2005 |
| 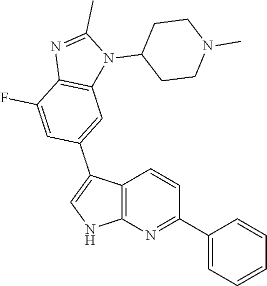 [SMILES: Cc1nc2c(F)cc(-c3c[nH]c4nc(-c5ccccc5)ccc34)cc2n1C1CCN(C)CC1] | 2006 |
| 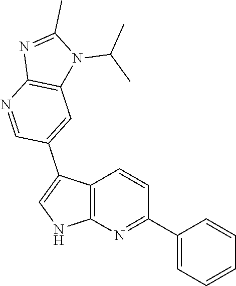 [SMILES: Cc1nc2ncc(-c3c[nH]c4nc(-c5ccccc5)ccc34)cc2n1C(C)C] | 2007 |
| 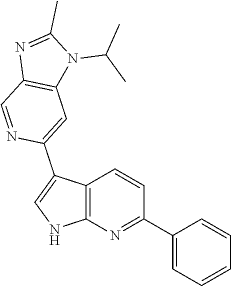 [SMILES: Cc1nc2cnc(-c3c[nH]c4nc(-c5ccccc5)ccc34)cc2n1C(C)C] | 2008 |
| 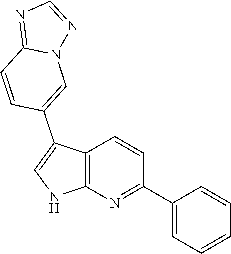 [SMILES: c1ccc(-c2ccc3c(-c4ccc5ncnn5c4)c[nH]c3n2)cc1] | 2009 |
| 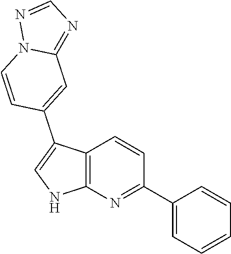 [SMILES: c1ccc(-c2ccc3c(-c4ccn5ncnc5c4)c[nH]c3n2)cc1] | 2010 |
| 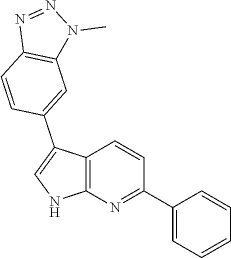 [SMILES: Cn1nnc2ccc(-c3c[nH]c4nc(-c5ccccc5)ccc34)cc21] | 2011 |
| 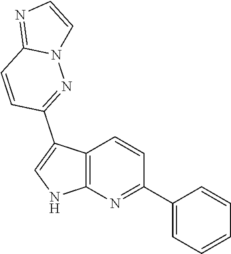 [SMILES: c1ccc(-c2ccc3c(-c4ccc5nccn5n4)c[nH]c3n2)cc1] | 2012 |
| 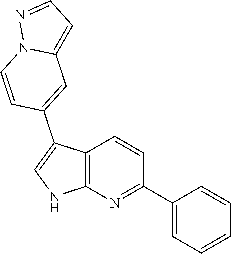 [SMILES: c1ccc(-c2ccc3c(-c4ccn5nccc5c4)c[nH]c3n2)cc1] | 2013 |
| 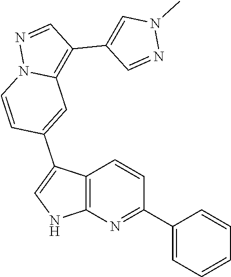 [SMILES: Cn1cc(-c2cnn3ccc(-c4c[nH]c5nc(-c6ccccc6)ccc45)cc23)cn1] | 2014 |
| 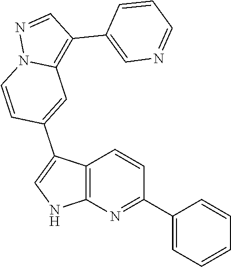 [SMILES: c1ccc(-c2ccc3c(-c4ccn5ncc(-c6cccnc6)c5c4)c[nH]c3n2)cc1] | 2015 |
| 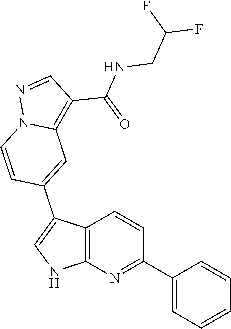 [SMILES: O=C(NCC(F)F)c1cnn2ccc(-c3c[nH]c4nc(-c5ccccc5)ccc34)cc12] | 2016 |
| 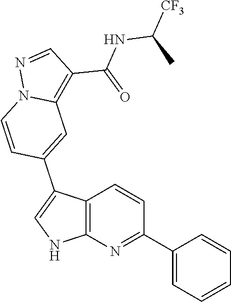 [SMILES: C[C@@H](NC(=O)c1cnn2ccc(-c3c[nH]c4nc(-c5ccccc5)ccc34)cc12)C(F)(F)F] | 2017 |
| 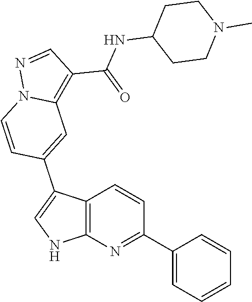 [SMILES: CN1CCC(NC(=O)c2cnn3ccc(-c4c[nH]c5nc(-c6ccccc6)ccc45)cc23)CC1] | 2018 |
| 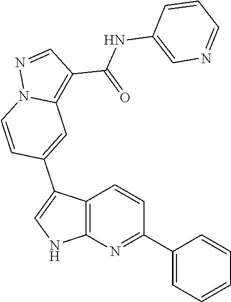 [SMILES: O=C(Nc1cccnc1)c1cnn2ccc(-c3c[nH]c4nc(-c5ccccc5)ccc34)cc12] | 2019 |
| 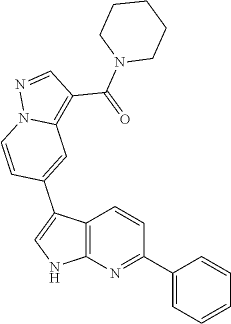 [SMILES: O=C(c1cnn2ccc(-c3c[nH]c4nc(-c5ccccc5)ccc34)cc12)N1CCCCC1] | 2020 |
| 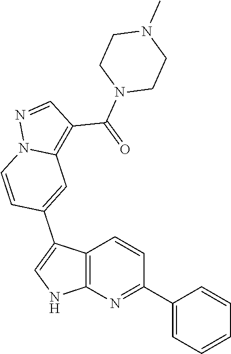 [SMILES: CN1CCN(C(=O)c2cnn3ccc(-c4c[nH]c5nc(-c6ccccc6)ccc45)cc23)CC1] | 2021 |
| 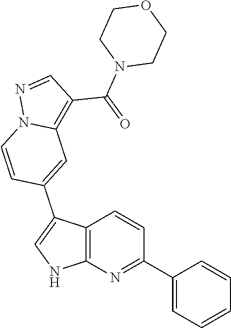 [SMILES: O=C(c1cnn2ccc(-c3c[nH]c4nc(-c5ccccc5)ccc34)cc12)N1CCOCC1] | 2022 |
| 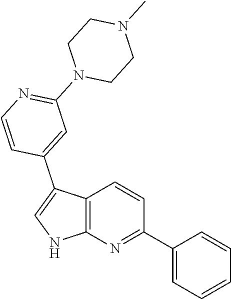 [SMILES: CN1CCN(c2cc(-c3c[nH]c4nc(-c5ccccc5)ccc34)ccn2)CC1] | 2023 |
| 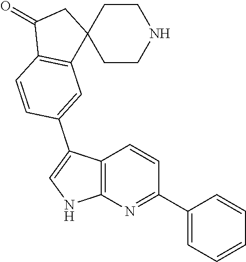 [SMILES: O=C1CC2(CCNCC2)c2cc(-c3c[nH]c4nc(-c5ccccc5)ccc34)ccc21] | 2024 |
| 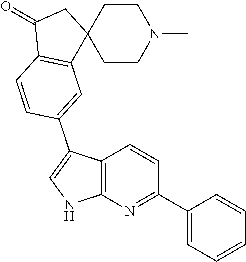 [SMILES: CN1CCC2(CC1)CC(=O)c1ccc(-c3c[nH]c4nc(-c5ccccc5)ccc34)cc12] | 2025 |
| 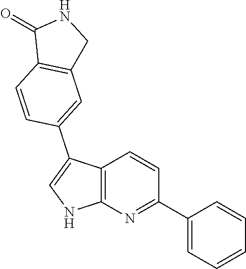 [SMILES: O=C1NCc2cc(-c3c[nH]c4nc(-c5ccccc5)ccc34)ccc21] | 2026 |
| 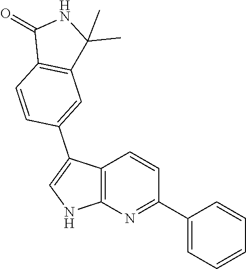 [SMILES: CC1(C)NC(=O)c2ccc(-c3c[nH]c4nc(-c5ccccc5)ccc34)cc21] | 2027 |
| 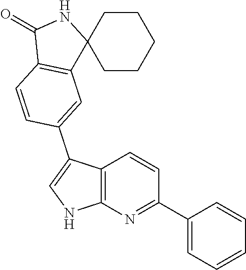 [SMILES: O=C1NC2(CCCCC2)c2cc(-c3c[nH]c4nc(-c5ccccc5)ccc34)ccc21] | 2028 |
| 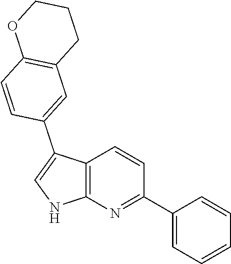 [SMILES: c1ccc(-c2ccc3c(-c4ccc5c(c4)CCCO5)c[nH]c3n2)cc1] | 2029 |
| 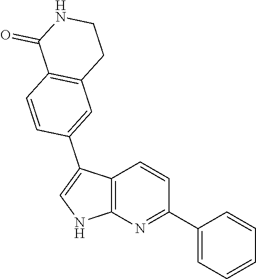 [SMILES: O=C1NCCc2cc(-c3c[nH]c4nc(-c5ccccc5)ccc34)ccc21] | 2030 |
| 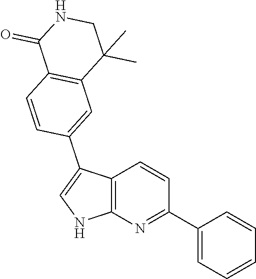 [SMILES: CC1(C)CNC(=O)c2ccc(-c3c[nH]c4nc(-c5ccccc5)ccc34)cc21] | 2031 |
| 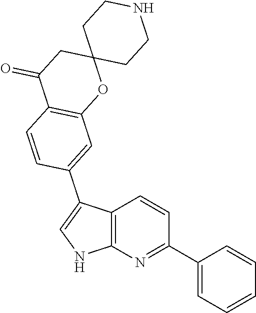 [SMILES: O=C1CC2(CCNCC2)Oc2cc(-c3c[nH]c4nc(-c5ccccc5)ccc34)ccc21] | 2032 |
| 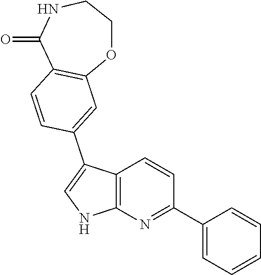 [SMILES: O=C1NCCOc2cc(-c3c[nH]c4nc(-c5ccccc5)ccc34)ccc21] | 2033 |
| 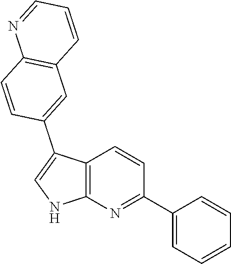 [SMILES: c1ccc(-c2ccc3c(-c4ccc5ncccc5c4)c[nH]c3n2)cc1] | 2034 |
| 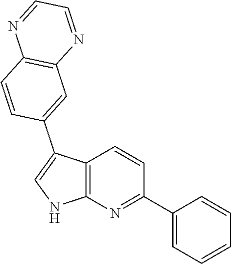 [SMILES: c1ccc(-c2ccc3c(-c4ccc5nccnc5c4)c[nH]c3n2)cc1] | 2035 |
| 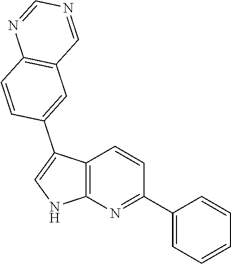 [SMILES: c1ccc(-c2ccc3c(-c4ccc5ncncc5c4)c[nH]c3n2)cc1] | 2036 |
| 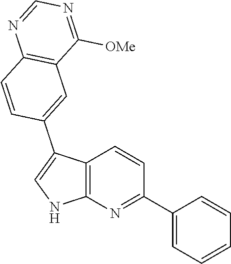 [SMILES: COc1ncnc2ccc(-c3c[nH]c4nc(-c5ccccc5)ccc34)cc12] | 2037 |
| 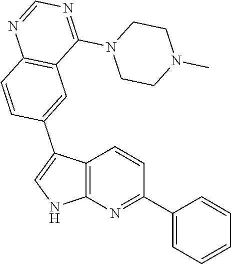 [SMILES: CN1CCN(c2ncnc3ccc(-c4c[nH]c5nc(-c6ccccc6)ccc45)cc23)CC1] | 2038 |
| 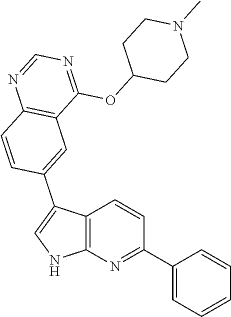 [SMILES: CN1CCC(Oc2ncnc3ccc(-c4c[nH]c5nc(-c6ccccc6)ccc45)cc23)CC1] | 2039 |
| 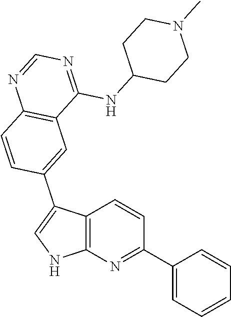 [SMILES: CN1CCC(Nc2ncnc3ccc(-c4c[nH]c5nc(-c6ccccc6)ccc45)cc23)CC1] | 2040 |
| 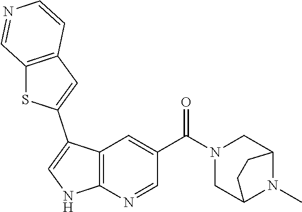 [SMILES: CN1C2CCC1CN(C(=O)c1cnc3[nH]cc(-c4cc5ccncc5s4)c3c1)C2] | 2041 |
| 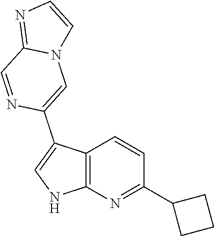 [SMILES: c1cn2cc(-c3c[nH]c4nc(C5CCC5)ccc34)ncc2n1] | 2042 |
| 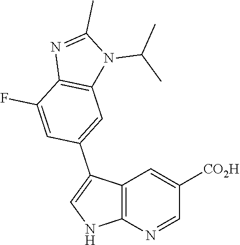 [SMILES: Cc1nc2c(F)cc(-c3c[nH]c4ncc(C(=O)O)cc34)cc2n1C(C)C] | 2043 |
| 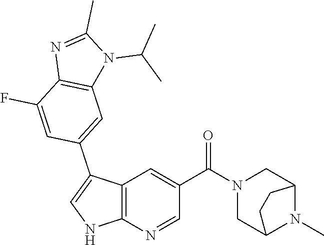 [SMILES: Cc1nc2c(F)cc(-c3c[nH]c4ncc(C(=O)N5CC6CCC(C5)N6C)cc34)cc2n1C(C)C] | 2044 |
| 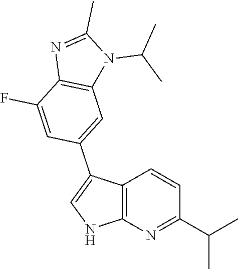 [SMILES: Cc1nc2c(F)cc(-c3c[nH]c4nc(C(C)C)ccc34)cc2n1C(C)C] | 2045 |
| 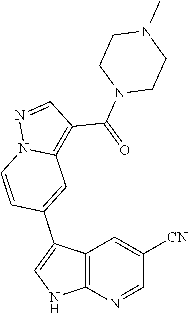 [SMILES: CN1CCN(C(=O)c2cnn3ccc(-c4c[nH]c5ncc(C#N)cc45)cc23)CC1] | 2046 |
| 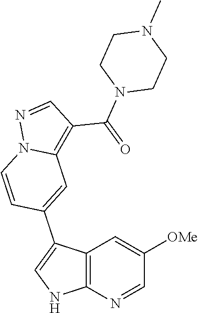 [SMILES: COc1cnc2[nH]cc(-c3ccn4ncc(C(=O)N5CCN(C)CC5)c4c3)c2c1] | 2047 |
| 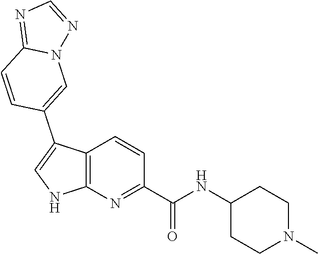 [SMILES: CN1CCC(NC(=O)c2ccc3c(-c4ccc5ncnn5c4)c[nH]c3n2)CC1] | 2048 |
| 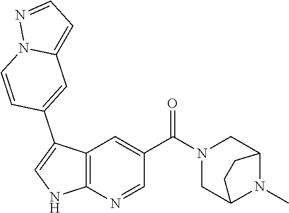 [SMILES: CN1C2CCC1CN(C(=O)c1cnc3[nH]cc(-c4ccn5nccc5c4)c3c1)C2] | 2049 |
| 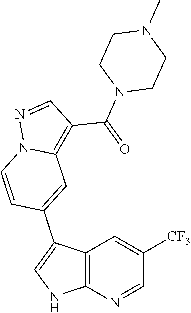 [SMILES: CN1CCN(C(=O)c2cnn3ccc(-c4c[nH]c5ncc(C(F)(F)F)cc45)cc23)CC1] | 2050 |
| 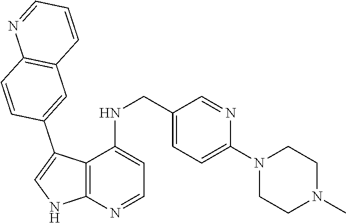 [SMILES: CN1CCN(c2ccc(CNc3ccnc4[nH]cc(-c5ccc6ncccc6c5)c34)cn2)CC1] | 2051 |
| 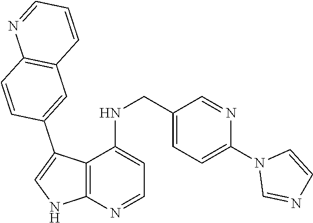 [SMILES: c1cnc2ccc(-c3c[nH]c4nccc(NCc5ccc(-n6ccnc6)nc5)c34)cc2c1] | 2052 |
| 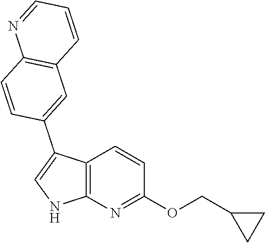 [SMILES: c1cnc2ccc(-c3c[nH]c4nc(OCC5CC5)ccc34)cc2c1] | 2053 |
| 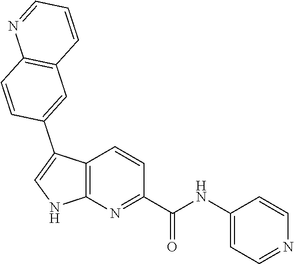 [SMILES: O=C(Nc1ccncc1)c1ccc2c(-c3ccc4ncccc4c3)c[nH]c2n1] | 2054 |
| 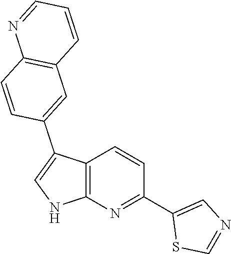 [SMILES: c1cnc2ccc(-c3c[nH]c4nc(-c5cncs5)ccc34)cc2c1] | 2055 |
| 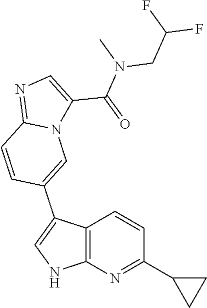 [SMILES: CN(CC(F)F)C(=O)c1cnc2ccc(-c3c[nH]c4nc(C5CC5)ccc34)cn12] | 2056 |

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

The compounds provided herein may also be useful in combination (administered together or sequentially) with other known agents.

Non-limiting examples of diseases which can be treated with a combination of a compound of Formulas I, Ia, Ib, Ic, Id, and Ie, and another active agent are colorectal cancer, ovarian cancer, hepatocellular carcinoma, head and neck squamous cell carcinoma, acute lymphoblastic leukemia (ALL), pancreatic cancer, brain tumors, acute megakaryoblastic leukemia (AMKL), and osteoarthritis. For example, a compound of Formulas I, Ia, Ib, Ic, Id, and Ie can be combined with one or more chemotherapeutic compounds.

In some embodiments, hepatocellular carcinoma can be treated with a combination of a compound of Formulas I, Ia, Ib, Ic, Id, and Ie, and one or more of the following drugs/therapies: sorafenib (Nexavar®); regorafenib (Stivarga®, Regonix®), nivolumab (Opdivo®); lenvatinib (Lenvima®); Pembrolizumab (Keytruda®); cabozantinib (Cometriq®, Cabometyx®); 5-fluorouracil (5-FU®); ramucirumab (Cyramza®); combination of gemcitabine and oxaliplatin (GEMOX). Other therapies that can be performed in combination with a compound of Formulas I, Ia, Ib, Ic, Id, and Ie are i) transcatheter arterial chemoembolization (TACE) in combination with doxorubicin (DOXIL®), cisplatin, or mitomycin C (Mitosol®, Mutamycin®, Jelmyto®); ii) low-dose brachytherapy.

In some embodiments, head and neck squamous cell carcinoma can be treated with a combination of a compound of Formulas I, Ia, Ib, Ic, Id, and Ie, and one or more of the following drugs/therapies: TransOral Robotic Surgery (TORS); TORS with radiation therapy; larotrectinib (Vitrakvi®); EGFR inhibitors, e.g., erlotinib (Tarceva®), osimertinib (Tagrisso®), neratinib (Nerlynx®), gefitinib (Iressa®), cetuximab (Erbitux®), panitumumab (Vectibix®), dacomitinib (Vizimpro®), lapatinib (Tykerb®), necitumumab (Portrazza), and vandetanib (Caprelsa®).

In some embodiments, acute lymphoblastic leukemia (ALL) can be treated with a combination of a compound of Formulas I, Ia, Ib, Ic, Id, and Ie, and one or more of the following drugs/therapies: remission induction therapy; consolidation therapy; nelarabine (Arranon®); Asparaginase Erwinia Chrysanthemi (Erwinaze®); Asparaginase Erwinia Chrysanthemi (Recombinant)-rywn (Rylaze®); calaspargase Pegol-mknl (Asparlas®); inotuzumab ozogamicin (Besponsa®); blinatumomab (Blincyto®); daunorubicin hydrochloride (Cerubidine®); clofarabine (Clolar®); cyclophosphamide; methotrexate sodium (Trexall®); cytarabine (Cytosar-U®); dasatinib (Sprycel®); dexamethasone; imatinib mesylate (Gleevec®); ponatinib hydrochloride (Iclusig®); mercaptopurine (Purinethol®, Purixan®); tisagenlecleucel (Kymriah®); vincristine sulfate liposome (Marqibo®); pegaspargase (Oncaspar®); prednisone; daunorubicin hydrochloride (Rubidomycin®); and vincristine sulfate.

In some embodiments, pancreatic cancer can be treated with a combination of a compound of Formulas I, Ia, Ib, Ic, Id, and Ie, and one or more of the following drugs/therapies: ablation and embolization treatment; gemcitabine (Gemzar®); 5-fluorouracil (5-FU®); oxaliplatin (Eloxatin®); albumin-bound paclitaxel (Abraxane®); capecitabine (Xeloda®); cisplatin; irinotecan (Camptosar®); liposomal Irinotecan (Onivyde®); paclitaxel (Taxol®), and docetaxel (Taxotere®).

In some embodiments, brain tumors can be treated with a combination of a compound of Formulas I, Ia, Ib, Ic, Id, and Ie, and one or more of the following drugs/therapies: carmustine can be administered by way of a gliadel wafer; for glioblastoma and high-grade glioma, radiation therapy with daily low-dose temozolomide (Temodar®) followed by monthly doses of temozolomide after radiation therapy for 6 months to 1 year; lomustine (Gleostine®), procarbazine (Matulane®), and vincristine (Vincasar®), have been used along with radiation therapy; anti-angiogenesis therapy with bevacizumab (Avastin®, Mvasi®); and targeted therapy using larotrectinib (Vitrakvi®).

In some embodiments, acute megakaryoblastic leukemia (AMKL) can be treated with a combination of a compound of Formulas I, Ia, Ib, Ic, Id, and Ie, and one or more of the following drugs/therapies: cytarabine (Cytosar-U®), etoposide (Vepesid®), and anthracycline drugs. Anthracyclines include daunorubicin (Cerubidine®), idarubicin (Idamycin®), and mitoxantrone (Novantrone®).

In some embodiments, acute myeloid leukemia (AML) can be treated with a combination of a compound of Formulas I, Ia, Ib, Ic, Id, and Ie, and one or more of the following drugs/therapies: venetoclax and hypomethylating agents (e.g., decitabine, azacitidine), induction chemotherapy (cytarabine and an anthracycline (e.g., daunorubicin or idarubicin), all-trans-retinoic acid (ATRA) and either arsenic trioxide (ATO) monotherapy or an anthracycline), consolidation therapy (cytarabine).

In some embodiments, myelodysplastic syndrome (MDS) can be treated with a combination of a compound of Formulas I, Ia, Ib, Ic, Id, and Ie, and one or more of the following drugs/therapies: 5-azacytidine, decitabine, lenalidomide, and decitabine/cedazuridine (Inqovi®).

In some embodiments, colorectal cancer can be treated with a combination of a compound of Formulas I, Ia, Ib, Ic, Id, and Ie, and one or more of the following drugs: 5-Fluorouracil (5-FU), which can be administered with the vitamin-like drug leucovorin (also called folinic acid); capecitabine (XELODA®), irinotecan (CAMPOSTAR®), oxaliplatin (ELOXATIN®). Examples of combinations of these drugs which could be further combined with a compound of Formulas I, Ia, Ib, Ic, Id, and Ie are FOLFOX (5-FU, leucovorin, and oxaliplatin), FOLFIRI (5-FU, leucovorin, and irinotecan), FOLFOXIRI (leucovorin, 5-FU, oxaliplatin, and irinotecan) and CapeOx (Capecitabine and oxaliplatin). For rectal cancer, chemo with 5-FU or capecitabine combined with radiation may be given before surgery (neoadjuvant treatment).

In some embodiments, ovarian cancer can be treated with a combination of a compound of Formulas I, Ia, Ib, Ic, Id, and Ie, and one or more of the following drugs: Topotecan, Liposomal doxorubicin (DOXIL®), Gemcitabine (GEMZAR®), Cyclophosphamide (CYTOXAN®), Vinorelbine (NAVELBINE®), Ifosfamide (IFEX®), Etoposide (VP-16), Altretamine (HEXALEN®), Capecitabine (XELODA®), Irinotecan (CPT-11, CAMPTOSAR®), Melphalan, Pemetrexed (ALIMTA®) and Albumin bound paclitaxel (nab-paclitaxel, ABRAXANE®). Examples of combinations of these drugs which could be further combined with a compound of Formulas I, Ia, Ib, Ic, Id, and Ie are TIP (paclitaxel [Taxol], ifosfamide, and cisplatin), VeIP (vinblastine, ifosfamide, and cisplatin) and VIP (etoposide [VP-16], ifosfamide, and cisplatin). Ovarian cancer can also be treated with a combination of a compound of Formula (I) and immune checkpoint blockade (ICB) therapy.

In some embodiments, a compound of Formulas I, Ia, Ib, Ic, Id, and Ie can be used to treat cancer in combination with any of the following methods: (a) Hormone therapy such as aromatase inhibitors, LHRH [luteinizing hormone-releasing hormone] analogs and inhibitors, and others; (b) Ablation or embolization procedures such as radiofrequency ablation (RFA), ethanol (alcohol) ablation, microwave thermotherapy and cryosurgery (cryotherapy); (c) Chemotherapy using alkylating agents such as cisplatin and carboplatin, oxaliplatin, mechlorethamine, cyclophosphamide, chlorambucil and ifosfamide; (d) Chemotherapy using anti-metabolites such as azathioprine and mercaptopurine; (e) Chemotherapy using plant alkaloids and terpenoids such as vinca alkaloids (i.e. Vincristine, Vinblastine, Vinorelbine and Vindesine) and taxanes; (f) Chemotherapy using podophyllotoxin, etoposide, teniposide and docetaxel; (g) Chemotherapy using topoisomerase inhibitors such as irinotecan, topotecan, amsacrine, etoposide, etoposide phosphate, and teniposide; (h) Chemotherapy using cytotoxic antibiotics such as actinomycin, anthracyclines, doxorubicin, daunorubicin, valrubicin, idarubicin, epirubicin, bleomycin, plicamycin and mitomycin; (i) Chemotherapy using tyrosine-kinase inhibitors such as Imatinib mesylate (GLEEVEC®, also known as STI-571), Gefitinib (Iressa, also known as ZD1839), Erlotinib (marketed as TARCEVA®), Bortezomib (VELCADE®), tamoxifen, tofacitinib, crizotinib, Bcl-2 inhibitors (e.g. obatoclax, navitoclax (ABT-263), oblimersen (G3139), venetoclax (ABT-199), Gossypol), PARP inhibitors (e.g. Iniparib, Olaparib, Rucaparib, Niraparib, Talazoparib), PI3K inhibitors (e.g. perifosine in a phase III trial), VEGF Receptor 2 inhibitors (e.g. Apatinib), AN-152, (AEZS-108), Braf inhibitors (e.g. vemurafenib, dabrafenib and LGX818), MEK inhibitors (e.g. trametinib and MEK162), CDK inhibitors, (e.g. PD-0332991), salinomycin and Sorafenib; (j) Chemotherapy using monoclonal antibodies such as Rituximab (marketed as MABTHERA® or RITUXAN®), Trastuzumab (Herceptin also known as ErbB2), Cetuximab (marketed as ERBITUX®), and Bevacizumab (marketed as AVASTIN®); (k) Chemotherapy using KRAS G12C inhibitors such as sotorasib (Lumakras® and Lumykras®), adagrasib (MRTX849), and ARS-3248 (Wellspring Biosciences); (l) Chemotherapy using checkpoint inhibitor therapy such as Ipilimumab (Yervoy®), Nivolumab (Opdivo®), Pembrolizumab (Keytruda), Atezolizumab (Tecentriq®), Avelumab (Bavencio), Durvalumab (Imfinzi), Cemiplimab (Libtayo®), and Spartalizumab (PDR001); (m) Chemotherapy using antibody-drug conjugates (ADC) such as Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, and Loncastuximab tesirine; (n) Chemotherapy using proteasome inhibitors such as carfilzomib, lactacystin, disulfiram, salinosporamide A (marizomib), oprozomib, delanzomib, epoxomicin, MG132, β-hydroxy β-methylbutyric acid (HMB), bortezomib, ixazomib (alone or in in combination with lenalidomide and dexamethasone); and (o) radiation therapy.

In some embodiments, a compound of Formulas I, Ia, Ib, Ic, Id, and Ie, can be used to treat diabetes mellitus in combination with any of the following methods: (a) injections of insulin; (b) biguanides such as metformin (Glucophage), phenformin (DBI), and buformin; (c) thiazolidinediones (TZDs) such as rosiglitazone (Avandia), pioglitazone (Actos), and yroglitazone (Rezulin); (d) lyn kinase activators such as glimepiride (Amaryl®) and tolimidone (MLR-1023); (e) secretagogues such as sulfonylureas (non-limiting examples are acetohexamide, carbutamide, chlorpropamide, glycyclamide (tolcyclamide), metahexamide, tolazamide, tolbutamide, glibenclamide (glyburide), glibornuride, gliclazide, glipizide, gliquidone, glisoxepide, glyclopyramide, and glimepiride) and meglitinides (nonlimiting examples are repaglinide (Prandin), nateglinide (Starlix), and mitiglinide (Glufast)); (f) alpha-glucosidase inhibitors such as acarbose (Glucobay, Precose, Prandase), miglitol (Glyset), and voglibose; (g) injectable incretin mimetics such as glucagon-like peptide-1 (GLP-1) and gastric inhibitory peptide (glucose-dependent insulinotropic peptide, GIP), nonlimiting examples of injectable glucagon-like peptide (GLP) analogs and agonists are exenatide (Exendin-4, marketed as Byetta), liraglutide (Victoza, Saxenda), taspoglutide, lixisenatide (Lyxumia), Semaglutide (Ozempic, Rybelsus), dulaglutide (Trulicity), albiglutide (Tanzeum), nonlimiting examples of dipeptidyl peptidase-4 (DPP-4) inhibitors are sitagliptin (Januvia), vildagliptin (Galvus), saxagliptin (Onglyza), linagliptin (Tradjenta), gemigliptin (Zemiglo), anagliptin (Suiny), teneligliptin (Tenelia), alogliptin (Nesina, Vipidia, Kazano, Vipidomet (with metformin), Oseni, Incresync (with pioglitazone)), trelagliptin (Zafatek, Wedica), omarigliptin (MK-3102), evogliptin (Suganon, Evodine), gosogliptin (Saterex), and dutogliptin; (h) injectable amylin analogues such as pramlintide (Symlin); (i) glycosurics (SGLT2 inhibitors) such as canagliflozin (Invokana, Sulisent, Prominad), dapagliflozin (Forxiga, Farxiga, Edistride), empagliflozin (Jardiance, Sciampa-M), ertugliflozin (Steglatro), ipragliflozin (Suglat), luseogliflozin (Lusefi), remogliflozin etabonate (pro-drug of remogliflozin), sergliflozin etabonate (GW869682X), sotagliflozin (Zynquista), and tofogliflozin (CSG452).

In some embodiments, a compound of Formulas I, Ia, Ib, Ic, Id, and Ie can be used to treat osteoarthritis in combination with any of the following methods: (d) injections of a Wnt signaling pathway inhibitor (e.g. lorecivivint); (a) Nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g. Hyalgan, Synvisc); (e) narcotics, like codeine; (f) in combination with braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) in combination with a chronic pain class.

In some embodiments, a compound of Formulas I, Ia, Ib, Ic, Id, and Ie can be used to treat Alzheimer's disease in combination with aducanumab (Aduhelm™); acetylcholinesterase inhibitors, e.g., tacrine, rivastigmine (Exelon®), galantamine (Razadyne® and GalantaMind™), and donepezil (Aricept®); and memantine (Axura®, Ebixa®, Namenda®).

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release, or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants, and the like, for prolonged and/or timed, pulsed administration at a predetermined rate.

The compounds can be administered either alone or in combination with a conventional pharmaceutical carrier, excipient, or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol, or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the unit dosage of compounds of Formulas I, Ia, Ib, Ic, Id, and Ie is about 0.25 mg/Kg to about 50 mg/Kg in humans.

In some embodiments, the unit dosage of compounds of Formula Formulas I, Ia, Ib, Ic, Id, and Ie is about 0.25 mg/Kg to about 20 mg/Kg in humans.

In some embodiments, the unit dosage of compounds of Formula Formulas I, Ia, Ib, Ic, Id, and Ie is about 0.50 mg/Kg to about 19 mg/Kg in humans.

In some embodiments, the unit dosage of compounds of Formula Formulas I, Ia, Ib, Ic, Id, and Ie is about 0.75 mg/Kg to about 18 mg/Kg in humans.

In some embodiments, the unit dosage of compounds of Formula Formulas I, Ia, Ib, Ic, Id, and Ie is about 1.0 mg/Kg to about 17 mg/Kg in humans.

In some embodiments, the unit dosage of compounds of Formula Formulas I, Ia, Ib, Ic, Id, and Ie is about 1.25 mg/Kg to about 16 mg/Kg in humans.

In some embodiments, the unit dosage of compounds of Formula Formulas I, Ia, Ib, Ic, Id, and Ie is about 1.50 mg/Kg to about 15 mg/Kg in humans.

In some embodiments, the unit dosage of compounds of Formula Formulas I, Ia, Ib, Ic, Id, and Ie is about 1.75 mg/Kg to about 14 mg/Kg in humans.

In some embodiments, the unit dosage of compounds of Formula Formulas I, Ia, Ib, Ic, Id, and Ie is about 2.0 mg/Kg to about 13 mg/Kg in humans.

In some embodiments, the unit dosage of compounds of Formula Formulas I, Ia, Ib, Ic, Id, and Ie is about 3.0 mg/Kg to about 12 mg/Kg in humans.

In some embodiments, the unit dosage of compounds of Formula Formulas I, Ia, Ib, Ic, Id, and Ie is about 4.0 mg/Kg to about 11 mg/Kg in humans.

In some embodiments, the unit dosage of compounds of Formula Formulas I, Ia, Ib, Ic, Id, and Ie is about 5.0 mg/Kg to about 10 mg/Kg in humans.

In some embodiments, the compositions are provided in unit dosage forms suitable for single administration.

In some embodiments, the compositions are provided in unit dosage forms suitable for twice a day administration.

In some embodiments, the compositions are provided in unit dosage forms suitable for three times a day administration.

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable and could be higher if the composition is a solid or suspension, which could be subsequently diluted to the above percentages.

In some embodiments, the composition comprises about 0.1-10% of the active agent in solution.

In some embodiments, the composition comprises about 0.1-5% of the active agent in solution.

In some embodiments, the composition comprises about 0.1-4% of the active agent in solution.

In some embodiments, the composition comprises about 0.15-3% of the active agent in solution.

In some embodiments, the composition comprises about 0.2-2% of the active agent in solution.

In some embodiments, the compositions are provided in dosage forms suitable for continuous dosage by intravenous infusion over a period of about 1-96 hours.

In some embodiments, the compositions are provided in dosage forms suitable for continuous dosage by intravenous infusion over a period of about 1-72 hours.

In some embodiments, the compositions are provided in dosage forms suitable for continuous dosage by intravenous infusion over a period of about 1-48 hours.

In some embodiments, the compositions are provided in dosage forms suitable for continuous dosage by intravenous infusion over a period of about 1-24 hours.

In some embodiments, the compositions are provided in dosage forms suitable for continuous dosage by intravenous infusion over a period of about 1-12 hours.

In some embodiments, the compositions are provided in dosage forms suitable for continuous dosage by intravenous infusion over a period of about 1-6 hours.

In some embodiments, these compositions can be administered by intravenous infusion to humans at doses of about 5 mg/m² to about 300 mg/m².

In some embodiments, these compositions can be administered by intravenous infusion to humans at doses of about 5 mg/m² to about 200 mg/m².

In some embodiments, these compositions can be administered by intravenous infusion to humans at doses of about 5 mg/m² to about 100 mg/m².

In some embodiments, these compositions can be administered by intravenous infusion to humans at doses of about 10 mg/m² to about 50 mg/m².

In some embodiments, these compositions can be administered by intravenous infusion to humans at doses of about 50 mg/m² to about 200 mg/m².

In some embodiments, these compositions can be administered by intravenous infusion to humans at doses of about 75 mg/m² to about 175 mg/m².

In some embodiments, these compositions can be administered by intravenous infusion to humans at doses of about 100 mg/m² to about 150 mg/m².

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formulas I, Ia, Ib, Ic, Id, and Ie disclosed herein intended for respiratory delivery (either systemic or local) can be administered as aqueous formulations, as non-aqueous solutions, or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formulas I, Ia, Ib, Ic, Id, and Ie disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the disclosure can be incorporated into a gel formulation (e.g., U.S. Pat. Nos. 4,474,752 and 6,911,211).

In some embodiments, compounds of Formulas I, Ia, Ib, Ic, Id, and Ie disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid compound of Formulas I, Ia, Ib, Ic, Id, and Ie can be delivered from the reservoir of an external or internal implanted pumping system.

Formulations of the disclosure also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formulas I, Ia, Ib, Ic, Id, and Ie are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration of the drug (either as a solution, colloid, suspension or a complex) can be prepared by mixing a compound provided herein with a suitable non-irritating excipient that is solid at ordinary temperatures but liquid at the rectal temperature and will therefore melt or erode/dissolve in the rectum and release the compound. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter, is first melted.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of glioblastoma, ovarian, breast, pancreatic cancers, acute lymphoblastic leukemia, acute megakaryoblastic leukemia, chronic myeloid leukemia, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, Autism, Dementia, Epilepsy, Huntington's Disease, and Multiple Sclerosis.

### Methods of Treatment

The compounds and compositions provided herein can be used as inhibitors of DYRK1A, and thus can be used to treat a variety of disorders and diseases in which over expression of DYRK1A is implicated, such as cancer and neurological conditions/disorders/diseases. Non-limiting examples of diseases which can be treated with the compounds and compositions provided herein include a variety of cancers, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, tauopathies (e.g., Pick's disease, progressive supranuclear palsy, corticobasal degeneration, argyrophilic grain disease, globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

The gene encoding DYRK1A is located on chromosome 21, within the Down syndrome critical region (DSCR), the triploidy of which is responsible for most Down syndrome-associated deficiencies (FEBS Journal (2011), 278, 246-256). There is considerable genetical and pharmacological evidence showing that the mere 1.5-fold overexpression of DYRK1A is responsible for most cognitive deficits observed in Down syndrome patients (Pharmacology & Therapeutics (2019), 194, 199-221 and Brain Science (2018), 8(10), 187). Genetical normalization of DYRK1A levels or pharmacological inhibition of its catalytic activity restores cognitive functions. The development of pharmacological inhibitors of DYRK1A is a major avenue for the treatment of cognitive deficits associated with Down syndrome.

DYRK1A and DYRK1B are utilized during human cytomegalovirus (HCMV) placental replication. Inhibition of DYRKs prevent replication of various viruses, including hepatitis C virus (HCV), human cytomegalovirus (HCMV), human immunodeficiency virus type 1 (HIV-1), and herpes simplex virus 1 (HSV-1) (Journal of Virology (2020), 94(6) and PLoS ONE (2015), 10, e0144229).

There is a growing body of evidence showing that DYRK1A/1B inhibitors induce the proliferation of insulin-producing pancreatic β-cells, making DYRK1A/1B kinases attractive therapeutic targets for β-cell regeneration for both type 1 and type 2 diabetes mellitus and gestational diabetes (Nature Communications (2015), 6(8372); Diabetes (2016), 65(6), 1660-1671; JCI Insight (2020), 5(1), e132594; Science Translational Medicine (2020), 12(530); International Journal of Molecular Sciences (2021), 22(16), 9083; and Journal of Medicinal Chemistry (2021), 64(6), 2901-2922). Other forms of diabetes that may be treated with DYRK inhibitors are maturity onset diabetes of the young (MODY, monogenic diabetes), cases of diabetes that are caused by the body's tissue receptors not responding to insulin, double diabetes (when a type 1 diabetic becomes insulin resistant), diabetes associated with excessive secretion of insulin-antagonistic hormones, malnutrition-related diabetes mellitus (ICD-10 code E12), and diabetes caused by any genetic mutations (autosomal or mitochondrial) that leads to defects in beta cell function.

There is abundant literature linking DYRK1A with solid cancers and leukemias (Pharmacology & Therapeutics (2015), 151, 87-98; Cancers (2020), 12(8), 2106; and Cellular and Molecular Life Sciences (2021), 78, 603-619). The most prominent examples are pancreatic cancer (Gut (2019), 68(8), 1465-1476 and Gene (2020), 758, 144960), brain tumors, glioblastoma (Journal of Clinical Investigation (2013), 123(6), 2475-2487), acute megakaryoblastic leukemia (AMKL) (Journal of Clinical Investigation (2012), 122(3), 948-962), and acute lymphoblastic leukemia (ALL) (Journal of Clinical Investigation (2021), 131(1), e135937). Other cancers linked to DYRK1A are ovarian (Frontiers in Oncology (2021), 11, 637193), head and neck squamous cell carcinoma (Scientific Reports (2016), 6, 36132), hepatocellular carcinoma (Cell Death & Disease (2021), 12, 125), DYRK1A regulates DNA damage response (Scientific Reports (2019), 9, 6014 and Scientific Reports (2019), 9, 6539). In some situations, DYRK1A appears to function as a tumor-suppressor protein (Molecular & Cellular Oncology (2015), 2(1), e970048 and Nature (2016), 529, 172-177).

Other cancers can also be treated with the compounds and compositions described herein.

More particularly, cancers that may be treated by the compounds, compositions and methods described herein include, but are not limited to, the following:

1) Breast cancers, including, for example ER⁺ breast cancer, ER⁻ breast cancer, her2⁻ breast cancer, her2⁺ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; chemoresistant breast cancers (TNBC), and miscellaneous malignant neoplasms. Further examples of breast cancers can include luminal A, luminal B, basal A, basal B, and triple negative breast cancer, which is estrogen receptor negative (ER⁻), progesterone receptor negative, and her2 negative (her2⁻). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; chemoresistant small cell lung cancer (SCLC), and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; colon cancers with APC gene mutations; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, oligodendrocytoma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial cancers (e.g., carcinoma, endometrioid adenocarcinoma, serous carcinoma, clear cell carcinoma, mucinous carcinomas, mixed or undifferentiated carcinoma (including mixed Mullerian tumor), endometrial stromal sarcoma, squamous cell carcinoma of the endometrium, urothelial carcinoma, endometrial cancer with CTNNB1 mutations); cancers of the cervix, e.g., cervical carcinoma, and pre tumor cervical dysplasia; cancers of the ovaries, e.g., BRCA-mutant ovarian cancer, surface epithelial-stromal tumors (epithelial ovarian cancer (Type 1 (endometroid, mucinous, clear cell, low grade serous) or Type 2 (poorly differentiated, carcinosarcoma, and high grade serous))), ovarian carcinoma, including serous cystadenocarcinoma, mucinous cystadenocarcinoma, endometrioid tumors, small cell ovarian cancer (small cell ovarian cancer of hypercalcemic type, small cell ovarian cancer of pulmonary type) unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma, primary fallopian tube cancer; Primary peritoneal cancer (also known as serous surface papillary carcinoma, primary peritoneal carcinoma, extra-ovarian serous carcinoma, primary serous papillary carcinoma, and psammomacarcinoma).

10) Hematologic cancers, including, for example, cancers of the blood, e.g., acute myeloid leukemia, chronic myeloid leukemia, myelodysplastic syndromes (refractory cytopenia with unilineage dysplasia (refractory anemia, refractory neutropenia, and refractory thrombocytopenia), refractory anemia with ring sideroblasts, refractory cytopenia with multilineage dysplasia, refractory anemias with excess blasts I and II, refractory cytopenia of childhood), and myeloproliferative neoplasms, acute lymphoblastic leukemia, chronic lymphocytic leukemia, myeloproliferative diseases, multiple myeloma, myelodysplastic syndrome, myelodysplastic-myeloproliferative diseases, Hodgkin's lymphoma, non-Hodgkin's lymphoma (malignant lymphoma) and Waldenström's macroglobulinemia.

11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.

12) Adrenal gland cancers, including, for example, neuroblastoma.

13) Soft-tissue sarcomas (STS) such as fibrosarcoma, malignant fibrous histiocytoma, dermatofibrosarcoma, liposarcoma, rhabdomyosarcoma, leiomyosarcoma, hemangiosarcoma, Kaposi's sarcoma, lymphangiosarcoma, synovial sarcoma, malignant peripheral nerve sheath tumors (also called neurofibrosarcomas, malignant schwannomas, and neurogenic sarcomas), neurofibrosarcoma, extraskeletal chondrosarcoma, extraskeletal osteosarcoma, extraskeletal myxoid chondrosarcoma, extraskeletal mesenchymal, embryonal, alveolar soft part sarcoma, and infantile hemangio-pericytoma.

More particularly, tumors of the central nervous system that may be treated by the compounds, compositions and methods described herein include:

1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.

2) Oligodendroglial tumors, e.g., oligodendroglioma and anaplastic oligodendroglioma.

3) Oligoastrocytic tumors, e.g., oligoastrocytoma and anaplastic oligoastrocytoma.

4) Ependymal tumors, e.g., subependymoma, myxopapillary ependymoma, ependymoma, (cellular, papillary, clear cell, tanycytic), and anaplastic (malignant) ependymoma.

5) Choroid plexus tumors, e.g., choroid plexus papilloma, atypical choroid plexus papilloma, and choroid plexus carcinoma.

6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.

7) Pineal tumors, e.g., pineocytoma, pineoblastoma, papillary tumors of the pineal region, and pineal parenchymal tumor of intermediate differentiation.

8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.

9) Neuroblastic tumors, e.g., olfactory (esthesioneuroblastoma), olfactory neuroepithelioma, and neuroblastomas of the adrenal gland and sympathetic nervous system.

10) Glial tumors, e.g., astroblastoma, chordoid glioma of the third ventricle, and angiocentric glioma.

11) Tumors of cranial and paraspinal nerves, e.g., schwannoma, neurofibroma Perineurioma, and malignant peripheral nerve sheath tumor.

12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.

13) Tumors of the hematopoietic system, e.g., malignant Lymphomas, plasmocytoma, and granulocytic sarcoma.

14) Germ cell tumors, e.g., germinoma, embryonal carcinoma, yolk sac tumor, choriocarcinoma, teratoma, and mixed germ cell tumors.

15) Tumors of the sellar region, e.g., craniopharyngioma, granular cell tumor, pituicytoma, and spindle cell oncocytoma of the adenohypophysis.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

There is mounting evidence for a role of DYRK1A in the onset of Alzheimer's Disease (Future Medicinal Chemistry (2016), 8(6), 681-696 and European Journal of Medicinal Chemistry (2018), 158, 559-592). DYRK1A phosphorylates key substrates involved in Alzheimer's Disease and dementia: Tau, septin 4, amyloid precursor protein (APP), presenilin 1, neprilysin, Munc18-1, α-synuclein, RCAN1, and β-tubulin. By modulating alternative splicing of Tau exon 10, DYRK1A favors the production of the 3R-Tau splice isoform (characteristic for DS/AD/tauopathy) over the 4R-Tau isoform (Journal of Biological Chemistry (2015), 290, 15219-15237).

Genome-wide association studies (GWAS) have revealed that DYRK1A is a risk factor for Parkinson's Disease (The Lancet Neurology (2019), 18(12), 1091-1102). DYRK1A phosphorylates key factors for Parkinson's Disease such as parkin, septin 4, and α-synuclein. Upregulation of micro-RNAs specific for Parkinson's Disease targets DYRK1A expression. There is further evidence that DYRK1A expression is increased in Parkinson's Disease and in Pick's disease (Neurobiology of Disease (2005), 20(2), 392-400).

The compounds and compositions provided herein can be used as inhibitors and/or modulators of the enzyme DYRK1A, and thus can be used to treat a variety of disorders and diseases associated with tau protein, including, but not limited to, Alzheimer's disease, amyotrophic lateral sclerosis (ALS), down syndrome, frontotemporal dementia (FTD) including FTD with Parkinsonism-17 (FTDP-17), behavioural variant frontotemporal dementia (bvFTD), FTD in patients with motor neuron disease (MND) (typically amyotrophic lateral sclerosis, also called FTD-ALS), corticobasal degeneration (CBD) (also called corticobasal ganglionic degeneration), progressive supranuclear palsy, primary progressive aphasia (PPA), globular glial tauopathy (GGT), myotonic dystrophy type 1 (DM1) (also called Steinert disease), myotonic dystrophy type 2 (DM2) (also called proximal myotonic myopathy), Guam complex, argyrophilic grain disease, dementia pugilistica, post-encephalitic parkinsonism, Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

Non-limiting examples of neurological disorders (e.g., neurological conditions and neurological diseases) which can be treated with the compounds and compositions provided herein include Alzheimer's disease, aphasia, apraxia, arachnoiditis, ataxia telangiectasia, attention deficit hyperactivity disorder, auditory processing disorder, autism, alcoholism, Bell's palsy, bipolar disorder, brachial plexus injury, Canavan disease, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, cerebral vasculitis, cervical spinal stenosis, Charcot-Marie-Tooth disease, Chiari malformation, chronic fatigue syndrome, chronic inflammatory demyelinating polyneuropathy (CIDP), chronic pain, Coffin-Lowry syndrome, complex regional pain syndrome, compression neuropathy, congenital facial diplegia, corticobasal degeneration, cranial arteritis, craniosynostosis, Creutzfeldt-Jakob disease, cumulative trauma disorder, Cushing's syndrome, cytomegalic inclusion body disease (CIBD), Dandy-Walker syndrome, Dawson disease, De Morsier's syndrome, Dejerine-Klumpke palsy, Dejerine-Sottas disease, delayed sleep phase syndrome, dementia, dermatomyositis, developmental dyspraxia, diabetic neuropathy, diffuse sclerosis, Dravet syndrome, dysautonomia, dyscalculia, dysgraphia, dyslexia, dystonia, empty sella syndrome, encephalitis, encephalocele, encephalotrigeminal angiomatosis, encopresis, epilepsy, Erb's palsy, erythromelalgia, essential tremor, Fabry's disease, Fahr's syndrome, familial spastic paralysis, febrile seizure, Fisher syndrome, Friedreich's ataxia, fibromyalgia, Foville's syndrome, Gaucher's disease, Gerstmann's syndrome, giant cell arteritis, giant cell inclusion disease, globoid cell leukodystrophy, gray matter heterotopia, Guillain-Barré syndrome, HTLV-1 associated myelopathy, Hallervorden-Spatz disease, hemifacial spasm, hereditary spastic paraplegia, heredopathia atactica polyneuritiformis, herpes zoster oticus, herpes zoster, Hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile Refsum disease, infantile spasms, inflammatory myopathy, intracranial cyst, intracranial hypertension, Joubert syndrome, Karak syndrome, Kearns-Sayre syndrome, Kennedy disease, Kinsbourne syndrome, Klippel Feil syndrome, Krabbe disease, Kugelberg-Welander disease, kuru, Lafora disease, Lambert-Eaton myasthenic syndrome, Landau-Kleffner syndrome, lateral medullary (Wallenberg) syndrome, Leigh's disease, Lennox-Gastaut syndrome, Lesch-Nyhan syndrome, leukodystrophy, Lewy body dementia, lissencephaly, locked-in syndrome, Lou Gehrig's disease, lumbar disc disease, lumbar spinal stenosis, Lyme disease, Machado-Joseph disease (Spinocerebellar ataxia type 3), macrencephaly, macropsia, megalencephaly, Melkersson-Rosenthal syndrome, Meniere's disease, meningitis, Menkes disease, metachromatic leukodystrophy, microcephaly, micropsia, Miller Fisher syndrome, misophonia, mitochondrial myopathy, Mobius syndrome, monomelic amyotrophy, motor neuron disease, motor skills disorder, Moyamoya disease, mucopolysaccharidoses, multi-infarct dementia, multifocal motor neuropathy, multiple sclerosis, multiple system atrophy, muscular dystrophy, myalgic encephalomyelitis, myasthenia gravis, myelinoclastic diffuse sclerosis, myoclonic Encephalopathy of infants, myoclonus, myopathy, myotubular myopathy, myotonia congenital, narcolepsy, neurofibromatosis, neuroleptic malignant syndrome, lupus erythematosus, neuromyotonia, neuronal ceroid lipofuscinosis, Niemann-Pick disease, O'Sullivan-McLeod syndrome, occipital Neuralgia, occult Spinal Dysraphism Sequence, Ohtahara syndrome, olivopontocerebellar atrophy, opsoclonus myoclonus syndrome, optic neuritis, orthostatic hypotension, palinopsia, paresthesia, Parkinson's disease, paramyotonia Congenita, paraneoplastic diseases, paroxysmal attacks, Parry-Romberg syndrome, Pelizaeus-Merzbacher disease, periodic paralyses, peripheral neuropathy, photic sneeze reflex, phytanic acid storage disease, Pick's disease, polymicrogyria (PMG), polymyositis, porencephaly, post-polio syndrome, postherpetic neuralgia (PHN), postural hypotension, Prader-Willi syndrome, primary lateral sclerosis, prion diseases, progressive hemifacial atrophy, progressive multifocal leukoencephalopathy, progressive supranuclear palsy, pseudotumor cerebri, Ramsay Hunt syndrome type I, Ramsay Hunt syndrome type II, Ramsay Hunt syndrome type III, Rasmussen's encephalitis, reflex neurovascular dystrophy, Refsum disease, restless legs syndrome, retrovirus-associated myelopathy, Rett syndrome, Reye's syndrome, rhythmic movement disorder, Romberg syndrome, Saint Vitus dance, Sandhoff disease, schizophrenia, Schilder's disease, schizencephaly, sensory integration dysfunction, septo-optic dysplasia, Shy-Drager syndrome, Sjögren's syndrome, snatiation, Sotos syndrome, spasticity, spina bifida, spinal cord tumors, spinal muscular atrophy, spinocerebellar ataxia, Steele-Richardson-Olszewski syndrome, Stiff-person syndrome, stroke, Sturge-Weber syndrome, subacute sclerosing panencephalitis, subcortical arteriosclerotic encephalopathy, superficial siderosis, Sydenham's chorea, syncope, synesthesia, syringomyelia, tarsal tunnel syndrome, tardive dyskinesia, tardive dysphrenia, Tarlov cyst, Tay-Sachs disease, temporal arteritis, tetanus, tethered spinal cord syndrome, Thomsen disease, thoracic outlet syndrome, tic douloureux, Todd's paralysis, Tourette syndrome, toxic encephalopathy, transient ischemic attack, transmissible spongiform encephalopathies, transverse myelitis, tremor, trigeminal neuralgia, tropical spastic paraparesis, trypanosomiasis, tuberous sclerosis, ubisiosis, Von Hippel-Lindau disease (VHL), Viliuisk Encephalomyelitis (VE), Wallenberg's syndrome, Werdnig, Hoffman disease, west syndrome, Williams syndrome, Wilson's disease, and Zellweger syndrome.

The compounds and compositions may also be useful in the inhibition of the development of invasive cancer, tumor angiogenesis and metastasis.

In some embodiments, the pharmaceutical composition comprises a therapeutically effective amount of a compound of Formulas I, Ia, Ib, Ic, Id, and Ie, or a pharmaceutically acceptable salt thereof, and a pharmaceutically acceptable excipient.

In some embodiments, the disorder or disease is cancer.

In some embodiments, the disorder or disease is metastatic melanoma.

In some embodiments, the disorder or disease is tendon regeneration.

In some embodiments, the disorder or disease is diabetes.

In some embodiments, the disorder or disease is degenerative disc disease.

In some embodiments, the disorder or disease is osteoarthritis.

In some embodiments, the disorder or disease is a viral infection.

In some embodiments, the disorder or disease is a neurological disorder.

In some embodiments, the disorder or disease is Alzheimer's disease.

In some embodiments, the disorder or disease is osteoarthritis.

In some embodiments, the patient is a human.

In some embodiments, the cancer is chosen from: hepatocellular carcinoma, colon cancer, breast cancer, pancreatic cancer, chronic myeloid leukemia (CML), chronic myelomonocytic leukemia, chronic lymphocytic leukemia (CLL), acute myeloid leukemia, acute lymphocytic leukemia, Hodgkin lymphoma, lymphoma, sarcoma, and ovarian cancer.

In some embodiments, the cancer is chosen from: lung cancer—non-small cell, lung cancer—small cell, multiple myeloma, nasopharyngeal cancer, neuroblastoma, osteosarcoma, penile cancer, pituitary tumors, prostate cancer, retinoblastoma, synovial sarcoma, rhabdomyosarcoma, salivary gland cancer, skin cancer—basal and squamous cell, skin cancer—melanoma, small intestine cancer, stomach (gastric) cancers, testicular cancer, thymus cancer, thyroid cancer, uterine sarcoma, vaginal cancer, vulvar cancer, laryngeal or hypopharyngeal cancer, kidney cancer, Kaposi sarcoma, gestational trophoblastic disease, gastrointestinal stromal tumor, gastrointestinal carcinoid tumor, gallbladder cancer, eye cancer (melanoma and lymphoma), Ewing tumor, esophagus cancer, endometrial cancer, colorectal cancer, cervical cancer, brain or spinal cord tumor, bone metastasis, bone cancer, bladder cancer, bile duct cancer, anal cancer and adrenal cortical cancer.

In some embodiments, the cancer is hepatocellular carcinoma; in some embodiments, the cancer is colon cancer; in some embodiments, the cancer is colorectal cancer; in some embodiments, the cancer is breast cancer; in some embodiments, the cancer is pancreatic cancer; in some embodiments, the cancer is chronic myeloid leukemia (CML); in some embodiments, the cancer is chronic myelomonocytic leukemia; in some embodiments, the cancer is chronic lymphocytic leukemia (CLL); in some embodiments, the cancer is acute myeloid leukemia; in some embodiments, the cancer is acute lymphocytic leukemia; in some embodiments, the cancer is Hodgkin lymphoma; in some embodiments, the cancer is lymphoma; in some embodiments, the cancer is sarcoma; in some embodiments, the cancer is ovarian cancer; in some embodiments, the cancer is lung cancer—non-small cell; in some embodiments, the cancer is lung cancer—small cell; in some embodiments, the cancer is multiple myeloma; in some embodiments, the cancer is nasopharyngeal cancer; in some embodiments, the cancer is neuroblastoma; in some embodiments, the cancer is osteosarcoma; in some embodiments, the cancer is penile cancer; in some embodiments, the cancer is pituitary tumors; in some embodiments, the cancer is prostate cancer; in some embodiments, the cancer is retinoblastoma; in some embodiments, the cancer is rhabdomyosarcoma; in some embodiments, the cancer is salivary gland cancer; in some embodiments, the cancer is skin cancer—basal and squamous cell; in some embodiments, the cancer is skin cancer—melanoma; in some embodiments, the cancer is small intestine cancer; in some embodiments, the cancer is stomach (gastric) cancers; in some embodiments, the cancer is testicular cancer; in some embodiments, the cancer is thymus cancer; in some embodiments, the cancer is thyroid cancer; in some embodiments, the cancer is uterine sarcoma; in some embodiments, the cancer is vaginal cancer; in some embodiments, the cancer is vulvar cancer; in some embodiments, the cancer is Wilms tumor; in some embodiments, the cancer is laryngeal or hypopharyngeal cancer; in some embodiments, the cancer is kidney cancer; in some embodiments, the cancer is Kaposi sarcoma; in some embodiments, the cancer is gestational trophoblastic disease; in some embodiments, the cancer is gastrointestinal stromal tumor; in some embodiments, the cancer is gastrointestinal carcinoid tumor; in some embodiments, the cancer is gallbladder cancer; in some embodiments, the cancer is eye cancer (melanoma and lymphoma); in some embodiments, the cancer is Ewing tumor; in some embodiments, the cancer is esophagus cancer; in some embodiments, the cancer is endometrial cancer; in some embodiments, the cancer is colorectal cancer; in some embodiments, the cancer is cervical cancer; in some embodiments, the cancer is brain or spinal cord tumor; in some embodiments, the cancer is bone metastasis; in some embodiments, the cancer is bone cancer; in some embodiments, the cancer is bladder cancer; in some embodiments, the cancer is bile duct cancer; in some embodiments, the cancer is anal cancer; and in some embodiments, the cancer is adrenal cortical cancer.

In some embodiments, the cancer is the cancer is selected from the group consisting of: brain tumors, glioblastoma, ovarian, breast, head and neck squamous cell carcinoma, hepatocellular carcinoma, pancreatic cancer, acute lymphoblastic leukemia, acute megakaryoblastic leukemia, and chronic myeloid leukemia.

In some embodiments, the disorder or disease is a neurological condition, disorder, or disease, wherein the neurological disease is selected from: Alzheimer's disease, frontotemporal dementias, Parkinson's disease, Huntington's disease, progressive supranuclear palsy, corticobasal degeneration, multiple system atrophy, amyotrophic lateral sclerosis (ALS), inclusion body myositis, autism, degenerative myopathies.

In some embodiments, the disorder or disease is selected from the group consisting of: Alzheimer's Disease, Amyotrophic Lateral Sclerosis, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, and Stroke.

In some embodiments, the disorder or disease is a neurological disorder, wherein the neurological disorder is selected from the group consisting of: Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, Pick disease, progressive supranuclear palsy, corticobasal degeneration, argyrophilic grain disease, globular glial tauopathies, primary age-related tauopathy, neurofibrillary tangle dementia, chronic traumatic encephalopathy (CTE), frontotemporal lobar degeneration with tau inclusions (FTLD-tau), and aging-related tau astrogliopathy.

In some embodiments, the disorder or disease is Alzheimer's disease.

In some embodiments, a compound of Formulas I, Ia, Ib, Ic, Id, and Ie inhibits DYRK1A.

In some embodiments, the method treats a disease or disorder mediated by kinase activity in a patient, the method comprises administering to the patient a therapeutically effective amount of a compound (or compounds) of Formulas I, Ia, Ib, Ic, Id, and Ie, or a pharmaceutically acceptable salt thereof.

In some embodiments, the disease or disorder comprises tumor growth, cell proliferation, or angiogenesis.

In some embodiments, the method inhibits the activity of a protein kinase receptor, the method comprises contacting the receptor with an effective amount of a compound (or compounds) of Formulas I, Ia, Ib, Ic, Id, and Ie, or a pharmaceutically acceptable salt thereof.

In some embodiments, the method treats a disease or disorder associated with aberrant cellular proliferation in a patient; the method comprises administering to the patient a therapeutically effective amount of a compound (or compounds) of Formulas I, Ia, Ib, Ic, Id, and Ie, or a pharmaceutically acceptable salt thereof.

In some embodiments, the method prevents or reduces abnormal cellular proliferation in a patient; the method comprises administering to the patient a therapeutically effective amount of a compound (or compounds) of Formulas I, Ia, Ib, Ic, Id, and Ie, or a pharmaceutically acceptable salt thereof.

In some embodiments, the method treats a disease or disorder associated with aberrant cellular proliferation in a patient, the method comprises administering to the patient a pharmaceutical composition comprising one or more of the compounds of claim 1 in combination with a pharmaceutically acceptable carrier and one or more other agents.

### Evaluation of Biological Activity

The biological activity of the compounds described herein can be tested using any suitable assay known to those of skill in the art. For example, the activity of a compound may be tested using one or more of the test methods outlined below.

For example, in vitro assays for DYRK1A biological activity may be used, e.g., regulation of microtubule-associated protein tau (MAPT/Tau) phosphorylation in neuronal cell lines such as the human SH-SY5Y neuroblastoma cell line. Assays for DYRK1A-regulated level of phosphorylation can include monitoring levels of basal pSer396 Tau, which can be measured, for example, by serial dilutions of a candidate inhibitor composition using a ten micromolar top concentration and detected by ELISA or Western Blotting. An exemplary assay for DYRK-1A-regulated phosphorylation uses the SH-SY5Y cells cultured in a 96 well plate format for a period of time sufficient to stabilize microtubules and Tau phosphorylation, usually at least 2 days, then treated with a ⅓ serial dilution of compounds overnight and lysed. The cell lysate is resolved by SDS PAGE, then transferred to nitrocellulose and probed with an antibody specific for pSer396 Tau. The chemiluminescence signal for HRP-linked antibodies used in western blotting is detected using a Carestream Image Station and blot densitometry for pSer396 and beta-actin are analyzed using ImageJ (NIH).

In a further example, the activity of a candidate compound can be measured by phosphoTau (Thr212) AlphaLISA by adding the lysate mentioned above onto total Tau-coated plates and detected with a specific pThr212Tau antibody. Colorimetric detection of AlphaLISA signal is performed by EnVision Multilabel Plate Reader (Perkin Elmer).

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described and claimed herein. The reader will recognize that the skilled artisan, armed with the present disclosure, and skill in the art is able to prepare and use the disclosure without exhaustive examples.

### EXAMPLES

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety)and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

(¹H) nuclear magnetic resonance spectra (NMR) were measured in the indicated solvents on a Bruker NMR spectrometer (Avance TM DRX300, 300 MHz for ¹H or Avance TM DRX500, 500 MHz for ¹H) or Varian NMR spectrometer (Mercury 400BB, 400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; ddd, doublet of doublets of doublets; d/ABq, doublet of AB quartet; dt, doublet of triplets; td, triplet of doublets; dq, doublet of quartets; m, multiplet.

The following abbreviations have the indicated meanings:

Ac₂O=acetic anhydride

BrettPhos=2-(dicyclohexylphosphino)3,6-dimethoxy-2′,4′,6′-triisopropyl-1,1′-biphenyl

BrettPhos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate

brine=saturated aqueous sodium chloride

CDCl₃=deuterated chloroform

DCE=dichloroethane

DCM=dichloromethane

DIPEA=N,N-diisopropylethylamine

DMA=dimethylacetamide

DMAP=4-dimethylaminopyridine

DME=dimethoxyethane

DMF=N,N-dimethylformamide

DMSO-d₆=deuterated dimethylsulfoxide

ESIMS=electron spray mass spectrometry

EtOAc=ethyl acetate

HATU=1-[Bis(dimethylamino)methylene]-1H-1,2,3-triazolo[4,5-b]pyridinium 3-oxid hexafluorophosphate

HCl=hydrochloric acid

HOAc=acetic acid

IPA=isopropyl alcohol

ISCO=Teledyne ISCO, Inc brand CombiFlash® Rf 200

KOAc=potassium acetate

LAH=Lithium aluminum hydride

LC/MS=Liquid chromatography-mass spectrometry

=lithium bis(trimethylsilyl)amide

MeCN=acetonitrile

MeOH=methanol

MgSO₄=magnesium sulfate

MsCl=mesyl chloride or methanesulfonyl chloride

MTBE=methyl tert-butyl ether

MW=microwave irradiation

NaBH₃CN=sodium cyanoborohydride

NaHCO₃=sodium bicarbonate

NaBH(OAc)₃=Sodium triacetoxyborohydride

NMR=nuclear magnetic resonance

ON=overnight

Pd₂(dba)₃=tris(dibenzylideneacetone)dipalladium(0)

Pd(dppf)Cl₂=1,1′-bis(diphenylphosphino)ferrocene-palladium(II)dichloride

Pd(PPh₃)₄=tetrakis(triphenylphosphine)palladium(0)

PPTS=pyridinium p-toluenesulfonate

r.t.=room temperature

SPhos Pd G3=[(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate

SPhos Pd G4=Methanesulfonato(2-dicyclohexylphosphino-3,6-dimethoxy-2′,4′,6′-tri-i-propyl-1,1′-biphenyl)(2′-methylamino-1,1′-biphenyl-2-yl)palladium(II)

TBAF=Tetra-n-butylammonium fluoride,

TEA=triethylamine

TFA=trifluoroacetic acid

THF=tetrahydrofuran

TLC=thin layer chromatography

X-PHOS=2-Dicyclohexylphosphino-2′,4′,6′-triisopropylbiphenyl

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application. Unless otherwise indicated, all variables are as defined above.

### General Procedures

Compounds of Formulas I and Ia of the present disclosure can be prepared as depicted in Scheme 1.

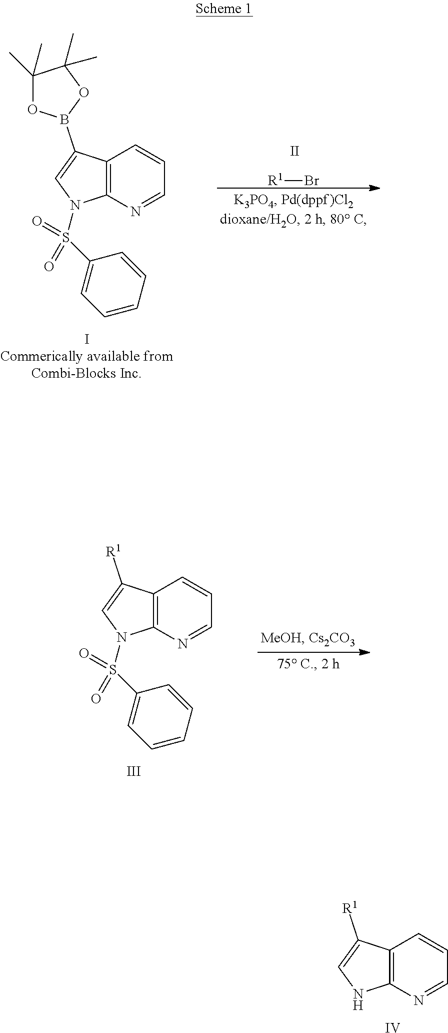

Scheme 1 describes a method for preparation of 1H-pyrrolo[2,3-b]pyridine derivatives (IV) by first reacting the boronic acid pinacol ester (I) using Suzuki coupling with a variety of bromines (II) to produce protected 1H-pyrrolo[2,3-b]pyridine III. Deprotection of the phenylsulfonyl protected N with Cs₂CO₃ produces the final R¹ substituted 1H-pyrrolo[2,3-b]pyridine IV.

In some embodiments, compounds of Formulas I, Ib, and Ic of the present disclosure can be prepared as depicted in Scheme 2.

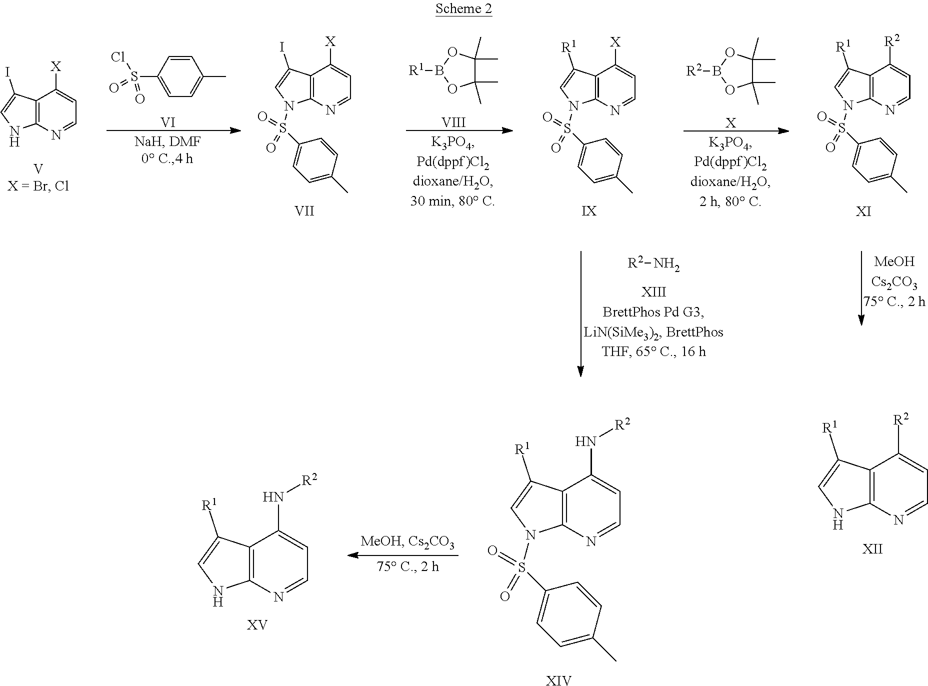

Scheme 2 describes a method for preparation of R² substituted 1H-pyrrolo[2,3-b]pyridine derivatives (XII) and (XV) by first protecting the pyrrolo N of compound V with tosyl chloride to form VII. Suzuki coupling of the iodo (VII) with various boronic acid pinacol esters (VIII) yield derivatives IX. The halide on compound IX can be reacted with various boronic acid pinacol esters (X) produces protected compounds (XI) which after deprotection yields 1H-pyrrolo[2,3-b]pyridine derivatives (XII). Alternatively, the halide on compound IX can be coupled with a variety of amines (XIII) to give derivatives (XIV) which after deprotection yields 1H-pyrrolo[2,3-b]pyridine derivatives (XV).

In other embodiments, compounds of Formulas I and Ie of the present disclosure can be prepared as depicted in Scheme 3.

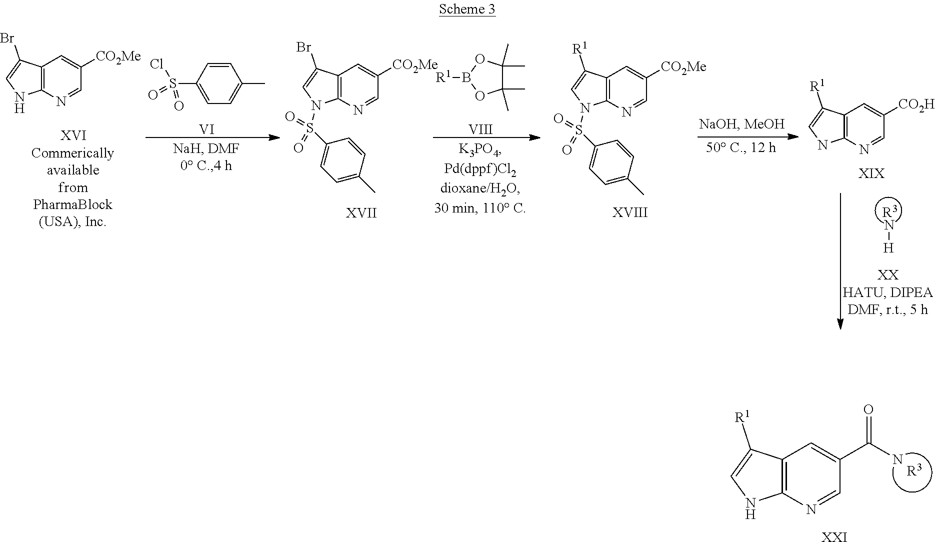

Scheme 3 describes a method for preparation of R³ substituted 1H-pyrrolo[2,3-b]pyridine derivatives (XXI) by first protecting the pyrrolo N of compound XVI with tosyl chloride to form XVII. Suzuki coupling of bromine (XVII) with various boronic acid pinacol esters (VIII) yields derivatives XVIII. The ester is hydrolyzed using NaOH which also removes the tosylate to give acid XIX. The acid is then coupled with a variety of amines to produce R³ substituted 1H-pyrrolo[2,3-b]pyridine derivatives (XXI).

In other embodiments, compounds of Formulas I and Ie of the present disclosure can be prepared as depicted in Scheme 4.

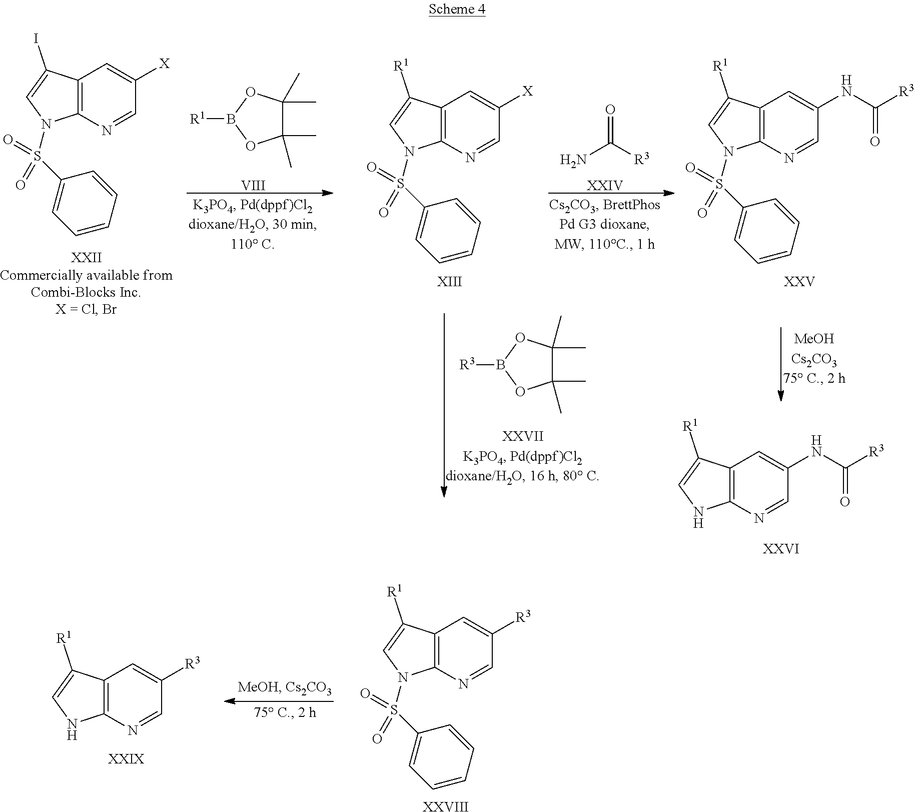

Scheme 4 describes a method for preparation of additional R³ substituted 1H-pyrrolo[2,3-b]pyridine derivatives (XXVI) and (XXIX) by first reacting the iodo compound (XXII) using Suzuki coupling with a variety of boronic acid pinacol esters (VIII) to produce protected 1H-pyrrolo[2,3-b]pyridine XIII. The halide of compound XIII can be coupled with a variety of primary amides (XXIV) to give derivative XXV which after deprotection yields R³ substituted 1H-pyrrolo[2,3-b]pyridine derivatives (XXVI). Alternatively, the halide on compound XIII can be coupled with a variety of boronic acid pinacol esters (XXVII) to give derivatives (XXVIII) which after deprotection yields R³ substituted 1H-pyrrolo[2,3-b]pyridine derivatives (XXIX).

In other embodiments, compounds of Formulas I, Ib, and Id of the present disclosure can be prepared as depicted in Scheme 5.

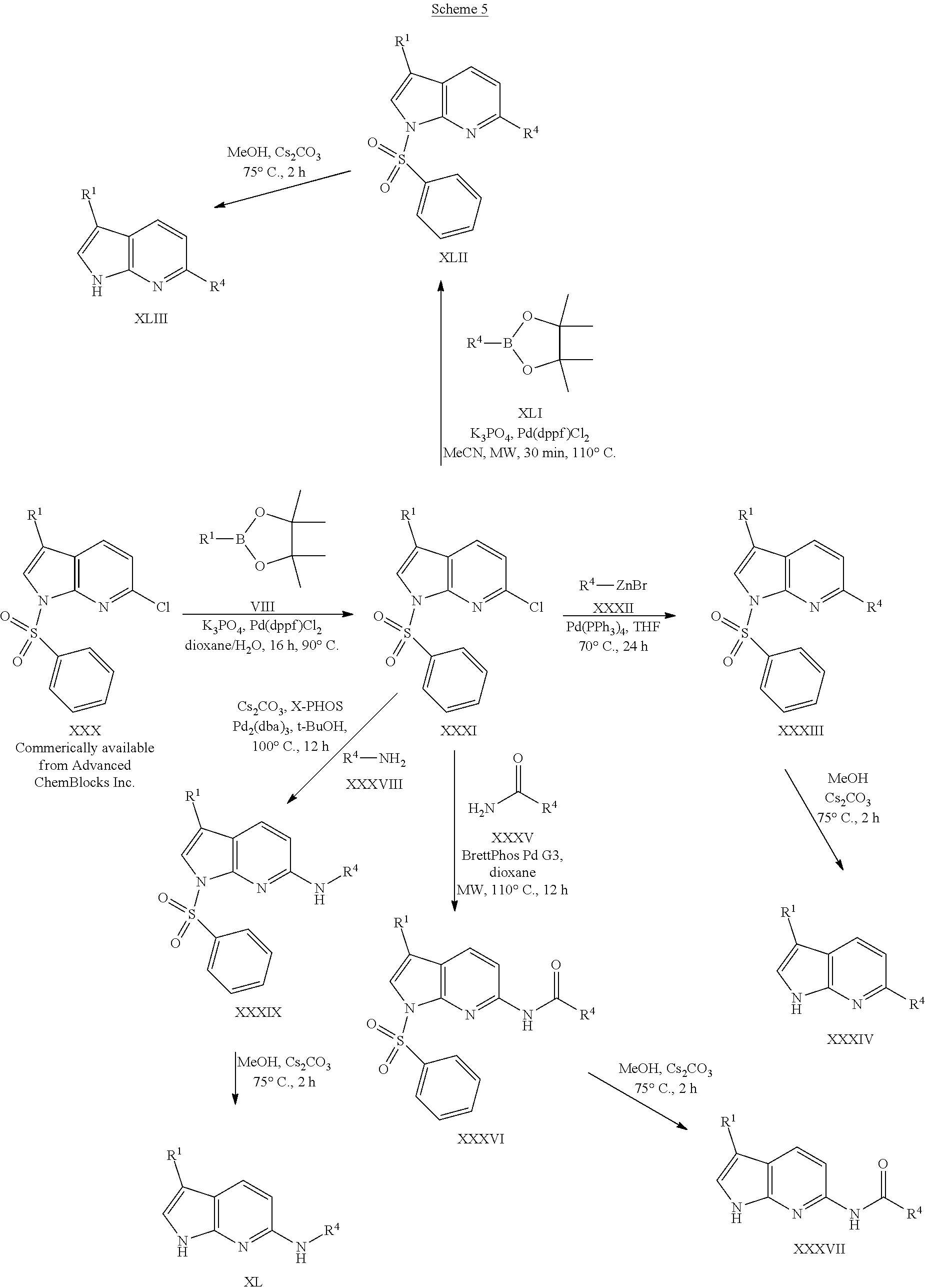

Scheme 5 describes a method for preparation of additional R⁴ substituted 1H-pyrrolo[2,3-b]pyridine derivatives (XXXIV), (XXXVII), (XL), and (XLIII) by first reacting the bromine compound (XXX) using Suzuki coupling with a variety of boronic acid pinacol esters (VIII) to produce protected 1H-pyrrolo[2,3-b]pyridine XXXI. Negishi coupling of the chloride with a variety of organozinc compounds (XXXII) produce derivative XXV which after deprotection yields R⁴ substituted 1H-pyrrolo[2,3-b]pyridine derivatives (XXXIV). Alternatively, the halide on compound XXXI can be coupled with a variety of primary amides (XXXV) to give derivative XXXVI which after deprotection yields the R⁴ substituted 1H-pyrrolo[2,3-b]pyridine derivatives (XXXVII). A third route couples the halide (XXXI) with a variety of amines to produce derivative (XXXIX) which after deprotection yields the R⁴ substituted 1H-pyrrolo[2,3-b]pyridine derivatives (XL). A fourth route couples the halide (XXXI) with a variety of various boronic acid pinacol esters (XLI) to produce derivative (XLII) which after deprotection gives the R⁴ substituted 1H-pyrrolo[2,3-b]pyridine derivatives (XLIII).

### Illustrative Compound Examples

Preparation of intermediate 6-(difluoromethoxy)-3-iodo-1H-pyrrolo[2,3-b]pyridine (XLVI) is depicted below in Scheme 6.

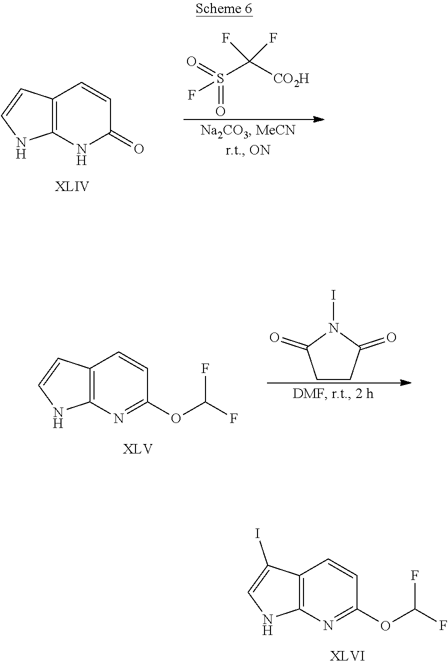 [SMILES: O=c1ccc2cc[nH]c2[nH]1 ; O=C(O)C(F)(F)S(=O)(=O)F ; FC(F)Oc1ccc2cc[nH]c2n1 ; O=C1CCC(=O)N1I ; FC(F)Oc1ccc2c(I)c[nH]c2n1]

### Step 1

To a stirred suspension of 1,7-dihydropyrrolo[2,3-b]pyridin-6-one (XVIV) (commercially available from AstaTech Inc.) (0.5 g, 3.73 mmol) and 2,2-difluoro-2-(fluorosulfonyl)acetic acid (commercially available from AK Scientific, Inc.) (0.46 mL, 4.5 mmol) in MeCN (10 mL) was added under N₂, Na₂CO₃ (1.01 g, 9.32 mmol) (exothermic!). The mixture was stirred at room temperature overnight. The reaction mixture was added to a saturated aqueous NaHCO₃ solution and extracted with CHCl₃. The organic layer was separated, washed with water and brine. The crude product was purified by ISCO (0→40% EtOAc/hexanes) to obtain 6-(difluoromethoxy)-1H-pyrrolo[2,3-b]pyridine (XLV) (442 mg, 2.400 mmol, 64.4% yield) as a white solid. ESIMS found for C₈H₆F₂N₂O m/z 185.0 (M+H).

### Step 2

To a stirred solution of 6-(difluoromethoxy)-1H-pyrrolo[2,3-b]pyridine (XLV) (0.4 g, 2.17 mmol) in DMF (6 mL) was portion-wise added N-iodosuccinimide (0.49 g, 2.18 mmol) at 0° C. The reaction mixture was stirred at room temperature for 2 h. The reaction mixture was poured into water (75 mL), stirred for 1 h and the solids were collected by filtration, washed with water, and air-dried under vacuo overnight to obtain 6-(difluoromethoxy)-3-iodo-1H-pyrrolo[2,3-b]pyridine (XLVI) (631 mg, 2.035 mmol, 93.7% yield) as a beige solid which is used for next step without purification. ESIMS found for C₈H₅F₂IN₂O m/z 310.9 (M+H).

Preparation of intermediate 6-ethoxy-3-iodo-1H-pyrrolo[2,3-b]pyridine (XLVIII) is depicted below in Scheme 7.

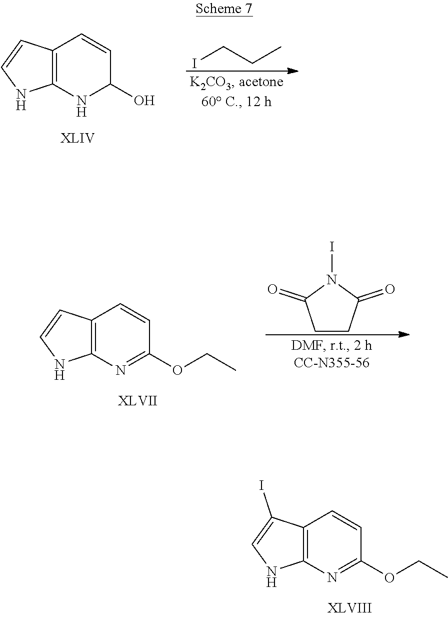

### Step 1

A mixture of 1H-pyrrolo[2,3-b]pyridin-6-ol (XLIV) (commercially available from Advanced ChemBlocks Inc.) (2.03 g, 15.13 mmol), K₂CO₃ (6.3 g, 45.58 mmol) and iodoethane (1.3 mL, 16.17 mmol) in acetone (30 mL) was refluxed at 60° C. for 12 h. The reaction mixture was cooled, filtered through celite®, and washed with EtOAc. The filtrate was concentrated, and the residue was purified by column chromatography (0→5% MeOH/CHCl₃) to obtain 6-ethoxy-1H-pyrrolo[2,3-b]pyridine (XLVII) (1.44 g, 8.879 mmol, 58.7% yield) as a brown solid. ESIMS found for C₉H₁₀N₂O m/z 163.1 (M+H).

### Step 2

To a stirred suspension of 6-ethoxy-1H-pyrrolo[2,3-b]pyridine (XLVII) (1.44 g, 8.88 mmol) in DMF (20 mL) was added portion wise 1-iodopyrrolidine-2,5-dione (2 g, 8.89 mmol) and the mixture was stirred at room temperature for 0.5 h. LC/MS of the mixture showed 95% of the conversion. Another 0.05 equivalents of NIS was added, and the mixture was stirred for another 30 minutes. LC/MS showed complete conversion. The reaction mixture was added to saturated aqueous NaHCO₃ (50 mL), stirred for 30 minutes and the resulting solids were collected by filtration and dried to obtain 6-ethoxy-3-iodo-1H-pyrrolo[2,3-b]pyridine (XLVIII) (2.134 g, 7.407 mmol, 83.4% yield) as a beige solid. ESIMS found for C₉H₉IN₂O m/z 288.9 (M+H).

The following intermediate was prepared in accordance with the procedure described in the above Scheme 7.

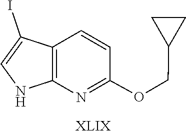 [SMILES: Ic1c[nH]c2nc(OCC3CC3)ccc12]

6-(Cyclopropylmethoxy)-3-iodo-1H-pyrrolo[2,3-b]pyridine (XLIX): Beige solid (396 mg, 1.261 mmol, 93.8% yield). ¹ESIMS found for C₁₁H₁₁IN₂O m/z 314.95 (M+H).

Preparation of intermediate 6-bromo-N-(1-methylpiperidin-4-yl)pyrazin-2-amine (LII) is depicted below in Scheme 8.

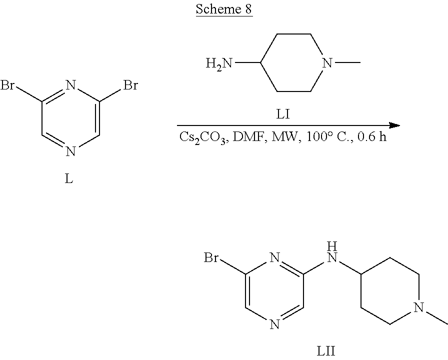

### Step 1

A mixture of 2,6-dibromopyrazine (L) (2.38 g, 9.98 mmol), Cs₂CO₃ (6.51 g, 19.97 mmol) and 4-amino-1-methylpiperidine (LI) (1.25 g, 10.98 mmol) in DMF (15 mL) was heated in a microwave at 100° C. for 0.6 h. The mixture was worked up with EtOAc-brine extraction. the product was purified by silica column (0→20% 7 N NH₃ in MeOH/CHCl₃) to yield 6-bromo-N-(1-methylpiperidin-4-yl)pyrazin-2-amine (LII) (1.06 g, 3.909 mmol, 39.2% yield) as a yellow wax. ESIMS found for C₁₀H₁₅BrN₄ m/z 272.1 (M+H).

Preparation of intermediate 6-bromo-8-fluoro-2-methylimidazo[1,2-a]pyridine (LV) is depicted below in Scheme 9.

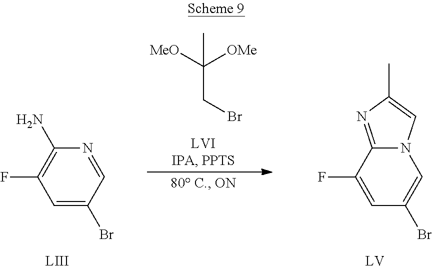

### Step 1

A mixture of 5-bromo-3-fluoropyridin-2-amine (LIII) (2.g, 10.47 mmol), 1-bromo-2,2-dimethoxypropane (LIV) (2.11g, 11.53 mmol) and PPTS (0.26 g, 1.05 mmol) in IPA (25 mL) was heated to 80° C. overnight. The reaction mixture was cooled to room temperature, added to water (200 mL) and stirred for 1 h. The resulting solids were collected and dried under high vacuo to obtain 6-bromo-8-fluoro-2-methylimidazo[1,2-a]pyridine (LV) (2.61 g, 11.40 mmol, 108.8% yield) as beige solid which was used for the next step without further purification. ESIMS found for C₈H₆BrFN₂ m/z 229.0 (M+H).

The following intermediate was prepared in accordance with the procedure described in the above Scheme 9.

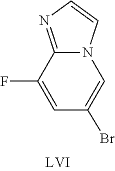 [SMILES: Fc1cc(Br)cn2ccnc12]

6-Bromo-8-fluoroimidazo[1,2-a]pyridine (LVI): Beige solid (909.0 mg, 4.228 mmol, 80.7% yield). ¹ESIMS found for C₇H₄BrFN₂ m/z 214.9 (M+H).

Preparation of intermediate 1-(6-bromoimidazo[1,2-a]pyridin-3-yl)-N,N-dimethylmethanamine (LX) is depicted below in Scheme 10.

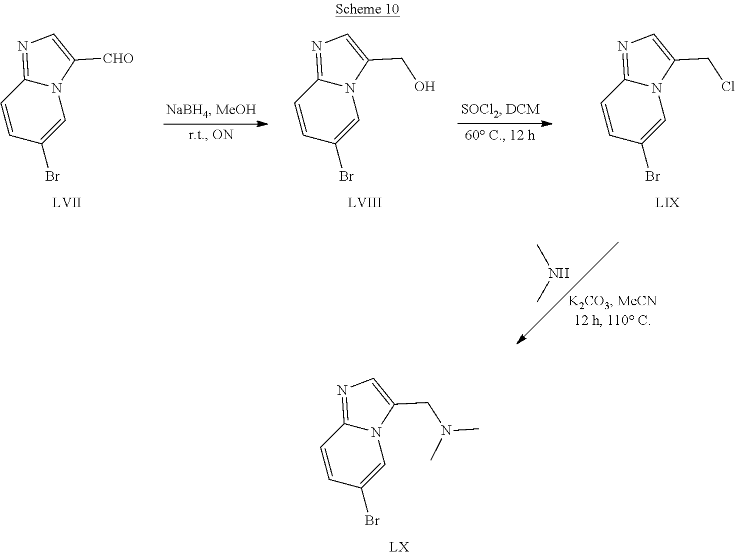

### Step 1

To a solution of NaBH₄ (680 mg, 18.47 mmol) in MeOH (0.67 mL) was added 6-bromoimidazo[1,2-a]pyridine-3-carbaldehyde (LVII) (2.05 g, 9.11 mmol). The reaction mixture was stirred overnight at room temperature. The reaction mixture was concentrated, and the resulting residue was adsorbed on silica gel, purified by column chromatography (0→10% MeOH/CHCl₃). Pure fractions were collected and concentrated, resulting solids were triturated with DCM/hexane, filtered and dried under high vacuum to obtain (6-bromoimidazo[1,2-a]pyridin-3-yl)methanol (LVIII) (1.541 g, 6.787 mmol, 74.5% yield) as a beige solid. ESIMS found for C₈H₇BrN₂O m/z 226.9 (M+H).

### Step 2

A mixture of SOCl₂ (1.5 mL, 20.68 mmol), (6-bromoimidazo[1,2-a]pyridin-3-yl)methanol (LVIII) (1.54 g, 6.78 mmol) in DCM (10 mL) was stirred for 12 h at 60° C. The reaction mixture was concentrated and dried under high vacuum to obtain 6-bromo-3-(chloromethyl)imidazo[1,2-a]pyridine (LIX) (1.7 g, 6.92 mmol, 102.1% yield) as a brown solid used without further for next step. ESIMS found for C₈H₆BrClN₂ m/z 244.9 (M+H).

### Step 3

A mixture of 6-bromo-3-(chloromethyl)imidazo[1,2-a]pyridine (LIX) (600 mg, 2.44 mmol), K₂CO₃ (680 mg, 4.92 mmol) and N-methylmethanamine (0.33 mL, 4.98 mmol) in MeCN (10 mL) was stirred for 12 h at 110° C. The reaction mixture was concentrated resulting residue was adsorbed on silica gel, purified by column chromatography (0→10% MeOH/CHCl₃). Pure fractions were collected and concentrated, and dried under high vacuum to obtain 1-(6-bromoimidazo[1,2-a]pyridin-3-yl)-N,N-dimethylmethanamine (LX) (250 mg, 0.984 mmol, 40.3% yield) as a beige solid. ESIMS found for C₁₀H₁₂BrN₃ m/z 253.95 (M+H).

Preparation of intermediate 5-bromo-3-(1-methyl-1H-pyrazol-4-yl)pyrazolo[1,5-a]pyridine (LXIV) is depicted below in Scheme 11.

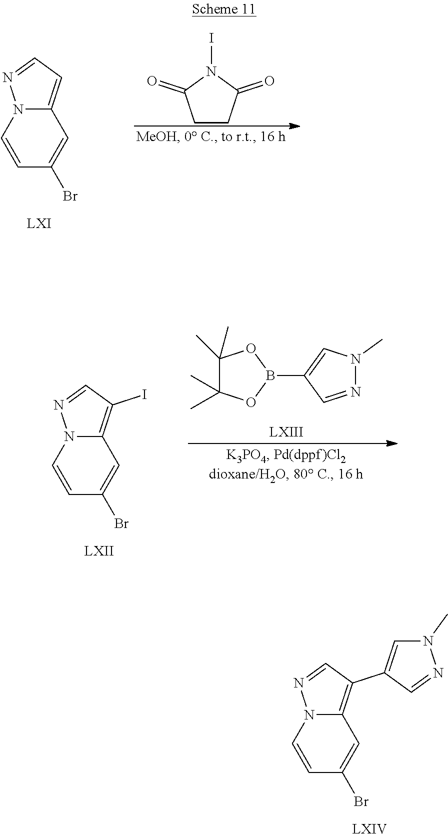

### Step 1

To a solution of 5-bromopyrazolo[1,5-a]pyridine (LXI) (commercially available from Synthonix) (0.36 g, 1.83 mmol) in MeOH (10 mL) was added 1-iodopyrrolidine-2,5-dione (0.41 g, 1.83 mmol) slowly at 0° C. The mixture was stirred at 0° C. for 30 min, then warmed to room temperature and stirred for 16 h. The mixture was concentrated in vacuo and the residue was dissolved in DCM. The organics were washed with an aqueous solution of saturated Na₂S₂O₃. The organics were then concentrated in vacuo to afford 5-bromo-3-iodopyrazolo[1,5-a]pyridine (LXII) (526.0 mg, 1.629 mmol, 89.1% yield) as a white solid. ESIMS found for C₇H₄BrIN₂ m/z 322.8 (⁷⁹BrM+H).

### Step 2

A mixture of 1-methyl-4-(4,4,5,5-tetramethyl-1,3,2-dioxaborolan-2-yl)-1H-pyrazole (LXIII) (230 mg, 1.11 mmol), Pd(dppf)Cl₂ (80 mg, 0.090 mmol) and 5-bromo-3-iodopyrazolo[1,5-a]pyridine (LXII) (300 mg, 0.930 mmol) was taken in 1,4-dioxane (4 mL) and 2 M aqueous solution of K₃PO₄ (1.4 mL, 2.8 mmol) was added. N₂ gas was purged for 5 min. The reaction mixture was heated to 16 h at 80° C. The reaction was then cooled and concentration under vacuum. The residue was purified via column chromatography (0→8% MeOH/CHCl₃) (12 g of silica gel) to produce 5-bromo-3-(1-methyl-1H-pyrazol-4-yl)pyrazolo[1,5-a]pyridine (LXIV) (68 mg, 0.245 mmol, 26.4% yield) as a yellow solid. ESIMS found for C₁₁H₉BrN₄ m/z 276.95 (⁷⁹BrM+H).

The following intermediate was prepared in accordance with the procedure described in the above Scheme 11.

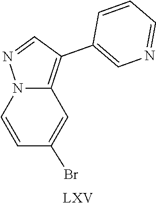 [SMILES: Brc1ccn2ncc(-c3cccnc3)c2c1]

5-Bromo-3-(pyridin-3-yl)pyrazolo[1,5-a]pyridine (LXV): Beige solid (150 mg, 0.547 mmol, 35.3% yield). ESIMS found for C₁₂H₈BrN₃ m/z 273.9 (M+H).

Preparation of intermediate (5-bromopyrazolo[1,5-a]pyridin-3-yl)(4-methylpiperazin-1-yl)methanone (LXVIII) is depicted below in Scheme 12.

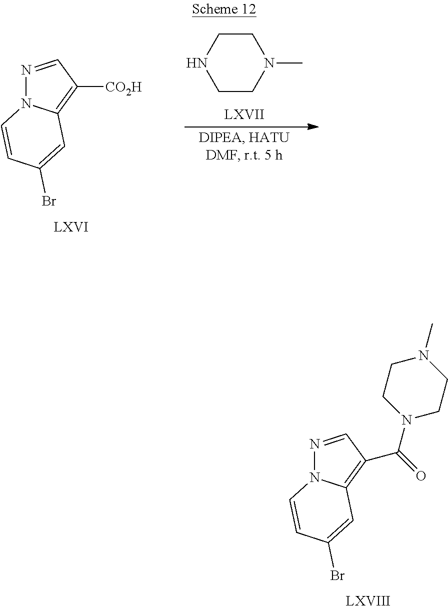

### Step 1

A mixture of 5-bromopyrazolo[1,5-a]pyridine-3-carboxylic acid (LXVI) (Commercially available from Advanced ChemBlocks Inc.) (300 mg, 1.24 mmol), DIPEA (0.44 mL, 2.53 mmol), and HATU (0.47 g, 1.24 mmol) in DMF (4 mL) was stirred at room temperature for 5 min. Then, 1-methylpiperazine (LXVII) (0.28 mL, 2.52 mmol) was added and the reaction mixture was continued at room temperature for 5 h. The solvents were concentrated in vacuo, the residue taken into EtOAc, washed with water, saturated aqueous NaHCO₃, water, and brine. The organic layers were dried over anhydrous Na₂SO₄, then concentrated and under high vacuo to obtain crude (5-bromopyrazolo[1,5-a]pyridin-3-yl)(4-methylpiperazin-1-yl)methanone (LXVIII) (335 mg, 1.037 mmol, 83.3% yield) as a brown gummy solid, which was used in the next step without purification. ESIMS found for C₁₃Hl₅BrN₄O m/z 323.0 (⁷⁹BrM+H).

The following intermediates were prepared in accordance with the procedure described in the above Scheme 12.

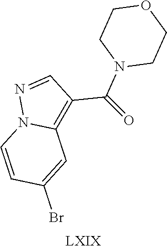 [SMILES: O=C(c1cnn2ccc(Br)cc12)N1CCOCC1]

(5-Bromopyrazolo[1,5-a]pyridin-3-yl)(morpholino)methanone (LXIX): Off-white solid (123.0 mg, 0.397 mmol, 95.6% yield). ESIMS found for C₁₂H₁₂BrN₃O₂ m/z 310.95 (M+H).

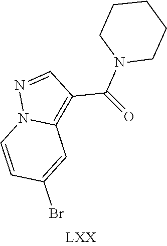 [SMILES: O=C(c1cnn2ccc(Br)cc12)N1CCCCC1]

(5-Bromopyrazolo[1,5-a]pyridin-3-yl)(piperidin-1-yl)methanone (LXX): Light brown solid (121.0 mg, 0.393 mmol, 94.6% yield). ESIMS found for C₁₃H₁₄BrN₃O m/z 308.0 (M+H).

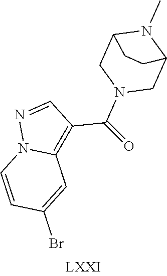 [SMILES: CN1C2CCC1CN(C(=O)c1cnn3ccc(Br)cc13)C2]

(5-Bromopyrazolo[1,5-a]pyridin-3-yl)(8-methyl-3,8-diazabicyclo[3.2.1]octan-3-yl)methanone (LXXI): Beige solid (36 mg, 0.103 mmol, 24.8% yield). ESIMS found for C₁₅H₁₇BrN₄O m/z 349.0 (M+H).

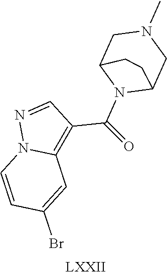 [SMILES: CN1CC2CCC(C1)N2C(=O)c1cnn2ccc(Br)cc12]

(5-Bromopyrazolo[1,5-a]pyridin-3-yl)(3-methyl-3,8-diazabicyclo[3.2.1]octan-8-yl)methanone (LXXII): Beige solid (155 mg, 0.444 mmol, 107.0% yield). ESIMS found for C₁₅H₁₇BrN₄O m/z 349.0 (M+H).

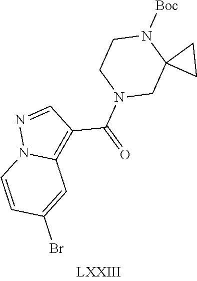 [SMILES: CC(C)(C)OC(=O)N1CCN(C(=O)c2cnn3ccc(Br)cc23)CC12CC2]

tert-Butyl 7-(5-bromopyrazolo[1,5-a]pyridine-3-carbonyl)-4,7-diazaspiro[2.5]octane-4-carboxylate (LXXIII): White foam (320 mg, 0.735 mmol, 88.6% yield). ESIMS found for C₁₉H₂₃BrN₄O₃ m/z 435.0 (M+H).

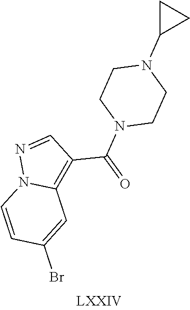 [SMILES: O=C(c1cnn2ccc(Br)cc12)N1CCN(C2CC2)CC1]

(5-Bromopyrazolo[1,5-a]pyridin-3-yl)(4-cyclopropylpiperazin-1-yl)methanone (LXXIV): White solid (145 mg, 0.415 mmol, 100.1% yield). ESIMS found for C₁₅H₁₇BrN₄O m/z 349.1 (M+H).

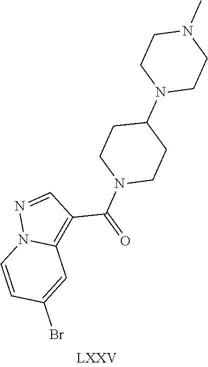 [SMILES: CN1CCN(C2CCN(C(=O)c3cnn4ccc(Br)cc34)CC2)CC1]

(5-Bromopyrazolo[1,5-a]pyridin-3-yl)(4-(4-methylpiperazin-1-yl)piperidin-1-yl)methanone (LXXV): White solid (112 mg, 0.276 mmol, 66.4% yield). ESIMS found for C₁₈H₂₄BrN₅O m/z 406.1 (M+H).

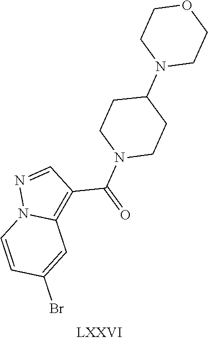 [SMILES: O=C(c1cnn2ccc(Br)cc12)N1CCC(N2CCOCC2)CC1]

(5-Bromopyrazolo[1,5-a]pyridin-3-yl)(4-morpholinopiperidin-1-yl)methanone (LXXVI): White solid (162 mg, 0.412 mmol, 99.2% yield). ESIMS found for C₁₇H₂₁BrN₄O₂ m/z 393.1 (M+H).

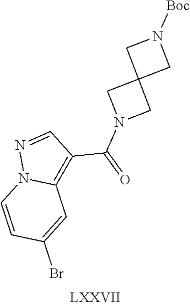 [SMILES: CC(C)(C)OC(=O)N1CC2(C1)CN(C(=O)c1cnn3ccc(Br)cc13)C2]

tert-Butyl 6-(5-bromopyrazolo[1,5-a]pyridine-3-carbonyl)-2,6-diazaspiro[3.3]heptane-2-carboxylate (LXXVII): White solid (323 mg, 0.767 mmol, 92.4% yield). ESIMS found for C₁₈H₂₁BrN₄O₃ m/z 421.1 (M+H).

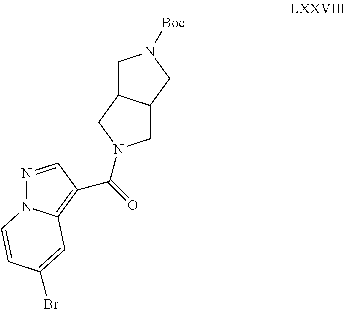 [SMILES: CC(C)(C)OC(=O)N1CC2CN(C(=O)c3cnn4ccc(Br)cc34)CC2C1]

tert-Butyl 5-(5-bromopyrazolo[1,5-a]pyridine-3-carbonyl)hexahydropyrrolo[3,4-c]pyrrole-2(1H)-carboxylate (LXVIII): Clear viscous solid (152 mg, 0.349 mmol, 84.2% yield). ESIMS found for C₁₉H₂₃BrN₄O₃ m/z 379.0 (M+H-^{t}Bu).

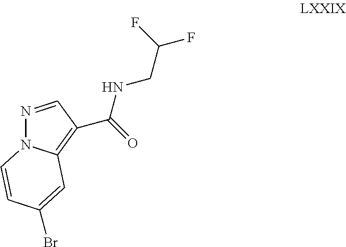 [SMILES: O=C(NCC(F)F)c1cnn2ccc(Br)cc12]

5-Bromo-N-(2,2-difluoroethyl)pyrazolo[1,5-a]pyridine-3-carboxamide (LXXIX): White sold (506 mg, 1.66 mmol, 87.2% yield). ESIMS found for C₁₀H₈BrF₂N₃O m/z 304.0 (M+H).

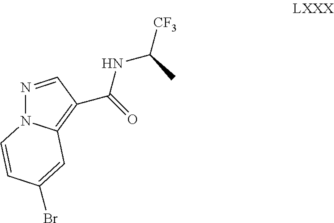 [SMILES: C[C@@H](NC(=O)c1cnn2ccc(Br)cc12)C(F)(F)F]

(R)-5-Bromo-N-(1,1,1-trifluoropropan-2-yl)pyrazolo[1,5-a]pyridine-3-carboxamide (LXXX): White sold (280 mg, 0.833 mmol, 100.4% yield). ESIMS found for C₁₁H₉BrF₃N₃O m/z 336.0 (M+H).

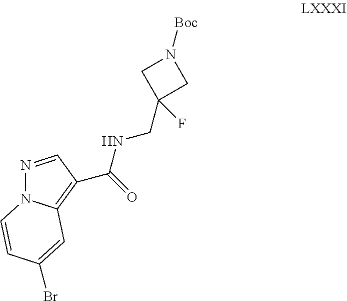 [SMILES: CC(C)(C)OC(=O)N1CC(F)(CNC(=O)c2cnn3ccc(Br)cc23)C1]

tert-Butyl 3-((5-bromopyrazolo[1,5-a]pyridine-3-carboxamido)methyl)-3-fluoroazetidine-1-carboxylate (LXXXI): White sold (244 mg, 0.572 mmol, 69.0% yield). ESIMS found for C₁₇H₂₀BrFN₄O₃ m/z 385.0 (M+H-^{t}Bu+Me).

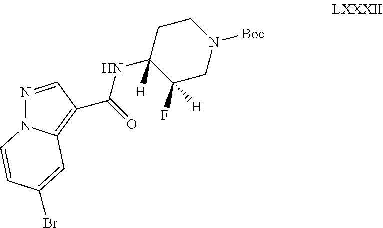 [SMILES: CC(C)(C)OC(=O)N1CC[C@@H](NC(=O)c2cnn3ccc(Br)cc23)[C@H](F)C1]

tert-Butyl (3R,4R)-4-(5-bromopyrazolo[1,5-a]pyridine-3-carboxamido)-3-fluoropiperidine-1-carboxylate (LXXXII): Clear viscous sold (370 mg, 0.839 mmol, 101.1% yield). ESIMS found for C₁₈H₂₂BrFN₄O₃ m/z 385.0 (M+H-^{t}Bu).

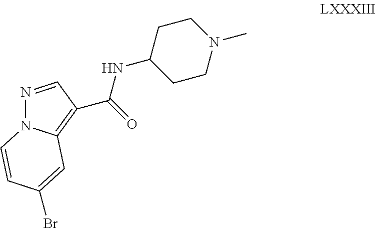 [SMILES: CN1CCC(NC(=O)c2cnn3ccc(Br)cc23)CC1]

5-Bromo-N-(1-methylpiperidin-4-yl)pyrazolo[1,5-a]pyridine-3-carboxamide (LXXXIII): White sold (157.0 mg, 0.466 mmol, 112.2% yield). ESIMS found for C₁₄H₁₇BrN₄O m/z 337.0 (M+H).

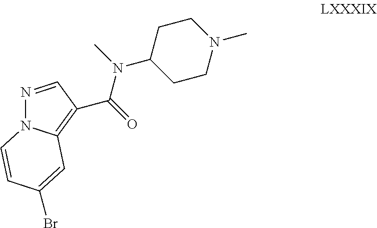 [SMILES: CN1CCC(N(C)C(=O)c2cnn3ccc(Br)cc23)CC1]

5-Bromo-N-methyl-N-(1-methylpiperidin-4-yl)pyrazolo[1,5-a]pyridine-3-carboxamide (LXXXIX): Clear viscous sold (150 mg, 0.427 mmol, 102.9% yield). ESIMS found for C₁₅H₁₉BrN₄O m/z 351.1 (M+H).

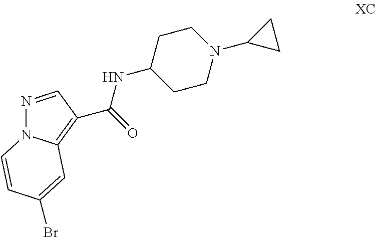 [SMILES: O=C(NC1CCN(C2CC2)CC1)c1cnn2ccc(Br)cc12]

5-Bromo-N-(1-cyclopropylpiperidin-4-yl)pyrazolo[1,5-a]pyridine-3-carboxamide (XC): Beige sold (95 mg, 0.262 mmol, 63.0% yield). ESIMS found for C₁₆H₁₉BrN₄O m/z 363.0 (M+H).

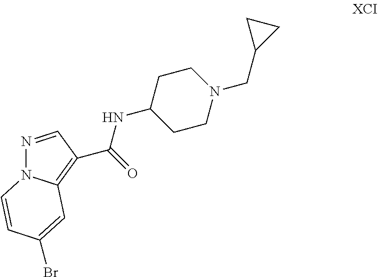 [SMILES: O=C(NC1CCN(CC2CC2)CC1)c1cnn2ccc(Br)cc12]

5-Bromo-N-(1-(cyclopropylmethyl)piperidin-4-yl)pyrazolo[1,5-a]pyridine-3-carboxamide (XCI): White sold (157 mg, 0.416 mmol, 100.3% yield). ESIMS found for C₁₇H₂₁BrN₄O m/z 377.1 (M+H).

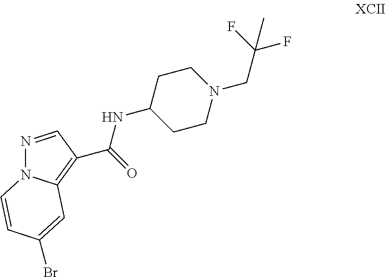 [SMILES: CC(F)(F)CN1CCC(NC(=O)c2cnn3ccc(Br)cc23)CC1]

5-Bromo-N-(1-(2,2-difluoropropyl)piperidin-4-yl)pyrazolo[1,5-a]pyridine-3-carboxamide (XCII): Orange viscous sold (170 mg, 0.424 mmol, 102.1% yield). ESIMS found for C₁₆H₁₉BrF₂N₄O m/z 401.1 (M+H).

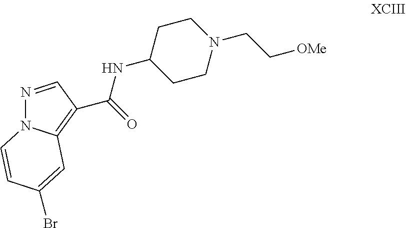 [SMILES: COCCN1CCC(NC(=O)c2cnn3ccc(Br)cc23)CC1]

5-Bromo-N-(1-(2-methoxyethyl)piperidin-4-yl)pyrazolo[1,5-a]pyridine-3-carboxamide (XCIII): White sold (240 mg, 0.630 mmol, 75.9% yield). ESIMS found for C₁₆H₂₁BrN₄O₂ m/z 381.1 (M+H).

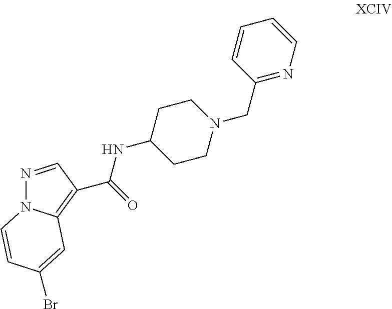 [SMILES: O=C(NC1CCN(Cc2ccccn2)CC1)c1cnn2ccc(Br)cc12]

5-Bromo-N-(1-(pyridin-2-ylmethyl)piperidin-4-yl)pyrazolo[1,5-a]pyridine-3-carboxamide (XCIV): White sold (218 mg, 0.526 mmol, 84.6% yield). ¹ESIMS found for C₁₉H₂₀BrN₅O m/z 414.1 (M+H).

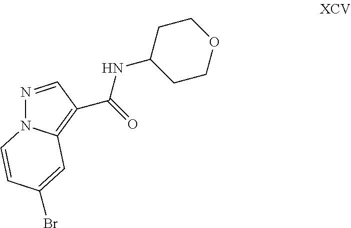 [SMILES: O=C(NC1CCOCC1)c1cnn2ccc(Br)cc12]

5-Bromo-N-(tetrahydro-2H-pyran-4-yl)pyrazolo[1,5-a]pyridine-3-carboxamide (XCV): White solid (85 mg, 0.262 mmol, 63.2% yield). ESIMS found for C₁₃H₁₄BrN₃O₂ m/z 324.0 (M+H).

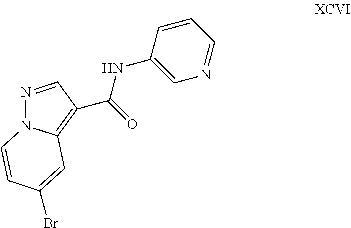 [SMILES: O=C(Nc1cccnc1)c1cnn2ccc(Br)cc12]

5-Bromo-N-(pyridin-3-yl)pyrazolo[1,5-a]pyridine-3-carboxamide (XCVI): Light brown solid (86.0 mg, 0.271 mmol, 65.4% yield). ESIMS found for C₁₃H₉BrN₄O m/z 316.9 (M+H).

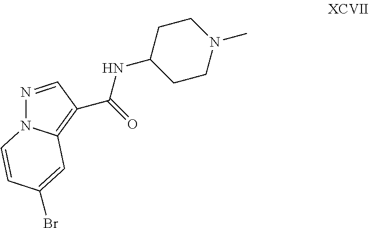 [SMILES: CN1CCC(NC(=O)c2cnn3ccc(Br)cc23)CC1]

6-Bromo-N-(1-methylpiperidin-4-yl)imidazo[1,2-a]pyridine-3-carboxamide (XCVII): Beige solid (349 g, 0.035 mmol, 99.8% yield). ESIMS found for C₁₄H₁₇BrN₄O m/z 337.0 (M+H).

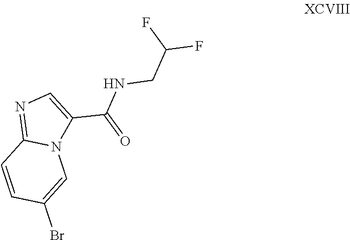 [SMILES: O=C(NCC(F)F)c1cnc2ccc(Br)cn12]

6-Bromo-N-(2,2-difluoroethyl)imidazo[1,2-a]pyridine-3-carboxamide (XCVIII): Light yellow solid (240 mg, 0.789 mmol, 95.1% yield). ESIMS found for C₁₀H₈BrF₂N₃O m/z 304.0 (M+H).

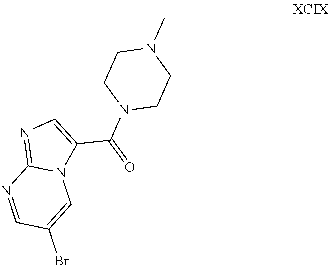 [SMILES: CN1CCN(C(=O)c2cnc3ncc(Br)cn23)CC1]

(6-Bromoimidazo[1,2-a]pyrimidin-3-yl)(4-methylpiperazin-1-yl)methanone (XCIX): Gummy white solid (159 mg, 0.491 mmol, 59.4% yield). ESIMS found for C₁₂H₁₄BrN₅O m/z 324.0 (M+H).

Preparation of intermediate imidazo[1,2-a]pyrimidin-6-ylboronic acid (CII) is depicted below in Scheme 13.

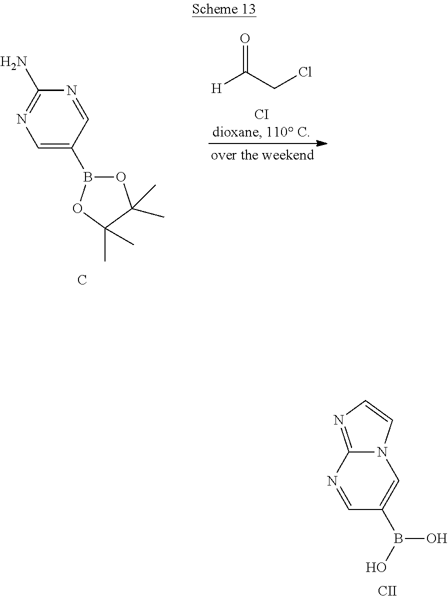

### Step 1

A mixture of 5-(4,4,5,5-tetramethyl-1,3,2-dioxaborolan-2-yl)pyrimidin-2-amine (C) (1 g, 4.52 mmol) and chloroacetaldehyde (CI) (0.92 mL, 5.39 mmol) was dissolved in 1,4-dioxane (20 mL) and heated to 110° C. over the weekend. The reaction mixture was cooled, and the solids were collected by filtration and dried under high vacuo to obtain imidazo[1,2-a]pyrimidin-6-ylboronic acid (CII) (650 mg, 3.989 mmol, 88.2% yield) as a brown solid which was used for next step without purification. ESIMS found for C₆H₆BN₃O₂ m/z 164.1 (M+H).

Preparation of intermediate 6-bromo-1-isopropyl-2-methyl-1H-imidazo[4,5-b]pyridine (CVII) is depicted below in Scheme 14.

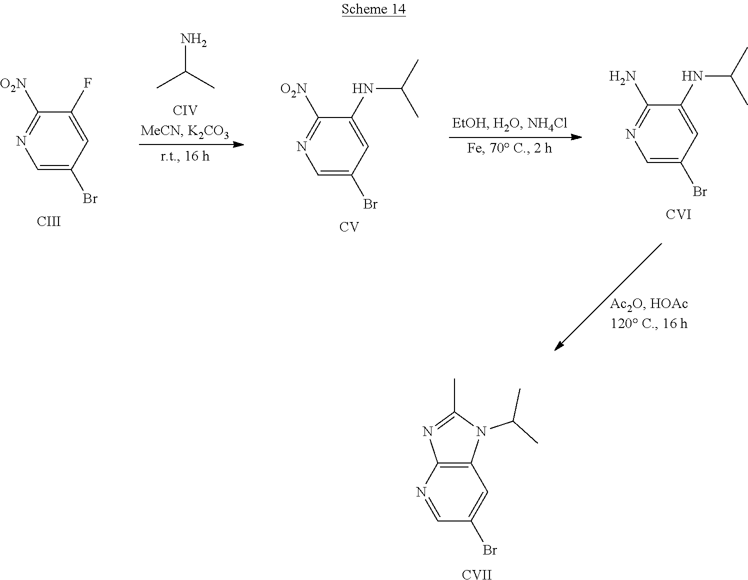

### Step 1

A mixture of 2-aminopropane (CIV) (0.86 mL, 9.96 mmol), 5-bromo-3-fluoro-2-nitropyridine (CIII) (2 g, 9.05 mmol) and K₂CO₃ (2.5 g, 18.1 mmol) in MeCN (40 mL) was stirred at room temperature for 16 h. The reaction mixture was added to water (200 mL), stirred for 1 h and the resulting solids were collected by filtration and dried under high vacuo to obtain 5-bromo-N-isopropyl-2-nitropyridin-3-amine (CV) (2.36 g, 9.074 mmol, 100.3% yield) as a yellow solid which was used for next step without purification. ESIMS found for C₈H₁₀BrN₃O₂ m/z 260.0 (M+H).

### Step 2

A mixture of 5-bromo-N-isopropyl-2-nitropyridin-3-amine (CV) (2.35 g, 9.04 mmol) Fe (5.91 g, 90.35 mmol) and NH₄Cl (7.25 g, 135.53 mmol) was taken in a mixture of EtOH (30 mL) and water (10 mL) and the mixture was heated to 70° C. for 2 h. The reaction mixture was cooled, filtered through Celite®, filtrates were taken into EtOAc, washed with water then brine, dried over anhydrous Na₂SO₄, filtered, and concentrated under reduced pressure to obtain 5-bromo-N³-isopropylpyridine-2,3-diamine (CVI) (2.2 g, 9.561 mmol, 105.8% yield) as a dark brown solid which was used for next step without purification. ESIMS found for C₈H₁₂BrN₃ m/z 230.05 (M+H).

### Step 3

A solution of 5-bromo-N³-isopropylpyridine-2,3-diamine (CVI) (2.08 g, 9.04 mmol) and Ac₂O (1.05 mL, 10.84 mmol) in HOAc (20 mL) was heated to 120° C. for 16 h. The reaction mixture was concentrated, the residue partitioned between EtOAc/1 N NaOH, organics separated, washed with water and brine. The organics were dried over anhydrous Na₂SO₄, solvents concentrated and dried under high vacuo to give 6-bromo-1-isopropyl-2-methyl-1H-imidazo[4,5-b]pyridine (CVII) (1.57 g, 6.178 mmol, 68.3% yield) as a dark brown solid which was used for next step without purification. ESIMS found for C₁₀H₁₂BrN₃ m/z 254.0 (M+H).

Preparation of intermediate 6-bromo-4-fluoro-2-methyl-1-(1-methylpiperidin-4-yl)-1H-benzo[d]imidazole (CXIII) is depicted below in Scheme 15.

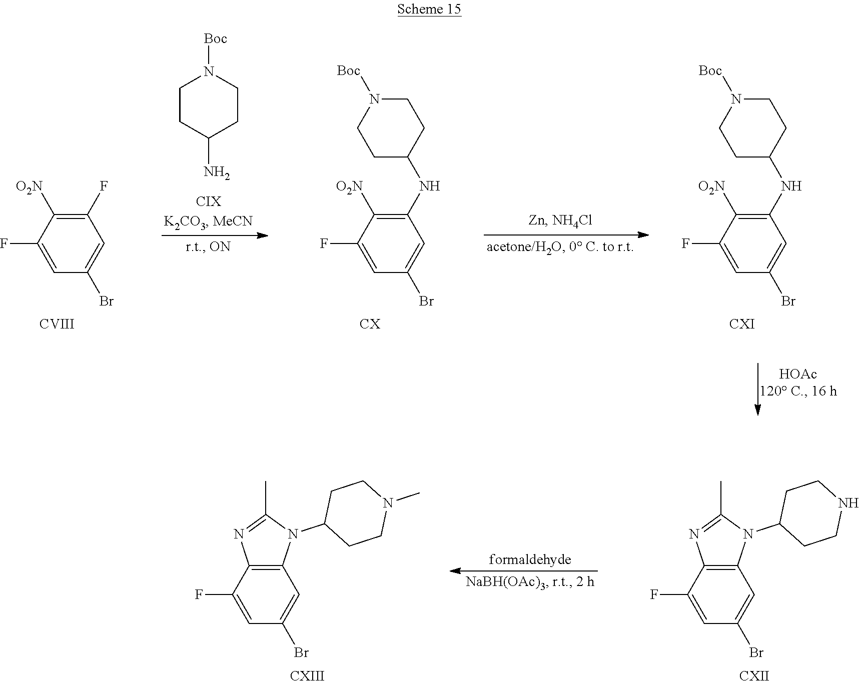

### Step 1

A mixture of tert-butyl 4-aminopiperidine-1-carboxylate (CIX) (commercially available from Combi-Blocks Inc.) (1.68 g, 8.4 mmol), 5-bromo-1,3-difluoro-2-nitrobenzene (CVIII) (commercially available from Combi-Blocks Inc.) (2 g, 8.4 mmol) and K₂CO₃ (2.32 g, 16.81 mmol) in MeCN (40 mL) was stirred at room temperature over the weekend. The solvents were concentrated, and the residue partitioned between EtOAc/water. The organics were separated and washed with water and brine solution. The organics were dried over anhydrous Na₂SO₄, solvents removed in vacuo and the crude was purified by ISCO (0→30% EtOAc/hexanes) to obtain tert-butyl 4-((5-bromo-3-fluoro-2-nitrophenyl)amino) piperidine-1-carboxylate (CX) (2.564 g, 6.130 mmol, 72.9% yield) as a yellow solid. ESIMS found for C₁₆H₂₁BrFN₃O₄ m/z 362.05 (M+H-tBu).

### Step 2

To a solution of tert-butyl 4-((5-bromo-3-fluoro-2-nitrophenyl)amino) piperidine-1-carboxylate (CX) (2.6 g, 6.22 mmol) and NH₄Cl (4.99 g, 93.24 mmol) in a mixture of acetone (75 mL)and water (15 mL) was added zinc powder (4.06 g, 62.16 mmol) (three equal portions over 5 minutes) at 0° C. The mixture was stirred until reaction had warmed to 23° C. The mixture was filtered through Celite® and the solvents were concentrated under high vacuum. The mixture was re-dissolved in EtOAc and filtered a second time through Celite® and the filtrates were washed with water and brine, dried over anhydrous Na₂SO₄, filtered, and concentrated under reduced pressure to obtain tert-butyl 4-((2-amino-5-bromo-3-fluorophenyl)amino)piperidine-1-carboxylate (CXI) (2.41 g, 6.207 mmol, 99.8% yield) as a light brown solid. The resultant residue was used in the next reaction without further purification. ESIMS found for C₁₆H₂₃BrFN₃O₂ m/z 332.0 (M+H-tBu).

### Step 3

A solution of tert-butyl 4-((2-amino-5-bromo-3-fluorophenyl)amino) piperidine-1-carboxylate (CXI) (900 mg, 2.32 mmol) in HOAc (12 mL) was heated to 120° C. for 16 h. The reaction mixture was concentrated, the residue treated with 7 N NH₃/MeOH, absorbed on silica and was purified by ISCO (0→8% 7 N NH₃ in MeOH/CHCl₃) to obtain 6-bromo-4-fluoro-2-methyl-1-(piperidin-4-yl)-1H-benzo[d]imidazole (CXII) (510 mg, 1.634 mmol, 70.5% yield) as a brown solid. ESIMS found for C₁₃H₁₅BrFN₃ m/z 312.1 (M+H).

### Step 4

To a stirred solution of 6-bromo-4-fluoro-2-methyl-1-(piperidin-4-yl)-1H-benzo[d]imidazole (CXII) (510 mg, 1.63 mmol) in MeOH (8 mL) was added formaldehyde (0.41 mL, 2.04 mmol). After 15 min, NaBH(OAc)₃ (520 mg, 2.45 mmol) was added, and the mixture was stirred at room temperature for 2 h. LC-MS of the mixture showed completion of the reaction. The solvents were concentrated, the residue taken up in chloroform, washed with 1 N NaOH, water and brine solution. The organics were dried over anhydrous Na₂SO₄, solvents removed and dried under high vacuo to obtain crude 6-bromo-4-fluoro-2-methyl-1-(1-methylpiperidin-4-yl)-1H-benzo[d]imidazole (CXIII) (440 mg, 1.349 mmol, 82.5% yield) as a light brown solid which was used in the next step without purification. ESIMS found for C₁₄H₁₇BrFN₃ m/z 326. (M+H).

Preparation of intermediate 6′-bromospiro[cyclohexane-1,1′-isoindolin]-3′-one (CXIX) is depicted below in Scheme 16.

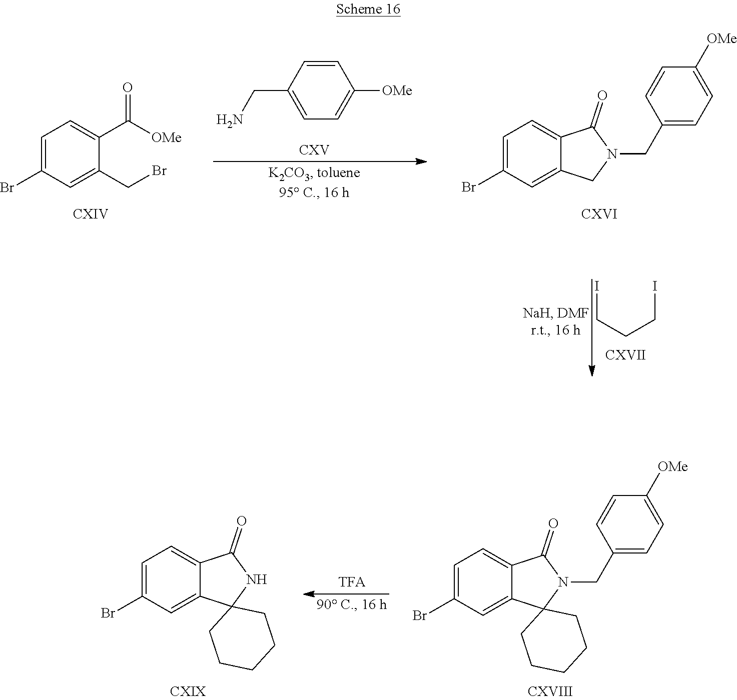

### Step 1

A mixture of methyl 4-bromo-2-(bromomethyl)benzoate (CXIV) (5 g, 16.24 mmol), 4-methoxybenzylamine (CXV) (4.25 mL, 32.75 mmol) and K₂CO₃ (9.02 g, 65.26 mmol) in toluene (100 mL) was heated to 95° C. for 16 h. The reaction mixture was cooled to room temperature, added to water (200 mL), and extracted with EtOAc. The insoluble solids were collected by filtration (product not soluble, 1.25 g), organic layer separated, washed with brine, dried over anhydrous Na₂SO₄, and the solvents were concentrated in vacuo. The residue was suspended in EtOAc (50 mL), sonicated, the solids collected by filtration, washed with diethyl ether, and dried under high vacuo to obtain 5-bromo-2-[(4-methoxyphenyl)methyl]-3H-isoindol-1-one (CXVI) (3.86 g, 11.62 mmol, 71.6% yield) as a white solid. ESIMS found for C₁₆H₁₄BrNO₂ m/z 332.0 (M+H).

### Step 2

To a stirred suspension of 5-bromo-2-[(4-methoxyphenyl)methyl]-3H-isoindol-1-one (CXVI) (1.0 g, 3.01 mmol) in DMF (15 mL) was added NaH (301 mg, 7.53 mmol) and the mixture was stirred for 20 min. 1,5-Diiodopentane (CXVII) (0.58 mL, 3.9 mmol) was then added, and the mixture was stirred at room temperature for 16 h. The reaction mixture was added to water (150 mL), extracted with EtOAc, washed with brine, dried over anhydrous Na₂SO₄, solvents concentrated, and the crude was purified by ISCO (0→60% EtOAc/hexanes) to obtain 5′-bromo-2′-[(4-methoxyphenyl)methyl]spiro[cyclohexane-1,3′-isoindole]-1′-one (CXVIII) (0.37 g, 0.92 mmol, 30.7% yield) as a colorless thick syrup. ESIMS found for C₂₁H₂₂BrNO₂ m/z 400.1 (M+H).

### Step 3

A solution of 5′-bromo-2′-[(4-methoxyphenyl)methyl]spiro[cyclohexane-1,3′-isoindole]-1′-one (CXVIII) (0.37 g, 0.92 mmol) in TFA (4 mL) was heated to 90° C. for 16 h The solvents were concentrated, treated with 7 N NH₃/MeOH and the residue was purified by ISCO (20→100% 10% 7 N NH₃ MeOH in CHCl₃/CHCl₃) to obtain 6′-bromospiro[cyclohexane-1,1′-isoindolin]-3′-one (CXIX) (135 mg, 0.482 mmol, 52.1% yield) as a beige solid. ESIMS found for C₁₃H₁₄BrNO m/z 280.0 (M+H).

Preparation of intermediate 8-bromo-4-methyl-3,4-dihydrobenzo[f][1,4] oxazepin-5(2H)-one (CXXIII) is depicted below in Scheme 17.

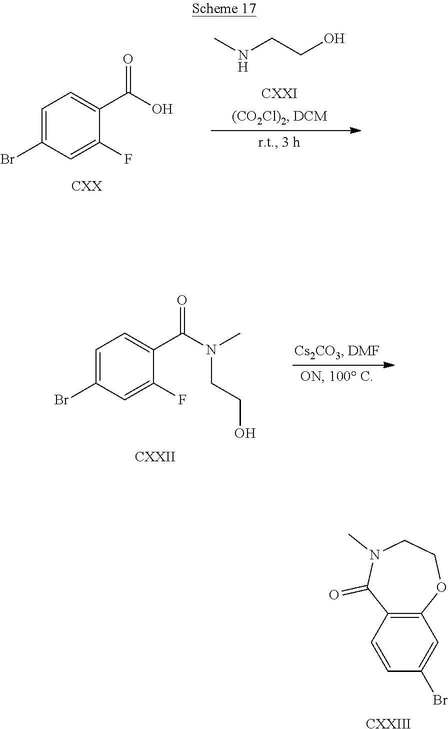

### Step 1

Oxalyl chloride (348 mg, 2.74 mmol) was added to the cold solution of 4-bromo-2-fluorobenzoic acid (CXX) (500 mg, 2.28 mmol) in DCM (6 mL). Two drops of DMF was then added and allowed to stir for about 30 min. Solvents were removed and kept under vacuum for about 30 min. Redissolved in DCM (6 mL) and added 2-(methylamino)ethanol (CXXI) (39 mg, 5.25 mmol). The reaction was stirred at room temperature for 3 h. The reaction was concentrated and purified on column (20→100% EtOAc/hexanes), pure fractions were combined and concentrated to yield 4-bromo-2-fluoro-N-(2-hydroxyethyl)-N-methylbenzamide (CXXII) (525 mg, 1.902 mmol, 83.3% yield) as a white solid. ESIMS found for C₁₀H₁₁BrFNO₂ m/z 276.0 (M+H).

### Step 2

To a solution of 4-bromo-2-fluoro-N-(2-hydroxyethyl)-N-methylbenzamide (CXXII) (525 mg, 1.9 mmol) dissolved in DMF (8 mL) was added Cs₂CO₃ (1.239 g, 3.8 mmol). The reaction was stirred at 100° C. overnight. LC/MS suggest almost 80% conversion. Water was added, filtered, and the solid purified on column (0→70% EtOAc/hexanes) to give 8-bromo-4-methyl-3,4-dihydrobenzo[f][1,4]oxazepin-5(2H)-one (CXXIII) (254 mg, 0.992 mmol, 52.2% yield) as a white solid. ESIMS found for C₁₀H₁₀BrNO₂ m/z 256.0 (M+H).

Preparation of intermediate 6-bromo-4-(4-methylpiperazin-1-yl)quinazoline (CXXV) is depicted below in Scheme 18.

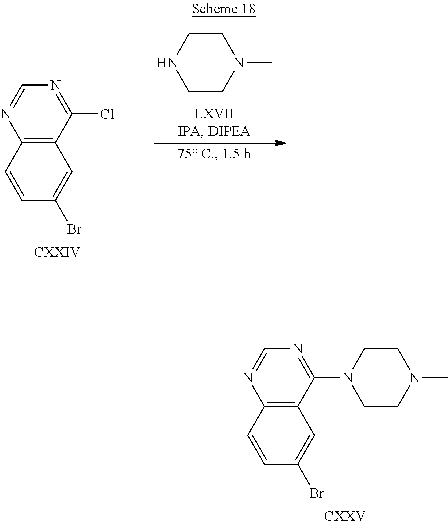

### Step 1

To a stirred suspension of 6-bromo-4-chloroquinazoline (CXXIV) (commercially available from Enamine Ltd) (0.5 g, 2.05 mmol) in IPA (5 mL) was added DIPEA (0.72 mL, 4.13 mmol) and 1-methylpiperazine (LXVII) (632.1 mg, 6.310 mmol). The mixture becomes clear solution in few minutes and was heated to 75° C. for 1.5 h. The solvents were concentrated, the residue partitioned between EtOAc/water, organics separated, washed with brine, dried over anhydrous Na₂SO₄, solvents concentrated and dried under high vacuo to obtain crude 6-bromo-4-(4-methylpiperazin-1-yl)quinazoline (CXXV) (645.0 mg, 2.100 mmol, 102.3% yield) as a thick brown gum which was used for next the step without purification. ESIMS found for C₁₃H₁₅BrN₄ m/z 307.0 (M+H).

Preparation of intermediate 7-bromo-2-((1-methylpiperidin-4-yl)oxy)quinoxaline (CXXVIII) is depicted below in Scheme 19.

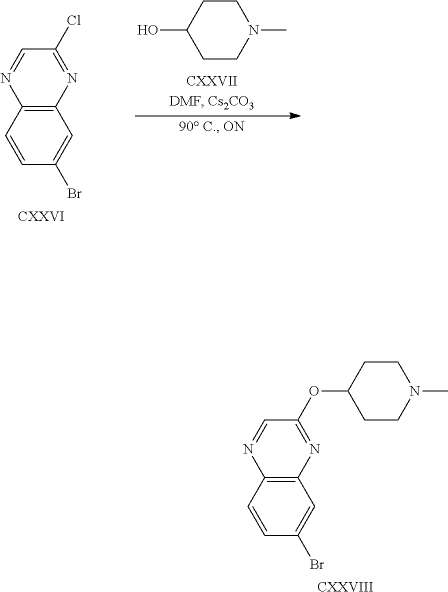

### Scheme 19

### Step 1

A mixture of 1-methyl-4-piperidinol (CXXVII) (0.36 g, 3.13 mmol), Cs₂CO₃ (1.34 g, 4.11 mmol) and 7-bromo-2-chloro-quinoxaline (CXXVI) (commercially available from Enamine Ltd) (0.5 g, 2.05 mmol) in DMF (6 mL) was heated to 90° C. overnight. The solvents were concentrated, and the residue partitioned between EtOAc/water. The organic layer was separated, washed with brine, dried over anhydrous Na₂SO₄, concentrated. The residue was purified by ISCO (0→50% 10% 7 N NH₃ in MeOH in CHCl₃/CHCl₃). The pure fractions were concentrated, dried under high vacuo to obtain 7-bromo-2-(1-methylpiperidin-4-yl)oxyquinoxaline (CXXVIII) (293.0 mg, 0.909 mmol, 44.3% yield) as a beige solid. ESIMS found for C₁₄H₁₆BrN₃O m/z 322.0 (M+H).

The following intermediate was prepared in accordance with the procedure described in the above Scheme 19.

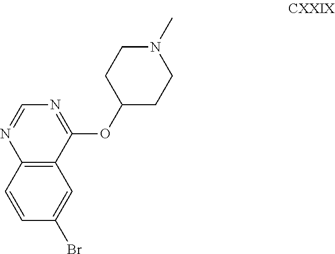 [SMILES: CN1CCC(Oc2ncnc3ccc(Br)cc23)CC1]

6-Bromo-4-((1-methylpiperidin-4-yl)oxy)quinazoline (CXXIX): Off-white solid (293 mg, 0.909 mmol, 44.3% yield). ESIMS found for C₁₄H₁₆BrN₃O m/z 322.0 (M+H).

Preparation of intermediate 7-bromo-N-(1-methylpiperidin-4-yl)quinoxalin-2-amine (CXXXI) is depicted below in Scheme 20.

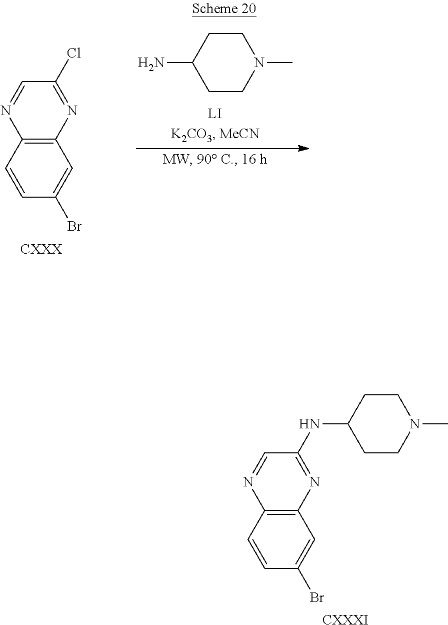

### Step 1

To a microwave vial was added 7-bromo-2-chloro-quinoxaline (CXXX) (commercially available from Combi-Blocks Inc.) (100 mg, 0.41 mmol), 4-amino-1-methylpiperidine (LI) (94 mg, 0.82 mmol), K₂CO₃ (170 mg, 1.23 mmol) and MeCN (2 mL). The reaction mixture was irradiated at 90° C. for 16 h. The reaction was cooled, filtered, and concentrated under vacuum. The crude product was dissolved in DCM, washed with water, brine, dried over anhydrous MgSO₄, and filtered to produce 7-bromo-N-(1-methylpiperidin-4-yl)quinoxalin-2-amine (CXXXI) (95 mg, 0.296 mmol, 72.0% yield) as a beige solid. Carried onto the next step without further purification. ESIMS found for C₁₄H₁₇BrN₄ m/z 321.0 (M+H).

The following intermediates were prepared in accordance with the procedure described in the above Scheme 20.

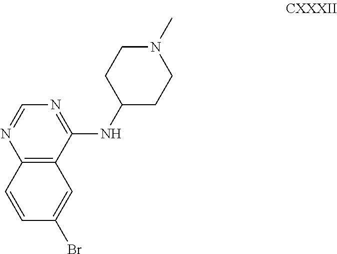 [SMILES: CN1CCC(Nc2ncnc3ccc(Br)cc23)CC1]

6-Bromo-N-(1-methylpiperidin-4-yl)quinazolin-4-amine (CXXXII): Tan solid (105 mg, 0.327 mmol, 79.6% yield). ESIMS found for C₁₄H₁₇BrN₄ m/z 321.0 (M+H).

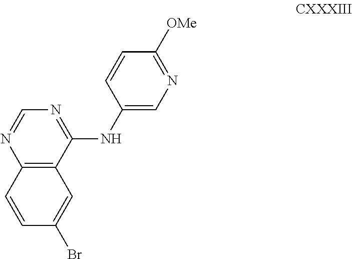 [SMILES: COc1ccc(Nc2ncnc3ccc(Br)cc23)cn1]

6-Bromo-N-(6-methoxypyridin-3-yl)quinazolin-4-amine (CXXXIII): Yellow solid (109 mg, 0.329 mmol, 80.1% yield). ESIMS found for C₁₄H₁₁BrN₄O m/z 330.9 (M+H).

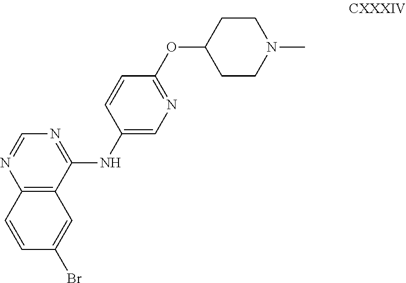 [SMILES: CN1CCC(Oc2ccc(Nc3ncnc4ccc(Br)cc34)cn2)CC1]

6-Bromo-N-(6-((1-methylpiperidin-4-yl)oxy)pyridin-3-yl)quinazolin-4-amine (CXXXIV): Light brown solid (308 mg, 0.743 mmol, 90.5% yield). ESIMS found for C₁₉H₂₀BrN₅O m/z 414.0 (M+H).

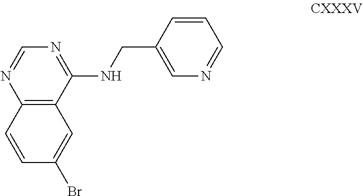 [SMILES: Brc1ccc2ncnc(NCc3cccnc3)c2c1]

6-Bromo-N-(pyridin-3-ylmethyl)quinazolin-4-amine (CXXXV): Yellow solid (587 mg, 1.863 mmol, 90.7% yield). ESIMS found for C₁₄H₁₁BrN₄ m/z 314.95 (M+H).

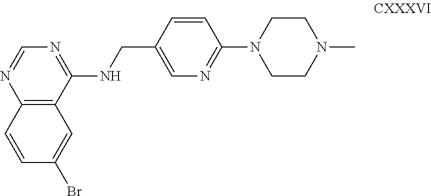 [SMILES: CN1CCN(c2ccc(CNc3ncnc4ccc(Br)cc34)cn2)CC1]

6-Bromo-N-((6-(4-methylpiperazin-1-yl)pyridin-3-yl)methyl)quinazolin-4-amine (CXXXVI): White solid (270 mg, 0.653 mmol, 79.5% yield). ESIMS found for C₁₉H₂₁BrN₆ m/z 413.0 (M+H).

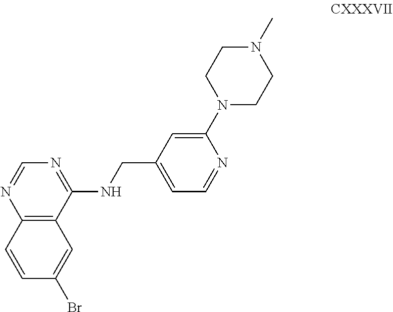 [SMILES: CN1CCN(c2cc(CNc3ncnc4ccc(Br)cc34)ccn2)CC1]

6-Bromo-N-((2-(4-methylpiperazin-1-yl)pyridin-4-yl)methyl)quinazolin-4-amine (CXXXVII): White solid (455 mg, 1.101 mmol, 89.4% yield). ESIMS found for C₁₉H₂₁BrN₆ m/z 413.0 (M+H).

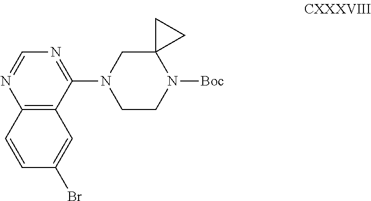 [SMILES: CC(C)(C)OC(=O)N1CCN(c2ncnc3ccc(Br)cc23)CC12CC2]

tert-Butyl 7-(6-bromoquinazolin-4-yl)-4,7-diazaspiro[2.5]octane-4-carboxylate (CXXXVIII): Yellow solid (800 mg, 1.908 mmol, 92.9% yield). ESIMS found for C₁₉H₂₃BrN₄O₂ m/z 419.0 (M+H).

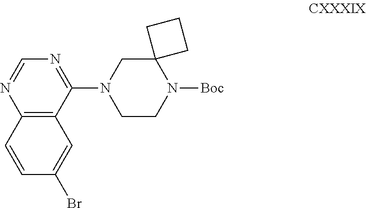 [SMILES: CC(C)(C)OC(=O)N1CCN(c2ncnc3ccc(Br)cc23)CC12CCC2]

tert-Butyl 8-(6-bromoquinazolin-4-yl)-5,8-diazaspiro[3.5]nonane-5-carboxylate (CXXXIX): Beige solid (500 mg, 1.353 mmol, 54.9% yield). ESIMS found for C₂₀H₂₅BrN₄O₂ m/z 333.0 (M+H-Boc).

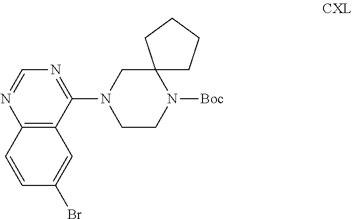 [SMILES: CC(C)(C)OC(=O)N1CCN(c2ncnc3ccc(Br)cc23)CC12CCCC2]

tert-Butyl 9-(6-bromoquinazolin-4-yl)-6,9-diazaspiro[4.5]decane-6-carboxylate (CXL): Beige solid (750 mg, 2.160 mmol, 105.2% yield). ESIMS found for C₂₁H₂₇BrN₄O₂ m/z 347.0 (M+H-Boc).

Preparation of intermediate tert-butyl 7-(6-bromoquinoline-4-carbonyl)-4,7-diazaspiro[2.5]octane-4-carboxylate (CXLIII) is depicted below in Scheme 21.

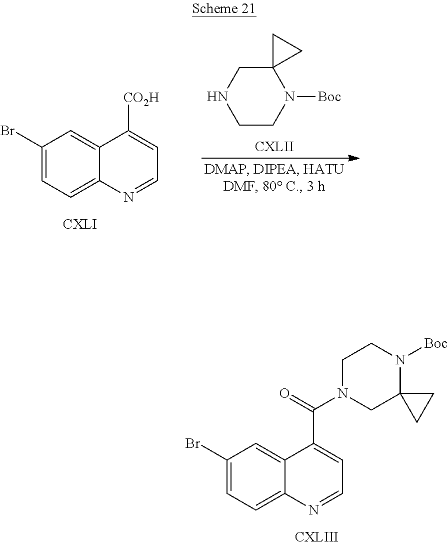 [SMILES: O=C(O)c1ccnc2ccc(Br)cc12 ; CC(C)(C)OC(=O)N1CCNCC12CC2 ; CC(C)(C)OC(=O)N1CCN(C(=O)c2ccnc3ccc(Br)cc23)CC12CC2]

### Step 1

To a suspension of tert-butyl 4,7-diazaspiro[2.5]octane-4-carboxylate (CXLII) (commercially available from Combi-Blocks Inc.) (620 mg, 2.92 mmol), 6-bromoquinoline-4-carboxylic acid (CXLI) (commercially available from Combi-Blocks Inc.) (0.7 g, 2.78 mmol), DMAP (150 mg, 1.23 mmol) and HATU (1.2 g, 3.16 mmol) in DMF (7 mL) was added DIPEA (1 mL, 5.74 mmol). The resulting mixture was stirred at 80° C. for 3 h. The mixture was cooled to room temperature and added into the water. The resulting solid was filtered and dried under high vacuo to afford tert-butyl 7-(6-bromoquinoline-4-carbonyl)-4,7-diazaspiro[2.5]octane-4-carboxylate (CXLIII) (1.1 g, 2.465 mmol, 88.7% yield) as a brown solid. ESIMS found for C₂₁H₂₄BrN₃O₃ m/z 446.0 (M+H).

The following intermediates were prepared in accordance with the procedure described in the above Scheme 21.

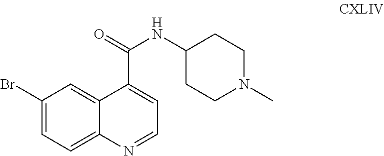 [SMILES: CN1CCC(NC(=O)c2ccnc3ccc(Br)cc23)CC1]

6-Bromo-N-(1-methylpiperidin-4-yl)quinoline-4-carboxamide (CXLIV): White solid (210 mg, 0.603 mmol, 50.7% yield). ESIMS found for C₁₆H₁₈BrN₃O m/z 348.0 (M+H).

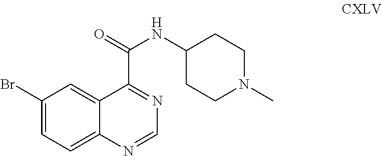 [SMILES: CN1CCC(NC(=O)c2ncnc3ccc(Br)cc23)CC1]

6-Bromo-N-(1-methylpiperidin-4-yl)quinazoline-4-carboxamide (CXLV): Brown solid (300 mg, 0.859 mmol, 72.5% yield). ESIMS found for C₁₅H₁₇BrN₄O m/z 349.0 (M+H).

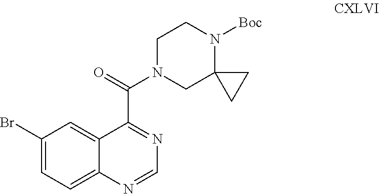 [SMILES: CC(C)(C)OC(=O)N1CCN(C(=O)c2ncnc3ccc(Br)cc23)CC12CC2]

tert-Butyl 7-(6-bromoquinazoline-4-carbonyl)-4,7-diazaspiro[2.5]octane-4-carboxylate (CXLVI): Brown solid (279 mg, 0.604 mmol, 76.4% yield). ESIMS found for C₂₀H₂₃BrN₄O₃ m/z 447.0 (M+H).

### Example 1

Preparation of 2-(1H-Pyrrolo[2,3-b]pyridin-3-yl)-1H-imidazo[4,5-c]pyridine (7) is depicted below in Scheme 22.

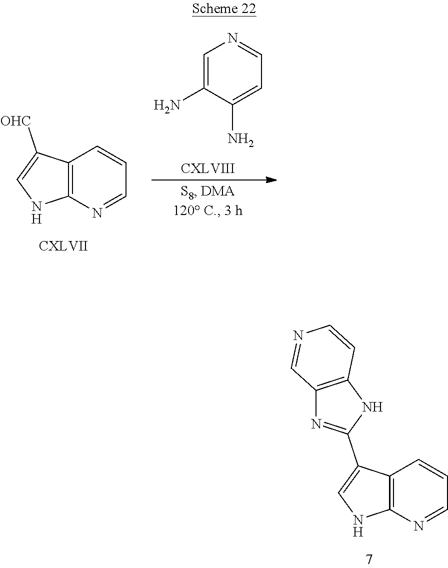 [SMILES: O=Cc1c[nH]c2ncccc12 ; Nc1ccncc1N ; c1cnc2[nH]cc(-c3nc4cnccc4[nH]3)c2c1]

### Step 1

A mixture of 1H-pyrrolo[2,3-b]pyridine-3-carbaldehyde (CXLVII) (commercially available from Combi-Blocks Inc.) (100 mg, 0.68 mmol), sulfur (44 mg, 1.37 mmol) and 3,4-diaminopyridine (CXLVIII) (75 mg, 0.69 mmol) in DMA (2 mL) was heated to 120° C. for 3 h. The solvent was evaporated the crude residue was absorbed on silica gel and purified by column chromatography (10% 7 N NH₃ MeOH/CHCl₃) to obtain 2-(1H-Pyrrolo[2,3-b]pyridin-3-yl)-1H-imidazo[4,5-c]pyridine (7) (20 mg, 0.085 mmol, 12.4% yield) as a beige solid. ¹H NMR (499 MHz, DMSO-d₆) δ ppm 7.29 (1H, dd, J=7.82, 4.53 Hz), 7.55 (1H, br s), 8.28 (1H, d, J=5.49 Hz), 8.33 (1H, br s), 8.36 (1H, dd, J=4.53, 1.51 Hz), 8.79 (1H, dd, J=7.96, 1.37 Hz), 8.89 (1H, br s), 12.30 (1H, br s), 12.99 (1H, br s); ESIMS found for C₁₃H₉N₅ m/z 236.1 (M+1).

### Example 2

Preparation of 6-(4-(1-methyl-1H-pyrazol-4-yl)-1H-pyrrolo[2,3-b]pyridin-3-yl) quinoline (186) is depicted below in Scheme 23.

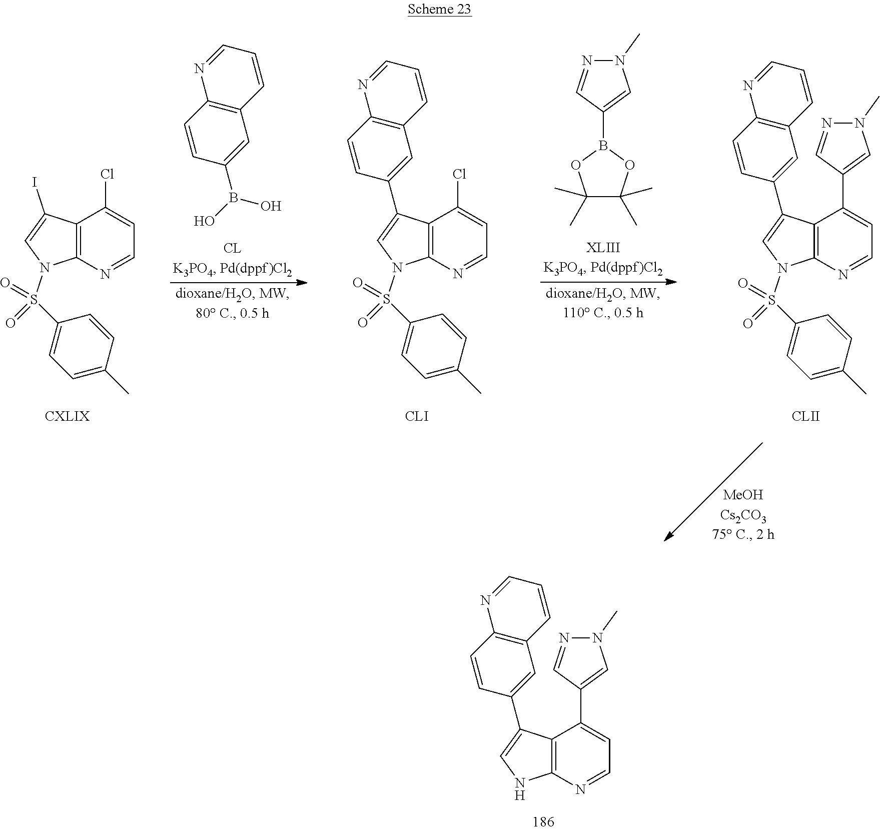

### Step 1

A mixture of N-tosyl-4-chloro-3-iodo-7-azaindole (CXLIX) (commercially available from Combi-Blocks Inc.) (500 mg, 1.16 mmol), quinoline-6-boronic acid (CL) (commercially available from Combi-Blocks Inc.) (220 mg, 1.27 mmol), a 2 M aqueous solution of K₃PO₄ (1.7mL, 3.4 mmol) and Pd(dppf)Cl₂ (50 mg, 0.06 mmol) in 1,4-dioxane (10 mL) was purged with N₂ gas for 5 min. The mixture was stirred and irradiated with microwave at 80° C. for 30 min. The reaction mixture was cooled, and organic layer was separated, concentrated. The crude product was purified by silica gel chromatography (0→10% MeOH/CHCl₃) to afford 6-[4-chloro-1-(4-methylphenyl)sulfonylpyrrolo[2,3-b]pyridin-3-yl]quinoline (CLI) (400 mg, 0.922 mmol, 79.8% yield) as a beige solid. ESIMS found for C₂₃H₁₆ClN₃O₂S m/z 434.0 (M+H).

### Step 2

A mixture of 6-[4-chloro-1-(4-methylphenyl)sulfonylpyrrolo[2,3-b]pyridin-3-yl]quinoline (CLI) (50 mg, 0.12 mmol), 1-methyl-4-(4,4,5,5-tetramethyl-1,3,2-dioxaborolan-2-yl)pyrazole (XLIII) (25 mg, 0.12 mmol), a 2 M aqueous solution of K₃PO₄ (0.17 mL, 0.34 mmol) and Pd(dppf)Cl₂ (5 mg, 0.01 mmol) in 1,4-dioxane (2 mL) was purged with N₂ gas for 5 min. The mixture was stirred and irradiated with microwave at 110° C. for 30 min. The reaction mixture was cooled, and organic layer was separated, concentrated to afford crude 6-[1-(4-methylphenyl)sulfonyl-4-(1-methylpyrazol-4-yl)pyrrolo[2,3-b]pyridin-3-yl]quinoline (CLII) (60 mg, 0.1251 mmol, 108.6% yield) as a dark brown solid which was used in the next step without further purification. ESIMS found for C₂₇H₂₁N₅O₂S m/z 480.1 (M+H).

### Step 3

A suspension of Cs₂CO₃ (205 mg, 0.63 mmol) and 6-[1-(4-methylphenyl)sulfonyl-4-(1-methylpyrazol-4-yl)pyrrolo[2,3-b]pyridin-3-yl]quinoline (CLII) (60 mg, 0.13 mmol) in MeOH (0.94 mL) was heated at 75° C. for 2 h. The reaction mixture was cooled and concentrated under vacuum. The crude was then purified by flash column chromatography (0→5% MeOH/CHCl₃). The desired fractions were concentrated to dryness in vacuo and the residue triturated in ether. The resulting solid was filtered and dried under high vacuo to afford 6-(4-(1-methyl-1H-pyrazol-4-yl)-1H-pyrrolo[2,3-b]pyridin-3-yl) quinoline (186) (20 mg, 0.062 mmol, 49.1% yield) as a beige solid. ¹H NMR (499 MHz, DMSO-d₆) δ ppm 3.39 (3H, s), 7.10 (1H, d, J=4.94 Hz), 7.15 (1H, s), 7.20 (1H, s), 7.45 (1H, dd, J=8.64, 2.06 Hz), 7.50 (1H, dd, J=8.23, 4.12 Hz), 7.57 (1H, d, J=1.92 Hz), 7.76 (1H, d, J=2.47 Hz), 7.79 (1H, d, J=8.78 Hz), 8.08-8.16 (1H, m), 8.28 (1H, d, J=4.67 Hz), 8.85 (1H, dd, J=4.25, 1.78 Hz), 12.07 (1H, br s); ESIMS found for C₂₀H₁₅N₅ m/z 326.1 (M+1).

### Example 3

Preparation of 6-(5-methyl-1H-pyrrolo[2,3-b]pyridin-3-yl)imidazo[1,2-a]pyridine (280) is depicted below in Scheme 24.

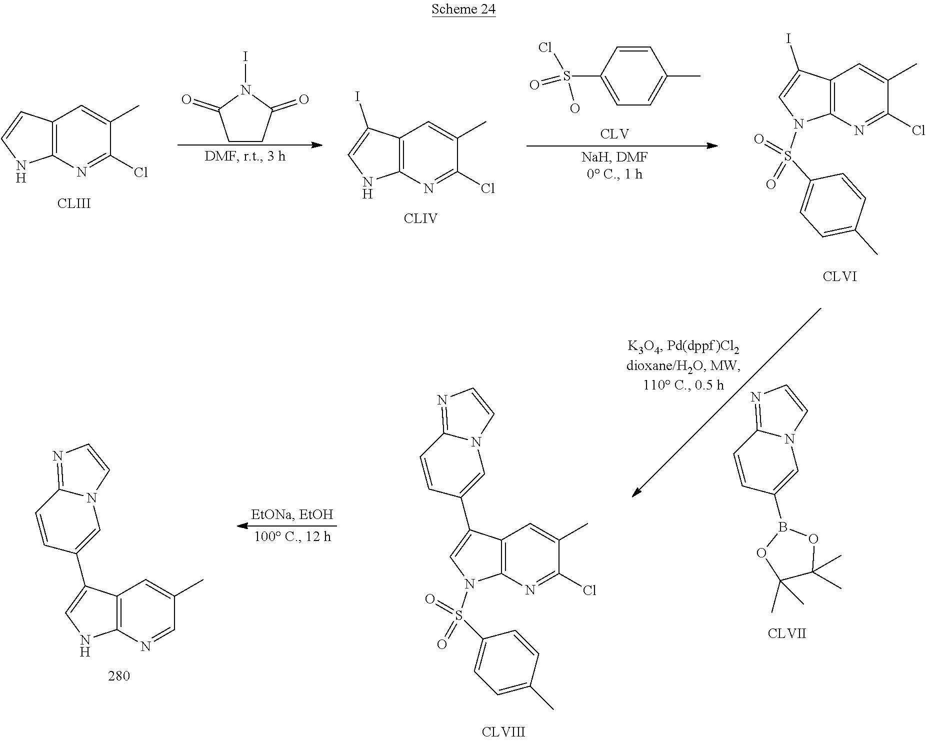

### Step 1

A mixture of 6-chloro-5-methyl-1H-pyrrolo[2,3-b]pyridine (CLIII) (commercially available from PharmaBlock (USA), Inc.) (1.05 g, 6.3 mmol), and 1-iodopyrrolidine-2,5-dione (1.5 g, 6.67 mmol) in DMF (5 mL) was stirred at room temperature for 3 h. Reaction mixture was concentrated to remove most of the DMF and the residue was triturated with water, resulting solids were filtered, washed with MeOH, and dried to obtain 6-chloro-3-iodo-5-methyl-1H-pyrrolo[2,3-b]pyridine (CLIV) (1.57 g, 5.368 mmol, 85.2% yield) as a beige solid. ESIMS found for C₈H₆ClIN₂ m/z 292.9 (M+H).

### Step 2

To a mixture of 6-chloro-3-iodo-5-methyl-1H-pyrrolo[2,3-b]pyridine (CLIV) (1.55 g, 5.3 mmol) in dry DMF (15 mL) was added NaH (500 mg, 12.5 mmol) portionwise under N₂ at 0° C. The mixture was stirred at 0° C. for 30 min. Tosyl chloride (CLV) (2.2 g, 11.54 mmol) was added portionwise and stirring was continued at 0° C. for 1 h. Reaction mixture was slowly diluted with water, precipitated solids were filtered, washed with aqueous saturated NaHC₃ and water. The collected solids were dried under high vacuum to obtain 6-chloro-3-iodo-5-methyl-1-(4-methylphenyl)sulfonylpyrrolo[2,3-b]pyridine (CLVI) (2.1 g, 4.701 mmol, 88.7% yield) as a beige solid. ESIMS found for C₁₅H₁₂ClIN₂O₂S m/z 446.8 (M+H).

### Step 3

A mixture of 6-chloro-3-iodo-5-methyl-1-(4-methylphenyl)sulfonylpyrrolo [2,3-b]pyridine (CLVI) (500 mg, 1.12 mmol), imidazo[1,2-a]pyridin-6-ylboronic acid, pinacol ester (CLVII) (commercially available from J&W Pharmlab) (300 mg, 1.23 mmol), a 2 M aqueous solution of K₃PO₄ (1.7 mL, 3.4 mmol) and Pd(dppf)Cl₂ (45 mg, 0.06 mmol) in 1,4-dioxane (20 mL) was purged with N₂ gas for 5 min. The mixture was stirred and irradiated with microwave at 110° C. for 30 min. The reaction mixture was cooled, and organic layer was separated and concentrated. The crude product was purified by silica gel chromatography (0→10% MeOH/CHCl₃) to afford 6-chloro-3-imidazo[1,2-a]pyridin-6-yl-5-methyl-1-(4-methylphenyl)sulfonylpyrrolo[2,3-b]pyridine (CLVIII) (410 mg, 0.938 mmol, 83.8% yield) as a beige solid. ESIMS found for C₂₂H₁₇ClN₄O₂S m/z 437.0 (M+1).

### Step 4

A solution of 6-chloro-3-imidazo[1,2-a]pyridin-6-yl-5-methyl-1-(4-methylphenyl)sulfonylpyrrolo[2,3-b]pyridine (CLVIII) (100 mg, 0.23 mmol) in a solution of sodium ethoxide in EtOH (2 mL, 5.1 mmol) was heated in a sealed tube at 100° C. for 12 h. The reaction mixture was cooled to room temperature, carefully acidified with 6 N HCl, extracted with EtOAc, washed with brine and concentrated. The residue was purified by ISCO (0→10% 7 N NH₃ in MeOH/CHCl₃). Pure fractions were combined, concentrated, and the resulting solids was triturated with DCM/hexanes, filtered and dried to produce 6-(5-methyl-1H-pyrrolo[2,3-b]pyridin-3-yl)imidazo[1,2-a]pyridine (280) (10 mg, 0.040 mmol, 17.6% yield) as a beige solid. ¹H NMR (499 MHz, DMSO-d₆) δ ppm 2.45 (3H, s), 7.56 (1H, d, J=1.09 Hz), 7.59-7.66 (2H, m), 7.91 (1H, d, J=2.46 Hz), 8.00 (1H, s), 8.15 (1H, d, J=1.92 Hz), 8.21 (1H, s), 8.90-8.96 (1H, m), 11.83 (1H, br s); ESIMS found for C₁₅H₁₂N₄ m/z 249.05 (M+1).

### Example 4

Preparation of 4,4-dimethyl-6-(5-((4-methylpiperazin-1-yl)methyl)-1H-pyrrolo[2,3-b] pyridin-3-yl)-3,4-dihydroisoquinolin-1(2H)-one (435) is depicted below in Scheme 25.

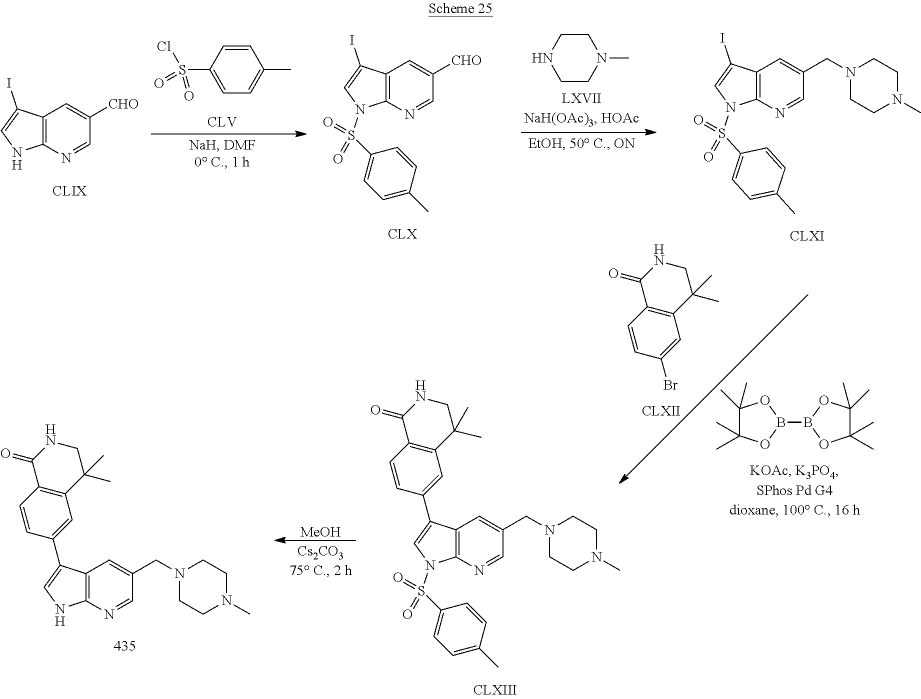

### Step 1

To a stirred solution of 3-iodo-1H-pyrrolo[2,3-b]pyridine-5-carbaldehyde (CLIX) (commercially available from Combi-Blocks Inc.) (1 g, 3.68 mmol) in DMF (12 mL) was added sodium hydride (180 mg, 4.41 mmol) at 0° C. and the mixture was stirred for 30 min. Then was added tosyl chloride (CLV) (770 mg, 4.05 mmol) in DMF (3 mL) and the mixture was stirred at room temperature for 3 h. The reaction mixture was added to water (100 mL) and stirred for 30 min. The resulting solids were collected by filtration and dried under high vacuo to obtain 3-iodo-1-(4-methylphenyl)sulfonylpyrrolo[2,3-b]pyridine-5-carbaldehyde (CLX) (1.93 g, 4.528 mmol, 123.2% yield) as an off-white solid. ESIMS found for C₁₅H₁₁IN₂O₃S m/z 426.85 (M+H).

### Step 2

A mixture of 1-methylpiperazine (LXVII) (0.26 mL, 2.34 mmol), HOAc (340 mg, 5.63 mmol) and 3-iodo-1-(4-methylphenyl)sulfonylpyrrolo[2,3-b]pyridine-5-carbaldehyde (CLX) (600 mg, 1.41 mmol) in a vial was stirred at room temperature for 20 min. NaH(OAc)₃ (450 mg, 2.12 mmol) was then added and the mixture was stirred at room temperature overnight. The reaction mixture was concentrated. EtOH (2 mL) and an additional 1-methylpiperazine (0.26 mL, 2.34 mmol) and NaH(OAc)₃ (450 mg, 2.12 mmol) was added to a vial and heated at 50° C. overnight. The reaction was evaporated under vacuum and the residue was absorbed on silica gel and was purified by ISCO (10→60% 10% 7 N NH₃ MeOH/CHCl₃) to obtain 3-iodo-1-(4-methylphenyl)sulfonyl-5-[(4-methylpiperazin-1-yl)methyl]pyrrolo[2,3-b]pyridine (CLXI) (250 mg, 0.490 mmol, 34.8% yield) as a light-yellow solid. ESIMS found for C₂₀H₂₃IN₄O₂S m/z 510.95 (M+H).

### Step 3

To a solution of bis(pinacolato)diboron (50 mg, 0.21 mmol) in 1,4-dioxane (1 mL) was added 6-bromo-4,4-dimethyl-3,4-dihydroisoquinolin-1(2H)-one (CLXII) (commercially available from Accela ChemBio Inc.) (50 mg, 0.19 mmol), KOAc (50 mg, 0.55 mmol) and SPhos Pd G4 (10 mg, 0.01 mmol). The mixture was purged with N₂ for 1 min before heating at 90° C. under N₂ for 2 h. The reaction was cooled to room temperature and a 2 M aqueous solution of K₃PO₄ (0.2 mL, 0.41 mmol), 3-iodo-1-(4-methylphenyl)sulfonyl-5-[(4-methylpiperazin-1-yl)methyl]pyrrolo[2,3-b]pyridine (CLXI) (90 mg, 0.18 mmol), and SPhos Pd G4 (10 mg, 0.01 mmol) were added. The reaction was purged with N₂ and then heated at 100° C. under N₂ for 16 h. The reaction was concentrated under vacuum and the residue purified via column chromatography (0→10% 7 N NH₃ in MeOH/CHCl₃) (4 g of silica gel) to yield 4,4-dimethyl-6-[1-(4-methylphenyl)sulfonyl-5-[(4-methylpiperazin-1-yl)methyl]pyrrolo[2,3-b]pyridin-3-yl]-2,3-dihydroisoquinolin-1-one (CLXIII) (52 mg, 0.093 mmol, 52.9% yield) as an amber viscous solid. ESIMS found for C₃₁H₃₅N₅O₃S m/z 558.1 (M+1).

### Step 4

A suspension of Cs₂CO₃ (150 mg, 0.45 mmol) and 4,4-dimethyl-6-[1-(4-methylphenyl)sulfonyl-5-[(4-methylpiperazin-1-yl)methyl]pyrrolo[2,3-b]pyridin-3-yl]-2,3-dihydroisoquinolin-1-one (CLXIII) (50 mg, 0.09 mmol) in MeOH (1 mL) was heated at 75° C. for 2 h. The reaction mixture was cooled to room temperature and concentrated. The crude was then purified by flash column chromatography (0→10% 7 N NH₃ in MeOH/CHCl₃). The desired fractions were concentrated to dryness in vacuo and the residue triturated in ether. The resulting solid was filtered and dried under high vacuo to produce 4,4-dimethyl-6-(5-((4-methylpiperazin-1-yl)methyl)-1H-pyrrolo[2,3-b]pyridin-3-yl)-3,4-dihydroisoquinolin-1(2H)-one (435) (5 mg, 0.012 mmol, 13.8% yield) as a tan solid. ¹H NMR (499 MHz, DMSO-d₆) δ ppm 1.36 (6H, s), 2.13 (3H, d, J=1.92 Hz), 2.24-2.33 (4H, m), 2.34-2.44 (4H, m), 3.21 (2H, d, J=2.74 Hz), 3.62 (2H, s), 6.40 (1H, dd, J=3.29, 1.92 Hz), 7.40-7.46 (1H, m), 7.66-7.70 (2H, m), 8.01 (1H, d, J=2.47 Hz), 8.18 (1H, s), 8.20 (1H, d, J=1.92 Hz), 12.00 (1H, br d, J=1.65 Hz); ESIMS found for C₂₄H₂₉N₅O m/z 404.1 (M+1).

### Example 5

Preparation of N-(5-fluoropyridin-3-yl)-3-(3-oxo-2,3-dihydrospiro[indene-1,4′-piperidin]-6-yl)-1H-pyrrolo[2,3-b]pyridine-5-carboxamide (764) is depicted below in Scheme 26.

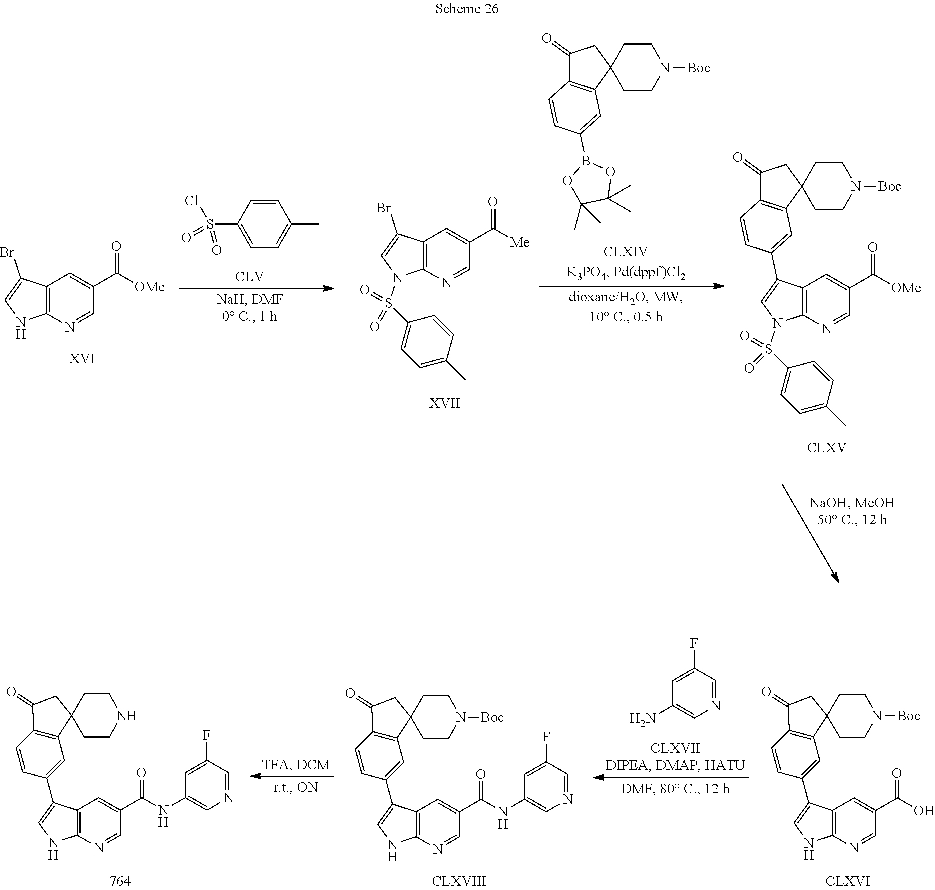

### Step 1

Starting with methyl 3-bromo-1H-pyrrolo[2,3-b]pyridine-5-carboxylate (XVI) (commercially available from PharmaBlock (USA), Inc.) protection was performed using procedure shown in Example 4, Scheme 25, Step 1 to yield methyl 3-bromo-1-(4-methylphenyl)sulfonylpyrrolo[2,3-b]pyridine-5-carboxylate (XVII) (1.45 g, 3.543 mmol, 86.1% yield) as a beige solid. ESIMS found for C₁₅H₁₂F₃N₇ m/z 348.1 (M+1). ESIMS found for C₁₆H₁₃BrN₂O₄S m/z 409.0 (M+H).

### Step 2

Methyl 3-bromo-1-(4-methylphenyl)sulfonylpyrrolo[2,3-b]pyridine-5-carboxylate (XVII) (290 mg, 0.71 mmol), tert-butyl 3-oxo-6-(4,4,5,5-tetramethyl-1,3,2-dioxaborolan-2-yl)spiro[2H-indene-1,4′-piperidine]-1′-carboxylate (CLXIV) (bromine analog is commercially available from 1 ClickChemistry Inc.) (250 mg, 0.59 mmol), Pd(dppf)Cl₂ (25 mg, 0.03 mmol) and a 2 M aqueous solution of K₃PO₄ (0.6 mL, 1.2 mmol) in 1,4-dioxane (5 mL) was purged with N₂ gas for 5 min. The mixture was stirred and irradiated with microwave at 110° C. for 30 min. The reaction mixture was cooled down to room temperature, organic layer was separated and concentrated, and resulting residue was purified by column chromatography (0→5% MeOH/CHCl₃) to obtain tert-butyl 6-[5-methoxycarbonyl-1-(4-methylphenyl)sulfonylpyrrolo[2,3-b]pyridin-3-yl]-3-oxospiro[2H-indene-1,4′-piperidine]-1′-carboxylate (CLXV) (350 mg, 0.556 mmol, 95.0% yield) as a beige solid. ESIMS found for C₃₄H₃₅N₃O₇S m/z 630.3 (M+H).

### Step 3

tert-Butyl 6-[5-methoxycarbonyl-1-(4-methylphenyl)sulfonylpyrrolo[2,3-b]pyridin-3-yl]-3-oxospiro[2H-indene-1,4′-piperidine]-1′-carboxylate (CLXV) (250 mg, 0.4 mmol) and a 2 M aqueous solution of NaOH (1 mL, 2. mmol) in MeOH (5 mL) was stirred at 50° C. for 12 h. Solvent was evaporated, resulting residue was diluted with water, neutralized with 2 N HCl, extracted with EtOAc, combined extracts were dried with anhydrous Na₂SO₄, and evaporated, to obtain 3-[1′-[(2-methylpropan-2-yl)oxycarbonyl]-1-oxospiro[2H-indene-3,4′-piperidine]-5-yl]-1H-pyrrolo[2,3-b]pyridine-5-carboxylic acid (CLXVI) (180 mg, 0.390 mmol, 98.2% yield) and as an off-white solids. ESIMS found for C₂₆H₂₇N₃O₅ m/z 462.2 (M+H).

### Step 4

To a suspension of 3-[1′-[(2-methylpropan-2-yl)oxycarbonyl]-1-oxospiro[2H-indene-3,4′-piperidine]-5-yl]-1H-pyrrolo[2,3-b]pyridine-5-carboxylic acid (CLXVI) (180 mg, 0.39 mmol), 3-amino-5-fluoropyridine (CLXVII) (40 mg, 0.39 mmol), HATU (220 mg, 0.58 mmol) and DMAP (0.g, 0.02 mmol) in DMF (1 mL) was added DIPEA (0.14 mL, 0.8 mmol). The resulting mixture was stirred at 80° C. for 12 h. The reaction mixture was cooled and diluted with water, precipitated solids were filtered and washed with water. The collected solids was dried under high vacuum to afford tert-butyl 6-[5-[(5-fluoropyridin-3-yl)carbamoyl]-1H-pyrrolo[2,3-b]pyridin-3-yl]-3-oxospiro[2H-indene-1,4′-piperidine]-1′-carboxylate (CLXVIII) (75 mg, 0.135 mmol, 34.6% yield) as a brown solid. ESIMS found for C₃₁H₃₀FN₅O₄ m/z 556.25 (M+1).

### Step 5

To a solution of TFA (0.5 mL, 6.49 mmol) in DCM (1 mL) was added tert-butyl 6-[5-[(5-fluoropyridin-3-yl)carbamoyl]-1H-pyrrolo[2,3-b]pyridin-3-yl]-3-oxospiro[2H-indene-1,4′-piperidine]-1′-carboxylate (CLXVIII) (75 mg, 0.13 mmol). The mixture was stirred at room temperature overnight. The solvent was evaporated and resulting residue was purified by column chromatography (0→10% 7 N NH₃ in MeOH/CHCl₃) to produce N-(5-fluoropyridin-3-yl)-3-(3-oxo-2,3-dihydrospiro[indene-1,4′-piperidin]-6-yl)-1H-pyrrolo[2,3-b]pyridine-5-carboxamide (764) (20 mg, 0.044 mmol, 32.5% yield) as a beige solid. ¹H NMR (499 MHz, DMSO-d₆) δ ppm 1.44 (2H, br d, J=12.62 Hz), 2.05 (2H, td, J=12.49, 3.84 Hz), 2.62-2.71 (2H, m), 2.64 (2H, s), 2.98 (2H, br d, J=11.53 Hz), 7.71 (1H, d, J=7.96 Hz), 7.89 (1H, dd, J=7.96, 1.37 Hz), 8.02 (1H, s), 8.25 (1H, dt, J=11.39, 2.26 Hz), 8.29 (1H, s), 8.34 (1H, d, J=2.74 Hz), 8.79-8.84 (1H, m), 8.88 (1H, d, J=1.92 Hz), 8.93 (1H, d, J=1.92 Hz), 10.85 (1H, br s); ESIMS found for C₂₆H₂₂FN₅O₂ m/z 456.2 (M+1).

### Example 6

Preparation of 3-(3-(1-methyl-1H-pyrazol-4-yl)pyrazolo[1,5-a]pyridin-5-yl)-5-(2-(4-methylpiperazin-1-yl)pyridin-4-yl)-1H-pyrrolo[2,3-b]pyridine (1384) is depicted below in Scheme 27.

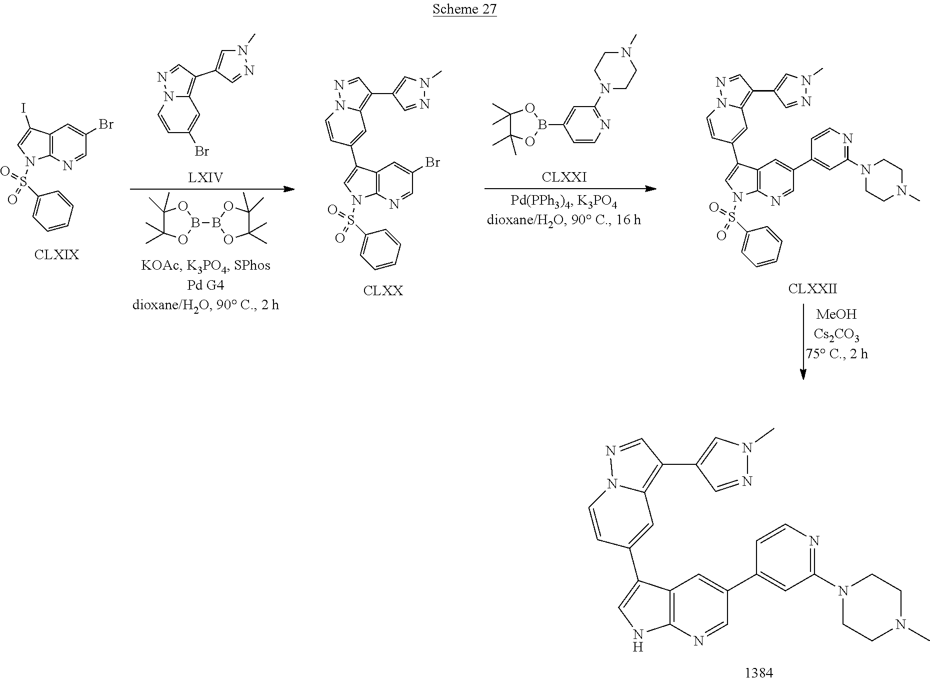

### Step 1

To a solution of 5-bromo-3-(1-methyl-1H-pyrazol-4-yl)pyrazolo[1,5-a]pyridine (LXIV) (70 mg, 0.250 mmol) in 1,4-dioxane (1 mL) was added KOAc (80 mg, 0.770 mmol) and a 2 M aqueous solution of K₃PO₄ (0.3 mL, 0.600 mmol). The mixture was bubbled with N₂ for 1 min before heating at 90° C. under N₂ for 2 h. The reaction was cooled to room temperature and 5-bromo-3-iodo-1-(phenylsulfonyl)-1H-pyrrolo[2,3-b]pyridine (CLXIX) (commercially available from CombiBlocks) (0.12 g, 0.250 mmol), bis(pinacolato)diboron (80 mg, 0.300 mmol), and SPhos Pd G4 (10 mg, 0.020 mmol) were added. The reaction was purged with N₂ and then heated at 100° C. under N₂ for 16 h. The reaction was concentrated under vacuum. The residue was purified via column chromatography (0→8% 7 N NH₃ in MeOH/CHCl₃) (12 g of silica gel) to yield 5-bromo-3-(3-(1-methyl-1H-pyrazol-4-yl)pyrazolo[1,5-a]pyridin-5-yl)-1-(phenylsulfonyl)-1H-pyrrolo[2,3-b]pyridine (CLXX) (105 mg, 0.197 mmol, 79.3% yield) as an amber viscous solid. ESIMS found for C₂₄H₁₇BrN₆O₂S m/z 532.9 (⁷⁹BrM+H).

### Step 2

To a stirred solution of 5-bromo-3-(3-(1-methyl-1H-pyrazol-4-yl)pyrazolo[1,5-a]pyridin-5-yl)-1-(phenylsulfonyl)-1H-pyrrolo[2,3-b]pyridine (CLXX) (110 mg, 0.200 mmol) in 1,4-dioxane (3 mL) was added a 2 M aqueous solution of K₃PO₄ (0.25 mL, 0.500 mmol). Reaction mixture was purged with argon before adding Pd(PPh₃)₄ (10 mg, 0.010 mmol) and 1-methyl-4-(4-(4,4,5,5-tetramethyl-1,3,2-dioxaborolan-2-yl)pyridin-2-yl)piperazine (CLXXI) (commercially available from CombiBlocks) (59.7 mg, 0.197 mmol). The reaction heated at 90° C. for 16 h under argon. The reaction was concentrated under vacuum and the residue purified via column chromatography (0→8% 7 N NH₃ in MeOH/CHCl₃) (12 g of silica gel) to produce 3-(3-(1-methyl-1H-pyrazol-4-yl)pyrazolo[1,5-a]pyridin-5-yl)-5-(2-(4-methylpiperazin-1-yl)pyridin-4-yl)-1-(phenylsulfonyl)-1H-pyrrolo[2,3-b]pyridine (CLXXII) (88 mg, 0.140 mmol, 71.0% yield) as a light orange solid. ESIMS found for C₃₄H₃₁N₉O₂S m/z 630.1 (M+H).

### Step 3

A suspension of Cs₂CO₃ (230 mg, 0.700 mmol) and 3-(3-(1-methyl-1H-pyrazol-4-yl)pyrazolo[1,5-a]pyridin-5-yl)-5-(2-(4-methylpiperazin-1-yl)pyridin-4-yl)-1-(phenylsulfonyl)-1H-pyrrolo[2,3-b]pyridine (CLXXII) (90 mg, 0.140 mmol) in MeOH (1 mL) was heated at 75° C. for 2 h. The reaction mixture was cooled to room temperature and concentrated under vacuum. The crude was then purified by flash column chromatography (0→10% 7 N NH₃ in MeOH/CHCl₃). The desired fractions were concentrated to dryness in vacuo and the residue triturated in ether. The resulting solid was filtered and dried under high vacuo to afford 3-(3-(1-methyl-1H-pyrazol-4-yl)pyrazolo[1,5-a]pyridin-5-yl)-5-(2-(4-methylpiperazin-1-yl)pyridin-4-yl)-1H-pyrrolo[2,3-b]pyridine (1384) (5 mg, 0.010 mmol, 7.3% yield) as a light-yellow solid. ¹H NMR (499 MHz, DMSO-d₆) δ ppm 2.23 (3H, s), 2.42 (4H, t, J=4.94 Hz), 3.56-3.62 (4H, m), 3.91 (3H, s), 7.08-7.14 (1H, m), 7.22 (1H, s), 7.39 (1H, dd, J=7.27, 1.78 Hz), 7.94 (1H, s), 8.09 (1H, d, J=0.82 Hz), 8.20 (1H, d, J=5.21 Hz), 8.20 (1H, s), 8.22 (1H, s), 8.26 (1H, s), 8.63 (1H, d, J=1.92 Hz), 8.66-8.71 (2H, m), 12.30 (1H, s); ESIMS found for C₂₈H₂₇N₉ m/z 490.1 (M+1).

### Example 7

Preparation of 6-(6-cyclopropyl-1H-pyrrolo[2,3-b]pyridin-3-yl)-4-fluoro-2-methyl-1-(1-methylpiperidin-4-yl)-1H-benzo[d]imidazole (1502) is depicted below in Scheme 28.

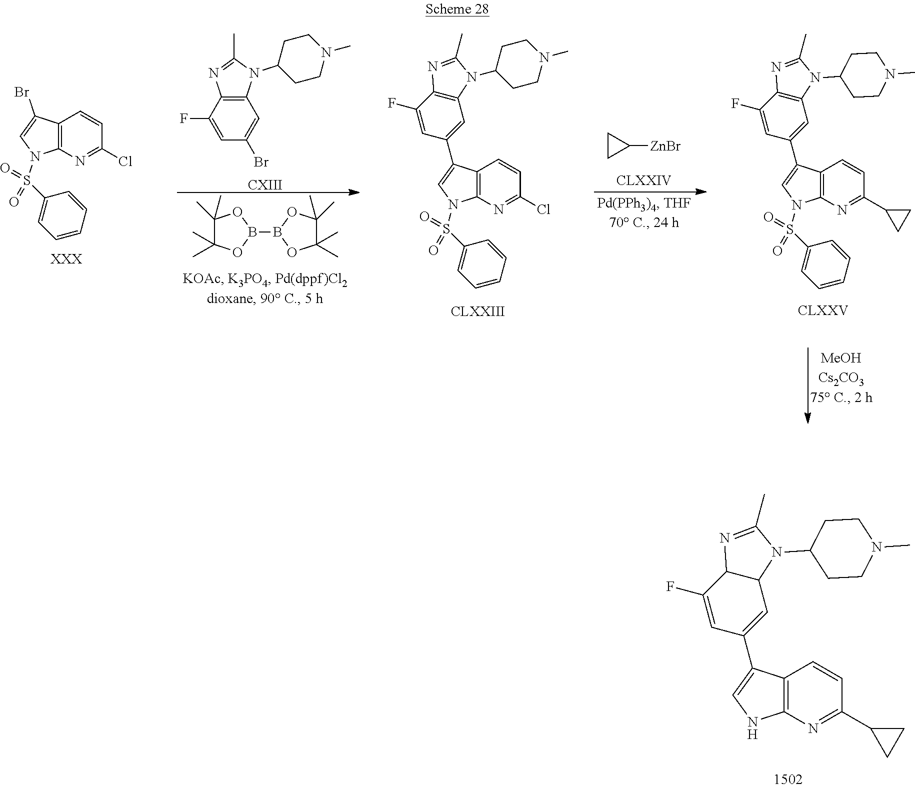

### Step 1a-b

A mixture of 6-bromo-4-fluoro-2-methyl-1-(1-methylpiperidin-4-yl)-1H-benzo[d]imidazole (XXX) (150 mg, 0.460 mmol), bis(pinacolato)diboron (175 mg, 0.690 mmol), Pd(dppf)Cl₂ (37.6 mg, 0.050 mmol) and KOAc (135.4 mg, 1.38 mmol) was taken in 1,4-dioxane (2 mL). N₂ gas was bubbled into the mixture for 3 min and then was heated to 90° C. for 5 h. LC/MS of the mixture showed completion of the reaction. ESIMS found for C₂₀H₂₉BFN₃O₂ m/z 374.2 (M+H).

To the reaction mixture was added 3-bromo-6-chloro-1-(phenylsulfonyl)-1H-pyrrolo[2,3-b]pyridine (CXIII) (commercially available from Advanced ChemBlocks Inc.) (171 mg, 0.460 mmol), Pd(dppf)Cl₂ (37.6 mg, 0.050 mmol) and a 2 M aqueous solution of K₃PO₄ (0.46 mL, 0.920 mmol). N₂ gas was bubbled for 5 min and then was heated to 90° C. for 16 h. The reaction mixture was added to a solution of saturated aqueous NaHCO₃ (40 mL), extracted with EtOAc, organics separated, washed with water and brine. The organics were separated, dried over anhydrous Na₂SO₄, solvents concentrated, and the crude was purified by ISCO (0→30% MeOH/CHCl₃) to obtain 6-(6-chloro-1-(phenylsulfonyl)-1H-pyrrolo[2,3-b]pyridin-3-yl)-4-fluoro-2-methyl-1-(1-methylpiperidin-4-yl)-1H-benzo[d]imidazole (CLXXIII) (109 mg, 0.203 mmol, 44.1% yield) as a beige solid. ESIMS found for C₂₇H₂₅ClFN₅O₂S m/z 538.2 (M+H).

### Step 2

To a stirred solution of Pd(PPh₃)₄ (20 mg, 0.020 mmol) and 6-(6-chloro-1-(phenylsulfonyl)-1H-pyrrolo[2,3-b]pyridin-3-yl)-4-fluoro-2-methyl-1-(1-methylpiperidin-4-yl)-1H-benzo[d]imidazole (CLXXIII) (110 mg, 0.200 mmol) in dry THF (0.5 mL) was added under N₂ cyclopropylzinc(II) bromide (CLXXIV) (0.61 mL, 0.310 mmol) and the mixture was heated to 70° C. for 16 h. LC/MS showed only 20% of the conversion. Additional equivalent of bromo(cyclopropyl)zinc (0.61 mL, 0.310 mmol) was added and the mixture was heated to 70° C. for 24 h. The reaction mixture was cooled, added to a solution of saturated aqueous NaHCO₃ and extracted with EtOAc. The organic layer was separated, washed with water, brine and dried over anhydrous Na₂SO₄, solvents removed, and the crude was purified by ISCO (0→30% MeOH/CHCl₃) followed by prep TLC to obtain 6-(6-cyclopropyl-1-(phenylsulfonyl)-1H-pyrrolo[2,3-b]pyridin-3-yl)-4-fluoro-2-methyl-1-(1-methylpiperidin-4-yl)-1H-benzo[d]imidazole (CLXXV) (63 mg, 0.116 mmol, 57.2% yield) as a beige solid. ESIMS found for C₃₀H₃₀FN₅O₂S m/z 544. (M+H).

### Step 3

A suspension of Cs₂CO₃ (189 mg, 0.580 mmol) and 6-(6-cyclopropyl-1-(phenylsulfonyl)-1H-pyrrolo[2,3-b]pyridin-3-yl)-4-fluoro-2-methyl-1-(1-methylpiperidin-4-yl)-1H-benzo[d]imidazole (CLXXV) (63 mg, 0.120 mmol) in MeOH (1 mL) was heated at 75° C. for 5 h. The reaction mixture was cooled to room temperature and concentrated under vacuum. The residue was dissolved in a mixture of CHCl₃/MeOH and then purified by preparative TLC (6% 7 N NH₃ in MeOH/CHCl₃) to afford 6-(6-Cyclopropyl-1H-pyrrolo[2,3-b]pyridin-3-yl)-4-fluoro-2-methyl-1-(1-methylpiperidin-4-yl)-1H-benzo[d]imidazole (1502) (9 mg, 0.022 mmol, 19.2% yield) as a beige solid. ¹H NMR (499 MHz, DMSO-d₆) δ ppm 0.94-0.99 (4H, m), 1.82-1.90 (2H, m), 2.10-2.20 (3H, m), 2.25 (3H, s), 2.42 (2H, qd, J=12.26, 3.57 Hz), 2.61 (3H, s), 2.94 (2H, br d, J=11.25 Hz), 4.32 (1H, tt, J=12.35, 4.39 Hz), 7.12 (1H, d, J=8.23 Hz), 7.22-7.28 (1H, m), 7.58 (1H, d, J=1.10 Hz), 7.77 (1H, d, J=2.47 Hz), 8.07 (1H, d, J=7.96 Hz), 11.73 (1H, br s); ESIMS found for C₂₄H₂₆FN₅ m/z 404.2 (M+1).

### Example 8

Preparation of N-(3-(3-oxo-2,3-dihydrospiro[indene-1,4′-piperidin]-6-yl)-1H-pyrrolo [2,3-b]pyridin-6-yl)isonicotinamide (1898) and N-(3-(1′-methyl-3-oxo-2,3-dihydrospiro[indene-1,4′-piperidin]-6-yl)-1H-pyrrolo[2,3-b]pyridin-6-yl)isonicotinamide (1899) are depicted below in Scheme 29.

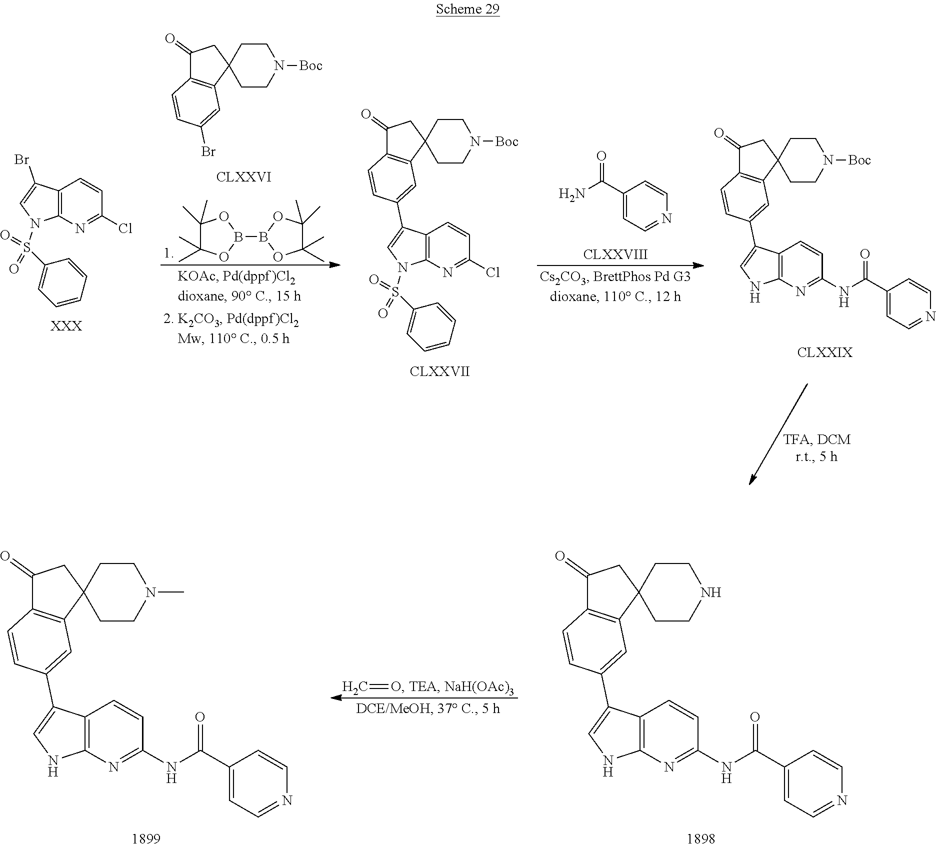

### Steps 1-2

A mixture of KOAc (240 mg, 2.45 mmol), tert-butyl 6-bromo-3-oxo-2,3-dihydrospiro[indene-1,4-piperidine]-1-carboxylate (CLXXVI) (commercially available from 1 ClickChemistry Inc.) (300 mg, 0.79 mmol), bis(pinacolato)diboron (240 mg, 0.95 mmol) and Pd(dppf)Cl₂ (30 mg, 0.04 mmol) in 1,4-dioxane (10 mL) was bubbled with N₂ gas for 5 min. The reaction was then heated to 90° C. for 5 h. Reaction mixture was cooled before adding 1-(benzenesulfonyl)-3-bromo-6-chloro-1H-pyrrolo[2,3-b]pyridine (XXX) (commercially available from Advanced ChemBlocks Inc.) (350 mg, 0.94 mmol), a 2 M aqueous solution of K₂CO₃ (1.2 mL, 2.4 mmol), and another portion of Pd(dppf)Cl₂ (30 mg, 0.04 mmol). N₂ gas was bubbled into the mixture for 5 min. and the mixture was stirred and irradiated with microwave at 110° C. for 30 min. The organic layer was separated and concentrated, purified by ISCO (0-100% EtOAc/hexane) to obtain tert-butyl 6-[1-(benzenesulfonyl)-6-chloropyrrolo[2,3-b]pyridin-3-yl]-3-oxospiro[2H-indene-1,4′-piperidine]-1′-carboxylate (CLXXVII) (317 mg, 0.535 mmol, 67.9% yield) as a beige solid. ESIMS found for C₃₁H₃₀ClN₃O₅S m/z 592.05 (M+H).

### Step 3

A mixture of tert-butyl 6-[1-(benzenesulfonyl)-6-chloropyrrolo[2,3-b]pyridin-3-yl]-3-oxospiro[2H-indene-1,4′-piperidine]-1′-carboxylate (CLXXVII) (317 mg, 0.54 mmol), isonicotinamide (CLXXVIII) (commercially available from Ark Pharma Scientific Limited) (80 mg, 0.66 mmol), BrettPhos Pd G3 (50 mg, 0.06 mmol), and Cs₂CO₃ (360 mg, 1.1 mmol) in 1,4-dioxane (5 mL) was purged with N₂ gas for 5 min. The mixture was stirred at 110° C. for12 h. The reaction mixture was cooled to room temperature, filtered, and concentrated. The crude product was purified by silica gel chromatography (0→15% MeOH/CHCl₃) to afford tert-butyl 3-oxo-6-[6-(pyridine-4-carbonylamino)-1H-pyrrolo[2,3-b]pyridin-3-yl]spiro[2H-indene-1,4′-piperidine]-1′-carboxylate (CLXXIX) (75 mg, 0.140 mmol, 26.1% yield) as a beige solid. ESIMS found for C₃₁H₃₁N₅O₄ m/z 538.1 (M+H).

### Step 4

To a solution of tert-butyl 3-oxo-6-[6-(pyridine-4-carbonylamino)-1H-pyrrolo[2,3-b]pyridin-3-yl]spiro[2H-indene-1,4′-piperidine]-1′-carboxylate (CLXXIX) (75 mg, 0.14 mmol) in DCM (5 mL) was added TFA (0.2 mL, 2.6 mmol). The reaction mixture was stirred at room temperature for 5 h. Reaction mixture was concentrated, and the residue was purified by column chromatography (0→10% 7 N NH₃ in MeOH/CHCl₃). The pure fractions were combined and concentrated, resulting solids were triturated with DCM, filtered, and dried to give N-(3-(3-oxo-2,3-dihydrospiro[indene-1,4′-piperidin]-6-yl)-1H-pyrrolo[2,3-b]pyridin-6-yl)isonicotinamide (1898) (42 mg, 0.096 mmol, 68.8% yield) as a beige solid. ¹H NMR (499 MHz, DMSO-d₆) δ ppm 1.46 (2H, br d, J=12.32 Hz), 2.07 (2H, td, J=12.73, 4.11 Hz), 2.64 (2H, s), 2.70 (2H, br t, J=11.77 Hz), 3.02 (2H, br d, J=11.77 Hz), 7.65 (1H, d, J=7.94 Hz), 7.85 (1H, dd, J=8.08, 1.23 Hz), 7.93-7.95 (2H, m), 7.96 (1H, s), 8.05 (1H, d, J=8.76 Hz), 8.11 (1H, s), 8.45 (1H, d, J=8.49 Hz), 8.75-8.80 (2H, m), 11.03 (1H, br s), 12.03 (1H, br s); ESIMS found for C₂₆H₂₃N₅O₂ m/z 438.1 (M+1).

### Step 5

A mixture of N-[3-(1-oxospiro[2H-indene-3,4′-piperidine]-5-yl)-1H-pyrrolo[2,3-b]pyridin-6-yl]pyridine-4-carboxamide (1898) (37 mg, 0.08 mmol), formaldehyde (30 μL, 0.38 mmol) and TEA (40 μL, 0.29 mmol) in DCE (3 mL) and MeOH (1 mL) was stirred for 30 min at room temperature. NaH(OAc)₃ (90 mg, 0.42 mmol) was then added and the mixture was stirred at 37° C. for 5 h. The reaction mixture was concentrated, and the resulting residue was adsorbed on silica gel and purified by column chromatography (0→10% MeOH/CHCl₃). Pure fractions were collected and further purified by Preparative TLC (20% MeOH/CHCl₃) to produce N-(3-(1′-methyl-3-oxo-2,3-dihydrospiro[indene-1,4′-piperidin]-6-yl)-1H-pyrrolo[2,3-b]pyridin-6-yl)isonicotinamide (1899) (18 mg, 0.040 mmol, 47.1% yield) as a beige solid. ¹H NMR (499 MHz, DMSO-d₆) δ ppm 1.50 (2H, br d, J=12.05 Hz), 2.01-2.09 (2H, m), 2.20 (2H, br dd, J=12.87, 3.56 Hz), 2.24 (3H, s), 2.58 (2H, s), 2.83 (2H, br d, J=11.50 Hz), 7.65 (1H, d, J=7.94 Hz), 7.86 (1H, dd, J=7.94, 1.37 Hz), 7.93-7.95 (2H, m), 8.00 (1H, s), 8.04 (1H, d, J=8.76 Hz), 8.13 (1H, d, J=2.46 Hz), 8.47 (1H, d, J=8.76 Hz), 8.76-8.79 (2H, m), 11.02 (1H, s), 12.02 (1H, br s); ESIMS found for C₂₇H₂₅N₅O₂ m/z 452.1 (M+1).

The following compounds were prepared in accordance with the procedures described in the above Schemes 1-29.

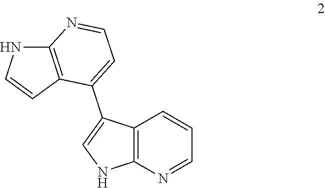 [SMILES: c1cnc2[nH]cc(-c3ccnc4[nH]ccc34)c2c1]

### 1H,1′H-3,4′-Bipyrrolo[2,3-b]pyridine 2

Beige solid (8 mg, 0.034 mmol, 16.7% yield). ¹H NMR (499 MHz, DMSO-d₆) δ ppm 6.67 (1H, dd, J=3.57, 1.92 Hz), 7.20 (1H, dd, J=7.82, 4.53 Hz), 7.33 (1H, d, J=4.94 Hz), 7.45-7.52 (1H, m), 8.04 (1H, s), 8.24-8.28 (2H, m), 8.33 (1H, dd, J=4.67, 1.37 Hz), 11.67 (1H, br s); ESIMS found for C₁₄H₁₀N₄ m/z 235.0 (M+1).

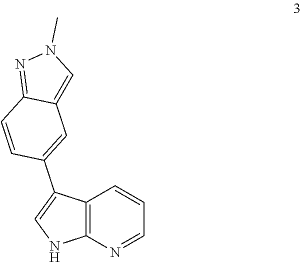 [SMILES: Cn1cc2cc(-c3c[nH]c4ncccc34)ccc2n1]

### 2-Methyl-5-(1H-pyrrolo[2,3-b]pyridin-3-yl)-2H-indazole 3

Dark yellow solid (35.0 mg, 0.141 mmol, 34.4% yield). ¹H NMR (499 MHz, DMSO-d₆) δ ppm 4.18 (3H, s), 7.16 (1H, dd, J=7.82, 4.53 Hz), 7.59-7.68 (2H, m), 7.84 (1H, d, J=2.47 Hz), 7.98 (1H, s), 8.27 (1H, dd, J=4.67, 1.37 Hz), 8.30 (1H, s), 8.33 (1H, dd, J=7.96, 1.10 Hz), 11.82 (1H, br s); ESIMS found for C₁₅H₁₂N₄ m/z 249.0 (M+1).

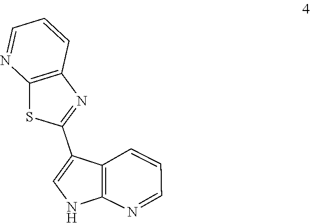 [SMILES: c1cnc2sc(-c3c[nH]c4ncccc34)nc2c1]

### 2-(1H-Pyrrolo[2,3-b]pyridin-3-yl)thiazolo[5,4-b]pyridine 4

Beige solid (20 mg, 0.079 mmol, 51.9% yield). ¹H NMR (499 MHz, DMSO-d₆) δ ppm 7.33 (1H, dd, J=7.96, 4.67 Hz), 7.55 (1H, dd, J=7.96, 4.67 Hz), 8.32 (1H, dd, J=8.10, 1.51 Hz), 8.39 (1H, dd, J=4.67, 1.65 Hz), 8.50 (1H, s), 8.52 (1H, dd, J=4.67, 1.37 Hz), 8.71 (1H, dd, J=7.96, 1.65 Hz), 12.61 (1H, br s); ESIMS found for C₁₃H₈N₄S m/z 253.0 (M+1).

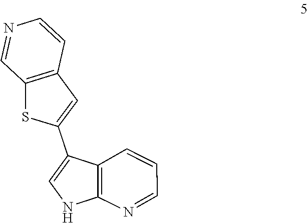 [SMILES: c1cnc2[nH]cc(-c3cc4ccncc4s3)c2c1]

### 2-(1H-Pyrrolo[2,3-b]pyridin-3-yl)thieno[2,3-c]pyridine 5

Yellow solid (30 mg, 0.119 mmol, 46.7% yield). ¹H NMR (499 MHz, DMSO-d₆) δ ppm 7.28 (1H, dd, J=8.10, 4.53 Hz), 7.74 (1H, dd, J=5.35, 0.96 Hz), 7.82 (1H, s), 8.18 (1H, s), 8.36 (1H, dd, J=4.67, 1.37 Hz), 8.43 (1H, d, J=5.21 Hz), 8.46 (1H, dd, J=7.96, 1.37 Hz), 9.15 (1H, s), 12.32 (1H, br s); ESIMS found for C₁₄H₉N₃S m/z 252.1 (M+1).

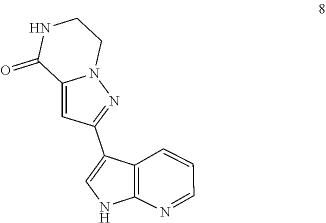 [SMILES: O=C1NCCn2nc(-c3c[nH]c4ncccc34)cc21]

### 2-(1H-Pyrrolo[2,3-b]pyridin-3-yl)-6,7-dihydropyrazolo[1,5-a]pyrazin-4(5H)-one 8

Off-white solid (6.1 mg, 0.024 mmol, 17.5% yield). ¹H NMR (499 MHz, DMSO-d₆) δ ppm 3.61-3.69 (2H, m), 4.32-4.38 (2H, m), 7.12 (1H, s), 7.14 (1H, dd, J=7.82, 4.53 Hz), 7.97 (1H, d, J=2.47 Hz), 8.19 (1H, br s), 8.26 (1H, dd, J=4.67, 1.37 Hz), 8.47 (1H, dd, J=7.96, 1.65 Hz), 11.82 (1H, br s); ESIMS found for C₁₃H₁₁N₅O m/z 254.1 (M+1).

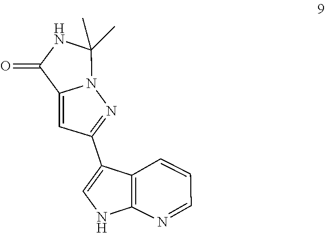 [SMILES: CC1(C)NC(=O)c2cc(-c3c[nH]c4ncccc34)nn21]

### 6,6-Dimethyl-2-(1H-pyrrolo[2,3-b]pyridin-3-yl)-5,6-dihydro-4H-imidazo[1,5-b]pyrazol-4-one 9

Beige solid (3 mg, 0.011 mmol, 13.1% yield). ¹H NMR (499 MHz, DMSO-d₆) δ ppm 1.70 (6H, s), 6.94 (1H, s), 7.16 (1H, dd, J=7.96, 4.67 Hz), 7.97 (1H, d, J=2.47 Hz), 8.27 (1H, dd, J=4.67, 1.65 Hz), 8.49 (1H, dd, J=7.96, 1.37 Hz), 9.43 (1H, s), 11.86 (1H, br s); ESIMS found for C₁₄H₁₃N₅O m/z 268.1 (M+1).

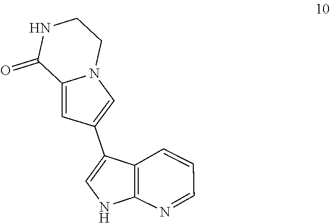 [SMILES: O=C1NCCn2cc(-c3c[nH]c4ncccc34)cc21]

### 7-(1H-Pyrrolo[2,3-b]pyridin-3-yl)-3,4-dihydropyrrolo[1,2-a]pyrazin-1(2H)-one 10

Beige solid (20 mg, 0.079 mmol, 19.4% yield). ¹H NMR (499 MHz, DMSO-d₆) δ ppm 3.53 (2H, br t, J=7.14 Hz), 4.10-4.15 (2H, m), 6.99 (1H, d, J=1.65 Hz), 7.10 (1H, dd, J=7.96, 4.67 Hz), 7.45 (1H, d, J=1.65 Hz), 7.65 (1H, br s), 7.68 (1H, d, J=2.47 Hz), 8.20 (1H, d, J=7.96 Hz), 8.22 (1H, dd, J=4.67, 1.37 Hz), 11.59 (1H, br s); ESIMS found for C₁₄H₁₂N₄O m/z 253.1 (M+1).

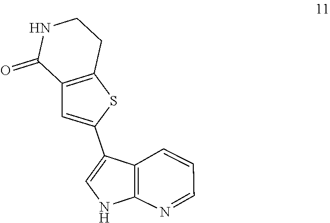 [SMILES: O=C1NCCc2sc(-c3c[nH]c4ncccc34)cc21]

### 2-(1H-Pyrrolo[2,3-b]pyridin-3-yl)-6,7-dihydrothieno[3,2-c]pyridin-4(5H)-one 11

Beige solid (20 mg, 0.074 mmol, 18.1% yield). ¹H NMR (499 MHz, DMSO-d₆) δ ppm 3.01 (2H, br t, J=6.59 Hz), 3.48 (2H, br t, J=5.35 Hz), 7.19 (1H, br dd, J=7.96, 4.67 Hz), 7.43 (1H, s), 7.58 (1H, br s), 7.90 (1H, br s), 8.21 (1H, br d, J=7.96 Hz), 8.29 (1H, br d, J=3.84 Hz), 12.00 (1H, br s); ESIMS found for C₁₄H₁₁N₃OS m/z 270.0 (M+1).

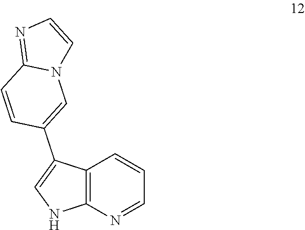 [SMILES: c1cnc2[nH]cc(-c3ccc4nccn4c3)c2c1]

### 6-(1H-Pyrrolo[2,3-b]pyridin-3-yl)imidazo[1,2-a]pyridine 12

Yellow solid (16 mg, 0.068 mmol, 30.1% yield). ¹HNMR (500 MHz, DMSO-d₆) δ ppm 7.20 (1H, dd, J=8.10, 4.53 Hz), 7.56 (1H, d, J=1.10 Hz), 7.60-7.69 (2H, m), 7.97 (1H, s), 7.99 (1H, s), 8.31 (1H, dd, J=4.67, 1.37 Hz), 8.41 (1H, dd, J=7.96, 1.37 Hz), 8.95 (1H, s), 11.98 (1H, br s); ESIMS found for C₁₄H₁₀N₄ m/z 235.1 (M+1).

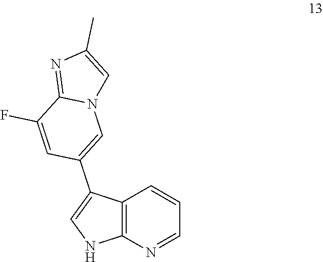 [SMILES: Cc1cn2cc(-c3c[nH]c4ncccc34)cc(F)c2n1]

### 8-Fluoro-2-methyl-6-(1H-pyrrolo[2,3-b]pyridin-3-yl)imidazo[1,2-a]pyridine 13

Grey solid (12.5 mg, 0.047 mmol, 10.8% yield). ¹H NMR (499 MHz, DMSO-d₆) δ ppm 2.37 (3H, s), 7.20 (1H, dd, J=7.96, 4.39 Hz), 7.54 (1H, dd, J=12.62, 1.37 Hz), 7.80-7.87 (1H, m), 8.01 (1H, d, J=2.47 Hz), 8.31 (1H, dd, J=4.67, 1.65 Hz), 8.42 (1H, dd, J=7.96, 1.37 Hz), 8.76 (1H, d, J=1.37 Hz), 12.01 (1H, br s); ESIMS found for C₁₅H₁₁FN₄ m/z 267.0 (M+1).

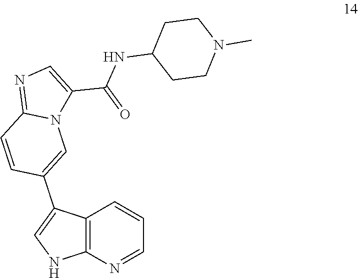 [SMILES: CN1CCC(NC(=O)c2cnc3ccc(-c4c[nH]c5ncccc45)cn23)CC1]

### N-(1-Methylpiperidin-4-yl)-6-(1H-pyrrolo[2,3-b]pyridin-3-yl)imidazo[1,2-a]pyridine-3-carboxamide 14

White solid (46 mg, 0.123 mmol, 27.6% yield). ¹H NMR (499 MHz, DMSO-d₆) δ ppm 1.60 (2H, qd, J=12.05, 3.83 Hz), 1.78-1.86 (2H, m), 1.92-2.02 (2H, m), 2.18 (3H, s), 2.80 (2H, br d, J=11.77 Hz), 3.78-3.90 (1H, m), 7.27 (1H, dd, J=7.94, 4.65 Hz), 7.76 (1H, d, J=9.31 Hz), 7.88 (1H, dd, J=9.31, 1.92 Hz), 8.07 (1H, d, J=2.74 Hz), 8.26 (1H, br d, J=1.37 Hz), 8.27 (1H, dd, J=4.52, 3.15 Hz), 8.33 (1H, dd, J=4.65, 1.37 Hz), 8.38 (1H, s), 9.94 (1H, s), 12.08 (1H, br s); ESIMS found for C₂₁H₂₂N₆O m/z 375.2 (M+1).

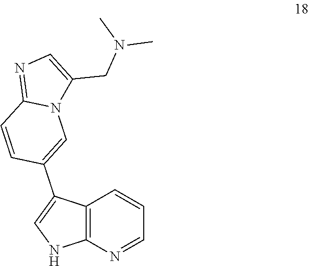 [SMILES: CN(C)Cc1cnc2ccc(-c3c[nH]c4ncccc34)cn12]

### 1-(6-(1H-Pyrrolo[2,3-b]pyridin-3-yl)imidazo[1,2-a]pyridin-3-yl)-N,N-dimethylmethanamine 18

Beige solid (35 mg, 0.120 mmol, 69.1% yield). ¹H NMR (499 MHz, DMSO-d₆) δ ppm 2.21 (6H, s), 3.82 (2H, s), 7.21 (1H, dd, J=7.96, 4.67 Hz), 7.48 (1H, s), 7.59-7.68 (2H, m), 7.97 (1H, d, J=2.47 Hz), 8.29 (1H, d, J=7.96 Hz), 8.30-8.32 (1H, m), 8.68 (1H, s), 12.00 (1H, br s); ESIMS found for C₁₇H₁₇N₅ m/z 292.05 (M+1).

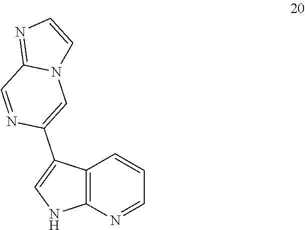 [SMILES: c1cnc2[nH]cc(-c3cn4ccnc4cn3)c2c1]

### 6-(1H-Pyrrolo[2,3-b]pyridin-3-yl)imidazo[1,2-a]pyrazine 20

Beige solid (40 mg, 0.170 mmol, 63.8% yield). ¹H NMR (499 MHz, DMSO-d₆) δ ppm 7.21 (1H, dd, J=8.08, 4.52 Hz), 7.81 (1H, d, J=0.82 Hz), 8.09 (1H, s), 8.12 (1H, s), 8.31 (1H, dd, J=4.52, 1.51 Hz), 8.60 (1H, dd, J=7.94, 1.64 Hz), 9.08 (1H, d, J=1.37 Hz), 9.13 (1H, d, J=0.82 Hz), 12.02 (1H, br s); ESIMS found for C₁₃H₉N₅ m/z 236.1 (M+1).

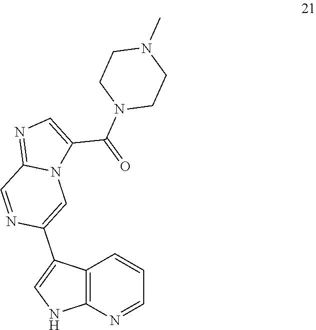 [SMILES: CN1CCN(C(=O)c2cnc3cnc(-c4c[nH]c5ncccc45)cn23)CC1]

### (6-(1H-Pyrrolo[2,3-b]pyridin-3-yl)imidazo[1,2-a]pyrazin-3-yl)(4-methylpiperazin-1-yl)methanone 21

Beige solid (7 mg, 0.019 mmol, 3.9% yield). ¹H NMR (499 MHz, DMSO-d₆) δ ppm 2.23 (3H, s), 2.42 (4H, t, J=4.93 Hz), 3.78 (4H, br s), 7.26 (1H, dd, J=7.94, 4.65 Hz), 8.17 (1H, s), 8.19 (1H, s), 8.26 (1H, dd, J=8.08, 1.51 Hz), 8.35 (1H, dd, J=4.65, 1.37 Hz), 9.14 (1H, d, J=2.46 Hz), 9.51 (1H, d, J=2.46 Hz), 12.20 (1H, br s); ESIMS found for C₁₉H₁₉N₇O m/z 362.15 (M+1).

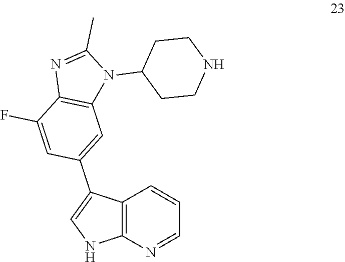 [SMILES: Cc1nc2c(F)cc(-c3c[nH]c4ncccc34)cc2n1C1CCNCC1]

### 4-Fluoro-2-methyl-1-(piperidin-4-yl)-6-(1H-pyrrolo[2,3-b]pyridin-3-yl)-1H-benzo[d]imidazole 23

White solid (45 mg, 0.129 mmol, 52.6% yield). ¹H NMR (499 MHz, DMSO-d₆) δ ppm 1.81 (2H, br d, J=9.61 Hz), 2.27 (2H, qd, J=12.17, 3.84 Hz), 2.61 (3H, s), 2.63-2.72 (2H, m), 3.11 (2H, br d, J=12.08 Hz), 4.37-4.48 (1H, m), 7.19 (1H, dd, J=7.96, 4.67 Hz), 7.27 (1H, d, J=12.08 Hz), 7.71 (1H, d, J=1.10 Hz), 7.91 (1H, d, J=1.92 Hz), 8.24 (1H, dd, J=7.96, 1.37 Hz), 8.29 (1H, dd, J=4.53, 1.51 Hz), 11.94 (1H, br s); ESIMS found for C₂₀H₂₀FN₅ m/z 350.15 (M+1).

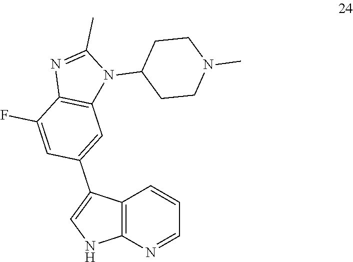 [SMILES: Cc1nc2c(F)cc(-c3c[nH]c4ncccc34)cc2n1C1CCN(C)CC1]

### 4-Fluoro-2-methyl-1-(1-methylpiperidin-4-yl)-6-(1H-pyrrolo[2,3-b]pyridin-3-yl)-1H-benzo[d]imidazole 24

Beige solid (25 mg, 0.069 mmol, 22.4% yield). ¹H NMR (499 MHz, DMSO-d₆) δ ppm 1.87 (2H, br d, J=10.15 Hz), 2.11-2.20 (2H, m), 2.25 (3H, s), 2.43 (2H, qd, J=12.35, 3.57 Hz), 2.61 (3H, s), 2.95 (2H, br d, J=11.80 Hz), 4.33 (1H, tt, J=12.28, 4.19 Hz), 7.19 (1H, dd, J=7.96, 4.67 Hz), 7.27 (1H, dd, J=12.08, 0.82 Hz), 7.61 (1H, d, J=1.10 Hz), 7.93 (1H, d, J=1.92 Hz), 8.22 (1H, dd, J=7.96, 1.37 Hz), 8.29 (1H, dd, J=4.53, 1.51 Hz), 11.94 (1H, br s); ESIMS found for C₂₁H₂₂FN₅ m/z 364.2 (M+1).

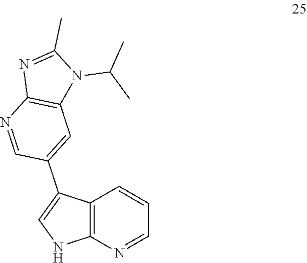 [SMILES: Cc1nc2ncc(-c3c[nH]c4ncccc34)cc2n1C(C)C]

### 1-Isopropyl-2-methyl-6-(1H-pyrrolo[2,3-b]pyridin-3-yl)-1H-imidazo[4,5-b]pyridine 25

Beige solid (36 mg, 0.124 mmol, 55.0% yield). ¹H NMR (499 MHz, DMSO-d₆) δ ppm 1.62 (6H, d, J=6.86 Hz), 2.63 (3H, s), 4.82 (1H, spt, J=6.86 Hz), 7.19 (1H, dd, J=7.96, 4.67 Hz), 7.99 (1H, d, J=2.47 Hz), 8.21 (1H, d, J=1.92 Hz), 8.27 (1H, dd, J=7.96, 1.37 Hz), 8.30 (1H, dd, J=4.67, 1.65 Hz), 8.67 (1H, d, J=1.92 Hz), 11.97 (1H, br s); ESIMS found for C₁₇H₁₇N₅ m/z 292.15 (M+1).

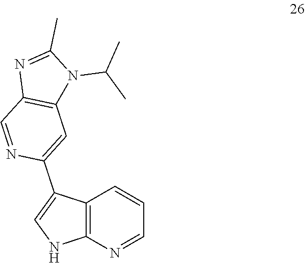 [SMILES: Cc1nc2cnc(-c3c[nH]c4ncccc34)cc2n1C(C)C]

### 3-Isopropyl-2-methyl-5-(1H-pyrrolo[2,3-b]pyridin-3-yl)-3H-imidazo [4,5-b]pyridine 26

White solid (17 mg, 0.058 mmol, 19.8% yield). ¹H NMR (499 MHz, DMSO-d₆) δ ppm 1.63 (6H, d, J=6.86 Hz), 2.60 (3H, s), 4.79 (1H, spt, J=6.91 Hz), 7.16 (1H, dd, J=7.96, 4.67 Hz), 8.03 (1H, d, J=1.10 Hz), 8.25 (1H, dd, J=4.67, 1.65 Hz), 8.30 (1H, d, J=2.74 Hz), 8.80 (1H, dd, J=7.96, 1.65 Hz), 8.82 (1H, d, J=0.82 Hz), 11.88 (1H, br s); ESIMS found for C₁₇H₁₇N₅ m/z 292.1 (M+1).

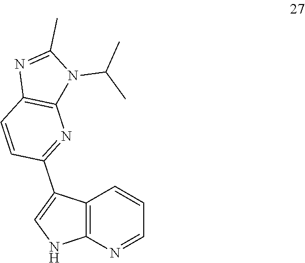 [SMILES: Cc1nc2ccc(-c3c[nH]c4ncccc34)nc2n1C(C)C]

### 3-Isopropyl-2-methyl-5-(1H-pyrrolo[2,3-b]pyridin-3-yl)-3H-imidazo [4,5-b]pyridine 27

White solid (35 mg, 0.120 mmol, 51.8% yield). ¹H NMR (499 MHz, DMSO-d₆) δ ppm 1.76 (6H, d, J=6.59 Hz), 2.60 (3H, s), 4.85 (1H, spt, J=6.91 Hz), 7.22 (1H, dd, J=7.68, 4.67 Hz), 7.73 (1H, d, J=8.51 Hz), 7.87 (1H, d, J=8.51 Hz), 8.23 (1H, s), 8.29 (1H, dd, J=4.53, 1.51 Hz), 8.87 (1H, dd, J=7.68, 1.65 Hz), 11.98 (1H, br s); ESIMS found for C₁₇H₁₇N₅ m/z 292.15 (M+1).

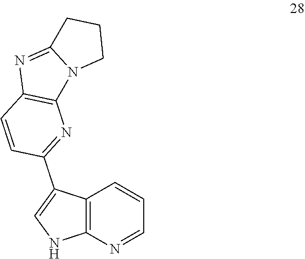 [SMILES: c1cnc2[nH]cc(-c3ccc4nc5n(c4n3)CCC5)c2c1]

### 2-(1H-Pyrrolo[2,3-b]pyridin-3-yl)-7,8-dihydro-6H-pyrrolo [2′,1′:2,3]imidazo[4,5-b]pyridine 28

White solid (46 mg, 0.167 mmol, 53.4% yield). ¹H NMR (499 MHz, DMSO-d₆) δ ppm 2.68 (2H, quin, J=7.34 Hz), 2.98-3.04 (2H, m), 4.24 (2H, t, J=7.00 Hz), 7.19 (1H, dd, J=7.82, 4.53 Hz), 7.73 (1H, d, J=8.51 Hz), 7.90 (1H, d, J=8.51 Hz), 8.21 (1H, d, J=2.47 Hz), 8.28 (1H, dd, J=4.67, 1.65 Hz), 8.91 (1H, dd, J=7.96, 1.65 Hz), 11.95 (1H, br s); ESIMS found for C₁₆H₁₃N₅ m/z 276.1 (M+1).

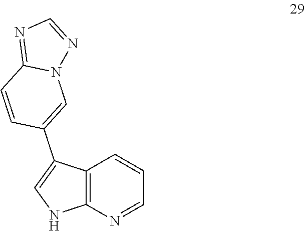 [SMILES: c1cnc2[nH]cc(-c3ccc4ncnn4c3)c2c1]

### 6-(1H-Pyrrolo[2,3-b]pyridin-3-yl)-[1,2,4]triazolo[1,5-a]pyridine 29

Light brown solid (20.0 mg, 0.085 mmol, 20.8% yield). ¹H NMR (500 MHz, DMSO-d₆) δ ppm 1.52-1.61 (4H, m), 1.62-1.70 (2H, m), 3.60-3.69 (4H, m), 7.27 (1H, dd, J=7.96, 4.67 Hz), 7.47 (1H, dd, J=7.27, 2.06 Hz), 8.16 (1H, d, J=1.37 Hz), 8.19 (1H, s), 8.22 (1H, d, J=2.74 Hz), 8.30-8.35 (2H, m), 8.72-8.81 (1H, m), 12.21 (1H, br s); ESIMS found for C₁₃H₉N₅ m/z 236.0 (M+1).

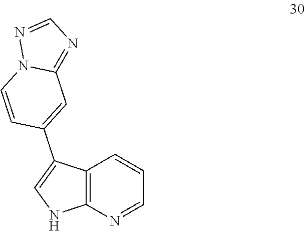 [SMILES: c1cnc2[nH]cc(-c3ccn4ncnc4c3)c2c1]

### 7-(1H-Pyrrolo[2,3-b]pyridin-3-yl)-[1,2,4]triazolo[1,5-a]pyridine 30

Beige solid (25.0 mg, 0.106 mmol, 25.9% yield). ¹H NMR (499 MHz, DMSO-d₆) δ ppm 7.23 (1H, dd, J=7.96, 4.67 Hz), 7.64 (1H, dd, J=7.14, 1.92 Hz), 8.10 (1H, d, J=1.37 Hz), 8.29 (1H, s), 8.33 (1H, dd, J=4.53, 1.51 Hz), 8.45 (1H, s), 8.50 (1H, dd, J=7.96, 1.37 Hz), 8.93 (1H, d, J=7.14 Hz), 12.26 (1H, br s); ESIMS found for C₁₃H₉N₅ m/z 236.0 (M+1).

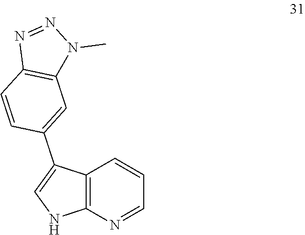 [SMILES: Cn1nnc2ccc(-c3c[nH]c4ncccc34)cc21]

### 1-Methyl-6-(1H-pyrrolo[2,3-b]pyridin-3-yl)-1H-benzo[d][1,2,3]triazole 31

Beige solid (60 mg, 0.241 mmol, 42.6% yield). ¹H NMR (499 MHz, DMSO-d₆) δ ppm 4.36 (3H, s), 7.22 (1H, dd, J=7.96, 4.67 Hz), 7.80 (1H, dd, J=8.64, 1.51 Hz), 8.05 (2H, d, J=8.51 Hz), 8.08 (1H, s), 8.32 (1H, dd, J=4.53, 1.51 Hz), 8.51 (1H, dd, J=7.96, 1.37 Hz), 12.05 (1H, br s); ESIMS found for C₁₄H₁₁N₅ m/z 250.05 (M+1).

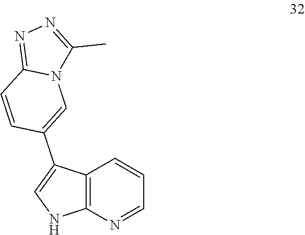 [SMILES: Cc1nnc2ccc(-c3c[nH]c4ncccc34)cn12]

### 3-Methyl-6-(1H-pyrrolo[2,3-b]pyridin-3-yl)-[1,2,4]triazolo[4,3-a] pyridine 32

Beige solid (35 mg, 0.140 mmol, 60.8% yield). ¹H NMR (500 MHz, DMSO-d₆) δ ppm 2.77 (3H, s), 7.21 (1H, dd, J=7.96, 4.67 Hz), 7.74-7.82 (2H, m), 8.05 (1H, s), 8.32 (1H, dd, J=4.67, 1.65 Hz), 8.42 (1H, dd, J=7.96, 1.37 Hz), 8.47 (1H, t, J=1.23 Hz), 12.07 (1H, br s); ESIMS found for C₁₄H₁₁N₅ m/z 250.1 (M+1).

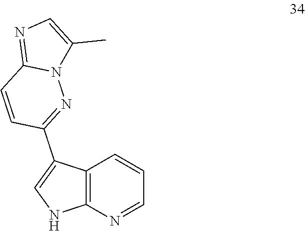 [SMILES: Cc1cnc2ccc(-c3c[nH]c4ncccc34)nn12]

### 3-Methyl-6-(1H-pyrrolo[2,3-b]pyridin-3-yl)imidazo[1,2-b]pyridazine 34

Beige solid (5 mg, 0.020 mmol, 10.3% yield). ¹H NMR (500 MHz, DMSO-d₆) δ ppm 2.63 (3H, s), 7.28 (1H, dd, J=7.96, 4.67 Hz), 7.52 (1H, d, J=0.82 Hz), 7.78 (1H, d, J=9.61 Hz), 8.04 (1H, d, J=9.61 Hz), 8.35 (1H, dd, J=4.67, 1.65 Hz), 8.49 (1H, s), 8.84 (1H, dd, J=7.96, 1.65 Hz), 12.27 (1H, br s); ESIMS found for C₁₄H₁₁N₅ m/z 250.1 (M+1).

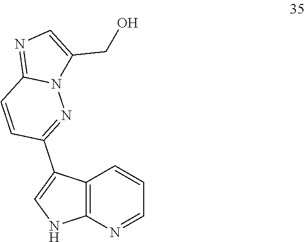 [SMILES: OCc1cnc2ccc(-c3c[nH]c4ncccc34)nn12]

### (6-(1H-Pyrrolo[2,3-b]pyridin-3-yl)imidazo[1,2-b]pyridazin-3-yl) methanol 35

Light rose solid (12 mg, 0.045 mmol, 61.1% yield). ¹H NMR (499 MHz, DMSO-d₆) δ ppm 4.96 (2H, d, J=3.29 Hz), 5.29 (1H, br s), 7.27 (1H, dd, J=7.96, 4.67 Hz), 7.64 (1H, s), 7.82 (1H, d, J=9.61 Hz), 8.07 (1H, d, J=9.61 Hz), 8.34 (1H, dd, J=4.67, 1.65 Hz), 8.50 (1H, s), 8.86 (1H, dd, J=7.96, 1.65 Hz), 12.27 (1H, br s); ESIMS found for C₁₄H₁₁N₅O m/z 266.0 (M+1).

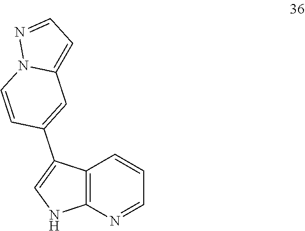 [SMILES: c1cnc2[nH]cc(-c3ccn4nccc4c3)c2c1]

### 3-(Pyrazolo[1,5-a]pyridin-5-yl)-1H-pyrrolo[2,3-b]pyridine 36

Dark yellow solid (13 mg, 0.056 mmol, 51.9% yield). ¹H NMR (500 MHz, DMSO-d₆) δ ppm 6.58 (1H, d, J=2.20 Hz), 7.21 (1H, dd, J=7.96, 4.67 Hz), 7.30 (1H, dd, J=7.41, 1.92 Hz), 7.96 (1H, d, J=2.20 Hz), 8.03 (1H, d, J=1.37 Hz), 8.12 (1H, s), 8.31 (1H, dd, J=4.53, 1.51 Hz), 8.47 (1H, dd, J=7.96, 1.65 Hz), 8.67 (1H, d, J=7.41 Hz), 12.08 (1H, br s); ESIMS found for C₁₄H₁₀N₄ m/z 235.1 (M+1).

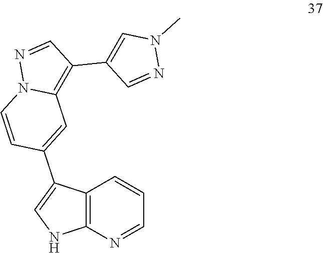 [SMILES: Cn1cc(-c2cnn3ccc(-c4c[nH]c5ncccc45)cc23)cn1]

### 3-(3-(1-Methyl-1H-pyrazol-4-yl)pyrazolo[1,5-a]pyridin-5-yl)-1H-pyrrolo[2,3-b]pyridine 37

Light orange solid (12 mg, 0.038 mmol, 24.8% yield). ¹H NMR (500 MHz, DMSO-d₆) δ ppm 3.92 (3H, s), 7.24 (1H, dd, J=7.96, 4.67 Hz), 7.31 (1H, dd, J=7.14, 1.92 Hz), 7.89 (1H, s), 7.96 (1H, dd, J=1.92, 0.82 Hz), 8.18 (3H, s), 8.32 (1H, dd, J=4.67, 1.65 Hz), 8.39 (1H, dd, J=7.96, 0.82 Hz), 8.68 (1H, dd, J=7.14, 0.82 Hz), 12.13 (1H, br s); ESIMS found for C₁₈H₁₄N₆ m/z 315.05 (M+1).

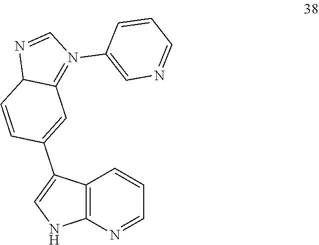

### 3-(3-(Pyridin-3-yl)pyrazolo[1,5-a]pyridin-5-yl)-1H-pyrrolo[2,3-b] pyridine 38

Light yellow solid (14 mg, 0.045 mmol, 33.8% yield). ¹H NMR (500 MHz, DMSO-d₆) δ ppm 7.24 (1H, dd, J=7.96, 4.67 Hz), 7.42 (1H, dd, J=7.41, 1.92 Hz), 7.50 (1H, ddd, J=7.96, 4.67, 0.82 Hz), 8.13 (1H, dd, J=1.92, 0.82 Hz), 8.18 (1H, ddd, J=7.96, 2.47, 1.65 Hz), 8.24 (1H, d, J=2.47 Hz), 8.32 (1H, dd, J=4.67, 1.37 Hz), 8.38 (1H, dd, J=8.10, 1.51 Hz), 8.46 (1H, s), 8.49 (1H, dd, J=4.67, 1.65 Hz), 8.75-8.83 (1H, m), 9.01 (1H, dd, J=2.47, 0.82 Hz), 12.17 (1H, br s); ESIMS found for C₁₉H₁₃N₅ m/z 312.1 (M+1).

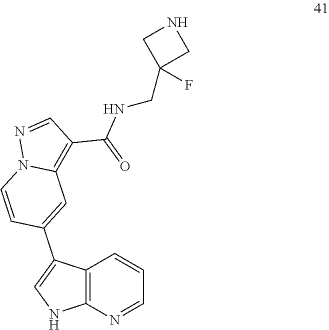 [SMILES: O=C(NCC1(F)CNC1)c1cnn2ccc(-c3c[nH]c4ncccc34)cc12]

### N-((3-Fluoroazetidin-3-yl)methyl)-5-(1H-pyrrolo[2,3-b]pyridin-3-yl) pyrazolo[1,5-a]pyridine-3-carboxamide 41

Light brown solid (47 mg, 0.129 mmol, 55.5% yield). ¹H NMR (499 MHz, DMSO-d₆) δ ppm 3.49-3.64 (4H, m), 3.80 (2H, dd, J=23.30, 6.00 Hz), 7.29 (1H, dd, J=7.80, 4.79 Hz), 7.50 (1H, dd, J=7.39, 1.92 Hz), 8.24 (1H, s), 8.34-8.37 (1H, m), 8.39 (1H, brt, J=6.02 Hz), 8.57 (1H, d, J=1.37 Hz), 8.61 (1H, s), 8.76 (1H, d, J=7.39 Hz), 12.23 (1H, br s); ESIMS found for C₁₉H₁₇FN₆O m/z 365.15 (M+1).

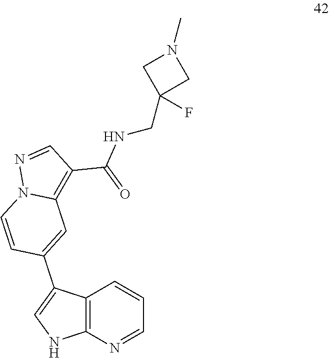 [SMILES: CN1CC(F)(CNC(=O)c2cnn3ccc(-c4c[nH]c5ncccc45)cc23)C1]

### N-((3-Fluoro-1-methylazetidin-3-yl)methyl)-5-(1H-pyrrolo[2,3-b] pyridin-3-yl)pyrazolo[1,5-a]pyridine-3-carboxamide 42

Beige solid (5 mg, 0.013 mmol, 10.2% yield). ¹H NMR (499 MHz, DMSO-d₆) δ ppm 2.29 (3H, s), 3.00-3.11 (2H, m), 3.46-3.56 (2H, m), 3.76 (2H, dd, J=23.30, 6.30 Hz), 7.29 (1H, dd, J=7.80, 4.79 Hz), 7.50 (1H, dd, J=7.26, 2.05 Hz), 8.24 (1H, d, J=2.74 Hz), 8.33-8.37 (2H, m), 8.39-8.43 (1H, m), 8.56 (1H, d, J=1.37 Hz), 8.61 (1H, s), 8.75-8.78 (1H, m), 12.23 (1H, br s); ESIMS found for C₂₀H₁₉FN₆O m/z 379.2 (M+1).

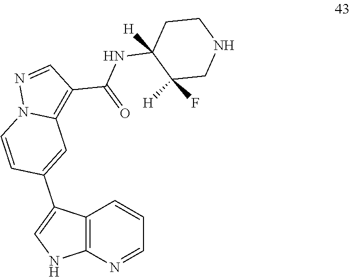 [SMILES: O=C(N[C@@H]1CCNC[C@H]1F)c1cnn2ccc(-c3c[nH]c4ncccc34)cc12]

### N-((3R,4R)-3-Fluoropiperidin-4-yl)-5-(1H-pyrrolo[2,3-b]pyridin-3-yl) pyrazolo[1,5-a]pyridine-3-carboxamide 43

Beige solid (123 mg, 0.325 mmol, 59.6% yield). ¹H NMR (499 MHz, DMSO-d₆) δ ppm 1.46 (1H, qd, J=12.14, 4.11 Hz), 1.83-1.92 (1H, m), 2.43-2.49 (2H, m), 2.83-2.91 (1H, m), 3.22-3.29 (1H, m), 4.03-4.15 (1H, m), 4.43 (1H, dtd, J=50.45, 9.58, 9.58, 4.65 Hz), 7.26-7.33 (1H, m), 7.49 (1H, dd, J=7.26, 2.05 Hz), 8.20 (1H, d, J=8.49 Hz), 8.24 (1H, d, J=2.46 Hz), 8.34 (1H, s), 8.35-8.37 (1H, m), 8.55 (1H, s), 8.57 (1H, d, J=1.64 Hz), 8.75 (1H, d, J=7.39 Hz), 12.22 (1H, br s); ESIMS found for C₂₀H₁₉FN₆O m/z 379.2 (M+1).

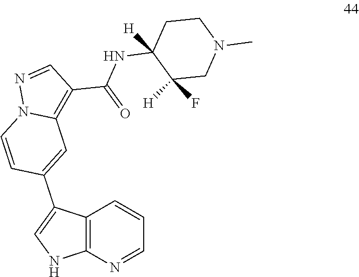 [SMILES: CN1CC[C@@H](NC(=O)c2cnn3ccc(-c4c[nH]c5ncccc45)cc23)[C@H](F)C1]

### N-((3R,4R)-3-Fluoro-1-methylpiperidin-4-yl)-5-(1H-pyrrolo[2,3-b] pyridin-3-yl)pyrazolo[1,5-a]pyridine-3-carboxamide 44

Off-white solid (7 mg, 0.018 mmol, 13.5% yield). ¹H NMR (499 MHz, DMSO-d₆) δ ppm 1.52-1.65 (1H, m), 1.83-1.93 (1H, m), 1.97-2.08 (2H, m), 2.25 (3H, s), 2.69-2.77 (1H, m), 3.10-3.19 (1H, m), 3.94-4.08 (1H, m), 4.57(1H, dtd, J=49.90, 9.65, 9.65, 4.79 Hz), 7.28-7.31 (1H, m), 7.50 (1H, dd, J=7.26, 2.05 Hz), 8.19 (1H, d, J=8.49 Hz), 8.24 (1H, d, J=1.92 Hz), 8.34 (1H, s), 8.35-8.37 (1H, m), 8.53 (1H, s), 8.57 (1H, d, J=1.37 Hz), 8.72-8.80 (1H, m), 12.23 (1H, br s); ESIMS found for C₂₁H₂₁FN₆O m/z 393.2 (M+1).

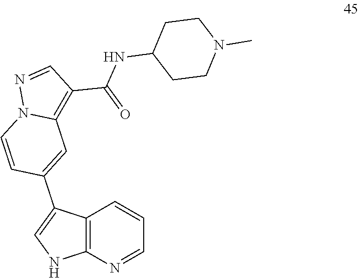 [SMILES: CN1CCC(NC(=O)c2cnn3ccc(-c4c[nH]c5ncccc45)cc23)CC1]

### N-(1-Methylpiperidin-4-yl)-5-(1H-pyrrolo[2,3-b]pyridin-3-yl) pyrazolo[1,5-a]pyridine-3-carboxamide 45

Grey solid (22.0 mg, 0.059 mmol, 14.3% yield). ¹H NMR (500 MHz, DMSO-d₆) δ ppm 1.58 (2H, qd, J=11.98, 3.84 Hz), 1.75-1.85 (2H, m), 1.96 (2H, td, J=11.87, 2.06 Hz), 2.18 (3H, s), 2.80 (2H, br d, J=11.80 Hz), 3.73-3.87 (1H, m), 7.25-7.33(1H, m), 7.47(1H, dd, J=7.41, 2.20 Hz), 7.95 (1H, d, J=7.68 Hz), 8.23 (1H, d, J=2.74 Hz), 8.34 (1H, s), 8.35-8.38 (1H, m), 8.56 (1H, s), 8.56-8.58 (1H, m), 8.70-8.77 (1H, m), 12.22 (1H, br s); ESIMS found for C₂₁H₂₂N₆O m/z 375.1 (M+1).

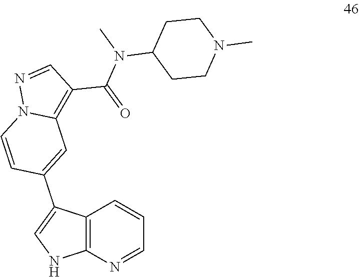 [SMILES: CN1CCC(N(C)C(=O)c2cnn3ccc(-c4c[nH]c5ncccc45)cc23)CC1]

### N-Methyl-N-(1-methylpiperidin-4-yl)-5-(1H-pyrrolo[2,3-b]pyridin-3-yl)pyrazolo[1,5-a]pyridine-3-carboxamide 46

Amber solid (55 mg, 0.142 mmol, 34.3% yield). ¹H NMR (499 MHz, DMSO-d₆) δ ppm 1.64 (2H, br d, J=10.13 Hz), 1.85 (2H, qd, J=12.00, 3.42 Hz), 1.92-2.01 (2H, m), 2.17 (3H, s), 2.85 (2H, br d, J=11.50 Hz), 3.05 (3H, br s), 4.17-4.31 (1H, m), 7.28 (1H, dd, J=7.94, 4.65 Hz), 7.49 (1H, dd, J=7.26, 2.05 Hz), 8.22 (1H, d, J=2.46 Hz), 8.25 (1H, s), 8.30-8.32 (2H, m), 8.34 (1H, dd, J=4.65, 1.64 Hz), 8.74-8.80 (1H, m), 12.21 (1H, br s); ESIMS found for C₂₂H₂₄N₆O m/z 389.2 (M+1).

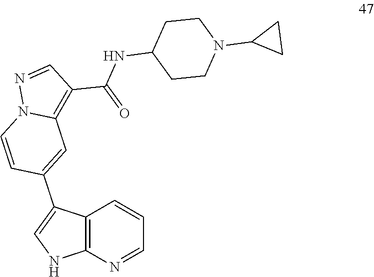 [SMILES: O=C(NC1CCN(C2CC2)CC1)c1cnn2ccc(-c3c[nH]c4ncccc34)cc12]

### N-(1-Cyclopropylpiperidin-4-yl)-5-(1H-pyrrolo[2,3-b]pyridin-3-yl) pyrazolo[1,5-a]pyridine-3-carboxamide 47

Off-white solid (21.0 mg, 0.052 mmol, 20.1% yield). ¹H NMR (499 MHz, DMSO-d₆) δ ppm 0.26-0.34 (2H, m), 0.39-0.46 (2H, m), 1.48 (2H, qd, J=11.91, 3.70 Hz), 1.61 (1H, tt, J=6.71, 3.42 Hz), 1.78-1.86 (2H, m), 2.24 (2H, td, J=11.77, 2.19 Hz), 2.96 (2H, br d, J=11.77 Hz), 3.76-3.89 (1H, m), 7.26-7.33 (1H, m), 7.47 (1H, dd, J=7.26, 2.05 Hz), 7.92 (1H, d, J=7.67 Hz), 8.23 (1H, s), 8.34 (1H, s), 8.35 (1H, q, J=1.46 Hz), 8.54 (1H, s), 8.56-8.57 (1H, m), 8.70-8.78 (1H, m), 12.22 (1H, s); ESIMS found for C₂₃H₂₄N₆O m/z 401.1 (M+1).

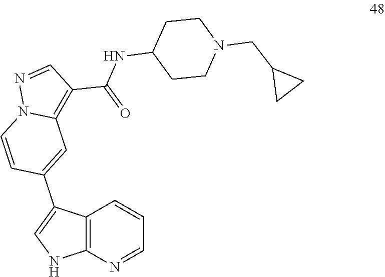 [SMILES: O=C(NC1CCN(CC2CC2)CC1)c1cnn2ccc(-c3c[nH]c4ncccc34)cc12]

### N-(1-(cyclopropylmethyl)piperidin-4-yl)-5-(1H-pyrrolo[2,3-b]pyridin-3-yl)pyrazolo[1,5-a]pyridine-3-carboxamide 48

Beige solid (92 mg, 0.222 mmol, 53.3% yield). ¹H NMR (499 MHz, DMSO-d₆) δ ppm 0.04-0.11(2H, m), 0.42-0.50 (2H, m), 0.77-0.89 (1H, m), 1.57 (2H, qd, J=11.91, 3.70 Hz), 1.78-1.88 (2H, m), 1.94-2.06 (2H, m), 2.18 (2H, d, J=6.57 Hz), 3.00 (2H, br d, J=11.77 Hz), 3.75-3.86 (1H, m), 7.27-7.32 (1H, m), 7.47 (1H, dd, J=7.26, 2.05 Hz), 7.96 (1H, d, J=7.67 Hz), 8.23 (1H, s), 8.34 (1H, s), 8.35-8.36 (1H, m), 8.56 (1H, s), 8.57 (1H, d, J=1.64 Hz), 8.74 (1H, d, J=7.39 Hz), 12.22 (1H, br s); ESIMS found for C₂₄H₂₆N₆O m/z 415.2 (M+1).

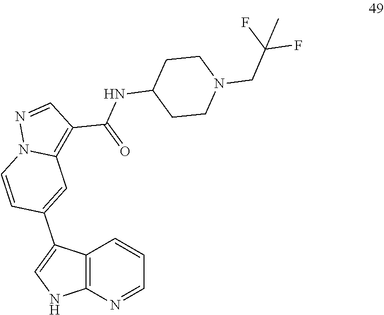 [SMILES: CC(F)(F)CN1CCC(NC(=O)c2cnn3ccc(-c4c[nH]c5ncccc45)cc23)CC1]

### N-(1-(2,2-Difluoropropyl)piperidin-4-yl)-5-(1H-pyrrolo[2,3-b]pyridin-3-yl)pyrazolo[1,5-a]pyridine-3-carboxamide 49

Grey solid (45 mg, 0.103 mmol, 24.8% yield). ¹H NMR (499 MHz, DMSO-d₆) δ ppm 1.54-1.63 (2H, m), 1.63 (3H, br t, J=19.03 Hz), 1.78-1.85 (2H, m), 2.25-2.35 (2H, m), 2.73 (2H, t, J=14.10 Hz), 2.93 (2H, br d, J=12.05 Hz), 3.76-3.87 (1H, m), 7.25-7.32 (1H, m), 7.48 (1H, dd, J=7.39, 2.19 Hz), 7.95 (1H, d, J=7.94 Hz), 8.23 (1H, s), 8.34 (1H, s), 8.35 (1H, s), 8.55 (1H, s), 8.57 (1H, d, J=1.37 Hz), 8.74 (1H, d, J=7.39 Hz), 12.22 (1H, br s); ESIMS found for C₂₃H₂₄F₂N₆O m/z 439.2 (M+1).

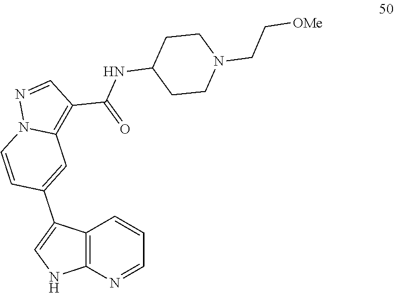 [SMILES: COCCN1CCC(NC(=O)c2cnn3ccc(-c4c[nH]c5ncccc45)cc23)CC1]

### N-(1-(2-Methoxyethyl)piperidin-4-yl)-5-(1H-pyrrolo[2,3-b]pyridin-3-yl)pyrazolo[1,5-a]pyridine-3-carboxamide 50

White solid (42 mg, 0.100 mmol, 24.6% yield). ¹H NMR (499 MHz, DMSO-d₆) δ ppm 1.55 (2H, qd, J=11.96, 3.56 Hz), 1.81 (2H, br d, J=9.86 Hz), 2.00-2.11 (2H, m), 2.48 (2H, t, J=6.05 Hz), 2.91 (2H, br d, J=11.77 Hz), 3.24 (3H, s), 3.44 (2H, t, J=5.89 Hz), 3.74-3.86 (1H, m), 7.26-7.32 (1H, m), 7.47 (1H, dd, J=7.26, 2.05 Hz), 7.94 (1H, d, J=7.67 Hz), 8.23 (1H, s), 8.34 (1H, s), 8.35 (1H, d, J=1.10 Hz), 8.55 (1H, s), 8.56 (1H, d, J=1.64 Hz), 8.74 (1H, d, J=7.12 Hz), 12.22 (1H, br s); ESIMS found for C₂₃H₂₆N₆O₂ m/z 419.2 (M+1).

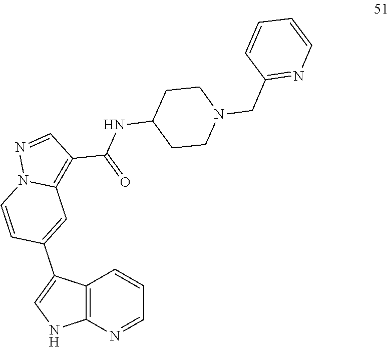 [SMILES: O=C(NC1CCN(Cc2ccccn2)CC1)c1cnn2ccc(-c3c[nH]c4ncccc34)cc12]

### N-(1-(Pyridin-2-ylmethyl)piperidin-4-yl)-5-(1H-pyrrolo[2,3-b]pyridin-3-yl)pyrazolo[1,5-a]pyridine-3-carboxamide 51

White solid (36 mg, 0.080 mmol, 22.0% yield). ¹H NMR (499 MHz, DMSO-d₆) δ ppm 1.61 (2H, qd, J=11.96, 3.83 Hz), 1.80-1.89 (2H, m), 2.08-2.19 (2H, m), 2.87 (2H, br d, J=11.77 Hz), 3.61 (2H, s), 3.79-3.91 (1H, m), 7.25-7.31 (2H, m), 7.45 (1H, d, J=7.67 Hz), 7.48 (1H, dd, J=7.26, 2.05 Hz), 7.78 (1H, td, J=7.67, 1.92 Hz), 7.97 (1H, d, J=7.94 Hz), 8.23 (1H, d, J=2.46 Hz), 8.34 (1H, s), 8.35 (1H, s), 8.47-8.52 (1H, m), 8.56 (1H, s), 8.57 (1H, d, J=1.37 Hz), 8.74 (1H, d, J=7.39 Hz), 12.22 (1H, br s); ESIMS found for C₂₆H₂₅N₇O m/z 452.2 (M+1).

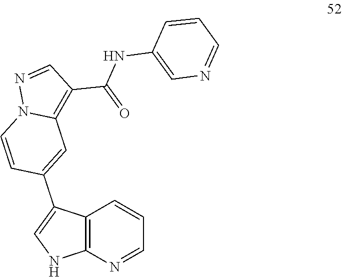 [SMILES: O=C(Nc1cccnc1)c1cnn2ccc(-c3c[nH]c4ncccc34)cc12]

### N-(Pyridin-3-yl)-5-(1H-pyrrolo[2,3-b]pyridin-3-yl)pyrazolo[1,5-a]pyridine-3-carboxamide 52

Brown solid (5.0 mg, 0.014 mmol, 5.3% yield). ¹H NMR (500 MHz, DMSO-d₆) δ ppm 7.31 (1H, dd, J=7.96, 4.67 Hz), 7.40 (1H, dd, J=8.23, 4.39 Hz), 7.54-7.55 (1H, m), 7.57 (1H, dd, J=7.27, 2.06 Hz), 8.23 (1H, ddd, J=8.37, 2.47, 1.51 Hz), 8.27-8.32 (2H, m), 8.36 (1H, dd, J=4.67, 1.65 Hz), 8.38 (1H, dd, J=8.10, 1.51 Hz), 8.60 (1H, d, J=1.37 Hz), 8.77 (1H, s), 8.84 (1H, d, J=7.14 Hz), 8.93 (1H, d, J=2.47 Hz), 10.12 (1H, s), 12.27 (1H, br s); ESIMS found for C₂₀H₁₄N₆O m/z 355.1 (M+1).

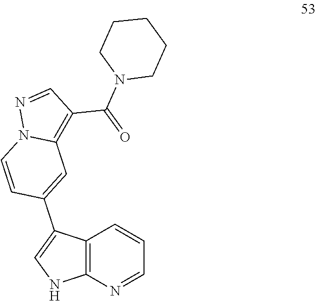 [SMILES: O=C(c1cnn2ccc(-c3c[nH]c4ncccc34)cc12)N1CCCCC1]

### (5-(1H-Pyrrolo[2,3-b]pyridin-3-yl)pyrazolo[1,5-a]pyridin-3-yl) (piperidin-1-yl)methanone 53

Tan solid (28.0 mg, 0.081 mmol, 20.8% yield). ¹H NMR (500 MHz, DMSO-d₆) δ ppm 1.52-1.61 (4H, m), 1.62-1.70 (2H, m), 3.60-3.69 (4H, m), 7.27 (1H, dd, J=7.96, 4.67 Hz), 7.47 (1H, dd, J=7.27, 2.06 Hz), 8.16 (1H, d, J=1.37 Hz), 8.19 (1H, s), 8.22 (1H, d, J=2.74 Hz), 8.30-8.35 (2H, m), 8.72-8.81 (1H, m), 12.21 (1H, br s); ESIMS found for C₂₀H₁₉N₅O m/z 346.1 (M+1).

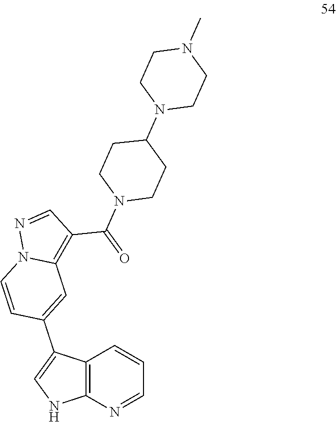 [SMILES: CN1CCN(C2CCN(C(=O)c3cnn4ccc(-c5c[nH]c6ncccc56)cc34)CC2)CC1]

### (5-(1H-Pyrrolo[2,3-b]pyridin-3-yl)pyrazolo[1,5-a]pyridin-3-yl)(4-(4-methylpiperazin-1-yl)piperidin-1-yl)methanone 54

Beige solid (6 mg, 0.014 mmol, 4.9% yield). ¹H NMR (499 MHz, DMSO-d₆) δ ppm 1.36-1.50 (2H, m), 1.83 (2H, br d, J=11.23 Hz), 2.13 (3H, s), 2.21-2.38 (4H, m), 2.41-2.49 (4H, m), 2.90-3.11 (3H, m), 4.28-4.40 (2H, m), 7.26 (1H, dd, J=7.80, 4.79 Hz), 7.48 (1H, dd, J=7.39, 1.92 Hz), 8.15 (1H, d, J=1.37 Hz), 8.21-8.24 (2H, m), 8.31-8.35 (2H, m), 8.77 (1H, d, J=7.12 Hz), 12.22 (1H, br s); ESIMS found for C₂₅H₂₉N₇O m/z 444.3 (M+1).

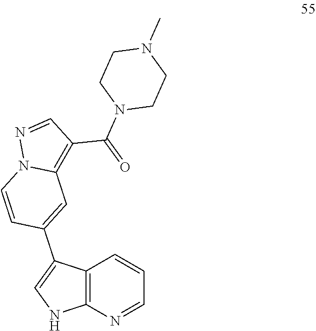 [SMILES: CN1CCN(C(=O)c2cnn3ccc(-c4c[nH]c5ncccc45)cc23)CC1]

### (5-(1H-Pyrrolo[2,3-b]pyridin-3-yl)pyrazolo[1,5-a]pyridin-3-yl)(4-methylpiperazin-1-yl)methanone 55

Yellow solid (52.5 mg, 0.146 mmol, 47.3% yield). ¹H NMR (499 MHz, DMSO-d₆) δ ppm 2.22 (3H, s), 2.38 (4H, t, J=5.08 Hz), 3.66-3.72 (4H, m), 7.28 (1H, dd, J=7.82, 4.80 Hz), 7.49 (1H, dd, J=7.14, 1.92 Hz), 8.15-8.20 (1H, m), 8.22-8.25 (2H, m), 8.32-8.34 (1H, m), 8.34 (1H, s), 8.78 (1H, dd, J=7.41, 0.82 Hz), 12.22 (1H, br s); ESIMS found for C₂₀H₂₀N₆O m/z 361.1 (M+1).

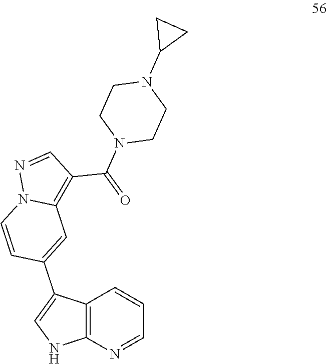 [SMILES: O=C(c1cnn2ccc(-c3c[nH]c4ncccc34)cc12)N1CCN(C2CC2)CC1]

### (5-(1H-Pyrrolo[2,3-b]pyridin-3-yl)pyrazolo[1,5-a]pyridin-3-yl)(4-cyclopropylpiperazin-1-yl)methanone 56

Tan solid (25 mg, 0.065 mmol, 15.5% yield). ¹H NMR (499 MHz, DMSO-d₆) δ ppm 0.31-0.39 (2H, m), 0.41-0.49 (2H, m), 1.66 (1H, tt, J=6.61, 3.52 Hz), 2.61 (4H, t, J=4.93 Hz), 3.62-3.69 (4H, m), 7.24-7.31 (1H, m), 7.49 (1H, dd, J=7.26, 2.05 Hz), 8.18 (1H, d, J=1.64 Hz), 8.23-8.25 (2H, m), 8.32-8.34 (1H, m), 8.34 (1H, s), 8.78 (1H, d, J=7.12 Hz), 12.22 (1H, br s); ESIMS found for C₂₂H₂₂N₆O m/z 387.2 (M+1).

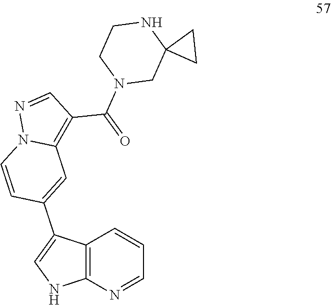 [SMILES: O=C(c1cnn2ccc(-c3c[nH]c4ncccc34)cc12)N1CCNC2(CC2)C1]

### (5-(1H-Pyrrolo[2,3-b]pyridin-3-yl)pyrazolo[1,5-a]pyridin-3-yl)(4,7-diazaspiro[2.5]octan-7-yl)methanone 57

White solid (45 mg, 0.121 mmol, 73.2% yield). ¹H NMR (499 MHz, DMSO-d₆) δ ppm 0.45 (4H, s), 2.78-2.84 (2H, m), 3.53 (2H, s), 3.66 (2H, dd, J=5.63, 4.25 Hz), 7.27 (1H, dd, J=7.96, 4.67 Hz), 7.48 (1H, dd, J=7.27, 2.06 Hz), 8.17 (1H, d, J=1.37 Hz), 8.20 (1H, s), 8.23 (1H, d, J=2.47 Hz), 8.30-8.35 (2H, m), 8.76 (1H, d, J=7.14 Hz), 12.22 (1H, br s); ESIMS found for C₂₁H₂₀N₆O m/z 373.1 (M+1).

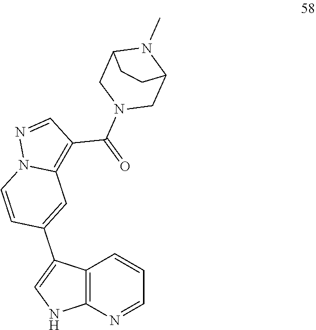 [SMILES: CN1C2CCC1CN(C(=O)c1cnn3ccc(-c4c[nH]c5ncccc45)cc13)C2]

### (5-(1H-Pyrrolo[2,3-b]pyridin-3-yl)pyrazolo[1,5-a]pyridin-3-yl)(8-methyl-3,8-diazabicyclo[3.2.1]octan-3-yl)methanone 58

Off-white solid (10 mg, 0.026 mmol, 23.8% yield). ¹H NMR (499 MHz, DMSO-d₆) δ ppm 1.50 (2H, br d, J=3.56 Hz), 1.89 (2H, br d, J=4.65 Hz), 2.19 (3H, s), 3.12 (2H, br s), 3.17-3.44 (2H, m), 3.92-4.17 (2H, m), 7.28 (1H, dd, J=7.94, 4.65 Hz), 7.48 (1H, dd, J=7.39, 2.19 Hz), 8.21-8.24 (3H, m), 8.31 (1H, dd, J=8.08, 1.51 Hz), 8.34 (1H, dd, J=4.65, 1.64 Hz), 8.77 (1H, dd, J=7.12, 0.82 Hz), 12.22 (1H, br s); ESIMS found for C₂₂H₂₂N₆O m/z 387.1 (M+1).

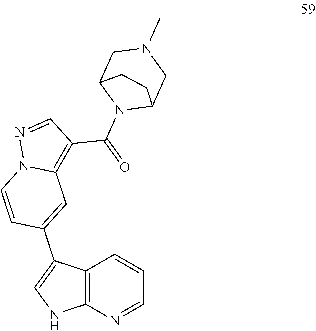 [SMILES: CN1CC2CCC(C1)N2C(=O)c1cnn2ccc(-c3c[nH]c4ncccc34)cc12]

### (5-(1H-Pyrrolo[2,3-b]pyridin-3-yl)pyrazolo[1,5-a]pyridin-3-yl)(3-methyl-3,8-diazabicyclo[3.2.1]octan-8-yl)methanone 59

Off-white solid (41.0 mg, 0.106 mmol, 24.7% yield). ¹H NMR (499 MHz, DMSO-d₆) δ ppm 1.79-1.85 (2H, m), 1.87 (2H, br d, J=7.67 Hz), 2.19 (3H, s), 2.25 (2H, br d, J=9.86 Hz), 2.73 (2H, dd, J=10.68, 1.92 Hz), 4.61 (2H, br s), 7.28 (1H, dd, J=7.94, 4.65 Hz), 7.50 (1H, dd, J=7.26, 2.05 Hz), 8.24 (1H, d, J=3.01 Hz), 8.32 (1H, s), 8.32-8.36 (3H, m), 8.79 (1H, d, J=7.39 Hz), 12.22 (1H, br s); ESIMS found for C₂₂H₂₂N₆O m/z 387.1 (M+1).

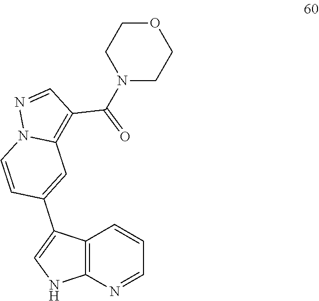 [SMILES: O=C(c1cnn2ccc(-c3c[nH]c4ncccc34)cc12)N1CCOCC1]

### (5-(1H-Pyrrolo[2,3-b]pyridin-3-yl)pyrazolo[1,5-a]pyridin-3-yl) (morpholino)methanone 60

Tan solid (25.0 mg, 0.072 mmol, 18.5% yield). ¹H NMR (500 MHz, DMSO-d₆) δ ppm 3.64-3.69 (4H, m), 3.69-3.73 (4H, m), 7.24-7.32 (1H, m), 7.50 (1H, dd, J=7.27, 2.06 Hz), 8.20-8.22 (1H, m), 8.23 (1H, s), 8.28 (1H, s), 8.32-8.34 (1H, m), 8.34 (1H, s), 8.78 (1H, dd, J=7.41, 0.82 Hz), 12.22 (1H, br s); ESIMS found for C₁₉H₁₇N₅O₂ m/z 348.1 (M+1).

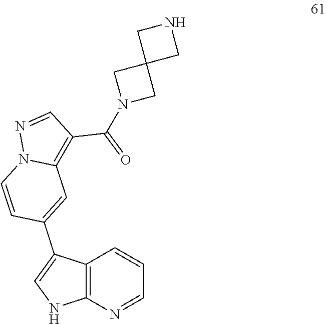 [SMILES: O=C(c1cnn2ccc(-c3c[nH]c4ncccc34)cc12)N1CC2(CNC2)C1]

### (5-(1H-Pyrrolo[2,3-b]pyridin-3-yl)pyrazolo[1,5-a]pyridin-3-yl)(2,6-diazaspiro[3.3]heptan-2-yl)methanone 61

Beige solid (18 mg, 0.050 mmol, 41.1% yield). ¹H NMR (499 MHz, DMSO-d₆) δ ppm 3.62 (4H, s), 4.04-4.25 (2H, m), 4.45-4.64 (2H, m), 7.29 (1H, dd, J=7.94, 4.65 Hz), 7.53 (1H, dd, J=7.26, 2.05 Hz), 8.25 (1H, s), 8.27 (1H, s), 8.31-8.36 (2H, m), 8.57 (1H, d, J=1.37 Hz), 8.76-8.82 (1H, m), 12.19 (1H, br s); ESIMS found for C₂₀H₁₈N₆O m/z 359.2 (M+1).

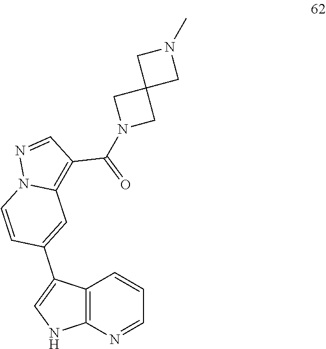 [SMILES: CN1CC2(C1)CN(C(=O)c1cnn3ccc(-c4c[nH]c5ncccc45)cc13)C2]

### (5-(1H-Pyrrolo[2,3-b]pyridin-3-yl)pyrazolo[1,5-a]pyridin-3-yl)(6-methyl-2,6-diazaspiro[3.3]heptan-2-yl)methanone 62

Off-white solid (11 mg, 0.030 mmol, 70.6% yield). ¹H NMR (499 MHz, DMSO-d₆) δ ppm 2.18 (3H, s), 3.27 (4H, s), 4.11 (2H, br s), 4.54 (2H, br s), 7.29 (1H, br dd, J=7.94, 4.65 Hz), 7.53 (1H, dd, J=7.39, 2.19 Hz), 8.25 (1H, d, J=2.46 Hz), 8.26 (1H, s), 8.31-8.36 (2H, m), 8.57 (1H, d, J=1.37 Hz), 8.79 (1H, d, J=7.12 Hz), 12.24 (1H, br s); ESIMS found for C₂₁H₂₀N₆O m/z 373.2 (M+1).

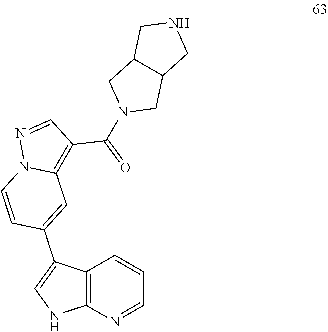 [SMILES: O=C(c1cnn2ccc(-c3c[nH]c4ncccc34)cc12)N1CC2CNCC2C1]

### (5-(1H-Pyrrolo[2,3-b]pyridin-3-yl)pyrazolo[1,5-a]pyridin-3-yl) (hexahydropyrrolo[3,4-c]pyrrol-2(1H)-yl)methanone 63

Light brown solid (47 mg, 0.126 mmol, 70.2% yield). ¹H NMR (499 MHz, DMSO-d₆) δ ppm 2.66 (1H, d, J=3.01 Hz), 2.68 (1H, d, J=3.01 Hz), 2.80 (2H, br s), 2.91 (2H, br dd, J=10.95, 7.12 Hz), 3.49-3.60 (2H, m), 3.89-4.03 (2H, m), 7.29 (1H, dd, J=7.80, 4.79 Hz), 7.51 (1H, dd, J=7.26, 2.05 Hz), 8.23 (1H, s), 8.32-8.34 (1H, m), 8.35 (1H, s), 8.37 (1H, s), 8.55 (1H, d, J=1.64 Hz), 8.77 (1H, d, J=7.12 Hz), 12.22 (1H, br s); ESIMS found for C₂₁H₂₀N₆O m/z 373.2 (M+1).

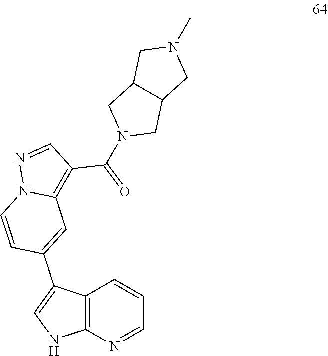 [SMILES: CN1CC2CN(C(=O)c3cnn4ccc(-c5c[nH]c6ncccc56)cc34)CC2C1]

### (5-(1H-Pyrrolo[2,3-b]pyridin-3-yl)pyrazolo[1,5-a]pyridin-3-yl)(5-methylhexahydropyrrolo[3,4-c]pyrrol-2(1H)-yl)methanone 64

White solid (5 mg, 0.013 mmol, 10.3% yield). ¹H NMR (499 MHz, DMSO-d₆) δ ppm 2.22 (3H, s), 2.38-2.46 (2H, m), 2.53 (2H, dd, J=9.03, 1.92 Hz), 2.87 (2H, br s), 3.58 (2H, br d, J=3.29 Hz), 3.97 (2H, br d, J=1.64 Hz), 7.28 (1H, dd, J=7.80, 4.79 Hz), 7.51 (1H, dd, J=7.26, 2.05 Hz), 8.23 (1H, d, J=2.74 Hz), 8.32-8.34 (1H, m), 8.34 (1H, s), 8.36 (1H, s), 8.51-8.56 (1H, m), 8.77 (1H, dd, J=7.12, 0.82 Hz), 12.22 (1H, br s); ESIMS found for C₂₂H₂₂N₆O m/z 387.2 (M+1).

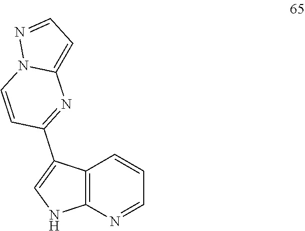 [SMILES: c1cnc2[nH]cc(-c3ccn4nccc4n3)c2c1]

### 5-(1H-Pyrrolo[2,3-b]pyridin-3-yl)pyrazolo[1,5-a]pyrimidine 65

Beige solid (6.5 mg, 0.028 mmol, 94.3% yield). ¹H NMR (500 MHz, DMSO-d₆) δ ppm 6.61 (1H, dd, J=2.20, 0.82 Hz), 7.26 (1H, dd, J=7.96, 4.67 Hz), 7.56 (1H, d, J=7.41 Hz), 8.11 (1H, d, J=2.47 Hz), 8.33 (1H, dd, J=4.67, 1.65 Hz), 8.57 (1H, s), 8.92 (1H, dd, J=7.82, 1.78 Hz), 8.99 (1H, dd, J=7.41, 0.82 Hz), 12.33 (1H, br s); ESIMS found for C₂₂H₂₂N₆O m/z 236.1 (M+1).

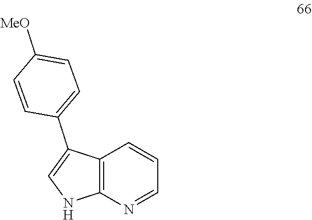 [SMILES: COc1ccc(-c2c[nH]c3ncccc23)cc1]

### 3-(4-Methoxyphenyl)-1H-pyrrolo[2,3-b]pyridine 66

Beige solid (14 mg, 0.062 mmol, 28.4% yield). ¹H NMR (499 MHz, DMSO-d₆) δ ppm 3.79 (3H, s), 6.97-7.06 (2H, m), 7.13 (1H, dd, J=7.96, 4.67 Hz), 7.60-7.67 (2H, m), 7.74 (1H, d, J=2.47 Hz), 8.22 (1H, dd, J=7.96, 1.37 Hz), 8.25 (1H, dd, J=4.53, 1.51 Hz), 11.79 (1H, br s); ESIMS found for C₁₄H₁₂N₂O m/z 225.1 (M+1).

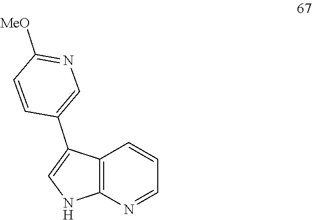 [SMILES: COc1ccc(-c2c[nH]c3ncccc23)cn1]

### 3-(6-Methoxypyridin-3-yl)-1H-pyrrolo[2,3-b]pyridine 67

White solid (20 mg, 0.089 mmol, 59.0% yield). ¹H NMR (499 MHz, DMSO-d₆) δ ppm 3.89 (3H, s), 6.90 (1H, d, J=8.51 Hz), 7.14 (1H, dd, J=7.96, 4.67 Hz), 7.86 (1H, d, J=2.47 Hz), 8.04 (1H, dd, J=8.51, 2.47 Hz), 8.25 (1H, dd, J=7.96, 1.37 Hz), 8.27 (1H, dd, J=4.67, 1.37 Hz), 8.53 (1H, d, J=2.47 Hz), 11.91(1H, br s); ESIMS found for C₁₃H₁₁N₃O m/z 226.1 (M+1).

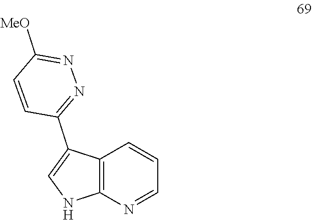 [SMILES: COc1ccc(-c2c[nH]c3ncccc23)nn1]

### 3-(6-Methoxypyridazin-3-yl)-1H-pyrrolo[2,3-b]pyridine 69

White solid (33.5 mg, 0.148 mmol, 38.2% yield). ¹H NMR (499 MHz, DMSO-d₆) δ ppm 4.05 (3H, s), 7.21 (1H, dd, J=7.96, 4.67 Hz), 7.23 (1H, d, J=9.06 Hz), 8.14 (1H, d, J=9.33 Hz), 8.29-8.31 (1H, m), 8.30(1H, s), 8.77(1H, dd, J=7.96, 1.65 Hz), 12.11 (1H, br s); ESIMS found for C₁₂H₁₀N₄O m/z 227.2 (M+1).

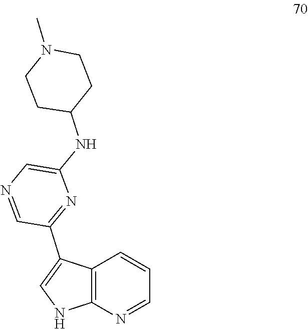 [SMILES: CN1CCC(Nc2cncc(-c3c[nH]c4ncccc34)n2)CC1]

### N-(1-Methylpiperidin-4-yl)-6-(1H-pyrrolo[2,3-b]pyridin-3-yl)pyrazin-2-amine 70

Beige solid (42 mg, 0.136 mmol, 47.0% yield). ¹H NMR (499 MHz, DMSO-d₆) δ ppm 1.46-1.61 (2H, m), 2.01 (2H, br d, J=10.70 Hz), 2.10 (2H, br t, J=10.57 Hz), 2.22 (3H, s), 2.80 (2H, br d, J=11.53 Hz), 3.73-3.86 (1H, m), 6.91 (1H, d, J=7.14 Hz), 7.16 (1H, dd, J=7.96, 4.67 Hz), 7.68 (1H, s), 8.22 (1H, d, J=2.74 Hz), 8.23 (1H, s), 8.27 (1H, dd, J=4.67, 1.65 Hz), 8.70 (1H, dd, J=7.96, 1.65 Hz), 12.05 (1H, br s); ESIMS found for C₁₇H₂₀N₆ m/z 309.1 (M+1).

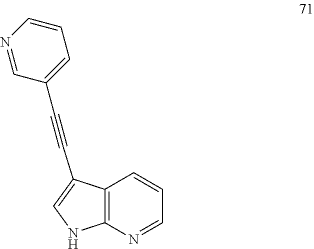 [SMILES: C(#Cc1c[nH]c2ncccc12)c1cccnc1]

### 3-(Pyridin-3-ylethynyl)-1H-pyrrolo[2,3-b]pyridine 71

Beige solid (35 mg, 0.160 mmol, 39.0% yield). ¹H NMR (499 MHz, DMSO-d₆) δ ppm 7.21 (1H, dd, J=7.96, 4.67 Hz), 7.45 (1H, ddd, J=7.89, 4.87, 0.96 Hz), 7.95-7.99 (2H, m), 8.13 (1H, dd, J=7.68, 1.65 Hz), 8.33 (1H, dd, J=4.67, 1.37 Hz), 8.55 (1H, dd, J=4.80, 1.51 Hz), 8.74-8.79 (1H, m), 12.23 (1H, br s); ESIMS found for C₁₄H₉N₃ m/z 220.0 (M+1).

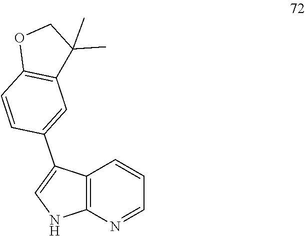 [SMILES: CC1(C)COc2ccc(-c3c[nH]c4ncccc34)cc21]

### 3-(3,3-Dimethyl-2,3-dihydrobenzofuran-5-yl)-1H-pyrrolo[2,3-b] pyridine 72

Beige solid (50 mg, 0.189 mmol, 46.2% yield). ¹H NMR (499 MHz, DMSO-d₆) δ ppm 1.36 (6H, s), 4.24 (2H, s), 6.83 (1H, d, J=8.23 Hz), 7.13 (1H, dd, J=7.82, 4.53 Hz), 7.42 (1H, dd, J=8.23, 1.92 Hz), 7.50 (1H, d, J=1.92 Hz), 7.71 (1H, s), 8.20 (1H, dd, J=7.96, 1.37 Hz), 8.25 (1H, dd, J=4.67, 1.37 Hz), 11.76 (1H, br s); ESIMS found for C₁₇H₁₆N₂O m/z 265.1 (M+1).

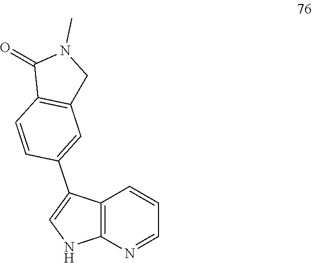 [SMILES: CN1Cc2cc(-c3c[nH]c4ncccc34)ccc2C1=O]

### 2-Methyl-5-(1H-pyrrolo[2,3-b]pyridin-3-yl)isoindolin-1-one 76

White solid (20.1 mg, 0.076 mmol, 33.1% yield). ¹HNMR (499 MHz, DMSO-d₆) δ ppm 3.09 (3H, s), 4.51 (2H, s), 7.19 (1H, dd, J=7.96, 4.67 Hz), 7.69 (1H, d, J=7.96 Hz), 7.84 (1H, dd, J=7.96, 1.10 Hz), 7.94 (1H, s), 8.02 (1H, d, J=2.47 Hz), 8.30 (1H, dd, J=4.67, 1.37 Hz), 8.39 (1H, dd, J=7.96, 1.37 Hz), 12.04 (1H, br s); ESIMS found for C₁₆H₁₃N₃O m/z 264.1 (M+1).

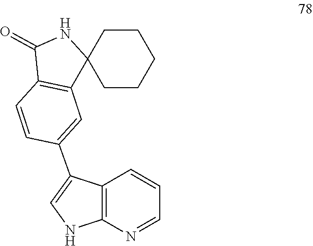 [SMILES: O=C1NC2(CCCCC2)c2cc(-c3c[nH]c4ncccc34)ccc21]

### 6′-(1H-Pyrrolo[2,3-b]pyridin-3-yl)spiro[cyclohexane-1,1′-isoindolin]-3′-one 78

White solid (4.2 mg, 0.013 mmol, 14.1% yield). ¹H NMR (499 MHz, DMSO-d₆) δ ppm 1.41 (3H, br d, J=12.90 Hz), 1.67-1.76 (5H, m), 2.00-2.11 (2H, m), 7.20 (1H, dd, J=7.96, 4.67 Hz), 7.64 (1H, d, J=7.68 Hz), 7.80 (1H, dd, J=7.96, 1.37 Hz), 7.90 (1H, s), 8.03 (1H, d, J=2.74 Hz), 8.30 (1H, dd, J=4.67, 1.37 Hz), 8.36 (1H, dd, J=8.10, 1.24 Hz), 9.07 (1H, s), 12.05 (1H, br s); ESIMS found for C₂₀H₁₉N₃O m/z 318.15 (M+1).

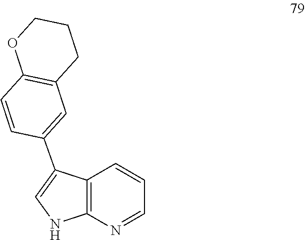 [SMILES: c1cnc2[nH]cc(-c3ccc4c(c3)CCCO4)c2c1]

### 3-(Chroman-6-yl)-1H-pyrrolo[2,3-b]pyridine 79

White solid (50 mg, 0.200 mmol, 86.7% yield). ¹H NMR (499 MHz, DMSO-d₆) δ ppm 1.90-2.00(2H, m), 2.82(2H, t, J=6.45 Hz), 4.11-4.18 (2H, m), 6.79(1H, d, J=8.78 Hz), 7.12 (1H, dd, J=7.96, 4.67 Hz), 7.36-7.40 (2H, m), 7.69 (1H, d, J=2.20 Hz), 8.21 (1H, dd, J=7.96, 1.37 Hz), 8.24 (1H, dd, J=4.53, 1.51 Hz), 11.74 (1H, br s); ESIMS found for C₁₆H₁₄N₂O m/z 251.1 (M+1).

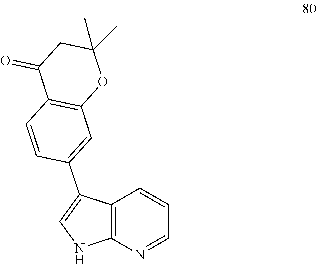 [SMILES: CC1(C)CC(=O)c2ccc(-c3c[nH]c4ncccc34)cc2O1]

### 2,2-Dimethyl-7-(1H-pyrrolo[2,3-b]pyridin-3-yl)chroman-4-one 80

White solid (12 mg, 0.041 mmol, 20.9% yield). ¹H NMR (499 MHz, DMSO-d₆) δ ppm 1.44 (6H, s), 2.79 (2H, s), 7.19 (1H, dd, J=7.96, 4.67 Hz), 7.32 (1H, d, J=1.65 Hz), 7.46 (1H, dd, J=8.23, 1.65 Hz), 7.77 (1H, d, J=8.23 Hz), 8.12 (1H, s), 8.30 (1H, dd, J=4.67, 1.37 Hz), 8.34 (1H, dd, J=8.10, 1.51 Hz), 12.15 (1H, br s); ESIMS found for C₁₈H₁₆N₂O₂ m/z 293.1 (M+1).

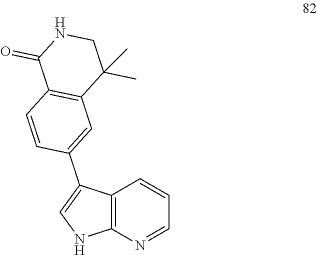 [SMILES: CC1(C)CNC(=O)c2ccc(-c3c[nH]c4ncccc34)cc21]

### 4,4-Dimethyl-6-(1H-pyrrolo[2,3-b]pyridin-3-yl)-3,4-dihydroisoquinolin-1(2H)-one 82

Off-white solid (29 mg, 0.095 mmol, 69.2% yield). ¹H NMR (499 MHz, DMSO-d₆) δ ppm 1.35 (6H, s), 3.20 (2H, d, J=3.02 Hz), 7.19 (1H, dd, J=7.96, 4.67 Hz), 7.68-7.71 (2H, m), 7.89 (1H, br s), 7.89-7.92 (1H, m), 8.04 (1H, d, J=2.20 Hz), 8.28-8.33 (2H, m), 12.05 (1H, br s); ESIMS found for C₁₈H₁₇N₃O m/z 292.1 (M+1).

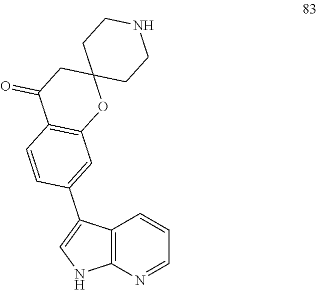 [SMILES: O=C1CC2(CCNCC2)Oc2cc(-c3c[nH]c4ncccc34)ccc21]

### 7-(1H-Pyrrolo[2,3-b]pyridin-3-yl)spiro[chromane-2,4′-piperidin]-4-one 83

Beige solid (74 mg, 0.222 mmol, 87.5% yield). ¹H NMR (499 MHz, DMSO-d₆) δ ppm 1.55-1.66 (2H, m), 1.84 (2H, br d, J=13.45 Hz), 2.69 (2H, dt, J=12.56, 4.15 Hz), 2.78 (2H, s), 2.84-2.94 (2H, m), 7.20 (1H, dd, J=7.96, 4.67 Hz), 7.36 (1H, d, J=1.65 Hz), 7.45 (1H, dd, J=8.23, 1.65 Hz), 7.76 (1H, d, J=8.23 Hz), 8.13 (1H, s), 8.30 (1H, dd, J=4.67, 1.37 Hz), 8.35 (1H, dd, J=7.96, 1.37 Hz), 12.16 (1H, br s); ESIMS found for C₂₀H₁₉N₃O₂ m/z 334.1 (M+1).

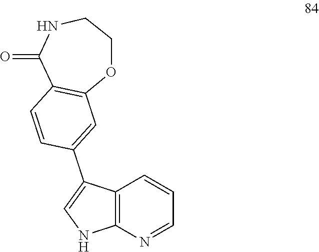 [SMILES: O=C1NCCOc2cc(-c3c[nH]c4ncccc34)ccc21]

### 8-(1H-Pyrrolo[2,3-b]pyridin-3-yl)-3,4-dihydrobenzo[f][1,4]oxazepin-5(2H)-one 84

White solid (19 mg, 0.068 mmol, 46.8% yield). ¹H NMR (499 MHz, DMSO-d₆) δ ppm 3.34-3.41 (2H, m), 4.31-4.37 (2H, m), 7.18 (1H, dd, J=7.96, 4.67 Hz), 7.35 (1H, d, J=1.65 Hz), 7.52 (1H, dd, J=8.23, 1.92 Hz), 7.86 (1H, d, J=8.23 Hz), 8.03 (1H, s), 8.26 (1H, br t, J=5.08 Hz), 8.29 (1H, dd, J=4.53, 1.51 Hz), 8.32 (1H, dd, J=8.10, 1.51 Hz), 12.05 (1H, br s); ESIMS found for C₁₆H₁₃N₃O₂ m/z 280.1 (M+1).

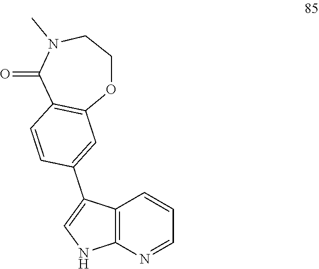 [SMILES: CN1CCOc2cc(-c3c[nH]c4ncccc34)ccc2C1=O]

### 4-Methyl-8-(1H-pyrrolo[2,3-b]pyridin-3-yl)-3,4-dihydrobenzo[f] [1,4]oxazepin-5(2H)-one 85

Beige solid (31 mg, 0.106 mmol, 75.1% yield). ¹H NMR (499 MHz, DMSO-d₆) δ ppm 3.10 (3H, s), 3.59 (2H, t, J=5.08 Hz), 4.41 (2H, t, J=4.94 Hz), 7.18 (1H, dd, J=7.96, 4.67 Hz), 7.35 (1H, d, J=1.65 Hz), 7.54 (1H, dd, J=7.96, 1.65 Hz), 7.72 (1H, d, J=8.23 Hz), 8.02 (1H, d, J=2.74 Hz), 8.29 (1H, dd, J=4.67, 1.37 Hz), 8.32 (1H, dd, J=7.96, 1.10 Hz), 12.04 (1H, br s); ESIMS found for C₁₇H₁₅N₃O₂ m/z 294.1 (M+1).

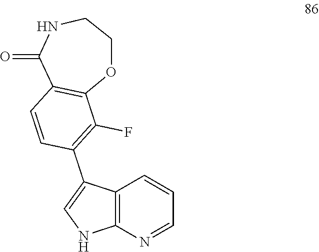 [SMILES: O=C1NCCOc2c1ccc(-c1c[nH]c3ncccc13)c2F]

### 9-Fluoro-8-(1H-pyrrolo[2,3-b]pyridin-3-yl)-3,4-dihydrobenzo[f] [1,4]oxazepin-5(2H)-one 86

White solid (18 mg, 0.061 mmol, 45.7% yield). ¹H NMR (499 MHz, DMSO-d₆) δ ppm 3.41 (2H, q, J=5.21 Hz), 4.39-4.45 (2H, m), 7.18 (1H, dd, J=8.10, 4.53 Hz), 7.47 (1H, dd, J=8.37, 6.72 Hz), 7.64 (1H, dd, J=8.23, 1.37 Hz), 7.84-7.91 (1H, m), 8.15 (1H, d, J=7.96 Hz), 8.31 (1H, dd, J=4.67, 1.37 Hz), 8.41 (1H, br t, J=5.21 Hz), 12.16 (1H, br s); ESIMS found for C₁₆H₁₂FN₃O₂ m/z 298.1 (M+1).

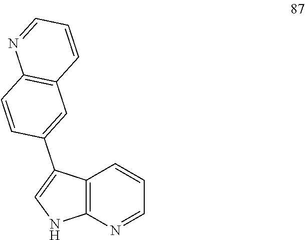 [SMILES: c1cnc2ccc(-c3c[nH]c4ncccc34)cc2c1]

### 6-(1H-Pyrrolo[2,3-b]pyridin-3-yl)quinoline 87

Light brown solid (10 mg, 0.041 mmol, 8.0% yield). ¹H NMR (499 MHz, DMSO-d₆) δ ppm 7.23 (1H, dd, J=7.96, 4.67 Hz), 7.53 (1H, dd, J=8.37, 4.25 Hz), 8.05 (1H, d, J=8.78 Hz), 8.11 (1H, s), 8.19 (1H, dd, J=8.78, 2.20 Hz), 8.30-8.34 (2H, m), 8.41-8.47 (1H, m), 8.53 (1H, dd, J=7.96, 1.37 Hz), 8.84 (1H, dd, J=4.12, 1.65 Hz), 12.06 (1H, br s); ESIMS found for C₁₆H₁₁N₃ m/z 246.1 (M+1).

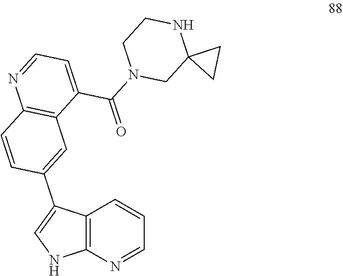 [SMILES: O=C(c1ccnc2ccc(-c3c[nH]c4ncccc34)cc12)N1CCNC2(CC2)C1]

### (6-(1H-Pyrrolo[2,3-b]pyridin-3-yl)quinolin-4-yl)(4,7-diazaspiro[2.5]octan-7-yl)methanone 88

Beige solid (20 mg, 0.052 mmol, 50.4% yield). ¹H NMR (499 MHz, DMSO-d₆) δ ppm 0.01-0.20 (1H, m), 0.32 (1H, br dd, J=6.86, 5.21 Hz), 0.53 (1H, br s), 0.68 (1H, br s), 2.59-2.70 (1H, m), 2.94 (1H, br s), 3.16 (1H, br s), 3.38-3.50 (1H, m), 3.56-3.92 (2H, m), 7.19-7.27 (1H, m), 7.35-7.45 (1H, m), 7.99-8.05 (1H, m), 8.07-8.16 (2H, m), 8.22 (1H, dd, J=8.78, 1.92 Hz), 8.27-8.33 (1H, m), 8.34 (1H, dd, J=4.67, 1.37 Hz), 8.83-8.93 (1H, m), 11.92 (1H, br s); ESIMS found for C₂₃H₂₁N₅O m/z 384.1 (M+1).

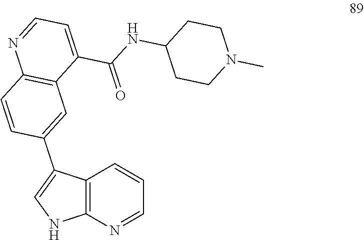 [SMILES: CN1CCC(NC(=O)c2ccnc3ccc(-c4c[nH]c5ncccc45)cc23)CC1]

### N-(1-Methylpiperidin-4-yl)-6-(1H-pyrrolo[2,3-b]pyridin-3-yl) quinoline-4-carboxamide 89

Yellow solid (20 mg, 0.052 mmol, 25.8% yield). ¹H NMR (499 MHz, DMSO-d₆) δ ppm 1.60 (2H, qd, J=11.76, 3.70 Hz), 1.85-1.95 (2H, m), 1.98-2.07 (2H, m), 2.17 (3H, s), 2.78 (2H, br d, J=11.53 Hz), 3.82-3.94 (1H, m), 7.23 (1H, dd, J=7.96, 4.67 Hz), 7.52 (1H, d, J=4.39 Hz), 8.09-8.13 (2H, m), 8.21 (1H, dd, J=8.78, 1.92 Hz), 8.30-8.35 (2H, m), 8.43 (1H, d, J=1.92 Hz), 8.78 (1H, d, J=7.68 Hz), 8.89 (1H, d, J=4.39 Hz), 12.12 (1H, s); ESIMS found for C₂₃H₂₃N₅O m/z 386.1 (M+1).

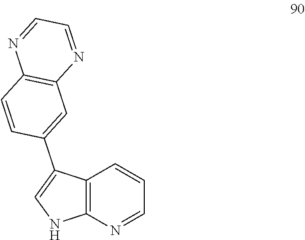 [SMILES: c1cnc2[nH]cc(-c3ccc4nccnc4c3)c2c1]

### 6-(1H-Pyrrolo[2,3-b]pyridin-3-yl)quinoxaline 90

Orange solid (25 mg, 0.102 mmol, 28.3% yield). ¹H NMR (499 MHz, DMSO-d₆) δ ppm 7.25 (1H, dd, J=7.96, 4.67 Hz), 8.13 (1H, d, J=8.78 Hz), 8.25 (1H, d, J=2.74 Hz), 8.33 (1H, br dd, J=13.04, 1.78 Hz), 8.33 (1H, d, J=1.92 Hz), 8.37 (1H, d, J=1.92 Hz), 8.48 (1H, dd, J=7.96, 0.82 Hz), 8.87 (1H, d, J=1.65 Hz), 8.94 (1H, d, J=1.92 Hz), 12.19 (1H, br s); ESIMS found for C₁₅H₁₀N₄ m/z 247.1 (M+1).

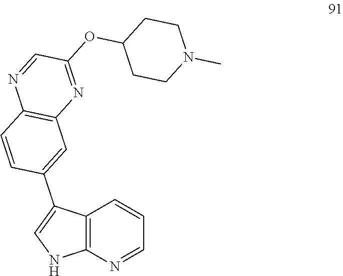 [SMILES: CN1CCC(Oc2cnc3ccc(-c4c[nH]c5ncccc45)cc3n2)CC1]

### 2-((1-Methylpiperidin-4-yl)oxy)-7-(1H-pyrrolo[2,3-b]pyridin-3-yl) quinoxaline 91

Pinkish solid (39.6 mg, 0.110 mmol, 35.5% yield). ¹H NMR (499 MHz, DMSO-d₆) δ ppm 1.75-1.85 (2H, m), 2.09 (2H, br dd, J=8.78, 3.84 Hz), 2.21 (3H, s), 2.23-2.29 (2H, m), 2.63-2.72 (2H, m), 5.22-5.32 (1H, m), 7.23 (1H, dd, J=7.96, 4.67 Hz), 8.00-8.03 (1H, m), 8.05-8.08 (2H, m), 8.19 (1H, d, J=1.37 Hz), 8.32 (1H, dd, J=4.67, 1.65 Hz), 8.43 (1H, dd, J=7.96, 1.37 Hz), 8.47 (1H, s), 12.14 (1H, br s); ESIMS found for C₂₁H₂₁N₅O m/z 360.1 (M+1).

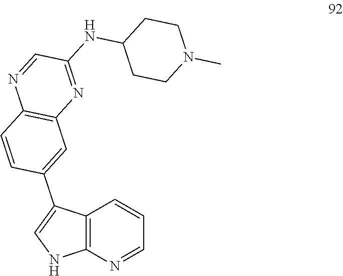 [SMILES: CN1CCC(Nc2cnc3ccc(-c4c[nH]c5ncccc45)cc3n2)CC1]

### N-(1-Methylpiperidin-4-yl)-7-(1H-pyrrolo[2,3-b]pyridin-3-yl) quinoxalin-2-amine 92

Brown solid (4.0 mg, 0.011 mmol, 2.7% yield). ¹H NMR (499 MHz, DMSO-d₆) δ ppm 1.45-1.61 (2H, m), 1.81-1.88 (2H, m), 2.03-2.11 (2H, m), 2.19 (3H, s), 2.76 (2H, br d, J=11.80 Hz), 3.86-3.97 (1H, m), 7.19-7.22 (1H, m), 7.49 (1H, br d, J=7.41 Hz), 8.07 (1H, s), 8.09-8.11 (1H, m), 8.21 (1H, s), 8.29-8.32 (2H, m), 8.37 (1H, dd, J=7.96, 1.37 Hz), 8.39 (1H, dd, J=8.10, 1.51 Hz), 12.03 (1H, br s); ESIMS found for C₂₁H₂₂N₆ m/z 359.1 (M+1).

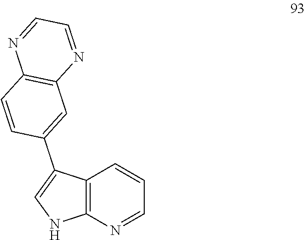 [SMILES: c1cnc2[nH]cc(-c3ccc4nccnc4c3)c2c1]

### 6-(1H-Pyrrolo[2,3-b]pyridin-3-yl)quinazoline 93

Dark yellow solid (20 mg, 0.081 mmol, 38.3% yield). ¹H NMR (500 MHz, DMSO-d₆) δ ppm 7.25 (1H, dd, J=7.96, 4.67 Hz), 8.05 (1H, d, J=8.78 Hz), 8.20 (1H, d, J=1.92 Hz), 8.34 (1H, dd, J=4.67, 1.65 Hz), 8.47 (1H, dd, J=8.92, 2.06 Hz), 8.51 (1H, d, J=1.92 Hz), 8.59 (1H, dd, J=7.96, 1.65 Hz), 9.24 (1H, s), 9.67 (1H, s), 12.15 (1H, br s); ESIMS found for C₁₅H₁₀N₄ m/z 247.05 (M+1).

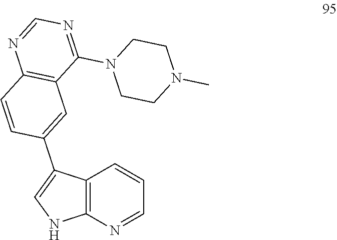 [SMILES: CN1CCN(c2ncnc3ccc(-c4c[nH]c5ncccc45)cc23)CC1]

### 4-(4-Methylpiperazin-1-yl)-6-(1H-pyrrolo[2,3-b]pyridin-3-yl) quinazoline 95

Yellow solid (37.0 mg, 0.107 mmol, 33.2% yield). ¹H NMR (499 MHz, DMSO-d₆) δ ppm 2.26 (3H, s), 2.53-2.58 (4H, m), 3.76-3.84 (4H, m), 7.24 (1H, dd, J=7.68, 4.67 Hz), 7.87 (1H, d, J=8.51 Hz), 8.08 (1H, d, J=2.19 Hz), 8.14 (1H, d, J=1.92 Hz), 8.21 (1H, dd, J=8.78, 1.92 Hz), 8.31 (1H, br d, J=11.53 Hz), 8.32 (1H, d, J=1.10 Hz), 8.61 (1H, s), 12.09 (1H, br s); ESIMS found for C₂₀H₂₀N₆ m/z 345.2 (M+1).

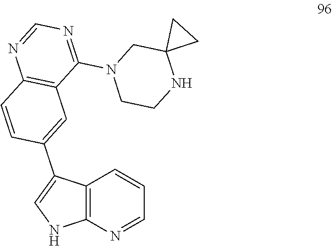 [SMILES: c1cnc2[nH]cc(-c3ccc4ncnc(N5CCNC6(CC6)C5)c4c3)c2c1]

### 6-(1H-Pyrrolo[2,3-b]pyridin-3-yl)-4-(4,7-diazaspiro[2.5]octan-7-yl) quinazoline 96

White solid (17 mg, 0.048 mmol, 55.8% yield). ¹H NMR (500 MHz, DMSO-d₆) δ ppm 0.44-0.55 (4H, m), 3.00 (2H, br d, J=3.84 Hz), 3.66 (2H, s), 3.75-3.83 (2H, m), 7.23 (1H, dd, J=7.96, 4.67 Hz), 7.85 (1H, d, J=8.51 Hz), 8.04 (1H, s), 8.10 (1H, d, J=1.92 Hz), 8.18 (1H, dd, J=8.78, 1.92 Hz), 8.27 (1H, dd, J=7.96, 1.65 Hz), 8.32 (1H, dd, J=4.67, 1.37 Hz), 8.58 (1H, s), 12.07 (1H, br s); ESIMS found for C₂₁H₂₀N₆ m/z 357.1 (M+1).

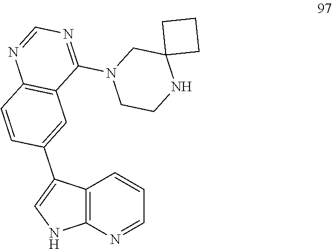 [SMILES: c1cnc2[nH]cc(-c3ccc4ncnc(N5CCNC6(CCC6)C5)c4c3)c2c1]

### 6-(1H-Pyrrolo[2,3-b]pyridin-3-yl)-4-(5,8-diazaspiro[3.5]nonan-8-yl) quinazoline 97

Beige solid (25 mg, 0.068 mmol, 12.5% yield). ¹H NMR (499 MHz, DMSO-d₆) δ ppm 1.60-1.69 (1H, m), 1.70-1.76 (1H, m), 1.79-1.90 (2H, m), 1.94-2.03 (2H, m), 2.90-2.97 (2H, m), 3.70 (4H, s), 7.22 (1H, dd, J=7.96, 4.67 Hz), 7.87 (1H, d, J=8.51 Hz), 8.04 (1H, d, J=2.47 Hz), 8.16 (1H, d, J=1.65 Hz), 8.20 (1H, dd, J=8.64, 1.78 Hz), 8.29 (1H, d, J=7.96 Hz), 8.32 (1H, dd, J=4.53, 1.24 Hz), 8.60 (1H, s), 12.09 (1H, br s); ESIMS found for C₂₂H₂₂N₆ m/z 371.1 (M+1).

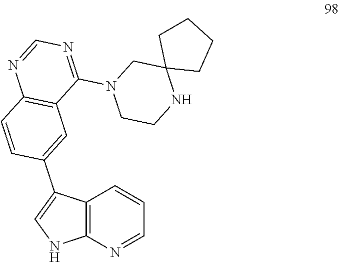 [SMILES: c1cnc2[nH]cc(-c3ccc4ncnc(N5CCNC6(CCCC6)C5)c4c3)c2c1]

### 9-(6-(1H-Pyrrolo[2,3-b]pyridin-3-yl)quinazolin-4-yl)-6,9-diazaspiro [4.5]decane 98

Beige solid (75 mg, 0.195 mmol, 33.9% yield). ¹H NMR (499 MHz, DMSO-d₆) δ ppm 1.43-1.50 (2H, m), 1.51-1.57 (2H, m), 1.57-1.66 (4H, m), 2.94-3.03 (2H, m), 3.58 (2H, s), 3.65-3.73 (2H, m), 7.22 (1H, dd, J=7.96, 4.67 Hz), 7.86 (1H, d, J=8.51 Hz), 8.03 (1H, d, J=1.92 Hz), 8.15 (1H, d, J=1.37 Hz), 8.18 (1H, dd, J=8.78, 1.92 Hz), 8.28 (1H, dd, J=7.96, 1.37 Hz), 8.32 (1H, dd, J=4.53, 1.24 Hz), 8.58 (1H, s), 12.08 (1H, br s); ESIMS found for C₂₃H₂₄N₆ m/z 385.1 (M+1).

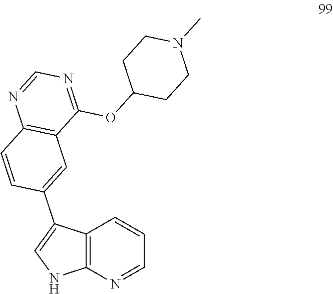 [SMILES: CN1CCC(Oc2ncnc3ccc(-c4c[nH]c5ncccc45)cc23)CC1]

### 4-((1-Methylpiperidin-4-yl)oxy)-6-(1H-pyrrolo[2,3-b]pyridin-3-yl) quinazoline 99

Off-white solid (30.7 mg, 0.085 mmol, 27.5% yield). ¹H NMR (499 MHz, DMSO-d₆) δ ppm 1.86-1.97 (2H, m), 2.02-2.12 (2H, m), 2.23 (3H, s), 2.29-2.40 (2H, m), 2.64 (2H, br dd, J=4.67, 2.47 Hz), 5.38-5.47 (1H, m), 7.24 (1H, dd, J=7.96, 4.67 Hz), 7.95 (1H, d, J=8.78 Hz), 8.15 (1H, d, J=2.47 Hz), 8.32-8.35 (2H, m), 8.35-8.37 (1H, m), 8.38 (1H, d, J=1.92 Hz), 8.74 (1H, s), 12.15 (1H, br s); ESIMS found for C₂₁H₂₁N₅O m/z 360.1 (M+1).

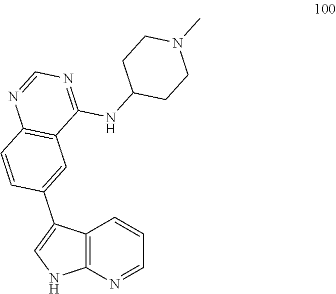 [SMILES: CN1CCC(Nc2ncnc3ccc(-c4c[nH]c5ncccc45)cc23)CC1]

### N-(1-Methylpiperidin-4-yl)-6-(1H-pyrrolo[2,3-b]pyridin-3-yl) quinazolin-4-amine 100

Grey solid (40.0 mg, 0.112 mmol, 27.2% yield). ¹H NMR (499 MHz, DMSO-d₆) δ ppm 1.70 (2H, qd, J=12.03, 3.70 Hz), 1.89-2.04 (4H, m), 2.20 (3H, s), 2.84 (2H, br d, J=11.53 Hz), 4.13-4.25 (1H, m), 7.22 (1H, dd, J=7.96, 4.67 Hz), 7.73 (1H, d, J=8.51 Hz), 7.95 (1H, d, J=7.68 Hz), 8.00 (1H, d, J=1.10 Hz), 8.14 (1H, dd, J=8.51, 1.92 Hz), 8.31 (1H, dd, J=4.53, 1.24 Hz), 8.40 (1H, dd, J=7.96, 1.10 Hz), 8.43 (1H, s), 8.53 (1H, d, J=1.65 Hz), 12.04 (1H, br s); ESIMS found for C₂₁H₂₂N₆ m/z 359.1 (M+1).

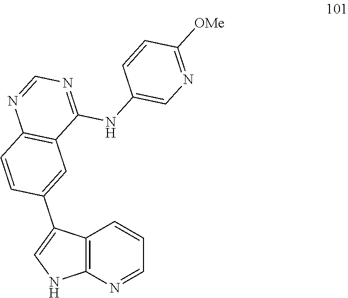 [SMILES: COc1ccc(Nc2ncnc3ccc(-c4c[nH]c5ncccc45)cc23)cn1]

### N-(6-Methoxypyridin-3-yl)-6-(1H-pyrrolo[2,3-b]pyridin-3-yl) quinazolin-4-amine 101

White solid (11 mg, 0.030 mmol, 25.3% yield). ¹H NMR (499 MHz, DMSO-d₆) δ ppm 3.88 (3H, s), 6.91 (1H, d, J=8.78 Hz), 7.23 (1H, dd, J=7.96, 4.67 Hz), 7.84 (1H, d, J=8.51 Hz), 8.05-8.10 (2H, m), 8.26 (1H, dd, J=8.64, 1.78 Hz), 8.33 (1H, dd, J=4.67, 1.37 Hz), 8.48-8.50 (2H, m), 8.52 (1H, dd, J=8.23, 1.37 Hz), 8.71 (1H, d, J=1.65 Hz), 9.89 (1H, s), 12.08 (1H, br s); ESIMS found for C₂₁H₁₆N₆O m/z 369.0 (M+1).

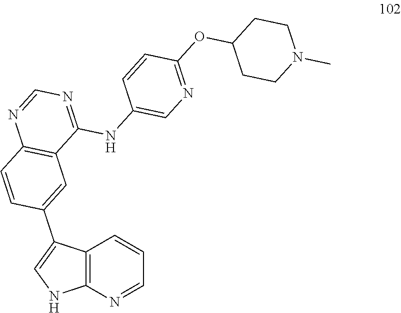 [SMILES: CN1CCC(Oc2ccc(Nc3ncnc4ccc(-c5c[nH]c6ncccc56)cc34)cn2)CC1]

### N-(6-((1-Methylpiperidin-4-yl)oxy)pyridin-3-yl)-6-(1H-pyrrolo[2,3-b] pyridin-3-yl)quinazolin-4-amine 102

White solid (48 mg, 0.106 mmol, 44.0% yield). ¹H NMR (499 MHz, DMSO-d₆) δ ppm 1.69 (2H, dtd, J=12.66, 9.25, 9.25, 3.70 Hz), 1.95-2.03 (2H, m), 2.13-2.20 (2H, m), 2.19 (3H, s), 2.58-2.70 (2H, m), 4.98 (1H, tt, J=8.68, 4.22 Hz), 6.86 (1H, d, J=8.78 Hz), 7.23 (1H, dd, J=7.96, 4.67 Hz), 7.84 (1H, d, J=8.78 Hz), 8.03-8.08 (2H, m), 8.26 (1H, dd, J=8.65, 1.78 Hz), 8.33 (1H, dd, J=4.67, 1.37 Hz), 8.46 (1H, d, J=2.74 Hz), 8.49 (1H, s), 8.52 (1H, dd, J=7.96, 1.10 Hz), 8.70 (1H, d, J=1.65 Hz), 9.87 (1H, s), 12.08 (1H, br s); ESIMS found for C₂₆H₂₅N₇O m/z 452.1 (M+1).

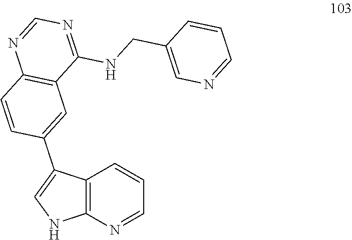 [SMILES: c1cncc(CNc2ncnc3ccc(-c4c[nH]c5ncccc45)cc23)c1]

### N-(Pyridin-3-ylmethyl)-6-(1H-pyrrolo[2,3-b]pyridin-3-yl)quinazolin-4-amine 103

Off-white solid (32 mg, 0.091 mmol, 28.6% yield). ¹H NMR (500 MHz, DMSO-d₆) δ ppm 4.85 (2H, br d, J=5.49 Hz), 7.22 (1H, br dd, J=7.82, 4.53 Hz), 7.35 (1H, br dd, J=7.68, 4.94 Hz), 7.76 (1H, br d, J=8.51 Hz), 7.80 (1H, br d, J=7.68 Hz), 8.01 (1H, br d, J=1.92 Hz), 8.19 (1H, br d, J=7.96 Hz), 8.31 (1H, br d, J=4.12 Hz), 8.44 (1H, s), 8.46 (1H, br d, J=4.12 Hz), 8.49 (1H, br d, J=7.68 Hz), 8.52 (1H, s), 8.65 (1H, br s), 8.93 (1H, br t, J=4.80 Hz), 12.04 (1H, br s); ESIMS found for C₂₁H₁₆N₆ m/z 353.0 (M+1).

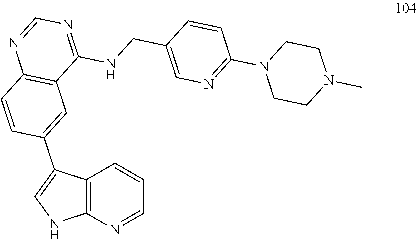 [SMILES: CN1CCN(c2ccc(CNc3ncnc4ccc(-c5c[nH]c6ncccc56)cc34)cn2)CC1]

### N-((6-(4-Methylpiperazin-1-yl)pyridin-3-yl)methyl)-6-(1H-pyrrolo[2,3-b]pyridin-3-yl)quinazolin-4-amine 104

Lime green solid (20 mg, 0.044 mmol, 22.9% yield). ¹H NMR (499 MHz, DMSO-d₆) δ ppm 2.18 (3H, s), 2.33-2.38 (4H, m), 3.39-3.46 (4H, m), 4.68 (2H, d, J=5.76 Hz), 6.79 (1H, d, J=8.78 Hz), 7.22 (1H, dd, J=7.96, 4.67 Hz), 7.59 (1H, dd, J=8.78, 2.47 Hz), 7.74 (1H, d, J=8.78 Hz), 7.99 (1H, s), 8.16 (1H, dd, J=8.78, 1.92 Hz), 8.19 (1H, d, J=2.47 Hz), 8.31 (1H, dd, J=4.53, 1.51 Hz), 8.45 (1H, s), 8.46 (1H, dd, J=7.96, 1.37 Hz), 8.48 (1H, d, J=1.65 Hz), 8.77 (1H, t, J=5.76 Hz), 12.03 (1H, br s); ESIMS found for C₂₆H₂₆N₈ m/z 451.1 (M+1).

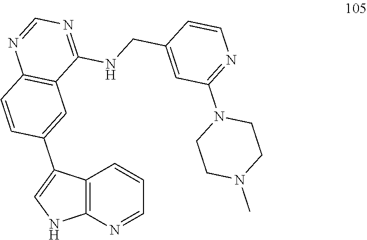 [SMILES: CN1CCN(c2cc(CNc3ncnc4ccc(-c5c[nH]c6ncccc56)cc34)ccn2)CC1]

### N-((2-(4-Methylpiperazin-1-yl)pyridin-4-yl)methyl)-6-(1H-pyrrolo [2,3-b]pyridin-3-yl)quinazolin-4-amine 105

White solid (21 mg, 0.047 mmol, 24.1% yield). ¹H NMR (499 MHz, DMSO-d₆) δ ppm 2.19 (3H, s), 2.36 (4H, t, J=4.94 Hz), 3.40-3.48 (4H, m), 4.75 (2H, d, J=5.76 Hz), 6.64 (1H, d, J=4.94 Hz), 6.83 (1H, s), 7.22 (1H, dd, J=7.96, 4.67 Hz), 7.76 (1H, d, J=8.51 Hz), 8.01 (1H, d, J=3.02 Hz), 8.02 (1H, s), 8.19 (1H, dd, J=8.65, 1.78 Hz), 8.32 (1H, dd, J=4.53, 1.51 Hz), 8.41 (1H, s), 8.50 (1H, dd, J=8.10, 1.24 Hz), 8.54 (1H, d, J=1.65 Hz), 8.87 (1H, t, J=5.90 Hz), 12.04 (1H, br s); ESIMS found for C₂₆H₂₆N₈ m/z 451.1 (M+1).

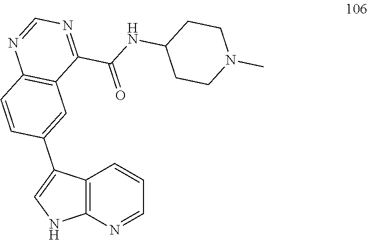 [SMILES: CN1CCC(NC(=O)c2ncnc3ccc(-c4c[nH]c5ncccc45)cc23)CC1]

### N-(1-Methylpiperidin-4-yl)-6-(1H-pyrrolo[2,3-b]pyridin-3-yl) quinazoline-4-carboxamide 106

Yellow solid (26 mg, 0.067 mmol, 29.4% yield). ¹H NMR (499 MHz, DMSO-d₆) δ ppm 1.61-1.76 (2H, m), 1.81-1.90 (2H, m), 1.98-2.09 (2H, m), 2.15-2.22 (3H, m), 2.78 (2H, br d, J=11.80 Hz), 3.84-3.98 (1H, m), 7.29 (1H, dd, J=7.96, 4.67 Hz), 8.12 (1H, d, J=8.78 Hz), 8.23 (1H, s), 8.35 (1H, dd, J=4.67, 1.37 Hz), 8.39 (1H, dd, J=7.96, 1.37 Hz), 8.51 (1H, dd, J=8.92, 2.06 Hz), 9.01 (1H, d, J=7.96 Hz), 9.11 (1H, d, J=1.92 Hz), 9.31 (1H, s), 12.22 (1H, br s); ESIMS found for C₂₂H₂₂N₆O m/z 387.1 (M+1).

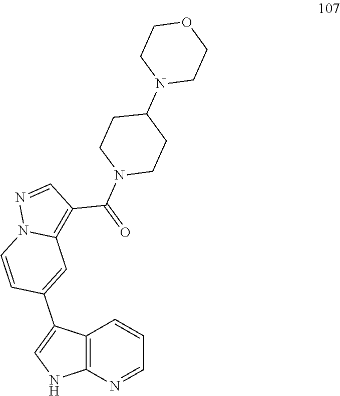 [SMILES: O=C(c1cnn2ccc(-c3c[nH]c4ncccc34)cc12)N1CCC(N2CCOCC2)CC1]

### (5-(1H-Pyrrolo[2,3-b]pyridin-3-yl)pyrazolo[1,5-a]pyridin-3-yl)(4-morpholinopiperidin-1-yl)methanone 107

Light brown solid (60 mg, 0.139 mmol, 33.6% yield). ¹H NMR (499 MHz, DMSO-d₆) δ ppm 1.43 (2H, qd, J=11.96, 3.83 Hz), 1.86 (2H, br d, J=11.50 Hz), 2.41-2.46 (1H, m), 2.46-2.49 (4H, m), 2.96-3.09 (2H, m), 3.52-3.61 (4H, m), 4.34 (2H, br d, J=12.59 Hz), 7.27 (1H, dd, J=7.80, 4.79 Hz), 7.48 (1H, dd, J=7.12, 1.92 Hz), 8.16 (1H, d, J=1.37 Hz), 8.21-8.24 (2H, m), 8.31-8.35 (2H, m), 8.77 (1H, d, J=7.39 Hz), 12.22 (1H, br s); ESIMS found for C₂₄H₂₆N₆O₂ m/z 431.2 (M+1).

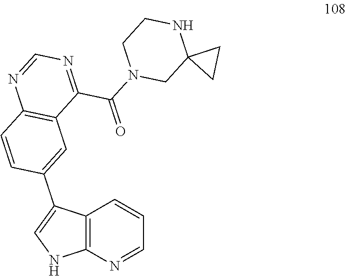 [SMILES: O=C(c1ncnc2ccc(-c3c[nH]c4ncccc34)cc12)N1CCNC2(CC2)C1]

### (6-(1H-Pyrrolo[2,3-b]pyridin-3-yl)quinazolin-4-yl)(4,7-diazaspiro [2.5]octan-7-yl)methanone 108

Beige solid (5 mg, 0.013 mmol, 48.5% yield). ¹H NMR (500 MHz, DMSO-d₆) δ ppm 0.12-0.25 (1H, m), 0.26-0.37 (1H, m), 0.49-0.57 (1H, m), 0.65-0.74(1H, m), 2.61 (1H, br d, J=4.67 Hz), 2.89-2.99 (1H, m), 3.09 (1H, s), 3.19 (1H, br d, J=3.02 Hz), 3.68 (1H, s), 3.77-3.86 (1H, m), 7.27 (1H, td, J=7.55, 4.67 Hz), 8.09-8.14 (1H, m), 8.14-8.19 (1H, m), 8.22 (1H, d, J=6.04 Hz), 8.30 (1H, td, J=7.89, 1.23 Hz), 8.36 (1H, dd, J=4.25, 2.33 Hz), 8.47-8.57 (1H, m), 9.21-9.30 (1H, m), 12.25 (1H, br s); ESIMS found for C₂₂H₂₀N₆O m/z 385.1 (M+1).

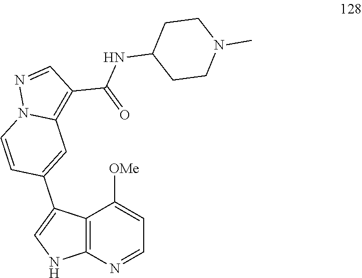 [SMILES: COc1ccnc2[nH]cc(-c3ccn4ncc(C(=O)NC5CCN(C)CC5)c4c3)c12]

### 5-(4-Methoxy-1H-pyrrolo[2,3-b]pyridin-3-yl)-N-(1-methylpiperidin-4-yl)pyrazolo[1,5-a]pyridine-3-carboxamide 128

Off-white solid (16 mg, 0.040 mmol, 14.7% yield). ¹H NMR (499 MHz, DMSO-d₆) δ ppm 1.57 (2H, qd, J=11.96, 3.83 Hz), 1.80 (2H, br dd, J=12.05, 1.92 Hz), 1.96 (2H, br t, J=10.81 Hz), 2.17 (3H, s), 2.78 (2H, br d, J=11.77 Hz), 3.70-3.82 (1H, m), 3.99 (3H, s), 6.77 (1H, d, J=5.75 Hz), 7.36 (1H, dd, J=7.39, 2.19 Hz), 7.82 (1H, d, J=2.74 Hz), 7.86 (1H, d, J=7.94 Hz), 8.19 (1H, d, J=5.48 Hz), 8.52 (1H, s), 8.65 (1H, dd, J=7.12, 0.82 Hz), 8.70 (1H, d, J=1.37 Hz), 12.06 (1H, br s); ESIMS found for C₂₂H₂₄N₆O₂ m/z 405.2 (M+1).

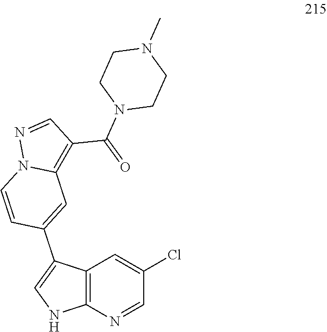 [SMILES: CN1CCN(C(=O)c2cnn3ccc(-c4c[nH]c5ncc(Cl)cc45)cc23)CC1]

### (5-(5-Chloro-1H-pyrrolo[2,3-b]pyridin-3-yl)pyrazolo[1,5-a]pyridin-3-yl)(4-methylpiperazin-1-yl)methanone 215

Light grey solid (12 mg, 0.030 mmol, 13.2% yield). ¹H NMR (499 MHz, DMSO-d₆) δ ppm 2.22 (3H, s), 2.39 (4H, t, J=5.08 Hz), 3.64-3.71 (4H, m), 7.48 (1H, dd, J=7.27, 2.06 Hz), 8.10 (1H, d, J=1.37 Hz), 8.25 (1H, s), 8.32 (1H, s), 8.35 (2H, q, J=2.47 Hz), 8.75-8.82 (1H, m), 12.48 (1H, br s); ESIMS found for C₂₀H₁₉ClN₆O m/z 395.0 (M+1).

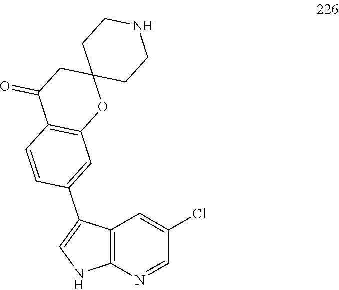 [SMILES: O=C1CC2(CCNCC2)Oc2cc(-c3c[nH]c4ncc(Cl)cc34)ccc21]

### 7-(5-Chloro-1H-pyrrolo[2,3-b]pyridin-3-yl)spiro[chromane-2,4′-piperidin]-4-one 226

White solid (28 mg, 0.076 mmol, 59.4% yield). ¹H NMR (499 MHz, DMSO-d₆) δ ppm 1.55-1.66 (2H, m), 1.84 (2H, br d, J=13.45 Hz), 2.69 (2H, dt, J=12.62, 3.98 Hz), 2.78 (2H, s), 2.85-2.94 (2H, m), 7.37 (1H, d, J=1.37 Hz), 7.45 (1H, dd, J=7.96, 1.65 Hz), 7.76 (1H, d, J=8.23 Hz), 8.23 (1H, s), 8.30 (1H, d, J=2.47 Hz), 8.39 (1H, d, J=2.20 Hz), 12.40 (1H, br s); ESIMS found for C₂₀H₁₈ClN₃O₂ m/z 368.1 (M+1).

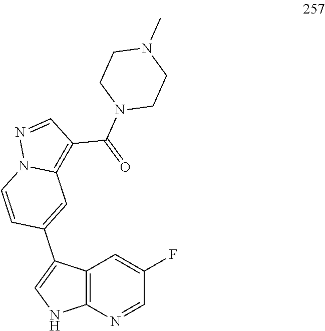 [SMILES: CN1CCN(C(=O)c2cnn3ccc(-c4c[nH]c5ncc(F)cc45)cc23)CC1]

### (5-(5-Fluoro-1H-pyrrolo[2,3-b]pyridin-3-yl)pyrazolo[1,5-a]pyridin-3-yl)(4-methylpiperazin-1-yl)methanone 257

Light yellow solid (20.0 mg, 0.053 mmol, 38.8% yield). ¹H NMR (499 MHz, DMSO-d₆) δ ppm 2.21 (3H, s), 2.38 (4H, t, J=4.94 Hz), 3.64-3.70 (4H, m), 7.51 (1H, dd, J=7.27, 2.06 Hz), 8.14 (1H, d, J=1.37 Hz), 8.26 (1H, s), 8.44 (1H, s), 8.61 (1H, d, J=1.65 Hz), 8.70 (1H, d, J=1.65 Hz), 8.82 (1H, d, J=7.41 Hz), 12.75 (1H, br s); ESIMS found for C₂₀H_{19N}FN₆O m/z 379.1 (M+1).

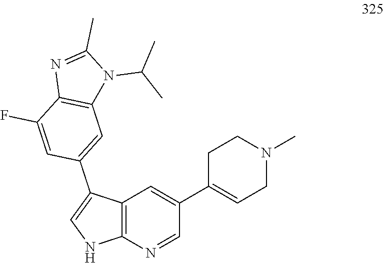 [SMILES: Cc1nc2c(F)cc(-c3c[nH]c4ncc(C5=CCN(C)CC5)cc34)cc2n1C(C)C]

### 4-Fluoro-1-isopropyl-2-methyl-6-(5-(1-methyl-1,2,3,6-tetrahydropyridin-4-yl)-1H-pyrrolo[2,3-b]pyridin-3-yl)-1H-benzo[d]imidazole 325

Yellow solid (15 mg, 0.037 mmol, 26.9% yield). ¹H NMR (499 MHz, DMSO-d₆) δ ppm 1.62 (6H, d, J=6.86 Hz), 2.29 (3H, s), 2.58-2.61 (4H, m), 2.59 (3H, s), 3.04 (2H, br d, J=2.74 Hz), 4.81 (1H, spt, J=6.90 Hz), 6.20 (1H, t, J=3.43 Hz), 7.29 (1H, dd, J=11.94, 0.96 Hz), 7.67 (1H, d, J=1.10 Hz), 7.91 (1H, d, J=2.47 Hz), 8.15 (1H, d, J=1.92 Hz), 8.43 (1H, d, J=1.92 Hz), 11.91 (1H, s); ESIMS found for C₂₄H₂₆FN₅ m/z 404.2 (M+1).

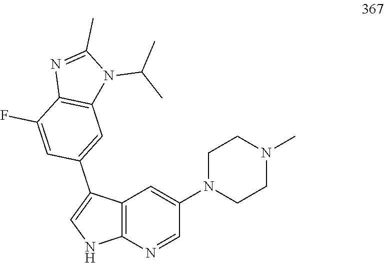 [SMILES: Cc1nc2c(F)cc(-c3c[nH]c4ncc(N5CCN(C)CC5)cc34)cc2n1C(C)C]

### 4-Fluoro-1-isopropyl-2-methyl-6-(5-(4-methylpiperazin-1-yl)-1H-pyrrolo[2,3-b]pyridin-3-yl)-1H-benzo[d]imidazole 367

Off-white solid (5 mg, 0.012 mmol, 22.4% yield). ¹H NMR (499 MHz, DMSO-d₆) δ ppm 1.61 (6H, d, J=6.86 Hz), 2.24 (3H, s), 2.51-2.53 (4H, m), 2.59 (3H, s), 3.11-3.16 (4H, m), 4.80 (1H, spt, J=6.80 Hz), 7.22-7.28 (1H, m), 7.63 (1H, d, J=0.82 Hz), 7.67 (1H, d, J=2.74 Hz), 7.82 (1H, s), 8.15 (1H, d, J=2.47 Hz), 11.67 (1H, br s); ESIMS found for C₂₃H₂₇FN₆ m/z 407.25 (M+1).

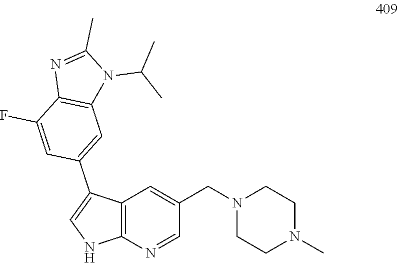 [SMILES: Cc1nc2c(F)cc(-c3c[nH]c4ncc(CN5CCN(C)CC5)cc34)cc2n1C(C)C]

### 4-Fluoro-1-isopropyl-2-methyl-6-(5-((4-methylpiperazin-1-yl)methyl)-1H-pyrrolo[2,3-b]pyridin-3-yl)-1H-benzo[d]imidazole 409

White solid (13 mg, 0.031 mmol, 35.5% yield). ¹H NMR (499 MHz, DMSO-d₆) δ ppm 1.63 (6H, d, J=7.14 Hz), 2.14 (3H, s), 2.20-2.35 (4H, m), 2.36-2.49 (4H, m), 2.59 (3H, s), 3.62 (2H, s), 4.81 (1H, spt, J=6.86 Hz), 7.28 (1H, dd, J=12.08, 1.10 Hz), 7.66 (1H, d, J=1.10 Hz), 7.93 (1H, d, J=2.47 Hz), 8.16 (1H, d, J=1.65 Hz), 8.19 (1H, d, J=1.65 Hz), 11.88 (1H, s); ESIMS found for C₂₄H₂₉FN₆ m/z 421.1 (M+1).

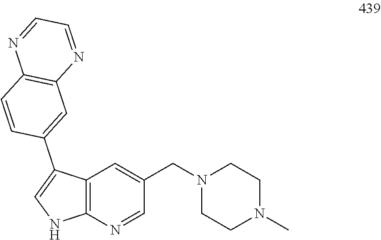 [SMILES: CN1CCN(Cc2cnc3[nH]cc(-c4ccc5nccnc5c4)c3c2)CC1]

### 6-(5-((4-Methylpiperazin-1-yl)methyl)-1H-pyrrolo[2,3-b]pyridin-3-yl) quinoxaline 439

Beige solid (6 mg, 0.017 mmol, 28.6% yield). ¹H NMR (499 MHz, DMSO-d₆) δ ppm 2.13 (3H, s), 2.19-2.36 (4H, m), 2.36-2.49 (4H, m), 3.64 (2H, s), 8.14 (1H, d, J=8.78 Hz), 8.23 (1H, s), 8.25 (1H, d, J=1.65 Hz), 8.31 (1H, dd, J=8.78, 1.92 Hz), 8.35 (2H, dd, J=3.29, 1.92 Hz), 8.88 (1H, d, J=1.92 Hz), 8.94 (1H, d, J=1.92 Hz), 12.16 (1H, s); ESIMS found for C₂₁H₂₂N₆ m/z 359.1 (M+1).

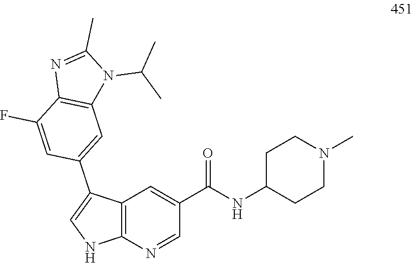 [SMILES: Cc1nc2c(F)cc(-c3c[nH]c4ncc(C(=O)NC5CCN(C)CC5)cc34)cc2n1C(C)C]

### 3-(4-Fluoro-1-isopropyl-2-methyl-1H-benzo[d]imidazol-6-yl)-N-(1-methylpiperidin-4-yl)-1H-pyrrolo[2,3-b]pyridine-5-carboxamide 451

White solid (5 mg, 0.011 mmol, 25.2% yield). ¹H NMR (499 MHz, DMSO-d₆) δ ppm 1.54-1.67 (2H, m), 1.63 (6H, d, J=6.86 Hz), 1.77-1.83 (2H, m), 1.92-1.99 (2H, m), 2.17 (3H, s), 2.60 (3H, s), 2.77 (2H, br d, J=11.53 Hz), 3.72-3.84 (1H, m), 4.82(1H, spt, J=6.86 Hz), 7.33 (1H, dd, J=11.94, 0.96 Hz), 7.72 (1H, d, J=1.10 Hz), 8.01 (1H, s), 8.38 (1H, d, J=7.68 Hz), 8.67 (1H, d, J=1.92 Hz), 8.76 (1H, d, J=1.92 Hz), 12.19 (1H, br s); ESIMS found for C₂₅H₂₉FN₆O m/z 449.2 (M+1).

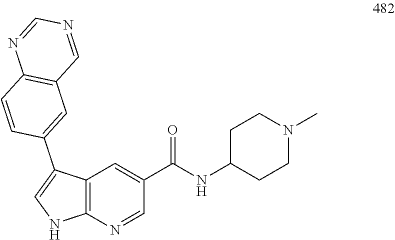 [SMILES: CN1CCC(NC(=O)c2cnc3[nH]cc(-c4ccc5ncncc5c4)c3c2)CC1]

### N-(1-Methylpiperidin-4-yl)-3-(quinazolin-6-yl)-1H-pyrrolo[2,3-b] pyridine-5-carboxamide 482

Yellowish white solid (4 mg, 0.010 mmol, 40.0% yield). ¹H NMR (499 MHz, DMSO-d₆) δ ppm 1.58-1.68 (2H, m), 1.80-1.87 (2H, m), 1.93-2.01 (2H, m), 2.17 (3H, s), 2.79 (2H, br d, J=11.80 Hz), 3.75-3.84 (1H, m), 8.10 (1H, d, J=8.78 Hz), 8.24 (1H, s), 8.40 (1H, d, J=7.68 Hz), 8.48 (1H, dd, J=8.64, 2.06 Hz), 8.51 (1H, d, J=1.65 Hz), 8.81 (1H, d, J=1.92 Hz), 8.87 (1H, d, J=2.20 Hz), 9.27 (1H, s), 9.68 (1H, s), 12.42 (1H, br s); ESIMS found for C₂₂H₂₂N₆O m/z 387.1 (M+1).

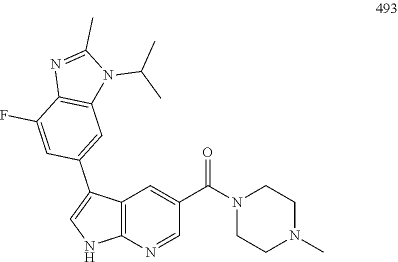 [SMILES: Cc1nc2c(F)cc(-c3c[nH]c4ncc(C(=O)N5CCN(C)CC5)cc34)cc2n1C(C)C]

### (3-(4-Fluoro-1-isopropyl-2-methyl-1H-benzo[d]imidazol-6-yl)-1H-pyrrolo[2,3-b]pyridin-5-yl)(4-methylpiperazin-1-yl)methanone 493

Pale yellow solid (3 mg, 0.007 mmol, 4.9% yield). ¹H NMR (499 MHz, DMSO-d₆) δ ppm 1.62 (6H, d, J=6.86 Hz), 2.20 (3H, s), 2.35 (4H, br s), 2.60 (3H, s), 3.48-3.65 (4H, m), 4.82 (1H, spt, J=6.93 Hz), 7.30 (1H, dd, J=11.94, 0.96 Hz), 7.68 (1H, d, J=1.10 Hz), 8.04 (1H, s), 8.24 (1H, d, J=1.92 Hz), 8.34 (1H, d, J=1.92 Hz), 12.19 (1H, s); ESIMS found for C₂₄H₂₇FN₆O m/z 435.2 (M+1).

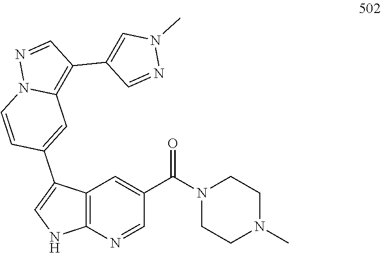 [SMILES: CN1CCN(C(=O)c2cnc3[nH]cc(-c4ccn5ncc(-c6cnn(C)c6)c5c4)c3c2)CC1]

### (3-(3-(1-Methyl-1H-pyrazol-4-yl)pyrazolo[1,5-a]pyridin-5-yl)-1H-pyrrolo[2,3-b]pyridin-5-yl)(4-methylpiperazin-1-yl)methanone 502

Light orange solid (10 mg, 0.023 mmol, 22.5% yield). ¹H NMR (499 MHz, DMSO-d₆) δ ppm 2.18 (3H, s), 2.27-2.39 (4H, m), 3.49-3.67 (4H, m), 3.91 (3H, s), 7.30 (1H, dd, J=7.14, 1.92 Hz), 7.90 (1H, s), 8.00 (1H, d, J=1.10 Hz), 8.18-8.21 (2H, m), 8.28 (1H, d, J=2.47 Hz), 8.35 (1H, d, J=1.65 Hz), 8.36-8.38 (1H, m), 8.67-8.72 (1H, m), 12.38 (1H, br s); ESIMS found for C₂₄H₂₄N₈O m/z 441.1 (M+1).

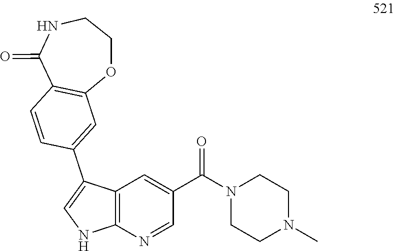 [SMILES: CN1CCN(C(=O)c2cnc3[nH]cc(-c4ccc5c(c4)OCCNC5=O)c3c2)CC1]

### 8-(5-(4-Methylpiperazine-1-carbonyl)-1H-pyrrolo[2,3-b]pyridin-3-yl)-3,4-dihydrobenzo[f][1,4]oxazepin-5(2H)-one 521

Light grey solid (6 mg, 0.015 mmol, 25.9% yield). ¹H NMR (499 MHz, DMSO-d₆) δ ppm 2.20 (3H, s), 2.27-2.42 (4H, m), 3.37 (2H, q, J=4.94 Hz), 3.42-3.71 (4H, m), 4.31-4.38 (2H, m), 7.35 (1H, d, J=1.65 Hz), 7.52 (1H, dd, J=8.23, 1.65 Hz), 7.87 (1H, d, J=8.23 Hz), 8.13 (1H, s), 8.28 (1H, br t, J=5.21 Hz), 8.30-8.32 (1H, m), 8.33 (1H, d, J=1.92 Hz), 12.31 (1H, br s); ESIMS found for C₂₂H₂₃N₅O₃ m/z 406.1 (M+1).

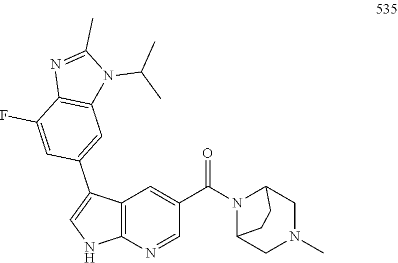 [SMILES: Cc1nc2c(F)cc(-c3c[nH]c4ncc(C(=O)N5C6CCC5CN(C)C6)cc34)cc2n1C(C)C]

### (3-(4-Fluoro-1-isopropyl-2-methyl-1H-benzo[d]imidazol-6-yl)-1H-pyrrolo[2,3-b]pyridin-5-yl)(3-methyl-3,8-diazabicyclo[3.2.1]octan-8-yl)methanone 535

Off-white solid (18.0 mg, 0.039 mmol, 27.5% yield). ¹H NMR (499 MHz, DMSO-d₆) δ ppm 1.61 (6H, d, J=6.86 Hz), 1.77-1.86 (4H, m), 2.18 (3H, s), 2.19-2.29 (2H, m), 2.63-2.75 (2H, m), 4.08-4.25 (1H, m), 4.54-4.69 (1H, m), 4.82 (1H, spt, J=6.93 Hz), 7.30 (1H, dd, J=11.94, 0.96 Hz), 7.69 (1H, d, J=1.37 Hz), 8.04 (1H, d, J=2.47 Hz), 8.30 (1H, d, J=1.92 Hz), 8.42 (1H, d, J=1.92 Hz), 12.22 (1H, br s); ESIMS found for C₂₆H₂₉FN₆O m/z 461.2 (M+1).

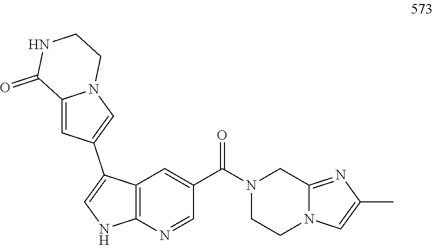 [SMILES: Cc1cn2c(n1)CN(C(=O)c1cnc3[nH]cc(-c4cc5n(c4)CCNC5=O)c3c1)CC2]

### 7-(5-(2-Methyl-5,6,7,8-tetrahydroimidazo[1,2-a]pyrazine-7-carbonyl)-1H-pyrrolo[2,3-b]pyridin-3-yl)-3,4-dihydropyrrolo[1,2-a]pyrazin-1(2H)-one 573

Beige solid (3 mg, 0.007 mmol, 22.8% yield). ¹H NMR (499 MHz, DMSO-d₆) δ ppm 2.05 (3H, br s), 3.24 (2H, br s), 3.52 (2H, br d, J=7.14 Hz), 3.99 (2H, br d, J=4.67 Hz), 4.09-4.15 (2H, m), 4.73 (2H, br s), 6.81 (1H, s), 7.03 (1H, d, J=1.10 Hz), 7.52 (1H, s), 7.66 (1H, br s), 7.82 (1H, s), 8.32 (1H, s), 8.35 (1H, s), 11.93 (1H, br s); ESIMS found for C₂₂H₂₁N₇O₂ m/z 416.1 (M+1).

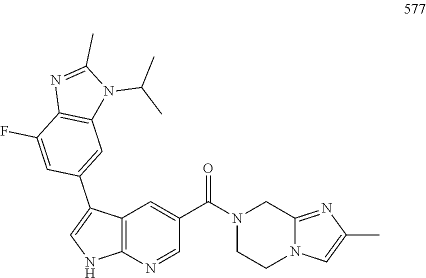 [SMILES: Cc1cn2c(n1)CN(C(=O)c1cnc3[nH]cc(-c4cc(F)c5nc(C)n(C(C)C)c5c4)c3c1)CC2]

### (3-(4-Fluoro-1-isopropyl-2-methyl-1H-benzo[d]imidazol-6-yl)-1H-pyrrolo[2,3-b]pyridin-5-yl)(2-methyl-5,6-dihydroimidazo[1,2-a]pyrazin-7(8H)-yl)methanone 577

Beige solid (10 mg, 0.021 mmol, 14.9% yield). ¹H NMR (499 MHz, DMSO-d₆) δ ppm 1.60 (6H, d, J=6.86 Hz), 2.05 (3H, s), 2.59 (3H, s), 3.95 (2H, br s), 4.00 (2H, br d, J=4.67 Hz), 4.73 (2H, s), 4.81 (1H, dt, J=13.79, 6.96 Hz), 6.80 (1H, s), 7.31 (1H, d, J=12.08 Hz), 7.68 (1H, s), 8.07 (1H, s), 8.36 (1H, d, J=1.37 Hz), 8.42 (1H, d, J=1.65 Hz), 12.17 (1H, br s); ESIMS found for C₂₆H₂₆FN₇O m/z 472.1 (M+1).

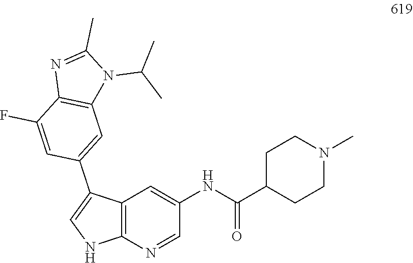 [SMILES: Cc1nc2c(F)cc(-c3c[nH]c4ncc(NC(=O)C5CCN(C)CC5)cc34)cc2n1C(C)C]

### N-(3-(4-Fluoro-1-isopropyl-2-methyl-1H-benzo[d]imidazol-6-yl)-1H-pyrrolo[2,3-b]pyridin-5-yl)-1-methylpiperidine-4-carboxamide 619

Yellow solid (30 mg, 0.067 mmol, 60.6% yield). ¹H NMR (499 MHz, DMSO-d₆) δ ppm 1.62 (6H, d, J=6.86 Hz), 1.65-1.74 (2H, m), 1.75-1.83 (2H, m), 1.88 (2H, td, J=11.60, 2.06 Hz), 2.16 (3H, s), 2.31 (1H, tt, J=11.46, 3.91 Hz), 2.59 (3H, s), 2.82 (2H, br d, J=11.53 Hz), 4.79 (1H, spt, J=6.93 Hz), 7.24 (1H, dd, J=12.08, 0.82 Hz), 7.65 (1H, d, J=1.10 Hz), 7.92 (1H, s), 8.40 (1H, d, J=2.20 Hz), 8.66 (1H, d, J=2.20 Hz), 9.95 (1H, s), 11.84 (1H, s); ESIMS found for C₂₅H₂₉FN₆O m/z 449.2 (M+1).

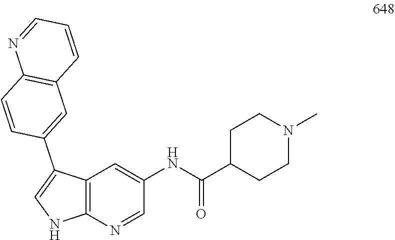 [SMILES: CN1CCC(C(=O)Nc2cnc3[nH]cc(-c4ccc5ncccc5c4)c3c2)CC1]

### 1-Methyl-N-(3-(quinolin-6-yl)-1H-pyrrolo[2,3-b]pyridin-5-yl) piperidine-4-carboxamide 648

Yellow solid (5 mg, 0.013 mmol, 15.2% yield). ¹H NMR (499 MHz, DMSO-d₆) δ ppm 1.65-1.77 (2H, m), 1.79-1.85 (2H, m), 1.97 (2H, br t, J=11.11 Hz), 2.22 (3H, s), 2.29-2.39 (1H, m), 2.89 (2H, br d, J=11.53 Hz), 7.54 (1H, dd, J=8.37, 4.25 Hz), 8.04 (1H, d, J=2.74 Hz), 8.06-8.10 (1H, m), 8.11-8.14 (1H, m), 8.19 (1H, s), 8.38 (1H, dd, J=8.37, 1.24 Hz), 8.48 (1H, d, J=2.20 Hz), 8.71 (1H, d, J=2.20 Hz), 8.85 (1H, dd, J=4.12, 1.65 Hz), 9.99 (1H, s), 11.99 (1H, s); ESIMS found for C₂₃H₂₃N₅O m/z 386.15 (M+1).

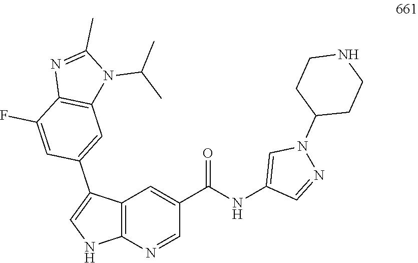 [SMILES: Cc1nc2c(F)cc(-c3c[nH]c4ncc(C(=O)Nc5cnn(C6CCNCC6)c5)cc34)cc2n1C(C)C]

### 3-(4-Fluoro-1-isopropyl-2-methyl-1H-benzo[d]imidazol-6-yl)-N-(1-(piperidin-4-yl)-1H-pyrazol-4-yl)-1H-pyrrolo[2,3-b]pyridine-5-carboxamide 661

Beige solid (48 mg, 0.096 mmol, 96.0% yield). ¹H NMR (499 MHz, DMSO-d₆) δ ppm 1.63 (6H, d, J=7.14 Hz), 1.80-1.90 (2H, m), 2.01 (2H, br d, J=10.15 Hz), 2.61 (3H, s), 2.68-2.79 (2H, m), 3.15 (2H, br d, J=12.90 Hz), 4.28 (1H, tt, J=11.39, 4.25 Hz), 4.77-4.90 (1H, m), 7.37(1H, d, J=11.53 Hz), 7.61 (1H, s), 7.73 (1H, d, J=0.82 Hz), 8.06 (1H, s), 8.08 (1H, s), 8.79 (1H, d, J=2.20 Hz), 8.87 (1H, d, J=1.92 Hz), 10.55 (1H, s), 12.26 (1H, br s); ESIMS found for C₂₇H₂₉FN₈O m/z 501.2 (M+1).

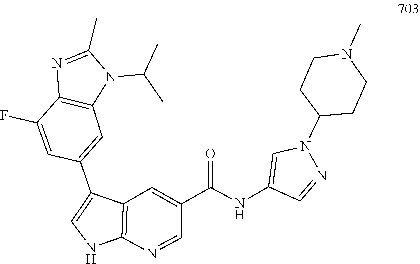 [SMILES: Cc1nc2c(F)cc(-c3c[nH]c4ncc(C(=O)Nc5cnn(C6CCN(C)CC6)c5)cc34)cc2n1C(C)C]

### 3-(4-Fluoro-1-isopropyl-2-methyl-1H-benzo[d]imidazol-6-yl)-N-(1-(1-methylpiperidin-4-yl)-1H-pyrazol-4-yl)-1H-pyrrolo[2,3-b]pyridine-5-carboxamide 703

Off-white solid (5 mg, 0.010 mmol, 10.8% yield). ¹H NMR (499 MHz, DMSO-d₆) δ ppm 1.63 (6H, d, J=6.86 Hz), 1.91-1.98 (4H, m), 2.01-2.08 (2H, m), 2.20 (3H, s), 2.61 (3H, s), 2.85 (2H, br d, J=11.25 Hz), 4.11 (1H, tt, J=10.33, 5.32 Hz), 4.83 (1H, quin, J=6.93 Hz), 7.37 (1H, d, J=12.08 Hz), 7.62 (1H, s), 7.73 (1H, d, J=0.82 Hz), 8.06 (1H, s), 8.07 (1H, s), 8.79 (1H, d, J=2.20 Hz), 8.87 (1H, d, J=1.92 Hz), 10.54 (1H, s), 12.27 (1H, br s); ESIMS found for C₂₈H₃₁FN₈O m/z 515.3 (M+1).

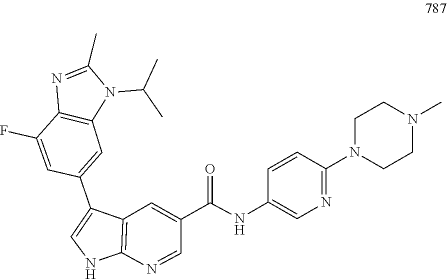 [SMILES: Cc1nc2c(F)cc(-c3c[nH]c4ncc(C(=O)Nc5ccc(N6CCN(C)CC6)nc5)cc34)cc2n1C(C)C]

### 3-(4-Fluoro-1-isopropyl-2-methyl-1H-benzo[d]imidazol-6-yl)-N-(6-(4-methylpiperazin-1-yl)pyridin-3-yl)-1H-pyrrolo[2,3-b]pyridine-5-carboxamide 787

Brown solid (25 mg, 0.048 mmol, 12.3% yield). ¹H NMR (499 MHz, DMSO-d₆) δ ppm 1.63 (6H, d, J=7.14 Hz), 2.23 (3H, s), 2.43 (4H, br s), 2.60 (3H, s), 3.45 (4H, br s), 4.82 (1H, spt, J=7.00 Hz), 6.87 (1H, d, J=9.06 Hz), 7.38 (1H, dd, J=11.94, 0.96 Hz), 7.75 (1H, d, J=1.10 Hz), 7.93 (1H, dd, J=9.19, 2.61 Hz), 8.06 (1H, d, J=2.74 Hz), 8.46 (1H, d, J=2.47 Hz), 8.81 (1H, d, J=1.92 Hz), 8.88 (1H, d, J=1.92 Hz), 10.27 (1H, s), 12.27 (1H, s); ESIMS found for C₂₉H₃₁FN₈O m/z 527.3 (M+1).

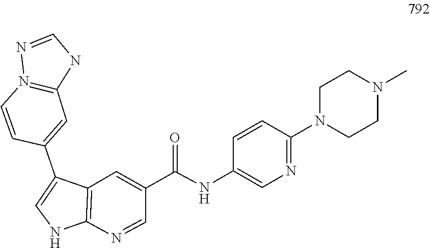

### 3-([1,2,4]Triazolo[1,5-a]pyridin-7-yl)-N-(6-(4-methylpiperazin-1-yl) pyridin-3-yl)-1H-pyrrolo[2,3-b]pyridine-5-carboxamide 792

Beige solid (5 mg, 0.011 mmol, 14.9% yield). ¹H NMR (499 MHz, DMSO-d₆) δ ppm 2.19-2.26 (3H, m), 2.37-2.44 (4H, m), 3.42-3.50 (4H, m), 6.89 (1H, d, J=9.03 Hz), 7.70 (1H, dd, J=7.26, 1.78 Hz), 7.92 (1H, dd, J=9.17, 2.60 Hz), 8.28 (1H, d, J=1.09 Hz), 8.41 (1H, s), 8.47 (1H, d, J=2.74 Hz), 8.49 (1H, s), 8.92 (1H, d, J=1.92 Hz), 8.98-9.01 (1H, m), 9.02 (1H, d, J=1.92 Hz), 10.36 (1H, s), 12.58 (1H, br s); ESIMS found for C₂₄H₂₃N₉O m/z 454.1 (M+1).

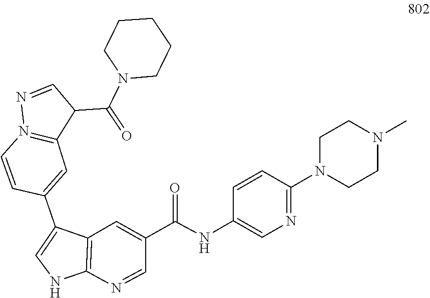

### N-(6-(4-Methylpiperazin-1-yl)pyridin-3-yl)-3-(3-(piperidine-1-carbonyl)pyrazolo[1,5-a]pyridin-5-yl)-1H-pyrrolo[2,3-b]pyridine-5-carboxamide 802

Beige solid (6 mg, 0.011 mmol, 17.8% yield). ¹H NMR (499 MHz, DMSO-d₆) δ ppm 1.51-1.65 (6H, m), 2.22 (3H, s), 2.38-2.46 (4H, m), 3.41-3.49 (4H, m), 3.60-3.69 (4H, m), 6.88 (1H, d, J=9.06 Hz), 7.52 (1H, dd, J=7.41, 1.92 Hz), 7.93 (1H, dd, J=9.06, 2.74 Hz), 8.15 (1H, d, J=1.37 Hz), 8.21 (1H, s), 8.33 (1H, s), 8.48 (1H, d, J=2.74 Hz), 8.81-8.85 (1H, m), 8.88 (1H, d, J=1.92 Hz), 8.92 (1H, d, J=1.92 Hz), 10.31 (1H, s), 12.53 (1H, br s); ESIMS found for C₃₁H₃₃N₉O₂ m/z 564.2 (M+1).

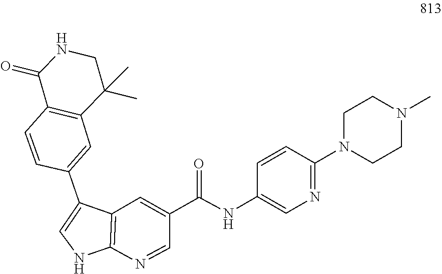 [SMILES: CN1CCN(c2ccc(NC(=O)c3cnc4[nH]cc(-c5ccc6c(c5)C(C)(C)CNC6=O)c4c3)cn2)CC1]

### 3-(4,4-Dimethyl-1-oxo-1,2,3,4-tetrahydroisoquinolin-6-yl)-N-(6-(4-methylpiperazin-1-yl)pyridin-3-yl)-1H-pyrrolo[2,3-b]pyridine-5-carboxamide 813

Beige solid (5 mg, 0.010 mmol, 13.6% yield). ¹H NMR (499 MHz, DMSO-d₆) δ ppm 1.37 (6H, s), 2.22 (3H, s), 2.37-2.45 (4H, m), 3.21 (2H, d, J=2.74 Hz), 3.41-3.50 (4H, m), 6.87 (1H, d, J=9.31 Hz), 7.74 (1H, d, J=1.37 Hz), 7.78 (1H, dd, J=8.08, 1.78 Hz), 7.91-7.94 (2H, m), 7.96 (1H, d, J=8.21 Hz), 8.16 (1H, s), 8.47 (1H, d, J=2.74 Hz), 8.83 (1H, d, J=2.19 Hz), 8.88 (1H, d, J=1.92 Hz), 10.27 (1H, s), 12.39 (1H, br s); ESIMS found for C₂₉H₃₁N₇O₂ m/z 510.2 (M+1).

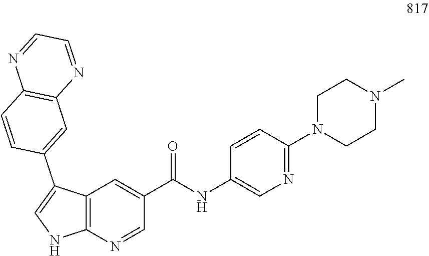 [SMILES: CN1CCN(c2ccc(NC(=O)c3cnc4[nH]cc(-c5ccc6nccnc6c5)c4c3)cn2)CC1]

### N-(6-(4-Methylpiperazin-1-yl)pyridin-3-yl)-3-(quinoxalin-6-yl)-1H-pyrrolo[2,3-b]pyridine-5-carboxamide 817

Beige solid (32 mg, 0.069 mmol, 40.3% yield). ¹H NMR (499 MHz, DMSO-d₆) δ ppm 2.22 (3H, s), 2.41 (4H, t, J=4.94 Hz), 3.42-3.50 (4H, m), 6.89 (1H, d, J=9.06 Hz), 7.92 (1H, dd, J=9.06, 2.74 Hz), 8.18 (1H, d, J=8.78 Hz), 8.35-8.40 (2H, m), 8.49 (1H, d, J=2.47 Hz), 8.50 (1H, d, J=1.92 Hz), 8.90 (1H, d, J=1.92 Hz), 8.93 (1H, d, J=1.92 Hz), 8.96 (1H, d, J=1.65 Hz), 9.02 (1H, d, J=1.65 Hz), 10.35 (1H, s), 12.53 (1H, br s); ESIMS found for C₂₆H₂₄N₈O m/z 465.1 (M+1).

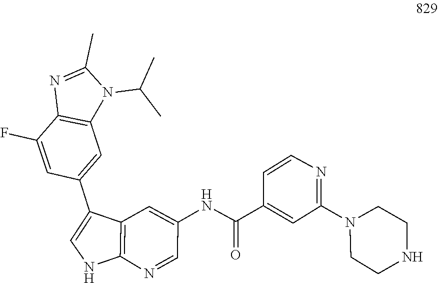 [SMILES: Cc1nc2c(F)cc(-c3c[nH]c4ncc(NC(=O)c5ccnc(N6CCNCC6)c5)cc34)cc2n1C(C)C]

### N-(3-(4-Fluoro-1-isopropyl-2-methyl-1H-benzo[d]imidazol-6-yl)-1H-pyrrolo[2,3-b]pyridin-5-yl)-2-(piperazin-1-yl)isonicotinamide 829

Off-white solid (12 mg, 0.023 mmol, 71.7% yield). ¹H NMR (499 MHz, DMSO-d₆) δ ppm 1.63 (6H, d, J=6.86 Hz), 2.59 (3H, s), 2.80-2.87 (4H, m), 3.50-3.56 (4H, m), 4.80 (1H, spt, J=6.86 Hz), 7.10 (1H, d, J=5.21 Hz), 7.26-7.30 (2H, m), 7.69(1H, d, J=1.10 Hz), 7.98 (1H, d, J=2.47 Hz), 8.27 (1H, d, J=4.94 Hz), 8.58 (1H, d, J=2.20 Hz), 8.75 (1H, d, J=2.20 Hz), 10.45 (1H, s), 11.94 (1H, s); ESIMS found for C₂₈H₂₉FN₈O m/z 513.3 (M+1).

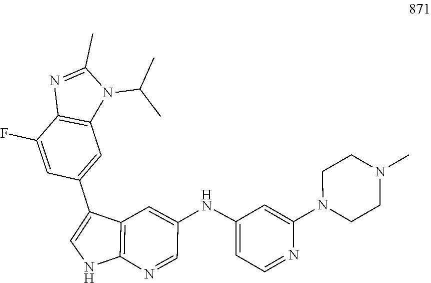 [SMILES: Cc1nc2c(F)cc(-c3c[nH]c4ncc(Nc5ccnc(N6CCN(C)CC6)c5)cc34)cc2n1C(C)C]

### 3-(4-Fluoro-1-isopropyl-2-methyl-1H-benzo[d]imidazol-6-yl)-N-(2-(4-methylpiperazin-1-yl)pyridin-4-yl)-1H-pyrrolo[2,3-b]pyridin-5-amine 871

Off-white solid (5 mg, 0.010 mmol, 10.7% yield). ¹H NMR (499 MHz, DMSO-d₆) δ ppm 1.58 (6H, d, J=6.86 Hz), 2.18 (3H, s), 2.31-2.38 (4H, m), 2.58 (3H, s), 3.33-3.37 (4H, m), 4.79 (1H, spt, J=6.93 Hz), 6.17 (1H, d, J=1.65 Hz), 6.27 (1H, dd, J=5.76, 1.92 Hz), 7.25 (1H, dd, J=12.08, 1.10 Hz), 7.63 (1H, d, J=1.10 Hz), 7.78 (1H, d, J=5.49 Hz), 7.95 (1H, s), 8.01 (1H, d, J=2.47 Hz), 8.15 (1H, d, J=2.47 Hz), 8.43 (1H, s), 11.92 (1H, br s); ESIMS found for C₂₈H₃₁FN₈ m/z 499.3 (M+1).

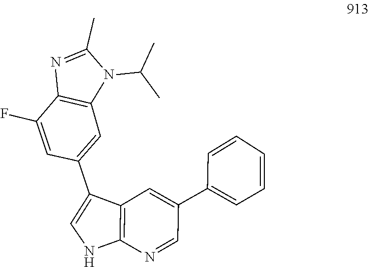 [SMILES: Cc1nc2c(F)cc(-c3c[nH]c4ncc(-c5ccccc5)cc34)cc2n1C(C)C]

### 4-Fluoro-1-isopropyl-2-methyl-6-(5-phenyl-1H-pyrrolo[2,3-b]pyridin-3-yl)-1H-benzo[d]imidazole 913

White solid (22 mg, 0.057 mmol, 43.5% yield ¹H NMR (499 MHz, DMSO-d₆) δ ppm 1.62 (6H, d, J=6.86 Hz), 2.60 (3H, s), 4.82 (1H, spt, J=6.95 Hz), 7.35-7.40 (2H, m), 7.50 (2H, t, J=7.68 Hz), 7.74 (1H, d, J=1.37 Hz), 7.76 (1H, d, J=1.10 Hz), 7.77-7.78 (1H, m), 7.99 (1H, d, J=1.37 Hz), 8.40 (1H, d, J=1.92 Hz), 8.58 (1H, d, J=1.92 Hz), 12.03 (1H, br s); ESIMS found for C₂₄H₂₁FN₄ m/z 385.2 (M+1).

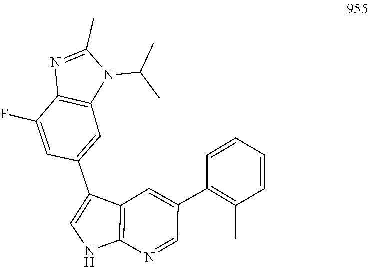 [SMILES: Cc1ccccc1-c1cnc2[nH]cc(-c3cc(F)c4nc(C)n(C(C)C)c4c3)c2c1]

### 4-Fluoro-1-isopropyl-2-methyl-6-(5-(o-tolyl)-1H-pyrrolo[2,3-b] pyridin-3-yl)-1H-benzo[d]imidazole 955

White solid (47 mg, 0.118 mmol, 63.5% yield). ¹H NMR (499 MHz, DMSO-d₆) δ ppm 1.59 (6H, d, J=7.14 Hz), 2.31 (3H, s), 2.58 (3H, s), 4.79 (1H, spt, J=6.82 Hz), 7.28-7.33 (3H, m), 7.33-7.37 (2H, m), 7.68 (1H, d, J=1.10 Hz), 8.00 (1H, d, J=2.74 Hz), 8.14 (1H, d, J=1.92 Hz), 8.26 (1H, d, J=1.92 Hz), 12.03 (1H, br s); ESIMS found for C₂₅H₂₃FN₄ m/z 399.2 (M+1).

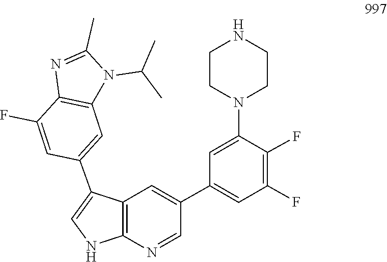 [SMILES: Cc1nc2c(F)cc(-c3c[nH]c4ncc(-c5cc(F)c(F)c(N6CCNCC6)c5)cc34)cc2n1C(C)C]

### 6-(5-(3,4-Difluoro-5-(piperazin-1-yl)phenyl)-1H-pyrrolo[2,3-b] pyridin-3-yl)-4-fluoro-1-isopropyl-2-methyl-1H-benzo[d]imidazole 997

White solid (29 mg, 0.058 mmol, 69.5% yield). ¹H NMR (499 MHz, DMSO-d₆) δ ppm 1.63 (6H, d, J=6.86 Hz), 2.60 (3H, s), 2.85-2.91 (4H, m), 3.03-3.10 (4H, m), 4.83 (1H, spt, J=6.86 Hz), 7.15 (1H, br d, J=7.14 Hz), 7.36 (1H, dd, J=12.08, 0.82 Hz), 7.42 (1H, ddd, J=11.25, 6.59, 1.92 Hz), 7.78 (1H, d, J=1.10 Hz), 7.99 (1H, d, J=2.47 Hz), 8.40 (1H, d, J=2.20 Hz), 8.59 (1H, d, J=2.20 Hz), 12.06 (1H, br s); ESIMS found for C₂₈H₂₇F₃N₆ m/z 505.3 (M+1).

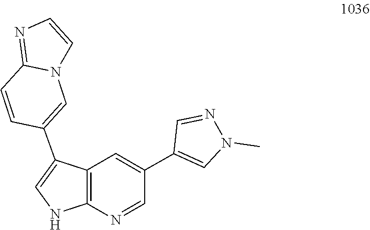 [SMILES: Cn1cc(-c2cnc3[nH]cc(-c4ccc5nccn5c4)c3c2)cn1]

### 6-(5-(1-Methyl-1H-pyrazol-4-yl)-1H-pyrrolo[2,3-b]pyridin-3-yl) imidazo[1,2-a]pyridine 1036

Tan solid (4 mg, 0.013 mmol, 19.9% yield). ¹H NMR (499 MHz, DMSO-d₆) δ ppm 3.90 (3H, s), 7.58 (1H, d, J=0.82 Hz), 7.61-7.65 (1H, m), 7.65-7.69 (1H, m), 7.95 (1H, d, J=2.20 Hz), 8.02 (2H, d, J=0.82 Hz), 8.24 (1H, s), 8.46 (1H, d, J=1.92 Hz), 8.56 (1H, d, J=1.92 Hz), 8.97 (1H, s), 11.96 (1H, br s); ESIMS found for C₁₈H₁₄N₆ m/z 315.05 (M+1).

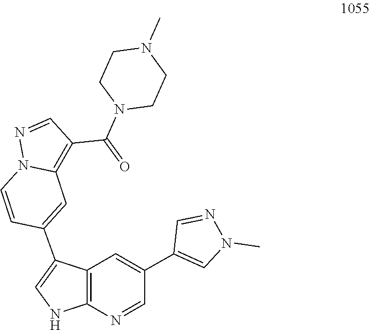 [SMILES: CN1CCN(C(=O)c2cnn3ccc(-c4c[nH]c5ncc(-c6cnn(C)c6)cc45)cc23)CC1]

### (5-(5-(1-Methyl-1H-pyrazol-4-yl)-1H-pyrrolo[2,3-b]pyridin-3-yl) pyrazolo[1,5-a]pyridin-3-yl)(4-methylpiperazin-1-yl)methanone 1055

Light yellow solid (5 mg, 0.011 mmol, 29.3% yield). ¹H NMR (499 MHz, DMSO-d₆) δ ppm 2.17 (3H, s), 2.34-2.40 (4H, m), 3.65-3.70 (4H, m), 3.90 (3H, s), 7.51 (1H, dd, J=7.27, 2.06 Hz), 7.93 (1H, d, J=0.82 Hz), 8.13 (1H, d, J=1.10 Hz), 8.18 (1H, d, J=2.47 Hz), 8.21 (1H, s), 8.24 (1H, s), 8.42 (1H, d, J=1.92 Hz), 8.56 (1H, d, J=1.92 Hz), 8.78(1H, d, J=7.41 Hz), 12.19 (1H, br s); ESIMS found for C₂₄H₂₄N₈O m/z 441.1 (M+1).

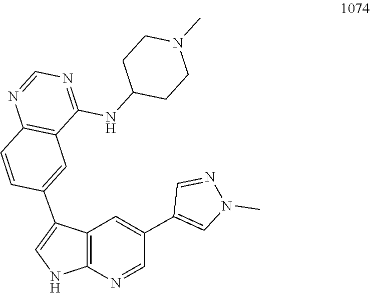 [SMILES: CN1CCC(Nc2ncnc3ccc(-c4c[nH]c5ncc(-c6cnn(C)c6)cc45)cc23)CC1]

### 6-(5-(1-Methyl-1H-pyrazol-4-yl)-1H-pyrrolo[2,3-b]pyridin-3-yl)-N-(1-methylpiperidin-4-yl)quinazolin-4-amine 1074

Tan solid (20 mg, 0.046 mmol, 39.4% yield). ¹H NMR (499 MHz, DMSO-d₆) δ ppm 1.71 (2H, qd, J=11.98, 3.84 Hz), 1.96 (2H, br dd, J=12.08, 2.20 Hz), 1.99-2.06 (2H, m), 2.20 (3H, s), 2.85 (2H, br d, J=11.53 Hz), 3.89 (3H, s), 4.14-4.25 (1H, m), 7.74 (1H, d, J=8.51 Hz), 7.97-8.01 (3H, m), 8.21 (1H, dd, J=8.51, 1.92 Hz), 8.24 (1H, s), 8.43 (1H, s), 8.46 (1H, d, J=1.92 Hz), 8.56 (1H, d, J=1.92 Hz), 8.59 (1H, d, J=1.65 Hz), 12.02 (1H, br s); ESIMS found for C₂₅H₂₆N₈ m/z 439.1 (M+1).

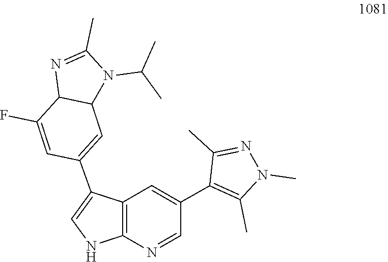 [SMILES: CC1=NC2C(F)=CC(c3c[nH]c4ncc(-c5c(C)nn(C)c5C)cc34)=CC2N1C(C)C]

### 4-Fluoro-1-isopropyl-2-methyl-6-(5-(1,3,5-trimethyl-1H-pyrazol-4-yl)-1H-pyrrolo[2,3-b]pyridin-3-yl)-1H-benzo[d]imidazole 1081

Off-white solid (15 mg, 0.036 mmol, 50.1% yield). ¹H NMR (499 MHz, DMSO-d₆) δ ppm 1.60 (6H, d, J=6.86 Hz), 2.17 (3H, s), 2.26 (3H, s), 2.58 (3H, s), 3.73 (3H, s), 4.80 (1H, spt, J=6.95 Hz), 7.29 (1H, d, J=12.08 Hz), 7.68 (1H, s), 7.97 (1H, s), 8.03 (1H, d, J=1.92 Hz), 8.18 (1H, d, J=1.65 Hz), 12.00 (1H, br s); ESIMS found for C₂₄H₂₅FN₆ m/z 417.15 (M+1).

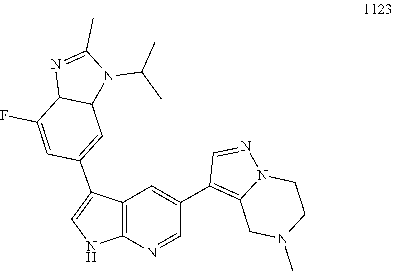 [SMILES: CC1=NC2C(F)=CC(c3c[nH]c4ncc(-c5cnn6c5CN(C)CC6)cc34)=CC2N1C(C)C]

### 3-(3-(4-Fluoro-1-isopropyl-2-methyl-1H-benzo[d]imidazol-6-yl)-1H-pyrrolo[2,3-b]pyridin-5-yl)-5-methyl-4,5,6,7-tetrahydropyrazolo[1,5-a]pyrazine 1123

Beige solid (10 mg, 0.023 mmol, 26.3% yield). ¹H NMR (499 MHz, DMSO-d₆) δ ppm 1.63 (6H, br d, J=5.49 Hz), 2.43 (3H, br s), 2.60 (3H, br s), 2.90 (2H, br s), 3.83 (2H, br s), 4.16 (2H, br s), 4.77-4.89 (1H, m), 7.31 (1H, br d, J=11.25 Hz), 7.70 (1H, br s), 7.84 (1H, br s), 7.94 (1H, br s), 8.11 (1H, br s), 8.37 (1H, br s), 11.96 (1H, br s); ESIMS found for C₂₅H₂₆FN₇ m/z 444.15 (M+1).

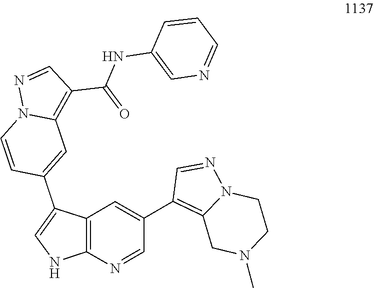 [SMILES: CN1CCn2ncc(-c3cnc4[nH]cc(-c5ccn6ncc(C(=O)Nc7cccnc7)c6c5)c4c3)c2C1]

### 5-(5-(5-Methyl-4,5,6,7-tetrahydropyrazolo[1,5-a]pyrazin-3-yl)-1H-pyrrolo[2,3-b]pyridin-3-yl)-N-(pyridin-3-yl)pyrazolo[1,5-a]pyridine-3-carboxamide 1137

Beige solid (4 mg, 0.008 mmol, 10.5% yield). ¹H NMR (499 MHz, DMSO-d₆) δ ppm 2.38 (3H, s), 2.91 (2H, t, J=5.48 Hz), 3.94 (2H, s), 4.18 (2H, t, J=5.61 Hz), 7.39 (1H, dd, J=8.21, 4.65 Hz), 7.60 (1H, dd, J=7.26, 2.05 Hz), 7.87 (1H, s), 8.26 (1H, dt, J=8.76, 1.78 Hz), 8.29 (1H, d, J=1.37 Hz), 8.31 (2H, dd, J=8.76, 1.92 Hz), 8.48 (1H, d, J=1.92 Hz), 8.68 (1H, d, J=1.64 Hz), 8.77 (1H, s), 8.83 (1H, d, J=7.39 Hz), 8.96 (1H, d, J=2.46 Hz), 10.12 (1H, s), 12.30 (1H, br s); ESIMS found for C₂₇H₂₃N₉O m/z 490.1 (M+1).

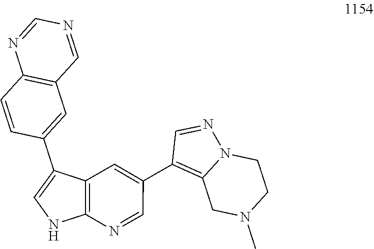 [SMILES: CN1CCn2ncc(-c3cnc4[nH]cc(-c5ccc6ncncc6c5)c4c3)c2C1]

### 6-(5-(5-Methyl-4,5,6,7-tetrahydropyrazolo[1,5-a]pyrazin-3-yl)-1H-pyrrolo[2,3-b]pyridin-3-yl)quinazoline 1154

Light orange solid (4 mg, 0.011 mmol, 12.5% yield). ¹H NMR (499 MHz, DMSO-d₆) δ ppm 2.45 (3H, s), 2.91 (2H, br t, J=5.48 Hz), 3.86 (2H, s), 4.18 (2H, br t, J=5.48 Hz), 7.93 (1H, s), 8.05 (1H, d, J=8.49 Hz), 8.19 (1H, d, J=2.46 Hz), 8.37 (1H, d, J=1.92 Hz), 8.45 (1H, d, J=1.64 Hz), 8.48 (1H, dd, J=8.90, 2.05 Hz), 8.52 (1H, d, J=1.92 Hz), 9.24 (1H, s), 9.68 (1H, s), 12.18 (1H, br s); ESIMS found for C₂₂H₁₉N₇ m/z 382.1 (M+1).

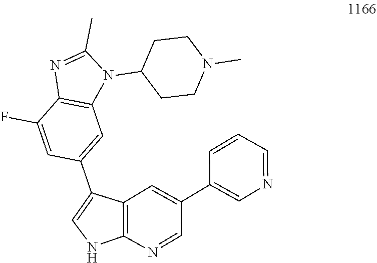 [SMILES: Cc1nc2c(F)cc(-c3c[nH]c4ncc(-c5cccnc5)cc34)cc2n1C1CCN(C)CC1]

### 4-Fluoro-2-methyl-1-(1-methylpiperidin-4-yl)-6-(5-(pyridin-3-yl)-1H-pyrrolo[2,3-b]pyridin-3-yl)-1H-benzo[d]imidazole 1166

Beige solid (14 mg, 0.032 mmol, 36.9% yield). ¹H NMR (499 MHz, DMSO-d₆) δ ppm 1.90 (2H, br dd, J=11.80, 2.47 Hz), 2.11-2.20 (2H, m), 2.28 (3H, s), 2.51-2.58 (2H, m), 2.61 (3H, s), 2.97 (2H, br d, J=11.25 Hz), 4.32-4.44 (1H, m), 7.40(1H, dd, J=11.94, 0.96 Hz), 7.49-7.55 (1H, m), 7.79 (1H, d, J=1.10 Hz), 8.05 (1H, s), 8.20 (1H, dt, J=7.96, 2.06 Hz), 8.51 (1H, d, J=2.20 Hz), 8.60 (1H, dd, J=4.67, 1.65 Hz), 8.66 (1H, d, J=1.92 Hz), 9.00-9.06 (1H, m), 12.13 (1H, s); ESIMS found for C₂₆H₂₅FN₆ m/z 441.2 (M+1).

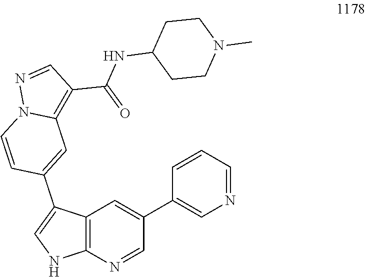 [SMILES: CN1CCC(NC(=O)c2cnn3ccc(-c4c[nH]c5ncc(-c6cccnc6)cc45)cc23)CC1]

### N-(1-Methylpiperidin-4-yl)-5-(5-(pyridin-3-yl)-1H-pyrrolo[2,3-b] pyridin-3-yl)pyrazolo[1,5-a]pyridine-3-carboxamide 1178

White solid (6 mg, 0.013 mmol, 13.2% yield). ¹H NMR (499 MHz, DMSO-d₆) δ ppm 1.57 (2H, qd, J=11.85, 3.43 Hz), 1.79 (2H, br d, J=10.70 Hz), 1.99 (2H, br t, J=10.98 Hz), 2.18 (3H, s), 2.78 (2H, br d, J=11.25 Hz), 3.73-3.84 (1H, m), 7.53-7.57 (2H, m), 7.94 (1H, d, J=7.96 Hz), 8.16-8.22 (1H, m), 8.26 (1H, d, J=1.92 Hz), 8.53 (1H, d, J=1.37 Hz), 8.55 (1H, s), 8.57 (1H, d, J=1.92 Hz), 8.62 (1H, br d, J=4.12 Hz), 8.66 (1H, d, J=1.92 Hz), 8.76 (1H, d, J=7.14 Hz), 8.99 (1H, s), 12.39 (1H, br s); ESIMS found for C₂₆H₂₅N₇O m/z 452.1 (M+1).

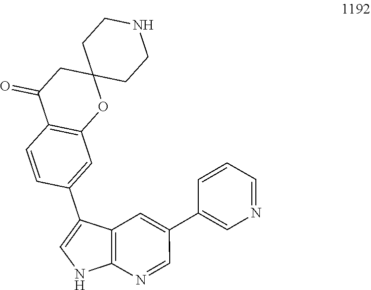 [SMILES: O=C1CC2(CCNCC2)Oc2cc(-c3c[nH]c4ncc(-c5cccnc5)cc34)ccc21]

### 7-(5-(Pyridin-3-yl)-1H-pyrrolo[2,3-b]pyridin-3-yl)spiro[chromane-2,4′-piperidin]-4-one 1192

Beige solid (6 mg, 0.015 mmol, 59.5% yield). ¹H NMR (499 MHz, DMSO-d₆) δ ppm 1.58-1.68 (2H, m), 1.87 (2H, br d, J=12.90 Hz), 2.73 (2H, br d, J=12.62 Hz), 2.80 (2H, s), 2.90 (2H, br t, J=10.57 Hz), 7.44 (1H, s), 7.53 (1H, br dd, J=7.55, 4.80 Hz), 7.57 (1H, br d, J=8.23 Hz), 7.78 (1H, br d, J=8.23 Hz), 8.17-8.26 (2H, m), 8.57 (1H, s), 8.60 (1H, br d, J=4.12 Hz), 8.63 (1H, s), 9.02 (1H, s), 12.33 (1H, br s); ESIMS found for C₂₅H₂₂N₄O₂ m/z 411.2 (M+1).

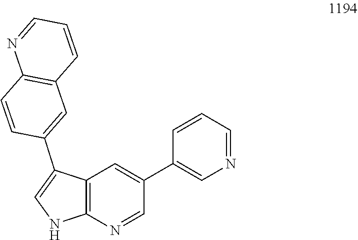 [SMILES: c1cncc(-c2cnc3[nH]cc(-c4ccc5ncccc5c4)c3c2)c1]

### 6-(5-(Pyridin-3-yl)-1H-pyrrolo[2,3-b]pyridin-3-yl)quinoline 1194

Off-white solid (15 mg, 0.047 mmol, 28.1% yield). ¹H NMR (500 MHz, DMSO-d₆) δ ppm 7.50-7.58 (2H, m), 8.06 (1H, d, J=8.78 Hz), 8.18 (1H, s), 8.23-8.27 (2H, m), 8.44 (1H, d, J=1.92 Hz), 8.50 (1H, dd, J=8.51, 1.10 Hz), 8.61 (1H, dd, J=4.67, 1.65 Hz), 8.66 (1H, d, J=1.92 Hz), 8.76 (1H, d, J=1.92 Hz), 8.84 (1H, dd, J=4.12, 1.65 Hz), 9.07 (1H, dd, J=2.47, 0.82 Hz), 12.23 (1H, s); ESIMS found for C₂₁H₁₄N₄ m/z 323.1 (M+1).

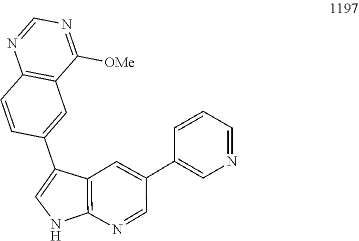 [SMILES: COc1ncnc2ccc(-c3c[nH]c4ncc(-c5cccnc5)cc34)cc12]

### 4-Methoxy-6-(5-(pyridin-3-yl)-1H-pyrrolo[2,3-b]pyridin-3-yl) quinazoline 1197

White solid (6 mg, 0.017 mmol, 10.3% yield). ¹H NMR (499 MHz, DMSO-d₆) δ ppm 4.17 (3H, s), 7.50-7.57 (1H, m), 7.99 (1H, d, J=8.76 Hz), 8.19-8.23 (2H, m), 8.40 (1H, d, J=1.64 Hz), 8.46 (1H, dd, J=8.62, 2.05 Hz), 8.58 (1H, d, J=1.92 Hz), 8.60 (1H, dd, J=4.65, 1.37 Hz), 8.66 (1H, d, J=2.19 Hz), 8.78 (1H, s), 9.02 (1H, d, J=1.92 Hz), 12.29 (1H, s); ESIMS found for C₂₁H₁₅N₅O m/z 354.05 (M+1).

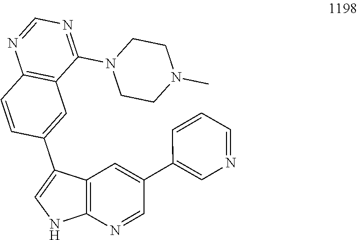 [SMILES: CN1CCN(c2ncnc3ccc(-c4c[nH]c5ncc(-c6cccnc6)cc45)cc23)CC1]

### 4-(4-Methylpiperazin-1-yl)-6-(5-(pyridin-3-yl)-1H-pyrrolo[2,3-b] pyridin-3-yl)quinazoline 1198

Light orange solid (5 mg, 0.012 mmol, 22.2% yield). ¹H NMR (499 MHz, DMSO-d₆) δ ppm 2.17 (3H, s), 2.52 (4H, br s), 3.71-3.80 (4H, m), 7.50-7.57 (1H, m), 7.88 (1H, d, J=8.76 Hz), 8.20 (1H, s), 8.22 (1H, dt, J=7.87, 1.95 Hz), 8.24 (1H, d, J=1.92 Hz), 8.31 (1H, dd, J=8.62, 2.05 Hz), 8.57 (1H, d, J=2.19 Hz), 8.60-8.63 (2H, m), 8.66 (1H, d, J=2.19 Hz), 9.02 (1H, d, J=2.46 Hz), 12.28 (1H, s); ESIMS found for C₂₅H₂₃N₇ m/z 422.1 (M+1).

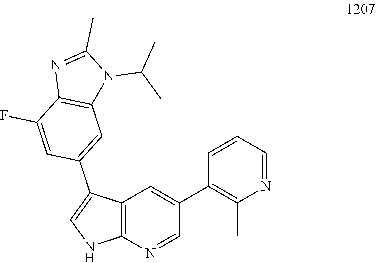 [SMILES: Cc1ncccc1-c1cnc2[nH]cc(-c3cc(F)c4nc(C)n(C(C)C)c4c3)c2c1]

### 4-Fluoro-1-isopropyl-2-methyl-6-(5-(2-methylpyridin-3-yl)-1H-pyrrolo[2,3-b]pyridin-3-yl)-1H-benzo[d]imidazole 1207

White solid (8 mg, 0.020 mmol, 38.6% yield). ¹H NMR (500 MHz, DMSO-d₆) δ ppm 1.59 (6H, d, J=6.86 Hz), 2.51 (3H, br s), 2.58 (3H, s), 4.80 (1H, spt, J=7.02 Hz), 7.31-7.36 (2H, m), 7.69 (1H, d, J=1.37 Hz), 7.76 (1H, dd, J=7.68, 1.65 Hz), 8.03 (1H, d, J=2.74 Hz), 8.23 (1H, d, J=1.92 Hz), 8.31 (1H, d, J=2.20 Hz), 8.49 (1H, dd, J=4.80, 1.78 Hz), 12.10 (1H, br s); ESIMS found for C₂₄H₂₂FN₅ m/z 400.2 (M+1).

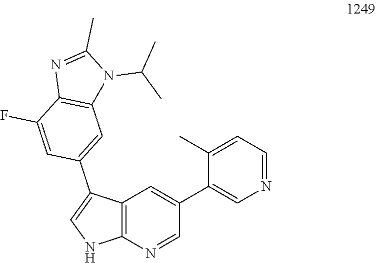 [SMILES: Cc1ccncc1-c1cnc2[nH]cc(-c3cc(F)c4nc(C)n(C(C)C)c4c3)c2c1]

### 4-Fluoro-1-isopropyl-2-methyl-6-(5-(4-methylpyridin-3-yl)-1H-pyrrolo[2,3-b]pyridin-3-yl)-1H-benzo[d]imidazole 1249

Beige solid (32 mg, 0.080 mmol, 72.0% yield). ¹H NMR (499 MHz, DMSO-d₆) δ ppm 1.59 (6H, d, J=6.86 Hz), 2.34 (3H, s), 2.58 (3H, s), 4.80 (1H, spt, J=6.93 Hz), 7.33 (1H, d, J=12.08 Hz), 7.38 (1H, d, J=4.94 Hz), 7.69 (1H, s), 8.03 (1H, d, J=2.47 Hz), 8.23 (1H, d, J=1.92 Hz), 8.31 (1 d, J=1.92 Hz), 8.46 (1 d, J=5.21 Hz), 8.52 (1H, s), 12.10 (1H, s); ESIMS found for C₂₄H₂₂FN₅ m/z 400.2 (M+1).

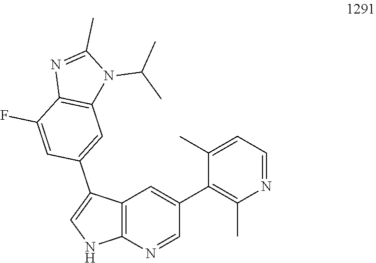 [SMILES: Cc1ccnc(C)c1-c1cnc2[nH]cc(-c3cc(F)c4nc(C)n(C(C)C)c4c3)c2c1]

### 6-(5-(2,4-Dimethylpyridin-3-yl)-1H-pyrrolo[2,3-b]pyridin-3-yl)-4-fluoro-1-isopropyl-2-methyl-1H-benzo[d]imidazole 1291

White solid (20 mg, 0.048 mmol, 44.6% yield). ¹H NMR (499 MHz, DMSO-d₆) δ ppm 1.58 (6H, d, J=6.86 Hz), 2.06 (3H, s), 2.24 (3H, s), 2.58 (3H, s), 4.78 (1H, spt, J=6.93 Hz), 7.21 (1H, d, J=5.21 Hz), 7.31 (1H, dd, J=11.94, 0.96 Hz), 7.65 (1H, d, J=1.10 Hz), 8.03 (1H, d, J=2.47 Hz), 8.08 (1H, d, J=1.65 Hz), 8.10 (1H, d, J=1.92 Hz), 8.34 (1H, d, J=5.21 Hz), 12.09 (1H, br s); ESIMS found for C₂₅H₂₄FN₅ m/z 414.2 (M+1).

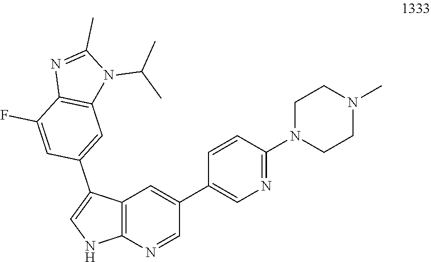 [SMILES: Cc1nc2c(F)cc(-c3c[nH]c4ncc(-c5ccc(N6CCN(C)CC6)nc5)cc34)cc2n1C(C)C]

### 4-Fluoro-1-isopropyl-2-methyl-6-(5-(6-(4-methylpiperazin-1-yl) pyridin-3-yl)-1H-pyrrolo[2,3-b]pyridin-3-yl)-1H-benzo[d]imidazole 1333

White solid (10 mg, 0.021 mmol, 17.2% yield). ¹H NMR (499 MHz, DMSO-d₆) δ ppm 1.62 (6H, d, J=6.86 Hz), 2.23 (3H, s), 2.39-2.45 (4H, m), 2.59 (3H, s), 3.51-3.57 (4H, m), 4.82 (1H, spt, J=6.93 Hz), 6.95 (1H, d, J=8.78 Hz), 7.36 (1H, d, J=12.08 Hz), 7.72 (1H, s), 7.93-7.97 (2H, m), 8.33 (1H, d, J=2.20 Hz), 8.52 (2H, t, J=2.33 Hz), 11.98 (1H, s); ESIMS found for C₂₈H₃₀FN₇ m/z 484.3 (M+1).

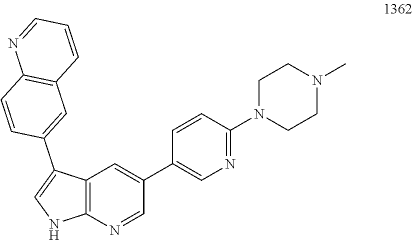 [SMILES: CN1CCN(c2ccc(-c3cnc4[nH]cc(-c5ccc6ncccc6c5)c4c3)cn2)CC1]

### 6-(5-(6-(4-Methylpiperazin-1-yl)pyridin-3-yl)-1H-pyrrolo[2,3-b] pyridin-3-yl)quinoline 1362

Beige solid (30 mg, 0.071 mmol, 53.3% yield). ¹H NMR (499 MHz, DMSO-d₆) δ ppm 2.23 (3H, s), 2.42 (4H, t, J=5.08 Hz), 3.52-3.58 (4H, m), 6.96 (1H, d, J=8.78 Hz), 7.53 (1H, dd, J=8.23, 4.12 Hz), 8.01 (1H, dd, J=8.78, 2.47 Hz), 8.05 (1H, d, J=8.78 Hz), 8.13 (1H, s), 8.23 (1H, dd, J=8.78, 1.92 Hz), 8.40 (1H, d, J=1.92 Hz), 8.49 (1H, d, J=7.68 Hz), 8.56 (1H, d, J=2.20 Hz), 8.58 (1H, d, J=2.47 Hz), 8.61 (1H, d, J=2.20 Hz), 8.84 (1H, dd, J=4.12, 1.65 Hz), 12.11 (1H, s); ESIMS found for C₂₆H₂₄N₆ m/z 421.2 (M+1).

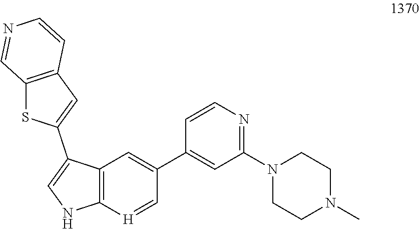

### 2-(5-(2-(4-Methylpiperazin-1-yl)pyridin-4-yl)-1H-pyrrolo[2,3-b] pyridin-3-yl)thieno[2,3-c]pyridine 1370

Light orange solid (4 mg, 0.009 mmol, 23.7% yield). ¹H NMR (499 MHz, DMSO-d₆) δ ppm 2.28 (4H, br s), 3.56-3.68 (4H, m), 7.13 (1H, d, J=4.93 Hz), 7.21 (1H, s), 7.74-7.81 (1H, m), 7.97 (1H, s), 8.23 (1H, br d, J=8.76 Hz), 8.23 (1H, s), 8.44 (1H, d, J=5.48 Hz), 8.68 (1H, d, J=1.92 Hz), 8.73 (1H, d, J=1.92 Hz), 9.17 (1H, s), 12.49 (1H, br s); ESIMS found for C₂₄H₂₂N₆S m/z 427.1 (M+1).

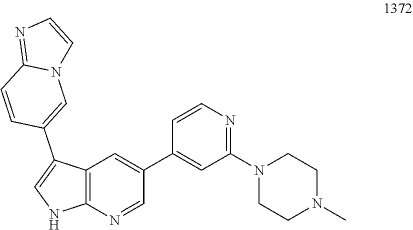 [SMILES: CN1CCN(c2cc(-c3cnc4[nH]cc(-c5ccc6nccn6c5)c4c3)ccn2)CC1]

### 6-(5-(2-(4-Methylpiperazin-1-yl)pyridin-4-yl)-1H-pyrrolo[2,3-b] pyridin-3-yl)imidazo[1,2-a]pyridine 1372

Tan solid (4 mg, 0.010 mmol, 15.3% yield). ¹H NMR (499 MHz, DMSO-d₆) δ ppm 2.23 (3H, s), 2.42 (4H, t, J=4.94 Hz), 3.55-3.62 (4H, m), 7.11 (1H, dd, J=5.21, 1.10 Hz), 7.18 (1H, s), 7.58 (1H, d, J=1.10 Hz), 7.62-7.66 (1H, m), 7.67-7.72 (1H, m), 8.01 (1H, s), 8.03 (1H, d, J=2.20 Hz), 8.20 (1H, d, J=5.21 Hz), 8.63 (1H, d, J=1.92 Hz), 8.68 (1H, d, J=2.20 Hz), 9.03 (1H, s), 12.16 (1H, s); ESIMS found for C₂₄H₂₃N₇ m/z 410.1 (M+1).

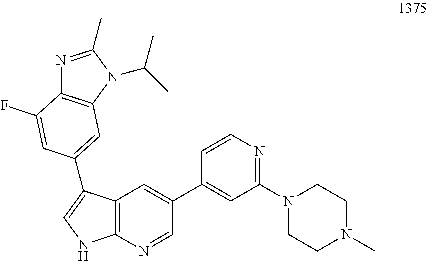 [SMILES: Cc1nc2c(F)cc(-c3c[nH]c4ncc(-c5ccnc(N6CCN(C)CC6)c5)cc34)cc2n1C(C)C]

### 4-Fluoro-1-isopropyl-2-methyl-6-(5-(2-(4-methylpiperazin-1-yl) pyridin-4-yl)-1H-pyrrolo[2,3-b]pyridin-3-yl)-1H-benzo[d]imidazole 1375

White solid (24 mg, 0.050 mmol, 48.3% yield). ¹H NMR (499 MHz, DMSO-d₆) δ ppm 1.63 (6H, d, J=6.86 Hz), 2.23 (3H, s), 2.41 (4H, t, J=4.94 Hz), 2.60 (3H, s), 3.54-3.61 (4H, m), 4.84 (1H, spt, J=6.93 Hz), 7.07 (1H, dd, J=5.21, 1.10 Hz), 7.20 (1H, s), 7.35 (1H, d, J=11.80 Hz), 7.78 (1H, d, J=0.82 Hz), 8.00 (1H, s), 8.18 (1H, d, J=4.94 Hz), 8.51 (1H, d, J=1.92 Hz), 8.67 (1H, d, J=1.92 Hz), 12.10 (1H, s); ESIMS found for C₂₈H₃₀FN₇ m/z 484.3 (M+1).

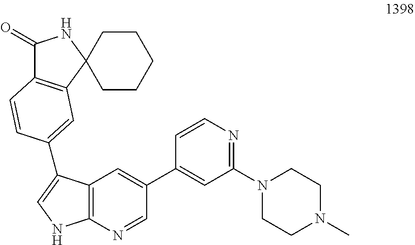 [SMILES: CN1CCN(c2cc(-c3cnc4[nH]cc(-c5ccc6c(c5)C5(CCCCC5)NC6=O)c4c3)ccn2)CC1]

### 6′-(5-(2-(4-Methylpiperazin-1-yl)pyridin-4-yl)-1H-pyrrolo[2,3-b] pyridin-3-yl)spiro[cyclohexane-1,1′-isoindolin]-3′-one 1398

Light yellow solid (20 mg, 0.041 mmol, 37.5% yield). ¹H NMR (500 MHz, DMSO-d₆) δ ppm 1.43 (3H, br d, J=12.08 Hz), 1.72 (5H, br s), 2.02-2.14 (2H, m), 2.22 (3H, s), 2.39-2.45 (4H, m), 3.56-3.65 (4H, m), 7.08 (1H, dd, J=5.21, 1.37 Hz), 7.22 (1H, s), 7.67 (1H, d, J=7.68 Hz), 7.86 (1H, dd, J=7.82, 1.51 Hz), 8.02 (1H, d, J=0.82 Hz), 8.08 (1H, s), 8.19 (1H, d, J=5.21 Hz), 8.55 (1H, d, J=2.20 Hz), 8.67 (1H, d, J=2.20 Hz), 9.07 (1H, s), 12.21 (1H, s); ESIMS found for C₃₀H₃₂N₆O m/z 493.2 (M+1).

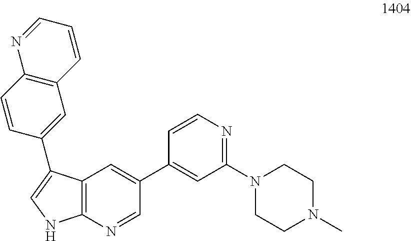 [SMILES: CN1CCN(c2cc(-c3cnc4[nH]cc(-c5ccc6ncccc6c5)c4c3)ccn2)CC1]

### 6-(5-(2-(4-Methylpiperazin-1-yl)pyridin-4-yl)-1H-pyrrolo[2,3-b] pyridin-3-yl)quinoline 1404

Off-white solid (15 mg, 0.036 mmol, 23.5% yield). ¹H NMR (499 MHz, DMSO-d₆) δ ppm 2.23 (3H, s), 2.42 (4H, t, J=4.94 Hz), 3.55-3.63 (4H, m), 7.12 (1H, dd, J=5.21, 1.10 Hz), 7.19 (1H, s), 7.54 (1H, dd, J=8.23, 4.12 Hz), 8.07 (1H, d, J=8.78 Hz), 8.16 (1H, s), 8.20 (1H, d, J=5.21 Hz), 8.23 (1H, dd, J=8.78, 2.20 Hz), 8.41 (1H, d, J=1.92 Hz), 8.45-8.52 (1H, m), 8.69 (1H, d, J=2.20 Hz), 8.72 (1H, d, J=1.92 Hz), 8.85 (1H, dd, J=4.25, 1.78 Hz), 12.24 (1H, br s); ESIMS found for C₂₆H₂₄N₆ m/z 421.2 (M+1).

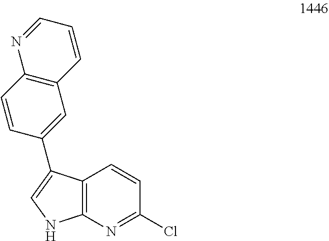 [SMILES: Clc1ccc2c(-c3ccc4ncccc4c3)c[nH]c2n1]

### 6-(6-Chloro-1H-pyrrolo[2,3-b]pyridin-3-yl)quinoline 1446

Beige solid (20 mg, 0.078 mmol, 10.9% yield). ¹H NMR (500 MHz, DMSO-d₆) δ ppm 7.28 (1H, d, J=8.23 Hz), 7.54 (1H, dd, J=8.23, 4.39 Hz), 8.05 (1H, d, J=8.78 Hz), 8.15 (1H, d, J=2.47 Hz), 8.17 (1H, dd, J=8.78, 2.20 Hz), 8.33 (1H, d, J=1.92 Hz), 8.44 (1H, dd, J=8.64, 0.96 Hz), 8.58 (1H, d, J=8.23 Hz), 8.85 (1H, dd, J=4.25, 1.78 Hz), 12.29 (1H, br s); ESIMS found for C₁₆H₁₀ClN₃ m/z 280.1 (M+1).

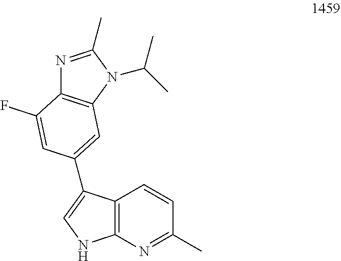 [SMILES: Cc1ccc2c(-c3cc(F)c4nc(C)n(C(C)C)c4c3)c[nH]c2n1]

### 4-Fluoro-1-isopropyl-2-methyl-6-(6-methyl-1H-pyrrolo[2,3-b]pyridin-3-yl)-1H-benzo[d]imidazole 1459

White solid (14 mg, 0.043 mmol, 13.1% yield). ¹H NMR (500 MHz, DMSO-d₆) δ ppm 1.61 (6H, d, J=6.86 Hz), 2.55 (3H, s), 2.59 (3H, s), 4.81 (1H, spt, J=6.90 Hz), 7.06 (1H, d, J=8.23 Hz), 7.27 (1H, dd, J=12.08, 1.10 Hz), 7.63 (1H, d, J=1.37 Hz), 7.83 (1H, d, J=2.47 Hz), 8.14 (1H, d, J=8.23 Hz), 11.71 (1H, br s); ESIMS found for C₁₉H₁₉FN₄ m/z 323.2 (M+1).

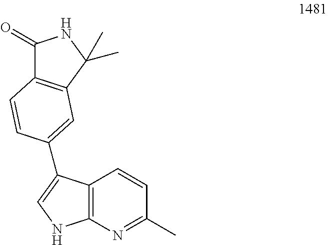 [SMILES: Cc1ccc2c(-c3ccc4c(c3)C(C)(C)NC4=O)c[nH]c2n1]

### 3,3-Dimethyl-5-(6-methyl-1H-pyrrolo[2,3-b]pyridin-3-yl)isoindolin-1-one 1481

White solid (36 mg, 0.124 mmol, 54.5% yield). ¹H NMR (499 MHz, DMSO-d₆) δ ppm 1.51 (6H, s), 2.55 (3H, s), 7.07 (1H, d, J=7.96 Hz), 7.62 (1H, d, J=7.96 Hz), 7.79 (1H, dd, J=7.96, 1.37 Hz), 7.90 (1H, s), 7.92 (1H, d, J=2.47 Hz), 8.25 (1H, d, J=8.23 Hz), 8.53 (1H, s), 11.84 (1H, br s); ESIMS found for C₁₈H₁₇N₃O m/z 292.1 (M+1).

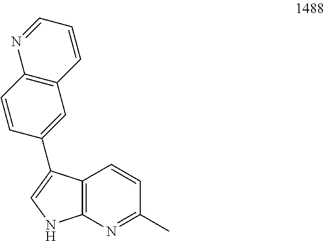 [SMILES: Cc1ccc2c(-c3ccc4ncccc4c3)c[nH]c2n1]

### 6-(6-Methyl-1H-pyrrolo[2,3-b]pyridin-3-yl)quinoline 1488

Pale yellow solid (52 mg, 0.201 mmol, 42.3% yield). ¹H NMR (499 MHz, DMSO-d₆) δ ppm 2.57 (3H, s), 7.10 (1H, d, J=8.23 Hz), 7.52 (1H, dd, J=8.23, 4.12 Hz), 8.00 (1H, d, J=2.20 Hz), 8.03 (1H, d, J=8.78 Hz), 8.17 (1H, dd, J=8.78, 2.20 Hz), 8.30 (1H, d, J=1.92 Hz), 8.40-8.45 (2H, m), 8.82 (1H, dd, J=4.12, 1.65 Hz), 11.85 (1H, br s); ESIMS found for C₁₇H₁₃N₃ m/z 260.1 (M+1).

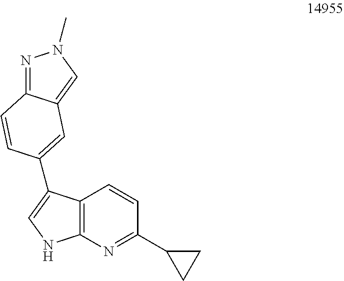 [SMILES: Cn1cc2cc(-c3c[nH]c4nc(C5CC5)ccc34)ccc2n1]

### 5-(6-Cyclopropyl-1H-pyrrolo[2,3-b]pyridin-3-yl)-2-methyl-2H-indazole 1495

Off-white solid (5 mg, 0.017 mmol, 25.6% yield). ¹H NMR (499 MHz, DMSO-d₆) δ ppm 0.93-0.99 (4H, m), 2.11-2.20 (1H, m), 7.09 (1H, d, J=8.23 Hz), 7.56-7.65 (2H, m), 7.68 (1H, d, J=2.74 Hz), 7.94 (1H, d, J=1.37 Hz), 8.18 (1H, d, J=8.23 Hz), 8.28 (1H, s), 11.60 (1H, br s); ESIMS found for C₁₈H₁₆N₄ m/z 289.05 (M+1).

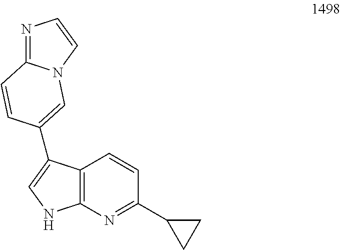 [SMILES: c1cn2cc(-c3c[nH]c4nc(C5CC5)ccc34)ccc2n1]

### 6-(6-Cyclopropyl-1H-pyrrolo[2,3-b]pyridin-3-yl)imidazo[1,2-a] pyridine 1498

Off-white solid (13.6 mg, 0.050 mmol, 45.2% yield). ¹H NMR (499 MHz, DMSO-d₆) δ ppm 0.92-1.01 (4H, m), 2.14-2.24 (1H, m), 7.14 (1H, d, J=8.23 Hz), 7.55 (1H, d, J=1.10 Hz), 7.57-7.64 (2H, m), 7.81 (1H, d, J=2.47 Hz), 7.99 (1H, s), 8.26 (1H, d, J=8.23 Hz), 8.91 (1H, d, J=1.37 Hz), 11.76 (1H, br s); ESIMS found for C₁₇H₁₄N₄ m/z 275.1 (M+1).

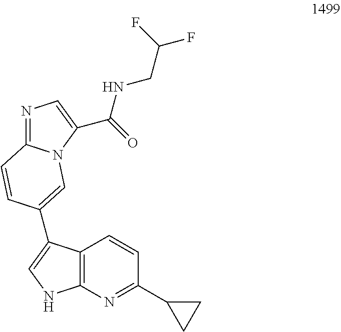 [SMILES: O=C(NCC(F)F)c1cnc2ccc(-c3c[nH]c4nc(C5CC5)ccc34)cn12]

### 6-(6-Cyclopropyl-1H-pyrrolo[2,3-b]pyridin-3-yl)-N-(2,2-difluoroethyl) imidazo[1,2-a]pyridine-3-carboxamide 1499

Beige solid (5 mg, 0.013 mmol, 17.6% yield). ¹H NMR (499 MHz, DMSO-d₆) δ ppm 0.93-1.02 (4H, m), 2.14-2.23 (1H, m), 3.76 (2H, tdd, J=15.67, 15.67, 5.61, 4.11 Hz), 6.18 (1H, tt, J=55.95, 3.90 Hz), 7.19 (1H, d, J=8.21 Hz), 7.78 (1H, d, J=9.31 Hz), 7.89 (1H, dd, J=9.31, 1.92 Hz), 7.93 (1H, d, J=2.74 Hz), 8.12 (1H, d, J=8.21 Hz), 8.41 (1H, s), 8.88 (1H, t, J=6.02 Hz), 9.88 (1H, d, J=0.82 Hz), 11.88 (1H, br d, J=1.37 Hz); ESIMS found for C₂₀H₁₇F₂N₅O m/z 382.2 (M+1).

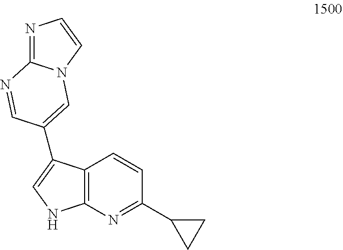 [SMILES: c1cn2cc(-c3c[nH]c4nc(C5CC5)ccc34)cnc2n1]

### 6-(6-Cyclopropyl-1H-pyrrolo[2,3-b]pyridin-3-yl)imidazo[1,2-a] pyrimidine 1500

Beige solid (20 mg, 0.073 mmol, 20.8% yield). ¹H NMR (499 MHz, DMSO-d₆) δ ppm 0.93-1.02 (4H, m), 2.15-2.25 (1H, m), 7.16 (1H, d, J=8.21 Hz), 7.70 (1H, d, J=1.10 Hz), 7.92 (1H, d, J=1.37 Hz), 7.98 (1H, d, J=2.74 Hz), 8.27 (1H, d, J=8.21 Hz), 8.96 (1H, d, J=2.74 Hz), 9.31 (1H, d, J=2.46 Hz), 11.89 (1H, br s); ESIMS found for C₁₆H₁₃N₅ m/z 276.1 (M+1).

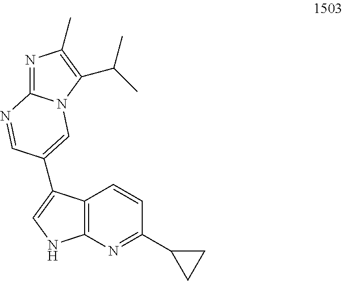 [SMILES: Cc1nc2ncc(-c3c[nH]c4nc(C5CC5)ccc34)cn2c1C(C)C]

### 6-(6-Cyclopropyl-1H-pyrrolo[2,3-b]pyridin-3-yl)-1-isopropyl-2-methyl-1H-imidazo[4,5-b]pyridine 1503

Brown solid (8.5 mg, 0.026 mmol, 25.4% yield). ¹H NMR (499 MHz, DMSO-d₆) δ ppm 0.92-0.99 (4H, m), 1.61 (6H, d, J=6.86 Hz), 2.13-2.22 (1H, m), 2.63 (3H, s), 4.81 (1H, spt, J=6.93 Hz), 7.11 (1H, d, J=7.96 Hz), 7.83 (1H, d, J=2.47 Hz), 8.12 (1H, d, J=8.23 Hz), 8.17 (1H, d, J=1.92 Hz), 8.65 (1H, d, J=1.92 Hz), 11.76 (1H, br s); ESIMS found for C₂₀H₂₁N₅ m/z 332.2 (M+1).

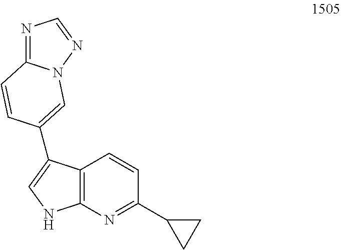 [SMILES: c1nc2ccc(-c3c[nH]c4nc(C5CC5)ccc34)cn2n1]

### 6-(6-Cyclopropyl-1H-pyrrolo[2,3-b]pyridin-3-yl)-[1,2,4]triazolo[1,5-a] pyridine 1505

Beige solid (6 mg, 0.022 mmol, 30.2% yield). ¹H NMR (499 MHz, DMSO-d₆) δ ppm 0.94-1.01 (4H, m), 2.14-2.23 (1H, m), 7.13 (1H, d, J=8.21 Hz), 7.90 (1H, dd, J=9.17, 0.96 Hz), 7.93 (1H, s), 8.07 (1H, dd, J=9.17, 1.78 Hz), 8.24 (1H, d, J=8.21 Hz), 8.49 (1H, s), 9.17 (1H, dd, J=1.64, 0.82 Hz), 11.89 (1H, br s); ESIMS found for C₁₆H₁₃N₅ m/z 276.1 (M+1).

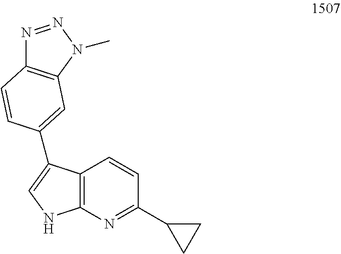 [SMILES: Cn1nnc2ccc(-c3c[nH]c4nc(C5CC5)ccc34)cc21]

### 6-(6-Cyclopropyl-1H-pyrrolo[2,3-b]pyridin-3-yl)-1-methyl-1H-benzo[d][1,2,3]triazole 1507

Off-white solid (20 mg, 0.069 mmol, 27.4% yield). ¹H NMR (499 MHz, DMSO-d₆) δ ppm 0.93-1.01 (4H, m), 2.15-2.23 (1H, m), 4.36 (3H, s), 7.15 (1H, d, J=8.21 Hz), 7.78 (1H, dd, J=8.62, 1.51 Hz), 7.90 (1H, d, J=2.74 Hz), 8.02 (1H, d, J=8.76 Hz), 8.04 (1H, s), 8.36 (1H, d, J=8.21 Hz), 11.84 (1H, br s); ESIMS found for C₁₇H₁₅N₅ m/z 290.1 (M+1).

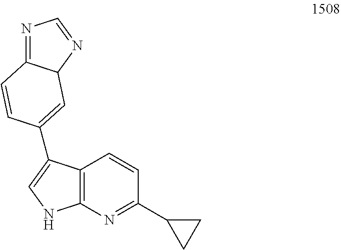 [SMILES: C1=CC2=NC=NC2C=C1c1c[nH]c2nc(C3CC3)ccc12]

### 6-(6-Cyclopropyl-1H-pyrrolo[2,3-b]pyridin-3-yl)imidazo[1,2-b] pyridazine 1508

Off-white solid (3.3 mg, 0.012 mmol, 23.4% yield). ¹H NMR (499 MHz, DMSO-d₆) δ ppm 0.94-1.01 (4H, m), 2.13-2.24 (1H, m), 7.20 (1H, d, J=7.96 Hz), 7.68 (1H, d, J=1.10 Hz), 7.78 (1H, d, J=9.61 Hz), 8.05 (1H, d, J=9.61 Hz), 8.28 (1H, s), 8.33 (1H, s), 8.64 (1H, d, J=8.23 Hz), 12.08 (1H, br s); ESIMS found for C₁₆H₁₃N₅ m/z 276.1 (M+1).

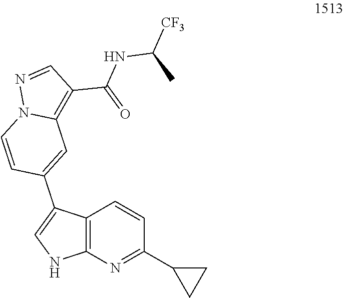 [SMILES: C[C@@H](NC(=O)c1cnn2ccc(-c3c[nH]c4nc(C5CC5)ccc34)cc12)C(F)(F)F]

### (R)-5-(6-Cyclopropyl-1H-pyrrolo[2,3-b]pyridin-3-yl)-N-(1,1,1-trifluoropropan-2-yl)pyrazolo[1,5-a]pyridine-3-carboxamide 1513

Beige solid (11 mg, 0.027 mmol, 12.1% yield). ¹H NMR (499 MHz, DMSO-d₆) δ ppm 0.95-1.02 (4H, m), 1.39 (3H, d, J=7.12 Hz), 2.15-2.24 (1H, m), 4.94 (1H, dq, J=15.61, 7.76 Hz), 7.23 (1H, d, J=8.21 Hz), 7.50 (1H, dd, J=7.26, 2.05 Hz), 8.11 (1H, d, J=2.19 Hz), 8.19 (1H, d, J=8.21 Hz), 8.50-8.53 (2H, m), 8.64 (1H, s), 8.76 (1H, d, J=7.67 Hz), 12.04 (1H, br s); ESIMS found for C₂₁H₁₈F₃N₅O m/z 414.2 (M+1).

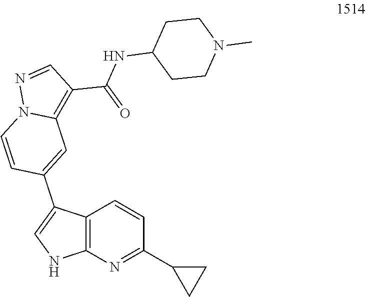 [SMILES: CN1CCC(NC(=O)c2cnn3ccc(-c4c[nH]c5nc(C6CC6)ccc45)cc23)CC1]

### 5-(6-Cyclopropyl-1H-pyrrolo[2,3-b]pyridin-3-yl)-N-(1-methylpiperidin-4-yl)pyrazolo[1,5-a]pyridine-3-carboxamide 1514

Beige solid (50 mg, 0.121 mmol, 44.6% yield). ¹H NMR (499 MHz, DMSO-d₆) δ ppm 0.94-1.03 (4H, m), 1.58 (2H, qd, J=12.00, 3.70 Hz), 1.77-1.85 (2H, m), 1.91-2.01 (2H, m), 2.16-2.22 (1H, m), 2.18 (3H, s), 2.80 (2H, br d, J=11.50 Hz), 3.73-3.85 (1H, m), 7.22 (1H, d, J=8.21 Hz), 7.44 (1H, dd, J=7.39, 2.19 Hz), 7.94 (1H, d, J=7.67 Hz), 8.07 (1H, d, J=2.74 Hz), 8.19 (1H, d, J=8.21 Hz), 8.53 (1H, d, J=1.64 Hz), 8.54 (1H, s), 8.67-8.73 (1H, m), 12.00 (1H, br s); ESIMS found for C₂₄H₂₆N₆O m/z 415.2 (M+1).

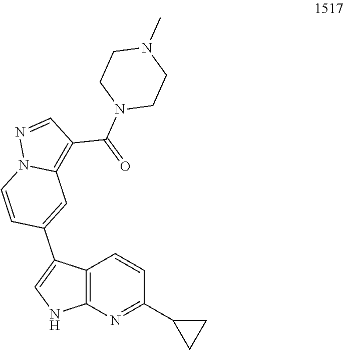 [SMILES: CN1CCN(C(=O)c2cnn3ccc(-c4c[nH]c5nc(C6CC6)ccc45)cc23)CC1]

### (5-(6-Cyclopropyl-1H-pyrrolo[2,3-b]pyridin-3-yl)pyrazolo[1,5-a] pyridin-3-yl)(4-methylpiperazin-1-yl)methanone 1517

Off-white solid (4.5 mg, 0.011 mmol, 8.0% yield). ¹H NMR (500 MHz, DMSO-d₆) δ ppm 0.92-1.02 (4H, m), 2.16-2.21 (1H, m), 2.22 (3H, s), 2.35-2.41 (4H, m), 3.65-3.72 (4H, m), 7.21 (1H, d, J=8.23 Hz), 7.45 (1H, dd, J=7.41, 1.92 Hz), 8.08 (1H, d, J=1.92 Hz), 8.14 (1H, d, J=1.65 Hz), 8.17 (1H, d, J=8.23 Hz), 8.22 (1H, s), 8.74 (1H, d, J=7.41 Hz), 12.00 (1H, br s); ESIMS found for C₂₃H₂₄N₆O m/z 401.3 (M+1).

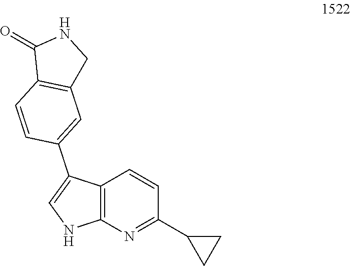 [SMILES: O=C1NCc2cc(-c3c[nH]c4nc(C5CC5)ccc34)ccc21]

### 5-(6-Cyclopropyl-1H-pyrrolo[2,3-b]pyridin-3-yl)isoindolin-1-one 1522

Off-white solid (12 mg, 0.042 mmol, 36.8% yield). ¹H NMR (499 MHz, DMSO-d₆) δ ppm 0.92-1.00 (4H, m), 2.13-2.22 (1H, m), 4.42 (2H, s), 7.13 (1H, d, J=8.23 Hz), 7.68 (1H, d, J=7.96 Hz), 7.79-7.83 (1H, m), 7.86 (1H, d, J=2.20 Hz), 7.89 (1H, s), 8.23 (1H, d, J=8.23 Hz), 8.44 (1H, s), 11.83 (1H, br s); ESIMS found for C₁₈H₁₅N₃O m/z 290.0 (M+1).

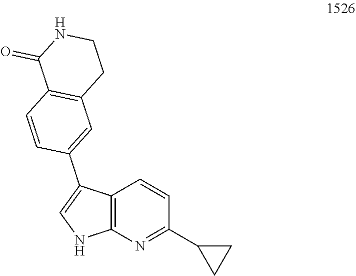 [SMILES: O=C1NCCc2cc(-c3c[nH]c4nc(C5CC5)ccc34)ccc21]

### 6-(6-Cyclopropyl-1H-pyrrolo[2,3-b]pyridin-3-yl)-3,4-dihydroisoquinolin-1(2H)-one 1526

Pale yellow solid (19.6 mg, 0.065 mmol, 28.4% yield). ¹H NMR (499 MHz, DMSO-d₆) δ ppm 0.92-1.00 (4H, m), 2.13-2.22 (1H, m), 2.97 (2H, t, J=6.59 Hz), 3.40 (2H, td, J=6.52, 2.61 Hz), 7.12 (1H, d, J=8.23 Hz), 7.64 (1H, s), 7.68 (1H, dd, J=7.96, 1.37 Hz), 7.83 (1H, br s), 7.86 (1H, br d, J=11.25 Hz), 7.85 (1H, s), 8.22 (1H, d, J=8.23 Hz), 11.82 (1H, br s); ESIMS found for C₁₉H₁₇N₃O m/z 304.1 (M+1).

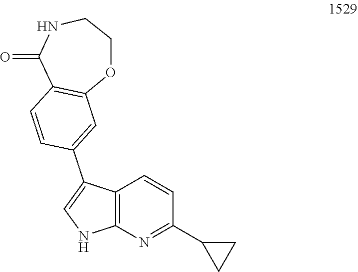 [SMILES: O=C1NCCOc2cc(-c3c[nH]c4nc(C5CC5)ccc34)ccc21]

### 8-(6-Cyclopropyl-1H-pyrrolo[2,3-b]pyridin-3-yl)-3,4-dihydrobenzo[f][1,4] oxazepin-5(2H)-one 1529

Off-white solid (18 mg, 0.056 mmol, 25.9% yield). ¹H NMR (499 MHz, DMSO-d₆) δ ppm 0.91-0.99(4H, m), 2.12-2.21 (1H, m), 3.34-3.40 (2H, m), 4.31-4.37 (2H, m), 7.11 (1H, d, J=8.23 Hz), 7.31 (1H, d, J=1.37 Hz), 7.48 (1H, dd, J=8.23, 1.65 Hz), 7.84 (1H, d, J=8.23 Hz), 7.87 (1H, d, J=1.92 Hz), 8.17 (1H, d, J=8.23 Hz), 8.25 (1H, br t, J=4.94 Hz), 11.83 (1H, br s); ESIMS found for C₁₉H₁₇N₃O₂ m/z 320.1 (M+1).

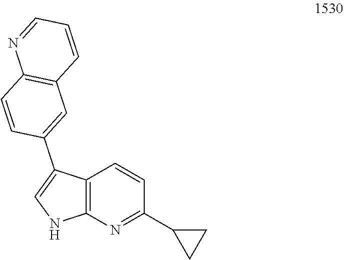 [SMILES: c1cnc2ccc(-c3c[nH]c4nc(C5CC5)ccc34)cc2c1]

### 6-(6-Cyclopropyl-1H-pyrrolo[2,3-b]pyridin-3-yl)quinoline 1530

White solid (13 mg, 0.046 mmol, 69.2% yield). ¹H NMR (499 MHz, DMSO-d₆) δ ppm 0.95-1.02 (4H, m), 2.20 (1H, quin, J=6.45 Hz), 7.16 (1H, d, J=8.23 Hz), 7.52 (1H, dd, J=8.23, 4.12 Hz), 7.95 (1H, s), 8.02 (1H, d, J=8.78 Hz), 8.16 (1H, dd, J=8.78, 2.20 Hz), 8.29 (1H, d, J=1.92 Hz), 8.38 (1H, d, J=8.23 Hz), 8.41-8.46 (1H, m), 8.82 (1H, dd, J=4.12, 1.65 Hz), 11.84 (1H, br s); ESIMS found for C₁₉H₁₅N₃ m/z 286.1 (M+1).

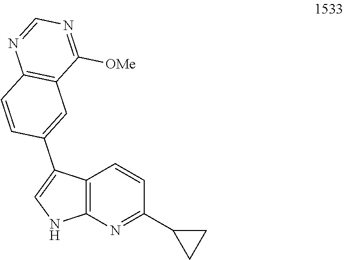 [SMILES: COc1ncnc2ccc(-c3c[nH]c4nc(C5CC5)ccc34)cc12]

### 6-(6-Cyclopropyl-1H-pyrrolo[2,3-b]pyridin-3-yl)-4-methoxyquinazoline 1533

Brown solid ((4.3 mg, 0.014 mmol, 21.3% yield). ¹HNMR (499 MHz, DMSO-d₆) δ ppm 0.96-0.99 (4H, m), 2.14-2.24 (1H, m), 4.18 (3H, s), 7.17 (1H, d, J=7.96 Hz), 7.95 (1H, d, J=9.33 Hz), 7.99 (1H, s), 8.20 (1H, d, J=8.23 Hz), 8.32-8.35 (2H, m), 8.76 (1H, s), 11.91 (1H, br s); ESIMS found for C₁₉H₁₆N₄O m/z 317.1 (M+1).

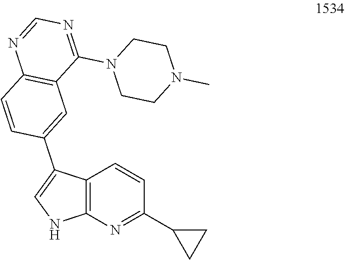 [SMILES: CN1CCN(c2ncnc3ccc(-c4c[nH]c5nc(C6CC6)ccc45)cc23)CC1]

### 6-(6-Cyclopropyl-1H-pyrrolo[2,3-b]pyridin-3-yl)-4-(4-methylpiperazin-1-yl)quinazoline 1534

Off-white solid (4.7 mg, 0.012 mmol, 14.0% yield). ¹H NMR (500 MHz, DMSO-d₆) δ ppm 0.92-1.03 (4H, m), 2.15-2.23 (1H, m), 2.27 (3H, s), 2.53-2.59 (4H, m), 3.72-3.83 (4H, m), 7.18 (1H, d, J=8.23 Hz), 7.85 (1H, d, J=8.78 Hz), 7.92 (1H, d, J=2.47 Hz), 8.11 (1H, d, J=1.92 Hz), 8.15 (1H, d, J=8.23 Hz), 8.19 (1H, dd, J=8.64, 1.78 Hz), 8.60 (1H, s), 11.87 (1H, br s); ESIMS found for C₂₃H₂₄N₆ m/z 385.2 (M+1).

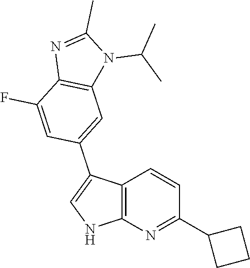 [SMILES: Cc1nc2c(F)cc(-c3c[nH]c4nc(C5CCC5)ccc34)cc2n1C(C)C]

### 6-(6-Cyclobutyl-1H-pyrrolo[2,3-b]pyridin-3-yl)-4-fluoro-1-isopropyl-2-methyl-1H-benzo[d]imidazole 1543

White solid (36 mg, 0.099 mmol, 33.3% yield). ¹H NMR (500 MHz, DMSO-d₆) δ ppm 1.61 (6H, d, J=6.86 Hz), 1.81-1.92 (1H, m), 1.96-2.10 (1H, m), 2.25-2.41 (4H, m), 2.56-2.62 (3H, m), 3.74 (1H, quin, J=8.58 Hz), 4.81 (1H, spt, J=6.96 Hz), 7.06 (1H, d, J=7.96 Hz), 7.27 (1H, dd, J=12.08, 1.10 Hz), 7.64 (1H, d, J=1.10 Hz), 7.85 (1H, d, J=2.47 Hz), 8.16 (1H, d, J=8.23 Hz), 11.83 (1H, br d, J=0.82 Hz); ESIMS found for C₂₂H₂₃FN₄ m/z 363.2 (M+1).

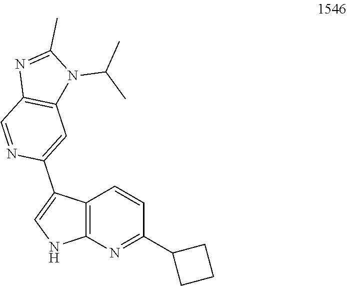 [SMILES: Cc1nc2cnc(-c3c[nH]c4nc(C5CCC5)ccc34)cc2n1C(C)C]

### 6-(6-Cyclobutyl-1H-pyrrolo[2,3-b]pyridin-3-yl)-1-isopropyl-2-methyl-1H-imidazo[4,5-c]pyridine 1546

Off-white solid (55 mg, 0.159 mmol, 77.3% yield). ¹H NMR (499 MHz, DMSO-d₆) δ ppm 1.62 (6H, d, J=7.12 Hz), 1.81-1.93 (1H, m), 1.96-2.09 (1H, m), 2.26-2.41 (4H, m), 2.59 (3H, s), 3.72 (1H, quin, J=8.83 Hz), 4.78 (1H, spt, J=6.94 Hz), 7.03 (1H, d, J=8.21 Hz), 8.00 (1H, d, J=1.09 Hz), 8.21 (1H, d, J=2.46 Hz), 8.68 (1H, d, J=8.21 Hz), 8.81 (1H, d, J=0.82 Hz), 11.81 (1H, br s); ESIMS found for C₂₁H₂₃N₅ m/z 346.2 (M+1).

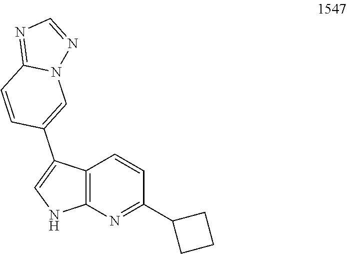 [SMILES: c1nc2ccc(-c3c[nH]c4nc(C5CCC5)ccc34)cn2n1]

### 6-(6-Cyclobutyl-1H-pyrrolo[2,3-b]pyridin-3-yl)-[1,2,4]triazolo[1,5-a] pyridine 1547

Beige solid (15 mg, 0.052 mmol, 31.8% yield). ¹H NMR (499 MHz, DMSO-d₆) δ ppm 1.82-1.92 (1H, m), 1.97-2.11 (1H, m), 2.25-2.39 (4H, m), 3.75 (1H, quin, J=8.69 Hz), 7.07 (1H, d, J=8.21 Hz), 7.91 (1H, dd, J=9.03, 0.82 Hz), 8.01 (1H, d, J=2.74 Hz), 8.08 (1H, dd, J=9.31, 1.92 Hz), 8.28 (1H, d, J=7.94 Hz), 8.49 (1H, s), 9.19 (1H, dd, J=1.64, 0.82 Hz), 12.00 (1H, br s); ESIMS found for C₁₇H₁₅N₅ m/z 290.1 (M+1).

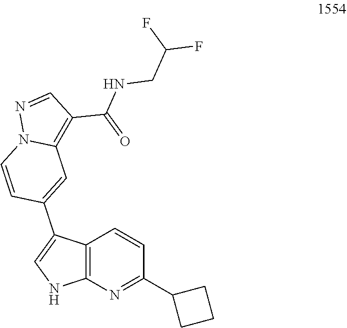 [SMILES: O=C(NCC(F)F)c1cnn2ccc(-c3c[nH]c4nc(C5CCC5)ccc34)cc12]

### 5-(6-Cyclobutyl-1H-pyrrolo[2,3-b]pyridin-3-yl)-N-(2,2-difluoroethyl) pyrazolo[1,5-a]pyridine-3-carboxamide 1554

Beige solid (6 mg, 0.015 mmol, 7.6% yield). ¹H NMR (499 MHz, DMSO-d₆) δ ppm 1.82-1.94 (1H, m), 1.96-2.11 (1H, m), 2.27-2.40 (4H, m), 3.65-3.81 (3H, m), 6.15 (1H, tt, J=56.20, 4.10 Hz), 7.16 (1H, d, J=8.21 Hz), 7.50 (1H, dd, J=7.26, 2.05 Hz), 8.17 (1H, d, J=2.74 Hz), 8.24 (1H, d, J=7.94 Hz), 8.53 (1H, d, J=1.37 Hz), 8.55-8.59 (2H, m), 8.74-8.80 (1H, m), 12.15 (1H, br d, J=1.92 Hz); ESIMS found for C₂₁H₁₉F₂N₅O m/z 396.2 (M+1).

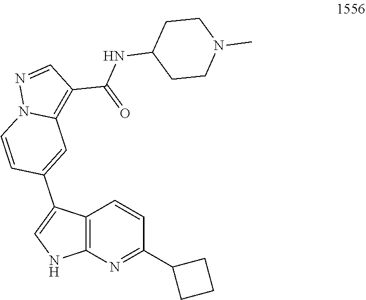 [SMILES: CN1CCC(NC(=O)c2cnn3ccc(-c4c[nH]c5nc(C6CCC6)ccc45)cc23)CC1]

### 5-(6-Cyclobutyl-1H-pyrrolo[2,3-b]pyridin-3-yl)-N-(1-methylpiperidin-4-yl)pyrazolo[1,5-a]pyridine-3-carboxamide 1556

Beige solid (50 mg, 0.117 mmol, 69.8% yield). ¹H NMR (499 MHz, DMSO-d₆) δ ppm 1.58 (2H, qd, J=11.96, 3.56 Hz), 1.76-1.84 (2H, m), 1.84-1.91 (1H, m), 1.95 (2H, td, J=11.77, 1.64 Hz), 1.99-2.09 (1H, m), 2.17 (3H, s), 2.26-2.40 (4H, m), 2.79 (2H, br d, J=11.50 Hz), 3.71-3.83 (2H, m), 7.15 (1H, d, J=8.21 Hz), 7.45 (1H, dd, J=7.26, 2.05 Hz), 7.95 (1H, d, J=7.94 Hz), 8.14 (1H, d, J=1.92 Hz), 8.24 (1H, d, J=8.21 Hz), 8.54 (1H, d, J=1.92 Hz), 8.55 (1H, s), 8.71 (1H, d, J=7.39 Hz), 12.12 (1H, br s); ESIMS found for C₂₅H₂₈N₆O m/z 429.2 (M+1).

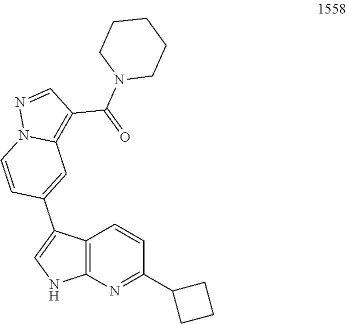 [SMILES: O=C(c1cnn2ccc(-c3c[nH]c4nc(C5CCC5)ccc34)cc12)N1CCCCC1]

### (5-(6-Cyclobutyl-1H-pyrrolo[2,3-b]pyridin-3-yl)pyrazolo[1,5-a] pyridin-3-yl)(piperidin-1-yl)methanone 1558

Beige solid (42 mg, 0.105 mmol, 64.7% yield). ¹H NMR (499 MHz, DMSO-d₆) δ ppm 1.53-1.61 (4H, m), 1.62-1.70 (2H, m), 1.81-1.92 (1H, m), 1.94-2.10 (1H, m), 2.27-2.40 (4H, m), 3.60-3.68 (4H, m), 3.75 (1H, quin, J=8.69 Hz), 7.14 (1H, d, J=8.21 Hz), 7.45 (1H, dd, J=7.26, 2.05 Hz), 8.13 (2H, t, J=2.19 Hz), 8.18 (1H, s), 8.21 (1H, d, J=8.21 Hz), 8.75 (1H, d, J=7.39 Hz), 12.11 (1H, br s); ESIMS found for C₂₄H₂₅N₅O m/z 400.2 (M+1).

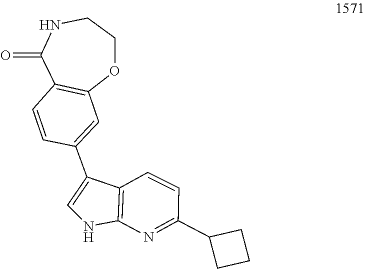 [SMILES: O=C1NCCOc2cc(-c3c[nH]c4nc(C5CCC5)ccc34)ccc21]

### 8-(6-Cyclobutyl-1H-pyrrolo[2,3-b]pyridin-3-yl)-3,4-dihydrobenzo[f] [1,4]oxazepin-5(2H)-one 1571

Brown solid (15 mg, 0.045 mmol, 5.4% yield). ¹H NMR (499 MHz, DMSO-d₆) δ ppm 1.80-1.92 (1H, m), 1.96-2.08 (1H, m), 2.25-2.39 (4H, m), 3.35-3.40 (2H, m), 3.73 (1H, quin, J=8.62 Hz), 4.34 (2H, br t, J=4.24 Hz), 7.05 (1H, d, J=8.21 Hz), 7.32 (1H, s), 7.49 (1H, br d, J=8.21 Hz), 7.85 (1H, br d, J=8.21 Hz), 7.94 (1H, d, J=1.92 Hz), 8.21 (1H, br d, J=8.21 Hz), 8.25 (1H, br s), 11.95 (1H, br s); ESIMS found for C₂₀H₁₉N₃O₂ m/z 334.1 (M+1).

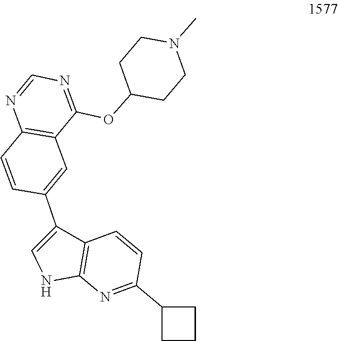 [SMILES: CN1CCC(Oc2ncnc3ccc(-c4c[nH]c5nc(C6CCC6)ccc45)cc23)CC1]

### 6-(6-Cyclobutyl-1H-pyrrolo[2,3-b]pyridin-3-yl)-4-((1-methylpiperidin-4-yl)oxy)quinazoline 1577

Beige solid (30 mg, 0.073 mmol, 11.7% yield). ¹H NMR (499 MHz, DMSO-d₆) δ ppm 1.84-1.97 (3H, m), 2.05-2.12 (2H, m), 2.23 (3H, s), 2.28-2.43 (7H, m), 2.64 (2H, br s), 3.76 (1H, quin, J=8.69 Hz), 5.42 (1H, dt, J=7.19, 3.66 Hz), 7.11 (1H, d, J=7.94 Hz), 7.94 (1H, d, J=8.76 Hz), 8.06 (1H, s), 8.24 (1H, d, J=8.21 Hz), 8.33 (1H, dd, J=8.62, 2.05 Hz), 8.36 (1H, d, J=1.92 Hz), 8.73 (1H, s), 12.05 (1H, br s); ESIMS found for C₂₅H₂₇N₅O m/z 414.1 (M+1).

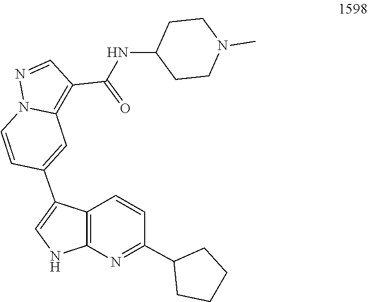 [SMILES: CN1CCC(NC(=O)c2cnn3ccc(-c4c[nH]c5nc(C6CCCC6)ccc45)cc23)CC1]

### 5-(6-Cyclopentyl-1H-pyrrolo[2,3-b]pyridin-3-yl)-N-(1-methylpiperidin-4-yl)pyrazolo[1,5-a]pyridine-3-carboxamide 1598

Beige solid (18 mg, 0.041 mmol, 59.3% yield). ¹H NMR (499 MHz, DMSO-d₆) δ ppm 1.58 (2H, qd, J=11.91, 3.70 Hz), 1.65-1.74 (2H, m), 1.76-1.89 (6H, m), 1.95 (2H, td, J=11.70, 1.78 Hz), 2.02-2.11 (2H, m), 2.18 (3H, s), 2.79 (2H, br d, J=11.77 Hz), 3.23-3.31 (1H, m), 3.73-3.84 (1H, m), 7.20 (1H, d, J=8.21 Hz), 7.45 (1H, dd, J=7.26, 2.05 Hz), 7.94 (1H, d, J=7.67 Hz), 8.12 (1H, d, J=2.46 Hz), 8.23 (1H, d, J=8.21 Hz), 8.54 (1H, d, J=1.37 Hz), 8.55 (1H, s), 8.71 (1H, dd, J=7.39, 0.82 Hz), 12.07 (1H, br s); ESIMS found for C₂₆H₃₀N₆O m/z 443.3 (M+1).

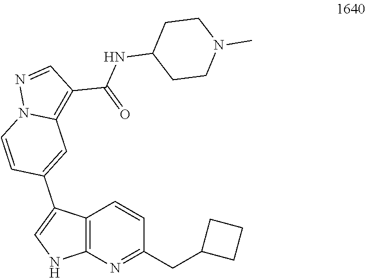 [SMILES: CN1CCC(NC(=O)c2cnn3ccc(-c4c[nH]c5nc(CC6CCC6)ccc45)cc23)CC1]

### 5-(6-(Cyclobutylmethyl)-1H-pyrrolo[2,3-b]pyridin-3-yl)-N-(1-methylpiperidin-4-yl)pyrazolo[1,5-a]pyridine-3-carboxamide 1640

Beige solid (18 mg, 0.041 mmol, 79.0% yield). ¹H NMR (499 MHz, DMSO-d₆) δ ppm 1.58 (2H, qd, J=12.05, 3.83 Hz), 1.74-1.87 (6H, m), 1.93-2.05 (4H, m), 2.18 (3H, s), 2.71-2.81 (3H, m), 2.92 (2H, d, J=7.67 Hz), 3.73-3.85 (1H, m), 7.13 (1H, d, J=7.94 Hz), 7.45 (1H, dd, J=7.26, 2.05 Hz), 7.94 (1H, d, J=7.67 Hz), 8.12 (1H, d, J=2.74 Hz), 8.22 (1H, d, J=8.21 Hz), 8.53 (1H, d, J=1.92 Hz), 8.55 (1H, s), 8.71 (1H, d, J=7.39 Hz), 12.04 (1H, br d, J=1.64 Hz); ESIMS found for C₂₆H₃₀N₆O m/z 443.25 (M+1).

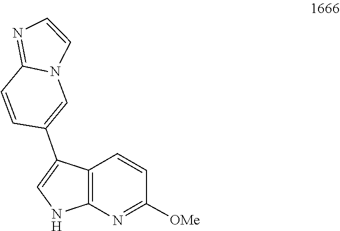 [SMILES: COc1ccc2c(-c3ccc4nccn4c3)c[nH]c2n1]

### 6-(6-Methoxy-1H-pyrrolo[2,3-b]pyridin-3-yl)imidazo[1,2-a]pyridine 1666

Beige solid (50 mg, 0.189 mmol, 21.5% yield). ¹H NMR (499 MHz, DMSO-d₆) δ ppm 3.90 (3H, s), 6.66 (1H, d, J=8.78 Hz), 7.55 (1H, d, J=1.10 Hz), 7.57-7.64 (2H, m), 7.70 (1H, d, J=2.47 Hz), 7.97 (1H, s), 8.27-8.37 (1H, m), 8.90 (1H, t, J=1.24 Hz), 11.80(1H, br s); ESIMS found for C₁₅H₁₂N₄O m/z 265.0 (M+1).

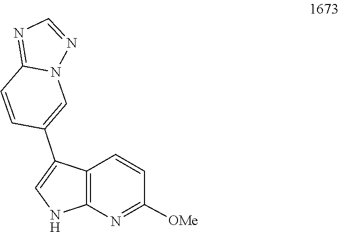 [SMILES: COc1ccc2c(-c3ccc4ncnn4c3)c[nH]c2n1]

### 6-(6-Methoxy-1H-pyrrolo[2,3-b]pyridin-3-yl)-[1,2,4]triazolo[1,5-a] pyridine 1673

Beige solid (20 mg, 0.075 mmol, 26.3% yield). ¹H NMR (499 MHz, DMSO-d₆) δ ppm 3.90 (3H, s), 6.65 (1H, d, J=8.76 Hz), 7.81 (1H, d, J=1.64 Hz), 7.89 (1H, dd, J=9.31, 0.82 Hz), 8.06 (1H, dd, J=9.31, 1.92 Hz), 8.29 (1H, d, J=8.76 Hz), 8.48 (1H, s), 9.16 (1H, dd, J=1.78, 0.96 Hz), 11.91 (1H, br s); ESIMS found for C₁₄H₁₁N₅O m/z 266.1 (M+1).

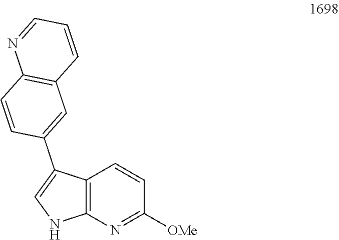 [SMILES: COc1ccc2c(-c3ccc4ncccc4c3)c[nH]c2n1]

### 6-(6-Methoxy-1H-pyrrolo[2,3-b]pyridin-3-yl)quinoline 1698

Beige solid (15.0 mg, 0.054 mmol, 12.4% yield). ¹H NMR (499 MHz, DMSO-d₆) δ ppm 3.91 (3H, s), 6.68 (1H, d, J=8.51 Hz), 7.52 (1H, dd, J=8.23, 4.12 Hz), 7.83 (1H, d, J=2.20 Hz), 8.02 (1H, d, J=8.78 Hz), 8.15 (1H, br d, J=8.78 Hz), 8.28 (1H, s), 8.38-8.49 (2H, m), 8.82 (1H, br d, J=3.02 Hz), 11.87 (1H, br s); ESIMS found for C₁₇H₁₃N₃O m/z 276.1 (M+1).

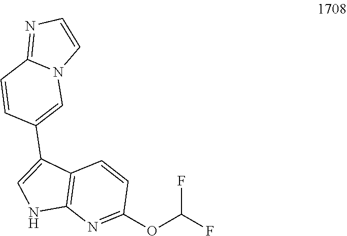 [SMILES: FC(F)Oc1ccc2c(-c3ccc4nccn4c3)c[nH]c2n1]

### 6-(6-(Difluoromethoxy)-1H-pyrrolo[2,3-b]pyridin-3-yl)imidazo[1,2-a] pyridine 1708

Off-white solid (65 mg, 0.217 mmol, 33.1% yield). ¹H NMR (499 MHz, DMSO-d₆) δ ppm 6.89 (1H, d, J=8.49 Hz), 7.70 (1H, t, J=73.50 Hz), 7.57 (1H, d, J=1.09 Hz), 7.61-7.64 (2H, m), 7.89 (1H, d, J=2.46 Hz), 7.98 (1H, d, J=0.82 Hz), 8.52 (1H, d, J=8.49 Hz), 8.95 (1H, t, J=1.37 Hz), 12.12 (1H, br s); ESIMS found for C₁₅H₁₀F₂N₄O m/z 301.1 (M+1).

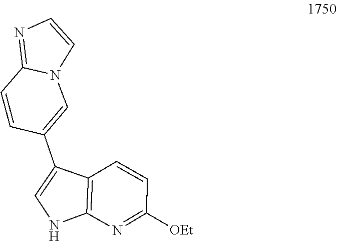 [SMILES: CCOc1ccc2c(-c3ccc4nccn4c3)c[nH]c2n1]

### 6-(6-Ethoxy-1H-pyrrolo[2,3-b]pyridin-3-yl)imidazo[1,2-a]pyridine 1750

Beige solid (30 mg, 0.108 mmol, 58.3% yield). ¹H NMR (499 MHz, DMSO-d₆) δ ppm 1.36 (3H, t, J=7.00 Hz), 4.35 (2H, q, J=6.95 Hz), 6.63 (1H, d, J=8.51 Hz), 7.55 (1H, d, J=1.10 Hz), 7.57-7.63 (2H, m), 7.68 (1H, d, J=2.74 Hz), 7.97 (1H, s), 8.30 (1H, d, J=8.51 Hz), 8.90 (1H, s), 11.75 (1H, br s); ESIMS found for C₁₆H₁₄N₄O m/z 279.1 (M+1).

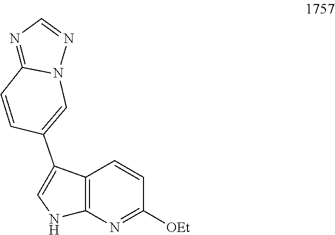 [SMILES: CCOc1ccc2c(-c3ccc4ncnn4c3)c[nH]c2n1]

### 6-(6-Ethoxy-1H-pyrrolo[2,3-b]pyridin-3-yl)-[1,2,4]triazolo[1,5-a] pyridine 1757

Beige solid (15 mg, 0.054 mmol, 15.5% yield). ¹H NMR (499 MHz, DMSO-d₆) δ ppm 1.36 (3H, t, J=6.98 Hz), 4.35 (2H, q, J=6.94 Hz), 6.62 (1H, d, J=8.49 Hz), 7.80 (1H, d, J=2.46 Hz), 7.89 (1H, d, J=9.03 Hz), 8.05 (1H, dd, J=9.17, 1.78 Hz), 8.28 (1H, d, J=8.49 Hz), 8.48 (1H, s), 9.11-9.20 (1H, m), 11.87 (1H, br s); ESIMS found for C₁₅H₁₃N₅O m/z 280.1 (M+1).

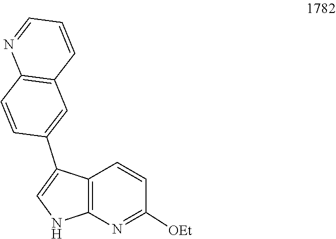 [SMILES: CCOc1ccc2c(-c3ccc4ncccc4c3)c[nH]c2n1]

### 6-(6-Ethoxy-1H-pyrrolo[2,3-b]pyridin-3-yl)quinoline 1782

Beige solid (20 mg, 0.069 mmol, 19.9% yield). ¹H NMR (499 MHz, DMSO-d₆) δ ppm 1.37 (3H, t, J=7.00 Hz), 4.36 (2H, q, J=7.04 Hz), 6.65 (1H, d, J=8.78 Hz), 7.51 (1H, dd, J=8.23, 4.12 Hz), 7.82 (1H, s), 8.01 (1H, d, J=8.78 Hz), 8.14 (1H, dd, J=8.92, 2.06 Hz), 8.27 (1H, d, J=1.92 Hz), 8.42 (2H, d, J=8.51 Hz), 8.82 (1H, dd, J=4.25, 1.78 Hz), 11.84 (1H, br s); ESIMS found for C₁₈H₁₅N₃O m/z 290.1 (M+1).

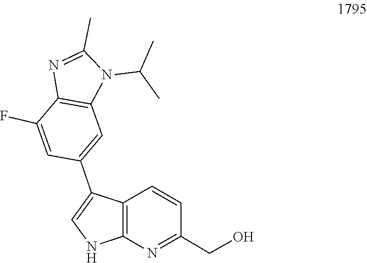 [SMILES: Cc1nc2c(F)cc(-c3c[nH]c4nc(CO)ccc34)cc2n1C(C)C]

### (3-(4-Fluoro-1-isopropyl-2-methyl-1H-benzo[d]imidazol-6-yl)-1H-pyrrolo[2,3-b]pyridin-6-yl)methanol 1795

Off-white solid (10 mg, 0.030 mmol, 9.7% yield). ¹H NMR (499 MHz, DMSO-d₆) δ ppm 1.61 (6H, d, J=6.86 Hz), 2.59 (3H, s), 4.63 (2H, d, J=5.49 Hz), 4.82 (1H, spt, J=6.86 Hz), 5.34 (1H, t, J=5.76 Hz), 7.26-7.33 (2H, m), 7.65 (1H, d, J=1.10 Hz), 7.88 (1H, d, J=1.37 Hz), 8.26 (1H, d, J=8.23 Hz), 11.78 (1H, br s); ESIMS found for C₁₉H₁₉FN₄O m/z 339.2 (M+1).

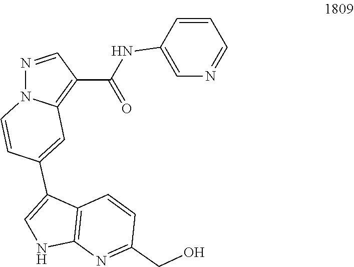 [SMILES: O=C(Nc1cccnc1)c1cnn2ccc(-c3c[nH]c4nc(CO)ccc34)cc12]

### 5-(6-(Hydroxymethyl)-1H-pyrrolo[2,3-b]pyridin-3-yl)-N-(pyridin-3-yl) pyrazolo[1,5-a]pyridine-3-carboxamide 1809

Beige solid (15 mg, 0.039 mmol, 12.4% yield). ¹H NMR (499 MHz, DMSO-d₆) δ ppm 4.66 (2H, d, J=5.48 Hz), 5.40 (1H, t, J=5.89 Hz), 7.38-7.42 (1H, m), 7.41 (1H, d, J=8.21 Hz), 7.57 (1H, dd, J=7.26, 2.05 Hz), 8.21-8.25 (2H, m), 8.30 (1H, dd, J=4.65, 1.37 Hz), 8.36 (1H, d, J=8.21 Hz), 8.58 (1H, d, J=1.37 Hz), 8.77 (1H, s), 8.81-8.87 (1H, m), 8.93 (1H, d, J=2.19 Hz), 10.13 (1H, s), 12.13 (1H, br s); ESIMS found for C₂₁H₁₆N₆O₂ m/z 385.05 (M+1).

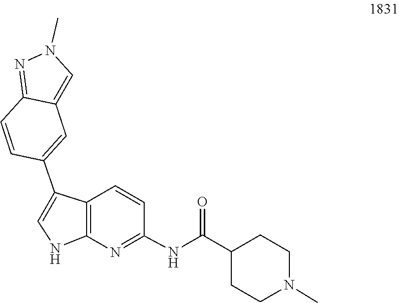 [SMILES: CN1CCC(C(=O)Nc2ccc3c(-c4ccc5nn(C)cc5c4)c[nH]c3n2)CC1]

### 1-Methyl-N-(3-(2-methyl-2H-indazol-5-yl)-1H-pyrrolo[2,3-b]pyridin-6-yl)piperidine-4-carboxamide 1831

White solid (17 mg, 0.044 mmol, 46.3% yield). ¹H NMR (499 MHz, DMSO-d₆) δ ppm 1.60-1.71 (2H, m), 1.72-1.79 (2H, m), 1.85 (2H, td, J=11.66, 2.20 Hz), 2.15 (3H, s), 2.42-2.49 (1H, m), 2.77-2.84 (2H, m), 4.17 (3H, s), 7.58-7.66 (2H, m), 7.71 (1H, d, J=1.65 Hz), 7.93-7.99 (2H, m), 8.28-8.33 (2H, m), 10.24 (1H, s), 11.56 (1H, s); ESIMS found for C₂₂H₂₄N₆O m/z 389.1 (M+1).

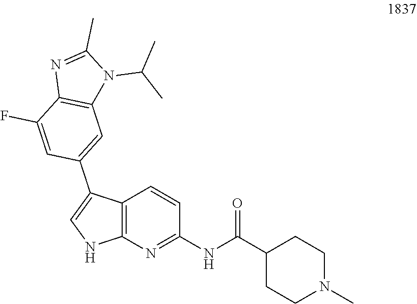 [SMILES: Cc1nc2c(F)cc(-c3c[nH]c4nc(NC(=O)C5CCN(C)CC5)ccc34)cc2n1C(C)C]

### N-(3-(4-Fluoro-1-isopropyl-2-methyl-1H-benzo[d]imidazol-6-yl)-1H-pyrrolo[2,3-b]pyridin-6-yl)-1-methylpiperidine-4-carboxamide 1837

White solid (38 mg, 0.085 mmol, 76.7% yield). ¹H NMR (499 MHz, DMSO-d₆) δ ppm 1.61 (6H, d, J=6.86 Hz), 1.63-1.71(2H, m), 1.72-1.79 (2H, m), 1.85 (2H, td, J=11.60, 2.06 Hz), 2.15 (3H, s), 2.59 (3H, s), 2.77-2.85 (2H, m), 4.82 (1H, spt, J=6.91 Hz), 7.28 (1H, dd, J=12.08, 1.10 Hz), 7.64 (1H, d, J=1.10 Hz), 7.80 (1H, d, J=1.92 Hz), 7.97 (1H, br d, J=8.78 Hz), 8.23 (1H, d, J=8.78 Hz), 10.24 (1H, s), 11.65 (1H, s); ESIMS found for C₂₅H₂₉FN₆O m/z 449.2 (M+1).

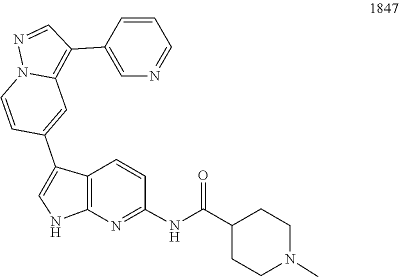 [SMILES: CN1CCC(C(=O)Nc2ccc3c(-c4ccn5ncc(-c6cccnc6)c5c4)c[nH]c3n2)CC1]

### 1-Methyl-N-(3-(3-(pyridin-3-yl)pyrazolo[1,5-a]pyridin-5-yl)-1H-pyrrolo[2,3-b]pyridin-6-yl)piperidine-4-carboxamide 1847

Beige solid (15 mg, 0.033 mmol, 21.8% yield). ¹H NMR (499 MHz, DMSO-d₆) δ ppm 1.60-1.71 (2H, m), 1.72-1.79 (2H, m), 1.86 (2H, td, J=11.64, 2.19 Hz), 2.16 (3H, s), 2.43-2.49 (1H, m), 2.77-2.85 (2H, m), 7.41 (1H, dd, J=7.39, 1.92 Hz), 7.49-7.54 (1H, m), 8.02 (1H, d, J=8.76 Hz), 8.08-8.13 (2H, m), 8.18 (1H, dt, J=8.21, 1.78 Hz), 8.35 (1H, d, J=8.76 Hz), 8.45 (1H, s), 8.48 (1H, dd, J=4.65, 1.64 Hz), 8.75-8.80 (1H, m), 9.01 (1H, dd, J=2.46, 0.82 Hz), 10.30 (1H, s), 11.90 (1H, br s); ESIMS found for C₂₆H₂₅N₇O m/z 452.1 (M+1).

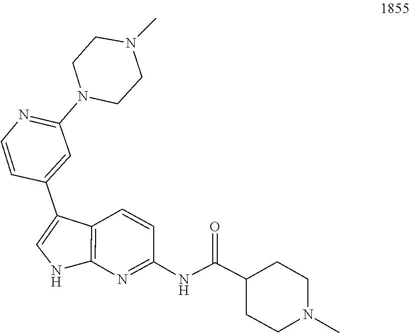 [SMILES: CN1CCC(C(=O)Nc2ccc3c(-c4ccnc(N5CCN(C)CC5)c4)c[nH]c3n2)CC1]

### 1-Methyl-N-(3-(2-(4-methylpiperazin-1-yl)pyridin-4-yl)-1H-pyrrolo [2,3-b]pyridin-6-yl)piperidine-4-carboxamide 1855

Off-white solid (25 mg, 0.058 mmol, 66.2% yield). ¹H NMR (499 MHz, DMSO-d₆) δ ppm 1.58-1.70 (2H, m), 1.71-1.79 (2H, m), 1.85 (2H, td, J=11.64, 2.19 Hz), 2.15 (3H, s), 2.23 (3H, s), 2.40-2.45 (4H, m), 2.45-2.48 (1H, m), 2.76-2.84 (2H, m), 3.50-3.59 (4H, m), 7.03 (1H, dd, J=5.20, 1.10 Hz), 7.04 (1H, s), 7.97 (1H, br d, J=8.76 Hz), 7.99 (1H, s), 8.10 (1H, d, J=5.48 Hz), 8.31 (1H, d, J=8.76 Hz), 10.26 (1H, s), 11.82 (1H, s); ESIMS found for C₂₄H₃₁N₇O m/z 434.15 (M+1).

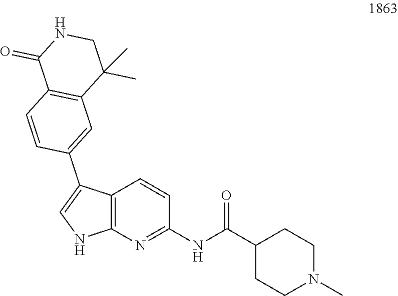 [SMILES: CN1CCC(C(=O)Nc2ccc3c(-c4ccc5c(c4)C(C)(C)CNC5=O)c[nH]c3n2)CC1]

### N-(3-(4,4-Dimethyl-1-oxo-1,2,3,4-tetrahydroisoquinolin-6-yl)-1H-pyrrolo[2,3-b]pyridin-6-yl)-1-methylpiperidine-4-carboxamide 1863

Off-white solid (8 mg, 0.019 mmol, 35.3% yield). ¹H NMR (499 MHz, DMSO-d₆) δ ppm 1.35 (6H, s), 1.60-1.70 (2H, m), 1.72-1.79 (2H, m), 1.82-1.90 (2H, m), 2.16 (3H, s), 2.77-2.84 (2H, m), 3.19 (2H, d, J=2.74 Hz), 7.67 (1H, s), 7.68 (1H, dd, J=7.80, 1.78 Hz), 7.86-7.91 (3H, m), 7.98 (1H, br d, J=8.21 Hz), 8.28 (1H, d, J=9.03 Hz), 10.26 (1H, s), 11.77 (1H, s); ESIMS found for C₂₅H₂₉N₅O₂ m/z 432.1 (M+1).

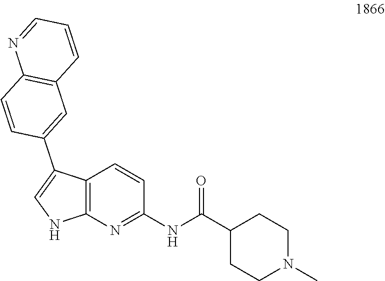 [SMILES: CN1CCC(C(=O)Nc2ccc3c(-c4ccc5ncccc5c4)c[nH]c3n2)CC1]

### 1-Methyl-N-(3-(quinolin-6-yl)-1H-pyrrolo[2,3-b]pyridin-6-yl) piperidine-4-carboxamide 1866

Yellow solid (25 mg, 0.065 mmol, 56.8% yield). ¹H NMR (500 MHz, DMSO-d₆) δ ppm 1.61-1.72 (2H, m), 1.73-1.80 (2H, m), 1.86 (2H, td, J=11.66, 2.20 Hz), 2.16 (3H, s), 2.45-2.49 (1H, m), 2.77-2.85 (2H, m), 7.52 (1H, dd, J=8.23, 4.12 Hz), 7.98 (1H, d, J=1.37 Hz), 8.01-8.05 (2H, m), 8.18 (1H, dd, J=8.78, 2.20 Hz), 8.31 (1H, d, J=1.92 Hz), 8.44 (1H, dd, J=8.65, 0.96 Hz), 8.50 (1H, d, J=8.78 Hz), 8.82 (1H, dd, J=4.12, 1.65 Hz), 10.29 (1H, s), 11.79 (1H, s); ESIMS found for C₂₃H₂₃N₅O m/z 386.2 (M+1).

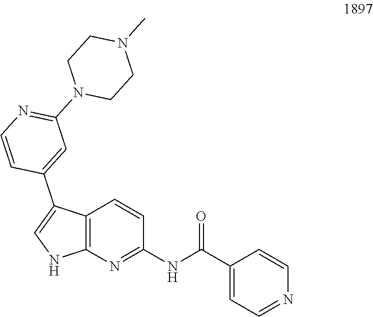 [SMILES: CN1CCN(c2cc(-c3c[nH]c4nc(NC(=O)c5ccncc5)ccc34)ccn2)CC1]

### N-(3-(2-(4-Methylpiperazin-1-yl)pyridin-4-yl)-1H-pyrrolo[2,3-b] pyridin-6-yl)isonicotinamide 1897

Off-white solid (8 mg, 0.019 mmol, 35.1% yield). ¹H NMR (499 MHz, DMSO-d₆) δ ppm 2.24 (3H, s), 2.41-2.47 (4H, m), 3.53-3.59 (4H, m), 7.05 (1H, dd, J=5.20, 1.37 Hz), 7.07 (1H, s), 7.91-7.95 (2H, m), 7.98 (1H, d, J=8.49 Hz), 8.06 (1H, s), 8.11 (1H, d, J=5.20 Hz), 8.39 (1H, d, J=8.76 Hz), 8.74-8.78 (2H, m), 11.42 (1H, br s); ESIMS found for C₂₃H₂₃N₇O m/z 414.1 (M+1).

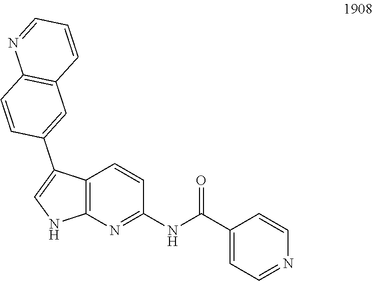 [SMILES: O=C(Nc1ccc2c(-c3ccc4ncccc4c3)c[nH]c2n1)c1ccncc1]

### N-(3-(Quinolin-6-yl)-1H-pyrrolo[2,3-b]pyridin-6-yl)isonicotinamide 1908

Beige solid (20 mg, 0.055 mmol, 44.6% yield). ¹H NMR (499 MHz, DMSO-d₆) δ ppm 7.54 (1H, dd, J=8.23, 4.12 Hz), 7.91-7.97 (2H, m), 8.05 (1H, d, J=1.65 Hz), 8.07 (2H, d, J=1.10 Hz), 8.21 (1H, dd, J=8.78, 1.92 Hz), 8.36 (1H, d, J=1.92 Hz), 8.45 (1H, d, J=7.41 Hz), 8.62 (1H, d, J=8.78 Hz), 8.75-8.80 (2H, m), 8.84 (1H, dd, J=4.12, 1.65 Hz), 11.02 (1H, s), 11.93 (1H, br s); ESIMS found for C₂₂H₁₅N₅O m/z 366.1 (M+1).

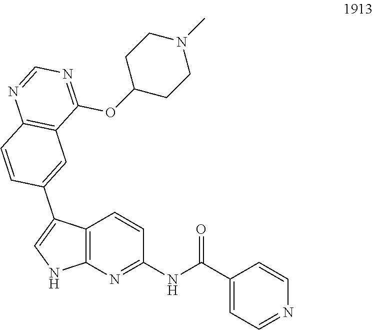 [SMILES: CN1CCC(Oc2ncnc3ccc(-c4c[nH]c5nc(NC(=O)c6ccncc6)ccc45)cc23)CC1]

### N-(3-(4-((1-Methylpiperidin-4-yl)oxy)quinazolin-6-yl)-1H-pyrrolo [2,3-b]pyridin-6-yl)isonicotinamide 1913

Beige solid (25 mg, 0.052 mmol, 30.3% yield). ¹H NMR (499 MHz, DMSO-d₆) δ ppm 1.91-1.98 (2H, m), 2.05-2.13 (2H, m), 2.24 (3H, s), 2.36 (2H, br dd, J=3.56, 1.92 Hz), 2.62-2.69 (2H, m), 5.40-5.48 (1H, m), 7.92-7.95 (2H, m), 7.96 (1H, d, J=8.76 Hz), 8.07 (1H, d, J=8.76 Hz), 8.09 (1H, d, J=2.19 Hz), 8.37 (1H, dd, J=8.76, 2.19 Hz), 8.39-8.43 (2H, m), 8.74 (1H, s), 8.77-8.80 (2H, m), 11.03 (1H, s), 12.00 (1H, br s); ESIMS found for C₂₇H₂₅N₇O₂ m/z 480.1 (M+1).

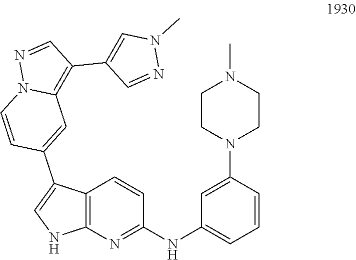 [SMILES: CN1CCN(c2cccc(Nc3ccc4c(-c5ccn6ncc(-c7cnn(C)c7)c6c5)c[nH]c4n3)c2)CC1]

### 3-(3-(1-Methyl-1H-pyrazol-4-yl)pyrazolo[1,5-a]pyridin-5-yl)-N-(3-(4-methylpiperazin-1-yl)phenyl)-1H-pyrrolo[2,3-b]pyridin-6-amine 1930

Beige solid (20 mg, 0.040 mmol, 32.0% yield). ¹H NMR (499 MHz, DMSO-d₆) δ ppm 2.24 (3H, s), 2.46-2.49 (4H, m), 3.11-3.19 (4H, m), 3.92 (3H, s), 6.48 (1H, dd, J=8.08, 2.05 Hz), 6.76 (1H, d, J=8.76 Hz), 7.09 (1H, t, J=8.08 Hz), 7.26 (2H, dd, J=7.39, 1.64 Hz), 7.47 (1H, t, J=2.05 Hz), 7.81 (1H, d, J=2.46 Hz), 7.88 (2H, s), 8.15-8.18 (3H, m), 8.60-8.68 (1H, m), 8.91 (1H, s), 11.65 (1H, s); ESIMS found for C₂₉H₂₉N₉ m/z 504.15 (M+1).

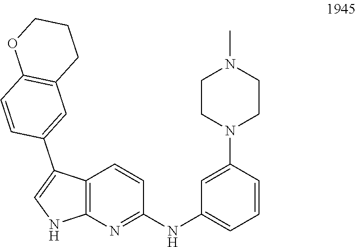 [SMILES: CN1CCN(c2cccc(Nc3ccc4c(-c5ccc6c(c5)CCCO6)c[nH]c4n3)c2)CC1]

### 3-(Chroman-6-yl)-N-(3-(4-methylpiperazin-1-yl)phenyl)-1H-pyrrolo [2,3-]pyridin-6-amine 1945

Brown solid (25 mg, 0.057 mmol, 16.5% yield). ¹H NMR (499 MHz, DMSO-d₆) δ ppm 1.90-2.00 (2H, m), 2.24 (3H, s), 2.45-2.49 (4H, m), 2.81 (2H, t, J=6.43 Hz), 3.09-3.18 (4H, m), 4.09-4.18 (2H, m), 6.46 (1H, dd, J=8.08, 1.78 Hz), 6.65 (1H, d, J=8.49 Hz), 6.72-6.79 (1H, m), 7.07 (1H, t, J=8.08 Hz), 7.21 (1H, dd, J=7.94, 1.10 Hz), 7.32 (1H, d, J=2.46 Hz), 7.32-7.35 (2H, m), 7.48 (1H, t, J=2.05 Hz), 8.00 (1H, d, J=8.49 Hz), 8.80 (1H, s), 11.26 (1H, d, J=1.64 Hz); ESIMS found for C₂₇H₂₉N₅O m/z 440.1 (M+1).

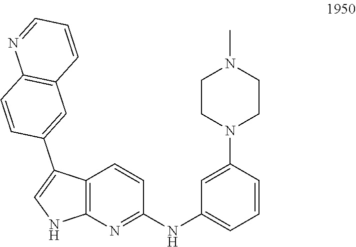 [SMILES: CN1CCN(c2cccc(Nc3ccc4c(-c5ccc6ncccc6c5)c[nH]c4n3)c2)CC1]

### N-(3-(4-Methylpiperazin-1-yl)phenyl)-3-(quinolin-6-yl)-1H-pyrrolo [2,3-b]pyridin-6-amine 1950

Beige solid (50 mg, 0.115 mmol, 52.9% yield). ¹H NMR (499 MHz, DMSO-d₆) δ ppm 2.24 (3H, s), 2.47-2.49 (4H, m), 3.11-3.20 (4H, m), 6.48 (1H, dd, J=8.10, 2.06 Hz), 6.75 (1H, d, J=8.78 Hz), 7.09 (1H, t, J=8.10 Hz), 7.23 (1H, dd, J=7.96, 1.37 Hz), 7.49-7.54 (2H, m), 7.74 (1H, d, J=2.47 Hz), 8.01 (1H, d, J=8.78 Hz), 8.15 (1H, dd, J=8.78, 1.92 Hz), 8.26 (1H, d, J=1.92 Hz), 8.32 (1H, d, J=8.51 Hz), 8.40-8.46 (1H, m), 8.81 (1H, dd, J=4.12, 1.65 Hz), 8.90 (1H, s), 11.58 (1H, d, J=1.92 Hz); ESIMS found for C₂₇H₂₆N₆ m/z 435.3 (M+1).

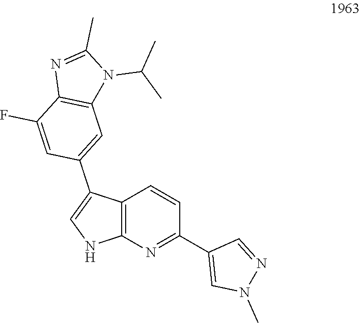 [SMILES: Cc1nc2c(F)cc(-c3c[nH]c4nc(-c5cnn(C)c5)ccc34)cc2n1C(C)C]

### 4-Fluoro-1-isopropyl-2-methyl-6-(6-(1-methyl-1H-pyrazol-4-yl)-1H-pyrrolo[2,3-b]pyridin-3-yl)-1H-benzo[d]imidazole 1963

Off-white solid (26.0 mg, 0.067 mmol, 43.1% yield). ¹H NMR (499 MHz, DMSO-d₆) δ ppm 1.62 (6H, d, J=6.86 Hz), 2.59 (3H, s), 3.91 (3H, s), 4.82 (1H, spt, J=6.91 Hz), 7.29 (1H, dd, J=12.08, 1.10 Hz), 7.48 (1H, d, J=8.51 Hz), 7.66 (1H, d, J=1.37 Hz), 7.86 (1H, d, J=2.47 Hz), 8.00 (1H, s), 8.22 (1H, d, J=8.51 Hz), 8.25 (1H, s), 11.84 (1H, s); ESIMS found for C₂₂H₂₁FN₆ m/z 389.2 (M+1).

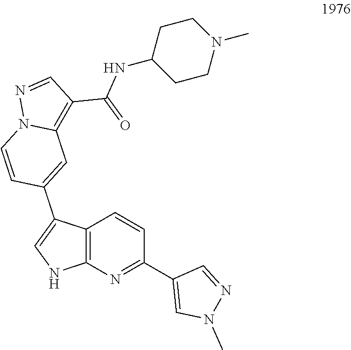 [SMILES: CN1CCC(NC(=O)c2cnn3ccc(-c4c[nH]c5nc(-c6cnn(C)c6)ccc45)cc23)CC1]

### 5-(6-(1-Methyl-1H-pyrazol-4-yl)-1H-pyrrolo[2,3-b]pyridin-3-yl)-N-(1-methylpiperidin-4-yl)pyrazolo[1,5-a]pyridine-3-carboxamide 1976

Brown solid (10 mg, 0.022 mmol, 32.7% yield). ¹H NMR (499 MHz, DMSO-d₆) δ ppm 1.53-1.66 (2H, m), 1.78-1.87 (2H, m), 1.97 (2H, br t, J=10.54 Hz), 2.18 (3H, s), 2.80 (2H, br d, J=11.50 Hz), 3.75-3.86 (1H, m), 3.92 (3H, s), 7.47 (1H, dd, J=7.39, 1.92 Hz), 7.57 (1H, d, J=8.49 Hz), 7.95 (1H, d, J=7.94 Hz), 8.01 (1H, s), 8.14 (1H, d, J=2.46 Hz), 8.27 (1H, s), 8.30 (1H, d, J=8.21 Hz), 8.56 (2H, s), 8.73 (1H, d, J=7.39 Hz), 12.12 (1H, br s) ESIMS found for C₂₅H₂₆N₈O m/z 455.1 (M+1).

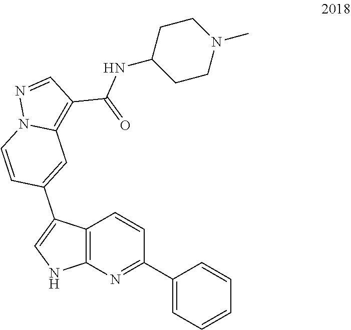 [SMILES: CN1CCC(NC(=O)c2cnn3ccc(-c4c[nH]c5nc(-c6ccccc6)ccc45)cc23)CC1]

### N-(1-Methylpiperidin-4-yl)-5-(6-phenyl-1H-pyrrolo[2,3-b]pyridin-3-yl)pyrazolo[1,5-a]pyridine-3-carboxamide 2018

Beige solid (10 mg, 0.022 mmol, 32.8% yield). ¹H NMR (499 MHz, DMSO-d₆) δ ppm 1.59 (2H, qd, J=11.96, 3.83 Hz), 1.77-1.87 (2H, m), 1.93-2.02 (2H, m), 2.18 (3H, s), 2.80 (2H, br d, J=11.50 Hz), 3.74-3.88 (1H, m), 7.39-7.46 (1H, m), 7.48-7.55 (3H, m), 7.91 (1H, d, J=8.49 Hz), 7.97 (1H, d, J=7.94 Hz), 8.13-8.19 (2H, m), 8.27 (1H, s), 8.43 (1H, d, J=8.49 Hz), 8.57 (1H, s), 8.61 (1H, d, J=1.64 Hz), 8.75 (1H, d, J=7.39 Hz), 12.30 (1H, br s); ESIMS found for C₂₇H₂₆N₆O m/z 451.25 (M+1).

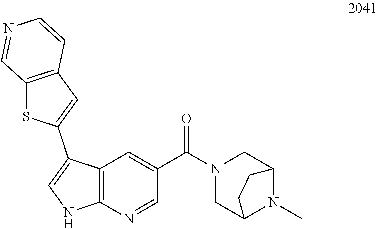 [SMILES: CN1C2CCC1CN(C(=O)c1cnc3[nH]cc(-c4cc5ccncc5s4)c3c1)C2]

### (8-Methyl-3,8-diazabicyclo[3.2.1]octan-3-yl)(3-(thieno[2,3-c]pyridin-2-yl)-1H-pyrrolo[2,3-b]pyridin-5-yl)methanone 2041

Beige solid (8 mg, 0.020 mmol, 8.4% yield). ¹H NMR (499 MHz, DMSO-d₆) δ ppm 1.34-1.49 (1H, m), 1.54-1.69 (1H, m), 1.81-1.98 (3H, m), 2.18(3H, s), 2.93-3.05 (2H, m), 3.11-3.22 (1H, m), 3.42-3.56 (1H, m), 4.20-4.36 (1H, m), 7.77 (1H, dd, J=5.48, 1.10 Hz), 7.88 (1H, s), 8.27 (1H, s), 8.37 (1H, d, J=1.92 Hz), 8.44 (1H, d, J=2.46 Hz), 8.44 (1H, d, J=0.82 Hz), 9.16 (1H, s), 12.55 (1H, br s); ESIMS found for C₂₂H₂₁N₅OS m/z 404.1 (M+1).

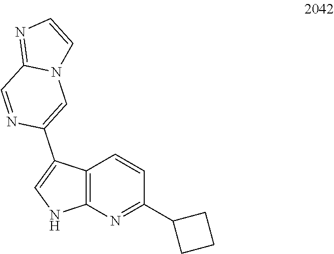 [SMILES: c1cn2cc(-c3c[nH]c4nc(C5CCC5)ccc34)ncc2n1]

### 6-(6-Cyclobutyl-1H-pyrrolo[2,3-b]pyridin-3-yl)imidazo[1,2-a]pyrazine 2042

Beige solid (15 mg, 0.052 mmol, 44.5% yield). ¹H NMR (499 MHz, DMSO-d₆) δ ppm 1.83-1.93 (1H, m), 1.96-2.10 (1H, m), 2.28-2.40 (4H, m), 3.75 (1H, quin, J=8.69 Hz), 7.08 (1H, d, J=8.21 Hz), 7.80 (1H, d, J=0.82 Hz), 8.00 (1H, d, J=2.74 Hz), 8.12 (1H, s), 8.48 (1H, d, J=7.94 Hz), 9.05 (1H, d, J=1.37 Hz), 9.12 (1H, d, J=0.82 Hz), 11.92 (1H, br s); ESIMS found for C₁₇H₁₅N₅ m/z 290.1 (M+1).

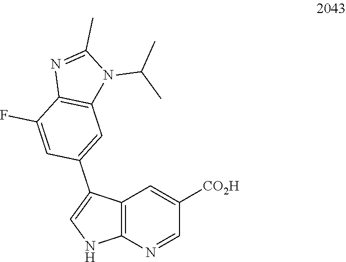 [SMILES: Cc1nc2c(F)cc(-c3c[nH]c4ncc(C(=O)O)cc34)cc2n1C(C)C]

### 3-(4-Fluoro-1-isopropyl-2-methyl-1H-benzo[d]imidazol-6-yl)-1H-pyrrolo[2,3-b]pyridine-5-carboxylic acid 2043

Off-white solid (140 mg, 0.397 mmol, 88.0% yield). ¹H NMR (499 MHz, DMSO-d₆) δ ppm 1.62 (6H, d, J=6.86 Hz), 2.60 (3H, s), 4.81 (1H, spt, J=6.93 Hz), 7.30 (1H, dd, J=11.94, 0.96 Hz), 7.70 (1H, d, J=1.10 Hz), 8.06 (1H, d, J=2.47 Hz), 8.74 (1H, d, J=1.65 Hz), 8.85 (1H, d, J=1.92 Hz), 12.32 (1H, br s); ESIMS found for C₁₉H₁₇FN₄O₂ m/z 353.1 (M+1).

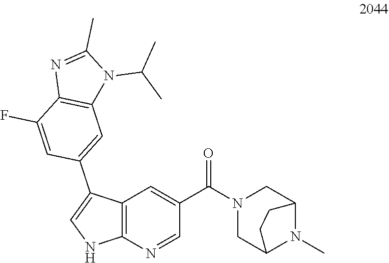 [SMILES: Cc1nc2c(F)cc(-c3c[nH]c4ncc(C(=O)N5CC6CCCC(C5)N6C)cc34)cc2n1C(C)C]

### (3-(4-Fluoro-1-isopropyl-2-methyl-1H-benzo[d]imidazol-6-yl)-1H-pyrrolo[2,3-b]pyridin-5-yl)(8-methyl-3,8-diazabicyclo[3.2.1]octan-3-yl)methanone 2044

White solid (70.0 mg, 0.015 mmol, 12.5% yield). ¹HNMR (499 MHz, DMSO-d₆) δ ppm 1.37-1.51 (1H, m), 1.61 (6H, d, J=6.86 Hz), 1.88 (2H, br s), 2.16 (3H, s), 2.59 (3H, s), 2.89-3.07 (2H, m), 3.08-3.20 (1H, m), 3.34-3.46 (3H, m), 4.14-4.34 (1H, m), 4.81 (1H, spt, J=6.86 Hz), 7.25-7.31 (1H, m), 7.66 (1H, d, J=1.10 Hz), 8.03 (1H, s), 8.20 (1H, d, J=1.92 Hz), 8.30 (1H, d, J=1.92 Hz), 12.19 (1H, br s); ESIMS found for C₂₆H₂₉FN₆O m/z 461.2 (M+1).

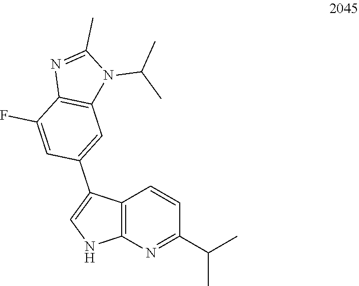 [SMILES: Cc1nc2c(F)cc(-c3c[nH]c4nc(C(C)C)ccc34)cc2n1C(C)C]

### 4-Fluoro-1-isopropyl-6-(6-isopropyl-1H-pyrrolo[2,3-b]pyridin-3-yl)-2-methyl-1H-benzo[d]imidazole 2045

White solid (30 mg, 0.086 mmol, 28.% yield). ¹H NMR (500 MHz, DMSO-d₆) δ ppm 1.30 (6H, d, J=6.86 Hz), 1.61 (6H, d, J=6.86 Hz), 1.70-1.81 (1H, m), 2.59 (3H, s), 4.75-4.86 (1H, m), 7.04-7.13 (1H, m), 7.24-7.31 (1H, m), 7.64 (1H, s), 7.83 (1H, t, J=2.20 Hz), 8.13-8.20 (1H, m), 11.75 (1H, br s); ESIMS found for C₂₁H₂₃FN₄ m/z 351.2 (M+1).

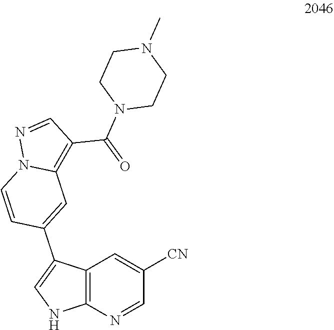 [SMILES: CN1CCN(C(=O)c2cnn3ccc(-c4c[nH]c5ncc(C#N)cc45)cc23)CC1]

### 3-(3-(4-Methylpiperazine-1-carbonyl)pyrazolo[1,5-a]pyridin-5-yl)-1H-pyrrolo[2,3-b]pyridine-5-carbonitrile 2046

Light yellow solid (42 mg, 0.109 mmol, 46.1% yield). ¹H NMR (499 MHz, DMSO-d₆) δ ppm 2.22 (3H, s), 2.39 (4H, t, J=4.94 Hz), 3.64-3.71 (4H, m), 7.51 (1H, dd, J=7.27, 2.06 Hz), 8.13 (1H, d, J=1.37 Hz), 8.26 (1H, s), 8.42 (1H, s), 8.72 (1H, d, J=1.92 Hz), 8.80-8.83 (2H, m), 12.84 (1H, br s); ESIMS found for C₂₁H₁₉N₇O m/z 386.1 (M+1).

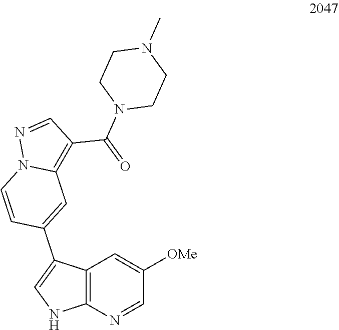 [SMILES: COc1cnc2[nH]cc(-c3ccn4ncc(C(=O)N5CCN(C)CC5)c4c3)c2c1]

### (5-(5-Methoxy-1H-pyrrolo[2,3-b]pyridin-3-yl)pyrazolo[1,5-a]pyridin-3-yl)(4-methylpiperazin-1-yl)methanone 2047

Light yellow solid (5 mg, 0.013 mmol, 5.9% yield). ¹H NMR (499 MHz, DMSO-d₆) δ ppm 2.21 (3H, s), 2.37 (4H, br t, J=4.94 Hz), 3.65-3.72 (4H, m), 3.91 (3H, s), 7.48 (1H, dd, J=7.14, 1.92 Hz), 7.82 (1H, d, J=2.74 Hz), 8.10 (1H, d, J=1.65 Hz), 8.12 (1H, d, J=2.47 Hz), 8.17 (1H, d, J=2.47 Hz), 8.23 (1H, s), 8.77 (1H, d, J=7.14 Hz), 12.09 (1H, br s); ESIMS found for C₂₁H₂₂N₆O₂ m/z 391.1 (M+1).

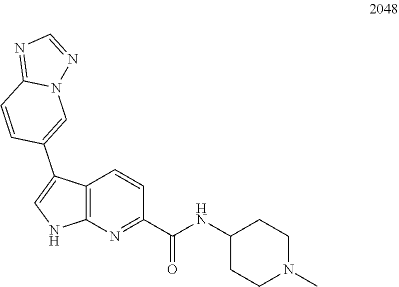 [SMILES: CN1CCC(NC(=O)c2ccc3c(-c4ccc5ncnn5c4)c[nH]c3n2)CC1]

### 3-([1,2,4]Triazolo[1,5-a]pyridin-6-yl)-N-(1-methylpiperidin-4-yl)-1H-pyrrolo[2,3-b]pyridine-6-carboxamide 2048

Beige solid (10 mg, 0.027 mmol, 18.0% yield). ¹H NMR (499 MHz, DMSO-d₆) δ ppm 1.59-1.71 (2H, m), 1.78-1.87 (2H, m), 2.02 (2H, br t, J=10.81 Hz), 2.18 (3H, s), 2.75 (2H, br d, J=11.77 Hz), 3.73-3.85 (1H, m), 7.90 (1H, d, J=8.21 Hz), 7.94 (1H, d, J=9.31 Hz), 8.12 (1H, dd, J=9.31, 1.92 Hz), 8.19 (1H, d, J=8.21 Hz), 8.28 (1H, s), 8.52 (1H, s), 8.55 (1H, d, J=8.21 Hz), 9.26-9.31 (1H, m), 12.29 (1H, br s); ESIMS found for C₂₀H₂₁N₇O m/z 376.2 (M+1).

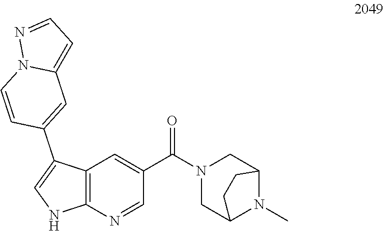 [SMILES: CN1C2CCC1CN(C(=O)c1cnc3[nH]cc(-c4ccn5nccc5c4)c3c1)C2]

### (8-Methyl-3,8-diazabicyclo[3.2.1]octan-3-yl)(3-(pyrazolo[1,5-a] pyridin-5-yl)-1H-pyrrolo[2,3-b]pyridin-5-yl)methanone 2049

White solid (4 mg, 0.010 mmol, 9.2% yield). ¹H NMR (499 MHz, DMSO-d₆) δ ppm 1.34-1.51 (1H, m), 1.54-1.70 (1H, m), 1.83-1.95 (2H, m), 2.18(3H, s), 2.89-3.06 (2H, m), 3.11-3.23 (2H, m), 4.20-4.35 (1H, m), 6.58-6.66 (1H, m), 7.30 (1H, dd, J=7.14, 1.92 Hz), 7.97 (1H, d, J=2.20 Hz), 8.04 (1H, d, J=1.10 Hz), 8.21 (1H, d, J=2.20 Hz), 8.32 (1H, d, J=1.92 Hz), 8.44 (1H, d, J=1.92 Hz), 8.68 (1H, d, J=7.14 Hz), 12.33 (1H, br s); ESIMS found for C₂₂H₂₂N₆O m/z 387.1 (M+1).

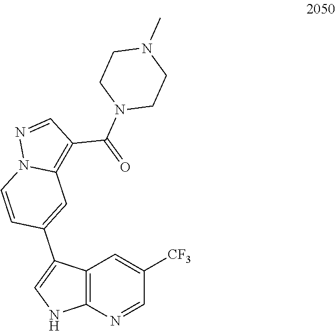 [SMILES: CN1CCN(C(=O)c2cnn3ccc(-c4c[nH]c5ncc(C(F)(F)F)cc45)cc23)CC1]

### (4-Methylpiperazin-1-yl)(5-(5-(trifluoromethyl)-1H-pyrrolo[2,3-b] pyridin-3-yl)pyrazolo[1,5-a]pyridin-3-yl)methanone 2050

White solid (19.0 mg, 0.044 mmol, 37.7% yield). ¹H NMR (499 MHz, DMSO-d₆) δ ppm 2.22 (3H, s), 2.38 (4H, t, J=5.08 Hz), 3.65-3.72 (4H, m), 7.48 (1H, dd, J=7.27, 2.06 Hz), 8.11 (1H, d, J=1.37 Hz), 8.14 (1H, dd, J=9.88, 2.74 Hz), 8.24 (1H, s), 8.33 (1H, s), 8.34 (1H, dd, J=2.47, 1.37 Hz), 8.78 (1H, d, J=7.41 Hz), 12.39 (1H, br s); ESIMS found for C₂₁H₁₉F₃N₆O m/z 429.0 (M+1).

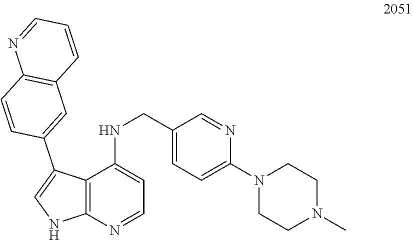 [SMILES: CN1CCN(c2ccc(CNc3ccnc4[nH]cc(-c5ccc6ncccc6c5)c34)cn2)CC1]

### N-((6-(4-Methylpiperazin-1-yl)pyridin-3-yl)methyl)-3-(quinolin-6-yl)-1H-pyrrolo[2,3-b]pyridin-4-amine 2051

Beige solid (10 mg, 0.022 mmol, 9.7% yield). ¹H NMR (499 MHz, DMSO-d₆) δ ppm 2.20 (3H, s), 2.36 (4H, t, J=4.94 Hz), 3.38-3.43 (4H, m), 4.23 (2H, d, J=5.49 Hz), 5.16 (1H, t, J=5.35 Hz), 6.31 (1H, d, J=5.49 Hz), 6.73 (1H, d, J=8.51 Hz), 7.35 (1H, d, J=1.65 Hz), 7.50-7.55 (2H, m), 7.89(1 dd, J=8.51, 1.92 Hz), 7.92 (1H, d, J=5.21 Hz), 7.97 (1 d, J=1.92 Hz), 8.01 (1H, d, J=8.51 Hz), 8.09 (1H, d, J=2.20 Hz), 8.28 (1H, d, J=7.68 Hz), 8.85 (1H, dd, J=4.25, 1.78 Hz), 11.61 (1H, s); ESIMS found for C₂₇H₂₇N₇ m/z 450.25 (M+1).

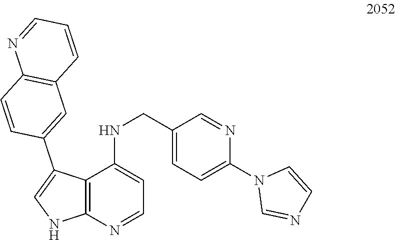 [SMILES: c1cnc2ccc(-c3c[nH]c4nccc(NCc5ccc(-n6ccnc6)nc5)c34)cc2c1]

### N-((6-(1H-Imidazol-1-yl)pyridin-3-yl)methyl)-3-(quinolin-6-yl)-1H-pyrrolo[2,3-b]pyridin-4-amine 2052

Beige solid (10 mg, 0.024 mmol, 10.4% yield). ¹H NMR (499 MHz, DMSO-d₆) δ ppm 4.46 (2H, d, J=5.49 Hz), 5.50 (1H, t, J=5.76 Hz), 6.28 (1H, d, J=5.49 Hz), 7.12 (1H, s), 7.37 (1H, s), 7.49 (1H, dd, J=8.23, 4.12 Hz), 7.74 (1H, d, J=8.23 Hz), 7.90-7.95 (3H, m), 7.98 (1H, dd, J=8.51, 2.20 Hz), 8.02 (1H, d, J=1.65 Hz), 8.04 (1H, d, J=8.78 Hz), 8.32 (1H, d, J=7.41 Hz), 8.48 (1H, s), 8.49 (1H, d, J=1.92 Hz), 8.83 (1H, dd, J=4.12, 1.65 Hz), 11.64 (1H, s); ESIMS found for C₂₅H₁₉N₇ m/z 418.15 (M+1).

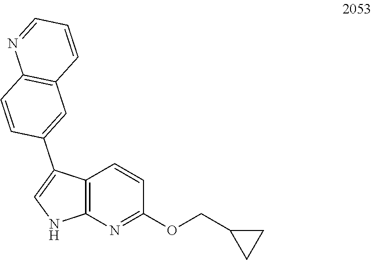 [SMILES: c1cnc2ccc(-c3c[nH]c4nc(OCC5CC5)ccc34)cc2c1]

### 6-(6-(Cyclopropylmethoxy)-1H-pyrrolo[2,3-b]pyridin-3-yl)quinoline 2053

Beige solid (20 mg, 0.063 mmol, 20.8% yield). ¹H NMR (499 MHz, DMSO-d₆) δ ppm 0.32-0.40 (2H, m), 0.55-0.62 (2H, m), 1.24-1.36 (1H, m), 4.14 (2H, d, J=6.86 Hz), 6.68 (1H, d, J=8.51 Hz), 7.51 (1H, dd, J=8.23, 4.39 Hz), 7.82 (1H, s), 8.01 (1H, d, J=8.78 Hz), 8.14 (1H, dd, J=8.92, 2.06 Hz), 8.28 (1H, d, J=1.92 Hz), 8.39-8.47 (2H, m), 8.82 (1H, dd, J=4.12, 1.65 Hz), 11.81 (1H, br s); ESIMS found for C₂₀H₁₇N₃O m/z 316.1 (M+1).

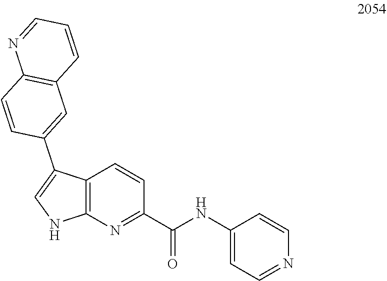 [SMILES: O=C(Nc1ccncc1)c1ccc2c(-c3ccc4ncccc4c3)c[nH]c2n1]

### N-(Pyridin-4-yl)-3-(quinolin-6-yl)-1H-pyrrolo[2,3-b]pyridine-6-carboxamide 2054

Beige solid (15 mg, 0.041 mmol, 17.4% yield). ¹H NMR (499 MHz, DMSO-d₆) δ ppm 7.51-7.62 (1H, m), 7.93 (2H, br s), 8.07 (2H, br dd, J=19.76, 7.96 Hz), 8.24 (1H, br d, J=8.78 Hz), 8.36-8.43 (2H, m), 8.47 (1H, br d, J=7.68 Hz), 8.52 (2H, br s), 8.78 (1H, br d, J=7.96 Hz), 8.86 (1H, br s), 10.77 (1H, br s), 12.37 (1H, br s); ESIMS found for C₂₂H₁₅N₅O m/z 366.05 (M+1).

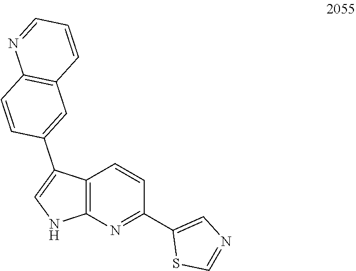 [SMILES: c1cnc2ccc(-c3c[nH]c4nc(-c5cncs5)ccc34)cc2c1]

### 5-(3-(quinolin-6-yl)-1H-pyrrolo[2,3-b]pyridin-6-yl)thiazole 2055

Beige solid (5 mg, 0.015 mmol, 47.6% yield). ¹H NMR (499 MHz, DMSO-d₆) δ ppm 7.51-7.62 (1H, m), 7.93 (2H, br s), 8.07 (2H, br dd, J=19.76, 7.96 Hz), 8.24 (1H, br d, J=8.78 Hz), 8.36-8.43 (2H, m), 8.47 (1H, br d, J=7.68 Hz), 8.52 (2H, br s), 8.78 (1H, br d, J=7.96 Hz), 8.86 (1H, br s), 10.77 (1H, br s), 12.37 (1H, br s); ESIMS found for C₁₉H₁₂N₄S m/z 329.0 (M+1).

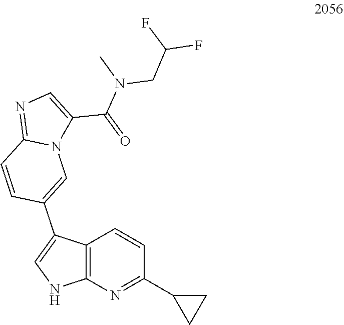 [SMILES: CN(CC(F)F)C(=O)c1cnc2ccc(-c3c[nH]c4nc(C5CC5)ccc34)cn12]

### 6-(6-cyclopropyl-1H-pyrrolo[2,3-b]pyridin-3-yl)-N-(2,2-difluoroethyl)-N-methylimidazo[1,2-a]pyridine-3-carboxamide 2056

Off-white solid (14 mg, 0.035 mmol, 29.5% yield). ¹H NMR (499 MHz, DMSO-d₆) δ ppm 0.92-1.01 (4H, m), 2.13-2.24 (1H, m), 3.38 (3H, br s), 4.03 (2H, td, J=15.33, 3.83 Hz), 6.35 (1H, tt, J=55.80, 3.90 Hz), 7.18 (1H, d, J=8.21 Hz), 7.79 (1H, d, J=9.03 Hz), 7.86-7.89 (1H, m), 7.89 (1H, d, J=2.74 Hz), 8.10 (1H, d, J=8.21 Hz), 8.17 (1H, s), 9.43 (1H, br s), 11.86 (1H, br d, J=1.37 Hz); ESIMS found for C₂₁H₁₉F₂N₅O m/z 396.2 (M+1).

### Example 9

Representative compounds were screened using the assay procedure for DYRK1A kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, Calif.) into 1536-well black-walled round bottom plates (Corning).

The DYRK1A kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant DYRK1A kinase, ATP and Ser/Thr peptide 18 were prepared in 1× Kinase buffer to final concentrations of 0.25 μg/mL, 15 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 uM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio×F100%)−C100%)/((C0%−C100%)+(Em ratio×(F100%−F0%)))]. Dose-response curves were generated, and inhibitory concentration (IC₅₀) values were calculated using non-linear regression curve fit in the Dotmatics' Studies Software (Bishops Stortford, UK).

Table 2 shows the measured activity for representative compounds of Formula I as described herein.

TABLE 2

| Compound | EC₅₀ (μM) |
|---|---|
| 2 | 0.050 |
| 3 | 0.006 |
| 4 | 0.007 |
| 5 | 0.010 |
| 7 | 0.201 |
| 8 | 0.007 |
| 9 | 0.019 |
| 10 | 0.014 |
| 11 | 0.012 |
| 12 | 0.003 |
| 13 | 0.021 |
| 14 | 0.001 |
| 18 | 0.017 |
| 20 | 0.010 |
| 21 | 0.016 |
| 23 | 0.002 |
| 24 | 0.001 |
| 25 | 0.001 |
| 26 | 0.058 |
| 27 | 0.002 |
| 28 | 0.019 |
| 29 | 0.010 |
| 30 | 0.013 |
| 31 | 0.004 |
| 32 | 0.151 |
| 34 | 0.002 |
| 35 | 0.002 |
| 36 | 0.002 |
| 37 | 0.001 |
| 38 | 0.002 |
| 41 | 0.002 |
| 42 | 0.002 |
| 43 | 0.001 |
| 44 | 0.001 |
| 45 | 0.001 |
| 46 | 0.005 |
| 47 | 0.002 |
| 48 | 0.001 |
| 49 | 0.001 |
| 50 | 0.001 |
| 51 | 0.001 |
| 52 | 0.002 |
| 53 | 0.007 |
| 54 | 0.018 |
| 55 | 0.004 |
| 56 | 0.007 |
| 57 | 0.003 |
| 58 | 0.012 |
| 59 | 0.081 |
| 60 | 0.006 |
| 61 | 0.004 |
| 62 | 0.005 |
| 63 | 0.005 |
| 64 | 0.006 |
| 65 | 0.007 |
| 67 | 0.019 |
| 69 | 0.073 |
| 70 | 0.013 |
| 72 | 0.091 |
| 76 | 0.031 |
| 78 | 0.006 |
| 79 | 0.024 |
| 80 | 0.031 |
| 82 | 0.006 |
| 83 | 0.029 |
| 84 | 0.003 |
| 85 | 0.025 |
| 86 | 0.007 |
| 87 | 0.002 |
| 88 | 0.003 |
| 89 | 0.001 |
| 90 | 0.003 |
| 91 | 0.003 |
| 92 | 0.005 |
| 93 | 0.004 |
| 95 | 0.002 |
| 96 | 0.001 |
| 97 | 0.002 |
| 98 | 0.002 |
| 99 | 0.002 |
| 100 | 0.006 |
| 101 | 0.006 |
| 102 | 0.002 |
| 103 | 0.001 |
| 104 | 0.011 |
| 105 | 0.004 |
| 106 | 0.003 |
| 107 | 0.008 |
| 108 | 0.052 |
| 128 | 0.001 |
| 186 | 0.010 |
| 215 | 0.012 |
| 226 | 0.036 |
| 257 | 0.016 |
| 280 | 0.004 |
| 325 | 0.002 |
| 367 | 0.002 |
| 409 | 0.013 |
| 435 | 0.096 |
| 439 | 0.016 |
| 451 | 0.004 |
| 482 | 0.015 |
| 493 | 0.007 |
| 502 | 0.016 |
| 521 | 0.009 |
| 535 | 0.002 |
| 573 | 0.010 |
| 577 | 0.003 |
| 619 | 0.000 |
| 648 | 0.002 |
| 661 | 0.002 |
| 703 | 0.003 |
| 764 | 0.022 |
| 787 | 0.003 |
| 792 | 0.010 |
| 802 | 0.007 |
| 813 | 0.003 |
| 817 | 0.008 |
| 829 | 0.002 |
| 871 | 0.002 |
| 913 | 0.004 |
| 955 | 0.020 |
| 997 | 0.006 |
| 1036 | 0.002 |
| 1055 | 0.009 |
| 1074 | 0.019 |
| 1081 | 0.002 |
| 1123 | 0.004 |
| 1137 | 0.004 |
| 1154 | 0.010 |
| 1166 | 0.003 |
| 1178 | 0.006 |
| 1192 | 0.010 |
| 1194 | 0.000 |
| 1197 | 0.003 |
| 1198 | 0.003 |
| 1207 | 0.002 |
| 1249 | 0.002 |
| 1291 | 0.007 |
| 1333 | 0.001 |
| 1362 | 0.001 |
| 1370 | 0.006 |
| 1372 | 0.001 |
| 1375 | 0.002 |
| 1384 | 0.001 |
| 1398 | 0.004 |
| 1404 | 0.001 |
| 1446 | 0.011 |
| 1459 | 0.004 |
| 1481 | 0.057 |
| 1488 | 0.002 |
| 1495 | 0.105 |
| 1498 | 0.047 |
| 1499 | 0.001 |
| 1500 | 0.005 |
| 1502 | 0.005 |
| 1503 | 0.007 |
| 1505 | 0.100 |
| 1507 | 0.038 |
| 1508 | 0.029 |
| 1513 | 0.091 |
| 1514 | 0.005 |
| 1517 | 0.066 |
| 1522 | 0.012 |
| 1526 | 0.010 |
| 1529 | 0.028 |
| 1530 | 0.024 |
| 1533 | 0.229 |
| 1534 | 0.012 |
| 1543 | 0.043 |
| 1546 | 2.053 |
| 1547 | 0.121 |
| 1554 | 0.008 |
| 1556 | 0.005 |
| 1558 | 0.291 |
| 1571 | 0.050 |
| 1577 | 0.214 |
| 1598 | 0.004 |
| 1640 | 0.006 |
| 1666 | 0.018 |
| 1673 | 0.065 |
| 1698 | 0.007 |
| 1708 | 0.018 |
| 1750 | 0.009 |
| 1757 | 0.144 |
| 1782 | 0.015 |
| 1795 | 0.008 |
| 1809 | 0.003 |
| 1831 | 0.001 |
| 1837 | 0.001 |
| 1847 | 0.001 |
| 1855 | 0.030 |
| 1863 | 0.005 |
| 1866 | 0.001 |
| 1897 | 0.051 |
| 1898 | 0.020 |
| 1899 | 0.014 |
| 1908 | 0.001 |
| 1913 | 0.005 |
| 1930 | 0.004 |
| 1945 | 0.125 |
| 1950 | 0.038 |
| 1963 | 0.045 |
| 1976 | 0.010 |
| 2018 | 0.007 |
| 2041 | 0.115 |
| 2042 | 0.345 |
| 2043 | 0.036 |
| 2044 | 0.003 |
| 2045 | 0.036 |
| 2046 | 0.047 |
| 2047 | 0.032 |
| 2048 | 2.543 |
| 2049 | 0.045 |
| 2050 | 0.159 |
| 2051 | 0.037 |
| 2052 | 0.034 |
| 2053 | 0.076 |
| 2054 | 0.051 |
| 2055 | 0.037 |
| 2056 | 0.005 |

### Example 10

Representative compounds were screened using the assay procedure for tau phosphorylation activity described below.

HEK293T cells (ATCC, CRL3216) cultured in DMEM (Thermo Fisher Scientific, 10566024) supplemented with 10% FBS (Corning, 35-011-CV) and Penicillin/Streptomycin (Thermo Fisher Scientific, 15140163) were seeded in a 75 cm² flask at 8.1×10⁶ cells/flask. The HEK293T cells were then transiently transfected with 5 μg DYRK1A (NM_001396) human untagged clone (OriGene, SC314641) and 2.5 μg MAPT (441 a.a. Tau gene) (NM_005910) human untagged clone (OriGene, TP313312) using Lipofectamine 3000 (Thermo Fisher Scientific, L30000015) and incubated for 20-30 hours in a humidified incubator at 37° C. and 5% CO₂. Post-incubation, HEK293T cells transfected with the DYRK1A and MAPT expression vectors were harvested and seeded in BioCoat poly-D lysine coated 96-well plates (Corning, 354461) at 3×10⁴ cells/well.

The above synthesized compounds were screened using the cell assay procedure to assess decreased Tau phosphorylation at Thr212 (pThr212) described below.

Each compound was dissolved in DMSO (Sigma-Aldrich, D8418-100 mL) as a 10 mM stock. 10 mM stocks were serially diluted 1:3, 10-point dose-response curve and added to the cells with a final concentration ranging from 20 μM to 1.1 nM. Cell were treated with compounds in duplicate and incubated for 18-24 hours in a humidified incubator at 37° C. and 5% CO₂.

Following the overnight compound treatment, cells were lysed with 1× Alpha Surefire Ultra Lysis Buffer (Perkin Elmer, ALSU-LB-100ML) complemented with 1× Halt Phosphatase Inhibitor Cocktail (Thermo Fisher Scientific, 78427) and 1× Halt Protease Inhibitor Cocktail (Thermo Fisher Scientific, 78438). Lysates were spun down at 12,000 g for 10 min to remove any cellular debris and 5 μL of lysates were dispensed into a 384-well Opti-Plate (Perkin Elmer, 6007290) for the measurement of Tau phosphorylation in the phosphoTau (Thr212) AlphaLISA assay. Donor antibody, biotinylated HT7Tau (Thermo Fisher Scientific, MN1000B), and acceptor antibody, pThr212Tau (Thermo Fisher Scientific, 44740G) were both added to the cell lysates at a final concentration of 3 nM and incubated for 1 hour at room temperature. Following incubation of the lysates with the donor and acceptor antibodies, anti-rabbit IgG(Fc specific) AlphaLISA acceptor beads (Perkin Elmer, AL104C) were added at a 10 ug/mL final concentration and incubated for 1 hour at room temperature protected from light. Lastly, AlphaScreen streptavidin donor beads (PerkinElmer, 6760002) were added at 40 ug/mL final concentration and incubated for 1 hour at room temperature protected from light. Plates were read at Ex=665 nm, and Em=615 nm on the EnVision Multilabel Plate Reader (Perkin Elmer) phospho-Tau (Thr212) AlphaLISA signal was used to plot, draw the curve fitting, and determine each compound's EC₅₀ in Prism (GraphPad).

Table 3 shows the activity of representative compounds as provided herein.

TABLE 3

| | pTau |
|---|---|
| | (Thr212) |
| Compound | EC₅₀ (μM) |
| 25 | 0.016 |
| 37 | 0.006 |
| 92 | 0.031 |
| 107 | 0.396 |
| 215 | 0.258 |
| 817 | 0.210 |
| 1194 | 0.002 |
| 1488 | 0.039 |
| 1498 | 0.441 |
| 1502 | 0.936 |
| 1508 | 0.321 |
| 1513 | 0.374 |
| 1514 | 0.082 |
| 1522 | 0.025 |
| 1526 | 0.073 |
| 1529 | 0.413 |
| 1530 | 0.393 |
| 1598 | 0.132 |
| 1640 | 0.199 |
| 1708 | 0.540 |
| 1750 | 0.210 |
| 1782 | 0.305 |
| 2018 | 0.008 |
| 2046 | 0.622 |
| 2054 | 6.245 |

What is claimed is:

1. A compound, or a pharmaceutically acceptable salt thereof, of Formula I: 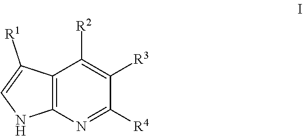
wherein:
R¹ is pyridinyl substituted with 1-4 R⁵;
R² is selected from the group consisting of H, —OR⁷, 5-membered heteroaryl optionally substituted with 1-3 R⁸, and —NHR⁹.
R³ is selected from the group consisting of H, CN, —CO₂H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₚheterocyclyl optionally substituted with 1-10 R¹¹, -(5-9 membered heteroaryl) optionally substituted with 1-10 R¹²; phenyl optionally substituted with 1-5 R¹³, —NHheteroaryl optionally substituted with 1-10 R¹⁴; —C(═O)NHR¹⁵, —NH(C═O)R¹⁶, and —C(═O)R¹⁷;
R⁴ is selected from the group consisting of H, halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, and —C(═O)R²⁵;
with the proviso that at least two of R², R³, and R⁴ are H;
each R⁵ is independently selected from the group consisting of —OR²⁶, -heterocyclyl optionally substituted with 1-10 R²⁷, and —NHheterocyclyl optionally substituted with 1-10 R²⁸;
R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R⁹ is —CH₂(6-membered heteroaryl) optionally substituted with 1-4 R³⁹;
R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R¹³ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R¹⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
R¹⁵ is selected from the group consisting of -heterocyclyl optionally substituted with 1-10 R³⁷ and -heteroaryl optionally substituted with 1-10 R³⁹;
R¹⁶ is selected from the group consisting of -heterocyclyl optionally substituted with 1-10 R³⁷ and -heteroaryl optionally substituted with 1-10 R³⁹;
R¹⁷ is selected from the group consisting of -heterocyclyl optionally substituted with 1-10 R³⁷ and -heteroaryl optionally substituted with 1-10 R³⁹;
R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;
each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R²³ is selected from the group consisting of -heterocyclyl optionally substituted with 1-10 R³⁷ and -heteroaryl optionally substituted with 1-10 R³⁹;
R²⁴ is selected from the group consisting of -heterocyclyl optionally substituted with 1-10 R³⁷ and -heteroaryl optionally substituted with 1-10 R³⁹;
R²⁵ is selected from the group consisting of -heterocyclyl optionally substituted with 1-10 R³⁷ and -heteroaryl optionally substituted with 1-10 R³⁹;
each R²⁶ is unsubstituted —(C₁₋₉ haloalkyl);
each R²⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;
each p is independently 0 or 1; and
with the proviso that Formula I is not a structure selected from the group consisting of:

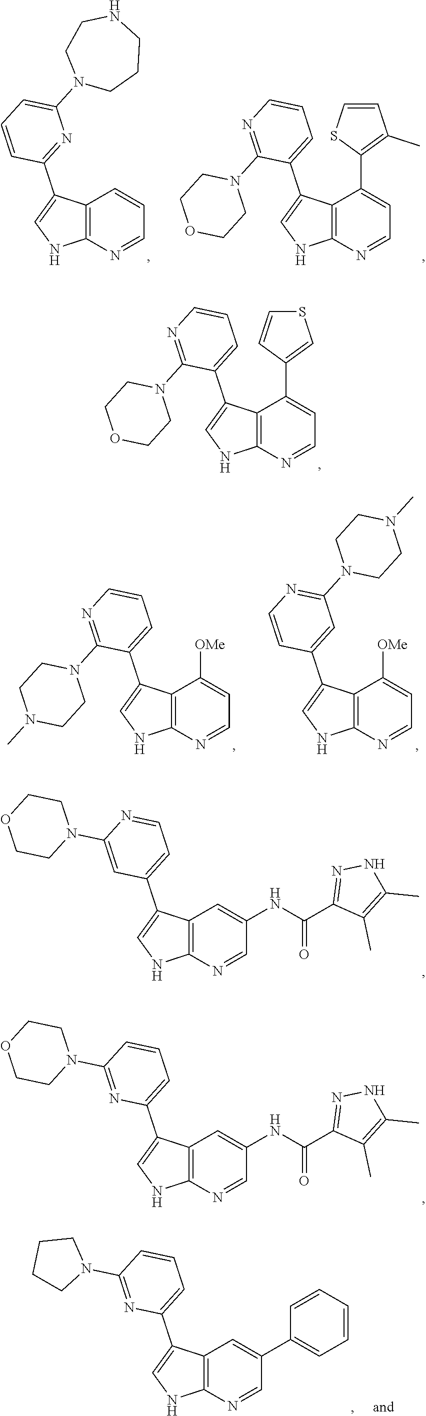

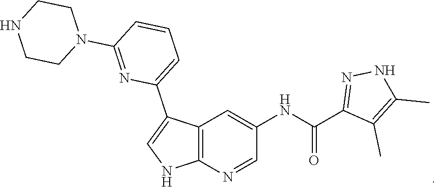 [SMILES: Cc1[nH]nc(C(=O)Nc2cnc3[nH]cc(-c4cccc(N5CCNCC5)n4)c3c2)c1C]

2. The compound of claim 1, wherein R¹ is 4-pyridine substituted with one R⁵.

3. The compound of claim 1, wherein each R⁵ is independently selected from the group consisting of —OMe and -heterocyclyl optionally substituted with one R²⁷.

4. The compound of claim 1, wherein R³ and R⁴ are H and R² is selected from the group consisting of —OMe and a 5-membered heteroaryl optionally substituted with one R⁸.

5. The compound of claim 1, wherein R² and R⁴ are H and R³ is selected from the group consisting of -heterocyclyl optionally substituted with one R¹¹, -(5-6 membered heteroaryl) optionally substituted with 1-2 R¹²; —C(═O)NHR¹⁵, —NH(C═O)R¹⁶, and —C(═O)R¹⁷.

6. The compound of claim 1, wherein R² and R³ are H and R⁴ is selected from the group consisting of -carbocyclyl, —C(═O)NHR²³, and —NH(C═O)R²⁴.

7. A compound, or a pharmaceutically acceptable salt thereof, of Formula Ia: 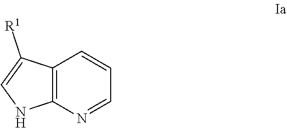
wherein:
R¹ is selected from the heteroaryl group consisting of: 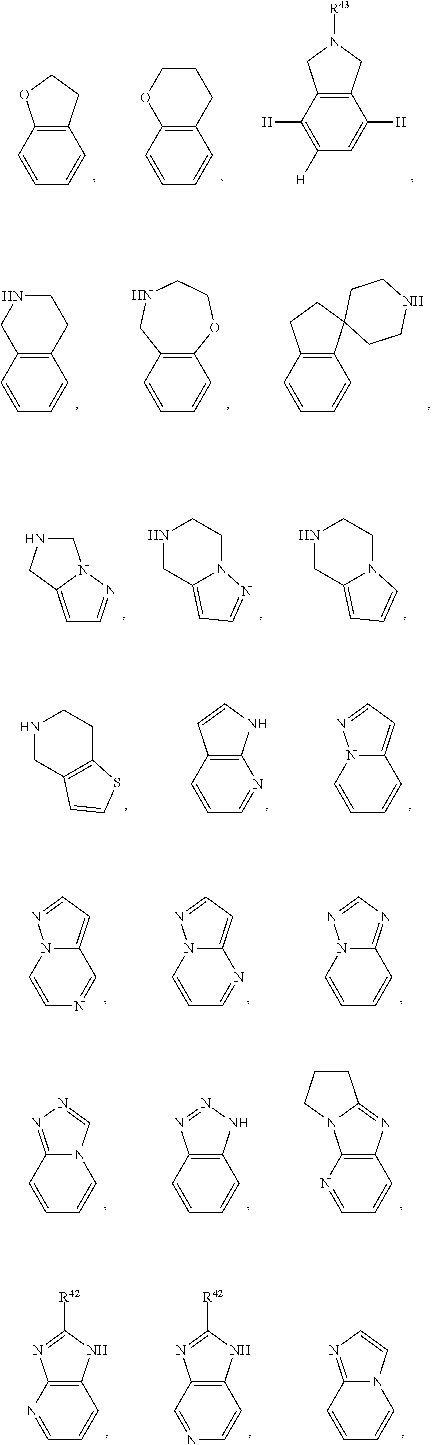 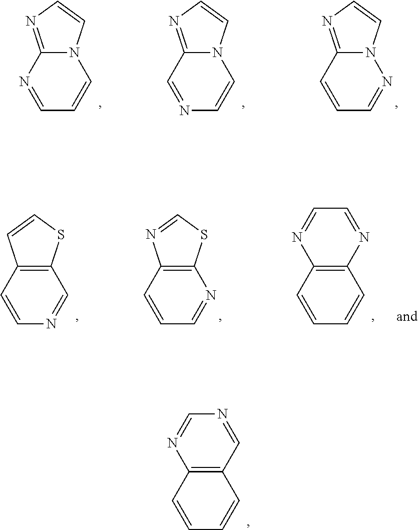
and optionally substituted with 1-10 R⁶; and 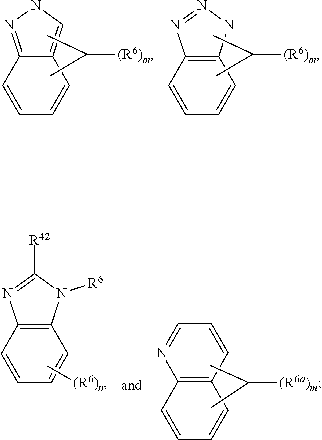
wherein a carbon atom on an aromatic ring of the heteroaryl form the bond with 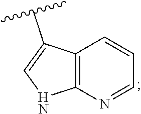 [SMILES: *c1c[nH]c2ncccc12]
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;
alternatively, two R⁶ attached to the same carbon atom are taken together to form a carbonyl group;
alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;
each R^{6a} is independently selected from the group consisting of —OR^{29a}, —CH₂OH, -heterocyclyl optionally substituted with 1-10 R³⁰, —CH₂N(R³⁴)₂, —C(═O)N(R³⁵)₂, and —C(═O)R³⁶;
each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R^{29a} is -heterocyclyl optionally substituted with 1-10 R³⁷;
each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;
each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;
each R³⁶ is -heterocyclyl optionally substituted with 1-10 R⁴¹;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;
each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;
alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;
each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each n is independently 0 to 3;
each m is independently 1 to 6;
each p is independently 0 or 1; and
with the proviso that Formula Ia is not a structure selected from the group consisting of:

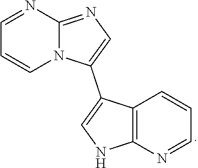 [SMILES: c1cnc2[nH]cc(-c3cnc4ncccn34)c2c1]

8. The compound of claim 7, wherein R¹ is selected from the group consisting of: 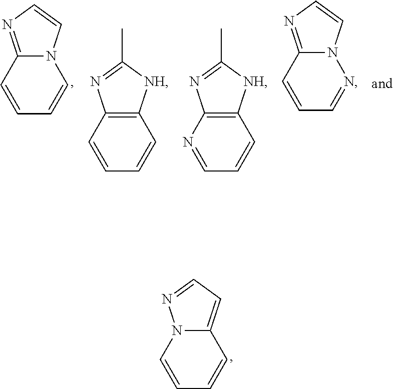
optionally substituted with 1-2 R⁶.

9. The compound of claim 8, wherein each R⁶ is independently selected from the group consisting of unsubstituted —(C₁₋₃ alkyl), -heterocyclyl optionally substituted with one R³⁰, -(5-6 membered heteroaryl) optionally substituted with 1-2 R³², and —C(═O)NHR³⁵.

10. A compound, or a pharmaceutically acceptable salt thereof, of Formula Ib: 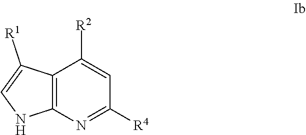
R¹ is 7-14-membered heteroaryl optionally substituted with 1-10 R⁶;
with the proviso that when the heteroaryl is selected from the group consisting of 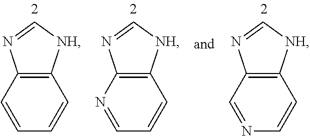
that the 2-position is substituted with R⁴²;
wherein a carbon atom on an aromatic ring of the heteroaryl form the bond with 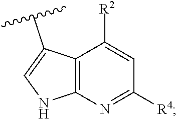
R² is selected from the group consisting of H, —OR⁷, 5-membered heteroaryl optionally substituted with 1-3 R⁸, and —NHR⁹;
R⁴ is selected from the group consisting of H, halide, CN, —OR¹⁸, —CH₂OH, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -(5-9 membered heteroaryl) optionally substituted with 1-10 R¹⁹; phenyl optionally substituted with 1-5 R²⁰, —NHphenyl optionally substituted with 1-5 R²¹, —(CH₂)ₚcarbocyclyl optionally substituted with 1-12 R²², —C(═O)NHR²³, —NH(C═O)R²⁴, and —C(═O)R²⁵;
with the proviso that one of R² and R⁴ is H and the other of R² and R⁴ is not H;
each R⁶ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;
alternatively, two R⁶ attached to the same carbon atom are taken together to form a carbonyl group;
alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;
R⁷ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁸ is independently selected from the group consisting of H, halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R⁹ is —CH₂(6-membered heteroaryl) optionally substituted with 1-4 R³⁹;
R¹⁸ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —(CH₂)ₚcarbocyclyl optionally substituted with 1-12 R³⁸;
each R¹⁹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R²¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R²² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R²³ is selected from the group consisting of -heterocyclyl optionally substituted with 1-10 R³⁷ and -heteroaryl optionally substituted with 1-10 R³⁹;
R²⁴ is selected from the group consisting of -heterocyclyl optionally substituted with 1-10 R³⁷ and -heteroaryl optionally substituted with 1-10 R³⁹;
R²⁵ is selected from the group consisting of -heterocyclyl optionally substituted with 1-10 R³⁷ and -heteroaryl optionally substituted with 1-10 R³⁹;
each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;
each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;
each R³⁶ is -heterocyclyl optionally substituted with 1-10 R⁴¹;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;
each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;
alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;
each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each m is independently 1 to 6;
each p is independently 0 or 1; and
with the proviso that Formula Ib is not a structure selected from the group consisting of:

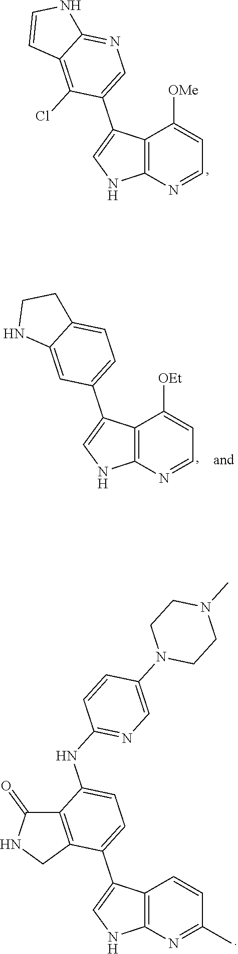 [SMILES: COc1ccnc2[nH]cc(-c3cnc4[nH]ccc4c3Cl)c12 ; CCOc1ccnc2[nH]cc(-c3ccc4c(c3)NCC4)c12 ; Cc1ccc2c(-c3ccc(Nc4ccc(N5CCN(C)CC5)cn4)c4c3CNC4=O)c[nH]c2n1]

11. The compound of claim 10, wherein R¹ is selected from the group consisting of: 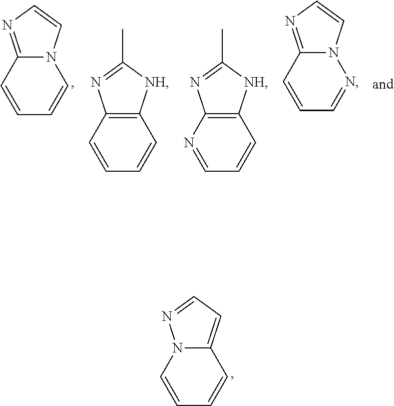
optionally substituted with 1-2 R⁶.

12. The compound claim 11, wherein each R⁶ is independently selected from the group consisting of unsubstituted —(C₁₋₃ alkyl), -heterocyclyl optionally substituted with one R³⁰, -(5-6 membered heteroaryl) optionally substituted with 1-2 R³², and —C(═O)NHR³⁵.

13. The compound of claim 12, wherein R⁴ is H and R² is selected from the group consisting of —OMe and a 5-membered heteroaryl optionally substituted with one R⁸.

14. The compound of claim 12, wherein R² is H and R⁴ is selected from the group consisting of -carbocyclyl, —C(═O)NHR²³, and —NH(C═O)R²⁴.

15. A compound, or a pharmaceutically acceptable salt thereof, of Formula Ie: 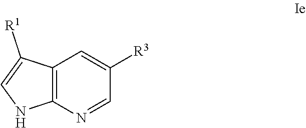
wherein:
R¹ is selected from the heteroaryl group consisting of: 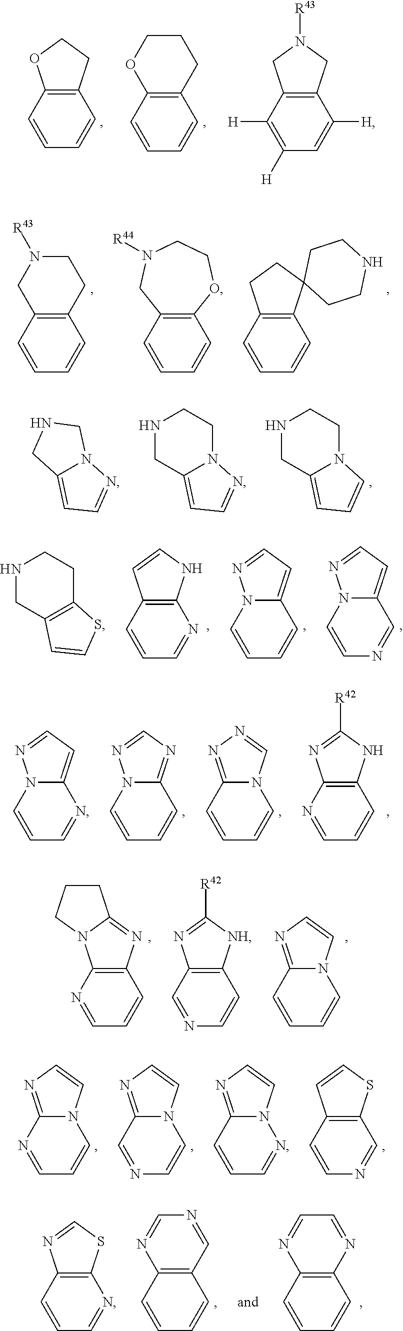
and optionally substituted with 1-10 R⁶; and 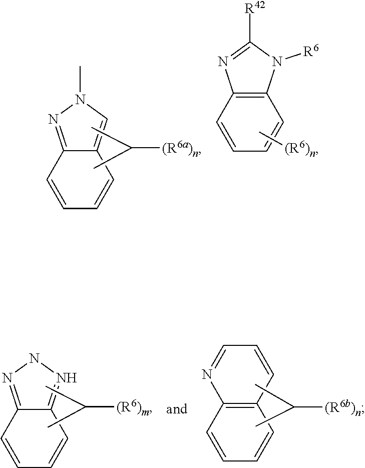
with the proviso that when R¹ is 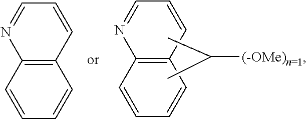
R³ is not halide or methyl;
wherein a carbon atom on an aromatic ring of the heteroaryl form the bond with 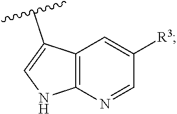
R³ is selected from the group consisting of halide, CN, —CO₂H, —OR¹⁰, unsubstituted —(C₁₋₉ alkyl), unsubstituted —(C₂₋₉ alkenyl), unsubstituted —(C₂₋₉alkynyl), unsubstituted —(C₁₋₉haloalkyl), —(CH₂)ₚheterocyclyl optionally substituted with 1-10 R¹¹, -(5-9 membered heteroaryl) optionally substituted with 1-10 R¹²; phenyl optionally substituted with 1-5 R¹³, —NHheteroaryl optionally substituted with 1-10 R¹⁴; —C(═O)NHR¹⁵, —NH(C═O)R¹⁶, and —C(═O)R¹⁷;
each R⁶ is independently selected from the group consisting of halide, unsubstituted —(C₁₋₉ alkyl), unsubstituted —(C₂₋₉ alkenyl), unsubstituted —(C₂₋₉alkynyl), unsubstituted —(C₁₋₉haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;
alternatively, two R⁶ attached to the same carbon atom are taken together to form a carbonyl group;
alternatively, two R⁶ attached to the same carbon atom are taken together to form a ring which is selected from the group consisting of -heterocyclyl optionally substituted with 1-10 R³⁷ and -carbocyclyl optionally substituted with 1-12 R³⁸;
each R^{6a} is independently selected from the group consisting of halide, unsubstituted —(C₂₋₉ alkyl), unsubstituted —(C₂₋₉ alkenyl), unsubstituted —(C₂₋₉ alkynyl), unsubstituted —(C₁₋₉ haloalkyl), —OR²⁹, —CH₂OH, -heterocyclyl optionally substituted with 1-10 R³⁰, —NHheterocyclyl optionally substituted with 1-10 R³¹, -heteroaryl optionally substituted with 1-10 R³²; —NHheteroaryl optionally substituted with 1-10 R³³; —NHCH₂heteroaryl optionally substituted with 1-10 R³³; —CH₂N(R³⁴)₂, —C(═O)N(R³⁵)₂, and —C(═O)R³⁶;
each R^{6b} is independently selected from the group consisting of —OR²⁹, —CH₂OH, -heterocyclyl optionally substituted with 1-10 R³⁰, —CH₂N(R³⁴)₂, —C(═O)N(R³⁵)₂, and —C(═O)R³⁶;
R¹⁰ is selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R¹³ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R¹⁴ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
R¹⁵ is selected from the group consisting of -heterocyclyl optionally substituted with 1-10 R³⁷ and -heteroaryl optionally substituted with 1-10 R³⁹;
R¹⁶ is selected from the group consisting of -heterocyclyl optionally substituted with 1-10 R³⁷ and -heteroaryl optionally substituted with 1-10 R³⁹;
R¹⁷ is selected from the group consisting of -heterocyclyl optionally substituted with 1-10 R³⁷ and -heteroaryl optionally substituted with 1-10 R³⁹;
each R²⁹ is independently selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R³⁰ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
alternatively, two R³⁰ attached to the same carbon atom are taken together to form a ring which is selected from the group consisting of -heterocyclyl optionally substituted with 1-10 R³⁷ and -carbocyclyl optionally substituted with 1-12 R³⁸;
each R³¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³² is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³³ is independently selected from the group consisting of halide, —OR⁴⁵, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each R³⁴ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁵ are independently selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl); —(CH₂)ₚheterocyclyl optionally substituted with 1-10 R⁴⁰, and -heteroaryl optionally substituted with 1-10 R³⁹;
each R³⁶ is -heterocyclyl optionally substituted with 1-10 R⁴¹;
each R³⁷ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁸ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R³⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R³⁷, and -heteroaryl optionally substituted with 1-10 R³⁸;
each R⁴⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(CH₂)ₘOMe, —(CH₂)ₚcarbocyclyl optionally substituted with 1-12 R³⁸, and —(CH₂)ₚheteroaryl optionally substituted with 1-10 R³⁹;
each R⁴¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -carbocyclyl optionally substituted with 1-12 R³⁸, and -heterocyclyl optionally substituted with 1-10 R³⁷;
alternatively, two R⁴¹ attached to the same carbon atom are taken together to form a ring which is selected from the group consisting of -heterocyclyl optionally substituted with 1-10 R³⁷ and -carbocyclyl optionally substituted with 1-12 R³⁸;
each R⁴² is independently selected from the group consisting of unsubstituted —(C₁₋₉ alkyl), unsubstituted —(C₂₋₉ alkenyl), unsubstituted —(C₂₋₉ alkynyl), and unsubstituted —(C₁₋₉haloalkyl);
each R⁴³ is H;
each R⁴⁴ is independently selected from the group consisting of H, unsubstituted —(C₂₋₉ alkyl), unsubstituted —(C₂₋₉ alkenyl), unsubstituted —(C₂₋₉ alkynyl), and unsubstituted —(C₁₋₉ haloalkyl);
each R⁴⁵ is independently selected from the group consisting of unsubstituted —(C₂₋₉ alkyl), unsubstituted —(C₂₋₉ alkenyl), unsubstituted —(C₂₋₉ alkynyl), unsubstituted —(C₁₋₉ haloalkyl), and -heterocyclyl optionally substituted with 1-10 R³⁷;
each n is independently 0 to 6;
each m is independently 1 to 6;
each p is independently 0 or 1; and
with the proviso that Formula Ie is not a structure selected from the group consisting of:

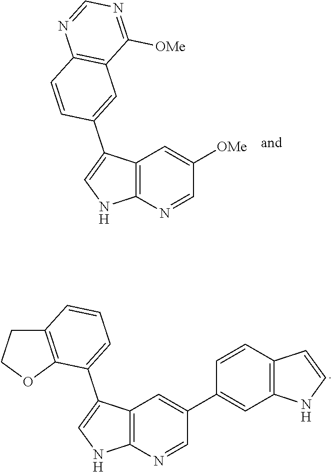 [SMILES: COc1cnc2[nH]cc(-c3ccc4ncnc(OC)c4c3)c2c1 ; c1cc2c(c(-c3c[nH]c4ncc(-c5ccc6cc[nH]c6c5)cc34)c1)OCC2]

16. The compound of claim 15, wherein R¹ is selected from the group consisting of: 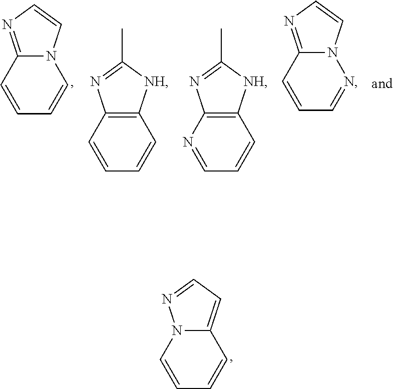
optionally substituted with 1-2 R⁶.

17. The compound of claim 16, wherein each R⁶ is independently selected from the group consisting of unsubstituted —(C₁₋₃ alkyl), -heterocyclyl optionally substituted with one R³⁰, -(5-6 membered heteroaryl) optionally substituted with 1-2 R³², and —C(═O)NHR³⁵.

18. The compound of claim 17, wherein R³ is selected from the group consisting of -heterocyclyl optionally substituted with one R¹¹, -(5-6 membered heteroaryl) optionally substituted with 1-2 R¹²; —C(═O)NHR¹⁵, —NH(C═O)R¹⁶, and —C(═O)R¹⁷.

19. The compound according to claim 1, wherein the compound of Formula I is selected from the group consisting of:
3-(2-(4-methylpiperazin-1-yl)pyridin-4-yl)-1H-pyrrolo[2,3-b]pyridine [68];
4-(1-methyl-1H-pyrazol-4-yl)-3-(2-(4-methylpiperazin-1-yl)pyridin-4-yl)-1H-pyrrolo[2,3-b]pyridine [175];
5-methyl-3-(2-(4-methylpiperazin-1-yl)pyridin-4-yl)-1H-pyrrolo[2,3-b]pyridine [301];
5-(1-methyl-1,2,3,6-tetrahydropyridin-4-yl)-3-(2-(4-methylpiperazin-1-yl)pyridin-4-yl)-1H-pyrrolo[2,3-b]pyridine [343];
5-(4-methylpiperazin-1-yl)-3-(2-(4-methylpiperazin-1-yl)pyridin-4-yl)-1H-pyrrolo[2,3-b]pyridine [385];
5-((4-methylpiperazin-1-yl)methyl)-3-(2-(4-methylpiperazin-1-yl)pyridin-4-yl)-1H-pyrrolo[2,3-b]pyridine [427];
3-(2-(4-methylpiperazin-1-yl)pyridin-4-yl)-N-(1-methylpiperidin-4-yl)-1H-pyrrolo[2,3-b]pyridine-5-carboxamide [469];
(4-methylpiperazin-1-yl)(3-(2-(4-methylpiperazin-1-yl)pyridin-4-yl)-1H-pyrrolo[2,3-b]pyridin-5-yl)methanone [511];
(3-methyl-3,8-diazabicyclo[3.2.1]octan-8-yl)(3-(2-(4-methylpiperazin-1-yl)pyridin-4-yl)-1H-pyrrolo[2,3-b]pyridin-5-yl)methanone [553];
(2-methyl-5,6-dihydroimidazo[1,2-a]pyrazin-7(8H)-yl)(3-(2-(4-methylpiperazin-1-yl)pyridin-4-yl)-1H-pyrrolo[2,3-b]pyridin-5-yl)methanone [595];
1-methyl-N-(3-(2-(4-methylpiperazin-1-yl)pyridin-4-yl)-1H-pyrrolo[2,3-b]pyridin-5-yl)piperidine-4-carboxamide [637];
3-(2-(4-methylpiperazin-1-yl)pyridin-4-yl)-N-(1-(piperidin-4-yl)-1H-pyrazol-4-yl)-1H-pyrrolo[2,3-b]pyridine-5-carboxamide [679];
3-(2-(4-methylpiperazin-1-yl)pyridin-4-yl)-N-(1-(1-methylpiperidin-4-yl)-1H-pyrazol-4-yl)-1H-pyrrolo[2,3-b]pyridine-5-carboxamide [721];
N-(5-fluoropyridin-3-yl)-3-(2-(4-methylpiperazin-1-yl)pyridin-4-yl)-1H-pyrrolo[2,3-b]pyridine-5-carboxamide [763];
N-(6-(4-methylpiperazin-1-yl)pyridin-3-yl)-3-(2-(4-methylpiperazin-1-yl)pyridin-4-yl)-1H-pyrrolo[2,3-b]pyridine-5-carboxamide [805];
N-(3-(2-(4-methylpiperazin-1-yl)pyridin-4-yl)-1H-pyrrolo[2,3-b]pyridin-5-yl)-2-(piperazin-1-yl)isonicotinamide [847];
N,3-bis(2-(4-methylpiperazin-1-yl)pyridin-4-yl)-1H-pyrrolo[2,3-b]pyridin-5-amine [889];
3-(2-(4-methylpiperazin-1-yl)pyridin-4-yl)-5-phenyl-1H-pyrrolo[2,3-b]pyridine [931];
3-(2-(4-methylpiperazin-1-yl)pyridin-4-yl)-5-(o-tolyl)-1H-pyrrolo[2,3-b]pyridine [973];
5-(3,4-difluoro-5-(piperazin-1-yl)phenyl)-3-(2-(4-methylpiperazin-1-yl)pyridin-4-yl)-1H-pyrrolo[2,3-b]pyridine [1015];
5-(5-(1-methyl-1H-pyrazol-4-yl)-1H-pyrrolo[2,3-b]pyridin-3-yl)isoindolin-1-one [1060];
3-(2-(4-methylpiperazin-1-yl)pyridin-4-yl)-5-(1,3,5-trimethyl-1H-pyrazol-4-yl)-1H-pyrrolo[2,3-b]pyridine [1099]; and
5-methyl-3-(3-(2-(4-methylpiperazin-1-yl)pyridin-4-yl)-1H-pyrrolo[2,3-b]pyridin-5-yl)-4,5,6,7-tetrahydropyrazolo[1,5-a]pyrazine [1141];
3-(2-(4-methylpiperazin-1-yl)pyridin-4-yl)-5-(pyridin-3-yl)-1H-pyrrolo[2,3-b]pyridine [1183];
3-(2-(4-methylpiperazin-1-yl)pyridin-4-yl)-5-(2-methylpyridin-3-yl)-1H-pyrrolo[2,3-b]pyridine [1225];
3-(2-(4-methylpiperazin-1-yl)pyridin-4-yl)-5-(4-methylpyridin-3-yl)-1H-pyrrolo[2,3-b]pyridine [1267];
5-(2,4-dimethylpyridin-3-yl)-3-(2-(4-methylpiperazin-1-yl)pyridin-4-yl)-1H-pyrrolo[2,3-b]pyridine [1309];
5-(6-(4-methylpiperazin-1-yl)pyridin-3-yl)-3-(2-(4-methylpiperazin-1-yl)pyridin-4-yl)-1H-pyrrolo[2,3-b]pyridine [1351];
3,5-bis(2-(4-methylpiperazin-1-yl)pyridin-4-yl)-1H-pyrrolo[2,3-b]pyridine [1393];
6-chloro-3-(2-(4-methylpiperazin-1-yl)pyridin-4-yl)-1H-pyrrolo[2,3-b]pyridine [1435];
6-methyl-3-(2-(4-methylpiperazin-1-yl)pyridin-4-yl)-1H-pyrrolo[2,3-b]pyridine [1477];
5-(6-cyclopropyl-1H-pyrrolo[2,3-b]pyridin-3-yl)isoindolin-1-one [1522];
6-cyclobutyl-3-(2-(4-methylpiperazin-1-yl)pyridin-4-yl)-1H-pyrrolo[2,3-b]pyridine [1561];
6-cyclopentyl-3-(2-(4-methylpiperazin-1-yl)pyridin-4-yl)-1H-pyrrolo[2,3-b]pyridine [1603];
6-(cyclobutylmethyl)-3-(2-(4-methylpiperazin-1-yl)pyridin-4-yl)-1H-pyrrolo[2,3-b]pyridine [1645];
6-methoxy-3-(2-(4-methylpiperazin-1-yl)pyridin-4-yl)-1H-pyrrolo[2,3-b]pyridine [1687];
6-(difluoromethoxy)-3-(2-(4-methylpiperazin-1-yl)pyridin-4-yl)-1H-pyrrolo[2,3-b]pyridine [1729];
6-ethoxy-3-(2-(4-methylpiperazin-1-yl)pyridin-4-yl)-1H-pyrrolo[2,3-b]pyridine [1771];
(3-(2-(4-methylpiperazin-1-yl)pyridin-4-yl)-1H-pyrrolo[2,3-b]pyridin-6-yl)methanol [1813];
1-methyl-N-(3-(2-(4-methylpiperazin-1-yl)pyridin-4-yl)-1H-pyrrolo[2,3-b]pyridin-6-yl)piperidine-4-carboxamide [1855];
N-(3-(2-(4-methylpiperazin-1-yl)pyridin-4-yl)-1H-pyrrolo[2,3-b]pyridin-6-yl)isonicotinamide [1897];
N-(3-(4-methylpiperazin-1-yl)phenyl)-3-(2-(4-methylpiperazin-1-yl)pyridin-4-yl)-1H-pyrrolo[2,3-b]pyridin-6-amine [1939];
6-(1-methyl-1H-pyrazol-4-yl)-3-(2-(4-methylpiperazin-1-yl)pyridin-4-yl)-1H-pyrrolo[2,3-b]pyridine [1981]; and
3-(2-(4-methylpiperazin-1-yl)pyridin-4-yl)-6-phenyl-1H-pyrrolo[2,3-b]pyridine [2023]; or a pharmaceutically acceptable salt thereof.

20. The compound according to claim 7, wherein the compound of Formula Ia is selected from the group consisting of:
1H,1′H-3,4′-bipyrrolo[2,3-b]pyridine [2];
2-(1H-pyrrolo[2,3-b]pyridin-3-yl)thiazolo[5,4-b]pyridine [4];
2-(1H-pyrrolo[2,3-b]pyridin-3-yl)thieno[2,3-c]pyridine [5];
1H,1′H-2,3′-bipyrrolo[2,3-b]pyridine [6];
2-(1H-pyrrolo[2,3-b]pyridin-3-yl)-6,7-dihydropyrazolo[1,5-a]pyrazin-4(5H)-one [8];
6,6-dimethyl-2-(1H-pyrrolo[2,3-b]pyridin-3-yl)-5,6-dihydro-4H-imidazo[1,5-b]pyrazol-4-one [9];
7-(1H-pyrrolo[2,3-b]pyridin-3-yl)-3,4-dihydropyrrolo[1,2-a]pyrazin-1(2H)-one [10];
2-(1H-pyrrolo[2,3-b]pyridin-3-yl)-6,7-dihydrothieno[3,2-c]pyridin-4(5H)-one [11];
6-(1H-pyrrolo[2,3-b]pyridin-3-yl)imidazo[1,2-a]pyridine [12];
8-fluoro-2-methyl-6-(1H-pyrrolo[2,3-b]pyridin-3-yl)imidazo[1,2-a]pyridine [13];
N-(1-methylpiperidin-4-yl)-6-(1H-pyrrolo[2,3-b]pyridin-3-yl)imidazo[1,2-a]pyridine-3-carboxamide [14];
N-methyl-N-(1-methylpiperidin-4-yl)-6-(1H-pyrrolo[2,3-b]pyridin-3-yl)imidazo[1,2-a]pyridine-3-carboxamide [15];
N-(2,2-difluoroethyl)-6-(1H-pyrrolo[2,3-b]pyridin-3-yl)imidazo[1,2-a]pyridine-3-carboxamide [16];
N-(2,2-difluoroethyl)-N-methyl-6-(1H-pyrrolo[2,3-b]pyridin-3-yl)imidazo[1,2-a]pyridine-3-carboxamide [17];
1-(6-(1H-pyrrolo[2,3-b]pyridin-3-yl)imidazo[1,2-a]pyridin-3-yl)-N,N-dimethylmethanamine [18];
6-(1H-pyrrolo[2,3-b]pyridin-3-yl)imidazo[1,2-a]pyrimidine [19];
6-(1H-pyrrolo[2,3-b]pyridin-3-yl)imidazo[1,2-a]pyrazine [20];
(6-(1H-pyrrolo[2,3-b]pyridin-3-yl)imidazo[1,2-a]pyrazin-3-yl)(4-methylpiperazin-1-yl)methanone [21];
4-fluoro-1-isopropyl-2-methyl-6-(1H-pyrrolo[2,3-b]pyridin-3-yl)-1H-benzo[d]imidazole [22];
4-fluoro-2-methyl-1-(piperidin-4-yl)-6-(1H-pyrrolo[2,3-b]pyridin-3-yl)-1H-benzo[d]imidazole [23];
4-fluoro-2-methyl-1-(1-methylpiperidin-4-yl)-6-(1H-pyrrolo[2,3-b]pyridin-3-yl)-1H-benzo[d]imidazole [24];
1-isopropyl-2-methyl-6-(1H-pyrrolo[2,3-b]pyridin-3-yl)-1H-imidazo[4,5-b]pyridine [25];
3-isopropyl-2-methyl-5-(1H-pyrrolo[2,3-b]pyridin-3-yl)-3H-imidazo[4,5-b]pyridine [26];
3-isopropyl-2-methyl-5-(1H-pyrrolo[2,3-b]pyridin-3-yl)-3H-imidazo[4,5-b]pyridine [27];
2-(1H-pyrrolo[2,3-b]pyridin-3-yl)-7,8-dihydro-6H-pyrrolo[2′,1′:2,3]imidazo[4,5-b]pyridine [28];
6-(1H-pyrrolo[2,3-b]pyridin-3-yl)-[1,2,4]triazolo[1,5-a]pyridine [29];
7-(1H-pyrrolo[2,3-b]pyridin-3-yl)-[1,2,4]triazolo[1,5-a]pyridine [30];
3-methyl-6-(1H-pyrrolo[2,3-b]pyridin-3-yl)-[1,2,4]triazolo[4,3-a]pyridine [32];
6-(1H-pyrrolo[2,3-b]pyridin-3-yl)imidazo[1,2-b]pyridazine [33];
3-methyl-6-(1H-pyrrolo[2,3-b]pyridin-3-yl)imidazo[1,2-b]pyridazine [34];
(6-(1H-pyrrolo[2,3-b]pyridin-3-yl)imidazo[1,2-b]pyridazin-3-yl)methanol [35];
3-(pyrazolo[1,5-a]pyridin-5-yl)-1H-pyrrolo[2,3-b]pyridine [36];
3-(3-(1-methyl-1H-pyrazol-4-yl)pyrazolo[1,5-a]pyridin-5-yl)-1H-pyrrolo[2,3-b]pyridine [37];
3-(3-(pyridin-3-yl)pyrazolo[1,5-a]pyridin-5-yl)-1H-pyrrolo[2,3-b]pyridine [38];
N-(2,2-difluoroethyl)-5-(1H-pyrrolo[2,3-b]pyridin-3-yl)pyrazolo[1,5-a]pyridine-3-carboxamide [39];
(R)-5-(1H-pyrrolo[2,3-b]pyridin-3-yl)-N-(1,1,1-trifluoropropan-2-yl)pyrazolo[1,5-a]pyridine-3-carboxamide [40];
N-((3-fluoroazetidin-3-yl)methyl)-5-(1H-pyrrolo[2,3-b]pyridin-3-yl)pyrazolo[1,5-a]pyridine-3-carboxamide [41];
N-((3-fluoro-1-methylazetidin-3-yl)methyl)-5-(1H-pyrrolo[2,3-b]pyridin-3-yl)pyrazolo[1,5-a]pyridine-3-carboxamide [42];
N-((3R,4R)-3-fluoropiperidin-4-yl)-5-(1H-pyrrolo[2,3-b]pyridin-3-yl)pyrazolo[1,5-a]pyridine-3-carboxamide [43];
N-((3R,4R)-3-fluoro-1-methylpiperidin-4-yl)-5-(1H-pyrrolo[2,3-b]pyridin-3-yl)pyrazolo[1,5-a]pyridine-3-carboxamide [44];
N-(1-methylpiperidin-4-yl)-5-(1H-pyrrolo[2,3-b]pyridin-3-yl)pyrazolo[1,5-a]pyridine-3-carboxamide [45];
N-methyl-N-(1-methylpiperidin-4-yl)-5-(1H-pyrrolo[2,3-b]pyridin-3-yl)pyrazolo[1,5-a]pyridine-3-carboxamide [46];
N-(1-cyclopropylpiperidin-4-yl)-5-(1H-pyrrolo[2,3-b]pyridin-3-yl)pyrazolo[1,5-a]pyridine-3-carboxamide [47];
N-(1-(cyclopropylmethyl)piperidin-4-yl)-5-(1H-pyrrolo[2,3-b]pyridin-3-yl)pyrazolo[1,5-a]pyridine-3-carboxamide [48];
N-(1-(2,2-difluoropropyl)piperidin-4-yl)-5-(1H-pyrrolo[2,3-b]pyridin-3-yl)pyrazolo[1,5-a]pyridine-3-carboxamide [49];
N-(1-(2-methoxyethyl)piperidin-4-yl)-5-(1H-pyrrolo[2,3-b]pyridin-3-yl)pyrazolo[1,5-a]pyridine-3-carboxamide [50];
N-(1-(pyridin-2-ylmethyl)piperidin-4-yl)-5-(1H-pyrrolo[2,3-b]pyridin-3-yl)pyrazolo[1,5-a]pyridine-3-carboxamide [51];
N-(pyridin-3-yl)-5-(1H-pyrrolo[2,3-b]pyridin-3-yl)pyrazolo[1,5-a]pyridine-3-carboxamide [52];
(5-(1H-pyrrolo[2,3-b]pyridin-3-yl)pyrazolo[1,5-a]pyridin-3-yl)(piperidin-1-yl)methanone [53];
(5-(1H-pyrrolo[2,3-b]pyridin-3-yl)pyrazolo[1,5-a]pyridin-3-yl)(4-(4-methylpiperazin-1-yl)piperidin-1-yl)methanone [54];
(5-(1H-pyrrolo[2,3-b]pyridin-3-yl)pyrazolo[1,5-a]pyridin-3-yl)(4-methylpiperazin-1-yl)methanone [55];
(5-(1H-pyrrolo[2,3-b]pyridin-3-yl)pyrazolo[1,5-a]pyridin-3-yl)(4-cyclopropylpiperazin-1-yl)methanone [56];
(5-(1H-pyrrolo[2,3-b]pyridin-3-yl)pyrazolo[1,5-a]pyridin-3-yl)(4,7-diazaspiro[2.5]octan-7-yl)methanone [57];
(5-(1H-pyrrolo[2,3-b]pyridin-3-yl)pyrazolo[1,5-a]pyridin-3-yl)(8-methyl-3,8-diazabicyclo[3.2.1]octan-3-yl)methanone [58];
(5-(1H-pyrrolo[2,3-b]pyridin-3-yl)pyrazolo[1,5-a]pyridin-3-yl)(3-methyl-3,8-diazabicyclo[3.2.1]octan-8-yl)methanone [59];
(5-(1H-pyrrolo[2,3-b]pyridin-3-yl)pyrazolo[1,5-a]pyridin-3-yl)(morpholino)methanone [60];
(5-(1H-pyrrolo[2,3-b]pyridin-3-yl)pyrazolo[1,5-a]pyridin-3-yl)(2,6-diazaspiro[3.3]heptan-2-yl)methanone [61];
(5-(1H-pyrrolo[2,3-b]pyridin-3-yl)pyrazolo[1,5-a]pyridin-3-yl)(6-methyl-2,6-diazaspiro[3.3]heptan-2-yl)methanone [62];
(5-(1H-pyrrolo[2,3-b]pyridin-3-yl)pyrazolo[1,5-a]pyridin-3-yl)(hexahydropyrrolo[3,4-c]pyrrol-2(1H)-yl)methanone [63];
(5-(1H-pyrrolo[2,3-b]pyridin-3-yl)pyrazolo[1,5-a]pyridin-3-yl)(5-methylhexahydropyrrolo[3,4-c]pyrrol-2(1H)-yl)methanone [64];
5-(1H-pyrrolo[2,3-b]pyridin-3-yl)pyrazolo[1,5-a]pyrimidine [65];
3-(3,3-dimethyl-2,3-dihydrobenzofuran-5-yl)-1H-pyrrolo[2,3-b]pyridine [72];
6-(1H-pyrrolo[2,3-b]pyridin-3-yl)spiro[indene-1,4′-piperidin]-3(2H)-one [73];
1′-methyl-6-(1H-pyrrolo[2,3-b]pyridin-3-yl)spiro[indene-1,4′-piperidin]-3(2H)-one [74];
5-(1H-pyrrolo[2,3-b]pyridin-3-yl)isoindolin-1-one [75];
3,3-dimethyl-5-(1H-pyrrolo[2,3-b]pyridin-3-yl)isoindolin-1-one [77];
6′-(1H-pyrrolo[2,3-b]pyridin-3-yl)spiro[cyclohexane-1,1′-isoindolin]-3′-one [78];
3-(chroman-6-yl)-1H-pyrrolo[2,3-b]pyridine [79];
2,2-dimethyl-7-(1H-pyrrolo[2,3-b]pyridin-3-yl)chroman-4-one [80];
6-(1H-pyrrolo[2,3-b]pyridin-3-yl)-3,4-dihydroisoquinolin-1(2H)-one [81];
4,4-dimethyl-6-(1H-pyrrolo[2,3-b]pyridin-3-yl)-3,4-dihydroisoquinolin-1(2H)-one [82];
7-(1H-pyrrolo[2,3-b]pyridin-3-yl)spiro[chromane-2,4′-piperidin]-4-one [83];
8-(1H-pyrrolo[2,3-b]pyridin-3-yl)-3,4-dihydrobenzo[f][1,4]oxazepin-5(2H)-one [84];
4-methyl-8-(1H-pyrrolo[2,3-b]pyridin-3-yl)-3,4-dihydrobenzo[f][1,4]oxazepin-5(2H)-one [85];
9-fluoro-8-(1H-pyrrolo[2,3-b]pyridin-3-yl)-3,4-dihydrobenzo[f][1,4]oxazepin-5(2H)-one [86];
(6-(1H-pyrrolo[2,3-b]pyridin-3-yl)quinolin-4-yl)(4,7-diazaspiro[2.5]octan-7-yl)methanone [88];
N-(1-methylpiperidin-4-yl)-6-(1H-pyrrolo[2,3-b]pyridin-3-yl)quinoline-4-carboxamide [89];
6-(1H-pyrrolo[2,3-b]pyridin-3-yl)quinoxaline [90];
2-((1-methylpiperidin-4-yl)oxy)-7-(1H-pyrrolo[2,3-b]pyridin-3-yl)quinoxaline [91];
N-(1-methylpiperidin-4-yl)-7-(1H-pyrrolo[2,3-b]pyridin-3-yl)quinoxalin-2-amine [92];
6-(1H-pyrrolo[2,3-b]pyridin-3-yl)quinazoline [93];
4-methoxy-6-(1H-pyrrolo[2,3-b]pyridin-3-yl)quinazoline [94];
4-(4-methylpiperazin-1-yl)-6-(1H-pyrrolo[2,3-b]pyridin-3-yl)quinazoline [95];
6-(1H-pyrrolo[2,3-b]pyridin-3-yl)-4-(4,7-diazaspiro[2.5]octan-7-yl)quinazoline [96];
6-(1H-pyrrolo[2,3-b]pyridin-3-yl)-4-(5,8-diazaspiro[3.5]nonan-8-yl)quinazoline [97];
9-(6-(1H-pyrrolo[2,3-b]pyridin-3-yl)quinazolin-4-yl)-6,9-diazaspiro[4.5]decane [98];
4-((1-methylpiperidin-4-yl)oxy)-6-(1H-pyrrolo[2,3-b]pyridin-3-yl)quinazoline [99];
N-(1-methylpiperidin-4-yl)-6-(1H-pyrrolo[2,3-b]pyridin-3-yl)quinazolin-4-amine [100];
N-(6-methoxypyridin-3-yl)-6-(1H-pyrrolo[2,3-b]pyridin-3-yl)quinazolin-4-amine [101];
N-(6-((1-methylpiperidin-4-yl)oxy)pyridin-3-yl)-6-(1H-pyrrolo[2,3-b]pyridin-3-yl)quinazolin-4-amine [102];
N-(pyridin-3-ylmethyl)-6-(1H-pyrrolo[2,3-b]pyridin-3-yl)quinazolin-4-amine [103];
N-((6-(4-methylpiperazin-1-yl)pyridin-3-yl)methyl)-6-(1H-pyrrolo[2,3-b]pyridin-3-yl)quinazolin-4-amine [104];
N-((2-(4-methylpiperazin-1-yl)pyridin-4-yl)methyl)-6-(1H-pyrrolo[2,3-b]pyridin-3-yl)quinazolin-4-amine [105];
N-(1-methylpiperidin-4-yl)-6-(1H-pyrrolo[2,3-b]pyridin-3-yl)quinazoline-4-carboxamide [106];
(5-(1H-pyrrolo[2,3-b]pyridin-3-yl)pyrazolo[1,5-a]pyridin-3-yl)(4-morpholinopiperidin-1-yl)methanone [107]; and
(6-(1H-pyrrolo[2,3-b]pyridin-3-yl)quinazolin-4-yl)(4,7-diazaspiro[2.5]octan-7-yl)methanone [108]; or a pharmaceutically acceptable salt thereof.

21. The compound according to claim 10, wherein the compound of Formula Ib is selected from the group consisting of:
5-(4-methoxy-1H-pyrrolo[2,3-b]pyridin-3-yl)-2-methyl-2H-indazole [109];
2-(4-methoxy-1H-pyrrolo[2,3-b]pyridin-3-yl)thieno[2,3-c]pyridine [110];
7-(4-methoxy-1H-pyrrolo[2,3-b]pyridin-3-yl)-3,4-dihydropyrrolo[1,2-a]pyrazin-1(2H)-one [111];
6-(4-methoxy-1H-pyrrolo[2,3-b]pyridin-3-yl)imidazo[1,2-a]pyridine [112];
N-(2,2-difluoroethyl)-6-(4-methoxy-1H-pyrrolo[2,3-b]pyridin-3-yl)imidazo[1,2-a]pyridine-3-carboxamide [113];
6-(4-methoxy-1H-pyrrolo[2,3-b]pyridin-3-yl)imidazo[1,2-a]pyrimidine [114];
4-fluoro-1-isopropyl-6-(4-methoxy-1H-pyrrolo[2,3-b]pyridin-3-yl)-2-methyl-1H-benzo[d]imidazole [115];
4-fluoro-6-(4-methoxy-1H-pyrrolo[2,3-b]pyridin-3-yl)-2-methyl-1-(1-methylpiperidin-4-yl)-1H-benzo[d]imidazole [116];
1-isopropyl-6-(4-methoxy-1H-pyrrolo[2,3-b]pyridin-3-yl)-2-methyl-1H-imidazo[4,5-b]pyridine [117];
1-isopropyl-6-(4-methoxy-1H-pyrrolo[2,3-b]pyridin-3-yl)-2-methyl-1H-imidazo[4,5-c]pyridine [118];
6-(4-methoxy-1H-pyrrolo[2,3-b]pyridin-3-yl)-[1,2,4]triazolo[1,5-a]pyridine [119];
7-(4-methoxy-1H-pyrrolo[2,3-b]pyridin-3-yl)-[1,2,4]triazolo[1,5-a]pyridine [120];
6-(4-methoxy-1H-pyrrolo[2,3-b]pyridin-3-yl)-1-methyl-1H-benzo[d][1,2,3]triazole [121];
6-(4-methoxy-1H-pyrrolo[2,3-b]pyridin-3-yl)imidazo[1,2-b]pyridazine [122];
4-methoxy-3-(pyrazolo[1,5-a]pyridin-5-yl)-1H-pyrrolo[2,3-b]pyridine [123];
4-methoxy-3-(3-(1-methyl-1H-pyrazol-4-yl)pyrazolo[1,5-a]pyridin-5-yl)-1H-pyrrolo[2,3-b]pyridine [124];
4-methoxy-3-(3-(pyridin-3-yl)pyrazolo[1,5-a]pyridin-5-yl)-1H-pyrrolo[2,3-b]pyridine [125];
N-(2,2-difluoroethyl)-5-(4-methoxy-1H-pyrrolo[2,3-b]pyridin-3-yl)pyrazolo[1,5-a]pyridine-3-carboxamide [126];
(R)-5-(4-methoxy-1H-pyrrolo[2,3-b]pyridin-3-yl)-N-(1,1,1-trifluoropropan-2-yl)pyrazolo[1,5-a]pyridine-3-carboxamide [127];
5-(4-methoxy-1H-pyrrolo[2,3-b]pyridin-3-yl)-N-(1-methylpiperidin-4-yl)pyrazolo[1,5-a]pyridine-3-carboxamide [128];
5-(4-methoxy-1H-pyrrolo[2,3-b]pyridin-3-yl)-N-(pyridin-3-yl)pyrazolo[1,5-a]pyridine-3-carboxamide [129];
(5-(4-methoxy-1H-pyrrolo[2,3-b]pyridin-3-yl)pyrazolo[1,5-a]pyridin-3-yl)(piperidin-1-yl)methanone [130];
(5-(4-methoxy-1H-pyrrolo[2,3-b]pyridin-3-yl)pyrazolo[1,5-a]pyridin-3-yl)(4-methylpiperazin-1-yl)methanone [131];
(5-(4-methoxy-1H-pyrrolo[2,3-b]pyridin-3-yl)pyrazolo[1,5-a]pyridin-3-yl)(morpholino)methanone [132];
6-(4-methoxy-1H-pyrrolo[2,3-b]pyridin-3-yl)spiro[indene-1,4′-piperidin]-3(2H)-one [134];
6-(4-methoxy-1H-pyrrolo[2,3-b]pyridin-3-yl)-1′-methylspiro[indene-1,4′-piperidin]-3(2H)-one [135];
5-(4-methoxy-1H-pyrrolo[2,3-b]pyridin-3-yl)isoindolin-1-one [136];
5-(4-methoxy-1H-pyrrolo[2,3-b]pyridin-3-yl)-3,3-dimethylisoindolin-1-one [137];
6′-(4-methoxy-1H-pyrrolo[2,3-b]pyridin-3-yl)spiro[cyclohexane-1,1′-isoindolin]-3′-one [138];
3-(chroman-6-yl)-4-methoxy-1H-pyrrolo[2,3-b]pyridine [139];
6-(4-methoxy-1H-pyrrolo[2,3-b]pyridin-3-yl)-3,4-dihydroisoquinolin-1(2H)-one [140];
6-(4-methoxy-1H-pyrrolo[2,3-b]pyridin-3-yl)-4,4-dimethyl-3,4-dihydroisoquinolin-1(2H)-one [141];
7-(4-methoxy-1H-pyrrolo[2,3-b]pyridin-3-yl)spiro[chromane-2,4′-piperidin]-4-one [142];
8-(4-methoxy-1H-pyrrolo[2,3-b]pyridin-3-yl)-3,4-dihydrobenzo[f][1,4]oxazepin-5(2H)-one [143];
6-(4-methoxy-1H-pyrrolo[2,3-b]pyridin-3-yl)quinoline [144];
6-(4-methoxy-1H-pyrrolo[2,3-b]pyridin-3-yl)quinoxaline [145];
6-(4-methoxy-1H-pyrrolo[2,3-b]pyridin-3-yl)quinazoline [146];
4-methoxy-6-(4-methoxy-1H-pyrrolo[2,3-b]pyridin-3-yl)quinazoline [147];
6-(4-methoxy-1H-pyrrolo[2,3-b]pyridin-3-yl)-4-(4-methylpiperazin-1-yl)quinazoline [148];
6-(4-methoxy-1H-pyrrolo[2,3-b]pyridin-3-yl)-4-((1-methylpiperidin-4-yl)oxy)quinazoline [149];
6-(4-methoxy-1H-pyrrolo[2,3-b]pyridin-3-yl)-N-(1-methylpiperidin-4-yl)quinazolin-4-amine [150];
2-methyl-5-(4-(1-methyl-1H-pyrazol-4-yl)-1H-pyrrolo[2,3-b]pyridin-3-yl)-2H-indazole [151];
2-(4-(1-methyl-1H-pyrazol-4-yl)-1H-pyrrolo[2,3-b]pyridin-3-yl)thieno[2,3-c]pyridine [152];
7-(4-(1-methyl-1H-pyrazol-4-yl)-1H-pyrrolo[2,3-b]pyridin-3-yl)-3,4-dihydropyrrolo[1,2-a]pyrazin-1(2H)-one [153];
6-(4-(1-methyl-1H-pyrazol-4-yl)-1H-pyrrolo[2,3-b]pyridin-3-yl)imidazo[1,2-a]pyridine [154];
N-(2,2-difluoroethyl)-6-(4-(1-methyl-1H-pyrazol-4-yl)-1H-pyrrolo[2,3-b]pyridin-3-yl)imidazo[1,2-a]pyridine-3-carboxamide [155];
6-(4-(1-methyl-1H-pyrazol-4-yl)-1H-pyrrolo[2,3-b]pyridin-3-yl)imidazo[1,2-a]pyrimidine [156];
4-fluoro-1-isopropyl-2-methyl-6-(4-(1-methyl-1H-pyrazol-4-yl)-1H-pyrrolo[2,3-b]pyridin-3-yl)-1H-benzo[d]imidazole [157];
4-fluoro-2-methyl-6-(4-(1-methyl-1H-pyrazol-4-yl)-1H-pyrrolo[2,3-b]pyridin-3-yl)-1-(1-methylpiperidin-4-yl)-1H-benzo[d]imidazole [158];
1-isopropyl-2-methyl-6-(4-(1-methyl-1H-pyrazol-4-yl)-1H-pyrrolo[2,3-b]pyridin-3-yl)-1H-imidazo[4,5-b]pyridine [159];
1-isopropyl-2-methyl-6-(4-(1-methyl-1H-pyrazol-4-yl)-1H-pyrrolo[2,3-b]pyridin-3-yl)-1H-imidazo[4,5-c]pyridine [160];
6-(4-(1-methyl-1H-pyrazol-4-yl)-1H-pyrrolo[2,3-b]pyridin-3-yl)-[1,2,4]triazolo[1,5-a]pyridine [161];
7-(4-(1-methyl-1H-pyrazol-4-yl)-1H-pyrrolo[2,3-b]pyridin-3-yl)-[1,2,4]triazolo[1,5-a]pyridine [162];
1-methyl-6-(4-(1-methyl-1H-pyrazol-4-yl)-1H-pyrrolo[2,3-b]pyridin-3-yl)-1H-benzo[d][1,2,3]triazole [163];
6-(4-(1-methyl-1H-pyrazol-4-yl)-1H-pyrrolo[2,3-b]pyridin-3-yl)imidazo[1,2-b]pyridazine [164];
4-(1-methyl-1H-pyrazol-4-yl)-3-(pyrazolo[1,5-a]pyridin-5-yl)-1H-pyrrolo[2,3-b]pyridine [165];
4-(1-methyl-1H-pyrazol-4-yl)-3-(3-(1-methyl-1H-pyrazol-4-yl)pyrazolo[1,5-a]pyridin-5-yl)-1H-pyrrolo[2,3-b]pyridine [166];
4-(1-methyl-1H-pyrazol-4-yl)-3-(3-(pyridin-3-yl)pyrazolo[1,5-a]pyridin-5-yl)-1H-pyrrolo[2,3-b]pyridine [167];
N-(2,2-difluoroethyl)-5-(4-(1-methyl-1H-pyrazol-4-yl)-1H-pyrrolo[2,3-b]pyridin-3-yl)pyrazolo[1,5-a]pyridine-3-carboxamide [168];
(R)-5-(4-(1-methyl-1H-pyrazol-4-yl)-1H-pyrrolo[2,3-b]pyridin-3-yl)-N-(1,1,1-trifluoropropan-2-yl)pyrazolo[1,5-a]pyridine-3-carboxamide [169];
5-(4-(1-methyl-1H-pyrazol-4-yl)-1H-pyrrolo[2,3-b]pyridin-3-yl)-N-(1-methylpiperidin-4-yl)pyrazolo[1,5-a]pyridine-3-carboxamide [170];
5-(4-(1-methyl-1H-pyrazol-4-yl)-1H-pyrrolo[2,3-b]pyridin-3-yl)-N-(pyridin-3-yl)pyrazolo[1,5-a]pyridine-3-carboxamide [171];
(5-(4-(1-methyl-1H-pyrazol-4-yl)-1H-pyrrolo[2,3-b]pyridin-3-yl)pyrazolo[1,5-a]pyridin-3-yl)(piperidin-1-yl)methanone [172];
(5-(4-(1-methyl-1H-pyrazol-4-yl)-1H-pyrrolo[2,3-b]pyridin-3-yl)pyrazolo[1,5-a]pyridin-3-yl)(4-methylpiperazin-1-yl)methanone [173];
(5-(4-(1-methyl-1H-pyrazol-4-yl)-1H-pyrrolo[2,3-b]pyridin-3-yl)pyrazolo[1,5-a]pyridin-3-yl)(morpholino)methanone [174];
6-(4-(1-methyl-1H-pyrazol-4-yl)-1H-pyrrolo[2,3-b]pyridin-3-yl)spiro[indene-1,4′-piperidin]-3(2H)-one [176];
1′-methyl-6-(4-(1-methyl-1H-pyrazol-4-yl)-1H-pyrrolo[2,3-b]pyridin-3-yl)spiro[indene-1,4′-piperidin]-3(2H)-one [177];
5-(4-(1-methyl-1H-pyrazol-4-yl)-1H-pyrrolo[2,3-b]pyridin-3-yl)isoindolin-1-one [178];
3,3-dimethyl-5-(4-(1-methyl-1H-pyrazol-4-yl)-1H-pyrrolo[2,3-b]pyridin-3-yl)isoindolin-1-one [179];
6′-(4-(1-methyl-1H-pyrazol-4-yl)-1H-pyrrolo[2,3-b]pyridin-3-yl)spiro[cyclohexane-1,1′-isoindolin]-3′-one [180];
3-(chroman-6-yl)-4-(1-methyl-1H-pyrazol-4-yl)-1H-pyrrolo[2,3-b]pyridine [181];
6-(4-(1-methyl-1H-pyrazol-4-yl)-1H-pyrrolo[2,3-b]pyridin-3-yl)-3,4-dihydroisoquinolin-1(2H)-one [182];
4,4-dimethyl-6-(4-(1-methyl-1H-pyrazol-4-yl)-1H-pyrrolo[2,3-b]pyridin-3-yl)-3,4-dihydroisoquinolin-1(2H)-one [183];
7-(4-(1-methyl-1H-pyrazol-4-yl)-1H-pyrrolo[2,3-b]pyridin-3-yl)spiro[chromane-2,4′-piperidin]-4-one [184];
8-(4-(1-methyl-1H-pyrazol-4-yl)-1H-pyrrolo[2,3-b]pyridin-3-yl)-3,4-dihydrobenzo[1,4]oxazepin-5(2H)-one [185];
6-(4-(1-methyl-1H-pyrazol-4-yl)-1H-pyrrolo[2,3-b]pyridin-3-yl)quinoline [186];
6-(4-(1-methyl-1H-pyrazol-4-yl)-1H-pyrrolo[2,3-b]pyridin-3-yl)quinoxaline [187];
6-(4-(1-methyl-1H-pyrazol-4-yl)-1H-pyrrolo[2,3-b]pyridin-3-yl)quinazoline [188];
4-methoxy-6-(4-(1-methyl-1H-pyrazol-4-yl)-1H-pyrrolo[2,3-b]pyridin-3-yl)quinazoline [189];
6-(4-(1-methyl-1H-pyrazol-4-yl)-1H-pyrrolo[2,3-b]pyridin-3-yl)-4-(4-methylpiperazin-1-yl)quinazoline [190];
6-(4-(1-methyl-1H-pyrazol-4-yl)-1H-pyrrolo[2,3-b]pyridin-3-yl)-4-((1-methylpiperidin-4-yl)oxy)quinazoline [191];
6-(4-(1-methyl-1H-pyrazol-4-yl)-1H-pyrrolo[2,3-b]pyridin-3-yl)-N-(1-methylpiperidin-4-yl)quinazolin-4-amine [192];
5-(6-chloro-1H-pyrrolo[2,3-b]pyridin-3-yl)-2-methyl-2H-indazole [1411];
2-(6-chloro-1H-pyrrolo[2,3-b]pyridin-3-yl)thieno[2,3-c]pyridine [1412];
7-(6-chloro-1H-pyrrolo[2,3-b]pyridin-3-yl)-3,4-dihydropyrrolo[1,2-a]pyrazin-1(2H)-one [1413];
6-(6-chloro-1H-pyrrolo[2,3-b]pyridin-3-yl)imidazo[1,2-a]pyridine [1414];
6-(6-chloro-1H-pyrrolo[2,3-b]pyridin-3-yl)-N-(2,2-difluoroethyl)imidazo[1,2-a]pyridine-3-carboxamide [1415];
6-(6-chloro-1H-pyrrolo[2,3-b]pyridin-3-yl)imidazo[1,2-a]pyrimidine [1416];
6-(6-chloro-1H-pyrrolo[2,3-b]pyridin-3-yl)-4-fluoro-1-isopropyl-2-methyl-1H-benzo[d]imidazole [1417];
6-(6-chloro-1H-pyrrolo[2,3-b]pyridin-3-yl)-4-fluoro-2-methyl-1-(1-methylpiperidin-4-yl)-1H-benzo[d]imidazole [1418];
6-(6-chloro-1H-pyrrolo[2,3-b]pyridin-3-yl)-1-isopropyl-2-methyl-1H-imidazo[4,5-b]pyridine [1419];
6-(6-chloro-1H-pyrrolo[2,3-b]pyridin-3-yl)-1-isopropyl-2-methyl-1H-imidazo[4,5-c]pyridine [1420];
6-(6-chloro-1H-pyrrolo[2,3-b]pyridin-3-yl)-[1,2,4]triazolo[1,5-a]pyridine [1421];
7-(6-chloro-1H-pyrrolo[2,3-b]pyridin-3-yl)-[1,2,4]triazolo[1,5-a]pyridine [1422];
6-(6-chloro-1H-pyrrolo[2,3-b]pyridin-3-yl)-1-methyl-1H-benzo[d][1,2,3]triazole [1423];
6-(6-chloro-1H-pyrrolo[2,3-b]pyridin-3-yl)imidazo[1,2-b]pyridazine [1424];
6-chloro-3-(pyrazolo[1,5-a]pyridin-5-yl)-1H-pyrrolo[2,3-b]pyridine [1425];
6-chloro-3-(3-(1-methyl-1H-pyrazol-4-yl)pyrazolo[1,5-a]pyridin-5-yl)-1H-pyrrolo[2,3-b]pyridine [1426];
6-chloro-3-(3-(pyridin-3-yl)pyrazolo[1,5-a]pyridin-5-yl)-1H-pyrrolo[2,3-b]pyridine [1427];
5-(6-chloro-1H-pyrrolo[2,3-b]pyridin-3-yl)-N-(2,2-difluoroethyl)pyrazolo[1,5-a]pyridine-3-carboxamide [1428];
(R)-5-(6-chloro-1H-pyrrolo[2,3-b]pyridin-3-yl)-N-(1,1,1-trifluoropropan-2-yl)pyrazolo[1,5-a]pyridine-3-carboxamide [1429];
5-(6-chloro-1H-pyrrolo[2,3-b]pyridin-3-yl)-N-(1-methylpiperidin-4-yl)pyrazolo[1,5-a]pyridine-3-carboxamide [1430];
5-(6-chloro-1H-pyrrolo[2,3-b]pyridin-3-yl)-N-(pyridin-3-yl)pyrazolo[1,5-a]pyridine-3-carboxamide [1431];
(5-(6-chloro-1H-pyrrolo[2,3-b]pyridin-3-yl)pyrazolo[1,5-a]pyridin-3-yl)(piperidin-1-yl)methanone [1432];
(5-(6-chloro-1H-pyrrolo[2,3-b]pyridin-3-yl)pyrazolo[1,5-a]pyridin-3-yl)(4-methylpiperazin-1-yl)methanone [1433];
(5-(6-chloro-1H-pyrrolo[2,3-b]pyridin-3-yl)pyrazolo[1,5-a]pyridin-3-yl)(morpholino)methanone [1434];
6-(6-chloro-1H-pyrrolo[2,3-b]pyridin-3-yl)spiro[indene-1,4′-piperidin]-3(2H)-one [1436];
6-(6-chloro-1H-pyrrolo[2,3-b]pyridin-3-yl)-1′-methylspiro[indene-1,4′-piperidin]-3(2H)-one [1437];
5-(6-chloro-1H-pyrrolo[2,3-b]pyridin-3-yl)isoindolin-1-one [1438];
5-(6-chloro-1H-pyrrolo[2,3-b]pyridin-3-yl)-3,3-dimethylisoindolin-1-one [1439];
6′-(6-chloro-1H-pyrrolo[2,3-b]pyridin-3-yl)spiro[cyclohexane-1,1′-isoindolin]-3′-one [1440];
6-chloro-3-(chroman-6-yl)-1H-pyrrolo[2,3-b]pyridine [1441];
6-(6-chloro-1H-pyrrolo[2,3-b]pyridin-3-yl)-3,4-dihydroisoquinolin-1(2H)-one [1442];
6-(6-chloro-1H-pyrrolo[2,3-b]pyridin-3-yl)-4,4-dimethyl-3,4-dihydroisoquinolin-1(2H)-one [1443];
7-(6-chloro-1H-pyrrolo[2,3-b]pyridin-3-yl)spiro[chromane-2,4′-piperidin]-4-one [1444];
8-(6-chloro-1H-pyrrolo[2,3-b]pyridin-3-yl)-3,4-dihydrobenzo[f][1,4]oxazepin-5(2H)-one [1445];
6-(6-chloro-1H-pyrrolo[2,3-b]pyridin-3-yl)quinoline [1446];
6-(6-chloro-1H-pyrrolo[2,3-b]pyridin-3-yl)quinoxaline [1447];
6-(6-chloro-1H-pyrrolo[2,3-b]pyridin-3-yl)quinazoline [1448];
6-(6-chloro-1H-pyrrolo[2,3-b]pyridin-3-yl)-4-methoxyquinazoline [1449];
6-(6-chloro-1H-pyrrolo[2,3-b]pyridin-3-yl)-4-(4-methylpiperazin-1-yl)quinazoline [1450];
6-(6-chloro-1H-pyrrolo[2,3-b]pyridin-3-yl)-4-((1-methylpiperidin-4-yl)oxy)quinazoline [1451];
6-(6-chloro-1H-pyrrolo[2,3-b]pyridin-3-yl)-N-(1-methylpiperidin-4-yl)quinazolin-4-amine [1452];
2-methyl-5-(6-methyl-1H-pyrrolo[2,3-b]pyridin-3-yl)-2H-indazole [1453];
2-(6-methyl-1H-pyrrolo[2,3-b]pyridin-3-yl)thieno[2,3-c]pyridine [1454];
7-(6-methyl-1H-pyrrolo[2,3-b]pyridin-3-yl)-3,4-dihydropyrrolo[1,2-a]pyrazin-1(2H)-one [1455];
6-(6-methyl-1H-pyrrolo[2,3-b]pyridin-3-yl)imidazo[1,2-a]pyridine [1456];
N-(2,2-difluoroethyl)-6-(6-methyl-1H-pyrrolo[2,3-b]pyridin-3-yl)imidazo[1,2-a]pyridine-3-carboxamide [1457];
6-(6-methyl-1H-pyrrolo[2,3-b]pyridin-3-yl)imidazo[1,2-a]pyrimidine [1458];
4-fluoro-1-isopropyl-2-methyl-6-(6-methyl-1H-pyrrolo[2,3-b]pyridin-3-yl)-1H-benzo[d]imidazole [1459];
4-fluoro-2-methyl-6-(6-methyl-1H-pyrrolo[2,3-b]pyridin-3-yl)-1-(1-methylpiperidin-4-yl)-1H-benzo[d]imidazole [1460];
1-isopropyl-2-methyl-6-(6-methyl-1H-pyrrolo[2,3-b]pyridin-3-yl)-1H-imidazo[4,5-b]pyridine [1461];
1-isopropyl-2-methyl-6-(6-methyl-1H-pyrrolo[2,3-b]pyridin-3-yl)-1H-imidazo[4,5-c]pyridine [1462];
6-(6-methyl-1H-pyrrolo[2,3-b]pyridin-3-yl)-[1,2,4]triazolo[1,5-a]pyridine [1463];
7-(6-methyl-1H-pyrrolo[2,3-b]pyridin-3-yl)-[1,2,4]triazolo[1,5-a]pyridine [1464];
1-methyl-6-(6-methyl-1H-pyrrolo[2,3-b]pyridin-3-yl)-1H-benzo[d][1,2,3]triazole [1465];
6-(6-methyl-1H-pyrrolo[2,3-b]pyridin-3-yl)imidazo[1,2-b]pyridazine [1466];
6-methyl-3-(pyrazolo[1,5-a]pyridin-5-yl)-1H-pyrrolo[2,3-b]pyridine [1467];
6-methyl-3-(3-(1-methyl-1H-pyrazol-4-yl)pyrazolo[1,5-a]pyridin-5-yl)-1H-pyrrolo[2,3-b]pyridine [1468];
6-methyl-3-(3-(pyridin-3-yl)pyrazolo[1,5-a]pyridin-5-yl)-1H-pyrrolo[2,3-b]pyridine [1469];
N-(2,2-difluoroethyl)-5-(6-methyl-1H-pyrrolo[2,3-b]pyridin-3-yl)pyrazolo[1,5-a]pyridine-3-carboxamide [1470];
(R)-5-(6-methyl-1H-pyrrolo[2,3-b]pyridin-3-yl)-N-(1,1,1-trifluoropropan-2-yl)pyrazolo[1,5-a]pyridine-3-carboxamide [1471];
5-(6-methyl-1H-pyrrolo[2,3-b]pyridin-3-yl)-N-(1-methylpiperidin-4-yl)pyrazolo[1,5-a]pyridine-3-carboxamide [1472];
5-(6-methyl-1H-pyrrolo[2,3-b]pyridin-3-yl)-N-(pyridin-3-yl)pyrazolo[1,5-a]pyridine-3-carboxamide [1473];
(5-(6-methyl-1H-pyrrolo[2,3-b]pyridin-3-yl)pyrazolo[1,5-a]pyridin-3-yl)(piperidin-1-yl)methanone [1474];
(5-(6-methyl-1H-pyrrolo[2,3-b]pyridin-3-yl)pyrazolo[1,5-a]pyridin-3-yl)(4-methylpiperazin-1-yl)methanone [1475];
(5-(6-methyl-1H-pyrrolo[2,3-b]pyridin-3-yl)pyrazolo[1,5-a]pyridin-3-yl)(morpholino)methanone [1476];
6-(6-methyl-1H-pyrrolo[2,3-b]pyridin-3-yl)spiro[indene-1,4′-piperidin]-3(2H)-one [1478];
1′-methyl-6-(6-methyl-1H-pyrrolo[2,3-b]pyridin-3-yl)spiro[indene-1,4′-piperidin]-3(2H)-one [1479];
5-(6-methyl-1H-pyrrolo[2,3-b]pyridin-3-yl)isoindolin-1-one [1480];
3,3-dimethyl-5-(6-methyl-1H-pyrrolo[2,3-b]pyridin-3-yl)isoindolin-1-one [1481];
6′-(6-methyl-1H-pyrrolo[2,3-b]pyridin-3-yl)spiro[cyclohexane-1,1′-isoindolin]-3′-one [1482];
3-(chroman-6-yl)-6-methyl-1H-pyrrolo[2,3-b]pyridine [1483];
6-(6-methyl-1H-pyrrolo[2,3-b]pyridin-3-yl)-3,4-dihydroisoquinolin-1(2H)-one [1484];
4,4-dimethyl-6-(6-methyl-1H-pyrrolo[2,3-b]pyridin-3-yl)-3,4-dihydroisoquinolin-1(2H)-one [1485];
7-(6-methyl-1H-pyrrolo[2,3-b]pyridin-3-yl)spiro[chromane-2,4′-piperidin]-4-one [1486];
8-(6-methyl-1H-pyrrolo[2,3-b]pyridin-3-yl)-3,4-dihydrobenzo[f][1,4]oxazepin-5(2H)-one [1487];
6-(6-methyl-1H-pyrrolo[2,3-b]pyridin-3-yl)quinoline [1488];
6-(6-methyl-1H-pyrrolo[2,3-b]pyridin-3-yl)quinoxaline [1489];
6-(6-methyl-1H-pyrrolo[2,3-b]pyridin-3-yl)quinazoline [1490];
4-methoxy-6-(6-methyl-1H-pyrrolo[2,3-b]pyridin-3-yl)quinazoline [1491];
6-(6-methyl-1H-pyrrolo[2,3-b]pyridin-3-yl)-4-(4-methylpiperazin-1-yl)quinazoline [1492];
6-(6-methyl-1H-pyrrolo[2,3-b]pyridin-3-yl)-4-((1-methylpiperidin-4-yl)oxy)quinazoline [1493];
6-(6-methyl-1H-pyrrolo[2,3-b]pyridin-3-yl)-N-(1-methylpiperidin-4-yl)quinazolin-4-amine [1494];
5-(6-cyclopropyl-1H-pyrrolo[2,3-b]pyridin-3-yl)-2-methyl-2H-indazole [1495];
2-(6-cyclopropyl-1H-pyrrolo[2,3-b]pyridin-3-yl)thieno[2,3-c]pyridine [1496];
7-(6-cyclopropyl-1H-pyrrolo[2,3-b]pyridin-3-yl)-3,4-dihydropyrrolo[1,2-a]pyrazin-1(2H)-one [1497];
6-(6-cyclopropyl-1H-pyrrolo[2,3-b]pyridin-3-yl)imidazo[1,2-a]pyridine [1498];
6-(6-cyclopropyl-1H-pyrrolo[2,3-b]pyridin-3-yl)-N-(2,2-difluoroethyl)imidazo[1,2-a]pyridine-3-carboxamide [1499];
6-(6-cyclopropyl-1H-pyrrolo[2,3-b]pyridin-3-yl)imidazo[1,2-a]pyrimidine [1500];
6-(6-cyclopropyl-1H-pyrrolo[2,3-b]pyridin-3-yl)-4-fluoro-1-isopropyl-2-methyl-1H-benzo[d]imidazole [1501];
6-(6-cyclopropyl-1H-pyrrolo[2,3-b]pyridin-3-yl)-4-fluoro-2-methyl-1-(1-methylpiperidin-4-yl)-1H-benzo[d]imidazole [1502];
6-(6-cyclopropyl-1H-pyrrolo[2,3-b]pyridin-3-yl)-1-isopropyl-2-methyl-1H-imidazo[4,5-b]pyridine [1503];
6-(6-cyclopropyl-1H-pyrrolo[2,3-b]pyridin-3-yl)-1-isopropyl-2-methyl-1H-imidazo[4,5-c]pyridine [1504];
6-(6-cyclopropyl-1H-pyrrolo[2,3-b]pyridin-3-yl)-[1,2,4]triazolo[1,5-a]pyridine [1505];
7-(6-cyclopropyl-1H-pyrrolo[2,3-b]pyridin-3-yl)-[1,2,4]triazolo[1,5-a]pyridine [1506];
6-(6-cyclopropyl-1H-pyrrolo[2,3-b]pyridin-3-yl)-1-methyl-1H-benzo[d][1,2,3]triazole [1507];
6-(6-cyclopropyl-1H-pyrrolo[2,3-b]pyridin-3-yl)imidazo[1,2-b]pyridazine [1508];
6-cyclopropyl-3-(pyrazolo[1,5-a]pyridin-5-yl)-1H-pyrrolo[2,3-b]pyridine [1509];
6-cyclopropyl-3-(3-(1-methyl-1H-pyrazol-4-yl)pyrazolo[1,5-a]pyridin-5-yl)-1H-pyrrolo[2,3-b]pyridine [1510];
6-cyclopropyl-3-(3-(pyridin-3-yl)pyrazolo[1,5-a]pyridin-5-yl)-1H-pyrrolo[2,3-b]pyridine [1511];
5-(6-cyclopropyl-1H-pyrrolo[2,3-b]pyridin-3-yl)-N-(2,2-difluoroethyl)pyrazolo[1,5-a]pyridine-3-carboxamide [1512];
(R)-5-(6-cyclopropyl-1H-pyrrolo[2,3-b]pyridin-3-yl)-N-(1,1,1-trifluoropropan-2-yl)pyrazolo[1,5-a]pyridine-3-carboxamide [1513];
5-(6-cyclopropyl-1H-pyrrolo[2,3-b]pyridin-3-yl)-N-(1-methylpiperidin-4-yl)pyrazolo[1,5-a]pyridine-3-carboxamide [1514];
5-(6-cyclopropyl-1H-pyrrolo[2,3-b]pyridin-3-yl)-N-(pyridin-3-yl)pyrazolo[1,5-a]pyridine-3-carboxamide [1515];
(5-(6-cyclopropyl-1H-pyrrolo[2,3-b]pyridin-3-yl)pyrazolo[1,5-a]pyridin-3-yl)(piperidin-1-yl)methanone [1516];
(5-(6-cyclopropyl-1H-pyrrolo[2,3-b]pyridin-3-yl)pyrazolo[1,5-a]pyridin-3-yl)(4-methylpiperazin-1-yl)methanone [1517];
(5-(6-cyclopropyl-1H-pyrrolo[2,3-b]pyridin-3-yl)pyrazolo[1,5-a]pyridin-3-yl)(morpholino)methanone [1518];
6-cyclopropyl-3-(2-(4-methylpiperazin-1-yl)pyridin-4-yl)-1H-pyrrolo[2,3-b]pyridine [1519];
6-(6-cyclopropyl-1H-pyrrolo[2,3-b]pyridin-3-yl)spiro[indene-1,4′-piperidin]-3(2H)-one [1520];
6-(6-cyclopropyl-1H-pyrrolo[2,3-b]pyridin-3-yl)-1′-methylspiro[indene-1,4′-piperidin]-3(2H)-one [1521];
5-(6-cyclopropyl-1H-pyrrolo[2,3-b]pyridin-3-yl)-3,3-dimethylisoindolin-1-one [1523];
6′-(6-cyclopropyl-1H-pyrrolo[2,3-b]pyridin-3-yl)spiro[cyclohexane-1,1′-isoindolin]-3′-one [1524];
3-(chroman-6-yl)-6-cyclopropyl-1H-pyrrolo[2,3-b]pyridine [1525];
6-(6-cyclopropyl-1H-pyrrolo[2,3-b]pyridin-3-yl)-3,4-dihydroisoquinolin-1(2H)-one [1526];
6-(6-cyclopropyl-1H-pyrrolo[2,3-b]pyridin-3-yl)-4,4-dimethyl-3,4-dihydroisoquinolin-1(2H)-one [1527];
7-(6-cyclopropyl-1H-pyrrolo[2,3-b]pyridin-3-yl)spiro[chromane-2,4′-piperidin]-4-one [1528];
8-(6-cyclopropyl-1H-pyrrolo[2,3-b]pyridin-3-yl)-3,4-dihydrobenzo[f][1,4]oxazepin-5(2H)-one [1529];
6-(6-cyclopropyl-1H-pyrrolo[2,3-b]pyridin-3-yl)quinoline [1530];
6-(6-cyclopropyl-1H-pyrrolo[2,3-b]pyridin-3-yl)quinoxaline [1531];
6-(6-cyclopropyl-1H-pyrrolo[2,3-b]pyridin-3-yl)quinazoline [1532];
6-(6-cyclopropyl-1H-pyrrolo[2,3-b]pyridin-3-yl)-4-methoxyquinazoline [1533];
6-(6-cyclopropyl-1H-pyrrolo[2,3-b]pyridin-3-yl)-4-(4-methylpiperazin-1-yl)quinazoline [1534];
6-(6-cyclopropyl-1H-pyrrolo[2,3-b]pyridin-3-yl)-4-((1-methylpiperidin-4-yl)oxy)quinazoline [1535];
6-(6-cyclopropyl-1H-pyrrolo[2,3-b]pyridin-3-yl)-N-(1-methylpiperidin-4-yl)quinazolin-4-amine [1536];
5-(6-cyclobutyl-1H-pyrrolo[2,3-b]pyridin-3-yl)-2-methyl-2H-indazole [1537];
2-(6-cyclobutyl-1H-pyrrolo[2,3-b]pyridin-3-yl)thieno[2,3-c]pyridine [1538];
7-(6-cyclobutyl-1H-pyrrolo[2,3-b]pyridin-3-yl)-3,4-dihydropyrrolo[1,2-a]pyrazin-1(2H)-one [1539];
6-(6-cyclobutyl-1H-pyrrolo[2,3-b]pyridin-3-yl)imidazo[1,2-a]pyridine [1540];
6-(6-cyclobutyl-1H-pyrrolo[2,3-b]pyridin-3-yl)-N-(2,2-difluoroethyl)imidazo[1,2-a]pyridine-3-carboxamide [1541];
6-(6-cyclobutyl-1H-pyrrolo[2,3-b]pyridin-3-yl)imidazo[1,2-a]pyrimidine [1542];
6-(6-cyclobutyl-1H-pyrrolo[2,3-b]pyridin-3-yl)-4-fluoro-1-isopropyl-2-methyl-1H-benzo[d]imidazole [1543];
6-(6-cyclobutyl-1H-pyrrolo[2,3-b]pyridin-3-yl)-4-fluoro-2-methyl-1-(1-methylpiperidin-4-yl)-1H-benzo[d]imidazole [1544];
6-(6-cyclobutyl-1H-pyrrolo[2,3-b]pyridin-3-yl)-1-isopropyl-2-methyl-1H-imidazo[4,5-b]pyridine [1545];
6-(6-cyclobutyl-1H-pyrrolo[2,3-b]pyridin-3-yl)-1-isopropyl-2-methyl-1H-imidazo[4,5-c]pyridine [1546];
6-(6-cyclobutyl-1H-pyrrolo[2,3-b]pyridin-3-yl)-[1,2,4]triazolo[1,5-a]pyridine [1547];
7-(6-cyclobutyl-1H-pyrrolo[2,3-b]pyridin-3-yl)-[1,2,4]triazolo[1,5-a]pyridine [1548];
6-(6-cyclobutyl-1H-pyrrolo[2,3-b]pyridin-3-yl)-1-methyl-1H-benzo[d][1,2,3]triazole [1549];
6-(6-cyclobutyl-1H-pyrrolo[2,3-b]pyridin-3-yl)imidazo[1,2-b]pyridazine [1550];
6-cyclobutyl-3-(pyrazolo[1,5-a]pyridin-5-yl)-1H-pyrrolo[2,3-b]pyridine [1551];
6-cyclobutyl-3-(3-(1-methyl-1H-pyrazol-4-yl)pyrazolo[1,5-a]pyridin-5-yl)-1H-pyrrolo[2,3-b]pyridine [1552];
6-cyclobutyl-3-(3-(pyridin-3-yl)pyrazolo[1,5-a]pyridin-5-yl)-1H-pyrrolo[2,3-b]pyridine [1553];
5-(6-cyclobutyl-1H-pyrrolo[2,3-b]pyridin-3-yl)-N-(2,2-difluoroethyl)pyrazolo[1,5-a]pyridine-3-carboxamide [1554];
(R)-5-(6-cyclobutyl-1H-pyrrolo[2,3-b]pyridin-3-yl)-N-(1,1,1-trifluoropropan-2-yl)pyrazolo[1,5-a]pyridine-3-carboxamide [1555];
5-(6-cyclobutyl-1H-pyrrolo[2,3-b]pyridin-3-yl)-N-(1-methylpiperidin-4-yl)pyrazolo[1,5-a]pyridine-3-carboxamide [1556];
5-(6-cyclobutyl-1H-pyrrolo[2,3-b]pyridin-3-yl)-N-(pyridin-3-yl)pyrazolo[1,5-a]pyridine-3-carboxamide [1557];
(5-(6-cyclobutyl-1H-pyrrolo[2,3-b]pyridin-3-yl)pyrazolo[1,5-a]pyridin-3-yl)(piperidin-1-yl)methanone [1558];
(5-(6-cyclobutyl-1H-pyrrolo[2,3-b]pyridin-3-yl)pyrazolo[1,5-a]pyridin-3-yl)(4-methylpiperazin-1-yl)methanone [1559];
(5-(6-cyclobutyl-1H-pyrrolo[2,3-b]pyridin-3-yl)pyrazolo[1,5-a]pyridin-3-yl)(morpholino)methanone [1560];
6-(6-cyclobutyl-1H-pyrrolo[2,3-b]pyridin-3-yl)spiro[indene-1,4′-piperidin]-3(2H)-one [1562];
6-(6-cyclobutyl-1H-pyrrolo[2,3-b]pyridin-3-yl)-1′-methylspiro[indene-1,4′-piperidin]-3(2H)-one [1563];
5-(6-cyclobutyl-1H-pyrrolo[2,3-b]pyridin-3-yl)isoindolin-1-one [1564];
5-(6-cyclobutyl-1H-pyrrolo[2,3-b]pyridin-3-yl)-3,3-dimethylisoindolin-1-one [1565];
6′-(6-cyclobutyl-1H-pyrrolo[2,3-b]pyridin-3-yl)spiro[cyclohexane-1,1′-isoindolin]-3′-one [1566];
3-(chroman-6-yl)-6-cyclobutyl-1H-pyrrolo[2,3-b]pyridine [1567];
6-(6-cyclobutyl-1H-pyrrolo[2,3-b]pyridin-3-yl)-3,4-dihydroisoquinolin-1(2H)-one [1568];
6-(6-cyclobutyl-1H-pyrrolo[2,3-b]pyridin-3-yl)-4,4-dimethyl-3,4-dihydroisoquinolin-1(2H)-one [1569];
7-(6-cyclobutyl-1H-pyrrolo[2,3-b]pyridin-3-yl)spiro[chromane-2,4′-piperidin]-4-one [1570];
8-(6-cyclobutyl-1H-pyrrolo[2,3-b]pyridin-3-yl)-3,4-dihydrobenzo[f][1,4]oxazepin-5(2H)-one [1571];
6-(6-cyclobutyl-1H-pyrrolo[2,3-b]pyridin-3-yl)quinoline [1572];
6-(6-cyclobutyl-1H-pyrrolo[2,3-b]pyridin-3-yl)quinoxaline [1573];
6-(6-cyclobutyl-1H-pyrrolo[2,3-b]pyridin-3-yl)quinazoline [1574];
6-(6-cyclobutyl-1H-pyrrolo[2,3-b]pyridin-3-yl)-4-methoxyquinazoline [1575];
6-(6-cyclobutyl-1H-pyrrolo[2,3-b]pyridin-3-yl)-4-(4-methylpiperazin-1-yl)quinazoline [1576];
6-(6-cyclobutyl-1H-pyrrolo[2,3-b]pyridin-3-yl)-4-((1-methylpiperidin-4-yl)oxy)quinazoline [1577];
6-(6-cyclobutyl-1H-pyrrolo[2,3-b]pyridin-3-yl)-N-(1-methylpiperidin-4-yl)quinazolin-4-amine [1578];
5-(6-cyclopentyl-1H-pyrrolo[2,3-b]pyridin-3-yl)-2-methyl-2H-indazole [1579];
2-(6-cyclopentyl-1H-pyrrolo[2,3-b]pyridin-3-yl)thieno[2,3-c]pyridine [1580];
7-(6-cyclopentyl-1H-pyrrolo[2,3-b]pyridin-3-yl)-3,4-dihydropyrrolo[1,2-a]pyrazin-1(2H)-one [1581];
6-(6-cyclopentyl-1H-pyrrolo[2,3-b]pyridin-3-yl)imidazo[1,2-a]pyridine [1582];
6-(6-cyclopentyl-1H-pyrrolo[2,3-b]pyridin-3-yl)-N-(2,2-difluoroethyl)imidazo[1,2-a]pyridine-3-carboxamide [1583];
6-(6-cyclopentyl-1H-pyrrolo[2,3-b]pyridin-3-yl)imidazo[1,2-a]pyrimidine [1584];
6-(6-cyclopentyl-1H-pyrrolo[2,3-b]pyridin-3-yl)-4-fluoro-1-isopropyl-2-methyl-1H-benzo[d]imidazole [1585];
6-(6-cyclopentyl-1H-pyrrolo[2,3-b]pyridin-3-yl)-4-fluoro-2-methyl-1-(1-methylpiperidin-4-yl)-1H-benzo[d]imidazole [1586];
6-(6-cyclopentyl-1H-pyrrolo[2,3-b]pyridin-3-yl)-1-isopropyl-2-methyl-1H-imidazo[4,5-b]pyridine [1587];
6-(6-cyclopentyl-1H-pyrrolo[2,3-b]pyridin-3-yl)-1-isopropyl-2-methyl-1H-imidazo[4,5-c]pyridine [1588];
6-(6-cyclopentyl-1H-pyrrolo[2,3-b]pyridin-3-yl)-[1,2,4]triazolo[1,5-a]pyridine [1589];
7-(6-cyclopentyl-1H-pyrrolo[2,3-b]pyridin-3-yl)-[1,2,4]triazolo[1,5-a]pyridine [1590];
6-(6-cyclopentyl-1H-pyrrolo[2,3-b]pyridin-3-yl)-1-methyl-1H-benzo[d][1,2,3]triazole [1591];
6-(6-cyclopentyl-1H-pyrrolo[2,3-b]pyridin-3-yl)imidazo[1,2-b]pyridazine [1592];
6-cyclopentyl-3-(pyrazolo[1,5-a]pyridin-5-yl)-1H-pyrrolo[2,3-b]pyridine [1593];
6-cyclopentyl-3-(3-(1-methyl-1H-pyrazol-4-yl)pyrazolo[1,5-a]pyridin-5-yl)-1H-pyrrolo[2,3-b]pyridine [1594];
6-cyclopentyl-3-(3-(pyridin-3-yl)pyrazolo[1,5-a]pyridin-5-yl)-1H-pyrrolo[2,3-b]pyridine [1595];
5-(6-cyclopentyl-1H-pyrrolo[2,3-b]pyridin-3-yl)-N-(2,2-difluoroethyl)pyrazolo[1,5-a]pyridine-3-carboxamide [1596];
(R)-5-(6-cyclopentyl-1H-pyrrolo[2,3-b]pyridin-3-yl)-N-(1,1,1-trifluoropropan-2-yl)pyrazolo[1,5-a]pyridine-3-carboxamide [1597];
5-(6-cyclopentyl-1H-pyrrolo[2,3-b]pyridin-3-yl)-N-(1-methylpiperidin-4-yl)pyrazolo[1,5-a]pyridine-3-carboxamide [1598];
5-(6-cyclopentyl-1H-pyrrolo[2,3-b]pyridin-3-yl)-N-(pyridin-3-yl)pyrazolo[1,5-a]pyridine-3-carboxamide [1599];
(5-(6-cyclopentyl-1H-pyrrolo[2,3-b]pyridin-3-yl)pyrazolo[1,5-a]pyridin-3-yl)(piperidin-1-yl)methanone [1600];
(5-(6-cyclopentyl-1H-pyrrolo[2,3-b]pyridin-3-yl)pyrazolo[1,5-a]pyridin-3-yl)(4-methylpiperazin-1-yl)methanone [1601];
(5-(6-cyclopentyl-1H-pyrrolo[2,3-b]pyridin-3-yl)pyrazolo[1,5-a]pyridin-3-yl)(morpholino)methanone [1602];
6-(6-cyclopentyl-1H-pyrrolo[2,3-b]pyridin-3-yl)spiro[indene-1,4′-piperidin]-3(2H)-one [1604];
6-(6-cyclopentyl-1H-pyrrolo[2,3-b]pyridin-3-yl)-1′-methylspiro[indene-1,4′-piperidin]-3(2H)-one [1605];
5-(6-cyclopentyl-1H-pyrrolo[2,3-b]pyridin-3-yl)isoindolin-1-one [1606];
5-(6-cyclopentyl-1H-pyrrolo[2,3-b]pyridin-3-yl)-3,3-dimethylisoindolin-1-one [1607];
6′-(6-cyclopentyl-1H-pyrrolo[2,3-b]pyridin-3-yl)spiro[cyclohexane-1,1′-isoindolin]-3′-one [1608];
3-(chroman-6-yl)-6-cyclopentyl-1H-pyrrolo[2,3-b]pyridine [1609];
6-(6-cyclopentyl-1H-pyrrolo[2,3-b]pyridin-3-yl)-3,4-dihydroisoquinolin-1(2H)-one [1610];
6-(6-cyclopentyl-1H-pyrrolo[2,3-b]pyridin-3-yl)-4,4-dimethyl-3,4-dihydroisoquinolin-1(2H)-one [1611];
7-(6-cyclopentyl-1H-pyrrolo[2,3-b]pyridin-3-yl)spiro[chromane-2,4′-piperidin]-4-one [1612];
8-(6-cyclopentyl-1H-pyrrolo[2,3-b]pyridin-3-yl)-3,4-dihydrobenzo[f][1,4]oxazepin-5(2H)-one [1613];
6-(6-cyclopentyl-1H-pyrrolo[2,3-b]pyridin-3-yl)quinoline [1614];
6-(6-cyclopentyl-1H-pyrrolo[2,3-b]pyridin-3-yl)quinoxaline [1615];
6-(6-cyclopentyl-1H-pyrrolo[2,3-b]pyridin-3-yl)quinazoline [1616];
6-(6-cyclopentyl-1H-pyrrolo[2,3-b]pyridin-3-yl)-4-methoxyquinazoline [1617];
6-(6-cyclopentyl-1H-pyrrolo[2,3-b]pyridin-3-yl)-4-(4-methylpiperazin-1-yl)quinazoline [1618];
6-(6-cyclopentyl-1H-pyrrolo[2,3-b]pyridin-3-yl)-4-((1-methylpiperidin-4-yl)oxy)quinazoline [1619];
6-(6-cyclopentyl-1H-pyrrolo[2,3-b]pyridin-3-yl)-N-(1-methylpiperidin-4-yl)quinazolin-4-amine [1620];
5-(6-(cyclobutylmethyl)-1H-pyrrolo[2,3-b]pyridin-3-yl)-2-methyl-2H-indazole [1621];
2-(6-(cyclobutylmethyl)-1H-pyrrolo[2,3-b]pyridin-3-yl)thieno[2,3-c]pyridine [1622];
7-(6-(cyclobutylmethyl)-1H-pyrrolo[2,3-b]pyridin-3-yl)-3,4-dihydropyrrolo[1,2-a]pyrazin-1(2H)-one [1623];
6-(6-(cyclobutylmethyl)-1H-pyrrolo[2,3-b]pyridin-3-yl)imidazo[1,2-a]pyridine [1624];
6-(6-(cyclobutylmethyl)-1H-pyrrolo[2,3-b]pyridin-3-yl)-N-(2,2-difluoroethyl)imidazo[1,2-a]pyridine-3-carboxamide [1625];
6-(6-(cyclobutylmethyl)-1H-pyrrolo[2,3-b]pyridin-3-yl)imidazo[1,2-a]pyrimidine [1626];
6-(6-(cyclobutylmethyl)-1H-pyrrolo[2,3-b]pyridin-3-yl)-4-fluoro-1-isopropyl-2-methyl-1H-benzo[d]imidazole [1627];
6-(6-(cyclobutylmethyl)-1H-pyrrolo[2,3-b]pyridin-3-yl)-4-fluoro-2-methyl-1-(1-methylpiperidin-4-yl)-1H-benzo[d]imidazole [1628];
6-(6-(cyclobutylmethyl)-1H-pyrrolo[2,3-b]pyridin-3-yl)-1-isopropyl-2-methyl-1H-imidazo[4,5-b]pyridine [1629];
6-(6-(cyclobutylmethyl)-1H-pyrrolo[2,3-b]pyridin-3-yl)-1-isopropyl-2-methyl-1H-imidazo[4,5-c]pyridine [1630];
6-(6-(cyclobutylmethyl)-1H-pyrrolo[2,3-b]pyridin-3-yl)-[1,2,4]triazolo[1,5-a]pyridine [1631];
7-(6-(cyclobutylmethyl)-1H-pyrrolo[2,3-b]pyridin-3-yl)-[1,2,4]triazolo[1,5-a]pyridine [1632];
6-(6-(cyclobutylmethyl)-1H-pyrrolo[2,3-b]pyridin-3-yl)-1-methyl-1H-benzo[d][1,2,3]triazole [1633];
6-(6-(cyclobutylmethyl)-1H-pyrrolo[2,3-b]pyridin-3-yl)imidazo[1,2-b]pyridazine [1634];
6-(cyclobutylmethyl)-3-(pyrazolo[1,5-a]pyridin-5-yl)-1H-pyrrolo[2,3-b]pyridine [1635];
6-(cyclobutylmethyl)-3-(3-(1-methyl-1H-pyrazol-4-yl)pyrazolo[1,5-a]pyridin-5-yl)-1H-pyrrolo[2,3-b]pyridine [1636];
6-(cyclobutylmethyl)-3-(3-(pyridin-3-yl)pyrazolo[1,5-a]pyridin-5-yl)-1H-pyrrolo[2,3-b]pyridine [1637];
5-(6-(cyclobutylmethyl)-1H-pyrrolo[2,3-b]pyridin-3-yl)-N-(2,2-difluoroethyl)pyrazolo[1,5-a]pyridine-3-carboxamide [1638];
(R)-5-(6-(cyclobutylmethyl)-1H-pyrrolo[2,3-b]pyridin-3-yl)-N-(1,1,1-trifluoropropan-2-yl)pyrazolo[1,5-a]pyridine-3-carboxamide [1639];
5-(6-(cyclobutylmethyl)-1H-pyrrolo[2,3-b]pyridin-3-yl)-N-(1-methylpiperidin-4-yl)pyrazolo[1,5-a]pyridine-3-carboxamide [1640];
5-(6-(cyclobutylmethyl)-1H-pyrrolo[2,3-b]pyridin-3-yl)-N-(pyridin-3-yl)pyrazolo[1,5-a]pyridine-3-carboxamide [1641];
(5-(6-(cyclobutylmethyl)-1H-pyrrolo[2,3-b]pyridin-3-yl)pyrazolo[1,5-a]pyridin-3-yl)(piperidin-1-yl)methanone [1642];
(5-(6-(cyclobutylmethyl)-1H-pyrrolo[2,3-b]pyridin-3-yl)pyrazolo[1,5-a]pyridin-3-yl)(4-methylpiperazin-1-yl)methanone [1643];
(5-(6-(cyclobutylmethyl)-1H-pyrrolo[2,3-b]pyridin-3-yl)pyrazolo[1,5-a]pyridin-3-yl)(morpholino)methanone [1644];
6-(6-(cyclobutylmethyl)-1H-pyrrolo[2,3-b]pyridin-3-yl)spiro[indene-1,4′-piperidin]-3(2H)-one [1646];
6-(6-(cyclobutylmethyl)-1H-pyrrolo[2,3-b]pyridin-3-yl)-1′-methylspiro[indene-1,4′-piperidin]-3(2H)-one [1647];
5-(6-(cyclobutylmethyl)-1H-pyrrolo[2,3-b]pyridin-3-yl)isoindolin-1-one [1648];
5-(6-(cyclobutylmethyl)-1H-pyrrolo[2,3-b]pyridin-3-yl)-3,3-dimethylisoindolin-1-one [1649];
6′-(6-(cyclobutylmethyl)-1H-pyrrolo[2,3-b]pyridin-3-yl)spiro[cyclohexane-1,1′-isoindolin]-3′-one [1650];
3-(chroman-6-yl)-6-(cyclobutylmethyl)-1H-pyrrolo[2,3-b]pyridine [1651];
6-(6-(cyclobutylmethyl)-1H-pyrrolo[2,3-b]pyridin-3-yl)-3,4-dihydroisoquinolin-1(2H)-one [1652];
6-(6-(cyclobutylmethyl)-1H-pyrrolo[2,3-b]pyridin-3-yl)-4,4-dimethyl-3,4-dihydroisoquinolin-1(2H)-one [1653];
7-(6-(cyclobutylmethyl)-1H-pyrrolo[2,3-b]pyridin-3-yl)spiro[chromane-2,4′-piperidin]-4-one [1654];
8-(6-(cyclobutylmethyl)-1H-pyrrolo[2,3-b]pyridin-3-yl)-3,4-dihydrobenzo[f][1,4]oxazepin-5(2H)-one [1655];
6-(6-(cyclobutylmethyl)-1H-pyrrolo[2,3-b]pyridin-3-yl)quinoline [1656];
6-(6-(cyclobutylmethyl)-1H-pyrrolo[2,3-b]pyridin-3-yl)quinoxaline [1657];
6-(6-(cyclobutylmethyl)-1H-pyrrolo[2,3-b]pyridin-3-yl)quinazoline [1658];
6-(6-(cyclobutylmethyl)-1H-pyrrolo[2,3-b]pyridin-3-yl)-4-methoxyquinazoline [1659];
6-(6-(cyclobutylmethyl)-1H-pyrrolo[2,3-b]pyridin-3-yl)-4-(4-methylpiperazin-1-yl)quinazoline [1660];
6-(6-(cyclobutylmethyl)-1H-pyrrolo[2,3-b]pyridin-3-yl)-4-((1-methylpiperidin-4-yl)oxy)quinazoline [1661];
6-(6-(cyclobutylmethyl)-1H-pyrrolo[2,3-b]pyridin-3-yl)-N-(1-methylpiperidin-4-yl)quinazolin-4-amine [1662];
5-(6-methoxy-1H-pyrrolo[2,3-b]pyridin-3-yl)-2-methyl-2H-indazole [1663];
2-(6-methoxy-1H-pyrrolo[2,3-b]pyridin-3-yl)thieno[2,3-c]pyridine [1664];
7-(6-methoxy-1H-pyrrolo[2,3-b]pyridin-3-yl)-3,4-dihydropyrrolo[1,2-a]pyrazin-1(2H)-one [1665];
6-(6-methoxy-1H-pyrrolo[2,3-b]pyridin-3-yl)imidazo[1,2-a]pyridine [1666];
N-(2,2-difluoroethyl)-6-(6-methoxy-1H-pyrrolo[2,3-b]pyridin-3-yl)imidazo[1,2-a]pyridine-3-carboxamide [1667];
6-(6-methoxy-1H-pyrrolo[2,3-b]pyridin-3-yl)imidazo[1,2-a]pyrimidine [1668];
4-fluoro-1-isopropyl-6-(6-methoxy-1H-pyrrolo[2,3-b]pyridin-3-yl)-2-methyl-1H-benzo[d]imidazole [1669];
4-fluoro-6-(6-methoxy-1H-pyrrolo[2,3-b]pyridin-3-yl)-2-methyl-1-(1-methylpiperidin-4-yl)-1H-benzo[d]imidazole [1670];
1-isopropyl-6-(6-methoxy-1H-pyrrolo[2,3-b]pyridin-3-yl)-2-methyl-1H-imidazo[4,5-b]pyridine [1671];
1-isopropyl-6-(6-methoxy-1H-pyrrolo[2,3-b]pyridin-3-yl)-2-methyl-1H-imidazo[4,5-c]pyridine [1672];
6-(6-methoxy-1H-pyrrolo[2,3-b]pyridin-3-yl)-[1,2,4]triazolo[1,5-a]pyridine [1673];
7-(6-methoxy-1H-pyrrolo[2,3-b]pyridin-3-yl)-[1,2,4]triazolo[1,5-a]pyridine [1674];
6-(6-methoxy-1H-pyrrolo[2,3-b]pyridin-3-yl)-1-methyl-1H-benzo[d][1,2,3]triazole [1675];
6-(6-methoxy-1H-pyrrolo[2,3-b]pyridin-3-yl)imidazo[1,2-b]pyridazine [1676];
6-methoxy-3-(pyrazolo[1,5-a]pyridin-5-yl)-1H-pyrrolo[2,3-b]pyridine [1677];
6-methoxy-3-(3-(1-methyl-1H-pyrazol-4-yl)pyrazolo[1,5-a]pyridin-5-yl)-1H-pyrrolo[2,3-b]pyridine [1678];
6-methoxy-3-(3-(pyridin-3-yl)pyrazolo[1,5-a]pyridin-5-yl)-1H-pyrrolo[2,3-b]pyridine [1679];
N-(2,2-difluoroethyl)-5-(6-methoxy-1H-pyrrolo[2,3-b]pyridin-3-yl)pyrazolo[1,5-a]pyridine-3-carboxamide [1680];
(R)-5-(6-methoxy-1H-pyrrolo[2,3-b]pyridin-3-yl)-N-(1,1,1-trifluoropropan-2-yl)pyrazolo[1,5-a]pyridine-3-carboxamide [1681];
5-(6-methoxy-1H-pyrrolo[2,3-b]pyridin-3-yl)-N-(1-methylpiperidin-4-yl)pyrazolo[1,5-a]pyridine-3-carboxamide [1682];
5-(6-methoxy-1H-pyrrolo[2,3-b]pyridin-3-yl)-N-(pyridin-3-yl)pyrazolo[1,5-a]pyridine-3-carboxamide [1683];
(5-(6-methoxy-1H-pyrrolo[2,3-b]pyridin-3-yl)pyrazolo[1,5-a]pyridin-3-yl)(piperidin-1-yl)methanone [1684];
(5-(6-methoxy-1H-pyrrolo[2,3-b]pyridin-3-yl)pyrazolo[1,5-a]pyridin-3-yl)(4-methylpiperazin-1-yl)methanone [1685];
(5-(6-methoxy-1H-pyrrolo[2,3-b]pyridin-3-yl)pyrazolo[1,5-a]pyridin-3-yl)(morpholino)methanone [1686];
6-(6-methoxy-1H-pyrrolo[2,3-b]pyridin-3-yl)spiro[indene-1,4′-piperidin]-3(2H)-one [1688];
6-(6-methoxy-1H-pyrrolo[2,3-b]pyridin-3-yl)-1′-methylspiro[indene-1,4′-piperidin]-3(2H)-one [1689];
5-(6-methoxy-1H-pyrrolo[2,3-b]pyridin-3-yl)isoindolin-1-one [1690];
5-(6-methoxy-1H-pyrrolo[2,3-b]pyridin-3-yl)-3,3-dimethylisoindolin-1-one [1691];
6′-(6-methoxy-1H-pyrrolo[2,3-b]pyridin-3-yl)spiro[cyclohexane-1,1′-isoindolin]-3′-one [1692];
3-(chroman-6-yl)-6-methoxy-1H-pyrrolo[2,3-b]pyridine [1693];
6-(6-methoxy-1H-pyrrolo[2,3-b]pyridin-3-yl)-3,4-dihydroisoquinolin-1(2H)-one [1694];
6-(6-methoxy-1H-pyrrolo[2,3-b]pyridin-3-yl)-4,4-dimethyl-3,4-dihydroisoquinolin-1(2H)-one [1695];
7-(6-methoxy-1H-pyrrolo[2,3-b]pyridin-3-yl)spiro[chromane-2,4′-piperidin]-4-one [1696];
8-(6-methoxy-1H-pyrrolo[2,3-b]pyridin-3-yl)-3,4-dihydrobenzo[f][1,4]oxazepin-5(2H)-one [1697];
6-(6-methoxy-1H-pyrrolo[2,3-b]pyridin-3-yl)quinoline [1698];
6-(6-methoxy-1H-pyrrolo[2,3-b]pyridin-3-yl)quinoxaline [1699];
6-(6-methoxy-1H-pyrrolo[2,3-b]pyridin-3-yl)quinazoline [1700];
4-methoxy-6-(6-methoxy-1H-pyrrolo[2,3-b]pyridin-3-yl)quinazoline [1701];
6-(6-methoxy-1H-pyrrolo[2,3-b]pyridin-3-yl)-4-(4-methylpiperazin-1-yl)quinazoline [1702];
6-(6-methoxy-1H-pyrrolo[2,3-b]pyridin-3-yl)-4-((1-methylpiperidin-4-yl)oxy)quinazoline [1703];
6-(6-methoxy-1H-pyrrolo[2,3-b]pyridin-3-yl)-N-(1-methylpiperidin-4-yl)quinazolin-4-amine [1704];
5-(6-(difluoromethoxy)-1H-pyrrolo[2,3-b]pyridin-3-yl)-2-methyl-2H-indazole [1705];
2-(6-(difluoromethoxy)-1H-pyrrolo[2,3-b]pyridin-3-yl)thieno[2,3-c]pyridine [1706];
7-(6-(difluoromethoxy)-1H-pyrrolo[2,3-b]pyridin-3-yl)-3,4-dihydropyrrolo[1,2-a]pyrazin-1(2H)-one [1707];
6-(6-(difluoromethoxy)-1H-pyrrolo[2,3-b]pyridin-3-yl)imidazo[1,2-a]pyridine [1708];
N-(2,2-difluoroethyl)-6-(6-(difluoromethoxy)-1H-pyrrolo[2,3-b]pyridin-3-yl)imidazo[1,2-a]pyridine-3-carboxamide [1709];
6-(6-(difluoromethoxy)-1H-pyrrolo[2,3-b]pyridin-3-yl)imidazo[1,2-a]pyrimidine [1710];
4-fluoro-1-isopropyl-6-(6-(difluoromethoxy)-1H-pyrrolo[2,3-b]pyridin-3-yl)-2-methyl-1H-benzo[d]imidazole [1711];
4-fluoro-6-(6-(difluoromethoxy)-1H-pyrrolo[2,3-b]pyridin-3-yl)-2-methyl-1-(1-methylpiperidin-4-yl)-1H-benzo[d]imidazole [1712];
1-isopropyl-6-(6-(difluoromethoxy)-1H-pyrrolo[2,3-b]pyridin-3-yl)-2-methyl-1H-imidazo[4,5-b]pyridine [1713];
1-isopropyl-6-(6-(difluoromethoxy)-1H-pyrrolo[2,3-b]pyridin-3-yl)-2-methyl-1H-imidazo[4,5-c]pyridine [1714];
6-(6-(difluoromethoxy)-1H-pyrrolo[2,3-b]pyridin-3-yl)-[1,2,4]triazolo[1,5-a]pyridine [1715];
7-(6-(difluoromethoxy)-1H-pyrrolo[2,3-b]pyridin-3-yl)-[1,2,4]triazolo[1,5-a]pyridine [1716];
6-(6-(difluoromethoxy)-1H-pyrrolo[2,3-b]pyridin-3-yl)-1-methyl-1H-benzo[d][1,2,3]triazole [1717];
6-(6-(difluoromethoxy)-1H-pyrrolo[2,3-b]pyridin-3-yl)imidazo[1,2-b]pyridazine [1718];
6-(difluoromethoxy)-3-(pyrazolo[1,5-a]pyridin-5-yl)-1H-pyrrolo[2,3-b]pyridine [1719];
6-(difluoromethoxy)-3-(3-(1-methyl-1H-pyrazol-4-yl)pyrazolo[1,5-a]pyridin-5-yl)-1H-pyrrolo[2,3-b]pyridine [1720];
6-(difluoromethoxy)-3-(3-(pyridin-3-yl)pyrazolo[1,5-a]pyridin-5-yl)-1H-pyrrolo[2,3-b]pyridine [1721];
N-(2,2-difluoroethyl)-5-(6-(difluoromethoxy)-1H-pyrrolo[2,3-b]pyridin-3-yl)pyrazolo[1,5-a]pyridine-3-carboxamide [1722];
(R)-5-(6-(difluoromethoxy)-1H-pyrrolo[2,3-b]pyridin-3-yl)-N-(1,1,1-trifluoropropan-2-yl)pyrazolo[1,5-a]pyridine-3-carboxamide [1723];
5-(6-(difluoromethoxy)-1H-pyrrolo[2,3-b]pyridin-3-yl)-N-(1-methylpiperidin-4-yl)pyrazolo[1,5-a]pyridine-3-carboxamide [1724];
5-(6-(difluoromethoxy)-1H-pyrrolo[2,3-b]pyridin-3-yl)-N-(pyridin-3-yl)pyrazolo[1,5-a]pyridine-3-carboxamide [1725];
(5-(6-(difluoromethoxy)-1H-pyrrolo[2,3-b]pyridin-3-yl)pyrazolo[1,5-a]pyridin-3-yl)(piperidin-1-yl)methanone [1726];
(5-(6-(difluoromethoxy)-1H-pyrrolo[2,3-b]pyridin-3-yl)pyrazolo[1,5-a]pyridin-3-yl)(4-methylpiperazin-1-yl)methanone [1727];
(5-(6-(difluoromethoxy)-1H-pyrrolo[2,3-b]pyridin-3-yl)pyrazolo[1,5-a]pyridin-3-yl)(morpholino)methanone [1728];
6-(6-(difluoromethoxy)-1H-pyrrolo[2,3-b]pyridin-3-yl)spiro[indene-1,4′-piperidin]-3(2H)-one [1730];
6-(6-(difluoromethoxy)-1H-pyrrolo[2,3-b]pyridin-3-yl)-1′-methylspiro[indene-1,4′-piperidin]-3(2H)-one [1731];
5-(6-(difluoromethoxy)-1H-pyrrolo[2,3-b]pyridin-3-yl)isoindolin-1-one [1732];
5-(6-(difluoromethoxy)-1H-pyrrolo[2,3-b]pyridin-3-yl)-3,3-dimethylisoindolin-1-one [1733];
6′-(6-(difluoromethoxy)-1H-pyrrolo[2,3-b]pyridin-3-yl)spiro[cyclohexane-1,1′-isoindolin]-3′-one [1734];
3-(chroman-6-yl)-6-(difluoromethoxy)-1H-pyrrolo[2,3-b]pyridine [1735];
6-(6-(difluoromethoxy)-1H-pyrrolo[2,3-b]pyridin-3-yl)-3,4-dihydroisoquinolin-1(2H)-one [1736];
6-(6-(difluoromethoxy)-1H-pyrrolo[2,3-b]pyridin-3-yl)-4,4-dimethyl-3,4-dihydroisoquinolin-1(2H)-one [1737];
7-(6-(difluoromethoxy)-1H-pyrrolo[2,3-b]pyridin-3-yl)spiro[chromane-2,4′-piperidin]-4-one [1738];
8-(6-(difluoromethoxy)-1H-pyrrolo[2,3-b]pyridin-3-yl)-3,4-dihydrobenzo[f][1,4]oxazepin-5(2H)-one [1739];
6-(6-(difluoromethoxy)-1H-pyrrolo[2,3-b]pyridin-3-yl)quinoline [1740];
6-(6-(difluoromethoxy)-1H-pyrrolo[2,3-b]pyridin-3-yl)quinoxaline [1741];
6-(6-(difluoromethoxy)-1H-pyrrolo[2,3-b]pyridin-3-yl)quinazoline [1742];
4-methoxy-6-(6-(difluoromethoxy)-1H-pyrrolo[2,3-b]pyridin-3-yl)quinazoline [1743];
6-(6-(difluoromethoxy)-1H-pyrrolo[2,3-b]pyridin-3-yl)-4-(4-methylpiperazin-1-yl)quinazoline [1744];
6-(6-(difluoromethoxy)-1H-pyrrolo[2,3-b]pyridin-3-yl)-4-((1-methylpiperidin-4-yl)oxy)quinazoline [1745];
6-(6-(difluoromethoxy)-1H-pyrrolo[2,3-b]pyridin-3-yl)-N-(1-methylpiperidin-4-yl)quinazolin-4-amine [1746];
5-(6-ethoxy-1H-pyrrolo[2,3-b]pyridin-3-yl)-2-methyl-2H-indazole [1747];
2-(6-ethoxy-1H-pyrrolo[2,3-b]pyridin-3-yl)thieno[2,3-c]pyridine [1748];
7-(6-ethoxy-1H-pyrrolo[2,3-b]pyridin-3-yl)-3,4-dihydropyrrolo[1,2-a]pyrazin-1(2H)-one [1749];
6-(6-ethoxy-1H-pyrrolo[2,3-b]pyridin-3-yl)imidazo[1,2-a]pyridine [1750];
N-(2,2-difluoroethyl)-6-(6-ethoxy-1H-pyrrolo[2,3-b]pyridin-3-yl)imidazo[1,2-a]pyridine-3-carboxamide [1751];
6-(6-ethoxy-1H-pyrrolo[2,3-b]pyridin-3-yl)imidazo[1,2-a]pyrimidine [1752];
4-fluoro-1-isopropyl-6-(6-ethoxy-1H-pyrrolo[2,3-b]pyridin-3-yl)-2-methyl-1H-benzo[d]imidazole [1753];
4-fluoro-6-(6-ethoxy-1H-pyrrolo[2,3-b]pyridin-3-yl)-2-methyl-1-(1-methylpiperidin-4-yl)-1H-benzo[d]imidazole [1754];
1-isopropyl-6-(6-ethoxy-1H-pyrrolo[2,3-b]pyridin-3-yl)-2-methyl-1H-imidazo[4,5-b]pyridine [1755];
1-isopropyl-6-(6-ethoxy-1H-pyrrolo[2,3-b]pyridin-3-yl)-2-methyl-1H-imidazo[4,5-c]pyridine [1756];
6-(6-ethoxy-1H-pyrrolo[2,3-b]pyridin-3-yl)-[1,2,4]triazolo[1,5-a]pyridine [1757];
7-(6-ethoxy-1H-pyrrolo[2,3-b]pyridin-3-yl)-[1,2,4]triazolo[1,5-a]pyridine [1758];
6-(6-ethoxy-1H-pyrrolo[2,3-b]pyridin-3-yl)-1-methyl-1H-benzo[d][1,2,3]triazole [1759];
6-(6-ethoxy-1H-pyrrolo[2,3-b]pyridin-3-yl)imidazo[1,2-b]pyridazine [1760];
6-ethoxy-3-(pyrazolo[1,5-a]pyridin-5-yl)-1H-pyrrolo[2,3-b]pyridine [1761];
6-ethoxy-3-(3-(1-methyl-1H-pyrazol-4-yl)pyrazolo[1,5-a]pyridin-5-yl)-1H-pyrrolo[2,3-b]pyridine [1762];
6-ethoxy-3-(3-(pyridin-3-yl)pyrazolo[1,5-a]pyridin-5-yl)-1H-pyrrolo[2,3-b]pyridine [1763];
N-(2,2-difluoroethyl)-5-(6-ethoxy-1H-pyrrolo[2,3-b]pyridin-3-yl)pyrazolo[1,5-a]pyridine-3-carboxamide [1764];
(R)-5-(6-ethoxy-1H-pyrrolo[2,3-b]pyridin-3-yl)-N-(1,1,1-trifluoropropan-2-yl)pyrazolo[1,5-a]pyridine-3-carboxamide [1765];
5-(6-ethoxy-1H-pyrrolo[2,3-b]pyridin-3-yl)-N-(1-methylpiperidin-4-yl)pyrazolo[1,5-a]pyridine-3-carboxamide [1766];
5-(6-ethoxy-1H-pyrrolo[2,3-b]pyridin-3-yl)-N-(pyridin-3-yl)pyrazolo[1,5-a]pyridine-3-carboxamide [1767];
(5-(6-ethoxy-1H-pyrrolo[2,3-b]pyridin-3-yl)pyrazolo[1,5-a]pyridin-3-yl)(piperidin-1-yl)methanone [1768];
(5-(6-ethoxy-1H-pyrrolo[2,3-b]pyridin-3-yl)pyrazolo[1,5-a]pyridin-3-yl)(4-methylpiperazin-1-yl)methanone [1769];
(5-(6-ethoxy-1H-pyrrolo[2,3-b]pyridin-3-yl)pyrazolo[1,5-a]pyridin-3-yl)(morpholino)methanone [1770];
6-(6-ethoxy-1H-pyrrolo[2,3-b]pyridin-3-yl)spiro[indene-1,4′-piperidin]-3(2H)-one [1772];
6-(6-ethoxy-1H-pyrrolo[2,3-b]pyridin-3-yl)-1′-methylspiro[indene-1,4′-piperidin]-3(2H)-one [1773];
5-(6-ethoxy-1H-pyrrolo[2,3-b]pyridin-3-yl)isoindolin-1-one [1774];
5-(6-ethoxy-1H-pyrrolo[2,3-b]pyridin-3-yl)-3,3-dimethylisoindolin-1-one [1775];
6′-(6-ethoxy-1H-pyrrolo[2,3-b]pyridin-3-yl)spiro[cyclohexane-1,1′-isoindolin]-3′-one [1776];
3-(chroman-6-yl)-6-ethoxy-1H-pyrrolo[2,3-b]pyridine [1777];
6-(6-ethoxy-1H-pyrrolo[2,3-b]pyridin-3-yl)-3,4-dihydroisoquinolin-1(2H)-one [1778];
6-(6-ethoxy-1H-pyrrolo[2,3-b]pyridin-3-yl)-4,4-dimethyl-3,4-dihydroisoquinolin-1(2H)-one [1779];
7-(6-ethoxy-1H-pyrrolo[2,3-b]pyridin-3-yl)spiro[chromane-2,4′-piperidin]-4-one [1780];
8-(6-ethoxy-1H-pyrrolo[2,3-b]pyridin-3-yl)-3,4-dihydrobenzo[f][1,4]oxazepin-5(2H)-one [1781];
6-(6-ethoxy-1H-pyrrolo[2,3-b]pyridin-3-yl)quinoline [1782];
6-(6-ethoxy-1H-pyrrolo[2,3-b]pyridin-3-yl)quinoxaline [1783];
6-(6-ethoxy-1H-pyrrolo[2,3-b]pyridin-3-yl)quinazoline [1784];
4-methoxy-6-(6-ethoxy-1H-pyrrolo[2,3-b]pyridin-3-yl)quinazoline [1785];
6-(6-ethoxy-1H-pyrrolo[2,3-b]pyridin-3-yl)-4-(4-methylpiperazin-1-yl)quinazoline [1786];
6-(6-ethoxy-1H-pyrrolo[2,3-b]pyridin-3-yl)-4-((1-methylpiperidin-4-yl)oxy)quinazoline [1787];
6-(6-ethoxy-1H-pyrrolo[2,3-b]pyridin-3-yl)-N-(1-methylpiperidin-4-yl)quinazolin-4-amine [1788];
(3-(2-methyl-2H-indazol-5-yl)-1H-pyrrolo[2,3-b]pyridin-6-yl)methanol [1789];
(3-(thieno[2,3-c]pyridin-2-yl)-1H-pyrrolo[2,3-b]pyridin-6-yl)methanol [1790];
7-(6-(hydroxymethyl)-1H-pyrrolo[2,3-b]pyridin-3-yl)-3,4-dihydropyrrolo[1,2-a]pyrazin-1(2H)-one [1791];
(3-(imidazo[1,2-a]pyridin-6-yl)-1H-pyrrolo[2,3-b]pyridin-6-yl)methanol [1792];
N-(2,2-difluoroethyl)-6-(6-(hydroxymethyl)-1H-pyrrolo[2,3-b]pyridin-3-yl)imidazo[1,2-a]pyridine-3-carboxamide [1793];
(3-(imidazo[1,2-a]pyrimidin-6-yl)-1H-pyrrolo[2,3-b]pyridin-6-yl)methanol [1794];
(3-(4-fluoro-1-isopropyl-2-methyl-1H-benzo[d]imidazol-6-yl)-1H-pyrrolo[2,3-b]pyridin-6-yl)methanol [1795];
(3-(4-fluoro-2-methyl-1-(1-methylpiperidin-4-yl)-1H-benzo[d]imidazol-6-yl)-1H-pyrrolo[2,3-b]pyridin-6-yl)methanol [1796];
(3-(1-isopropyl-2-methyl-1H-imidazo[4,5-b]pyridin-6-yl)-1H-pyrrolo[2,3-b]pyridin-6-yl)methanol [1797];
(3-(1-isopropyl-2-methyl-1H-imidazo[4,5-c]pyridin-6-yl)-1H-pyrrolo[2,3-b]pyridin-6-yl)methanol [1798];
(3-([1,2,4]triazolo[1,5-a]pyridin-6-yl)-1H-pyrrolo[2,3-b]pyridin-6-yl)methanol [1799];
(3-([1,2,4]triazolo[1,5-a]pyridin-7-yl)-1H-pyrrolo[2,3-b]pyridin-6-yl)methanol [1800];
(3-(1-methyl-1H-benzo[d][1,2,3]triazol-6-yl)-1H-pyrrolo[2,3-b]pyridin-6-yl)methanol [1801];
(3-(imidazo[1,2-b]pyridazin-6-yl)-1H-pyrrolo[2,3-b]pyridin-6-yl)methanol [1802];
(3-(pyrazolo[1,5-a]pyridin-5-yl)-1H-pyrrolo[2,3-b]pyridin-6-yl)methanol [1803];
(3-(3-(1-methyl-1H-pyrazol-4-yl)pyrazolo[1,5-a]pyridin-5-yl)-1H-pyrrolo[2,3-b]pyridin-6-yl)methanol [1804];
(3-(3-(pyridin-3-yl)pyrazolo[1,5-a]pyridin-5-yl)-1H-pyrrolo[2,3-b]pyridin-6-yl)methanol [1805];
N-(2,2-difluoroethyl)-5-(6-(hydroxymethyl)-1H-pyrrolo[2,3-b]pyridin-3-yl)pyrazolo[1,5-a]pyridine-3-carboxamide [1806];
(R)-5-(6-(hydroxymethyl)-1H-pyrrolo[2,3-b]pyridin-3-yl)-N-(1,1,1-trifluoropropan-2-yl)pyrazolo[1,5-a]pyridine-3-carboxamide [1807];
5-(6-(hydroxymethyl)-1H-pyrrolo[2,3-b]pyridin-3-yl)-N-(1-methylpiperidin-4-yl)pyrazolo[1,5-a]pyridine-3-carboxamide [1808];
5-(6-(hydroxymethyl)-1H-pyrrolo[2,3-b]pyridin-3-yl)-N-(pyridin-3-yl)pyrazolo[1,5-a]pyridine-3-carboxamide [1809];
(5-(6-(hydroxymethyl)-1H-pyrrolo[2,3-b]pyridin-3-yl)pyrazolo[1,5-a]pyridin-3-yl)(piperidin-1-yl)methanone [1810];
(5-(6-(hydroxymethyl)-1H-pyrrolo[2,3-b]pyridin-3-yl)pyrazolo[1,5-a]pyridin-3-yl)(4-methylpiperazin-1-yl)methanone [1811];
(5-(6-(hydroxymethyl)-1H-pyrrolo[2,3-b]pyridin-3-yl)pyrazolo[1,5-a]pyridin-3-yl)(morpholino)methanone [1812];
6-(6-(hydroxymethyl)-1H-pyrrolo[2,3-b]pyridin-3-yl)spiro[indene-1,4′-piperidin]-3(2H)-one [1814];
6-(6-(hydroxymethyl)-1H-pyrrolo[2,3-b]pyridin-3-yl)-1′-methylspiro[indene-1,4′-piperidin]-3(2H)-one [1815];
5-(6-(hydroxymethyl)-1H-pyrrolo[2,3-b]pyridin-3-yl)isoindolin-1-one [1816];
5-(6-(hydroxymethyl)-1H-pyrrolo[2,3-b]pyridin-3-yl)-3,3-dimethylisoindolin-1-one [1817];
6′-(6-(hydroxymethyl)-1H-pyrrolo[2,3-b]pyridin-3-yl)spiro[cyclohexane-1,1′-isoindolin]-3′-one [1818];
(3-(chroman-6-yl)-1H-pyrrolo[2,3-b]pyridin-6-yl)methanol [1819];
6-(6-(hydroxymethyl)-1H-pyrrolo[2,3-b]pyridin-3-yl)-3,4-dihydroisoquinolin-1(2H)-one [1820];
6-(6-(hydroxymethyl)-1H-pyrrolo[2,3-b]pyridin-3-yl)-4,4-dimethyl-3,4-dihydroisoquinolin-1(2H)-one [1821];
7-(6-(hydroxymethyl)-1H-pyrrolo[2,3-b]pyridin-3-yl)spiro[chromane-2,4′-piperidin]-4-one [1822];
8-(6-(hydroxymethyl)-1H-pyrrolo[2,3-b]pyridin-3-yl)-3,4-dihydrobenzo[f][1,4]oxazepin-5(2H)-one [1823];
(3-(quinolin-6-yl)-1H-pyrrolo[2,3-b]pyridin-6-yl)methanol [1824];
(3-(quinoxalin-6-yl)-1H-pyrrolo[2,3-b]pyridin-6-yl)methanol [1825];
(3-(quinazolin-6-yl)-1H-pyrrolo[2,3-b]pyridin-6-yl)methanol [1826];
(3-(4-methoxyquinazolin-6-yl)-1H-pyrrolo[2,3-b]pyridin-6-yl)methanol [1827];
(3-(4-(4-methylpiperazin-1-yl)quinazolin-6-yl)-1H-pyrrolo[2,3-b]pyridin-6-yl)methanol [1828];
(3-(4-((1-methylpiperidin-4-yl)oxy)quinazolin-6-yl)-1H-pyrrolo[2,3-b]pyridin-6-yl)methanol [1829];
(3-(4-((1-methylpiperidin-4-yl)amino)quinazolin-6-yl)-1H-pyrrolo[2,3-b]pyridin-6-yl)methanol [1830];
1-methyl-N-(3-(2-methyl-2H-indazol-5-yl)-1H-pyrrolo[2,3-b]pyridin-6-yl)piperidine-4-carboxamide [1831];
1-methyl-N-(3-(thieno[2,3-c]pyridin-2-yl)-1H-pyrrolo[2,3-b]pyridin-6-yl)piperidine-4-carboxamide [1832];
1-methyl-N-(3-(1-oxo-1,2,3,4-tetrahydropyrrolo[1,2-a]pyrazin-7-yl)-1H-pyrrolo[2,3-b]pyridin-6-yl)piperidine-4-carboxamide [1833];
N-(3-(imidazo[1,2-a]pyridin-6-yl)-1H-pyrrolo[2,3-b]pyridin-6-yl)-1-methylpiperidine-4-carboxamide [1834];
N-(2,2-difluoroethyl)-6-(6-(1-methylpiperidine-4-carboxamido)-1H-pyrrolo[2,3-b]pyridin-3-yl)imidazo[1,2-a]pyridine-3-carboxamide [1835];
N-(3-(imidazo[1,2-a]pyrimidin-6-yl)-1H-pyrrolo[2,3-b]pyridin-6-yl)-1-methylpiperidine-4-carboxamide [1836];
N-(3-(4-fluoro-1-isopropyl-2-methyl-1H-benzo[d]imidazol-6-yl)-1H-pyrrolo[2,3-b]pyridin-6-yl)-1-methylpiperidine-4-carboxamide [1837];
N-(3-(4-fluoro-2-methyl-1-(1-methylpiperidin-4-yl)-1H-benzo[d]imidazol-6-yl)-1H-pyrrolo[2,3-b]pyridin-6-yl)-1-methylpiperidine-4-carboxamide [1838];
N-(3-(1-isopropyl-2-methyl-1H-imidazo[4,5-b]pyridin-6-yl)-1H-pyrrolo[2,3-b]pyridin-6-yl)-1-methylpiperidine-4-carboxamide [1839];
N-(3-(1-isopropyl-2-methyl-1H-imidazo[4,5-c]pyridin-6-yl)-1H-pyrrolo[2,3-b]pyridin-6-yl)-1-methylpiperidine-4-carboxamide [1840];
N-(3-([1,2,4]triazolo[1,5-a]pyridin-6-yl)-1H-pyrrolo[2,3-b]pyridin-6-yl)-1-methylpiperidine-4-carboxamide [1841];
N-(3-([1,2,4]triazolo[1,5-a]pyridin-7-yl)-1H-pyrrolo[2,3-b]pyridin-6-yl)-1-methylpiperidine-4-carboxamide [1842];
1-methyl-N-(3-(1-methyl-1H-benzo[d][1,2,3]triazol-6-yl)-1H-pyrrolo[2,3-b]pyridin-6-yl)piperidine-4-carboxamide [1843];
N-(3-(imidazo[1,2-b]pyridazin-6-yl)-1H-pyrrolo[2,3-b]pyridin-6-yl)-1-methylpiperidine-4-carboxamide [1844];
1-methyl-N-(3-(pyrazolo[1,5-a]pyridin-5-yl)-1H-pyrrolo[2,3-b]pyridin-6-yl)piperidine-4-carboxamide [1845];
1-methyl-N-(3-(3-(1-methyl-1H-pyrazol-4-yl)pyrazolo[1,5-a]pyridin-5-yl)-1H-pyrrolo[2,3-b]pyridin-6-yl)piperidine-4-carboxamide [1846];
1-methyl-N-(3-(3-(pyridin-3-yl)pyrazolo[1,5-a]pyridin-5-yl)-1H-pyrrolo[2,3-b]pyridin-6-yl)piperidine-4-carboxamide [1847];
N-(2,2-difluoroethyl)-5-(6-(1-methylpiperidine-4-carboxamido)-1H-pyrrolo[2,3-b]pyridin-3-yl)pyrazolo[1,5-a]pyridine-3-carboxamide [1848];
(R)-5-(6-(1-methylpiperidine-4-carboxamido)-1H-pyrrolo[2,3-b]pyridin-3-yl)-N-(1,1,1-trifluoropropan-2-yl)pyrazolo[1,5-a]pyridine-3-carboxamide [1849];
N-(1-methylpiperidin-4-yl)-5-(6-(1-methylpiperidine-4-carboxamido)-1H-pyrrolo[2,3-b]pyridin-3-yl)pyrazolo[1,5-a]pyridine-3-carboxamide [1850];
5-(6-(1-methylpiperidine-4-carboxamido)-1H-pyrrolo[2,3-b]pyridin-3-yl)-N-(pyridin-3-yl)pyrazolo[1,5-a]pyridine-3-carboxamide [1851];
1-methyl-N-(3-(3-(piperidine-1-carbonyl)pyrazolo[1,5-a]pyridin-5-yl)-1H-pyrrolo[2,3-b]pyridin-6-yl)piperidine-4-carboxamide [1852];
1-methyl-N-(3-(3-(4-methylpiperazine-1-carbonyl)pyrazolo[1,5-a]pyridin-5-yl)-1H-pyrrolo[2,3-b]pyridin-6-yl)piperidine-4-carboxamide [1853];
1-methyl-N-(3-(3-(morpholine-4-carbonyl)pyrazolo[1,5-a]pyridin-5-yl)-1H-pyrrolo[2,3-b]pyridin-6-yl)piperidine-4-carboxamide [1854];
1-methyl-N-(3-(3-oxo-2,3-dihydrospiro[indene-1,4′-piperidin]-6-yl)-1H-pyrrolo[2,3-b]pyridin-6-yl)piperidine-4-carboxamide [1856];
1-methyl-N-(3-(1′-methyl-3-oxo-2,3-dihydrospiro[indene-1,4′-piperidin]-6-yl)-1H-pyrrolo[2,3-b]pyridin-6-yl)piperidine-4-carboxamide [1857];
1-methyl-N-(3-(1-oxoisoindolin-5-yl)-1H-pyrrolo[2,3-b]pyridin-6-yl)piperidine-4-carboxamide [1858];
N-(3-(3,3-dimethyl-1-oxoisoindolin-5-yl)-1H-pyrrolo[2,3-b]pyridin-6-yl)-1-methylpiperidine-4-carboxamide [1859];
1-methyl-N-(3-(3′-oxospiro[cyclohexane-1,1′-isoindolin]-6′-yl)-1H-pyrrolo[2,3-b]pyridin-6-yl)piperidine-4-carboxamide [1860];
N-(3-(chroman-6-yl)-1H-pyrrolo[2,3-b]pyridin-6-yl)-1-methylpiperidine-4-carboxamide [1861];
1-methyl-N-(3-(1-oxo-1,2,3,4-tetrahydroisoquinolin-6-yl)-1H-pyrrolo[2,3-b]pyridin-6-yl)piperidine-4-carboxamide [1862];
N-(3-(4,4-dimethyl-1-oxo-1,2,3,4-tetrahydroisoquinolin-6-yl)-1H-pyrrolo[2,3-b]pyridin-6-yl)-1-methylpiperidine-4-carboxamide [1863];
1-methyl-N-(3-(4-oxospiro[chromane-2,4′-piperidin]-7-yl)-1H-pyrrolo[2,3-b]pyridin-6-yl)piperidine-4-carboxamide [1864];
1-methyl-N-(3-(5-oxo-2,3,4,5-tetrahydrobenzo[f][1,4]oxazepin-8-yl)-1H-pyrrolo[2,3-b]pyridin-6-yl)piperidine-4-carboxamide [1865];
1-methyl-N-(3-(quinolin-6-yl)-1H-pyrrolo[2,3-b]pyridin-6-yl)piperidine-4-carboxamide [1866];
1-methyl-N-(3-(quinoxalin-6-yl)-1H-pyrrolo[2,3-b]pyridin-6-yl)piperidine-4-carboxamide [1867];
1-methyl-N-(3-(quinazolin-6-yl)-1H-pyrrolo[2,3-b]pyridin-6-yl)piperidine-4-carboxamide [1868];
N-(3-(4-methoxyquinazolin-6-yl)-1H-pyrrolo[2,3-b]pyridin-6-yl)-1-methylpiperidine-4-carboxamide [1869];
1-methyl-N-(3-(4-(4-methylpiperazin-1-yl)quinazolin-6-yl)-1H-pyrrolo[2,3-b]pyridin-6-yl)piperidine-4-carboxamide [1870];
1-methyl-N-(3-(4-((1-methylpiperidin-4-yl)oxy)quinazolin-6-yl)-1H-pyrrolo[2,3-b]pyridin-6-yl)piperidine-4-carboxamide [1871];
1-methyl-N-(3-(4-((1-methylpiperidin-4-yl)amino)quinazolin-6-yl)-1H-pyrrolo[2,3-b]pyridin-6-yl)piperidine-4-carboxamide [1872];
N-(3-(2-methyl-2H-indazol-5-yl)-1H-pyrrolo[2,3-b]pyridin-6-yl)isonicotinamide [1873];
N-(3-(thieno[2,3-c]pyridin-2-yl)-1H-pyrrolo[2,3-b]pyridin-6-yl)isonicotinamide [1874];
N-(3-(1-oxo-1,2,3,4-tetrahydropyrrolo[1,2-a]pyrazin-7-yl)-1H-pyrrolo[2,3-b]pyridin-6-yl)isonicotinamide [1875];
N-(3-(imidazo[1,2-a]pyridin-6-yl)-1H-pyrrolo[2,3-b]pyridin-6-yl)isonicotinamide [1876];
N-(2,2-difluoroethyl)-6-(6-(isonicotinamido)-1H-pyrrolo[2,3-b]pyridin-3-yl)imidazo[1,2-a]pyridine-3-carboxamide [1877];
N-(3-(imidazo[1,2-a]pyrimidin-6-yl)-1H-pyrrolo[2,3-b]pyridin-6-yl)isonicotinamide [1878];
N-(3-(4-fluoro-1-isopropyl-2-methyl-1H-benzo[d]imidazol-6-yl)-1H-pyrrolo[2,3-b]pyridin-6-yl)isonicotinamide [1879];
N-(3-(4-fluoro-2-methyl-1-(1-methylpiperidin-4-yl)-1H-benzo[d]imidazol-6-yl)-1H-pyrrolo[2,3-b]pyridin-6-yl)isonicotinamide [1880];
N-(3-(1-isopropyl-2-methyl-1H-imidazo[4,5-b]pyridin-6-yl)-1H-pyrrolo[2,3-b]pyridin-6-yl)isonicotinamide [1881];
N-(3-(1-isopropyl-2-methyl-1H-imidazo[4,5-c]pyridin-6-yl)-1H-pyrrolo[2,3-b]pyridin-6-yl)isonicotinamide [1882];
N-(3-([1,2,4]triazolo[1,5-a]pyridin-6-yl)-1H-pyrrolo[2,3-b]pyridin-6-yl)isonicotinamide [1883];
N-(3-([1,2,4]triazolo[1,5-a]pyridin-7-yl)-1H-pyrrolo[2,3-b]pyridin-6-yl)isonicotinamide [1884];
N-(3-(1-methyl-1H-benzo[d][1,2,3]triazol -6-yl)-1H-pyrrolo[2,3-b]pyridin-6-yl)isonicotinamide [1885];
N-(3-(imidazo[1,2-b]pyridazin-6-yl)-1H-pyrrolo[2,3-b]pyridin-6-yl)isonicotinamide [1886];
N-(3-(pyrazolo[1,5-a]pyridin-5-yl)-1H-pyrrolo[2,3-b]pyridin-6-yl)isonicotinamide [1887];
N-(3-(3-(1-methyl-1H-pyrazol-4-yl)pyrazolo[1,5-a]pyridin-5-yl)-1H-pyrrolo[2,3-b]pyridin-6-yl)isonicotinamide [1888];
N-(3-(3-(pyridin-3-yl)pyrazolo[1,5-a]pyridin-5-yl)-1H-pyrrolo[2,3-b]pyridin-6-yl)isonicotinamide [1889];
N-(2,2-difluoroethyl)-5-(6-(isonicotinamido)-1H-pyrrolo[2,3-b]pyridin-3-yl)pyrazolo[1,5-a]pyridine-3-carboxamide [1890];
(R)-5-(6-(isonicotinamido)-1H-pyrrolo[2,3-b]pyridin-3-yl)-N-(1,1,1-trifluoropropan-2-yl)pyrazolo[1,5-a]pyridine-3-carboxamide [1891];
5-(6-(isonicotinamido)-1H-pyrrolo[2,3-b]pyridin-3-yl)-N-(1-methylpiperidin-4-yl)pyrazolo[1,5-a]pyridine-3-carboxamide [1892];
5-(6-(isonicotinamido)-1H-pyrrolo[2,3-b]pyridin-3-yl)-N-(pyridin-3-yl)pyrazolo[1,5-a]pyridine-3-carboxamide [1893];
N-(3-(3-(piperidine-1-carbonyl)pyrazolo[1,5-a]pyridin-5-yl)-1H-pyrrolo[2,3-b]pyridin-6-yl)isonicotinamide [1894];
N-(3-(3-(4-methylpiperazine-1-carbonyl)pyrazolo[1,5-a]pyridin-5-yl)-1H-pyrrolo[2,3-b]pyridin-6-yl)isonicotinamide [1895];
N-(3-(3-(morpholine-4-carbonyl)pyrazolo[1,5-a]pyridin-5-yl)-1H-pyrrolo[2,3-b]pyridin-6-yl)isonicotinamide [1896];
N-(3-(3-oxo-2,3-dihydrospiro[indene-1,4′-piperidin]-6-yl)-1H-pyrrolo[2,3-b]pyridin-6-yl)isonicotinamide [1898];
N-(3-(1′-methyl-3-oxo-2,3-dihydrospiro[indene-1,4′-piperidin]-6-yl)-1H-pyrrolo[2,3-b]pyridin-6-yl)isonicotinamide [1899];
N-(3-(1-oxoisoindolin-5-yl)-1H-pyrrolo[2,3-b]pyridin-6-yl)isonicotinamide [1900];
N-(3-(3,3-dimethyl-1-oxoisoindolin-5-yl)-1H-pyrrolo[2,3-b]pyridin-6-yl)isonicotinamide [1901];
N-(3-(3′-oxospiro[cyclohexane-1,1′-isoindolin]-6′-yl)-1H-pyrrolo[2,3-b]pyridin-6-yl)isonicotinamide [1902];
N-(3-(chroman-6-yl)-1H-pyrrolo[2,3-b]pyridin-6-yl)isonicotinamide [1903];
N-(3-(1-oxo-1,2,3,4-tetrahydroisoquinolin-6-yl)-1H-pyrrolo[2,3-b]pyridin-6-yl)isonicotinamide [1904];
N-(3-(4,4-dimethyl-1-oxo-1,2,3,4-tetrahydroisoquinolin-6-yl)-1H-pyrrolo[2,3-b]pyridin-6-yl)isonicotinamide [1905];
N-(3-(4-oxospiro[chromane-2,4′-piperidin]-7-yl)-1H-pyrrolo[2,3-b]pyridin-6-yl)isonicotinamide [1906];
N-(3-(5-oxo-2,3,4,5-tetrahydrobenzo[f][1,4]oxazepin-8-yl)-1H-pyrrolo[2,3-b]pyridin-6-yl)isonicotinamide [1907];
N-(3-(quinolin-6-yl)-1H-pyrrolo[2,3-b]pyridin-6-yl)isonicotinamide [1908];
N-(3-(quinoxalin-6-yl)-1H-pyrrolo[2,3-b]pyridin-6-yl)isonicotinamide [1909];
N-(3-(quinazolin-6-yl)-1H-pyrrolo[2,3-b]pyridin-6-yl)isonicotinamide [1910];
N-(3-(4-methoxyquinazolin-6-yl)-1H-pyrrolo[2,3-b]pyridin-6-yl)isonicotinamide [1911];
N-(3-(4-(4-methylpiperazin-1-yl)quinazolin-6-yl)-1H-pyrrolo[2,3-b]pyridin-6-yl)isonicotinamide [1912];
N-(3-(4-((1-methylpiperidin-4-yl)oxy)quinazolin-6-yl)-1H-pyrrolo[2,3-b]pyridin-6-yl)isonicotinamide [1913];
N-(3-(4-((1-methylpiperidin-4-yl)amino)quinazolin-6-yl)-1H-pyrrolo[2,3-b]pyridin-6-yl)isonicotinamide [1914];
3-(2-methyl-2H-indazol-5-yl)-N-(3-(4-methylpiperazin-1-yl)phenyl)-1H-pyrrolo[2,3-b]pyridin-6-amine [1915];
N-(3-(4-methylpiperazin-1-yl)phenyl)-3-(thieno[2,3-c]pyridin-2-yl)-1H-pyrrolo[2,3-b]pyridin-6-amine [1916];
7-(6-((3-(4-methylpiperazin-1-yl)phenyl)amino)-1H-pyrrolo[2,3-b]pyridin-3-yl)-3,4-dihydropyrrolo[1,2-a]pyrazin-1(2H)-one [1917];
3-(imidazo[1,2-a]pyridin-6-yl)-N-(3-(4-methylpiperazin-1-yl)phenyl)-1H-pyrrolo[2,3-b]pyridin-6-amine [1918];
N-(2,2-difluoroethyl)-6-(6-((3-(4-methylpiperazin-1-yl)phenyl)amino)-1H-pyrrolo[2,3-b]pyridin-3-yl)imidazo[1,2-a]pyridine-3-carboxamide [1919];
3-(imidazo[1,2-a]pyrimidin-6-yl)-N-(3-(4-methylpiperazin-1-yl)phenyl)-1H-pyrrolo[2,3-b]pyridin-6-amine [1920];
3-(4-fluoro-1-isopropyl-2-methyl-1H-benzo[d]imidazol-6-yl)-N-(3-(4-methylpiperazin-1-yl)phenyl)-1H-pyrrolo[2,3-b]pyridin-6-amine [1921];
3-(4-fluoro-2-methyl-1-(1-methylpiperidin-4-yl)-1H-benzo[d]imidazol-6-yl)-N-(3-(4-methylpiperazin-1-yl)phenyl)-1H-pyrrolo[2,3-b]pyridin-6-amine [1922];
3-(1-isopropyl-2-methyl-1H-imidazo[4,5-b]pyridin-6-yl)-N-(3-(4-methylpiperazin-1-yl)phenyl)-1H-pyrrolo[2,3-b]pyridin-6-amine [1923];
3-(1-isopropyl-2-methyl-1H-imidazo[4,5-c]pyridin-6-yl)-N-(3-(4-methylpiperazin-1-yl)phenyl)-1H-pyrrolo[2,3-b]pyridin-6-amine [1924];
3-([1,2,4]triazolo[1,5-a]pyridin-6-yl)-N-(3-(4-methylpiperazin-1-yl)phenyl)-1H-pyrrolo[2,3-b]pyridin-6-amine [1925];
3-([1,2,4]triazolo[1,5-a]pyridin-7-yl)-N-(3-(4-methylpiperazin-1-yl)phenyl)-1H-pyrrolo[2,3-b]pyridin-6-amine [1926];
3-(1-methyl-1H-benzo[d][1,2,3]triazol-6-yl)-N-(3-(4-methylpiperazin-1-yl)phenyl)-1H-pyrrolo[2,3-b]pyridin-6-amine [1927];
3-(imidazo[1,2-b]pyridazin-6-yl)-N-(3-(4-methylpiperazin-1-yl)phenyl)-1H-pyrrolo[2,3-b]pyridin-6-amine [1928];
N-(3-(4-methylpiperazin-1-yl)phenyl)-3-(pyrazolo[1,5-a]pyridin-5-yl)-1H-pyrrolo[2,3-b]pyridin-6-amine [1929];
3-(3-(1-methyl-1H-pyrazol-4-yl)pyrazolo[1,5-a]pyridin-5-yl)-N-(3-(4-methylpiperazin-1-yl)phenyl)-1H-pyrrolo[2,3-b]pyridin-6-amine [1930];
N-(3-(4-methylpiperazin-1-yl)phenyl)-3-(3-(pyridin-3-yl)pyrazolo[1,5-a]pyridin-5-yl)-1H-pyrrolo[2,3-b]pyridin-6-amine [1931];
N-(2,2-difluoroethyl)-5-(6-((3-(4-methylpiperazin-1-yl)phenyl)amino)-1H-pyrrolo[2,3-b]pyridin-3-yl)pyrazolo[1,5-a]pyridine-3-carboxamide [1932];
(R)-5-(6-((3-(4-methylpiperazin-1-yl)phenyl)amino)-1H-pyrrolo[2,3-b]pyridin-3-yl)-N-(1,1,1-trifluoropropan-2-yl)pyrazolo[1,5-a]pyridine-3-carboxamide [1933];
5-(6-((3-(4-methylpiperazin-1-yl)phenyl)amino)-1H-pyrrolo[2,3-b]pyridin-3-yl)-N-(1-methylpiperidin-4-yl)pyrazolo[1,5-a]pyridine-3-carboxamide [1934];
5-(6-((3-(4-methylpiperazin-1-yl)phenyl)amino)-1H-pyrrolo[2,3-b]pyridin-3-yl)-N-(pyridin-3-yl)pyrazolo[1,5-a]pyridine-3-carboxamide [1935];
(5-(6-((3-(4-methylpiperazin-1-yl)phenyl)amino)-1H-pyrrolo[2,3-b]pyridin-3-yl)pyrazolo[1,5-a]pyridin-3-yl)(piperidin-1-yl)methanone [1936];
(4-methylpiperazin-1-yl)(5-(6-((3-(4-methylpiperazin-1-yl)phenyl)amino)-1H-pyrrolo[2,3-b]pyridin-3-yl)pyrazolo[1,5-a]pyridin-3-yl)methanone [1937];
(5-(6-((3-(4-methylpiperazin-1-yl)phenyl)amino)-1H-pyrrolo[2,3-b]pyridin-3-yl)pyrazolo[1,5-a]pyridin-3-yl)(morpholino)methanone [1938];
6-(6-((3-(4-methylpiperazin-1-yl)phenyl)amino)-1H-pyrrolo[2,3-b]pyridin-3-yl)spiro[indene-1,4′-piperidin]-3(2H)-one [1940];
1′-methyl-6-(6-((3-(4-methylpiperazin-1-yl)phenyl)amino)-1H-pyrrolo[2,3-b]pyridin-3-yl)spiro[indene-1,4′-piperidin]-3(2H)-one [1941];
5-(6-((3-(4-methylpiperazin-1-yl)phenyl)amino)-1H-pyrrolo[2,3-b]pyridin-3-yl)isoindolin-1-one [1942];
3,3-dimethyl-5-(6-((3-(4-methylpiperazin-1-yl)phenyl)amino)-1H-pyrrolo[2,3-b]pyridin-3-yl)isoindolin-1-one [1943];
6′-(6-((3-(4-methylpiperazin-1-yl)phenyl)amino)-1H-pyrrolo[2,3-b]pyridin-3-yl)spiro[cyclohexane-1,1′-isoindolin]-3′-one [1944];
3-(chroman-6-yl)-N-(3-(4-methylpiperazin-1-yl)phenyl)-1H-pyrrolo[2,3-b]pyridin-6-amine [1945];
6-(6-((3-(4-methylpiperazin-1-yl)phenyl)amino)-1H-pyrrolo[2,3-b]pyridin-3-yl)-3,4-dihydroisoquinolin-1(2H)-one [1946];
4,4-dimethyl-6-(6-((3-(4-methylpiperazin-1-yl)phenyl)amino)-1H-pyrrolo[2,3-b]pyridin-3-yl)-3,4-dihydroisoquinolin-1(2H)-one [1947];
7-(6-((3-(4-methylpiperazin-1-yl)phenyl)amino)-1H-pyrrolo[2,3-b]pyridin-3-yl)spiro[chromane-2,4′-piperidin]-4-one [1948];
8-(6-((3-(4-methylpiperazin-1-yl)phenyl)amino)-1H-pyrrolo[2,3-b]pyridin-3-yl)-3,4-dihydrobenzo[1,4]oxazepin-5(2H)-one [1949];
N-(3-(4-methylpiperazin-1-yl)phenyl)-3-(quinolin-6-yl)-1H-pyrrolo[2,3-b]pyridin-6-amine [1950];
N-(3-(4-methylpiperazin-1-yl)phenyl)-3-(quinoxalin-6-yl)-1H-pyrrolo[2,3-b]pyridin-6-amine [1951];
N-(3-(4-methylpiperazin-1-yl)phenyl)-3-(quinazolin-6-yl)-1H-pyrrolo[2,3-b]pyridin-6-amine [1952];
3-(4-methoxyquinazolin-6-yl)-N-(3-(4-methylpiperazin-1-yl)phenyl)-1H-pyrrolo[2,3-b]pyridin-6-amine [1953];
N-(3-(4-methylpiperazin-1-yl)phenyl)-3-(4-(4-methylpiperazin-1-yl)quinazolin-6-yl)-1H-pyrrolo[2,3-b]pyridin-6-amine [1954];
N-(3-(4-methylpiperazin-1-yl)phenyl)-3-(4-((1-methylpiperidin-4-yl)oxy)quinazolin-6-yl)-1H-pyrrolo[2,3-b]pyridin-6-amine [1955];
6-(6-((3-(4-methylpiperazin-1-yl)phenyl)amino)-1H-pyrrolo[2,3-b]pyridin-3-yl)-N-(1-methylpiperidin-4-yl)quinazolin-4-amine [1956];
2-methyl-5-(6-(1-methyl-1H-pyrazol-4-yl)-1H-pyrrolo[2,3-b]pyridin-3-yl)-2H-indazole [1957];
2-(6-(1-methyl-1H-pyrazol-4-yl)-1H-pyrrolo[2,3-b]pyridin-3-yl)thieno[2,3-c]pyridine [1958];
7-(6-(1-methyl-1H-pyrazol-4-yl)-1H-pyrrolo[2,3-b]pyridin-3-yl)-3,4-dihydropyrrolo[1,2-a]pyrazin-1(2H)-one [1959];
6-(6-(1-methyl-1H-pyrazol-4-yl)-1H-pyrrolo[2,3-b]pyridin-3-yl)imidazo[1,2-a]pyridine [1960];
N-(2,2-difluoroethyl)-6-(6-(1-methyl-1H-pyrazol-4-yl)-1H-pyrrolo[2,3-b]pyridin-3-yl)imidazo[1,2-a]pyridine-3-carboxamide [1961];
6-(6-(1-methyl-1H-pyrazol-4-yl)-1H-pyrrolo[2,3-b]pyridin-3-yl)imidazo[1,2-a]pyrimidine [1962];
4-fluoro-1-isopropyl-2-methyl-6-(6-(1-methyl-1H-pyrazol-4-yl)-1H-pyrrolo[2,3-b]pyridin-3-yl)-1H-benzo[d]imidazole [1963];
4-fluoro-2-methyl-6-(6-(1-methyl-1H-pyrazol-4-yl)-1H-pyrrolo[2,3-b]pyridin-3-yl)-1-(1-methylpiperidin-4-yl)-1H-benzo[d]imidazole [1964];
1-isopropyl-2-methyl-6-(6-(1-methyl-1H-pyrazol-4-yl)-1H-pyrrolo[2,3-b]pyridin-3-yl)-1H-imidazo[4,5-b]pyridine [1965];
1-isopropyl-2-methyl-6-(6-(1-methyl-1H-pyrazol-4-yl)-1H-pyrrolo[2,3-b]pyridin-3-yl)-1H-imidazo[4,5-c]pyridine [1966];
6-(6-(1-methyl-1H-pyrazol-4-yl)-1H-pyrrolo[2,3-b]pyridin-3-yl)-[1,2,4]triazolo[1,5-a]pyridine [1967];
7-(6-(1-methyl-1H-pyrazol-4-yl)-1H-pyrrolo[2,3-b]pyridin-3-yl)-[1,2,4]triazolo[1,5-a]pyridine [1968];
1-methyl-6-(6-(1-methyl-1H-pyrazol-4-yl)-1H-pyrrolo[2,3-b]pyridin-3-yl)-1H-benzo[d][1,2,3]triazole [1969];
6-(6-(1-methyl-1H-pyrazol-4-yl)-1H-pyrrolo[2,3-b]pyridin-3-yl)imidazo[1,2-b]pyridazine [1970];
6-(1-methyl-1H-pyrazol-4-yl)-3-(pyrazolo[1,5-a]pyridin-5-yl)-1H-pyrrolo[2,3-b]pyridine [1971];
6-(1-methyl-1H-pyrazol-4-yl)-3-(3-(1-methyl-1H-pyrazol-4-yl)pyrazolo[1,5-a]pyridin-5-yl)-1H-pyrrolo[2,3-b]pyridine [1972];
6-(1-methyl-1H-pyrazol-4-yl)-3-(3-(pyridin-3-yl)pyrazolo[1,5-a]pyridin-5-yl)-1H-pyrrolo[2,3-b]pyridine [1973];
N-(2,2-difluoroethyl)-5-(6-(1-methyl-1H-pyrazol-4-yl)-1H-pyrrolo[2,3-b]pyridin-3-yl)pyrazolo[1,5-a]pyridine-3-carboxamide [1974];
(R)-5-(6-(1-methyl-1H-pyrazol-4-yl)-1H-pyrrolo[2,3-b]pyridin-3-yl)-N-(1,1,1-trifluoropropan-2-yl)pyrazolo[1,5-a]pyridine-3-carboxamide [1975];
5-(6-(1-methyl-1H-pyrazol-4-yl)-1H-pyrrolo[2,3-b]pyridin-3-yl)-N-(1-methylpiperidin-4-yl)pyrazolo[1,5-a]pyridine-3-carboxamide [1976];
5-(6-(1-methyl-1H-pyrazol-4-yl)-1H-pyrrolo[2,3-b]pyridin-3-yl)-N-(pyridin-3-yl)pyrazolo[1,5-a]pyridine-3-carboxamide [1977];
(5-(6-(1-methyl-1H-pyrazol-4-yl)-1H-pyrrolo[2,3-b]pyridin-3-yl)pyrazolo[1,5-a]pyridin-3-yl)(piperidin-1-yl)methanone [1978];
(5-(6-(1-methyl-1H-pyrazol-4-yl)-1H-pyrrolo[2,3-b]pyridin-3-yl)pyrazolo[1,5-a]pyridin-3-yl)(4-methylpiperazin-1-yl)methanone [1979];
(5-(6-(1-methyl-1H-pyrazol-4-yl)-1H-pyrrolo[2,3-b]pyridin-3-yl)pyrazolo[1,5-a]pyridin-3-yl)(morpholino)methanone [1980];
6-(6-(1-methyl-1H-pyrazol-4-yl)-1H-pyrrolo[2,3-b]pyridin-3-yl)spiro[indene-1,4′-piperidin]-3(2H)-one [1982];
1′-methyl-6-(6-(1-methyl-1H-pyrazol-4-yl)-1H-pyrrolo[2,3-b]pyridin-3-yl)spiro[indene-1,4′-piperidin]-3(2H)-one [1983];
5-(6-(1-methyl-1H-pyrazol-4-yl)-1H-pyrrolo[2,3-b]pyridin-3-yl)isoindolin-1-one [1984];
3,3-dimethyl-5-(6-(1-methyl-1H-pyrazol-4-yl)-1H-pyrrolo[2,3-b]pyridin-3-yl)isoindolin-1-one [1985];
6′-(6-(1-methyl-1H-pyrazol-4-yl)-1H-pyrrolo[2,3-b]pyridin-3-yl)spiro[cyclohexane-1,1′-isoindolin]-3′-one [1986];
3-(chroman-6-yl)-6-(1-methyl-1H-pyrazol-4-yl)-1H-pyrrolo[2,3-b]pyridine [1987];
6-(6-(1-methyl-1H-pyrazol-4-yl)-1H-pyrrolo[2,3-b]pyridin-3-yl)-3,4-dihydroisoquinolin-1(2H)-one [1988];
4,4-dimethyl-6-(6-(1-methyl-1H-pyrazol-4-yl)-1H-pyrrolo[2,3-b]pyridin-3-yl)-3,4-dihydroisoquinolin-1(2H)-one [1989];
7-(6-(1-methyl-1H-pyrazol-4-yl)-1H-pyrrolo[2,3-b]pyridin-3-yl)spiro[chromane-2,4′-piperidin]-4-one [1990];
8-(6-(1-methyl-1H-pyrazol-4-yl)-1H-pyrrolo[2,3-b]pyridin-3-yl)-3,4-dihydrobenzo[1,4]oxazepin-5(2H)-one [1991];
6-(6-(1-methyl-1H-pyrazol-4-yl)-1H-pyrrolo[2,3-b]pyridin-3-yl)quinoline [1992];
6-(6-(1-methyl-1H-pyrazol-4-yl)-1H-pyrrolo[2,3-b]pyridin-3-yl)quinoxaline [1993];
6-(6-(1-methyl-1H-pyrazol-4-yl)-1H-pyrrolo[2,3-b]pyridin-3-yl)quinazoline [1994];
4-methoxy-6-(6-(1-methyl-1H-pyrazol-4-yl)-1H-pyrrolo[2,3-b]pyridin-3-yl)quinazoline [1995];
6-(6-(1-methyl-1H-pyrazol-4-yl)-1H-pyrrolo[2,3-b]pyridin-3-yl)-4-(4-methylpiperazin-1-yl)quinazoline [1996];
6-(6-(1-methyl-1H-pyrazol-4-yl)-1H-pyrrolo[2,3-b]pyridin-3-yl)-4-((1-methylpiperidin-4-yl)oxy)quinazoline [1997];
6-(6-(1-methyl-1H-pyrazol-4-yl)-1H-pyrrolo[2,3-b]pyridin-3-yl)-N-(1-methylpiperidin-4-yl)quinazolin-4-amine [1998];
2-methyl-5-(6-phenyl-1H-pyrrolo[2,3-b]pyridin-3-yl)-2H-indazole [1999];
2-(6-phenyl-1H-pyrrolo[2,3-b]pyridin-3-yl)thieno[2,3-c]pyridine [2000];
7-(6-phenyl-1H-pyrrolo[2,3-b]pyridin-3-yl)-3,4-dihydropyrrolo[1,2-a]pyrazin-1(2H)-one [2001];
6-(6-phenyl-1H-pyrrolo[2,3-b]pyridin-3-yl)imidazo[1,2-a]pyridine [2002];
N-(2,2-difluoroethyl)-6-(6-phenyl-1H-pyrrolo[2,3-b]pyridin-3-yl)imidazo[1,2-a]pyridine-3-carboxamide [2003];
6-(6-phenyl-1H-pyrrolo[2,3-b]pyridin-3-yl)imidazo[1,2-a]pyrimidine [2004];
4-fluoro-1-isopropyl-2-methyl-6-(6-phenyl-1H-pyrrolo[2,3-b]pyridin-3-yl)-1H-benzo[d]imidazole [2005];
4-fluoro-2-methyl-1-(1-methylpiperidin-4-yl)-6-(6-phenyl-1H-pyrrolo[2,3-b]pyridin-3-yl)-1H-benzo[d]imidazole [2006];
1-isopropyl-2-methyl-6-(6-phenyl-1H-pyrrolo[2,3-b]pyridin-3-yl)-1H-imidazo[4,5-b]pyridine [2007];
1-isopropyl-2-methyl-6-(6-phenyl-1H-pyrrolo[2,3-b]pyridin-3-yl)-1H-imidazo[4,5-c]pyridine [2008];
6-(6-phenyl-1H-pyrrolo[2,3-b]pyridin-3-yl)-[1,2,4]triazolo[1,5-a]pyridine [2009];
7-(6-phenyl-1H-pyrrolo[2,3-b]pyridin-3-yl)-[1,2,4]triazolo[1,5-a]pyridine [2010];
1-methyl-6-(6-phenyl-1H-pyrrolo[2,3-b]pyridin-3-yl)-1H-benzo[d][1,2,3]triazole [2011];
6-(6-phenyl-1H-pyrrolo[2,3-b]pyridin-3-yl)imidazo[1,2-b]pyridazine [2012];
6-phenyl-3-(pyrazolo[1,5-a]pyridin-5-yl)-1H-pyrrolo[2,3-b]pyridine [2013];
3-(3-(1-methyl-1H-pyrazol-4-yl)pyrazolo[1,5-a]pyridin-5-yl)-6-phenyl-1H-pyrrolo[2,3-b]pyridine [2014];
6-phenyl-3-(3-(pyridin-3-yl)pyrazolo[1,5-a]pyridin-5-yl)-1H-pyrrolo[2,3-b]pyridine [2015];
N-(2,2-difluoroethyl)-5-(6-phenyl-1H-pyrrolo[2,3-b]pyridin-3-yl)pyrazolo[1,5-a]pyridine-3-carboxamide [2016];
(R)-5-(6-phenyl-1H-pyrrolo[2,3-b]pyridin-3-yl)-N-(1,1,1-trifluoropropan-2-yl)pyrazolo[1,5-a]pyridine-3-carboxamide [2017];
N-(1-methylpiperidin-4-yl)-5-(6-phenyl-1H-pyrrolo[2,3-b]pyridin-3-yl)pyrazolo[1,5-a]pyridine-3-carboxamide [2018];
5-(6-phenyl-1H-pyrrolo[2,3-b]pyridin-3-yl)-N-(pyridin-3-yl)pyrazolo[1,5-a]pyridine-3-carboxamide [2019];
(5-(6-phenyl-1H-pyrrolo[2,3-b]pyridin-3-yl)pyrazolo[1,5-a]pyridin-3-yl)(piperidin-1-yl)methanone [2020];
(4-methylpiperazin-1-yl)(5-(6-phenyl-1H-pyrrolo[2,3-b]pyridin-3-yl)pyrazolo[1,5-a]pyridin-3-yl)methanone [2021];
morpholino(5-(6-phenyl-1H-pyrrolo[2,3-b]pyridin-3-yl)pyrazolo[1,5-a]pyridin-3-yl)methanone [2022];
6-(6-phenyl-1H-pyrrolo[2,3-b]pyridin-3-yl)spiro[indene-1,4′-piperidin]-3(2H)-one [2024];
1′-methyl-6-(6-phenyl-1H-pyrrolo[2,3-b]pyridin-3-yl)spiro[indene-1,4′-piperidin]-3(2H)-one [2025];
5-(6-phenyl-1H-pyrrolo[2,3-b]pyridin-3-yl)isoindolin-1-one [2026];
3,3-dimethyl-5-(6-phenyl-1H-pyrrolo[2,3-b]pyridin-3-yl)isoindolin-1-one [2027];
6′-(6-phenyl-1H-pyrrolo[2,3-b]pyridin-3-yl)spiro[cyclohexane-1,1′-isoindolin]-3′-one [2028];
3-(chroman-6-yl)-6-phenyl-1H-pyrrolo[2,3-b]pyridine [2029];
6-(6-phenyl-1H-pyrrolo[2,3-b]pyridin-3-yl)-3,4-dihydroisoquinolin-1(2H)-one [2030];
4,4-dimethyl-6-(6-phenyl-1H-pyrrolo[2,3-b]pyridin-3-yl)-3,4-dihydroisoquinolin-1(2H)-one [2031];
7-(6-phenyl-1H-pyrrolo[2,3-b]pyridin-3-yl)spiro[chromane-2,4′-piperidin]-4-one [2032];
8-(6-phenyl-1H-pyrrolo[2,3-b]pyridin-3-yl)-3,4-dihydrobenzo[f][1,4]oxazepin-5(2H)-one [2033];
6-(6-phenyl-1H-pyrrolo[2,3-b]pyridin-3-yl)quinoline [2034];
6-(6-phenyl-1H-pyrrolo[2,3-b]pyridin-3-yl)quinoxaline [2035];
6-(6-phenyl-1H-pyrrolo[2,3-b]pyridin-3-yl)quinazoline [2036];
4-methoxy-6-(6-phenyl-1H-pyrrolo[2,3-b]pyridin-3-yl)quinazoline [2037];
4-(4-methylpiperazin-1-yl)-6-(6-phenyl-1H-pyrrolo[2,3-b]pyridin-3-yl)quinazoline [2038];
4-((1-methylpiperidin-4-yl)oxy)-6-(6-phenyl-1H-pyrrolo[2,3-b]pyridin-3-yl)quinazoline [2039];
N-(1-methylpiperidin-4-yl)-6-(6-phenyl-1H-pyrrolo[2,3-b]pyridin-3-yl)quinazolin-4-amine [2040];
6-(6-cyclobutyl-1H-pyrrolo[2,3-b]pyridin-3-yl)imidazo[1,2-a]pyrazine [2042];
4-fluoro-1-isopropyl-6-(6-isopropyl-1H-pyrrolo[2,3-b]pyridin-3-yl)-2-methyl-1H-benzo[d]imidazole [2045];
3-([1,2,4]triazolo[1,5-a]pyridin-6-yl)-N-(1-methylpiperidin-4-yl)-1H-pyrrolo[2,3-b]pyridine-6-carboxamide [2048];
N-((6-(4-methylpiperazin-1-yl)pyridin-3-yl)methyl)-3-(quinolin-6-yl)-1H-pyrrolo[2,3-b]pyridin-4-amine [2051];
N-((6-(1H-imidazol-1-yl)pyridin-3-yl)methyl)-3-(quinolin-6-yl)-1H-pyrrolo[2,3-b]pyridin-4-amine [2052];
6-(6-(cyclopropylmethoxy)-1H-pyrrolo[2,3-b]pyridin-3-yl)quinoline [2053];
N-(pyridin-4-yl)-3-(quinolin-6-yl)-1H-pyrrolo[2,3-b]pyridine-6-carboxamide [2054];
5-(3-(quinolin-6-yl)-1H-pyrrolo[2,3-b]pyridin-6-yl)thiazole [2055]; and
6-(6-cyclopropyl-1H-pyrrolo[2,3-b]pyridin-3-yl)-N-(2,2-difluoroethyl)-N-methylimidazo[1,2-a]pyridine-3-carboxamide [2056]; or a pharmaceutically acceptable salt thereof.

22. The compound according to claim 15, wherein the compound of Formula Ie is selected from the group consisting of:
5-(5-chloro-1H-pyrrolo[2,3-b]pyridin-3-yl)-2-methyl-2H-indazole [193];
2-(5-chloro-1H-pyrrolo[2,3-b]pyridin-3-yl)thieno[2,3-c]pyridine [194];
7-(5-chloro-1H-pyrrolo[2,3-b]pyridin-3-yl)-3,4-dihydropyrrolo[1,2-a]pyrazin-1(2H)-one [195];
6-(5-chloro-1H-pyrrolo[2,3-b]pyridin-3-yl)imidazo[1,2-a]pyridine [196];
6-(5-chloro-1H-pyrrolo[2,3-b]pyridin-3-yl)-N-(2,2-difluoroethyl)imidazo[1,2-a]pyridine-3-carboxamide [197];
6-(5-chloro-1H-pyrrolo[2,3-b]pyridin-3-yl)imidazo[1,2-a]pyrimidine [198];
6-(5-chloro-1H-pyrrolo[2,3-b]pyridin-3-yl)-4-fluoro-1-isopropyl-2-methyl-1H-benzo[d]imidazole [199];
6-(5-chloro-1H-pyrrolo[2,3-b]pyridin-3-yl)-4-fluoro-2-methyl-1-(1-methylpiperidin-4-yl)-1H-benzo[d]imidazole [200];
6-(5-chloro-1H-pyrrolo[2,3-b]pyridin-3-yl)-1-isopropyl-2-methyl-1H-imidazo[4,5-b]pyridine [201];
6-(5-chloro-1H-pyrrolo[2,3-b]pyridin-3-yl)-1-isopropyl-2-methyl-1H-imidazo[4,5-c]pyridine [202];
6-(5-chloro-1H-pyrrolo[2,3-b]pyridin-3-yl)-[1,2,4]triazolo[1,5-a]pyridine [203];
7-(5-chloro-1H-pyrrolo[2,3-b]pyridin-3-yl)-[1,2,4]triazolo[1,5-a]pyridine [204];
6-(5-chloro-1H-pyrrolo[2,3-b]pyridin-3-yl)-1-methyl-1H-benzo[d][1,2,3]triazole [205];
6-(5-chloro-1H-pyrrolo[2,3-b]pyridin-3-yl)imidazo[1,2-b]pyridazine [206];
5-chloro-3-(pyrazolo[1,5-a]pyridin-5-yl)-1H-pyrrolo[2,3-b]pyridine [207];
5-chloro-3-(3-(1-methyl-1H-pyrazol-4-yl)pyrazolo[1,5-a]pyridin-5-yl)-1H-pyrrolo[2,3-b]pyridine [208];
5-chloro-3-(3-(pyridin-3-yl)pyrazolo[1,5-a]pyridin-5-yl)-1H-pyrrolo[2,3-b]pyridine [209];
5-(5-chloro-1H-pyrrolo[2,3-b]pyridin-3-yl)-N-(2,2-difluoroethyl)pyrazolo[1,5-a]pyridine-3-carboxamide [210];
(R)-5-(5-chloro-1H-pyrrolo[2,3-b]pyridin-3-yl)-N-(1,1,1-trifluoropropan-2-yl)pyrazolo[1,5-a]pyridine-3-carboxamide [211];
5-(5-chloro-1H-pyrrolo[2,3-b]pyridin-3-yl)-N-(1-methylpiperidin-4-yl)pyrazolo[1,5-a]pyridine-3-carboxamide [212];
5-(5-chloro-1H-pyrrolo[2,3-b]pyridin-3-yl)-N-(pyridin-3-yl)pyrazolo[1,5-a]pyridine-3-carboxamide [213];
(5-(5-chloro-1H-pyrrolo[2,3-b]pyridin-3-yl)pyrazolo[1,5-a]pyridin-3-yl)(piperidin-1-yl)methanone [214];
(5-(5-chloro-1H-pyrrolo[2,3-b]pyridin-3-yl)pyrazolo[1,5-a]pyridin-3-yl)(4-methylpiperazin-1-yl)methanone [215];
(5-(5-chloro-1H-pyrrolo[2,3-b]pyridin-3-yl)pyrazolo[1,5-a]pyridin-3-yl)(morpholino)methanone [216];
6-(5-chloro-1H-pyrrolo[2,3-b]pyridin-3-yl)spiro[indene-1,4′-piperidin]-3(2H)-one [218];
6-(5-chloro-1H-pyrrolo[2,3-b]pyridin-3-yl)-1′-methylspiro[indene-1,4′-piperidin]-3(2H)-one [219];
5-(5-chloro-1H-pyrrolo[2,3-b]pyridin-3-yl)-3,3-dimethylisoindolin-1-one [221];
6′-(5-chloro-1H-pyrrolo[2,3-b]pyridin-3-yl)spiro[cyclohexane-1,1′-isoindolin]-3′-one [222];
5-chloro-3-(chroman-6-yl)-1H-pyrrolo[2,3-b]pyridine [223];
6-(5-chloro-1H-pyrrolo[2,3-b]pyridin-3-yl)-4,4-dimethyl-3,4-dihydroisoquinolin-1(2H)-one [225];
7-(5-chloro-1H-pyrrolo[2,3-b]pyridin-3-yl)spiro[chromane-2,4′-piperidin]-4-one [226];
8-(5-chloro-1H-pyrrolo[2,3-b]pyridin-3-yl)-3,4-dihydrobenzo[f][1,4]oxazepin-5(2H)-one [227];
6-(5-chloro-1H-pyrrolo[2,3-b]pyridin-3-yl)quinoxaline [229];
6-(5-chloro-1H-pyrrolo[2,3-b]pyridin-3-yl)quinazoline [230];
6-(5-chloro-1H-pyrrolo[2,3-b]pyridin-3-yl)-4-methoxyquinazoline [231];
6-(5-chloro-1H-pyrrolo[2,3-b]pyridin-3-yl)-4-(4-methylpiperazin-1-yl)quinazoline [232];
6-(5-chloro-1H-pyrrolo[2,3-b]pyridin-3-yl)-4-((1-methylpiperidin-4-yl)oxy)quinazoline [233];
6-(5-chloro-1H-pyrrolo[2,3-b]pyridin-3-yl)-N-(1-methylpiperidin-4-yl)quinazolin-4-amine [234];
5-(5-fluoro-1H-pyrrolo[2,3-b]pyridin-3-yl)-2-methyl-2H-indazole [235];
2-(5-fluoro-1H-pyrrolo[2,3-b]pyridin-3-yl)thieno[2,3-c]pyridine [236];
7-(5-fluoro-1H-pyrrolo[2,3-b]pyridin-3-yl)-3,4-dihydropyrrolo[1,2-a]pyrazin-1(2H)-one [237];
6-(5-fluoro-1H-pyrrolo[2,3-b]pyridin-3-yl)imidazo[1,2-a]pyridine [238];
6-(5-fluoro-1H-pyrrolo[2,3-b]pyridin-3-yl)-N-(2,2-difluoroethyl)imidazo[1,2-a]pyridine-3-carboxamide [239];
6-(5-fluoro-1H-pyrrolo[2,3-b]pyridin-3-yl)imidazo[1,2-a]pyrimidine [240];
6-(5-fluoro-1H-pyrrolo[2,3-b]pyridin-3-yl)-4-fluoro-1-isopropyl-2-methyl-1H-benzo[d]imidazole [241];
6-(5-fluoro-1H-pyrrolo[2,3-b]pyridin-3-yl)-4-fluoro-2-methyl-1-(1-methylpiperidin-4-yl)-1H-benzo[d]imidazole [242];
6-(5-fluoro-1H-pyrrolo[2,3-b]pyridin-3-yl)-1-isopropyl-2-methyl-1H-imidazo[4,5-b]pyridine [243];
6-(5-fluoro-1H-pyrrolo[2,3-b]pyridin-3-yl)-1-isopropyl-2-methyl-1H-imidazo[4,5-c]pyridine [244];
6-(5-fluoro-1H-pyrrolo[2,3-b]pyridin-3-yl)-[1,2,4]triazolo[1,5-a]pyridine [245];
7-(5-fluoro-1H-pyrrolo[2,3-b]pyridin-3-yl)-[1,2,4]triazolo[1,5-a]pyridine [246];
6-(5-fluoro-1H-pyrrolo[2,3-b]pyridin-3-yl)-1-methyl-1H-benzo[d][1,2,3]triazole [247];
6-(5-fluoro-1H-pyrrolo[2,3-b]pyridin-3-yl)imidazo[1,2-b]pyridazine [248];
5-fluoro-3-(pyrazolo[1,5-a]pyridin-5-yl)-1H-pyrrolo[2,3-b]pyridine [249];
5-fluoro-3-(3-(1-methyl-1H-pyrazol-4-yl)pyrazolo[1,5-a]pyridin-5-yl)-1H-pyrrolo[2,3-b]pyridine [250];
5-fluoro-3-(3-(pyridin-3-yl)pyrazolo[1,5-a]pyridin-5-yl)-1H-pyrrolo[2,3-b]pyridine [251];
5-(5-fluoro-1H-pyrrolo[2,3-b]pyridin-3-yl)-N-(2,2-difluoroethyl)pyrazolo[1,5-a]pyridine-3-carboxamide [252];
(R)-5-(5-fluoro-1H-pyrrolo[2,3-b]pyridin-3-yl)-N-(1,1,1-trifluoropropan-2-yl)pyrazolo[1,5-a]pyridine-3-carboxamide [253];
5-(5-fluoro-1H-pyrrolo[2,3-b]pyridin-3-yl)-N-(1-methylpiperidin-4-yl)pyrazolo[1,5-a]pyridine-3-carboxamide [254];
5-(5-fluoro-1H-pyrrolo[2,3-b]pyridin-3-yl)-N-(pyridin-3-yl)pyrazolo[1,5-a]pyridine-3-carboxamide [255];
(5-(5-fluoro-1H-pyrrolo[2,3-b]pyridin-3-yl)pyrazolo[1,5-a]pyridin-3-yl)(piperidin-1-yl)methanone [256];
(5-(5-fluoro-1H-pyrrolo[2,3-b]pyridin-3-yl)pyrazolo[1,5-a]pyridin-3-yl)(4-methylpiperazin-1-yl)methanone [257];
(5-(5-fluoro-1H-pyrrolo[2,3-b]pyridin-3-yl)pyrazolo[1,5-a]pyridin-3-yl)(morpholino)methanone [258];
6-(5-fluoro-1H-pyrrolo[2,3-b]pyridin-3-yl)spiro[indene-1,4′-piperidin]-3(2H)-one [260];
6-(5-fluoro-1H-pyrrolo[2,3-b]pyridin-3-yl)-1′-methylspiro[indene-1,4′-piperidin]-3(2H)-one [261];
5-(5-fluoro-1H-pyrrolo[2,3-b]pyridin-3-yl)-3,3-dimethylisoindolin-1-one [263];
6′-(5-fluoro-1H-pyrrolo[2,3-b]pyridin-3-yl)spiro[cyclohexane-1,1′-isoindolin]-3′-one [264];
5-fluoro-3-(chroman-6-yl)-1H-pyrrolo[2,3-b]pyridine [265];
6-(5-fluoro-1H-pyrrolo[2,3-b]pyridin-3-yl)-4,4-dimethyl-3,4-dihydroisoquinolin-1(2H)-one [267];
7-(5-fluoro-1H-pyrrolo[2,3-b]pyridin-3-yl)spiro[chromane-2,4′-piperidin]-4-one [268];
8-(5-fluoro-1H-pyrrolo[2,3-b]pyridin-3-yl)-3,4-dihydrobenzo[f][1,4]oxazepin-5(2H)-one [269];
6-(5-fluoro-1H-pyrrolo[2,3-b]pyridin-3-yl)quinoxaline [271];
6-(5-fluoro-1H-pyrrolo[2,3-b]pyridin-3-yl)quinazoline [272];
6-(5-fluoro-1H-pyrrolo[2,3-b]pyridin-3-yl)-4-methoxyquinazoline [273];
6-(5-fluoro-1H-pyrrolo[2,3-b]pyridin-3-yl)-4-(4-methylpiperazin-1-yl)quinazoline [274];
6-(5-fluoro-1H-pyrrolo[2,3-b]pyridin-3-yl)-4-((1-methylpiperidin-4-yl)oxy)quinazoline [275];
6-(5-fluoro-1H-pyrrolo[2,3-b]pyridin-3-yl)-N-(1-methylpiperidin-4-yl)quinazolin-4-amine [276];
2-methyl-5-(5-methyl-1H-pyrrolo[2,3-b]pyridin-3-yl)-2H-indazole [277];
2-(5-methyl-1H-pyrrolo[2,3-b]pyridin-3-yl)thieno[2,3-c]pyridine [278];
7-(5-methyl-1H-pyrrolo[2,3-b]pyridin-3-yl)-3,4-dihydropyrrolo[1,2-a]pyrazin-1(2H)-one [279];
6-(5-methyl-1H-pyrrolo[2,3-b]pyridin-3-yl)imidazo[1,2-a]pyridine [280];
N-(2,2-difluoroethyl)-6-(5-methyl-1H-pyrrolo[2,3-b]pyridin-3-yl)imidazo[1,2-a]pyridine-3-carboxamide [281];
6-(5-methyl-1H-pyrrolo[2,3-b]pyridin-3-yl)imidazo[1,2-a]pyrimidine [282];
4-fluoro-1-isopropyl-2-methyl-6-(5-methyl-1H-pyrrolo[2,3-b]pyridin-3-yl)-1H-benzo[d]imidazole [283];
4-fluoro-2-methyl-6-(5-methyl-1H-pyrrolo[2,3-b]pyridin-3-yl)-1-(1-methylpiperidin-4-yl)-1H-benzo[d]imidazole [284];
1-isopropyl-2-methyl-6-(5-methyl-1H-pyrrolo[2,3-b]pyridin-3-yl)-1H-imidazo[4,5-b]pyridine [285];
1-isopropyl-2-methyl-6-(5-methyl-1H-pyrrolo[2,3-b]pyridin-3-yl)-1H-imidazo[4,5-c]pyridine [286];
6-(5-methyl-1H-pyrrolo[2,3-b]pyridin-3-yl)-[1,2,4]triazolo[1,5-a]pyridine [287];
7-(5-methyl-1H-pyrrolo[2,3-b]pyridin-3-yl)-[1,2,4]triazolo[1,5-a]pyridine [288];
1-methyl-6-(5-methyl-1H-pyrrolo[2,3-b]pyridin-3-yl)-1H-benzo[d][1,2,3]triazole [289];
6-(5-methyl-1H-pyrrolo[2,3-b]pyridin-3-yl)imidazo[1,2-b]pyridazine [290];
5-methyl-3-(pyrazolo[1,5-a]pyridin-5-yl)-1H-pyrrolo[2,3-b]pyridine [291];
5-methyl-3-(3-(1-methyl-1H-pyrazol-4-yl)pyrazolo[1,5-a]pyridin-5-yl)-1H-pyrrolo[2,3-b]pyridine [292];
5-methyl-3-(3-(pyridin-3-yl)pyrazolo[1,5-a]pyridin-5-yl)-1H-pyrrolo[2,3-b]pyridine [293];
N-(2,2-difluoroethyl)-5-(5-methyl-1H-pyrrolo[2,3-b]pyridin-3-yl)pyrazolo[1,5-a]pyridine-3-carboxamide [294];
(R)-5-(5-methyl-1H-pyrrolo[2,3-b]pyridin-3-yl)-N-(1,1,1-trifluoropropan-2-yl)pyrazolo[1,5-a]pyridine-3-carboxamide [295];
5-(5-methyl-1H-pyrrolo[2,3-b]pyridin-3-yl)-N-(1-methylpiperidin-4-yl)pyrazolo[1,5-a]pyridine-3-carboxamide [296];
5-(5-methyl-1H-pyrrolo[2,3-b]pyridin-3-yl)-N-(pyridin-3-yl)pyrazolo[1,5-a]pyridine-3-carboxamide [297];
(5-(5-methyl-1H-pyrrolo[2,3-b]pyridin-3-yl)pyrazolo[1,5-a]pyridin-3-yl)(piperidin-1-yl)methanone [298];
(5-(5-methyl-1H-pyrrolo[2,3-b]pyridin-3-yl)pyrazolo[1,5-a]pyridin-3-yl)(4-methylpiperazin-1-yl)methanone [299];
(5-(5-methyl-1H-pyrrolo[2,3-b]pyridin-3-yl)pyrazolo[1,5-a]pyridin-3-yl)(morpholino)methanone [300];
6-(5-methyl-1H-pyrrolo[2,3-b]pyridin-3-yl)spiro[indene-1,4′-piperidin]-3(2H)-one [302];
1′-methyl-6-(5-methyl-1H-pyrrolo[2,3-b]pyridin-3-yl)spiro[indene-1,4′-piperidin]-3(2H)-one [303];
5-(5-methyl-1H-pyrrolo[2,3-b]pyridin-3-yl)isoindolin-1-one [304];
3,3-dimethyl-5-(5-methyl-1H-pyrrolo[2,3-b]pyridin-3-yl)isoindolin-1-one [305];
6′-(5-methyl-1H-pyrrolo[2,3-b]pyridin-3-yl)spiro[cyclohexane-1,1′-isoindolin]-3′-one [306];
3-(chroman-6-yl)-5-methyl-1H-pyrrolo[2,3-b]pyridine [307];
6-(5-methyl-1H-pyrrolo[2,3-b]pyridin-3-yl)-3,4-dihydroisoquinolin-1(2H)-one [308];
4,4-dimethyl-6-(5-methyl-1H-pyrrolo[2,3-b]pyridin-3-yl)-3,4-dihydroisoquinolin-1(2H)-one [309];
7-(5-methyl-1H-pyrrolo[2,3-b]pyridin-3-yl)spiro[chromane-2,4′-piperidin]-4-one [310];
8-(5-methyl-1H-pyrrolo[2,3-b]pyridin-3-yl)-3,4-dihydrobenzo[f][1,4]oxazepin-5(2H)-one [311];
6-(5-methyl-1H-pyrrolo[2,3-b]pyridin-3-yl)quinoxaline [313];
6-(5-methyl-1H-pyrrolo[2,3-b]pyridin-3-yl)quinazoline [314];
4-methoxy-6-(5-methyl-1H-pyrrolo[2,3-b]pyridin-3-yl)quinazoline [315];
6-(5-methyl-1H-pyrrolo[2,3-b]pyridin-3-yl)-4-(4-methylpiperazin-1-yl)quinazoline [316];
6-(5-methyl-1H-pyrrolo[2,3-b]pyridin-3-yl)-4-((1-methylpiperidin-4-yl)oxy)quinazoline [317];
6-(5-methyl-1H-pyrrolo[2,3-b]pyridin-3-yl)-N-(1-methylpiperidin-4-yl)quinazolin-4-amine [318];
2-methyl-5-(5-(1-methyl-1,2,3,6-tetrahydropyridin-4-yl)-1H-pyrrolo[2,3-b]pyridin-3-yl)-2H-indazole [319];
2-(5-(1-methyl-1,2,3,6-tetrahydropyridin-4-yl)-1H-pyrrolo[2,3-b]pyridin-3-yl)thieno[2,3-c]pyridine [320];
7-(5-(1-methyl-1,2,3,6-tetrahydropyridin-4-yl)-1H-pyrrolo[2,3-b]pyridin-3-yl)-3,4-dihydropyrrolo[1,2-a]pyrazin-1(2H)-one [321];
6-(5-(1-methyl-1,2,3,6-tetrahydropyridin-4-yl)-1H-pyrrolo[2,3-b]pyridin-3-yl)imidazo[1,2-a]pyridine [322];
N-(2,2-difluoroethyl)-6-(5-(1-methyl-1,2,3,6-tetrahydropyridin-4-yl)-1H-pyrrolo[2,3-b]pyridin-3-yl)imidazo[1,2-a]pyridine-3-carboxamide [323];
6-(5-(1-methyl-1,2,3,6-tetrahydropyridin-4-yl)-1H-pyrrolo[2,3-b]pyridin-3-yl)imidazo[1,2-a]pyrimidine [324];
4-fluoro-1-isopropyl-2-methyl-6-(5-(1-methyl-1,2,3,6-tetrahydropyridin-4-yl)-1H-pyrrolo[2,3-b]pyridin-3-yl)-1H-benzo[d]imidazole [325];
4-fluoro-2-methyl-6-(5-(1-methyl-1,2,3,6-tetrahydropyridin-4-yl)-1H-pyrrolo[2,3-b]pyridin-3-yl)-1-(1-methylpiperidin-4-yl)-1H-benzo[d]imidazole [326];
1-isopropyl-2-methyl-6-(5-(1-methyl-1,2,3,6-tetrahydropyridin-4-yl)-1H-pyrrolo[2,3-b]pyridin-3-yl)-1H-imidazo[4,5-b]pyridine [327];
1-isopropyl-2-methyl-6-(5-(1-methyl-1,2,3,6-tetrahydropyridin-4-yl)-1H-pyrrolo[2,3-b]pyridin-3-yl)-1H-imidazo[4,5-c]pyridine [328];
6-(5-(1-methyl-1,2,3,6-tetrahydropyridin-4-yl)-1H-pyrrolo[2,3-b]pyridin-3-yl)-[1,2,4]triazolo[1,5-a]pyridine [329];
7-(5-(1-methyl-1,2,3,6-tetrahydropyridin-4-yl)-1H-pyrrolo[2,3-b]pyridin-3-yl)-[1,2,4]triazolo[1,5-a]pyridine [330];
1-methyl-6-(5-(1-methyl-1,2,3,6-tetrahydropyridin-4-yl)-1H-pyrrolo[2,3-b]pyridin-3-yl)-1H-benzo[d][1,2,3]triazole [331];
6-(5-(1-methyl-1,2,3,6-tetrahydropyridin-4-yl)-1H-pyrrolo[2,3-b]pyridin-3-yl)imidazo[1,2-b]pyridazine [332];
5-(1-methyl-1,2,3,6-tetrahydropyridin-4-yl)-3-(pyrazolo[1,5-a]pyridin-5-yl)-1H-pyrrolo[2,3-b]pyridine [333];
5-(1-methyl-1,2,3,6-tetrahydropyridin-4-yl)-3-(3-(1-methyl-1H-pyrazol-4-yl)pyrazolo[1,5-a]pyridin-5-yl)-1H-pyrrolo[2,3-b]pyridine [334];
5-(1-methyl-1,2,3,6-tetrahydropyridin-4-yl)-3-(3-(pyridin-3-yl)pyrazolo[1,5-a]pyridin-5-yl)-1H-pyrrolo[2,3-b]pyridine [335];
N-(2,2-difluoroethyl)-5-(5-(1-methyl-1,2,3,6-tetrahydropyridin-4-yl)-1H-pyrrolo[2,3-b]pyridin-3-yl)pyrazolo[1,5-a]pyridine-3-carboxamide [336];
(R)-5-(5-(1-methyl-1,2,3,6-tetrahydropyridin-4-yl)-1H-pyrrolo[2,3-b]pyridin-3-yl)-N-(1,1,1-trifluoropropan-2-yl)pyrazolo[1,5-a]pyridine-3-carboxamide [337];
5-(5-(1-methyl-1,2,3,6-tetrahydropyridin-4-yl)-1H-pyrrolo[2,3-b]pyridin-3-yl)-N-(1-methylpiperidin-4-yl)pyrazolo[1,5-a]pyridine-3-carboxamide [338];
5-(5-(1-methyl-1,2,3,6-tetrahydropyridin-4-yl)-1H-pyrrolo[2,3-b]pyridin-3-yl)-N-(pyridin-3-yl)pyrazolo[1,5-a]pyridine-3-carboxamide [339];
(5-(5-(1-methyl-1,2,3,6-tetrahydropyridin-4-yl)-1H-pyrrolo[2,3-b]pyridin-3-yl)pyrazolo[1,5-a]pyridin-3-yl)(piperidin-1-yl)methanone [340];
(5-(5-(1-methyl-1,2,3,6-tetrahydropyridin-4-yl)-1H-pyrrolo[2,3-b]pyridin-3-yl)pyrazolo[1,5-a]pyridin-3-yl)(4-methylpiperazin-1-yl)methanone [341];
(5-(5-(1-methyl-1,2,3,6-tetrahydropyridin-4-yl)-1H-pyrrolo[2,3-b]pyridin-3-yl)pyrazolo[1,5-a]pyridin-3-yl)(morpholino)methanone [342];
6-(5-(1-methyl-1,2,3,6-tetrahydropyridin-4-yl)-1H-pyrrolo[2,3-b]pyridin-3-yl)spiro[indene-1,4′-piperidin]-3(2H)-one [344];
1′-methyl-6-(5-(1-methyl-1,2,3,6-tetrahydropyridin-4-yl)-1H-pyrrolo[2,3-b]pyridin-3-yl)spiro[indene-1,4′-piperidin]-3(2H)-one [345];
5-(5-(1-methyl-1,2,3,6-tetrahydropyridin-4-yl)-1H-pyrrolo[2,3-b]pyridin-3-yl)isoindolin-1-one [346];
3,3-dimethyl-5-(5-(1-methyl-1,2,3,6-tetrahydropyridin-4-yl)-1H-pyrrolo[2,3-b]pyridin-3-yl)isoindolin-1-one [347];
6′-(5-(1-methyl-1,2,3,6-tetrahydropyridin-4-yl)-1H-pyrrolo[2,3-b]pyridin-3-yl)spiro[cyclohexane-1,1′-isoindolin]-3′-one [348];
3-(chroman-6-yl)-5-(1-methyl-1,2,3,6-tetrahydropyridin-4-yl)-1H-pyrrolo[2,3-b]pyridine [349];
6-(5-(1-methyl-1,2,3,6-tetrahydropyridin-4-yl)-1H-pyrrolo[2,3-b]pyridin-3-yl)-3,4-dihydroisoquinolin-1(2H)-one [350];
4,4-dimethyl-6-(5-(1-methyl-1,2,3,6-tetrahydropyridin-4-yl)-1H-pyrrolo[2,3-b]pyridin-3-yl)-3,4-dihydroisoquinolin-1(2H)-one [351];
7-(5-(1-methyl-1,2,3,6-tetrahydropyridin-4-yl)-1H-pyrrolo[2,3-b]pyridin-3-yl)spiro[chromane-2,4′-piperidin]-4-one [352];
8-(5-(1-methyl-1,2,3,6-tetrahydropyridin-4-yl)-1H-pyrrolo[2,3-b]pyridin-3-yl)-3,4-dihydrobenzo[f][1,4]oxazepin-5(2H)-one [353];
6-(5-(1-methyl-1,2,3,6-tetrahydropyridin-4-yl)-1H-pyrrolo[2,3-b]pyridin-3-yl)quinoline [354];
6-(5-(1-methyl-1,2,3,6-tetrahydropyridin-4-yl)-1H-pyrrolo[2,3-b]pyridin-3-yl)quinoxaline [355];
6-(5-(1-methyl-1,2,3,6-tetrahydropyridin-4-yl)-1H-pyrrolo[2,3-b]pyridin-3-yl)quinazoline [356];
4-methoxy-6-(5-(1-methyl-1,2,3,6-tetrahydropyridin-4-yl)-1H-pyrrolo[2,3-b]pyridin-3-yl)quinazoline [357];
6-(5-(1-methyl-1,2,3,6-tetrahydropyridin-4-yl)-1H-pyrrolo[2,3-b]pyridin-3-yl)-4-(4-methylpiperazin-1-yl)quinazoline [358];
6-(5-(1-methyl-1,2,3,6-tetrahydropyridin-4-yl)-1H-pyrrolo[2,3-b]pyridin-3-yl)-4-((1-methylpiperidin-4-yl)oxy)quinazoline [359];
6-(5-(1-methyl-1,2,3,6-tetrahydropyridin-4-yl)-1H-pyrrolo[2,3-b]pyridin-3-yl)-N-(1-methylpiperidin-4-yl)quinazolin-4-amine [360];
2-methyl-5-(5-(4-methylpiperazin-1-yl)-1H-pyrrolo[2,3-b]pyridin-3-yl)-2H-indazole [361];
2-(5-(4-methylpiperazin-1-yl)-1H-pyrrolo[2,3-b]pyridin-3-yl)thieno[2,3-c]pyridine [362];
7-(5-(4-methylpiperazin-1-yl)-1H-pyrrolo[2,3-b]pyridin-3-yl)-3,4-dihydropyrrolo[1,2-a]pyrazin-1(2H)-one [363];
6-(5-(4-methylpiperazin-1-yl)-1H-pyrrolo[2,3-b]pyridin-3-yl)imidazo[1,2-a]pyridine [364];
N-(2,2-difluoroethyl)-6-(5-(4-methylpiperazin-1-yl)-1H-pyrrolo[2,3-b]pyridin-3-yl)imidazo[1,2-a]pyridine-3-carboxamide [365];
6-(5-(4-methylpiperazin-1-yl)-1H-pyrrolo[2,3-b]pyridin-3-yl)imidazo[1,2-a]pyrimidine [366];
4-fluoro-1-isopropyl-2-methyl-6-(5-(4-methylpiperazin-1-yl)-1H-pyrrolo[2,3-b]pyridin-3-yl)-1H-benzo[d]imidazole [367];
4-fluoro-2-methyl-6-(5-(4-methylpiperazin-1-yl)-1H-pyrrolo[2,3-b]pyridin-3-yl)-1-(1-methylpiperidin-4-yl)-1H-benzo[d]imidazole [368];
1-isopropyl-2-methyl-6-(5-(4-methylpiperazin-1-yl)-1H-pyrrolo[2,3-b]pyridin-3-yl)-1H-imidazo[4,5-b]pyridine [369];
1-isopropyl-2-methyl-6-(5-(4-methylpiperazin-1-yl)-1H-pyrrolo[2,3-b]pyridin-3-yl)-1H-imidazo[4,5-c]pyridine [370];
6-(5-(4-methylpiperazin-1-yl)-1H-pyrrolo[2,3-b]pyridin-3-yl)-[1,2,4]triazolo[1,5-a]pyridine [371];
7-(5-(4-methylpiperazin-1-yl)-1H-pyrrolo[2,3-b]pyridin-3-yl)-[1,2,4]triazolo[1,5-a]pyridine [372];
1-methyl-6-(5-(4-methylpiperazin-1-yl)-1H-pyrrolo[2,3-b]pyridin-3-yl)-1H-benzo[d][1,2,3]triazole [373];
6-(5-(4-methylpiperazin-1-yl)-1H-pyrrolo[2,3-b]pyridin-3-yl)imidazo[1,2-b]pyridazine [374];
5-(4-methylpiperazin-1-yl)-3-(pyrazolo[1,5-a]pyridin-5-yl)-1H-pyrrolo[2,3-b]pyridine [375];
3-(3-(1-methyl-1H-pyrazol-4-yl)pyrazolo[1,5-a]pyridin-5-yl)-5-(4-methylpiperazin-1-yl)-1H-pyrrolo[2,3-1)]pyridine [376];
5-(4-methylpiperazin-1-yl)-3-(3-(pyridin-3-yl)pyrazolo[1,5-a]pyridin-5-yl)-1H-pyrrolo[2,3-b]pyridine [377];
N-(2,2-difluoroethyl)-5-(5-(4-methylpiperazin-1-yl)-1H-pyrrolo[2,3-b]pyridin-3-yl)pyrazolo[1,5-a]pyridine-3-carboxamide [378];
(R)-5-(5-(4-methylpiperazin-1-yl)-1H-pyrrolo[2,3-b]pyridin-3-yl)-N-(1,1,1-trifluoropropan-2-yl)pyrazolo[1,5-a]pyridine-3-carboxamide [379];
5-(5-(4-methylpiperazin-1-yl)-1H-pyrrolo[2,3-b]pyridin-3-yl)-N-(1-methylpiperidin-4-yl)pyrazolo[1,5-a]pyridine-3-carboxamide [380];
5-(5-(4-methylpiperazin-1-yl)-1H-pyrrolo[2,3-b]pyridin-3-yl)-N-(pyridin-3-yl)pyrazolo[1,5-a]pyridine-3-carboxamide [381];
(5-(5-(4-methylpiperazin-1-yl)-1H-pyrrolo[2,3-b]pyridin-3-yl)pyrazolo[1,5-a]pyridin-3-yl)(piperidin-1-yl)methanone [382];
(4-methylpiperazin-1-yl)(5-(5-(4-methylpiperazin-1-yl)-1H-pyrrolo[2,3-b]pyridin-3-yl)pyrazolo[1,5-a]pyridin-3-yl)methanone [383];
(5-(5-(4-methylpiperazin-1-yl)-1H-pyrrolo[2,3-b]pyridin-3-yl)pyrazolo[1,5-a]pyridin-3-yl)(morpholino)methanone [384];
6-(5-(4-methylpiperazin-1-yl)-1H-pyrrolo[2,3-b]pyridin-3-yl)spiro[indene-1,4′-piperidin]-3(2H)-one [386];
1′-methyl-6-(5-(4-methylpiperazin-1-yl)-1H-pyrrolo[2,3-b]pyridin-3-yl)spiro[indene-1,4′-piperidin]-3(2H)-one [387];
5-(5-(4-methylpiperazin-1-yl)-1H-pyrrolo[2,3-b]pyridin-3-yl)isoindolin-1-one [388];
3,3-dimethyl-5-(5-(4-methylpiperazin-1-yl)-1H-pyrrolo[2,3-b]pyridin-3-yl)isoindolin-1-one [389];
6′-(5-(4-methylpiperazin-1-yl)-1H-pyrrolo[2,3-b]pyridin-3-yl)spiro[cyclohexane-1,1′-isoindolin]-3′-one [390];
3-(chroman-6-yl)-5-(4-methylpiperazin-1-yl)-1H-pyrrolo[2,3-b]pyridine [391];
6-(5-(4-methylpiperazin-1-yl)-1H-pyrrolo[2,3-b]pyridin-3-yl)-3,4-dihydroisoquinolin-1(2H)-one [392];
4,4-dimethyl-6-(5-(4-methylpiperazin-1-yl)-1H-pyrrolo[2,3-b]pyridin-3-yl)-3,4-dihydroisoquinolin-1(2H)-one [393];
7-(5-(4-methylpiperazin-1-yl)-1H-pyrrolo[2,3-b]pyridin-3-yl)spiro[chromane-2,4′-piperidin]-4-one [394];
8-(5-(4-methylpiperazin-1-yl)-1H-pyrrolo[2,3-b]pyridin-3-yl)-3,4-dihydrobenzo[f][1,4]oxazepin-5(2H)-one [395];
6-(5-(4-methylpiperazin-1-yl)-1H-pyrrolo[2,3-b]pyridin-3-yl)quinoline [396];
6-(5-(4-methylpiperazin-1-yl)-1H-pyrrolo[2,3-b]pyridin-3-yl)quinoxaline [397];
6-(5-(4-methylpiperazin-1-yl)-1H-pyrrolo[2,3-b]pyridin-3-yl)quinazoline [398];
4-methoxy-6-(5-(4-methylpiperazin-1-yl)-1H-pyrrolo[2,3-b]pyridin-3-yl)quinazoline [399];
4-(4-methylpiperazin-1-yl)-6-(5-(4-methylpiperazin-1-yl)-1H-pyrrolo[2,3-b]pyridin-3-yl)quinazoline [400];
6-(5-(4-methylpiperazin-1-yl)-1H-pyrrolo[2,3-b]pyridin-3-yl)-4-((1-methylpiperidin-4-yl)oxy)quinazoline [401];
6-(5-(4-methylpiperazin-1-yl)-1H-pyrrolo[2,3-b]pyridin-3-yl)-N-(1-methylpiperidin-4-yl)quinazolin-4-amine [402];
2-methyl-5-(5-((4-methylpiperazin-1-yl)methyl)-1H-pyrrolo[2,3-b]pyridin-3-yl)-2H-indazole [403];
2-(5-((4-methylpiperazin-1-yl)methyl)-1H-pyrrolo[2,3-b]pyridin-3-yl)thieno[2,3-c]pyridine [404];
7-(5-((4-methylpiperazin-1-yl)methyl)-1H-pyrrolo[2,3-b]pyridin-3-yl)-3,4-dihydropyrrolo[1,2-a]pyrazin-1(2H)-one [405];
6-(5-((4-methylpiperazin-1-yl)methyl)-1H-pyrrolo[2,3-b]pyridin-3-yl)imidazo[1,2-a]pyridine [406];
N-(2,2-difluoroethyl)-6-(5-((4-methylpiperazin-1-yl)methyl)-1H-pyrrolo[2,3-b]pyridin-3-yl)imidazo[1,2-a]pyridine-3-carboxamide [407];
6-(5-((4-methylpiperazin-1-yl)methyl)-1H-pyrrolo[2,3-b]pyridin-3-yl)imidazo[1,2-a]pyrimidine [408];
4-fluoro-1-isopropyl-2-methyl-6-(5-((4-methylpiperazin-1-yl)methyl)-1H-pyrrolo[2,3-b]pyridin-3-yl)-1H-benzo[d]imidazole [409];
4-fluoro-2-methyl-6-(5-((4-methylpiperazin-1-yl)methyl)-1H-pyrrolo[2,3-b]pyridin-3-yl)-1-(1-methylpiperidin-4-yl)-1H-benzo[d]imidazole [410];
1-isopropyl-2-methyl-6-(5-((4-methylpiperazin-1-yl)methyl)-1H-pyrrolo[2,3-b]pyridin-3-yl)-1H-imidazo[4,5-b]pyridine [411];
1-isopropyl-2-methyl-6-(5-((4-methylpiperazin-1-yl)methyl)-1H-pyrrolo[2,3-b]pyridin-3-yl)-1H-imidazo[4,5-c]pyridine [412];
6-(5-((4-methylpiperazin-1-yl)methyl)-1H-pyrrolo[2,3-b]pyridin-3-yl)-[1,2,4]triazolo[1,5-a]pyridine [413];
7-(5-((4-methylpiperazin-1-yl)methyl)-1H-pyrrolo[2,3-b]pyridin-3-yl)-[1,2,4]triazolo[1,5-a]pyridine [414];
1-methyl-6-(5-((4-methylpiperazin-1-yl)methyl)-1H-pyrrolo[2,3-b]pyridin-3-yl)-1H-benzo[d][1,2,3]triazole [415];
6-(5-((4-methylpiperazin-1-yl)methyl)-1H-pyrrolo[2,3-b]pyridin-3-yl)imidazo[1,2-b]pyridazine [416];
5-((4-methylpiperazin-1-yl)methyl)-3-(pyrazolo[1,5-a]pyridin-5-yl)-1H-pyrrolo[2,3-1)]pyridine [417];
3-(3-(1-methyl-1H-pyrazol-4-yl)pyrazolo[1,5-a]pyridin-5-yl)-5-((4-methylpiperazin-1-yl)methyl)-1H-pyrrolo[2,3-1)]pyridine [418];
5-((4-methylpiperazin-1-yl)methyl)-3-(3-(pyridin-3-yl)pyrazolo[1,5-a]pyridin-5-yl)-1H-pyrrolo[2,3-1)]pyridine [419];
N-(2,2-difluoroethyl)-5-(5-((4-methylpiperazin-1-yl)methyl)-1H-pyrrolo[2,3-b]pyridin-3-yl)pyrazolo[1,5-a]pyridine-3-carboxamide [420];
(R)-5-(5-((4-methylpiperazin-1-yl)methyl)-1H-pyrrolo[2,3-b]pyridin-3-yl)-N-(1,1,1-trifluoropropan-2-yl)pyrazolo[1,5-a]pyridine-3-carboxamide [421];
5-(5-((4-methylpiperazin-1-yl)methyl)-1H-pyrrolo[2,3-b]pyridin-3-yl)-N-(1-methylpiperidin-4-yl)pyrazolo[1,5-a]pyridine-3-carboxamide [422];
5-(5-((4-methylpiperazin-1-yl)methyl)-1H-pyrrolo[2,3-b]pyridin-3-yl)-N-(pyridin-3-yl)pyrazolo[1,5-a]pyridine-3-carboxamide [423];
(5-(5-((4-methylpiperazin-1-yl)methyl)-1H-pyrrolo[2,3-b]pyridin-3-yl)pyrazolo[1,5-a]pyridin-3-yl)(piperidin-1-yl)methanone [424];
(4-methylpiperazin-1-yl)(5-(5-((4-methylpiperazin-1-yl)methyl)-1H-pyrrolo[2,3-b]pyridin-3-yl)pyrazolo[1,5-a]pyridin-3-yl)methanone [425];
(5-(5-((4-methylpiperazin-1-yl)methyl)-1H-pyrrolo[2,3-b]pyridin-3-yl)pyrazolo[1,5-a]pyridin-3-yl)(morpholino)methanone [426];
6-(5-((4-methylpiperazin-1-yl)methyl)-1H-pyrrolo[2,3-b]pyridin-3-yl)spiro[indene-1,4′-piperidin]-3(2H)-one [428];
1′-methyl-6-(5-((4-methylpiperazin-1-yl)methyl)-1H-pyrrolo[2,3-b]pyridin-3-yl)spiro[indene-1,4′-piperidin]-3(2H)-one [429];
5-(5-((4-methylpiperazin-1-yl)methyl)-1H-pyrrolo[2,3-b]pyridin-3-yl)isoindolin-1-one [430];
3,3-dimethyl-5-(5-((4-methylpiperazin-1-yl)methyl)-1H-pyrrolo[2,3-b]pyridin-3-yl)isoindolin-1-one [431];
6′-(5-((4-methylpiperazin-1-yl)methyl)-1H-pyrrolo[2,3-b]pyridin-3-yl)spiro[cyclohexane-1,1′-isoindolin]-3′-one [432];
3-(chroman-6-yl)-5-((4-methylpiperazin-1-yl)methyl)-1H-pyrrolo[2,3-b]pyridine [433];
6-(5-((4-methylpiperazin-1-yl)methyl)-1H-pyrrolo[2,3-b]pyridin-3-yl)-3,4-dihydroisoquinolin-1(2H)-one [434];
4,4-dimethyl-6-(5-((4-methylpiperazin-1-yl)methyl)-1H-pyrrolo[2,3-b]pyridin-3-yl)-3,4-dihydroisoquinolin-1(2H)-one [435];
7-(5-((4-methylpiperazin-1-yl)methyl)-1H-pyrrolo[2,3-b]pyridin-3-yl)spiro[chromane-2,4′-piperidin]-4-one [436];
8-(5-((4-methylpiperazin-1-yl)methyl)-1H-pyrrolo[2,3-b]pyridin-3-yl)-3,4-dihydrobenzo[f][1,4]oxazepin-5(2H)-one [437];
6-(5-((4-methylpiperazin-1-yl)methyl)-1H-pyrrolo[2,3-b]pyridin-3-yl)quinoline [438];
6-(5-((4-methylpiperazin-1-yl)methyl)-1H-pyrrolo[2,3-b]pyridin-3-yl)quinoxaline [439];
6-(5-((4-methylpiperazin-1-yl)methyl)-1H-pyrrolo[2,3-b]pyridin-3-yl)quinazoline [440];
4-methoxy-6-(5-((4-methylpiperazin-1-yl)methyl)-1H-pyrrolo[2,3-b]pyridin-3-yl)quinazoline [441];
4-(4-methylpiperazin-1-yl)-6-(5-((4-methylpiperazin-1-yl)methyl)-1H-pyrrolo[2,3-b]pyridin-3-yl)quinazoline [442];
6-(5-((4-methylpiperazin-1-yl)methyl)-1H-pyrrolo[2,3-b]pyridin-3-yl)-4-((1-methylpiperidin-4-yl)oxy)quinazoline [443];
6-(5-((4-methylpiperazin-1-yl)methyl)-1H-pyrrolo[2,3-b]pyridin-3-yl)-N-(1-methylpiperidin-4-yl)quinazolin-4-amine [444];
3-(2-methyl-2H-indazol-5-yl)-N-(1-methylpiperidin-4-yl)-1H-pyrrolo[2,3-b]pyridine-5-carboxamide [445];
N-(1-methylpiperidin-4-yl)-3-(thieno[2,3-c]pyridin-2-yl)-1H-pyrrolo[2,3-b]pyridine-5-carboxamide [446];
N-(1-methylpiperidin-4-yl)-3-(1-oxo-1,2,3,4-tetrahydropyrrolo[1,2-a]pyrazin-7-yl)-1H-pyrrolo[2,3-b]pyridine-5-carboxamide [447];
3-(imidazo[1,2-a]pyridin-6-yl)-N-(1-methylpiperidin-4-yl)-1H-pyrrolo[2,3-b]pyridine-5-carboxamide [448];
N-(2,2-difluoroethyl)-6-(5-((1-methylpiperidin-4-yl)carbamoyl)-1H-pyrrolo[2,3-b]pyridin-3-yl)imidazo[1,2-a]pyridine-3-carboxamide [449];
3-(imidazo[1,2-a]pyrimidin-6-yl)-N-(1-methylpiperidin-4-yl)-1H-pyrrolo[2,3-b]pyridine-5-carboxamide [450];
3-(4-fluoro-1-isopropyl-2-methyl-1H-benzo[d]imidazol-6-yl)-N-(1-methylpiperidin-4-yl)-1H-pyrrolo[2,3-b]pyridine-5-carboxamide [451];
3-(4-fluoro-2-methyl-1-(1-methylpiperidin-4-yl)-1H-benzo[d]imidazol-6-yl)-N-(1-methylpiperidin-4-yl)-1H-pyrrolo[2,3-b]pyridine-5-carboxamide [452];
3-(1-isopropyl-2-methyl-1H-imidazo[4,5-b]pyridin-6-yl)-N-(1-methylpiperidin-4-yl)-1H-pyrrolo[2,3-b]pyridine-5-carboxamide [453];
3-(1-isopropyl-2-methyl-1H-imidazo[4,5-c]pyridin-6-yl)-N-(1-methylpiperidin-4-yl)-1H-pyrrolo[2,3-b]pyridine-5-carboxamide [454];
3-([1,2,4]triazolo[1,5-a]pyridin-6-yl)-N-(1-methylpiperidin-4-yl)-1H-pyrrolo[2,3-b]pyridine-5-carboxamide [455];
3-([1,2,4]triazolo[1,5-a]pyridin-7-yl)-N-(1-methylpiperidin-4-yl)-1H-pyrrolo[2,3-b]pyridine-5-carboxamide [456];
3-(1-methyl-1H-benzo[d][1,2,3]triazol-6-yl)-N-(1-methylpiperidin-4-yl)-1H-pyrrolo[2,3-b]pyridine-5-carboxamide [457];
3-(imidazo[1,2-b]pyridazin-6-yl)-N-(1-methylpiperidin-4-yl)-1H-pyrrolo[2,3-b]pyridine-5-carboxamide [458];
N-(1-methylpiperidin-4-yl)-3-(pyrazolo[1,5-a]pyridin-5-yl)-1H-pyrrolo[2,3-b]pyridine-5-carboxamide [459];
3-(3-(1-methyl-1H-pyrazol-4-yl)pyrazolo[1,5-a]pyridin-5-yl)-N-(1-methylpiperidin-4-yl)-1H-pyrrolo[2,3-b]pyridine-5-carboxamide [460];
N-(1-methylpiperidin-4-yl)-3-(3-(pyridin-3-yl)pyrazolo[1,5-a]pyridin-5-yl)-1H-pyrrolo[2,3-b]pyridine-5-carboxamide [461];
3-(3-((2,2-difluoroethyl)carbamoyl)pyrazolo[1,5-a]pyridin-5-yl)-N-(1-methylpiperidin-4-yl)-1H-pyrrolo[2,3-b]pyridine-5-carboxamide [462];
(R)-N-(1-methylpiperidin-4-yl)-3-(3-((1,1,1-trifluoropropan-2-yl)carbamoyl)pyrazolo[1,5-a]pyridin-5-yl)-1H-pyrrolo[2,3-b]pyridine-5-carboxamide [463];
N-(1-methylpiperidin-4-yl)-3-(3-((1-methylpiperidin-4-yl)carbamoyl)pyrazolo[1,5-a]pyridin-5-yl)-1H-pyrrolo[2,3-b]pyridine-5-carboxamide [464];
N-(1-methylpiperidin-4-yl)-3-(3-(pyridin-3-ylcarbamoyl)pyrazolo[1,5-a]pyridin-5-yl)-1H-pyrrolo[2,3-b]pyridine-5-carboxamide [465];
N-(1-methylpiperidin-4-yl)-3-(3-(piperidine-1-carbonyl)pyrazolo[1,5-a]pyridin-5-yl)-1H-pyrrolo[2,3-b]pyridine-5-carboxamide [466];
3-(3-(4-methylpiperazine-1-carbonyl)pyrazolo[1,5-a]pyridin-5-yl)-N-(1-methylpiperidin-4-yl)-1H-pyrrolo[2,3-b]pyridine-5-carboxamide [467];
N-(1-methylpiperidin-4-yl)-3-(3-(morpholine-4-carbonyl)pyrazolo[1,5-a]pyridin-5-yl)-1H-pyrrolo[2,3-b]pyridine-5-carboxamide [468];
N-(1-methylpiperidin-4-yl)-3-(3-oxo-2,3-dihydrospiro[indene-1,4′-piperidin]-6-yl)-1H-pyrrolo[2,3-b]pyridine-5-carboxamide [470];
3-(1′-methyl-3-oxo-2,3-dihydrospiro[indene-1,4′-piperidin]-6-yl)-N-(1-methylpiperidin-4-yl)-1H-pyrrolo[2,3-b]pyridine-5-carboxamide [471];
N-(1-methylpiperidin-4-yl)-3-(1-oxoisoindolin-5-yl)-1H-pyrrolo[2,3-b]pyridine-5-carboxamide [472];
3-(3,3-dimethyl-1-oxoisoindolin-5-yl)-N-(1-methylpiperidin-4-yl)-1H-pyrrolo[2,3-b]pyridine-5-carboxamide [473];
N-(1-methylpiperidin-4-yl)-3-(3′-oxospiro[cyclohexane-1,1′-isoindolin]-6′-yl)-1H-pyrrolo[2,3-b]pyridine-5-carboxamide [474];
3-(chroman-6-yl)-N-(1-methylpiperidin-4-yl)-1H-pyrrolo[2,3-b]pyridine-5-carboxamide [475];
N-(1-methylpiperidin-4-yl)-3-(1-oxo-1,2,3,4-tetrahydroisoquinolin-6-yl)-1H-pyrrolo[2,3-b]pyridine-5-carboxamide [476];
3-(4,4-dimethyl-1-oxo-1,2,3,4-tetrahydroisoquinolin-6-yl)-N-(1-methylpiperidin-4-yl)-1H-pyrrolo[2,3-b]pyridine-5-carboxamide [477];
N-(1-methylpiperidin-4-yl)-3-(4-oxospiro[chromane-2,4′-piperidin]-7-yl)-1H-pyrrolo[2,3-b]pyridine-5-carboxamide [478];
N-(1-methylpiperidin-4-yl)-3-(5-oxo-2,3,4,5-tetrahydrobenzo[f][1,4]oxazepin-8-yl)-1H-pyrrolo[2,3-b]pyridine-5-carboxamide [479];
N-(1-methylpiperidin-4-yl)-3-(quinolin-6-yl)-1H-pyrrolo[2,3-b]pyridine-5-carboxamide [480];
N-(1-methylpiperidin-4-yl)-3-(quinoxalin-6-yl)-1H-pyrrolo[2,3-b]pyridine-5-carboxamide [481];
N-(1-methylpiperidin-4-yl)-3-(quinazolin-6-yl)-1H-pyrrolo[2,3-b]pyridine-5-carboxamide [482];
3-(4-methoxyquinazolin-6-yl)-N-(1-methylpiperidin-4-yl)-1H-pyrrolo[2,3-b]pyridine-5-carboxamide [483];
3-(4-(4-methylpiperazin-1-yl)quinazolin-6-yl)-N-(1-methylpiperidin-4-yl)-1H-pyrrolo[2,3-b]pyridine-5-carboxamide [484];
N-(1-methylpiperidin-4-yl)-3-(4-((1-methylpiperidin-4-yl)oxy)quinazolin-6-yl)-1H-pyrrolo[2,3-b]pyridine-5-carboxamide [485];
N-(1-methylpiperidin-4-yl)-3-(4-((1-methylpiperidin-4-yl)amino)quinazolin-6-yl)-1H-pyrrolo[2,3-b]pyridine-5-carboxamide [486];
(3-(2-methyl-2H-indazol-5-yl)-1H-pyrrolo[2,3-b]pyridin-5-yl)(4-methylpiperazin-1-yl)methanone [487];
(4-methylpiperazin-1-yl)(3-(thieno[2,3-c]pyridin-2-yl)-1H-pyrrolo[2,3-b]pyridin-5-yl)methanone [488];
7-(5-(4-methylpiperazine-1-carbonyl)-1H-pyrrolo[2,3-b]pyridin-3-yl)-3,4-dihydropyrrolo[1,2-a]pyrazin-1(2H)-one [489];
(3-(imidazo[1,2-a]pyridin-6-yl)-1H-pyrrolo[2,3-b]pyridin-5-yl)(4-methylpiperazin-1-yl)methanone [490];
N-(2,2-difluoroethyl)-6-(5-(4-methylpiperazine-1-carbonyl)-1H-pyrrolo[2,3-b]pyridin-3-yl)imidazo[1,2-a]pyridine-3-carboxamide [491];
(3-(imidazo[1,2-a]pyrimidin-6-yl)-1H-pyrrolo[2,3-b]pyridin-5-yl)(4-methylpiperazin-1-yl)methanone [492];
(3-(4-fluoro-1-isopropyl-2-methyl-1H-benzo[d]imidazol-6-yl)-1H-pyrrolo[2,3-b]pyridin-5-yl)(4-methylpiperazin-1-yl)methanone [493];
(3-(4-fluoro-2-methyl-1-(1-methylpiperidin-4-yl)-1H-benzo[d]imidazol-6-yl)-1H-pyrrolo[2,3-b]pyridin-5-yl)(4-methylpiperazin-1-yl)methanone [494];
(3-(1-isopropyl-2-methyl-1H-imidazo[4,5-b]pyridin-6-yl)-1H-pyrrolo[2,3-b]pyridin-5-yl)(4-methylpiperazin-1-yl)methanone [495];
(3-(1-isopropyl-2-methyl-1H-imidazo[4,5-c]pyridin-6-yl)-1H-pyrrolo[2,3-b]pyridin-5-yl)(4-methylpiperazin-1-yl)methanone [496];
(3-([1,2,4]triazolo[1,5-a]pyridin-6-yl)-1H-pyrrolo[2,3-b]pyridin-5-yl)(4-methylpiperazin-1-yl)methanone [497];
(3-([1,2,4]triazolo[1,5-a]pyridin-7-yl)-1H-pyrrolo[2,3-b]pyridin-5-yl)(4-methylpiperazin-1-yl)methanone [498];
(3-(1-methyl-1H-benzo[d][1,2,3]triazol-6-yl)-1H-pyrrolo[2,3-b]pyridin-5-yl)(4-methylpiperazin-1-yl)methanone [499];
(3-(imidazo[1,2-b]pyridazin-6-yl)-1H-pyrrolo[2,3-b]pyridin-5-yl)(4-methylpiperazin-1-yl)methanone [500];
(4-methylpiperazin-1-yl)(3-(pyrazolo[1,5-a]pyridin-5-yl)-1H-pyrrolo[2,3-b]pyridin-5-yl)methanone [501];
(3-(3-(1-methyl-1H-pyrazol-4-yl)pyrazolo[1,5-a]pyridin-5-yl)-1H-pyrrolo[2,3-b]pyridin-5-yl)(4-methylpiperazin-1-yl)methanone [502];
(4-methylpiperazin-1-yl)(3-(3-(pyridin-3-yl)pyrazolo[1,5-a]pyridin-5-yl)-1H-pyrrolo[2,3-b]pyridin-5-yl)methanone [503];
N-(2,2-difluoroethyl)-5-(5-(4-methylpiperazine-1-carbonyl)-1H-pyrrolo[2,3-b]pyridin-3-yl)pyrazolo[1,5-a]pyridine-3-carboxamide [504];
(R)-5-(5-(4-methylpiperazine-1-carbonyl)-1H-pyrrolo[2,3-b]pyridin-3-yl)-N-(1,1,1-trifluoropropan-2-yl)pyrazolo[1,5-a]pyridine-3-carboxamide [505];
5-(5-(4-methylpiperazine-1-carbonyl)-1H-pyrrolo[2,3-b]pyridin-3-yl)-N-(1-methylpiperidin-4-yl)pyrazolo[1,5-a]pyridine-3-carboxamide [506];
5-(5-(4-methylpiperazine-1-carbonyl)-1H-pyrrolo[2,3-b]pyridin-3-yl)-N-(pyridin-3-yl)pyrazolo[1,5-a]pyridine-3-carboxamide [507];
(4-methylpiperazin-1-yl)(3-(3-(piperidine-1-carbonyl)pyrazolo[1,5-a]pyridin-5-yl)-1H-pyrrolo[2,3-b]pyridin-5-yl)methanone [508];
(4-methylpiperazin-1-yl)(5-(5-(4-methylpiperazine-1-carbonyl)-1H-pyrrolo[2,3-b]pyridin-3-yl)pyrazolo[1,5-a]pyridin-3-yl)methanone [509];
(4-methylpiperazin-1-yl)(3-(3-(morpholine-4-carbonyl)pyrazolo[1,5-a]pyridin-5-yl)-1H-pyrrolo[2,3-b]pyridin-5-yl)methanone [510];
6-(5-(4-methylpiperazine-1-carbonyl)-1H-pyrrolo[2,3-b]pyridin-3-yl)spiro[indene-1,4′-piperidin]-3(2H)-one [512];
1′-methyl-6-(5-(4-methylpiperazine-1-carbonyl)-1H-pyrrolo[2,3-b]pyridin-3-yl)spiro[indene-1,4′-piperidin]-3(2H)-one [513];
5-(5-(4-methylpiperazine-1-carbonyl)-1H-pyrrolo[2,3-b]pyridin-3-yl)isoindolin-1-one [514];
3,3-dimethyl-5-(5-(4-methylpiperazine-1-carbonyl)-1H-pyrrolo[2,3-b]pyridin-3-yl)isoindolin-1-one [515];
6′-(5-(4-methylpiperazine-1-carbonyl)-1H-pyrrolo[2,3-b]pyridin-3-yl)spiro[cyclohexane-1,1′-isoindolin]-3′-one [516];
(3-(chroman-6-yl)-1H-pyrrolo[2,3-b]pyridin-5-yl)(4-methylpiperazin-1-yl)methanone [517];
6-(5-(4-methylpiperazine-1-carbonyl)-1H-pyrrolo[2,3-b]pyridin-3-yl)-3,4-dihydroisoquinolin-1(2H)-one [518];
4,4-dimethyl-6-(5-(4-methylpiperazine-1-carbonyl)-1H-pyrrolo[2,3-b]pyridin-3-yl)-3,4-dihydroisoquinolin-1(2H)-one [519];
7-(5-(4-methylpiperazine-1-carbonyl)-1H-pyrrolo[2,3-b]pyridin-3-yl)spiro[chromane-2,4′-piperidin]-4-one [520];
8-(5-(4-methylpiperazine-1-carbonyl)-1H-pyrrolo[2,3-b]pyridin-3-yl)-3,4-dihydrobenzo[f][1,4]oxazepin-5(2H)-one [521];
(4-methylpiperazin-1-yl)(3-(quinolin-6-yl)-1H-pyrrolo[2,3-b]pyridin-5-yl)methanone [522];
(4-methylpiperazin-1-yl)(3-(quinoxalin-6-yl)-1H-pyrrolo[2,3-b]pyridin-5-yl)methanone [523];
(4-methylpiperazin-1-yl)(3-(quinazolin-6-yl)-1H-pyrrolo[2,3-b]pyridin-5-yl)methanone [524];
(3-(4-methoxyquinazolin-6-yl)-1H-pyrrolo[2,3-b]pyridin-5-yl)(4-methylpiperazin-1-yl)methanone [525];
(4-methylpiperazin-1-yl)(3-(4-(4-methylpiperazin-1-yl)quinazolin-6-yl)-1H-pyrrolo[2,3-b]pyridin-5-yl)methanone [526];
(4-methylpiperazin-1-yl)(3-(4-((1-methylpiperidin-4-yl)oxy)quinazolin-6-yl)-1H-pyrrolo[2,3-b]pyridin-5-yl)methanone [527];
(4-methylpiperazin-1-yl)(3-(4-((1-methylpiperidin-4-yl)amino)quinazolin-6-yl)-1H-pyrrolo[2,3-b]pyridin-5-yl)methanone [528];
(3-(2-methyl-2H-indazol-5-yl)-1H-pyrrolo[2,3-b]pyridin-5-yl)(3-methyl-3,8-diazabicyclo[3.2.1]octan-8-yl)methanone [529];
(3-methyl-3,8-diazabicyclo[3.2.1]octan-8-yl)(3-(thieno[2,3-c]pyridin-2-yl)-1H-pyrrolo[2,3-b]pyridin-5-yl)methanone [530];
7-(5-(3-methyl-3,8-diazabicyclo[3.2.1]octane-8-carbonyl)-1H-pyrrolo[2,3-b]pyridin-3-yl)-3,4-dihydropyrrolo[1,2-a]pyrazin-1(2H)-one [531];
(3-(imidazo[1,2-a]pyridin-6-yl)-1H-pyrrolo[2,3-b]pyridin-5-yl)(3-methyl-3,8-diazabicyclo[3.2.1]octan-8-yl)methanone [532];
N-(2,2-difluoroethyl)-6-(5-(3-methyl-3,8-diazabicyclo[3.2.1]octane-8-carbonyl)-1H-pyrrolo[2,3-b]pyridin-3-yl)imidazo[1,2-a]pyridine-3-carboxamide [533];
(3-(imidazo[1,2-a]pyrimidin-6-yl)-1H-pyrrolo[2,3-b]pyridin-5-yl)(3-methyl-3,8-diazabicyclo[3.2.1]octan-8-yl)methanone [534];
(3-(4-fluoro-1-isopropyl-2-methyl-1H-benzo[d]imidazol-6-yl)-1H-pyrrolo[2,3-b]pyridin-5-yl)(3-methyl-3,8-diazabicyclo[3.2.1]octan-8-yl)methanone [535];
(3-(4-fluoro-2-methyl-1-(1-methylpiperidin-4-yl)-1H-benzo[d]imidazol-6-yl)-1H-pyrrolo[2,3-b]pyridin-5-yl)(3-methyl-3,8-diazabicyclo[3.2.1]octan-8-yl)methanone [536];
(3-(1-isopropyl-2-methyl-1H-imidazo[4,5-b]pyridin-6-yl)-1H-pyrrolo[2,3-b]pyridin-5-yl)(3-methyl-3,8-diazabicyclo[3.2.1]octan-8-yl)methanone [537];
(3-(1-isopropyl-2-methyl-1H-imidazo[4,5-c]pyridin-6-yl)-1H-pyrrolo[2,3-b]pyridin-5-yl)(3-methyl-3,8-diazabicyclo[3.2.1]octan-8-yl)methanone [538];
(3-([1,2,4]triazolo[1,5-a]pyridin-6-yl)-1H-pyrrolo[2,3-b]pyridin-5-yl)(3-methyl-3,8-diazabicyclo[3.2.1]octan-8-yl)methanone [539];
(3-([1,2,4]triazolo[1,5-a]pyridin-7-yl)-1H-pyrrolo[2,3-b]pyridin-5-yl)(3-methyl-3,8-diazabicyclo[3.2.1]octan-8-yl)methanone [540];
(3-(1-methyl-1H-benzo[d][1,2,3]triazol-6-yl)-1H-pyrrolo[2,3-b]pyridin-5-yl)(3-methyl-3,8-diazabicyclo[3.2.1]octan-8-yl)methanone [541];
(3-(imidazo[1,2-b]pyridazin-6-yl)-1H-pyrrolo[2,3-b]pyridin-5-yl)(3-methyl-3,8-diazabicyclo[3.2.1]octan-8-yl)methanone [542];
(3-methyl-3,8-diazabicyclo[3.2.1]octan-8-yl)(3-(pyrazolo[1,5-a]pyridin-5-yl)-1H-pyrrolo[2,3-b]pyridin-5-yl)methanone [543];
(3-(3-(1-methyl-1H-pyrazol-4-yl)pyrazolo[1,5-a]pyridin-5-yl)-1H-pyrrolo[2,3-b]pyridin-5-yl)(3-methyl-3,8-diazabicyclo[3.2.1]octan-8-yl)methanone [544];
(3-methyl-3,8-diazabicyclo[3.2.1]octan-8-yl)(3-(3-(pyridin-3-yl)pyrazolo[1,5-a]pyridin-5-yl)-1H-pyrrolo[2,3-b]pyridin-5-yl)methanone [545];
N-(2,2-difluoroethyl)-5-(5-(3-methyl-3,8-diazabicyclo[3.2.1]octane-8-carbonyl)-1H-pyrrolo[2,3-b]pyridin-3-yl)pyrazolo[1,5-a]pyridine-3-carboxamide [546];
5-(5-(3-methyl-3,8-diazabicyclo[3.2.1]octane-8-carbonyl)-1H-pyrrolo[2,3-b]pyridin-3-yl)-N-((R)-1,1,1-trifluoropropan-2-yl)pyrazolo[1,5-a]pyridine-3-carboxamide [547];
5-(5-(3-methyl-3,8-diazabicyclo[3.2.1]octane-8-carbonyl)-1H-pyrrolo[2,3-b]pyridin-3-yl)-N-(1-methylpiperidin-4-yl)pyrazolo[1,5-a]pyridine-3-carboxamide [548];
5-(5-(3-methyl-3,8-diazabicyclo[3.2.1]octane-8-carbonyl)-1H-pyrrolo[2,3-b]pyridin-3-yl)-N-(pyridin-3-yl)pyrazolo[1,5-a]pyridine-3-carboxamide [549];
(5-(5-(3-methyl-3,8-diazabicyclo[3.2.1]octane-8-carbonyl)-1H-pyrrolo[2,3-b]pyridin-3-yl)pyrazolo[1,5-a]pyridin-3-yl)(piperidin-1-yl)methanone [550];
(5-(5-(3-methyl-3,8-diazabicyclo[3.2.1]octane-8-carbonyl)-1H-pyrrolo[2,3-b]pyridin-3-yl)pyrazolo[1,5-a]pyridin-3-yl)(4-methylpiperazin-1-yl)methanone [551];
(5-(5-(3-methyl-3,8-diazabicyclo[3.2.1]octane-8-carbonyl)-1H-pyrrolo[2,3-b]pyridin-3-yl)pyrazolo[1,5-a]pyridin-3-yl)(morpholino)methanone [552];
6-(5-(3-methyl-3,8-diazabicyclo[3.2.1]octane-8-carbonyl)-1H-pyrrolo[2,3-b]pyridin-3-yl)spiro[indene-1,4′-piperidin]-3(2H)-one [554];
1′-methyl-6-(5-(3-methyl-3,8-diazabicyclo[3.2.1]octane-8-carbonyl)-1H-pyrrolo[2,3-b]pyridin-3-yl)spiro[indene-1,4′-piperidin]-3(2H)-one [555];
5-(5-(3-methyl-3,8-diazabicyclo[3.2.1]octane-8-carbonyl)-1H-pyrrolo[2,3-b]pyridin-3-yl)isoindolin-1-one [556];
3,3-dimethyl-5-(5-(3-methyl-3,8-diazabicyclo[3.2.1]octane-8-carbonyl)-1H-pyrrolo[2,3-b]pyridin-3-yl)isoindolin-1-one [557];
6′-(5-(3-methyl-3,8-diazabicyclo[3.2.1]octane-8-carbonyl)-1H-pyrrolo[2,3-b]pyridin-3-yl)spiro[cyclohexane-1,1′-isoindolin]-3′-one [558];
(3-(chroman-6-yl)-1H-pyrrolo[2,3-b]pyridin-5-yl)(3-methyl-3,8-diazabicyclo[3.2.1]octan-8-yl)methanone [559];
6-(5-(3-methyl-3,8-diazabicyclo[3.2.1]octane-8-carbonyl)-1H-pyrrolo[2,3-b]pyridin-3-yl)-3,4-dihydroisoquinolin-1(2H)-one [560];
4,4-dimethyl-6-(5-(3-methyl-3,8-diazabicyclo[3.2.1]octane-8-carbonyl)-1H-pyrrolo[2,3-b]pyridin-3-yl)-3,4-dihydroisoquinolin-1(2H)-one [561];
7-(5-(3-methyl-3,8-diazabicyclo[3.2.1]octane-8-carbonyl)-1H-pyrrolo[2,3-b]pyridin-3-yl)spiro[chromane-2,4′-piperidin]-4-one [562];
8-(5-(3-methyl-3,8-diazabicyclo[3.2.1]octane-8-carbonyl)-1H-pyrrolo[2,3-b]pyridin-3-yl)-3,4-dihydrobenzo[f][1,4]oxazepin-5(2H)-one [563];
(3-methyl-3,8-diazabicyclo[3.2.1]octan-8-yl)(3-(quinolin-6-yl)-1H-pyrrolo[2,3-b]pyridin-5-yl)methanone [564];
(3-methyl-3,8-diazabicyclo[3.2.1]octan-8-yl)(3-(quinoxalin-6-yl)-1H-pyrrolo[2,3-b]pyridin-5-yl)methanone [565];
(3-methyl-3,8-diazabicyclo[3.2.1]octan-8-yl)(3-(quinazolin-6-yl)-1H-pyrrolo[2,3-b]pyridin-5-yl)methanone [566];
(3-(4-methoxyquinazolin-6-yl)-1H-pyrrolo[2,3-b]pyridin-5-yl)(3-methyl-3,8-diazabicyclo[3.2.1]octan-8-yl)methanone [567];
(3-methyl-3,8-diazabicyclo[3.2.1]octan-8-yl)(3-(4-(4-methylpiperazin-1-yl)quinazolin-6-yl)-1H-pyrrolo[2,3-b]pyridin-5-yl)methanone [568];
(3-methyl-3,8-diazabicyclo[3.2.1]octan-8-yl)(3-(4-((1-methylpiperidin-4-yl)oxy)quinazolin-6-yl)-1H-pyrrolo[2,3-b]pyridin-5-yl)methanone [569];
(3-methyl-3,8-diazabicyclo[3.2.1]octan-8-yl)(3-(4-((1-methylpiperidin-4-yl)amino)quinazolin-6-yl)-1H-pyrrolo[2,3-b]pyridin-5-yl)methanone [570];
(3-(2-methyl-2H-indazol-5-yl)-1H-pyrrolo[2,3-b]pyridin-5-yl)(2-methyl-5,6-dihydroimidazo[1,2-a]pyrazin-7(8H)-yl)methanone [571];
(2-methyl-5,6-dihydroimidazo[1,2-a]pyrazin-7(8H)-yl)(3-(thieno[2,3-c]pyridin-2-yl)-1H-pyrrolo[2,3-b]pyridin-5-yl)methanone [572];
7-(5-(2-methyl-5,6,7,8-tetrahydroimidazo[1,2-a]pyrazine-7-carbonyl)-1H-pyrrolo[2,3-b]pyridin-3-yl)-3,4-dihydropyrrolo[1,2-a]pyrazin-1(2H)-one [573];
(3-(imidazo[1,2-a]pyridin-6-yl)-1H-pyrrolo[2,3-b]pyridin-5-yl)(2-methyl-5,6-dihydroimidazo[1,2-a]pyrazin-7(8H)-yl)methanone [574];
(3-(3-(((2,2-difluoroethyl)amino)methyl)imidazo[1,2-a]pyridin-6-yl)-1H-pyrrolo[2,3-b]pyridin-5-yl)(2-methyl-5,6-dihydroimidazo[1,2-a]pyrazin-7(8H)-yl)methanone [575];
(3-(imidazo[1,2-a]pyrimidin-6-yl)-1H-pyrrolo[2,3-b]pyridin-5-yl)(2-methyl-5,6-dihydroimidazo[1,2-a]pyrazin-7(8H)-yl)methanone [576];
(3-(4-fluoro-1-isopropyl-2-methyl-1H-benzo[d]imidazol-6-yl)-1H-pyrrolo[2,3-b]pyridin-5-yl)(2-methyl-5,6-dihydroimidazo[1,2-a]pyrazin-7(8H)-yl)methanone [577];
(3-(4-fluoro-2-methyl-1-(1-methylpiperidin-4-yl)-1H-benzo[d]imidazol-6-yl)-1H-pyrrolo[2,3-b]pyridin-5-yl)(2-methyl-5,6-dihydroimidazo[1,2-a]pyrazin-7(8H)-yl)methanone [578];
(3-(1-isopropyl-2-methyl-1H-imidazo[4,5-b]pyridin-6-yl)-1H-pyrrolo[2,3-b]pyridin-5-yl)(2-methyl-5,6-dihydroimidazo[1,2-a]pyrazin-7(8H)-yl)methanone [579];
(3-(1-isopropyl-2-methyl-1H-imidazo[4,5-c]pyridin-6-yl)-1H-pyrrolo[2,3-b]pyridin-5-yl)(2-methyl-5,6-dihydroimidazo[1,2-a]pyrazin-7(8H)-yl)methanone [580];
(3-([1,2,4]triazolo[1,5-a]pyridin-6-yl)-1H-pyrrolo[2,3-b]pyridin-5-yl)(2-methyl-5,6-dihydroimidazo[1,2-a]pyrazin-7(8H)-yl)methanone [581];
(3-([1,2,4]triazolo[1,5-a]pyridin-7-yl)-1H-pyrrolo[2,3-b]pyridin-5-yl)(2-methyl-5,6-dihydroimidazo[1,2-a]pyrazin-7(8H)-yl)methanone [582];
(3-(1-methyl-1H-benzo[d][1,2,3]triazol-6-yl)-1H-pyrrolo[2,3-b]pyridin-5-yl)(2-methyl-5,6-dihydroimidazo[1,2-a]pyrazin-7(8H)-yl)methanone [583];
(3-(imidazo[1,2-b]pyridazin-6-yl)-1H-pyrrolo[2,3-b]pyridin-5-yl)(2-methyl-5,6-dihydroimidazo[1,2-a]pyrazin-7(8H)-yl)methanone [584];
(2-methyl-5,6-dihydroimidazo[1,2-a]pyrazin-7(8H)-yl)(3-(pyrazolo[1,5-a]pyridin-5-yl)-1H-pyrrolo[2,3-b]pyridin-5-yl)methanone [585];
(3-(3-(1-methyl-1H-pyrazol-4-yl)pyrazolo[1,5-a]pyridin-5-yl)-1H-pyrrolo[2,3-b]pyridin-5-yl)(2-methyl-5,6-dihydroimidazo[1,2-a]pyrazin-7(8H)-yl)methanone [586];
(2-methyl-5,6-dihydroimidazo[1,2-a]pyrazin-7(8H)-yl)(3-(3-(pyridin-3-yl)pyrazolo[1,5-a]pyridin-5-yl)-1H-pyrrolo[2,3-b]pyridin-5-yl)methanone [587];
N-(2,2-difluoroethyl)-5-(5-(2-methyl-5,6,7,8-tetrahydroimidazo[1,2-a]pyrazine-7-carbonyl)-1H-pyrrolo[2,3-b]pyridin-3-yl)pyrazolo[1,5-a]pyridine-3-carboxamide [588];
(R)-5-(5-(2-methyl-5,6,7,8-tetrahydroimidazo[1,2-a]pyrazine-7-carbonyl)-1H-pyrrolo[2,3-b]pyridin-3-yl)-N-(1,1,1-trifluoropropan-2-yl)pyrazolo[1,5-a]pyridine-3-carboxamide [589];
5-(5-(2-methyl-5,6,7,8-tetrahydroimidazo[1,2-a]pyrazine-7-carbonyl)-1H-pyrrolo[2,3-b]pyridin-3-yl)-N-(1-methylpiperidin-4-yl)pyrazolo[1,5-a]pyridine-3-carboxamide [590];
5-(5-(2-methyl-5,6,7,8-tetrahydroimidazo[1,2-a]pyrazine-7-carbonyl)-1H-pyrrolo[2,3-b]pyridin-3-yl)-N-(pyridin-3-yl)pyrazolo[1,5-a]pyridine-3-carboxamide [591];
(5-(5-(2-methyl-5,6,7,8-tetrahydroimidazo[1,2-a]pyrazine-7-carbonyl)-1H-pyrrolo[2,3-b]pyridin-3-yl)pyrazolo[1,5-a]pyridin-3-yl)(piperidin-1-yl)methanone [592];
(5-(5-(2-methyl-5,6,7,8-tetrahydroimidazo[1,2-a]pyrazine-7-carbonyl)-1H-pyrrolo[2,3-b]pyridin-3-yl)pyrazolo[1,5-a]pyridin-3-yl)(4-methylpiperazin-1-yl)methanone [593];
(5-(5-(2-methyl-5,6,7,8-tetrahydroimidazo[1,2-a]pyrazine-7-carbonyl)-1H-pyrrolo[2,3-b]pyridin-3-yl)pyrazolo[1,5-a]pyridin-3-yl)(morpholino)methanone [594];
6-(5-(2-methyl-5,6,7,8-tetrahydroimidazo[1,2-a]pyrazine-7-carbonyl)-1H-pyrrolo[2,3-b]pyridin-3-yl)spiro[indene-1,4′-piperidin]-3(2H)-one [596];
1′-methyl-6-(5-(2-methyl-5,6,7,8-tetrahydroimidazo[1,2-a]pyrazine-7-carbonyl)-1H-pyrrolo[2,3-b]pyridin-3-yl)spiro[indene-1,4′-piperidin]-3(2H)-one [597];
5-(5-(2-methyl-5,6,7,8-tetrahydroimidazo[1,2-a]pyrazine-7-carbonyl)-1H-pyrrolo[2,3-b]pyridin-3-yl)isoindolin-1-one [598];
3,3-dimethyl-5-(5-(2-methyl-5,6,7,8-tetrahydroimidazo[1,2-a]pyrazine-7-carbonyl)-1H-pyrrolo[2,3-b]pyridin-3-yl)isoindolin-1-one [599];
6′-(5-(2-methyl-5,6,7,8-tetrahydroimidazo[1,2-a]pyrazine-7-carbonyl)-1H-pyrrolo[2,3-b]pyridin-3-yl)spiro[cyclohexane-1,1′-isoindolin]-3′-one [600];
(3-(chroman-6-yl)-1H-pyrrolo[2,3-b]pyridin-5-yl)(2-methyl-5,6-dihydroimidazo[1,2-a]pyrazin-7(8H)-yl)methanone [601];
6-(5-(2-methyl-5,6,7,8-tetrahydroimidazo[1,2-a]pyrazine-7-carbonyl)-1H-pyrrolo[2,3-b]pyridin-3-yl)-3,4-dihydroisoquinolin-1(2H)-one [602];
4,4-dimethyl-6-(5-(2-methyl-5,6,7,8-tetrahydroimidazo[1,2-a]pyrazine-7-carbonyl)-1H-pyrrolo[2,3-b]pyridin-3-yl)-3,4-dihydroisoquinolin-1(2H)-one [603];
7-(5-(2-methyl-5,6,7,8-tetrahydroimidazo[1,2-a]pyrazine-7-carbonyl)-1H-pyrrolo[2,3-b]pyridin-3-yl)spiro[chromane-2,4′-piperidin]-4-one [604];
8-(5-(2-methyl-5,6,7,8-tetrahydroimidazo[1,2-a]pyrazine-7-carbonyl)-1H-pyrrolo[2,3-b]pyridin-3-yl)-3,4-dihydrobenzo[f][1,4]oxazepin-5(2H)-one [605];
(2-methyl-5,6-dihydroimidazo[1,2-a]pyrazin-7(8H)-yl)(3-(quinolin-6-yl)-1H-pyrrolo[2,3-b]pyridin-5-yl)methanone [606];
(2-methyl-5,6-dihydroimidazo[1,2-a]pyrazin-7(8H)-yl)(3-(quinoxalin-6-yl)-1H-pyrrolo[2,3-b]pyridin-5-yl)methanone [607];
(2-methyl-5,6-dihydroimidazo[1,2-a]pyrazin-7(8H)-yl)(3-(quinazolin-6-yl)-1H-pyrrolo[2,3-b]pyridin-5-yl)methanone [608];
(3-(4-methoxyquinazolin-6-yl)-1H-pyrrolo[2,3-b]pyridin-5-yl)(2-methyl-5,6-dihydroimidazo[1,2-a]pyrazin-7(8H)-yl)methanone [609];
(2-methyl-5,6-dihydroimidazo[1,2-a]pyrazin-7(8H)-yl)(3-(4-(4-methylpiperazin-1-yl)quinazolin-6-yl)-1H-pyrrolo[2,3-b]pyridin-5-yl)methanone [610];
(2-methyl-5,6-dihydroimidazo[1,2-a]pyrazin-7(8H)-yl)(3-(4-((1-methylpiperidin-4-yl)oxy)quinazolin-6-yl)-1H-pyrrolo[2,3-b]pyridin-5-yl)methanone [611];
(2-methyl-5,6-dihydroimidazo[1,2-a]pyrazin-7(8H)-yl)(3-(4-((1-methylpiperidin-4-yl)amino)quinazolin-6-yl)-1H-pyrrolo[2,3-b]pyridin-5-yl)methanone [612];
1-methyl-N-(3-(2-methyl-2H-indazol-5-yl)-1H-pyrrolo[2,3-b]pyridin-5-yl)piperidine-4-carboxamide [613];
1-methyl-N-(3-(thieno[2,3-c]pyridin-2-yl)-1H-pyrrolo[2,3-b]pyridin-5-yl)piperidine-4-carboxamide [614];
1-methyl-N-(3-(1-oxo-1,2,3,4-tetrahydropyrrolo[1,2-a]pyrazin-7-yl)-1H-pyrrolo[2,3-b]pyridin-5-yl)piperidine-4-carboxamide [615];
N-(3-(imidazo[1,2-a]pyridin-6-yl)-1H-pyrrolo[2,3-b]pyridin-5-yl)-1-methylpiperidine-4-carboxamide [616];
N-(2,2-difluoroethyl)-6-(5-(1-methylpiperidine-4-carboxamido)-1H-pyrrolo[2,3-b]pyridin-3-yl)imidazo[1,2-a]pyridine-3-carboxamide [617];
N-(3-(imidazo[1,2-a]pyrimidin-6-yl)-1H-pyrrolo[2,3-b]pyridin-5-yl)-1-methylpiperidine-4-carboxamide [618];
N-(3-(4-fluoro-1-isopropyl-2-methyl-1H-benzo[d]imidazol-6-yl)-1H-pyrrolo[2,3-b]pyridin-5-yl)-1-methylpiperidine-4-carboxamide [619];
N-(3-(4-fluoro-2-methyl-1-(1-methylpiperidin-4-yl)-1H-benzo[d]imidazol-6-yl)-1H-pyrrolo[2,3-b]pyridin-5-yl)-1-methylpiperidine-4-carboxamide [620];
N-(3-(1-isopropyl-2-methyl-1H-imidazo[4,5-b]pyridin-6-yl)-1H-pyrrolo[2,3-b]pyridin-5-yl)-1-methylpiperidine-4-carboxamide [621];
N-(3-(1-isopropyl-2-methyl-1H-imidazo[4,5-c]pyridin-6-yl)-1H-pyrrolo[2,3-b]pyridin-5-yl)-1-methylpiperidine-4-carboxamide [622];
N-(3-([1,2,4]triazolo[1,5-a]pyridin-6-yl)-1H-pyrrolo[2,3-b]pyridin-5-yl)-1-methylpiperidine-4-carboxamide [623];
N-(3-([1,2,4]triazolo[1,5-a]pyridin-7-yl)-1H-pyrrolo[2,3-b]pyridin-5-yl)-1-methylpiperidine-4-carboxamide [624];
1-methyl-N-(3-(1-methyl-1H-benzo[d][1,2,3]triazol-6-yl)-1H-pyrrolo[2,3-b]pyridin-5-yl)piperidine-4-carboxamide [625];
N-(3-(imidazo[1,2-b]pyridazin-6-yl)-1H-pyrrolo[2,3-b]pyridin-5-yl)-1-methylpiperidine-4-carboxamide [626];
1-methyl-N-(3-(pyrazolo[1,5-a]pyridin-5-yl)-1H-pyrrolo[2,3-b]pyridin-5-yl)piperidine-4-carboxamide [627];
1-methyl-N-(3-(3-(1-methyl-1H-pyrazol-4-yl)pyrazolo[1,5-a]pyridin-5-yl)-1H-pyrrolo[2,3-b]pyridin-5-yl)piperidine-4-carboxamide [628];
1-methyl-N-(3-(3-(pyridin-3-yl)pyrazolo[1,5-a]pyridin-5-yl)-1H-pyrrolo[2,3-b]pyridin-5-yl)piperidine-4-carboxamide [629];
N-(2,2-difluoroethyl)-5-(5-(1-methylpiperidine-4-carboxamido)-1H-pyrrolo[2,3-b]pyridin-3-yl)pyrazolo[1,5-a]pyridine-3-carboxamide [630];
(R)-5-(5-(1-methylpiperidine-4-carboxamido)-1H-pyrrolo[2,3-b]pyridin-3-yl)-N-(1,1,1-trifluoropropan-2-yl)pyrazolo[1,5-a]pyridine-3-carboxamide [631];
N-(1-methylpiperidin-4-yl)-5-(5-(1-methylpiperidine-4-carboxamido)-1H-pyrrolo[2,3-b]pyridin-3-yl)pyrazolo[1,5-a]pyridine-3-carboxamide [632];
5-(5-(1-methylpiperidine-4-carboxamido)-1H-pyrrolo[2,3-b]pyridin-3-yl)-N-(pyridin-3-yl)pyrazolo[1,5-a]pyridine-3-carboxamide [633];
1-methyl-N-(3-(3-(piperidine-1-carbonyl)pyrazolo[1,5-a]pyridin-5-yl)-1H-pyrrolo[2,3-b]pyridin-5-yl)piperidine-4-carboxamide [634];
1-methyl-N-(3-(3-(4-methylpiperazine-1-carbonyl)pyrazolo[1,5-a]pyridin-5-yl)-1H-pyrrolo[2,3-b]pyridin-5-yl)piperidine-4-carboxamide [635];
1-methyl-N-(3-(3-(morpholine-4-carbonyl)pyrazolo[1,5-a]pyridin-5-yl)-1H-pyrrolo[2,3-b]pyridin-5-yl)piperidine-4-carboxamide [636];
1-methyl-N-(3-(3-oxo-2,3-dihydrospiro[indene-1,4′-piperidin]-6-yl)-1H-pyrrolo[2,3-b]pyridin-5-yl)piperidine-4-carboxamide [638];
1-methyl-N-(3-(1′-methyl-3-oxo-2,3-dihydrospiro[indene-1,4′-piperidin]-6-yl)-1H-pyrrolo[2,3-b]pyridin-5-yl)piperidine-4-carboxamide [639];
1-methyl-N-(3-(1-oxoisoindolin-5-yl)-1H-pyrrolo[2,3-b]pyridin-5-yl)piperidine-4-carboxamide [640];
N-(3-(3,3-dimethyl-1-oxoisoindolin-5-yl)-1H-pyrrolo[2,3-b]pyridin-5-yl)-1-methylpiperidine-4-carboxamide [641];
1-methyl-N-(3-(3′-oxospiro[cyclohexane-1,1′-isoindolin]-6′-yl)-1H-pyrrolo[2,3-b]pyridin-5-yl)piperidine-4-carboxamide [642];
N-(3-(chroman-6-yl)-1H-pyrrolo[2,3-b]pyridin-5-yl)-1-methylpiperidine-4-carboxamide [643];
1-methyl-N-(3-(1-oxo-1,2,3,4-tetrahydroisoquinolin-6-yl)-1H-pyrrolo[2,3-b]pyridin-5-yl)piperidine-4-carboxamide [644];
N-(3-(4,4-dimethyl-1-oxo-1,2,3,4-tetrahydroisoquinolin-6-yl)-1H-pyrrolo[2,3-b]pyridin-5-yl)-1-methylpiperidine-4-carboxamide [645];
1-methyl-N-(3-(4-oxospiro[chromane-2,4′-piperidin]-7-yl)-1H-pyrrolo[2,3-b]pyridin-5-yl)piperidine-4-carboxamide [646];
1-methyl-N-(3-(5-oxo-2,3,4,5-tetrahydrobenzo[f][1,4]oxazepin-8-yl)-1H-pyrrolo[2,3-b]pyridin-5-yl)piperidine-4-carboxamide [647];
1-methyl-N-(3-(quinolin-6-yl)-1H-pyrrolo[2,3-b]pyridin-5-yl)piperidine-4-carboxamide [648];
1-methyl-N-(3-(quinoxalin-6-yl)-1H-pyrrolo[2,3-b]pyridin-5-yl)piperidine-4-carboxamide [649];
1-methyl-N-(3-(quinoxalin-6-yl)-1H-pyrrolo[2,3-b]pyridin-5-yl)piperidine-4-carboxamide [650];
N-(3-(4-methoxyquinazolin-6-yl)-1H-pyrrolo[2,3-b]pyridin-5-yl)-1-methylpiperidine-4-carboxamide [651];
1-methyl-N-(3-(4-(4-methylpiperazin-1-yl)quinazolin-6-yl)-1H-pyrrolo[2,3-b]pyridin-5-yl)piperidine-4-carboxamide [652];
1-methyl-N-(3-(4-((1-methylpiperidin-4-yl)oxy)quinazolin-6-yl)-1H-pyrrolo[2,3-b]pyridin-5-yl)piperidine-4-carboxamide [653];
1-methyl-N-(3-(4-((1-methylpiperidin-4-yl)amino)quinazolin-6-yl)-1H-pyrrolo[2,3-b]pyridin-5-yl)piperidine-4-carboxamide [654];
3-(2-methyl-2H-indazol-5-yl)-N-(1-(piperidin-4-yl)-1H-pyrazol-4-yl)-1H-pyrrolo[2,3-b]pyridine-5-carboxamide [655];
N-(1-(piperidin-4-yl)-1H-pyrazol-4-yl)-3-(thieno[2,3-c]pyridin-2-yl)-1H-pyrrolo[2,3-b]pyridine-5-carboxamide [656];
3-(1-oxo-1,2,3,4-tetrahydropyrrolo[1,2-a]pyrazin-7-yl)-N-(1-(piperidin-4-yl)-1H-pyrazol-4-yl)-1H-pyrrolo[2,3-b]pyridine-5-carboxamide [657];
3-(imidazo[1,2-a]pyridin-6-yl)-N-(1-(piperidin-4-yl)-1H-pyrazol-4-yl)-1H-pyrrolo[2,3-b]pyridine-5-carboxamide [658];
N-(2,2-difluoroethyl)-6-(5-((1-(piperidin-4-yl)-1H-pyrazol-4-yl)carbamoyl)-1H-pyrrolo[2,3-b]pyridin-3-yl)imidazo[1,2-a]pyridine-3-carboxamide [659];
3-(imidazo[1,2-a]pyrimidin-6-yl)-N-(1-(piperidin-4-yl)-1H-pyrazol-4-yl)-1H-pyrrolo[2,3-b]pyridine-5-carboxamide [660];
3-(4-fluoro-1-isopropyl-2-methyl-1H-benzo[d]imidazol-6-yl)-N-(1-(piperidin-4-yl)-1H-pyrazol-4-yl)-1H-pyrrolo[2,3-b]pyridine-5-carboxamide [661];
3-(4-fluoro-2-methyl-1-(1-methylpiperidin-4-yl)-1H-benzo[d]imidazol-6-yl)-N-(1-(piperidin-4-yl)-1H-pyrazol-4-yl)-1H-pyrrolo[2,3-b]pyridine-5-carboxamide [662];
3-(1-isopropyl-2-methyl-1H-imidazo[4,5-b]pyridin-6-yl)-N-(1-(piperidin-4-yl)-1H-pyrazol-4-yl)-1H-pyrrolo[2,3-b]pyridine-5-carboxamide [663];
3-(1-isopropyl-2-methyl-1H-imidazo[4,5-c]pyridin-6-yl)-N-(1-(piperidin-4-yl)-1H-pyrazol-4-yl)-1H-pyrrolo[2,3-b]pyridine-5-carboxamide [664];
3-([1,2,4]triazolo[1,5-a]pyridin-6-yl)-N-(1-(piperidin-4-yl)-1H-pyrazol-4-yl)-1H-pyrrolo[2,3-b]pyridine-5-carboxamide [665];
3-([1,2,4]triazolo[1,5-a]pyridin-7-yl)-N-(1-(piperidin-4-yl)-1H-pyrazol-4-yl)-1H-pyrrolo[2,3-b]pyridine-5-carboxamide [666];
3-(1-methyl-1H-benzo[d][1,2,3]triazol-6-yl)-N-(1-(piperidin-4-yl)-1H-pyrazol-4-yl)-1H-pyrrolo[2,3-b]pyridine-5-carboxamide [667];
3-(imidazo[1,2-b]pyridazin-6-yl)-N-(1-(piperidin-4-yl)-1H-pyrazol-4-yl)-1H-pyrrolo[2,3-b]pyridine-5-carboxamide [668];
N-(1-(piperidin-4-yl)-1H-pyrazol-4-yl)-3-(pyrazolo[1,5-a]pyridin-5-yl)-1H-pyrrolo[2,3-b]pyridine-5-carboxamide [669];
3-(3-(1-methyl-1H-pyrazol-4-yl)pyrazolo[1,5-a]pyridin-5-yl)-N-(1-(piperidin-4-yl)-1H-pyrazol-4-yl)-1H-pyrrolo[2,3-b]pyridine-5-carboxamide [670];
N-(1-(piperidin-4-yl)-1H-pyrazol-4-yl)-3-(3-(pyridin-3-yl)pyrazolo[1,5-a]pyridin-5-yl)-1H-pyrrolo[2,3-b]pyridine-5-carboxamide [671];
3-(3-((2,2-difluoroethyl)carbamoyl)pyrazolo[1,5-a]pyridin-5-yl)-N-(1-(piperidin-4-yl)-1H-pyrazol-4-yl)-1H-pyrrolo[2,3-b]pyridine-5-carboxamide [672];
(R)-N-(1-(piperidin-4-yl)-1H-pyrazol-4-yl)-3-(3-((1,1,1-trifluoropropan-2-yl)carbamoyl)pyrazolo[1,5-a]pyridin-5-yl)-1H-pyrrolo[2,3-b]pyridine-5-carboxamide [673];
3-(3-((1-methylpiperidin-4-yl)carbamoyl)pyrazolo[1,5-a]pyridin-5-yl)-N-(1-(piperidin-4-yl)-1H-pyrazol-4-yl)-1H-pyrrolo[2,3-b]pyridine-5-carboxamide [674];
N-(1-(piperidin-4-yl)-1H-pyrazol-4-yl)-3-(3-(pyridin-3-ylcarbamoyl)pyrazolo[1,5-a]pyridin-5-yl)-1H-pyrrolo[2,3-b]pyridine-5-carboxamide [675];
N-(1-(piperidin-4-yl)-1H-pyrazol-4-yl)-3-(3-(piperidine-1-carbonyl)pyrazolo[1,5-a]pyridin-5-yl)-1H-pyrrolo[2,3-b]pyridine-5-carboxamide [676];
3-(3-(4-methylpiperazine-1-carbonyl)pyrazolo[1,5-a]pyridin-5-yl)-N-(1-(piperidin-4-yl)-1H-pyrazol-4-yl)-1H-pyrrolo[2,3-b]pyridine-5-carboxamide [677];
3-(3-(morpholine-4-carbonyl)pyrazolo[1,5-a]pyridin-5-yl)-N-(1-(piperidin-4-yl)-1H-pyrazol-4-yl)-1H-pyrrolo[2,3-b]pyridine-5-carboxamide [678];
3-(3-oxo-2,3-dihydrospiro[indene-1,4′-piperidin]-6-yl)-N-(1-(piperidin-4-yl)-1H-pyrazol-4-yl)-1H-pyrrolo[2,3-b]pyridine-5-carboxamide [680];
3-(1′-methyl-3-oxo-2,3-dihydrospiro[indene-1,4′-piperidin]-6-yl)-N-(1-(piperidin-4-yl)-1H-pyrazol-4-yl)-1H-pyrrolo[2,3-b]pyridine-5-carboxamide [681];
3-(1-oxoisoindolin-5-yl)-N-(1-(piperidin-4-yl)-1H-pyrazol-4-yl)-1H-pyrrolo[2,3-b]pyridine-5-carboxamide [682];
3-(3,3-dimethyl-1-oxoisoindolin-5-yl)-N-(1-(piperidin-4-yl)-1H-pyrazol-4-yl)-1H-pyrrolo[2,3-b]pyridine-5-carboxamide [683];
3-(3′-oxospiro[cyclohexane-1,1′-isoindolin]-6′-yl)-N-(1-(piperidin-4-yl)-1H-pyrazol-4-yl)-1H-pyrrolo[2,3-b]pyridine-5-carboxamide [684];
3-(chroman-6-yl)-N-(1-(piperidin-4-yl)-1H-pyrazol-4-yl)-1H-pyrrolo[2,3-b]pyridine-5-carboxamide [685];
3-(1-oxo-1,2,3,4-tetrahydroisoquinolin-6-yl)-N-(1-(piperidin-4-yl)-1H-pyrazol-4-yl)-1H-pyrrolo[2,3-b]pyridine-5-carboxamide [686];
3-(4,4-dimethyl-1-oxo-1,2,3,4-tetrahydroisoquinolin-6-yl)-N-(1-(piperidin-4-yl)-1H-pyrazol-4-yl)-1H-pyrrolo[2,3-b]pyridine-5-carboxamide [687];
3-(4-oxospiro[chromane-2,4′-piperidin]-7-yl)-N-(1-(piperidin-4-yl)-1H-pyrazol-4-yl)-1H-pyrrolo[2,3-b]pyridine-5-carboxamide [688];
3-(5-oxo-2,3,4,5-tetrahydrobenzo[1,4]oxazepin-8-yl)-N-(1-(piperidin-4-yl)-1H-pyrazol-4-yl)-1H-pyrrolo[2,3-b]pyridine-5-carboxamide [689];
N-(1-(piperidin-4-yl)-1H-pyrazol-4-yl)-3-(quinolin-6-yl)-1H-pyrrolo[2,3-b]pyridine-5-carboxamide [690];
N-(1-(piperidin-4-yl)-1H-pyrazol-4-yl)-3-(quinoxalin-6-yl)-1H-pyrrolo[2,3-b]pyridine-5-carboxamide [691];
N-(1-(piperidin-4-yl)-1H-pyrazol-4-yl)-3-(quinazolin-6-yl)-1H-pyrrolo[2,3-b]pyridine-5-carboxamide [692];
3-(4-methoxyquinazolin-6-yl)-N-(1-(piperidin-4-yl)-1H-pyrazol-4-yl)-1H-pyrrolo[2,3-b]pyridine-5-carboxamide [693];
3-(4-(4-methylpiperazin-1-yl)quinazolin-6-yl)-N-(1-(piperidin-4-yl)-1H-pyrazol-4-yl)-1H-pyrrolo[2,3-b]pyridine-5-carboxamide [694];
3-(4-((1-methylpiperidin-4-yl)oxy)quinazolin-6-yl)-N-(1-(piperidin-4-yl)-1H-pyrazol-4-yl)-1H-pyrrolo[2,3-b]pyridine-5-carboxamide [695];
3-(4-((1-methylpiperidin-4-yl)amino)quinazolin-6-yl)-N-(1-(piperidin-4-yl)-1H-pyrazol-4-yl)-1H-pyrrolo[2,3-b]pyridine-5-carboxamide [696];
3-(2-methyl-2H-indazol-5-yl)-N-(1-(1-methylpiperidin-4-yl)-1H-pyrazol-4-yl)-1H-pyrrolo[2,3-b]pyridine-5-carboxamide [697];
N-(1-(1-methylpiperidin-4-yl)-1H-pyrazol-4-yl)-3-(thieno[2,3-c]pyridin-2-yl)-1H-pyrrolo[2,3-b]pyridine-5-carboxamide [698];
N-(1-(1-methylpiperidin-4-yl)-1H-pyrazol-4-yl)-3-(1-oxo-1,2,3,4-tetrahydropyrrolo[1,2-a]pyrazin-7-yl)-1H-pyrrolo[2,3-b]pyridine-5-carboxamide [699];
3-(imidazo[1,2-a]pyridin-6-yl)-N-(1-(1-methylpiperidin-4-yl)-1H-pyrazol-4-yl)-1H-pyrrolo[2,3-b]pyridine-5-carboxamide [700];
N-(2,2-difluoroethyl)-6-(5-((1-(1-methylpiperidin-4-yl)-1H-pyrazol-4-yl)carbamoyl)-1H-pyrrolo[2,3-b]pyridin-3-yl)imidazo[1,2-a]pyridine-3-carboxamide [701];
3-(imidazo[1,2-a]pyrimidin-6-yl)-N-(1-(1-methylpiperidin-4-yl)-1H-pyrazol-4-yl)-1H-pyrrolo[2,3-b]pyridine-5-carboxamide [702];
3-(4-fluoro-1-isopropyl-2-methyl-1H-benzo[d]imidazol-6-yl)-N-(1-(1-methylpiperidin-4-yl)-1H-pyrazol-4-yl)-1H-pyrrolo[2,3-b]pyridine-5-carboxamide [703];
3-(4-fluoro-2-methyl-1-(1-methylpiperidin-4-yl)-1H-benzo[d]imidazol-6-yl)-N-(1-(1-methylpiperidin-4-yl)-1H-pyrazol-4-yl)-1H-pyrrolo[2,3-b]pyridine-5-carboxamide [704];
3-(1-isopropyl-2-methyl-1H-imidazo[4,5-b]pyridin-6-yl)-N-(1-(1-methylpiperidin-4-yl)-1H-pyrazol-4-yl)-1H-pyrrolo[2,3-b]pyridine-5-carboxamide [705];
3-(1-isopropyl-2-methyl-1H-imidazo[4,5-c]pyridin-6-yl)-N-(1-(1-methylpiperidin-4-yl)-1H-pyrazol-4-yl)-1H-pyrrolo[2,3-b]pyridine-5-carboxamide [706];
3-([1,2,4]triazolo[1,5-a]pyridin-6-yl)-N-(1-(1-methylpiperidin-4-yl)-1H-pyrazol-4-yl)-1H-pyrrolo[2,3-b]pyridine-5-carboxamide [707];
3-([1,2,4]triazolo[1,5-a]pyridin-7-yl)-N-(1-(1-methylpiperidin-4-yl)-1H-pyrazol-4-yl)-1H-pyrrolo[2,3-b]pyridine-5-carboxamide [708];
3-(1-methyl-1H-benzo[d][1,2,3]triazol-6-yl)-N-(1-(1-methylpiperidin-4-yl)-1H-pyrazol-4-yl)-1H-pyrrolo[2,3-b]pyridine-5-carboxamide [709];
3-(imidazo[1,2-b]pyridazin-6-yl)-N-(1-(1-methylpiperidin-4-yl)-1H-pyrazol-4-yl)-1H-pyrrolo[2,3-b]pyridine-5-carboxamide [710];
N-(1-(1-methylpiperidin-4-yl)-1H-pyrazol-4-yl)-3-(pyrazolo[1,5-a]pyridin-5-yl)-1H-pyrrolo[2,3-b]pyridine-5-carboxamide [711];
3-(3-(1-methyl-1H-pyrazol-4-yl)pyrazolo[1,5-a]pyridin-5-yl)-N-(1-(1-methylpiperidin-4-yl)-1H-pyrazol-4-yl)-1H-pyrrolo[2,3-b]pyridine-5-carboxamide [712];
N-(1-(1-methylpiperidin-4-yl)-1H-pyrazol-4-yl)-3-(3-(pyridin-3-yl)pyrazolo[1,5-a]pyridin-5-yl)-1H-pyrrolo[2,3-b]pyridine-5-carboxamide [713];
3-(3-((2,2-difluoroethyl)carbamoyl)pyrazolo[1,5-a]pyridin-5-yl)-N-(1-(1-methylpiperidin-4-yl)-1H-pyrazol-4-yl)-1H-pyrrolo[2,3-b]pyridine-5-carboxamide [714];
(R)-N-(1-(1-methylpiperidin-4-yl)-1H-pyrazol-4-yl)-3-(3-((1,1,1-trifluoropropan-2-yl)carbamoyl)pyrazolo[1,5-a]pyridin-5-yl)-1H-pyrrolo[2,3-b]pyridine-5-carboxamide [715];
N-(1-(1-methylpiperidin-4-yl)-1H-pyrazol-4-yl)-3-(3-((1-methylpiperidin-4-yl)carbamoyl)pyrazolo[1,5-a]pyridin-5-yl)-1H-pyrrolo[2,3-b]pyridine-5-carboxamide [716];
N-(1-(1-methylpiperidin-4-yl)-1H-pyrazol-4-yl)-3-(3-(pyridin-3-ylcarbamoyl)pyrazolo[1,5-a]pyridin-5-yl)-1H-pyrrolo[2,3-b]pyridine-5-carboxamide [717];
N-(1-(1-methylpiperidin-4-yl)-1H-pyrazol-4-yl)-3-(3-(piperidine-1-carbonyl)pyrazolo[1,5-a]pyridin-5-yl)-1H-pyrrolo[2,3-b]pyridine-5-carboxamide [718];
3-(3-(4-methylpiperazine-1-carbonyl)pyrazolo[1,5-a]pyridin-5-yl)-N-(1-(1-methylpiperidin-4-yl)-1H-pyrazol-4-yl)-1H-pyrrolo[2,3-b]pyridine-5-carboxamide [719];
N-(1-(1-methylpiperidin-4-yl)-1H-pyrazol-4-yl)-3-(3-(morpholine-4-carbonyl)pyrazolo[1,5-a]pyridin-5-yl)-1H-pyrrolo[2,3-b]pyridine-5-carboxamide [720];
N-(1-(1-methylpiperidin-4-yl)-1H-pyrazol-4-yl)-3-(3-oxo-2,3-dihydrospiro[indene-1,4′-piperidin]-6-yl)-1H-pyrrolo[2,3-b]pyridine-5-carboxamide [722];
3-(1′-methyl-3-oxo-2,3-dihydrospiro[indene-1,4′-piperidin]-6-yl)-N-(1-(1-methylpiperidin-4-yl)-1H-pyrazol-4-yl)-1H-pyrrolo[2,3-b]pyridine-5-carboxamide [723];
N-(1-(1-methylpiperidin-4-yl)-1H-pyrazol-4-yl)-3-(1-oxoisoindolin-5-yl)-1H-pyrrolo[2,3-b]pyridine-5-carboxamide [724];
3-(3,3-dimethyl-1-oxoisoindolin-5-yl)-N-(1-(1-methylpiperidin-4-yl)-1H-pyrazol-4-yl)-1H-pyrrolo[2,3-b]pyridine-5-carboxamide [725];
N-(1-(1-methylpiperidin-4-yl)-1H-pyrazol-4-yl)-3-(3′-oxospiro[cyclohexane-1,1′-isoindolin]-6′-yl)-1H-pyrrolo[2,3-b]pyridine-5-carboxamide [726];
3-(chroman-6-yl)-N-(1-(1-methylpiperidin-4-yl)-1H-pyrazol-4-yl)-1H-pyrrolo[2,3-b]pyridine-5-carboxamide [727];
N-(1-(1-methylpiperidin-4-yl)-1H-pyrazol-4-yl)-3-(1-oxo-1,2,3,4-tetrahydroisoquinolin-6-yl)-1H-pyrrolo[2,3-b]pyridine-5-carboxamide [728];
3-(4,4-dimethyl-1-oxo-1,2,3,4-tetrahydroisoquinolin-6-yl)-N-(1-(1-methylpiperidin-4-yl)-1H-pyrazol-4-yl)-1H-pyrrolo[2,3-b]pyridine-5-carboxamide [729];
N-(1-(1-methylpiperidin-4-yl)-1H-pyrazol-4-yl)-3-(4-oxospiro[chromane-2,4′-piperidin]-7-yl)-1H-pyrrolo[2,3-b]pyridine-5-carboxamide [730];
N-(1-(1-methylpiperidin-4-yl)-1H-pyrazol-4-yl)-3-(5-oxo-2,3,4,5-tetrahydrobenzo[f][1,4]oxazepin-8-yl)-1H-pyrrolo[2,3-b]pyridine-5-carboxamide [731];
N-(1-(1-methylpiperidin-4-yl)-1H-pyrazol-4-yl)-3-(quinolin-6-yl)-1H-pyrrolo[2,3-b]pyridine-5-carboxamide [732];
N-(1-(1-methylpiperidin-4-yl)-1H-pyrazol-4-yl)-3-(quinoxalin-6-yl)-1H-pyrrolo[2,3-b]pyridine-5-carboxamide [733];
N-(1-(1-methylpiperidin-4-yl)-1H-pyrazol-4-yl)-3-(quinazolin-6-yl)-1H-pyrrolo[2,3-b]pyridine-5-carboxamide [734];
3-(4-methoxyquinazolin-6-yl)-N-(1-(1-methylpiperidin-4-yl)-1H-pyrazol-4-yl)-1H-pyrrolo[2,3-b]pyridine-5-carboxamide [735];
3-(4-(4-methylpiperazin-1-yl)quinazolin-6-yl)-N-(1-(1-methylpiperidin-4-yl)-1H-pyrazol-4-yl)-1H-pyrrolo[2,3-b]pyridine-5-carboxamide [736];
N-(1-(1-methylpiperidin-4-yl)-1H-pyrazol-4-yl)-3-(4-((1-methylpiperidin-4-yl)oxy)quinazolin-6-yl)-1H-pyrrolo[2,3-b]pyridine-5-carboxamide [737];
N-(1-(1-methylpiperidin-4-yl)-1H-pyrazol-4-yl)-3-(4-((1-methylpiperidin-4-yl)amino)quinazolin-6-yl)-1H-pyrrolo[2,3-b]pyridine-5-carboxamide [738];
N-(5-fluoropyridin-3-yl)-3-(2-methyl-2H-indazol-5-yl)-1H-pyrrolo[2,3-b]pyridine-5-carboxamide [739];
N-(5-fluoropyridin-3-yl)-3-(thieno[2,3-c]pyridin-2-yl)-1H-pyrrolo[2,3-b]pyridine-5-carboxamide [740];
N-(5-fluoropyridin-3-yl)-3-(1-oxo-1,2,3,4-tetrahydropyrrolo[1,2-a]pyrazin-7-yl)-1H-pyrrolo[2,3-b]pyridine-5-carboxamide [741];
N-(5-fluoropyridin-3-yl)-3-(imidazo[1,2-a]pyridin-6-yl)-1H-pyrrolo[2,3-b]pyridine-5-carboxamide [742];
N-(2,2-difluoroethyl)-6-(5-((5-fluoropyridin-3-yl)carbamoyl)-1H-pyrrolo[2,3-b]pyridin-3-yl)imidazo[1,2-a]pyridine-3-carboxamide [743];
N-(5-fluoropyridin-3-yl)-3-(imidazo[1,2-a]pyrimidin-6-yl)-1H-pyrrolo[2,3-b]pyridine-5-carboxamide [744];
3-(4-fluoro-1-isopropyl-2-methyl-1H-benzo[d]imidazol-6-yl)-N-(5-fluoropyridin-3-yl)-1H-pyrrolo[2,3-b]pyridine-5-carboxamide [745];
3-(4-fluoro-2-methyl-1-(1-methylpiperidin-4-yl)-1H-benzo[d]imidazol-6-yl)-N-(5-fluoropyridin-3-yl)-1H-pyrrolo[2,3-b]pyridine-5-carboxamide [746];
N-(5-fluoropyridin-3-yl)-3-(1-isopropyl-2-methyl-1H-imidazo[4,5-b]pyridin-6-yl)-1H-pyrrolo[2,3-b]pyridine-5-carboxamide [747];
N-(5-fluoropyridin-3-yl)-3-(1-isopropyl-2-methyl-1H-imidazo[4,5-c]pyridin-6-yl)-1H-pyrrolo[2,3-b]pyridine-5-carboxamide [748];
3-([1,2,4]triazolo[1,5-a]pyridin-6-yl)-N-(5-fluoropyridin-3-yl)-1H-pyrrolo[2,3-b]pyridine-5-carboxamide [749];
3-([1,2,4]triazolo[1,5-a]pyridin-7-yl)-N-(5-fluoropyridin-3-yl)-1H-pyrrolo[2,3-b]pyridine-5-carboxamide [750];
N-(5-fluoropyridin-3-yl)-3-(1-methyl-1H-benzo[d][1,2,3]triazol-6-yl)-1H-pyrrolo[2,3-b]pyridine-5-carboxamide [751];
N-(5-fluoropyridin-3-yl)-3-(imidazo[1,2-b]pyridazin-6-yl)-1H-pyrrolo[2,3-b]pyridine-5-carboxamide [752];
N-(5-fluoropyridin-3-yl)-3-(pyrazolo[1,5-a]pyridin-5-yl)-1H-pyrrolo[2,3-b]pyridine-5-carboxamide [753];
N-(5-fluoropyridin-3-yl)-3-(3-(1-methyl-1H-pyrazol-4-yl)pyrazolo[1,5-a]pyridin-5-yl)-1H-pyrrolo[2,3-b]pyridine-5-carboxamide [754];
N-(5-fluoropyridin-3-yl)-3-(3-(pyridin-3-yl)pyrazolo[1,5-a]pyridin-5-yl)-1H-pyrrolo[2,3-b]pyridine-5-carboxamide [755];
3-(3-((2,2-difluoroethyl)carbamoyl)pyrazolo[1,5-a]pyridin-5-yl)-N-(5-fluoropyridin-3-yl)-1H-pyrrolo[2,3-b]pyridine-5-carboxamide [756];
(R)-N-(5-fluoropyridin-3-yl)-3-(3-((1,1,1-trifluoropropan-2-yl)carbamoyl)pyrazolo[1,5-a]pyridin-5-yl)-1H-pyrrolo[2,3-b]pyridine-5-carboxamide [757];
N-(5-fluoropyridin-3-yl)-3-(3-((1-methylpiperidin-4-yl)carbamoyl)pyrazolo[1,5-a]pyridin-5-yl)-1H-pyrrolo[2,3-b]pyridine-5-carboxamide [758];
N-(5-fluoropyridin-3-yl)-3-(3-(pyridin-3-ylcarbamoyl)pyrazolo[1,5-a]pyridin-5-yl)-1H-pyrrolo[2,3-b]pyridine-5-carboxamide [759];
N-(5-fluoropyridin-3-yl)-3-(3-(piperidine-1-carbonyl)pyrazolo[1,5-a]pyridin-5-yl)-1H-pyrrolo[2,3-b]pyridine-5-carboxamide [760];
N-(5-fluoropyridin-3-yl)-3-(3-(4-methylpiperazine-1-carbonyl)pyrazolo[1,5-a]pyridin-5-yl)-1H-pyrrolo[2,3-b]pyridine-5-carboxamide [761];
N-(5-fluoropyridin-3-yl)-3-(3-(morpholine-4-carbonyl)pyrazolo[1,5-a]pyridin-5-yl)-1H-pyrrolo[2,3-b]pyridine-5-carboxamide [762];
N-(5-fluoropyridin-3-yl)-3-(3-oxo-2,3-dihydrospiro[indene-1,4′-piperidin]-6-yl)-1H-pyrrolo[2,3-b]pyridine-5-carboxamide [764];
N-(5-fluoropyridin-3-yl)-3-(1′-methyl-3-oxo-2,3-dihydrospiro[indene-1,4′-piperidin]-6-yl)-1H-pyrrolo[2,3-b]pyridine-5-carboxamide [765];
N-(5-fluoropyridin-3-yl)-3-(1-oxoisoindolin-5-yl)-1H-pyrrolo[2,3-b]pyridine-5-carboxamide [766];
3-(3,3-dimethyl-1-oxoisoindolin-5-yl)-N-(5-fluoropyridin-3-yl)-1H-pyrrolo[2,3-b]pyridine-5-carboxamide [767];
N-(5-fluoropyridin-3-yl)-3-(3′-oxospiro[cyclohexane-1,1′-isoindolin]-6′-yl)-1H-pyrrolo[2,3-b]pyridine-5-carboxamide [768];
3-(chroman-6-yl)-N-(5-fluoropyridin-3-yl)-1H-pyrrolo[2,3-b]pyridine-5-carboxamide [769];
N-(5-fluoropyridin-3-yl)-3-(1-oxo-1,2,3,4-tetrahydroisoquinolin-6-yl)-1H-pyrrolo[2,3-b]pyridine-5-carboxamide [770];
3-(4,4-dimethyl-1-oxo-1,2,3,4-tetrahydroisoquinolin-6-yl)-N-(5-fluoropyridin-3-yl)-1H-pyrrolo[2,3-b]pyridine-5-carboxamide [771];
N-(5-fluoropyridin-3-yl)-3-(4-oxospiro[chromane-2,4′-piperidin]-7-yl)-1H-pyrrolo[2,3-b]pyridine-5-carboxamide [772];
N-(5-fluoropyridin-3-yl)-3-(5-oxo-2,3,4,5-tetrahydrobenzo[f][1,4]oxazepin-8-yl)-1H-pyrrolo[2,3-b]pyridine-5-carboxamide [773];
N-(5-fluoropyridin-3-yl)-3-(quinolin-6-yl)-1H-pyrrolo[2,3-b]pyridine-5-carboxamide [774];
N-(5-fluoropyridin-3-yl)-3-(quinoxalin-6-yl)-1H-pyrrolo[2,3-b]pyridine-5-carboxamide [775];
N-(5-fluoropyridin-3-yl)-3-(quinazolin-6-yl)-1H-pyrrolo[2,3-b]pyridine-5-carboxamide [776];
N-(5-fluoropyridin-3-yl)-3-(4-methoxyquinazolin-6-yl)-1H-pyrrolo[2,3-b]pyridine-5-carboxamide [777];
N-(5-fluoropyridin-3-yl)-3-(4-(4-methylpiperazin-1-yl)quinazolin-6-yl)-1H-pyrrolo[2,3-b]pyridine-5-carboxamide [778];
N-(5-fluoropyridin-3-yl)-3-(4-((1-methylpiperidin-4-yl)oxy)quinazolin-6-yl)-1H-pyrrolo[2,3-b]pyridine-5-carboxamide [779];
N-(5-fluoropyridin-3-yl)-3-(4-((1-methylpiperidin-4-yl)amino)quinazolin-6-yl)-1H-pyrrolo[2,3-b]pyridine-5-carboxamide [780];
3-(2-methyl-2H-indazol-5-yl)-N-(6-(4-methylpiperazin-1-yl)pyridin-3-yl)-1H-pyrrolo[2,3-b]pyridine-5-carboxamide [781];
N-(6-(4-methylpiperazin-1-yl)pyridin-3-yl)-3-(thieno[2,3-c]pyridin-2-yl)-1H-pyrrolo[2,3-b]pyridine-5-carboxamide [782];
N-(6-(4-methylpiperazin-1-yl)pyridin-3-yl)-3-(1-oxo-1,2,3,4-tetrahydropyrrolo[1,2-a]pyrazin-7-yl)-1H-pyrrolo[2,3-b]pyridine-5-carboxamide [783];
3-(imidazo[1,2-a]pyridin-6-yl)-N-(6-(4-methylpiperazin-1-yl)pyridin-3-yl)-1H-pyrrolo[2,3-b]pyridine-5-carboxamide [784];
N-(2,2-difluoroethyl)-6-(5-((6-(4-methylpiperazin-1-yl)pyridin-3-yl)carbamoyl)-1H-pyrrolo[2,3-b]pyridin-3-yl)imidazo[1,2-a]pyridine-3-carboxamide [785];
3-(imidazo[1,2-a]pyrimidin-6-yl)-N-(6-(4-methylpiperazin-1-yl)pyridin-3-yl)-1H-pyrrolo[2,3-b]pyridine-5-carboxamide [786];
3-(4-fluoro-1-isopropyl-2-methyl-1H-benzo[d]imidazol-6-yl)-N-(6-(4-methylpiperazin-1-yl)pyridin-3-yl)-1H-pyrrolo[2,3-b]pyridine-5-carboxamide [787];
3-(4-fluoro-2-methyl-1-(1-methylpiperidin-4-yl)-1H-benzo[d]imidazol-6-yl)-N-(6-(4-methylpiperazin-1-yl)pyridin-3-yl)-1H-pyrrolo[2,3-b]pyridine-5-carboxamide [788];
3-(1-isopropyl-2-methyl-1H-imidazo[4,5-b]pyridin-6-yl)-N-(6-(4-methylpiperazin-1-yl)pyridin-3-yl)-1H-pyrrolo[2,3-b]pyridine-5-carboxamide [789];
3-(1-isopropyl-2-methyl-1H-imidazo[4,5-c]pyridin-6-yl)-N-(6-(4-methylpiperazin-1-yl)pyridin-3-yl)-1H-pyrrolo[2,3-b]pyridine-5-carboxamide [790];
3-([1,2,4]triazolo[1,5-a]pyridin-6-yl)-N-(6-(4-methylpiperazin-1-yl)pyridin-3-yl)-1H-pyrrolo[2,3-b]pyridine-5-carboxamide [791];
3-([1,2,4]triazolo[1,5-a]pyridin-7-yl)-N-(6-(4-methylpiperazin-1-yl)pyridin-3-yl)-1H-pyrrolo[2,3-b]pyridine-5-carboxamide [792];
3-(1-methyl-1H-benzo[d][1,2,3]triazol-6-yl)-N-(6-(4-methylpiperazin-1-yl)pyridin-3-yl)-1H-pyrrolo[2,3-b]pyridine-5-carboxamide [793];
3-(imidazo[1,2-b]pyridazin-6-yl)-N-(6-(4-methylpiperazin-1-yl)pyridin-3-yl)-1H-pyrrolo[2,3-b]pyridine-5-carboxamide [794];
N-(6-(4-methylpiperazin-1-yl)pyridin-3-yl)-3-(pyrazolo[1,5-a]pyridin-5-yl)-1H-pyrrolo[2,3-b]pyridine-5-carboxamide [795];
3-(3-(1-methyl-1H-pyrazol-4-yl)pyrazolo[1,5-a]pyridin-5-yl)-N-(6-(4-methylpiperazin-1-yl)pyridin-3-yl)-1H-pyrrolo[2,3-b]pyridine-5-carboxamide [796];
N-(6-(4-methylpiperazin-1-yl)pyridin-3-yl)-3-(3-(pyridin-3-yl)pyrazolo[1,5-a]pyridin-5-yl)-1H-pyrrolo[2,3-b]pyridine-5-carboxamide [797];
3-(3-((2,2-difluoroethyl)carbamoyl)pyrazolo[1,5-a]pyridin-5-yl)-N-(6-(4-methylpiperazin-1-yl)pyridin-3-yl)-1H-pyrrolo[2,3-b]pyridine-5-carboxamide [798];
(R)-N-(6-(4-methylpiperazin-1-yl)pyridin-3-yl)-3-(3-((1,1,1-trifluoropropan-2-yl)carbamoyl)pyrazolo[1,5-a]pyridin-5-yl)-1H-pyrrolo[2,3-b]pyridine-5-carboxamide [799];
N-(6-(4-methylpiperazin-1-yl)pyridin-3-yl)-3-(3-((1-methylpiperidin-4-yl)carbamoyl)pyrazolo[1,5-a]pyridin-5-yl)-1H-pyrrolo[2,3-b]pyridine-5-carboxamide [800];
N-(6-(4-methylpiperazin-1-yl)pyridin-3-yl)-3-(3-(pyridin-3-ylcarbamoyl)pyrazolo[1,5-a]pyridin-5-yl)-1H-pyrrolo[2,3-b]pyridine-5-carboxamide [801];
N-(6-(4-methylpiperazin-1-yl)pyridin-3-yl)-3-(3-(piperidine-1-carbonyl)pyrazolo[1,5-a]pyridin-5-yl)-1H-pyrrolo[2,3-b]pyridine-5-carboxamide [802];
N-(6-(4-methylpiperazin-1-yl)pyridin-3-yl)-3-(3-(4-methylpiperazine-1-carbonyl)pyrazolo[1,5-a]pyridin-5-yl)-1H-pyrrolo[2,3-b]pyridine-5-carboxamide [803];
N-(6-(4-methylpiperazin-1-yl)pyridin-3-yl)-3-(3-(morpholine-4-carbonyl)pyrazolo[1,5-a]pyridin-5-yl)-1H-pyrrolo[2,3-b]pyridine-5-carboxamide [804];
N-(6-(4-methylpiperazin-1-yl)pyridin-3-yl)-3-(3-oxo-2,3-dihydrospiro[indene-1,4′-piperidin]-6-yl)-1H-pyrrolo[2,3-b]pyridine-5-carboxamide [806];
3-(1′-methyl-3-oxo-2,3-dihydrospiro[indene-1,4′-piperidin]-6-yl)-N-(6-(4-methylpiperazin-1-yl)pyridin-3-yl)-1H-pyrrolo[2,3-b]pyridine-5-carboxamide [807];
N-(6-(4-methylpiperazin-1-yl)pyridin-3-yl)-3-(1-oxoisoindolin-5-yl)-1H-pyrrolo[2,3-b]pyridine-5-carboxamide [808];
3-(3,3-dimethyl-1-oxoisoindolin-5-yl)-N-(6-(4-methylpiperazin-1-yl)pyridin-3-yl)-1H-pyrrolo[2,3-b]pyridine-5-carboxamide [809];
N-(6-(4-methylpiperazin-1-yl)pyridin-3-yl)-3-(3′-oxospiro[cyclohexane-1,1′-isoindolin]-6′-yl)-1H-pyrrolo[2,3-b]pyridine-5-carboxamide [810];
3-(chroman-6-yl)-N-(6-(4-methylpiperazin-1-yl)pyridin-3-yl)-1H-pyrrolo[2,3-b]pyridine-5-carboxamide [811];
N-(6-(4-methylpiperazin-1-yl)pyridin-3-yl)-3-(1-oxo-1,2,3,4-tetrahydroisoquinolin-6-yl)-1H-pyrrolo[2,3-b]pyridine-5-carboxamide [812];
3-(4,4-dimethyl-1-oxo-1,2,3,4-tetrahydroisoquinolin-6-yl)-N-(6-(4-methylpiperazin-1-yl)pyridin-3-yl)-1H-pyrrolo[2,3-b]pyridine-5-carboxamide [813];
N-(6-(4-methylpiperazin-1-yl)pyridin-3-yl)-3-(4-oxospiro[chromane-2,4′-piperidin]-7-yl)-1H-pyrrolo[2,3-b]pyridine-5-carboxamide [814];
N-(6-(4-methylpiperazin-1-yl)pyridin-3-yl)-3-(5-oxo-2,3,4,5-tetrahydrobenzo[f][1,4]oxazepin-8-yl)-1H-pyrrolo[2,3-b]pyridine-5-carboxamide [815];
N-(6-(4-methylpiperazin-1-yl)pyridin-3-yl)-3-(quinolin-6-yl)-1H-pyrrolo[2,3-b]pyridine-5-carboxamide [816];
N-(6-(4-methylpiperazin-1-yl)pyridin-3-yl)-3-(quinoxalin-6-yl)-1H-pyrrolo[2,3-b]pyridine-5-carboxamide [817];
N-(6-(4-methylpiperazin-1-yl)pyridin-3-yl)-3-(quinazolin-6-yl)-1H-pyrrolo[2,3-b]pyridine-5-carboxamide [818];
3-(4-methoxyquinazolin-6-yl)-N-(6-(4-methylpiperazin-1-yl)pyridin-3-yl)-1H-pyrrolo[2,3-b]pyridine-5-carboxamide [819];
N-(6-(4-methylpiperazin-1-yl)pyridin-3-yl)-3-(4-(4-methylpiperazin-1-yl)quinazolin-6-yl)-1H-pyrrolo[2,3-b]pyridine-5-carboxamide [820];
N-(6-(4-methylpiperazin-1-yl)pyridin-3-yl)-3-(4-((1-methylpiperidin-4-yl)oxy)quinazolin-6-yl)-1H-pyrrolo[2,3-b]pyridine-5-carboxamide [821];
N-(6-(4-methylpiperazin-1-yl)pyridin-3-yl)-3-(4-((1-methylpiperidin-4-yl)amino)quinazolin-6-yl)-1H-pyrrolo[2,3-b]pyridine-5-carboxamide [822];
N-(3-(2-methyl-2H-indazol-5-yl)-1H-pyrrolo[2,3-b]pyridin-5-yl)-2-(piperazin-1-yl)isonicotinamide [823];
2-(piperazin-1-yl)-N-(3-(thieno[2,3-c]pyridin-2-yl)-1H-pyrrolo[2,3-b]pyridin-5-yl)isonicotinamide [824];
N-(3-(1-oxo-1,2,3,4-tetrahydropyrrolo[1,2-a]pyrazin-7-yl)-1H-pyrrolo[2,3-b]pyridin-5-yl)-2-(piperazin-1-yl)isonicotinamide [825];
N-(3-(imidazo[1,2-a]pyridin-6-yl)-1H-pyrrolo[2,3-b]pyridin-5-yl)-2-(piperazin-1-yl)isonicotinamide [826];
N-(2,2-difluoroethyl)-6-(5-(2-(piperazin-1-yl)isonicotinamido)-1H-pyrrolo[2,3-b]pyridin-3-yl)imidazo[1,2-a]pyridine-3-carboxamide [827];
N-(3-(imidazo[1,2-a]pyrimidin-6-yl)-1H-pyrrolo[2,3-b]pyridin-5-yl)-2-(piperazin-1-yl)isonicotinamide [828];
N-(3-(4-fluoro-1-isopropyl-2-methyl-1H-benzo[d]imidazol-6-yl)-1H-pyrrolo[2,3-b]pyridin-5-yl)-2-(piperazin-1-yl)isonicotinamide [829];
N-(3-(4-fluoro-2-methyl-1-(1-methylpiperidin-4-yl)-1H-benzo[d]imidazol-6-yl)-1H-pyrrolo[2,3-b]pyridin-5-yl)-2-(piperazin-1-yl)isonicotinamide [830];
N-(3-(1-isopropyl-2-methyl-1H-imidazo[4,5-1)]pyridin-6-yl)-1H-pyrrolo[2,3-b]pyridin-5-yl)-2-(piperazin-1-yl)isonicotinamide [831];
N-(3-(1-isopropyl-2-methyl-1H-imidazo[4,5-c]pyridin-6-yl)-1H-pyrrolo[2,3-b]pyridin-5-yl)-2-(piperazin-1-yl)isonicotinamide [832];
N-(3-([1,2,4]triazolo[1,5-a]pyridin-6-yl)-1H-pyrrolo[2,3-b]pyridin-5-yl)-2-(piperazin-1-yl)isonicotinamide [833];
N-(3-([1,2,4]triazolo[1,5-a]pyridin-7-yl)-1H-pyrrolo[2,3-b]pyridin-5-yl)-2-(piperazin-1-yl)isonicotinamide [834];
N-(3-(1-methyl-1H-benzo[d][1,2,3]triazol -6-yl)-1H-pyrrolo[2,3-b]pyridin-5-yl)-2-(piperazin-1-yl)isonicotinamide [835];
N-(3-(imidazo[1,2-b]pyridazin-6-yl)-1H-pyrrolo[2,3-b]pyridin-5-yl)-2-(piperazin-1-yl)isonicotinamide [836];
2-(piperazin-1-yl)-N-(3-(pyrazolo[1,5-a]pyridin-5-yl)-1H-pyrrolo[2,3-b]pyridin-5-yl)isonicotinamide [837];
N-(3-(3-(1-methyl-1H-pyrazol-4-yl)pyrazolo[1,5-a]pyridin-5-yl)-1H-pyrrolo[2,3-b]pyridin-5-yl)-2-(piperazin-1-yl)isonicotinamide [838];
2-(piperazin-1-yl)-N-(3-(3-(pyridin-3-yl)pyrazolo[1,5-a]pyridin-5-yl)-1H-pyrrolo[2,3-b]pyridin-5-yl)isonicotinamide [839];
N-(2,2-difluoroethyl)-5-(5-(2-(piperazin-1-yl)isonicotinamido)-1H-pyrrolo[2,3-b]pyridin-3-yl)pyrazolo[1,5-a]pyridine-3-carboxamide [840];
(R)-5-(5-(2-(piperazin-1-yl)isonicotinamido)-1H-pyrrolo[2,3-b]pyridin-3-yl)-N-(1,1,1-trifluoropropan-2-yl)pyrazolo[1,5-a]pyridine-3-carboxamide [841];
N-(1-methylpiperidin-4-yl)-5-(5-(2-(piperazin-1-yl)isonicotinamido)-1H-pyrrolo[2,3-b]pyridin-3-yl)pyrazolo[1,5-a]pyridine-3-carboxamide [842];
5-(5-(2-(piperazin-1-yl)isonicotinamido)-1H-pyrrolo[2,3-b]pyridin-3-yl)-N-(pyridin-3-yl)pyrazolo[1,5-a]pyridine-3-carboxamide [843];
2-(piperazin-1-yl)-N-(3-(3-(piperidine-1-carbonyl)pyrazolo[1,5-a]pyridin-5-yl)-1H-pyrrolo[2,3-b]pyridin-5-yl)isonicotinamide [844];
N-(3-(3-(4-methylpiperazine-1-carbonyl)pyrazolo[1,5-a]pyridin-5-yl)-1H-pyrrolo[2,3-b]pyridin-5-yl)-2-(piperazin-1-yl)isonicotinamide [845];
N-(3-(3-(morpholine-4-carbonyl)pyrazolo[1,5-a]pyridin-5-yl)-1H-pyrrolo[2,3-b]pyridin-5-yl)-2-(piperazin-1-yl)isonicotinamide [846];
N-(3-(3-oxo-2,3-dihydrospiro[indene-1,4′-piperidin]-6-yl)-1H-pyrrolo[2,3-b]pyridin-5-yl)-2-(piperazin-1-yl)isonicotinamide [848];
N-(3-(1′-methyl-3-oxo-2,3-dihydrospiro[indene-1,4′-piperidin]-6-yl)-1H-pyrrolo[2,3-b]pyridin-5-yl)-2-(piperazin-1-yl)isonicotinamide [849];
N-(3-(1-oxoisoindolin-5-yl)-1H-pyrrolo[2,3-b]pyridin-5-yl)-2-(piperazin-1-yl)isonicotinamide [850];
N-(3-(3,3-dimethyl-1-oxoisoindolin-5-yl)-1H-pyrrolo[2,3-b]pyridin-5-yl)-2-(piperazin-1-yl)isonicotinamide [851];
N-(3-(3′-oxospiro[cyclohexane-1,1′-isoindolin]-6′-yl)-1H-pyrrolo[2,3-b]pyridin-5-yl)-2-(piperazin-1-yl)isonicotinamide [852];
N-(3-(chroman-6-yl)-1H-pyrrolo[2,3-b]pyridin-5-yl)-2-(piperazin-1-yl)isonicotinamide [853];
N-(3-(1-oxo-1,2,3,4-tetrahydroisoquinolin-6-yl)-1H-pyrrolo[2,3-b]pyridin-5-yl)-2-(piperazin-1-yl)isonicotinamide [854];
N-(3-(4,4-dimethyl-1-oxo-1,2,3,4-tetrahydroisoquinolin-6-yl)-1H-pyrrolo[2,3-b]pyridin-5-yl)-2-(piperazin-1-yl)isonicotinamide [855];
N-(3-(4-oxospiro[chromane-2,4′-piperidin]-7-yl)-1H-pyrrolo[2,3-b]pyridin-5-yl)-2-(piperazin-1-yl)isonicotinamide [856];
N-(3-(5-oxo-2,3,4,5-tetrahydrobenzo[f][1,4]oxazepin-8-yl)-1H-pyrrolo[2,3-b]pyridin-5-yl)-2-(piperazin-1-yl)isonicotinamide [857];
2-(piperazin-1-yl)-N-(3-(quinolin-6-yl)-1H-pyrrolo[2,3-b]pyridin-5-yl)isonicotinamide [858];
2-(piperazin-1-yl)-N-(3-(quinoxalin-6-yl)-1H-pyrrolo[2,3-b]pyridin-5-yl)isonicotinamide [859];
2-(piperazin-1-yl)-N-(3-(quinazolin-6-yl)-1H-pyrrolo[2,3-b]pyridin-5-yl)isonicotinamide [860];
N-(3-(4-methoxyquinazolin-6-yl)-1H-pyrrolo[2,3-b]pyridin-5-yl)-2-(piperazin-1-yl)isonicotinamide [861];
N-(3-(4-(4-methylpiperazin-1-yl)quinazolin-6-yl)-1H-pyrrolo[2,3-b]pyridin-5-yl)-2-(piperazin-1-yl)isonicotinamide [862];
N-(3-(4-((1-methylpiperidin-4-yl)oxy)quinazolin-6-yl)-1H-pyrrolo[2,3-b]pyridin-5-yl)-2-(piperazin-1-yl)isonicotinamide [863];
N-(3-(4-((1-methylpiperidin-4-yl)amino)quinazolin-6-yl)-1H-pyrrolo[2,3-b]pyridin-5-yl)-2-(piperazin-1-yl)isonicotinamide [864];
3-(2-methyl-2H-indazol-5-yl)-N-(2-(4-methylpiperazin-1-yl)pyridin-4-yl)-1H-pyrrolo[2,3-b]pyridin-5-amine [865];
N-(2-(4-methylpiperazin-1-yl)pyridin-4-yl)-3-(thieno[2,3-c]pyridin-2-yl)-1H-pyrrolo[2,3-b]pyridin-5-amine [866];
7-(5-((2-(4-methylpiperazin-1-yl)pyridin-4-yl)amino)-1H-pyrrolo[2,3-b]pyridin-3-yl)-3,4-dihydropyrrolo[1,2-a]pyrazin-1(2H)-one [867];
3-(imidazo[1,2-a]pyridin-6-yl)-N-(2-(4-methylpiperazin-1-yl)pyridin-4-yl)-1H-pyrrolo[2,3-b]pyridin-5-amine [868];
N-(2,2-difluoroethyl)-6-(5-((2-(4-methylpiperazin-1-yl)pyridin-4-yl)amino)-1H-pyrrolo[2,3-b]pyridin-3-yl)imidazo[1,2-a]pyridine-3-carboxamide [869];
3-(imidazo[1,2-a]pyrimidin-6-yl)-N-(2-(4-methylpiperazin-1-yl)pyridin-4-yl)-1H-pyrrolo[2,3-b]pyridin-5-amine [870];
3-(4-fluoro-1-isopropyl-2-methyl-1H-benzo[d]imidazol-6-yl)-N-(2-(4-methylpiperazin-1-yl)pyridin-4-yl)-1H-pyrrolo[2,3-b]pyridin-5-amine [871];
3-(4-fluoro-2-methyl-1-(1-methylpiperidin-4-yl)-1H-benzo[d]imidazol-6-yl)-N-(2-(4-methylpiperazin-1-yl)pyridin-4-yl)-1H-pyrrolo[2,3-b]pyridin-5-amine [872];
3-(1-isopropyl-2-methyl-1H-imidazo[4,5-b]pyridin-6-yl)-N-(2-(4-methylpiperazin-1-yl)pyridin-4-yl)-1H-pyrrolo[2,3-b]pyridin-5-amine [873];
3-(1-isopropyl-2-methyl-1H-imidazo[4,5-c]pyridin-6-yl)-N-(2-(4-methylpiperazin-1-yl)pyridin-4-yl)-1H-pyrrolo[2,3-b]pyridin-5-amine [874];
3-([1,2,4]triazolo[1,5-a]pyridin-6-yl)-N-(2-(4-methylpiperazin-1-yl)pyridin-4-yl)-1H-pyrrolo[2,3-b]pyridin-5-amine [875];
3-([1,2,4]triazolo[1,5-a]pyridin-7-yl)-N-(2-(4-methylpiperazin-1-yl)pyridin-4-yl)-1H-pyrrolo[2,3-b]pyridin-5-amine [876];
3-(1-methyl-1H-benzo[d][1,2,3]triazol-6-yl)-N-(2-(4-methylpiperazin-1-yl)pyridin-4-yl)-1H-pyrrolo[2,3-b]pyridin-5-amine [877];
3-(imidazo[1,2-b]pyridazin-6-yl)-N-(2-(4-methylpiperazin-1-yl)pyridin-4-yl)-1H-pyrrolo[2,3-b]pyridin-5-amine [878];
N-(2-(4-methylpiperazin-1-yl)pyridin-4-yl)-3-(pyrazolo[1,5-a]pyridin-5-yl)-1H-pyrrolo[2,3-b]pyridin-5-amine [879];
3-(3-(1-methyl-1H-pyrazol-4-yl)pyrazolo[1,5-a]pyridin-5-yl)-N-(2-(4-methylpiperazin-1-yl)pyridin-4-yl)-1H-pyrrolo[2,3-b]pyridin-5-amine [880];
N-(2-(4-methylpiperazin-1-yl)pyridin-4-yl)-3-(3-(pyridin-3-yl)pyrazolo[1,5-a]pyridin-5-yl)-1H-pyrrolo[2,3-b]pyridin-5-amine [881];
N-(2,2-difluoroethyl)-5-(5-((2-(4-methylpiperazin-1-yl)pyridin-4-yl)amino)-1H-pyrrolo[2,3-b]pyridin-3-yl)pyrazolo[1,5-a]pyridine-3-carboxamide [882];
(R)-5-(5-((2-(4-methylpiperazin-1-yl)pyridin-4-yl)amino)-1H-pyrrolo[2,3-b]pyridin-3-yl)-N-(1,1,1-trifluoropropan-2-yl)pyrazolo[1,5-a]pyridine-3-carboxamide [883];
5-(5-((2-(4-methylpiperazin-1-yl)pyridin-4-yl)amino)-1H-pyrrolo[2,3-b]pyridin-3-yl)-N-(1-methylpiperidin-4-yl)pyrazolo[1,5-a]pyridine-3-carboxamide [884];
5-(5-((2-(4-methylpiperazin-1-yl)pyridin-4-yl)amino)-1H-pyrrolo[2,3-b]pyridin-3-yl)-N-(pyridin-3-yl)pyrazolo[1,5-a]pyridine-3-carboxamide [885];
(5-(5-((2-(4-methylpiperazin-1-yl)pyridin-4-yl)amino)-1H-pyrrolo[2,3-b]pyridin-3-yl)pyrazolo[1,5-a]pyridin-3-yl)(piperidin-1-yl)methanone [886];
(4-methylpiperazin-1-yl)(5-(5-((2-(4-methylpiperazin-1-yl)pyridin-4-yl)amino)-1H-pyrrolo[2,3-b]pyridin-3-yl)pyrazolo[1,5-a]pyridin-3-yl)methanone [887];
(5-(5-((2-(4-methylpiperazin-1-yl)pyridin-4-yl)amino)-1H-pyrrolo[2,3-b]pyridin-3-yl)pyrazolo[1,5-a]pyridin-3-yl)(morpholino)methanone [888];
6-(5-((2-(4-methylpiperazin-1-yl)pyridin-4-yl)amino)-1H-pyrrolo[2,3-b]pyridin-3-yl)spiro[indene-1,4′-piperidin]-3(2H)-one [890];
1′-methyl-6-(5-((2-(4-methylpiperazin-1-yl)pyridin-4-yl)amino)-1H-pyrrolo[2,3-b]pyridin-3-yl)spiro[indene-1,4′-piperidin]-3(2H)-one [891];
5-(5-((2-(4-methylpiperazin-1-yl)pyridin-4-yl)amino)-1H-pyrrolo[2,3-b]pyridin-3-yl)isoindolin-1-one [892];
3,3-dimethyl-5-(5-((2-(4-methylpiperazin-1-yl)pyridin-4-yl)amino)-1H-pyrrolo[2,3-b]pyridin-3-yl)isoindolin-1-one [893];
6′-(5-((2-(4-methylpiperazin-1-yl)pyridin-4-yl)amino)-1H-pyrrolo[2,3-b]pyridin-3-yl)spiro[cyclohexane-1,1′-isoindolin]-3′-one [894];
3-(chroman-6-yl)-N-(2-(4-methylpiperazin-1-yl)pyridin-4-yl)-1H-pyrrolo[2,3-b]pyridin-5-amine [895];
6-(5-((2-(4-methylpiperazin-1-yl)pyridin-4-yl)amino)-1H-pyrrolo[2,3-b]pyridin-3-yl)-3,4-dihydroisoquinolin-1(2H)-one [896];
4,4-dimethyl-6-(5-((2-(4-methylpiperazin-1-yl)pyridin-4-yl)amino)-1H-pyrrolo[2,3-b]pyridin-3-yl)-3,4-dihydroisoquinolin-1(2H)-one [897];
7-(5-((2-(4-methylpiperazin-1-yl)pyridin-4-yl)amino)-1H-pyrrolo[2,3-b]pyridin-3-yl)spiro[chromane-2,4′-piperidin]-4-one [898];
8-(5-((2-(4-methylpiperazin-1-yl)pyridin-4-yl)amino)-1H-pyrrolo[2,3-b]pyridin-3-yl)-3,4-dihydrobenzo[f][1,4]oxazepin-5(2H)-one [899];
N-(2-(4-methylpiperazin-1-yl)pyridin-4-yl)-3-(quinolin-6-yl)-1H-pyrrolo[2,3-b]pyridin-5-amine [900];
N-(2-(4-methylpiperazin-1-yl)pyridin-4-yl)-3-(quinoxalin-6-yl)-1H-pyrrolo[2,3-b]pyridin-5-amine [901];
N-(2-(4-methylpiperazin-1-yl)pyridin-4-yl)-3-(quinazolin-6-yl)-1H-pyrrolo[2,3-b]pyridin-5-amine [902];
3-(4-methoxyquinazolin-6-yl)-N-(2-(4-methylpiperazin-1-yl)pyridin-4-yl)-1H-pyrrolo[2,3-b]pyridin-5-amine [903];
N-(2-(4-methylpiperazin-1-yl)pyridin-4-yl)-3-(4-(4-methylpiperazin-1-yl)quinazolin-6-yl)-1H-pyrrolo[2,3-b]pyridin-5-amine [904];
N-(2-(4-methylpiperazin-1-yl)pyridin-4-yl)-3-(4-((1-methylpiperidin-4-yl)oxy)quinazolin-6-yl)-1H-pyrrolo[2,3-b]pyridin-5-amine [905];
6-(5-((2-(4-methylpiperazin-1-yl)pyridin-4-yl)amino)-1H-pyrrolo[2,3-b]pyridin-3-yl)-N-(1-methylpiperidin-4-yl)quinazolin-4-amine [906];
2-(5-phenyl-1H-pyrrolo[2,3-b]pyridin-3-yl)thieno[2,3-c]pyridine [908];
7-(5-phenyl-1H-pyrrolo[2,3-b]pyridin-3-yl)-3,4-dihydropyrrolo[1,2-a]pyrazin-1(2H)-one [909];
6-(5-phenyl-1H-pyrrolo[2,3-b]pyridin-3-yl)imidazo[1,2-a]pyridine [910];
N-(2,2-difluoroethyl)-6-(5-phenyl-1H-pyrrolo[2,3-b]pyridin-3-yl)imidazo[1,2-a]pyridine-3-carboxamide [911];
6-(5-phenyl-1H-pyrrolo[2,3-b]pyridin-3-yl)imidazo[1,2-a]pyrimidine [912];
4-fluoro-1-isopropyl-2-methyl-6-(5-phenyl-1H-pyrrolo[2,3-b]pyridin-3-yl)-1H-benzo[d]imidazole [913];
4-fluoro-2-methyl-1-(1-methylpiperidin-4-yl)-6-(5-phenyl-1H-pyrrolo[2,3-b]pyridin-3-yl)-1H-benzo[d]imidazole [914];
1-isopropyl-2-methyl-6-(5-phenyl-1H-pyrrolo[2,3-b]pyridin-3-yl)-1H-imidazo[4,5-b]pyridine [915];
1-isopropyl-2-methyl-6-(5-phenyl-1H-pyrrolo[2,3-b]pyridin-3-yl)-1H-imidazo[4,5-c]pyridine [916];
6-(5-phenyl-1H-pyrrolo[2,3-b]pyridin-3-yl)-[1,2,4]triazolo[1,5-a]pyridine [917];
7-(5-phenyl-1H-pyrrolo[2,3-b]pyridin-3-yl)-[1,2,4]triazolo[1,5-a]pyridine [918];
1-methyl-6-(5-phenyl-1H-pyrrolo[2,3-b]pyridin-3-yl)-1H-benzo[d][1,2,3]triazole [919];
6-(5-phenyl-1H-pyrrolo[2,3-b]pyridin-3-yl)imidazo[1,2-b]pyridazine [920];
5-phenyl-3-(pyrazolo[1,5-a]pyridin-5-yl)-1H-pyrrolo[2,3-b]pyridine [921];
3-(3-(1-methyl-1H-pyrazol-4-yl)pyrazolo[1,5-a]pyridin-5-yl)-5-phenyl-1H-pyrrolo[2,3-b]pyridine [922];
5-phenyl-3-(3-(pyridin-3-yl)pyrazolo[1,5-a]pyridin-5-yl)-1H-pyrrolo[2,3-b]pyridine [923];
N-(2,2-difluoroethyl)-5-(5-phenyl-1H-pyrrolo[2,3-b]pyridin-3-yl)pyrazolo[1,5-a]pyridine-3-carboxamide [924];
(R)-5-(5-phenyl-1H-pyrrolo[2,3-b]pyridin-3-yl)-N-(1,1,1-trifluoropropan-2-yl)pyrazolo[1,5-a]pyridine-3-carboxamide [925];
N-(1-methylpiperidin-4-yl)-5-(5-phenyl-1H-pyrrolo[2,3-b]pyridin-3-yl)pyrazolo[1,5-a]pyridine-3-carboxamide [926];
5-(5-phenyl-1H-pyrrolo[2,3-b]pyridin-3-yl)-N-(pyridin-3-yl)pyrazolo[1,5-a]pyridine-3-carboxamide [927];
(5-(5-phenyl-1H-pyrrolo[2,3-b]pyridin-3-yl)pyrazolo[1,5-a]pyridin-3-yl)(piperidin-1-yl)methanone [928];
(4-methylpiperazin-1-yl)(5-(5-phenyl-1H-pyrrolo[2,3-b]pyridin-3-yl)pyrazolo[1,5-a]pyridin-3-yl)methanone [929];
morpholino(5-(5-phenyl-1H-pyrrolo[2,3-b]pyridin-3-yl)pyrazolo[1,5-a]pyridin-3-yl)methanone [930];
6-(5-phenyl-1H-pyrrolo[2,3-b]pyridin-3-yl)spiro[indene-1,4′-piperidin]-3(2H)-one [932];
1′-methyl-6-(5-phenyl-1H-pyrrolo[2,3-b]pyridin-3-yl)spiro[indene-1,4′-piperidin]-3(2H)-one [933];
5-(5-phenyl-1H-pyrrolo[2,3-b]pyridin-3-yl)isoindolin-1-one [934];
3,3-dimethyl-5-(5-phenyl-1H-pyrrolo[2,3-b]pyridin-3-yl)isoindolin-1-one [935];
6′-(5-phenyl-1H-pyrrolo[2,3-b]pyridin-3-yl)spiro[cyclohexane-1,1′-isoindolin]-3′-one [936];
3-(chroman-6-yl)-5-phenyl-1H-pyrrolo[2,3-b]pyridine [937];
6-(5-phenyl-1H-pyrrolo[2,3-b]pyridin-3-yl)-3,4-dihydroisoquinolin-1(2H)-one [938];
4,4-dimethyl-6-(5-phenyl-1H-pyrrolo[2,3-b]pyridin-3-yl)-3,4-dihydroisoquinolin-1(2H)-one [939];
7-(5-phenyl-1H-pyrrolo[2,3-b]pyridin-3-yl)spiro[chromane-2,4′-piperidin]-4-one [940];
8-(5-phenyl-1H-pyrrolo[2,3-b]pyridin-3-yl)-3,4-dihydrobenzo[f][1,4]oxazepin-5(2H)-one [941];
6-(5-phenyl-1H-pyrrolo[2,3-b]pyridin-3-yl)quinoline [942];
6-(5-phenyl-1H-pyrrolo[2,3-b]pyridin-3-yl)quinoxaline [943];
6-(5-phenyl-1H-pyrrolo[2,3-b]pyridin-3-yl)quinazoline [944];
4-methoxy-6-(5-phenyl-1H-pyrrolo[2,3-b]pyridin-3-yl)quinazoline [945];
4-(4-methylpiperazin-1-yl)-6-(5-phenyl-1H-pyrrolo[2,3-b]pyridin-3-yl)quinazoline [946];
4-((1-methylpiperidin-4-yl)oxy)-6-(5-phenyl-1H-pyrrolo[2,3-b]pyridin-3-yl)quinazoline [947];
N-(1-methylpiperidin-4-yl)-6-(5-phenyl-1H-pyrrolo[2,3-b]pyridin-3-yl)quinazolin-4-amine [948];
2-methyl-5-(5-(o-tolyl)-1H-pyrrolo[2,3-b]pyridin-3-yl)-2H-indazole [949];
2-(5-(o-tolyl)-1H-pyrrolo[2,3-b]pyridin-3-yl)thieno[2,3-c]pyridine [950];
7-(5-(o-tolyl)-1H-pyrrolo[2,3-b]pyridin-3-yl)-3,4-dihydropyrrolo[1,2-a]pyrazin-1(2H)-one [951];
6-(5-(o-tolyl)-1H-pyrrolo[2,3-b]pyridin-3-yl)imidazo[1,2-a]pyridine [952];
N-(2,2-difluoroethyl)-6-(5-(o-tolyl)-1H-pyrrolo[2,3-b]pyridin-3-yl)imidazo[1,2-a]pyridine-3-carboxamide [953];
6-(5-(o-tolyl)-1H-pyrrolo[2,3-b]pyridin-3-yl)imidazo[1,2-a]pyrimidine [954];
4-fluoro-1-isopropyl-2-methyl-6-(5-(o-tolyl)-1H-pyrrolo[2,3-b]pyridin-3-yl)-1H-benzo[d]imidazole [955];
4-fluoro-2-methyl-1-(1-methylpiperidin-4-yl)-6-(5-(o-tolyl)-1H-pyrrolo[2,3-b]pyridin-3-yl)-1H-benzo[d]imidazole [956];
1-isopropyl-2-methyl-6-(5-(o-tolyl)-1H-pyrrolo[2,3-b]pyridin-3-yl)-1H-imidazo[4,5-b]pyridine [957];
1-isopropyl-2-methyl-6-(5-(o-tolyl)-1H-pyrrolo[2,3-b]pyridin-3-yl)-1H-imidazo[4,5-c]pyridine [958];
6-(5-(o-tolyl)-1H-pyrrolo[2,3-b]pyridin-3-yl)-[1,2,4]triazolo[1,5-a]pyridine [959];
7-(5-(o-tolyl)-1H-pyrrolo[2,3-b]pyridin-3-yl)-[1,2,4]triazolo[1,5-a]pyridine [960];
1-methyl-6-(5-(o-tolyl)-1H-pyrrolo[2,3-b]pyridin-3-yl)-1H-benzo[d][1,2,3]triazole [961];
6-(5-(o-tolyl)-1H-pyrrolo[2,3-b]pyridin-3-yl)imidazo[1,2-b]pyridazine [962];
3-(pyrazoN-(2,2-difluoroethyl)-5-(5-(o-tolyl)-1H-pyrrolo[2,3-b]pyridin-3-yl)pyrazolo[1,5-a]pyridine-3-carboxamide lo[1,5-a]pyridin-5-yl)-5-(o-tolyl)-1H-pyrrolo[2,3-b]pyridine [963];
3-(3-(1-methyl-1H-pyrazol-4-yl)pyrazolo[1,5-a]pyridin-5-yl)-5-(o-tolyl)-1H-pyrrolo[2,3-b]pyridine [964];
3-(3-(pyridin-3-yl)pyrazolo[1,5-a]pyridin-5-yl)-5-(o-tolyl)-1H-pyrrolo[2,3-b]pyridine [965];
N-(2,2-difluoroethyl)-5-(5-(o-tolyl)-1H-pyrrolo[2,3-b]pyridin-3-yl)pyrazolo[1,5-a]pyridine-3-carboxamide [966];
(R)-5-(5-(o-tolyl)-1H-pyrrolo[2,3-b]pyridin-3-yl)-N-(1,1,1-trifluoropropan-2-yl)pyrazolo[1,5-a]pyridine-3-carboxamide [967];
N-(1-methylpiperidin-4-yl)-5-(5-(o-tolyl)-1H-pyrrolo[2,3-b]pyridin-3-yl)pyrazolo[1,5-a]pyridine-3-carboxamide [968];
N-(pyridin-3-yl)-5-(5-(o-tolyl)-1H-pyrrolo[2,3-b]pyridin-3-yl)pyrazolo[1,5-a]pyridine-3-carboxamide [969];
piperidin-1-yl(5-(5-(o-tolyl)-1H-pyrrolo[2,3-b]pyridin-3-yl)pyrazolo[1,5-a]pyridin-3-yl)methanone [970];
(4-methylpiperazin-1-yl)(5-(5-(o-tolyl)-1H-pyrrolo[2,3-b]pyridin-3-yl)pyrazolo[1,5-a]pyridin-3-yl)methanone [971];
morpholino (5-(5-(o-tolyl)-1H-pyrrolo[2,3-b]pyridin-3-yl)pyrazolo[1,5-a]pyridin-3-yl)methanone [972];
6-(5-(o-tolyl)-1H-pyrrolo[2,3-b]pyridin-3-yl)spiro[indene-1,4′-piperidin]-3(2H)-one [974];
1′-methyl-6-(5-(o-tolyl)-1H-pyrrolo[2,3-b]pyridin-3-yl)spiro[indene-1,4′-piperidin]-3(2H)-one [975];
5-(5-(o-tolyl)-1H-pyrrolo[2,3-b]pyridin-3-yl)isoindolin-1-one [976];
3,3-dimethyl-5-(5-(o-tolyl)-1H-pyrrolo[2,3-b]pyridin-3-yl)isoindolin-1-one [977];
6′-(5-(o-tolyl)-1H-pyrrolo[2,3-b]pyridin-3-yl)spiro[cyclohexane-1,1′-isoindolin]-3′-one [978];
3-(chroman-6-yl)-5-(o-tolyl)-1H-pyrrolo[2,3-b]pyridine [979];
6-(5-(o-tolyl)-1H-pyrrolo[2,3-b]pyridin-3-yl)-3,4-dihydroisoquinolin-1(2H)-one [980];
4,4-dimethyl-6-(5-(o-tolyl)-1H-pyrrolo[2,3-b]pyridin-3-yl)-3,4-dihydroisoquinolin-1(2H)-one [981];
7-(5-(o-tolyl)-1H-pyrrolo[2,3-b]pyridin-3-yl)spiro[chromane-2,4′-piperidin]-4-one [982];
8-(5-(o-tolyl)-1H-pyrrolo[2,3-b]pyridin-3-yl)-3,4-dihydrobenzo[f][1,4]oxazepin-5(2H)-one [983];
6-(5-(o-tolyl)-1H-pyrrolo[2,3-b]pyridin-3-yl)quinoline [984];
6-(5-(o-tolyl)-1H-pyrrolo[2,3-b]pyridin-3-yl)quinoxaline [985];
6-(5-(o-tolyl)-1H-pyrrolo[2,3-b]pyridin-3-yl)quinazoline [986];
4-methoxy-6-(5-(o-tolyl)-1H-pyrrolo[2,3-b]pyridin-3-yl)quinazoline [987];
4-(4-methylpiperazin-1-yl)-6-(5-(o-tolyl)-1H-pyrrolo[2,3-b]pyridin-3-yl)quinazoline [988];
4-((1-methylpiperidin-4-yl)oxy)-6-(5-(o-tolyl)-1H-pyrrolo[2,3-b]pyridin-3-yl)quinazoline [989];
N-(1-methylpiperidin-4-yl)-6-(5-(o-tolyl)-1H-pyrrolo[2,3-b]pyridin-3-yl)quinazolin-4-amine [990];
5-(5-(3,4-difluoro-5-(piperazin-1-yl)phenyl)-1H-pyrrolo[2,3-b]pyridin-3-yl)-2-methyl-2H-indazole [991];
2-(5-(3,4-difluoro-5-(piperazin-1-yl)phenyl)-1H-pyrrolo[2,3-b]pyridin-3-yl)thieno[2,3-c]pyridine [992];
7-(5-(3,4-difluoro-5-(piperazin-1-yl)phenyl)-1H-pyrrolo[2,3-b]pyridin-3-yl)-3,4-dihydropyrrolo[1,2-a]pyrazin-1(2H)-one [993];
6-(5-(3,4-difluoro-5-(piperazin-1-yl)phenyl)-1H-pyrrolo[2,3-b]pyridin-3-yl)imidazo[1,2-a]pyridine [994];
6-(5-(3,4-difluoro-5-(piperazin-1-yl)phenyl)-1H-pyrrolo[2,3-b]pyridin-3-yl)-N-(2,2-difluoroethyl)imidazo[1,2-a]pyridine-3-carboxamide [995];
6-(5-(3,4-difluoro-5-(piperazin-1-yl)phenyl)-1H-pyrrolo[2,3-b]pyridin-3-yl)imidazo[1,2-a]pyrimidine [996];
6-(5-(3,4-difluoro-5-(piperazin-1-yl)phenyl)-1H-pyrrolo[2,3-b]pyridin-3-yl)-4-fluoro-1-isopropyl-2-methyl-1H-benzo[d]imidazole [997];
6-(5-(3,4-difluoro-5-(piperazin-1-yl)phenyl)-1H-pyrrolo[2,3-b]pyridin-3-yl)-4-fluoro-2-methyl-1-(1-methylpiperidin-4-yl)-1H-benzo[d]imidazole [998];
6-(5-(3,4-difluoro-5-(piperazin-1-yl)phenyl)-1H-pyrrolo[2,3-b]pyridin-3-yl)-1-isopropyl-2-methyl-1H-imidazo[4,5-b]pyridine [999];
6-(5-(3,4-difluoro-5-(piperazin-1-yl)phenyl)-1H-pyrrolo[2,3-b]pyridin-3-yl)-1-isopropyl-2-methyl-1H-imidazo[4,5-c]pyridine [1000];
6-(5-(3,4-difluoro-5-(piperazin-1-yl)phenyl)-1H-pyrrolo[2,3-b]pyridin-3-yl)-[1,2,4]triazolo[1,5-a]pyridine [1001];
7-(5-(3,4-difluoro-5-(piperazin-1-yl)phenyl)-1H-pyrrolo[2,3-b]pyridin-3-yl)-[1,2,4]triazolo[1,5-a]pyridine [1002];
6-(5-(3,4-difluoro-5-(piperazin-1-yl)phenyl)-1H-pyrrolo[2,3-b]pyridin-3-yl)-1-methyl-1H-benzo[d][1,2,3]triazole [1003];
6-(5-(3,4-difluoro-5-(piperazin-1-yl)phenyl)-1H-pyrrolo[2,3-b]pyridin-3-yl)imidazo[1,2-b]pyridazine [1004];
5-(3,4-difluoro-5-(piperazin-1-yl)phenyl)-3-(pyrazolo[1,5-a]pyridin-5-yl)-1H-pyrrolo[2,3-b]pyridine [1005];
5-(3,4-difluoro-5-(piperazin-1-yl)phenyl)-3-(3-(1-methyl-1H-pyrazol-4-yl)pyrazolo[1,5-a]pyridin-5-yl)-1H-pyrrolo[2,3-b]pyridine [1006];
5-(3,4-difluoro-5-(piperazin-1-yl)phenyl)-3-(3-(pyridin-3-yl)pyrazolo[1,5-a]pyridin-5-yl)-1H-pyrrolo[2,3-b]pyridine [1007];
5-(5-(3,4-difluoro-5-(piperazin-1-yl)phenyl)-1H-pyrrolo[2,3-b]pyridin-3-yl)-N-(2,2-difluoroethyl)pyrazolo[1,5-a]pyridine-3-carboxamide [1008];
(R)-5-(5-(3,4-difluoro-5-(piperazin-1-yl)phenyl)-1H-pyrrolo[2,3-b]pyridin-3-yl)-N-(1,1,1-trifluoropropan-2-yl)pyrazolo[1,5-a]pyridine-3-carboxamide [1009];
5-(5-(3,4-difluoro-5-(piperazin-1-yl)phenyl)-1H-pyrrolo[2,3-b]pyridin-3-yl)-N-(1-methylpiperidin-4-yl)pyrazolo[1,5-a]pyridine-3-carboxamide [1010];
5-(5-(3,4-difluoro-5-(piperazin-1-yl)phenyl)-1H-pyrrolo[2,3-b]pyridin-3-yl)-N-(pyridin-3-yl)pyrazolo[1,5-a]pyridine-3-carboxamide [1011];
(5-(5-(3,4-difluoro-5-(piperazin-1-yl)phenyl)-1H-pyrrolo[2,3-b]pyridin-3-yl)pyrazolo[1,5-a]pyridin-3-yl)(piperidin-1-yl)methanone [1012];
(5-(5-(3,4-difluoro-5-(piperazin-1-yl)phenyl)-1H-pyrrolo[2,3-b]pyridin-3-yl)pyrazolo[1,5-a]pyridin-3-yl)(4-methylpiperazin-1-yl)methanone [1013];
(5-(5-(3,4-difluoro-5-(piperazin-1-yl)phenyl)-1H-pyrrolo[2,3-b]pyridin-3-yl)pyrazolo[1,5-a]pyridin-3-yl)(morpholino)methanone [1014];
6-(5-(3,4-difluoro-5-(piperazin-1-yl)phenyl)-1H-pyrrolo[2,3-b]pyridin-3-yl)spiro[indene-1,4′-piperidin]-3(2H)-one [1016];
6-(5-(3,4-difluoro-5-(piperazin-1-yl)phenyl)-1H-pyrrolo[2,3-b]pyridin-3-yl)-1′-methylspiro[indene-1,4′-piperidin]-3(2H)-one [1017];
5-(5-(3,4-difluoro-5-(piperazin-1-yl)phenyl)-1H-pyrrolo[2,3-b]pyridin-3-yl)isoindolin-1-one [1018];
5-(5-(3,4-difluoro-5-(piperazin-1-yl)phenyl)-1H-pyrrolo[2,3-b]pyridin-3-yl)-3,3-dimethylisoindolin-1-one [1019];
6′-(5-(3,4-difluoro-5-(piperazin-1-yl)phenyl)-1H-pyrrolo[2,3-b]pyridin-3-yl)spiro[cyclohexane-1,1′-isoindolin]-3′-one [1020];
3-(chroman-6-yl)-5-(3,4-difluoro-5-(piperazin-1-yl)phenyl)-1H-pyrrolo[2,3-b]pyridine [1021];
6-(5-(3,4-difluoro-5-(piperazin-1-yl)phenyl)-1H-pyrrolo[2,3-b]pyridin-3-yl)-3,4-dihydroisoquinolin-1(2H)-one [1022];
6-(5-(3,4-difluoro-5-(piperazin-1-yl)phenyl)-1H-pyrrolo[2,3-b]pyridin-3-yl)-4,4-dimethyl-3,4-dihydroisoquinolin-1(2H)-one [1023];
7-(5-(3,4-difluoro-5-(piperazin-1-yl)phenyl)-1H-pyrrolo[2,3-b]pyridin-3-yl)spiro[chromane-2,4′-piperidin]-4-one [1024];
8-(5-(3,4-difluoro-5-(piperazin-1-yl)phenyl)-1H-pyrrolo[2,3-b]pyridin-3-yl)-3,4-dihydrobenzo[f][1,4]oxazepin-5(2H)-one [1025];
6-(5-(3,4-difluoro-5-(piperazin-1-yl)phenyl)-1H-pyrrolo[2,3-b]pyridin-3-yl)quinoline [1026];
6-(5-(3,4-difluoro-5-(piperazin-1-yl)phenyl)-1H-pyrrolo[2,3-b]pyridin-3-yl)quinoxaline [1027];
6-(5-(3,4-difluoro-5-(piperazin-1-yl)phenyl)-1H-pyrrolo[2,3-b]pyridin-3-yl)quinazoline [1028];
6-(5-(3,4-difluoro-5-(piperazin-1-yl)phenyl)-1H-pyrrolo[2,3-b]pyridin-3-yl)-4-methoxyquinazoline [1029];
6-(5-(3,4-difluoro-5-(piperazin-1-yl)phenyl)-1H-pyrrolo[2,3-b]pyridin-3-yl)-4-(4-methylpiperazin-1-yl)quinazoline [1030];
6-(5-(3,4-difluoro-5-(piperazin-1-yl)phenyl)-1H-pyrrolo[2,3-b]pyridin-3-yl)-4-((1-methylpiperidin-4-yl)oxy)quinazoline [1031];
6-(5-(3,4-difluoro-5-(piperazin-1-yl)phenyl)-1H-pyrrolo[2,3-b]pyridin-3-yl)-N-(1-methylpiperidin-4-yl)quinazolin-4-amine [1032];
2-methyl-5-(5-(1-methyl-1H-pyrazol-4-yl)-1H-pyrrolo[2,3-b]pyridin-3-yl)-2H-indazole [1033];
2-(5-(1-methyl-1H-pyrazol-4-yl)-1H-pyrrolo[2,3-b]pyridin-3-yl)thieno[2,3-c]pyridine [1034];
7-(5-(1-methyl-1H-pyrazol-4-yl)-1H-pyrrolo[2,3-b]pyridin-3-yl)-3,4-dihydropyrrolo[1,2-a]pyrazin-1(2H)-one [1035];
6-(5-(1-methyl-1H-pyrazol-4-yl)-1H-pyrrolo[2,3-b]pyridin-3-yl)imidazo[1,2-a]pyridine [1036];
N-(2,2-difluoroethyl)-6-(5-(1-methyl-1H-pyrazol-4-yl)-1H-pyrrolo[2,3-b]pyridin-3-yl)imidazo[1,2-a]pyridine-3-carboxamide [1037];
6-(5-(1-methyl-1H-pyrazol-4-yl)-1H-pyrrolo[2,3-b]pyridin-3-yl)imidazo[1,2-a]pyrimidine [1038];
4-fluoro-1-isopropyl-2-methyl-6-(5-(1-methyl-1H-pyrazol-4-yl)-1H-pyrrolo[2,3-b]pyridin-3-yl)-1H-benzo[d]imidazole [1039];
4-fluoro-2-methyl-6-(5-(1-methyl-1H-pyrazol-4-yl)-1H-pyrrolo[2,3-b]pyridin-3-yl)-1-(1-methylpiperidin-4-yl)-1H-benzo[d]imidazole [1040];
1-isopropyl-2-methyl-6-(5-(1-methyl-1H-pyrazol-4-yl)-1H-pyrrolo[2,3-b]pyridin-3-yl)-1H-imidazo[4,5-b]pyridine [1041];
1-isopropyl-2-methyl-6-(5-(1-methyl-1H-pyrazol-4-yl)-1H-pyrrolo[2,3-b]pyridin-3-yl)-1H-imidazo[4,5-c]pyridine [1042];
6-(5-(1-methyl-1H-pyrazol-4-yl)-1H-pyrrolo[2,3-b]pyridin-3-yl)-[1,2,4]triazolo[1,5-a]pyridine [1043];
7-(5-(1-methyl-1H-pyrazol-4-yl)-1H-pyrrolo[2,3-b]pyridin-3-yl)-[1,2,4]triazolo[1,5-a]pyridine [1044];
1-methyl-6-(5-(1-methyl-1H-pyrazol-4-yl)-1H-pyrrolo[2,3-b]pyridin-3-yl)-1H-benzo[d][1,2,3]triazole [1045];
6-(5-(1-methyl-1H-pyrazol-4-yl)-1H-pyrrolo[2,3-b]pyridin-3-yl)imidazo[1,2-b]pyridazine [1046];
5-(1-methyl-1H-pyrazol-4-yl)-3-(pyrazolo[1,5-a]pyridin-5-yl)-1H-pyrrolo[2,3-1)]pyridine [1047];
5-(1-methyl-1H-pyrazol-4-yl)-3-(3-(1-methyl-1H-pyrazol-4-yl)pyrazolo[1,5-a]pyridin-5-yl)-1H-pyrrolo[2,3-b]pyridine [1048];
5-(1-methyl-1H-pyrazol-4-yl)-3-(3-(pyridin-3-yl)pyrazolo[1,5-a]pyridin-5-yl)-1H-pyrrolo[2,3-b]pyridine [1049];
N-(2,2-difluoroethyl)-5-(5-(1-methyl-1H-pyrazol-4-yl)-1H-pyrrolo[2,3-b]pyridin-3-yl)pyrazolo[1,5-a]pyridine-3-carboxamide [1050];
(R)-5-(5-(1-methyl-1H-pyrazol-4-yl)-1H-pyrrolo[2,3-b]pyridin-3-yl)-N-(1,1,1-trifluoropropan-2-yl)pyrazolo[1,5-a]pyridine-3-carboxamide [1051];
5-(5-(1-methyl-1H-pyrazol-4-yl)-1H-pyrrolo[2,3-b]pyridin-3-yl)-N-(1-methylpiperidin-4-yl)pyrazolo[1,5-a]pyridine-3-carboxamide [1052];
5-(5-(1-methyl-1H-pyrazol-4-yl)-1H-pyrrolo[2,3-b]pyridin-3-yl)-N-(pyridin-3-yl)pyrazolo[1,5-a]pyridine-3-carboxamide [1053];
(5-(5-(1-methyl-1H-pyrazol-4-yl)-1H-pyrrolo[2,3-1)]pyridin-3-yl)pyrazolo[1,5-a]pyridin-3-yl)(piperidin-1-yl)methanone [1054];
(5-(5-(1-methyl-1H-pyrazol-4-yl)-1H-pyrrolo[2,3-1)]pyridin-3-yl)pyrazolo[1,5-a]pyridin-3-yl)(4-methylpiperazin-1-yl)methanone [1055];
(5-(5-(1-methyl-1H-pyrazol-4-yl)-1H-pyrrolo[2,3-1)]pyridin-3-yl)pyrazolo[1,5-a]pyridin-3-yl)(morpholino)methanone [1056];
5-(1-methyl-1H-pyrazol-4-yl)-3-(2-(4-methylpiperazin-1-yl)pyridin-4-yl)-1H-pyrrolo[2,3-b]pyridine [1057];
6-(5-(1-methyl-1H-pyrazol-4-yl)-1H-pyrrolo[2,3-b]pyridin-3-yl)spiro[indene-1,4′-piperidin]-3(2H)-one [1058];
1′-methyl-6-(5-(1-methyl-1H-pyrazol-4-yl)-1H-pyrrolo[2,3-b]pyridin-3-yl)spiro[indene-1,4′-piperidin]-3(2H)-one [1059];
3,3-dimethyl-5-(5-(1-methyl-1H-pyrazol-4-yl)-1H-pyrrolo[2,3-b]pyridin-3-yl)isoindolin-1-one [1061];
6′-(5-(1-methyl-1H-pyrazol-4-yl)-1H-pyrrolo[2,3-1)]pyridin-3-yl)spiro[cyclohexane-1,1′-isoindolin]-3′-one [1062];
3-(chroman-6-yl)-5-(1-methyl-1H-pyrazol-4-yl)-1H-pyrrolo[2,3-1)]pyridine [1063];
6-(5-(1-methyl-1H-pyrazol-4-yl)-1H-pyrrolo[2,3-b]pyridin-3-yl)-3,4-dihydroisoquinolin-1(2H)-one [1064];
4,4-dimethyl-6-(5-(1-methyl-1H-pyrazol-4-yl)-1H-pyrrolo[2,3-b]pyridin-3-yl)-3,4-dihydroisoquinolin-1(2H)-one [1065];
7-(5-(1-methyl-1H-pyrazol-4-yl)-1H-pyrrolo[2,3-b]pyridin-3-yl)spiro[chromane-2,4′-piperidin]-4-one [1066];
8-(5-(1-methyl-1H-pyrazol-4-yl)-1H-pyrrolo[2,3-b]pyridin-3-yl)-3,4-dihydrobenzo[f][1,4]oxazepin-5(2H)-one [1067];
6-(5-(1-methyl-1H-pyrazol-4-yl)-1H-pyrrolo[2,3-b]pyridin-3-yl)quinoline [1068];
6-(5-(1-methyl-1H-pyrazol-4-yl)-1H-pyrrolo[2,3-b]pyridin-3-yl)quinoxaline [1069];
6-(5-(1-methyl-1H-pyrazol-4-yl)-1H-pyrrolo[2,3-b]pyridin-3-yl)quinazoline [1070];
4-methoxy-6-(5-(1-methyl-1H-pyrazol-4-yl)-1H-pyrrolo[2,3-b]pyridin-3-yl)quinazoline [1071];
6-(5-(1-methyl-1H-pyrazol-4-yl)-1H-pyrrolo[2,3-b]pyridin-3-yl)-4-(4-methylpiperazin-1-yl)quinazoline [1072];
6-(5-(1-methyl-1H-pyrazol-4-yl)-1H-pyrrolo[2,3-b]pyridin-3-yl)-4-((1-methylpiperidin-4-yl)oxy)quinazoline [1073];
6-(5-(1-methyl-1H-pyrazol-4-yl)-1H-pyrrolo[2,3-b]pyridin-3-yl)-N-(1-methylpiperidin-4-yl)quinazolin-4-amine [1074];
2-methyl-5-(5-(1,3,5-trimethyl-1H-pyrazol-4-yl)-1H-pyrrolo[2,3-b]pyridin-3-yl)-2H-indazole [1075];
2-(5-(1,3,5-trimethyl-1H-pyrazol-4-yl)-1H-pyrrolo[2,3-b]pyridin-3-yl)thieno[2,3-c]pyridine [1076];
7-(5-(1,3,5-trimethyl-1H-pyrazol-4-yl)-1H-pyrrolo[2,3-b]pyridin-3-yl)-3,4-dihydropyrrolo[1,2-a]pyrazin-1(2H)-one [1077];
6-(5-(1,3,5-trimethyl-1H-pyrazol-4-yl)-1H-pyrrolo[2,3-b]pyridin-3-yl)imidazo[1,2-a]pyridine [1078];
N-(2,2-difluoroethyl)-6-(5-(1,3,5-trimethyl-1H-pyrazol-4-yl)-1H-pyrrolo[2,3-b]pyridin-3-yl)imidazo[1,2-a]pyridine-3-carboxamide [1079];
6-(5-(1,3,5-trimethyl-1H-pyrazol-4-yl)-1H-pyrrolo[2,3-b]pyridin-3-yl)imidazo[1,2-a]pyrimidine [1080];
4-fluoro-1-isopropyl-2-methyl-6-(5-(1,3,5-trimethyl-1H-pyrazol-4-yl)-1H-pyrrolo[2,3-b]pyridin-3-yl)-1H-benzo[d]imidazole [1081];
4-fluoro-2-methyl-1-(1-methylpiperidin-4-yl)-6-(5-(1,3,5-trimethyl-1H-pyrazol-4-yl)-1H-pyrrolo[2,3-b]pyridin-3-yl)-1H-benzo[d]imidazole [1082];
1-isopropyl-2-methyl-6-(5-(1,3,5-trimethyl-1H-pyrazol-4-yl)-1H-pyrrolo[2,3-b]pyridin-3-yl)-1H-imidazo[4,5-1)]pyridine [1083];
1-isopropyl-2-methyl-6-(5-(1,3,5-trimethyl-1H-pyrazol-4-yl)-1H-pyrrolo[2,3-b]pyridin-3-yl)-1H-imidazo[4,5-c]pyridine [1084];
6-(5-(1,3,5-trimethyl-1H-pyrazol-4-yl)-1H-pyrrolo[2,3-b]pyridin-3-yl)-[1,2,4]triazolo[1,5-a]pyridine [1085];
7-(5-(1,3,5-trimethyl-1H-pyrazol-4-yl)-1H-pyrrolo[2,3-b]pyridin-3-yl)-[1,2,4]triazolo[1,5-a]pyridine [1086];
1-methyl-6-(5-(1,3,5-trimethyl-1H-pyrazol-4-yl)-1H-pyrrolo[2,3-b]pyridin-3-yl)-1H-benzo[d][1,2,3]triazole [1087];
6-(5-(1,3,5-trimethyl-1H-pyrazol-4-yl)-1H-pyrrolo[2,3-b]pyridin-3-yl)imidazo[1,2-b]pyridazine [1088];
3-(pyrazolo[1,5-a]pyridin-5-yl)-5-(1,3,5-trimethyl-1H-pyrazol-4-yl)-1H-pyrrolo[2,3-1)]pyridine [1089];
3-(3-(1-methyl-1H-pyrazol-4-yl)pyrazolo[1,5-a]pyridin-5-yl)-5-(1,3,5-trimethyl-1H-pyrazol-4-yl)-1H-pyrrolo[2,3-1)]pyridine [1090];
3-(3-(pyridin-3-yl)pyrazolo[1,5-a]pyridin-5-yl)-5-(1,3,5-trimethyl-1H-pyrazol-4-yl)-1H-pyrrolo[2,3-b]pyridine [1091];
N-(2,2-difluoroethyl)-5-(5-(1,3,5-trimethyl-1H-pyrazol-4-yl)-1H-pyrrolo[2,3-b]pyridin-3-yl)pyrazolo[1,5-a]pyridine-3-carboxamide [1092];
(R)-N-(1,1,1-trifluoropropan-2-yl)-5-(5-(1,3,5-trimethyl-1H-pyrazol-4-yl)-1H-pyrrolo[2,3-b]pyridin-3-yl)pyrazolo[1,5-a]pyridine-3-carboxamide [1093];
N-(1-methylpiperidin-4-yl)-5-(5-(1,3,5-trimethyl-1H-pyrazol-4-yl)-1H-pyrrolo[2,3-b]pyridin-3-yl)pyrazolo[1,5-a]pyridine-3-carboxamide [1094];
N-(pyridin-3-yl)-5-(5-(1,3,5-trimethyl-1H-pyrazol-4-yl)-1H-pyrrolo[2,3-b]pyridin-3-yl)pyrazolo[1,5-a]pyridine-3-carboxamide [1095];
piperidin-1-yl(5-(5-(1,3,5-trimethyl-1H-pyrazol-4-yl)-1H-pyrrolo[2,3-b]pyridin-3-yl)pyrazolo[1,5-a]pyridin-3-yl)methanone [1096];
(4-methylpiperazin-1-yl)(5-(5-(1,3,5-trimethyl-1H-pyrazol-4-yl)-1H-pyrrolo[2,3-b]pyridin-3-yl)pyrazolo[1,5-a]pyridin-3-yl)methanone [1097];
morpholino(5-(5-(1,3,5-trimethyl-1H-pyrazol-4-yl)-1H-pyrrolo[2,3-b]pyridin-3-yl)pyrazolo[1,5-a]pyridin-3-yl)methanone [1098];
6-(5-(1,3,5-trimethyl-1H-pyrazol-4-yl)-1H-pyrrolo[2,3-b]pyridin-3-yl)spiro[indene-1,4′-piperidin]-3(2H)-one [1100];
1′-methyl-6-(5-(1,3,5-trimethyl-1H-pyrazol-4-yl)-1H-pyrrolo[2,3-b]pyridin-3-yl)spiro[indene-1,4′-piperidin]-3(2H)-one [1101];
5-(5-(1,3,5-trimethyl-1H-pyrazol-4-yl)-1H-pyrrolo[2,3-b]pyridin-3-yl)isoindolin-1-one [1102];
3,3-dimethyl-5-(5-(1,3,5-trimethyl-1H-pyrazol-4-yl)-1H-pyrrolo[2,3-b]pyridin-3-yl)isoindolin-1-one [1103];
6′-(5-(1,3,5-trimethyl-1H-pyrazol-4-yl)-1H-pyrrolo[2,3-b]pyridin-3-yl)spiro[cyclohexane-1,1′-isoindolin]-3′-one [1104];
3-(chroman-6-yl)-5-(1,3,5-trimethyl-1H-pyrazol-4-yl)-1H-pyrrolo[2,3-b]pyridine [1105];
6-(5-(1,3,5-trimethyl-1H-pyrazol-4-yl)-1H-pyrrolo[2,3-b]pyridin-3-yl)-3,4-dihydroisoquinolin-1(2H)-one [1106];
4,4-dimethyl-6-(5-(1,3,5-trimethyl-1H-pyrazol-4-yl)-1H-pyrrolo[2,3-b]pyridin-3-yl)-3,4-dihydroisoquinolin-1(2H)-one [1107];
7-(5-(1,3,5-trimethyl-1H-pyrazol-4-yl)-1H-pyrrolo[2,3-b]pyridin-3-yl)spiro[chromane-2,4′-piperidin]-4-one [1108];
8-(5-(1,3,5-trimethyl-1H-pyrazol-4-yl)-1H-pyrrolo[2,3-b]pyridin-3-yl)-3,4-dihydrobenzo[f][1,4]oxazepin-5(2H)-one [1109];
6-(5-(1,3,5-trimethyl-1H-pyrazol-4-yl)-1H-pyrrolo[2,3-b]pyridin-3-yl)quinoline [1110];
6-(5-(1,3,5-trimethyl-1H-pyrazol-4-yl)-1H-pyrrolo[2,3-b]pyridin-3-yl)quinoxaline [1111];
6-(5-(1,3,5-trimethyl-1H-pyrazol-4-yl)-1H-pyrrolo[2,3-b]pyridin-3-yl)quinazoline [1112];
4-methoxy-6-(5-(1,3,5-trimethyl-1H-pyrazol-4-yl)-1H-pyrrolo[2,3-b]pyridin-3-yl)quinazoline [1113];
4-(4-methylpiperazin-1-yl)-6-(5-(1,3,5-trimethyl-1H-pyrazol-4-yl)-1H-pyrrolo[2,3-b]pyridin-3-yl)quinazoline [1114];
4-((1-methylpiperidin-4-yl)oxy)-6-(5-(1,3,5-trimethyl-1H-pyrazol-4-yl)-1H-pyrrolo[2,3-b]pyridin-3-yl)quinazoline [1115];
N-(1-methylpiperidin-4-yl)-6-(5-(1,3,5-trimethyl-1H-pyrazol-4-yl)-1H-pyrrolo[2,3-b]pyridin-3-yl)quinazolin-4-amine [1116];
5-methyl-3-(3-(2-methyl-2H-indazol-5-yl)-1H-pyrrolo[2,3-b]pyridin-5-yl)-4,5,6,7-tetrahydropyrazolo[1,5-a]pyrazine [1117];
2-(5-(5-methyl-4,5,6,7-tetrahydropyrazolo[1,5-a]pyrazin-3-yl)-1H-pyrrolo[2,3-b]pyridin-3-yl)thieno[2,3-c]pyridine [1118];
7-(5-(5-methyl-4,5,6,7-tetrahydropyrazolo[1,5-a]pyrazin-3-yl)-1H-pyrrolo[2,3-b]pyridin-3-yl)-3,4-dihydropyrrolo[1,2-a]pyrazin-1(2H)-one [1119];
3-(3-(imidazo[1,2-a]pyridin-6-yl)-1H-pyrrolo[2,3-b]pyridin-5-yl)-5-methyl-4,5,6,7-tetrahydropyrazolo[1,5-a]pyrazine [1120];
N-(2,2-difluoroethyl)-6-(5-(5-methyl-4,5,6,7-tetrahydropyrazolo[1,5-a]pyrazin-3-yl)-1H-pyrrolo[2,3-b]pyridin-3-yl)imidazo[1,2-a]pyridine-3-carboxamide [1121];
6-(5-(5-methyl-4,5,6,7-tetrahydropyrazolo[1,5-a]pyrazin-3-yl)-1H-pyrrolo[2,3-b]pyridin-3-yl)imidazo[1,2-a]pyrimidine [1122];
3-(3-(4-fluoro-1-isopropyl-2-methyl-1H-benzo[d]imidazol-6-yl)-1H-pyrrolo[2,3-b]pyridin-5-yl)-5-methyl-4,5,6,7-tetrahydropyrazolo[1,5-a]pyrazine [1123];
3-(3-(4-fluoro-2-methyl-1-(1-methylpiperidin-4-yl)-1H-benzo[d]imidazol-6-yl)-1H-pyrrolo[2,3-b]pyridin-5-yl)-5-methyl-4,5,6,7-tetrahydropyrazolo[1,5-a]pyrazine [1124];
1-isopropyl-2-methyl-6-(5-(5-methyl-4,5,6,7-tetrahydropyrazolo[1,5-a]pyrazin-3-yl)-1H-pyrrolo[2,3-b]pyridin-3-yl)-1H-imidazo[4,5-b]pyridine [1125];
1-isopropyl-2-methyl-6-(5-(5-methyl-4,5,6,7-tetrahydropyrazolo[1,5-a]pyrazin-3-yl)-1H-pyrrolo[2,3-b]pyridin-3-yl)-1H-imidazo[4,5-c]pyridine [1126];
6-(5-(5-methyl-4,5,6,7-tetrahydropyrazolo[1,5-a]pyrazin-3-yl)-1H-pyrrolo[2,3-b]pyridin-3-yl)-[1,2,4]triazolo[1,5-a]pyridine [1127];
7-(5-(5-methyl-4,5,6,7-tetrahydropyrazolo[1,5-a]pyrazin-3-yl)-1H-pyrrolo[2,3-b]pyridin-3-yl)-[1,2,4]triazolo[1,5-a]pyridine [1128];
1-methyl-6-(5-(5-methyl-4,5,6,7-tetrahydropyrazolo[1,5-a]pyrazin-3-yl)-1H-pyrrolo[2,3-b]pyridin-3-yl)-1H-benzo[d][1,2,3]triazole [1129];
6-(5-(5-methyl-4,5,6,7-tetrahydropyrazolo[1,5-a]pyrazin-3-yl)-1H-pyrrolo[2,3-b]pyridin-3-yl)imidazo[1,2-b]pyridazine [1130];
5-methyl-3-(3-(pyrazolo[1,5-a]pyridin-5-yl)-1H-pyrrolo[2,3-b]pyridin-5-yl)-4,5,6,7-tetrahydropyrazolo[1,5-a]pyrazine [1131];
5-methyl-3-(3-(3-(1-methyl-1H-pyrazol-4-yl)pyrazolo[1,5-a]pyridin-5-yl)-1H-pyrrolo[2,3-b]pyridin-5-yl)-4,5,6,7-tetrahydropyrazolo[1,5-a]pyrazine [1132];
5-methyl-3-(3-(3-(pyridin-3-yl)pyrazolo[1,5-a]pyridin-5-yl)-1H-pyrrolo[2,3-b]pyridin-5-yl)-4,5,6,7-tetrahydropyrazolo[1,5-a]pyrazine [1133];
N-(2,2-difluoroethyl)-5-(5-(5-methyl-4,5,6,7-tetrahydropyrazolo[1,5-a]pyrazin-3-yl)-1H-pyrrolo[2,3-b]pyridin-3-yl)pyrazolo[1,5-a]pyridine-3-carboxamide [1134];
(R)-5-(5-(5-methyl-4,5,6,7-tetrahydropyrazolo[1,5-a]pyrazin-3-yl)-1H-pyrrolo[2,3-b]pyridin-3-yl)-N-(1,1,1-trifluoropropan-2-yl)pyrazolo[1,5-a]pyridine-3-carboxamide [1135];
5-(5-(5-methyl-4,5,6,7-tetrahydropyrazolo[1,5-a]pyrazin-3-yl)-1H-pyrrolo[2,3-b]pyridin-3-yl)-N-(1-methylpiperidin-4-yl)pyrazolo[1,5-a]pyridine-3-carboxamide [1136];
5-(5-(5-methyl-4,5,6,7-tetrahydropyrazolo[1,5-a]pyrazin-3-yl)-1H-pyrrolo[2,3-b]pyridin-3-yl)-N-(pyridin-3-yl)pyrazolo[1,5-a]pyridine-3-carboxamide [1137];
(5-(5-(5-methyl-4,5,6,7-tetrahydropyrazolo[1,5-a]pyrazin-3-yl)-1H-pyrrolo[2,3-b]pyridin-3-yl)pyrazolo[1,5-a]pyridin-3-yl)(piperidin-1-yl)methanone [1138];
(5-(5-(5-methyl-4,5,6,7-tetrahydropyrazolo[1,5-a]pyrazin-3-yl)-1H-pyrrolo[2,3-b]pyridin-3-yl)pyrazolo[1,5-a]pyridin-3-yl)(4-methylpiperazin-1-yl)methanone [1139];
(5-(5-(5-methyl-4,5,6,7-tetrahydropyrazolo[1,5-a]pyrazin-3-yl)-1H-pyrrolo[2,3-b]pyridin-3-yl)pyrazolo[1,5-a]pyridin-3-yl)(morpholino)methanone [1140];
6-(5-(5-methyl-4,5,6,7-tetrahydropyrazolo[1,5-a]pyrazin-3-yl)-1H-pyrrolo[2,3-b]pyridin-3-yl)spiro[indene-1,4′-piperidin]-3(2H)-one [1142];
1′-methyl-6-(5-(5-methyl-4,5,6,7-tetrahydropyrazolo[1,5-a]pyrazin-3-yl)-1H-pyrrolo[2,3-b]pyridin-3-yl)spiro[indene-1,4′-piperidin]-3(2H)-one [1143];
5-(5-(5-methyl-4,5,6,7-tetrahydropyrazolo[1,5-a]pyrazin-3-yl)-1H-pyrrolo[2,3-b]pyridin-3-yl)isoindolin-1-one [1144];
3,3-dimethyl-5-(5-(5-methyl-4,5,6,7-tetrahydropyrazolo[1,5-a]pyrazin-3-yl)-1H-pyrrolo[2,3-b]pyridin-3-yl)isoindolin-1-one [1145];
6′-(5-(5-methyl-4,5,6,7-tetrahydropyrazolo[1,5-a]pyrazin-3-yl)-1H-pyrrolo[2,3-b]pyridin-3-yl)spiro[cyclohexane-1,1′-isoindolin]-3′-one [1146];
3-(3-(chroman-6-yl)-1H-pyrrolo[2,3-b]pyridin-5-yl)-5-methyl-4,5,6,7-tetrahydropyrazolo[1,5-a]pyrazine [1147];
6-(5-(5-methyl-4,5,6,7-tetrahydropyrazolo[1,5-a]pyrazin-3-yl)-1H-pyrrolo[2,3-b]pyridin-3-yl)-3,4-dihydroisoquinolin-1(2H)-one [1148];
4,4-dimethyl-6-(5-(5-methyl-4,5,6,7-tetrahydropyrazolo[1,5-a]pyrazin-3-yl)-1H-pyrrolo[2,3-b]pyridin-3-yl)-3,4-dihydroisoquinolin-1(2H)-one [1149];
7-(5-(5-methyl-4,5,6,7-tetrahydropyrazolo[1,5-a]pyrazin-3-yl)-1H-pyrrolo[2,3-b]pyridin-3-yl)spiro[chromane-2,4′-piperidin]-4-one [1150];
8-(5-(5-methyl-4,5,6,7-tetrahydropyrazolo[1,5-a]pyrazin-3-yl)-1H-pyrrolo[2,3-b]pyridin-3-yl)-3,4-dihydrobenzo[f][1,4]oxazepin-5(2H)-one [1151];
6-(5-(5-methyl-4,5,6,7-tetrahydropyrazolo[1,5-a]pyrazin-3-yl)-1H-pyrrolo[2,3-b]pyridin-3-yl)quinoline [1152];
6-(5-(5-methyl-4,5,6,7-tetrahydropyrazolo[1,5-a]pyrazin-3-yl)-1H-pyrrolo[2,3-b]pyridin-3-yl)quinoxaline [1153];
6-(5-(5-methyl-4,5,6,7-tetrahydropyrazolo[1,5-a]pyrazin-3-yl)-1H-pyrrolo[2,3-b]pyridin-3-yl)quinazoline [1154];
4-methoxy-6-(5-(5-methyl-4,5,6,7-tetrahydropyrazolo[1,5-a]pyrazin-3-yl)-1H-pyrrolo[2,3-b]pyridin-3-yl)quinazoline [1155];
6-(5-(5-methyl-4,5,6,7-tetrahydropyrazolo[1,5-a]pyrazin-3-yl)-1H-pyrrolo[2,3-b]pyridin-3-yl)-4-(4-methylpiperazin-1-yl)quinazoline [1156];
6-(5-(5-methyl-4,5,6,7-tetrahydropyrazolo[1,5-a]pyrazin-3-yl)-1H-pyrrolo[2,3-b]pyridin-3-yl)-4-((1-methylpiperidin-4-yl)oxy)quinazoline [1157];
6-(5-(5-methyl-4,5,6,7-tetrahydropyrazolo[1,5-a]pyrazin-3-yl)-1H-pyrrolo[2,3-b]pyridin-3-yl)-N-(1-methylpiperidin-4-yl)quinazolin-4-amine [1158];
2-methyl-5-(5-(pyridin-3-yl)-1H-pyrrolo[2,3-b]pyridin-3-yl)-2H-indazole [1159];
2-(5-(pyridin-3-yl)-1H-pyrrolo[2,3-b]pyridin-3-yl)thieno[2,3-c]pyridine [1160];
7-(5-(pyridin-3-yl)-1H-pyrrolo[2,3-b]pyridin-3-yl)-3,4-dihydropyrrolo[1,2-a]pyrazin-1(2H)-one [1161];
6-(5-(pyridin-3-yl)-1H-pyrrolo[2,3-b]pyridin-3-yl)imidazo[1,2-a]pyridine [1162];
N-(2,2-difluoroethyl)-6-(5-(pyridin-3-yl)-1H-pyrrolo[2,3-b]pyridin-3-yl)imidazo[1,2-a]pyridine-3-carboxamide [1163];
6-(5-(pyridin-3-yl)-1H-pyrrolo[2,3-b]pyridin-3-yl)imidazo[1,2-a]pyrimidine [1164];
4-fluoro-1-isopropyl-2-methyl-6-(5-(pyridin-3-yl)-1H-pyrrolo[2,3-b]pyridin-3-yl)-1H-benzo[d]imidazole [1165];
4-fluoro-2-methyl-1-(1-methylpiperidin-4-yl)-6-(5-(pyridin-3-yl)-1H-pyrrolo[2,3-b]pyridin-3-yl)-1H-benzo[d]imidazole [1166];
1-isopropyl-2-methyl-6-(5-(pyridin-3-yl)-1H-pyrrolo[2,3-b]pyridin-3-yl)-1H-imidazo[4,5-b]pyridine [1167];
1-isopropyl-2-methyl-6-(5-(pyridin-3-yl)-1H-pyrrolo[2,3-b]pyridin-3-yl)-1H-imidazo[4,5-c]pyridine [1168];
6-(5-(pyridin-3-yl)-1H-pyrrolo[2,3-b]pyridin-3-yl)-[1,2,4]triazolo[1,5-a]pyridine [1169];
7-(5-(pyridin-3-yl)-1H-pyrrolo[2,3-b]pyridin-3-yl)-[1,2,4]triazolo[1,5-a]pyridine [1170];
1-methyl-6-(5-(pyridin-3-yl)-1H-pyrrolo[2,3-b]pyridin-3-yl)-1H-benzo[d][1,2,3]triazole [1171];
6-(5-(pyridin-3-yl)-1H-pyrrolo[2,3-b]pyridin-3-yl)imidazo[1,2-b]pyridazine [1172];
3-(pyrazolo[1,5-a]pyridin-5-yl)-5-(pyridin-3-yl)-1H-pyrrolo[2,3-b]pyridine [1173];
3-(3-(1-methyl-1H-pyrazol-4-yl)pyrazolo[1,5-a]pyridin-5-yl)-5-(pyridin-3-yl)-1H-pyrrolo[2,3-b]pyridine [1174];
5-(pyridin-3-yl)-3-(3-(pyridin-3-yl)pyrazolo[1,5-a]pyridin-5-yl)-1H-pyrrolo[2,3-b]pyridine [1175];
N-(2,2-difluoroethyl)-5-(5-(pyridin-3-yl)-1H-pyrrolo[2,3-b]pyridin-3-yl)pyrazolo[1,5-a]pyridine-3-carboxamide [1176];
(R)-5-(5-(pyridin-3-yl)-1H-pyrrolo[2,3-b]pyridin-3-yl)-N-(1,1,1-trifluoropropan-2-yl)pyrazolo[1,5-a]pyridine-3-carboxamide [1177];
N-(1-methylpiperidin-4-yl)-5-(5-(pyridin-3-yl)-1H-pyrrolo[2,3-b]pyridin-3-yl)pyrazolo[1,5-a]pyridine-3-carboxamide [1178];
N-(pyridin-3-yl)-5-(5-(pyridin-3-yl)-1H-pyrrolo[2,3-b]pyridin-3-yl)pyrazolo[1,5-a]pyridine-3-carboxamide [1179];
piperidin-1-yl(5-(5-(pyridin-3-yl)-1H-pyrrolo[2,3-b]pyridin-3-yl)pyrazolo[1,5-a]pyridin-3-yl)methanone [1180];
(4-methylpiperazin-1-yl)(5-(5-(pyridin-3-yl)-1H-pyrrolo[2,3-b]pyridin-3-yl)pyrazolo[1,5-a]pyridin-3-yl)methanone [1181];
morpholino(5-(5-(pyridin-3-yl)-1H-pyrrolo[2,3-b]pyridin-3-yl)pyrazolo[1,5-a]pyridin-3-yl)methanone [1182];
6-(5-(pyridin-3-yl)-1H-pyrrolo[2,3-b]pyridin-3-yl)spiro[indene-1,4′-piperidin]-3(2H)-one [1184];
1′-methyl-6-(5-(pyridin-3-yl)-1H-pyrrolo[2,3-b]pyridin-3-yl)spiro[indene-1,4′-piperidin]-3(2H)-one [1185];
5-(5-(pyridin-3-yl)-1H-pyrrolo[2,3-b]pyridin-3-yl)isoindolin-1-one [1186];
3,3-dimethyl-5-(5-(pyridin-3-yl)-1H-pyrrolo[2,3-b]pyridin-3-yl)isoindolin-1-one [1187];
6′-(5-(pyridin-3-yl)-1H-pyrrolo[2,3-b]pyridin-3-yl)spiro[cyclohexane-1,1′-isoindolin]-3′-one [1188];
3-(chroman-6-yl)-5-(pyridin-3-yl)-1H-pyrrolo[2,3-b]pyridine [1189];
6-(5-(pyridin-3-yl)-1H-pyrrolo[2,3-b]pyridin-3-yl)-3,4-dihydroisoquinolin-1(2H)-one [1190];
4,4-dimethyl-6-(5-(pyridin-3-yl)-1H-pyrrolo[2,3-b]pyridin-3-yl)-3,4-dihydroisoquinolin-1(2H)-one [1191];
7-(5-(pyridin-3-yl)-1H-pyrrolo[2,3-b]pyridin-3-yl)spiro[chromane-2,4′-piperidin]-4-one [1192];
8-(5-(pyridin-3-yl)-1H-pyrrolo[2,3-b]pyridin-3-yl)-3,4-dihydrobenzo[f][1,4]oxazepin-5(2H)-one [1193];
6-(5-(pyridin-3-yl)-1H-pyrrolo[2,3-b]pyridin-3-yl)quinoline [1194];
6-(5-(pyridin-3-yl)-1H-pyrrolo[2,3-b]pyridin-3-yl)quinoxaline [1195];
6-(5-(pyridin-3-yl)-1H-pyrrolo[2,3-b]pyridin-3-yl)quinazoline [1196];
4-methoxy-6-(5-(pyridin-3-yl)-1H-pyrrolo[2,3-b]pyridin-3-yl)quinazoline [1197];
4-(4-methylpiperazin-1-yl)-6-(5-(pyridin-3-yl)-1H-pyrrolo[2,3-b]pyridin-3-yl)quinazoline [1198];
4-((1-methylpiperidin-4-yl)oxy)-6-(5-(pyridin-3-yl)-1H-pyrrolo[2,3-b]pyridin-3-yl)quinazoline [1199];
N-(1-methylpiperidin-4-yl)-6-(5-(pyridin-3-yl)-1H-pyrrolo[2,3-b]pyridin-3-yl)quinazolin-4-amine [1200];
2-methyl-5-(5-(2-methylpyridin-3-yl)-1H-pyrrolo[2,3-b]pyridin-3-yl)-2H-indazole [1201];
2-(5-(2-methylpyridin-3-yl)-1H-pyrrolo[2,3-b]pyridin-3-yl)thieno[2,3-c]pyridine [1202];
7-(5-(2-methylpyridin-3-yl)-1H-pyrrolo[2,3-b]pyridin-3-yl)-3,4-dihydropyrrolo[1,2-a]pyrazin-1(2H)-one [1203];
6-(5-(2-methylpyridin-3-yl)-1H-pyrrolo[2,3-b]pyridin-3-yl)imidazo[1,2-a]pyridine [1204];
N-(2,2-difluoroethyl)-6-(5-(2-methylpyridin-3-yl)-1H-pyrrolo[2,3-b]pyridin-3-yl)imidazo[1,2-a]pyridine-3-carboxamide [1205];
6-(5-(2-methylpyridin-3-yl)-1H-pyrrolo[2,3-b]pyridin-3-yl)imidazo[1,2-a]pyrimidine [1206];
4-fluoro-1-isopropyl-2-methyl-6-(5-(2-methylpyridin-3-yl)-1H-pyrrolo[2,3-b]pyridin-3-yl)-1H-benzo[d]imidazole [1207];
4-fluoro-2-methyl-1-(1-methylpiperidin-4-yl)-6-(5-(2-methylpyridin-3-yl)-1H-pyrrolo[2,3-b]pyridin-3-yl)-1H-benzo[d]imidazole [1208];
1-isopropyl-2-methyl-6-(5-(2-methylpyridin-3-yl)-1H-pyrrolo[2,3-b]pyridin-3-yl)-1H-imidazo[4,5-b]pyridine [1209];
1-isopropyl-2-methyl-6-(5-(2-methylpyridin-3-yl)-1H-pyrrolo[2,3-b]pyridin-3-yl)-1H-imidazo[4,5-c]pyridine [1210];
6-(5-(2-methylpyridin-3-yl)-1H-pyrrolo[2,3-b]pyridin-3-yl)-[1,2,4]triazolo[1,5-a]pyridine [1211];
7-(5-(2-methylpyridin-3-yl)-1H-pyrrolo[2,3-b]pyridin-3-yl)-[1,2,4]triazolo[1,5-a]pyridine [1212];
1-methyl-6-(5-(2-methylpyridin-3-yl)-1H-pyrrolo[2,3-b]pyridin-3-yl)-1H-benzo[d][1,2,3]triazole [1213];
6-(5-(2-methylpyridin-3-yl)-1H-pyrrolo[2,3-b]pyridin-3-yl)imidazo[1,2-b]pyridazine [1214];
5-(2-methylpyridin-3-yl)-3-(pyrazolo[1,5-a]pyridin-5-yl)-1H-pyrrolo[2,3-b]pyridine [1215];
3-(3-(1-methyl-1H-pyrazol-4-yl)pyrazolo[1,5-a]pyridin-5-yl)-5-(2-methylpyridin-3-yl)-1H-pyrrolo[2,3-b]pyridine [1216];
5-(2-methylpyridin-3-yl)-3-(3-(pyridin-3-yl)pyrazolo[1,5-a]pyridin-5-yl)-1H-pyrrolo[2,3-b]pyridine [1217];
N-(2,2-difluoroethyl)-5-(5-(2-methylpyridin-3-yl)-1H-pyrrolo[2,3-b]pyridin-3-yl)pyrazolo[1,5-a]pyridine-3-carboxamide [1218];
(R)-5-(5-(2-methylpyridin-3-yl)-1H-pyrrolo[2,3-b]pyridin-3-yl)-N-(1,1,1-trifluoropropan-2-yl)pyrazolo[1,5-a]pyridine-3-carboxamide [1219];
N-(1-methylpiperidin-4-yl)-5-(5-(2-methylpyridin-3-yl)-1H-pyrrolo[2,3-b]pyridin-3-yl)pyrazolo[1,5-a]pyridine-3-carboxamide [1220];
5-(5-(2-methylpyridin-3-yl)-1H-pyrrolo[2,3-b]pyridin-3-yl)-N-(pyridin-3-yl)pyrazolo[1,5-a]pyridine-3-carboxamide [1221];
(5-(5-(2-methylpyridin-3-yl)-1H-pyrrolo[2,3-b]pyridin-3-yl)pyrazolo[1,5-a]pyridin-3-yl)(piperidin-1-yl)methanone [1222];
(4-methylpiperazin-1-yl)(5-(5-(2-methylpyridin-3-yl)-1H-pyrrolo[2,3-b]pyridin-3-yl)pyrazolo[1,5-a]pyridin-3-yl)methanone [1223];
(5-(5-(2-methylpyridin-3-yl)-1H-pyrrolo[2,3-b]pyridin-3-yl)pyrazolo[1,5-a]pyridin-3-yl)(morpholino)methanone [1224];
6-(5-(2-methylpyridin-3-yl)-1H-pyrrolo[2,3-b]pyridin-3-yl)spiro[indene-1,4′-piperidin]-3(2H)-one [1226];
1′-methyl-6-(5-(2-methylpyridin-3-yl)-1H-pyrrolo[2,3-b]pyridin-3-yl)spiro[indene-1,4′-piperidin]-3(2H)-one [1227];
5-(5-(2-methylpyridin-3-yl)-1H-pyrrolo[2,3-b]pyridin-3-yl)isoindolin-1-one [1228];
3,3-dimethyl-5-(5-(2-methylpyridin-3-yl)-1H-pyrrolo[2,3-b]pyridin-3-yl)isoindolin-1-one [1229];
6′-(5-(2-methylpyridin-3-yl)-1H-pyrrolo[2,3-b]pyridin-3-yl)spiro[cyclohexane-1,1′-isoindolin]-3′-one [1230];
3-(chroman-6-yl)-5-(2-methylpyridin-3-yl)-1H-pyrrolo[2,3-b]pyridine [1231];
6-(5-(2-methylpyridin-3-yl)-1H-pyrrolo[2,3-b]pyridin-3-yl)-3,4-dihydroisoquinolin-1(2H)-one [1232];
4,4-dimethyl-6-(5-(2-methylpyridin-3-yl)-1H-pyrrolo[2,3-b]pyridin-3-yl)-3,4-dihydroisoquinolin-1(2H)-one [1233];
7-(5-(2-methylpyridin-3-yl)-1H-pyrrolo[2,3-b]pyridin-3-yl)spiro[chromane-2,4′-piperidin]-4-one [1234];
8-(5-(2-methylpyridin-3-yl)-1H-pyrrolo[2,3-b]pyridin-3-yl)-3,4-dihydrobenzo[f][1,4]oxazepin-5(2H)-one [1235];
6-(5-(2-methylpyridin-3-yl)-1H-pyrrolo[2,3-b]pyridin-3-yl)quinoline [1236];
6-(5-(2-methylpyridin-3-yl)-1H-pyrrolo[2,3-b]pyridin-3-yl)quinoxaline [1237];
6-(5-(2-methylpyridin-3-yl)-1H-pyrrolo[2,3-b]pyridin-3-yl)quinazoline [1238];
4-methoxy-6-(5-(2-methylpyridin-3-yl)-1H-pyrrolo[2,3-b]pyridin-3-yl)quinazoline [1239];
4-(4-methylpiperazin-1-yl)-6-(5-(2-methylpyridin-3-yl)-1H-pyrrolo[2,3-b]pyridin-3-yl)quinazoline [1240];
4-((1-methylpiperidin-4-yl)oxy)-6-(5-(2-methylpyridin-3-yl)-1H-pyrrolo[2,3-b]pyridin-3-yl)quinazoline [1241];
N-(1-methylpiperidin-4-yl)-6-(5-(2-methylpyridin-3-yl)-1H-pyrrolo[2,3-b]pyridin-3-yl)quinazolin-4-amine [1242];
2-methyl-5-(5-(4-methylpyridin-3-yl)-1H-pyrrolo[2,3-b]pyridin-3-yl)-2H-indazole [1243];
2-(5-(4-methylpyridin-3-yl)-1H-pyrrolo[2,3-b]pyridin-3-yl)thieno[2,3-c]pyridine [1244];
7-(5-(4-methylpyridin-3-yl)-1H-pyrrolo[2,3-b]pyridin-3-yl)-3,4-dihydropyrrolo[1,2-a]pyrazin-1(2H)-one [1245];
6-(5-(4-methylpyridin-3-yl)-1H-pyrrolo[2,3-b]pyridin-3-yl)imidazo[1,2-a]pyridine [1246];
N-(2,2-difluoroethyl)-6-(5-(4-methylpyridin-3-yl)-1H-pyrrolo[2,3-b]pyridin-3-yl)imidazo[1,2-a]pyridine-3-carboxamide [1247];
6-(5-(4-methylpyridin-3-yl)-1H-pyrrolo[2,3-b]pyridin-3-yl)imidazo[1,2-a]pyrimidine [1248];
4-fluoro-1-isopropyl-2-methyl-6-(5-(4-methylpyridin-3-yl)-1H-pyrrolo[2,3-b]pyridin-3-yl)-1H-benzo[d]imidazole [1249];
4-fluoro-2-methyl-1-(1-methylpiperidin-4-yl)-6-(5-(4-methylpyridin-3-yl)-1H-pyrrolo[2,3-b]pyridin-3-yl)-1H-benzo[d]imidazole [1250];
1-isopropyl-2-methyl-6-(5-(4-methylpyridin-3-yl)-1H-pyrrolo[2,3-b]pyridin-3-yl)-1H-imidazo[4,5-b]pyridine [1251];
1-isopropyl-2-methyl-6-(5-(4-methylpyridin-3-yl)-1H-pyrrolo[2,3-b]pyridin-3-yl)-1H-imidazo[4,5-c]pyridine [1252];
6-(5-(4-methylpyridin-3-yl)-1H-pyrrolo[2,3-b]pyridin-3-yl)-[1,2,4]triazolo[1,5-a]pyridine [1253];
7-(5-(4-methylpyridin-3-yl)-1H-pyrrolo[2,3-b]pyridin-3-yl)-[1,2,4]triazolo[1,5-a]pyridine [1254];
1-methyl-6-(5-(4-methylpyridin-3-yl)-1H-pyrrolo[2,3-b]pyridin-3-yl)-1H-benzo[d][1,2,3]triazole [1255];
6-(5-(4-methylpyridin-3-yl)-1H-pyrrolo[2,3-b]pyridin-3-yl)imidazo[1,2-b]pyridazine [1256];
5-(4-methylpyridin-3-yl)-3-(pyrazolo[1,5-a]pyridin-5-yl)-1H-pyrrolo[2,3-b]pyridine [1257];
3-(3-(1-methyl-1H-pyrazol-4-yl)pyrazolo[1,5-a]pyridin-5-yl)-5-(4-methylpyridin-3-yl)-1H-pyrrolo[2,3-b]pyridine [1258];
5-(4-methylpyridin-3-yl)-3-(3-(pyridin-3-yl)pyrazolo[1,5-a]pyridin-5-yl)-1H-pyrrolo[2,3-b]pyridine [1259];
N-(2,2-difluoroethyl)-5-(5-(4-methylpyridin-3-yl)-1H-pyrrolo[2,3-b]pyridin-3-yl)pyrazolo[1,5-a]pyridine-3-carboxamide [1260];
(R)-5-(5-(4-methylpyridin-3-yl)-1H-pyrrolo[2,3-b]pyridin-3-yl)-N-(1,1,1-trifluoropropan-2-yl)pyrazolo[1,5-a]pyridine-3-carboxamide [1261];
N-(1-methylpiperidin-4-yl)-5-(5-(4-methylpyridin-3-yl)-1H-pyrrolo[2,3-b]pyridin-3-yl)pyrazolo[1,5-a]pyridine-3-carboxamide [1262];
5-(5-(4-methylpyridin-3-yl)-1H-pyrrolo[2,3-b]pyridin-3-yl)-N-(pyridin-3-yl)pyrazolo[1,5-a]pyridine-3-carboxamide [1263];
(5-(5-(4-methylpyridin-3-yl)-1H-pyrrolo[2,3-b]pyridin-3-yl)pyrazolo[1,5-a]pyridin-3-yl)(piperidin-1-yl)methanone [1264];
(4-methylpiperazin-1-yl)(5-(5-(4-methylpyridin-3-yl)-1H-pyrrolo[2,3-b]pyridin-3-yl)pyrazolo[1,5-a]pyridin-3-yl)methanone [1265];
(5-(5-(4-methylpyridin-3-yl)-1H-pyrrolo[2,3-b]pyridin-3-yl)pyrazolo[1,5-a]pyridin-3-yl)(morpholino)methanone [1266];
6-(5-(4-methylpyridin-3-yl)-1H-pyrrolo[2,3-b]pyridin-3-yl)spiro[indene-1,4′-piperidin]-3(2H)-one [1268];
1′-methyl-6-(5-(4-methylpyridin-3-yl)-1H-pyrrolo[2,3-b]pyridin-3-yl)spiro[indene-1,4′-piperidin]-3(2H)-one [1269];
5-(5-(4-methylpyridin-3-yl)-1H-pyrrolo[2,3-b]pyridin-3-yl)isoindolin-1-one [1270];
3,3-dimethyl-5-(5-(4-methylpyridin-3-yl)-1H-pyrrolo[2,3-b]pyridin-3-yl)isoindolin-1-one [1271];
6′-(5-(4-methylpyridin-3-yl)-1H-pyrrolo[2,3-b]pyridin-3-yl)spiro[cyclohexane-1,1′-isoindolin]-3′-one [1272];
3-(chroman-6-yl)-5-(4-methylpyridin-3-yl)-1H-pyrrolo[2,3-b]pyridine [1273];
6-(5-(4-methylpyridin-3-yl)-1H-pyrrolo[2,3-b]pyridin-3-yl)-3,4-dihydroisoquinolin-1(2H)-one [1274];
4,4-dimethyl-6-(5-(4-methylpyridin-3-yl)-1H-pyrrolo[2,3-b]pyridin-3-yl)-3,4-dihydroisoquinolin-1(2H)-one [1275];
7-(5-(4-methylpyridin-3-yl)-1H-pyrrolo[2,3-b]pyridin-3-yl)spiro[chromane-2,4′-piperidin]-4-one [1276];
8-(5-(4-methylpyridin-3-yl)-1H-pyrrolo[2,3-b]pyridin-3-yl)-3,4-dihydrobenzo[f][1,4]oxazepin-5(2H)-one [1277];
6-(5-(4-methylpyridin-3-yl)-1H-pyrrolo[2,3-b]pyridin-3-yl)quinoline [1278];
6-(5-(4-methylpyridin-3-yl)-1H-pyrrolo[2,3-b]pyridin-3-yl)quinoxaline [1279];
6-(5-(4-methylpyridin-3-yl)-1H-pyrrolo[2,3-b]pyridin-3-yl)quinazoline [1280];
4-methoxy-6-(5-(4-methylpyridin-3-yl)-1H-pyrrolo[2,3-b]pyridin-3-yl)quinazoline [1281];
4-(4-methylpiperazin-1-yl)-6-(5-(4-methylpyridin-3-yl)-1H-pyrrolo[2,3-b]pyridin-3-yl)quinazoline [1282];
4-((1-methylpiperidin-4-yl)oxy)-6-(5-(4-methylpyridin-3-yl)-1H-pyrrolo[2,3-b]pyridin-3-yl)quinazoline [1283];
N-(1-methylpiperidin-4-yl)-6-(5-(4-methylpyridin-3-yl)-1H-pyrrolo[2,3-b]pyridin-3-yl)quinazolin-4-amine [1284];
5-(5-(2,4-dimethylpyridin-3-yl)-1H-pyrrolo[2,3-b]pyridin-3-yl)-2-methyl-2H-indazole [1285];
2-(5-(2,4-dimethylpyridin-3-yl)-1H-pyrrolo[2,3-b]pyridin-3-yl)thieno[2,3-c]pyridine [1286];
7-(5-(2,4-dimethylpyridin-3-yl)-1H-pyrrolo[2,3-b]pyridin-3-yl)-3,4-dihydropyrrolo[1,2-a]pyrazin-1(2H)-one [1287];
6-(5-(2,4-dimethylpyridin-3-yl)-1H-pyrrolo[2,3-b]pyridin-3-yl)imidazo[1,2-a]pyridine [1288];
N-(2,2-difluoroethyl)-6-(5-(2,4-dimethylpyridin-3-yl)-1H-pyrrolo[2,3-b]pyridin-3-yl)imidazo[1,2-a]pyridine-3-carboxamide [1289];
6-(5-(2,4-dimethylpyridin-3-yl)-1H-pyrrolo[2,3-b]pyridin-3-yl)imidazo[1,2-a]pyrimidine [1290];
6-(5-(2,4-dimethylpyridin-3-yl)-1H-pyrrolo[2,3-b]pyridin-3-yl)-4-fluoro-1-isopropyl-2-methyl-1H-benzo[d]imidazole [1291];
6-(5-(2,4-dimethylpyridin-3-yl)-1H-pyrrolo[2,3-b]pyridin-3-yl)-4-fluoro-2-methyl-1-(1-methylpiperidin-4-yl)-1H-benzo[d]imidazole [1292];
6-(5-(2,4-dimethylpyridin-3-yl)-1H-pyrrolo[2,3-b]pyridin-3-yl)-1-isopropyl-2-methyl-1H-imidazo[4,5-b]pyridine [1293];
6-(5-(2,4-dimethylpyridin-3-yl)-1H-pyrrolo[2,3-b]pyridin-3-yl)-1-isopropyl-2-methyl-1H-imidazo[4,5-c]pyridine [1294];
6-(5-(2,4-dimethylpyridin-3-yl)-1H-pyrrolo[2,3-b]pyridin-3-yl)-[1,2,4]triazolo[1,5-a]pyridine [1295];
7-(5-(2,4-dimethylpyridin-3-yl)-1H-pyrrolo[2,3-b]pyridin-3-yl)-[1,2,4]triazolo[1,5-a]pyridine [1296];
6-(5-(2,4-dimethylpyridin-3-yl)-1H-pyrrolo[2,3-b]pyridin-3-yl)-1-methyl-1H-benzo[d][1,2,3]triazole [1297];
6-(5-(2,4-dimethylpyridin-3-yl)-1H-pyrrolo[2,3-b]pyridin-3-yl)imidazo[1,2-b]pyridazine [1298];
5-(2,4-dimethylpyridin-3-yl)-3-(pyrazolo[1,5-a]pyridin-5-yl)-1H-pyrrolo[2,3-b]pyridine [1299];
5-(2,4-dimethylpyridin-3-yl)-3-(3-(1-methyl-1H-pyrazol-4-yl)pyrazolo[1,5-a]pyridin-5-yl)-1H-pyrrolo[2,3-b]pyridine [1300];
5-(2,4-dimethylpyridin-3-yl)-3-(3-(pyridin-3-yl)pyrazolo[1,5-a]pyridin-5-yl)-1H-pyrrolo[2,3-b]pyridine [1301];
N-(2,2-difluoroethyl)-5-(5-(2,4-dimethylpyridin-3-yl)-1H-pyrrolo[2,3-b]pyridin-3-yl)pyrazolo[1,5-a]pyridine-3-carboxamide [1302];
(R)-5-(5-(2,4-dimethylpyridin-3-yl)-1H-pyrrolo[2,3-b]pyridin-3-yl)-N-(1,1,1-trifluoropropan-2-yl)pyrazolo[1,5-a]pyridine-3-carboxamide [1303];
5-(5-(2,4-dimethylpyridin-3-yl)-1H-pyrrolo[2,3-b]pyridin-3-yl)-N-(1-methylpiperidin-4-yl)pyrazolo[1,5-a]pyridine-3-carboxamide [1304];
5-(5-(2,4-dimethylpyridin-3-yl)-1H-pyrrolo[2,3-b]pyridin-3-yl)-N-(pyridin-3-yl)pyrazolo[1,5-a]pyridine-3-carboxamide [1305];
(5-(5-(2,4-dimethylpyridin-3-yl)-1H-pyrrolo[2,3-b]pyridin-3-yl)pyrazolo[1,5-a]pyridin-3-yl)(piperidin-1-yl)methanone [1306];
(5-(5-(2,4-dimethylpyridin-3-yl)-1H-pyrrolo[2,3-b]pyridin-3-yl)pyrazolo[1,5-a]pyridin-3-yl)(4-methylpiperazin-1-yl)methanone [1307];
(5-(5-(2,4-dimethylpyridin-3-yl)-1H-pyrrolo[2,3-b]pyridin-3-yl)pyrazolo[1,5-a]pyridin-3-yl)(morpholino)methanone [1308];
6-(5-(2,4-dimethylpyridin-3-yl)-1H-pyrrolo[2,3-b]pyridin-3-yl)spiro[indene-1,4′-piperidin]-3(2H)-one [1310];
6-(5-(2,4-dimethylpyridin-3-yl)-1H-pyrrolo[2,3-b]pyridin-3-yl)-1′-methylspiro[indene-1,4′-piperidin]-3(2H)-one [1311];
5-(5-(2,4-dimethylpyridin-3-yl)-1H-pyrrolo[2,3-b]pyridin-3-yl)isoindolin-1-one [1312];
5-(5-(2,4-dimethylpyridin-3-yl)-1H-pyrrolo[2,3-b]pyridin-3-yl)-3,3-dimethylisoindolin-1-one [1313];
6′-(5-(2,4-dimethylpyridin-3-yl)-1H-pyrrolo[2,3-b]pyridin-3-yl)spiro[cyclohexane-1,1′-isoindolin]-3′-one [1314];
3-(chroman-6-yl)-5-(2,4-dimethylpyridin-3-yl)-1H-pyrrolo[2,3-b]pyridine [1315];
6-(5-(2,4-dimethylpyridin-3-yl)-1H-pyrrolo[2,3-b]pyridin-3-yl)-3,4-dihydroisoquinolin-1(2H)-one [1316];
6-(5-(2,4-dimethylpyridin-3-yl)-1H-pyrrolo[2,3-b]pyridin-3-yl)-4,4-dimethyl-3,4-dihydroisoquinolin-1(2H)-one [1317];
7-(5-(2,4-dimethylpyridin-3-yl)-1H-pyrrolo[2,3-b]pyridin-3-yl)spiro[chromane-2,4′-piperidin]-4-one [1318];
8-(5-(2,4-dimethylpyridin-3-yl)-1H-pyrrolo[2,3-b]pyridin-3-yl)-3,4-dihydrobenzo[1,4]oxazepin-5(2H)-one [1319];
6-(5-(2,4-dimethylpyridin-3-yl)-1H-pyrrolo[2,3-b]pyridin-3-yl)quinoline [1320];
6-(5-(2,4-dimethylpyridin-3-yl)-1H-pyrrolo[2,3-b]pyridin-3-yl)quinoxaline [1321];
6-(5-(2,4-dimethylpyridin-3-yl)-1H-pyrrolo[2,3-b]pyridin-3-yl)quinazoline [1322];
6-(5-(2,4-dimethylpyridin-3-yl)-1H-pyrrolo[2,3-b]pyridin-3-yl)-4-methoxyquinazoline [1323];
6-(5-(2,4-dimethylpyridin-3-yl)-1H-pyrrolo[2,3-b]pyridin-3-yl)-4-(4-methylpiperazin-1-yl)quinazoline [1324];
6-(5-(2,4-dimethylpyridin-3-yl)-1H-pyrrolo[2,3-b]pyridin-3-yl)-4-((1-methylpiperidin-4-yl)oxy)quinazoline [1325];
6-(5-(2,4-dimethylpyridin-3-yl)-1H-pyrrolo[2,3-b]pyridin-3-yl)-N-(1-methylpiperidin-4-yl)quinazolin-4-amine [1326];
2-methyl-5-(5-(6-(4-methylpiperazin-1-yl)pyridin-3-yl)-1H-pyrrolo[2,3-b]pyridin-3-yl)-2H-indazole [1327];
2-(5-(6-(4-methylpiperazin-1-yl)pyridin-3-yl)-1H-pyrrolo[2,3-b]pyridin-3-yl)thieno[2,3-c]pyridine [1328];
7-(5-(6-(4-methylpiperazin-1-yl)pyridin-3-yl)-1H-pyrrolo[2,3-b]pyridin-3-yl)-3,4-dihydropyrrolo[1,2-a]pyrazin-1(2H)-one [1329];
6-(5-(6-(4-methylpiperazin-1-yl)pyridin-3-yl)-1H-pyrrolo[2,3-b]pyridin-3-yl)imidazo[1,2-a]pyridine [1330];
N-(2,2-difluoroethyl)-6-(5-(6-(4-methylpiperazin-1-yl)pyridin-3-yl)-1H-pyrrolo[2,3-b]pyridin-3-yl)imidazo[1,2-a]pyridine-3-carboxamide [1331];
6-(5-(6-(4-methylpiperazin-1-yl)pyridin-3-yl)-1H-pyrrolo[2,3-b]pyridin-3-yl)imidazo[1,2-a]pyrimidine [1332];
4-fluoro-1-isopropyl-2-methyl-6-(5-(6-(4-methylpiperazin-1-yl)pyridin-3-yl)-1H-pyrrolo[2,3-b]pyridin-3-yl)-1H-benzo[d]imidazole [1333];
4-fluoro-2-methyl-6-(5-(6-(4-methylpiperazin-1-yl)pyridin-3-yl)-1H-pyrrolo[2,3-b]pyridin-3-yl)-1-(1-methylpiperidin-4-yl)-1H-benzo[d]imidazole [1334];
1-isopropyl-2-methyl-6-(5-(6-(4-methylpiperazin-1-yl)pyridin-3-yl)-1H-pyrrolo[2,3-b]pyridin-3-yl)-1H-imidazo[4,5-b]pyridine [1335];
1-isopropyl-2-methyl-6-(5-(6-(4-methylpiperazin-1-yl)pyridin-3-yl)-1H-pyrrolo[2,3-b]pyridin-3-yl)-1H-imidazo[4,5-c]pyridine [1336];
6-(5-(6-(4-methylpiperazin-1-yl)pyridin-3-yl)-1H-pyrrolo[2,3-b]pyridin-3-yl)-[1,2,4]triazolo[1,5-a]pyridine [1337];
7-(5-(6-(4-methylpiperazin-1-yl)pyridin-3-yl)-1H-pyrrolo[2,3-b]pyridin-3-yl)-[1,2,4]triazolo[1,5-a]pyridine [1338];
1-methyl-6-(5-(6-(4-methylpiperazin-1-yl)pyridin-3-yl)-1H-pyrrolo[2,3-b]pyridin-3-yl)-1H-benzo[d][1,2,3]triazole [1339];
6-(5-(6-(4-methylpiperazin-1-yl)pyridin-3-yl)-1H-pyrrolo[2,3-b]pyridin-3-yl)imidazo[1,2-b]pyridazine [1340];
5-(6-(4-methylpiperazin-1-yl)pyridin-3-yl)-3-(pyrazolo[1,5-a]pyridin-5-yl)-1H-pyrrolo[2,3-b]pyridine [1341];
3-(3-(1-methyl-1H-pyrazol-4-yl)pyrazolo[1,5-a]pyridin-5-yl)-5-(6-(4-methylpiperazin-1-yl)pyridin-3-yl)-1H-pyrrolo[2,3-b]pyridine [1342];
5-(6-(4-methylpiperazin-1-yl)pyridin-3-yl)-3-(3-(pyridin-3-yl)pyrazolo[1,5-a]pyridin-5-yl)-1H-pyrrolo[2,3-1)]pyridine [1343];
N-(2,2-difluoroethyl)-5-(5-(6-(4-methylpiperazin-1-yl)pyridin-3-yl)-1H-pyrrolo[2,3-b]pyridin-3-yl)pyrazolo[1,5-a]pyridine-3-carboxamide [1344];
(R)-5-(5-(6-(4-methylpiperazin-1-yl)pyridin-3-yl)-1H-pyrrolo[2,3-b]pyridin-3-yl)-N-(1,1,1-trifluoropropan-2-yl)pyrazolo[1,5-a]pyridine-3-carboxamide [1345];
5-(5-(6-(4-methylpiperazin-1-yl)pyridin-3-yl)-1H-pyrrolo[2,3-b]pyridin-3-yl)-N-(1-methylpiperidin-4-yl)pyrazolo[1,5-a]pyridine-3-carboxamide [1346];
5-(5-(6-(4-methylpiperazin-1-yl)pyridin-3-yl)-1H-pyrrolo[2,3-b]pyridin-3-yl)-N-(pyridin-3-yl)pyrazolo[1,5-a]pyridine-3-carboxamide [1347];
(5-(5-(6-(4-methylpiperazin-1-yl)pyridin-3-yl)-1H-pyrrolo[2,3-b]pyridin-3-yl)pyrazolo[1,5-a]pyridin-3-yl)(piperidin-1-yl)methanone [1348];
(4-methylpiperazin-1-yl)(5-(5-(6-(4-methylpiperazin-1-yl)pyridin-3-yl)-1H-pyrrolo[2,3-b]pyridin-3-yl)pyrazolo[1,5-a]pyridin-3-yl)methanone [1349];
(5-(5-(6-(4-methylpiperazin-1-yl)pyridin-3-yl)-1H-pyrrolo[2,3-b]pyridin-3-yl)pyrazolo[1,5-a]pyridin-3-yl)(morpholino)methanone [1350];
6-(5-(6-(4-methylpiperazin-1-yl)pyridin-3-yl)-1H-pyrrolo[2,3-b]pyridin-3-yl)spiro[indene-1,4′-piperidin]-3(2H)-one [1352];
1′-methyl-6-(5-(6-(4-methylpiperazin-1-yl)pyridin-3-yl)-1H-pyrrolo[2,3-b]pyridin-3-yl)spiro[indene-1,4′-piperidin]-3(2H)-one [1353];
5-(5-(6-(4-methylpiperazin-1-yl)pyridin-3-yl)-1H-pyrrolo[2,3-b]pyridin-3-yl)isoindolin-1-one [1354];
3,3-dimethyl-5-(5-(6-(4-methylpiperazin-1-yl)pyridin-3-yl)-1H-pyrrolo[2,3-b]pyridin-3-yl)isoindolin-1-one [1355];
6′-(5-(6-(4-methylpiperazin-1-yl)pyridin-3-yl)-1H-pyrrolo[2,3-b]pyridin-3-yl)spiro[cyclohexane-1,1′-isoindolin]-3′-one [1356];
3-(chroman-6-yl)-5-(6-(4-methylpiperazin-1-yl)pyridin-3-yl)-1H-pyrrolo[2,3-b]pyridine [1357];
6-(5-(6-(4-methylpiperazin-1-yl)pyridin-3-yl)-1H-pyrrolo[2,3-b]pyridin-3-yl)-3,4-dihydroisoquinolin-1(2H)-one [1358];
4,4-dimethyl-6-(5-(6-(4-methylpiperazin-1-yl)pyridin-3-yl)-1H-pyrrolo[2,3-b]pyridin-3-yl)-3,4-dihydroisoquinolin-1(2H)-one [1359];
7-(5-(6-(4-methylpiperazin-1-yl)pyridin-3-yl)-1H-pyrrolo[2,3-b]pyridin-3-yl)spiro[chromane-2,4′-piperidin]-4-one [1360];
8-(5-(6-(4-methylpiperazin-1-yl)pyridin-3-yl)-1H-pyrrolo[2,3-b]pyridin-3-yl)-3,4-dihydrobenzo[1,4]oxazepin-5(2H)-one [1361];
6-(5-(6-(4-methylpiperazin-1-yl)pyridin-3-yl)-1H-pyrrolo[2,3-b]pyridin-3-yl)quinoline [1362];
6-(5-(6-(4-methylpiperazin-1-yl)pyridin-3-yl)-1H-pyrrolo[2,3-b]pyridin-3-yl)quinoxaline [1363];
6-(5-(6-(4-methylpiperazin-1-yl)pyridin-3-yl)-1H-pyrrolo[2,3-b]pyridin-3-yl)quinazoline [1364];
4-methoxy-6-(5-(6-(4-methylpiperazin-1-yl)pyridin-3-yl)-1H-pyrrolo[2,3-b]pyridin-3-yl)quinazoline [1365];
4-(4-methylpiperazin-1-yl)-6-(5-(6-(4-methylpiperazin-1-yl)pyridin-3-yl)-1H-pyrrolo[2,3-b]pyridin-3-yl)quinazoline [1366];
6-(5-(6-(4-methylpiperazin-1-yl)pyridin-3-yl)-1H-pyrrolo[2,3-b]pyridin-3-yl)-4-((1-methylpiperidin-4-yl)oxy)quinazoline [1367];
6-(5-(6-(4-methylpiperazin-1-yl)pyridin-3-yl)-1H-pyrrolo[2,3-b]pyridin-3-yl)-N-(1-methylpiperidin-4-yl)quinazolin-4-amine [1368];
2-methyl-5-(5-(2-(4-methylpiperazin-1-yl)pyridin-4-yl)-1H-pyrrolo[2,3-b]pyridin-3-yl)-2H-indazole [1369];
2-(5-(2-(4-methylpiperazin-1-yl)pyridin-4-yl)-1H-pyrrolo[2,3-b]pyridin-3-yl)thieno[2,3-c]pyridine [1370];
7-(5-(2-(4-methylpiperazin-1-yl)pyridin-4-yl)-1H-pyrrolo[2,3-b]pyridin-3-yl)-3,4-dihydropyrrolo[1,2-a]pyrazin-1(2H)-one [1371];
6-(5-(2-(4-methylpiperazin-1-yl)pyridin-4-yl)-1H-pyrrolo[2,3-b]pyridin-3-yl)imidazo[1,2-a]pyridine [1372];
N-(2,2-difluoroethyl)-6-(5-(2-(4-methylpiperazin-1-yl)pyridin-4-yl)-1H-pyrrolo[2,3-b]pyridin-3-yl)imidazo[1,2-a]pyridine-3-carboxamide [1373];
6-(5-(2-(4-methylpiperazin-1-yl)pyridin-4-yl)-1H-pyrrolo[2,3-b]pyridin-3-yl)imidazo[1,2-a]pyrimidine [1374];
4-fluoro-1-isopropyl-2-methyl-6-(5-(2-(4-methylpiperazin-1-yl)pyridin-4-yl)-1H-pyrrolo[2,3-b]pyridin-3-yl)-1H-benzo[d]imidazole [1375];
4-fluoro-2-methyl-6-(5-(2-(4-methylpiperazin-1-yl)pyridin-4-yl)-1H-pyrrolo[2,3-b]pyridin-3-yl)-1-(1-methylpiperidin-4-yl)-1H-benzo[d]imidazole [1376];
1-isopropyl-2-methyl-6-(5-(2-(4-methylpiperazin-1-yl)pyridin-4-yl)-1H-pyrrolo[2,3-b]pyridin-3-yl)-1H-imidazo[4,5-b]pyridine [1377];
1-isopropyl-2-methyl-6-(5-(2-(4-methylpiperazin-1-yl)pyridin-4-yl)-1H-pyrrolo[2,3-b]pyridin-3-yl)-1H-imidazo[4,5-c]pyridine [1378];
6-(5-(2-(4-methylpiperazin-1-yl)pyridin-4-yl)-1H-pyrrolo[2,3-b]pyridin-3-yl)-[1,2,4]triazolo[1,5-a]pyridine [1379];
7-(5-(2-(4-methylpiperazin-1-yl)pyridin-4-yl)-1H-pyrrolo[2,3-b]pyridin-3-yl)-[1,2,4]triazolo[1,5-a]pyridine [1380];
1-methyl-6-(5-(2-(4-methylpiperazin-1-yl)pyridin-4-yl)-1H-pyrrolo[2,3-b]pyridin-3-yl)-1H-benzo[d][1,2,3]triazole [1381];
6-(5-(2-(4-methylpiperazin-1-yl)pyridin-4-yl)-1H-pyrrolo[2,3-b]pyridin-3-yl)imidazo[1,2-b]pyridazine [1382];
5-(2-(4-methylpiperazin-1-yl)pyridin-4-yl)-3-(pyrazolo[1,5-a]pyridin-5-yl)-1H-pyrrolo[2,3-b]pyridine [1383];
3-(3-(1-methyl-1H-pyrazol-4-yl)pyrazolo[1,5-a]pyridin-5-yl)-5-(2-(4-methylpiperazin-1-yl)pyridin-4-yl)-1H-pyrrolo[2,3-b]pyridine [1384];
5-(2-(4-methylpiperazin-1-yl)pyridin-4-yl)-3-(3-(pyridin-3-yl)pyrazolo[1,5-a]pyridin-5-yl)-1H-pyrrolo[2,3-b]pyridine [1385];
N-(2,2-difluoroethyl)-5-(5-(2-(4-methylpiperazin-1-yl)pyridin-4-yl)-1H-pyrrolo[2,3-b]pyridin-3-yl)pyrazolo[1,5-a]pyridine-3-carboxamide [1386];
(R)-5-(5-(2-(4-methylpiperazin-1-yl)pyridin-4-yl)-1H-pyrrolo[2,3-b]pyridin-3-yl)-N-(1,1,1-trifluoropropan-2-yl)pyrazolo[1,5-a]pyridine-3-carboxamide [1387];
5-(5-(2-(4-methylpiperazin-1-yl)pyridin-4-yl)-1H-pyrrolo[2,3-b]pyridin-3-yl)-N-(1-methylpiperidin-4-yl)pyrazolo[1,5-a]pyridine-3-carboxamide [1388];
5-(5-(2-(4-methylpiperazin-1-yl)pyridin-4-yl)-1H-pyrrolo[2,3-b]pyridin-3-yl)-N-(pyridin-3-yl)pyrazolo[1,5-a]pyridine-3-carboxamide [1389];
(5-(5-(2-(4-methylpiperazin-1-yl)pyridin-4-yl)-1H-pyrrolo[2,3-b]pyridin-3-yl)pyrazolo[1,5-a]pyridin-3-yl)(piperidin-1-yl)methanone [1390];
(4-methylpiperazin-1-yl)(5-(5-(2-(4-methylpiperazin-1-yl)pyridin-4-yl)-1H-pyrrolo[2,3-b]pyridin-3-yl)pyrazolo[1,5-a]pyridin-3-yl)methanone [1391];
(5-(5-(2-(4-methylpiperazin-1-yl)pyridin-4-yl)-1H-pyrrolo[2,3-b]pyridin-3-yl)pyrazolo[1,5-a]pyridin-3-yl)(morpholino)methanone [1392];
6-(5-(2-(4-methylpiperazin-1-yl)pyridin-4-yl)-1H-pyrrolo[2,3-b]pyridin-3-yl)spiro[indene-1,4′-piperidin]-3(2H)-one [1394];
1′-methyl-6-(5-(2-(4-methylpiperazin-1-yl)pyridin-4-yl)-1H-pyrrolo[2,3-b]pyridin-3-yl)spiro[indene-1,4′-piperidin]-3(2H)-one [1395];
5-(5-(2-(4-methylpiperazin-1-yl)pyridin-4-yl)-1H-pyrrolo[2,3-b]pyridin-3-yl)isoindolin-1-one [1396];
3,3-dimethyl-5-(5-(2-(4-methylpiperazin-1-yl)pyridin-4-yl)-1H-pyrrolo[2,3-b]pyridin-3-yl)isoindolin-1-one [1397];
6′-(5-(2-(4-methylpiperazin-1-yl)pyridin-4-yl)-1H-pyrrolo[2,3-b]pyridin-3-yl)spiro[cyclohexane-1,1′-isoindolin]-3′-one [1398];
3-(chroman-6-yl)-5-(2-(4-methylpiperazin-1-yl)pyridin-4-yl)-1H-pyrrolo[2,3-b]pyridine [1399];
6-(5-(2-(4-methylpiperazin-1-yl)pyridin-4-yl)-1H-pyrrolo[2,3-b]pyridin-3-yl)-3,4-dihydroisoquinolin-1(2H)-one [1400];
4,4-dimethyl-6-(5-(2-(4-methylpiperazin-1-yl)pyridin-4-yl)-1H-pyrrolo[2,3-b]pyridin-3-yl)-3,4-dihydroisoquinolin-1(2H)-one [1401];
7-(5-(2-(4-methylpiperazin-1-yl)pyridin-4-yl)-1H-pyrrolo[2,3-b]pyridin-3-yl)spiro[chromane-2,4′-piperidin]-4-one [1402];
8-(5-(2-(4-methylpiperazin-1-yl)pyridin-4-yl)-1H-pyrrolo[2,3-b]pyridin-3-yl)-3,4-dihydrobenzo[1,4]oxazepin-5(2H)-one [1403];
6-(5-(2-(4-methylpiperazin-1-yl)pyridin-4-yl)-1H-pyrrolo[2,3-b]pyridin-3-yl)quinoline [1404];
6-(5-(2-(4-methylpiperazin-1-yl)pyridin-4-yl)-1H-pyrrolo[2,3-b]pyridin-3-yl)quinoxaline [1405];
6-(5-(2-(4-methylpiperazin-1-yl)pyridin-4-yl)-1H-pyrrolo[2,3-b]pyridin-3-yl)quinazoline [1406];
4-methoxy-6-(5-(2-(4-methylpiperazin-1-yl)pyridin-4-yl)-1H-pyrrolo[2,3-b]pyridin-3-yl)quinazoline [1407];
4-(4-methylpiperazin-1-yl)-6-(5-(2-(4-methylpiperazin-1-yl)pyridin-4-yl)-1H-pyrrolo[2,3-b]pyridin-3-yl)quinazoline [1408];
6-(5-(2-(4-methylpiperazin-1-yl)pyridin-4-yl)-1H-pyrrolo[2,3-b]pyridin-3-yl)-4-((1-methylpiperidin-4-yl)oxy)quinazoline [1409];
6-(5-(2-(4-methylpiperazin-1-yl)pyridin-4-yl)-1H-pyrrolo[2,3-b]pyridin-3-yl)-N-(1-methylpiperidin-4-yl)quinazolin-4-amine [1410];
(8-methyl-3,8-diazabicyclo[3.2.1]octan-3-yl)(3-(thieno[2,3-c]pyridin-2-yl)-1H-pyrrolo[2,3-b]pyridin-5-yl)methanone [2041];
3-(4-fluoro-1-isopropyl-2-methyl-1H-benzo[d]imidazol-6-yl)-1H-pyrrolo[2,3-b]pyridine-5-carboxylic acid [2043];
(3-(4-fluoro-1-isopropyl-2-methyl-1H-benzo[d]imidazol-6-yl)-1H-pyrrolo[2,3-b]pyridin-5-yl)(8-methyl-3,8-diazabicyclo[3.2.1]octan-3-yl)methanone [2044];
3-(3-(4-methylpiperazine-1-carbonyl)pyrazolo[1,5-a]pyridin-5-yl)-1H-pyrrolo[2,3-b]pyridine-5-carbonitrile [2046];
(5-(5-methoxy-1H-pyrrolo[2,3-b]pyridin-3-yl)pyrazolo[1,5-a]pyridin-3-yl)(4-methylpiperazin-1-yl)methanone [2047];
(8-methyl-3,8-diazabicyclo[3.2.1]octan-3-yl)(3-(pyrazolo[1,5-a]pyridin-5-yl)-1H-pyrrolo[2,3-b]pyridin-5-yl)methanone [2049]; and
(4-methylpiperazin-1-yl)(5-(5-(trifluoromethyl)-1H-pyrrolo[2,3-b]pyridin-3-yl)pyrazolo[1,5-a]pyridin-3-yl)methanone [2050]; or a pharmaceutically acceptable salt thereof.

23. A pharmaceutical composition comprising a therapeutically effective amount of a compound of claim 1, or a pharmaceutically acceptable salt thereof, and a pharmaceutically acceptable excipient.

24. A pharmaceutical composition comprising a therapeutically effective amount of a compound of claim 7, or a pharmaceutically acceptable salt thereof, and a pharmaceutically acceptable excipient.

25. A pharmaceutical composition comprising a therapeutically effective amount of a compound of claim 10, or a pharmaceutically acceptable salt thereof, and a pharmaceutically acceptable excipient.

26. A pharmaceutical composition comprising a therapeutically effective amount of a compound of claim 15, or a pharmaceutically acceptable salt thereof, and a pharmaceutically acceptable excipient.

27. A method of treating a disorder or disease in a patient, wherein the disorder or disease is selected from the group consisting of: a neurological disorder, diabetes, and cancer, the method comprising administering to the patient a therapeutically effective amount of a compound of claim 1, or a pharmaceutically acceptable salt, or a pharmaceutical composition of claim 23.

28. A method of treating a disorder or disease in a patient, wherein the disorder or disease is selected from the group consisting of: a neurological disorder, diabetes, and cancer, the method comprising administering to the patient a therapeutically effective amount of a compound of claim 7, or a pharmaceutically acceptable salt, or a pharmaceutical composition of claim 24.

29. A method of treating a disorder or disease in a patient, wherein the disorder or disease is selected from the group consisting of: a neurological disorder, diabetes, and cancer, the method comprising administering to the patient a therapeutically effective amount of a compound of claim 10, or a pharmaceutically acceptable salt, or a pharmaceutical composition of claim 25.

30. A method of treating a disorder or disease in a patient, wherein the disorder or disease is selected from the group consisting of: a neurological disorder, diabetes, and cancer, the method comprising administering to the patient a therapeutically effective amount of a compound of claim 15, or a pharmaceutically acceptable salt, or a pharmaceutical composition of claim 26.
